(19) United States
(12) Patent Application Publication
(10) Pub. No.: US 2006/0073582 A1
(43) Pub. Date: Apr. 6, 2006

(54) Rab9 protein crystal structures and methods for identifying Rab9 modulators

(75) Inventors: Edward J. Meehan, Huntsville, AL (US); Liqing Chen, Huntsville, AL (US); Enrico L. DiGiammarino, Woodstock, GA (US); Thomas W. Hodge, Athens, GA (US)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: The University of Alamaba in Huntsville, Huntsville, AL (US)

(21) Appl. No.: 11/154,203

(22) Filed: Jun. 16, 2005

Related U.S. Application Data
(60) Provisional application No. 60/603,904, filed on Aug. 24, 2004.
(60) Provisional application No. 60/581,961, filed on Jun. 22, 2004.

(51) Int. Cl. G01N 33/48; C12N 9/12; G06F 19/00

(52) U.S. Cl. 435/194; 702/19

(57) ABSTRACT

The present invention relates to crystalline Rab9 in complex with either GDP or the GTP analog Guanosine 5′-(β,γ-imido) triphosphate (GppNHp), the three dimensional coordinates and structures of Rab9 in Rab9-GDP and Rab9-GppNHp complexes, and uses thereof for drug design. In particular, the present invention relates to methods for producing Rab9 crystals of sufficient quality to obtain a determination of the three dimensional structure of Rab9 to a high resolution in both its GTP-bound (on/active) and GDP-bound (off/inactive) conformations. The present invention also relates to a computer-readable medium encoded with the three dimensional coordinates of Rab9 in Rab9-GDP and Rab9-GppNHp complexes wherein, using a graphical display software program, the three dimensional coordinates create an electronic file that can be visualized on a computer capable of representing the electronic file as a three dimensional image.

### CROSS-REFERENCE TO RELATED APPLICATIONS

This application claims the benefit of U.S. Provisional Application No. 60/603,904, filed Aug. 24, 2004; and U.S. Provisional Application No. 60/581,961, filed Jun. 22, 2004; each of which are hereby incorporated by reference.

### FEDERALLY SPONSORED RESEARCH OR DEVELOPMENT

The research underlying this invention was supported in part with funds from the National Science Foundation (NSF; NSF EPSCoR Program Grant No. EPS-0091853), the National Aeronautics and Space Administration (NASA; NASA Cooperative Agreement Number NCC8-243), and the United States Department of Energy (DOE; Contract No. W-31-109-Eng-38). The United States Government may have an interest in the subject matter of this invention.

### FIELD OF THE INVENTION

The invention relates to a high resolution crystal structure for human Rab9, and in particular to methods of use for this crystal structure for drug discovery.

### BACKGROUND OF THE INVENTION

Rab proteins are the largest subfamily of the Ras-like small GTPase superfamily, and serve as key regulators in vesicular transport (Lombardi et al. (1993) EMBO J. 12: 677-82). Rab proteins are thought to function in vesicle fusion and/or targeting events, a hypothesis bolstered by evidence that most organelles of the endocytic and secretory pathways bear distinct Rab proteins on their surfaces (Novick et al. (1980) Cell 21: 205-15; Plutner et al. (1991) J. Cell Biol. 115: 31-43; Rexach and Schekman (1991) J. Cell Biol. 114: 219-29; Segev (1991) Science 252: 1553-56; Gorvel et al. (1991) Cell 64: 915-25; Lombardi et al. (1993) EMBO J. 12: 677-82). Biochemical and genetic studies of chimeric and mutant Rab proteins have identified several hypervariable regions, including the N- and C-termini and the α3/β5 loop, that play an important role in determining functional specificity (Brennwald and Novick (1993) Nature 362: 560-63; Stenmark et al. EMBO J. 13: 575-83).

Rab proteins serve as molecular switches mediating tethering, docking, fusion, and motility of intracellular membranes by cycling between GTP-bound (on/active) and GDP-bound (off/inactive) conformations. (Pfeffer (2001) Trends Cell Biol. 11: 487-91; Pfeffer (1994) Curr. Opin. Cell Biol. 6: 522-26; Bourne et al. (1991) Nature 349: 117-27; Sprang (1997) Ann. Rev. Biochem. 66: 639-78). The active form is stabilized by additional hydrogen bond interactions with the γ-phosphate of GTP, mediated by serine residues in the phosphate-binding loop (P-loop) and switch I region, as well as an extensive hydrophobic interface between the switch I and II regions (Dumas et al. (1999) Structure Fold Des. 7: 413-23; Esters et al. (2000) J. Mol. Biol. 298: 111-21). The inactive conformation usually has displaced and mobile switch regions. (Stroupe and Brunger (2000) J. Mol. Biol. 304: 585-98).

One particular Rab protein, Rab9, was first identified in a screen of cDNA clones (Chavrier et al. (1990) Cell 62: 317-29). Rab9 is localized predominantly on the surface of late endosomes and stimulates the recycling of mannose-6-phosphate receptors (MPRs) from late endosomes to the trans-Golgi network (TGN) (Lombardi et al. (1993) EMBO J. 12: 677-82; Riederer et al. (1994) J. Cell Biol. 125: 573-82). Rab9 also interacts with the vesicle cargo selection protein TIP47, which has been shown to bind the cytoplasmic tail of the HIV envelope glycoprotein subunit gp41 (Blot et al. (2003) J. Virol. 77: 6931-45).

Recent interest on Rab9 has focused on its role in HIV-1, Ebola, Marburg and Measles virus replication, making drugs that target Rab9 of significant technical and commercial interest. However, no crystal structure of human Rab9 has been available that could be used for structure guided drug design. There is therefore a need for a method to determine a high resolution crystal structure of human Rab9, as well as methods of using of this crystal structure for drug discovery.

### SUMMARY OF THE INVENTION

The present invention relates to crystalline Rab9 in complex with either GDP or the GTP analog Guanosine 5′-(β,γ-imido) triphosphate (GppNHp), the three dimensional coordinates and structures of Rab9 in Rab9-GDP and Rab9-GppNHp complexes, and uses thereof for drug design. In particular, the present invention relates to methods for producing Rab9 crystals of sufficient quality to obtain a determination of the three dimensional structure of Rab9 to a high resolution in both its GTP-bound (on/active) and GDP-bound (off/inactive) conformations. The present invention also relates to a computer-readable medium encoded with the three dimensional coordinates of Rab9 in Rab9-GDP and Rab9-GppNHp complexes wherein, using a graphical display software program, the three dimensional coordinates create an electronic file that can be visualized on a computer capable of representing the electronic file as a three dimensional image.

In one embodiment, a crystal of a C-terminally truncated human Rab9 having the three dimensional atomic coordinates of Table 4 or Table 6, wherein said Rab9 comprises amino acids 1-177 of SEQ ID NO:1.

In another embodiment, a crystal of a Rab9-GDP complex is provided wherein the crystal effectively diffracts X-rays for the determination of the atomic coordinates of the Rab9-GDP complex to a resolution of less than about 1.75 Å, preferably between about 1.25 Å and about 1.75 Å, and most preferably about 1.25 Å.

In another embodiment, a crystal of a Rab9-GDP complex is provided having a space group of P₁ and a unit cell of dimensions a=38.40 Å, b=45.62 Å, c=51.22 Å, α=99.8°, β=107.20, and γ=101.8°.

In another embodiment, a Rab9-GDP complex is provided wherein the Rab9 has secondary structural elements that include six β-sheets and five α-helices, wherein the β-sheets are designated B1-B6 and wherein the α-helices are designated H1-H5, and wherein the β-sheets and the α-helices connect with a B1-H1-B2-B3-H2-B4-H3-B5-H4-B6-H5 topology.

In another embodiment, a crystal of a Rab9-GppNHp complex is provided wherein the crystal effectively diffracts X-rays for the determination of the atomic coordinates of the Rab9-GppNHp complex to a resolution of less than about 20 Å, preferably less than about 2 Å, more preferably between about 1.25 Å and about 2 Å, and most preferably about 1.73 Å.

In another embodiment, a crystal of a Rab9-GppNHp complex is provided having a space group of P2₁2₁2, and a unit cell of dimensions a=56.24 Å, b=76.60 Å, and c=174.35 Å.

In another embodiment, Rab9-GppNHp complex is provided wherein the Rab9 has secondary structural elements that include six β-sheets and five α-helices, wherein the β-sheets are designated B1-B6 and wherein the α-helices are designated H1-H5, and wherein the β-sheets and the α-helices connect with a B1-H1-B2-B3-H2-B4-H3-B5-H4-B6-H5 topology.

In another aspect, a method of using any of the crystals described above to screen for an agent that modulates Rab9 activity is provided, comprising the steps of: a) selecting a candidate agent by performing structure based drug design with the three-dimensional structure determined for the crystal, wherein selection is performed in conjunction with computer modeling; b) contacting the candidate agent with Rab9; and c) detecting the ability of the candidate agent to modulate Rab9. In another embodiment, the screening method comprises use of a candidate agent selected from a database, designed de novo, or designed from a known modulator of Rab9 activity. In yet another embodiment, the screening method comprises a candidate agent that is an inhibitor of Rab9, including competitive inhibitors and non-competitive or uncompetitive inhibitor of Rab9. In yet another embodiment, the step of employing the three-dimensional structure to design or select the candidate agent further comprises the steps of: a) screening for chemical entities or fragments capable of associating with Rab9; and b) assembling the chemical entities or fragments into a single molecule to provide the structure of the candidate agent.

In another embodiment, a method of screening for an agent that modulates Rab9 activity is provided, comprising the steps of: a) providing a model of Rab9 including at least one of the binding sites defined by at least one of Table 2, FIG. 4, FIG. 5, FIG. 6B, or FIG. 8; b) providing the structure of a candidate agent; and c) fitting the candidate agent to the target site, including determining the interactions between the candidate agent and at least one of the binding sites defined by at least one of Table 2, FIG. 4, FIG. 5, FIG. 6B, or FIG. 8. In another embodiment, the screening method further comprises the step of: d) selecting the fitted candidate agent. In yet another embodiment, the screening method further comprises the step of: e) contacting the candidate agent with Rab9 to determine the ability of the candidate agent to interact with Rab9. Alternatively, in yet another embodiment, the screening method further comprises the steps of: e) forming a complex of Rab9 and the candidate agent; and f) analyzing the complex to determine the ability of the candidate agent to interact with Rab9. In yet another embodiment, the screening method comprises use of X-ray crystallography or NMR spectroscopy.

In another embodiment or the screening method, the binding sites are selected from the group consisting of Region I, Region II, Region III, Region IV, Region V, Region VI, and Region VII of the C-terminally truncated human Rab9 defined by the amino acid sequence of SEQ ID NO:1, wherein Region I consists of amino acids 1 to 7 of SEQ ID NO:1, Region II consists of amino acids 33 to 43 of SEQ ID NO:1, Region III consists of amino acids 50 to 54 of SEQ ID NO:1, Region IV consists of amino acids 63 to 79 of SEQ ID NO:1, Region V consists of amino acids 108 to 117 of SEQ ID NO:1, Region VI consists of amino acids 128 to 130 of SEQ ID NO:1, and Region VII consists of amino acids 168 to 177 of SEQ ID NO:1. In yet another embodiment, the screening method screens for an agent that modulates Rab9 activity by inhibiting the binding of a Rab9-associated protein with Rab9, including but not limited to the Rab9-associated proteins TIP47 or P40.

In another embodiment, a method of screening for an agent that modulates Rab9 activity is provided, comprising the steps of: a) providing a three-dimensional structure of Rab9 as defined by the atomic coordinate data of Table 4 or Table 6, wherein said Rab9 comprises amino acids 1-177 of SEQ ID NO:1; b) employing the three-dimensional structure to design or select a candidate agent; c) synthesizing the candidate agent; and d) contacting the candidate agent with the Rab9 in the presence of a substrate to test the ability of the candidate agent to modulate the activity of the Rab9. In another embodiment, the screening method comprises use of a candidate agent selected from a database, designed de novo, or designed from a known modulator of Rab9 activity. In yet another embodiment, the screening method comprises a candidate agent that is an inhibitor of Rab9, including competitive inhibitors and non-competitive or uncompetitive inhibitor of Rab9. In yet another embodiment, the step of employing the three-dimensional structure to design or select the candidate agent further comprises the steps of: a) screening for chemical entities or fragments capable of associating with Rab9; and b) assembling the chemical entities or fragments into a single molecule to provide the structure of the candidate agent.

In another embodiment, a method of screening for an agent that modulates Rab9 activity is provided, comprising: a) selecting or designing a candidate agent by performing structure based drug design with a computer system encoded with computer readable data comprising atomic coordinate data or binding site data or both, wherein said atomic coordinate data is defined by Table 4 or Table 6, and wherein said binding site data is defined by at least one of Table 2, FIG. 4, FIG. 5, FIG. 6B, or FIG. 8, wherein said selecting is performed in conjunction with computer modeling; b) contacting the candidate agent with Rab9; and c) detecting the ability of the candidate agent to modulate Rab9.

In another embodiment, a computer system is provided, where the computer system is encoded with computer readable data comprising atomic coordinate data or binding site data or both, wherein said atomic coordinate data is defined by Table 4 or Table 6, and wherein said binding site data is defined by at least one of Table 2, FIG. 4, FIG. 5, FIG. 6B, or FIG. 8. In another embodiment, the computer system is encoded with binding site data selected from the group consisting of Region I, Region II, Region III, Region IV, Region V, Region VI, and Region VII of the C-terminally truncated human Rab9 defined by the amino acid sequence of SEQ ID NO:1, wherein Region I consists of amino acids 1 to 7 of SEQ ID NO:1, Region II consists of amino acids 33 to 43 of SEQ ID NO:1, Region III consists of amino acids 50 to 54 of SEQ ID NO:1, Region IV consists of amino acids 63 to 79 of SEQ ID NO:1, Region V consists of amino acids 108 to 117 of SEQ ID NO:1, Region VI consists of amino acids 128 to 130 of SEQ ID NO:1, and Region VII consists of amino acids 168 to 177 of SEQ ID NO:1. In another embodiment, the computer system is encoded with computer readable data comprising binding sites that mediate the binding of a Rab9-associated protein with Rab9, including but not limited to Rab9-associated proteins TIP47 or P40.

In another embodiment, a computer-readable medium is provided, where the computer-readable medium is encoded with atomic coordinate data or binding site data or both, wherein said atomic coordinate data is defined by Table 4 or Table 6, and wherein said binding site data is defined by at least one of Table 2, FIG. 4, FIG. 5, FIG. 6B, or FIG. 8. In another embodiment, the computer-readable medium is encoded with binding site data selected from the group consisting of Region I, Region II, Region III, Region IV, Region V, Region VI, and Region VII of the C-terminally truncated human Rab9 defined by the amino acid sequence of SEQ ID NO:1, wherein Region I consists of amino acids 1 to 7 of SEQ ID NO:1, Region II consists of amino acids 33 to 43 of SEQ ID NO:1, Region III consists of amino acids 50 to 54 of SEQ ID NO:1, Region IV consists of amino acids 63 to 79 of SEQ ID NO:1, Region V consists of amino acids 108 to 117 of SEQ ID NO:1, Region VI consists of amino acids 128 to 130 of SEQ ID NO:1, and Region VII consists of amino acids 168 to 177 of SEQ ID NO:1. In another embodiment, the computer-readable medium is encoded with computer readable data comprising binding sites that mediate the binding of a Rab9-associated protein with Rab9, including but not limited to Rab9-associated proteins TIP47 or P40. In another embodiment, a method of using the computer-readable medium is provided, wherein a graphical display software program is used to create an electronic file using the atomic coordinate data or the binding site data, where the electronic file can be visualized on a computer capable of representing said electronic file as a three dimensional image.

### BRIEF DESCRIPTION OF THE DRAWINGS

This patent contains multiple drawings executed in color. Copies of this patent with color drawings will be provided by the Office upon request and payment of the necessary fee.

FIG. 1 shows the amino acid sequence of human Rab9 and assignment of its secondary-structure elements. α-Helix and β-sheet regions defined by the crystal structure are underlined and labeled (α-helices starting with H and β-strands with B).

FIG. 2 shows a stereoview of the Cα trace of Rab9. Every 10th residue and both N and C termini are labeled. Residues 1, 35-38, and 176-177 are missing from the refined model and are not shown.

FIG. 3 shows a ribbon representation of Rab9 structure. A rainbow ramp color coding of blue to red is used to mimic chain trace from the N terminus to the C terminus. Both termini and the secondary structure elements are labeled. GDP in the active site is shown in ball-and-stick formation. Gray, carbon atoms; blue, nitrogen atoms; red, oxygen atoms; purple, phosphorus atoms.

FIG. 4 shows a stereoview of the active site showing the interactions between Rab9 and GDP. Gray, carbon atoms; blue, nitrogen atoms; red, oxygen atoms; purple, phosphorus atoms. The bonds in GDP are colored green.

FIG. 5 shows a structure-based sequence alignment of Rab9 with the most similar structures. The sequence number for the first residue of each line is indicated on the left. The positions of the secondary structural elements in Rab9 are indicated by underlining the sequence and labeling as in FIG. 1. Residues that are highly conserved in the Rab GTPase family are indicated in boldface type. Residues that are known to contact GDP in protein-GDP complex structures are indicated in red. Additional residues that would interact with the γ-phosphate in protein-GTP analog complex structures are shown in purple. The seven regions that show high degree of conformational variation among the superimposed structures (see FIG. 6) are highlighted in gold and labeled. See Table 3 for protein abbreviations and references.

FIG. 6 shows a structure comparison. A. A stereoview of structural alignment of Rab9 with three GTPases is shown. The Cα backbone in GDP-bound Rab9 (black, residues 2-34 and 39-175) is superimposed with GDP-bound Ypt7p (green, residues 7-37, 41-46, 77-181, and Protein Data Bank accession number 1KY3), GDP-bound Rab11a (blue, residues 6-173, and Protein Data Bank accession number 1OIV), and Gpp(NH)ₚ-bound p21Ras (red, residues 1-166, Protein Data Bank accession number 1CTQ). For clarity, only the GDP molecule in the active site of Rab9 structure is shown and colored black. Both N and C termini of Rab9 are labeled. B. A stereoview of Rab9 backbone structure is shown highlighting the seven hypervariable regions in yellow while the rest are shown in black. Both termini and the secondary structure elements are labeled. GDP in the active site is shown in ball-and-stick formation. Gray, carbon atoms; blue, nitrogen atoms; red, oxygen atoms; purple, phosphorus atoms.

FIG. 7 shows a stereo ribbon representation of Rab9-GppNHp complex structures. A. Monomer A′ is shown and depicts the “closed” conformation of Rab9. B. Monomer C′ is shown and depicts the “open conformation of Rab9. For both FIG. 7A and FIG. 7B, rainbow ramp color-coding of blue to red is used to mimic chain trace from the N-terminus to the C-terminus. Both termini and the secondary-structure elements are labelled α-helices starting with H and β-strands with B). GppNHp in the active site is shown in ball-and-stick. Gray, carbon atoms; blue, nitrogen atoms; red, oxygen atoms; purple, phosphorus atoms.

FIG. 8 shows a steroview of a structural comparison of Rab9-GppNHp with Rab9-GDP. The Cα backbone in GDP-bound Rab9 (blue, residues 2-34, 39-175) is superimposed with GppNHp-bound Rab9 monomer A′ (green, residues 6-175) and GppNHp-bound Rab9 monomer C′ (red, residues 6-174). The GDP/GNP (GppNHp) molecules in the active site of Rab9 structure are shown in sticks. Both termini and the secondary-structure elements are labeled.

### DETAILED DESCRIPTION OF THE INVENTION

The present invention now will be described more fully hereinafter with reference to the accompanying examples, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

The article “a” and “an” are used herein to refer to one or more than one (i.e., to at least one) of the grammatical object of the article. By way of example, “an element” means one or more than one element.

As used herein, sequences contain the one letter code for nucleotide sequence characters and the three letter codes for amino acids as defined in conformity with the IUPAC-IYUB standards described in Nucleic Acids Res. 13, 3021-3030 (1985) and in the Biochemical Journal 219, 345-373 (1984) which are herein incorporated by reference. Specifically, abbreviations of amino acids are defined below:

| | |
|---|---|
| A = Ala = alanine | T = Thr = threonine |
| V = Val = valine | C = Cys = cysteine |
| L = Leu = leucine | Y = Tyr = tyrosine |
| I = Ile = isoleucine | N = Asn = asparagine |
| P = Pro = proline | Q = Gln = glutamine |
| F = Phe = phenylalanine | D = Asp = aspartic acid |
| W = Trp = t4ryptophan | E = Glu = glutamic acid |
| M = Met = methionine | K = Lys = lysine |
| G = Gly = glycine | R = Arg = arginine |
| S = Ser = serine | H = His = histidine |

The terms “isolated” and “biologically pure” do not necessarily reflect the extent to which the protein has been purified. An isolated protein of the present invention can be obtained from its natural source, can be produced using recombinant DNA technology or can be produced by chemical synthesis. It is also to be noted that the terms “tertiary” and “three-dimensional” can be used interchangeably. It is also to be noted that reference to a “Rab9 protein” or a “Rab9 GTPase” can also be recited simply as “Rab9” and such terms can be used to refer to the complete Rab9 protein, a portion of the Rab9 protein, such as a polypeptide, and/or a monomer or a dimer of the Rab9 protein. When reference is specifically made to a monomer or dimer, for example, such term is typically used in conjunction with the Rab9 protein name.

The term “unit cell” refers to a basic parallelepiped-shaped block (in other words, a six faced block, each a parallelogram and each being parallel to the opposite face). The entire volume of a crystal may be constructed by regular assembly of such blocks. Each unit cell comprises a complete representation of the unit of pattern, the repetition of which builds up the crystal.

The term “space group” refers to the arrangement of symmetry elements of a crystal.

The term “root mean square deviation” refers to the square root of the arithmetic mean of the squares of the deviations from the mean.

The term “complex” refers to a Rab9 protein (or Rab9 truncation or homologue) in covalent or non-covalent association with a ligand. Ligand may include, for example, a chemical entity, compound, or inhibitor, candidate drug, and the like. The term “association” refers to a condition of proximity between the ligand and Rab9, or their respective portions thereof, in any appropriate physicochemical interaction. Ligands may include, but are not limited to, GDP, GTP, and GTP analog Guanosine 5′-(β,γ-imido) triphosphate (GppNHp).

The term “binding site” refers to a site, such as an atom or functional group of an amino acid residue, in the Rab9 active site that may bind to an agent or Rab9-associated protein. The term “active site” refers to any or all of the following: 1) the portion of the Rab9 sequence that binds to a substrate; 2) the portion of the Rab9 sequence that binds to an inhibitor; 3) the portion of the Rab9 sequence that binds to GDP, GTP, and/or GppNHp; and 4) the portion of the Rab9 sequence that binds to a Rab9-associated protein. Depending on the particular molecule bound to Rab9, sites may exhibit attractive or repulsive binding interactions, brought about by charge, steric considerations and the like.

The term “Rab9-associated protein” refers to a protein that normally interacts with Rab9, including but not limited to such Rab9 interacting proteins as Rab effector P40 and the vesicle cargo selection protein TIP47.

By “fitting” is meant determining by automatic, or semi-automatic means, interactions between one or more atoms of an agent and one or more binding sites of Rab9, and determining the extent to which such interactions are stable. Various computer-based methods for fitting are described further herein.

By a “computer system” is meant the hardware means, software means and data storage means used to analyze atomic coordinate data. The computer may comprise a central processing unit (“CPU”), a working memory, for example, random access memory (“RAM”) and/or storage memory in the form of one or more disk drives (e.g., floppy, Zip™, Jazz™), tape drives, CD-ROM drives, DVD drives, and the like, a display terminal such as for example, a cathode ray tube type display, and input and output lines for data transmission, including a keyboard and/or mouse controller. The computer may be a stand-alone, or connected to a network and/or shared server. Examples of such systems are microcomputer workstations available from Silicon Graphics Incorporated and Sun Microsystems running Unix based, Windows NT or IBM OS/2 operating systems.

By “computer readable media” or “CRM” is meant any media which can be read and accessed directly by a computer, for example so that the media is suitable for use in the above-mentioned computer system. Computer-readable data storage materials include, for example, floppy discs, hard disc storage medium and magnetic tape; optical storage media such as optical discs or CD-ROM; electrical storage media such as RAM and ROM; Zip™ and Jazz™-type disks; tapes; CDs; and DVDs; and hybrids of these categories such as magnetic/optical storage media.

Rab Proteins, Rab9 GTPase, and Rab9-Associated Proteins

Rab proteins, the largest subfamily of the Ras-like small GTPase superfamily, serve as molecular switches mediating tethering, docking, fusion, and motility of intracellular membranes (Pfeffer, S. R. (2001) Trends Cell Biol. 11, 487-491). Rabs cycle between GTP-bound (on/active) and GDP-bound (off/inactive) conformations (Pfeffer, S. R. (1994) Curr. Opin. Cell Biol. 6, 522-526; Bourne, H. R. et al. (1991) Nature 349, 117-127; Sprang, S. R. (1997) Annu. Rev. Biochem. 66, 639-678). The active form is stabilized by additional hydrogen bond interactions with the γ-phosphate of GTP mediated by serine residues in the phosphate-binding loop and switch I region as well as an extensive hydrophobic interface between the switch I and II regions (Dumas, J. J. et al. (1999) Structure Fold Des. 7, 413-423; Esters, H. et al. (2000) J. Mol. Biol. 298, 111-121). The inactive conformation usually has displaced and mobile switch regions (Bourne, H. R. et al. (1991) Nature 349, 117-127; Stroupe, C., and Brunger, A. T. (2000) J. Mol. Biol. 304, 585-598). Biochemical and genetic studies of chimeric and mutant Rab proteins have identified several hypervariable regions, including the N and C termini and the α3/β5 loop, that play an important role in determining functional specificity (Brennwald, P., and Novick, P. (1993) Nature 362, 560-563; Stenmark, H. et al. (1994) EMBO J. 13, 575-583).

The Rab9 GTPase is localized predominantly to late endosomes and is required for the transport of mannose 6-phosphate receptors from endosomes to the trans-Golgi network (Lombardi, D. et al. (1993) EMBO J. 12, 677-682; Riederer, M. A. et al. (1994) J. Cell Biol. 125, 573-582). By targeting Rab9 mRNA for degradation with small interfering RNA, Rab9 has been identified to be a key cellular component for HIV-1, Ebola, Marburg, and measles virus replication, suggesting that inhibitors of Rab9 function, if developed, might prove useful in the control of those viruses.

Rab9-associated proteins may be involved in the normal physiological activity of Rab9. For example, Rab9 facilitates vesicular transport by pairing with its cognate Rab effector P40 (Diaz, E. et al. (1997) J. Cell Biol. 138, 283-290). Rab9 also interacts with the vesicle cargo selection protein TIP47, which has been shown to bind the cytoplasmic tail of the HIV envelope glycoprotein subunit gp41 (Blot, G. et al. (2003) J. Virol. 77, 6931-6945). Because Rab9-associated proteins may be involved in the normal physiological activity of Rab9, in one embodiment, binding sites mediating the interaction of Rab9 with its associated proteins are provided as targets for structure-based drug design and development.

In one embodiment of the present invention, a crystal of a C-terminally truncated human Rab9 having the three dimensional atomic coordinates of Table 4 or Table 6, wherein said Rab9 comprises amino acids 1-177 of SEQ ID NO:1.

As discussed herein, the term “Rab9” in intended to to include all or part of the amino acid sequence of SEQ ID NO:1 as well as any biologically active variants of human Rab9 in which functionally equivalent amino acid residues are substituted for residues within the sequence resulting in conservative amino acid substitutions. A “biologically active variant” of human Rab9 is a polypeptide derived from the human Rab9 polypeptide by deletion (so-called truncation) or addition of one or more amino acids to the N-terminal and/or C-terminal end of the native protein; deletion or addition of one or more amino acids at one or more sites in the protein; or substitution of one or more amino acids at one or more sites in the protein. Biologically active variant human Rab9 polypeptides encompassed by the present invention are biologically active, that is they are capable of having the GTPase activity of the Rab protein subfamily of the Ras-like small GTPase superfamily (Lombardi et al. (1993) EMBO J. 12: 677-82). Such biologically active variants may result from, for example, genetic polymorphism or from human manipulation. Biologically active variants of human Rab9 according to the invention will have at least about 50%, 60%, 65%, 70%, generally at least about 75%, 80%, 85%, preferably at least about 90%, 91%, 92%, 93%, 94%, 95%, 96%, 97%, such as at least about 98%, 99% or more sequence identity to the amino acid sequence shown in SEQ ID NO:1. Thus, a biologically active variant of human Rab9 of the invention may differ from the amino acid sequences shown in SEQ ID NO:1 by as few as 1-15 amino acid residues, as few as 1-10 amino acid residues, such as 6-10 amino acid residues, as few as 5 amino acid residues, or as few as 4, 3, 2, or even 1 amino acid residue.

In order to retain biological activity, any substitutions will preferably be conservative in nature, and truncations and substitutions will generally made in residues that are not required for GTPase activity. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. Polar neutral amino acids include glycine, serine, threonine, cysteine, tyrosine, asparagine, and glutamine. Positively charged (basic) amino acids include arginine, lysine, and histidine. Negatively charged (acidic) amino acids include aspartic acid and glutamic acid. Such alterations will not be expected to affect apparent molecular weight as determined by polyacrylamide gel electrophoresis, or isoelectric point.

The comparison of sequences and determination of percent identity and percent similarity between two sequences can be accomplished using a mathematical algorithm. In a preferred embodiment, the percent identity between two amino acid sequences is determined using the Needleman and Wunsch (1970) J. Mol. Biol. 48:444-453 algorithm, which is incorporated into the GAP program in the GCG software package (available at www.accelrys.com), using either a BLOSSUM62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a BLOSUM62 scoring matrix (see Henikoff et al. (1989) Proc. Natl. Acad. Sci. USA 89:10915) and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity limitation of the invention) is using a BLOSUM62 scoring matrix with a gap weight of 60 and a length weight of 3).

The percent identity between two amino acid or nucleotide sequences can also be determined using the algorithm of E. Meyers and W. Miller (1989) CABIOS 4:11-17 which has been incorporated into the ALIGN program (version 2.0), using a PAM120 weight residue table, a gap length penalty of 12 and a gap penalty of 4.

In another embodiment, a crystal of a Rab9-GDP complex is provided wherein the crystal effectively diffracts X-rays for the determination of the atomic coordinates of the Rab9-GDP complex to a resolution of less than about 1.75 Å, preferably between about 1.25 Å and about 1.75 Å, and most preferably about 1.25 Å. In another embodiment, a crystal of a C-terminally truncated human Rab9 (residues 1-177) is provided having a space group of P₁ and a unit cell of dimensions a=38.40 Å, b=45.62 Å, c=51.22 Å, α=99.80, β=107.2°, and γ=101.8°. In another embodiment, a crystal of a C-terminally truncated human Rab9 (residues 1-177) is provided where the Rab9 has secondary structural elements that include six β-sheets and five α-helices, where the β-sheets are designated B1-B6 and where the α-helices are designated H1-H5, and where the β-sheets and the α-helices connect with a B1-H1-B2-B3-H2-B4-H3-B5-H4-B6-H5 topology.

In another embodiment, a crystal of a Rab9-GppNHp complex is provided wherein the crystal effectively diffracts X-rays for the determination of the atomic coordinates of the Rab9-GppNHp complex to a resolution of less than about 20 Å, preferably less than about 2 Å, more preferably between about 1.25 Å and about 2 Å, and most preferably about 1.73 Å. In another embodiment, a crystal of a Rab9-GppNHp complex is provided having a space group of P2₁2₁2, and a unit cell of dimensions a=56.24 Å, b=76.60 Å, and c=174.35 Å. In another embodiment, Rab9-GppNHp complex is provided wherein the Rab9 has secondary structural elements that include six β-sheets and five α-helices, wherein the β-sheets are designated B1-B6 and wherein the α-helices are designated H1-H5, and wherein the 1-sheets and the α-helices connect with a B1-H1-B2-B3-H2-B4-H3-B5-H4-B6-H5 topology.

Structure Based Drug Design

Structure based drug design involves the rational design of ligand molecules to interact with the three-dimensional (“3-D”) structure of target receptors; the ultimate goal being to identify or design molecules with 3-D complementarity to the target protein (Kirkpatrick et al. (1999) Comb. Chem. High Throughput Screen. 2: 211-21). The accuracy required of a protein structure depends on the question addressed by the design process, with some processes predicated on the assumption that a lead molecule will need to complement a known binding site for a ligand precisely, or match the presumed transition state structure of a reaction closely (Whittle and Blundell (1994) Annu. Rev. Biophys. Biomol. Struct. 23: 349-75). Such cases call for an accurate model at the highest resolution possible. Alternatively, the design process may exploit the structure to indicate the general availability of space to fill, hydrogen bonds to make, or electrostatic interactions to optimize, in which case knowledge of the general topography of the binding site is often useful (Whittle and Blundell (1994) Annu. Rev. Biophys. Biomol. Struct. 23: 349-75).

Factors that affect the accuracy of structure-based drug design include aspects of the determination of the three-dimensional structure of proteins such as refinement, resolution, the number of restraints introduced in the structure analysis, statistical indicators of agreement between the model and the experimental data, and the conformity of the model to stereochemistry found in proteins in general (Whittle and Blundell (1994) Annu. Rev. Biophys. Biomol. Struct. 23: 349-75). Most statistical parameters can be optimized, at least within the constraints of the data. However, if the data is of poor quality or the conformations are incorrect (particularly for the sidechains and loops), then it is difficult to optimize all of the parameters at the same time (Whittle and Blundell (1994) Annu. Rev. Biophys. Biomol. Struct. 23: 349-75). Computer programs are available to introduce a check on such parameters, including PROCHECK™, which analyzes the distribution of a range of conformational parameters and compares them with expected distributions (Laskowski et al. (1993). J. Appl. Crystallogr. 26:283). Sequence-dependent indications of the probability that the structure is correct can be derived through a comparison of the local environment in the proposed structure to the propensity of an amino acid (Luthy et al. (1991) Proteins Struct. Funct. Genet. 10: 229; Novotny et al. (1988) Proteins Struct. Funct. Genet. 4: 19), the knowledge-based potential (Hendlich et al. (1990) J. Mol. Biol. 216: 167), or the probability of amino acid substitution (Overington et al. (1990) Proc. R. Soc. London Ser. B 241: 132; Topham et al. (1991) Biochem. Soc. Syrup. 57: 1) in the proposed structure.

Protein structures cannot generally be predicted by simulation of the folding pathway due to the fact that the forces between the atoms of the protein, and particularly with the surrounding solvent and counter-ions, are not very well described (Whittle and Blundell (1994) Annu. Rev. Biophys. Biomol. Struct. 23: 349-75). However, proteins belong to families with a common fold, including more than 1500 groups of homologous proteins that can be recognized by sequence searches alone, and over 500 that have common topologies or folds (Whittle and Blundell (1994) Annu. Rev. Biophys. Biomol. Struct. 23: 349-75).

Profiles or templates are useful in the search for the common fold and alignment of sequences for proteins with sequence identities of <30% (Whittle and Blundell (1994) Annu. Rev. Biophys. Biomol. Struct. 23: 349-75). Structural information can be used to identify key features in protein architecture and then to associate these with invariant or conserved sequences (Bedarkar et al. (1977) Nature, 270: 449; Eigenbrot et al. (1991) J. Mol. Biol. 221; 15). Projection of the restraints of the three-dimensional fold onto the one dimension of the sequence and comparison to sequence templates or profiles provides a more systematic approach (Sali et al. (1990) J. Mol. Biol. 212: 403). The template search can also be approached by determining the propensity of an amino acid to occur in each class of local structural environment defined by solvent accessibility and secondary structure, or by calculation of amino acid substitution tables as a function of local environment (Bowie et al. (1991) Science 253: 164; Johnson et al. (1993) J. Mol. Biol. 231: 735; Luthy et al. (1991) Proteins Struct. Funct. Genet. 10: 229; Overington et al. (1990) Proc. R. Soc. London Ser. B 241: 132).

The three-dimensional structure of a protein can also be predicted by using information derived from the identification of a new sequence with a known fold (Summers et al. (1987) J. Mol. Biol. 196: 175; Sutcliffe et al. (1987) Protein Eng. 1: 385). Some methods depend on the assembly of rigid fragments to select sets of fragments that define the framework: the structurally variable (mainly loop) regions and the sidechains (Blundell et al. (1988) Ear. J. Biochem. 172: 513; Blundell et al. (1987) Nature 326: 347; Claessens et al. (1989) Protein Eng. 2: 335; Jones et al. EMBO J. 5: 819; Topham et al. (1993) J. Mol. Biol. 229: 194). Such modeling procedures are very successful when the percentage sequence identity to the unknown is high (greater than 40%) and when the known structures cluster around that to be predicted (Srinivasen & Blundell (1993) Protein Eng. 6: 501).

Where a common fold is not known, combinatorial approaches that depend upon the identification of secondary-structure elements using conformational propensities and residue patterns can be valuable (Presnell et al. (1992) Biochemistry 31: 983). The elements of secondary structure are then assembled by docking and/or by using rules concerning supersecondary structures (Whittle and Blundell (1994) Annu. Rev. Biophys. Biomol. Struct. 23: 349-75).

The present invention provides a method of using crystals of a human Rab9 as described herein (for example, a crystal of a Rab9-GDP complex or a Rab9-GppNHp complex, or both) to screen for an agent that modulates Rab9 activity. The screening method of the present invention may comprise use of a candidate agent selected from a database, designed de novo, or designed from a known modulator of Rab9 activity. Suitable candidate agents of the present invention include peptides or other organic molecules, and inorganic molecules. Suitable organic molecules include small organic molecules. Preferably, a therapeutic compound of the present invention is not harmful (e.g., toxic) to an animal when such compound is administered to an animal. Peptides refer to a class of compounds that is small in molecular weight and yields two or more amino acids upon hydrolysis. A polypeptide is comprised of two or more peptides. As used herein, a protein is comprised of one or more polypeptides. Preferred therapeutic compounds to design include peptides composed of “L” and/or “D” amino acids that are configured as normal or retroinverso peptides, peptidomimetic compounds, small organic molecules, or homo- or hetero-polymers thereof, in linear or branched configurations.

In one embodiment, the screening method of the present invention comprises the steps of: a) selecting a candidate agent by performing structure based drug design with the three-dimensional structure determined for crystals of a human Rab9 as described herein (for example, a crystal of a Rab9-GDP complex or a Rab9-GppNHp complex, or both), where selection is performed in conjunction with computer modeling; b) contacting the candidate agent with Rab9; and c) detecting the ability of the candidate agent to modulate Rab9. The step of employing the three-dimensional structure to design or select the candidate agent may further comprise the steps of: a) screening for chemical entities or fragments capable of associating with Rab9; and b) assembling the chemical entities or fragments into a single molecule to provide the structure of the candidate agent.

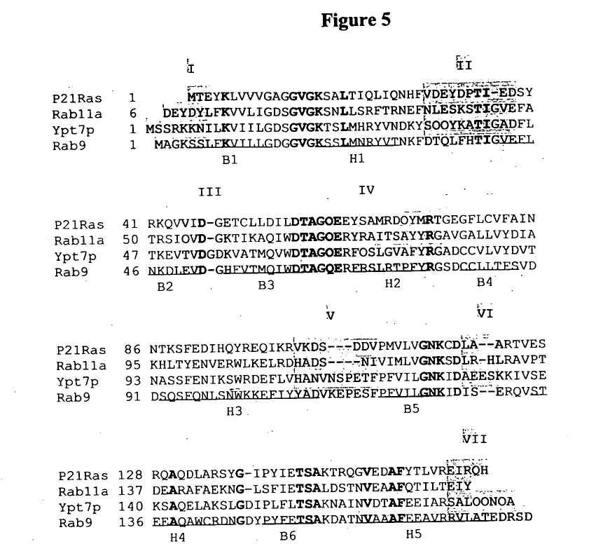

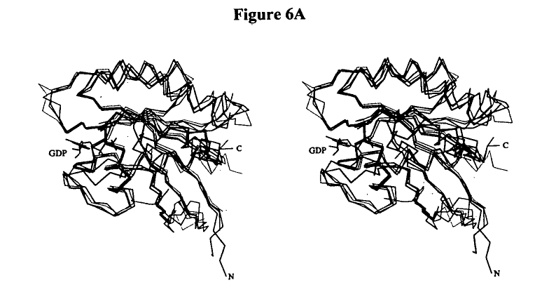

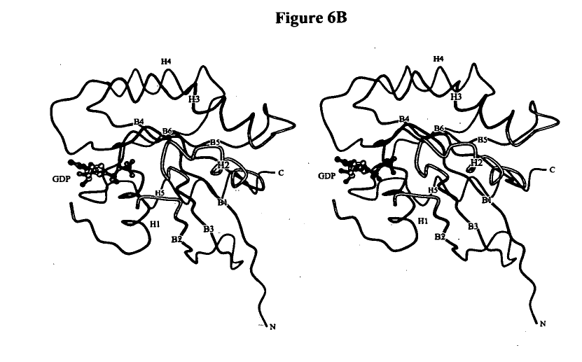

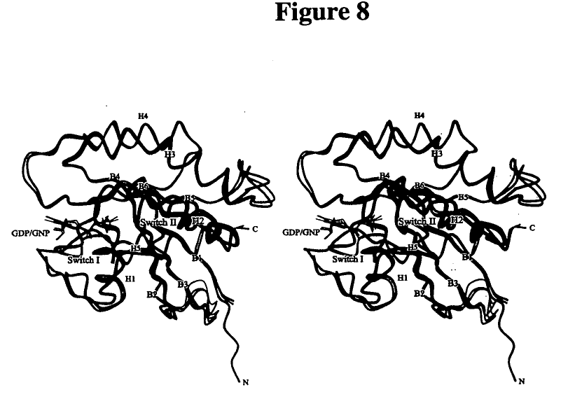

In another embodiment, the screening method of the present invention comprises the steps of: a) providing a model of Rab9, said model including at least one of the binding sites defined by at least one of Table 2, FIG. 4, FIG. 5, FIG. 6B, or FIG. 8; b) providing the structure of a candidate agent; and c) fitting the candidate agent to said target site, including determining the interactions between the candidate agent and at least one of the binding sites defined by at least one of Table 2, FIG. 4, FIG. 5, FIG. 6B, or FIG. 8. The screening method may further comprise the step of: d) selecting the fitted candidate agent. The screening method may even further comprise the step of: e) contacting the candidate agent with Rab9 to determine the ability of the candidate agent to interact with Rab9. Alternatively, the screening method may even further comprise the step of: e) forming a complex of Rab9 and the candidate agent; and f) analyzing the complex to determine the ability of the candidate agent to interact with Rab9. Analysis of the complex to determine the ability of the candidate agent to interact with Rab9 may be performed by any means known in the art, including but not limited to X-ray crystallography or NMR spectroscopy.

Rab9 contains seven hypervariable regions that are significantly different in conformation from other Rab proteins. These seven hypervariable regions in Rab9 structure are thought to be involved in the binding of associated proteins to Rab9. Without being bound by theory, because Rab9-associated proteins may be involved in the normal physiological activity of Rab9, these seven hypervariable regions in Rab9 provide an excellent target for structure-based drug design and development.

In another embodiment, the screening method of the present invention provides a model of Rab9 including binding sites selected from the group consisting of Region I, Region II, Region III, Region IV, Region V, Region VI, and Region VII of the C-terminally truncated human Rab9 defined by the amino acid sequence of SEQ ID NO:1, wherein Region I consists of amino acids 1 to 7 of SEQ ID NO:1, Region II consists of amino acids 33 to 43 of SEQ ID NO:1, Region III consists of amino acids 50 to 54 of SEQ ID NO:1, Region IV consists of amino acids 63 to 79 of SEQ ID NO:1, Region V consists of amino acids 108 to 117 of SEQ ID NO:1, Region VI consists of amino acids 128 to 130 of SEQ ID NO:1, and Region VII consists of amino acids 168 to 177 of SEQ ID NO:1. In yet another embodiment, the screening method screens for an agent that modulates Rab9 activity by inhibiting the binding of a Rab9-associated protein with Rab9, including but not limited to wherein the Rab9-associated protein is TIP47 or P40.

In another embodiment, the screening method of the present invention screens for an agent that modulates Rab9 activity, and comprises the steps of: a) providing a three-dimensional structure of Rab9 as defined by the atomic coordinate data of Table 4 or Table 6, wherein said Rab9 comprises amino acids 1-177 of SEQ ID NO:1; b) employing the three-dimensional structure to design or select a candidate agent; c) synthesizing the candidate agent; and d) contacting the candidate agent with the Rab9 in the presence of a substrate to test the ability of the candidate agent to modulate the activity of the Rab9. The step of employing the three-dimensional structure to design or select the candidate agent may further comprise the steps of: a) screening for chemical entities or fragments capable of associating with Rab9; and b) assembling the chemical entities or fragments into a single molecule to provide the structure of the candidate agent.

In another embodiment, the screening method of the present invention screens for an agent that modulates Rab9 activity, and comprises the steps of: a) selecting or designing a candidate agent by performing structure based drug design with a computer system encoded with computer readable data comprising atomic coordinate data or binding site data or both, wherein said atomic coordinate data is defined by Table 4 or Table 6, and wherein said binding site data is defined by at least one of Table 2, FIG. 4, FIG. 5, FIG. 6B, or FIG. 8, wherein said selecting is performed in conjunction with computer modeling; b) contacting the candidate agent with Rab9; and c) detecting the ability of the candidate agent to modulate Rab9.

In one embodiment of the present invention, the screening method comprises a candidate agent that is an inhibitor of Rab9, including competitive inhibitors and non-competitive or uncompetitive inhibitor of Rab9. A potential inhibitor is selected by performing rational drug design with the three-dimensional structure (or structures) determined for a crystal as described herein, especially in conjunction with computer modeling and methods as described herein. The potential inhibitor is obtained from commercial sources or is synthesized from readily available starting materials using standard synthetic techniques and methodologies known to those of ordinary skill in the art. The potential inhibitor is then assayed to determine its ability to inhibit Rab9 and/or a Rab9-associated intracellular process or pathway. The assay may be in vitro or in vivo. Inhibition can be measured by various methods, including, for example, those methods illustrated in the examples below. Assays include any assay wherein a nucleoside or nucleotide are cofactors or substrates of the peptide of interest, and particularly any assay involving phosphotransfer in which the substrates and or cofactors are GDP, GTP, and/or GppNHp. The assay may be an enzyme inhibition assay, utilizing a full length or truncated GTPase. The enzyme is contacted with the potential inhibitor and a measurement of the binding affinity of the potential inhibitor against a standard is determined. Such assays are known to one of ordinary skill in the art. The assay may also be a cell-based assay in which the potential inhibitor is contacted with a cell and a measurement of inhibition of a standard marker produced in the cell is determined. Cells may be either isolated from an animal, including a transformed cultured cell, or may be in a living animal. Such assays are also known to one of ordinary skill in the art.

A variety of methods are available to one skilled in the art for evaluating and virtually screening candidate agents appropriate for associating with Rab9. Such association may be in a variety of forms including, for example, steric interactions, van der Waals interactions, electrostatic interactions, solvation interactions, charge interactions, covalent bonding interactions, non-covalent bonding interactions (e.g., hydrogen-bonding interactions), entropically or enthalpically favorable interactions, and the like.

Computational Approaches to Structure Based Drug Design

Once the three-dimensional structure of a target protein has been defined, computational procedures may be used to suggest ligands that will bind at the active site. Interactive graphics approaches explore new ligand designs manually in ways that might involve, for example, modification of groups on the ligand to optimize complementarity with receptor/enzyme subsites, optimization of a transition state isostere to reflect data from mechanistic studies, replacement of peptide bonds with groups that improve hydrolytic stability while maintaining key hydrogen bond interactions, or linking of adjacent side groups to increase the rigidity of the ligand (Whittle and Blundell (1994) Annu. Rev. Biophys. Biomol. Struct. 23: 349-75). Most of these steps can now be done using systematic computational approaches that fall into three classes: 1) automated docking of whole molecules into receptor sites; 2) precalculating potentials at grid points and fitting molecules to these potentials; and 3) docking fragments and either joining them or growing them into real molecules (Whittle and Blundell (1994) Annu. Rev. Biophys. Biomol. Struct. 23: 349-75).

Attempts at automated docking through the evaluation of electrostatic, steric, or more complex energy terms during a systematic search of rotational and translational space for the two molecules have produced some successes, but the simplification of energy functions required to achieve reasonable computational times has proved limiting (Kuntz et al. (1982). J. Mol. Biol. 161: 269; Wodak (1978) J. Mol. Biol. 124: 323; Whittle and Blundell (1994) Annu. Rev. Biophys. Biomol. Struct. 23: 349-75). Interactive or manual docking involving the positioning of molecules with constant feedback of the energy has been used as an alternative, but the many degrees of freedom and modes of interaction, however, have imposed their own limitations on the utility of this approach (Busetta et al. (1983) J. Appl. Crystallogr. 16: 432; Pattabiraman et al. (1985) J. Comput. Chem. 6: 432; Tomioka et al. (1987) J. Comput. Aided Mol. Des. 1: 197).

Precalculating terms for each point on a grid can be used to identify hydrogen-bonding sites within enzyme active sites and also significantly reduces computational time (Goodford (1985) J. Med. Chem. 28: 849). A similar approach involves the use of pseudoenergies calculated from pairwise distributions of atoms in protein complexes or crystals of small molecules, with probe molecules then fitted to these potentials and ranked according to energy (Whittle and Blundell (1994) Annu. Rev. Biophys. Biomol. Struct. 23: 349-75). For example, software such as DOCK (available from University of California, San Francisco), creates a negative image of the target site by placing a set of overlapping spheres so that they fill the complex invaginations of the proposed binding site, and the putative ligands are then placed into the site by matching X-ray or computer derived structures on the basis of a comparison of internal distances (Whittle and Blundell (1994) Annu. Rev. Biophys. Biomol. Struct. 23: 349-75). The candidates are then ranked on the basis of their best orientations. Other methods include a directed version of DOCK that allows for hydrogen-bond information to be used and conformational flexibility to be allowed, and a method that uses least squares fitting to maximize overlap of enzymes and putative ligands (Leach & Kuntz (1992) J. Comput. Chem. 13: 730; Bacon & Moult (1992) J. Mol. Biol. 225: 849). Still further methods involve the use of genetic algorithms and graph theory to generate molecular structures within constraints of an enzyme active site or a receptor binding site (Payne & Glen (1993) J. Mol. Graph. 11: 76; Lewis (1993) J. Mol. Graph. 10: 131). For all of these methods to be useful in drug discovery, however, they must depend upon the existence of large data bases of small molecule structures, such as the Cambridge Structure Data Base and the Fine Chemicals Directory (Allen et al. (1979) Acta Cryst. B 35: 2331; Rusinko et al. (1989) J. Chem. Inf. Comput. Sci. 29: 251).

Methods involving fragment docking and then developing algorithms to grow them into larger structures to fill the space available depend upon the exploration of electrostatic, van der Waals, or hydrogen bonding interactions involved in molecular recognition (Whittle and Blundell (1994) Annu. Rev. Biophys. Biomol. Struct. 23: 349-75). Many of these methods incorporate the GRID algorithm as a starting point, and then use GenStar and/or GroupBuild to generate chemically reasonable structures to fill the active sites of enzymes (Rotstein and Murcko (1993) J. Comput. Aided Mol. Des. 7: 23; Rotstein and Murcko (1993) J. Med. Chem. 36: 1700). Alternatively, the program can start with a docked core or the structure of a fragment from an inhibitor complex and for each atom generated, several hundred candidate positions, representing different bond lengths and torsion angles, are scored on the basis of contacts with the enzyme (Whittle and Blundell (1994) Annu. Rev. Biophys. Biomol. Struct. 23: 349-75).

Numerous computer programs are available and suitable for rational drug design and the processes of computer modeling, model building, and computationally identifying, selecting and evaluating potential inhibitors in the methods described herein. These include, for example, SYBYL (available from TRIPOS, St. Louis Mo.), DOCK (available from University of California, San Francisco), GRID (available form Oxford University, UK), MCSS (available from Molecular Simulations Inc., Burlington, Mass.), AUTODOCK (available from Oxford Molecular Group), FLEX X (available from TRIPOS, St. Louis Mo.), CAVEAT (available from University of California, Berkeley), HOOK (available from Molecular Simulations Inc., Burlington, Mass.), and 3-D database systems such as MACCS-3D (available from MDL Information Systems, San Leandro, Calif.), UNITY (available from TRIPOS, St. Louis Mo.), and CATALYST (available from Molecular Simulations Inc., Burlington, Mass.). Potential inhibitors may also be computationally designed de novo using such software packages as LUDI (available from Biosym TechMA), and LEAPFROG (TRIPOS Associates, St. Louis, Mo.). Compound deformation energy and electrostatic repulsion, may be evaluated using programs such as GAUSSIAN 92, AMBER, QUANTA/CHARMM, and INSIGHT II/DISCOVER. These computer evaluation and modeling techniques may be performed on any suitable hardware including for example, workstations available from Silicon Graphics, Sun Microsystems, and the like. These techniques, methods, hardware and software packages are representative and are not intended to be comprehensive listing. Other modeling techniques known in the art may also be employed in accordance with this invention. See for example, N. C. Cohen, Molecular Modeling in Drug Design, Academic Press (1996); Whittle and Blundell (1994) Annu. Rev. Biophys. Biomol. Struct. 23: 349-75; Grootenhuis et al. (1992) Bull. Soc. Chim. Belg. 101: 661; Lawrence and Davis (1992) Proteins Struct. Funct. Genet. 12: 31; Miranker and Karplus (1991) Proteins Struct. Funct. Genet. 11: 29). Other methods and programs include CLIX (a suite of computer programs that searches the Cambridge Data base for small molecules that have both geometrical and chemical complementarity to a defined binding site on a protein of known three-dimensional structure), and software identified at internet sites including the CAOS/CAMM Center Cheminformatics Suite at http://www.caos.kun.nl/, and the NIH Molecular Modeling Home Page at http://www.fi.muni.cz/usr/mejzlik/mirrors/molbio.info.nih.gov/modeling/software list/.

In one embodiment of the present invention a computer system is provided, wherein the computer system is encoded with computer readable data comprising atomic coordinate data or binding site data or both, wherein said atomic coordinate data is defined by Table 4 or Table 6, and wherein said binding site data is defined by at least one of Table 2, FIG. 4, FIG. 5, FIG. 6B, or FIG. 8. In another embodiment, the computer system is encoded with binding site data selected from the group consisting of Region I, Region II, Region III, Region IV, Region V, Region VI, and Region VII of a C-terminally truncated human Rab9 as defined by the amino acid sequence of SEQ ID NO:1, wherein Region I consists of amino acids 1 to 7 of SEQ ID NO:1 (MAGKSSL), Region II consists of amino acids 33 to 43 of SEQ ID NO:1 (DTQLFHTIGVE), Region III consists of amino acids 50 to 54 of SEQ ID NO:1 (EVDGH), Region IV consists of amino acids 63 to 79 of SEQ ID NO:1 (TAGQERFRSLRTPFYRG), Region V consists of amino acids 108 to 117 of SEQ ID NO:1 (YADVKEPESF), Region VI consists of amino acids 128 to 130 of SEQ ID NO:1 (ISE), and Region VII consists of amino acids 168 to 177 of SEQ ID NO:1 (RVLATEDRSD). In another embodiment, the computer system is encoded with computer readable data comprising binding sites that mediate the binding of a Rab9-associated protein with Rab9, including but not limited to Rab9-associated proteins TIP47 or P40.

In another embodiment, a computer-readable medium is provided, where the computer-readable medium is encoded with atomic coordinate data or binding site data or both, wherein said atomic coordinate data is defined by Table 4 or Table 6, and wherein said binding site data is defined by at least one of Table 2, FIG. 4, FIG. 5, FIG. 6B, or FIG. 8. In another embodiment, the computer-readable medium is encoded with binding site data selected from the group consisting of Region I, Region II, Region III, Region IV, Region V, Region VI, and Region VII of a C-terminally truncated human Rab9 (residues 1-177), as described above. In another embodiment, the computer-readable medium is encoded with computer readable data comprising binding sites that mediate the binding of a Rab9-associated protein with Rab9, including but not limited to Rab9-associated proteins TIP47 or P40. In another embodiment, a method of using the computer-readable medium is provided, wherein a graphical display software program is used to create an electronic file using the atomic coordinate data or the binding site data, where the electronic file can be visualized on a computer capable of representing the electronic file as a three dimensional image.

### EXPERIMENTAL

### Example 1

### Crystal Structure of Human Rab9-GDP Complex

For this experiment, the crystal structure of a C-terminally truncated human Rab9 (residues 1-177) in complex with GDP (Rab9-GDP) was deterimined.

Methods

Cloning and Expression—The gene for human Rab9 (GenBank™ accession number NM_{—}004251) was obtained from the IMAGE clone collection (IMAGE ID number 4139714) through distribution by Open Biosystems. A C-terminally truncated fragment coding for residues 1-177 (20.1 kDa) was PCR subcloned using primers 5′-G ACA GCT AGC ATG GCA GGC AAA TCA TCA CTT TTT AAA G-3′ and 5′-C ATG GAT CCT TCA GTC CTC GGT AGC AAG AAC TCT TC-3′ into the NheI/BamHI restriction sites of pET28b (Novagen); the resulting construct encodes for a Rab9-(1-177) protein product with an N-terminal His6-containing fusion (MGSSHHHHHHSSGLVPRGSHMAS). The pET28-Rab9-(1-177) vector was transformed into Escherichia coli BL21(DE3) (Novagen), and overproduction of the fusion protein was induced at an A_{600 nm} of ˜2.0 with 1 mM isopropyl-1-thio-β-D-galactopyranoside at 310 K for 2 h; the cells were harvested by centrifugation and frozen at 253 K.

Protein Purification—The cell pellet was re-suspended in nickel buffer A (20 mM Tris, pH 8.0, 500 mM NaCl, 5 mM imidazole), lysed by sonication, and centrifuged at 20,000×g for 20 min at 277 K. The soluble fraction was filtered through a 0.45-micron filter and applied to chelating Sepharose (Amersham Biosciences), which had been previously charged with 50 mM NiSO4 and equilibrated with nickel buffer A. The column was then washed with nickel wash buffer (20 mM Tris-HCl, pH 8.0, 500 mM NaCl, 55 mM imidazole), and the His6-Rab9-(1-177) fusion protein was eluted with nickel elution buffer (20 mM Tris-HCl, pH 8.0, 500 mM NaCl, 350 mM imidazole). The His6-Rab9-(1-177) fusion protein was then dialyzed against Thr buffer (20 mM Tris-HCl, pH 8.4, 150 mM NaCl, 2.5 mM CaCl₂) at 277 K, and precipitate was removed by centrifugation at 20,000×g for 20 min at 277 K. To the soluble fraction, 1 unit of thrombin protease (Novagen) was added per milligram of fusion protein, and the His tag was removed by digestion for 4 h at 298 K (thrombin cleavage results in a Rab9a-(1-177) protein with an N terminal GSHMAS extension). The thrombin cleavage reaction was diluted (1:3, v/v) with 20 mM 4-morpholineethanesulfonic acid (MES), pH 6.5, and applied to Q-Sepharose (Amersham Biosciences), which had been previously equilibrated with Q buffer A (20 mM MES, pH 6.5, 50 mM NaCl). Native Rab9-(1-177) was eluted from Q-Sepharose with a 50-750 mM NaCl linear gradient in MES, pH 6.5; fractions containing native Rab9-(1-177) were identified by denaturing gel electrophoresis and pooled. The pooled Q fractions were then further purified by gel filtration on Sephacryl S-200 (Amersham Biosciences) in MES, pH 6.5, 150 mM NaCl; fractions containing Rab9-(1-177) were pooled and concentrated by ultrafiltration.

Crystallization and Data Collection—The stock protein solution used for crystallization contained 20 mM MES buffer, pH 6.5, and 150 mM sodium chloride with a protein concentration of 10 mg/ml. Crystals were grown at 277 K by the hanging-drop vapor diffusion method with 100 mM sodium acetate buffer, pH 5.0, 5% (v/v) polyethylene glycol 4000 as crystallization solution. Crystals formed in space group P₁ with a=38.40 Å, b=45.62 Å, c=51.22 Å, α=99.8°, β=107.2°, and γ=101.8° and contained two monomers in the unit cell. X-ray diffraction data to 1.25-Å resolution were collected at beamline 22-ID in the facilities of the South East Regional Collaborative Access Team at the Advanced Photon Source, Argonne National Laboratory. The statistics for data collection and processing are summarized in Table 1.

Structure Determination and Refinement—The orientation and position of the Rab9 dimer in the P₁ unit cell were determined using the molecular replacement protocols in Crystallography & NMR Software (Brunger, A. T. et al. (1998) Acta Crystallogr. Sect. D Biol. Crystallogr. 54, 905-921) starting from the structure of Rab11a (PDB code 1OIV (Pasqualato, S. et al. (2004) J. Biol. Chem. 279, 11480-11488)) as the search model. The composite omit map was calculated to guide electronic density fitting of the model. Energy-restrained crystallographic refinement was carried out with maximum likelihood algorithms implemented in Crystallography & NMR Software (Brunger, A. T. et al. (1998) Acta Crystallogr. Sect. D Biol. Crystallogr. 54, 905-921). Refinement proceeded through several cycles in combination with manual checking by the program 0 (Jones, T. A. et al. (1991) Acta Crystallogr. Sect. A 47, 110-119). The addition of two GDPs and 473 water molecules and refinement up to 1.25 Å resulted in R and Rfree values of 0.213 and 0.232, respectively. Further refinement was continued with SHELX-97 (Sheldrick, G. M., and Schneider, T. R. (1997) Methods Enzymol. 227, 319-343) by subjecting the structure to cycles of isotropic conjugate gradient least squares refinement; then tightly restrained anisotropic displacement parameters were introduced and refined. The final refinement cycle resulted in R/Rfree values of 0.139/0.196. The final model contains residues 2-34 and 39-175 of monomer A, residues 5-34, 39-110, and 115-175 of monomer B, and 2 GDP molecules plus 508 water molecules. The phasing and refinement statistics are summarized in Table 1.

Protein Fold Analysis—Secondary structure elements were defined by the hydrogen-bonding patterns in combination with visual inspection. The Dali algorithm of comparing protein domain structures by alignment of distance matrices was used to search for structural homologues of Rab9 and also used for structure-based sequence alignment (Holm, L., and Sander, C. (1993) J. Mol. Biol. 233, 123-138; Holm, L., and Sander, C. (1998) Proteins 33, 88-96). Ribbon diagrams were prepared by the program MOLSCRIPT (Kraulis, P. J. (1991) J. Appl. Crystallogr. 24, 946-950).

Results and Discussion

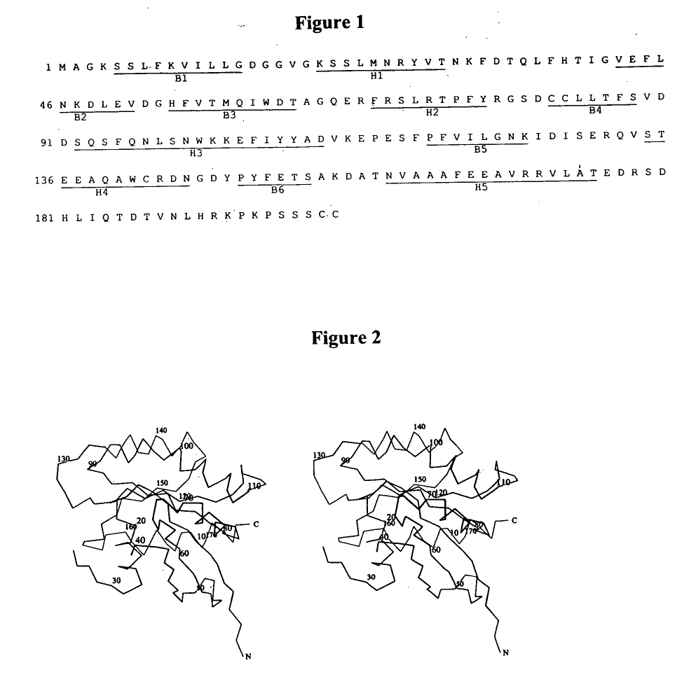

Structure Determination—The human Rab9 variant used for crystal structure determination included residues 1-177, lacking its last 24 residues (FIG. 1). Known as the C-terminal hypervariable region, the amino acid sequence of this region in Rab9 is poorly conserved with respect to other Rab proteins. Therefore, the C-terminal 24 residues were excluded from these cloning and crystallographic studies. This truncated form of the protein will be referred to below as Rab9. Rab9 bound to GDP was crystallized, and its structure was determined by molecular replacement (Table 1). The structure was refined against 1.25-Å resolution data, making it one of the highest resolution structures in the Rab protein family. Both N and C termini (residues 1 and 176-177 of monomer A and residues 1-4 and 176-177 of monomer B) and some loop regions (residues 34-38 of both monomers and residues 111-114 of monomer B) were disordered and could not be seen in the experimental electron density map. The final refined model, which includes residues 2-34 and 39-175 of monomer A, residues 5-34, 39-110, and 115-175 of monomer B, 2 GDP molecules, and 508 ordered water molecules, has a working R value of 0.139 and a free R value of 0.196. The stereochemistry is excellent with root mean square (r.m.s.) deviations for bond lengths and angle distances of 0.013 Å and 0.032 Å, respectively (Table 1). The Ramachandran plot statistics showed that 93.2% of the backbone dihedral angles were in the most favored regions, 6.8% in the additional allowed regions, and none of the non-glycine residues were in the disallowed regions. The two crystallographically unique Rab9 molecules in the crystal unit cell have almost identical structures with the r.m.s. deviation between the 161 equivalent Cα atoms of 0.40 Å. Monomer A will be used in the present description of Rab9 structure.

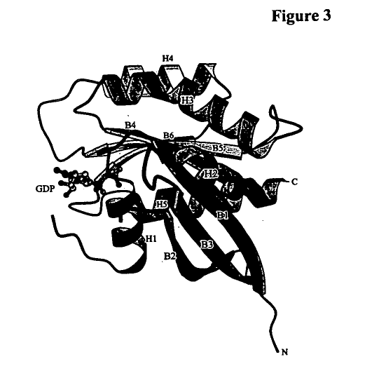

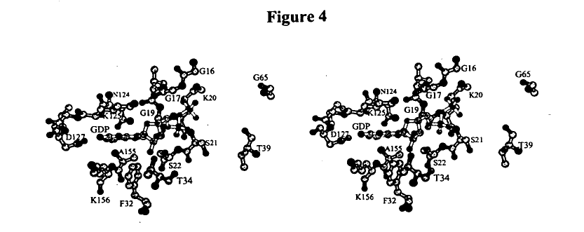

Overall Structure of Rab9 in the Rab9-GDP Complex—Like other members of the Rab GTPase family, Rab9 adopts a classical nucleotide binding fold consisting of a six-stranded β-sheet surrounded by five α-helices (FIGS. 2 and 3). The five α-helices (H1-H5) and six β-strands (B1-B6) connect with a B1-H1-B2-B3-H2-B4-H3-B5-H4-B6-H5 topology containing 30.5% (54/177) α-helix, 28.8% (51/177) β-sheet, 37.3% (66/177) turn/loop, and 3.4% (6/177) other (FIGS. 1 and 3). The six β-strands arrange in the order of B2-B3-B1-B4-B5-B6, forming a mostly parallel β-sheet except that B2 is antiparallel to the rest in strand direction.

Active Site Structure—The crystal structure reported here contains a tightly bound GDP molecule in the active site (FIGS. 3 and 4). Rab9 residues making hydrogen bonds with GDP include Gly-17, Gly-19, Lys-20, Ser-21, Ser-22, Thr-34, Asn-124, Lys-125, Asp-127, and Ala-155 (Table 2, FIG. 4, and as shown by the color red in FIG. 5). These residues together with Gly-16, Val-18, Phe-32, Ile-126, and Lys-156 form a tight binding pocket accommodating the GDP molecule. There are more than a dozen hydrogen bonds formed between Rab9 and GDP, indicating a strong binding affinity of Rab9 for GDP. This is supported by the observation that the GDP molecule has been kept inside the active site throughout Rab9 purification, that is it comes naturally bound to Rab9 from the cell culture. Residues Thr-39 and Gly-65 are highly conserved in the Rab GTPase family and are predicted to interact with the γ-phosphate of GTP in the active state of Rab9.

Structure Comparison—The overall structure of Rab9 is very similar to the prototype Ras protein p21Ras (Scheidig, A. J. et al. (1999) Structure Fold Des. 7, 1311-1324) and several Rab proteins (Table 3). Among those, Rab9 has the highest sequence identity with Ypt7p (54% over 153 equivalent positions), followed by Rab 11a (43% over 161 equivalent positions). The structural similarity Z-scores (Holm, L., and Sander, C. (1993) J. Mol. Biol. 233, 123-138; Holm, L., and Sander, C. (1998) Proteins 33, 88-96) range from 26.6 to 23.2 with r.m.s. deviations of equivalent positions in the range of 1.4-2.0 Å. Structure-based sequence alignment reveals that the active site of Rab9 consists of residues highly conserved in the Rab GTPase family (FIG. 5), implying a common catalytic mechanism. However, Rab9 contains seven hypervariable regions that are significantly different in conformation from other Rab proteins (FIGS. 5 and 6). Some of those regions coincide with putative effector-binding sites and conformational switch I and switch II regions identified by earlier crystallographic studies of other Rab proteins. Regions II and IV correspond to the switch I and switch II, respectively, whereas regions I, V, and VII correspond to the three effector-binding sites/complementary determining regions. Region I of the C-terminally truncated human Rab9 protein of the present invention corresponds to amino acids 1 to 7 of SEQ ID NO:1 (MAGKSSL). Region II of the Rab9 protein corresponds to amino acids 33 to 43 of SEQ ID NO:1 (DTQLFHTIGVE). Region III of the Rab9 protein corresponds to amino acids 50 to 54 of SEQ ID NO:1 (EVDGH). Region IV of the Rab9 protein corresponds to amino acids 63 to 79 of SEQ ID NO:1 (TAGQERFRSLRTPFYRG). Region V of the Rab9 protein corresponds to amino acids 108 to 117 of SEQ ID NO:1 (YADVKEPESF). Region VI of the Rab9 protein corresponds to amino acids 128 to 130 of SEQ ID NO:1 (ISE). Region VII of the Rab9 protein corresponds to amino acids 168 to 177 of SEQ ID NO:1 (RVLATEDRSD). These seven hypervariable regions in Rab9 structure may serve as sites for antiviral drug binding and provide an excellent target for structure-based drug design and development.

Atomic Coordinates and Structure Factors—The atomic coordinates and structure factors (code 1WMS) have been deposited in the Protein Data Bank (PDB), Research Collaboratory for Structural Bioinformatics, Rutgers University, New Brunswick, N.J. (http://www.rcsb.org/). Portions of the information filed therein have been reproduced here in Table 4. All abbreviations, terms, and formats of the listed values follow the PDB Format Guide (Version 2.3, 1998).

### Example 2

### Crystal Structure of Rab9-GppNHp Complex

For this experiment, the crystal structure of a C-terminally truncated human Rab9 (residues 1-177) in complex with the GTP analog Guanosine 5′-(β,γ-imido) triphosphate (GppNHp) was determined.

Methods

Complex Preparation—After the truncated Rab9 (1-177) was expressed in BL21(DE3) (Chen et al. (2004) J Biol. Chem., 279: 40204-40208), and purified by His-affinity chromatography (HiTrap Chelating HP, Amersham Biosciences), the protein was cleaved from His-tags by thrombin protease (Novagen). The protein was then further purified with anion-exchange affinity chromatography (HiTrap Q HP, Amersham Biosciences) at pH6.5. The complex of truncated Rab9 (1-177) and GppNHp trisodium salt (Sigma-Aldrich) was prepared by adding 3× molar excess of GppNHp, 10 units alkaline phosphatase (Sigma-Aldrich) per milligram of truncated Rab9 (1-177), 5 mM MgCl₂, 1 mM DTT, 100 mM NaCl in 20 mM MES pH6.5, and incubated overnight (˜12 hours) at room temperature. The complex solution of truncated Rab9 (1-177) and GppNHp was further purified with preparatory gel filtration chromatography (Sephacryl S-200, Amersham Biosciences).

Crystallization and Data Collection—The complex solution used for crystallization contained 20 mM MES buffer, pH 6.5 and 150 mM sodium chloride with a protein concentration of 15 mg/ml. Crystals were grown at 277 K by the sitting-drop vapor diffusion method with 100 mM sodium acetate buffer, pH 5.0, 5-8% (v/v) polyethylene glycol (PEG) 4000 as crystallization solution. Crystals formed in space group P2₁2₁2, with a=56.24 Å, b=76.60 Å, c=174.35 Å and contained four monomers in the asymmetric unit. X-ray diffraction data to 1.73 Å resolution were collected at beamline 22-ID in the facilities of the South East Regional Collaborative Access Team (SER-CAT) at the Advanced Photon Source, Argonne National Laboratory, USA. The statistics for data collection and processing are summarized in Table 5.

Structure Determination and Refinement—The orientation and position of the four Rab9 monomers in the asymmetric unit were determined using the molecular replacement protocols in the program CNS (Brunger, A. T. et al. (1998) Acta Crystallogr. Sect. D Biol. Crystallogr. 54, 905-921) starting from the dimer structure of Rab9-GDP complex (PDB code 1WMS) as the search model. The composite omit map was calculated to guide electronic density fitting of the model. Energy-restrained crystallographic refinement was carried out with maximum likelihood algorithms implemented in CNS. Refinement proceeded through several cycles in combination with manual checking by the program 0 (Jones, T. A. et al. (1991) Acta Crystallogr. Sect. A 47, 110-119). The final refinement cycle resulted in R/R_{free} values of 0.194/0.223. The final model contains residues 6-175 of monomer A′, residues 4-110, 115-175 of monomer B′, residues 6-174 of monomer C′, residues 6-110,115-175 of monomer D′, four GppNHp molecules, four Magnesium ions plus 495 water molecules. The phasing and refinement statistics are summarized in Table 5.

Results and Discussion

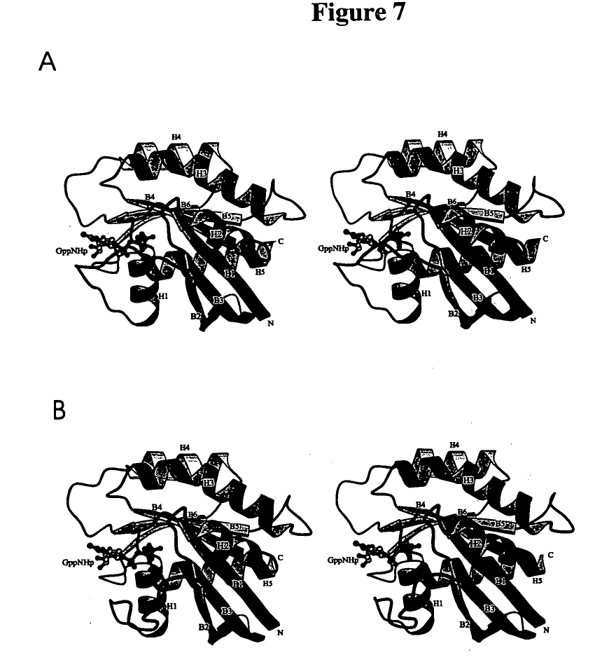

Structure Determination—Rab9 bound to GppNHp was crystallized, and its structure was determined by molecular replacement and refined against 1.73 Å resolution data (Table 5). The refined structure has excellent stereochemistry with r.m.s. deviations for bond lengths and angles of 0.005 Å and 1.20, respectively. The Ramachandran plot statistics shows that 91.4% of the backbone dihedral angles are in the most favored regions, 8.6% in the additional allowed regions and none of the non-glycine residues are in the disallowed regions. There are four crystallographically unique Rab9 molecules in the crystal asymmetric unit. They can be divided into two groups with two monomers each. Monomers A′ and D′ (Group I) have almost identical structures with the r.m.s. deviation between the 166 equivalent Cα atoms of 0.25 Å, while Monomers B′ and C′ (Group II) are very similar with the r.m.s. deviation between the 165 equivalent Cα atoms of 0.52 Å. The r.m.s. deviations between any inter-group pair of monomers are much higher in the range of 1.89 to 1.94 Å. For simplicity, Monomers A′ and C′ will be used in the present description of Rab9-GppNHp complex structures (FIGS. 7a and 7B; Monomers D′ and B′ not shown).

Overall Structure of Rab9 in the Rab9-GppNHp Complex—Rab9 in the complex adopts a classical nucleotide binding fold consisting of a six-stranded α-sheet surrounded by five α-helices (FIG. 7). The 5 α-helices (H1-H5) and 6 β-strands (B1-B6) connect with a B1-H1-B2-B3-H2-B4-H3-B5-H4-B6-H5 topology. The 6 β-strands arrange in the order of B2-B3-B1-B4-B5-B6, forming a mostly parallel α-sheet except that B2 is antiparallel to the rest in strand direction. Monomers A′ and C′ have similar conformations throughout most of the peptide chain except the Switch I region (the loop between α-helix H1 and β-strand B2). In monomer A′, the Switch I region is close to the GppNHp binding site and will be referred to here as “the closed conformation” (FIG. 7A). In monomer C′, the Switch I region is far away from the GppNHp binding site and will be referred to here as “the open conformation” (FIG. 7B). Ribbon diagrams were prepared by the program MOLSCRIPT (Kraulis, P. J. (1991) J. Appl. Crystallogr. 24, 946-950).

Active Site Structure—The crystal structure reported here contains a tightly bound GppNHp molecule in the active site of each monomer (FIG. 7). In monomer A′ (FIG. 7A), Rab9 residues making hydrogen bonds with GppNHp include G17, G19, K20, S21, S22, D33, T34, H38, T39, G65, N124, K125, D127 and A155. There are approximately 20 hydrogen bonds formed between Rab9 and GppNHp, indicating a strong binding affinity of Rab9 for GppNHp. Residues K20, T39 and G65 are highly conserved in the Rab GTPase family, and together with residue H38 interact with the γ-phosphate of GppNHp. In monomer C′ (FIG. 7B), Rab9 residues making hydrogen bonds with GppNHp include G17, G19, K20, S21, S22, T29, K31, G65, N124, K125, D127 and A155. There are about 18 hydrogen bonds formed between Rab9 and GppNHp. Residues K20 and G65 interact with the γ-phosphate of GppNHp. Because the Switch I region (residues 33-40) in monomer C′ is in the open conformation far away from the active site, residues D33, T34, H38 and T39 do not interact with GppNHp in contrast with that of monomer A′. There is one magnesium ion inside the active site of each monomer. In monomer A′, the Mg²⁺ has octahedral coordination, having hydrogen bonds with O2B, O2G of GppNHp, OG of Rab9 S21, OG1 of Rab9 T39 and two water molecules; in monomer C′, the Mg²⁺ has similar coordination except missing the interaction with T39.

Structure Comparison With the Rab9-GDP Complex—The overall structures of Rab9-GppNHp complex are similar to that of Rab9-GDP complex with r.m.s. deviation of equivalent Cα atoms in the range of 1.28 Å for monomer A′ to 1.32 Å for monomer C′ (FIG. 8). However, significant differences exist in the Switch I and Switch II regions. The Switch I region is disordered in the Rab9-GDP complex but well ordered in the Rab9-GppNHp complex. The Switch II region is closer to the nucleotide binding pocket in the Rab9-GppNHp complex than that in the Rab9-GDP complex.

Atomic Coordinates and Structure Factors—Atomic coordinates and structure factors for the Rab9-GppNHp complex have been reproduced here in Table 6. All abbreviations, terms, and formats of the listed values follow the PDB Format Guide (Version 2.3, 1998).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

TABLE 1
Summary of data collection, phasing and refinement

| | |
|---|---|
| Data and Phasing Statistics | |
| Space group | P1 |
| Unit cell: a, b, c (Å), α, β, γ (°) | 38.40, 45.62, 51.22, 99.7, 107.2, 101.8 |
| Wavelength (Å) | 1.00 |
| Resolution (Å) | 1.25 |
| Reflections (total/unique) | 507,012/73,993 |
| Completeness (%)^{a} | 84.7 (39.9) |
| I/σ^{a} | 25.5 (2.3) |
| R(I)_{merge} (%)^{a} | 6.3 (39.2) |
| Molecular replacement model | Rab11a dimer |
| Refinement Statistics | |
| Resolution range (Å) | 10-1.25 |
| Number of reflections | 73,767 |
| Number of atoms | protein/GDP/water, 2662/56/508 |
| R factors (%) | R_{work}/R_{free}, 13.9/19.6 |
| R.m.s. deviation of bonds | length/angle distance, 0.013 Å/0.032 Å |

^{a}Values in parentheses are for the highest resolution shell.

TABLE 2
Hydrogen bonds between Rab9 and GDP

| | | Hydrogen Bond |
|---|---|---|
| Rab9 Atoms | GDP Atoms | Distances (Å) |
| Monomer A | | |
| Gly17 N | O1B | 2.83 |
| Gly19 N | O3A | 3.13 |
| Lys20 N | O2B | 2.85 |
| Lys20 NZ | O2B | 2.81 |
| Ser21 N | O3B | 2.91 |
| Ser22 N | O1A | 2.84 |
| Ser22 OG | O1A | 2.68 |
| Thr34 OG1 | O3* | 3.02 |
| Thr34 OG1 | O2* | 3.05 |
| Lys125 NZ | O4* | 3.04 |
| Asp127 OD1 | N1 | 2.82 |
| Asp127 OD2 | N2 | 2.83 |
| Ala155 N | O6 | 2.86 |
| Monomer B | | |
| Gly17 N | O1B | 2.89 |
| Gly19 N | O3A | 3.13 |
| Gly19 N | O2B | 3.02 |
| Lys20 N | O2B | 2.88 |
| Lys20 NZ | O2B | 2.75 |
| Ser21 N | O3B | 2.96 |
| Ser22 N | O1A | 2.93 |
| Ser22 OG | O1A | 2.70 |
| Thr34 O | O2* | 2.50 |
| Asn124 | N7 | 3.17 |
| Lys125 NZ | O4* | 2.99 |
| Asp127 OD1 | N1 | 2.72 |
| Asp127 OD2 | N2 | 2.83 |
| Ala155 N | O6 | 2.82 |

TABLE 3
Alignment statistics of Rab9 with structurally similar proteins^{a}

| | PDB | | | | | | |
|---|---|---|---|---|---|---|---|
| Protein Name | code | Z^{b} | RMSD^{c} | LALI^{d} | LSEQ2^{e} | % IDE^{f} | Source |
| GDP-bound Rab9 | 1WMS | 35.3 | 0.0 | 168 | 168 | 100 | Present Data |
| Gppnhp-bound | 1KY2 | 26.6 | 2.0 | 167 | 180 | 53 | Constantinescu et |
| Ypt7p | | | | | | | al. (2002) |
| | | | | | | | Structure (Lond.) |
| | | | | | | | 10, 569-579 |
| Gppsp-bound | 1OIW | 25.4 | 1.4 | 158 | 166 | 41 | Pasqualato et al. |
| Rab11a | | | | | | | (2004) J. Biol. |
| | | | | | | | Chem. 279, |
| | | | | | | | 11480-11488 |
| Gppnhp-bound | 1CTQ | 25.2 | 1.6 | 161 | 166 | 31 | Scheidig et al. |
| p21Ras | | | | | | | (1999) Structure |
| | | | | | | | Fold Des. 7, |
| | | | | | | | 1311-1324 |
| GDP-bound | 1OIV | 25.1 | 1.6 | 161 | 168 | 43 | Pasqualato et al. |
| Rab11a | | | | | | | (2004) J. Biol. |
| | | | | | | | Chem. 279, |
| | | | | | | | 11480-11488 |
| GDP-bound Ypt7p | 1KY3 | 24.9 | 1.5 | 153 | 162 | 54 | Constantinescu et |
| | | | | | | | al. (2002) |
| | | | | | | | Structure (Lond.) |
| | | | | | | | 10, 569-579 |
| Gppnhp-bound | 1G17 | 24.7 | 1.6 | 159 | 168 | 38 | Stroupe & Brunger |
| Sec4 | | | | | | | (2000) J. Mol. |
| | | | | | | | Biol. 304, 585-598 |
| Gppnhp-bound | 1N6H | 24.2 | 1.8 | 160 | 167 | 38 | Zhu et al. (2003) J. |
| Rab5a | | | | | | | Biol. Chem. 278, |
| | | | | | | | 2452-2460 |
| Gppnhp-bound | 1HUQ | 24.0 | 1.8 | 159 | 164 | 39 | Merithew et al. |
| Rab5c | | | | | | | (2001) J. Biol. |
| | | | | | | | Chem. 276, |
| | | | | | | | 13982-13988 |
| GDP-bound Sec4 | 1G16 | 23.3 | 1.5 | 150 | 156 | 35 | Stroupe et al. |
| | | | | | | | (2000) J. Mol. |
| | | | | | | | Biol. 304, 585-598 |
| Gppnhp-bound | 1EK0 | 23.2 | 1.8 | 159 | 168 | 40 | Esters et al. (2000) |
| Ypt51 | | | | | | | J. Mol. Biol. 298, |
| | | | | | | | 111-121 |

^{a}Produced by Dali algorithm (Holm, L., and Sander, C. (1993) J. Mol. Biol. 233, 123-138; Holm, L., and Sander, C. (1998) Proteins 33, 88-96).
^{b}Z-score, strength of structural similarity in standard deviations above expected.
^{c}Positional root mean square deviation of superimposed Cα atoms in Å.
^{d}Total number of equivalent residues.
^{e}Length of the entire chain of the equivalent structure.
^{f}Percentage of sequence identity over equivalent positions.

TABLE 4
Atomic Coordinate and Structure Factor Data for the Rab9-GDP Complex

| | | | | | | | | | | | | |
|---|---|---|---|---|---|---|---|---|---|---|---|---|
| ATOM | 1 | N | ALA | A | 2 | 27.804 | −6.394 | 13.944 | 1.00 | 56.13 | | N |
| ANISOU | 1 | N | ALA | A | 2 | 11088 | 3027 | 7213 | 3198 | −21 | 439 | N |
| ATOM | 2 | CA | ALA | A | 2 | 27.496 | −5.012 | 13.588 | 1.00 | 38.78 | | C |
| ANISOU | 2 | CA | ALA | A | 2 | 7211 | 3377 | 4146 | 3026 | 1893 | 904 | C |
| ATOM | 3 | C | ALA | A | 2 | 26.851 | −4.296 | 14.772 | 1.00 | 39.20 | | C |
| ANISOU | 3 | C | ALA | A | 2 | 6755 | 4195 | 3946 | 2056 | 1787 | 273 | C |
| ATOM | 4 | O | ALA | A | 2 | 26.258 | −4.945 | 15.639 | 1.00 | 40.45 | | O |
| ANISOU | 4 | O | ALA | A | 2 | 6384 | 3902 | 5084 | 2529 | 2418 | 557 | O |
| ATOM | 5 | CB | ALA | A | 2 | 26.630 | −4.978 | 12.331 | 1.00 | 54.17 | | C |
| ANISOU | 5 | CB | ALA | A | 2 | 14036 | 2463 | 4083 | 195 | −483 | 1065 | C |
| ATOM | 6 | N | GLY | A | 3 | 26.962 | −2.974 | 14.815 | 1.00 | 35.54 | | N |
| ANISOU | 6 | N | GLY | A | 3 | 4454 | 4474 | 4577 | 971 | 2764 | −617 | N |
| ATOM | 7 | CA | GLY | A | 3 | 26.365 | −2.147 | 15.850 | 1.00 | 40.05 | | C |
| ANISOU | 7 | CA | GLY | A | 3 | 4479 | 5433 | 5304 | 677 | 2830 | −1582 | C |
| ATOM | 8 | C | GLY | A | 3 | 24.862 | −2.193 | 15.899 | 1.00 | 37.33 | | C |
| ANISOU | 8 | C | GLY | A | 3 | 4417 | 4216 | 5551 | 1078 | 2699 | −1094 | C |
| ATOM | 9 | O | GLY | A | 3 | 24.154 | −1.631 | 15.068 | 1.00 | 44.95 | | O |
| ANISOU | 9 | O | GLY | A | 3 | 4381 | 7106 | 5592 | 509 | 3359 | 1250 | O |
| ATOM | 10 | N | LYS | A | 4 | 24.288 | −2.853 | 16.907 | 1.00 | 32.71 | | N |
| ANISOU | 10 | N | LYS | A | 4 | 3050 | 3452 | 5926 | 1292 | 1209 | 23 | N |
| ATOM | 11 | CA | LYS | A | 4 | 22.826 | −2.898 | 16.960 | 1.00 | 35.72 | | C |
| ANISOU | 11 | CA | LYS | A | 4 | 3079 | 3918 | 6574 | 721 | 1265 | −662 | C |
| ATOM | 12 | C | LYS | A | 4 | 22.189 | −1.554 | 17.336 | 1.00 | 30.10 | | C |
| ANISOU | 12 | C | LYS | A | 4 | 1903 | 4040 | 5494 | 170 | 1499 | −1006 | C |
| ATOM | 13 | O | LYS | A | 4 | 22.900 | −0.724 | 17.914 | 1.00 | 29.05 | | O |
| ANISOU | 13 | O | LYS | A | 4 | 4237 | 3404 | 3397 | 100 | −520 | 689 | O |
| ATOM | 14 | CB | LYS | A | 4 | 22.351 | −3.966 | 17.952 | 1.00 | 40.21 | | C |
| ANISOU | 14 | CB | LYS | A | 4 | 3652 | 4265 | 7359 | 489 | 1730 | −392 | C |
| ATOM | 15 | CG | LYS | A | 4 | 22.374 | −5.350 | 17.297 | 1.00 | 49.82 | | C |
| ANISOU | 15 | CG | LYS | A | 4 | 6888 | 3714 | 8329 | 1186 | −374 | −106 | C |
| ATOM | 16 | CD | LYS | A | 4 | 22.938 | −6.349 | 18.282 | 1.00 | 51.44 | | C |
| ANISOU | 16 | CD | LYS | A | 4 | 6864 | 4895 | 7784 | 1570 | −564 | −145 | C |
| ATOM | 17 | CE | LYS | A | 4 | 22.051 | −6.489 | 19.504 | 1.00 | 54.38 | | C |
| ANISOU | 17 | CE | LYS | A | 4 | 6826 | 6402 | 7433 | 1214 | −815 | −178 | C |
| ATOM | 18 | NZ | LYS | A | 4 | 22.720 | −7.285 | 20.586 | 1.00 | 55.74 | | N |
| ANISOU | 18 | NZ | LYS | A | 4 | 4754 | 10361 | 6066 | −1559 | −3190 | −1072 | N |
| ATOM | 19 | N | SER | A | 5 | 20.927 | −1.486 | 16.969 | 1.00 | 32.18 | | N |
| ANISOU | 19 | N | SER | A | 5 | 2331 | 4834 | 5062 | 565 | 926 | −585 | N |
| ATOM | 20 | CA | SER | A | 5 | 19.904 | −0.489 | 16.888 | 1.00 | 30.95 | | C |
| ANISOU | 20 | CA | SER | A | 5 | 2443 | 4774 | 4542 | 687 | 1092 | −1093 | C |
| ATOM | 21 | C | SER | A | 5 | 18.552 | −1.011 | 17.373 | 1.00 | 28.89 | | C |
| ANISOU | 21 | C | SER | A | 5 | 2193 | 3716 | 5069 | 498 | 389 | −1051 | C |
| ATOM | 22 | O | SER | A | 5 | 18.106 | −2.099 | 17.004 | 1.00 | 45.44 | | O |
| ANISOU | 22 | O | SER | A | 5 | 6845 | 3758 | 6663 | −1245 | 1492 | −932 | O |
| ATOM | 23 | CB | SER | A | 5 | 19.705 | −0.018 | 15.437 | 1.00 | 33.99 | | C |
| ANISOU | 23 | CB | SER | A | 5 | 2614 | 5686 | 4616 | 1430 | 962 | −987 | C |
| ATOM | 24 | OG | SER | A | 5 | 18.972 | 1.184 | 15.313 | 1.00 | 39.55 | | O |
| ANISOU | 24 | OG | SER | A | 5 | 3941 | 4850 | 6235 | 998 | 637 | −441 | O |
| ATOM | 25 | N | SER | A | 6 | 17.877 | −0.218 | 18.181 | 1.00 | 25.59 | | N |
| ANISOU | 25 | N | SER | A | 6 | 1696 | 4292 | 3735 | 1081 | 234 | −110 | N |
| ATOM | 26 | CA | SER | A | 6 | 16.533 | −0.626 | 18.587 | 1.00 | 21.58 | | C |
| ANISOU | 26 | CA | SER | A | 6 | 1775 | 3132 | 3293 | 859 | −262 | 515 | C |
| ATOM | 27 | C | SER | A | 6 | 15.442 | 0.069 | 17.795 | 1.00 | 18.88 | | C |
| ANISOU | 27 | C | SER | A | 6 | 1844 | 2405 | 2924 | 306 | −524 | 362 | C |
| ATOM | 28 | O | SER | A | 6 | 15.750 | 1.179 | 17.378 | 1.00 | 21.13 | | O |
| ANISOU | 28 | O | SER | A | 6 | 1256 | 2643 | 4130 | 134 | −556 | 741 | O |
| ATOM | 29 | CB | SER | A | 6 | 16.450 | −0.242 | 20.059 | 1.00 | 28.07 | | C |
| ANISOU | 29 | CB | SER | A | 6 | 2643 | 5331 | 2689 | 590 | −159 | 1252 | C |
| ATOM | 30 | OG | SER | A | 6 | 17.583 | −0.772 | 20.743 | 1.00 | 45.83 | | O |
| ANISOU | 30 | OG | SER | A | 6 | 6888 | 5353 | 5173 | 2259 | −3210 | 986 | O |
| ATOM | 31 | N | LEU | A | 7 | 14.270 | −0.539 | 17.630 | 1.00 | 16.41 | | N |
| ANISOU | 31 | N | LEU | A | 7 | 1628 | 1842 | 2763 | 485 | −24 | 397 | N |
| ATOM | 32 | CA | LEU | A | 7 | 13.174 | −0.004 | 16.809 | 1.00 | 14.17 | | C |
| ANISOU | 32 | CA | LEU | A | 7 | 1560 | 1815 | 2008 | 360 | 17 | −83 | C |
| ATOM | 33 | C | LEU | A | 7 | 12.021 | 0.462 | 17.720 | 1.00 | 13.89 | | C |
| ANISOU | 33 | C | LEU | A | 7 | 1276 | 1895 | 2107 | 188 | −36 | −115 | C |
| ATOM | 34 | O | LEU | A | 7 | 11.471 | −0.323 | 18.500 | 1.00 | 19.42 | | O |
| ANISOU | 34 | O | LEU | A | 7 | 2607 | 1830 | 2942 | −41 | 935 | −186 | O |
| ATOM | 35 | CB | LEU | A | 7 | 12.660 | −0.986 | 15.783 | 1.00 | 15.63 | | C |
| ANISOU | 35 | CB | LEU | A | 7 | 2356 | 1458 | 2124 | −37 | 292 | 16 | C |
| ATOM | 36 | CG | LEU | A | 7 | 11.489 | −0.554 | 14.893 | 1.00 | 18.42 | | C |
| ANISOU | 36 | CG | LEU | A | 7 | 2154 | 2272 | 2573 | −595 | −208 | −465 | C |
| ATOM | 37 | CD1 | LEU | A | 7 | 11.947 | 0.473 | 13.878 | 1.00 | 17.05 | | C |
| ANISOU | 37 | CD1 | LEU | A | 7 | 1524 | 3293 | 1663 | 315 | −205 | −61 | C |
| ATOM | 38 | CD2 | LEU | A | 7 | 10.885 | −1.765 | 14.204 | 1.00 | 28.77 | | C |
| ANISOU | 38 | CD2 | LEU | A | 7 | 4011 | 2917 | 4004 | −526 | −592 | −1712 | C |
| ATOM | 39 | N | PHE | A | 8 | 11.667 | 1.719 | 17.663 | 1.00 | 11.57 | | N |
| ANISOU | 39 | N | PHE | A | 8 | 874 | 1868 | 1654 | 80 | −261 | −231 | N |
| ATOM | 40 | CA | PHE | A | 8 | 10.604 | 2.295 | 18.480 | 1.00 | 12.79 | | C |
| ANISOU | 40 | CA | PHE | A | 8 | 1005 | 2084 | 1771 | 296 | −157 | −74 | C |
| ATOM | 41 | C | PHE | A | 8 | 9.460 | 2.681 | 17.540 | 1.00 | 11.67 | | C |
| ANISOU | 41 | C | PHE | A | 8 | 850 | 2174 | 1410 | 220 | 89 | 16 | C |
| ATOM | 42 | O | PHE | A | 8 | 9.691 | 3.392 | 16.573 | 1.00 | 15.58 | | O |
| ANISOU | 42 | O | PHE | A | 8 | 1053 | 3144 | 1724 | −394 | −175 | 488 | O |
| ATOM | 43 | CB | PHE | A | 8 | 11.081 | 3.534 | 19.220 | 1.00 | 15.95 | | C |
| ANISOU | 43 | CB | PHE | A | 8 | 1563 | 2320 | 2178 | 859 | −627 | −608 | C |
| ATOM | 44 | CG | PHE | A | 8 | 12.170 | 3.301 | 20.241 | 1.00 | 20.83 | | C |
| ANISOU | 44 | CG | PHE | A | 8 | 1611 | 4142 | 2161 | 971 | −713 | −1067 | C |
| ATOM | 45 | CD1 | PHE | A | 8 | 13.433 | 2.964 | 19.898 | 1.00 | 24.18 | | C |
| ANISOU | 45 | CD1 | PHE | A | 8 | 1181 | 5378 | 2628 | 467 | −646 | −411 | C |
| ATOM | 46 | CD2 | PHE | A | 8 | 11.862 | 3.445 | 21.565 | 1.00 | 24.42 | | C |
| ANISOU | 46 | CD2 | PHE | A | 8 | 2082 | 5092 | 2104 | 532 | −630 | −770 | C |
| ATOM | 47 | CE1 | PHE | A | 8 | 14.404 | 2.745 | 20.851 | 1.00 | 23.77 | | C |
| ANISOU | 47 | CE1 | PHE | A | 8 | 1414 | 5173 | 2444 | 436 | −805 | −681 | C |
| ATOM | 48 | CE2 | PHE | A | 8 | 12.796 | 3.203 | 22.539 | 1.00 | 26.27 | | C |
| ANISOU | 48 | CE2 | PHE | A | 8 | 2088 | 5768 | 2125 | 560 | −600 | −458 | C |
| ATOM | 49 | CZ | PHE | A | 8 | 14.073 | 2.840 | 22.178 | 1.00 | 24.11 | | C |
| ANISOU | 49 | CZ | PHE | A | 8 | 1670 | 5017 | 2474 | −175 | −419 | −158 | C |
| ATOM | 50 | N | LYS | A | 9 | 8.246 | 2.255 | 17.857 | 1.00 | 10.25 | | N |
| ANISOU | 50 | N | LYS | A | 9 | 1031 | 1363 | 1501 | −97 | −189 | −77 | N |
| ATOM | 51 | CA | LYS | A | 9 | 7.055 | 2.578 | 17.091 | 1.00 | 9.45 | | C |
| ANISOU | 51 | CA | LYS | A | 9 | 828 | 1435 | 1329 | 34 | 49 | −189 | C |
| ATOM | 52 | C | LYS | A | 9 | 6.319 | 3.755 | 17.747 | 1.00 | 9.37 | | C |
| ANISOU | 52 | C | LYS | A | 9 | 879 | 1498 | 1184 | −149 | 185 | −354 | C |
| ATOM | 53 | O | LYS | A | 9 | 5.992 | 3.662 | 18.933 | 1.00 | 10.76 | | O |
| ANISOU | 53 | O | LYS | A | 9 | 958 | 1961 | 1168 | −254 | 104 | −265 | O |
| ATOM | 54 | CB | LYS | A | 9 | 6.173 | 1.367 | 16.953 | 1.00 | 9.98 | | C |
| ANISOU | 54 | CB | LYS | A | 9 | 835 | 1408 | 1548 | 68 | −2 | −263 | C |
| ATOM | 55 | CG | LYS | A | 9 | 4.881 | 1.623 | 16.219 | 1.00 | 9.11 | | C |
| ANISOU | 55 | CG | LYS | A | 9 | 1054 | 1278 | 1130 | −86 | −71 | −202 | C |
| ATOM | 56 | CD | LYS | A | 9 | 4.037 | 0.375 | 16.024 | 1.00 | 11.64 | | C |
| ANISOU | 56 | CD | LYS | A | 9 | 1034 | 1396 | 1990 | −160 | 24 | −537 | C |
| ATOM | 57 | CE | LYS | A | 9 | 2.706 | 0.666 | 15.394 | 1.00 | 11.46 | | C |
| ANISOU | 57 | CE | LYS | A | 9 | 913 | 1945 | 1497 | −67 | 239 | −672 | C |
| ATOM | 58 | NZ | LYS | A | 9 | 1.887 | −0.588 | 15.249 | 1.00 | 15.27 | | N |
| ANISOU | 58 | NZ | LYS | A | 9 | 983 | 1952 | 2865 | 70 | −489 | −866 | N |
| ATOM | 59 | N | VAL | A | 10 | 6.137 | 4.837 | 17.001 | 1.00 | 8.57 | | N |
| ANISOU | 59 | N | VAL | A | 10 | 505 | 1490 | 1261 | −24 | 34 | −386 | N |
| ATOM | 60 | CA | VAL | A | 10 | 5.420 | 6.026 | 17.507 | 1.00 | 10.54 | | C |
| ANISOU | 60 | CA | VAL | A | 10 | 823 | 1963 | 1219 | 403 | −135 | −403 | C |
| ATOM | 61 | C | VAL | A | 10 | 4.216 | 6.259 | 16.620 | 1.00 | 10.07 | | C |
| ANISOU | 61 | C | VAL | A | 10 | 733 | 1892 | 1201 | 255 | −198 | −740 | C |
| ATOM | 62 | O | VAL | A | 10 | 4.345 | 6.111 | 15.399 | 1.00 | 14.14 | | O |
| ANISOU | 62 | O | VAL | A | 10 | 985 | 3215 | 1174 | 580 | −110 | −556 | O |
| ATOM | 63 | CB | VAL | A | 10 | 6.386 | 7.238 | 17.537 | 1.00 | 15.18 | | C |
| ANISOU | 63 | CB | VAL | A | 10 | 1283 | 1994 | 2490 | 257 | −1000 | −1688 | C |
| ATOM | 64 | CG1 | VAL | A | 10 | 5.717 | 8.534 | 17.903 | 1.00 | 20.94 | | C |
| ANISOU | 64 | CG1 | VAL | A | 10 | 3054 | 1803 | 3097 | 998 | −661 | −418 | C |
| ATOM | 65 | CG2 | VAL | A | 10 | 7.541 | 6.829 | 18.486 | 1.00 | 16.78 | | C |
| ANISOU | 65 | CG2 | VAL | A | 10 | 1808 | 1429 | 3141 | 9 | −1239 | −570 | C |
| ATOM | 66 | N | ILE | A | 11 | 3.102 | 6.597 | 17.221 | 1.00 | 9.97 | | N |
| ANISOU | 66 | N | ILE | A | 11 | 858 | 1700 | 1229 | 461 | −129 | −455 | N |
| ATOM | 67 | CA | ILE | A | 11 | 1.914 | 6.870 | 16.443 | 1.00 | 10.55 | | C |
| ANISOU | 67 | CA | ILE | A | 11 | 897 | 1563 | 1548 | 294 | −389 | −575 | C |
| ATOM | 68 | C | ILE | A | 11 | 1.455 | 8.302 | 16.657 | 1.00 | 8.69 | | C |
| ANISOU | 68 | C | ILE | A | 11 | 722 | 1446 | 1134 | 194 | −37 | −326 | C |
| ATOM | 69 | O | ILE | A | 11 | 1.523 | 8.795 | 17.787 | 1.00 | 11.27 | | O |
| ANISOU | 69 | O | ILE | A | 11 | 1315 | 1721 | 1244 | 289 | −326 | −564 | O |
| ATOM | 70 | CB | ILE | A | 11 | 0.775 | 5.908 | 16.799 | 1.00 | 14.23 | | C |
| ANISOU | 70 | CB | ILE | A | 11 | 1312 | 1462 | 2635 | 3 | −568 | −389 | C |
| ATOM | 71 | CG1 | ILE | A | 11 | −0.468 | 6.044 | 15.954 | 1.00 | 18.05 | | C |
| ANISOU | 71 | CG1 | ILE | A | 11 | 1429 | 1617 | 3812 | −349 | −1054 | 98 | C |
| ATOM | 72 | CG2 | ILE | A | 11 | 0.394 | 6.008 | 18.276 | 1.00 | 16.93 | | C |
| ANISOU | 72 | CG2 | ILE | A | 11 | 1261 | 2227 | 2943 | −35 | 182 | 50 | C |
| ATOM | 73 | CD1 | ILE | A | 11 | −1.354 | 4.842 | 16.100 | 1.00 | 19.20 | | C |
| ANISOU | 73 | CD1 | ILE | A | 11 | 1289 | 1881 | 4127 | −411 | −600 | 105 | C |
| ATOM | 74 | N | LEU | A | 12 | 1.008 | 8.946 | 15.601 | 1.00 | 9.54 | | N |
| ANISOU | 74 | N | LEU | A | 12 | 962 | 1576 | 1085 | 182 | −132 | −455 | N |
| ATOM | 75 | CA | LEU | A | 12 | 0.459 | 10.274 | 15.581 | 1.00 | 9.28 | | C |
| ANISOU | 75 | CA | LEU | A | 12 | 1098 | 1522 | 907 | 169 | −60 | −270 | C |
| ATOM | 76 | C | LEU | A | 12 | −1.035 | 10.152 | 15.483 | 1.00 | 8.73 | | C |
| ANISOU | 76 | C | LEU | A | 12 | 1080 | 1313 | 925 | 219 | −269 | −211 | C |
| ATOM | 77 | O | LEU | A | 12 | −1.523 | 9.544 | 14.562 | 1.00 | 11.69 | | O |
| ANISOU | 77 | O | LEU | A | 12 | 1469 | 1730 | 1244 | 432 | −522 | −635 | O |
| ATOM | 78 | CB | LEU | A | 12 | 0.850 | 11.137 | 14.386 | 1.00 | 14.39 | | C |
| ANISOU | 78 | CB | LEU | A | 12 | 1629 | 2230 | 1609 | −120 | 30 | 337 | C |
| ATOM | 79 | CG | LEU | A | 12 | 2.260 | 11.713 | 14.437 | 1.00 | 15.89 | | C |
| ANISOU | 79 | CG | LEU | A | 12 | 1950 | 1897 | 2190 | −438 | 279 | −73 | C |
| ATOM | 80 | CD1 | LEU | A | 12 | 2.592 | 12.143 | 13.057 | 1.00 | 19.79 | | C |
| ANISOU | 80 | CD1 | LEU | A | 12 | 1843 | 3231 | 2446 | −1007 | −72 | 616 | C |
| ATOM | 81 | CD2 | LEU | A | 12 | 2.298 | 12.931 | 15.286 | 1.00 | 22.43 | | C |
| ANISOU | 81 | CD2 | LEU | A | 12 | 3360 | 1998 | 3163 | 480 | −1709 | −514 | C |
| ATOM | 82 | N | LEU | A | 13 | −1.735 | 10.790 | 16.412 | 1.00 | 8.65 | | N |
| ANISOU | 82 | N | LEU | A | 13 | 1056 | 1307 | 923 | 196 | −167 | −171 | N |
| ATOM | 83 | CA | LEU | A | 13 | −3.203 | 10.841 | 16.392 | 1.00 | 8.83 | | C |
| ANISOU | 83 | CA | LEU | A | 13 | 959 | 1221 | 1177 | 90 | −228 | −60 | C |
| ATOM | 84 | C | LEU | A | 13 | −3.659 | 12.278 | 16.532 | 1.00 | 9.16 | | C |
| ANISOU | 84 | C | LEU | A | 13 | 994 | 1259 | 1227 | 84 | −385 | −61 | C |
| ATOM | 85 | O | LEU | A | 13 | −2.970 | 13.070 | 17.154 | 1.00 | 11.83 | | O |
| ANISOU | 85 | O | LEU | A | 13 | 1505 | 1367 | 1622 | 264 | −906 | −412 | O |
| ATOM | 86 | CB | LEU | A | 13 | −3.741 | 10.054 | 17.543 | 1.00 | 10.96 | | C |
| ANISOU | 86 | CB | LEU | A | 13 | 1221 | 1390 | 1553 | −331 | −271 | 98 | C |
| ATOM | 87 | CG | LEU | A | 13 | −3.491 | 8.550 | 17.560 | 1.00 | 12.83 | | C |
| ANISOU | 87 | CG | LEU | A | 13 | 1891 | 1251 | 1732 | −577 | 8 | −101 | C |
| ATOM | 88 | CD1 | LEU | A | 13 | −4.217 | 7.987 | 18.775 | 1.00 | 14.91 | | C |
| ANISOU | 88 | CD1 | LEU | A | 13 | 2089 | 1677 | 1900 | 247 | 165 | 574 | C |
| ATOM | 89 | CD2 | LEU | A | 13 | −3.920 | 7.838 | 16.301 | 1.00 | 17.51 | | C |
| ANISOU | 89 | CD2 | LEU | A | 13 | 2719 | 2009 | 1925 | −1012 | −48 | −428 | C |
| ATOM | 90 | N | GLY | A | 14 | −4.803 | 12.612 | 15.966 | 1.00 | 7.94 | | N |
| ANISOU | 90 | N | GLY | A | 14 | 810 | 1125 | 1083 | 39 | −230 | −186 | N |
| ATOM | 91 | CA | GLY | A | 14 | −5.355 | 13.941 | 16.094 | 1.00 | 8.61 | | C |
| ANISOU | 91 | CA | GLY | A | 14 | 799 | 1171 | 1299 | 22 | −285 | −187 | C |
| ATOM | 92 | C | GLY | A | 14 | −6.315 | 14.224 | 14.959 | 1.00 | 8.90 | | C |
| ANISOU | 92 | C | GLY | A | 14 | 783 | 1341 | 1257 | 82 | −307 | −302 | C |
| ATOM | 93 | O | GLY | A | 14 | −6.312 | 13.485 | 13.969 | 1.00 | 9.87 | | O |
| ANISOU | 93 | O | GLY | A | 14 | 871 | 1549 | 1331 | 89 | −253 | −436 | O |
| ATOM | 94 | N | ASP | A | 15 | −7.096 | 15.267 | 15.077 | 1.00 | 9.77 | | N |
| ANISOU | 94 | N | ASP | A | 15 | 850 | 1670 | 1192 | 262 | −414 | −456 | N |
| ATOM | 95 | CA | ASP | A | 15 | −8.110 | 15.569 | 14.110 | 1.00 | 9.53 | | C |
| ANISOU | 95 | CA | ASP | A | 15 | 889 | 1490 | 1241 | −120 | −447 | 23 | C |
| ATOM | 96 | C | ASP | A | 15 | −7.508 | 15.758 | 12.715 | 1.00 | 9.85 | | C |
| ANISOU | 96 | C | ASP | A | 15 | 787 | 1642 | 1313 | −105 | −439 | 54 | C |
| ATOM | 97 | O | ASP | A | 15 | −6.370 | 16.163 | 12.511 | 1.00 | 9.35 | | O |
| ANISOU | 97 | O | ASP | A | 15 | 781 | 1523 | 1250 | 10 | −355 | −206 | O |
| ATOM | 98 | CB | ASP | A | 15 | −8.867 | 16.846 | 14.456 | 1.00 | 10.02 | | C |
| ANISOU | 98 | CB | ASP | A | 15 | 916 | 1552 | 1340 | −48 | −570 | −39 | C |
| ATOM | 99 | CG | ASP | A | 15 | −9.858 | 16.705 | 15.595 | 1.00 | 11.17 | | C |
| ANISOU | 99 | CG | ASP | A | 15 | 600 | 1897 | 1747 | −145 | −459 | −317 | C |
| ATOM | 100 | OD1 | ASP | A | 15 | −9.939 | 15.576 | 16.152 | 1.00 | 12.38 | | O |
| ANISOU | 100 | OD1 | ASP | A | 15 | 1050 | 2210 | 1444 | 182 | −200 | 40 | O |
| ATOM | 101 | OD2 | ASP | A | 15 | −10.505 | 17.746 | 15.866 | 1.00 | 14.02 | | O |
| ANISOU | 101 | OD2 | ASP | A | 15 | 1174 | 1963 | 2189 | 15 | −348 | −508 | O |
| ATOM | 102 | N | GLY | A | 16 | −8.344 | 15.490 | 11.723 | 1.00 | 10.63 | | N |
| ANISOU | 102 | N | GLY | A | 16 | 915 | 1817 | 1306 | −116 | −475 | −30 | N |
| ATOM | 103 | CA | GLY | A | 16 | −7.951 | 15.812 | 10.356 | 1.00 | 11.06 | | C |
| ANISOU | 103 | CA | GLY | A | 16 | 1097 | 1769 | 1335 | −222 | −505 | −14 | C |
| ATOM | 104 | C | GLY | A | 16 | −7.555 | 17.282 | 10.214 | 1.00 | 9.58 | | C |
| ANISOU | 104 | C | GLY | A | 16 | 734 | 1717 | 1190 | −84 | −463 | −142 | C |
| ATOM | 105 | O | GLY | A | 16 | −8.222 | 18.169 | 10.737 | 1.00 | 11.02 | | O |
| ANISOU | 105 | O | GLY | A | 16 | 772 | 1782 | 1632 | −54 | −103 | −68 | O |
| ATOM | 106 | N | GLY | A | 17 | −6.447 | 17.497 | 9.499 | 1.00 | 9.36 | | N |
| ANISOU | 106 | N | GLY | A | 17 | 779 | 1592 | 1183 | 46 | −351 | −126 | N |
| ATOM | 107 | CA | GLY | A | 17 | −6.026 | 18.835 | 9.184 | 1.00 | 9.99 | | C |
| ANISOU | 107 | CA | GLY | A | 17 | 914 | 1688 | 1195 | −29 | −409 | 28 | C |
| ATOM | 108 | C | GLY | A | 17 | −5.088 | 19.473 | 10.168 | 1.00 | 9.37 | | C |
| ANISOU | 108 | C | GLY | A | 17 | 1117 | 1439 | 1005 | 0 | −353 | −21 | C |
| ATOM | 109 | O | GLY | A | 17 | −4.658 | 20.616 | 9.912 | 1.00 | 10.80 | | O |
| ANISOU | 109 | O | GLY | A | 17 | 1103 | 1583 | 1417 | −84 | −343 | 89 | O |
| ATOM | 110 | N | VAL | A | 18 | −4.792 | 18.840 | 11.272 | 1.00 | 8.56 | | N |
| ANISOU | 110 | N | VAL | A | 18 | 809 | 1403 | 1040 | 121 | −312 | −86 | N |
| ATOM | 111 | CA | VAL | A | 18 | −3.943 | 19.490 | 12.271 | 1.00 | 8.55 | | C |
| ANISOU | 111 | CA | VAL | A | 18 | 773 | 1601 | 873 | 145 | −122 | −392 | C |
| ATOM | 112 | C | VAL | A | 18 | −2.474 | 19.497 | 11.876 | 1.00 | 7.79 | | C |
| ANISOU | 112 | C | VAL | A | 18 | 793 | 1081 | 1086 | 53 | −163 | −123 | C |
| ATOM | 113 | O | VAL | A | 18 | −1.728 | 20.342 | 12.435 | 1.00 | 9.05 | | O |
| ANISOU | 113 | O | VAL | A | 18 | 900 | 1252 | 1286 | 7 | −301 | −54 | O |
| ATOM | 114 | CB | VAL | A | 18 | −4.088 | 18.868 | 13.668 | 1.00 | 8.66 | | C |
| ANISOU | 114 | CB | VAL | A | 18 | 702 | 1676 | 912 | 204 | −216 | −309 | C |
| ATOM | 115 | CG1 | VAL | A | 18 | −5.510 | 19.000 | 14.200 | 1.00 | 10.26 | | C |
| ANISOU | 115 | CG1 | VAL | A | 18 | 899 | 1880 | 1120 | 204 | 51 | −291 | C |
| ATOM | 116 | CG2 | VAL | A | 18 | −3.640 | 17.420 | 13.763 | 1.00 | 9.06 | | C |
| ANISOU | 116 | CG2 | VAL | A | 18 | 745 | 1465 | 1231 | −118 | −207 | −345 | C |
| ATOM | 117 | N | GLY | A | 19 | −2.071 | 18.652 | 10.952 | 1.00 | 8.39 | | N |
| ANISOU | 117 | N | GLY | A | 19 | 861 | 1430 | 897 | 113 | 2 | −49 | N |
| ATOM | 118 | CA | GLY | A | 19 | −0.696 | 18.691 | 10.467 | 1.00 | 8.32 | | C |
| ANISOU | 118 | CA | GLY | A | 19 | 781 | 1315 | 1067 | 65 | −118 | 24 | C |
| ATOM | 119 | C | GLY | A | 19 | 0.064 | 17.419 | 10.730 | 1.00 | 8.36 | | C |
| ANISOU | 119 | C | GLY | A | 19 | 918 | 1228 | 1032 | 143 | −2 | −57 | C |
| ATOM | 120 | O | GLY | A | 19 | 1.321 | 17.451 | 10.649 | 1.00 | 8.47 | | O |
| ANISOU | 120 | O | GLY | A | 19 | 949 | 1183 | 1086 | 95 | −241 | 29 | O |
| ATOM | 121 | N | LYS | A | 20 | −0.563 | 16.299 | 11.005 | 1.00 | 8.81 | | N |
| ANISOU | 121 | N | LYS | A | 20 | 919 | 1365 | 1063 | 35 | −262 | 8 | N |
| ATOM | 122 | CA | LYS | A | 20 | 0.136 | 15.040 | 11.297 | 1.00 | 7.69 | | C |
| ANISOU | 122 | CA | LYS | A | 20 | 776 | 1241 | 903 | −75 | −114 | −29 | C |
| ATOM | 123 | C | LYS | A | 20 | 1.039 | 14.596 | 10.166 | 1.00 | 8.44 | | C |
| ANISOU | 123 | C | LYS | A | 20 | 696 | 1449 | 1063 | −140 | −102 | −66 | C |
| ATOM | 124 | O | LYS | A | 20 | 2.252 | 14.288 | 10.348 | 1.00 | 9.17 | | O |
| ANISOU | 124 | O | LYS | A | 20 | 800 | 1523 | 1161 | 31 | −197 | −217 | O |
| ATOM | 125 | CB | LYS | A | 20 | −0.861 | 13.921 | 11.644 | 1.00 | 8.28 | | C |
| ANISOU | 125 | CB | LYS | A | 20 | 611 | 1422 | 1114 | −44 | −174 | 66 | C |
| ATOM | 126 | CG | LYS | A | 20 | −1.712 | 14.280 | 12.873 | 1.00 | 9.48 | | C |
| ANISOU | 126 | CG | LYS | A | 20 | 723 | 1622 | 1259 | 55 | 11 | 126 | C |
| ATOM | 127 | CD | LYS | A | 20 | −2.738 | 13.218 | 13.168 | 1.00 | 9.46 | | C |
| ANISOU | 127 | CD | LYS | A | 20 | 731 | 1565 | 1300 | 64 | 0 | 50 | C |
| ATOM | 128 | CE | LYS | A | 20 | −3.707 | 12.852 | 12.069 | 1.00 | 11.74 | | C |
| ANISOU | 128 | CE | LYS | A | 20 | 1158 | 1449 | 1852 | 46 | −503 | 88 | C |
| ATOM | 129 | NZ | LYS | A | 20 | −4.556 | 13.992 | 11.647 | 1.00 | 8.68 | | N |
| ANISOU | 129 | NZ | LYS | A | 20 | 896 | 1261 | 1140 | −63 | −111 | −40 | N |
| ATOM | 130 | N | SER | A | 21 | 0.497 | 14.514 | 8.970 | 1.00 | 9.43 | | N |
| ANISOU | 130 | N | SER | A | 21 | 833 | 1745 | 1007 | −39 | −137 | −390 | N |
| ATOM | 131 | CA | SER | A | 21 | 1.314 | 14.103 | 7.853 | 1.00 | 9.81 | | C |
| ANISOU | 131 | CA | SER | A | 21 | 807 | 1578 | 1340 | 198 | −29 | −473 | C |
| ATOM | 132 | C | SER | A | 21 | 2.459 | 15.057 | 7.615 | 1.00 | 9.75 | | C |
| ANISOU | 132 | C | SER | A | 21 | 889 | 1688 | 1127 | 202 | −28 | −201 | C |
| ATOM | 133 | O | SER | A | 21 | 3.582 | 14.638 | 7.321 | 1.00 | 9.97 | | O |
| ANISOU | 133 | O | SER | A | 21 | 821 | 1718 | 1249 | 202 | −13 | −106 | O |
| ATOM | 134 | CB | SER | A | 21 | 0.419 | 14.055 | 6.590 | 1.00 | 15.07 | | C |
| ANISOU | 134 | CB | SER | A | 21 | 2095 | 2469 | 1162 | −473 | −387 | −817 | C |
| ATOM | 135 | OG | SER | A | 21 | 1.240 | 13.725 | 5.498 | 1.00 | 24.11 | | O |
| ANISOU | 135 | OG | SER | A | 21 | 2137 | 5401 | 1622 | 32 | −224 | −1119 | O |
| ATOM | 136 | N | SER | A | 22 | 2.165 | 16.345 | 7.685 | 1.00 | 9.48 | | N |
| ANISOU | 136 | N | SER | A | 22 | 817 | 1687 | 1097 | 110 | 22 | −237 | N |
| ATOM | 137 | CA | SER | A | 22 | 3.182 | 17.362 | 7.460 | 1.00 | 9.58 | | C |
| ANISOU | 137 | CA | SER | A | 22 | 880 | 1786 | 975 | 13 | −175 | 12 | C |
| ATOM | 138 | C | SER | A | 22 | 4.284 | 17.267 | 8.519 | 1.00 | 9.85 | | C |
| ANISOU | 138 | C | SER | A | 22 | 733 | 1898 | 1111 | 89 | −104 | 295 | C |
| ATOM | 139 | O | SER | A | 22 | 5.454 | 17.443 | 8.182 | 1.00 | 9.41 | | O |
| ANISOU | 139 | O | SER | A | 22 | 693 | 1630 | 1251 | 116 | 23 | 46 | O |
| ATOM | 140 | CB | SER | A | 22 | 2.563 | 18.748 | 7.483 | 1.00 | 10.71 | | C |
| ANISOU | 140 | CB | SER | A | 22 | 866 | 1807 | 1395 | 32 | −271 | 274 | C |
| ATOM | 141 | OG | SER | A | 22 | 1.632 | 18.905 | 6.406 | 1.00 | 11.16 | | O |
| ANISOU | 141 | OG | SER | A | 22 | 1055 | 1854 | 1333 | −15 | −327 | 246 | O |
| ATOM | 142 | N | LEU | A | 23 | 3.957 | 16.998 | 9.753 | 1.00 | 8.71 | | N |
| ANISOU | 142 | N | LEU | A | 23 | 724 | 1494 | 1092 | −41 | −99 | 91 | N |
| ATOM | 143 | CA | LEU | A | 23 | 4.952 | 16.874 | 10.812 | 1.00 | 9.17 | | C |
| ANISOU | 143 | CA | LEU | A | 23 | 891 | 1652 | 942 | 127 | −87 | −108 | C |
| ATOM | 144 | C | LEU | A | 23 | 5.878 | 15.707 | 10.575 | 1.00 | 9.69 | | C |
| ANISOU | 144 | C | LEU | A | 23 | 790 | 1927 | 964 | 246 | −224 | −363 | C |
| ATOM | 145 | O | LEU | A | 23 | 7.091 | 15.831 | 10.698 | 1.00 | 9.61 | | O |
| ANISOU | 145 | O | LEU | A | 23 | 786 | 1707 | 1158 | 144 | −131 | −27 | O |
| ATOM | 146 | CB | LEU | A | 23 | 4.237 | 16.748 | 12.151 | 1.00 | 8.44 | | C |
| ANISOU | 146 | CB | LEU | A | 23 | 783 | 1364 | 1059 | 87 | 26 | −49 | C |
| ATOM | 147 | CG | LEU | A | 23 | 3.700 | 18.039 | 12.757 | 1.00 | 8.71 | | C |
| ANISOU | 147 | CG | LEU | A | 23 | 877 | 1439 | 994 | 64 | −253 | −304 | C |
| ATOM | 148 | CD1 | LEU | A | 23 | 2.673 | 17.783 | 13.844 | 1.00 | 12.13 | | C |
| ANISOU | 148 | CD1 | LEU | A | 23 | 1047 | 2360 | 1203 | −176 | 42 | −816 | C |
| ATOM | 149 | CD2 | LEU | A | 23 | 4.848 | 18.893 | 13.272 | 1.00 | 10.85 | | C |
| ANISOU | 149 | CD2 | LEU | A | 23 | 909 | 1690 | 1524 | 29 | −322 | −414 | C |
| ATOM | 150 | N | MET | A | 24 | 5.280 | 14.565 | 10.241 | 1.00 | 10.07 | | N |
| ANISOU | 150 | N | MET | A | 24 | 735 | 1835 | 1257 | 251 | −306 | −165 | N |
| ATOM | 151 | CA | MET | A | 24 | 6.140 | 13.428 | 9.964 | 1.00 | 11.28 | | C |
| ANISOU | 151 | CA | MET | A | 24 | 1220 | 1740 | 1327 | 247 | −107 | −126 | C |
| ATOM | 152 | C | MET | A | 24 | 7.044 | 13.640 | 8.755 | 1.00 | 11.61 | | C |
| ANISOU | 152 | C | MET | A | 24 | 940 | 2478 | 993 | 202 | −293 | −569 | C |
| ATOM | 153 | O | MET | A | 24 | 8.209 | 13.285 | 8.795 | 1.00 | 11.21 | | O |
| ANISOU | 153 | O | MET | A | 24 | 994 | 1824 | 1443 | 193 | −308 | −717 | O |
| ATOM | 154 | CB | MET | A | 24 | 5.270 | 12.288 | 9.645 | 1.00 | 18.17 | | C |
| ANISOU | 154 | CB | MET | A | 24 | 1821 | 1979 | 3102 | −63 | 150 | −310 | C |
| ATOM | 155 | CG | MET | A | 24 | 5.635 | 10.895 | 9.266 | 1.00 | 22.26 | | C |
| ANISOU | 155 | CG | MET | A | 24 | 2577 | 2321 | 3560 | −322 | −122 | −1160 | C |
| ATOM | 156 | SD | MET | A | 24 | 4.347 | 9.606 | 9.111 | 1.00 | 23.01 | | S |
| ANISOU | 156 | SD | MET | A | 24 | 2828 | 2424 | 3493 | −538 | −782 | 79 | S |
| ATOM | 157 | CE | MET | A | 24 | 3.233 | 10.479 | 10.079 | 1.00 | 23.18 | | C |
| ANISOU | 157 | CE | MET | A | 24 | 1583 | 804 | 6421 | −373 | −1358 | −280 | C |
| ATOM | 158 | N | ASN | A | 25 | 6.464 | 14.217 | 7.715 | 1.00 | 10.58 | | N |
| ANISOU | 158 | N | ASN | A | 25 | 975 | 1991 | 1056 | 264 | −45 | −411 | N |
| ATOM | 159 | CA | ASN | A | 25 | 7.243 | 14.460 | 6.504 | 1.00 | 11.82 | | C |
| ANISOU | 159 | CA | ASN | A | 25 | 1143 | 2235 | 1115 | 217 | 71 | −528 | C |
| ATOM | 160 | C | ASN | A | 25 | 8.343 | 15.464 | 6.784 | 1.00 | 12.40 | | C |
| ANISOU | 160 | C | ASN | A | 25 | 1024 | 2205 | 1483 | 191 | 19 | −356 | C |
| ATOM | 161 | O | ASN | A | 25 | 9.455 | 15.336 | 6.281 | 1.00 | 13.68 | | O |
| ANISOU | 161 | O | ASN | A | 25 | 1108 | 2308 | 1782 | 295 | 162 | −423 | O |
| ATOM | 162 | CB | ASN | A | 25 | 6.302 | 14.892 | 5.370 | 1.00 | 15.96 | | C |
| ANISOU | 162 | CB | ASN | A | 25 | 1473 | 3812 | 781 | 44 | −31 | −482 | C |
| ATOM | 163 | CG | ASN | A | 25 | 5.652 | 13.633 | 4.797 | 1.00 | 23.72 | | C |
| ANISOU | 163 | CG | ASN | A | 25 | 2029 | 5013 | 1970 | −364 | −237 | −1715 | C |
| ATOM | 164 | OD1 | ASN | A | 25 | 6.367 | 12.775 | 4.280 | 1.00 | 30.79 | | O |
| ANISOU | 164 | OD1 | ASN | A | 25 | 2794 | 4273 | 4632 | 515 | −1361 | −1906 | O |
| ATOM | 165 | ND2 | ASN | A | 25 | 4.352 | 13.490 | 4.860 | 1.00 | 37.82 | | N |
| ANISOU | 165 | ND2 | ASN | A | 25 | 2298 | 7979 | 4095 | −1411 | 440 | −3814 | N |
| ATOM | 166 | N | ARG | A | 26 | 8.034 | 16.512 | 7.559 | 1.00 | 11.74 | | N |
| ANISOU | 166 | N | ARG | A | 26 | 1012 | 2032 | 1417 | 154 | 114 | −226 | N |
| ATOM | 167 | CA | ARG | A | 26 | 9.055 | 17.493 | 7.909 | 1.00 | 11.35 | | C |
| ANISOU | 167 | CA | ARG | A | 26 | 1169 | 1597 | 1546 | 166 | −30 | 127 | C |
| ATOM | 168 | C | ARG | A | 26 | 10.204 | 16.841 | 8.672 | 1.00 | 10.37 | | C |
| ANISOU | 168 | C | ARG | A | 26 | 1060 | 1378 | 1501 | 181 | −3 | −109 | C |
| ATOM | 169 | O | ARG | A | 26 | 11.392 | 17.058 | 8.401 | 1.00 | 12.05 | | O |
| ANISOU | 169 | O | ARG | A | 26 | 1105 | 1588 | 1886 | 142 | 101 | 7 | O |
| ATOM | 170 | CB | ARG | A | 26 | 8.501 | 18.630 | 8.763 | 1.00 | 14.06 | | C |
| ANISOU | 170 | CB | ARG | A | 26 | 1459 | 1914 | 1968 | 655 | −438 | −233 | C |
| ATOM | 171 | CG | ARG | A | 26 | 9.515 | 19.695 | 9.182 | 1.00 | 17.01 | | C |
| ANISOU | 171 | CG | ARG | A | 26 | 2166 | 1758 | 2538 | 591 | −643 | −370 | C |
| ATOM | 172 | CD | ARG | A | 26 | 9.812 | 20.459 | 7.893 | 1.00 | 31.10 | | C |
| ANISOU | 172 | CD | ARG | A | 26 | 4815 | 3815 | 3186 | −1467 | 171 | 104 | C |
| ATOM | 173 | NE | ARG | A | 26 | 10.607 | 21.591 | 7.774 | 1.00 | 31.48 | | N |
| ANISOU | 173 | NE | ARG | A | 26 | 5912 | 4165 | 1886 | −2295 | −248 | −693 | N |
| ATOM | 174 | CZ | ARG | A | 26 | 11.297 | 22.286 | 6.924 | 1.00 | 37.02 | | C |
| ANISOU | 174 | CZ | ARG | A | 26 | 8883 | 3807 | 1375 | −3022 | −1279 | 771 | C |
| ATOM | 175 | NH1 | ARG | A | 26 | 11.343 | 21.866 | 5.661 | 1.00 | 46.97 | | N |
| ANISOU | 175 | NH1 | ARG | A | 26 | 7648 | 7198 | 3001 | −3090 | 1934 | −1794 | N |
| ATOM | 176 | NH2 | ARG | A | 26 | 11.986 | 23.391 | 7.199 | 1.00 | 29.98 | | N |
| ANISOU | 176 | NH2 | ARG | A | 26 | 3164 | 3905 | 4320 | −1099 | −186 | −389 | N |
| ATOM | 177 | N | TYR | A | 27 | 9.870 | 16.019 | 9.667 | 1.00 | 10.01 | | N |
| ANISOU | 177 | N | TYR | A | 27 | 966 | 1405 | 1433 | 57 | −215 | −130 | N |
| ATOM | 178 | CA | TYR | A | 27 | 10.885 | 15.383 | 10.482 | 1.00 | 9.21 | | C |
| ANISOU | 178 | CA | TYR | A | 27 | 817 | 1105 | 1579 | −9 | −212 | −151 | C |
| ATOM | 179 | C | TYR | A | 27 | 11.795 | 14.509 | 9.645 | 1.00 | 11.26 | | C |
| ANISOU | 179 | C | TYR | A | 27 | 968 | 1837 | 1475 | 141 | −336 | −575 | C |
| ATOM | 180 | O | TYR | A | 27 | 13.025 | 14.524 | 9.762 | 1.00 | 11.98 | | O |
| ANISOU | 180 | O | TYR | A | 27 | 881 | 1692 | 1977 | 28 | −180 | −478 | O |
| ATOM | 181 | CB | TYR | A | 27 | 10.242 | 14.599 | 11.637 | 1.00 | 11.25 | | C |
| ANISOU | 181 | CB | TYR | A | 27 | 845 | 1724 | 1707 | −52 | −208 | 132 | C |
| ATOM | 182 | CG | TYR | A | 27 | 11.214 | 13.928 | 12.545 | 1.00 | 10.15 | | C |
| ANISOU | 182 | CG | TYR | A | 27 | 1025 | 1409 | 1422 | 75 | −136 | −95 | C |
| ATOM | 183 | CD1 | TYR | A | 27 | 12.298 | 14.635 | 13.075 | 1.00 | 11.11 | | C |
| ANISOU | 183 | CD1 | TYR | A | 27 | 1194 | 1342 | 1685 | 21 | −386 | −115 | C |
| ATOM | 184 | CD2 | TYR | A | 27 | 11.074 | 12.604 | 12.879 | 1.00 | 9.68 | | C |
| ANISOU | 184 | CD2 | TYR | A | 27 | 930 | 1354 | 1393 | 13 | 14 | −184 | C |
| ATOM | 185 | CE1 | TYR | A | 27 | 13.183 | 13.984 | 13.912 | 1.00 | 11.23 | | C |
| ANISOU | 185 | CE1 | TYR | A | 27 | 1336 | 1584 | 1348 | −97 | −347 | −35 | C |
| ATOM | 186 | CE2 | TYR | A | 27 | 11.974 | 11.983 | 13.719 | 1.00 | 10.67 | | C |
| ANISOU | 186 | CE2 | TYR | A | 27 | 1197 | 1481 | 1375 | −50 | 57 | 134 | C |
| ATOM | 187 | CZ | TYR | A | 27 | 13.028 | 12.671 | 14.238 | 1.00 | 11.48 | | C |
| ANISOU | 187 | CZ | TYR | A | 27 | 1355 | 1523 | 1485 | 135 | −320 | −58 | C |
| ATOM | 188 | OH | TYR | A | 27 | 13.912 | 12.028 | 15.063 | 1.00 | 14.65 | | O |
| ANISOU | 188 | OH | TYR | A | 27 | 1898 | 1639 | 2030 | 521 | −648 | −183 | O |
| ATOM | 189 | N | VAL | A | 28 | 11.200 | 13.681 | 8.787 | 1.00 | 11.23 | | N |
| ANISOU | 189 | N | VAL | A | 28 | 970 | 1556 | 1741 | 144 | −360 | −511 | N |
| ATOM | 190 | CA | VAL | A | 28 | 11.952 | 12.673 | 8.050 | 1.00 | 12.41 | | C |
| ANISOU | 190 | CA | VAL | A | 28 | 1438 | 1322 | 1955 | 333 | −332 | −426 | C |
| ATOM | 191 | C | VAL | A | 28 | 12.644 | 13.249 | 6.840 | 1.00 | 13.14 | | C |
| ANISOU | 191 | C | VAL | A | 28 | 1124 | 1929 | 1939 | 181 | −188 | −675 | C |
| ATOM | 192 | O | VAL | A | 28 | 13.766 | 12.834 | 6.563 | 1.00 | 18.43 | | O |
| ANISOU | 192 | O | VAL | A | 28 | 1353 | 1975 | 3674 | 336 | 338 | −460 | O |
| ATOM | 193 | CB | VAL | A | 28 | 10.993 | 11.530 | 7.659 | 1.00 | 13.01 | | C |
| ANISOU | 193 | CB | VAL | A | 28 | 1640 | 1532 | 1773 | 163 | −426 | −491 | C |
| ATOM | 194 | CG1 | VAL | A | 28 | 11.683 | 10.537 | 6.739 | 1.00 | 15.37 | | C |
| ANISOU | 194 | CG1 | VAL | A | 28 | 1338 | 2112 | 2389 | −237 | −119 | −1086 | C |
| ATOM | 195 | CG2 | VAL | A | 28 | 10.473 | 10.787 | 8.886 | 1.00 | 13.10 | | C |
| ANISOU | 195 | CG2 | VAL | A | 28 | 1629 | 1520 | 1828 | 8 | −483 | −498 | C |
| ATOM | 196 | N | THR | A | 29 | 12.045 | 14.186 | 6.110 | 1.00 | 13.13 | | N |
| ANISOU | 196 | N | THR | A | 29 | 1175 | 2061 | 1752 | 159 | 222 | −383 | N |
| ATOM | 197 | CA | THR | A | 29 | 12.610 | 14.656 | 4.846 | 1.00 | 15.03 | | C |
| ANISOU | 197 | CA | THR | A | 29 | 1640 | 2333 | 1740 | 325 | 632 | −643 | C |
| ATOM | 198 | C | THR | A | 29 | 13.083 | 16.108 | 4.876 | 1.00 | 13.84 | | C |
| ANISOU | 198 | C | THR | A | 29 | 1465 | 2086 | 1707 | 660 | 382 | −18 | C |
| ATOM | 199 | O | THR | A | 29 | 13.711 | 16.543 | 3.888 | 1.00 | 17.65 | | O |
| ANISOU | 199 | O | THR | A | 29 | 1989 | 2705 | 2012 | 526 | 687 | 148 | O |
| ATOM | 200 | CB | THR | A | 29 | 11.635 | 14.574 | 3.657 | 1.00 | 18.12 | | C |
| ANISOU | 200 | CB | THR | A | 29 | 2042 | 3186 | 1657 | −91 | 502 | −236 | C |
| ATOM | 201 | OG1 | THR | A | 29 | 10.586 | 15.548 | 3.794 | 1.00 | 18.05 | | O |
| ANISOU | 201 | OG1 | THR | A | 29 | 2057 | 3100 | 1700 | −69 | 75 | 36 | O |
| ATOM | 202 | CG2 | THR | A | 29 | 10.900 | 13.249 | 3.561 | 1.00 | 25.46 | | C |
| ANISOU | 202 | CG2 | THR | A | 29 | 2421 | 3110 | 4141 | 8 | −995 | −390 | C |
| ATOM | 203 | N | ASN | A | 30 | 12.808 | 16.847 | 5.950 | 1.00 | 14.39 | | N |
| ANISOU | 203 | N | ASN | A | 30 | 1285 | 2218 | 1964 | 307 | 260 | −453 | N |
| ATOM | 204 | CA | ASN | A | 30 | 13.108 | 18.254 | 6.126 | 1.00 | 14.30 | | C |
| ANISOU | 204 | CA | ASN | A | 30 | 979 | 2366 | 2089 | 51 | 300 | −307 | C |
| ATOM | 205 | C | ASN | A | 30 | 12.507 | 19.171 | 5.070 | 1.00 | 14.45 | | C |
| ANISOU | 205 | C | ASN | A | 30 | 1265 | 2246 | 1978 | −75 | 484 | −235 | C |
| ATOM | 206 | O | ASN | A | 30 | 13.037 | 20.227 | 4.706 | 1.00 | 20.06 | | O |
| ANISOU | 206 | O | ASN | A | 30 | 2123 | 2338 | 3162 | −465 | 195 | 82 | O |
| ATOM | 207 | CB | ASN | A | 30 | 14.628 | 18.530 | 6.236 | 1.00 | 23.36 | | C |
| ANISOU | 207 | CB | ASN | A | 30 | 1067 | 3873 | 3934 | −512 | 58 | 523 | C |
| ATOM | 208 | CG | ASN | A | 30 | 14.811 | 19.909 | 6.850 | 1.00 | 37.54 | | C |
| ANISOU | 208 | CG | ASN | A | 30 | 2553 | 4911 | 6798 | −1960 | −108 | −842 | C |
| ATOM | 209 | OD1 | ASN | A | 30 | 14.071 | 20.408 | 7.711 | 1.00 | 35.86 | | O |
| ANISOU | 209 | OD1 | ASN | A | 30 | 2446 | 2980 | 8200 | 32 | −710 | −640 | O |
| ATOM | 210 | ND2 | ASN | A | 30 | 15.856 | 20.593 | 6.395 | 1.00 | 51.25 | | N |
| ANISOU | 210 | ND2 | ASN | A | 30 | 3635 | 5798 | 10040 | −2876 | 284 | 44 | N |
| ATOM | 211 | N | LYS | A | 31 | 11.338 | 18.803 | 4.576 | 1.00 | 15.24 | | N |
| ANISOU | 211 | N | LYS | A | 31 | 1340 | 2287 | 2165 | 81 | 127 | 25 | N |
| ATOM | 212 | CA | LYS | A | 31 | 10.596 | 19.535 | 3.559 | 1.00 | 17.17 | | C |
| ANISOU | 212 | CA | LYS | A | 31 | 1757 | 2837 | 1929 | 664 | 397 | 87 | C |
| ATOM | 213 | C | LYS | A | 31 | 9.160 | 19.761 | 4.036 | 1.00 | 15.43 | | C |
| ANISOU | 213 | C | LYS | A | 31 | 1522 | 2253 | 2088 | 292 | 212 | 342 | C |
| ATOM | 214 | O | LYS | A | 31 | 8.614 | 18.969 | 4.797 | 1.00 | 14.83 | | O |
| ANISOU | 214 | O | LYS | A | 31 | 1498 | 2427 | 1711 | 258 | −118 | 395 | O |
| ATOM | 215 | CB | LYS | A | 31 | 10.501 | 18.798 | 2.213 | 1.00 | 22.20 | | C |
| ANISOU | 215 | CB | LYS | A | 31 | 2564 | 3826 | 2046 | 966 | 50 | −257 | C |
| ATOM | 216 | CG | LYS | A | 31 | 11.868 | 18.540 | 1.602 | 1.00 | 31.22 | | C |
| ANISOU | 216 | CG | LYS | A | 31 | 2898 | 6298 | 2668 | 2097 | −98 | −1728 | C |
| ATOM | 217 | CD | LYS | A | 31 | 11.846 | 17.515 | 0.486 | 1.00 | 39.37 | | C |
| ANISOU | 217 | CD | LYS | A | 31 | 4194 | 6985 | 3781 | 1505 | 254 | −2613 | C |
| ATOM | 218 | CE | LYS | A | 31 | 13.172 | 17.639 | −0.272 | 1.00 | 44.30 | | C |
| ANISOU | 218 | CE | LYS | A | 31 | 4204 | 8175 | 4452 | 3172 | 787 | −2683 | C |
| ATOM | 219 | NZ | LYS | A | 31 | 13.087 | 16.906 | −1.566 | 1.00 | 57.52 | | N |
| ANISOU | 219 | NZ | LYS | A | 31 | 5311 | 11067 | 5475 | 822 | 1608 | −4339 | N |
| ATOM | 220 | N | PHE | A | 32 | 8.561 | 20.843 | 3.564 | 1.00 | 13.91 | | N |
| ANISOU | 220 | N | PHE | A | 32 | 1587 | 1760 | 1939 | 173 | 210 | −46 | N |
| ATOM | 221 | CA | PHE | A | 32 | 7.164 | 21.124 | 3.731 | 1.00 | 12.44 | | C |
| ANISOU | 221 | CA | PHE | A | 32 | 1400 | 1409 | 1919 | −87 | 137 | −57 | C |
| ATOM | 222 | C | PHE | A | 32 | 6.628 | 21.643 | 2.404 | 1.00 | 15.30 | | C |
| ANISOU | 222 | C | PHE | A | 32 | 1538 | 2221 | 2054 | 62 | 137 | 91 | C |
| ATOM | 223 | O | PHE | A | 32 | 7.200 | 22.568 | 1.826 | 1.00 | 16.84 | | O |
| ANISOU | 223 | O | PHE | A | 32 | 2088 | 2759 | 1553 | −308 | 25 | 205 | O |
| ATOM | 224 | CB | PHE | A | 32 | 6.957 | 22.171 | 4.832 | 1.00 | 14.69 | | C |
| ANISOU | 224 | CB | PHE | A | 32 | 1266 | 1887 | 2426 | −10 | 49 | −531 | C |
| ATOM | 225 | CG | PHE | A | 32 | 5.511 | 22.654 | 4.993 | 1.00 | 12.00 | | C |
| ANISOU | 225 | CG | PHE | A | 32 | 1133 | 1543 | 1882 | −69 | −75 | −60 | C |
| ATOM | 226 | CD1 | PHE | A | 32 | 4.676 | 21.965 | 5.838 | 1.00 | 11.66 | | C |
| ANISOU | 226 | CD1 | PHE | A | 32 | 1411 | 1512 | 1508 | 216 | −57 | 96 | C |
| ATOM | 227 | CD2 | PHE | A | 32 | 5.012 | 23.759 | 4.309 | 1.00 | 13.17 | | C |
| ANISOU | 227 | CD2 | PHE | A | 32 | 1527 | 1710 | 1766 | −155 | 80 | 218 | C |
| ATOM | 228 | CE1 | PHE | A | 32 | 3.375 | 22.343 | 6.005 | 1.00 | 14.73 | | C |
| ANISOU | 228 | CE1 | PHE | A | 32 | 1355 | 1995 | 2245 | 31 | 271 | 196 | C |
| ATOM | 229 | CE2 | PHE | A | 32 | 3.706 | 24.159 | 4.482 | 1.00 | 14.47 | | C |
| ANISOU | 229 | CE2 | PHE | A | 32 | 1619 | 2004 | 1874 | 228 | −330 | 218 | C |
| ATOM | 230 | CZ | PHE | A | 32 | 2.910 | 23.452 | 5.339 | 1.00 | 14.33 | | C |
| ANISOU | 230 | CZ | PHE | A | 32 | 1153 | 2228 | 2065 | 349 | −48 | −15 | C |
| ATOM | 231 | N | ASP | A | 33 | 5.501 | 21.117 | 1.966 | 1.00 | 15.24 | | N |
| ANISOU | 231 | N | ASP | A | 33 | 2005 | 1880 | 1905 | 51 | −244 | −81 | N |
| ATOM | 232 | CA | ASP | A | 33 | 4.869 | 21.662 | 0.761 | 1.00 | 17.99 | | C |
| ANISOU | 232 | CA | ASP | A | 33 | 2151 | 2522 | 2161 | 671 | −256 | 77 | C |
| ATOM | 233 | C | ASP | A | 33 | 3.380 | 21.421 | 0.836 | 1.00 | 18.87 | | C |
| ANISOU | 233 | C | ASP | A | 33 | 2167 | 2161 | 2841 | 454 | −593 | −181 | C |
| ATOM | 234 | O | ASP | A | 33 | 2.937 | 20.275 | 0.822 | 1.00 | 26.24 | | O |
| ANISOU | 234 | O | ASP | A | 33 | 2889 | 2211 | 4869 | 276 | −581 | −272 | O |
| ATOM | 235 | CB | ASP | A | 33 | 5.498 | 20.980 | −0.451 | 1.00 | 26.52 | | C |
| ANISOU | 235 | CB | ASP | A | 33 | 2832 | 5246 | 1998 | 803 | −447 | −793 | C |
| ATOM | 236 | CG | ASP | A | 33 | 5.125 | 21.568 | −1.789 | 1.00 | 31.66 | | C |
| ANISOU | 236 | CG | ASP | A | 33 | 3368 | 6413 | 2248 | 724 | 314 | 276 | C |
| ATOM | 237 | OD1 | ASP | A | 33 | 4.010 | 22.129 | −1.921 | 1.00 | 38.19 | | O |
| ANISOU | 237 | OD1 | ASP | A | 33 | 4174 | 7736 | 2600 | 1685 | −258 | 766 | O |
| ATOM | 238 | OD2 | ASP | A | 33 | 5.943 | 21.481 | −2.737 | 1.00 | 47.92 | | O |
| ANISOU | 238 | OD2 | ASP | A | 33 | 5838 | 9545 | 2824 | 1975 | 1611 | 202 | O |
| ATOM | 239 | N | THR | A | 34 | 2.549 | 22.457 | 0.870 | 1.00 | 18.59 | | N |
| ANISOU | 239 | N | THR | A | 34 | 1990 | 2212 | 2860 | 341 | −459 | −293 | N |
| ATOM | 240 | CA | THR | A | 34 | 1.099 | 22.223 | 0.832 | 1.00 | 19.50 | | C |
| ANISOU | 240 | CA | THR | A | 34 | 1955 | 2959 | 2494 | 425 | −252 | 569 | C |
| ATOM | 241 | C | THR | A | 34 | 0.679 | 21.786 | −0.577 | 1.00 | 30.35 | | C |
| ANISOU | 241 | C | THR | A | 34 | 3932 | 3672 | 3928 | 1265 | −2784 | −18 | C |
| ATOM | 242 | O | THR | A | 34 | −0.476 | 21.350 | −0.730 | 1.00 | 44.55 | | O |
| ANISOU | 242 | O | THR | A | 34 | 4440 | 5242 | 7246 | 572 | −3269 | −1201 | O |
| ATOM | 243 | CB | THR | A | 34 | 0.372 | 23.484 | 1.321 | 1.00 | 22.34 | | C |
| ANISOU | 243 | CB | THR | A | 34 | 2223 | 2987 | 3276 | 861 | −44 | 1098 | C |
| ATOM | 244 | OG1 | THR | A | 34 | −0.954 | 23.206 | 1.798 | 1.00 | 28.63 | | O |
| ANISOU | 244 | OG1 | THR | A | 34 | 2413 | 5305 | 3161 | 673 | 465 | 67 | O |
| ATOM | 245 | CG2 | THR | A | 34 | 0.223 | 24.452 | 0.160 | 1.00 | 26.70 | | C |
| ANISOU | 245 | CG2 | THR | A | 34 | 3665 | 3330 | 3151 | 641 | −1069 | 1116 | C |
| ATOM | 246 | N | THR | A | 39 | −1.092 | 9.001 | 2.051 | 1.00 | 57.22 | | N |
| ANISOU | 246 | N | THR | A | 39 | 3877 | 7434 | 10431 | 195 | 1545 | −841 | N |
| ATOM | 247 | CA | THR | A | 39 | −1.780 | 10.244 | 2.399 | 1.00 | 49.36 | | C |
| ANISOU | 247 | CA | THR | A | 39 | 4637 | 6193 | 7924 | −268 | 208 | −315 | C |
| ATOM | 248 | C | THR | A | 39 | −3.293 | 10.085 | 2.486 | 1.00 | 35.77 | | C |
| ANISOU | 248 | C | THR | A | 39 | 4322 | 3518 | 5752 | 950 | −633 | 1767 | C |
| ATOM | 249 | O | THR | A | 39 | −3.925 | 10.871 | 3.201 | 1.00 | 43.28 | | O |
| ANISOU | 249 | O | THR | A | 39 | 4938 | 5829 | 5677 | 1684 | −1194 | 531 | O |
| ATOM | 250 | CB | THR | A | 39 | −1.442 | 11.392 | 1.426 | 1.00 | 54.41 | | C |
| ANISOU | 250 | CB | THR | A | 39 | 5304 | 7410 | 7959 | −1682 | 669 | −185 | C |
| ATOM | 251 | OG1 | THR | A | 39 | −2.532 | 11.618 | 0.523 | 1.00 | 50.70 | | O |
| ANISOU | 251 | OG1 | THR | A | 39 | 5221 | 6794 | 7248 | −3231 | 896 | 1078 | O |
| ATOM | 252 | CG2 | THR | A | 39 | −0.221 | 11.059 | 0.572 | 1.00 | 62.72 | | C |
| ANISOU | 252 | CG2 | THR | A | 39 | 5698 | 9168 | 8964 | −1951 | 1235 | −1070 | C |
| ATOM | 253 | N | ILE | A | 40 | −3.924 | 9.125 | 1.840 | 1.00 | 37.19 | | N |
| ANISOU | 253 | N | ILE | A | 40 | 4418 | 4963 | 4749 | 96 | 27 | 1431 | N |
| ATOM | 254 | CA | ILE | A | 40 | −5.352 | 8.846 | 1.965 | 1.00 | 33.99 | | C |
| ANISOU | 254 | CA | ILE | A | 40 | 3921 | 4563 | 4430 | 1116 | −740 | 1561 | C |
| ATOM | 255 | C | ILE | A | 40 | −5.569 | 7.714 | 2.968 | 1.00 | 28.25 | | C |
| ANISOU | 255 | C | ILE | A | 40 | 2852 | 3971 | 3910 | 1168 | −50 | 828 | C |
| ATOM | 256 | O | ILE | A | 40 | −6.591 | 7.649 | 3.660 | 1.00 | 30.60 | | O |
| ANISOU | 256 | O | ILE | A | 40 | 3261 | 4204 | 4161 | 117 | 418 | −958 | O |
| ATOM | 257 | CB | ILE | A | 40 | −5.905 | 8.539 | 0.562 | 1.00 | 41.70 | | C |
| ANISOU | 257 | CB | ILE | A | 40 | 4999 | 6734 | 4112 | 216 | −450 | 1557 | C |
| ATOM | 258 | CG1 | ILE | A | 40 | −6.065 | 9.829 | −0.269 | 1.00 | 40.15 | | C |
| ANISOU | 258 | CG1 | ILE | A | 40 | 4506 | 7650 | 3100 | −282 | 529 | 2160 | C |
| ATOM | 259 | CG2 | ILE | A | 40 | −7.194 | 7.741 | 0.582 | 1.00 | 51.58 | | C |
| ANISOU | 259 | CG2 | ILE | A | 40 | 7798 | 7915 | 3885 | −2442 | −1288 | 1907 | C |
| ATOM | 260 | CD1 | ILE | A | 40 | −5.890 | 9.579 | −1.744 | 1.00 | 45.11 | | C |
| ANISOU | 260 | CD1 | ILE | A | 40 | 6451 | 7587 | 3101 | −3328 | −1038 | 562 | C |
| ATOM | 261 | N | GLY | A | 41 | −4.607 | 6.814 | 3.089 | 1.00 | 24.57 | | N |
| ANISOU | 261 | N | GLY | A | 41 | 2603 | 3771 | 2962 | 766 | −1362 | 327 | N |
| ATOM | 262 | CA | GLY | A | 41 | −4.709 | 5.665 | 3.991 | 1.00 | 19.66 | | C |
| ANISOU | 262 | CA | GLY | A | 41 | 1714 | 3427 | 2328 | 661 | −941 | −206 | C |
| ATOM | 263 | C | GLY | A | 41 | −3.648 | 5.745 | 5.047 | 1.00 | 13.22 | | C |
| ANISOU | 263 | C | GLY | A | 41 | 1618 | 1818 | 1587 | 477 | −397 | −575 | C |
| ATOM | 264 | O | GLY | A | 41 | −3.081 | 6.815 | 5.255 | 1.00 | 20.46 | | O |
| ANISOU | 264 | O | GLY | A | 41 | 3568 | 1709 | 2496 | 45 | −916 | −511 | O |
| ATOM | 265 | N | VAL | A | 42 | −3.384 | 4.632 | 5.663 | 1.00 | 11.97 | | N |
| ANISOU | 265 | N | VAL | A | 42 | 1152 | 1744 | 1653 | 101 | −529 | −512 | N |
| ATOM | 266 | CA | VAL | A | 42 | −2.333 | 4.575 | 6.661 | 1.00 | 10.95 | | C |
| ANISOU | 266 | CA | VAL | A | 42 | 1068 | 1670 | 1423 | −28 | −427 | −546 | C |
| ATOM | 267 | C | VAL | A | 42 | −0.979 | 4.670 | 6.007 | 1.00 | 11.47 | | C |
| ANISOU | 267 | C | VAL | A | 42 | 1065 | 1875 | 1419 | 28 | −368 | −637 | C |
| ATOM | 268 | O | VAL | A | 42 | −0.751 | 4.070 | 4.951 | 1.00 | 13.13 | | O |
| ANISOU | 268 | O | VAL | A | 42 | 1106 | 2264 | 1619 | 115 | −507 | −937 | O |
| ATOM | 269 | CB | VAL | A | 42 | −2.513 | 3.245 | 7.431 | 1.00 | 12.46 | | C |
| ANISOU | 269 | CB | VAL | A | 42 | 1364 | 1896 | 1475 | 203 | −216 | −377 | C |
| ATOM | 270 | CG1 | VAL | A | 42 | −1.363 | 3.130 | 8.427 | 1.00 | 14.89 | | C |
| ANISOU | 270 | CG1 | VAL | A | 42 | 2009 | 1801 | 1848 | 624 | −705 | −522 | C |
| ATOM | 271 | CG2 | VAL | A | 42 | −3.891 | 3.172 | 8.055 | 1.00 | 15.16 | | C |
| ANISOU | 271 | CG2 | VAL | A | 42 | 1692 | 2128 | 1938 | −391 | 204 | −819 | C |
| ATOM | 272 | N | GLU | A | 43 | −0.066 | 5.423 | 6.641 | 1.00 | 10.96 | | N |
| ANISOU | 272 | N | GLU | A | 43 | 852 | 1970 | 1341 | 190 | −319 | −709 | N |
| ATOM | 273 | CA | GLU | A | 43 | 1.286 | 5.560 | 6.170 | 1.00 | 10.35 | | C |
| ANISOU | 273 | CA | GLU | A | 43 | 851 | 1950 | 1131 | 233 | −285 | −811 | C |
| ATOM | 274 | C | GLU | A | 43 | 2.268 | 5.460 | 7.354 | 1.00 | 9.67 | | C |
| ANISOU | 274 | C | GLU | A | 43 | 877 | 1785 | 1010 | 159 | −335 | −834 | C |
| ATOM | 275 | O | GLU | A | 43 | 1.941 | 5.904 | 8.451 | 1.00 | 11.69 | | O |
| ANISOU | 275 | O | GLU | A | 43 | 897 | 2334 | 1212 | 215 | −190 | −1026 | O |
| ATOM | 276 | CB | GLU | A | 43 | 1.553 | 6.874 | 5.426 | 1.00 | 16.68 | | C |
| ANISOU | 276 | CB | GLU | A | 43 | 1822 | 3018 | 1500 | −225 | −470 | 142 | C |
| ATOM | 277 | CG | GLU | A | 43 | 3.018 | 6.983 | 4.904 | 1.00 | 21.43 | | C |
| ANISOU | 277 | CG | GLU | A | 43 | 2026 | 3555 | 2563 | −697 | −138 | 287 | C |
| ATOM | 278 | CD | GLU | A | 43 | 3.294 | 8.322 | 4.222 | 1.00 | 23.98 | | C |
| ANISOU | 278 | CD | GLU | A | 43 | 2146 | 3476 | 3491 | −151 | 251 | 627 | C |
| ATOM | 279 | OE1 | GLU | A | 43 | 2.399 | 9.190 | 4.314 | 1.00 | 34.32 | | O |
| ANISOU | 279 | OE1 | GLU | A | 43 | 3389 | 4008 | 5642 | 805 | −383 | 122 | O |
| ATOM | 280 | OE2 | GLU | A | 43 | 4.371 | 8.536 | 3.604 | 1.00 | 34.24 | | O |
| ANISOU | 280 | OE2 | GLU | A | 43 | 3592 | 4586 | 4831 | −657 | 1523 | 1090 | O |
| ATOM | 281 | N | PHE | A | 44 | 3.399 | 4.859 | 7.118 | 1.00 | 9.61 | | N |
| ANISOU | 281 | N | PHE | A | 44 | 707 | 1841 | 1104 | −24 | −165 | −668 | N |
| ATOM | 282 | CA | PHE | A | 44 | 4.430 | 4.939 | 8.169 | 1.00 | 9.78 | | C |
| ANISOU | 282 | CA | PHE | A | 44 | 661 | 1791 | 1262 | −123 | −220 | −330 | C |
| ATOM | 283 | C | PHE | A | 44 | 5.786 | 5.124 | 7.552 | 1.00 | 8.52 | | C |
| ANISOU | 283 | C | PHE | A | 44 | 642 | 1248 | 1349 | 187 | −43 | −513 | C |
| ATOM | 284 | O | PHE | A | 44 | 6.039 | 4.666 | 6.437 | 1.00 | 11.36 | | O |
| ANISOU | 284 | O | PHE | A | 44 | 906 | 2137 | 1273 | 108 | −165 | −637 | O |
| ATOM | 285 | CB | PHE | A | 44 | 4.411 | 3.740 | 9.085 | 1.00 | 10.53 | | C |
| ANISOU | 285 | CB | PHE | A | 44 | 1007 | 1698 | 1298 | −23 | −64 | −353 | C |
| ATOM | 286 | CG | PHE | A | 44 | 4.438 | 2.355 | 8.484 | 1.00 | 13.71 | | C |
| ANISOU | 286 | CG | PHE | A | 44 | 1875 | 1796 | 1538 | 115 | −185 | −515 | C |
| ATOM | 287 | CD1 | PHE | A | 44 | 3.290 | 1.796 | 7.971 | 1.00 | 13.50 | | C |
| ANISOU | 287 | CD1 | PHE | A | 44 | 2038 | 1779 | 1314 | −424 | 213 | −479 | C |
| ATOM | 288 | CD2 | PHE | A | 44 | 5.588 | 1.636 | 8.496 | 1.00 | 14.51 | | C |
| ANISOU | 288 | CD2 | PHE | A | 44 | 2229 | 2069 | 1213 | 502 | 38 | −548 | C |
| ATOM | 289 | CE1 | PHE | A | 44 | 3.272 | 0.509 | 7.447 | 1.00 | 13.77 | | C |
| ANISOU | 289 | CE1 | PHE | A | 44 | 1633 | 1887 | 1714 | 144 | −164 | −637 | C |
| ATOM | 290 | CE2 | PHE | A | 44 | 5.590 | 0.337 | 7.944 | 1.00 | 13.46 | | C |
| ANISOU | 290 | CE2 | PHE | A | 44 | 1746 | 1645 | 1722 | 133 | −245 | −139 | C |
| ATOM | 291 | CZ | PHE | A | 44 | 4.437 | −0.235 | 7.479 | 1.00 | 13.49 | | C |
| ANISOU | 291 | CZ | PHE | A | 44 | 1367 | 2209 | 1550 | 82 | 51 | −24 | C |
| ATOM | 292 | N | LEU | A | 45 | 6.662 | 5.848 | 8.248 | 1.00 | 10.65 | | N |
| ANISOU | 292 | N | LEU | A | 45 | 633 | 2204 | 1209 | −115 | −47 | −520 | N |
| ATOM | 293 | CA | LEU | A | 45 | 7.982 | 6.292 | 7.834 | 1.00 | 10.94 | | C |
| ANISOU | 293 | CA | LEU | A | 45 | 563 | 2030 | 1563 | −24 | −165 | −263 | C |
| ATOM | 294 | C | LEU | A | 45 | 9.006 | 5.898 | 8.901 | 1.00 | 10.92 | | C |
| ANISOU | 294 | C | LEU | A | 45 | 660 | 2201 | 1288 | 115 | −84 | −445 | C |
| ATOM | 295 | O | LEU | A | 45 | 8.672 | 5.798 | 10.081 | 1.00 | 15.03 | | O |
| ANISOU | 295 | O | LEU | A | 45 | 968 | 3408 | 1336 | 25 | −22 | −268 | O |
| ATOM | 296 | CB | LEU | A | 45 | 8.053 | 7.818 | 7.614 | 1.00 | 13.32 | | C |
| ANISOU | 296 | CB | LEU | A | 45 | 1051 | 2097 | 1914 | −113 | −209 | −158 | C |
| ATOM | 297 | CG | LEU | A | 45 | 7.387 | 8.477 | 6.436 | 1.00 | 19.93 | | C |
| ANISOU | 297 | CG | LEU | A | 45 | 1708 | 2354 | 3508 | 108 | −1074 | 396 | C |
| ATOM | 298 | CD1 | LEU | A | 45 | 7.760 | 9.935 | 6.263 | 1.00 | 29.60 | | C |
| ANISOU | 298 | CD1 | LEU | A | 45 | 4671 | 2346 | 4229 | −258 | −1727 | 651 | C |
| ATOM | 299 | CD2 | LEU | A | 45 | 7.909 | 7.820 | 5.186 | 1.00 | 30.28 | | C |
| ANISOU | 299 | CD2 | LEU | A | 45 | 6325 | 3216 | 1965 | −211 | −1500 | 390 | C |
| ATOM | 300 | N | ASN | A | 46 | 10.243 | 5.677 | 8.529 | 1.00 | 10.79 | | N |
| ANISOU | 300 | N | ASN | A | 46 | 693 | 1928 | 1478 | 128 | −165 | −546 | N |
| ATOM | 301 | CA | ASN | A | 46 | 11.327 | 5.345 | 9.441 | 1.00 | 9.04 | | C |
| ANISOU | 301 | CA | ASN | A | 46 | 551 | 1763 | 1122 | 29 | 46 | −420 | C |
| ATOM | 302 | C | ASN | A | 46 | 12.387 | 6.456 | 9.461 | 1.00 | 10.27 | | C |
| ANISOU | 302 | C | ASN | A | 46 | 589 | 1933 | 1380 | −78 | 137 | −382 | C |
| ATOM | 303 | O | ASN | A | 46 | 12.607 | 7.143 | 8.484 | 1.00 | 11.37 | | O |
| ANISOU | 303 | O | ASN | A | 46 | 978 | 1683 | 1660 | −228 | −154 | −206 | O |
| ATOM | 304 | CB | ASN | A | 46 | 11.996 | 4.043 | 9.084 | 1.00 | 12.72 | | C |
| ANISOU | 304 | CB | ASN | A | 46 | 904 | 1806 | 2123 | 226 | −44 | −420 | C |
| ATOM | 305 | CG | ASN | A | 46 | 11.061 | 2.872 | 9.018 | 1.00 | 12.17 | | C |
| ANISOU | 305 | CG | ASN | A | 46 | 1214 | 1783 | 1628 | 85 | −165 | −351 | C |
| ATOM | 306 | OD1 | ASN | A | 46 | 10.547 | 2.420 | 10.030 | 1.00 | 14.15 | | O |
| ANISOU | 306 | OD1 | ASN | A | 46 | 1351 | 2190 | 1838 | 113 | 150 | −270 | O |
| ATOM | 307 | ND2 | ASN | A | 46 | 10.797 | 2.362 | 7.820 | 1.00 | 15.14 | | N |
| ANISOU | 307 | ND2 | ASN | A | 46 | 1554 | 2374 | 1824 | 139 | −17 | −842 | N |
| ATOM | 308 | N | LYS | A | 47 | 13.018 | 6.627 | 10.631 | 1.00 | 10.80 | | N |
| ANISOU | 308 | N | LYS | A | 47 | 773 | 1735 | 1597 | −156 | −147 | −235 | N |
| ATOM | 309 | CA | LYS | A | 47 | 14.082 | 7.603 | 10.786 | 1.00 | 10.83 | | C |
| ANISOU | 309 | CA | LYS | A | 47 | 826 | 1539 | 1750 | 16 | −311 | −140 | C |
| ATOM | 310 | C | LYS | A | 47 | 15.102 | 7.095 | 11.803 | 1.00 | 9.82 | | C |
| ANISOU | 310 | C | LYS | A | 47 | 621 | 1780 | 1328 | 129 | −61 | −264 | C |
| ATOM | 311 | O | LYS | A | 47 | 14.721 | 6.631 | 12.876 | 1.00 | 11.19 | | O |
| ANISOU | 311 | O | LYS | A | 47 | 846 | 1973 | 1432 | −62 | −55 | −205 | O |
| ATOM | 312 | CB | LYS | A | 47 | 13.502 | 8.957 | 11.237 | 1.00 | 17.54 | | C |
| ANISOU | 312 | CB | LYS | A | 47 | 1571 | 1708 | 3386 | 450 | −1123 | −759 | C |
| ATOM | 313 | CG | LYS | A | 47 | 14.637 | 9.983 | 11.393 | 1.00 | 23.44 | | C |
| ANISOU | 313 | CG | LYS | A | 47 | 2199 | 1888 | 4820 | 178 | −2146 | −936 | C |
| ATOM | 314 | CD | LYS | A | 47 | 14.323 | 11.384 | 11.123 | 1.00 | 18.98 | | C |
| ANISOU | 314 | CD | LYS | A | 47 | 1838 | 2103 | 3273 | −120 | −866 | −97 | C |
| ATOM | 315 | CB | LYS | A | 47 | 15.428 | 12.404 | 11.301 | 1.00 | 17.45 | | C |
| ANISOU | 315 | CB | LYS | A | 47 | 1244 | 2107 | 3278 | 167 | −88 | −809 | C |
| ATOM | 316 | NZ | LYS | A | 47 | 16.209 | 12.534 | 10.030 | 1.00 | 37.42 | | N |
| ANISOU | 316 | NZ | LYS | A | 47 | 5780 | 4965 | 3476 | −3007 | 1179 | −1214 | N |
| ATOM | 317 | N | ASP | A | 48 | 16.375 | 7.175 | 11.424 | 1.00 | 10.75 | | N |
| ANISOU | 317 | N | ASP | A | 48 | 709 | 1950 | 1426 | 97 | −89 | −212 | N |
| ATOM | 318 | CA | ASP | A | 48 | 17.448 | 6.827 | 12.322 | 1.00 | 11.08 | | C |
| ANISOU | 318 | CA | ASP | A | 48 | 696 | 1905 | 1608 | −9 | −235 | −63 | C |
| ATOM | 319 | C | ASP | A | 48 | 17.775 | 7.978 | 13.270 | 1.00 | 10.90 | | C |
| ANISOU | 319 | C | ASP | A | 48 | 738 | 1812 | 1591 | −249 | −159 | 12 | C |
| ATOM | 320 | O | ASP | A | 48 | 17.763 | 9.141 | 12.856 | 1.00 | 12.20 | | O |
| ANISOU | 320 | O | ASP | A | 48 | 1124 | 1834 | 1677 | −47 | −373 | 71 | O |
| ATOM | 321 | CB | ASP | A | 48 | 18.698 | 6.541 | 11.537 | 1.00 | 15.80 | | C |
| ANISOU | 321 | CB | ASP | A | 48 | 634 | 3390 | 1981 | 129 | −270 | −771 | C |
| ATOM | 322 | CG | ASP | A | 48 | 18.742 | 5.136 | 10.976 | 1.00 | 16.70 | | C |
| ANISOU | 322 | CG | ASP | A | 48 | 1168 | 3305 | 1871 | 78 | 173 | −687 | C |
| ATOM | 323 | OD1 | ASP | A | 48 | 17.905 | 4.306 | 11.384 | 1.00 | 18.67 | | O |
| ANISOU | 323 | OD1 | ASP | A | 48 | 1585 | 3184 | 2324 | 141 | 36 | −34 | O |
| ATOM | 324 | OD2 | ASP | A | 48 | 19.619 | 4.929 | 10.137 | 1.00 | 21.59 | | O |
| ANISOU | 324 | OD2 | ASP | A | 48 | 2034 | 3882 | 2287 | 600 | 756 | −544 | O |
| ATOM | 325 | N | LEU | A | 49 | 18.037 | 7.653 | 14.526 | 1.00 | 11.09 | | N |
| ANISOU | 325 | N | LEU | A | 49 | 1070 | 1432 | 1710 | −103 | −348 | −35 | N |
| ATOM | 326 | CA | LEU | A | 49 | 18.550 | 8.692 | 15.426 | 1.00 | 12.04 | | C |
| ANISOU | 326 | CA | LEU | A | 49 | 1031 | 1615 | 1930 | 142 | −470 | −345 | C |
| ATOM | 327 | C | LEU | A | 49 | 19.435 | 7.968 | 16.429 | 1.00 | 10.58 | | C |
| ANISOU | 327 | C | LEU | A | 49 | 1086 | 1179 | 1753 | −54 | −347 | −300 | C |
| ATOM | 328 | O | LEU | A | 49 | 19.801 | 6.810 | 16.263 | 1.00 | 10.72 | | O |
| ANISOU | 328 | O | LEU | A | 49 | 765 | 1311 | 1996 | −13 | −306 | −381 | O |
| ATOM | 329 | CB | LEU | A | 49 | 17.449 | 9.482 | 16.054 | 1.00 | 16.07 | | C |
| ANISOU | 329 | CB | LEU | A | 49 | 1311 | 2369 | 2427 | 585 | −553 | −706 | C |
| ATOM | 330 | CG | LEU | A | 49 | 16.406 | 8.842 | 16.915 | 1.00 | 18.82 | | C |
| ANISOU | 330 | CG | LEU | A | 49 | 1807 | 3011 | 2333 | −131 | 107 | −1799 | C |
| ATOM | 331 | CD1 | LEU | A | 49 | 15.607 | 9.888 | 17.723 | 1.00 | 21.00 | | C |
| ANISOU | 331 | CD1 | LEU | A | 49 | 1856 | 2490 | 3635 | 201 | 193 | −1513 | C |
| ATOM | 332 | CD2 | LEU | A | 49 | 15.329 | 8.057 | 16.167 | 1.00 | 21.86 | | C |
| ANISOU | 332 | CD2 | LEU | A | 49 | 1123 | 4472 | 2712 | −39 | 122 | −2180 | C |
| ATOM | 333 | N | GLU | A | 50 | 19.863 | 8.664 | 17.478 | 1.00 | 12.59 | | N |
| ANISOU | 333 | N | GLU | A | 50 | 1632 | 1324 | 1828 | 423 | −640 | −390 | N |
| ATOM | 334 | CA | GLU | A | 50 | 20.794 | 8.115 | 18.429 | 1.00 | 12.64 | | C |
| ANISOU | 334 | CA | GLU | A | 50 | 1274 | 1725 | 1805 | 415 | −502 | −319 | C |
| ATOM | 335 | C | GLU | A | 50 | 20.459 | 8.676 | 19.802 | 1.00 | 12.62 | | C |
| ANISOU | 335 | C | GLU | A | 50 | 1339 | 1595 | 1863 | −8 | −665 | −519 | C |
| ATOM | 336 | O | GLU | A | 50 | 20.141 | 9.876 | 19.880 | 1.00 | 16.58 | | O |
| ANISOU | 336 | O | GLU | A | 50 | 2386 | 1855 | 2057 | 659 | −720 | −530 | O |
| ATOM | 337 | CB | GLU | A | 50 | 22.233 | 8.459 | 17.997 | 1.00 | 13.80 | | C |
| ANISOU | 337 | CB | GLU | A | 50 | 1489 | 1555 | 2199 | 126 | −416 | −181 | C |
| ATOM | 338 | CG | GLU | A | 50 | 23.263 | 7.640 | 18.743 | 1.00 | 15.20 | | C |
| ANISOU | 338 | CG | GLU | A | 50 | 1249 | 2059 | 2466 | 236 | −445 | −266 | C |
| ATOM | 339 | CD | GLU | A | 50 | 24.645 | 7.772 | 18.144 | 1.00 | 16.94 | | C |
| ANISOU | 339 | CD | GLU | A | 50 | 1227 | 2295 | 2915 | −409 | −537 | 784 | C |
| ATOM | 340 | OE1 | GLU | A | 50 | 24.788 | 8.195 | 16.989 | 1.00 | 20.13 | | O |
| ANISOU | 340 | OE1 | GLU | A | 50 | 1836 | 3275 | 2538 | 129 | −375 | 422 | O |
| ATOM | 341 | OE2 | GLU | A | 50 | 25.594 | 7.436 | 18.861 | 1.00 | 19.33 | | O |
| ANISOU | 341 | OE2 | GLU | A | 50 | 1315 | 3479 | 2549 | 244 | −530 | 125 | O |
| ATOM | 342 | N | VAL | A | 51 | 20.494 | 7.819 | 20.820 | 1.00 | 11.41 | | N |
| ANISOU | 342 | N | VAL | A | 51 | 886 | 1637 | 1810 | 27 | −256 | −533 | N |
| ATOM | 343 | CA | VAL | A | 51 | 20.141 | 8.221 | 22.169 | 1.00 | 12.45 | | C |
| ANISOU | 343 | CA | VAL | A | 51 | 828 | 1990 | 1913 | 26 | −159 | −634 | C |
| ATOM | 344 | C | VAL | A | 51 | 21.175 | 7.634 | 23.123 | 1.00 | 12.71 | | C |
| ANISOU | 344 | C | VAL | A | 51 | 1156 | 1967 | 1707 | 141 | −38 | −405 | C |
| ATOM | 345 | O | VAL | A | 51 | 21.393 | 6.424 | 23.124 | 1.00 | 13.44 | | O |
| ANISOU | 345 | O | VAL | A | 51 | 957 | 2029 | 2122 | 161 | 26 | −503 | O |
| ATOM | 346 | CB | VAL | A | 51 | 18.695 | 7.827 | 22.536 | 1.00 | 15.05 | | C |
| ANISOU | 346 | CB | VAL | A | 51 | 1021 | 2267 | 2429 | −65 | 133 | −149 | C |
| ATOM | 347 | CG1 | VAL | A | 51 | 18.386 | 8.372 | 23.921 | 1.00 | 24.80 | | C |
| ANISOU | 347 | CG1 | VAL | A | 51 | 1627 | 4560 | 3236 | −86 | 871 | −1193 | C |
| ATOM | 348 | CG2 | VAL | A | 51 | 17.693 | 8.358 | 21.531 | 1.00 | 22.42 | | C |
| ANISOU | 348 | CG2 | VAL | A | 51 | 1185 | 3028 | 4306 | 460 | −923 | −592 | C |
| ATOM | 349 | N | ASP | A | 52 | 21.806 | 8.497 | 23.928 | 1.00 | 13.43 | | N |
| ANISOU | 349 | N | ASP | A | 52 | 1169 | 2074 | 1860 | 548 | −407 | −658 | N |
| ATOM | 350 | CA | ASP | A | 52 | 22.868 | 8.080 | 24.832 | 1.00 | 14.32 | | C |
| ANISOU | 350 | CA | ASP | A | 52 | 969 | 2690 | 1780 | 590 | −129 | −229 | C |
| ATOM | 351 | C | ASP | A | 52 | 23.940 | 7.247 | 24.163 | 1.00 | 12.68 | | C |
| ANISOU | 351 | C | ASP | A | 52 | 888 | 2000 | 1929 | 270 | −133 | −313 | C |
| ATOM | 352 | O | ASP | A | 52 | 24.473 | 6.302 | 24.776 | 1.00 | 12.86 | | O |
| ANISOU | 352 | O | ASP | A | 52 | 1050 | 2059 | 1779 | 289 | −255 | −365 | O |
| ATOM | 353 | CB | ASP | A | 52 | 22.313 | 7.279 | 26.031 | 1.00 | 15.23 | | C |
| ANISOU | 353 | CB | ASP | A | 52 | 1485 | 2260 | 2043 | 588 | 153 | −250 | C |
| ATOM | 354 | CG | ASP | A | 52 | 21.221 | 8.033 | 26.752 | 1.00 | 15.85 | | C |
| ANISOU | 354 | CG | ASP | A | 52 | 1251 | 2482 | 2288 | 452 | 202 | −432 | C |
| ATOM | 355 | OD1 | ASP | A | 52 | 21.335 | 9.280 | 26.852 | 1.00 | 19.04 | | O |
| ANISOU | 355 | OD1 | ASP | A | 52 | 2064 | 2364 | 2809 | 889 | −212 | −475 | O |
| ATOM | 356 | OD2 | ASP | A | 52 | 20.265 | 7.375 | 27.254 | 1.00 | 22.22 | | O |
| ANISOU | 356 | OD2 | ASP | A | 52 | 1951 | 3488 | 3003 | 388 | 855 | 254 | O |
| ATOM | 357 | N | GLY | A | 53 | 24.256 | 7.590 | 22.920 | 1.00 | 11.56 | | N |
| ANISOU | 357 | N | GLY | A | 53 | 873 | 1827 | 1693 | 224 | −297 | −544 | N |
| ATOM | 358 | CA | GLY | A | 53 | 25.303 | 6.898 | 22.157 | 1.00 | 12.16 | | C |
| ANISOU | 358 | CA | GLY | A | 53 | 841 | 1854 | 1924 | 34 | −172 | −665 | C |
| ATOM | 359 | C | GLY | A | 53 | 24.893 | 5.590 | 21.540 | 1.00 | 10.74 | | C |
| ANISOU | 359 | C | GLY | A | 53 | 876 | 1587 | 1619 | 102 | −14 | −373 | C |
| ATOM | 360 | O | GLY | A | 53 | 25.722 | 4.876 | 20.970 | 1.00 | 13.23 | | O |
| ANISOU | 360 | O | GLY | A | 53 | 1075 | 1841 | 2111 | 135 | 207 | −588 | O |
| ATOM | 361 | N | HIS | A | 54 | 23.588 | 5.278 | 21.627 | 1.00 | 10.45 | | N |
| ANISOU | 361 | N | HIS | A | 54 | 923 | 1462 | 1584 | 46 | −170 | −319 | N |
| ATOM | 362 | CA | HIS | A | 54 | 23.067 | 4.052 | 21.057 | 1.00 | 11.15 | | C |
| ANISOU | 362 | CA | HIS | A | 54 | 1260 | 1190 | 1787 | 40 | −329 | −19 | C |
| ATOM | 363 | C | HIS | A | 54 | 22.272 | 4.348 | 19.796 | 1.00 | 11.49 | | C |
| ANISOU | 363 | C | HIS | A | 54 | 1152 | 1268 | 1945 | 162 | −518 | −438 | C |
| ATOM | 364 | O | HIS | A | 54 | 21.439 | 5.261 | 19.824 | 1.00 | 13.79 | | O |
| ANISOU | 364 | O | HIS | A | 54 | 1248 | 1499 | 2493 | 410 | −778 | −635 | O |
| ATOM | 365 | CB | HIS | A | 54 | 22.131 | 3.336 | 22.018 | 1.00 | 15.21 | | C |
| ANISOU | 365 | CB | HIS | A | 54 | 1371 | 2002 | 2407 | −400 | −200 | 188 | C |
| ATOM | 366 | CG | HIS | A | 54 | 22.754 | 2.777 | 23.251 | 1.00 | 18.86 | | C |
| ANISOU | 366 | CG | HIS | A | 54 | 1871 | 3036 | 2257 | −344 | 139 | 765 | C |
| ATOM | 367 | ND1 | HIS | A | 54 | 23.573 | 3.446 | 24.125 | 1.00 | 21.63 | | N |
| ANISOU | 367 | ND1 | HIS | A | 54 | 2992 | 3025 | 2201 | 450 | −640 | 437 | N |
| ATOM | 368 | CD2 | HIS | A | 54 | 22.659 | 1.522 | 23.737 | 1.00 | 28.34 | | C |
| ANISOU | 368 | CD2 | HIS | A | 54 | 4707 | 3278 | 2781 | −603 | −583 | 1178 | C |
| ATOM | 369 | CE1 | HIS | A | 54 | 23.961 | 2.661 | 25.112 | 1.00 | 23.49 | | C |
| ANISOU | 369 | CE1 | HIS | A | 54 | 3693 | 3419 | 1815 | 428 | −113 | 703 | C |
| ATOM | 370 | NE2 | HIS | A | 54 | 23.412 | 1.485 | 24.894 | 1.00 | 31.57 | | N |
| ANISOU | 370 | NE2 | HIS | A | 54 | 5214 | 3434 | 3346 | 66 | −1157 | 1118 | N |
| ATOM | 371 | N | PHE | A | 55 | 22.539 | 3.594 | 18.729 | 1.00 | 12.79 | | N |
| ANISOU | 371 | N | PHE | A | 55 | 1077 | 1770 | 2013 | 378 | −552 | −581 | N |
| ATOM | 372 | CA | PHE | A | 55 | 21.741 | 3.726 | 17.533 | 1.00 | 12.66 | | C |
| ANISOU | 372 | CA | PHE | A | 55 | 1060 | 1682 | 2068 | 379 | −617 | −657 | C |
| ATOM | 373 | C | PHE | A | 55 | 20.327 | 3.246 | 17.746 | 1.00 | 14.36 | | C |
| ANISOU | 373 | C | PHE | A | 55 | 1219 | 2307 | 1929 | 62 | −666 | −166 | C |
| ATOM | 374 | O | PHE | A | 55 | 20.064 | 2.189 | 18.348 | 1.00 | 18.48 | | O |
| ANISOU | 374 | O | PHE | A | 55 | 1877 | 2501 | 2642 | −278 | −1035 | 179 | O |
| ATOM | 375 | CB | PHE | A | 55 | 22.369 | 2.889 | 16.443 | 1.00 | 12.45 | | C |
| ANISOU | 375 | CB | PHE | A | 55 | 1390 | 1311 | 2029 | 109 | −543 | −567 | C |
| ATOM | 376 | CG | PHE | A | 55 | 23.811 | 3.216 | 16.115 | 1.00 | 12.94 | | C |
| ANISOU | 376 | CG | PHE | A | 55 | 1409 | 1721 | 1788 | 178 | −334 | −514 | C |
| ATOM | 377 | CD1 | PHE | A | 55 | 24.354 | 4.451 | 16.347 | 1.00 | 13.10 | | C |
| ANISOU | 377 | CD1 | PHE | A | 55 | 1237 | 1867 | 1873 | 9 | −275 | −440 | C |
| ATOM | 378 | CD2 | PHE | A | 55 | 24.592 | 2.222 | 15.554 | 1.00 | 15.50 | | C |
| ANISOU | 378 | CD2 | PHE | A | 55 | 2015 | 2120 | 1753 | 265 | 172 | −674 | C |
| ATOM | 379 | CE1 | PHE | A | 55 | 25.676 | 4.721 | 16.017 | 1.00 | 14.30 | | C |
| ANISOU | 379 | CE1 | PHE | A | 55 | 1240 | 2248 | 1946 | 79 | −237 | −496 | C |
| ATOM | 380 | CE2 | PHE | A | 55 | 25.891 | 2.484 | 15.215 | 1.00 | 17.11 | | C |
| ANISOU | 380 | CE2 | PHE | A | 55 | 1788 | 2666 | 2048 | 359 | −11 | −967 | C |
| ATOM | 381 | CZ | PHE | A | 55 | 26.440 | 3.743 | 15.445 | 1.00 | 15.54 | | C |
| ANISOU | 381 | CZ | PHE | A | 55 | 1372 | 2675 | 1858 | 405 | −359 | −567 | C |
| ATOM | 382 | N | VAL | A | 56 | 19.392 | 4.058 | 17.247 | 1.00 | 13.42 | | N |
| ANISOU | 382 | N | VAL | A | 56 | 874 | 1949 | 2277 | −90 | −573 | −412 | N |
| ATOM | 383 | CA | VAL | A | 56 | 17.999 | 3.605 | 17.317 | 1.00 | 15.95 | | C |
| ANISOU | 383 | CA | VAL | A | 56 | 988 | 2930 | 2144 | −478 | −569 | 16 | C |
| ATOM | 384 | C | VAL | A | 56 | 17.312 | 4.015 | 16.011 | 1.00 | 12.09 | | C |
| ANISOU | 384 | C | VAL | A | 56 | 645 | 2118 | 1833 | −125 | −183 | −278 | C |
| ATOM | 385 | O | VAL | A | 56 | 17.774 | 4.863 | 15.256 | 1.00 | 11.89 | | O |
| ANISOU | 385 | O | VAL | A | 56 | 676 | 1814 | 2027 | −193 | −189 | −395 | O |
| ATOM | 386 | CB | VAL | A | 56 | 17.242 | 4.163 | 18.507 | 1.00 | 19.54 | | C |
| ANISOU | 386 | CB | VAL | A | 56 | 1064 | 4577 | 1782 | −736 | −466 | 24 | C |
| ATOM | 387 | CG1 | VAL | A | 56 | 17.887 | 3.841 | 19.857 | 1.00 | 39.57 | | C |
| ANISOU | 387 | CG1 | VAL | A | 56 | 4497 | 8588 | 1948 | −624 | −1600 | 335 | C |
| ATOM | 388 | CG2 | VAL | A | 56 | 17.082 | 5.659 | 18.379 | 1.00 | 23.13 | | C |
| ANISOU | 388 | CG2 | VAL | A | 56 | 2060 | 4151 | 2578 | −917 | −234 | −1646 | C |
| ATOM | 389 | N | THR | A | 57 | 16.179 | 3.368 | 15.755 | 1.00 | 10.77 | | N |
| ANISOU | 389 | N | THR | A | 57 | 591 | 1586 | 1915 | 73 | −274 | −16 | N |
| ATOM | 390 | CA | THR | A | 57 | 15.344 | 3.722 | 14.622 | 1.00 | 9.66 | | C |
| ANISOU | 390 | CA | THR | A | 57 | 657 | 1755 | 1257 | −36 | −7 | −106 | C |
| ATOM | 391 | C | THR | A | 57 | 13.907 | 3.950 | 15.125 | 1.00 | 9.83 | | C |
| ANISOU | 391 | C | THR | A | 57 | 510 | 1789 | 1435 | −91 | −123 | −312 | C |
| ATOM | 392 | O | THR | A | 57 | 13.433 | 3.134 | 15.907 | 1.00 | 12.11 | | O |
| ANISOU | 392 | O | THR | A | 57 | 866 | 1852 | 1885 | −219 | 190 | −237 | O |
| ATOM | 393 | CB | THR | A | 57 | 15.359 | 2.626 | 13.531 | 1.00 | 13.09 | | C |
| ANISOU | 393 | CB | THR | A | 57 | 965 | 2004 | 2004 | 342 | −141 | −567 | C |
| ATOM | 394 | OG1 | THR | A | 57 | 16.718 | 2.432 | 13.096 | 1.00 | 15.91 | | O |
| ANISOU | 394 | OG1 | THR | A | 57 | 1124 | 2250 | 2672 | 240 | 190 | −897 | O |
| ATOM | 395 | CG2 | THR | A | 57 | 14.532 | 3.027 | 12.326 | 1.00 | 15.76 | | C |
| ANISOU | 395 | CG2 | THR | A | 57 | 1202 | 3573 | 1212 | −43 | 57 | −633 | C |
| ATOM | 396 | N | MET | A | 58 | 13.247 | 5.022 | 14.687 | 1.00 | 10.69 | | N |
| ANISOU | 396 | N | MET | A | 58 | 571 | 1910 | 1583 | −59 | −326 | −345 | N |
| ATOM | 397 | CA | MET | A | 58 | 11.878 | 5.306 | 14.998 | 1.00 | 10.91 | | C |
| ANISOU | 397 | CA | MET | A | 58 | 545 | 2172 | 1430 | −67 | −393 | −257 | C |
| ATOM | 398 | C | MET | A | 58 | 11.022 | 5.026 | 13.768 | 1.00 | 9.24 | | C |
| ANISOU | 398 | C | MET | A | 58 | 573 | 1661 | 1275 | 123 | −335 | −44 | C |
| ATOM | 399 | O | MET | A | 58 | 11.391 | 5.410 | 12.666 | 1.00 | 12.41 | | O |
| ANISOU | 399 | O | MET | A | 58 | 931 | 2471 | 1313 | −206 | −152 | −16 | O |
| ATOM | 400 | CB | MET | A | 58 | 11.700 | 6.777 | 15.372 | 1.00 | 23.02 | | C |
| ANISOU | 400 | CB | MET | A | 58 | 2668 | 3389 | 2689 | 1351 | −1296 | −2041 | C |
| ATOM | 401 | CG | MET | A | 58 | 10.398 | 7.215 | 15.921 | 1.00 | 21.87 | | C |
| ANISOU | 401 | CG | MET | A | 58 | 2794 | 2660 | 2856 | 916 | −597 | −1062 | C |
| ATOM | 402 | SD | MET | A | 58 | 10.386 | 9.021 | 16.022 | 1.00 | 20.40 | | S |
| ANISOU | 402 | SD | MET | A | 58 | 2545 | 2504 | 2704 | 830 | −622 | −698 | S |
| ATOM | 403 | CE | MET | A | 58 | 12.028 | 9.442 | 16.457 | 1.00 | 20.71 | | C |
| ANISOU | 403 | CE | MET | A | 58 | 2313 | 2481 | 3075 | 699 | −356 | 387 | C |
| ATOM | 404 | N | GLN | A | 59 | 9.887 | 4.394 | 13.977 | 1.00 | 10.30 | | N |
| ANISOU | 404 | N | GLN | A | 59 | 718 | 1808 | 1386 | −130 | −319 | −106 | N |
| ATOM | 405 | CA | GLN | A | 59 | 8.931 | 4.156 | 12.921 | 1.00 | 9.13 | | C |
| ANISOU | 405 | CA | GLN | A | 59 | 677 | 1636 | 1155 | 59 | −133 | −472 | C |
| ATOM | 406 | C | GLN | A | 59 | 7.639 | 4.909 | 13.316 | 1.00 | 9.42 | | C |
| ANISOU | 406 | C | GLN | A | 59 | 716 | 1513 | 1350 | 105 | −58 | −246 | C |
| ATOM | 407 | O | GLN | A | 59 | 7.011 | 4.592 | 14.306 | 1.00 | 9.81 | | O |
| ANISOU | 407 | O | GLN | A | 59 | 784 | 1571 | 1372 | 201 | −15 | −170 | O |
| ATOM | 408 | CB | GLN | A | 59 | 8.689 | 2.678 | 12.793 | 1.00 | 10.75 | | C |
| ANISOU | 408 | CB | GLN | A | 59 | 905 | 1715 | 1464 | 130 | −87 | −620 | C |
| ATOM | 409 | CG | GLN | A | 59 | 7.824 | 2.338 | 11.567 | 1.00 | 10.87 | | C |
| ANISOU | 409 | CG | GLN | A | 59 | 762 | 1605 | 1764 | −268 | −208 | −472 | C |
| ATOM | 410 | CD | GLN | A | 59 | 7.882 | 0.848 | 11.400 | 1.00 | 11.68 | | C |
| ANISOU | 410 | CD | GLN | A | 59 | 1456 | 1519 | 1461 | −57 | −143 | −441 | C |
| ATOM | 411 | OE1 | GLN | A | 59 | 8.691 | 0.200 | 10.732 | 1.00 | 15.35 | | O |
| ANISOU | 411 | OE1 | GLN | A | 59 | 1502 | 2447 | 1882 | 236 | −47 | −1026 | O |
| ATOM | 412 | NE2 | GLN | A | 59 | 7.047 | 0.102 | 12.070 | 1.00 | 10.81 | | N |
| ANISOU | 412 | NE2 | GLN | A | 59 | 1133 | 1327 | 1648 | 75 | −377 | −314 | N |
| ATOM | 413 | N | ILE | A | 60 | 7.312 | 5.901 | 12.504 | 1.00 | 9.09 | | N |
| ANISOU | 413 | N | ILE | A | 60 | 687 | 1502 | 1266 | −37 | −55 | −274 | N |
| ATOM | 414 | CA | ILE | A | 60 | 6.274 | 6.852 | 12.770 | 1.00 | 9.43 | | C |
| ANISOU | 414 | CA | ILE | A | 60 | 828 | 1389 | 1367 | 43 | −218 | −98 | C |
| ATOM | 415 | C | ILE | A | 60 | 5.065 | 6.504 | 11.934 | 1.00 | 8.41 | | C |
| ANISOU | 415 | C | ILE | A | 60 | 751 | 1195 | 1250 | 107 | −45 | −362 | C |
| ATOM | 416 | O | ILE | A | 60 | 5.140 | 6.500 | 10.695 | 1.00 | 10.24 | | O |
| ANISOU | 416 | O | ILE | A | 60 | 809 | 1768 | 1313 | −254 | 64 | −563 | O |
| ATOM | 417 | CB | ILE | A | 60 | 6.740 | 8.284 | 12.480 | 1.00 | 10.13 | | C |
| ANISOU | 417 | CB | ILE | A | 60 | 908 | 1446 | 1495 | −51 | −560 | 33 | C |
| ATOM | 418 | CG1 | ILE | A | 60 | 8.046 | 8.630 | 13.176 | 1.00 | 12.71 | | C |
| ANISOU | 418 | CG1 | ILE | A | 60 | 1278 | 1682 | 1870 | −235 | −898 | 49 | C |
| ATOM | 419 | CG2 | ILE | A | 60 | 5.608 | 9.247 | 12.826 | 1.00 | 11.47 | | C |
| ANISOU | 419 | CG2 | ILE | A | 60 | 1316 | 1437 | 1606 | 101 | −490 | −228 | C |
| ATOM | 420 | CD1 | ILE | A | 60 | 8.826 | 9.772 | 12.524 | 1.00 | 23.60 | | C |
| ANISOU | 420 | CD1 | ILE | A | 60 | 2584 | 4054 | 2331 | −2178 | −528 | 202 | C |
| ATOM | 421 | N | TRP | A | 61 | 3.941 | 6.232 | 12.581 | 1.00 | 8.42 | | N |
| ANISOU | 421 | N | TRP | A | 61 | 781 | 1412 | 1006 | 82 | −138 | −426 | N |
| ATOM | 422 | CA | TRP | A | 61 | 2.681 | 5.866 | 11.962 | 1.00 | 9.63 | | C |
| ANISOU | 422 | CA | TRP | A | 61 | 795 | 1560 | 1306 | 34 | −155 | −800 | C |
| ATOM | 423 | C | TRP | A | 61 | 1.662 | 6.972 | 11.996 | 1.00 | 9.29 | | C |
| ANISOU | 423 | C | TRP | A | 61 | 639 | 1723 | 1169 | 54 | −97 | −728 | C |
| ATOM | 424 | O | TRP | A | 61 | 1.430 | 7.603 | 13.026 | 1.00 | 12.75 | | O |
| ANISOU | 424 | O | TRP | A | 61 | 1303 | 2462 | 1080 | 776 | −276 | −851 | O |
| ATOM | 425 | CB | TRP | A | 61 | 2.109 | 4.646 | 12.685 | 1.00 | 12.27 | | C |
| ANISOU | 425 | CB | TRP | A | 61 | 1027 | 1566 | 2069 | −160 | −139 | −677 | C |
| ATOM | 426 | CG | TRP | A | 61 | 2.804 | 3.366 | 12.426 | 1.00 | 9.69 | | C |
| ANISOU | 426 | CG | TRP | A | 61 | 853 | 1657 | 1172 | −153 | −47 | −613 | C |
| ATOM | 427 | CD1 | TRP | A | 61 | 4.055 | 3.074 | 12.833 | 1.00 | 9.85 | | C |
| ANISOU | 427 | CD1 | TRP | A | 61 | 1116 | 1596 | 1032 | −134 | −222 | −617 | C |
| ATOM | 428 | CD2 | TRP | A | 61 | 2.274 | 2.229 | 11.723 | 1.00 | 10.00 | | C |
| ANISOU | 428 | CD2 | TRP | A | 61 | 756 | 1590 | 1452 | −206 | 60 | −594 | C |
| ATOM | 429 | NE1 | TRP | A | 61 | 4.229 | 1.855 | 12.391 | 1.00 | 10.25 | | N |
| ANISOU | 429 | NE1 | TRP | A | 61 | 1113 | 1509 | 1272 | −139 | −290 | −351 | N |
| ATOM | 430 | CE2 | TRP | A | 61 | 3.264 | 1.227 | 11.707 | 1.00 | 8.98 | | C |
| ANISOU | 430 | CE2 | TRP | A | 61 | 885 | 1428 | 1098 | −191 | −81 | −408 | C |
| ATOM | 431 | CE3 | TRP | A | 61 | 1.069 | 1.938 | 11.078 | 1.00 | 10.92 | | C |
| ANISOU | 431 | CE3 | TRP | A | 61 | 909 | 1681 | 1560 | −128 | −182 | −609 | C |
| ATOM | 432 | CZ2 | TRP | A | 61 | 3.151 | −0.027 | 11.119 | 1.00 | 11.46 | | C |
| ANISOU | 432 | CZ2 | TRP | A | 61 | 1161 | 1613 | 1582 | −58 | −284 | −659 | C |
| ATOM | 433 | CZ3 | TRP | A | 61 | 0.970 | 0.707 | 10.511 | 1.00 | 11.08 | | C |
| ANISOU | 433 | CZ3 | TRP | A | 61 | 923 | 1951 | 1336 | −89 | 26 | −909 | C |
| ATOM | 434 | CH2 | TRP | A | 61 | 1.938 | −0.283 | 10.504 | 1.00 | 11.97 | | C |
| ANISOU | 434 | CH2 | TRP | A | 61 | 901 | 2008 | 1638 | −61 | −60 | −1009 | C |
| ATOM | 435 | N | ASP | A | 62 | 1.051 | 7.190 | 10.861 | 1.00 | 8.89 | | N |
| ANISOU | 435 | N | ASP | A | 62 | 793 | 1512 | 1074 | −2 | −51 | −644 | N |
| ATOM | 436 | CA | ASP | A | 62 | −0.078 | 8.069 | 10.742 | 1.00 | 10.21 | | C |
| ANISOU | 436 | CA | ASP | A | 62 | 983 | 1457 | 1438 | 99 | −193 | −567 | C |
| ATOM | 437 | C | ASP | A | 62 | −1.237 | 7.147 | 10.324 | 1.00 | 12.06 | | C |
| ANISOU | 437 | C | ASP | A | 62 | 578 | 2115 | 1889 | 115 | 112 | −1050 | C |
| ATOM | 438 | O | ASP | A | 62 | −1.247 | 6.753 | 9.146 | 1.00 | 12.16 | | O |
| ANISOU | 438 | O | ASP | A | 62 | 985 | 2109 | 1526 | −304 | −192 | −415 | O |
| ATOM | 439 | CB | ASP | A | 62 | −0.084 | 8.988 | 9.589 | 1.00 | 15.59 | | C |
| ANISOU | 439 | CB | ASP | A | 62 | 2257 | 1741 | 1925 | 257 | −401 | −203 | C |
| ATOM | 440 | CG | ASP | A | 62 | −0.937 | 10.199 | 9.534 | 1.00 | 13.89 | | C |
| ANISOU | 440 | CG | ASP | A | 62 | 1669 | 1686 | 1924 | 94 | −267 | −67 | C |
| ATOM | 441 | OD1 | ASP | A | 62 | −1.968 | 10.045 | 10.177 | 1.00 | 16.99 | | O |
| ANISOU | 441 | OD1 | ASP | A | 62 | 2207 | 2641 | 1608 | 231 | 127 | −87 | O |
| ATOM | 442 | OD2 | ASP | A | 62 | −0.647 | 11.111 | 8.779 | 1.00 | 18.11 | | O |
| ANISOU | 442 | OD2 | ASP | A | 62 | 1918 | 2087 | 2875 | 128 | −47 | 536 | O |
| ATOM | 443 | N | THR | A | 63 | −2.079 | 6.876 | 11.298 | 1.00 | 14.02 | | N |
| ANISOU | 443 | N | THR | A | 63 | 1250 | 1927 | 2151 | −286 | 496 | −1051 | N |
| ATOM | 444 | CA | THR | A | 63 | −3.105 | 5.846 | 11.073 | 1.00 | 13.42 | | C |
| ANISOU | 444 | CA | THR | A | 63 | 1293 | 1404 | 2401 | −89 | −133 | −505 | C |
| ATOM | 445 | C | THR | A | 63 | −4.448 | 6.361 | 10.625 | 1.00 | 13.18 | | C |
| ANISOU | 445 | C | THR | A | 63 | 1331 | 1731 | 1946 | −105 | −43 | −449 | C |
| ATOM | 446 | O | THR | A | 63 | −5.394 | 5.576 | 10.467 | 1.00 | 15.79 | | O |
| ANISOU | 446 | O | THR | A | 63 | 1305 | 2352 | 2344 | −450 | 100 | −818 | O |
| ATOM | 447 | CB | THR | A | 63 | −3.312 | 5.061 | 12.384 | 1.00 | 13.58 | | C |
| ANISOU | 447 | CB | THR | A | 63 | 1212 | 1452 | 2498 | −78 | −368 | −452 | C |
| ATOM | 448 | OG1 | THR | A | 63 | −3.325 | 5.945 | 13.530 | 1.00 | 15.93 | | O |
| ANISOU | 448 | OG1 | THR | A | 63 | 2154 | 1477 | 2422 | 82 | 62 | −415 | O |
| ATOM | 449 | CG2 | THR | A | 63 | −2.135 | 4.104 | 12.540 | 1.00 | 16.13 | | C |
| ANISOU | 449 | CG2 | THR | A | 63 | 1619 | 2078 | 2432 | 490 | −206 | −403 | C |
| ATOM | 450 | N | ALA | A | 64 | −4.534 | 7.647 | 10.380 | 1.00 | 18.74 | | N |
| ANISOU | 450 | N | ALA | A | 64 | 1726 | 1846 | 3548 | 45 | −1286 | −322 | N |
| ATOM | 451 | CA | ALA | A | 64 | −5.670 | 8.339 | 9.835 | 1.00 | 28.21 | | C |
| ANISOU | 451 | CA | ALA | A | 64 | 2654 | 3165 | 4898 | 1570 | −1878 | −1214 | C |
| ATOM | 452 | C | ALA | A | 64 | −5.869 | 7.982 | 8.355 | 1.00 | 38.41 | | C |
| ANISOU | 452 | C | ALA | A | 64 | 4300 | 5157 | 5135 | 1966 | −3360 | −1295 | C |
| ATOM | 453 | O | ALA | A | 64 | −5.755 | 8.834 | 7.471 | 1.00 | 45.74 | | O |
| ANISOU | 453 | O | ALA | A | 64 | 6460 | 6481 | 4440 | 1137 | 12 | −1572 | O |
| ATOM | 454 | CB | ALA | A | 64 | −5.526 | 9.845 | 9.944 | 1.00 | 36.87 | | C |
| ANISOU | 454 | CB | ALA | A | 64 | 4762 | 2993 | 6254 | 1431 | −4164 | −264 | C |
| ATOM | 455 | N | GLY | A | 65 | −6.175 | 6.757 | 7.999 | 1.00 | 45.31 | | N |
| ANISOU | 455 | N | GLY | A | 65 | 6254 | 5934 | 5027 | 1025 | −3063 | −2024 | N |
| ATOM | 456 | CA | GLY | A | 65 | −6.626 | 6.543 | 6.630 | 1.00 | 37.35 | | C |
| ANISOU | 456 | CA | GLY | A | 65 | 5717 | 4595 | 3880 | 601 | −1665 | −770 | C |
| ATOM | 457 | C | GLY | A | 65 | −8.010 | 7.163 | 6.393 | 1.00 | 33.70 | | C |
| ANISOU | 457 | C | GLY | A | 65 | 5097 | 3994 | 3713 | −371 | −1736 | −295 | C |
| ATOM | 458 | O | GLY | A | 65 | −8.214 | 8.320 | 6.756 | 1.00 | 24.96 | | O |
| ANISOU | 458 | O | GLY | A | 65 | 3145 | 3483 | 2855 | −1113 | −573 | 559 | O |
| ATOM | 459 | N | GLN | A | 66 | −8.933 | 6.409 | 5.828 | 1.00 | 31.21 | | N |
| ANISOU | 459 | N | GLN | A | 66 | 4882 | 4628 | 2346 | −1362 | 153 | −821 | N |
| ATOM | 460 | CA | GLN | A | 66 | −10.298 | 6.681 | 5.436 | 1.00 | 27.98 | | C |
| ANISOU | 460 | CA | GLN | A | 66 | 5028 | 3301 | 2304 | −1931 | −418 | −512 | C |
| ATOM | 461 | C | GLN | A | 66 | −11.314 | 6.528 | 6.556 | 1.00 | 28.61 | | C |
| ANISOU | 461 | C | GLN | A | 66 | 4921 | 2883 | 3066 | −183 | 135 | −250 | C |
| ATOM | 462 | O | GLN | A | 66 | −11.392 | 5.430 | 7.124 | 1.00 | 21.52 | | O |
| ANISOU | 462 | O | GLN | A | 66 | 3660 | 2590 | 1929 | −361 | 65 | −827 | O |
| ATOM | 463 | CB | GLN | A | 66 | −10.631 | 5.645 | 4.310 | 1.00 | 32.60 | | C |
| ANISOU | 463 | CB | GLN | A | 66 | 6531 | 3483 | 2371 | −2119 | −371 | −662 | C |
| ATOM | 464 | CG | GLN | A | 66 | −9.749 | 5.912 | 3.106 | 1.00 | 31.31 | | C |
| ANISOU | 464 | CG | GLN | A | 66 | 4063 | 4559 | 3273 | −957 | −70 | −1599 | C |
| ATOM | 465 | CD | GLN | A | 66 | −9.401 | 4.752 | 2.205 | 1.00 | 38.05 | | C |
| ANISOU | 465 | CD | GLN | A | 66 | 3525 | 6128 | 4802 | −524 | −1433 | −3346 | C |
| ATOM | 466 | OE1 | GLN | A | 66 | −10.095 | 4.338 | 1.286 | 1.00 | 30.80 | | O |
| ANISOU | 466 | OE1 | GLN | A | 66 | 5274 | 4265 | 2162 | −1998 | −1048 | −444 | O |
| ATOM | 467 | NE2 | GLN | A | 66 | −8.200 | 4.261 | 2.457 | 1.00 | 47.00 | | N |
| ANISOU | 467 | NE2 | GLN | A | 66 | 4737 | 7090 | 6030 | 864 | −1990 | −3601 | N |
| ATOM | 468 | N | GLU | A | 67 | −12.114 | 7.518 | 6.954 | 1.00 | 33.62 | | N |
| ANISOU | 468 | N | GLU | A | 67 | 5800 | 3235 | 3739 | 784 | −775 | −346 | N |
| ATOM | 469 | CA | GLU | A | 67 | −12.986 | 7.285 | 8.113 | 1.00 | 37.64 | | C |
| ANISOU | 469 | CA | GLU | A | 67 | 5249 | 4573 | 4480 | 1113 | −87 | −1555 | C |
| ATOM | 470 | C | GLU | A | 67 | −13.956 | 6.117 | 7.959 | 1.00 | 30.18 | | C |
| ANISOU | 470 | C | GLU | A | 67 | 3422 | 4686 | 3360 | 1764 | −673 | −617 | C |
| ATOM | 471 | O | GLU | A | 67 | −14.211 | 5.432 | 8.963 | 1.00 | 26.53 | | O |
| ANISOU | 471 | O | GLU | A | 67 | 2976 | 4876 | 2227 | 1961 | −169 | −1539 | O |
| ATOM | 472 | CB | GLU | A | 67 | −13.828 | 8.519 | 8.480 | 1.00 | 47.71 | | C |
| ANISOU | 472 | CB | GLU | A | 67 | 6146 | 5167 | 6815 | 1946 | −663 | −2132 | C |
| ATOM | 473 | CG | GLU | A | 67 | −15.321 | 8.281 | 8.284 | 1.00 | 53.97 | | C |
| ANISOU | 473 | CG | GLU | A | 67 | 5906 | 6301 | 8299 | 2164 | −132 | −2712 | C |
| ATOM | 474 | CD | GLU | A | 67 | −16.171 | 8.179 | 9.530 | 1.00 | 61.49 | | C |
| ANISOU | 474 | CD | GLU | A | 67 | 6956 | 7591 | 8818 | 1078 | 573 | −2968 | C |
| ATOM | 475 | OE1 | GLU | A | 67 | −16.858 | 9.175 | 9.878 | 1.00 | 57.77 | | O |
| ANISOU | 475 | OE1 | GLU | A | 67 | 7250 | 5599 | 9102 | −518 | 2668 | −2034 | O |
| ATOM | 476 | OE2 | GLU | A | 67 | −16.190 | 7.106 | 10.179 | 1.00 | 54.96 | | O |
| ANISOU | 476 | OE2 | GLU | A | 67 | 4806 | 8313 | 7765 | 3189 | 443 | −2617 | O |
| ATOM | 477 | N | ARG | A | 68 | −14.499 | 5.826 | 6.766 | 1.00 | 26.19 | | N |
| ANISOU | 477 | N | ARG | A | 68 | 3276 | 4131 | 2545 | 1880 | 126 | −396 | N |
| ATOM | 478 | CA | ARG | A | 68 | −15.400 | 4.673 | 6.743 | 1.00 | 24.85 | | C |
| ANISOU | 478 | CA | ARG | A | 68 | 1807 | 5120 | 2513 | 1831 | 135 | −436 | C |
| ATOM | 479 | C | ARG | A | 68 | −14.666 | 3.381 | 7.059 | 1.00 | 20.08 | | C |
| ANISOU | 479 | C | ARG | A | 68 | 1166 | 4373 | 2093 | 767 | 11 | 48 | C |
| ATOM | 480 | O | ARG | A | 68 | −15.294 | 2.374 | 7.367 | 1.00 | 23.37 | | O |
| ANISOU | 480 | O | ARG | A | 68 | 1341 | 4907 | 2632 | 55 | 195 | −534 | O |
| ATOM | 481 | CB | ARG | A | 68 | −16.169 | 4.504 | 5.423 | 1.00 | 27.25 | | C |
| ANISOU | 481 | CB | ARG | A | 68 | 2628 | 5057 | 2670 | 1883 | −320 | 144 | C |
| ATOM | 482 | CG | ARG | A | 68 | −15.289 | 4.120 | 4.270 | 1.00 | 27.91 | | C |
| ANISOU | 482 | CG | ARG | A | 68 | 3331 | 5163 | 2111 | 743 | 49 | 294 | C |
| ATOM | 483 | CD | ARG | A | 68 | −15.926 | 4.395 | 2.913 | 1.00 | 32.47 | | C |
| ANISOU | 483 | CD | ARG | A | 68 | 4593 | 5186 | 2559 | 927 | −877 | −152 | C |
| ATOM | 484 | NE | ARG | A | 68 | −16.472 | 3.146 | 2.371 | 1.00 | 30.67 | | N |
| ANISOU | 484 | NE | ARG | A | 68 | 3509 | 5306 | 2837 | 1641 | 219 | −1285 | N |
| ATOM | 485 | CZ | ARG | A | 68 | −17.156 | 3.091 | 1.238 | 1.00 | 35.40 | | C |
| ANISOU | 485 | CZ | ARG | A | 68 | 5308 | 4922 | 3221 | 385 | −667 | −549 | C |
| ATOM | 486 | NH1 | ARG | A | 68 | −17.351 | 4.236 | 0.598 | 1.00 | 32.90 | | N |
| ANISOU | 486 | NH1 | ARG | A | 68 | 2810 | 5259 | 4433 | −756 | −526 | 393 | N |
| ATOM | 487 | NH2 | ARG | A | 68 | −17.624 | 1.936 | 0.797 | 1.00 | 48.53 | | N |
| ANISOU | 487 | NH2 | ARG | A | 68 | 10025 | 4272 | 4143 | 1740 | −2399 | −1541 | N |
| ATOM | 488 | N | PHE | A | 69 | −13.355 | 3.349 | 6.988 | 1.00 | 16.60 | | N |
| ANISOU | 488 | N | PHE | A | 69 | 1135 | 3031 | 2139 | 528 | −382 | −70 | N |
| ATOM | 489 | CA | PHE | A | 69 | −12.570 | 2.163 | 7.295 | 1.00 | 12.96 | | C |
| ANISOU | 489 | CA | PHE | A | 69 | 1138 | 2325 | 1461 | 159 | −179 | −408 | C |
| ATOM | 490 | C | PHE | A | 69 | −11.822 | 2.250 | 8.630 | 1.00 | 11.78 | | C |
| ANISOU | 490 | C | PHE | A | 69 | 1187 | 1698 | 1591 | −5 | −319 | −378 | C |
| ATOM | 491 | O | PHE | A | 69 | −10.847 | 1.542 | 8.855 | 1.00 | 13.83 | | O |
| ANISOU | 491 | O | PHE | A | 69 | 1114 | 2416 | 1723 | 274 | −294 | −486 | O |
| ATOM | 492 | CB | PHE | A | 69 | −11.572 | 1.860 | 6.167 | 1.00 | 13.15 | | C |
| ANISOU | 492 | CB | PHE | A | 69 | 980 | 2319 | 1698 | −393 | 95 | −448 | C |
| ATOM | 493 | CG | PHE | A | 69 | −12.273 | 1.469 | 4.874 | 1.00 | 14.84 | | C |
| ANISOU | 493 | CG | PHE | A | 69 | 1478 | 2496 | 1663 | −211 | 75 | −717 | C |
| ATOM | 494 | CD1 | PHE | A | 69 | −12.988 | 0.281 | 4.818 | 1.00 | 14.87 | | C |
| ANISOU | 494 | CD1 | PHE | A | 69 | 1226 | 2328 | 2097 | 30 | −85 | −974 | C |
| ATOM | 495 | CD2 | PHE | A | 69 | −12.191 | 2.306 | 3.772 | 1.00 | 15.06 | | C |
| ANISOU | 495 | CD2 | PHE | A | 69 | 1216 | 2717 | 1789 | 273 | −7 | −492 | C |
| ATOM | 496 | CE1 | PHE | A | 69 | −13.611 | −0.092 | 3.634 | 1.00 | 19.18 | | C |
| ANISOU | 496 | CE1 | PHE | A | 69 | 1715 | 3079 | 2493 | −122 | −468 | −1208 | C |
| ATOM | 497 | CE2 | PHE | A | 69 | −12.825 | 1.967 | 2.591 | 1.00 | 18.13 | | C |
| ANISOU | 497 | CE2 | PHE | A | 69 | 1459 | 3936 | 1496 | 115 | 336 | −788 | C |
| ATOM | 498 | CZ | PHE | A | 69 | −13.546 | 0.770 | 2.546 | 1.00 | 19.64 | | C |
| ANISOU | 498 | CZ | PHE | A | 69 | 1623 | 3712 | 2127 | 300 | −371 | −1168 | C |
| ATOM | 499 | N | ARG | A | 70 | −12.310 | 3.095 | 9.545 | 1.00 | 13.76 | | N |
| ANISOU | 499 | N | ARG | A | 70 | 1063 | 2129 | 2035 | −4 | −401 | −858 | N |
| ATOM | 500 | CA | ARG | A | 70 | −11.642 | 3.237 | 10.838 | 1.00 | 13.25 | | C |
| ANISOU | 500 | CA | ARG | A | 70 | 1213 | 2444 | 1380 | −110 | 196 | −456 | C |
| ATOM | 501 | C | ARG | A | 70 | −11.439 | 1.915 | 11.564 | 1.00 | 13.86 | | C |
| ANISOU | 501 | C | ARG | A | 70 | 1137 | 2230 | 1901 | 115 | −75 | −663 | C |
| ATOM | 502 | O | ARG | A | 70 | −10.430 | 1.763 | 12.241 | 1.00 | 14.35 | | O |
| ANISOU | 502 | O | ARG | A | 70 | 1330 | 2446 | 1678 | 164 | −178 | −817 | O |
| ATOM | 503 | CB | ARG | A | 70 | −12.433 | 4.226 | 11.708 | 1.00 | 13.28 | | C |
| ANISOU | 503 | CB | ARG | A | 70 | 1327 | 2322 | 1398 | 239 | −4 | −287 | C |
| ATOM | 504 | CG | ARG | A | 70 | −13.848 | 3.768 | 12.002 | 1.00 | 14.84 | | C |
| ANISOU | 504 | CG | ARG | A | 70 | 1030 | 2943 | 1666 | 395 | −105 | −770 | C |
| ATOM | 505 | CD | ARG | A | 70 | −14.658 | 4.833 | 12.757 | 1.00 | 16.88 | | C |
| ANISOU | 505 | CD | ARG | A | 70 | 1403 | 3107 | 1905 | 710 | −179 | −832 | C |
| ATOM | 506 | NE | ARG | A | 70 | −15.998 | 4.300 | 13.025 | 1.00 | 17.95 | | N |
| ANISOU | 506 | NE | ARG | A | 70 | 1267 | 3566 | 1988 | 1020 | 70 | −632 | N |
| ATOM | 507 | CZ | ARG | A | 70 | −16.921 | 4.948 | 13.757 | 1.00 | 18.40 | | C |
| ANISOU | 507 | CZ | ARG | A | 70 | 1646 | 3249 | 2095 | 1124 | 198 | −434 | C |
| ATOM | 508 | NH1 | ARG | A | 70 | −16.606 | 6.094 | 14.268 | 1.00 | 24.19 | | N |
| ANISOU | 508 | NH1 | ARG | A | 70 | 3026 | 3613 | 2551 | 1815 | −1174 | −1120 | N |
| ATOM | 509 | NH2 | ARG | A | 70 | −18.093 | 4.398 | 13.932 | 1.00 | 24.22 | | N |
| ANISOU | 509 | NH2 | ARG | A | 70 | 1259 | 5473 | 2471 | 1017 | 203 | 39 | N |
| ATOM | 510 | N | SER | A | 71 | −12.342 | 0.942 | 11.425 | 1.00 | 14.20 | | N |
| ANISOU | 510 | N | SER | A | 71 | 1360 | 2302 | 1732 | −23 | −187 | −386 | N |
| ATOM | 511 | CA | SER | A | 71 | −12.184 | −0.317 | 12.116 | 1.00 | 14.72 | | C |
| ANISOU | 511 | CA | SER | A | 71 | 1742 | 2336 | 1515 | 36 | −98 | −420 | C |
| ATOM | 512 | C | SER | A | 71 | −10.979 | −1.087 | 11.641 | 1.00 | 14.10 | | C |
| ANISOU | 512 | C | SER | A | 71 | 1536 | 2193 | 1627 | −58 | −246 | −398 | C |
| ATOM | 513 | O | SER | A | 71 | −10.456 | −1.988 | 12.327 | 1.00 | 15.89 | | O |
| ANISOU | 513 | O | SER | A | 71 | 1461 | 2239 | 2339 | −126 | −239 | −149 | O |
| ATOM | 514 | CB | SER | A | 71 | −13.471 | −1.140 | 11.916 | 1.00 | 21.48 | | C |
| ANISOU | 514 | CB | SER | A | 71 | 1647 | 2942 | 3573 | −405 | 375 | 165 | C |
| ATOM | 515 | OG | SER | A | 71 | −14.507 | −0.559 | 12.723 | 1.00 | 36.29 | | O |
| ANISOU | 515 | OG | SER | A | 71 | 2527 | 5598 | 5663 | −539 | 1758 | −853 | O |
| ATOM | 516 | N | LEU | A | 72 | −10.508 | −0.777 | 10.450 | 1.00 | 14.17 | | N |
| ANISOU | 516 | N | LEU | A | 72 | 1095 | 2426 | 1862 | −280 | −141 | −411 | N |
| ATOM | 517 | CA | LEU | A | 72 | −9.370 | −1.484 | 9.872 | 1.00 | 15.10 | | C |
| ANISOU | 517 | CA | LEU | A | 72 | 1203 | 2651 | 1885 | −91 | −294 | −623 | C |
| ATOM | 518 | C | LEU | A | 72 | −8.067 | −0.806 | 10.194 | 1.00 | 14.20 | | C |
| ANISOU | 518 | C | LEU | A | 72 | 1091 | 2522 | 1781 | 50 | −321 | −501 | C |
| ATOM | 519 | O | LEU | A | 72 | −7.004 | −1.382 | 9.899 | 1.00 | 16.13 | | O |
| ANISOU | 519 | O | LEU | A | 72 | 1145 | 2502 | 2480 | 158 | −373 | −544 | O |
| ATOM | 520 | CB | LEU | A | 72 | −9.571 | −1.564 | 8.364 | 1.00 | 13.99 | | C |
| ANISOU | 520 | CB | LEU | A | 72 | 1235 | 2198 | 1882 | 99 | −418 | −569 | C |
| ATOM | 521 | CG | LEU | A | 72 | −10.780 | −2.356 | 7.869 | 1.00 | 14.93 | | C |
| ANISOU | 521 | CG | LEU | A | 72 | 1140 | 2893 | 1638 | −302 | −149 | −439 | C |
| ATOM | 522 | CD1 | LEU | A | 72 | −10.851 | −2.281 | 6.364 | 1.00 | 18.86 | | C |
| ANISOU | 522 | CD1 | LEU | A | 72 | 1962 | 3580 | 1623 | 180 | −420 | −921 | C |
| ATOM | 523 | CD2 | LEU | A | 72 | −10.671 | −3.809 | 8.323 | 1.00 | 28.71 | | C |
| ANISOU | 523 | CD2 | LEU | A | 72 | 4306 | 2990 | 3613 | −1507 | −1334 | 211 | C |
| ATOM | 524 | N | ARG | A | 73 | −8.123 | 0.381 | 10.797 | 1.00 | 14.81 | | N |
| ANISOU | 524 | N | ARG | A | 73 | 942 | 2507 | 2178 | −147 | 20 | −560 | N |
| ATOM | 525 | CA | ARG | A | 73 | −6.909 | 1.103 | 11.115 | 1.00 | 14.76 | | C |
| ANISOU | 525 | CA | ARG | A | 73 | 933 | 2526 | 2149 | −355 | 66 | −183 | C |
| ATOM | 526 | C | ARG | A | 73 | −6.519 | 0.809 | 12.565 | 1.00 | 15.07 | | C |
| ANISOU | 526 | C | ARG | A | 73 | 1003 | 2790 | 1932 | 218 | 22 | −730 | C |
| ATOM | 527 | O | ARG | A | 73 | −5.332 | 0.766 | 12.894 | 1.00 | 15.42 | | O |
| ANISOU | 527 | O | ARG | A | 73 | 1010 | 2139 | 2709 | 279 | −232 | −958 | O |
| ATOM | 528 | CB | ARG | A | 73 | −7.057 | 2.617 | 10.978 | 1.00 | 18.61 | | C |
| ANISOU | 528 | CB | ARG | A | 73 | 2551 | 2435 | 2087 | −548 | 625 | −356 | C |
| ATOM | 529 | CG | ARG | A | 73 | −7.813 | 3.236 | 12.150 | 1.00 | 24.71 | | C |
| ANISOU | 529 | CG | ARG | A | 73 | 2935 | 3689 | 2764 | 117 | 446 | −1225 | C |
| ATOM | 530 | CD | ARG | A | 73 | −7.893 | 4.705 | 12.178 | 1.00 | 24.64 | | C |
| ANISOU | 530 | CD | ARG | A | 73 | 3041 | 3804 | 2515 | 1001 | −471 | −1140 | C |
| ATOM | 531 | NE | ARG | A | 73 | −8.639 | 5.380 | 13.230 | 1.00 | 22.12 | | N |
| ANISOU | 531 | NE | ARG | A | 73 | 2373 | 3676 | 2353 | 1255 | −442 | −448 | N |
| ATOM | 532 | CZ | ARG | A | 73 | −9.416 | 6.443 | 13.095 | 1.00 | 21.12 | | C |
| ANISOU | 532 | CZ | ARG | A | 73 | 2534 | 3445 | 2045 | 1060 | 270 | 368 | C |
| ATOM | 533 | NH1 | ARG | A | 73 | −9.657 | 7.056 | 11.951 | 1.00 | 24.31 | | N |
| ANISOU | 533 | NH1 | ARG | A | 73 | 3776 | 3170 | 2293 | 316 | 656 | 939 | N |
| ATOM | 534 | NH2 | ARG | A | 73 | −10.030 | 6.954 | 14.166 | 1.00 | 17.04 | | N |
| ANISOU | 534 | NH2 | ARG | A | 73 | 1994 | 2612 | 1870 | 405 | −426 | −344 | N |
| ATOM | 535 | N | THR | A | 74 | −7.450 | 0.604 | 13.481 | 1.00 | 15.63 | | N |
| ANISOU | 535 | N | THR | A | 74 | 1178 | 3039 | 1723 | −4 | −83 | −969 | N |
| ATOM | 536 | CA | THR | A | 74 | −7.104 | 0.413 | 14.897 | 1.00 | 15.26 | | C |
| ANISOU | 536 | CA | THR | A | 74 | 1225 | 2831 | 1742 | −161 | 38 | −959 | C |
| ATOM | 537 | C | THR | A | 74 | −6.285 | −0.837 | 15.146 | 1.00 | 15.38 | | C |
| ANISOU | 537 | C | THR | A | 74 | 1501 | 2763 | 1578 | −128 | 99 | −811 | C |
| ATOM | 538 | O | THR | A | 74 | −5.516 | −0.798 | 16.153 | 1.00 | 16.28 | | O |
| ANISOU | 538 | O | THR | A | 74 | 1461 | 2655 | 2072 | −268 | −179 | −864 | O |
| ATOM | 539 | CB | THR | A | 74 | −8.398 | 0.423 | 15.716 | 1.00 | 16.64 | | C |
| ANISOU | 539 | CB | THR | A | 74 | 1137 | 3295 | 1890 | −263 | 34 | −876 | C |
| ATOM | 540 | OG1 | THR | A | 74 | −9.279 | −0.628 | 15.306 | 1.00 | 21.00 | | O |
| ANISOU | 540 | OG1 | THR | A | 74 | 1673 | 3875 | 2432 | −826 | 602 | −1515 | O |
| ATOM | 541 | CG2 | THR | A | 74 | −9.185 | 1.702 | 15.576 | 1.00 | 20.10 | | C |
| ANISOU | 541 | CG2 | THR | A | 74 | 1529 | 3683 | 2427 | 223 | 261 | −1105 | C |
| ATOM | 542 | N | PRO | A | 75 | −6.296 | −1.946 | 14.396 | 1.00 | 17.24 | | N |
| ANISOU | 542 | N | PRO | A | 75 | 1717 | 2929 | 1903 | 128 | −290 | −1014 | N |
| ATOM | 543 | CA | PRO | A | 75 | −5.330 | −3.024 | 14.677 | 1.00 | 16.94 | | C |
| ANISOU | 543 | CA | PRO | A | 75 | 1641 | 2379 | 2418 | −292 | −220 | −673 | C |
| ATOM | 544 | C | PRO | A | 75 | −3.880 | −2.542 | 14.700 | 1.00 | 18.08 | | C |
| ANISOU | 544 | C | PRO | A | 75 | 1651 | 2604 | 2616 | −397 | −643 | −292 | C |
| ATOM | 545 | O | PRO | A | 75 | −3.013 | −3.159 | 15.337 | 1.00 | 17.58 | | O |
| ANISOU | 545 | O | PRO | A | 75 | 1604 | 2894 | 2183 | 105 | −9 | −295 | O |
| ATOM | 546 | CB | PRO | A | 75 | −5.547 | −4.027 | 13.537 | 1.00 | 19.63 | | C |
| ANISOU | 546 | CB | PRO | A | 75 | 1986 | 2306 | 3169 | −197 | −593 | −993 | C |
| ATOM | 547 | CG | PRO | A | 75 | −6.997 | −3.865 | 13.230 | 1.00 | 18.89 | | C |
| ANISOU | 547 | CG | PRO | A | 75 | 1996 | 2545 | 2636 | −380 | −567 | −482 | C |
| ATOM | 548 | CD | PRO | A | 75 | −7.203 | −2.355 | 13.319 | 1.00 | 16.09 | | C |
| ANISOU | 548 | CD | PRO | A | 75 | 1441 | 2672 | 2001 | −259 | −66 | −865 | C |
| ATOM | 549 | N | PHE | A | 76 | −3.594 | −1.450 | 13.988 | 1.00 | 14.53 | | N |
| ANISOU | 549 | N | PHE | A | 76 | 1274 | 2102 | 2144 | 40 | −43 | −818 | N |
| ATOM | 550 | CA | PHE | A | 76 | −2.228 | −0.941 | 13.838 | 1.00 | 14.83 | | C |
| ANISOU | 550 | CA | PHE | A | 76 | 1133 | 2320 | 2182 | 126 | −121 | −781 | C |
| ATOM | 551 | C | PHE | A | 76 | −1.822 | −0.050 | 14.995 | 1.00 | 14.61 | | C |
| ANISOU | 551 | C | PHE | A | 76 | 969 | 2512 | 2069 | 214 | −225 | −804 | C |
| ATOM | 552 | O | PHE | A | 76 | −0.649 | 0.366 | 15.055 | 1.00 | 15.67 | | O |
| ANISOU | 552 | O | PHE | A | 76 | 885 | 3135 | 1933 | 223 | −162 | −916 | O |
| ATOM | 553 | CB | PHE | A | 76 | −2.066 | −0.211 | 12.504 | 1.00 | 15.95 | | C |
| ANISOU | 553 | CB | PHE | A | 76 | 1070 | 2963 | 2026 | −27 | −16 | −743 | C |
| ATOM | 554 | CG | PHE | A | 76 | −2.195 | −1.259 | 11.405 | 1.00 | 16.20 | | C |
| ANISOU | 554 | CG | PHE | A | 76 | 1211 | 2754 | 2191 | −479 | 385 | −729 | C |
| ATOM | 555 | CD1 | PHE | A | 76 | −3.435 | −1.398 | 10.804 | 1.00 | 18.45 | | C |
| ANISOU | 555 | CD1 | PHE | A | 76 | 1450 | 3520 | 2040 | −109 | 170 | −1273 | C |
| ATOM | 556 | CD2 | PHE | A | 76 | −1.165 | −2.105 | 11.023 | 1.00 | 18.44 | | C |
| ANISOU | 556 | CD2 | PHE | A | 76 | 1204 | 3608 | 2195 | −414 | 441 | −1300 | C |
| ATOM | 557 | CE1 | PHE | A | 76 | −3.617 | −2.373 | 9.854 | 1.00 | 17.69 | | C |
| ANISOU | 557 | CE1 | PHE | A | 76 | 1861 | 2674 | 2186 | −489 | 252 | −892 | C |
| ATOM | 558 | CE2 | PHE | A | 76 | −1.330 | −3.073 | 10.065 | 1.00 | 15.99 | | C |
| ANISOU | 558 | CE2 | PHE | A | 76 | 1854 | 2702 | 1520 | −493 | 417 | −515 | C |
| ATOM | 559 | CZ | PHE | A | 76 | −2.585 | −3.202 | 9.515 | 1.00 | 15.79 | | C |
| ANISOU | 559 | CZ | PHE | A | 76 | 1959 | 2463 | 1579 | −482 | 283 | −616 | C |
| ATOM | 560 | N | TYR | A | 77 | −2.730 | 0.223 | 15.905 | 1.00 | 12.95 | | N |
| ANISOU | 560 | N | TYR | A | 77 | 745 | 1983 | 2191 | 183 | −291 | −774 | N |
| ATOM | 561 | CA | TYR | A | 77 | −2.348 | 0.885 | 17.169 | 1.00 | 11.92 | | C |
| ANISOU | 561 | CA | TYR | A | 77 | 1016 | 1769 | 1744 | 1 | −99 | −337 | C |
| ATOM | 562 | C | TYR | A | 77 | −1.448 | 0.011 | 18.028 | 1.00 | 12.97 | | C |
| ANISOU | 562 | C | TYR | A | 77 | 857 | 1809 | 2261 | −262 | −324 | −52 | C |
| ATOM | 563 | O | TYR | A | 77 | −0.571 | 0.509 | 18.722 | 1.00 | 14.51 | | O |
| ANISOU | 563 | O | TYR | A | 77 | 1131 | 2249 | 2135 | 11 | −436 | −638 | O |
| ATOM | 564 | CB | TYR | A | 77 | −3.572 | 1.255 | 17.999 | 1.00 | 12.37 | | C |
| ANISOU | 564 | CB | TYR | A | 77 | 1070 | 1900 | 1728 | −59 | 100 | −140 | C |
| ATOM | 565 | CG | TYR | A | 77 | −4.418 | 2.347 | 17.368 | 1.00 | 13.66 | | C |
| ANISOU | 565 | CG | TYR | A | 77 | 1350 | 2366 | 1472 | 356 | 0 | −283 | C |
| ATOM | 566 | CD1 | TYR | A | 77 | −4.263 | 2.870 | 16.102 | 1.00 | 14.92 | | C |
| ANISOU | 566 | CD1 | TYR | A | 77 | 1477 | 2463 | 1727 | 419 | 122 | −17 | C |
| ATOM | 567 | CD2 | TYR | A | 77 | −5.439 | 2.885 | 18.114 | 1.00 | 13.62 | | C |
| ANISOU | 567 | CD2 | TYR | A | 77 | 1138 | 2629 | 1407 | 370 | −247 | −418 | C |
| ATOM | 568 | CE1 | TYR | A | 77 | −5.100 | 3.873 | 15.623 | 1.00 | 15.57 | | C |
| ANISOU | 568 | CE1 | TYR | A | 77 | 1532 | 2387 | 1998 | 390 | 349 | 137 | C |
| ATOM | 569 | CE2 | TYR | A | 77 | −6.272 | 3.862 | 17.634 | 1.00 | 14.33 | | C |
| ANISOU | 569 | CE2 | TYR | A | 77 | 1130 | 2714 | 1601 | 389 | −86 | −200 | C |
| ATOM | 570 | CZ | TYR | A | 77 | −6.132 | 4.371 | 16.385 | 1.00 | 12.88 | | C |
| ANISOU | 570 | CZ | TYR | A | 77 | 999 | 2187 | 1708 | 87 | −82 | −211 | C |
| ATOM | 571 | OH | TYR | A | 77 | −6.938 | 5.364 | 15.899 | 1.00 | 16.43 | | O |
| ANISOU | 571 | OH | TYR | A | 77 | 1864 | 2399 | 1981 | 601 | −94 | −159 | O |
| ATOM | 572 | N | ARG | A | 78 | −1.715 | −1.282 | 18.057 | 1.00 | 19.12 | | N |
| ANISOU | 572 | N | ARG | A | 78 | 1526 | 1637 | 4102 | 183 | −1090 | −354 | N |
| ATOM | 573 | CA | ARG | A | 78 | −1.049 | −2.223 | 18.962 | 1.00 | 20.03 | | C |
| ANISOU | 573 | CA | ARG | A | 78 | 1456 | 1709 | 4445 | 82 | −757 | 115 | C |
| ATOM | 574 | C | ARG | A | 78 | 0.444 | −2.212 | 18.656 | 1.00 | 18.89 | | C |
| ANISOU | 574 | C | ARG | A | 78 | 1512 | 2413 | 3253 | 332 | −724 | 89 | C |
| ATOM | 575 | O | ARG | A | 78 | 0.896 | −2.114 | 17.516 | 1.00 | 17.71 | | O |
| ANISOU | 575 | O | ARG | A | 78 | 2001 | 1774 | 2953 | 280 | −900 | −390 | O |
| ATOM | 576 | CB | ARG | A | 78 | −1.721 | −3.590 | 18.845 | 1.00 | 30.93 | | C |
| ANISOU | 576 | CB | ARG | A | 78 | 3467 | 2465 | 5820 | −1281 | −2152 | 1230 | C |
| ATOM | 577 | CG | ARG | A | 78 | −1.094 | −4.748 | 19.616 | 1.00 | 44.62 | | C |
| ANISOU | 577 | CG | ARG | A | 78 | 7142 | 2143 | 7668 | −1237 | −4075 | 1315 | C |
| ATOM | 578 | CD | ARG | A | 78 | −1.115 | −6.019 | 18.747 | 1.00 | 52.31 | | C |
| ANISOU | 578 | CD | ARG | A | 78 | 9270 | 2656 | 7951 | −129 | −5877 | 859 | C |
| ATOM | 579 | NE | ARG | A | 78 | −2.292 | −5.992 | 17.901 | 1.00 | 65.93 | | N |
| ANISOU | 579 | NE | ARG | A | 78 | 10637 | 6859 | 7555 | −485 | −6668 | 448 | N |
| ATOM | 580 | CZ | ARG | A | 78 | −2.431 | −5.872 | 16.585 | 1.00 | 68.97 | | C |
| ANISOU | 580 | CZ | ARG | A | 78 | 11262 | 7634 | 7310 | −1312 | −6126 | −68 | C |
| ATOM | 581 | NH1 | ARG | A | 78 | −1.389 | −5.747 | 15.774 | 1.00 | 79.85 | | N |
| ANISOU | 581 | NH1 | ARG | A | 78 | 11940 | 10231 | 8168 | −682 | −5024 | −1936 | N |
| ATOM | 582 | NH2 | ARG | A | 78 | −3.645 | −5.870 | 16.063 | 1.00 | 59.94 | | N |
| ANISOU | 582 | NH2 | ARG | A | 78 | 11818 | 4183 | 6773 | −2834 | −6651 | 1488 | N |
| ATOM | 583 | N | GLY | A | 79 | 1.231 | −2.290 | 19.731 | 1.00 | 18.19 | | N |
| ANISOU | 583 | N | GLY | A | 79 | 1479 | 2498 | 2933 | −312 | −505 | 394 | N |
| ATOM | 584 | CA | GLY | A | 79 | 2.663 | −2.316 | 19.615 | 1.00 | 16.69 | | C |
| ANISOU | 584 | CA | GLY | A | 79 | 1454 | 1752 | 3136 | −5 | −656 | −557 | C |
| ATOM | 585 | C | GLY | A | 79 | 3.284 | −0.947 | 19.554 | 1.00 | 14.03 | | C |
| ANISOU | 585 | C | GLY | A | 79 | 1127 | 1727 | 2476 | 142 | −350 | −194 | C |
| ATOM | 586 | O | GLY | A | 79 | 4.504 | −0.816 | 19.538 | 1.00 | 14.78 | | O |
| ANISOU | 586 | O | GLY | A | 79 | 997 | 2233 | 2384 | 143 | −141 | −429 | O |
| ATOM | 587 | N | SER | A | 80 | 2.465 | 0.091 | 19.562 | 1.00 | 12.57 | | N |
| ANISOU | 587 | N | SER | A | 80 | 1112 | 1785 | 1878 | 166 | −211 | −428 | N |
| ATOM | 588 | CA | SER | A | 80 | 2.977 | 1.450 | 19.687 | 1.00 | 11.05 | | C |
| ANISOU | 588 | CA | SER | A | 80 | 1022 | 1727 | 1450 | 29 | −6 | −125 | C |
| ATOM | 589 | C | SER | A | 80 | 3.648 | 1.647 | 21.045 | 1.00 | 11.82 | | C |
| ANISOU | 589 | C | SER | A | 80 | 1162 | 1817 | 1512 | −159 | −71 | −115 | C |
| ATOM | 590 | O | SER | A | 80 | 3.143 | 1.223 | 22.074 | 1.00 | 15.57 | | O |
| ANISOU | 590 | O | SER | A | 80 | 1667 | 2637 | 1612 | −707 | −131 | 264 | O |
| ATOM | 591 | CB | SER | A | 80 | 1.864 | 2.473 | 19.545 | 1.00 | 14.34 | | C |
| ANISOU | 591 | CB | SER | A | 80 | 1698 | 1878 | 1872 | 445 | −549 | −757 | C |
| ATOM | 592 | OG | SER | A | 80 | 1.221 | 2.374 | 18.267 | 1.00 | 15.02 | | O |
| ANISOU | 592 | OG | SER | A | 80 | 1793 | 1986 | 1930 | 505 | −692 | −688 | O |
| ATOM | 593 | N | ASP | A | 81 | 4.799 | 2.291 | 21.018 | 1.00 | 10.95 | | N |
| ANISOU | 593 | N | ASP | A | 81 | 1040 | 1708 | 1411 | −43 | 8 | −127 | N |
| ATOM | 594 | CA | ASP | A | 81 | 5.556 | 2.559 | 22.249 | 1.00 | 10.97 | | C |
| ANISOU | 594 | CA | ASP | A | 81 | 1163 | 1469 | 1535 | 4 | −195 | −54 | C |
| ATOM | 595 | C | ASP | A | 81 | 5.287 | 3.936 | 22.809 | 1.00 | 10.63 | | C |
| ANISOU | 595 | C | ASP | A | 81 | 1033 | 1540 | 1464 | 74 | −223 | −52 | C |
| ATOM | 596 | O | ASP | A | 81 | 5.520 | 4.156 | 23.999 | 1.00 | 11.21 | | O |
| ANISOU | 596 | O | ASP | A | 81 | 1083 | 1715 | 1461 | 1 | −160 | −110 | O |
| ATOM | 597 | CB | ASP | A | 81 | 7.055 | 2.406 | 21.937 | 1.00 | 12.50 | | C |
| ANISOU | 597 | CB | ASP | A | 81 | 1157 | 1510 | 2084 | 104 | −204 | −241 | C |
| ATOM | 598 | CG | ASP | A | 81 | 7.438 | 1.035 | 21.456 | 1.00 | 13.83 | | C |
| ANISOU | 598 | CG | ASP | A | 81 | 1837 | 1513 | 1904 | 279 | 103 | −57 | C |
| ATOM | 599 | OD1 | ASP | A | 81 | 7.156 | 0.040 | 22.156 | 1.00 | 18.02 | | O |
| ANISOU | 599 | OD1 | ASP | A | 81 | 2769 | 1650 | 2427 | 286 | 466 | 222 | O |
| ATOM | 600 | OD2 | ASP | A | 81 | 8.063 | 0.858 | 20.371 | 1.00 | 15.95 | | O |
| ANISOU | 600 | OD2 | ASP | A | 81 | 2194 | 1981 | 1884 | 456 | 142 | −90 | O |
| ATOM | 601 | N | CYS | A | 82 | 4.862 | 4.899 | 22.000 | 1.00 | 10.82 | | N |
| ANISOU | 601 | N | CYS | A | 82 | 904 | 1621 | 1585 | 211 | −141 | −37 | N |
| ATOM | 602 | CA | CYS | A | 82 | 4.533 | 6.232 | 22.420 | 1.00 | 9.88 | | C |
| ANISOU | 602 | CA | CYS | A | 82 | 863 | 1615 | 1277 | 202 | −5 | −46 | C |
| ATOM | 603 | C | CYS | A | 82 | 3.454 | 6.774 | 21.476 | 1.00 | 8.04 | | C |
| ANISOU | 603 | C | CYS | A | 82 | 687 | 1279 | 1087 | 28 | 75 | −247 | C |
| ATOM | 604 | O | CYS | A | 82 | 3.467 | 6.407 | 20.321 | 1.00 | 10.85 | | O |
| ANISOU | 604 | O | CYS | A | 82 | 904 | 2058 | 1162 | 252 | 51 | −531 | O |
| ATOM | 605 | CB | CYS | A | 82 | 5.756 | 7.128 | 22.404 | 1.00 | 12.32 | | C |
| ANISOU | 605 | CB | CYS | A | 82 | 688 | 1949 | 2045 | 182 | −131 | −536 | C |
| ATOM | 606 | SG | CYS | A | 82 | 5.546 | 8.844 | 22.949 | 1.00 | 14.09 | | S |
| ANISOU | 606 | SG | CYS | A | 82 | 1374 | 1763 | 2217 | −87 | −16 | −246 | S |
| ATOM | 607 | N | CYS | A | 83 | 2.598 | 7.627 | 21.966 | 1.00 | 9.87 | | N |
| ANISOU | 607 | N | CYS | A | 83 | 1031 | 1640 | 1079 | 394 | 14 | −344 | N |
| ATOM | 608 | CA | CYS | A | 83 | 1.539 | 8.211 | 21.144 | 1.00 | 10.60 | | C |
| ANISOU | 608 | CA | CYS | A | 83 | 1052 | 1506 | 1469 | 306 | −87 | −286 | C |
| ATOM | 609 | C | CYS | A | 83 | 1.686 | 9.713 | 21.191 | 1.00 | 9.74 | | C |
| ANISOU | 609 | C | CYS | A | 83 | 957 | 1561 | 1182 | 152 | −593 | −351 | C |
| ATOM | 610 | O | CYS | A | 83 | 1.743 | 10.291 | 22.303 | 1.00 | 10.14 | | O |
| ANISOU | 610 | O | CYS | A | 83 | 1411 | 1482 | 961 | 139 | −314 | −103 | O |
| ATOM | 611 | CB | CYS | A | 83 | 0.170 | 7.822 | 21.696 | 1.00 | 13.23 | | C |
| ANISOU | 611 | CB | CYS | A | 83 | 944 | 1814 | 2267 | 36 | −192 | −495 | C |
| ATOM | 612 | SG | CYS | A | 83 | −1.217 | 8.571 | 20.942 | 1.00 | 15.82 | | S |
| ANISOU | 612 | SG | CYS | A | 83 | 1138 | 2412 | 2462 | 254 | −351 | −577 | S |
| ATOM | 613 | N | LEU | A | 84 | 1.746 | 10.370 | 20.068 | 1.00 | 9.34 | | N |
| ANISOU | 613 | N | LEU | A | 84 | 1094 | 1451 | 1004 | 292 | −301 | −514 | N |
| ATOM | 614 | CA | LEU | A | 84 | 1.824 | 11.819 | 19.931 | 1.00 | 10.33 | | C |
| ANISOU | 614 | CA | LEU | A | 84 | 1043 | 1448 | 1436 | 149 | −424 | −385 | C |
| ATOM | 615 | C | LEU | A | 84 | 0.445 | 12.346 | 19.554 | 1.00 | 9.00 | | C |
| ANISOU | 615 | C | LEU | A | 84 | 1116 | 1277 | 1028 | 165 | −409 | −354 | C |
| ATOM | 616 | O | LEU | A | 84 | −0.044 | 12.155 | 18.441 | 1.00 | 13.66 | | O |
| ANISOU | 616 | O | LEU | A | 84 | 1271 | 2596 | 1321 | 269 | −531 | −1017 | O |
| ATOM | 617 | CB | LEU | A | 84 | 2.857 | 12.195 | 18.896 | 1.00 | 12.41 | | C |
| ANISOU | 617 | CB | LEU | A | 84 | 1162 | 1698 | 1856 | −67 | −153 | −467 | C |
| ATOM | 618 | CG | LEU | A | 84 | 4.331 | 11.713 | 18.967 | 1.00 | 22.23 | | C |
| ANISOU | 618 | CG | LEU | A | 84 | 772 | 4107 | 3568 | −285 | −381 | 686 | C |
| ATOM | 619 | CD1 | LEU | A | 84 | 5.253 | 12.883 | 18.703 | 1.00 | 21.51 | | C |
| ANISOU | 619 | CD1 | LEU | A | 84 | 1059 | 2733 | 4382 | 281 | 303 | −298 | C |
| ATOM | 620 | CD2 | LEU | A | 84 | 4.679 | 10.913 | 20.170 | 1.00 | 30.13 | | C |
| ANISOU | 620 | CD2 | LEU | A | 84 | 2407 | 3972 | 5070 | 265 | −1291 | 1313 | C |
| ATOM | 621 | N | LEU | A | 85 | −0.192 | 12.989 | 20.492 | 1.00 | 8.71 | | N |
| ANISOU | 621 | N | LEU | A | 85 | 1060 | 1202 | 1048 | 88 | −316 | −319 | N |
| ATOM | 622 | CA | LEU | A | 85 | −1.513 | 13.543 | 20.308 | 1.00 | 8.37 | | C |
| ANISOU | 622 | CA | LEU | A | 85 | 1001 | 1006 | 1173 | −40 | −372 | −272 | C |
| ATOM | 623 | C | LEU | A | 85 | −1.345 | 14.978 | 19.781 | 1.00 | 8.97 | | C |
| ANISOU | 623 | C | LEU | A | 85 | 1116 | 1080 | 1214 | −115 | −519 | −158 | C |
| ATOM | 624 | O | LEU | A | 85 | −0.623 | 15.793 | 20.434 | 1.00 | 9.16 | | O |
| ANISOU | 624 | O | LEU | A | 85 | 970 | 1046 | 1464 | −99 | −552 | −273 | O |
| ATOM | 625 | CB | LEU | A | 85 | −2.310 | 13.538 | 21.614 | 1.00 | 11.08 | | C |
| ANISOU | 625 | CB | LEU | A | 85 | 886 | 1762 | 1561 | −534 | −30 | −266 | C |
| ATOM | 626 | CG | LEU | A | 85 | −2.539 | 12.215 | 22.322 | 1.00 | 10.76 | | C |
| ANISOU | 626 | CG | LEU | A | 85 | 976 | 1727 | 1384 | −333 | 66 | −383 | C |
| ATOM | 627 | CD1 | LEU | A | 85 | −3.171 | 12.415 | 23.677 | 1.00 | 16.39 | | C |
| ANISOU | 627 | CD1 | LEU | A | 85 | 2631 | 1631 | 1965 | −287 | 1150 | −446 | C |
| ATOM | 628 | CD2 | LEU | A | 85 | −3.497 | 11.361 | 21.509 | 1.00 | 14.98 | | C |
| ANISOU | 628 | CD2 | LEU | A | 85 | 1608 | 1477 | 2607 | −372 | −697 | −484 | C |
| ATOM | 629 | N | THR | A | 86 | −1.889 | 15.258 | 18.653 | 1.00 | 8.01 | | N |
| ANISOU | 629 | N | THR | A | 86 | 880 | 1009 | 1156 | 64 | −390 | −293 | N |
| ATOM | 630 | CA | THR | A | 86 | −1.671 | 16.488 | 17.929 | 1.00 | 8.06 | | C |
| ANISOU | 630 | CA | THR | A | 86 | 744 | 1264 | 1056 | 120 | −211 | −155 | C |
| ATOM | 631 | C | THR | A | 86 | −2.924 | 17.318 | 17.755 | 1.00 | 7.31 | | C |
| ANISOU | 631 | C | THR | A | 86 | 607 | 966 | 1203 | −63 | −35 | 72 | C |
| ATOM | 632 | O | THR | A | 86 | −3.973 | 16.753 | 17.403 | 1.00 | 9.06 | | O |
| ANISOU | 632 | O | THR | A | 86 | 638 | 1409 | 1394 | −25 | −98 | −438 | O |
| ATOM | 633 | CB | THR | A | 86 | −1.107 | 16.119 | 16.531 | 1.00 | 11.11 | | C |
| ANISOU | 633 | CB | THR | A | 86 | 1140 | 1853 | 1229 | 221 | 36 | −315 | C |
| ATOM | 634 | OG1 | THR | A | 86 | −0.002 | 15.235 | 16.685 | 1.00 | 13.38 | | O |
| ANISOU | 634 | OG1 | THR | A | 86 | 1396 | 2303 | 1383 | 559 | 26 | −417 | O |
| ATOM | 635 | CG2 | THR | A | 86 | −0.610 | 17.346 | 15.807 | 1.00 | 11.82 | | C |
| ANISOU | 635 | CG2 | THR | A | 86 | 952 | 2416 | 1125 | −190 | −147 | −24 | C |
| ATOM | 636 | N | PHE | A | 87 | −2.804 | 18.605 | 18.005 | 1.00 | 8.35 | | N |
| ANISOU | 636 | N | PHE | A | 87 | 593 | 1102 | 1477 | −6 | −173 | −114 | N |
| ATOM | 637 | CA | PHE | A | 87 | −3.876 | 19.535 | 17.689 | 1.00 | 7.55 | | C |
| ANISOU | 637 | CA | PHE | A | 87 | 989 | 983 | 896 | 151 | −151 | −67 | C |
| ATOM | 638 | C | PHE | A | 87 | −3.254 | 20.713 | 16.963 | 1.00 | 7.56 | | C |
| ANISOU | 638 | C | PHE | A | 87 | 703 | 1279 | 890 | 129 | −126 | −72 | C |
| ATOM | 639 | O | PHE | A | 87 | −2.030 | 20.733 | 16.839 | 1.00 | 9.25 | | O |
| ANISOU | 639 | O | PHE | A | 87 | 697 | 1560 | 1255 | 64 | −134 | −43 | O |
| ATOM | 640 | CB | PHE | A | 87 | −4.649 | 19.968 | 18.932 | 1.00 | 7.73 | | C |
| ANISOU | 640 | CB | PHE | A | 87 | 730 | 1305 | 902 | 29 | −153 | −153 | C |
| ATOM | 641 | CG | PHE | A | 87 | −3.878 | 20.880 | 19.872 | 1.00 | 7.50 | | C |
| ANISOU | 641 | CG | PHE | A | 87 | 633 | 1212 | 1006 | 2 | −117 | −195 | C |
| ATOM | 642 | CD1 | PHE | A | 87 | −3.011 | 20.310 | 20.797 | 1.00 | 7.85 | | C |
| ANISOU | 642 | CD1 | PHE | A | 87 | 772 | 1348 | 862 | 174 | −114 | −337 | C |
| ATOM | 643 | CD2 | PHE | A | 87 | −4.000 | 22.236 | 19.818 | 1.00 | 8.17 | | C |
| ANISOU | 643 | CD2 | PHE | A | 87 | 748 | 1264 | 1090 | 103 | −78 | −254 | C |
| ATOM | 644 | CE1 | PHE | A | 87 | −2.299 | 21.125 | 21.664 | 1.00 | 8.23 | | C |
| ANISOU | 644 | CE1 | PHE | A | 87 | 775 | 1464 | 889 | 337 | −101 | −472 | C |
| ATOM | 645 | CE2 | PHE | A | 87 | −3.302 | 23.072 | 20.684 | 1.00 | 8.57 | | C |
| ANISOU | 645 | CE2 | PHE | A | 87 | 920 | 1181 | 1154 | −89 | −80 | −140 | C |
| ATOM | 646 | CZ | PHE | A | 87 | −2.439 | 22.487 | 21.599 | 1.00 | 9.26 | | C |
| ANISOU | 646 | CZ | PHE | A | 87 | 772 | 1407 | 1341 | 1 | −200 | −319 | C |
| ATOM | 647 | N | SER | A | 88 | −4.030 | 21.637 | 16.447 | 1.00 | 7.66 | | N |
| ANISOU | 647 | N | SER | A | 88 | 795 | 1206 | 910 | 136 | −7 | 145 | N |
| ATOM | 648 | CA | SER | A | 88 | −3.560 | 22.844 | 15.804 | 1.00 | 9.74 | | C |
| ANISOU | 648 | CA | SER | A | 88 | 1019 | 1036 | 1645 | 52 | 246 | −52 | C |
| ATOM | 649 | C | SER | A | 88 | −3.934 | 24.039 | 16.663 | 1.00 | 8.69 | | C |
| ANISOU | 649 | C | SER | A | 88 | 899 | 1238 | 1163 | 201 | −67 | −108 | C |
| ATOM | 650 | O | SER | A | 88 | −5.098 | 24.154 | 17.114 | 1.00 | 9.68 | | O |
| ANISOU | 650 | O | SER | A | 88 | 971 | 1467 | 1242 | 250 | −19 | −22 | O |
| ATOM | 651 | CB | SER | A | 88 | −4.088 | 22.996 | 14.326 | 1.00 | 19.51 | | C |
| ANISOU | 651 | CB | SER | A | 88 | 5286 | 1525 | 601 | 263 | 1011 | 203 | C |
| ATOM | 652 | OG | SER | A | 88 | −3.438 | 24.160 | 13.849 | 1.00 | 42.36 | | O |
| ANISOU | 652 | OG | SER | A | 88 | 9343 | 3957 | 2796 | −3077 | −1087 | 1599 | O |
| ATOM | 653 | N | VAL | A | 89 | −3.032 | 24.971 | 16.916 | 1.00 | 9.85 | | N |
| ANISOU | 653 | N | VAL | A | 89 | 1246 | 1296 | 1201 | −1 | −42 | −96 | N |
| ATOM | 654 | CA | VAL | A | 89 | −3.307 | 26.077 | 17.805 | 1.00 | 9.23 | | C |
| ANISOU | 654 | CA | VAL | A | 89 | 1009 | 1236 | 1261 | 102 | −55 | −82 | C |
| ATOM | 655 | C | VAL | A | 89 | −4.298 | 27.078 | 17.230 | 1.00 | 10.81 | | C |
| ANISOU | 655 | C | VAL | A | 89 | 1178 | 1639 | 1292 | 313 | −4 | 31 | C |
| ATOM | 656 | O | VAL | A | 89 | −4.816 | 27.893 | 17.996 | 1.00 | 11.13 | | O |
| ANISOU | 656 | O | VAL | A | 89 | 1397 | 1224 | 1608 | 251 | −1 | −12 | O |
| ATOM | 657 | CB | VAL | A | 89 | −2.021 | 26.816 | 18.252 | 1.00 | 10.54 | | C |
| ANISOU | 657 | CB | VAL | A | 89 | 1059 | 1460 | 1484 | 27 | −35 | −280 | C |
| ATOM | 658 | CG1 | VAL | A | 89 | −1.100 | 25.837 | 18.938 | 1.00 | 11.22 | | C |
| ANISOU | 658 | CG1 | VAL | A | 89 | 1103 | 1657 | 1502 | 31 | −316 | −421 | C |
| ATOM | 659 | CG2 | VAL | A | 89 | −1.298 | 27.505 | 17.127 | 1.00 | 12.60 | | C |
| ANISOU | 659 | CG2 | VAL | A | 89 | 1298 | 1359 | 2130 | −121 | 268 | −151 | C |
| ATOM | 660 | N | ASP | A | 90 | −4.606 | 27.006 | 15.955 | 1.00 | 11.65 | | N |
| ANISOU | 660 | N | ASP | A | 90 | 1179 | 1805 | 1442 | 320 | −194 | 32 | N |
| ATOM | 661 | CA | ASP | A | 90 | −5.636 | 27.808 | 15.322 | 1.00 | 13.06 | | C |
| ANISOU | 661 | CA | ASP | A | 90 | 1383 | 1728 | 1853 | 144 | −427 | 330 | C |
| ATOM | 662 | C | ASP | A | 90 | −7.006 | 27.147 | 15.344 | 1.00 | 15.36 | | C |
| ANISOU | 662 | C | ASP | A | 90 | 1218 | 2167 | 2450 | 204 | −416 | 708 | C |
| ATOM | 663 | O | ASP | A | 90 | −7.887 | 27.715 | 14.700 | 1.00 | 19.40 | | O |
| ANISOU | 663 | O | ASP | A | 90 | 1362 | 2988 | 3022 | 161 | −560 | 1396 | O |
| ATOM | 664 | CB | ASP | A | 90 | −5.244 | 28.146 | 13.889 | 1.00 | 16.04 | | C |
| ANISOU | 664 | CB | ASP | A | 90 | 1885 | 2251 | 1959 | −172 | −496 | 719 | C |
| ATOM | 665 | CG | ASP | A | 90 | −5.297 | 26.936 | 12.989 | 1.00 | 19.92 | | C |
| ANISOU | 665 | CG | ASP | A | 90 | 2157 | 3241 | 2169 | −423 | 591 | −83 | C |
| ATOM | 666 | OD1 | ASP | A | 90 | −4.972 | 25.854 | 13.500 | 1.00 | 25.38 | | O |
| ANISOU | 666 | OD1 | ASP | A | 90 | 4839 | 2583 | 2221 | −518 | 1261 | −321 | O |
| ATOM | 667 | OD2 | ASP | A | 90 | −5.640 | 27.086 | 11.811 | 1.00 | 29.66 | | O |
| ANISOU | 667 | OD2 | ASP | A | 90 | 3481 | 4713 | 3076 | −50 | −1385 | −757 | O |
| ATOM | 668 | N | ASP | A | 91 | −7.148 | 26.017 | 16.047 | 1.00 | 13.48 | | N |
| ANISOU | 668 | N | ASP | A | 91 | 952 | 2085 | 2086 | 285 | −136 | 487 | N |
| ATOM | 669 | CA | ASP | A | 91 | −8.377 | 25.208 | 16.013 | 1.00 | 13.45 | | C |
| ANISOU | 669 | CA | ASP | A | 91 | 985 | 2199 | 1925 | 240 | 32 | 236 | C |
| ATOM | 670 | C | ASP | A | 91 | −8.687 | 24.648 | 17.373 | 1.00 | 12.52 | | C |
| ANISOU | 670 | C | ASP | A | 91 | 1039 | 1936 | 1781 | 183 | −1 | 82 | C |
| ATOM | 671 | O | ASP | A | 91 | −8.240 | 23.577 | 17.798 | 1.00 | 11.72 | | O |
| ANISOU | 671 | O | ASP | A | 91 | 895 | 1875 | 1683 | 180 | −68 | −74 | O |
| ATOM | 672 | CB | ASP | A | 91 | −8.213 | 24.082 | 15.003 | 1.00 | 15.41 | | C |
| ANISOU | 672 | CB | ASP | A | 91 | 1599 | 2557 | 1698 | 145 | −114 | 89 | C |
| ATOM | 673 | CG | ASP | A | 91 | −9.326 | 23.075 | 14.848 | 1.00 | 17.12 | | C |
| ANISOU | 673 | CG | ASP | A | 91 | 1326 | 2903 | 2277 | 225 | −848 | −139 | C |
| ATOM | 674 | OD1 | ASP | A | 91 | −10.295 | 23.176 | 15.592 | 1.00 | 24.40 | | O |
| ANISOU | 674 | OD1 | ASP | A | 91 | 1444 | 5018 | 2809 | −378 | −369 | 206 | O |
| ATOM | 675 | OD2 | ASP | A | 91 | −9.237 | 22.138 | 14.022 | 1.00 | 26.88 | | O |
| ANISOU | 675 | OD2 | ASP | A | 91 | 3094 | 3396 | 3724 | 207 | −1281 | −1071 | O |
| ATOM | 676 | N | SER | A | 92 | −9.532 | 25.377 | 18.107 | 1.00 | 12.70 | | N |
| ANISOU | 676 | N | SER | A | 92 | 1180 | 1794 | 1851 | 315 | −40 | 243 | N |
| ATOM | 677 | CA | SER | A | 92 | −9.862 | 24.966 | 19.473 | 1.00 | 12.09 | | C |
| ANISOU | 677 | CA | SER | A | 92 | 1544 | 1263 | 1786 | 97 | 189 | −95 | C |
| ATOM | 678 | C | SER | A | 92 | −10.582 | 23.629 | 19.422 | 1.00 | 12.24 | | C |
| ANISOU | 678 | C | SER | A | 92 | 1054 | 1561 | 2036 | 24 | −291 | 122 | C |
| ATOM | 679 | O | SER | A | 92 | −10.416 | 22.845 | 20.372 | 1.00 | 12.57 | | O |
| ANISOU | 679 | O | SER | A | 92 | 1229 | 1597 | 1948 | −161 | −338 | 160 | O |
| ATOM | 680 | CB | SER | A | 92 | −10.646 | 26.034 | 20.226 | 1.00 | 16.43 | | C |
| ANISOU | 680 | CB | SER | A | 92 | 1848 | 1853 | 2540 | 643 | 283 | −293 | C |
| ATOM | 681 | OG | SER | A | 92 | −11.881 | 26.217 | 19.611 | 1.00 | 17.10 | | O |
| ANISOU | 681 | OG | SER | A | 92 | 1246 | 2071 | 3180 | −36 | 491 | 128 | O |
| ATOM | 682 | N | GLN | A | 93 | −11.354 | 23.300 | 18.400 | 1.00 | 10.24 | | N |
| ANISOU | 682 | N | GLN | A | 93 | 931 | 1224 | 1737 | 410 | −56 | 102 | N |
| ATOM | 683 | CA | GLN | A | 93 | −12.035 | 22.029 | 18.377 | 1.00 | 11.19 | | C |
| ANISOU | 683 | CA | GLN | A | 93 | 1208 | 1349 | 1694 | 314 | −247 | −276 | C |
| ATOM | 684 | C | GLN | A | 93 | −11.017 | 20.893 | 18.398 | 1.00 | 10.10 | | C |
| ANISOU | 684 | C | GLN | A | 93 | 1276 | 1129 | 1432 | 192 | 113 | −112 | C |
| ATOM | 685 | O | GLN | A | 93 | −11.187 | 19.855 | 19.096 | 1.00 | 10.75 | | O |
| ANISOU | 685 | O | GLN | A | 93 | 658 | 1435 | 1991 | −181 | −442 | 317 | O |
| ATOM | 686 | CB | GLN | A | 93 | −12.989 | 21.896 | 17.184 | 1.00 | 15.89 | | C |
| ANISOU | 686 | CB | GLN | A | 93 | 1389 | 2711 | 1937 | 278 | −500 | −506 | C |
| ATOM | 687 | CG | GLN | A | 93 | −13.655 | 20.520 | 17.217 | 1.00 | 19.50 | | C |
| ANISOU | 687 | CG | GLN | A | 93 | 1162 | 3681 | 2566 | −636 | −736 | −222 | C |
| ATOM | 688 | CD | GLN | A | 93 | −14.608 | 20.414 | 18.406 | 1.00 | 21.01 | | C |
| ANISOU | 688 | CD | GLN | A | 93 | 1135 | 3934 | 2914 | −122 | −458 | −154 | C |
| ATOM | 689 | OE1 | GLN | A | 93 | −15.740 | 20.917 | 18.359 | 1.00 | 25.75 | | O |
| ANISOU | 689 | OE1 | GLN | A | 93 | 1438 | 3664 | 4683 | 153 | −78 | 49 | O |
| ATOM | 690 | NE2 | GLN | A | 93 | −14.173 | 19.779 | 19.498 | 1.00 | 19.87 | | N |
| ANISOU | 690 | NE2 | GLN | A | 93 | 1955 | 2370 | 3223 | −659 | −146 | 173 | N |
| ATOM | 691 | N | SER | A | 94 | −9.918 | 21.038 | 17.656 | 1.00 | 9.65 | | N |
| ANISOU | 691 | N | SER | A | 94 | 809 | 1547 | 1312 | 37 | −293 | 28 | N |
| ATOM | 692 | CA | SER | A | 94 | −8.920 | 19.961 | 17.636 | 1.00 | 9.78 | | C |
| ANISOU | 692 | CA | SER | A | 94 | 978 | 1634 | 1105 | 170 | −236 | −133 | C |
| ATOM | 693 | C | SER | A | 94 | −8.275 | 19.780 | 18.999 | 1.00 | 8.16 | | C |
| ANISOU | 693 | C | SER | A | 94 | 695 | 1277 | 1131 | 84 | −119 | −119 | C |
| ATOM | 694 | O | SER | A | 94 | −7.926 | 18.671 | 19.392 | 1.00 | 9.07 | | O |
| ANISOU | 694 | O | SER | A | 94 | 757 | 1291 | 1400 | −65 | −292 | −36 | O |
| ATOM | 695 | CB | SER | A | 94 | −7.845 | 20.236 | 16.582 | 1.00 | 10.88 | | C |
| ANISOU | 695 | CB | SER | A | 94 | 1152 | 1977 | 1002 | −79 | −136 | −489 | C |
| ATOM | 696 | OG | SER | A | 94 | −6.959 | 21.291 | 16.909 | 1.00 | 10.53 | | O |
| ANISOU | 696 | OG | SER | A | 94 | 929 | 1801 | 1269 | 117 | −294 | −162 | O |
| ATOM | 697 | N | PHE | A | 95 | −8.095 | 20.861 | 19.745 | 1.00 | 8.95 | | N |
| ANISOU | 697 | N | PHE | A | 95 | 718 | 1397 | 1284 | 62 | −128 | −263 | N |
| ATOM | 698 | CA | PHE | A | 95 | −7.594 | 20.815 | 21.118 | 1.00 | 8.61 | | C |
| ANISOU | 698 | CA | PHE | A | 95 | 695 | 1247 | 1329 | −152 | −255 | −93 | C |
| ATOM | 699 | C | PHE | A | 95 | −8.573 | 20.151 | 22.064 | 1.00 | 8.46 | | C |
| ANISOU | 699 | C | PHE | A | 95 | 799 | 1165 | 1251 | 101 | 45 | −381 | C |
| ATOM | 700 | O | PHE | A | 95 | −8.237 | 19.274 | 22.867 | 1.00 | 8.80 | | O |
| ANISOU | 700 | O | PHE | A | 95 | 841 | 1510 | 992 | −114 | −149 | −232 | O |
| ATOM | 701 | CB | PHE | A | 95 | −7.264 | 22.225 | 21.577 | 1.00 | 9.23 | | C |
| ANISOU | 701 | CB | PHE | A | 95 | 998 | 1264 | 1244 | −16 | −170 | −134 | C |
| ATOM | 702 | CG | PHE | A | 95 | −6.836 | 22.348 | 23.001 | 1.00 | 8.71 | | C |
| ANISOU | 702 | CG | PHE | A | 95 | 902 | 1186 | 1221 | −61 | −87 | −124 | C |
| ATOM | 703 | CD1 | PHE | A | 95 | −5.611 | 21.812 | 23.378 | 1.00 | 9.14 | | C |
| ANISOU | 703 | CD1 | PHE | A | 95 | 817 | 1399 | 1257 | −43 | −148 | −333 | C |
| ATOM | 704 | CD2 | PHE | A | 95 | −7.639 | 22.999 | 23.925 | 1.00 | 9.91 | | C |
| ANISOU | 704 | CD2 | PHE | A | 95 | 1189 | 1266 | 1310 | 218 | −29 | −43 | C |
| ATOM | 705 | CE1 | PHE | A | 95 | −5.184 | 21.924 | 24.671 | 1.00 | 10.44 | | C |
| ANISOU | 705 | CE1 | PHE | A | 95 | 1157 | 1476 | 1333 | −381 | −298 | −101 | C |
| ATOM | 706 | CE2 | PHE | A | 95 | −7.235 | 23.087 | 25.239 | 1.00 | 11.28 | | C |
| ANISOU | 706 | CE2 | PHE | A | 95 | 1281 | 1836 | 1170 | −384 | 201 | −117 | C |
| ATOM | 707 | CZ | PHE | A | 95 | −6.016 | 22.550 | 25.557 | 1.00 | 11.45 | | C |
| ANISOU | 707 | CZ | PHE | A | 95 | 1248 | 2088 | 1016 | −575 | −138 | −127 | C |
| ATOM | 708 | N | GLN | A | 96 | −9.847 | 20.497 | 21.954 | 1.00 | 9.89 | | N |
| ANISOU | 708 | N | GLN | A | 96 | 794 | 1478 | 1485 | 138 | 97 | −391 | N |
| ATOM | 709 | CA | GLN | A | 96 | −10.888 | 19.906 | 22.798 | 1.00 | 11.12 | | C |
| ANISOU | 709 | CA | GLN | A | 96 | 865 | 1743 | 1615 | 28 | 202 | −516 | C |
| ATOM | 710 | C | GLN | A | 96 | −11.001 | 18.389 | 22.601 | 1.00 | 10.39 | | C |
| ANISOU | 710 | C | GLN | A | 96 | 905 | 1670 | 1373 | −105 | −41 | −317 | C |
| ATOM | 711 | O | GLN | A | 96 | −11.353 | 17.675 | 23.531 | 1.00 | 13.40 | | O |
| ANISOU | 711 | O | GLN | A | 96 | 1497 | 2139 | 1454 | −637 | 385 | −388 | O |
| ATOM | 712 | CB | GLN | A | 96 | −12.271 | 20.504 | 22.554 | 1.00 | 12.09 | | C |
| ANISOU | 712 | CB | GLN | A | 96 | 804 | 1814 | 1975 | −9 | 87 | 50 | C |
| ATOM | 713 | CG | GLN | A | 96 | −12.349 | 21.963 | 22.856 | 1.00 | 15.43 | | C |
| ANISOU | 713 | CG | GLN | A | 96 | 1601 | 1944 | 2319 | 370 | 173 | −168 | C |
| ATOM | 714 | CD | GLN | A | 96 | −12.214 | 22.192 | 24.363 | 1.00 | 17.26 | | C |
| ANISOU | 714 | CD | GLN | A | 96 | 1895 | 2292 | 2371 | 39 | 257 | −364 | C |
| ATOM | 715 | OE1 | GLN | A | 96 | −13.068 | 21.716 | 25.143 | 1.00 | 21.13 | | O |
| ANISOU | 715 | OE1 | GLN | A | 96 | 2507 | 3028 | 2493 | −154 | 575 | −271 | O |
| ATOM | 716 | NE2 | GLN | A | 96 | −11.153 | 22.909 | 24.657 | 1.00 | 26.74 | | N |
| ANISOU | 716 | NE2 | GLN | A | 96 | 2562 | 4793 | 2805 | −1124 | −254 | −281 | N |
| ATOM | 717 | N | ASN | A | 97 | −10.627 | 17.934 | 21.406 | 1.00 | 8.29 | | N |
| ANISOU | 717 | N | ASN | A | 97 | 805 | 1136 | 1208 | 39 | −146 | −18 | N |
| ATOM | 718 | CA | ASN | A | 97 | −10.662 | 16.500 | 21.159 | 1.00 | 8.10 | | C |
| ANISOU | 718 | CA | ASN | A | 97 | 703 | 1105 | 1270 | −155 | −149 | 4 | C |
| ATOM | 719 | C | ASN | A | 97 | −9.510 | 15.663 | 21.626 | 1.00 | 8.18 | | C |
| ANISOU | 719 | C | ASN | A | 97 | 862 | 1161 | 1086 | 84 | −187 | −256 | C |
| ATOM | 720 | O | ASN | A | 97 | −9.532 | 14.455 | 21.494 | 1.00 | 9.74 | | O |
| ANISOU | 720 | O | ASN | A | 97 | 895 | 1230 | 1576 | 121 | −133 | −348 | O |
| ATOM | 721 | CB | ASN | A | 97 | −10.877 | 16.336 | 19.635 | 1.00 | 10.41 | | C |
| ANISOU | 721 | CB | ASN | A | 97 | 813 | 1718 | 1424 | 50 | −418 | −307 | C |
| ATOM | 722 | CG | ASN | A | 97 | −12.288 | 16.625 | 19.126 | 1.00 | 10.41 | | C |
| ANISOU | 722 | CG | ASN | A | 97 | 736 | 1677 | 1541 | −89 | −405 | −86 | C |
| ATOM | 723 | OD1 | ASN | A | 97 | −12.461 | 16.990 | 17.951 | 1.00 | 14.58 | | O |
| ANISOU | 723 | OD1 | ASN | A | 97 | 1382 | 2514 | 1644 | 31 | −574 | 155 | O |
| ATOM | 724 | ND2 | ASN | A | 97 | −13.320 | 16.461 | 19.903 | 1.00 | 15.08 | | N |
| ANISOU | 724 | ND2 | ASN | A | 97 | 782 | 3403 | 1546 | −256 | −229 | −696 | N |
| ATOM | 725 | N | LEU | A | 98 | −8.512 | 16.287 | 22.279 | 1.00 | 9.27 | | N |
| ANISOU | 725 | N | LEU | A | 98 | 892 | 1220 | 1408 | 89 | −357 | −243 | N |
| ATOM | 726 | CA | LEU | A | 98 | −7.373 | 15.473 | 22.733 | 1.00 | 8.48 | | C |
| ANISOU | 726 | CA | LEU | A | 98 | 761 | 1258 | 1204 | 83 | −162 | −240 | C |
| ATOM | 727 | C | LEU | A | 98 | −7.773 | 14.344 | 23.672 | 1.00 | 10.20 | | C |
| ANISOU | 727 | C | LEU | A | 98 | 730 | 1491 | 1656 | −59 | −313 | 87 | C |
| ATOM | 728 | O | LEU | A | 98 | −7.258 | 13.244 | 23.611 | 1.00 | 10.07 | | O |
| ANISOU | 728 | O | LEU | A | 98 | 891 | 1461 | 1474 | −163 | −249 | 122 | O |
| ATOM | 729 | CB | LEU | A | 98 | −6.354 | 16.355 | 23.467 | 1.00 | 10.35 | | C |
| ANISOU | 729 | CB | LEU | A | 98 | 743 | 1716 | 1475 | −222 | −152 | −279 | C |
| ATOM | 730 | CG | LEU | A | 98 | −5.568 | 17.319 | 22.580 | 1.00 | 9.08 | | C |
| ANISOU | 730 | CG | LEU | A | 98 | 649 | 1394 | 1406 | 87 | −228 | −151 | C |
| ATOM | 731 | CD1 | LEU | A | 98 | −4.647 | 18.158 | 23.444 | 1.00 | 12.16 | | C |
| ANISOU | 731 | CD1 | LEU | A | 98 | 1050 | 1700 | 1869 | −398 | −299 | −127 | C |
| ATOM | 732 | CD2 | LEU | A | 98 | −4.774 | 16.591 | 21.524 | 1.00 | 12.37 | | C |
| ANISOU | 732 | CD2 | LEU | A | 98 | 1324 | 1208 | 2168 | 107 | 480 | −214 | C |
| ATOM | 733 | N | SER | A | 99 | −8.658 | 14.644 | 24.624 | 1.00 | 10.22 | | N |
| ANISOU | 733 | N | SER | A | 99 | 843 | 1440 | 1599 | −217 | −233 | 128 | N |
| ATOM | 734 | CA | SER | A | 99 | −9.054 | 13.607 | 25.573 | 1.00 | 10.28 | | C |
| ANISOU | 734 | CA | SER | A | 99 | 1265 | 1282 | 1359 | −65 | −397 | 44 | C |
| ATOM | 735 | C | SER | A | 99 | −9.661 | 12.398 | 24.878 | 1.00 | 10.21 | | C |
| ANISOU | 735 | C | SER | A | 99 | 1056 | 1272 | 1553 | −157 | −231 | −52 | C |
| ATOM | 736 | O | SER | A | 99 | −9.373 | 11.266 | 25.249 | 1.00 | 11.08 | | O |
| ANISOU | 736 | O | SER | A | 99 | 935 | 1346 | 1928 | −215 | 56 | 222 | O |
| ATOM | 737 | CB | SER | A | 99 | −10.016 | 14.158 | 26.623 | 1.00 | 11.90 | | C |
| ANISOU | 737 | CB | SER | A | 99 | 1515 | 1806 | 1201 | −427 | −197 | −216 | C |
| ATOM | 738 | OG | SER | A | 99 | −10.471 | 13.138 | 27.480 | 1.00 | 20.72 | | O |
| ANISOU | 738 | OG | SER | A | 99 | 2697 | 2884 | 2292 | −649 | 385 | 583 | O |
| ATOM | 739 | N | ASN | A | 100 | −10.515 | 12.679 | 23.920 | 1.00 | 9.64 | | N |
| ANISOU | 739 | N | ASN | A | 100 | 659 | 1358 | 1647 | −205 | −99 | −88 | N |
| ATOM | 740 | CA | ASN | A | 100 | −11.124 | 11.579 | 23.174 | 1.00 | 10.83 | | C |
| ANISOU | 740 | CA | ASN | A | 100 | 763 | 1576 | 1776 | −329 | −111 | −206 | C |
| ATOM | 741 | C | ASN | A | 100 | −10.136 | 10.796 | 22.341 | 1.00 | 9.64 | | C |
| ANISOU | 741 | C | ASN | A | 100 | 924 | 1482 | 1257 | −257 | −204 | −11 | C |
| ATOM | 742 | O | ASN | A | 100 | −10.257 | 9.577 | 22.235 | 1.00 | 9.85 | | O |
| ANISOU | 742 | O | ASN | A | 100 | 926 | 1474 | 1345 | −210 | −94 | 86 | O |
| ATOM | 743 | CB | ASN | A | 100 | −12.269 | 12.107 | 22.319 | 1.00 | 14.62 | | C |
| ANISOU | 743 | CB | ASN | A | 100 | 1740 | 1242 | 2573 | 331 | −930 | −924 | C |
| ATOM | 744 | CG | ASN | A | 100 | −13.509 | 12.486 | 23.088 | 1.00 | 24.42 | | C |
| ANISOU | 744 | CG | ASN | A | 100 | 1481 | 2487 | 5310 | 784 | −945 | −2596 | C |
| ATOM | 745 | OD1 | ASN | A | 100 | −13.680 | 12.027 | 24.209 | 1.00 | 29.04 | | O |
| ANISOU | 745 | OD1 | ASN | A | 100 | 1164 | 4071 | 5797 | −750 | 1135 | −2719 | O |
| ATOM | 746 | ND2 | ASN | A | 100 | −14.337 | 13.322 | 22.479 | 1.00 | 40.70 | | N |
| ANISOU | 746 | ND2 | ASN | A | 100 | 2268 | 2867 | 10331 | 1468 | −3208 | −3127 | N |
| ATOM | 747 | N | TRP | A | 101 | −9.144 | 11.445 | 21.796 | 1.00 | 10.21 | | N |
| ANISOU | 747 | N | TRP | A | 101 | 1019 | 1205 | 1657 | −47 | 56 | 84 | N |
| ATOM | 748 | CA | TRP | A | 101 | −8.071 | 10.726 | 21.107 | 1.00 | 9.41 | | C |
| ANISOU | 748 | CA | TRP | A | 101 | 1035 | 1184 | 1356 | −169 | −100 | −119 | C |
| ATOM | 749 | C | TRP | A | 101 | −7.280 | 9.832 | 22.068 | 1.00 | 9.54 | | C |
| ANISOU | 749 | C | TRP | A | 101 | 907 | 1428 | 1288 | 21 | 98 | −45 | C |
| ATOM | 750 | O | TRP | A | 101 | −6.952 | 8.700 | 21.725 | 1.00 | 9.45 | | O |
| ANISOU | 750 | O | TRP | A | 101 | 874 | 1362 | 1356 | −67 | −105 | −78 | O |
| ATOM | 751 | CB | TRP | A | 101 | −7.120 | 11.726 | 20.414 | 1.00 | 10.87 | | C |
| ANISOU | 751 | CB | TRP | A | 101 | 1131 | 1533 | 1466 | −159 | 87 | 119 | C |
| ATOM | 752 | CG | TRP | A | 101 | −7.695 | 12.189 | 19.106 | 1.00 | 10.44 | | C |
| ANISOU | 752 | CG | TRP | A | 101 | 924 | 1526 | 1517 | −31 | 149 | 66 | C |
| ATOM | 753 | CD1 | TRP | A | 101 | −8.092 | 13.447 | 18.802 | 1.00 | 11.71 | | C |
| ANISOU | 753 | CD1 | TRP | A | 101 | 1384 | 1564 | 1502 | −52 | 206 | 284 | C |
| ATOM | 754 | CD2 | TRP | A | 101 | −7.904 | 11.382 | 17.937 | 1.00 | 11.15 | | C |
| ANISOU | 754 | CD2 | TRP | A | 101 | 939 | 1812 | 1484 | −203 | 208 | −50 | C |
| ATOM | 755 | NE1 | TRP | A | 101 | −8.569 | 13.483 | 17.491 | 1.00 | 12.76 | | N |
| ANISOU | 755 | NE1 | TRP | A | 101 | 1229 | 2075 | 1543 | 12 | 127 | 310 | N |
| ATOM | 756 | CE2 | TRP | A | 101 | −8.457 | 12.225 | 16.950 | 1.00 | 12.88 | | C |
| ANISOU | 756 | CE2 | TRP | A | 101 | 1210 | 2193 | 1489 | −265 | 181 | 168 | C |
| ATOM | 757 | CE3 | TRP | A | 101 | −7.696 | 10.047 | 17.616 | 1.00 | 12.19 | | C |
| ANISOU | 757 | CE3 | TRP | A | 101 | 918 | 1882 | 1832 | −352 | 161 | −289 | C |
| ATOM | 758 | CZ2 | TRP | A | 101 | −8.790 | 11.749 | 15.692 | 1.00 | 13.84 | | C |
| ANISOU | 758 | CZ2 | TRP | A | 101 | 1566 | 2043 | 1648 | −508 | −77 | 313 | C |
| ATOM | 759 | CZ3 | TRP | A | 101 | −8.024 | 9.557 | 16.371 | 1.00 | 13.62 | | C |
| ANISOU | 759 | CZ3 | TRP | A | 101 | 1665 | 1864 | 1646 | −288 | 147 | −182 | C |
| ATOM | 760 | CH2 | TRP | A | 101 | −8.569 | 10.444 | 15.433 | 1.00 | 14.97 | | C |
| ANISOU | 760 | CH2 | TRP | A | 101 | 1507 | 2198 | 1983 | −451 | −43 | −12 | C |
| ATOM | 761 | N | LYS | A | 102 | −7.021 | 10.344 | 23.273 | 1.00 | 10.20 | | N |
| ANISOU | 761 | N | LYS | A | 102 | 988 | 1466 | 1422 | −17 | −148 | −144 | N |
| ATOM | 762 | CA | LYS | A | 102 | −6.318 | 9.551 | 24.266 | 1.00 | 10.39 | | C |
| ANISOU | 762 | CA | LYS | A | 102 | 828 | 1519 | 1601 | −16 | −143 | −11 | C |
| ATOM | 763 | C | LYS | A | 102 | −7.147 | 8.316 | 24.594 | 1.00 | 10.03 | | C |
| ANISOU | 763 | C | LYS | A | 102 | 869 | 1315 | 1626 | 23 | −58 | −265 | C |
| ATOM | 764 | O | LYS | A | 102 | −6.650 | 7.189 | 24.687 | 1.00 | 9.34 | | O |
| ANISOU | 764 | O | LYS | A | 102 | 975 | 1426 | 1148 | 50 | −190 | −368 | O |
| ATOM | 765 | CB | LYS | A | 102 | −6.049 | 10.401 | 25.512 | 1.00 | 10.21 | | C |
| ANISOU | 765 | CB | LYS | A | 102 | 746 | 1576 | 1555 | −430 | −195 | 72 | C |
| ATOM | 766 | CG | LYS | A | 102 | −5.418 | 9.669 | 26.671 | 1.00 | 11.55 | | C |
| ANISOU | 766 | CG | LYS | A | 102 | 1245 | 1439 | 1705 | −147 | −282 | 89 | C |
| ATOM | 767 | CD | LYS | A | 102 | −5.103 | 10.549 | 27.883 | 1.00 | 14.07 | | C |
| ANISOU | 767 | CD | LYS | A | 102 | 1313 | 2375 | 1656 | −713 | −417 | 10 | C |
| ATOM | 768 | CE | LYS | A | 102 | −4.650 | 9.747 | 29.077 | 1.00 | 19.49 | | C |
| ANISOU | 768 | CE | LYS | A | 102 | 3725 | 1688 | 1992 | −456 | −994 | −87 | C |
| ATOM | 769 | NZ | LYS | A | 102 | −4.448 | 10.591 | 30.308 | 1.00 | 23.39 | | N |
| ANISOU | 769 | NZ | LYS | A | 102 | 3246 | 4013 | 1626 | −2105 | −534 | −420 | N |
| ATOM | 770 | N | LYS | A | 103 | −8.457 | 8.524 | 24.812 | 1.00 | 9.95 | | N |
| ANISOU | 770 | N | LYS | A | 103 | 750 | 1524 | 1505 | −24 | −156 | −104 | N |
| ATOM | 771 | CA | LYS | A | 103 | −9.334 | 7.400 | 25.089 | 1.00 | 9.76 | | C |
| ANISOU | 771 | CA | LYS | A | 103 | 945 | 1460 | 1305 | −50 | −160 | 35 | C |
| ATOM | 772 | C | LYS | A | 103 | −9.399 | 6.365 | 23.966 | 1.00 | 10.35 | | C |
| ANISOU | 772 | C | LYS | A | 103 | 921 | 1568 | 1442 | −100 | −97 | −78 | C |
| ATOM | 773 | O | LYS | A | 103 | −9.382 | 5.161 | 24.203 | 1.00 | 10.08 | | O |
| ANISOU | 773 | O | LYS | A | 103 | 719 | 1491 | 1621 | −6 | −127 | −148 | O |
| ATOM | 774 | CB | LYS | A | 103 | −10.771 | 7.901 | 25.385 | 1.00 | 9.84 | | C |
| ANISOU | 774 | CB | LYS | A | 103 | 917 | 1635 | 1187 | −202 | 22 | 78 | C |
| ATOM | 775 | CG | LYS | A | 103 | −10.898 | 8.625 | 26.715 | 1.00 | 11.42 | | C |
| ANISOU | 775 | CG | LYS | A | 103 | 1123 | 1774 | 1442 | −36 | −156 | −183 | C |
| ATOM | 776 | CD | LYS | A | 103 | −12.313 | 9.070 | 27.011 | 1.00 | 12.13 | | C |
| ANISOU | 776 | CD | LYS | A | 103 | 1223 | 1829 | 1556 | −40 | 84 | −103 | C |
| ATOM | 777 | CE | LYS | A | 103 | −12.421 | 9.852 | 28.293 | 1.00 | 16.17 | | C |
| ANISOU | 777 | CE | LYS | A | 103 | 1951 | 2478 | 1714 | −227 | 613 | −356 | C |
| ATOM | 778 | NZ | LYS | A | 103 | −13.796 | 9.991 | 28.788 | 1.00 | 20.95 | | N |
| ANISOU | 778 | NZ | LYS | A | 103 | 1988 | 3592 | 2378 | 604 | 580 | −544 | N |
| ATOM | 779 | N | GLU | A | 104 | −9.479 | 6.824 | 22.710 | 1.00 | 9.23 | | N |
| ANISOU | 779 | N | GLU | A | 104 | 655 | 1464 | 1389 | −127 | 39 | −251 | N |
| ATOM | 780 | CA | GLU | A | 104 | −9.505 | 5.894 | 21.569 | 1.00 | 9.89 | | C |
| ANISOU | 780 | CA | GLU | A | 104 | 1006 | 1283 | 1468 | 172 | −320 | −213 | C |
| ATOM | 781 | C | GLU | A | 104 | −8.247 | 5.041 | 21.512 | 1.00 | 9.54 | | C |
| ANISOU | 781 | C | GLU | A | 104 | 923 | 1392 | 1310 | 99 | −160 | −182 | C |
| ATOM | 782 | O | GLU | A | 104 | −8.293 | 3.842 | 21.304 | 1.00 | 9.87 | | O |
| ANISOU | 782 | O | GLU | A | 104 | 1014 | 1286 | 1450 | 148 | −232 | −204 | O |
| ATOM | 783 | CB | GLU | A | 104 | −9.666 | 6.727 | 20.282 | 1.00 | 11.17 | | C |
| ANISOU | 783 | CB | GLU | A | 104 | 1270 | 1532 | 1441 | 116 | −435 | −123 | C |
| ATOM | 784 | CG | GLU | A | 104 | −9.781 | 5.888 | 19.042 | 1.00 | 10.88 | | C |
| ANISOU | 784 | CG | GLU | A | 104 | 1296 | 1349 | 1488 | −165 | −372 | −21 | C |
| ATOM | 785 | CD | GLU | A | 104 | −9.894 | 6.643 | 17.733 | 1.00 | 10.69 | | C |
| ANISOU | 785 | CD | GLU | A | 104 | 1185 | 1533 | 1345 | 100 | −4 | −82 | C |
| ATOM | 786 | OE1 | GLU | A | 104 | −10.725 | 7.550 | 17.672 | 1.00 | 12.81 | | O |
| ANISOU | 786 | OE1 | GLU | A | 104 | 1437 | 1892 | 1539 | 449 | −437 | −75 | O |
| ATOM | 787 | OE2 | GLU | A | 104 | −9.123 | 6.276 | 16.828 | 1.00 | 15.70 | | O |
| ANISOU | 787 | OE2 | GLU | A | 104 | 1487 | 3093 | 1384 | 559 | −35 | −380 | O |
| ATOM | 788 | N | PHE | A | 105 | −7.101 | 5.695 | 21.693 | 1.00 | 8.71 | | N |
| ANISOU | 788 | N | PHE | A | 105 | 903 | 1319 | 1087 | 42 | 100 | 18 | N |
| ATOM | 789 | CA | PHE | A | 105 | −5.829 | 5.012 | 21.662 | 1.00 | 10.40 | | C |
| ANISOU | 789 | CA | PHE | A | 105 | 840 | 1750 | 1361 | 132 | 31 | 0 | C |
| ATOM | 790 | C | PHE | A | 105 | −5.810 | 3.964 | 22.783 | 1.00 | 9.18 | | C |
| ANISOU | 790 | C | PHE | A | 105 | 730 | 1440 | 1317 | −52 | −361 | −158 | C |
| ATOM | 791 | O | PHE | A | 105 | −5.516 | 2.800 | 22.564 | 1.00 | 10.84 | | O |
| ANISOU | 791 | O | PHE | A | 105 | 1026 | 1598 | 1495 | 220 | −27 | −268 | O |
| ATOM | 792 | CB | PHE | A | 105 | −4.636 | 5.947 | 21.792 | 1.00 | 10.07 | | C |
| ANISOU | 792 | CB | PHE | A | 105 | 842 | 1905 | 1081 | 69 | 74 | 272 | C |
| ATOM | 793 | CG | PHE | A | 105 | −3.342 | 5.144 | 21.854 | 1.00 | 9.95 | | C |
| ANISOU | 793 | CG | PHE | A | 105 | 838 | 1448 | 1495 | −43 | −114 | −51 | C |
| ATOM | 794 | CD1 | PHE | A | 105 | −2.792 | 4.592 | 20.709 | 1.00 | 10.99 | | C |
| ANISOU | 794 | CD1 | PHE | A | 105 | 980 | 1656 | 1540 | 124 | 25 | 98 | C |
| ATOM | 795 | CD2 | PHE | A | 105 | −2.672 | 4.944 | 23.056 | 1.00 | 11.80 | | C |
| ANISOU | 795 | CD2 | PHE | A | 105 | 753 | 2111 | 1619 | −9 | −254 | −132 | C |
| ATOM | 796 | CE1 | PHE | A | 105 | −1.638 | 3.866 | 20.728 | 1.00 | 12.03 | | C |
| ANISOU | 796 | CE1 | PHE | A | 105 | 1085 | 1793 | 1695 | 275 | −43 | −4 | C |
| ATOM | 797 | CE2 | PHE | A | 105 | −1.509 | 4.217 | 23.091 | 1.00 | 11.29 | | C |
| ANISOU | 797 | CE2 | PHE | A | 105 | 801 | 1767 | 1724 | −114 | −260 | 9 | C |
| ATOM | 798 | CZ | PHE | A | 105 | −0.996 | 3.685 | 21.927 | 1.00 | 12.16 | | C |
| ANISOU | 798 | CZ | PHE | A | 105 | 807 | 1890 | 1922 | 49 | −223 | −114 | C |
| ATOM | 799 | N | ILE | A | 106 | −6.120 | 4.378 | 24.024 | 1.00 | 8.07 | | N |
| ANISOU | 799 | N | ILE | A | 106 | 649 | 1082 | 1334 | 66 | −145 | −9 | N |
| ATOM | 800 | CA | ILE | A | 106 | −6.046 | 3.428 | 25.121 | 1.00 | 10.56 | | C |
| ANISOU | 800 | CA | ILE | A | 106 | 1010 | 1436 | 1567 | −101 | −251 | 246 | C |
| ATOM | 801 | C | ILE | A | 106 | −6.946 | 2.228 | 24.922 | 1.00 | 10.44 | | C |
| ANISOU | 801 | C | ILE | A | 106 | 1112 | 1299 | 1554 | −73 | −383 | 407 | C |
| ATOM | 802 | O | ILE | A | 106 | −6.577 | 1.094 | 25.212 | 1.00 | 11.85 | | O |
| ANISOU | 802 | O | ILE | A | 106 | 1257 | 1329 | 1915 | 61 | −424 | 321 | O |
| ATOM | 803 | CB | ILE | A | 106 | −6.328 | 4.174 | 26.457 | 1.00 | 10.79 | | C |
| ANISOU | 803 | CB | ILE | A | 106 | 1174 | 1562 | 1363 | −251 | −335 | 334 | C |
| ATOM | 804 | CG1 | ILE | A | 106 | −5.186 | 5.139 | 26.818 | 1.00 | 12.53 | | C |
| ANISOU | 804 | CG1 | ILE | A | 106 | 1165 | 1891 | 1705 | −340 | −409 | 154 | C |
| ATOM | 805 | CG2 | ILE | A | 106 | −6.654 | 3.241 | 27.580 | 1.00 | 13.66 | | C |
| ANISOU | 805 | CG2 | ILE | A | 106 | 1442 | 2141 | 1607 | −291 | −245 | 665 | C |
| ATOM | 806 | CD1 | ILE | A | 106 | −5.449 | 6.020 | 27.978 | 1.00 | 16.29 | | C |
| ANISOU | 806 | CD1 | ILE | A | 106 | 2140 | 2105 | 1945 | −790 | −136 | −144 | C |
| ATOM | 807 | N | TYR | A | 107 | −8.161 | 2.488 | 24.439 | 1.00 | 10.64 | | N |
| ANISOU | 807 | N | TYR | A | 107 | 1101 | 1264 | 1677 | 126 | −431 | −88 | N |
| ATOM | 808 | CA | TYR | A | 107 | −9.105 | 1.411 | 24.199 | 1.00 | 12.88 | | C |
| ANISOU | 808 | CA | TYR | A | 107 | 1064 | 1666 | 2165 | −140 | −134 | −110 | C |
| ATOM | 809 | C | TYR | A | 107 | −8.663 | 0.466 | 23.080 | 1.00 | 13.00 | | C |
| ANISOU | 809 | C | TYR | A | 107 | 1635 | 1227 | 2077 | −79 | −512 | −128 | C |
| ATOM | 810 | O | TYR | A | 107 | −8.574 | −0.755 | 23.233 | 1.00 | 17.43 | | O |
| ANISOU | 810 | O | TYR | A | 107 | 2538 | 1253 | 2832 | 11 | −998 | 1 | O |
| ATOM | 811 | CB | TYR | A | 107 | −10.485 | 2.006 | 23.851 | 1.00 | 13.31 | | C |
| ANISOU | 811 | CB | TYR | A | 107 | 894 | 2295 | 1869 | −223 | −51 | −3 | C |
| ATOM | 812 | CG | TYR | A | 107 | −11.457 | 0.882 | 23.600 | 1.00 | 16.35 | | C |
| ANISOU | 812 | CG | TYR | A | 107 | 1307 | 2008 | 2897 | −226 | −567 | 275 | C |
| ATOM | 813 | CD1 | TYR | A | 107 | −11.791 | −0.022 | 24.579 | 1.00 | 19.54 | | C |
| ANISOU | 813 | CD1 | TYR | A | 107 | 1833 | 2142 | 3449 | −449 | −804 | 73 | C |
| ATOM | 814 | CD2 | TYR | A | 107 | −12.059 | 0.734 | 22.361 | 1.00 | 22.47 | | C |
| ANISOU | 814 | CD2 | TYR | A | 107 | 1866 | 3254 | 3417 | −1383 | −1262 | 826 | C |
| ATOM | 815 | CE1 | TYR | A | 107 | −12.704 | −1.042 | 24.286 | 1.00 | 29.69 | | C |
| ANISOU | 815 | CE1 | TYR | A | 107 | 3838 | 3185 | 4257 | −1886 | −1570 | 1214 | C |
| ATOM | 816 | CE2 | TYR | A | 107 | −12.936 | −0.255 | 22.066 | 1.00 | 26.78 | | C |
| ANISOU | 816 | CE2 | TYR | A | 107 | 2349 | 3692 | 4132 | −1919 | −1558 | 1146 | C |
| ATOM | 817 | CZ | TYR | A | 107 | −13.278 | −1.165 | 23.039 | 1.00 | 28.50 | | C |
| ANISOU | 817 | CZ | TYR | A | 107 | 2669 | 3306 | 4854 | −1665 | −2135 | 1699 | C |
| ATOM | 818 | OH | TYR | A | 107 | −14.180 | −2.165 | 22.705 | 1.00 | 35.12 | | O |
| ANISOU | 818 | OH | TYR | A | 107 | 3134 | 4522 | 5687 | −2723 | −2282 | 2060 | O |
| ATOM | 819 | N | TYR | A | 108 | −8.352 | 0.995 | 21.896 | 1.00 | 12.50 | | N |
| ANISOU | 819 | N | TYR | A | 108 | 1423 | 1451 | 1878 | 99 | −718 | −227 | N |
| ATOM | 820 | CA | TYR | A | 108 | −8.072 | 0.133 | 20.769 | 1.00 | 13.30 | | C |
| ANISOU | 820 | CA | TYR | A | 108 | 1576 | 1444 | 2033 | 286 | −630 | −273 | C |
| ATOM | 821 | C | TYR | A | 108 | −6.656 | −0.406 | 20.773 | 1.00 | 16.01 | | C |
| ANISOU | 821 | C | TYR | A | 108 | 1867 | 1583 | 2631 | 728 | −990 | −440 | C |
| ATOM | 822 | O | TYR | A | 108 | −6.425 | −1.464 | 20.190 | 1.00 | 20.12 | | O |
| ANISOU | 822 | O | TYR | A | 108 | 2089 | 2288 | 3267 | 788 | −964 | −1226 | O |
| ATOM | 823 | CB | TYR | A | 108 | −8.361 | 0.834 | 19.438 | 1.00 | 12.92 | | C |
| ANISOU | 823 | CB | TYR | A | 108 | 1432 | 1556 | 1922 | 437 | −529 | −314 | C |
| ATOM | 824 | CG | TYR | A | 108 | −9.861 | 0.977 | 19.230 | 1.00 | 13.96 | | C |
| ANISOU | 824 | CG | TYR | A | 108 | 1449 | 1515 | 2340 | 459 | −681 | −630 | C |
| ATOM | 825 | CD1 | TYR | A | 108 | −10.656 | −0.158 | 19.025 | 1.00 | 14.35 | | C |
| ANISOU | 825 | CD1 | TYR | A | 108 | 1682 | 1659 | 2114 | 305 | −951 | −614 | C |
| ATOM | 826 | CD2 | TYR | A | 108 | −10.476 | 2.216 | 19.274 | 1.00 | 13.59 | | C |
| ANISOU | 826 | CD2 | TYR | A | 108 | 1565 | 1647 | 1951 | 618 | −535 | −736 | C |
| ATOM | 827 | CE1 | TYR | A | 108 | −12.029 | −0.068 | 18.844 | 1.00 | 15.94 | | C |
| ANISOU | 827 | CE1 | TYR | A | 108 | 1649 | 1601 | 2808 | 351 | −869 | −956 | C |
| ATOM | 828 | CE2 | TYR | A | 108 | −11.850 | 2.298 | 19.086 | 1.00 | 13.78 | | C |
| ANISOU | 828 | CE2 | TYR | A | 108 | 1556 | 1428 | 2250 | 500 | −360 | −327 | C |
| ATOM | 829 | CZ | TYR | A | 108 | −12.608 | 1.176 | 18.858 | 1.00 | 15.20 | | C |
| ANISOU | 829 | CZ | TYR | A | 108 | 1922 | 1597 | 2257 | 448 | −1164 | −664 | C |
| ATOM | 830 | OH | TYR | A | 108 | −13.953 | 1.346 | 18.721 | 1.00 | 15.25 | | O |
| ANISOU | 830 | OH | TYR | A | 108 | 1848 | 1561 | 2384 | 425 | −749 | −418 | O |
| ATOM | 831 | N | ALA | A | 109 | −5.688 | 0.233 | 21.407 | 1.00 | 13.07 | | N |
| ANISOU | 831 | N | ALA | A | 109 | 1334 | 1754 | 1877 | 540 | −221 | −403 | N |
| ATOM | 832 | CA | ALA | A | 109 | −4.353 | −0.312 | 21.533 | 1.00 | 13.09 | | C |
| ANISOU | 832 | CA | ALA | A | 109 | 1447 | 1458 | 2071 | 616 | −293 | −285 | C |
| ATOM | 833 | C | ALA | A | 109 | −4.331 | −1.302 | 22.697 | 1.00 | 16.43 | | C |
| ANISOU | 833 | C | ALA | A | 109 | 1715 | 1930 | 2598 | 381 | −436 | 231 | C |
| ATOM | 834 | O | ALA | A | 109 | −3.371 | −2.042 | 22.863 | 1.00 | 19.31 | | O |
| ANISOU | 834 | O | ALA | A | 109 | 2347 | 1918 | 3073 | 723 | −720 | 286 | O |
| ATOM | 835 | CB | ALA | A | 109 | −3.308 | 0.771 | 21.815 | 1.00 | 15.36 | | C |
| ANISOU | 835 | CB | ALA | A | 109 | 1550 | 2094 | 2190 | 227 | −680 | 229 | C |
| ATOM | 836 | N | ASP | A | 110 | −5.345 | −1.263 | 23.500 | 1.00 | 18.05 | | N |
| ANISOU | 836 | N | ASP | A | 110 | 2011 | 2502 | 2345 | 5 | −361 | 68 | N |
| ATOM | 837 | CA | ASP | A | 110 | −5.527 | −2.012 | 24.734 | 1.00 | 18.40 | | C |
| ANISOU | 837 | CA | ASP | A | 110 | 1839 | 2431 | 2719 | −268 | −468 | 271 | C |
| ATOM | 838 | C | ASP | A | 110 | −4.348 | −1.801 | 25.683 | 1.00 | 20.09 | | C |
| ANISOU | 838 | C | ASP | A | 110 | 2442 | 2089 | 3104 | 50 | −1056 | 374 | C |
| ATOM | 839 | O | ASP | A | 110 | −3.641 | −2.710 | 26.089 | 1.00 | 21.44 | | O |
| ANISOU | 839 | O | ASP | A | 110 | 2525 | 2102 | 3519 | 94 | −974 | 539 | O |
| ATOM | 840 | CB | ASP | A | 110 | −5.750 | −3.495 | 24.436 | 1.00 | 24.63 | | C |
| ANISOU | 840 | CB | ASP | A | 110 | 3488 | 2598 | 3271 | −751 | −672 | 116 | C |
| ATOM | 841 | CG | ASP | A | 110 | −6.185 | −4.256 | 25.674 | 1.00 | 26.88 | | C |
| ANISOU | 841 | CG | ASP | A | 110 | 3032 | 2960 | 4219 | −985 | −655 | 891 | C |
| ATOM | 842 | OD1 | ASP | A | 110 | −5.754 | −5.414 | 25.812 | 1.00 | 33.54 | | O |
| ANISOU | 842 | OD1 | ASP | A | 110 | 5516 | 2302 | 4926 | −1276 | −663 | 455 | O |
| ATOM | 843 | OD2 | ASP | A | 110 | −6.914 | −3.698 | 26.508 | 1.00 | 28.45 | | O |
| ANISOU | 843 | OD2 | ASP | A | 110 | 3147 | 3628 | 4036 | −787 | −283 | 1422 | O |
| ATOM | 844 | N | VAL | A | 111 | −4.184 | −0.503 | 26.007 | 1.00 | 17.19 | | N |
| ANISOU | 844 | N | VAL | A | 111 | 1573 | 2154 | 2803 | 55 | −482 | 174 | N |
| ATOM | 845 | CA | VAL | A | 111 | −3.064 | −0.168 | 26.896 | 1.00 | 18.36 | | C |
| ANISOU | 845 | CA | VAL | A | 111 | 1549 | 2837 | 2592 | −549 | −287 | 483 | C |
| ATOM | 846 | C | VAL | A | 111 | −3.359 | −0.659 | 28.301 | 1.00 | 20.64 | | C |
| ANISOU | 846 | C | VAL | A | 111 | 2494 | 2697 | 2650 | −128 | −322 | 657 | C |
| ATOM | 847 | O | VAL | A | 111 | −4.333 | −0.253 | 28.960 | 1.00 | 25.03 | | O |
| ANISOU | 847 | O | VAL | A | 111 | 2559 | 4297 | 2656 | −424 | 87 | 628 | O |
| ATOM | 848 | CB | VAL | A | 111 | −2.859 | 1.355 | 26.869 | 1.00 | 18.98 | | C |
| ANISOU | 848 | CB | VAL | A | 111 | 2189 | 2862 | 2161 | −707 | −602 | 444 | C |
| ATOM | 849 | CG1 | VAL | A | 111 | −1.740 | 1.709 | 27.805 | 1.00 | 23.84 | | C |
| ANISOU | 849 | CG1 | VAL | A | 111 | 2764 | 3160 | 3132 | −551 | −1430 | 253 | C |
| ATOM | 850 | CG2 | VAL | A | 111 | −2.544 | 1.823 | 25.452 | 1.00 | 19.45 | | C |
| ANISOU | 850 | CG2 | VAL | A | 111 | 1866 | 3064 | 2461 | 32 | −239 | 810 | C |
| ATOM | 851 | N | LYS | A | 112 | −2.551 | −1.574 | 28.819 | 1.00 | 25.16 | | N |
| ANISOU | 851 | N | LYS | A | 112 | 2919 | 2676 | 3966 | −598 | −1390 | 1085 | N |
| ATOM | 852 | CA | LYS | A | 112 | −2.942 | −2.191 | 30.093 | 1.00 | 35.81 | | C |
| ANISOU | 852 | CA | LYS | A | 112 | 5889 | 3793 | 3924 | −1409 | −2041 | 1778 | C |
| ATOM | 853 | C | LYS | A | 112 | −2.598 | −1.307 | 31.280 | 1.00 | 37.58 | | C |
| ANISOU | 853 | C | LYS | A | 112 | 5637 | 4724 | 3917 | −1723 | −1429 | 1248 | C |
| ATOM | 854 | O | LYS | A | 112 | −3.291 | −1.436 | 32.303 | 1.00 | 47.23 | | O |
| ANISOU | 854 | O | LYS | A | 112 | 5065 | 8622 | 4259 | −2219 | −1288 | 730 | O |
| ATOM | 855 | CB | LYS | A | 112 | −2.327 | −3.593 | 30.179 | 1.00 | 43.75 | | C |
| ANISOU | 855 | CB | LYS | A | 112 | 8011 | 3482 | 5129 | −1376 | −3742 | 1992 | C |
| ATOM | 856 | CG | LYS | A | 112 | −3.045 | −4.585 | 29.238 | 1.00 | 50.58 | | C |
| ANISOU | 856 | CG | LYS | A | 112 | 9317 | 4244 | 5656 | −1610 | −3562 | 1029 | C |
| ATOM | 857 | CD | LYS | A | 112 | −2.410 | −5.967 | 29.320 | 1.00 | 49.22 | | C |
| ANISOU | 857 | CD | LYS | A | 112 | 6767 | 4706 | 7229 | −1574 | −1746 | −165 | C |
| ATOM | 858 | CE | LYS | A | 112 | −3.185 | −7.017 | 28.533 | 1.00 | 46.45 | | C |
| ANISOU | 858 | CE | LYS | A | 112 | 5047 | 4742 | 7860 | −2518 | −283 | −47 | C |
| ATOM | 859 | NZ | LYS | A | 112 | −4.399 | −7.379 | 29.317 | 1.00 | 58.31 | | N |
| ANTSOU | 859 | NZ | LYS | A | 112 | 4372 | 9506 | 8276 | −1688 | 654 | −1500 | N |
| ATOM | 860 | N | GLU | A | 113 | −1.602 | −0.444 | 31.127 | 1.00 | 36.31 | | N |
| ANISOU | 860 | N | GLU | A | 113 | 5226 | 4331 | 4238 | −1379 | −1881 | 1437 | N |
| ATOM | 861 | CA | GLU | A | 113 | −1.175 | 0.465 | 32.191 | 1.00 | 30.82 | | C |
| ANISOU | 861 | CA | GLU | A | 113 | 3602 | 4367 | 3741 | −1176 | −266 | 1043 | C |
| ATOM | 862 | C | GLU | A | 113 | −1.185 | 1.914 | 31.732 | 1.00 | 28.02 | | C |
| ANISOU | 862 | C | GLU | A | 113 | 3162 | 4530 | 2955 | −1344 | −792 | 1166 | C |
| ATOM | 863 | O | GLU | A | 113 | −0.129 | 2.529 | 31.569 | 1.00 | 26.04 | | O |
| ANISOU | 863 | O | GLU | A | 113 | 3197 | 4393 | 2306 | −1252 | −351 | 743 | O |
| ATOM | 864 | CB | GLU | A | 113 | 0.231 | 0.088 | 32.657 | 1.00 | 42.36 | | C |
| ANTSOU | 864 | CB | GLU | A | 113 | 5680 | 5890 | 4526 | 390 | −2605 | 346 | C |
| ATOM | 865 | CG | GLU | A | 113 | 0.184 | −0.969 | 33.766 | 1.00 | 46.36 | | C |
| ANISOU | 865 | CG | GLU | A | 113 | 6255 | 6230 | 5129 | 829 | −1527 | 765 | C |
| ATOM | 866 | CD | GLU | A | 113 | −0.743 | −0.505 | 34.890 | 1.00 | 58.60 | | C |
| ANISOU | 866 | CD | GLU | A | 113 | 7472 | 8315 | 6477 | −513 | 149 | −73 | C |
| ATOM | 867 | OE1 | GLU | A | 113 | −0.805 | 0.725 | 35.137 | 1.00 | 63.69 | | O |
| ANISOU | 867 | OE1 | GLU | A | 113 | 6047 | 8537 | 9614 | 1523 | 844 | −428 | O |
| ATOM | 868 | OE2 | GLU | A | 113 | −1.418 | −1.347 | 35.534 | 1.00 | 75.86 | | O |
| ANISOU | 868 | OE2 | GLU | A | 113 | 9106 | 11086 | 8630 | −2865 | 1228 | −95 | O |
| ATOM | 869 | N | PRO | A | 114 | −2.366 | 2.451 | 31.501 | 1.00 | 28.06 | | N |
| ANISOU | 869 | N | PRO | A | 114 | 3160 | 4981 | 2522 | −1044 | −294 | 1293 | N |
| ATOM | 870 | CA | PRO | A | 114 | −2.509 | 3.724 | 30.804 | 1.00 | 29.04 | | C |
| ANISOU | 870 | CA | PRO | A | 114 | 3414 | 4951 | 2669 | −1048 | −678 | 1251 | C |
| ATOM | 871 | C | PRO | A | 114 | −1.911 | 4.923 | 31.521 | 1.00 | 27.08 | | C |
| ANISOU | 871 | C | PRO | A | 114 | 2917 | 4751 | 2620 | −328 | −506 | 893 | C |
| ATOM | 872 | O | PRO | A | 114 | −1.499 | 5.907 | 30.910 | 1.00 | 33.17 | | O |
| ANISOU | 872 | O | PRO | A | 114 | 4530 | 4974 | 3098 | −1295 | −967 | 983 | O |
| ATOM | 873 | CB | PRO | A | 114 | −4.029 | 3.944 | 30.723 | 1.00 | 31.89 | | C |
| ANISOU | 873 | CB | PRO | A | 114 | 3516 | 5249 | 3351 | −791 | −1013 | 934 | C |
| ATOM | 874 | CG | PRO | A | 114 | −4.673 | 2.868 | 31.503 | 1.00 | 32.56 | | C |
| ANISOU | 874 | CG | PRO | A | 114 | 3237 | 5402 | 3734 | −819 | −20 | 445 | C |
| ATOM | 875 | CD | PRO | A | 114 | −3.657 | 1.835 | 31.868 | 1.00 | 30.69 | | C |
| ANISOU | 875 | CD | PRO | A | 114 | 3177 | 5101 | 3381 | −1471 | −737 | 1067 | C |
| ATOM | 876 | N | GLU | A | 115 | −1.890 | 4.788 | 32.846 | 1.00 | 28.59 | | N |
| ANISOU | 876 | N | GLU | A | 115 | 2461 | 5925 | 2478 | −95 | 40 | 536 | N |
| ATOM | 877 | CA | GLU | A | 115 | −1.349 | 5.820 | 33.713 | 1.00 | 32.24 | | C |
| ANISOU | 877 | CA | GLU | A | 115 | 3323 | 6484 | 2441 | −616 | 833 | −21 | C |
| ATOM | 878 | C | GLU | A | 115 | 0.157 | 6.003 | 33.511 | 1.00 | 27.66 | | C |
| ANISOU | 878 | C | GLU | A | 115 | 3221 | 5006 | 2281 | −566 | 467 | 229 | C |
| ATOM | 879 | O | GLU | A | 115 | 0.629 | 7.091 | 33.864 | 1.00 | 36.00 | | O |
| ANISOU | 879 | O | GLU | A | 115 | 3302 | 5956 | 4421 | −112 | −730 | −1674 | O |
| ATOM | 880 | CB | GLU | A | 115 | −1.604 | 5.478 | 35.182 | 1.00 | 40.18 | | C |
| ANISOU | 880 | CB | GLU | A | 115 | 5318 | 7375 | 2574 | −1817 | 1605 | −318 | C |
| ATOM | 881 | CG | GLU | A | 115 | −2.883 | 4.722 | 35.485 | 1.00 | 49.68 | | C |
| ANISOU | 881 | CG | GLU | A | 115 | 6800 | 8432 | 3645 | −3256 | 1986 | −242 | C |
| ATOM | 882 | CD | GLU | A | 115 | −2.877 | 3.237 | 35.190 | 1.00 | 61.31 | | C |
| ANISOU | 882 | CD | GLU | A | 115 | 9960 | 8118 | 5216 | −3786 | 178 | 321 | C |
| ATOM | 883 | OE1 | GLU | A | 115 | −1.838 | 2.729 | 34.719 | 1.00 | 57.89 | | O |
| ANISOU | 883 | OE1 | GLU | A | 115 | 12159 | 5931 | 3906 | −3311 | 1415 | 506 | O |
| ATOM | 884 | OE2 | GLU | A | 115 | −3.918 | 2.569 | 35.388 | 1.00 | 74.27 | | O |
| ANISOU | 884 | OE2 | GLU | A | 115 | 10112 | 10017 | 8089 | −5475 | −1514 | −1723 | O |
| ATOM | 885 | N | SER | A | 116 | 0.854 | 4.994 | 33.001 | 1.00 | 24.15 | | N |
| ANISOU | 885 | N | SER | A | 116 | 2971 | 4415 | 1791 | −521 | −149 | 679 | N |
| ATOM | 886 | CA | SER | A | 116 | 2.299 | 5.010 | 32.791 | 1.00 | 23.05 | | C |
| ANISOU | 886 | CA | SER | A | 116 | 2853 | 3982 | 1924 | −477 | −622 | 109 | C |
| ATOM | 887 | C | SER | A | 116 | 2.695 | 5.123 | 31.325 | 1.00 | 20.48 | | C |
| ANISOU | 887 | C | SER | A | 116 | 2151 | 3606 | 2026 | −445 | −405 | −159 | C |
| ATOM | 888 | O | SER | A | 116 | 3.837 | 5.320 | 30.940 | 1.00 | 26.28 | | O |
| ANISOU | 888 | O | SER | A | 116 | 2243 | 5058 | 2683 | −1256 | −667 | 563 | O |
| ATOM | 889 | CB | SER | A | 116 | 2.918 | 3.751 | 33.426 | 1.00 | 27.75 | | C |
| ANISOU | 889 | CB | SER | A | 116 | 3379 | 4434 | 2731 | −370 | −935 | 593 | C |
| ATOM | 890 | OG | SER | A | 116 | 2.769 | 2.654 | 32.536 | 1.00 | 35.99 | | O |
| ANISOU | 890 | OG | SER | A | 116 | 3682 | 4219 | 5772 | −76 | −396 | −594 | O |
| ATOM | 891 | N | PHE | A | 117 | 1.757 | 4.978 | 30.397 | 1.00 | 17.90 | | N |
| ANISOU | 891 | N | PHE | A | 117 | 1919 | 3112 | 1771 | −505 | −280 | 626 | N |
| ATOM | 892 | CA | PHE | A | 117 | 2.074 | 4.923 | 28.978 | 1.00 | 16.47 | | C |
| ANISOU | 892 | CA | PHE | A | 117 | 2154 | 2256 | 1845 | −736 | −185 | 326 | C |
| ATOM | 893 | C | PHE | A | 117 | 2.496 | 6.306 | 28.503 | 1.00 | 14.91 | | C |
| ANISOU | 893 | C | PHE | A | 117 | 1653 | 2209 | 1803 | −504 | −587 | 500 | C |
| ATOM | 894 | O | PHE | A | 117 | 1.859 | 7.316 | 28.802 | 1.00 | 15.92 | | O |
| ANISOU | 894 | O | PHE | A | 117 | 2047 | 2298 | 1703 | −480 | −474 | 309 | O |
| ATOM | 895 | CB | PHE | A | 117 | 0.867 | 4.391 | 28.203 | 1.00 | 16.30 | | C |
| ANISOU | 895 | CB | PHE | A | 117 | 1948 | 2545 | 1701 | −885 | 207 | 116 | C |
| ATOM | 896 | CG | PHE | A | 117 | 1.216 | 4.140 | 26.740 | 1.00 | 14.70 | | C |
| ANISOU | 896 | CG | PHE | A | 117 | 1364 | 2451 | 1769 | −679 | 309 | 48 | C |
| ATOM | 897 | CD1 | PHE | A | 117 | 1.109 | 5.140 | 25.795 | 1.00 | 13.43 | | C |
| ANISOU | 897 | CD1 | PHE | A | 117 | 1303 | 2320 | 1478 | −931 | −228 | −166 | C |
| ATOM | 898 | CD2 | PHE | A | 117 | 1.667 | 2.884 | 26.394 | 1.00 | 17.32 | | C |
| ANISOU | 898 | CD2 | PHE | A | 117 | 1757 | 2377 | 2448 | −629 | 207 | −95 | C |
| ATOM | 899 | CE1 | PHE | A | 117 | 1.514 | 4.887 | 24.509 | 1.00 | 14.13 | | C |
| ANISOU | 899 | CE1 | PHE | A | 117 | 1024 | 2905 | 1440 | −678 | −296 | −127 | C |
| ATOM | 900 | CE2 | PHE | A | 117 | 2.063 | 2.623 | 25.090 | 1.00 | 16.71 | | C |
| ANISOU | 900 | CE2 | PHE | A | 117 | 1131 | 2824 | 2394 | −287 | −224 | −494 | C |
| ATOM | 901 | CZ | PHE | A | 117 | 1.990 | 3.639 | 24.163 | 1.00 | 15.70 | | C |
| ANISOU | 901 | CZ | PHE | A | 117 | 754 | 3259 | 1953 | −431 | −265 | −480 | C |
| ATOM | 902 | N | PRO | A | 118 | 3.540 | 6.403 | 27.718 | 1.00 | 14.03 | | N |
| ANISOU | 902 | N | PRO | A | 118 | 1827 | 2019 | 1484 | −529 | −600 | 455 | N |
| ATOM | 903 | CA | PRO | A | 118 | 4.007 | 7.730 | 27.317 | 1.00 | 15.71 | | C |
| ANISOU | 903 | CA | PRO | A | 118 | 1601 | 2207 | 2161 | −451 | −690 | 870 | C |
| ATOM | 904 | C | PRO | A | 118 | 3.196 | 8.353 | 26.191 | 1.00 | 12.54 | | C |
| ANISOU | 904 | C | PRO | A | 118 | 1582 | 1477 | 1704 | −134 | −600 | 96 | C |
| ATOM | 905 | O | PRO | A | 118 | 2.998 | 7.785 | 25.147 | 1.00 | 12.97 | | O |
| ANISOU | 905 | O | PRO | A | 118 | 1670 | 1672 | 1584 | 294 | −171 | −88 | O |
| ATOM | 906 | CB | PRO | A | 118 | 5.425 | 7.484 | 26.827 | 1.00 | 18.66 | | C |
| ANISOU | 906 | CB | PRO | A | 118 | 1492 | 2457 | 3141 | −320 | −624 | 1068 | C |
| ATOM | 907 | CG | PRO | A | 118 | 5.605 | 6.033 | 26.682 | 1.00 | 18.46 | | C |
| ANISOU | 907 | CG | PRO | A | 118 | 2090 | 2626 | 2298 | −836 | −106 | −521 | C |
| ATOM | 908 | CD | PRO | A | 118 | 4.417 | 5.317 | 27.234 | 1.00 | 15.60 | | C |
| ANISOU | 908 | CD | PRO | A | 118 | 2152 | 2251 | 1526 | −403 | −377 | 448 | C |
| ATOM | 909 | N | PHE | A | 119 | 2.744 | 9.575 | 26.412 | 1.00 | 11.23 | | N |
| ANISOU | 909 | N | PHE | A | 119 | 1429 | 1403 | 1434 | −414 | −371 | −19 | N |
| ATOM | 910 | CA | PHE | A | 119 | 2.143 | 10.449 | 25.455 | 1.00 | 9.85 | | C |
| ANISOU | 910 | CA | PHE | A | 119 | 1099 | 1206 | 1437 | −161 | −75 | −106 | C |
| ATOM | 911 | C | PHE | A | 119 | 3.007 | 11.722 | 25.365 | 1.00 | 9.13 | | C |
| ANISOU | 911 | C | PHE | A | 119 | 1208 | 1315 | 945 | −254 | −55 | −219 | C |
| ATOM | 912 | O | PHE | A | 119 | 3.574 | 12.179 | 26.385 | 1.00 | 11.10 | | O |
| ANISOU | 912 | O | PHE | A | 119 | 1546 | 1636 | 1033 | −375 | −364 | −188 | O |
| ATOM | 913 | CB | PHE | A | 119 | 0.709 | 10.872 | 25.822 | 1.00 | 11.82 | | C |
| ANISOU | 913 | CB | PHE | A | 119 | 1089 | 1822 | 1579 | −188 | 89 | −521 | C |
| ATOM | 914 | CG | PHE | A | 119 | −0.289 | 9.739 | 25.840 | 1.00 | 13.17 | | C |
| ANISOU | 914 | CG | PHE | A | 119 | 1352 | 2286 | 1365 | −547 | 369 | −575 | C |
| ATOM | 915 | CD1 | PHE | A | 119 | −1.006 | 9.441 | 24.671 | 1.00 | 13.39 | | C |
| ANISOU | 915 | CD1 | PHE | A | 119 | 1104 | 2328 | 1654 | −425 | 47 | −375 | C |
| ATOM | 916 | CD2 | PHE | A | 119 | −0.505 | 8.980 | 26.951 | 1.00 | 14.14 | | C |
| ANISOU | 916 | CD2 | PHE | A | 119 | 1792 | 2189 | 1393 | −511 | 360 | −543 | C |
| ATOM | 917 | CE1 | PHE | A | 119 | −1.907 | 8.397 | 24.678 | 1.00 | 15.35 | | C |
| ANISOU | 917 | CE1 | PHE | A | 119 | 1532 | 2667 | 1632 | −786 | 222 | −528 | C |
| ATOM | 918 | CE2 | PHE | A | 119 | −1.393 | 7.917 | 26.956 | 1.00 | 14.69 | | C |
| ANISOU | 918 | CE2 | PHE | A | 119 | 1867 | 2166 | 1549 | −593 | 466 | −574 | C |
| ATOM | 919 | CZ | PHE | A | 119 | −2.078 | 7.643 | 25.798 | 1.00 | 15.71 | | C |
| ANISOU | 919 | CZ | PHE | A | 119 | 1738 | 2343 | 1886 | −611 | 202 | −400 | C |
| ATOM | 920 | N | VAL | A | 120 | 3.052 | 12.327 | 24.235 | 1.00 | 7.67 | | N |
| ANISOU | 920 | N | VAL | A | 120 | 806 | 1213 | 896 | −25 | −69 | −243 | N |
| ATOM | 921 | CA | VAL | A | 120 | 3.641 | 13.630 | 23.941 | 1.00 | 7.64 | | C |
| ANISOU | 921 | CA | VAL | A | 120 | 980 | 1115 | 808 | 71 | −204 | −229 | C |
| ATOM | 922 | C | VAL | A | 120 | 2.584 | 14.465 | 23.238 | 1.00 | 8.07 | | C |
| ANISOU | 922 | C | VAL | A | 120 | 859 | 1185 | 1022 | −3 | −363 | −284 | C |
| ATOM | 923 | O | VAL | A | 120 | 1.927 | 13.919 | 22.331 | 1.00 | 9.41 | | O |
| ANISOU | 923 | O | VAL | A | 120 | 949 | 1395 | 1232 | 112 | −456 | −553 | O |
| ATOM | 924 | CB | VAL | A | 120 | 4.946 | 13.515 | 23.131 | 1.00 | 9.17 | | C |
| ANISOU | 924 | CB | VAL | A | 120 | 738 | 1240 | 1508 | −117 | −121 | −162 | C |
| ATOM | 925 | CG1 | VAL | A | 120 | 5.431 | 14.909 | 22.760 | 1.00 | 9.97 | | C |
| ANISOU | 925 | CG1 | VAL | A | 120 | 1088 | 1067 | 1633 | −167 | −48 | −329 | C |
| ATOM | 926 | CG2 | VAL | A | 120 | 5.998 | 12.714 | 23.866 | 1.00 | 11.52 | | C |
| ANISOU | 926 | CG2 | VAL | A | 120 | 937 | 1482 | 1958 | 111 | −6 | 106 | C |
| ATOM | 927 | N | ILE | A | 121 | 2.407 | 15.710 | 23.626 | 1.00 | 7.90 | | N |
| ANISOU | 927 | N | ILE | A | 121 | 776 | 1112 | 1115 | 18 | −270 | −249 | N |
| ATOM | 928 | CA | ILE | A | 121 | 1.353 | 16.562 | 23.073 | 1.00 | 7.58 | | C |
| ANISOU | 928 | CA | ILE | A | 121 | 768 | 1093 | 1020 | −101 | −333 | −258 | C |
| ATOM | 929 | C | ILE | A | 121 | 1.948 | 17.639 | 22.188 | 1.00 | 8.62 | | C |
| ANISOU | 929 | C | ILE | A | 121 | 924 | 1189 | 1162 | −71 | −347 | −154 | C |
| ATOM | 930 | O | ILE | A | 121 | 2.905 | 18.304 | 22.589 | 1.00 | 8.22 | | O |
| ANISOU | 930 | O | ILE | A | 121 | 917 | 1223 | 982 | −198 | −185 | −36 | O |
| ATOM | 931 | CB | ILE | A | 121 | 0.528 | 17.180 | 24.192 | 1.00 | 8.73 | | C |
| ANISOU | 931 | CB | ILE | A | 121 | 953 | 1051 | 1315 | −44 | −185 | −416 | C |
| ATOM | 932 | CG1 | ILE | A | 121 | 0.100 | 16.121 | 25.204 | 1.00 | 10.95 | | C |
| ANISOU | 932 | CG1 | ILE | A | 121 | 1644 | 1219 | 1299 | −286 | 306 | −584 | C |
| ATOM | 933 | CG2 | ILE | A | 121 | −0.636 | 17.978 | 23.618 | 1.00 | 10.74 | | C |
| ANISOU | 933 | CG2 | ILE | A | 121 | 757 | 1791 | 1532 | 79 | −351 | −673 | C |
| ATOM | 934 | CD1 | ILE | A | 121 | −0.796 | 15.045 | 24.687 | 1.00 | 12.85 | | C |
| ANISOU | 934 | CD1 | ILE | A | 121 | 1410 | 1197 | 2274 | −252 | 100 | −566 | C |
| ATOM | 935 | N | LEU | A | 122 | 1.422 | 17.748 | 20.970 | 1.00 | 7.55 | | N |
| ANISOU | 935 | N | LEU | A | 122 | 658 | 1166 | 1045 | 50 | −197 | −215 | N |
| ATOM | 936 | CA | LEU | A | 122 | 1.891 | 18.702 | 20.000 | 1.00 | 7.06 | | C |
| ANISOU | 936 | CA | LEU | A | 122 | 629 | 992 | 1059 | −96 | −127 | −339 | C |
| ATOM | 937 | C | LEU | A | 122 | 0.816 | 19.702 | 19.644 | 1.00 | 7.59 | | C |
| ANISOU | 937 | C | LEU | A | 122 | 608 | 1081 | 1195 | −114 | −177 | −136 | C |
| ATOM | 938 | O | LEU | A | 122 | −0.258 | 19.279 | 19.199 | 1.00 | 9.44 | | O |
| ANISOU | 938 | O | LEU | A | 122 | 595 | 1629 | 1362 | −178 | −201 | −462 | O |
| ATOM | 939 | CB | LEU | A | 122 | 2.326 | 18.036 | 18.686 | 1.00 | 9.83 | | C |
| ANISOU | 939 | CB | LEU | A | 122 | 874 | 1607 | 1253 | 18 | 129 | −438 | C |
| ATOM | 940 | CG | LEU | A | 122 | 3.286 | 16.840 | 18.806 | 1.00 | 17.71 | | C |
| ANISOU | 940 | CG | LEU | A | 122 | 2055 | 3151 | 1524 | 1509 | −176 | −950 | C |
| ATOM | 941 | CD1 | LEU | A | 122 | 3.686 | 16.437 | 17.393 | 1.00 | 17.31 | | C |
| ANISOU | 941 | CD1 | LEU | A | 122 | 2298 | 3033 | 1245 | 1691 | −33 | −258 | C |
| ATOM | 942 | CD2 | LEU | A | 122 | 4.451 | 17.116 | 19.676 | 1.00 | 24.11 | | C |
| ANISOU | 942 | CD2 | LEU | A | 122 | 1611 | 5909 | 1639 | 1687 | −155 | −766 | C |
| ATOM | 943 | N | GLY | A | 123 | 1.101 | 20.973 | 19.826 | 1.00 | 8.20 | | N |
| ANISOU | 943 | N | GLY | A | 123 | 878 | 1065 | 1173 | −3 | −332 | −148 | N |
| ATOM | 944 | CA | GLY | A | 123 | 0.251 | 22.078 | 19.378 | 1.00 | 7.49 | | C |
| ANISOU | 944 | CA | GLY | A | 123 | 773 | 1148 | 926 | −34 | −91 | 16 | C |
| ATOM | 945 | C | GLY | A | 123 | 0.886 | 22.652 | 18.123 | 1.00 | 7.01 | | C |
| ANISOU | 945 | C | GLY | A | 123 | 496 | 1393 | 775 | 39 | 13 | −118 | C |
| ATOM | 946 | O | GLY | A | 123 | 1.881 | 23.389 | 18.188 | 1.00 | 8.34 | | O |
| ANISOU | 946 | O | GLY | A | 123 | 793 | 1366 | 1012 | −214 | −85 | −86 | O |
| ATOM | 947 | N | ASN | A | 124 | 0.377 | 22.244 | 16.977 | 1.00 | 7.38 | | N |
| ANISOU | 947 | N | ASN | A | 124 | 684 | 1233 | 888 | −12 | −173 | −54 | N |
| ATOM | 948 | CA | ASN | A | 124 | 0.979 | 22.609 | 15.711 | 1.00 | 8.02 | | C |
| ANISOU | 948 | CA | ASN | A | 124 | 1065 | 1290 | 692 | −98 | −285 | −14 | C |
| ATOM | 949 | C | ASN | A | 124 | 0.455 | 23.886 | 15.078 | 1.00 | 7.85 | | C |
| ANISOU | 949 | C | ASN | A | 124 | 804 | 1267 | 912 | −194 | −184 | −3 | C |
| ATOM | 950 | O | ASN | A | 124 | −0.556 | 24.427 | 15.526 | 1.00 | 8.67 | | O |
| ANISOU | 950 | O | ASN | A | 124 | 767 | 1291 | 1237 | −176 | −91 | −14 | O |
| ATOM | 951 | CB | ASN | A | 124 | 0.800 | 21.415 | 14.740 | 1.00 | 8.43 | | C |
| ANISOU | 951 | CB | ASN | A | 124 | 895 | 1358 | 948 | −48 | −203 | −158 | C |
| ATOM | 952 | CG | ASN | A | 124 | 1.651 | 21.500 | 13.516 | 1.00 | 7.65 | | C |
| ANISOU | 952 | CG | ASN | A | 124 | 598 | 1259 | 1051 | 155 | −194 | −244 | C |
| ATOM | 953 | OD1 | ASN | A | 124 | 2.829 | 21.831 | 13.587 | 1.00 | 8.78 | | O |
| ANISOU | 953 | OD1 | ASN | A | 124 | 667 | 1495 | 1174 | 124 | −286 | −62 | O |
| ATOM | 954 | ND2 | ASN | A | 124 | 1.053 | 21.208 | 12.370 | 1.00 | 8.83 | | N |
| ANISOU | 954 | ND2 | ASN | A | 124 | 637 | 1725 | 995 | 22 | −101 | −169 | N |
| ATOM | 955 | N | LYS | A | 125 | 1.138 | 24.378 | 14.082 | 1.00 | 8.79 | | N |
| ANISOU | 955 | N | LYS | A | 125 | 1056 | 1244 | 1042 | −141 | −24 | 167 | N |
| ATOM | 956 | CA | LYS | A | 125 | 0.832 | 25.560 | 13.308 | 1.00 | 8.56 | | C |
| ANISOU | 956 | CA | LYS | A | 125 | 964 | 1248 | 1041 | 49 | −236 | 132 | C |
| ATOM | 957 | C | LYS | A | 125 | 1.093 | 26.840 | 14.053 | 1.00 | 9.93 | | C |
| ANISOU | 957 | C | LYS | A | 125 | 958 | 1281 | 1533 | −56 | −258 | 50 | C |
| ATOM | 958 | O | LYS | A | 125 | 0.412 | 27.836 | 13.834 | 1.00 | 10.30 | | O |
| ANISOU | 958 | O | LYS | A | 125 | 1318 | 1275 | 1320 | 148 | −127 | 44 | O |
| ATOM | 959 | CB | LYS | A | 125 | −0.624 | 25.535 | 12.741 | 1.00 | 10.04 | | C |
| ANISOU | 959 | CB | LYS | A | 125 | 826 | 1376 | 1613 | −144 | −160 | 122 | C |
| ATOM | 960 | CG | LYS | A | 125 | −0.977 | 24.241 | 12.047 | 1.00 | 9.53 | | C |
| ANISOU | 960 | CG | LYS | A | 125 | 919 | 1558 | 1145 | −1 | −179 | 32 | C |
| ATOM | 961 | CD | LYS | A | 125 | −2.218 | 24.412 | 11.172 | 1.00 | 10.41 | | C |
| ANISOU | 961 | CD | LYS | A | 125 | 1154 | 1266 | 1535 | −115 | −441 | 308 | C |
| ATOM | 962 | CE | LYS | A | 125 | −2.653 | 23.096 | 10.555 | 1.00 | 9.62 | | C |
| ANISOU | 962 | CE | LYS | A | 125 | 952 | 1540 | 1165 | −189 | −107 | 133 | C |
| ATOM | 963 | NZ | LYS | A | 125 | −3.788 | 23.284 | 9.606 | 1.00 | 11.40 | | N |
| ANISOU | 963 | NZ | LYS | A | 125 | 1385 | 1547 | 1399 | −202 | −476 | 292 | N |
| ATOM | 964 | N | ILE | A | 126 | 2.160 | 26.863 | 14.890 | 1.00 | 10.17 | | N |
| ANISOU | 964 | N | ILE | A | 126 | 1047 | 1485 | 1331 | −63 | −254 | −30 | N |
| ATOM | 965 | CA | ILE | A | 126 | 2.351 | 28.079 | 15.677 | 1.00 | 12.24 | | C |
| ANISOU | 965 | CA | ILE | A | 126 | 1724 | 1567 | 1361 | −407 | −180 | −35 | C |
| ATOM | 966 | C | ILE | A | 126 | 2.871 | 29.239 | 14.838 | 1.00 | 13.46 | | C |
| ANISOU | 966 | C | ILE | A | 126 | 1926 | 1528 | 1659 | −188 | 59 | 130 | C |
| ATOM | 967 | O | ILE | A | 126 | 2.982 | 30.355 | 15.350 | 1.00 | 13.14 | | O |
| ANISOU | 967 | O | ILE | A | 126 | 1884 | 1563 | 1545 | −273 | −33 | 177 | O |
| ATOM | 968 | CB | ILE | A | 126 | 3.309 | 27.846 | 16.840 | 1.00 | 12.77 | | C |
| ANISOU | 968 | CB | ILE | A | 126 | 1809 | 1804 | 1240 | −366 | −198 | −160 | C |
| ATOM | 969 | CG1 | ILE | A | 126 | 4.746 | 27.519 | 16.423 | 1.00 | 14.98 | | C |
| ANISOU | 969 | CG1 | ILE | A | 126 | 1886 | 1528 | 2279 | 2 | −188 | 12 | C |
| ATOM | 970 | CG2 | ILE | A | 126 | 2.679 | 26.739 | 17.620 | 1.00 | 16.53 | | C |
| ANISOU | 970 | CG2 | ILE | A | 126 | 2083 | 2147 | 2050 | −136 | −238 | 580 | C |
| ATOM | 971 | CD1 | ILE | A | 126 | 5.654 | 27.702 | 17.641 | 1.00 | 25.52 | | C |
| ANISOU | 971 | CD1 | ILE | A | 126 | 2487 | 3125 | 4083 | −513 | −1672 | 633 | C |
| ATOM | 972 | N | ASP | A | 127 | 3.173 | 28.954 | 13.563 | 1.00 | 12.01 | | N |
| ANISOU | 972 | N | ASP | A | 127 | 1414 | 1491 | 1660 | −273 | 12 | 139 | N |
| ATOM | 973 | CA | ASP | A | 127 | 3.481 | 30.065 | 12.656 | 1.00 | 12.71 | | C |
| ANISOU | 973 | CA | ASP | A | 127 | 1631 | 1512 | 1684 | −307 | −33 | 169 | C |
| ATOM | 974 | C | ASP | A | 127 | 2.261 | 30.919 | 12.344 | 1.00 | 13.29 | | C |
| ANISOU | 974 | C | ASP | A | 127 | 1842 | 1214 | 1994 | −159 | 46 | −9 | C |
| ATOM | 975 | O | ASP | A | 127 | 2.418 | 32.009 | 11.742 | 1.00 | 16.55 | | O |
| ANISOU | 975 | O | ASP | A | 127 | 2295 | 1609 | 2383 | −39 | 314 | 486 | O |
| ATOM | 976 | CB | ASP | A | 127 | 4.091 | 29.509 | 11.357 | 1.00 | 13.00 | | C |
| ANISOU | 976 | CB | ASP | A | 127 | 1226 | 1839 | 1875 | −326 | 176 | 149 | C |
| ATOM | 977 | CG | ASP | A | 127 | 3.115 | 28.531 | 10.716 | 1.00 | 12.39 | | C |
| ANISOU | 977 | CG | ASP | A | 127 | 1321 | 1494 | 1892 | 5 | −96 | 42 | C |
| ATOM | 978 | OD1 | ASP | A | 127 | 3.082 | 27.372 | 11.194 | 1.00 | 12.58 | | O |
| ANISOU | 978 | OD1 | ASP | A | 127 | 1354 | 1828 | 1599 | −265 | −145 | 309 | O |
| ATOM | 979 | OD2 | ASP | A | 127 | 2.401 | 28.909 | 9.761 | 1.00 | 13.26 | | O |
| ANISOU | 979 | OD2 | ASP | A | 127 | 1601 | 1835 | 1600 | −42 | 0 | 227 | O |
| ATOM | 980 | N | ILE | A | 128 | 1.048 | 30.479 | 12.720 | 1.00 | 13.29 | | N |
| ANISOU | 980 | N | ILE | A | 128 | 1734 | 1436 | 1882 | 95 | 42 | 316 | N |
| ATOM | 981 | CA | ILE | A | 128 | −0.172 | 31.249 | 12.460 | 1.00 | 16.96 | | C |
| ANISOU | 981 | CA | ILE | A | 128 | 1995 | 1833 | 2615 | 494 | −58 | 59 | C |
| ATOM | 982 | C | ILE | A | 128 | −0.408 | 32.145 | 13.675 | 1.00 | 18.04 | | C |
| ANISOU | 982 | C | ILE | A | 128 | 2243 | 1666 | 2946 | 137 | 537 | −61 | C |
| ATOM | 983 | O | ILE | A | 128 | −0.518 | 31.639 | 14.800 | 1.00 | 19.05 | | O |
| ANISOU | 983 | O | ILE | A | 128 | 2466 | 1959 | 2811 | 392 | 504 | −118 | O |
| ATOM | 984 | CB | ILE | A | 128 | −1.369 | 30.332 | 12.205 | 1.00 | 15.58 | | C |
| ANISOU | 984 | CB | ILE | A | 128 | 1836 | 2063 | 2022 | 635 | −334 | 218 | C |
| ATOM | 985 | CG1 | ILE | A | 128 | −1.098 | 29.433 | 10.985 | 1.00 | 15.47 | | C |
| ANISOU | 985 | CG1 | ILE | A | 128 | 1983 | 2005 | 1890 | 501 | −180 | 311 | C |
| ATOM | 986 | CG2 | ILE | A | 128 | −2.666 | 31.104 | 12.083 | 1.00 | 16.40 | | C |
| ANISOU | 986 | CG2 | ILE | A | 128 | 1981 | 2018 | 2233 | 763 | −155 | 648 | C |
| ATOM | 987 | CD1 | ILE | A | 128 | −2.189 | 28.427 | 10.736 | 1.00 | 17.66 | | C |
| ANISOU | 987 | CD1 | ILE | A | 128 | 2112 | 2077 | 2521 | 534 | −572 | 93 | C |
| ATOM | 988 | N | SER | A | 129 | −0.482 | 33.449 | 13.498 | 1.00 | 21.66 | | N |
| ANISOU | 988 | N | SER | A | 129 | 2670 | 1664 | 3896 | 154 | 900 | 51 | N |
| ATOM | 989 | CA | SER | A | 129 | −0.568 | 34.395 | 14.616 | 1.00 | 24.12 | | C |
| ANISOU | 989 | CA | SER | A | 129 | 3132 | 1596 | 4436 | 437 | 1203 | −204 | C |
| ATOM | 990 | C | SER | A | 129 | −1.932 | 34.324 | 15.309 | 1.00 | 22.29 | | C |
| ANISOU | 990 | C | SER | A | 129 | 2926 | 1861 | 3680 | 475 | 777 | 98 | C |
| ATOM | 991 | O | SER | A | 129 | −2.046 | 34.461 | 16.521 | 1.00 | 24.07 | | O |
| ANISOU | 991 | O | SER | A | 129 | 2606 | 2689 | 3852 | −92 | 612 | −1010 | O |
| ATOM | 992 | CB | SER | A | 129 | −0.294 | 35.842 | 14.257 | 1.00 | 30.02 | | C |
| ANISOU | 992 | CB | SER | A | 129 | 4595 | 1880 | 4930 | −488 | 1317 | −189 | C |
| ATOM | 993 | OG | SER | A | 129 | −0.215 | 36.240 | 12.909 | 1.00 | 50.01 | | O |
| ANISOU | 993 | OG | SER | A | 129 | 10275 | 2941 | 5785 | 695 | −1557 | 1748 | O |
| ATOM | 994 | N | GLU | A | 130 | −2.972 | 34.105 | 14.515 | 1.00 | 20.12 | | N |
| ANISOU | 994 | N | GLU | A | 130 | 3159 | 1643 | 2841 | 418 | 854 | 786 | N |
| ATOM | 995 | CA | GLU | A | 130 | −4.336 | 34.114 | 15.052 | 1.00 | 21.00 | | C |
| ANISOU | 995 | CA | GLU | A | 130 | 2918 | 2215 | 2847 | 539 | 556 | 1304 | C |
| ATOM | 996 | C | GLU | A | 130 | −4.641 | 32.753 | 15.671 | 1.00 | 17.04 | | C |
| ANISOU | 996 | C | GLU | A | 130 | 2581 | 1797 | 2096 | 377 | 334 | 611 | C |
| ATOM | 997 | O | GLU | A | 130 | −4.997 | 31.844 | 14.959 | 1.00 | 20.22 | | O |
| ANISOU | 997 | O | GLU | A | 130 | 3076 | 2230 | 2375 | 720 | −75 | 114 | O |
| ATOM | 998 | CB | GLU | A | 130 | −5.326 | 34.450 | 13.943 | 1.00 | 27.68 | | C |
| ANISOU | 998 | CB | GLU | A | 130 | 3790 | 3027 | 3699 | 603 | −245 | 1781 | C |
| ATOM | 999 | CG | GLU | A | 130 | −4.812 | 35.414 | 12.909 | 1.00 | 35.94 | | C |
| ANISOU | 999 | CG | GLU | A | 130 | 5458 | 5843 | 2356 | −441 | −68 | 1961 | C |
| ATOM | 1000 | CD | GLU | A | 130 | −4.011 | 34.900 | 11.727 | 1.00 | 45.78 | | C |
| ANISOU | 1000 | CD | GLU | A | 130 | 5137 | 8863 | 3394 | −1144 | 234 | 254 | C |
| ATOM | 1001 | OE1 | GLU | A | 130 | −4.590 | 34.473 | 10.697 | 1.00 | 66.85 | | O |
| ANISOU | 1001 | OE1 | GLU | A | 130 | 6051 | 13602 | 5746 | −2790 | 782 | −3723 | O |
| ATOM | 1002 | OE2 | GLU | A | 130 | −2.747 | 34.914 | 11.741 | 1.00 | 36.72 | | O |
| ANISOU | 1002 | OE2 | GLU | A | 130 | 5124 | 4999 | 3827 | −174 | 6 | 2332 | O |
| ATOM | 1003 | N | ARG | A | 131 | −4.528 | 32.722 | 16.993 | 1.00 | 13.93 | | N |
| ANISOU | 1003 | N | ARG | A | 131 | 1661 | 1557 | 2074 | 267 | 279 | 566 | N |
| ATOM | 1004 | CA | ARG | A | 131 | −4.728 | 31.438 | 17.677 | 1.00 | 14.10 | | C |
| ANISOU | 1004 | CA | ARG | A | 131 | 1884 | 1528 | 1947 | −189 | 160 | 422 | C |
| ATOM | 1005 | C | ARG | A | 131 | −6.104 | 31.312 | 18.286 | 1.00 | 14.25 | | C |
| ANISOU | 1005 | C | ARG | A | 131 | 1924 | 1417 | 2075 | 1 | 230 | 419 | C |
| ATOM | 1006 | O | ARG | A | 131 | −6.700 | 32.335 | 18.618 | 1.00 | 17.99 | | O |
| ANISOU | 1006 | O | ARG | A | 131 | 1914 | 1418 | 3503 | −137 | 402 | 155 | O |
| ATOM | 1007 | CB | ARG | A | 131 | −3.701 | 31.314 | 18.800 | 1.00 | 16.10 | | C |
| ANISOU | 1007 | CB | ARG | A | 131 | 1987 | 2107 | 2024 | 129 | 177 | 776 | C |
| ATOM | 1008 | CG | ARG | A | 131 | −2.271 | 31.250 | 18.284 | 1.00 | 17.78 | | C |
| ANISOU | 1008 | CG | ARG | A | 131 | 1910 | 2679 | 2166 | 4 | 171 | 596 | C |
| ATOM | 1009 | CD | ARG | A | 131 | −1.307 | 31.294 | 19.436 | 1.00 | 17.05 | | C |
| ANISOU | 1009 | CD | ARG | A | 131 | 2108 | 1970 | 2401 | −309 | −50 | 662 | C |
| ATOM | 1010 | NE | ARG | A | 131 | −1.342 | 30.105 | 20.260 | 1.00 | 16.20 | | N |
| ANISOU | 1010 | NE | ARG | A | 131 | 1740 | 1770 | 2647 | −103 | 7 | 650 | N |
| ATOM | 1011 | CZ | ARG | A | 131 | −0.346 | 29.327 | 20.664 | 1.00 | 14.72 | | C |
| ANISOU | 1011 | CZ | ARG | A | 131 | 1688 | 1602 | 2302 | −34 | −201 | −45 | C |
| ATOM | 1012 | NH1 | ARG | A | 131 | 0.916 | 29.534 | 20.339 | 1.00 | 19.05 | | N |
| ANISOU | 1012 | NH1 | ARG | A | 131 | 1839 | 2070 | 3329 | 102 | 218 | 237 | N |
| ATOM | 1013 | NH2 | ARG | A | 131 | −0.626 | 28.288 | 21.422 | 1.00 | 13.55 | | N |
| ANISOU | 1013 | NH2 | ARG | A | 131 | 1534 | 1581 | 2034 | 346 | −123 | −28 | N |
| ATOM | 1014 | N | GLN | A | 132 | −6.555 | 30.080 | 18.457 | 1.00 | 12.79 | | N |
| ANISOU | 1014 | N | GLN | A | 132 | 1256 | 1389 | 2216 | 82 | 86 | 241 | N |
| ATOM | 1015 | CA | GLN | A | 132 | −7.745 | 29.775 | 19.244 | 1.00 | 12.34 | | C |
| ANISOU | 1015 | CA | GLN | A | 132 | 1167 | 1348 | 2174 | 167 | −16 | 257 | C |
| ATOM | 1016 | C | GLN | A | 132 | −7.431 | 29.048 | 20.547 | 1.00 | 12.14 | | C |
| ANISOU | 1016 | C | GLN | A | 132 | 1229 | 1444 | 1941 | 491 | 197 | 96 | C |
| ATOM | 1017 | O | GLN | A | 132 | −8.314 | 28.874 | 21.394 | 1.00 | 14.34 | | O |
| ANISOU | 1017 | O | GLN | A | 132 | 1082 | 2210 | 2157 | 476 | 180 | 315 | O |
| ATOM | 1018 | CB | GLN | A | 132 | −8.680 | 28.898 | 18.439 | 1.00 | 14.09 | | C |
| ANISOU | 1018 | CB | GLN | A | 132 | 1331 | 1351 | 2673 | 8 | −465 | 569 | C |
| ATOM | 1019 | CG | GLN | A | 132 | −9.311 | 29.553 | 17.253 | 1.00 | 14.22 | | C |
| ANISOU | 1019 | CG | GLN | A | 132 | 2074 | 1489 | 1842 | 172 | −206 | 151 | C |
| ATOM | 1020 | CD | GLN | A | 132 | −10.378 | 28.735 | 16.554 | 1.00 | 14.06 | | C |
| ANISOU | 1020 | CD | GLN | A | 132 | 1360 | 1720 | 2263 | 490 | −197 | −46 | C |
| ATOM | 1021 | OE1 | GLN | A | 132 | −10.671 | 27.573 | 16.836 | 1.00 | 14.32 | | O |
| ANISOU | 1021 | OE1 | GLN | A | 132 | 1128 | 1994 | 2319 | 106 | −173 | 159 | O |
| ATOM | 1022 | NE2 | GLN | A | 132 | −10.989 | 29.421 | 15.594 | 1.00 | 19.18 | | N |
| ANISOU | 1022 | NE2 | GLN | A | 132 | 2106 | 2559 | 2621 | −378 | −658 | 802 | N |
| ATOM | 1023 | N | VAL | A | 133 | −6.217 | 28.631 | 20.743 | 1.00 | 10.92 | | N |
| ANISOU | 1023 | N | VAL | A | 133 | 1053 | 1486 | 1609 | 141 | −56 | −116 | N |
| ATOM | 1024 | CA | VAL | A | 133 | −5.737 | 27.942 | 21.933 | 1.00 | 12.08 | | C |
| ANISOU | 1024 | CA | VAL | A | 133 | 1310 | 1360 | 1919 | −94 | −236 | 128 | C |
| ATOM | 1025 | C | VAL | A | 133 | −4.461 | 28.659 | 22.414 | 1.00 | 10.87 | | C |
| ANISOU | 1025 | C | VAL | A | 133 | 1039 | 1290 | 1800 | 187 | −206 | 101 | C |
| ATOM | 1026 | O | VAL | A | 133 | −3.529 | 28.746 | 21.632 | 1.00 | 12.39 | | O |
| ANISOU | 1026 | O | VAL | A | 133 | 1254 | 1473 | 1982 | −36 | −54 | −265 | O |
| ATOM | 1027 | CB | VAL | A | 133 | −5.411 | 26.477 | 21.665 | 1.00 | 11.63 | | C |
| ANISOU | 1027 | CB | VAL | A | 133 | 1161 | 1468 | 1791 | 54 | −180 | 35 | C |
| ATOM | 1028 | CG1 | VAL | A | 133 | −5.074 | 25.764 | 22.961 | 1.00 | 11.24 | | C |
| ANISOU | 1028 | CG1 | VAL | A | 133 | 1239 | 1507 | 1526 | 275 | 221 | 29 | C |
| ATOM | 1029 | CG2 | VAL | A | 133 | −6.525 | 25.740 | 20.951 | 1.00 | 13.56 | | C |
| ANISOU | 1029 | CG2 | VAL | A | 133 | 1602 | 1378 | 2173 | −98 | −437 | 137 | C |
| ATOM | 1030 | N | SER | A | 134 | −4.438 | 29.149 | 23.662 | 1.00 | 12.23 | | N |
| ANISOU | 1030 | N | SER | A | 134 | 1490 | 1342 | 1814 | 66 | −191 | 38 | N |
| ATOM | 1031 | CA | SER | A | 134 | −3.230 | 29.785 | 24.162 | 1.00 | 12.74 | | C |
| ANISOU | 1031 | CA | SER | A | 134 | 1572 | 1375 | 1895 | 8 | −262 | −28 | C |
| ATOM | 1032 | C | SER | A | 134 | −2.233 | 28.745 | 24.654 | 1.00 | 11.31 | | C |
| ANISOU | 1032 | C | SER | A | 134 | 1268 | 1272 | 1759 | −134 | 38 | 0 | C |
| ATOM | 1033 | O | SER | A | 134 | −2.570 | 27.615 | 25.003 | 1.00 | 10.37 | | O |
| ANISOU | 1033 | O | SER | A | 134 | 1295 | 1308 | 1338 | −96 | −83 | −43 | O |
| ATOM | 1034 | CB | SER | A | 134 | −3.527 | 30.740 | 25.317 | 1.00 | 14.08 | | C |
| ANISOU | 1034 | CB | SER | A | 134 | 1397 | 1573 | 2378 | −31 | 1 | −331 | C |
| ATOM | 1035 | OG | SER | A | 134 | −3.999 | 30.004 | 26.432 | 1.00 | 15.16 | | O |
| ANISOU | 1035 | OG | SER | A | 134 | 1891 | 1915 | 1954 | 267 | −213 | −155 | O |
| ATOM | 1036 | N | THR | A | 135 | −0.983 | 29.188 | 24.708 | 1.00 | 11.57 | | N |
| ANISOU | 1036 | N | THR | A | 135 | 1396 | 1412 | 1590 | −285 | −300 | −215 | N |
| ATOM | 1037 | CA | THR | A | 135 | 0.059 | 28.316 | 25.258 | 1.00 | 11.94 | | C |
| ANISOU | 1037 | CA | THR | A | 135 | 1396 | 1458 | 1682 | −177 | −217 | −265 | C |
| ATOM | 1038 | C | THR | A | 135 | −0.324 | 27.869 | 26.652 | 1.00 | 11.99 | | C |
| ANISOU | 1038 | C | THR | A | 135 | 1370 | 1541 | 1646 | 34 | −213 | −224 | C |
| ATOM | 1039 | O | THR | A | 135 | −0.238 | 26.704 | 27.019 | 1.00 | 11.59 | | O |
| ANISOU | 1039 | O | THR | A | 135 | 1382 | 1495 | 1527 | −39 | −304 | −327 | O |
| ATOM | 1040 | CB | THR | A | 135 | 1.410 | 29.024 | 25.238 | 1.00 | 14.01 | | C |
| ANISOU | 1040 | CB | THR | A | 135 | 1435 | 1828 | 2062 | −332 | −381 | 107 | C |
| ATOM | 1041 | OG1 | THR | A | 135 | 1.719 | 29.420 | 23.870 | 1.00 | 14.49 | | O |
| ANISOU | 1041 | OG1 | THR | A | 135 | 1659 | 1613 | 2235 | −216 | −110 | 165 | O |
| ATOM | 1042 | CG2 | THR | A | 135 | 2.525 | 28.138 | 25.692 | 1.00 | 13.14 | | C |
| ANISOU | 1042 | CG2 | THR | A | 135 | 1303 | 1736 | 1954 | −207 | 231 | 187 | C |
| ATOM | 1043 | N | GLU | A | 136 | −0.786 | 28.822 | 27.473 | 1.00 | 12.97 | | N |
| ANISOU | 1043 | N | GLU | A | 136 | 1641 | 1613 | 1675 | 254 | −204 | −207 | N |
| ATOM | 1044 | CA | GLU | A | 136 | −1.082 | 28.530 | 28.861 | 1.00 | 13.32 | | C |
| ANISOU | 1044 | CA | GLU | A | 136 | 1577 | 1902 | 1580 | 124 | −282 | −247 | C |
| ATOM | 1045 | C | GLU | A | 136 | −2.189 | 27.514 | 29.006 | 1.00 | 11.93 | | C |
| ANISOU | 1045 | C | GLU | A | 136 | 1383 | 1831 | 1319 | 241 | −172 | −508 | C |
| ATOM | 1046 | O | GLU | A | 136 | −2.155 | 26.618 | 29.851 | 1.00 | 12.22 | | O |
| ANISOU | 1046 | O | GLU | A | 136 | 1277 | 2204 | 1162 | 17 | −195 | −371 | O |
| ATOM | 1047 | CB | GLU | A | 136 | −1.427 | 29.859 | 29.605 | 1.00 | 24.10 | | C |
| ANISOU | 1047 | CB | GLU | A | 136 | 4814 | 2434 | 1910 | −536 | −476 | −1306 | C |
| ATOM | 1048 | CG | GLU | A | 136 | −0.120 | 30.601 | 29.856 | 1.00 | 40.55 | | C |
| ANISOU | 1048 | CG | GLU | A | 136 | 7090 | 3654 | 4663 | −2738 | −1534 | −720 | C |
| ATOM | 1049 | CD | GLU | A | 136 | −0.156 | 32.091 | 30.012 | 1.00 | 48.24 | | C |
| ANISOU | 1049 | CD | GLU | A | 136 | 7341 | 3606 | 7380 | −2334 | −2064 | −496 | C |
| ATOM | 1050 | OE1 | GLU | A | 136 | −0.658 | 32.624 | 31.031 | 1.00 | 72.79 | | O |
| ANISOU | 1050 | OE1 | GLU | A | 136 | 11391 | 6730 | 9536 | 698 | −2934 | −3775 | O |
| ATOM | 1051 | OE2 | GLU | A | 136 | 0.376 | 32.769 | 29.094 | 1.00 | 67.57 | | O |
| ANISOU | 1051 | OE2 | GLU | A | 136 | 10241 | 6019 | 9415 | −4918 | −4882 | 3137 | O |
| ATOM | 1052 | N | GLU | A | 137 | −3.196 | 27.619 | 28.148 | 1.00 | 12.02 | | N |
| ANISOU | 1052 | N | GLU | A | 137 | 1373 | 1739 | 1455 | 378 | −171 | −371 | N |
| ATOM | 1053 | CA | GLU | A | 137 | −4.325 | 26.662 | 28.203 | 1.00 | 12.72 | | C |
| ANISOU | 1053 | CA | GLU | A | 137 | 1211 | 2099 | 1523 | 305 | −176 | −480 | C |
| ATOM | 1054 | C | GLU | A | 137 | −3.818 | 25.285 | 27.808 | 1.00 | 11.09 | | C |
| ANISOU | 1054 | C | GLU | A | 137 | 494 | 2000 | 1722 | −24 | −165 | −446 | C |
| ATOM | 1055 | O | GLU | A | 137 | −4.217 | 24.252 | 28.407 | 1.00 | 11.71 | | O |
| ANISOU | 1055 | O | GLU | A | 137 | 873 | 2104 | 1472 | 73 | −3 | −321 | O |
| ATOM | 1056 | CB | GLU | A | 137 | −5.471 | 27.167 | 27.336 | 1.00 | 16.61 | | C |
| ANISOU | 1056 | CB | GLU | A | 137 | 1481 | 2383 | 2448 | 323 | −510 | 98 | C |
| ATOM | 1057 | CG | GLU | A | 137 | −6.157 | 26.418 | 26.298 | 1.00 | 27.50 | | C |
| ANISOU | 1057 | CG | GLU | A | 137 | 2871 | 3917 | 3660 | 215 | −2032 | −231 | C |
| ATOM | 1058 | CD | GLU | A | 137 | −7.216 | 27.219 | 25.537 | 1.00 | 31.77 | | C |
| ANISOU | 1058 | CD | GLU | A | 137 | 2652 | 4255 | 5166 | −251 | −2451 | 655 | C |
| ATOM | 1059 | OE1 | GLU | A | 137 | −6.994 | 28.397 | 25.190 | 1.00 | 30.62 | | O |
| ANISOU | 1059 | OE1 | GLU | A | 137 | 1351 | 4499 | 5784 | 343 | −580 | 1107 | O |
| ATOM | 1060 | OE2 | GLU | A | 137 | −8.252 | 26.566 | 25.325 | 1.00 | 37.76 | | O |
| ANISOU | 1060 | OE2 | GLU | A | 137 | 1614 | 4713 | 8021 | −25 | −1584 | 926 | O |
| ATOM | 1061 | N | ALA | A | 138 | −2.945 | 25.172 | 26.809 | 1.00 | 9.36 | | N |
| ANISOU | 1061 | N | ALA | A | 138 | 562 | 1432 | 1562 | −45 | −239 | −286 | N |
| ATOM | 1062 | CA | ALA | A | 138 | −2.445 | 23.870 | 26.404 | 1.00 | 8.34 | | C |
| ANISOU | 1062 | CA | ALA | A | 138 | 958 | 1187 | 1024 | −295 | −228 | −237 | C |
| ATOM | 1063 | C | ALA | A | 138 | −1.587 | 23.274 | 27.518 | 1.00 | 8.84 | | C |
| ANISOU | 1063 | C | ALA | A | 138 | 906 | 1342 | 1112 | −131 | −219 | −171 | C |
| ATOM | 1064 | O | ALA | A | 138 | −1.684 | 22.082 | 27.806 | 1.00 | 8.67 | | O |
| ANISOU | 1064 | O | ALA | A | 138 | 790 | 1394 | 1109 | −86 | −7 | −128 | O |
| ATOM | 1065 | CB | ALA | A | 138 | −1.638 | 24.004 | 25.142 | 1.00 | 10.44 | | C |
| ANISOU | 1065 | CB | ALA | A | 138 | 1138 | 1738 | 1091 | −10 | −111 | −149 | C |
| ATOM | 1066 | N | GLN | A | 139 | −0.732 | 24.103 | 28.145 | 1.00 | 8.27 | | N |
| ANISOU | 1066 | N | GLN | A | 139 | 723 | 1276 | 1145 | 69 | −128 | −366 | N |
| ATOM | 1067 | CA | GLN | A | 139 | 0.140 | 23.591 | 29.165 | 1.00 | 9.40 | | C |
| ANISOU | 1067 | CA | GLN | A | 139 | 882 | 1626 | 1065 | 67 | −234 | −306 | C |
| ATOM | 1068 | C | GLN | A | 139 | −0.648 | 23.092 | 30.370 | 1.00 | 10.61 | | C |
| ANISOU | 1068 | C | GLN | A | 139 | 847 | 2142 | 1044 | −107 | −273 | −269 | C |
| ATOM | 1069 | O | GLN | A | 139 | −0.334 | 22.064 | 30.956 | 1.00 | 10.31 | | O |
| ANISOU | 1069 | O | GLN | A | 139 | 792 | 2152 | 973 | −119 | −133 | −231 | O |
| ATOM | 1070 | CB | GLN | A | 139 | 1.120 | 24.680 | 29.622 | 1.00 | 10.28 | | C |
| ANISOU | 1070 | CB | GLN | A | 139 | 930 | 1804 | 1172 | −47 | −210 | −402 | C |
| ATOM | 1071 | CG | GLN | A | 139 | 2.135 | 25.050 | 28.571 | 1.00 | 11.62 | | C |
| ANISOU | 1071 | CG | GLN | A | 139 | 1064 | 2083 | 1268 | −87 | −146 | −127 | C |
| ATOM | 1072 | CD | GLN | A | 139 | 2.830 | 26.374 | 28.814 | 1.00 | 12.72 | | C |
| ANISOU | 1072 | CD | GLN | A | 139 | 1236 | 2437 | 1161 | −414 | −112 | −253 | C |
| ATOM | 1073 | OE1 | GLN | A | 139 | 2.187 | 27.340 | 29.267 | 1.00 | 15.50 | | O |
| ANISOU | 1073 | OE1 | GLN | A | 139 | 1483 | 2307 | 2098 | −385 | −261 | −543 | O |
| ATOM | 1074 | NE2 | GLN | A | 139 | 4.120 | 26.498 | 28.539 | 1.00 | 16.27 | | N |
| ANISOU | 1074 | NE2 | GLN | A | 139 | 1132 | 3277 | 1774 | −556 | −297 | −451 | N |
| ATOM | 1075 | N | ALA | A | 140 | −1.694 | 23.814 | 30.744 | 1.00 | 10.58 | | N |
| ANISOU | 1075 | N | ALA | A | 140 | 817 | 2039 | 1164 | −159 | −238 | −54 | N |
| ATOM | 1076 | CA | ALA | A | 140 | −2.548 | 23.420 | 31.849 | 1.00 | 10.97 | | C |
| ANISOU | 1076 | CA | ALA | A | 140 | 1090 | 2006 | 1072 | 272 | −98 | −59 | C |
| ATOM | 1077 | C | ALA | A | 140 | −3.209 | 22.072 | 31.570 | 1.00 | 10.11 | | C |
| ANISOU | 1077 | C | ALA | A | 140 | 697 | 2207 | 939 | 26 | 165 | 10 | C |
| ATOM | 1078 | O | ALA | A | 140 | −3.293 | 21.180 | 32.435 | 1.00 | 11.63 | | O |
| ANISOU | 1078 | O | ALA | A | 140 | 947 | 2259 | 1212 | 111 | −73 | 218 | O |
| ATOM | 1079 | CB | ALA | A | 140 | −3.597 | 24.469 | 32.167 | 1.00 | 13.27 | | C |
| ANISOU | 1079 | CB | ALA | A | 140 | 1534 | 2306 | 1201 | 723 | −214 | 30 | C |
| ATOM | 1080 | N | TRP | A | 141 | −3.700 | 21.854 | 30.341 | 1.00 | 11.38 | | N |
| ANISOU | 1080 | N | TRP | A | 141 | 1024 | 2228 | 1073 | −54 | −57 | 78 | N |
| ATOM | 1081 | CA | TRP | A | 141 | −4.319 | 20.575 | 29.996 | 1.00 | 11.39 | | C |
| ANISOU | 1081 | CA | TRP | A | 141 | 1039 | 2328 | 959 | −97 | −68 | 105 | C |
| ATOM | 1082 | C | TRP | A | 141 | −3.276 | 19.474 | 30.159 | 1.00 | 11.00 | | C |
| ANISOU | 1082 | C | TRP | A | 141 | 816 | 2063 | 1298 | −409 | −159 | 89 | C |
| ATOM | 1083 | O | TRP | A | 141 | −3.544 | 18.432 | 30.767 | 1.00 | 10.69 | | O |
| ANISOU | 1083 | O | TRP | A | 141 | 961 | 2105 | 996 | −330 | −126 | 50 | O |
| ATOM | 1084 | CB | TRP | A | 141 | −4.921 | 20.590 | 28.593 | 1.00 | 11.85 | | C |
| ANISOU | 1084 | CB | TRP | A | 141 | 1393 | 2196 | 915 | −441 | −135 | 354 | C |
| ATOM | 1085 | CG | TRP | A | 141 | −5.676 | 19.300 | 28.343 | 1.00 | 11.50 | | C |
| ANISOU | 1085 | CG | TRP | A | 141 | 906 | 2152 | 1311 | −274 | −436 | 518 | C |
| ATOM | 1086 | CD1 | TRP | A | 141 | −6.983 | 19.068 | 28.610 | 1.00 | 14.11 | | C |
| ANISOU | 1086 | CD1 | TRP | A | 141 | 848 | 2646 | 1867 | −202 | −460 | 506 | C |
| ATOM | 1087 | CD2 | TRP | A | 141 | −5.133 | 18.087 | 27.819 | 1.00 | 11.94 | | C |
| ANISOU | 1087 | CD2 | TRP | A | 141 | 958 | 2351 | 1228 | −539 | −335 | 88 | C |
| ATOM | 1088 | NE1 | TRP | A | 141 | −7.335 | 17.776 | 28.274 | 1.00 | 16.86 | | N |
| ANISOU | 1088 | NE1 | TRP | A | 141 | 990 | 3152 | 2262 | −770 | −300 | 6 | N |
| ATOM | 1089 | CE2 | TRP | A | 141 | −6.201 | 17.145 | 27.784 | 1.00 | 14.05 | | C |
| ANISOU | 1089 | CE2 | TRP | A | 141 | 1252 | 2663 | 1423 | −783 | −419 | −32 | C |
| ATOM | 1090 | CE3 | TRP | A | 141 | −3.851 | 17.725 | 27.393 | 1.00 | 12.11 | | C |
| ANISOU | 1090 | CE3 | TRP | A | 141 | 1191 | 2430 | 980 | −378 | −153 | 163 | C |
| ATOM | 1091 | CZ2 | TRP | A | 141 | −6.004 | 15.871 | 27.320 | 1.00 | 16.85 | | C |
| ANISOU | 1091 | CZ2 | TRP | A | 141 | 1618 | 2609 | 2178 | −769 | −815 | −154 | C |
| ATOM | 1092 | CZ3 | TRP | A | 141 | −3.690 | 16.446 | 26.934 | 1.00 | 14.41 | | C |
| ANISOU | 1092 | CZ3 | TRP | A | 141 | 1566 | 2493 | 1415 | −207 | −240 | 89 | C |
| ATOM | 1093 | CH2 | TRP | A | 141 | −4.740 | 15.536 | 26.898 | 1.00 | 17.08 | | C |
| ANISOU | 1093 | CH2 | TRP | A | 141 | 1931 | 2603 | 1956 | −447 | −616 | −206 | C |
| ATOM | 1094 | N | CYS | A | 142 | −2.065 | 19.683 | 29.656 | 1.00 | 10.16 | | N |
| ANISOU | 1094 | N | CYS | A | 142 | 747 | 2160 | 954 | −408 | −180 | −187 | N |
| ATOM | 1095 | CA | CYS | A | 142 | −1.031 | 18.692 | 29.771 | 1.00 | 10.78 | | C |
| ANISOU | 1095 | CA | CYS | A | 142 | 1001 | 1979 | 1116 | −343 | −146 | −131 | C |
| ATOM | 1096 | C | CYS | A | 142 | −0.682 | 18.376 | 31.219 | 1.00 | 10.69 | | C |
| ANISOU | 1096 | C | CYS | A | 142 | 1092 | 1937 | 1035 | −152 | −184 | −244 | C |
| ATOM | 1097 | O | CYS | A | 142 | −0.525 | 17.222 | 31.583 | 1.00 | 11.21 | | O |
| ANISOU | 1097 | O | CYS | A | 142 | 1176 | 1890 | 1195 | −443 | −267 | −119 | O |
| ATOM | 1098 | CB | CYS | A | 142 | 0.209 | 19.150 | 28.986 | 1.00 | 10.50 | | C |
| ANISOU | 1098 | CB | CYS | A | 142 | 981 | 1752 | 1258 | −58 | 60 | −170 | C |
| ATOM | 1099 | SG | CYS | A | 142 | −0.061 | 19.184 | 27.194 | 1.00 | 11.59 | | S |
| ANISOU | 1099 | SG | CYS | A | 142 | 1340 | 1825 | 1239 | −304 | 114 | −332 | S |
| ATOM | 1100 | N | ARG | A | 143 | −0.572 | 19.410 | 32.058 | 1.00 | 10.02 | | N |
| ANISOU | 1100 | N | ARG | A | 143 | 704 | 1901 | 1202 | 167 | −334 | −175 | N |
| ATOM | 1101 | CA | ARG | A | 143 | −0.235 | 19.187 | 33.460 | 1.00 | 10.63 | | C |
| ANISOU | 1101 | CA | ARG | A | 143 | 913 | 2076 | 1052 | −60 | −336 | −344 | C |
| ATOM | 1102 | C | ARG | A | 143 | −1.273 | 18.356 | 34.179 | 1.00 | 11.76 | | C |
| ANISOU | 1102 | C | ARG | A | 143 | 944 | 2240 | 1284 | 186 | −142 | −60 | C |
| ATOM | 1103 | O | ARG | A | 143 | −0.929 | 17.519 | 34.978 | 1.00 | 12.27 | | O |
| ANISOU | 1103 | O | ARG | A | 143 | 1109 | 2274 | 1278 | 115 | −238 | −90 | O |
| ATOM | 1104 | CB | ARG | A | 143 | −0.108 | 20.520 | 34.199 | 1.00 | 13.24 | | C |
| ANISOU | 1104 | CB | ARG | A | 143 | 1066 | 2248 | 1717 | 128 | −66 | −764 | C |
| ATOM | 1105 | CG | ARG | A | 143 | 1.114 | 21.362 | 33.912 | 1.00 | 16.18 | | C |
| ANISOU | 1105 | CG | ARG | A | 143 | 1334 | 2450 | 2364 | −296 | −63 | −1075 | C |
| ATOM | 1106 | CD | ARG | A | 143 | 1.337 | 22.366 | 35.064 | 1.00 | 13.34 | | C |
| ANISOU | 1106 | CD | ARG | A | 143 | 1545 | 1774 | 1750 | 220 | −469 | −440 | C |
| ATOM | 1107 | NE | ARG | A | 143 | 0.178 | 23.236 | 35.178 | 1.00 | 13.18 | | N |
| ANISOU | 1107 | NE | ARG | A | 143 | 1425 | 2131 | 1453 | 233 | −311 | −287 | N |
| ATOM | 1108 | CZ | ARG | A | 143 | −0.053 | 24.321 | 34.450 | 1.00 | 11.84 | | C |
| ANISOU | 1108 | CZ | ARG | A | 143 | 837 | 1803 | 1859 | 63 | −360 | −376 | C |
| ATOM | 1109 | NH1 | ARG | A | 143 | 0.807 | 24.684 | 33.519 | 1.00 | 12.58 | | N |
| ANISOU | 1109 | NH1 | ARG | A | 143 | 1043 | 2079 | 1659 | −222 | −531 | −261 | N |
| ATOM | 1110 | NH2 | ARG | A | 143 | −1.126 | 25.073 | 34.610 | 1.00 | 15.16 | | N |
| ANISOU | 1110 | NH2 | ARG | A | 143 | 1397 | 2394 | 1969 | 635 | −439 | −716 | N |
| ATOM | 1111 | N | ASP | A | 144 | −2.528 | 18.628 | 33.820 | 1.00 | 12.36 | | N |
| ANISOU | 1111 | N | ASP | A | 144 | 984 | 2793 | 921 | −55 | −163 | 376 | N |
| ATOM | 1112 | CA | ASP | A | 144 | −3.654 | 18.084 | 34.549 | 1.00 | 11.79 | | C |
| ANISOU | 1112 | CA | ASP | A | 144 | 1068 | 2678 | 733 | 75 | 89 | 102 | C |
| ATOM | 1113 | C | ASP | A | 144 | −4.174 | 16.761 | 34.006 | 1.00 | 14.35 | | C |
| ANISOU | 1113 | C | ASP | A | 144 | 1480 | 2654 | 1319 | −156 | 26 | 35 | C |
| ATOM | 1114 | O | ASP | A | 144 | −5.057 | 16.164 | 34.633 | 1.00 | 18.18 | | O |
| ANISOU | 1114 | O | ASP | A | 144 | 1733 | 3475 | 1699 | −687 | 142 | 11 | O |
| ATOM | 1115 | CB | ASP | A | 144 | −4.783 | 19.106 | 34.636 | 1.00 | 15.01 | | C |
| ANISOU | 1115 | CB | ASP | A | 144 | 1351 | 2700 | 1653 | 255 | 194 | 229 | C |
| ATOM | 1116 | CG | ASP | A | 144 | −4.463 | 20.348 | 35.444 | 1.00 | 16.12 | | C |
| ANISOU | 1116 | CG | ASP | A | 144 | 1326 | 2836 | 1963 | 317 | 305 | −43 | C |
| ATOM | 1117 | OD1 | ASP | A | 144 | −3.486 | 20.345 | 36.200 | 1.00 | 18.01 | | O |
| ANISOU | 1117 | OD1 | ASP | A | 144 | 1353 | 3444 | 2047 | 98 | 207 | −205 | O |
| ATOM | 1118 | OD2 | ASP | A | 144 | −5.250 | 21.310 | 35.320 | 1.00 | 17.39 | | O |
| ANISOU | 1118 | OD2 | ASP | A | 144 | 2070 | 2768 | 1769 | 528 | 341 | 148 | O |
| ATOM | 1119 | N | ASN | A | 145 | −3.635 | 16.316 | 32.873 | 1.00 | 13.26 | | N |
| ANISOU | 1119 | N | ASN | A | 145 | 1656 | 2295 | 1089 | 57 | −207 | 100 | N |
| ATOM | 1120 | CA | ASN | A | 145 | −4.133 | 15.075 | 32.248 | 1.00 | 13.23 | | C |
| ANISOU | 1120 | CA | ASN | A | 145 | 1122 | 2353 | 1552 | −135 | −45 | 63 | C |
| ATOM | 1121 | C | ASN | A | 145 | −3.035 | 14.048 | 31.990 | 1.00 | 12.77 | | C |
| ANISOU | 1121 | C | ASN | A | 145 | 864 | 2622 | 1367 | −284 | 80 | −463 | C |
| ATOM | 1122 | O | ASN | A | 145 | −3.079 | 13.284 | 31.053 | 1.00 | 19.05 | | O |
| ANISOU | 1122 | O | ASN | A | 145 | 1883 | 3527 | 1829 | −100 | −66 | −1071 | O |
| ATOM | 1123 | CB | ASN | A | 145 | −4.826 | 15.425 | 30.923 | 1.00 | 13.95 | | C |
| ANISOU | 1123 | CB | ASN | A | 145 | 1475 | 2252 | 1572 | −443 | −217 | −1 | C |
| ATOM | 1124 | CG | ASN | A | 145 | −6.132 | 16.138 | 31.190 | 1.00 | 14.84 | | C |
| ANISOU | 1124 | CG | ASN | A | 145 | 1443 | 2386 | 1810 | −295 | −126 | 947 | C |
| ATOM | 1125 | OD1 | ASN | A | 145 | −6.194 | 17.364 | 31.300 | 1.00 | 16.81 | | O |
| ANISOU | 1125 | OD1 | ASN | A | 145 | 1561 | 2454 | 2373 | −51 | −462 | 462 | O |
| ATOM | 1126 | ND2 | ASN | A | 145 | −7.098 | 15.260 | 31.290 | 1.00 | 18.58 | | N |
| ANISOU | 1126 | ND2 | ASN | A | 145 | 1432 | 2631 | 2997 | −354 | −149 | 1087 | N |
| ATOM | 1127 | N | GLY | A | 146 | −2.053 | 14.028 | 32.878 | 1.00 | 11.38 | | N |
| ANISOU | 1127 | N | GLY | A | 146 | 1330 | 1711 | 1282 | −112 | −106 | −3 | N |
| ATOM | 1128 | CA | GLY | A | 146 | −0.956 | 13.116 | 32.868 | 1.00 | 12.04 | | C |
| ANISOU | 1128 | CA | GLY | A | 146 | 1382 | 1612 | 1579 | −220 | −94 | −34 | C |
| ATOM | 1129 | C | GLY | A | 146 | 0.431 | 13.704 | 32.958 | 1.00 | 11.93 | | C |
| ANISOU | 1129 | C | GLY | A | 146 | 1269 | 1925 | 1338 | −160 | −212 | −125 | C |
| ATOM | 1130 | O | GLY | A | 146 | 1.373 | 12.913 | 33.000 | 1.00 | 13.76 | | O |
| ANISOU | 1130 | O | GLY | A | 146 | 1501 | 2089 | 1639 | 4 | −458 | 118 | O |
| ATOM | 1131 | N | ASP | A | 147 | 0.540 | 15.006 | 32.938 | 1.00 | 10.81 | | N |
| ANISOU | 1131 | N | ASP | A | 147 | 1041 | 1945 | 1122 | −263 | 62 | −37 | N |
| ATOM | 1132 | CA | ASP | A | 147 | 1.818 | 15.733 | 32.985 | 1.00 | 10.96 | | C |
| ANISOU | 1132 | CA | ASP | A | 147 | 860 | 2203 | 1103 | −192 | 53 | −168 | C |
| ATOM | 1133 | C | ASP | A | 147 | 2.714 | 15.350 | 31.830 | 1.00 | 9.81 | | C |
| ANISOU | 1133 | C | ASP | A | 147 | 837 | 1730 | 1161 | 89 | −64 | −362 | C |
| ATOM | 1134 | O | ASP | A | 147 | 3.895 | 15.078 | 31.984 | 1.00 | 15.00 | | O |
| ANISOU | 1134 | O | ASP | A | 147 | 977 | 3236 | 1484 | 480 | −181 | −213 | O |
| ATOM | 1135 | CB | ASP | A | 147 | 2.488 | 15.498 | 34.334 | 1.00 | 16.56 | | C |
| ANISOU | 1135 | CB | ASP | A | 147 | 1881 | 3339 | 1071 | −532 | −379 | −485 | C |
| ATOM | 1136 | CG | ASP | A | 147 | 3.616 | 16.495 | 34.538 | 1.00 | 23.43 | | C |
| ANISOU | 1136 | CG | ASP | A | 147 | 2480 | 4480 | 1945 | −1301 | −816 | −462 | C |
| ATOM | 1137 | OD1 | ASP | A | 147 | 3.522 | 17.599 | 33.957 | 1.00 | 20.61 | | O |
| ANISOU | 1137 | OD1 | ASP | A | 147 | 1867 | 4050 | 1915 | −1301 | −4 | −860 | O |
| ATOM | 1138 | OD2 | ASP | A | 147 | 4.566 | 16.167 | 35.289 | 1.00 | 24.33 | | O |
| ANISOU | 1138 | OD2 | ASP | A | 147 | 2148 | 4742 | 2355 | −603 | −806 | −1344 | O |
| ATOM | 1139 | N | TYR | A | 148 | 2.173 | 15.361 | 30.627 | 1.00 | 8.84 | | N |
| ANISOU | 1139 | N | TYR | A | 148 | 694 | 1515 | 1151 | −187 | −21 | −187 | N |
| ATOM | 1140 | CA | TYR | A | 148 | 2.892 | 15.014 | 29.430 | 1.00 | 8.61 | | C |
| ANISOU | 1140 | CA | TYR | A | 148 | 776 | 1426 | 1071 | −4 | −113 | −50 | C |
| ATOM | 1141 | C | TYR | A | 148 | 3.736 | 16.181 | 28.939 | 1.00 | 8.93 | | C |
| ANISOU | 1141 | C | TYR | A | 148 | 1123 | 1453 | 818 | −238 | 17 | −297 | C |
| ATOM | 1142 | O | TYR | A | 148 | 3.365 | 17.335 | 29.147 | 1.00 | 10.21 | | O |
| ANISOU | 1142 | O | TYR | A | 148 | 1197 | 1408 | 1275 | 6 | −172 | −88 | O |
| ATOM | 1143 | CB | TYR | A | 148 | 1.909 | 14.622 | 28.300 | 1.00 | 12.19 | | C |
| ANISOU | 1143 | CB | TYR | A | 148 | 1362 | 1931 | 1338 | −419 | −276 | −495 | C |
| ATOM | 1144 | CG | TYR | A | 148 | 0.978 | 13.508 | 28.688 | 1.00 | 14.49 | | C |
| ANISOU | 1144 | CG | TYR | A | 148 | 1774 | 2564 | 1166 | −925 | −38 | −642 | C |
| ATOM | 1145 | CD1 | TYR | A | 148 | 1.407 | 12.336 | 29.268 | 1.00 | 14.74 | | C |
| ANISOU | 1145 | CD1 | TYR | A | 148 | 2197 | 2351 | 1051 | −1051 | 458 | −549 | C |
| ATOM | 1146 | CD2 | TYR | A | 148 | −0.392 | 13.647 | 28.437 | 1.00 | 16.43 | | C |
| ANISOU | 1146 | CD2 | TYR | A | 148 | 1599 | 3350 | 1294 | −974 | 287 | −981 | C |
| ATOM | 1147 | CE1 | TYR | A | 148 | 0.560 | 11.295 | 29.618 | 1.00 | 18.03 | | C |
| ANISOU | 1147 | CE1 | TYR | A | 148 | 2409 | 2419 | 2024 | −1264 | 583 | −843 | C |
| ATOM | 1148 | CE2 | TYR | A | 148 | −1.224 | 12.622 | 28.797 | 1.00 | 18.85 | | C |
| ANISOU | 1148 | CE2 | TYR | A | 148 | 1870 | 2932 | 2361 | −1152 | 338 | −1660 | C |
| ATOM | 1149 | CZ | TYR | A | 148 | −0.780 | 11.476 | 29.387 | 1.00 | 19.06 | | C |
| ANISOU | 1149 | CZ | TYR | A | 148 | 2258 | 3065 | 1920 | −1262 | 831 | −1318 | C |
| ATOM | 1150 | OH | TYR | A | 148 | −1.662 | 10.485 | 29.701 | 1.00 | 22.91 | | O |
| ANISOU | 1150 | OH | TYR | A | 148 | 2439 | 3272 | 2995 | −1365 | 1293 | −1396 | O |
| ATOM | 1151 | N | PRO | A | 149 | 4.829 | 15.877 | 28.271 | 1.00 | 8.57 | | N |
| ANISOU | 1151 | N | PRO | A | 149 | 847 | 1615 | 795 | −64 | −121 | −99 | N |
| ATOM | 1152 | CA | PRO | A | 149 | 5.592 | 16.922 | 27.600 | 1.00 | 8.66 | | C |
| ANISOU | 1152 | CA | PRO | A | 149 | 1065 | 1601 | 624 | 101 | 45 | −123 | C |
| ATOM | 1153 | C | PRO | A | 149 | 4.728 | 17.552 | 26.520 | 1.00 | 8.15 | | C |
| ANISOU | 1153 | C | PRO | A | 149 | 667 | 1345 | 1083 | −177 | −150 | −61 | C |
| ATOM | 1154 | O | PRO | A | 149 | 3.979 | 16.874 | 25.819 | 1.00 | 9.45 | | O |
| ANISOU | 1154 | O | PRO | A | 149 | 1031 | 1332 | 1226 | −50 | −310 | −281 | O |
| ATOM | 1155 | CB | PRO | A | 149 | 6.743 | 16.154 | 26.966 | 1.00 | 10.78 | | C |
| ANISOU | 1155 | CB | PRO | A | 149 | 892 | 2108 | 1097 | 307 | 112 | 90 | C |
| ATOM | 1156 | CG | PRO | A | 149 | 6.839 | 14.854 | 27.606 | 1.00 | 13.72 | | C |
| ANISOU | 1156 | CG | PRO | A | 149 | 1453 | 1728 | 2031 | 248 | 473 | −71 | C |
| ATOM | 1157 | CD | PRO | A | 149 | 5.427 | 14.566 | 28.083 | 1.00 | 10.42 | | C |
| ANISOU | 1157 | CD | PRO | A | 149 | 1201 | 1503 | 1255 | −146 | −142 | −458 | C |
| ATOM | 1158 | N | TYR | A | 150 | 4.858 | 18.845 | 26.355 | 1.00 | 8.10 | | N |
| ANISOU | 1158 | N | TYR | A | 150 | 654 | 1319 | 1104 | −118 | −172 | −105 | N |
| ATOM | 1159 | CA | TYR | A | 150 | 4.125 | 19.673 | 25.415 | 1.00 | 8.29 | | C |
| ANISOU | 1159 | CA | TYR | A | 150 | 1035 | 1314 | 801 | 1 | −198 | −188 | C |
| ATOM | 1160 | C | TYR | A | 150 | 5.101 | 20.459 | 24.544 | 1.00 | 8.16 | | C |
| ANISOU | 1160 | C | TYR | A | 150 | 916 | 1244 | 940 | −86 | −248 | −220 | C |
| ATOM | 1161 | O | TYR | A | 150 | 6.023 | 21.111 | 25.040 | 1.00 | 9.82 | | O |
| ANISOU | 1161 | O | TYR | A | 150 | 1014 | 1343 | 1375 | −97 | −486 | −356 | O |
| ATOM | 1162 | CB | TYR | A | 150 | 3.200 | 20.628 | 26.165 | 1.00 | 8.48 | | C |
| ANISOU | 1162 | CB | TYR | A | 150 | 950 | 1252 | 1021 | −36 | −219 | −269 | C |
| ATOM | 1163 | CG | TYR | A | 150 | 2.485 | 21.597 | 25.224 | 1.00 | 8.94 | | C |
| ANISOU | 1163 | CG | TYR | A | 150 | 646 | 1520 | 1228 | 12 | −220 | −131 | C |
| ATOM | 1164 | CD1 | TYR | A | 150 | 1.682 | 21.126 | 24.198 | 1.00 | 8.54 | | C |
| ANISOU | 1164 | CD1 | TYR | A | 150 | 566 | 1613 | 1066 | −235 | −28 | −44 | C |
| ATOM | 1165 | CD2 | TYR | A | 150 | 2.617 | 22.953 | 25.322 | 1.00 | 10.02 | | C |
| ANISOU | 1165 | CD2 | TYR | A | 150 | 1020 | 1418 | 1370 | 122 | −283 | −217 | C |
| ATOM | 1166 | CE1 | TYR | A | 150 | 1.062 | 22.014 | 23.356 | 1.00 | 9.58 | | C |
| ANISOU | 1166 | CE1 | TYR | A | 150 | 833 | 1852 | 954 | −81 | −232 | −182 | C |
| ATOM | 1167 | CE2 | TYR | A | 150 | 1.993 | 23.866 | 24.489 | 1.00 | 10.25 | | C |
| ANISOU | 1167 | CE2 | TYR | A | 150 | 1041 | 1423 | 1431 | 17 | −176 | −125 | C |
| ATOM | 1168 | CZ | TYR | A | 150 | 1.195 | 23.367 | 23.481 | 1.00 | 11.06 | | C |
| ANISOU | 1168 | CZ | TYR | A | 150 | 1107 | 1773 | 1324 | 313 | −262 | −234 | C |
| ATOM | 1169 | OH | TYR | A | 150 | 0.551 | 24.214 | 22.616 | 1.00 | 13.71 | | O |
| ANISOU | 1169 | OH | TYR | A | 150 | 1356 | 2017 | 1836 | 376 | −493 | −69 | O |
| ATOM | 1170 | N | PHE | A | 151 | 4.858 | 20.391 | 23.221 | 1.00 | 7.68 | | N |
| ANISOU | 1170 | N | PHE | A | 151 | 568 | 1463 | 886 | −41 | −1 | −311 | N |
| ATOM | 1171 | CA | PHE | A | 151 | 5.624 | 21.145 | 22.261 | 1.00 | 8.88 | | C |
| ANISOU | 1171 | CA | PHE | A | 151 | 816 | 1578 | 979 | 8 | −35 | −183 | C |
| ATOM | 1172 | C | PHE | A | 151 | 4.733 | 21.981 | 21.358 | 1.00 | 7.82 | | C |
| ANISOU | 1172 | C | PHE | A | 151 | 655 | 1425 | 891 | −150 | −59 | −301 | C |
| ATOM | 1173 | O | PHE | A | 151 | 3.787 | 21.433 | 20.780 | 1.00 | 9.47 | | O |
| ANISOU | 1173 | O | PHE | A | 151 | 853 | 1302 | 1442 | −143 | −298 | −278 | O |
| ATOM | 1174 | CB | PHE | A | 151 | 6.458 | 20.213 | 21.347 | 1.00 | 9.08 | | C |
| ANISOU | 1174 | CB | PHE | A | 151 | 896 | 1765 | 787 | 62 | 146 | −21 | C |
| ATOM | 1175 | CG | PHE | A | 151 | 7.484 | 19.440 | 22.135 | 1.00 | 8.68 | | C |
| ANISOU | 1175 | CG | PHE | A | 151 | 975 | 1376 | 949 | 16 | −90 | −316 | C |
| ATOM | 1176 | CD1 | PHE | A | 151 | 7.205 | 18.238 | 22.754 | 1.00 | 8.81 | | C |
| ANISOU | 1176 | CD1 | PHE | A | 151 | 841 | 1010 | 1497 | 288 | 24 | −462 | C |
| ATOM | 1177 | CD2 | PHE | A | 151 | 8.759 | 19.941 | 22.312 | 1.00 | 10.73 | | C |
| ANISOU | 1177 | CD2 | PHE | A | 151 | 853 | 1496 | 1728 | 96 | −1 | −131 | C |
| ATOM | 1178 | CE1 | PHE | A | 151 | 8.108 | 17.553 | 23.513 | 1.00 | 10.30 | | C |
| ANISOU | 1178 | CE1 | PHE | A | 151 | 1003 | 928 | 1983 | 252 | −265 | −386 | C |
| ATOM | 1179 | CE2 | PHE | A | 151 | 9.700 | 19.282 | 23.064 | 1.00 | 11.13 | | C |
| ANISOU | 1179 | CE2 | PHE | A | 151 | 910 | 1560 | 1757 | 74 | −152 | −194 | C |
| ATOM | 1180 | CZ | PHE | A | 151 | 9.378 | 18.096 | 23.687 | 1.00 | 11.07 | | C |
| ANISOU | 1180 | CZ | PHE | A | 151 | 1131 | 1490 | 1586 | −8 | −378 | −238 | C |
| ATOM | 1181 | N | GLU | A | 152 | 5.071 | 23.237 | 21.204 | 1.00 | 8.91 | | N |
| ANISOU | 1181 | N | GLU | A | 152 | 897 | 1407 | 1083 | −215 | −146 | −210 | N |
| ATOM | 1182 | CA | GLU | A | 152 | 4.504 | 24.114 | 20.192 | 1.00 | 9.73 | | C |
| ANISOU | 1182 | CA | GLU | A | 152 | 812 | 1651 | 1235 | −151 | −243 | −104 | C |
| ATOM | 1183 | C | GLU | A | 152 | 5.313 | 23.985 | 18.933 | 1.00 | 9.29 | | C |
| ANISOU | 1183 | C | GLU | A | 152 | 813 | 1491 | 1227 | 64 | −276 | −2 | C |
| ATOM | 1184 | O | GLU | A | 152 | 6.491 | 24.287 | 18.931 | 1.00 | 12.03 | | O |
| ANISOU | 1184 | O | GLU | A | 152 | 856 | 2093 | 1623 | −277 | −132 | −275 | O |
| ATOM | 1185 | CB | GLU | A | 152 | 4.482 | 25.529 | 20.781 | 1.00 | 12.37 | | C |
| ANISOU | 1185 | CB | GLU | A | 152 | 2002 | 1469 | 1230 | −56 | 184 | 98 | C |
| ATOM | 1186 | CG | GLU | A | 152 | 3.405 | 25.551 | 21.876 | 1.00 | 17.61 | | C |
| ANISOU | 1186 | CG | GLU | A | 152 | 2124 | 2638 | 1930 | 690 | 532 | −96 | C |
| ATOM | 1187 | CD | GLU | A | 152 | 2.574 | 26.799 | 21.948 | 1.00 | 14.84 | | C |
| ANISOU | 1187 | CD | GLU | A | 152 | 1507 | 2267 | 1865 | 273 | −261 | −231 | C |
| ATOM | 1188 | OE1 | GLU | A | 152 | 2.978 | 27.843 | 21.442 | 1.00 | 18.75 | | O |
| ANISOU | 1188 | OE1 | GLU | A | 152 | 1846 | 2282 | 2998 | 1 | 264 | −332 | O |
| ATOM | 1189 | OE2 | GLU | A | 152 | 1.476 | 26.692 | 22.542 | 1.00 | 15.91 | | O |
| ANISOU | 1189 | OE2 | GLU | A | 152 | 1681 | 2126 | 2239 | 545 | −30 | 167 | O |
| ATOM | 1190 | N | THR | A | 153 | 4.724 | 23.485 | 17.873 | 1.00 | 8.87 | | N |
| ANISOU | 1190 | N | THR | A | 153 | 779 | 1400 | 1191 | 51 | −151 | −70 | N |
| ATOM | 1191 | CA | THR | A | 153 | 5.422 | 23.085 | 16.683 | 1.00 | 9.19 | | C |
| ANISOU | 1191 | CA | THR | A | 153 | 656 | 1577 | 1258 | 50 | −10 | −25 | C |
| ATOM | 1192 | C | THR | A | 153 | 4.944 | 23.832 | 15.458 | 1.00 | 9.15 | | C |
| ANISOU | 1192 | C | THR | A | 153 | 726 | 1624 | 1126 | −21 | −154 | −148 | C |
| ATOM | 1193 | O | THR | A | 153 | 3.823 | 24.328 | 15.396 | 1.00 | 10.53 | | O |
| ANISOU | 1193 | O | THR | A | 153 | 854 | 1682 | 1466 | 106 | −196 | −56 | O |
| ATOM | 1194 | CB | THR | A | 153 | 5.314 | 21.564 | 16.368 | 1.00 | 9.45 | | C |
| ANISOU | 1194 | CB | THR | A | 153 | 659 | 1633 | 1298 | 349 | −206 | −179 | C |
| ATOM | 1195 | OG1 | THR | A | 153 | 3.960 | 21.177 | 16.113 | 1.00 | 9.89 | | O |
| ANISOU | 1195 | OG1 | THR | A | 153 | 819 | 1603 | 1337 | 43 | −247 | −53 | O |
| ATOM | 1196 | CG2 | THR | A | 153 | 5.799 | 20.688 | 17.500 | 1.00 | 11.84 | | C |
| ANISOU | 1196 | CG2 | THR | A | 153 | 1114 | 1795 | 1592 | 124 | −388 | 202 | C |
| ATOM | 1197 | N | SER | A | 154 | 5.789 | 23.841 | 14.440 | 1.00 | 10.66 | | N |
| ANISOU | 1197 | N | SER | A | 154 | 1022 | 1981 | 1048 | 54 | −92 | −261 | N |
| ATOM | 1198 | CA | SER | A | 154 | 5.376 | 24.225 | 13.096 | 1.00 | 9.68 | | C |
| ANISOU | 1198 | CA | SER | A | 154 | 643 | 1848 | 1187 | −120 | −96 | −53 | C |
| ATOM | 1199 | C | SER | A | 154 | 6.018 | 23.315 | 12.063 | 1.00 | 9.24 | | C |
| ANISOU | 1199 | C | SER | A | 154 | 984 | 1650 | 877 | −69 | −43 | 204 | C |
| ATOM | 1200 | O | SER | A | 154 | 7.232 | 23.359 | 11.872 | 1.00 | 10.95 | | O |
| ANISOU | 1200 | O | SER | A | 154 | 910 | 1993 | 1258 | 106 | −77 | −91 | O |
| ATOM | 1201 | CB | SER | A | 154 | 5.748 | 25.677 | 12.857 | 1.00 | 10.16 | | C |
| ANISOU | 1201 | CB | SER | A | 154 | 757 | 1867 | 1238 | −239 | 34 | −174 | C |
| ATOM | 1202 | OG | SER | A | 154 | 5.305 | 26.055 | 11.540 | 1.00 | 13.60 | | O |
| ANISOU | 1202 | OG | SER | A | 154 | 1772 | 1993 | 1403 | −355 | −173 | 77 | O |
| ATOM | 1203 | N | ALA | A | 155 | 5.254 | 22.483 | 11.368 | 1.00 | 10.35 | | N |
| ANISOU | 1203 | N | ALA | A | 155 | 1059 | 1662 | 1210 | −72 | −167 | 71 | N |
| ATOM | 1204 | CA | ALA | A | 155 | 5.810 | 21.733 | 10.254 | 1.00 | 9.76 | | C |
| ANISOU | 1204 | CA | ALA | A | 155 | 961 | 1600 | 1147 | 78 | −417 | 71 | C |
| ATOM | 1205 | C | ALA | A | 155 | 6.274 | 22.678 | 9.183 | 1.00 | 9.21 | | C |
| ANISOU | 1205 | C | ALA | A | 155 | 906 | 1392 | 1202 | 101 | −273 | −130 | C |
| ATOM | 1206 | O | ALA | A | 155 | 7.201 | 22.376 | 8.452 | 1.00 | 11.90 | | O |
| ANISOU | 1206 | O | ALA | A | 155 | 1008 | 1910 | 1604 | 398 | −11 | 184 | O |
| ATOM | 1207 | CB | ALA | A | 155 | 4.806 | 20.742 | 9.696 | 1.00 | 11.78 | | C |
| ANISOU | 1207 | CB | ALA | A | 155 | 1084 | 1782 | 1609 | −179 | −77 | −180 | C |
| ATOM | 1208 | N | LYS | A | 156 | 5.603 | 23.837 | 9.057 | 1.00 | 9.71 | | N |
| ANISOU | 1208 | N | LYS | A | 156 | 987 | 1295 | 1407 | 63 | −257 | −56 | N |
| ATOM | 1209 | CA | LYS | A | 156 | 5.898 | 24.743 | 7.962 | 1.00 | 10.88 | | C |
| ANISOU | 1209 | CA | LYS | A | 156 | 1255 | 1569 | 1312 | 30 | −313 | 80 | C |
| ATOM | 1210 | C | LYS | A | 156 | 7.256 | 25.376 | 8.093 | 1.00 | 11.55 | | C |
| ANISOU | 1210 | C | LYS | A | 156 | 1221 | 1745 | 1422 | −5 | −322 | 471 | C |
| ATOM | 1211 | O | LYS | A | 156 | 8.026 | 25.553 | 7.154 | 1.00 | 15.52 | | O |
| ANISOU | 1211 | O | LYS | A | 156 | 1477 | 2660 | 1762 | −144 | 83 | 131 | O |
| ATOM | 1212 | CB | LYS | A | 156 | 4.743 | 25.764 | 7.908 | 1.00 | 11.18 | | C |
| ANISOU | 1212 | CB | LYS | A | 156 | 1279 | 1512 | 1458 | 27 | −19 | 330 | C |
| ATOM | 1213 | CG | LYS | A | 156 | 4.884 | 26.730 | 6.722 | 1.00 | 10.85 | | C |
| ANISOU | 1213 | CG | LYS | A | 156 | 1436 | 1540 | 1147 | 267 | 166 | 177 | C |
| ATOM | 1214 | CD | LYS | A | 156 | 3.624 | 27.586 | 6.613 | 1.00 | 11.99 | | C |
| ANISOU | 1214 | CD | LYS | A | 156 | 1319 | 1574 | 1663 | 157 | −164 | 245 | C |
| ATOM | 1215 | CE | LYS | A | 156 | 3.691 | 28.470 | 5.373 | 1.00 | 13.34 | | C |
| ANISOU | 1215 | CE | LYS | A | 156 | 1359 | 1811 | 1898 | −5 | −354 | 495 | C |
| ATOM | 1216 | NZ | LYS | A | 156 | 2.394 | 29.220 | 5.300 | 1.00 | 14.90 | | N |
| ANISOU | 1216 | NZ | LYS | A | 156 | 2151 | 1753 | 1756 | 643 | 41 | 329 | N |
| ATOM | 1217 | N | ASP | A | 157 | 7.760 | 25.720 | 9.243 | 1.00 | 14.01 | | N |
| ANISOU | 1217 | N | ASP | A | 157 | 1612 | 2170 | 1543 | −703 | −356 | 505 | N |
| ATOM | 1218 | CA | ASP | A | 157 | 8.986 | 26.400 | 9.559 | 1.00 | 14.79 | | C |
| ANISOU | 1218 | CA | ASP | A | 157 | 1560 | 2025 | 2034 | −780 | −341 | 585 | C |
| ATOM | 1219 | C | ASP | A | 157 | 9.941 | 25.531 | 10.368 | 1.00 | 15.21 | | C |
| ANISOU | 1219 | C | ASP | A | 157 | 1610 | 2428 | 1742 | −689 | −391 | 612 | C |
| ATOM | 1220 | O | ASP | A | 157 | 11.043 | 26.044 | 10.630 | 1.00 | 16.05 | | O |
| ANISOU | 1220 | O | ASP | A | 157 | 1577 | 2189 | 2332 | −596 | −370 | 346 | O |
| ATOM | 1221 | CB | ASP | A | 157 | 8.714 | 27.744 | 10.214 | 1.00 | 20.11 | | C |
| ANISOU | 1221 | CB | ASP | A | 157 | 1818 | 2464 | 3358 | −530 | −744 | −205 | C |
| ATOM | 1222 | CG | ASP | A | 157 | 8.372 | 27.893 | 11.644 | 1.00 | 23.56 | | C |
| ANISOU | 1222 | CG | ASP | A | 157 | 3578 | 2307 | 3068 | −37 | −1247 | −448 | C |
| ATOM | 1223 | OD1 | ASP | A | 157 | 8.567 | 26.913 | 12.391 | 1.00 | 23.44 | | O |
| ANISOU | 1223 | OD1 | ASP | A | 157 | 2417 | 2708 | 3781 | −541 | −644 | 316 | O |
| ATOM | 1224 | OD2 | ASP | A | 157 | 7.949 | 29.039 | 11.954 | 1.00 | 28.47 | | O |
| ANISOU | 1224 | OD2 | ASP | A | 157 | 4485 | 1986 | 4346 | −700 | 352 | −461 | O |
| ATOM | 1225 | N | ALA | A | 158 | 9.606 | 24.300 | 10.713 | 1.00 | 12.40 | | N |
| ANISOU | 1225 | N | ALA | A | 158 | 1121 | 1916 | 1676 | −299 | −324 | −18 | N |
| ATOM | 1226 | CA | ALA | A | 158 | 10.359 | 23.288 | 11.407 | 1.00 | 12.12 | | C |
| ANISOU | 1226 | CA | ALA | A | 158 | 1087 | 1850 | 1670 | −135 | −315 | −224 | C |
| ATOM | 1227 | C | ALA | A | 158 | 10.431 | 23.487 | 12.920 | 1.00 | 11.89 | | C |
| ANISOU | 1227 | C | ALA | A | 158 | 882 | 1969 | 1668 | 184 | −254 | −191 | C |
| ATOM | 1228 | O | ALA | A | 158 | 10.977 | 22.617 | 13.612 | 1.00 | 13.18 | | O |
| ANISOU | 1228 | O | ALA | A | 158 | 1496 | 1744 | 1768 | 93 | −139 | 17 | O |
| ATOM | 1229 | CB | ALA | A | 158 | 11.783 | 23.158 | 10.900 | 1.00 | 16.06 | | C |
| ANISOU | 1229 | CB | ALA | A | 158 | 1249 | 3194 | 1660 | 178 | −189 | −383 | C |
| ATOM | 1230 | N | THR | A | 159 | 9.864 | 24.539 | 13.468 | 1.00 | 11.36 | | N |
| ANISOU | 1230 | N | THR | A | 159 | 959 | 1841 | 1517 | 91 | −261 | −62 | N |
| ATOM | 1231 | CA | THR | A | 159 | 9.974 | 24.796 | 14.911 | 1.00 | 11.43 | | C |
| ANISOU | 1231 | CA | THR | A | 159 | 1067 | 1705 | 1569 | −82 | −162 | −131 | C |
| ATOM | 1232 | C | THR | A | 159 | 9.557 | 23.615 | 15.776 | 1.00 | 10.55 | | C |
| ANISOU | 1232 | C | THR | A | 159 | 820 | 1629 | 1561 | 57 | −238 | −131 | C |
| ATOM | 1233 | O | THR | A | 159 | 8.431 | 23.138 | 15.675 | 1.00 | 10.81 | | O |
| ANISOU | 1233 | O | THR | A | 159 | 766 | 1814 | 1527 | 23 | −277 | −101 | O |
| ATOM | 1234 | CB | THR | A | 159 | 9.097 | 26.004 | 15.283 | 1.00 | 13.75 | | C |
| ANISOU | 1234 | CB | THR | A | 159 | 1669 | 1427 | 2131 | −108 | 0 | −295 | C |
| ATOM | 1235 | OG1 | THR | A | 159 | 9.455 | 27.168 | 14.528 | 1.00 | 17.97 | | O |
| ANISOU | 1235 | OG1 | THR | A | 159 | 2294 | 1734 | 2799 | −23 | −106 | 306 | O |
| ATOM | 1236 | CG2 | THR | A | 159 | 9.275 | 26.407 | 16.735 | 1.00 | 14.25 | | C |
| ANISOU | 1236 | CG2 | THR | A | 159 | 1220 | 1966 | 2229 | 27 | 26 | −521 | C |
| ATOM | 1237 | N | ASN | A | 160 | 10.452 | 23.145 | 16.621 | 1.00 | 10.38 | | N |
| ANISOU | 1237 | N | ASN | A | 160 | 780 | 1688 | 1476 | 101 | −273 | −256 | N |
| ATOM | 1238 | CA | ASN | A | 160 | 10.297 | 22.129 | 17.611 | 1.00 | 11.15 | | C |
| ANISOU | 1238 | CA | ASN | A | 160 | 918 | 1666 | 1654 | 240 | −153 | −177 | C |
| ATOM | 1239 | C | ASN | A | 160 | 9.903 | 20.769 | 17.067 | 1.00 | 11.35 | | C |
| ANISOU | 1239 | C | ASN | A | 160 | 946 | 1862 | 1506 | −166 | −315 | −186 | C |
| ATOM | 1240 | O | ASN | A | 160 | 9.534 | 19.882 | 17.863 | 1.00 | 12.10 | | O |
| ANISOU | 1240 | O | ASN | A | 160 | 1111 | 1857 | 1632 | 34 | 129 | −191 | O |
| ATOM | 1241 | CB | ASN | A | 160 | 9.232 | 22.547 | 18.657 | 1.00 | 12.48 | | C |
| ANISOU | 1241 | CB | ASN | A | 160 | 1160 | 2227 | 1355 | 242 | −168 | −180 | C |
| ATOM | 1242 | CG | ASN | A | 160 | 9.799 | 23.453 | 19.698 | 1.00 | 12.57 | | C |
| ANISOU | 1242 | CG | ASN | A | 160 | 1217 | 2394 | 1164 | 597 | −556 | −97 | C |
| ATOM | 1243 | OD1 | ASN | A | 160 | 10.971 | 23.465 | 19.984 | 1.00 | 18.28 | | O |
| ANISOU | 1243 | OD1 | ASN | A | 160 | 1290 | 3801 | 1854 | 621 | −807 | −705 | O |
| ATOM | 1244 | ND2 | ASN | A | 160 | 8.880 | 24.272 | 20.245 | 1.00 | 18.05 | | N |
| ANISOU | 1244 | ND2 | ASN | A | 160 | 1612 | 2942 | 2305 | 691 | −320 | −850 | N |
| ATOM | 1245 | N | VAL | A | 161 | 10.020 | 20.541 | 15.766 | 1.00 | 11.35 | | N |
| ANISOU | 1245 | N | VAL | A | 161 | 1010 | 1809 | 1493 | −136 | −404 | −95 | N |
| ATOM | 1246 | CA | VAL | A | 161 | 9.540 | 19.271 | 15.214 | 1.00 | 10.11 | | C |
| ANISOU | 1246 | CA | VAL | A | 161 | 889 | 1637 | 1314 | 96 | 155 | −240 | C |
| ATOM | 1247 | C | VAL | A | 161 | 10.469 | 18.153 | 15.637 | 1.00 | 10.52 | | C |
| ANISOU | 1247 | C | VAL | A | 161 | 994 | 1889 | 1114 | 126 | −59 | −177 | C |
| ATOM | 1248 | O | VAL | A | 161 | 10.014 | 17.155 | 16.193 | 1.00 | 10.91 | | O |
| ANISOU | 1248 | O | VAL | A | 161 | 1063 | 1803 | 1280 | 110 | −228 | −124 | O |
| ATOM | 1249 | CB | VAL | A | 161 | 9.373 | 19.349 | 13.698 | 1.00 | 10.97 | | C |
| ANISOU | 1249 | CB | VAL | A | 161 | 1347 | 1428 | 1392 | 74 | −186 | −177 | C |
| ATOM | 1250 | CG1 | VAL | A | 161 | 9.007 | 17.997 | 13.091 | 1.00 | 12.92 | | C |
| ANISOU | 1250 | CG1 | VAL | A | 161 | 1949 | 1417 | 1544 | 68 | −455 | −213 | C |
| ATOM | 1251 | CG2 | VAL | A | 161 | 8.275 | 20.381 | 13.378 | 1.00 | 11.63 | | C |
| ANISOU | 1251 | CG2 | VAL | A | 161 | 1221 | 1426 | 1771 | −26 | −265 | −253 | C |
| ATOM | 1252 | N | ALA | A | 162 | 11.780 | 18.291 | 15.391 | 1.00 | 10.17 | | N |
| ANISOU | 1252 | N | ALA | A | 162 | 935 | 1789 | 1141 | 197 | −171 | −457 | N |
| ATOM | 1253 | CA | ALA | A | 162 | 12.657 | 17.221 | 15.793 | 1.00 | 10.44 | | C |
| ANISOU | 1253 | CA | ALA | A | 162 | 1067 | 1510 | 1389 | 106 | 21 | −298 | C |
| ATOM | 1254 | C | ALA | A | 162 | 12.641 | 17.005 | 17.285 | 1.00 | 10.52 | | C |
| ANISOU | 1254 | C | ALA | A | 162 | 1044 | 1560 | 1395 | −27 | −150 | −220 | C |
| ATOM | 1255 | O | ALA | A | 162 | 12.672 | 15.871 | 17.751 | 1.00 | 11.38 | | O |
| ANISOU | 1255 | O | ALA | A | 162 | 1148 | 1610 | 1566 | 87 | −149 | −116 | O |
| ATOM | 1256 | CB | ALA | A | 162 | 14.074 | 17.529 | 15.285 | 1.00 | 11.67 | | C |
| ANISOU | 1256 | CB | ALA | A | 162 | 905 | 2121 | 1410 | 209 | −18 | −452 | C |
| ATOM | 1257 | N | ALA | A | 163 | 12.567 | 18.076 | 18.054 | 1.00 | 10.29 | | N |
| ANISOU | 1257 | N | ALA | A | 163 | 1107 | 1561 | 1243 | −31 | −377 | −106 | N |
| ATOM | 1258 | CA | ALA | A | 163 | 12.565 | 17.967 | 19.503 | 1.00 | 9.97 | | C |
| ANISOU | 1258 | CA | ALA | A | 163 | 931 | 1581 | 1277 | 111 | −199 | −170 | C |
| ATOM | 1259 | C | ALA | A | 163 | 11.401 | 17.132 | 20.006 | 1.00 | 10.33 | | C |
| ANISOU | 1259 | C | ALA | A | 163 | 934 | 1526 | 1466 | 213 | −108 | −100 | C |
| ATOM | 1260 | O | ALA | A | 163 | 11.517 | 16.372 | 20.980 | 1.00 | 11.44 | | O |
| ANISOU | 1260 | O | ALA | A | 163 | 1300 | 1452 | 1593 | 149 | −159 | −28 | O |
| ATOM | 1261 | CB | ALA | A | 163 | 12.587 | 19.345 | 20.156 | 1.00 | 13.07 | | C |
| ANISOU | 1261 | CB | ALA | A | 163 | 2147 | 1508 | 1311 | −45 | 152 | −173 | C |
| ATOM | 1262 | N | ALA | A | 164 | 10.242 | 17.293 | 19.361 | 1.00 | 10.98 | | N |
| ANISOU | 1262 | N | ALA | A | 164 | 896 | 2001 | 1276 | 50 | −96 | −250 | N |
| ATOM | 1263 | CA | ALA | A | 164 | 9.083 | 16.581 | 19.827 | 1.00 | 9.96 | | C |
| ANISOU | 1263 | CA | ALA | A | 164 | 930 | 1648 | 1206 | 155 | −106 | −223 | C |
| ATOM | 1264 | C | ALA | A | 164 | 9.243 | 15.088 | 19.603 | 1.00 | 10.16 | | C |
| ANISOU | 1264 | C | ALA | A | 164 | 832 | 1721 | 1308 | 369 | −232 | −267 | C |
| ATOM | 1265 | O | ALA | A | 164 | 8.909 | 14.251 | 20.457 | 1.00 | 10.71 | | O |
| ANISOU | 1265 | O | ALA | A | 164 | 1219 | 1523 | 1327 | 297 | −89 | −370 | O |
| ATOM | 1266 | CB | ALA | A | 164 | 7.838 | 17.091 | 19.102 | 1.00 | 10.61 | | C |
| ANISOU | 1266 | CB | ALA | A | 164 | 830 | 1734 | 1466 | 268 | 108 | 124 | C |
| ATOM | 1267 | N | PHE | A | 165 | 9.721 | 14.694 | 18.440 | 1.00 | 10.01 | | N |
| ANISOU | 1267 | N | PHE | A | 165 | 838 | 1571 | 1394 | −94 | −69 | −323 | N |
| ATOM | 1268 | CA | PHE | A | 165 | 9.921 | 13.290 | 18.142 | 1.00 | 10.26 | | C |
| ANISOU | 1268 | CA | PHE | A | 165 | 827 | 1666 | 1404 | −77 | 29 | −425 | C |
| ATOM | 1269 | C | PHE | A | 165 | 11.065 | 12.670 | 18.949 | 1.00 | 10.90 | | C |
| ANISOU | 1269 | C | PHE | A | 165 | 1457 | 1360 | 1325 | −192 | −407 | −266 | C |
| ATOM | 1270 | O | PHE | A | 165 | 11.041 | 11.539 | 19.381 | 1.00 | 11.69 | | O |
| ANISOU | 1270 | O | PHE | A | 165 | 1201 | 1358 | 1883 | −151 | −3 | −294 | O |
| ATOM | 1271 | CB | PHE | A | 165 | 10.184 | 13.101 | 16.637 | 1.00 | 10.57 | | C |
| ANISOU | 1271 | CB | PHE | A | 165 | 1251 | 1554 | 1213 | 40 | −186 | −317 | C |
| ATOM | 1272 | CG | PHE | A | 165 | 8.940 | 13.203 | 15.791 | 1.00 | 10.17 | | C |
| ANISOU | 1272 | CG | PHE | A | 165 | 975 | 1366 | 1524 | 69 | −85 | −350 | C |
| ATOM | 1273 | CD1 | PHE | A | 165 | 8.054 | 12.137 | 15.736 | 1.00 | 14.16 | | C |
| ANISOU | 1273 | CD1 | PHE | A | 165 | 1465 | 1644 | 2269 | −279 | −308 | −492 | C |
| ATOM | 1274 | CD2 | PHE | A | 165 | 8.677 | 14.333 | 15.059 | 1.00 | 13.26 | | C |
| ANISOU | 1274 | CD2 | PHE | A | 165 | 1641 | 1785 | 1612 | −64 | −487 | −54 | C |
| ATOM | 1275 | CE1 | PHE | A | 165 | 6.922 | 12.260 | 14.958 | 1.00 | 13.09 | | C |
| ANISOU | 1275 | CE1 | PHE | A | 165 | 1824 | 1377 | 1772 | −619 | −409 | −238 | C |
| ATOM | 1276 | CE2 | PHE | A | 165 | 7.543 | 14.457 | 14.268 | 1.00 | 12.36 | | C |
| ANISOU | 1276 | CE2 | PHE | A | 165 | 1495 | 1325 | 1877 | −262 | −474 | 117 | C |
| ATOM | 1277 | CZ | PHE | A | 165 | 6.659 | 13.387 | 14.227 | 1.00 | 11.98 | | C |
| ANISOU | 1277 | CZ | PHE | A | 165 | 1726 | 1429 | 1398 | −484 | 120 | −234 | C |
| ATOM | 1278 | N | GLU | A | 166 | 12.127 | 13.453 | 19.210 | 1.00 | 10.36 | | N |
| ANISOU | 1278 | N | GLU | A | 166 | 899 | 1310 | 1727 | 54 | −110 | −119 | N |
| ATOM | 1279 | CA | GLU | A | 166 | 13.232 | 13.001 | 20.048 | 1.00 | 10.19 | | C |
| ANISOU | 1279 | CA | GLU | A | 166 | 854 | 1397 | 1621 | 186 | −32 | −228 | C |
| ATOM | 1280 | C | GLU | A | 166 | 12.749 | 12.757 | 21.462 | 1.00 | 10.09 | | C |
| ANISOU | 1280 | C | GLU | A | 166 | 875 | 1310 | 1648 | 190 | −37 | −203 | C |
| ATOM | 1281 | O | GLU | A | 166 | 13.123 | 11.810 | 22.146 | 1.00 | 11.26 | | O |
| ANISOU | 1281 | O | GLU | A | 166 | 1014 | 1504 | 1759 | 187 | −106 | −30 | O |
| ATOM | 1282 | CB | GLU | A | 166 | 14.382 | 14.023 | 19.974 | 1.00 | 11.89 | | C |
| ANISOU | 1282 | CB | GLU | A | 166 | 1088 | 1627 | 1802 | −125 | −170 | −64 | C |
| ATOM | 1283 | CG | GLU | A | 166 | 15.085 | 14.030 | 18.623 | 1.00 | 13.19 | | C |
| ANISOU | 1283 | CG | GLU | A | 166 | 1008 | 1766 | 2239 | 370 | 260 | 65 | C |
| ATOM | 1284 | CD | GLU | A | 166 | 15.788 | 15.283 | 18.175 | 1.00 | 17.63 | | C |
| ANISOU | 1284 | CD | GLU | A | 166 | 2032 | 2201 | 2465 | −170 | 638 | 87 | C |
| ATOM | 1285 | OE1 | GLU | A | 166 | 15.656 | 16.406 | 18.726 | 1.00 | 23.73 | | O |
| ANISOU | 1285 | OE1 | GLU | A | 166 | 2380 | 2061 | 4574 | −117 | 248 | −345 | O |
| ATOM | 1286 | OE2 | GLU | A | 166 | 16.541 | 15.155 | 17.176 | 1.00 | 27.22 | | O |
| ANISOU | 1286 | OE2 | GLU | A | 166 | 3000 | 4457 | 2884 | −738 | 1430 | 98 | O |
| ATOM | 1287 | N | GLU | A | 167 | 11.840 | 13.617 | 21.941 | 1.00 | 10.18 | | N |
| ANISOU | 1287 | N | GLU | A | 167 | 962 | 1359 | 1545 | 198 | −50 | −200 | N |
| ATOM | 1288 | CA | GLU | A | 167 | 11.296 | 13.424 | 23.269 | 1.00 | 9.91 | | C |
| ANISOU | 1288 | CA | GLU | A | 167 | 705 | 1549 | 1513 | 139 | −155 | −186 | C |
| ATOM | 1289 | C | GLU | A | 167 | 10.451 | 12.162 | 23.300 | 1.00 | 10.80 | | C |
| ANISOU | 1289 | C | GLU | A | 167 | 878 | 1770 | 1457 | −41 | 45 | −364 | C |
| ATOM | 1290 | O | GLU | A | 167 | 10.509 | 11.492 | 24.334 | 1.00 | 11.29 | | O |
| ANISOU | 1290 | O | GLU | A | 167 | 1144 | 1656 | 1490 | −50 | 50 | −302 | O |
| ATOM | 1291 | CB | GLU | A | 167 | 10.504 | 14.637 | 23.734 | 1.00 | 11.26 | | C |
| ANISOU | 1291 | CB | GLU | A | 167 | 996 | 1757 | 1525 | 439 | −72 | −157 | C |
| ATOM | 1292 | CG | GLU | A | 167 | 9.817 | 14.533 | 25.074 | 1.00 | 9.94 | | C |
| ANISOU | 1292 | CG | GLU | A | 167 | 1312 | 1161 | 1305 | 16 | −168 | −243 | C |
| ATOM | 1293 | CD | GLU | A | 167 | 10.805 | 14.408 | 26.254 | 1.00 | 14.27 | | C |
| ANISOU | 1293 | CD | GLU | A | 167 | 1814 | 2191 | 1418 | 79 | −476 | −942 | C |
| ATOM | 1294 | OE1 | GLU | A | 167 | 11.996 | 14.672 | 26.072 | 1.00 | 23.46 | | O |
| ANISOU | 1294 | OE1 | GLU | A | 167 | 1455 | 5212 | 2247 | 272 | −529 | −1590 | O |
| ATOM | 1295 | OE2 | GLU | A | 167 | 10.342 | 14.096 | 27.348 | 1.00 | 24.77 | | O |
| ANISOU | 1295 | OE2 | GLU | A | 167 | 3484 | 4571 | 1355 | −1181 | −784 | −216 | O |
| ATOM | 1296 | N | ALA | A | 168 | 9.751 | 11.829 | 22.231 | 1.00 | 10.34 | | N |
| ANISOU | 1296 | N | ALA | A | 168 | 1012 | 1408 | 1507 | 54 | 4 | −338 | N |
| ATOM | 1297 | CA | ALA | A | 168 | 8.972 | 10.587 | 22.261 | 1.00 | 10.66 | | C |
| ANISOU | 1297 | CA | ALA | A | 168 | 1072 | 1560 | 1417 | −48 | −127 | −192 | C |
| ATOM | 1298 | C | ALA | A | 168 | 9.861 | 9.394 | 22.583 | 1.00 | 10.96 | | C |
| ANISOU | 1298 | C | ALA | A | 168 | 1048 | 1462 | 1652 | −163 | −6 | −91 | C |
| ATOM | 1299 | O | ALA | A | 168 | 9.530 | 8.545 | 23.411 | 1.00 | 11.76 | | O |
| ANISOU | 1299 | O | ALA | A | 168 | 1435 | 1554 | 1481 | −204 | −68 | −92 | O |
| ATOM | 1300 | CB | ALA | A | 168 | 8.259 | 10.385 | 20.947 | 1.00 | 13.66 | | C |
| ANISOU | 1300 | CB | ALA | A | 168 | 1287 | 2278 | 1626 | −451 | −370 | −66 | C |
| ATOM | 1301 | N | VAL | A | 169 | 11.002 | 9.352 | 21.875 | 1.00 | 10.87 | | N |
| ANISOU | 1301 | N | VAL | A | 169 | 1150 | 1476 | 1505 | −42 | −5 | −331 | N |
| ATOM | 1302 | CA | VAL | A | 169 | 11.887 | 8.214 | 22.133 | 1.00 | 12.67 | | C |
| ANISOU | 1302 | CA | VAL | A | 169 | 1272 | 1516 | 2026 | −13 | −148 | −297 | C |
| ATOM | 1303 | C | VAL | A | 169 | 12.443 | 8.250 | 23.551 | 1.00 | 13.40 | | C |
| ANISOU | 1303 | C | VAL | A | 169 | 1703 | 1231 | 2158 | 180 | −434 | −355 | C |
| ATOM | 1304 | O | VAL | A | 169 | 12.536 | 7.225 | 24.219 | 1.00 | 13.96 | | O |
| ANISOU | 1304 | O | VAL | A | 169 | 1609 | 1216 | 2480 | 73 | −731 | −281 | O |
| ATOM | 1305 | CB | VAL | A | 169 | 12.999 | 8.168 | 21.098 | 1.00 | 15.57 | | C |
| ANISOU | 1305 | CB | VAL | A | 169 | 1397 | 2046 | 2474 | 305 | 112 | −596 | C |
| ATOM | 1306 | CG1 | VAL | A | 169 | 14.081 | 7.192 | 21.518 | 1.00 | 18.56 | | C |
| ANISOU | 1306 | CG1 | VAL | A | 169 | 1994 | 2251 | 2807 | 741 | −27 | −614 | C |
| ATOM | 1307 | CG2 | VAL | A | 169 | 12.401 | 7.774 | 19.760 | 1.00 | 18.33 | | C |
| ANISOU | 1307 | CG2 | VAL | A | 169 | 2217 | 2628 | 2120 | 614 | 156 | −460 | C |
| ATOM | 1308 | N | ARG | A | 170 | 12.818 | 9.427 | 24.073 | 1.00 | 11.80 | | N |
| ANISOU | 1308 | N | ARG | A | 170 | 1154 | 1374 | 1957 | −181 | −241 | −10 | N |
| ATOM | 1309 | CA | ARG | A | 170 | 13.250 | 9.514 | 25.441 | 1.00 | 12.31 | | C |
| ANISOU | 1309 | CA | ARG | A | 170 | 1111 | 1497 | 2071 | 144 | −287 | −439 | C |
| ATOM | 1310 | C | ARG | A | 170 | 12.193 | 9.020 | 26.421 | 1.00 | 13.02 | | C |
| ANISOU | 1310 | C | ARG | A | 170 | 1194 | 2071 | 1681 | 60 | −502 | −210 | C |
| ATOM | 1311 | O | ARG | A | 170 | 12.476 | 8.306 | 27.387 | 1.00 | 15.66 | | O |
| ANISOU | 1311 | O | ARG | A | 170 | 1540 | 2223 | 2186 | −305 | −887 | 145 | O |
| ATOM | 1312 | CB | ARG | A | 170 | 13.659 | 10.939 | 25.809 | 1.00 | 12.92 | | C |
| ANISOU | 1312 | CB | ARG | A | 170 | 1357 | 1312 | 2239 | 447 | −308 | −407 | C |
| ATOM | 1313 | CG | ARG | A | 170 | 14.095 | 11.127 | 27.246 | 1.00 | 15.07 | | C |
| ANISOU | 1313 | CG | ARG | A | 170 | 1723 | 1538 | 2463 | 108 | −630 | −582 | C |
| ATOM | 1314 | CD | ARG | A | 170 | 15.239 | 10.167 | 27.617 | 1.00 | 16.06 | | C |
| ANISOU | 1314 | CD | ARG | A | 170 | 1651 | 1539 | 2913 | −222 | −864 | 168 | C |
| ATOM | 1315 | NE | ARG | A | 170 | 16.467 | 10.533 | 26.999 | 1.00 | 17.37 | | N |
| ANISOU | 1315 | NE | ARG | A | 170 | 1671 | 1408 | 3521 | −128 | −464 | −375 | N |
| ATOM | 1316 | CZ | ARG | A | 170 | 17.624 | 9.896 | 27.078 | 1.00 | 18.71 | | C |
| ANISOU | 1316 | CZ | ARG | A | 170 | 2100 | 1879 | 3131 | 422 | −269 | −529 | C |
| ATOM | 1317 | NH1 | ARG | A | 170 | 17.701 | 8.784 | 27.754 | 1.00 | 19.82 | | N |
| ANISOU | 1317 | NH1 | ARG | A | 170 | 2478 | 2336 | 2716 | 731 | −398 | −314 | N |
| ATOM | 1318 | NH2 | ARG | A | 170 | 18.713 | 10.375 | 26.480 | 1.00 | 22.40 | | N |
| ANISOU | 1318 | NH2 | ARG | A | 170 | 1822 | 2140 | 4551 | 520 | −150 | −175 | N |
| ATOM | 1319 | N | ARG | A | 171 | 10.921 | 9.344 | 26.209 | 1.00 | 12.37 | | N |
| ANISOU | 1319 | N | ARG | A | 171 | 1145 | 1324 | 2233 | −58 | −335 | −14 | N |
| ATOM | 1320 | CA | ARG | A | 171 | 9.862 | 8.913 | 27.101 | 1.00 | 12.44 | | C |
| ANISOU | 1320 | CA | ARG | A | 171 | 1259 | 1687 | 1780 | −213 | −477 | 95 | C |
| ATOM | 1321 | C | ARG | A | 171 | 9.689 | 7.394 | 27.045 | 1.00 | 13.29 | | C |
| ANISOU | 1321 | C | ARG | A | 171 | 1213 | 1730 | 2106 | −168 | −333 | 335 | C |
| ATOM | 1322 | O | ARG | A | 171 | 9.319 | 6.791 | 28.055 | 1.00 | 16.18 | | O |
| ANISOU | 1322 | O | ARG | A | 171 | 1550 | 2259 | 2336 | −326 | −525 | 830 | O |
| ATOM | 1323 | CB | ARG | A | 171 | 8.527 | 9.600 | 26.783 | 1.00 | 12.55 | | C |
| ANISOU | 1323 | CB | ARG | A | 171 | 1105 | 1555 | 2108 | −343 | −242 | 398 | C |
| ATOM | 1324 | CG | ARG | A | 171 | 8.500 | 11.093 | 26.967 | 1.00 | 15.21 | | C |
| ANISOU | 1324 | CG | ARG | A | 171 | 1364 | 1602 | 2815 | −253 | −90 | −129 | C |
| ATOM | 1325 | CD | ARG | A | 171 | 8.624 | 11.602 | 28.402 | 1.00 | 21.70 | | C |
| ANISOU | 1325 | CD | ARG | A | 171 | 2818 | 2436 | 2992 | 462 | −417 | −530 | C |
| ATOM | 1326 | NE | ARG | A | 171 | 7.649 | 10.896 | 29.233 | 1.00 | 27.09 | | N |
| ANISOU | 1326 | NE | ARG | A | 171 | 3383 | 4536 | 2375 | 282 | −275 | 25 | N |
| ATOM | 1327 | CZ | ARG | A | 171 | 7.805 | 10.290 | 30.396 | 1.00 | 31.35 | | C |
| ANISOU | 1327 | CZ | ARG | A | 171 | 3474 | 5029 | 3408 | 211 | −1024 | 974 | C |
| ATOM | 1328 | NH1 | ARG | A | 171 | 8.981 | 10.246 | 31.021 | 1.00 | 44.99 | | N |
| ANISOU | 1328 | NH1 | ARG | A | 171 | 4200 | 9388 | 3506 | 2734 | −1707 | −1745 | N |
| ATOM | 1329 | NH2 | ARG | A | 171 | 6.729 | 9.719 | 30.930 | 1.00 | 37.56 | | N |
| ANISOU | 1329 | NH2 | ARG | A | 171 | 5195 | 4202 | 4875 | 607 | 1099 | 1215 | N |
| ATOM | 1330 | N | VAL | A | 172 | 9.950 | 6.793 | 25.882 | 1.00 | 13.25 | | N |
| ANISOU | 1330 | N | VAL | A | 172 | 1303 | 1408 | 2322 | 96 | −622 | 116 | N |
| ATOM | 1331 | CA | VAL | A | 172 | 9.867 | 5.347 | 25.805 | 1.00 | 15.76 | | C |
| ANISOU | 1331 | CA | VAL | A | 172 | 1353 | 1402 | 3233 | −175 | −732 | 278 | C |
| ATOM | 1332 | C | VAL | A | 172 | 10.963 | 4.746 | 26.679 | 1.00 | 19.31 | | C |
| ANISOU | 1332 | C | VAL | A | 172 | 1934 | 1431 | 3971 | 16 | −1137 | 507 | C |
| ATOM | 1333 | O | VAL | A | 172 | 10.722 | 3.851 | 27.492 | 1.00 | 23.02 | | O |
| ANISOU | 1333 | O | VAL | A | 172 | 2395 | 1667 | 4685 | 203 | −1116 | 995 | O |
| ATOM | 1334 | CB | VAL | A | 172 | 9.973 | 4.832 | 24.365 | 1.00 | 15.89 | | C |
| ANISOU | 1334 | CB | VAL | A | 172 | 1270 | 1110 | 3657 | 402 | −637 | −221 | C |
| ATOM | 1335 | CG1 | VAL | A | 172 | 9.944 | 3.301 | 24.385 | 1.00 | 21.67 | | C |
| ANISOU | 1335 | CG1 | VAL | A | 172 | 2586 | 1106 | 4540 | −35 | −1910 | −26 | C |
| ATOM | 1336 | CG2 | VAL | A | 172 | 8.842 | 5.324 | 23.469 | 1.00 | 14.71 | | C |
| ANISOU | 1336 | CG2 | VAL | A | 172 | 1310 | 1136 | 3143 | 102 | −596 | −196 | C |
| ATOM | 1337 | N | LEU | A | 173 | 12.178 | 5.230 | 26.523 | 1.00 | 18.95 | | N |
| ANISOU | 1337 | N | LEU | A | 173 | 1782 | 1420 | 3998 | 53 | −1533 | 370 | N |
| ATOM | 1338 | CA | LEU | A | 173 | 13.286 | 4.752 | 27.335 | 1.00 | 20.44 | | C |
| ANISOU | 1338 | CA | LEU | A | 173 | 2177 | 1721 | 3869 | 99 | −1569 | 812 | C |
| ATOM | 1339 | C | LEU | A | 173 | 13.025 | 4.892 | 28.821 | 1.00 | 24.39 | | C |
| ANISOU | 1339 | C | LEU | A | 173 | 2216 | 3132 | 3918 | 83 | −1251 | 1127 | C |
| ATOM | 1340 | O | LEU | A | 173 | 13.385 | 4.018 | 29.627 | 1.00 | 29.40 | | O |
| ANISOU | 1340 | O | LEU | A | 173 | 3632 | 3425 | 4112 | −225 | −1808 | 1418 | O |
| ATOM | 1341 | CB | LEU | A | 173 | 14.580 | 5.482 | 26.959 | 1.00 | 19.48 | | C |
| ANISOU | 1341 | CB | LEU | A | 173 | 1733 | 2335 | 3334 | 245 | −1438 | 177 | C |
| ATOM | 1342 | CG | LEU | A | 173 | 15.071 | 5.127 | 25.553 | 1.00 | 22.23 | | C |
| ANISOU | 1342 | CG | LEU | A | 173 | 2315 | 2776 | 3356 | 768 | −1440 | 149 | C |
| ATOM | 1343 | CD1 | LEU | A | 173 | 16.237 | 6.025 | 25.168 | 1.00 | 27.54 | | C |
| ANISOU | 1343 | CD1 | LEU | A | 173 | 1883 | 3905 | 4675 | 534 | 198 | −1903 | C |
| ATOM | 1344 | CD2 | LEU | A | 173 | 15.481 | 3.675 | 25.421 | 1.00 | 31.94 | | C |
| ANISOU | 1344 | CD2 | LEU | A | 173 | 3270 | 3243 | 5622 | 1374 | −1785 | −1056 | C |
| ATOM | 1345 | N | ALA | A | 174 | 12.407 | 5.984 | 29.243 | 1.00 | 25.54 | | N |
| ANISOU | 1345 | N | ALA | A | 174 | 3066 | 3092 | 3546 | −215 | −1642 | 267 | N |
| ATOM | 1346 | CA | ALA | A | 174 | 12.136 | 6.192 | 30.674 | 1.00 | 36.86 | | C |
| ANISOU | 1346 | CA | ALA | A | 174 | 5486 | 4927 | 3592 | −95 | −1242 | 116 | C |
| ATOM | 1347 | C | ALA | A | 174 | 11.170 | 5.174 | 31.246 | 1.00 | 43.42 | | C |
| ANISOU | 1347 | C | ALA | A | 174 | 7288 | 5853 | 3356 | −1018 | −184 | −190 | C |
| ATOM | 1348 | O | ALA | A | 174 | 11.090 | 5.330 | 32.450 | 1.00 | 51.16 | | O |
| ANISOU | 1348 | O | ALA | A | 174 | 8258 | 7779 | 3400 | 174 | 455 | −105 | O |
| ATOM | 1349 | CB | ALA | A | 174 | 11.638 | 7.614 | 30.854 | 1.00 | 42.68 | | C |
| ANISOU | 1349 | CB | ALA | A | 174 | 8895 | 4983 | 2338 | 330 | −2303 | −823 | C |
| ATOM | 1350 | N | THR | A | 175 | 10.560 | 4.336 | 30.423 | 1.00 | 52.74 | | N |
| ANISOU | 1350 | N | THR | A | 175 | 9521 | 5255 | 5262 | −1963 | −2220 | 790 | N |
| ATOM | 1351 | CA | THR | A | 175 | 10.418 | 2.903 | 30.648 | 1.00 | 55.30 | | C |
| ANISOU | 1351 | CA | THR | A | 175 | 9449 | 5100 | 6464 | −1002 | −2145 | 955 | C |
| ATOM | 1352 | C | THR | A | 175 | 11.398 | 2.065 | 29.848 | 1.00 | 59.59 | | C |
| ANISOU | 1352 | C | THR | A | 175 | 8606 | 6421 | 7613 | −168 | −2515 | 901 | C |
| ATOM | 1353 | O | THR | A | 175 | 12.104 | 1.194 | 30.408 | 1.00 | 59.63 | | O |
| ANISOU | 1353 | O | THR | A | 175 | 8247 | 6468 | 7943 | −755 | −1922 | 2112 | O |
| ATOM | 1354 | CB | THR | A | 175 | 8.972 | 2.513 | 30.290 | 1.00 | 49.66 | | C |
| ANISOU | 1354 | CB | THR | A | 175 | 8960 | 3579 | 6331 | −854 | −1175 | 1310 | C |
| ATOM | 1355 | OG1 | THR | A | 175 | 8.309 | 2.138 | 31.504 | 1.00 | 53.06 | | O |
| ANISOU | 1355 | OG1 | THR | A | 175 | 9702 | 5181 | 5280 | −837 | −2528 | 2672 | O |
| ATOM | 1356 | CG2 | THR | A | 175 | 8.910 | 1.331 | 29.342 | 1.00 | 61.20 | | C |
| ANISOU | 1356 | CG2 | THR | A | 175 | 11231 | 4734 | 7289 | −1687 | −1778 | 288 | C |
| TER | 1357 | | THR | A | 175 | | | | | | | |
| ATOM | 1358 | N | SER | B | 5 | −13.437 | −8.233 | 6.257 | 1.00 | 72.06 | | N |
| ANISOU | 1358 | N | SER | B | 5 | 4733 | 12677 | 9970 | −606 | 311 | −1701 | N |
| ATOM | 1359 | CA | SER | B | 5 | −14.870 | −8.048 | 6.035 | 1.00 | 60.85 | | C |
| ANISOU | 1359 | CA | SER | B | 5 | 4852 | 8778 | 9489 | 310 | 1077 | −1285 | C |
| ATOM | 1360 | C | SER | B | 5 | −15.582 | −9.335 | 5.627 | 1.00 | 56.03 | | C |
| ANISOU | 1360 | C | SER | B | 5 | 4190 | 9011 | 8090 | −579 | 2367 | −214 | C |
| ATOM | 1361 | O | SER | B | 5 | −16.798 | −9.451 | 5.939 | 1.00 | 76.67 | | O |
| ANISOU | 1361 | O | SER | B | 5 | 3972 | 13615 | 11544 | −808 | 2677 | −4034 | O |
| ATOM | 1362 | CB | SER | B | 5 | −15.094 | −6.966 | 4.972 | 1.00 | 65.22 | | C |
| ANISOU | 1362 | CB | SER | B | 5 | 6553 | 8196 | 10031 | −803 | 1083 | −1019 | C |
| ATOM | 1363 | OG | SER | B | 5 | −16.474 | −6.760 | 4.732 | 1.00 | 69.36 | | O |
| ANISOU | 1363 | OG | SER | B | 5 | 7528 | 6822 | 12002 | −80 | −1384 | −1524 | O |
| ATOM | 1364 | N | SER | B | 6 | −14.983 | −10.276 | 4.982 | 1.00 | 44.40 | | N |
| ANISOU | 1364 | N | SER | B | 6 | 3139 | 7261 | 6469 | −1303 | 2126 | 1162 | N |
| ATOM | 1365 | CA | SER | B | 6 | −13.796 | −10.831 | 4.411 | 1.00 | 36.44 | | C |
| ANISOU | 1365 | CA | SER | B | 6 | 2505 | 6539 | 4800 | −1575 | 1087 | 1070 | C |
| ATOM | 1366 | C | SER | B | 6 | −12.850 | −9.803 | 3.775 | 1.00 | 27.26 | | C |
| ANISOU | 1366 | C | SER | B | 6 | 1512 | 5311 | 3534 | −613 | 67 | 1233 | C |
| ATOM | 1367 | O | SER | B | 6 | −13.258 | −9.077 | 2.866 | 1.00 | 25.56 | | O |
| ANISOU | 1367 | O | SER | B | 6 | 1562 | 4235 | 3914 | −332 | −882 | 421 | O |
| ATOM | 1368 | CB | SER | B | 6 | −14.049 | −11.825 | 3.256 | 1.00 | 40.51 | | C |
| ANISOU | 1368 | CB | SER | B | 6 | 3979 | 5464 | 5951 | −1541 | 369 | 985 | C |
| ATOM | 1369 | OG | SER | B | 6 | −13.270 | −12.994 | 3.420 | 1.00 | 55.54 | | O |
| ANISOU | 1369 | OG | SER | B | 6 | 5481 | 8932 | 6691 | 1760 | 808 | −402 | O |
| ATOM | 1370 | N | LEU | B | 7 | −11.610 | −9.832 | 4.258 | 1.00 | 20.47 | | N |
| ANISOU | 1370 | N | LEU | B | 7 | 1645 | 3877 | 2255 | −201 | 113 | 812 | N |
| ATOM | 1371 | CA | LEU | B | 7 | −10.551 | −8.883 | 3.944 | 1.00 | 16.91 | | C |
| ANISOU | 1371 | CA | LEU | B | 7 | 1470 | 2973 | 1982 | 25 | 292 | −283 | C |
| ATOM | 1372 | C | LEU | B | 7 | −9.435 | −9.575 | 3.171 | 1.00 | 14.60 | | C |
| ANISOU | 1372 | C | LEU | B | 7 | 1653 | 1997 | 1896 | 173 | 225 | 196 | C |
| ATOM | 1373 | O | LEU | B | 7 | −8.826 | −10.536 | 3.627 | 1.00 | 20.37 | | O |
| ANISOU | 1373 | O | LEU | B | 7 | 2709 | 2551 | 2479 | 711 | −399 | 262 | O |
| ATOM | 1374 | CB | LEU | B | 7 | −10.048 | −8.307 | 5.272 | 1.00 | 19.12 | | C |
| ANISOU | 1374 | CB | LEU | B | 7 | 3123 | 2508 | 1632 | 384 | −23 | 134 | C |
| ATOM | 1375 | CG | LEU | B | 7 | −9.029 | −7.186 | 5.196 | 1.00 | 21.85 | | C |
| ANISOU | 1375 | CG | LEU | B | 7 | 2588 | 3285 | 2428 | 102 | −1176 | −135 | C |
| ATOM | 1376 | CD1 | LEU | B | 7 | −9.640 | −5.904 | 4.664 | 1.00 | 20.07 | | C |
| ANISOU | 1376 | CD1 | LEU | B | 7 | 1999 | 2863 | 2764 | −520 | −270 | 415 | C |
| ATOM | 1377 | CD2 | LEU | B | 7 | −8.436 | −6.852 | 6.545 | 1.00 | 33.64 | | C |
| ANISOU | 1377 | CD2 | LEU | B | 7 | 5798 | 4489 | 2494 | −326 | −1952 | −154 | C |
| ATOM | 1378 | N | PHE | B | 8 | −9.190 | −9.105 | 1.973 | 1.00 | 12.22 | | N |
| ANISOU | 1378 | N | PHE | B | 8 | 1195 | 2030 | 1418 | −223 | −157 | −334 | N |
| ATOM | 1379 | CA | PHE | B | 8 | −8.169 | −9.632 | 1.065 | 1.00 | 12.36 | | C |
| ANISOU | 1379 | CA | PHE | B | 8 | 963 | 1873 | 1862 | −95 | −37 | −111 | C |
| ATOM | 1380 | C | PHE | B | 8 | −7.014 | −8.649 | 1.060 | 1.00 | 12.22 | | C |
| ANISOU | 1380 | C | PHE | B | 8 | 927 | 1506 | 2211 | 41 | −136 | −673 | C |
| ATOM | 1381 | O | PHE | B | 8 | −7.236 | −7.494 | 0.692 | 1.00 | 14.01 | | O |
| ANISOU | 1381 | O | PHE | B | 8 | 914 | 1543 | 2866 | 49 | −402 | −551 | O |
| ATOM | 1382 | CB | PHE | B | 8 | −8.734 | −9.734 | −0.331 | 1.00 | 14.65 | | C |
| ANISOU | 1382 | CB | PHE | B | 8 | 1335 | 2465 | 1768 | −546 | 131 | −859 | C |
| ATOM | 1383 | CG | PHE | B | 8 | −9.823 | −10.797 | −0.483 | 1.00 | 15.18 | | C |
| ANISOU | 1383 | CG | PHE | B | 8 | 1638 | 2360 | 1769 | −749 | 422 | −586 | C |
| ATOM | 1384 | CD1 | PHE | B | 8 | −11.112 | −10.532 | −0.080 | 1.00 | 15.95 | | C |
| ANISOU | 1384 | CD1 | PHE | B | 8 | 1543 | 2585 | 1934 | −698 | 250 | −429 | C |
| ATOM | 1385 | CD2 | PHE | B | 8 | −9.533 | −12.021 | −1.025 | 1.00 | 20.86 | | C |
| ANISOU | 1385 | CD2 | PHE | B | 8 | 1953 | 2590 | 3381 | −818 | 461 | −1232 | C |
| ATOM | 1386 | CE1 | PHE | B | 8 | −12.066 | −11.505 | −0.192 | 1.00 | 17.17 | | C |
| ANISOU | 1386 | CE1 | PHE | B | 8 | 1325 | 2608 | 2592 | −549 | −125 | −443 | C |
| ATOM | 1387 | CE2 | PHE | B | 8 | −10.513 | −12.997 | −1.170 | 1.00 | 25.16 | | C |
| ANISOU | 1387 | CE2 | PHE | B | 8 | 1965 | 3347 | 4248 | −1059 | 514 | −2186 | C |
| ATOM | 1388 | CZ | PHE | B | 8 | −11.813 | −12.729 | −0.773 | 1.00 | 17.72 | | C |
| ANISOU | 1388 | CZ | PHE | B | 8 | 1651 | 2302 | 2778 | −467 | −181 | −215 | C |
| ATOM | 1389 | N | LYS | B | 9 | −5.818 | −9.090 | 1.409 | 1.00 | 9.90 | | N |
| ANISOU | 1389 | N | LYS | B | 9 | 1033 | 1490 | 1239 | −150 | −131 | −83 | N |
| ATOM | 1390 | CA | LYS | B | 9 | −4.615 | −8.277 | 1.431 | 1.00 | 9.92 | | C |
| ANISOU | 1390 | CA | LYS | B | 9 | 930 | 1437 | 1403 | −49 | −85 | −197 | C |
| ATOM | 1391 | C | LYS | B | 9 | −3.889 | −8.464 | 0.099 | 1.00 | 9.78 | | C |
| ANISOU | 1391 | C | LYS | B | 9 | 722 | 1630 | 1366 | −5 | −177 | −351 | C |
| ATOM | 1392 | O | LYS | B | 9 | −3.518 | −9.580 | −0.255 | 1.00 | 10.44 | | O |
| ANISOU | 1392 | O | LYS | B | 9 | 1072 | 1604 | 1292 | 70 | −256 | −290 | O |
| ATOM | 1393 | CB | LYS | B | 9 | −3.756 | −8.612 | 2.646 | 1.00 | 10.06 | | C |
| ANISOU | 1393 | CB | LYS | B | 9 | 936 | 1551 | 1333 | −7 | −71 | −354 | C |
| ATOM | 1394 | CG | LYS | B | 9 | −2.438 | −7.790 | 2.645 | 1.00 | 11.47 | | C |
| ANISOU | 1394 | CG | LYS | B | 9 | 940 | 1864 | 1555 | −90 | −212 | −271 | C |
| ATOM | 1395 | CD | LYS | B | 9 | −1.628 | −8.063 | 3.900 | 1.00 | 12.94 | | C |
| ANISOU | 1395 | CD | LYS | B | 9 | 890 | 2676 | 1350 | 0 | −99 | −349 | C |
| ATOM | 1396 | CE | LYS | B | 9 | −0.276 | −7.359 | 3.826 | 1.00 | 12.14 | | C |
| ANISOU | 1396 | CE | LYS | B | 9 | 797 | 2316 | 1501 | 167 | −155 | −442 | C |
| ATOM | 1397 | NZ | LYS | B | 9 | 0.535 | −7.671 | 5.049 | 1.00 | 14.72 | | N |
| ANISOU | 1397 | NZ | LYS | B | 9 | 991 | 3182 | 1419 | −176 | −185 | −197 | N |
| ATOM | 1398 | N | VAL | B | 10 | −3.723 | −7.366 | −0.649 | 1.00 | 8.91 | | N |
| ANISOU | 1398 | N | VAL | B | 10 | 625 | 1532 | 1230 | −59 | −109 | −489 | N |
| ATOM | 1399 | CA | VAL | B | 10 | −3.054 | −7.364 | −1.925 | 1.00 | 9.52 | | C |
| ANISOU | 1399 | CA | VAL | B | 10 | 724 | 1368 | 1524 | −98 | 128 | −578 | C |
| ATOM | 1400 | C | VAL | B | 10 | −1.855 | −6.426 | −1.839 | 1.00 | 9.23 | | C |
| ANISOU | 1400 | C | VAL | B | 10 | 783 | 1573 | 1152 | −217 | 38 | −694 | C |
| ATOM | 1401 | O | VAL | B | 10 | −1.978 | −5.334 | −1.351 | 1.00 | 13.54 | | O |
| ANISOU | 1401 | O | VAL | B | 10 | 1087 | 1678 | 2382 | −376 | 433 | −1095 | O |
| ATOM | 1402 | CB | VAL | B | 10 | −4.051 | −6.913 | −3.033 | 1.00 | 13.58 | | C |
| ANISOU | 1402 | CB | VAL | B | 10 | 958 | 3037 | 1165 | −553 | −191 | −664 | C |
| ATOM | 1403 | CG1 | VAL | B | 10 | −3.408 | −6.706 | −4.371 | 1.00 | 16.52 | | C |
| ANISOU | 1403 | CG1 | VAL | B | 10 | 2249 | 2629 | 1399 | −313 | 257 | −539 | C |
| ATOM | 1404 | CG2 | VAL | B | 10 | −5.195 | −7.936 | −3.048 | 1.00 | 17.61 | | C |
| ANISOU | 1404 | CG2 | VAL | B | 10 | 1690 | 3157 | 1844 | −1025 | −570 | −418 | C |
| ATOM | 1405 | N | ILE | B | 11 | −0.737 | −6.924 | −2.333 | 1.00 | 9.51 | | N |
| ANISOU | 1405 | N | ILE | B | 11 | 842 | 1656 | 1117 | −199 | 191 | −448 | N |
| ATOM | 1406 | CA | ILE | B | 11 | 0.487 | −6.127 | −2.298 | 1.00 | 10.06 | | C |
| ANISOU | 1406 | CA | ILE | B | 11 | 821 | 1927 | 1077 | −331 | 166 | −294 | C |
| ATOM | 1407 | C | ILE | B | 11 | 0.945 | −5.758 | −3.683 | 1.00 | 8.10 | | C |
| ANISOU | 1407 | C | ILE | B | 11 | 865 | 1279 | 932 | −55 | 90 | −407 | C |
| ATOM | 1408 | O | ILE | B | 11 | 0.881 | −6.596 | −4.585 | 1.00 | 9.48 | | O |
| ANISOU | 1408 | O | ILE | B | 11 | 1229 | 1279 | 1093 | −2 | −4 | −452 | O |
| ATOM | 1409 | CB | ILE | B | 11 | 1.618 | −6.843 | −1.540 | 1.00 | 11.59 | | C |
| ANISOU | 1409 | CB | ILE | B | 11 | 1210 | 2054 | 1140 | −422 | −117 | 22 | C |
| ATOM | 1410 | CG1 | ILE | B | 11 | 2.856 | −5.980 | −1.399 | 1.00 | 14.23 | | C |
| ANISOU | 1410 | CG1 | ILE | B | 11 | 1080 | 3045 | 1283 | −636 | −309 | 207 | C |
| ATOM | 1411 | CG2 | ILE | B | 11 | 1.991 | −8.154 | −2.165 | 1.00 | 14.27 | | C |
| ANISOU | 1411 | CG2 | ILE | B | 11 | 1516 | 2393 | 1511 | 229 | −43 | −79 | C |
| ATOM | 1412 | CD1 | ILE | B | 11 | 3.788 | −6.525 | −0.362 | 1.00 | 16.29 | | C |
| ANISOU | 1412 | CD1 | ILE | B | 11 | 1087 | 3261 | 1839 | −527 | −312 | 613 | C |
| ATOM | 1413 | N | LEU | B | 12 | 1.326 | −4.511 | −3.930 | 1.00 | 9.64 | | N |
| ANISOU | 1413 | N | LEU | B | 12 | 1025 | 1449 | 1188 | −405 | 118 | −465 | N |
| ATOM | 1414 | CA | LEU | B | 12 | 1.875 | −4.083 | −5.190 | 1.00 | 9.03 | | C |
| ANISOU | 1414 | CA | LEU | B | 12 | 1066 | 1268 | 1096 | −91 | −66 | −302 | C |
| ATOM | 1415 | C | LEU | B | 12 | 3.385 | −4.032 | −5.075 | 1.00 | 8.66 | | C |
| ANISOU | 1415 | C | LEU | B | 12 | 1026 | 1507 | 760 | −251 | 11 | 22 | C |
| ATOM | 1416 | O | LEU | B | 12 | 3.874 | −3.381 | −4.139 | 1.00 | 11.99 | | O |
| ANISOU | 1416 | O | LEU | B | 12 | 1148 | 2262 | 1144 | −422 | 55 | −492 | O |
| ATOM | 1417 | CB | LEU | B | 12 | 1.456 | −2.682 | −5.607 | 1.00 | 13.50 | | C |
| ANISOU | 1417 | CB | LEU | B | 12 | 1909 | 1524 | 1697 | 232 | −497 | −94 | C |
| ATOM | 1418 | CG | LEU | B | 12 | 0.124 | −2.429 | −6.202 | 1.00 | 17.62 | | C |
| ANISOU | 1418 | CG | LEU | B | 12 | 2081 | 2431 | 2183 | 886 | −547 | −287 | C |
| ATOM | 1419 | CD1 | LEU | B | 12 | −0.127 | −0.930 | −6.157 | 1.00 | 27.60 | | C |
| ANISOU | 1419 | CD1 | LEU | B | 12 | 2492 | 2298 | 5698 | 674 | −1779 | 163 | C |
| ATOM | 1420 | CD2 | LEU | B | 12 | 0.108 | −2.793 | −7.667 | 1.00 | 23.97 | | C |
| ANISOU | 1420 | CD2 | LEU | B | 12 | 2320 | 4921 | 1868 | −921 | −736 | 34 | C |
| ATOM | 1421 | N | LEU | B | 13 | 4.131 | −4.617 | −5.955 | 1.00 | 8.16 | | N |
| ANISOU | 1421 | N | LEU | B | 13 | 1014 | 1253 | 834 | −242 | −71 | 9 | N |
| ATOM | 1422 | CA | LEU | B | 13 | 5.594 | −4.606 | −5.992 | 1.00 | 9.02 | | C |
| ANISOU | 1422 | CA | LEU | B | 13 | 973 | 1393 | 1061 | −208 | −66 | −155 | C |
| ATOM | 1423 | C | LEU | B | 13 | 6.064 | −4.216 | −7.372 | 1.00 | 9.07 | | C |
| ANISOU | 1423 | C | LEU | B | 13 | 911 | 1474 | 1061 | −428 | 10 | −310 | C |
| ATOM | 1424 | O | LEU | B | 13 | 5.368 | −4.498 | −8.352 | 1.00 | 12.52 | | O |
| ANISOU | 1424 | O | LEU | B | 13 | 1355 | 2336 | 1067 | −950 | −191 | −201 | O |
| ATOM | 1425 | CB | LEU | B | 13 | 6.186 | −5.968 | −5.637 | 1.00 | 10.61 | | C |
| ANISOU | 1425 | CB | LEU | B | 13 | 984 | 1357 | 1691 | −303 | −457 | −120 | C |
| ATOM | 1426 | CG | LEU | B | 13 | 5.870 | −6.504 | −4.240 | 1.00 | 11.95 | | C |
| ANISOU | 1426 | CG | LEU | B | 13 | 1513 | 1425 | 1603 | −110 | −610 | −10 | C |
| ATOM | 1427 | CD1 | LEU | B | 13 | 6.507 | −7.856 | −4.075 | 1.00 | 14.73 | | C |
| ANISOU | 1427 | CD1 | LEU | B | 13 | 1888 | 1470 | 2239 | 90 | −308 | 132 | C |
| ATOM | 1428 | CD2 | LEU | B | 13 | 6.300 | −5.555 | −3.126 | 1.00 | 13.78 | | C |
| ANISOU | 1428 | CD2 | LEU | B | 13 | 2029 | 1485 | 1722 | −699 | −521 | 28 | C |
| ATOM | 1429 | N | GLY | B | 14 | 7.210 | −3.601 | −7.475 | 1.00 | 8.34 | | N |
| ANISOU | 1429 | N | GLY | B | 14 | 781 | 1400 | 986 | −229 | −122 | −193 | N |
| ATOM | 1430 | CA | GLY | B | 14 | 7.781 | −3.229 | −8.743 | 1.00 | 9.81 | | C |
| ANISOU | 1430 | CA | GLY | B | 14 | 850 | 1713 | 1165 | −523 | −9 | −140 | C |
| ATOM | 1431 | C | GLY | B | 14 | 8.731 | −2.060 | −8.610 | 1.00 | 8.78 | | C |
| ANISOU | 1431 | C | GLY | B | 14 | 678 | 1581 | 1076 | −362 | −52 | −281 | C |
| ATOM | 1432 | O | GLY | B | 14 | 8.716 | −1.396 | −7.565 | 1.00 | 9.18 | | O |
| ANISOU | 1432 | O | GLY | B | 14 | 983 | 1379 | 1125 | −227 | −50 | −149 | O |
| ATOM | 1433 | N | ASP | B | 15 | 9.515 | −1.818 | −9.631 | 1.00 | 8.47 | | N |
| ANISOU | 1433 | N | ASP | B | 15 | 718 | 1297 | 1203 | −286 | −22 | −131 | N |
| ATOM | 1434 | CA | ASP | B | 15 | 10.517 | −0.784 | −9.561 | 1.00 | 8.79 | | C |
| ANISOU | 1434 | CA | ASP | B | 15 | 887 | 1264 | 1187 | −380 | −237 | 114 | C |
| ATOM | 1435 | C | ASP | B | 15 | 9.943 | 0.574 | −9.247 | 1.00 | 9.76 | | C |
| ANISOU | 1435 | C | ASP | B | 15 | 939 | 1291 | 1479 | −337 | −315 | 7 | C |
| ATOM | 1436 | O | ASP | B | 15 | 8.799 | 0.897 | −9.555 | 1.00 | 10.41 | | O |
| ANISOU | 1436 | O | ASP | B | 15 | 951 | 1514 | 1490 | −270 | −297 | −204 | O |
| ATOM | 1437 | CB | ASP | B | 15 | 11.296 | −0.710 | −10.889 | 1.00 | 10.45 | | C |
| ANISOU | 1437 | CB | ASP | B | 15 | 798 | 1924 | 1247 | −493 | −106 | 143 | C |
| ATOM | 1438 | CG | ASP | B | 15 | 12.298 | −1.815 | −11.113 | 1.00 | 11.83 | | C |
| ANISOU | 1438 | CG | ASP | B | 15 | 837 | 2048 | 1611 | −433 | −229 | −209 | C |
| ATOM | 1439 | OD1 | ASP | B | 15 | 12.358 | −2.730 | −10.257 | 1.00 | 13.24 | | O |
| ANISOU | 1439 | OD1 | ASP | B | 15 | 1141 | 1826 | 2063 | −335 | −37 | −63 | O |
| ATOM | 1440 | OD2 | ASP | B | 15 | 12.998 | −1.746 | −12.168 | 1.00 | 13.23 | | O |
| ANISOU | 1440 | OD2 | ASP | B | 15 | 1228 | 2209 | 1588 | −273 | −125 | −326 | O |
| ATOM | 1441 | N | GLY | B | 16 | 10.756 | 1.449 | −8.630 | 1.00 | 10.93 | | N |
| ANISOU | 1441 | N | GLY | B | 16 | 926 | 1490 | 1736 | −445 | −148 | −271 | N |
| ATOM | 1442 | CA | GLY | B | 16 | 10.318 | 2.822 | −8.519 | 1.00 | 11.40 | | C |
| ANISOU | 1442 | CA | GLY | B | 16 | 1116 | 1527 | 1689 | −316 | −170 | −356 | C |
| ATOM | 1443 | C | GLY | B | 16 | 9.962 | 3.453 | −9.848 | 1.00 | 12.01 | | C |
| ANISOU | 1443 | C | GLY | B | 16 | 1121 | 1585 | 1856 | −487 | −361 | −202 | C |
| ATOM | 1444 | O | GLY | B | 16 | 10.606 | 3.266 | −10.890 | 1.00 | 13.30 | | O |
| ANISOU | 1444 | O | GLY | B | 16 | 1265 | 1806 | 1982 | −215 | −66 | 186 | O |
| ATOM | 1445 | N | GLY | B | 17 | 8.882 | 4.218 | −9.809 | 1.00 | 10.91 | | N |
| ANISOU | 1445 | N | GLY | B | 17 | 1095 | 1539 | 1511 | −553 | −157 | −73 | N |
| ATOM | 1446 | CA | GLY | B | 17 | 8.453 | 5.004 | −10.955 | 1.00 | 11.34 | | C |
| ANISOU | 1446 | CA | GLY | B | 17 | 1157 | 1451 | 1701 | −541 | −191 | −11 | C |
| ATOM | 1447 | C | GLY | B | 17 | 7.513 | 4.276 | −11.878 | 1.00 | 11.22 | | C |
| ANISOU | 1447 | C | GLY | B | 17 | 1540 | 1192 | 1533 | −343 | −401 | 56 | C |
| ATOM | 1448 | O | GLY | B | 17 | 7.082 | 4.926 | −12.821 | 1.00 | 12.02 | | O |
| ANISOU | 1448 | O | GLY | B | 17 | 1650 | 1397 | 1521 | −209 | −250 | 168 | O |
| ATOM | 1449 | N | VAL | B | 18 | 7.214 | 3.004 | −11.642 | 1.00 | 10.97 | | N |
| ANISOU | 1449 | N | VAL | B | 18 | 1349 | 1287 | 1533 | −375 | −548 | 100 | N |
| ATOM | 1450 | CA | VAL | B | 18 | 6.360 | 2.318 | −12.612 | 1.00 | 9.71 | | C |
| ANISOU | 1450 | CA | VAL | B | 18 | 1029 | 1378 | 1283 | −241 | −244 | −132 | C |
| ATOM | 1451 | C | VAL | B | 18 | 4.901 | 2.726 | −12.531 | 1.00 | 9.41 | | C |
| ANISOU | 1451 | C | VAL | B | 18 | 1092 | 1280 | 1204 | −195 | −248 | 55 | C |
| ATOM | 1452 | O | VAL | B | 18 | 4.124 | 2.461 | −13.476 | 1.00 | 11.17 | | O |
| ANISOU | 1452 | O | VAL | B | 18 | 1140 | 1592 | 1512 | −87 | −467 | 204 | O |
| ATOM | 1453 | CB | VAL | B | 18 | 6.488 | 0.782 | −12.508 | 1.00 | 9.59 | | C |
| ANISOU | 1453 | CB | VAL | B | 18 | 1126 | 1344 | 1175 | −176 | −320 | −107 | C |
| ATOM | 1454 | CG1 | VAL | B | 18 | 7.928 | 0.368 | −12.793 | 1.00 | 12.09 | | C |
| ANISOU | 1454 | CG1 | VAL | B | 18 | 1384 | 1978 | 1233 | 206 | −180 | −418 | C |
| ATOM | 1455 | CG2 | VAL | B | 18 | 6.012 | 0.198 | −11.208 | 1.00 | 10.37 | | C |
| ANISOU | 1455 | CG2 | VAL | B | 18 | 1358 | 1573 | 1010 | −244 | −438 | −138 | C |
| ATOM | 1456 | N | GLY | B | 19 | 4.471 | 3.291 | −11.434 | 1.00 | 11.01 | | N |
| ANISOU | 1456 | N | GLY | B | 19 | 1283 | 1250 | 1649 | −101 | −85 | −89 | N |
| ATOM | 1457 | CA | GLY | B | 19 | 3.133 | 3.719 | −11.200 | 1.00 | 11.09 | | C |
| ANISOU | 1457 | CA | GLY | B | 19 | 1206 | 1378 | 1629 | −124 | −109 | −126 | C |
| ATOM | 1458 | C | GLY | B | 19 | 2.338 | 3.068 | −10.116 | 1.00 | 11.03 | | C |
| ANISOU | 1458 | C | GLY | B | 19 | 1143 | 1488 | 1562 | −6 | −182 | −107 | C |
| ATOM | 1459 | O | GLY | B | 19 | 1.118 | 3.110 | −10.164 | 1.00 | 9.73 | | O |
| ANISOU | 1459 | O | GLY | B | 19 | 1066 | 1139 | 1490 | −118 | −199 | −73 | O |
| ATOM | 1460 | N | LYS | B | 20 | 2.944 | 2.413 | −9.133 | 1.00 | 10.29 | | N |
| ANISOU | 1460 | N | LYS | B | 20 | 1120 | 1297 | 1490 | 83 | −149 | −236 | N |
| ATOM | 1461 | CA | LYS | B | 20 | 2.256 | 1.688 | −8.100 | 1.00 | 10.39 | | C |
| ANISOU | 1461 | CA | LYS | B | 20 | 922 | 1446 | 1582 | 82 | −137 | −157 | C |
| ATOM | 1462 | C | LYS | B | 20 | 1.362 | 2.612 | −7.285 | 1.00 | 9.93 | | C |
| ANISOU | 1462 | C | LYS | B | 20 | 841 | 1450 | 1481 | −54 | −210 | −113 | C |
| ATOM | 1463 | O | LYS | B | 20 | 0.170 | 2.317 | −7.122 | 1.00 | 10.44 | | O |
| ANISOU | 1463 | O | LYS | B | 20 | 836 | 1599 | 1531 | −114 | −131 | −377 | O |
| ATOM | 1464 | CB | LYS | B | 20 | 3.279 | 0.985 | −7.200 | 1.00 | 9.81 | | C |
| ANISOU | 1464 | CB | LYS | B | 20 | 964 | 1179 | 1584 | −138 | −240 | −212 | C |
| ATOM | 1465 | CG | LYS | B | 20 | 4.148 | −0.044 | −7.935 | 1.00 | 10.50 | | C |
| ANISOU | 1465 | CG | LYS | B | 20 | 1054 | 1288 | 1648 | 94 | −328 | −171 | C |
| ATOM | 1466 | CD | LYS | B | 20 | 5.151 | −0.696 | −7.027 | 1.00 | 10.08 | | C |
| ANISOU | 1466 | CD | LYS | B | 20 | 947 | 1207 | 1677 | −135 | −290 | −48 | C |
| ATOM | 1467 | CE | LYS | B | 20 | 6.125 | 0.218 | −6.340 | 1.00 | 11.15 | | C |
| ANISOU | 1467 | CE | LYS | B | 20 | 1203 | 1566 | 1469 | −219 | −312 | −177 | C |
| ATOM | 1468 | NZ | LYS | B | 20 | 6.946 | 1.031 | −7.260 | 1.00 | 11.62 | | N |
| ANISOU | 1468 | NZ | LYS | B | 20 | 1192 | 1493 | 1732 | −396 | −272 | −126 | N |
| ATOM | 1469 | N | SER | B | 21 | 1.892 | 3.717 | −6.792 | 1.00 | 10.57 | | N |
| ANISOU | 1469 | N | SER | B | 21 | 950 | 1426 | 1641 | −148 | −154 | −270 | N |
| ATOM | 1470 | CA | SER | B | 21 | 1.048 | 4.622 | −6.033 | 1.00 | 12.57 | | C |
| ANISOU | 1470 | CA | SER | B | 21 | 1196 | 1854 | 1726 | 85 | −143 | −380 | C |
| ATOM | 1471 | C | SER | B | 21 | −0.100 | 5.143 | −6.853 | 1.00 | 12.92 | | C |
| ANISOU | 1471 | C | SER | B | 21 | 1345 | 1561 | 2003 | 102 | −323 | −354 | C |
| ATOM | 1472 | O | SER | B | 21 | −1.236 | 5.246 | −6.418 | 1.00 | 13.04 | | O |
| ANISOU | 1472 | O | SER | B | 21 | 1288 | 1528 | 2138 | 154 | −375 | −480 | O |
| ATOM | 1473 | CB | SER | B | 21 | 1.906 | 5.812 | −5.517 | 1.00 | 17.53 | | C |
| ANISOU | 1473 | CB | SER | B | 21 | 1703 | 2545 | 2414 | 139 | −437 | −1541 | C |
| ATOM | 1474 | OG | SER | B | 21 | 1.075 | 6.780 | −4.941 | 1.00 | 26.03 | | O |
| ANISOU | 1474 | OG | SER | B | 21 | 2512 | 3156 | 4223 | 733 | −500 | −2176 | O |
| ATOM | 1475 | N | SER | B | 22 | 0.188 | 5.465 | −8.103 | 1.00 | 12.75 | | N |
| ANISOU | 1475 | N | SER | B | 22 | 1413 | 1341 | 2090 | 179 | −352 | −322 | N |
| ATOM | 1476 | CA | SER | B | 22 | −0.838 | 6.015 | −8.970 | 1.00 | 12.54 | | C |
| ANISOU | 1476 | CA | SER | B | 22 | 1304 | 1439 | 2023 | 244 | −226 | −256 | C |
| ATOM | 1477 | C | SER | B | 22 | −1.914 | 4.985 | −9.284 | 1.00 | 12.52 | | C |
| ANISOU | 1477 | C | SER | B | 22 | 1210 | 1471 | 2074 | 172 | −210 | −68 | C |
| ATOM | 1478 | O | SER | B | 22 | −3.080 | 5.366 | −9.370 | 1.00 | 13.71 | | O |
| ANISOU | 1478 | O | SER | B | 22 | 1130 | 1856 | 2222 | 334 | −9 | 209 | O |
| ATOM | 1479 | CB | SER | B | 22 | −0.257 | 6.533 | −10.275 | 1.00 | 15.96 | | C |
| ANISOU | 1479 | CB | SER | B | 22 | 2082 | 1657 | 2325 | −314 | −196 | 116 | C |
| ATOM | 1480 | OG | SER | B | 22 | 0.734 | 7.552 | −10.088 | 1.00 | 14.49 | | O |
| ANISOU | 1480 | OG | SER | B | 22 | 1568 | 1816 | 2122 | −97 | −331 | −116 | O |
| ATOM | 1481 | N | LEU | B | 23 | −1.536 | 3.717 | −9.442 | 1.00 | 10.50 | | N |
| ANISOU | 1481 | N | LEU | B | 23 | 925 | 1613 | 1453 | 128 | −248 | −299 | N |
| ATOM | 1482 | CA | LEU | B | 23 | −2.553 | 2.697 | −9.681 | 1.00 | 11.71 | | C |
| ANISOU | 1482 | CA | LEU | B | 23 | 1128 | 1549 | 1772 | 32 | −383 | −180 | C |
| ATOM | 1483 | C | LEU | B | 23 | −3.465 | 2.518 | −8.490 | 1.00 | 12.42 | | C |
| ANISOU | 1483 | C | LEU | B | 23 | 1084 | 1716 | 1919 | −55 | −247 | −201 | C |
| ATOM | 1484 | O | LEU | B | 23 | −4.671 | 2.426 | −8.572 | 1.00 | 12.33 | | O |
| ANISOU | 1484 | O | LEU | B | 23 | 1063 | 1693 | 1928 | 248 | −331 | −244 | O |
| ATOM | 1485 | CB | LEU | B | 23 | −1.853 | 1.398 | −10.028 | 1.00 | 11.54 | | C |
| ANISOU | 1485 | CB | LEU | B | 23 | 1382 | 1528 | 1475 | 80 | −343 | −124 | C |
| ATOM | 1486 | CG | LEU | B | 23 | −1.317 | 1.315 | −11.446 | 1.00 | 11.74 | | C |
| ANISOU | 1486 | CG | LEU | B | 23 | 1557 | 1467 | 1436 | −132 | −381 | −172 | C |
| ATOM | 1487 | CD1 | LEU | B | 23 | −0.296 | 0.172 | −11.576 | 1.00 | 12.27 | | C |
| ANISOU | 1487 | CD1 | LEU | B | 23 | 1557 | 1585 | 1519 | −67 | −337 | −404 | C |
| ATOM | 1488 | CD2 | LEU | B | 23 | −2.446 | 1.158 | −12.459 | 1.00 | 11.38 | | C |
| ANISOU | 1488 | CD2 | LEU | B | 23 | 1154 | 1767 | 1402 | −278 | −79 | −391 | C |
| ATOM | 1489 | N | MET | B | 24 | −2.848 | 2.448 | −7.299 | 1.00 | 11.93 | | N |
| ANISOU | 1489 | N | MET | B | 24 | 866 | 1692 | 1977 | −3 | −185 | −321 | N |
| ATOM | 1490 | CA | MET | B | 24 | −3.719 | 2.285 | −6.150 | 1.00 | 14.16 | | C |
| ANISOU | 1490 | CA | MET | B | 24 | 1835 | 1708 | 1838 | −214 | 153 | −783 | C |
| ATOM | 1491 | C | MET | B | 24 | −4.633 | 3.470 | −5.918 | 1.00 | 13.81 | | C |
| ANISOU | 1491 | C | MET | B | 24 | 1464 | 2035 | 1750 | −128 | 203 | −513 | C |
| ATOM | 1492 | O | MET | B | 24 | −5.761 | 3.294 | −5.571 | 1.00 | 15.45 | | O |
| ANISOU | 1492 | O | MET | B | 24 | 1273 | 2654 | 1945 | −338 | −81 | −691 | O |
| ATOM | 1493 | CB | MET | B | 24 | −2.857 | 2.199 | −4.967 | 1.00 | 18.35 | | C |
| ANISOU | 1493 | CB | MET | B | 24 | 2345 | 2482 | 2145 | 410 | 37 | −250 | C |
| ATOM | 1494 | CG | MET | B | 24 | −3.070 | 2.319 | −3.538 | 1.00 | 25.28 | | C |
| ANISOU | 1494 | CG | MET | B | 24 | 4124 | 3470 | 2009 | 1183 | −589 | −161 | C |
| ATOM | 1495 | SD | MET | B | 24 | −1.870 | 1.742 | −2.276 | 1.00 | 26.02 | | S |
| ANISOU | 1495 | SD | MET | B | 24 | 3051 | 3738 | 3098 | 164 | −989 | 148 | S |
| ATOM | 1496 | CE | MET | B | 24 | −0.868 | 0.891 | −3.469 | 1.00 | 25.72 | | C |
| ANISOU | 1496 | CE | MET | B | 24 | 1207 | 7010 | 1554 | −229 | −774 | 1231 | C |
| ATOM | 1497 | N | ASN | B | 25 | −4.086 | 4.667 | −6.079 | 1.00 | 13.28 | | N |
| ANISOU | 1497 | N | ASN | B | 25 | 1134 | 1830 | 2081 | 122 | 95 | −568 | N |
| ATOM | 1498 | CA | ASN | B | 25 | −4.899 | 5.860 | −5.885 | 1.00 | 16.34 | | C |
| ANISOU | 1498 | CA | ASN | B | 25 | 1447 | 2156 | 2607 | 342 | −179 | −1017 | C |
| ATOM | 1499 | C | ASN | B | 25 | −5.982 | 5.968 | −6.950 | 1.00 | 16.66 | | C |
| ANISOU | 1499 | C | ASN | B | 25 | 1484 | 2330 | 2516 | 527 | −162 | −794 | C |
| ATOM | 1500 | O | ASN | B | 25 | −7.105 | 6.354 | −6.645 | 1.00 | 16.71 | | O |
| ANISOU | 1500 | O | ASN | B | 25 | 1397 | 2575 | 2378 | 356 | 12 | −695 | O |
| ATOM | 1501 | CB | ASN | B | 25 | −3.987 | 7.097 | −5.800 | 1.00 | 19.82 | | C |
| ANISOU | 1501 | CB | ASN | B | 25 | 1772 | 2114 | 3644 | 296 | 195 | −2010 | C |
| ATOM | 1502 | CG | ASN | B | 25 | −3.246 | 7.075 | −4.472 | 1.00 | 25.70 | | C |
| ANISOU | 1502 | CG | ASN | B | 25 | 2128 | 3366 | 4272 | 325 | −468 | −2581 | C |
| ATOM | 1503 | OD1 | ASN | B | 25 | −3.823 | 6.972 | −3.381 | 1.00 | 35.19 | | O |
| ANISOU | 1503 | OD1 | ASN | B | 25 | 4163 | 5312 | 3895 | −1608 | −799 | −406 | O |
| ATOM | 1504 | ND2 | ASN | B | 25 | −1.948 | 7.254 | −4.495 | 1.00 | 43.37 | | N |
| ANISOU | 1504 | ND2 | ASN | B | 25 | 1797 | 7970 | 6711 | 1188 | −674 | −4843 | N |
| ATOM | 1505 | N | ARG | B | 26 | −5.679 | 5.587 | −8.191 | 1.00 | 15.14 | | N |
| ANISOU | 1505 | N | ARG | B | 26 | 1058 | 2246 | 2448 | 226 | −45 | −612 | N |
| ATOM | 1506 | CA | ARG | B | 26 | −6.656 | 5.581 | −9.241 | 1.00 | 13.13 | | C |
| ANISOU | 1506 | CA | ARG | B | 26 | 1272 | 1455 | 2261 | 391 | −83 | −96 | C |
| ATOM | 1507 | C | ARG | B | 26 | −7.813 | 4.630 | −8.920 | 1.00 | 12.01 | | C |
| ANISOU | 1507 | C | ARG | B | 26 | 1013 | 1773 | 1779 | 385 | −128 | −107 | C |
| ATOM | 1508 | O | ARG | B | 26 | −8.991 | 4.942 | −9.064 | 1.00 | 14.31 | | O |
| ANISOU | 1508 | O | ARG | B | 26 | 1103 | 2332 | 2003 | 458 | −375 | −253 | O |
| ATOM | 1509 | CB | ARG | B | 26 | −6.061 | 5.130 | −10.562 | 1.00 | 17.33 | | C |
| ANISOU | 1509 | CB | ARG | B | 26 | 1357 | 3063 | 2164 | 124 | 205 | −38 | C |
| ATOM | 1510 | CG | ARG | B | 26 | −7.111 | 5.092 | −11.683 | 1.00 | 20.67 | | C |
| ANISOU | 1510 | CG | ARG | B | 26 | 2059 | 3807 | 1988 | 545 | 15 | 11 | C |
| ATOM | 1511 | CD | ARG | B | 26 | −7.080 | 6.576 | −12.105 | 1.00 | 34.30 | | C |
| ANISOU | 1511 | CD | ARG | B | 26 | 5373 | 3774 | 3884 | 1252 | −1378 | 257 | C |
| ATOM | 1512 | NE | ARG | B | 26 | −8.317 | 7.008 | −12.621 | 1.00 | 36.24 | | N |
| ANISOU | 1512 | NE | ARG | B | 26 | 5693 | 3566 | 4510 | 1102 | −2141 | −253 | N |
| ATOM | 1513 | CZ | ARG | B | 26 | −8.527 | 7.983 | −13.489 | 1.00 | 31.66 | | C |
| ANISOU | 1513 | CZ | ARG | B | 26 | 4343 | 3703 | 3984 | 799 | −1945 | −317 | C |
| ATOM | 1514 | NH1 | ARG | B | 26 | −7.539 | 8.688 | −13.991 | 1.00 | 29.97 | | N |
| ANISOU | 1514 | NH1 | ARG | B | 26 | 3444 | 4464 | 3478 | 1918 | −877 | −522 | N |
| ATOM | 1515 | NH2 | ARG | B | 26 | −9.784 | 8.185 | −13.809 | 1.00 | 34.59 | | N |
| ANISOU | 1515 | NH2 | ARG | B | 26 | 3835 | 3490 | 5819 | 1237 | −864 | 321 | N |
| ATOM | 1516 | N | TYR | B | 27 | −7.455 | 3.442 | −8.445 | 1.00 | 12.66 | | N |
| ANISOU | 1516 | N | TYR | B | 27 | 1189 | 1684 | 1937 | 187 | −267 | −86 | N |
| ATOM | 1517 | CA | TYR | B | 27 | −8.461 | 2.434 | −8.138 | 1.00 | 12.18 | | C |
| ANISOU | 1517 | CA | TYR | B | 27 | 1349 | 1912 | 1366 | 36 | −237 | −192 | C |
| ATOM | 1518 | C | TYR | B | 27 | −9.344 | 2.908 | −7.003 | 1.00 | 13.28 | | C |
| ANISOU | 1518 | C | TYR | B | 27 | 1220 | 2323 | 1501 | 116 | −299 | −494 | C |
| ATOM | 1519 | O | TYR | B | 27 | −10.570 | 2.799 | −7.056 | 1.00 | 15.26 | | O |
| ANISOU | 1519 | O | TYR | B | 27 | 1201 | 2777 | 1821 | 305 | −372 | −713 | O |
| ATOM | 1520 | CB | TYR | B | 27 | −7.810 | 1.098 | −7.770 | 1.00 | 13.15 | | C |
| ANISOU | 1520 | CB | TYR | B | 27 | 1501 | 1826 | 1669 | 46 | −103 | −130 | C |
| ATOM | 1521 | CG | TYR | B | 27 | −8.790 | −0.004 | −7.466 | 1.00 | 12.02 | | C |
| ANISOU | 1521 | CG | TYR | B | 27 | 1340 | 1899 | 1328 | 50 | −171 | −190 | C |
| ATOM | 1522 | CD1 | TYR | B | 27 | −9.853 | −0.306 | −8.328 | 1.00 | 14.76 | | C |
| ANISOU | 1522 | CD1 | TYR | B | 27 | 1375 | 2319 | 1914 | 67 | −563 | 170 | C |
| ATOM | 1523 | CD2 | TYR | B | 27 | −8.667 | −0.787 | −6.355 | 1.00 | 12.01 | | C |
| ANISOU | 1523 | CD2 | TYR | B | 27 | 1020 | 1854 | 1691 | 250 | −277 | 5 | C |
| ATOM | 1524 | CE1 | TYR | B | 27 | −10.751 | −1.310 | −8.078 | 1.00 | 14.32 | | C |
| ANISOU | 1524 | CE1 | TYR | B | 27 | 1416 | 2575 | 1449 | −151 | −417 | −229 | C |
| ATOM | 1525 | CE2 | TYR | B | 27 | −9.538 | −1.790 | −6.086 | 1.00 | 12.57 | | C |
| ANISOU | 1525 | CE2 | TYR | B | 27 | 1484 | 2024 | 1269 | −110 | −237 | −160 | C |
| ATOM | 1526 | CZ | TYR | B | 27 | −10.586 | −2.081 | −6.935 | 1.00 | 13.01 | | C |
| ANISOU | 1526 | CZ | TYR | B | 27 | 1083 | 2194 | 1667 | 44 | −159 | −204 | C |
| ATOM | 1527 | OH | TYR | B | 27 | −11.469 | −3.082 | −6.624 | 1.00 | 16.22 | | O |
| ANISOU | 1527 | OH | TYR | B | 27 | 1609 | 2473 | 2082 | −454 | −70 | −342 | O |
| ATOM | 1528 | N | VAL | B | 28 | −8.761 | 3.437 | −5.939 | 1.00 | 14.57 | | N |
| ANISOU | 1528 | N | VAL | B | 28 | 1194 | 2689 | 1653 | −197 | −99 | −707 | N |
| ATOM | 1529 | CA | VAL | B | 28 | −9.529 | 3.759 | −4.737 | 1.00 | 14.65 | | C |
| ANISOU | 1529 | CA | VAL | B | 28 | 1576 | 2530 | 1461 | 29 | −178 | −540 | C |
| ATOM | 1530 | C | VAL | B | 28 | −10.252 | 5.089 | −4.855 | 1.00 | 15.26 | | C |
| ANISOU | 1530 | C | VAL | B | 28 | 1214 | 2966 | 1619 | 275 | −100 | −385 | C |
| ATOM | 1531 | O | VAL | B | 28 | −11.370 | 5.200 | −4.365 | 1.00 | 20.42 | | O |
| ANISOU | 1531 | O | VAL | B | 28 | 1422 | 3988 | 2348 | 402 | 329 | −373 | O |
| ATOM | 1532 | CB | VAL | B | 28 | −8.580 | 3.734 | −3.532 | 1.00 | 13.76 | | C |
| ANISOU | 1532 | CB | VAL | B | 28 | 1629 | 2075 | 1526 | −143 | −249 | −306 | C |
| ATOM | 1533 | CG1 | VAL | B | 28 | −9.369 | 4.204 | −2.315 | 1.00 | 15.85 | | C |
| ANISOU | 1533 | CG1 | VAL | B | 28 | 1861 | 2794 | 1369 | 265 | −345 | −294 | C |
| ATOM | 1534 | CG2 | VAL | B | 28 | −8.051 | 2.329 | −3.343 | 1.00 | 15.93 | | C |
| ANISOU | 1534 | CG2 | VAL | B | 28 | 1712 | 2131 | 2210 | −117 | −244 | −194 | C |
| ATOM | 1535 | N | THR | B | 29 | −9.653 | 6.076 | −5.490 | 1.00 | 16.02 | | N |
| ANISOU | 1535 | N | THR | B | 29 | 1269 | 2579 | 2238 | 499 | 84 | −466 | N |
| ATOM | 1536 | CA | THR | B | 29 | −10.267 | 7.393 | −5.462 | 1.00 | 20.98 | | C |
| ANISOU | 1536 | CA | THR | B | 29 | 2098 | 2675 | 3200 | 780 | −446 | −632 | C |
| ATOM | 1537 | C | THR | B | 29 | −10.775 | 7.846 | −6.823 | 1.00 | 21.46 | | C |
| ANISOU | 1537 | C | THR | B | 29 | 2019 | 2919 | 3218 | 866 | −346 | −325 | C |
| ATOM | 1538 | O | THR | B | 29 | −11.421 | 8.886 | −6.821 | 1.00 | 24.83 | | O |
| ANISOU | 1538 | O | THR | B | 29 | 2852 | 2993 | 3589 | 1133 | −246 | −197 | O |
| ATOM | 1539 | CB | THR | B | 29 | −9.325 | 8.515 | −4.956 | 1.00 | 24.02 | | C |
| ANISOU | 1539 | CB | THR | B | 29 | 2223 | 2819 | 4082 | 868 | −510 | −1288 | C |
| ATOM | 1540 | OG1 | THR | B | 29 | −8.244 | 8.700 | −5.867 | 1.00 | 26.83 | | O |
| ANISOU | 1540 | OG1 | THR | B | 29 | 2111 | 2523 | 5561 | 828 | −19 | −1002 | O |
| ATOM | 1541 | CG2 | THR | B | 29 | −8.755 | 8.086 | −3.629 | 1.00 | 29.51 | | C |
| ANISOU | 1541 | CG2 | THR | B | 29 | 3011 | 3623 | 4578 | 775 | −1412 | −1392 | C |
| ATOM | 1542 | N | ASN | B | 30 | −10.461 | 7.122 | −7.868 | 1.00 | 22.81 | | N |
| ANISOU | 1542 | N | ASN | B | 30 | 2107 | 3373 | 3187 | 1055 | −449 | −411 | N |
| ATOM | 1543 | CA | ASN | B | 30 | −10.734 | 7.426 | −9.275 | 1.00 | 22.99 | | C |
| ANISOU | 1543 | CA | ASN | B | 30 | 1760 | 3856 | 3118 | 1098 | 66 | −131 | C |
| ATOM | 1544 | C | ASN | B | 30 | −10.152 | 8.766 | −9.691 | 1.00 | 24.90 | | C |
| ANISOU | 1544 | C | ASN | B | 30 | 2486 | 3533 | 3441 | 1263 | −77 | −188 | C |
| ATOM | 1545 | O | ASN | B | 30 | −10.752 | 9.506 | −10.472 | 1.00 | 27.66 | | O |
| ANISOU | 1545 | O | ASN | B | 30 | 2488 | 4187 | 3834 | 1308 | −9 | 361 | O |
| ATOM | 1546 | CB | ASN | B | 30 | −12.225 | 7.388 | −9.595 | 1.00 | 27.10 | | C |
| ANISOU | 1546 | CB | ASN | B | 30 | 2013 | 4591 | 3692 | 1262 | −574 | −152 | C |
| ATOM | 1547 | CG | ASN | B | 30 | −12.435 | 7.349 | −11.103 | 1.00 | 32.05 | | C |
| ANISOU | 1547 | CG | ASN | B | 30 | 3044 | 5405 | 3730 | 1925 | −863 | −322 | C |
| ATOM | 1548 | OD1 | ASN | B | 30 | −11.619 | 6.797 | −11.848 | 1.00 | 35.61 | | O |
| ANISOU | 1548 | OD1 | ASN | B | 30 | 3518 | 6247 | 3767 | 2056 | −449 | −315 | O |
| ATOM | 1549 | ND2 | ASN | B | 30 | −13.523 | 7.911 | −11.614 | 1.00 | 36.73 | | N |
| ANISOU | 1549 | ND2 | ASN | B | 30 | 3239 | 6664 | 4051 | 2138 | −813 | 561 | N |
| ATOM | 1550 | N | LYS | B | 31 | −8.970 | 9.065 | −9.168 | 1.00 | 23.94 | | N |
| ANISOU | 1550 | N | LYS | B | 31 | 2744 | 3644 | 2706 | 618 | 12 | 377 | N |
| ATOM | 1551 | CA | LYS | B | 31 | −8.280 | 10.325 | −9.463 | 1.00 | 27.09 | | C |
| ANISOU | 1551 | CA | LYS | B | 31 | 2817 | 3234 | 4243 | 1017 | 770 | 123 | C |
| ATOM | 1552 | C | LYS | B | 31 | −6.855 | 10.011 | −9.915 | 1.00 | 23.63 | | C |
| ANISOU | 1552 | C | LYS | B | 31 | 2302 | 2884 | 3791 | 1156 | −96 | 705 | C |
| ATOM | 1553 | O | LYS | B | 31 | −6.322 | 9.016 | −9.438 | 1.00 | 26.26 | | O |
| ANISOU | 1553 | O | LYS | B | 31 | 3265 | 2448 | 4264 | 1095 | −1034 | 236 | O |
| ATOM | 1554 | CB | LYS | B | 31 | −8.193 | 11.251 | −8.252 | 1.00 | 33.32 | | C |
| ANISOU | 1554 | CB | LYS | B | 31 | 5672 | 2508 | 4482 | 939 | 1235 | 200 | C |
| ATOM | 1555 | CG | LYS | B | 31 | −9.513 | 11.636 | −7.582 | 1.00 | 46.26 | | C |
| ANISOU | 1555 | CG | LYS | B | 31 | 6504 | 5072 | 6000 | 1499 | 1747 | −1686 | C |
| ATOM | 1556 | CD | LYS | B | 31 | −9.322 | 11.919 | −6.095 | 1.00 | 57.71 | | C |
| ANISOU | 1556 | CD | LYS | B | 31 | 8298 | 7102 | 6525 | 2847 | 1566 | −3369 | C |
| ATOM | 1557 | CE | LYS | B | 31 | −10.607 | 11.982 | −5.276 | 1.00 | 58.95 | | C |
| ANISOU | 1557 | CE | LYS | B | 31 | 8433 | 7751 | 6212 | 3859 | 1481 | −3777 | C |
| ATOM | 1558 | NZ | LYS | B | 31 | −10.425 | 12.579 | −3.918 | 1.00 | 53.03 | | N |
| ANISOU | 1558 | NZ | LYS | B | 31 | 8781 | 5548 | 5820 | 2137 | 1478 | −2679 | N |
| ATOM | 1559 | N | PHE | B | 32 | −6.286 | 10.824 | −10.765 | 1.00 | 23.69 | | N |
| ANISOU | 1559 | N | PHE | B | 32 | 2337 | 3132 | 3531 | 955 | 91 | 385 | N |
| ATOM | 1560 | CA | PHE | B | 32 | −4.880 | 10.740 | −11.159 | 1.00 | 22.79 | | C |
| ANISOU | 1560 | CA | PHE | B | 32 | 2352 | 2423 | 3886 | 1099 | 157 | 16 | C |
| ATOM | 1561 | C | PHE | B | 32 | −4.349 | 12.165 | −11.076 | 1.00 | 22.87 | | C |
| ANISOU | 1561 | C | PHE | B | 32 | 2648 | 2451 | 3591 | 989 | 237 | 159 | C |
| ATOM | 1562 | O | PHE | B | 32 | −4.886 | 13.087 | −11.692 | 1.00 | 32.57 | | O |
| ANISOU | 1562 | O | PHE | B | 32 | 5465 | 2528 | 4383 | 1791 | −107 | 319 | O |
| ATOM | 1563 | CB | PHE | B | 32 | −4.678 | 10.152 | −12.545 | 1.00 | 21.95 | | C |
| ANISOU | 1563 | CB | PHE | B | 32 | 1755 | 2836 | 3749 | 480 | −82 | −8 | C |
| ATOM | 1564 | CG | PHE | B | 32 | −3.254 | 10.234 | −13.061 | 1.00 | 18.96 | | C |
| ANISOU | 1564 | CG | PHE | B | 32 | 1918 | 1693 | 3595 | 275 | 9 | −4 | C |
| ATOM | 1565 | CD1 | PHE | B | 32 | −2.384 | 9.221 | −12.700 | 1.00 | 20.23 | | C |
| ANISOU | 1565 | CD1 | PHE | B | 32 | 1761 | 2621 | 3307 | 477 | −331 | 405 | C |
| ATOM | 1566 | CD2 | PHE | B | 32 | −2.797 | 11.263 | −13.855 | 1.00 | 19.97 | | C |
| ANISOU | 1566 | CD2 | PHE | B | 32 | 2328 | 2415 | 2846 | −60 | −657 | 277 | C |
| ATOM | 1567 | CE1 | PHE | B | 32 | −1.074 | 9.238 | −13.132 | 1.00 | 18.33 | | C |
| ANISOU | 1567 | CE1 | PHE | B | 32 | 2100 | 2482 | 2380 | 590 | 9 | 235 | C |
| ATOM | 1568 | CE2 | PHE | B | 32 | −1.497 | 11.258 | −14.304 | 1.00 | 20.01 | | C |
| ANISOU | 1568 | CE2 | PHE | B | 32 | 2652 | 2413 | 2539 | −36 | −209 | 442 | C |
| ATOM | 1569 | CZ | PHE | B | 32 | −0.626 | 10.242 | −13.947 | 1.00 | 19.49 | | C |
| ANISOU | 1569 | CZ | PHE | B | 32 | 2661 | 2123 | 2621 | 113 | −59 | −40 | C |
| ATOM | 1570 | N | ASP | B | 33 | −3.316 | 12.381 | −10.295 | 1.00 | 33.28 | | N |
| ANISOU | 1570 | N | ASP | B | 33 | 3874 | 3394 | 5377 | 1280 | −942 | −1905 | N |
| ATOM | 1571 | CA | ASP | B | 33 | −2.871 | 13.759 | −10.069 | 1.00 | 50.30 | | C |
| ANISOU | 1571 | CA | ASP | B | 33 | 7772 | 4267 | 7071 | −335 | −803 | −2919 | C |
| ATOM | 1572 | C | ASP | B | 33 | −1.379 | 13.833 | −10.304 | 1.00 | 56.70 | | C |
| ANISOU | 1572 | C | ASP | B | 33 | 8027 | 4662 | 8854 | −1922 | −441 | −2536 | C |
| ATOM | 1573 | O | ASP | B | 33 | −0.573 | 13.280 | −9.554 | 1.00 | 53.02 | | O |
| ANISOU | 1573 | O | ASP | B | 33 | 6907 | 5958 | 7280 | −53 | 1101 | −3784 | O |
| ATOM | 1574 | CB | ASP | B | 33 | −3.289 | 14.192 | −8.672 | 1.00 | 59.95 | | C |
| ANISOU | 1574 | CB | ASP | B | 33 | 8906 | 6371 | 7503 | 473 | −1379 | −4460 | C |
| ATOM | 1575 | CG | ASP | B | 33 | −2.478 | 13.606 | −7.536 | 1.00 | 70.40 | | C |
| ANISOU | 1575 | CG | ASP | B | 33 | 11139 | 8371 | 7239 | 321 | −2052 | −3912 | C |
| ATOM | 1576 | OD1 | ASP | B | 33 | −2.371 | 12.363 | −7.436 | 1.00 | 75.27 | | O |
| ANISOU | 1576 | OD1 | ASP | B | 33 | 10056 | 8863 | 9679 | 4002 | −1401 | −4261 | O |
| ATOM | 1577 | OD2 | ASP | B | 33 | −1.927 | 14.401 | −6.742 | 1.00 | 84.10 | | O |
| ANISOU | 1577 | OD2 | ASP | B | 33 | 15363 | 11934 | 4660 | −2805 | −1518 | −3566 | O |
| ATOM | 1578 | N | THR | B | 34 | −0.969 | 14.523 | −11.362 | 1.00 | 65.46 | | N |
| ANISOU | 1578 | N | THR | B | 34 | 9692 | 5537 | 9642 | −1920 | 279 | −1887 | N |
| ATOM | 1579 | CA | THR | B | 34 | 0.447 | 14.654 | −11.685 | 1.00 | 67.74 | | C |
| ANISOU | 1579 | CA | THR | B | 34 | 9948 | 5981 | 9810 | −2396 | 776 | −1854 | C |
| ATOM | 1580 | C | THR | B | 34 | 1.125 | 13.284 | −11.773 | 1.00 | 73.07 | | C |
| ANISOU | 1580 | C | THR | B | 34 | 10065 | 7048 | 10649 | −1352 | 425 | −2893 | C |
| ATOM | 1581 | O | THR | B | 34 | 0.754 | 12.416 | −12.580 | 1.00 | 75.40 | | O |
| ANISOU | 1581 | O | THR | B | 34 | 10020 | 6281 | 12350 | −2640 | 525 | −2874 | O |
| ATOM | 1582 | CB | THR | B | 34 | 1.193 | 15.565 | −10.689 | 1.00 | 71.87 | | C |
| ANISOU | 1582 | CB | THR | B | 34 | 10340 | 6127 | 10841 | −2834 | 220 | −1839 | C |
| ATOM | 1583 | OG1 | THR | B | 34 | 0.699 | 15.436 | −9.352 | 1.00 | 77.51 | | O |
| ANISOU | 1583 | OG1 | THR | B | 34 | 12593 | 6759 | 10097 | −2385 | −298 | −2112 | O |
| ATOM | 1584 | CG2 | THR | B | 34 | 0.974 | 17.027 | −11.076 | 1.00 | 73.50 | | C |
| ANISOU | 1584 | CG2 | THR | B | 34 | 10481 | 6003 | 11443 | −2612 | 951 | −1969 | C |
| ATOM | 1585 | N | THR | B | 39 | 3.869 | 8.928 | −1.837 | 1.00 | 55.70 | | N |
| ANISOU | 1585 | N | THR | B | 39 | 7565 | 4557 | 9040 | 378 | −2654 | 2814 | N |
| ATOM | 1586 | CA | THR | B | 39 | 4.173 | 8.523 | −0.461 | 1.00 | 41.16 | | C |
| ANISOU | 1586 | CA | THR | B | 39 | 3742 | 4656 | 7240 | 891 | 64 | 1364 | C |
| ATOM | 1587 | C | THR | B | 39 | 5.628 | 8.121 | −0.345 | 1.00 | 42.80 | | C |
| ANISOU | 1587 | C | THR | B | 39 | 3887 | 5672 | 6705 | 1244 | −304 | 397 | C |
| ATOM | 1588 | O | THR | B | 39 | 6.171 | 7.406 | −1.191 | 1.00 | 49.80 | | O |
| ANISOU | 1588 | O | THR | B | 39 | 3963 | 7014 | 7944 | 1710 | −1199 | −1329 | O |
| ATOM | 1589 | CB | THR | B | 39 | 3.269 | 7.366 | 0.024 | 1.00 | 51.93 | | C |
| ANISOU | 1589 | CB | THR | B | 39 | 5388 | 6377 | 7965 | −925 | 108 | 1290 | C |
| ATOM | 1590 | OG1 | THR | B | 39 | 3.703 | 6.851 | 1.292 | 1.00 | 52.59 | | O |
| ANISOU | 1590 | OG1 | THR | B | 39 | 5812 | 7722 | 6448 | −1403 | 1796 | 1359 | O |
| ATOM | 1591 | CG2 | THR | B | 39 | 3.339 | 6.198 | −0.956 | 1.00 | 61.00 | | C |
| ANISOU | 1591 | CG2 | THR | B | 39 | 10484 | 5184 | 7510 | −1022 | −2364 | 2086 | C |
| ATOM | 1592 | N | ILE | B | 40 | 6.319 | 8.575 | 0.712 | 1.00 | 37.60 | | N |
| ANISOU | 1592 | N | ILE | B | 40 | 4275 | 4425 | 5584 | 1358 | 10 | 1251 | N |
| ATOM | 1593 | CA | ILE | B | 40 | 7.728 | 8.155 | 0.660 | 1.00 | 30.55 | | C |
| ANISOU | 1593 | CA | ILE | B | 40 | 3685 | 3387 | 4536 | 191 | 430 | 1091 | C |
| ATOM | 1594 | C | ILE | B | 40 | 7.891 | 6.936 | 1.557 | 1.00 | 23.64 | | C |
| ANISOU | 1594 | C | ILE | B | 40 | 2410 | 3108 | 3463 | 117 | 398 | 487 | C |
| ATOM | 1595 | O | ILE | B | 40 | 8.911 | 6.260 | 1.476 | 1.00 | 28.33 | | O |
| ANISOU | 1595 | O | ILE | B | 40 | 2927 | 3469 | 4369 | 631 | 134 | −32 | O |
| ATOM | 1596 | CB | ILE | B | 40 | 8.631 | 9.346 | 0.946 | 1.00 | 39.21 | | C |
| ANISOU | 1596 | CB | ILE | B | 40 | 5769 | 3488 | 5641 | −653 | 136 | 1232 | C |
| ATOM | 1597 | CG1 | ILE | B | 40 | 8.572 | 9.893 | 2.357 | 1.00 | 45.38 | | C |
| ANISOU | 1597 | CG1 | ILE | B | 40 | 8237 | 2760 | 6243 | 399 | −425 | 422 | C |
| ATOM | 1598 | CG2 | ILE | B | 40 | 8.342 | 10.484 | −0.038 | 1.00 | 46.98 | | C |
| ANISOU | 1598 | CG2 | ILE | B | 40 | 8136 | 3078 | 6637 | 150 | 395 | 1374 | C |
| ATOM | 1599 | CD1 | ILE | B | 40 | 8.084 | 11.278 | 2.648 | 1.00 | 50.75 | | C |
| ANISOU | 1599 | CD1 | ILE | B | 40 | 9321 | 2949 | 7013 | 1879 | −734 | 1395 | C |
| ATOM | 1600 | N | GLY | B | 41 | 6.918 | 6.547 | 2.371 | 1.00 | 23.99 | | N |
| ANISOU | 1600 | N | GLY | B | 41 | 2403 | 2837 | 3874 | −1325 | 70 | −204 | N |
| ATOM | 1601 | CA | GLY | B | 41 | 6.996 | 5.282 | 3.082 | 1.00 | 20.88 | | C |
| ANISOU | 1601 | CA | GLY | B | 41 | 1746 | 2838 | 3352 | −1167 | 196 | −399 | C |
| ATOM | 1602 | C | GLY | B | 41 | 5.967 | 4.279 | 2.695 | 1.00 | 14.33 | | C |
| ANISOU | 1602 | C | GLY | B | 41 | 1459 | 2221 | 1766 | −548 | 201 | −529 | C |
| ATOM | 1603 | O | GLY | B | 41 | 5.348 | 4.421 | 1.640 | 1.00 | 20.20 | | O |
| ANISOU | 1603 | O | GLY | B | 41 | 3600 | 2218 | 1859 | −794 | −426 | −87 | O |
| ATOM | 1604 | N | VAL | B | 42 | 5.793 | 3.283 | 3.578 | 1.00 | 12.30 | | N |
| ANISOU | 1604 | N | VAL | B | 42 | 1166 | 2024 | 1484 | −352 | −226 | −618 | N |
| ATOM | 1605 | CA | VAL | B | 42 | 4.769 | 2.282 | 3.298 | 1.00 | 10.97 | | C |
| ANISOU | 1605 | CA | VAL | B | 42 | 945 | 2003 | 1221 | −273 | 31 | −591 | C |
| ATOM | 1606 | C | VAL | B | 42 | 3.382 | 2.884 | 3.471 | 1.00 | 12.03 | | C |
| ANISOU | 1606 | C | VAL | B | 42 | 1049 | 2017 | 1505 | −148 | −236 | −889 | C |
| ATOM | 1607 | O | VAL | B | 42 | 3.155 | 3.625 | 4.436 | 1.00 | 12.00 | | O |
| ANISOU | 1607 | O | VAL | B | 42 | 1026 | 1933 | 1601 | −272 | −153 | −912 | O |
| ATOM | 1608 | CB | VAL | B | 42 | 4.968 | 1.101 | 4.253 | 1.00 | 11.92 | | C |
| ANISOU | 1608 | CB | VAL | B | 42 | 919 | 2203 | 1406 | −218 | 158 | −373 | C |
| ATOM | 1609 | CG1 | VAL | B | 42 | 3.848 | 0.090 | 4.069 | 1.00 | 14.62 | | C |
| ANISOU | 1609 | CG1 | VAL | B | 42 | 1446 | 2082 | 2027 | −471 | 72 | −517 | C |
| ATOM | 1610 | CG2 | VAL | B | 42 | 6.327 | 0.460 | 4.091 | 1.00 | 12.54 | | C |
| ANISOU | 1610 | CG2 | VAL | B | 42 | 1263 | 2244 | 1258 | 89 | 224 | −595 | C |
| ATOM | 1611 | N | GLU | B | 43 | 2.468 | 2.555 | 2.574 | 1.00 | 11.97 | | N |
| ANISOU | 1611 | N | GLU | B | 43 | 719 | 2357 | 1471 | −541 | 86 | −907 | N |
| ATOM | 1612 | CA | GLU | B | 43 | 1.103 | 3.040 | 2.626 | 1.00 | 11.96 | | C |
| ANISOU | 1612 | CA | GLU | B | 43 | 788 | 2027 | 1729 | −459 | −3 | −658 | C |
| ATOM | 1613 | C | GLU | B | 43 | 0.153 | 1.905 | 2.270 | 1.00 | 8.94 | | C |
| ANISOU | 1613 | C | GLU | B | 43 | 606 | 1451 | 1341 | −61 | −137 | −587 | C |
| ATOM | 1614 | O | GLU | B | 43 | 0.460 | 1.069 | 1.432 | 1.00 | 12.71 | | O |
| ANISOU | 1614 | O | GLU | B | 43 | 1116 | 2206 | 1508 | −192 | 353 | −1014 | O |
| ATOM | 1615 | CB | GLU | B | 43 | 0.878 | 4.217 | 1.650 | 1.00 | 17.42 | | C |
| ANISOU | 1615 | CB | GLU | B | 43 | 1440 | 1927 | 3250 | −581 | 285 | −22 | C |
| ATOM | 1616 | CG | GLU | B | 43 | −0.516 | 4.833 | 1.818 | 1.00 | 21.87 | | C |
| ANISOU | 1616 | CG | GLU | B | 43 | 2026 | 2276 | 4006 | 197 | 44 | −65 | C |
| ATOM | 1617 | CD | GLU | B | 43 | −0.742 | 5.926 | 0.763 | 1.00 | 30.32 | | C |
| ANISOU | 1617 | CD | GLU | B | 43 | 3176 | 3511 | 4831 | 707 | 281 | 959 | C |
| ATOM | 1618 | OE1 | GLU | B | 43 | 0.185 | 6.153 | −0.052 | 1.00 | 39.88 | | O |
| ANISOU | 1618 | OE1 | GLU | B | 43 | 6837 | 4601 | 3717 | 1584 | 2373 | 350 | O |
| ATOM | 1619 | OE2 | GLU | B | 43 | −1.818 | 6.561 | 0.744 | 1.00 | 40.65 | | O |
| ANISOU | 1619 | OE2 | GLU | B | 43 | 3285 | 4674 | 7486 | 1031 | −884 | 1724 | O |
| ATOM | 1620 | N | PHE | B | 44 | −0.987 | 1.873 | 2.904 | 1.00 | 8.03 | | N |
| ANISOU | 1620 | N | PHE | B | 44 | 562 | 1433 | 1056 | −28 | −192 | −539 | N |
| ATOM | 1621 | CA | PHE | B | 44 | −2.031 | 0.935 | 2.451 | 1.00 | 9.86 | | C |
| ANISOU | 1621 | CA | PHE | B | 44 | 637 | 1417 | 1694 | −182 | −253 | −232 | C |
| ATOM | 1622 | C | PHE | B | 44 | −3.370 | 1.618 | 2.526 | 1.00 | 10.10 | | C |
| ANISOU | 1622 | C | PHE | B | 44 | 535 | 1908 | 1393 | −198 | −133 | −559 | C |
| ATOM | 1623 | O | PHE | B | 44 | −3.660 | 2.476 | 3.375 | 1.00 | 11.78 | | O |
| ANISOU | 1623 | O | PHE | B | 44 | 907 | 1815 | 1755 | −96 | −178 | −703 | O |
| ATOM | 1624 | CB | PHE | B | 44 | −2.009 | −0.371 | 3.241 | 1.00 | 11.54 | | C |
| ANISOU | 1624 | CB | PHE | B | 44 | 1418 | 1543 | 1424 | −240 | 40 | −232 | C |
| ATOM | 1625 | CG | PHE | B | 44 | −2.049 | −0.271 | 4.746 | 1.00 | 13.35 | | C |
| ANISOU | 1625 | CG | PHE | B | 44 | 2111 | 1467 | 1497 | −301 | 149 | −469 | C |
| ATOM | 1626 | CD1 | PHE | B | 44 | −0.863 | −0.007 | 5.447 | 1.00 | 15.07 | | C |
| ANISOU | 1626 | CD1 | PHE | B | 44 | 2447 | 1732 | 1545 | 417 | −499 | −199 | C |
| ATOM | 1627 | CD2 | PHE | B | 44 | −3.208 | −0.434 | 5.434 | 1.00 | 15.79 | | C |
| ANISOU | 1627 | CD2 | PHE | B | 44 | 2520 | 1849 | 1631 | 29 | 644 | −771 | C |
| ATOM | 1628 | CE1 | PHE | B | 44 | −0.879 | 0.114 | 6.796 | 1.00 | 14.40 | | C |
| ANISOU | 1628 | CE1 | PHE | B | 44 | 1554 | 2244 | 1674 | 551 | 9 | −629 | C |
| ATOM | 1629 | CE2 | PHE | B | 44 | −3.238 | −0.335 | 6.836 | 1.00 | 13.13 | | C |
| ANISOU | 1629 | CE2 | PHE | B | 44 | 1884 | 1727 | 1379 | −206 | −117 | 207 | C |
| ATOM | 1630 | CZ | PHE | B | 44 | −2.065 | −0.110 | 7.480 | 1.00 | 16.08 | | C |
| ANISOU | 1630 | CZ | PHE | B | 44 | 1327 | 2280 | 2502 | 369 | 65 | −666 | C |
| ATOM | 1631 | N | LEU | B | 45 | −4.252 | 1.246 | 1.590 | 1.00 | 11.44 | | N |
| ANISOU | 1631 | N | LEU | B | 45 | 792 | 1748 | 1807 | 111 | −539 | −638 | N |
| ATOM | 1632 | CA | LEU | B | 45 | −5.596 | 1.744 | 1.373 | 1.00 | 11.62 | | C |
| ANISOU | 1632 | CA | LEU | B | 45 | 584 | 2061 | 1770 | −6 | −316 | −485 | C |
| ATOM | 1633 | C | LEU | B | 45 | −6.607 | 0.604 | 1.366 | 1.00 | 11.53 | | C |
| ANISOU | 1633 | C | LEU | B | 45 | 714 | 2056 | 1610 | −71 | 307 | −808 | C |
| ATOM | 1634 | O | LEU | B | 45 | −6.247 | −0.539 | 1.068 | 1.00 | 13.76 | | O |
| ANISOU | 1634 | O | LEU | B | 45 | 928 | 2024 | 2277 | 96 | 129 | −867 | O |
| ATOM | 1635 | CB | LEU | B | 45 | −5.714 | 2.482 | 0.017 | 1.00 | 15.69 | | C |
| ANISOU | 1635 | CB | LEU | B | 45 | 1008 | 2396 | 2559 | 9 | −620 | 287 | C |
| ATOM | 1636 | CG | LEU | B | 45 | −4.921 | 3.720 | −0.277 | 1.00 | 22.32 | | C |
| ANISOU | 1636 | CG | LEU | B | 45 | 2063 | 3118 | 3297 | −704 | −1025 | 872 | C |
| ATOM | 1637 | CD1 | LEU | B | 45 | −5.231 | 4.163 | −1.683 | 1.00 | 29.42 | | C |
| ANISOU | 1637 | CD1 | LEU | B | 45 | 5687 | 1957 | 3534 | −1124 | −1970 | 804 | C |
| ATOM | 1638 | CD2 | LEU | B | 45 | −5.337 | 4.899 | 0.582 | 1.00 | 43.04 | | C |
| ANISOU | 1638 | CD2 | LEU | B | 45 | 7924 | 3647 | 4781 | −1375 | −3231 | −1584 | C |
| ATOM | 1639 | N | ASN | B | 46 | −7.859 | 0.818 | 1.679 | 1.00 | 11.26 | | N |
| ANISOU | 1639 | N | ASN | B | 46 | 724 | 1960 | 1596 | 22 | 124 | −675 | N |
| ATOM | 1640 | CA | ASN | B | 46 | −8.955 | −0.105 | 1.637 | 1.00 | 10.31 | | C |
| ANISOU | 1640 | CA | ASN | B | 46 | 501 | 1844 | 1572 | 189 | 58 | −634 | C |
| ATOM | 1641 | C | ASN | B | 46 | −9.992 | 0.285 | 0.599 | 1.00 | 10.24 | | C |
| ANISOU | 1641 | C | ASN | B | 46 | 854 | 1636 | 1401 | 336 | −25 | −883 | C |
| ATOM | 1642 | O | ASN | B | 46 | −10.197 | 1.456 | 0.286 | 1.00 | 12.01 | | O |
| ANISOU | 1642 | O | ASN | B | 46 | 1123 | 1595 | 1844 | 101 | −209 | −571 | O |
| ATOM | 1643 | CB | ASN | B | 46 | −9.637 | −0.149 | 3.005 | 1.00 | 11.50 | | C |
| ANISOU | 1643 | CB | ASN | B | 46 | 686 | 2168 | 1517 | 66 | 92 | −581 | C |
| ATOM | 1644 | CG | ASN | B | 46 | −8.665 | −0.509 | 4.102 | 1.00 | 14.08 | | C |
| ANISOU | 1644 | CG | ASN | B | 46 | 1083 | 2717 | 1550 | 378 | −196 | −871 | C |
| ATOM | 1645 | OD1 | ASN | B | 46 | −8.140 | −1.619 | 4.171 | 1.00 | 13.90 | | O |
| ANISOU | 1645 | OD1 | ASN | B | 46 | 1120 | 2593 | 1570 | 123 | −53 | −362 | O |
| ATOM | 1646 | ND2 | ASN | B | 46 | −8.424 | 0.424 | 5.014 | 1.00 | 16.29 | | N |
| ANISOU | 1646 | ND2 | ASN | B | 46 | 1776 | 2823 | 1591 | −343 | −48 | −799 | N |
| ATOM | 1647 | N | LYS | B | 47 | −10.624 | −0.771 | 0.079 | 1.00 | 12.22 | | N |
| ANISOU | 1647 | N | LYS | B | 47 | 1046 | 1593 | 2006 | −220 | −374 | −174 | N |
| ATOM | 1648 | CA | LYS | B | 47 | −11.659 | −0.621 | −0.946 | 1.00 | 12.09 | | C |
| ANISOU | 1648 | CA | LYS | B | 47 | 817 | 2029 | 1749 | −133 | −162 | −504 | C |
| ATOM | 1649 | C | LYS | B | 47 | −12.678 | −1.739 | −0.761 | 1.00 | 10.94 | | C |
| ANISOU | 1649 | C | LYS | B | 47 | 696 | 1809 | 1651 | 1 | −94 | −503 | C |
| ATOM | 1650 | O | LYS | B | 47 | −12.321 | −2.908 | −0.719 | 1.00 | 12.08 | | O |
| ANISOU | 1650 | O | LYS | B | 47 | 793 | 1870 | 1928 | 105 | −250 | −504 | O |
| ATOM | 1651 | CB | LYS | B | 47 | −11.107 | −0.677 | −2.377 | 1.00 | 16.90 | | C |
| ANISOU | 1651 | CB | LYS | B | 47 | 1240 | 3375 | 1805 | −757 | 65 | −446 | C |
| ATOM | 1652 | CG | LYS | B | 47 | −12.217 | −0.378 | −3.385 | 1.00 | 23.77 | | C |
| ANISOU | 1652 | CG | LYS | B | 47 | 2089 | 5021 | 1921 | −1666 | −777 | −484 | C |
| ATOM | 1653 | CD | LYS | B | 47 | −11.824 | 0.434 | −4.557 | 1.00 | 21.72 | | C |
| ANISOU | 1653 | CD | LYS | B | 47 | 2348 | 3547 | 2357 | −499 | −707 | −313 | C |
| ATOM | 1654 | CE | LYS | B | 47 | −12.921 | 0.702 | −5.561 | 1.00 | 19.63 | | C |
| ANISOU | 1654 | CE | LYS | B | 47 | 1488 | 3746 | 2225 | 27 | −142 | −717 | C |
| ATOM | 1655 | NZ | LYS | B | 47 | −13.746 | 1.906 | −5.250 | 1.00 | 32.74 | | N |
| ANISOU | 1655 | NZ | LYS | B | 47 | 4871 | 5041 | 2528 | 2206 | 280 | −284 | N |
| ATOM | 1656 | N | ASP | B | 48 | −13.939 | −1.377 | −0.722 | 1.00 | 11.52 | | N |
| ANISOU | 1656 | N | ASP | B | 48 | 777 | 1679 | 1921 | 28 | −262 | −482 | N |
| ATOM | 1657 | CA | ASP | B | 48 | −15.051 | −2.309 | −0.720 | 1.00 | 12.16 | | C |
| ANISOU | 1657 | CA | ASP | B | 48 | 625 | 2057 | 1939 | −22 | −315 | −526 | C |
| ATOM | 1658 | C | ASP | B | 48 | −15.379 | −2.772 | −2.137 | 1.00 | 12.07 | | C |
| ANISOU | 1658 | C | ASP | B | 48 | 928 | 1829 | 1828 | −151 | −268 | −332 | C |
| ATOM | 1659 | O | ASP | B | 48 | −15.414 | −1.931 | −3.055 | 1.00 | 14.06 | | O |
| ANISOU | 1659 | O | ASP | B | 48 | 1424 | 1886 | 2033 | −406 | −399 | −197 | O |
| ATOM | 1660 | CB | ASP | B | 48 | −16.267 | −1.647 | −0.115 | 1.00 | 15.28 | | C |
| ANISOU | 1660 | CB | ASP | B | 48 | 809 | 2408 | 2587 | 62 | −54 | −838 | C |
| ATOM | 1661 | CG | ASP | B | 48 | −16.319 | −1.624 | 1.389 | 1.00 | 17.21 | | C |
| ANISOU | 1661 | CG | ASP | B | 48 | 1433 | 2495 | 2611 | 200 | 150 | −1015 | C |
| ATOM | 1662 | OD1 | ASP | B | 48 | −15.475 | −2.267 | 2.027 | 1.00 | 19.27 | | O |
| ANISOU | 1662 | OD1 | ASP | B | 48 | 1649 | 3296 | 2376 | 390 | 109 | −790 | O |
| ATOM | 1663 | OD2 | ASP | B | 48 | −17.280 | −0.991 | 1.869 | 1.00 | 21.28 | | O |
| ANISOU | 1663 | OD2 | ASP | B | 48 | 2131 | 2853 | 3100 | 678 | 569 | −828 | O |
| ATOM | 1664 | N | LEU | B | 49 | −15.589 | −4.087 | −2.262 | 1.00 | 12.11 | | N |
| ANISOU | 1664 | N | LEU | B | 49 | 1148 | 1787 | 1667 | −81 | −398 | −255 | N |
| ATOM | 1665 | CA | LEU | B | 49 | −16.070 | −4.616 | −3.543 | 1.00 | 12.92 | | C |
| ANISOU | 1665 | CA | LEU | B | 49 | 1453 | 1990 | 1466 | −283 | 49 | −396 | C |
| ATOM | 1666 | C | LEU | B | 49 | −17.012 | −5.761 | −3.230 | 1.00 | 11.04 | | C |
| ANISOU | 1666 | C | LEU | B | 49 | 1216 | 1784 | 1194 | −84 | −232 | −322 | C |
| ATOM | 1667 | O | LEU | B | 49 | −17.378 | −5.998 | −2.084 | 1.00 | 11.51 | | O |
| ANISOU | 1667 | O | LEU | B | 49 | 764 | 2402 | 1206 | −224 | −78 | −502 | O |
| ATOM | 1668 | CB | LEU | B | 49 | −14.961 | −4.979 | −4.510 | 1.00 | 16.96 | | C |
| ANISOU | 1668 | CB | LEU | B | 49 | 1038 | 3054 | 2352 | −438 | 123 | −1094 | C |
| ATOM | 1669 | CG | LEU | B | 49 | −13.950 | −6.021 | −4.213 | 1.00 | 16.30 | | C |
| ANISOU | 1669 | CG | LEU | B | 49 | 1361 | 3158 | 1673 | −237 | 202 | −934 | C |
| ATOM | 1670 | CD1 | LEU | B | 49 | −13.247 | −6.467 | −5.498 | 1.00 | 24.92 | | C |
| ANISOU | 1670 | CD1 | LEU | B | 49 | 3342 | 4023 | 2105 | 782 | 200 | −1902 | C |
| ATOM | 1671 | CD2 | LEU | B | 49 | −12.886 | −5.506 | −3.259 | 1.00 | 19.67 | | C |
| ANISOU | 1671 | CD2 | LEU | B | 49 | 1662 | 3525 | 2285 | 431 | −279 | −1647 | C |
| ATOM | 1672 | N | GLU | B | 50 | −17.425 | −6.478 | −4.248 | 1.00 | 12.51 | | N |
| ANISOU | 1672 | N | GLU | B | 50 | 1728 | 1857 | 1169 | −307 | −58 | −348 | N |
| ATOM | 1673 | CA | GLU | B | 50 | −18.315 | −7.587 | −4.053 | 1.00 | 13.49 | | C |
| ANISOU | 1673 | CA | GLU | B | 50 | 1396 | 1962 | 1769 | −359 | 45 | −478 | C |
| ATOM | 1674 | C | GLU | B | 50 | −17.944 | −8.676 | −5.046 | 1.00 | 12.17 | | C |
| ANISOU | 1674 | C | GLU | B | 50 | 1167 | 2108 | 1350 | −332 | −414 | −485 | C |
| ATOM | 1675 | O | GLU | B | 50 | −17.543 | −8.327 | −6.170 | 1.00 | 16.04 | | O |
| ANISOU | 1675 | O | GLU | B | 50 | 2172 | 2227 | 1697 | −511 | 183 | −462 | O |
| ATOM | 1676 | CB | GLU | B | 50 | −19.747 | −7.044 | −4.264 | 1.00 | 15.96 | | C |
| ANISOU | 1676 | CB | GLU | B | 50 | 1576 | 2805 | 1684 | 20 | 160 | −352 | C |
| ATOM | 1677 | CG | GLU | B | 50 | −20.819 | −8.003 | −3.887 | 1.00 | 16.43 | | C |
| ANISOU | 1677 | CG | GLU | B | 50 | 1353 | 3142 | 1748 | −138 | −168 | −520 | C |
| ATOM | 1678 | CD | GLU | B | 50 | −22.200 | −7.376 | −3.804 | 1.00 | 16.70 | | C |
| ANISOU | 1678 | CD | GLU | B | 50 | 1318 | 2087 | 2941 | −241 | −801 | 112 | C |
| ATOM | 1679 | OE1 | GLU | B | 50 | −22.271 | −6.148 | −3.722 | 1.00 | 21.39 | | O |
| ANISOU | 1679 | OE1 | GLU | B | 50 | 2525 | 2086 | 3515 | −276 | −269 | −179 | O |
| ATOM | 1680 | OE2 | GLU | B | 50 | −23.148 | −8.177 | −3.838 | 1.00 | 22.18 | | O |
| ANISOU | 1680 | OE2 | GLU | B | 50 | 1456 | 2534 | 4435 | −590 | −240 | 301 | O |
| ATOM | 1681 | N | VAL | B | 51 | −18.020 | −9.934 | −4.625 | 1.00 | 11.42 | | N |
| ANISOU | 1681 | N | VAL | B | 51 | 802 | 2099 | 1437 | −36 | 65 | −516 | N |
| ATOM | 1682 | CA | VAL | B | 51 | −17.674 | −11.058 | −5.480 | 1.00 | 12.54 | | C |
| ANISOU | 1682 | CA | VAL | B | 51 | 1261 | 2223 | 1281 | 30 | 62 | −558 | C |
| ATOM | 1683 | C | VAL | B | 51 | −18.743 | −12.132 | −5.316 | 1.00 | 12.90 | | C |
| ANISOU | 1683 | C | VAL | B | 51 | 1198 | 2205 | 1497 | 25 | 269 | −726 | C |
| ATOM | 1684 | O | VAL | B | 51 | −18.994 | −12.526 | −4.190 | 1.00 | 12.22 | | O |
| ANISOU | 1684 | O | VAL | B | 51 | 1055 | 2099 | 1488 | 149 | 93 | −521 | O |
| ATOM | 1685 | CB | VAL | B | 51 | −16.229 | −11.570 | −5.231 | 1.00 | 14.71 | | C |
| ANISOU | 1685 | CB | VAL | B | 51 | 1085 | 2552 | 1951 | 190 | 306 | −673 | C |
| ATOM | 1686 | CG1 | VAL | B | 51 | −15.925 | −12.780 | −6.100 | 1.00 | 18.82 | | C |
| ANISOU | 1686 | CG1 | VAL | B | 51 | 1846 | 3226 | 2080 | 643 | 379 | −944 | C |
| ATOM | 1687 | CG2 | VAL | B | 51 | −15.224 | −10.473 | −5.510 | 1.00 | 25.64 | | C |
| ANISOU | 1687 | CG2 | VAL | B | 51 | 2032 | 4738 | 2973 | −1594 | 485 | −1309 | C |
| ATOM | 1688 | N | ASP | B | 52 | −19.342 | −12.579 | −6.430 | 1.00 | 12.85 | | N |
| ANISOU | 1688 | N | ASP | B | 52 | 1090 | 2295 | 1499 | −128 | 82 | −350 | N |
| ATOM | 1689 | CA | ASP | B | 52 | −20.398 | −13.593 | −6.331 | 1.00 | 14.53 | | C |
| ANISOU | 1689 | CA | ASP | B | 52 | 1176 | 2348 | 1996 | −192 | 184 | −573 | C |
| ATOM | 1690 | C | ASP | B | 52 | −21.499 | −13.246 | −5.336 | 1.00 | 13.90 | | C |
| ANISOU | 1690 | C | ASP | B | 52 | 1192 | 2168 | 1921 | −165 | 205 | −434 | C |
| ATOM | 1691 | O | ASP | B | 52 | −22.059 | −14.104 | −4.660 | 1.00 | 13.57 | | O |
| ANISOU | 1691 | O | ASP | B | 52 | 1409 | 1871 | 1876 | −326 | 237 | −787 | O |
| ATOM | 1692 | CB | ASP | B | 52 | −19.756 | −14.926 | −5.985 | 1.00 | 15.99 | | C |
| ANISOU | 1692 | CB | ASP | B | 52 | 1369 | 2233 | 2472 | −177 | 646 | −496 | C |
| ATOM | 1693 | CG | ASP | B | 52 | −18.730 | −15.414 | −6.993 | 1.00 | 16.28 | | C |
| ANISOU | 1693 | CG | ASP | B | 52 | 1051 | 2817 | 2316 | −22 | 480 | −342 | C |
| ATOM | 1694 | OD1 | ASP | B | 52 | −18.847 | −15.081 | −8.188 | 1.00 | 17.46 | | O |
| ANISOU | 1694 | OD1 | ASP | B | 52 | 2008 | 2327 | 2297 | −353 | 593 | −348 | O |
| ATOM | 1695 | OD2 | ASP | B | 52 | −17.824 | −16.167 | −6.569 | 1.00 | 23.31 | | O |
| ANISOU | 1695 | OD2 | ASP | B | 52 | 2245 | 3428 | 3182 | 1023 | 616 | −116 | O |
| ATOM | 1696 | N | GLY | B | 53 | −21.827 | −11.951 | −5.223 | 1.00 | 11.67 | | N |
| ANISOU | 1696 | N | GLY | B | 53 | 802 | 2080 | 1551 | −334 | −92 | −475 | N |
| ATOM | 1697 | CA | GLY | B | 53 | −22.869 | −11.539 | −4.330 | 1.00 | 11.47 | | C |
| ANISOU | 1697 | CA | GLY | B | 53 | 756 | 2108 | 1494 | 44 | −235 | −181 | C |
| ATOM | 1698 | C | GLY | B | 53 | −22.452 | −11.348 | −2.901 | 1.00 | 11.82 | | C |
| ANISOU | 1698 | C | GLY | B | 53 | 789 | 2165 | 1539 | −67 | −142 | −408 | C |
| ATOM | 1699 | O | GLY | B | 53 | −23.286 | −11.003 | −2.066 | 1.00 | 13.52 | | O |
| ANISOU | 1699 | O | GLY | B | 53 | 996 | 2461 | 1679 | −141 | −2 | −559 | O |
| ATOM | 1700 | N | HIS | B | 54 | −21.189 | −11.568 | −2.566 | 1.00 | 10.66 | | N |
| ANISOU | 1700 | N | HIS | B | 54 | 935 | 1657 | 1460 | −87 | −397 | −380 | N |
| ATOM | 1701 | CA | HIS | B | 54 | −20.704 | −11.413 | −1.201 | 1.00 | 10.78 | | C |
| ANISOU | 1701 | CA | HIS | B | 54 | 1098 | 1798 | 1198 | −22 | −117 | −221 | C |
| ATOM | 1702 | C | HIS | B | 54 | −19.881 | −10.146 | −1.028 | 1.00 | 11.02 | | C |
| ANISOU | 1702 | C | HIS | B | 54 | 988 | 2361 | 837 | −437 | −147 | −333 | C |
| ATOM | 1703 | O | HIS | B | 54 | −19.009 | −9.864 | −1.863 | 1.00 | 12.81 | | O |
| ANISOU | 1703 | O | HIS | B | 54 | 1037 | 2634 | 1197 | −439 | 50 | −618 | O |
| ATOM | 1704 | CB | HIS | B | 54 | −19.738 | −12.542 | −0.866 | 1.00 | 14.73 | | C |
| ANISOU | 1704 | CB | HIS | B | 54 | 1202 | 2462 | 1934 | 223 | −469 | 88 | C |
| ATOM | 1705 | CG | HIS | B | 54 | −20.320 | −13.906 | −0.829 | 1.00 | 15.99 | | C |
| ANISOU | 1705 | CG | HIS | B | 54 | 2336 | 1929 | 1811 | 527 | 125 | −113 | C |
| ATOM | 1706 | ND1 | HIS | B | 54 | −20.196 | −14.676 | 0.313 | 1.00 | 21.83 | | N |
| ANISOU | 1706 | ND1 | HIS | B | 54 | 3023 | 2908 | 2364 | −115 | −385 | 560 | N |
| ATOM | 1707 | CD2 | HIS | B | 54 | −20.999 | −14.633 | −1.742 | 1.00 | 20.49 | | C |
| ANISOU | 1707 | CD2 | HIS | B | 54 | 3545 | 1966 | 2275 | −109 | −411 | 130 | C |
| ATOM | 1708 | CE1 | HIS | B | 54 | −20.790 | −15.838 | 0.079 | 1.00 | 22.61 | | C |
| ANISOU | 1708 | CE1 | HIS | B | 54 | 3241 | 3027 | 2323 | −483 | 291 | 679 | C |
| ATOM | 1709 | NE2 | HIS | B | 54 | −21.283 | −15.845 | −1.157 | 1.00 | 20.12 | | N |
| ANISOU | 1709 | NE2 | HIS | B | 54 | 2884 | 2176 | 2587 | −7 | 20 | 291 | N |
| ATOM | 1710 | N | PHE | B | 55 | −20.130 | −9.427 | 0.048 | 1.00 | 11.46 | | N |
| ANISOU | 1710 | N | PHE | B | 55 | 871 | 2324 | 1160 | −416 | −24 | −436 | N |
| ATOM | 1711 | CA | PHE | B | 55 | −19.410 | −8.230 | 0.389 | 1.00 | 11.13 | | C |
| ANISOU | 1711 | CA | PHE | B | 55 | 664 | 2067 | 1496 | −200 | −235 | −389 | C |
| ATOM | 1712 | C | PHE | B | 55 | −17.988 | −8.585 | 0.855 | 1.00 | 12.33 | | C |
| ANISOU | 1712 | C | PHE | B | 55 | 681 | 2242 | 1760 | −243 | −174 | 153 | C |
| ATOM | 1713 | O | PHE | B | 55 | −17.773 | −9.447 | 1.718 | 1.00 | 15.02 | | O |
| ANISOU | 1713 | O | PHE | B | 55 | 1078 | 2430 | 2200 | −216 | −274 | 446 | O |
| ATOM | 1714 | CB | PHE | B | 55 | −20.077 | −7.454 | 1.505 | 1.00 | 11.78 | | C |
| ANISOU | 1714 | CB | PHE | B | 55 | 998 | 2200 | 1277 | −131 | −318 | −368 | C |
| ATOM | 1715 | CG | PHE | B | 55 | −21.515 | −7.016 | 1.241 | 1.00 | 10.37 | | C |
| ANISOU | 1715 | CG | PHE | B | 55 | 1077 | 1711 | 1152 | 24 | −264 | −535 | C |
| ATOM | 1716 | CD1 | PHE | B | 55 | −21.983 | −6.822 | −0.041 | 1.00 | 13.42 | | C |
| ANISOU | 1716 | CD1 | PHE | B | 55 | 920 | 2896 | 1283 | −226 | −305 | −148 | C |
| ATOM | 1717 | CD2 | PHE | B | 55 | −22.358 | −6.819 | 2.285 | 1.00 | 11.95 | | C |
| ANISOU | 1717 | CD2 | PHE | B | 55 | 1259 | 1902 | 1380 | −343 | 96 | −409 | C |
| ATOM | 1718 | CE1 | PHE | B | 55 | −23.280 | −6.403 | −0.249 | 1.00 | 11.94 | | C |
| ANISOU | 1718 | CE1 | PHE | B | 55 | 1129 | 2232 | 1174 | 93 | −273 | −491 | C |
| ATOM | 1719 | CE2 | PHE | B | 55 | −23.643 | −6.375 | 2.095 | 1.00 | 12.65 | | C |
| ANISOU | 1719 | CE2 | PHE | B | 55 | 1131 | 2327 | 1347 | −453 | 277 | −406 | C |
| ATOM | 1720 | CZ | PHE | B | 55 | −24.115 | −6.158 | 0.819 | 1.00 | 12.87 | | C |
| ANISOU | 1720 | CZ | PHE | B | 55 | 1507 | 1918 | 1465 | −10 | −29 | −685 | C |
| ATOM | 1721 | N | VAL | B | 56 | −17.014 | −7.931 | 0.256 | 1.00 | 10.68 | | N |
| ANISOU | 1721 | N | VAL | B | 56 | 689 | 1988 | 1382 | −304 | 44 | −427 | N |
| ATOM | 1722 | CA | VAL | B | 56 | −15.625 | −8.127 | 0.612 | 1.00 | 11.76 | | C |
| ANISOU | 1722 | CA | VAL | B | 56 | 751 | 2110 | 1606 | −203 | 10 | −135 | C |
| ATOM | 1723 | C | VAL | B | 56 | −14.907 | −6.774 | 0.616 | 1.00 | 11.88 | | C |
| ANISOU | 1723 | C | VAL | B | 56 | 529 | 2139 | 1846 | −180 | 24 | −317 | C |
| ATOM | 1724 | O | VAL | B | 56 | −15.360 | −5.780 | 0.053 | 1.00 | 11.74 | | O |
| ANISOU | 1724 | O | VAL | B | 56 | 793 | 1992 | 1678 | −158 | −207 | −523 | O |
| ATOM | 1725 | CB | VAL | B | 56 | −14.887 | −9.101 | −0.304 | 1.00 | 14.88 | | C |
| ANISOU | 1725 | CB | VAL | B | 56 | 871 | 2410 | 2371 | 177 | −395 | −682 | C |
| ATOM | 1726 | CG1 | VAL | B | 56 | −15.575 | −10.467 | −0.340 | 1.00 | 19.63 | | C |
| ANISOU | 1726 | CG1 | VAL | B | 56 | 1337 | 2680 | 3442 | −173 | −232 | −1242 | C |
| ATOM | 1727 | CG2 | VAL | B | 56 | −14.748 | −8.530 | −1.702 | 1.00 | 17.90 | | C |
| ANISOU | 1727 | CG2 | VAL | B | 56 | 1763 | 3388 | 1652 | −275 | −236 | −1390 | C |
| ATOM | 1728 | N | THR | B | 57 | −13.758 | −6.768 | 1.295 | 1.00 | 12.77 | | N |
| ANISOU | 1728 | N | THR | B | 57 | 786 | 2294 | 1773 | −223 | −168 | −241 | N |
| ATOM | 1729 | CA | THR | B | 57 | −12.921 | −5.567 | 1.347 | 1.00 | 12.02 | | C |
| ANISOU | 1729 | CA | THR | B | 57 | 665 | 2160 | 1742 | −47 | −64 | −851 | C |
| ATOM | 1730 | C | THR | B | 57 | −11.511 | −5.976 | 0.929 | 1.00 | 11.46 | | C |
| ANISOU | 1730 | C | THR | B | 57 | 768 | 1960 | 1625 | 17 | 28 | −699 | C |
| ATOM | 1731 | O | THR | B | 57 | −11.053 | −7.024 | 1.378 | 1.00 | 13.02 | | O |
| ANISOU | 1731 | O | THR | B | 57 | 817 | 2248 | 1883 | 130 | −61 | −354 | O |
| ATOM | 1732 | CB | THR | B | 57 | −12.920 | −4.934 | 2.751 | 1.00 | 12.86 | | C |
| ANISOU | 1732 | CB | THR | B | 57 | 1078 | 2352 | 1456 | 148 | 119 | −559 | C |
| ATOM | 1733 | OG1 | THR | B | 57 | −14.294 | −4.616 | 3.027 | 1.00 | 15.96 | | O |
| ANISOU | 1733 | OG1 | THR | B | 57 | 1086 | 3126 | 1854 | 118 | 249 | −833 | O |
| ATOM | 1734 | CG2 | THR | B | 57 | −12.081 | −3.684 | 2.854 | 1.00 | 16.64 | | C |
| ANISOU | 1734 | CG2 | THR | B | 57 | 1500 | 3131 | 1693 | −474 | −162 | −1233 | C |
| ATOM | 1735 | N | MET | B | 58 | −10.873 | −5.153 | 0.137 | 1.00 | 11.55 | | N |
| ANISOU | 1735 | N | MET | B | 58 | 466 | 1826 | 2096 | −62 | −203 | −561 | N |
| ATOM | 1736 | CA | MET | B | 58 | −9.489 | −5.282 | −0.281 | 1.00 | 13.51 | | C |
| ANISOU | 1736 | CA | MET | B | 58 | 529 | 2483 | 2120 | −296 | 15 | −1186 | C |
| ATOM | 1737 | C | MET | B | 58 | −8.637 | −4.262 | 0.456 | 1.00 | 10.91 | | C |
| ANISOU | 1737 | C | MET | B | 58 | 569 | 1879 | 1696 | −98 | 5 | −831 | C |
| ATOM | 1738 | O | MET | B | 58 | −9.049 | −3.096 | 0.548 | 1.00 | 13.35 | | O |
| ANISOU | 1738 | O | MET | B | 58 | 984 | 1788 | 2302 | 42 | −152 | −414 | O |
| ATOM | 1739 | CB | MET | B | 58 | −9.345 | −5.052 | −1.783 | 1.00 | 16.93 | | C |
| ANISOU | 1739 | CB | MET | B | 58 | 1436 | 2984 | 2014 | −353 | 98 | −1736 | C |
| ATOM | 1740 | CG | MET | B | 58 | −8.020 | −5.405 | −2.352 | 1.00 | 17.84 | | C |
| ANISOU | 1740 | CG | MET | B | 58 | 1570 | 3057 | 2152 | −538 | 465 | −1085 | C |
| ATOM | 1741 | SD | MET | B | 58 | −7.951 | −4.924 | −4.090 | 1.00 | 19.58 | | S |
| ANISOU | 1741 | SD | MET | B | 58 | 2262 | 2890 | 2288 | −709 | 370 | −684 | S |
| ATOM | 1742 | CE | MET | B | 58 | −9.513 | −5.463 | −4.717 | 1.00 | 38.63 | | C |
| ANISOU | 1742 | CE | MET | B | 58 | 4520 | 8398 | 1761 | −4428 | −103 | 2 | C |
| ATOM | 1743 | N | GLN | B | 59 | −7.494 | −4.688 | 0.966 | 1.00 | 11.72 | | N |
| ANISOU | 1743 | N | GLN | B | 59 | 591 | 1842 | 2021 | −97 | −150 | −763 | N |
| ATOM | 1744 | CA | GLN | B | 59 | −6.517 | −3.788 | 1.541 | 1.00 | 10.46 | | C |
| ANISOU | 1744 | CA | GLN | B | 59 | 720 | 1579 | 1677 | −92 | −134 | −687 | C |
| ATOM | 1745 | C | GLN | B | 59 | −5.252 | −3.880 | 0.689 | 1.00 | 10.21 | | C |
| ANISOU | 1745 | C | GLN | B | 59 | 669 | 1671 | 1540 | −47 | −175 | −466 | C |
| ATOM | 1746 | O | GLN | B | 59 | −4.621 | −4.924 | 0.603 | 1.00 | 10.72 | | O |
| ANISOU | 1746 | O | GLN | B | 59 | 801 | 1685 | 1586 | −3 | −57 | −311 | O |
| ATOM | 1747 | CB | GLN | B | 59 | −6.234 | −4.213 | 2.963 | 1.00 | 11.26 | | C |
| ANISOU | 1747 | CB | GLN | B | 59 | 748 | 1890 | 1641 | −28 | 29 | −694 | C |
| ATOM | 1748 | CG | GLN | B | 59 | −5.363 | −3.186 | 3.692 | 1.00 | 11.12 | | C |
| ANISOU | 1748 | CG | GLN | B | 59 | 902 | 1814 | 1510 | 71 | −277 | −483 | C |
| ATOM | 1749 | CD | GLN | B | 59 | −5.425 | −3.568 | 5.131 | 1.00 | 11.77 | | C |
| ANISOU | 1749 | CD | GLN | B | 59 | 1401 | 1587 | 1487 | −108 | −56 | −342 | C |
| ATOM | 1750 | OE1 | GLN | B | 59 | −6.278 | −3.104 | 5.943 | 1.00 | 16.67 | | O |
| ANISOU | 1750 | OE1 | GLN | B | 59 | 1503 | 2896 | 1934 | 274 | 193 | −669 | O |
| ATOM | 1751 | NE2 | GLN | B | 59 | −4.606 | −4.480 | 5.618 | 1.00 | 12.42 | | N |
| ANISOU | 1751 | NE2 | GLN | B | 59 | 1174 | 2117 | 1429 | −58 | −241 | −296 | N |
| ATOM | 1752 | N | ILE | B | 60 | −4.944 | −2.769 | 0.050 | 1.00 | 10.67 | | N |
| ANISOU | 1752 | N | ILE | B | 60 | 880 | 1613 | 1560 | 215 | 19 | −470 | N |
| ATOM | 1753 | CA | ILE | B | 60 | −3.881 | −2.677 | −0.908 | 1.00 | 10.19 | | C |
| ANISOU | 1753 | CA | ILE | B | 60 | 790 | 1810 | 1270 | −47 | −183 | −448 | C |
| ATOM | 1754 | C | ILE | B | 60 | −2.651 | −2.022 | −0.307 | 1.00 | 10.01 | | C |
| ANISOU | 1754 | C | ILE | B | 60 | 804 | 1624 | 1375 | 148 | −261 | −604 | C |
| ATOM | 1755 | O | ILE | B | 60 | −2.781 | −0.879 | 0.141 | 1.00 | 12.07 | | O |
| ANISOU | 1755 | O | ILE | B | 60 | 1154 | 1711 | 1723 | 356 | −476 | −793 | O |
| ATOM | 1756 | CB | ILE | B | 60 | −4.331 | −1.898 | −2.151 | 1.00 | 10.44 | | C |
| ANISOU | 1756 | CB | ILE | B | 60 | 962 | 1481 | 1524 | −260 | −402 | −381 | C |
| ATOM | 1757 | CG1 | ILE | B | 60 | −5.607 | −2.471 | −2.769 | 1.00 | 12.96 | | C |
| ANISOU | 1757 | CG1 | ILE | B | 60 | 1221 | 2037 | 1664 | −316 | −669 | −512 | C |
| ATOM | 1758 | CG2 | ILE | B | 60 | −3.200 | −1.851 | −3.169 | 1.00 | 11.33 | | C |
| ANISOU | 1758 | CG2 | ILE | B | 60 | 1339 | 1288 | 1677 | 19 | −151 | 13 | C |
| ATOM | 1759 | CD1 | ILE | B | 60 | −6.341 | −1.448 | −3.571 | 1.00 | 20.01 | | C |
| ANISOU | 1759 | CD1 | ILE | B | 60 | 2083 | 1807 | 3714 | −46 | −1942 | −680 | C |
| ATOM | 1760 | N | TRP | B | 61 | −1.540 | −2.741 | −0.314 | 1.00 | 8.92 | | N |
| ANISOU | 1760 | N | TRP | B | 61 | 652 | 1518 | 1220 | −6 | −127 | −790 | N |
| ATOM | 1761 | CA | TRP | B | 61 | −0.286 | −2.272 | 0.268 | 1.00 | 9.10 | | C |
| ANISOU | 1761 | CA | TRP | B | 61 | 776 | 1667 | 1014 | −159 | −193 | −620 | C |
| ATOM | 1762 | C | TRP | B | 61 | 0.709 | −1.882 | −0.777 | 1.00 | 9.88 | | C |
| ANISOU | 1762 | C | TRP | B | 61 | 824 | 1690 | 1241 | −188 | −64 | −539 | C |
| ATOM | 1763 | O | TRP | B | 61 | 0.950 | −2.649 | −1.723 | 1.00 | 11.99 | | O |
| ANISOU | 1763 | O | TRP | B | 61 | 1318 | 1902 | 1335 | −690 | 347 | −698 | O |
| ATOM | 1764 | CB | TRP | B | 61 | 0.334 | −3.394 | 1.116 | 1.00 | 11.02 | | C |
| ANISOU | 1764 | CB | TRP | B | 61 | 894 | 2014 | 1279 | 66 | −128 | −331 | C |
| ATOM | 1765 | CG | TRP | B | 61 | −0.376 | −3.613 | 2.398 | 1.00 | 9.52 | | C |
| ANISOU | 1765 | CG | TRP | B | 61 | 799 | 1588 | 1232 | −21 | −228 | −441 | C |
| ATOM | 1766 | CD1 | TRP | B | 61 | −1.633 | −4.106 | 2.489 | 1.00 | 10.18 | | C |
| ANISOU | 1766 | CD1 | TRP | B | 61 | 897 | 1785 | 1185 | −33 | −344 | −133 | C |
| ATOM | 1767 | CD2 | TRP | B | 61 | 0.117 | −3.330 | 3.714 | 1.00 | 8.70 | | C |
| ANISOU | 1767 | CD2 | TRP | B | 61 | 891 | 1226 | 1190 | 107 | −279 | −180 | C |
| ATOM | 1768 | NE1 | TRP | B | 61 | −1.832 | −4.113 | 3.797 | 1.00 | 11.62 | | N |
| ANISOU | 1768 | NE1 | TRP | B | 61 | 912 | 2151 | 1352 | −99 | −183 | −337 | N |
| ATOM | 1769 | CE2 | TRP | B | 61 | −0.890 | −3.682 | 4.642 | 1.00 | 9.59 | | C |
| ANISOU | 1769 | CE2 | TRP | B | 61 | 910 | 1558 | 1178 | 15 | −263 | −215 | C |
| ATOM | 1770 | CE3 | TRP | B | 61 | 1.308 | −2.820 | 4.193 | 1.00 | 9.92 | | C |
| ANISOU | 1770 | CE3 | TRP | B | 61 | 902 | 1781 | 1086 | −59 | −259 | −201 | C |
| ATOM | 1771 | CZ2 | TRP | B | 61 | −0.781 | −3.554 | 6.030 | 1.00 | 10.33 | | C |
| ANISOU | 1771 | CZ2 | TRP | B | 61 | 1024 | 1662 | 1237 | 89 | −56 | −443 | C |
| ATOM | 1772 | CZ3 | TRP | B | 61 | 1.423 | −2.696 | 5.567 | 1.00 | 10.86 | | C |
| ANISOU | 1772 | CZ3 | TRP | B | 61 | 797 | 2155 | 1173 | 253 | −163 | −514 | C |
| ATOM | 1773 | CH2 | TRP | B | 61 | 0.429 | −3.053 | 6.473 | 1.00 | 10.62 | | C |
| ANISOU | 1773 | CH2 | TRP | B | 61 | 939 | 1929 | 1166 | 199 | 103 | −1003 | C |
| ATOM | 1774 | N | ASP | B | 62 | 1.353 | −0.757 | −0.626 | 1.00 | 9.33 | | N |
| ANISOU | 1774 | N | ASP | B | 62 | 975 | 1587 | 984 | −113 | −123 | −472 | N |
| ATOM | 1775 | CA | ASP | B | 62 | 2.483 | −0.309 | −1.391 | 1.00 | 9.39 | | C |
| ANISOU | 1775 | CA | ASP | B | 62 | 1158 | 1334 | 1075 | −177 | 7 | −490 | C |
| ATOM | 1776 | C | ASP | B | 62 | 3.638 | −0.218 | −0.395 | 1.00 | 10.40 | | C |
| ANISOU | 1776 | C | ASP | B | 62 | 918 | 1706 | 1329 | −195 | 17 | −504 | C |
| ATOM | 1777 | O | ASP | B | 62 | 3.671 | 0.730 | 0.402 | 1.00 | 12.16 | | O |
| ANISOU | 1777 | O | ASP | B | 62 | 1183 | 1609 | 1829 | −111 | −481 | −623 | O |
| ATOM | 1778 | CB | ASP | B | 62 | 2.433 | 1.091 | −1.904 | 1.00 | 14.18 | | C |
| ANISOU | 1778 | CB | ASP | B | 62 | 2135 | 1768 | 1484 | −188 | −21 | 18 | C |
| ATOM | 1779 | CG | ASP | B | 62 | 3.367 | 1.576 | −2.951 | 1.00 | 12.20 | | C |
| ANISOU | 1779 | CG | ASP | B | 62 | 1654 | 1767 | 1213 | −11 | −260 | 160 | C |
| ATOM | 1780 | OD1 | ASP | B | 62 | 4.395 | 0.892 | −3.075 | 1.00 | 14.13 | | O |
| ANISOU | 1780 | OD1 | ASP | B | 62 | 1585 | 1449 | 2335 | −139 | −228 | 146 | O |
| ATOM | 1781 | OD2 | ASP | B | 62 | 3.089 | 2.638 | −3.500 | 1.00 | 17.01 | | O |
| ANISOU | 1781 | OD2 | ASP | B | 62 | 1571 | 1942 | 2950 | 39 | −52 | 803 | O |
| ATOM | 1782 | N | THR | B | 63 | 4.495 | −1.211 | −0.461 | 1.00 | 12.82 | | N |
| ANISOU | 1782 | N | THR | B | 63 | 1578 | 1808 | 1484 | 143 | −449 | −466 | N |
| ATOM | 1783 | CA | THR | B | 63 | 5.521 | −1.344 | 0.570 | 1.00 | 13.36 | | C |
| ANISOU | 1783 | CA | THR | B | 63 | 1391 | 2252 | 1435 | −124 | −373 | −39 | C |
| ATOM | 1784 | C | THR | B | 63 | 6.845 | −0.734 | 0.205 | 1.00 | 12.87 | | C |
| ANISOU | 1784 | C | THR | B | 63 | 1282 | 2280 | 1329 | 122 | −4 | −408 | C |
| ATOM | 1785 | O | THR | B | 63 | 7.762 | −0.910 | 1.018 | 1.00 | 16.35 | | O |
| ANISOU | 1785 | O | THR | B | 63 | 1356 | 2912 | 1945 | −55 | −383 | −239 | O |
| ATOM | 1786 | CB | THR | B | 63 | 5.752 | −2.838 | 0.882 | 1.00 | 13.05 | | C |
| ANISOU | 1786 | CB | THR | B | 63 | 1032 | 2338 | 1586 | 294 | 9 | −83 | C |
| ATOM | 1787 | OG1 | THR | B | 63 | 5.850 | −3.542 | −0.372 | 1.00 | 17.26 | | O |
| ANISOU | 1787 | OG1 | THR | B | 63 | 2114 | 2744 | 1699 | 940 | 202 | −224 | O |
| ATOM | 1788 | CG2 | THR | B | 63 | 4.587 | −3.464 | 1.628 | 1.00 | 15.17 | | C |
| ANISOU | 1788 | CG2 | THR | B | 63 | 1438 | 2430 | 1896 | 378 | 244 | 462 | C |
| ATOM | 1789 | N | ALA | B | 64 | 6.976 | −0.028 | −0.899 | 1.00 | 16.52 | | N |
| ANISOU | 1789 | N | ALA | B | 64 | 1460 | 3216 | 1602 | −596 | −244 | −51 | N |
| ATOM | 1790 | CA | ALA | B | 64 | 8.310 | 0.305 | −1.378 | 1.00 | 19.08 | | C |
| ANISOU | 1790 | CA | ALA | B | 64 | 1920 | 3292 | 2037 | −1201 | 270 | −679 | C |
| ATOM | 1791 | C | ALA | B | 64 | 9.082 | 1.095 | −0.350 | 1.00 | 21.33 | | C |
| ANISOU | 1791 | C | ALA | B | 64 | 2414 | 3037 | 2655 | −1354 | −231 | −593 | C |
| ATOM | 1792 | O | ALA | B | 64 | 10.185 | 0.702 | 0.099 | 1.00 | 35.83 | | O |
| ANISOU | 1792 | O | ALA | B | 64 | 1221 | 7374 | 5020 | −634 | −208 | −3212 | O |
| ATOM | 1793 | CB | ALA | B | 64 | 8.185 | 1.019 | −2.721 | 1.00 | 27.57 | | C |
| ANISOU | 1793 | CB | ALA | B | 64 | 3579 | 4569 | 2327 | −2957 | −113 | 16 | C |
| ATOM | 1794 | N | GLY | B | 65 | 8.544 | 2.238 | 0.036 | 1.00 | 29.86 | | N |
| ANISOU | 1794 | N | GLY | B | 65 | 7129 | 1490 | 2728 | −407 | −2273 | 377 | N |
| ATOM | 1795 | CA | GLY | B | 65 | 9.228 | 3.050 | 1.018 | 1.00 | 29.39 | | C |
| ANISOU | 1795 | CA | GLY | B | 65 | 6816 | 2113 | 2240 | −265 | −1929 | 199 | C |
| ATOM | 1796 | C | GLY | B | 65 | 10.646 | 3.476 | 0.744 | 1.00 | 30.36 | | C |
| ANISOU | 1796 | C | GLY | B | 65 | 7383 | 2119 | 2033 | −1063 | −1474 | 112 | C |
| ATOM | 1797 | O | GLY | B | 65 | 11.113 | 3.535 | −0.376 | 1.00 | 37.89 | | O |
| ANISOU | 1797 | O | GLY | B | 65 | 9313 | 2986 | 2095 | −2411 | −1036 | 239 | O |
| ATOM | 1798 | N | GLN | B | 66 | 11.379 | 3.782 | 1.808 | 1.00 | 28.83 | | N |
| ANISOU | 1798 | N | GLN | B | 66 | 5509 | 3012 | 2433 | 1480 | −1322 | −1161 | N |
| ATOM | 1799 | CA | GLN | B | 66 | 12.763 | 4.232 | 1.811 | 1.00 | 27.02 | | C |
| ANISOU | 1799 | CA | GLN | B | 66 | 6045 | 2536 | 1687 | 756 | −1295 | −324 | C |
| ATOM | 1800 | C | GLN | B | 66 | 13.706 | 3.067 | 1.559 | 1.00 | 24.48 | | C |
| ANISOU | 1800 | C | GLN | B | 66 | 5018 | 2491 | 1793 | 53 | 489 | 185 | C |
| ATOM | 1801 | O | GLN | B | 66 | 13.742 | 2.150 | 2.390 | 1.00 | 21.65 | | O |
| ANISOU | 1801 | O | GLN | B | 66 | 4161 | 2102 | 1965 | −152 | −639 | 31 | O |
| ATOM | 1802 | CB | GLN | B | 66 | 13.025 | 4.916 | 3.173 | 1.00 | 33.67 | | C |
| ANISOU | 1802 | CB | GLN | B | 66 | 6578 | 3752 | 2463 | 1025 | −1661 | −1329 | C |
| ATOM | 1803 | CG | GLN | B | 66 | 12.558 | 6.352 | 3.277 | 1.00 | 38.61 | | C |
| ANISOU | 1803 | CG | GLN | B | 66 | 8145 | 3698 | 2826 | 1180 | −1416 | −1491 | C |
| ATOM | 1804 | CD | GLN | B | 66 | 13.263 | 7.036 | 4.444 | 1.00 | 40.38 | | C |
| ANISOU | 1804 | CD | GLN | B | 66 | 6853 | 4506 | 3983 | −234 | −651 | −2233 | C |
| ATOM | 1805 | OE1 | GLN | B | 66 | 14.468 | 7.328 | 4.389 | 1.00 | 50.70 | | O |
| ANISOU | 1805 | OE1 | GLN | B | 66 | 7576 | 6751 | 4936 | −1855 | 77 | −753 | O |
| ATOM | 1806 | NE2 | GLN | B | 66 | 12.461 | 7.278 | 5.485 | 1.00 | 27.73 | | N |
| ANISOU | 1806 | NE2 | GLN | B | 66 | 5927 | 2095 | 2516 | 1269 | −1938 | −788 | N |
| ATOM | 1807 | N | GLU | B | 67 | 14.491 | 2.978 | 0.497 | 1.00 | 32.42 | | N |
| ANISOU | 1807 | N | GLU | B | 67 | 5259 | 4215 | 2844 | −1783 | 1348 | −736 | N |
| ATOM | 1808 | CA | GLU | B | 67 | 15.399 | 1.842 | 0.300 | 1.00 | 34.75 | | C |
| ANISOU | 1808 | CA | GLU | B | 67 | 5435 | 5483 | 2286 | −1224 | 1743 | −1781 | C |
| ATOM | 1809 | C | GLU | B | 67 | 16.317 | 1.566 | 1.474 | 1.00 | 34.96 | | C |
| ANISOU | 1809 | C | GLU | B | 67 | 4256 | 5064 | 3965 | −1544 | 1054 | −1791 | C |
| ATOM | 1810 | O | GLU | B | 67 | 16.633 | 0.414 | 1.784 | 1.00 | 27.40 | | O |
| ANISOU | 1810 | O | GLU | B | 67 | 2194 | 4778 | 3438 | −1317 | 1510 | −2800 | O |
| ATOM | 1811 | CB | GLU | B | 67 | 16.298 | 2.035 | −0.934 | 1.00 | 47.53 | | C |
| ANISOU | 1811 | CB | GLU | B | 67 | 6276 | 8162 | 3622 | −2872 | 2884 | −2298 | C |
| ATOM | 1812 | CG | GLU | B | 67 | 17.783 | 1.775 | −0.690 | 1.00 | 55.01 | | C |
| ANISOU | 1812 | CG | GLU | B | 67 | 6097 | 9808 | 4997 | −2838 | 3189 | −1997 | C |
| ATOM | 1813 | CD | GLU | B | 67 | 18.319 | 0.368 | −0.848 | 1.00 | 64.81 | | C |
| ANISOU | 1813 | CD | GLU | B | 67 | 6909 | 10341 | 7377 | −1682 | 846 | −2052 | C |
| ATOM | 1814 | OE1 | GLU | B | 67 | 18.233 | −0.408 | 0.140 | 1.00 | 74.26 | | O |
| ANISOU | 1814 | OE1 | GLU | B | 67 | 9794 | 10896 | 7526 | −1732 | −1617 | −1362 | O |
| ATOM | 1815 | OE2 | GLU | B | 67 | 18.850 | 0.005 | −1.929 | 1.00 | 63.12 | | O |
| ANISOU | 1815 | OE2 | GLU | B | 67 | 5059 | 11355 | 7569 | 923 | −759 | −2901 | O |
| ATOM | 1816 | N | ARG | B | 68 | 16.807 | 2.593 | 2.203 | 1.00 | 28.79 | | N |
| ANISOU | 1816 | N | ARG | B | 68 | 3278 | 4171 | 3489 | −927 | 1538 | −1444 | N |
| ATOM | 1817 | CA | ARG | B | 68 | 17.755 | 2.218 | 3.257 | 1.00 | 28.49 | | C |
| ANISOU | 1817 | CA | ARG | B | 68 | 1777 | 4575 | 4475 | −916 | 1516 | −1724 | C |
| ATOM | 1818 | C | ARG | B | 68 | 17.059 | 1.478 | 4.381 | 1.00 | 23.19 | | C |
| ANISOU | 1818 | C | ARG | B | 68 | 1187 | 3498 | 4126 | −70 | 753 | −1255 | C |
| ATOM | 1819 | O | ARG | B | 68 | 17.704 | 0.854 | 5.213 | 1.00 | 24.52 | | O |
| ANISOU | 1819 | O | ARG | B | 68 | 1029 | 3866 | 4420 | 130 | −46 | −1940 | O |
| ATOM | 1820 | CB | ARG | B | 68 | 18.466 | 3.433 | 3.842 | 1.00 | 30.20 | | C |
| ANISOU | 1820 | CB | ARG | B | 68 | 1627 | 4525 | 5323 | −951 | 1642 | −1824 | C |
| ATOM | 1821 | CG | ARG | B | 68 | 17.507 | 4.432 | 4.412 | 1.00 | 29.51 | | C |
| ANISOU | 1821 | CG | ARG | B | 68 | 2013 | 3848 | 5352 | −466 | 1195 | −1218 | C |
| ATOM | 1822 | CD | ARG | B | 68 | 18.230 | 5.674 | 4.845 | 1.00 | 31.72 | | C |
| ANISOU | 1822 | CD | ARG | B | 68 | 3524 | 3562 | 4965 | −735 | 656 | −729 | C |
| ATOM | 1823 | NE | ARG | B | 68 | 18.756 | 5.598 | 6.220 | 1.00 | 29.73 | | N |
| ANISOU | 1823 | NE | ARG | B | 68 | 3411 | 2864 | 5022 | −873 | 631 | −232 | N |
| ATOM | 1824 | CZ | ARG | B | 68 | 19.423 | 6.631 | 6.730 | 1.00 | 27.73 | | C |
| ANISOU | 1824 | CZ | ARG | B | 68 | 2636 | 2430 | 5471 | −226 | −137 | 72 | C |
| ATOM | 1825 | NH1 | ARG | B | 68 | 19.595 | 7.689 | 5.950 | 1.00 | 40.60 | | N |
| ANISOU | 1825 | NH1 | ARG | B | 68 | 5051 | 3548 | 6830 | −1924 | −1295 | 1176 | N |
| ATOM | 1826 | NH2 | ARG | B | 68 | 19.904 | 6.603 | 7.958 | 1.00 | 40.65 | | N |
| ANISOU | 1826 | NH2 | ARG | B | 68 | 6693 | 3574 | 5178 | −1242 | −557 | −182 | N |
| ATOM | 1827 | N | PHE | B | 69 | 15.735 | 1.565 | 4.399 | 1.00 | 15.74 | | N |
| ANISOU | 1827 | N | PHE | B | 69 | 1196 | 2201 | 2584 | −256 | 331 | −920 | N |
| ATOM | 1828 | CA | PHE | B | 69 | 14.952 | 0.832 | 5.395 | 1.00 | 13.95 | | C |
| ANISOU | 1828 | CA | PHE | B | 69 | 1080 | 2119 | 2103 | −37 | 22 | −896 | C |
| ATOM | 1829 | C | PHE | B | 69 | 14.231 | −0.384 | 4.841 | 1.00 | 13.10 | | C |
| ANISOU | 1829 | C | PHE | B | 69 | 1273 | 1900 | 1805 | −59 | −21 | −594 | C |
| ATOM | 1830 | O | PHE | B | 69 | 13.261 | −0.839 | 5.442 | 1.00 | 12.55 | | O |
| ANISOU | 1830 | O | PHE | B | 69 | 1336 | 1647 | 1783 | 101 | 35 | −239 | O |
| ATOM | 1831 | CB | PHE | B | 69 | 13.986 | 1.828 | 6.039 | 1.00 | 13.33 | | C |
| ANISOU | 1831 | CB | PHE | B | 69 | 1050 | 2033 | 1980 | 209 | −120 | −683 | C |
| ATOM | 1832 | CG | PHE | B | 69 | 14.679 | 2.932 | 6.825 | 1.00 | 13.37 | | C |
| ANISOU | 1832 | CG | PHE | B | 69 | 1061 | 2122 | 1897 | 174 | 36 | −776 | C |
| ATOM | 1833 | CD1 | PHE | B | 69 | 14.601 | 4.262 | 6.438 | 1.00 | 13.62 | | C |
| ANISOU | 1833 | CD1 | PHE | B | 69 | 1267 | 2062 | 1846 | 181 | 230 | −810 | C |
| ATOM | 1834 | CD2 | PHE | B | 69 | 15.404 | 2.601 | 7.958 | 1.00 | 14.40 | | C |
| ANISOU | 1834 | CD2 | PHE | B | 69 | 1481 | 2201 | 1790 | 490 | −35 | −1006 | C |
| ATOM | 1835 | CE1 | PHE | B | 69 | 15.235 | 5.244 | 7.189 | 1.00 | 17.03 | | C |
| ANISOU | 1835 | CE1 | PHE | B | 69 | 1486 | 2209 | 2777 | −219 | −462 | −509 | C |
| ATOM | 1836 | CE2 | PHE | B | 69 | 16.003 | 3.582 | 8.727 | 1.00 | 17.70 | | C |
| ANISOU | 1836 | CE2 | PHE | B | 69 | 1770 | 2399 | 2556 | 95 | −731 | −728 | C |
| ATOM | 1837 | CZ | PHE | B | 69 | 15.911 | 4.905 | 8.354 | 1.00 | 15.75 | | C |
| ANISOU | 1837 | CZ | PHE | B | 69 | 1099 | 2312 | 2572 | 39 | −234 | −770 | C |
| ATOM | 1838 | N | ARG | B | 70 | 14.710 | −0.905 | 3.710 | 1.00 | 12.71 | | N |
| ANISOU | 1838 | N | ARG | B | 70 | 916 | 2127 | 1788 | 75 | −129 | −638 | N |
| ATOM | 1839 | CA | ARG | B | 70 | 14.051 | −2.047 | 3.114 | 1.00 | 12.69 | | C |
| ANISOU | 1839 | CA | ARG | B | 70 | 1122 | 2033 | 1668 | 24 | −154 | −601 | C |
| ATOM | 1840 | C | ARG | B | 70 | 13.852 | −3.196 | 4.081 | 1.00 | 13.43 | | C |
| ANISOU | 1840 | C | ARG | B | 70 | 1061 | 2159 | 1884 | 60 | −209 | −446 | C |
| ATOM | 1841 | O | ARG | B | 70 | 12.835 | −3.898 | 4.004 | 1.00 | 13.78 | | O |
| ANISOU | 1841 | O | ARG | B | 70 | 1102 | 2200 | 1934 | −31 | 113 | −629 | O |
| ATOM | 1842 | CB | ARG | B | 70 | 14.819 | −2.540 | 1.857 | 1.00 | 14.96 | | C |
| ANISOU | 1842 | CB | ARG | B | 70 | 1209 | 2647 | 1828 | −233 | −5 | −902 | C |
| ATOM | 1843 | CG | ARG | B | 70 | 16.232 | −3.003 | 2.156 | 1.00 | 17.53 | | C |
| ANISOU | 1843 | CG | ARG | B | 70 | 1202 | 3259 | 2200 | −6 | 207 | −951 | C |
| ATOM | 1844 | CD | ARG | B | 70 | 17.047 | −3.323 | 0.914 | 1.00 | 14.53 | | C |
| ANISOU | 1844 | CD | ARG | B | 70 | 1324 | 2007 | 2192 | −224 | 289 | −643 | C |
| ATOM | 1845 | NE | ARG | B | 70 | 18.389 | −3.745 | 1.327 | 1.00 | 18.12 | | N |
| ANISOU | 1845 | NE | ARG | B | 70 | 1220 | 2726 | 2940 | −27 | 399 | −735 | N |
| ATOM | 1846 | CZ | ARG | B | 70 | 19.290 | −4.218 | 0.453 | 1.00 | 24.74 | | C |
| ANISOU | 1846 | CZ | ARG | B | 70 | 1796 | 4008 | 3595 | 621 | 498 | −1343 | C |
| ATOM | 1847 | NH1 | ARG | B | 70 | 18.957 | −4.315 | −0.826 | 1.00 | 22.47 | | N |
| ANISOU | 1847 | NH1 | ARG | B | 70 | 1832 | 3316 | 3389 | −958 | 1035 | −1422 | N |
| ATOM | 1848 | NH2 | ARG | B | 70 | 20.493 | −4.587 | 0.863 | 1.00 | 23.87 | | N |
| ANISOU | 1848 | NH2 | ARG | B | 70 | 1234 | 2858 | 4978 | −123 | 602 | −1223 | N |
| ATOM | 1849 | N | SER | B | 71 | 14.742 | −3.487 | 5.020 | 1.00 | 14.81 | | N |
| ANISOU | 1849 | N | SER | B | 71 | 1696 | 2258 | 1674 | 206 | −478 | −742 | N |
| ATOM | 1850 | CA | SER | B | 71 | 14.607 | −4.579 | 5.957 | 1.00 | 16.80 | | C |
| ANISOU | 1850 | CA | SER | B | 71 | 1689 | 2286 | 2407 | 689 | −388 | −317 | C |
| ATOM | 1851 | C | SER | B | 71 | 13.439 | −4.385 | 6.906 | 1.00 | 15.20 | | C |
| ANISOU | 1851 | C | SER | B | 71 | 1677 | 1904 | 2194 | 239 | −423 | −423 | C |
| ATOM | 1852 | O | SER | B | 71 | 12.947 | −5.338 | 7.536 | 1.00 | 16.97 | | O |
| ANISOU | 1852 | O | SER | B | 71 | 1917 | 1945 | 2585 | 24 | −509 | −339 | O |
| ATOM | 1853 | CB | SER | B | 71 | 15.911 | −4.703 | 6.779 | 1.00 | 20.77 | | C |
| ANISOU | 1853 | CB | SER | B | 71 | 1890 | 3269 | 2732 | 1134 | −646 | −377 | C |
| ATOM | 1854 | OG | SER | B | 71 | 16.109 | −3.562 | 7.635 | 1.00 | 27.30 | | O |
| ANISOU | 1854 | OG | SER | B | 71 | 2411 | 4386 | 3577 | −430 | −517 | −1057 | O |
| ATOM | 1855 | N | LEU | B | 72 | 12.956 | −3.166 | 7.058 | 1.00 | 13.60 | | N |
| ANISOU | 1855 | N | LEU | B | 72 | 1248 | 1924 | 1993 | 150 | −518 | −431 | N |
| ATOM | 1856 | CA | LEU | B | 72 | 11.808 | −2.864 | 7.908 | 1.00 | 15.29 | | C |
| ANISOU | 1856 | CA | LEU | B | 72 | 1232 | 2393 | 2183 | 0 | −511 | −898 | C |
| ATOM | 1857 | C | LEU | B | 72 | 10.517 | −2.940 | 7.128 | 1.00 | 12.48 | | C |
| ANISOU | 1857 | C | LEU | B | 72 | 1122 | 2020 | 1599 | −507 | −166 | −228 | C |
| ATOM | 1858 | O | LEU | B | 72 | 9.438 | −2.873 | 7.774 | 1.00 | 13.85 | | O |
| ANISOU | 1858 | O | LEU | B | 72 | 1155 | 2289 | 1818 | −371 | −30 | −453 | O |
| ATOM | 1859 | CB | LEU | B | 72 | 11.958 | −1.459 | 8.490 | 1.00 | 12.44 | | C |
| ANISOU | 1859 | CB | LEU | B | 72 | 821 | 2187 | 1717 | −280 | −72 | −457 | C |
| ATOM | 1860 | CG | LEU | B | 72 | 13.192 | −1.264 | 9.366 | 1.00 | 14.51 | | C |
| ANISOU | 1860 | CG | LEU | B | 72 | 1299 | 2168 | 2046 | −96 | −492 | −712 | C |
| ATOM | 1861 | CD1 | LEU | B | 72 | 13.189 | 0.141 | 9.919 | 1.00 | 15.77 | | C |
| ANISOU | 1861 | CD1 | LEU | B | 72 | 1673 | 2326 | 1992 | −442 | 156 | −969 | C |
| ATOM | 1862 | CD2 | LEU | B | 72 | 13.227 | −2.278 | 10.514 | 1.00 | 26.36 | | C |
| ANISOU | 1862 | CD2 | LEU | B | 72 | 4544 | 2684 | 2787 | −528 | −2128 | −152 | C |
| ATOM | 1863 | N | ARG | B | 73 | 10.554 | −3.062 | 5.813 | 1.00 | 13.58 | | N |
| ANISOU | 1863 | N | ARG | B | 73 | 1120 | 2437 | 1605 | −402 | −141 | −67 | N |
| ATOM | 1864 | CA | ARG | B | 73 | 9.332 | −3.130 | 5.037 | 1.00 | 12.41 | | C |
| ANISOU | 1864 | CA | ARG | B | 73 | 1324 | 1645 | 1748 | 180 | −470 | 11 | C |
| ATOM | 1865 | C | ARG | B | 73 | 8.916 | −4.577 | 4.782 | 1.00 | 10.94 | | C |
| ANISOU | 1865 | C | ARG | B | 73 | 936 | 1863 | 1356 | 72 | 94 | −429 | C |
| ATOM | 1866 | O | ARG | B | 73 | 7.701 | −4.855 | 4.689 | 1.00 | 11.72 | | O |
| ANISOU | 1866 | O | ARG | B | 73 | 948 | 2199 | 1305 | 27 | 27 | −147 | O |
| ATOM | 1867 | CB | ARG | B | 73 | 9.484 | −2.462 | 3.664 | 1.00 | 15.30 | | C |
| ANISOU | 1867 | CB | ARG | B | 73 | 1750 | 2217 | 1847 | 292 | −99 | 224 | C |
| ATOM | 1868 | CG | ARG | B | 73 | 10.086 | −3.353 | 2.552 | 1.00 | 26.09 | | C |
| ANISOU | 1868 | CG | ARG | B | 73 | 3128 | 4331 | 2454 | 494 | 748 | −572 | C |
| ATOM | 1869 | CD | ARG | B | 73 | 10.333 | −2.665 | 1.221 | 1.00 | 24.82 | | C |
| ANISOU | 1869 | CD | ARG | B | 73 | 2660 | 4440 | 2332 | −199 | 521 | −736 | C |
| ATOM | 1870 | NE | ARG | B | 73 | 11.058 | −3.467 | 0.230 | 1.00 | 19.67 | | N |
| ANISOU | 1870 | NE | ARG | B | 73 | 2463 | 2907 | 2103 | −503 | 338 | −233 | N |
| ATOM | 1871 | CZ | ARG | B | 73 | 11.726 | −3.043 | −0.839 | 1.00 | 20.11 | | C |
| ANISOU | 1871 | CZ | AEG | B | 73 | 2772 | 1809 | 3060 | −619 | 1164 | −406 | C |
| ATOM | 1872 | NH1 | ARG | B | 73 | 11.776 | −1.732 | −1.080 | 1.00 | 26.50 | | N |
| ANISOU | 1872 | NH1 | ARG | B | 73 | 4225 | 1784 | 4059 | 64 | 973 | −39 | N |
| ATOM | 1873 | NH2 | ARG | B | 73 | 12.348 | −3.891 | −1.664 | 1.00 | 15.79 | | N |
| ANISOU | 1873 | NH2 | ARG | B | 73 | 1968 | 1892 | 2138 | −31 | 65 | −115 | N |
| ATOM | 1874 | N | THR | B | 74 | 9.855 | −5.500 | 4.638 | 1.00 | 13.76 | | N |
| ANISOU | 1874 | N | THR | B | 74 | 1080 | 1860 | 2288 | 135 | −291 | −600 | N |
| ATOM | 1875 | CA | THR | B | 74 | 9.488 | −6.884 | 4.316 | 1.00 | 13.31 | | C |
| ANISOU | 1875 | CA | THR | B | 74 | 1232 | 1733 | 2093 | 124 | −222 | −337 | C |
| ATOM | 1876 | C | THR | B | 74 | 8.639 | −7.552 | 5.392 | 1.00 | 13.55 | | C |
| ANISOU | 1876 | C | THR | B | 74 | 1035 | 2284 | 1832 | −102 | −324 | −483 | C |
| ATOM | 1877 | O | THR | B | 74 | 7.871 | −8.487 | 4.975 | 1.00 | 13.80 | | O |
| ANISOU | 1877 | O | THR | B | 74 | 1144 | 2133 | 1968 | −52 | −157 | −699 | O |
| ATOM | 1878 | CB | THR | B | 74 | 10.745 | −7.720 | 4.028 | 1.00 | 15.53 | | C |
| ANISOU | 1878 | CB | THR | B | 74 | 1099 | 2066 | 2736 | −109 | −34 | −1229 | C |
| ATOM | 1879 | OG1 | THR | B | 74 | 11.669 | −7.636 | 5.119 | 1.00 | 18.66 | | O |
| ANISOU | 1879 | OG1 | THR | B | 74 | 1381 | 2528 | 3183 | 309 | −571 | −499 | O |
| ATOM | 1880 | CG2 | THR | B | 74 | 11.448 | −7.210 | 2.810 | 1.00 | 15.23 | | C |
| ANISOU | 1880 | CG2 | THR | B | 74 | 1114 | 1683 | 2990 | 207 | −78 | −751 | C |
| ATOM | 1881 | N | PRO | B | 75 | 8.646 | −7.229 | 6.687 | 1.00 | 13.29 | | N |
| ANISOU | 1881 | N | PRO | B | 75 | 1137 | 2062 | 1853 | 65 | −431 | −483 | N |
| ATOM | 1882 | CA | PRO | B | 75 | 7.668 | −7.906 | 7.548 | 1.00 | 12.75 | | C |
| ANISOU | 1882 | CA | PRO | B | 75 | 1490 | 1760 | 1594 | 111 | −540 | −228 | C |
| ATOM | 1883 | C | PRO | B | 75 | 6.214 | −7.747 | 7.124 | 1.00 | 12.97 | | C |
| ANISOU | 1883 | C | PRO | B | 75 | 1298 | 2046 | 1585 | −96 | −465 | 74 | C |
| ATOM | 1884 | O | PRO | B | 75 | 5.304 | −8.562 | 7.395 | 1.00 | 15.83 | | O |
| ANISOU | 1884 | O | PRO | B | 75 | 1727 | 2495 | 1793 | −355 | −187 | 255 | O |
| ATOM | 1885 | CB | PRO | B | 75 | 7.887 | −7.215 | 8.900 | 1.00 | 15.51 | | C |
| ANISOU | 1885 | CB | PRO | B | 75 | 1840 | 2409 | 1645 | −634 | −599 | −323 | C |
| ATOM | 1886 | CG | PRO | B | 75 | 9.354 | −6.853 | 8.882 | 1.00 | 15.37 | | C |
| ANISOU | 1886 | CG | PRO | B | 75 | 1706 | 2383 | 1751 | −352 | −407 | −428 | C |
| ATOM | 1887 | CD | PRO | B | 75 | 9.554 | −6.377 | 7.475 | 1.00 | 12.97 | | C |
| ANISOU | 1887 | CD | PRO | B | 75 | 1374 | 1745 | 1810 | 78 | −588 | −211 | C |
| ATOM | 1888 | N | PHE | B | 76 | 5.953 | −6.671 | 6.381 | 1.00 | 12.24 | | N |
| ANISOU | 1888 | N | PHE | B | 76 | 1216 | 1879 | 1555 | −40 | −519 | −103 | N |
| ATOM | 1889 | CA | PHE | B | 76 | 4.587 | −6.370 | 5.921 | 1.00 | 10.81 | | C |
| ANISOU | 1889 | CA | PHE | B | 76 | 932 | 1928 | 1246 | −7 | −159 | −249 | C |
| ATOM | 1890 | C | PHE | B | 76 | 4.234 | −7.130 | 4.645 | 1.00 | 10.72 | | C |
| ANISOU | 1890 | C | PHE | B | 76 | 1026 | 1958 | 1090 | −88 | −134 | −178 | C |
| ATOM | 1891 | O | PHE | B | 76 | 3.070 | −7.044 | 4.203 | 1.00 | 12.69 | | O |
| ANISOU | 1891 | O | PHE | B | 76 | 1004 | 2540 | 1279 | −118 | −217 | −350 | O |
| ATOM | 1892 | CB | PHE | B | 76 | 4.439 | −4.864 | 5.726 | 1.00 | 12.20 | | C |
| ANISOU | 1892 | CB | PHE | B | 76 | 1352 | 1935 | 1350 | 304 | −27 | −242 | C |
| ATOM | 1893 | CG | PHE | B | 76 | 4.618 | −4.185 | 7.089 | 1.00 | 12.12 | | C |
| ANISOU | 1893 | CG | PHE | B | 76 | 1262 | 1909 | 1432 | 207 | −79 | −256 | C |
| ATOM | 1894 | CD1 | PHE | B | 76 | 3.590 | −4.165 | 8.008 | 1.00 | 14.47 | | C |
| ANISOU | 1894 | CD1 | PHE | B | 76 | 1382 | 2450 | 1667 | 250 | 122 | −743 | C |
| ATOM | 1895 | CD2 | PHE | B | 76 | 5.818 | −3.616 | 7.440 | 1.00 | 14.12 | | C |
| ANISOU | 1895 | CD2 | PHE | B | 76 | 1464 | 1739 | 2163 | 9 | 23 | −589 | C |
| ATOM | 1896 | CE1 | PHE | B | 76 | 3.762 | −3.616 | 9.262 | 1.00 | 14.37 | | C |
| ANISOU | 1896 | CE1 | PHE | B | 76 | 1984 | 1842 | 1633 | 296 | 32 | −533 | C |
| ATOM | 1897 | CE2 | PHE | B | 76 | 6.005 | −3.099 | 8.687 | 1.00 | 15.08 | | C |
| ANISOU | 1897 | CE2 | PHE | B | 76 | 1588 | 2143 | 1998 | 351 | −660 | −303 | C |
| ATOM | 1898 | CZ | PHE | B | 76 | 4.991 | −3.103 | 9.616 | 1.00 | 15.90 | | C |
| ANISOU | 1898 | CZ | PHE | B | 76 | 2242 | 2134 | 1665 | 216 | −432 | −373 | C |
| ATOM | 1899 | N | TYR | B | 77 | 5.151 | −7.887 | 4.075 | 1.00 | 10.60 | | N |
| ANISOU | 1899 | N | TYR | B | 77 | 800 | 2021 | 1206 | −401 | −30 | −335 | N |
| ATOM | 1900 | CA | TYR | B | 77 | 4.754 | −8.802 | 3.001 | 1.00 | 9.69 | | C |
| ANISOU | 1900 | CA | TYR | B | 77 | 973 | 1609 | 1101 | −103 | −182 | −111 | C |
| ATOM | 1901 | C | TYR | B | 77 | 3.898 | −9.936 | 3.529 | 1.00 | 9.76 | | C |
| ANISOU | 1901 | C | TYR | B | 77 | 850 | 1773 | 1085 | −233 | −236 | −65 | C |
| ATOM | 1902 | O | TYR | B | 77 | 3.042 | −10.444 | 2.838 | 1.00 | 9.66 | | O |
| ANISOU | 1902 | O | TYR | B | 77 | 863 | 1693 | 1112 | −117 | −339 | −122 | O |
| ATOM | 1903 | CB | TYR | B | 77 | 5.979 | −9.410 | 2.317 | 1.00 | 9.85 | | C |
| ANISOU | 1903 | CB | TYR | B | 77 | 937 | 1421 | 1385 | −57 | −41 | 38 | C |
| ATOM | 1904 | CG | TYR | B | 77 | 6.875 | −8.514 | 1.504 | 1.00 | 9.42 | | C |
| ANISOU | 1904 | CG | TYR | B | 77 | 1253 | 1322 | 1002 | −178 | −43 | −167 | C |
| ATOM | 1905 | CD1 | TYR | B | 77 | 6.715 | −7.133 | 1.485 | 1.00 | 12.38 | | C |
| ANISOU | 1905 | CD1 | TYR | B | 77 | 1468 | 1329 | 1906 | −190 | 395 | −185 | C |
| ATOM | 1906 | CD2 | TYR | B | 77 | 7.881 | −9.035 | 0.736 | 1.00 | 9.47 | | C |
| ANISOU | 1906 | CD2 | TYR | B | 77 | 781 | 1444 | 1374 | −198 | −134 | −145 | C |
| ATOM | 1907 | CE1 | TYR | B | 77 | 7.519 | −6.304 | 0.740 | 1.00 | 11.27 | | C |
| ANISOU | 1907 | CE1 | TYR | B | 77 | 1272 | 1301 | 1710 | 121 | 189 | 60 | C |
| ATOM | 1908 | CE2 | TYR | B | 77 | 8.716 | −8.237 | −0.024 | 1.00 | 10.55 | | C |
| ANISOU | 1908 | CE2 | TYR | B | 77 | 768 | 1336 | 1904 | −179 | 81 | −179 | C |
| ATOM | 1909 | CZ | TYR | B | 77 | 8.548 | −6.861 | −0.008 | 1.00 | 10.99 | | C |
| ANISOU | 1909 | CZ | TYR | B | 77 | 944 | 1398 | 1833 | 12 | 118 | −26 | C |
| ATOM | 1910 | OH | TYR | B | 77 | 9.320 | −6.005 | −0.745 | 1.00 | 12.51 | | O |
| ANISOU | 1910 | OH | TYR | B | 77 | 1421 | 1457 | 1873 | 92 | 356 | 93 | O |
| ATOM | 1911 | N | ARG | B | 78 | 4.175 | −10.436 | 4.741 | 1.00 | 15.11 | | N |
| ANISOU | 1911 | N | ARG | B | 78 | 1094 | 3168 | 1478 | −1104 | −771 | 593 | N |
| ATOM | 1912 | CA | ARG | B | 78 | 3.473 | −11.566 | 5.324 | 1.00 | 15.08 | | C |
| ANISOU | 1912 | CA | ARG | B | 78 | 1156 | 2995 | 1579 | −862 | −601 | 689 | C |
| ATOM | 1913 | C | ARG | B | 78 | 1.965 | −11.315 | 5.402 | 1.00 | 14.48 | | C |
| ANISOU | 1913 | C | ARG | B | 78 | 1162 | 2703 | 1637 | −795 | −413 | 655 | C |
| ATOM | 1914 | O | ARG | B | 78 | 1.561 | −10.199 | 5.683 | 1.00 | 15.60 | | O |
| ANISOU | 1914 | O | ARG | B | 78 | 1738 | 2967 | 1223 | −621 | −281 | 306 | O |
| ATOM | 1915 | CB | ARG | B | 78 | 3.934 | −11.864 | 6.738 | 1.00 | 21.46 | | C |
| ANISOU | 1915 | CB | ARG | B | 78 | 1908 | 4222 | 2023 | −1150 | −1156 | 1364 | C |
| ATOM | 1916 | CG | ARG | B | 78 | 5.257 | −12.595 | 6.756 | 1.00 | 36.73 | | C |
| ANISOU | 1916 | CG | ARG | B | 78 | 4213 | 6369 | 3373 | 1492 | −2770 | −458 | C |
| ATOM | 1917 | CD | ARG | B | 78 | 5.323 | −13.444 | 8.041 | 1.00 | 49.51 | | C |
| ANISOU | 1917 | CD | ARG | B | 78 | 6792 | 7357 | 4663 | 1564 | −4671 | 692 | C |
| ATOM | 1918 | NE | ARG | B | 78 | 6.470 | −14.333 | 7.852 | 1.00 | 54.62 | | N |
| ANISOU | 1918 | NE | ARG | B | 78 | 6217 | 8910 | 5624 | 1902 | −4562 | 1307 | N |
| ATOM | 1919 | CZ | ARG | B | 78 | 7.718 | −13.864 | 7.957 | 1.00 | 63.81 | | C |
| ANISOU | 1919 | CZ | ARG | B | 78 | 6571 | 10420 | 7255 | 1100 | −4258 | 475 | C |
| ATOM | 1920 | NH1 | ARG | B | 78 | 7.964 | −12.582 | 8.243 | 1.00 | 57.28 | | N |
| ANISOU | 1920 | NH1 | ARG | B | 78 | 8288 | 9133 | 4343 | −685 | −1076 | 4452 | N |
| ATOM | 1921 | NH2 | ARG | B | 78 | 8.718 | −14.719 | 7.772 | 1.00 | 71.79 | | N |
| ANISOU | 1921 | NH2 | ARG | B | 78 | 5947 | 12314 | 9015 | 886 | −1723 | −477 | N |
| ATOM | 1922 | N | GLY | B | 79 | 1.207 | −12.374 | 5.118 | 1.00 | 14.95 | | N |
| ANISOU | 1922 | N | GLY | B | 79 | 1255 | 2789 | 1637 | −957 | −421 | 835 | N |
| ATOM | 1923 | CA | GLY | B | 79 | −0.249 | −12.258 | 5.174 | 1.00 | 15.82 | | C |
| ANISOU | 1923 | CA | GLY | B | 79 | 1262 | 3217 | 1531 | −984 | −490 | 437 | C |
| ATOM | 1924 | C | GLY | B | 79 | −0.852 | −11.819 | 3.856 | 1.00 | 13.09 | | C |
| ANISOU | 1924 | C | GLY | B | 79 | 1162 | 2494 | 1316 | −412 | −225 | 80 | C |
| ATOM | 1925 | O | GLY | B | 79 | −2.066 | −11.805 | 3.725 | 1.00 | 13.32 | | O |
| ANISOU | 1925 | O | GLY | B | 79 | 1027 | 2324 | 1711 | −316 | −188 | 60 | O |
| ATOM | 1926 | N | SER | B | 80 | −0.026 | −11.437 | 2.878 | 1.00 | 11.74 | | N |
| ANISOU | 1926 | N | SER | B | 80 | 1044 | 2203 | 1213 | −246 | −233 | −166 | N |
| ATOM | 1927 | CA | SER | B | 80 | −0.575 | −11.055 | 1.587 | 1.00 | 10.22 | | C |
| ANISOU | 1927 | CA | SER | B | 80 | 999 | 1659 | 1225 | −80 | −281 | −186 | C |
| ATOM | 1928 | C | SER | B | 80 | −1.273 | −12.237 | 0.967 | 1.00 | 10.24 | | C |
| ANISOU | 1928 | C | SER | B | 80 | 997 | 1596 | 1299 | −26 | −235 | −176 | C |
| ATOM | 1929 | O | SER | B | 80 | −0.766 | −13.351 | 1.015 | 1.00 | 15.05 | | O |
| ANISOU | 1929 | O | SER | B | 80 | 1601 | 1649 | 2468 | 149 | −927 | −400 | O |
| ATOM | 1930 | CB | SER | B | 80 | 0.558 | −10.559 | 0.689 | 1.00 | 10.25 | | C |
| ANISOU | 1930 | CB | SER | B | 80 | 1055 | 1604 | 1236 | −61 | −280 | −155 | C |
| ATOM | 1931 | OG | SER | B | 80 | 1.133 | −9.362 | 1.201 | 1.00 | 12.34 | | O |
| ANISOU | 1931 | OG | SER | B | 80 | 1362 | 1964 | 1363 | −454 | −464 | −143 | O |
| ATOM | 1932 | N | ASP | B | 81 | −2.415 | −12.006 | 0.354 | 1.00 | 9.96 | | N |
| ANISOU | 1932 | N | ASP | B | 81 | 912 | 1472 | 1399 | −115 | −264 | −286 | N |
| ATOM | 1933 | CA | ASP | B | 81 | −3.187 | −13.017 | −0.356 | 1.00 | 10.10 | | C |
| ANISOU | 1933 | CA | ASP | B | 81 | 1051 | 1454 | 1334 | −410 | −224 | 64 | C |
| ATOM | 1934 | C | ASP | B | 81 | −2.902 | −13.071 | −1.841 | 1.00 | 9.80 | | C |
| ANISOU | 1934 | C | ASP | B | 81 | 919 | 1541 | 1263 | −517 | −318 | −55 | C |
| ATOM | 1935 | O | ASP | B | 81 | −3.123 | −14.110 | −2.465 | 1.00 | 10.26 | | O |
| ANISOU | 1935 | O | ASP | B | 81 | 1069 | 1323 | 1507 | −151 | −373 | −48 | O |
| ATOM | 1936 | CB | ASP | B | 81 | −4.682 | −12.717 | −0.117 | 1.00 | 11.48 | | C |
| ANISOU | 1936 | CB | ASP | B | 81 | 1008 | 1863 | 1492 | −693 | −18 | 180 | C |
| ATOM | 1937 | CG | ASP | B | 81 | −5.015 | −12.834 | 1.378 | 1.00 | 12.92 | | C |
| ANISOU | 1937 | CG | ASP | B | 81 | 1730 | 1532 | 1645 | −440 | 348 | 384 | C |
| ATOM | 1938 | OD1 | ASP | B | 81 | −4.733 | −13.871 | 2.009 | 1.00 | 16.56 | | O |
| ANISOU | 1938 | OD1 | ASP | B | 81 | 2075 | 2128 | 2091 | −55 | 105 | 813 | O |
| ATOM | 1939 | OD2 | ASP | B | 81 | −5.610 | −11.886 | 1.921 | 1.00 | 14.27 | | O |
| ANISOU | 1939 | OD2 | ASP | B | 81 | 1460 | 2059 | 1902 | −218 | 212 | 56 | O |
| ATOM | 1940 | N | CYS | B | 82 | −2.473 | −11.990 | −2.464 | 1.00 | 9.22 | | N |
| ANISOU | 1940 | N | CYS | B | 82 | 858 | 1428 | 1215 | −433 | −28 | −306 | N |
| ATOM | 1941 | CA | CYS | B | 82 | −2.119 | −11.892 | −3.874 | 1.00 | 9.41 | | C |
| ANISOU | 1941 | CA | CYS | B | 82 | 817 | 1529 | 1228 | −264 | 13 | −208 | C |
| ATOM | 1942 | C | CYS | B | 82 | −1.062 | −10.820 | −4.025 | 1.00 | 9.09 | | C |
| ANISOU | 1942 | C | CYS | B | 82 | 848 | 1364 | 1242 | −115 | 161 | −210 | C |
| ATOM | 1943 | O | CYS | B | 82 | −1.093 | −9.857 | −3.256 | 1.00 | 9.95 | | O |
| ANISOU | 1943 | O | CYS | B | 82 | 881 | 1400 | 1500 | −161 | 220 | −305 | O |
| ATOM | 1944 | CB | CYS | B | 82 | −3.356 | −11.527 | −4.698 | 1.00 | 10.33 | | C |
| ANISOU | 1944 | CB | CYS | B | 82 | 993 | 1540 | 1392 | −149 | −90 | −177 | C |
| ATOM | 1945 | SG | CYS | B | 82 | −3.152 | −11.467 | −6.487 | 1.00 | 13.15 | | S |
| ANISOU | 1945 | SG | CYS | B | 82 | 1278 | 2287 | 1433 | −43 | −396 | −135 | S |
| ATOM | 1946 | N | CYS | B | 83 | −0.185 | −10.993 | −4.986 | 1.00 | 8.91 | | N |
| ANISOU | 1946 | N | CYS | B | 83 | 664 | 1588 | 1133 | −39 | 31 | −202 | N |
| ATOM | 1947 | CA | CYS | B | 83 | 0.858 | −10.016 | −5.274 | 1.00 | 10.47 | | C |
| ANISOU | 1947 | CA | CYS | B | 83 | 927 | 1904 | 1147 | −170 | 136 | −123 | C |
| ATOM | 1948 | C | CYS | B | 83 | 0.697 | −9.527 | −6.710 | 1.00 | 9.65 | | C |
| ANISOU | 1948 | C | CYS | B | 83 | 965 | 1652 | 1048 | −409 | 106 | −316 | C |
| ATOM | 1949 | O | CYS | B | 83 | 0.651 | −10.356 | −7.639 | 1.00 | 10.26 | | O |
| ANISOU | 1949 | O | CYS | B | 83 | 1340 | 1315 | 1243 | −421 | −61 | −237 | O |
| ATOM | 1950 | CB | CYS | B | 83 | 2.209 | −10.670 | −5.039 | 1.00 | 11.99 | | C |
| ANISOU | 1950 | CB | CYS | B | 83 | 706 | 2611 | 1240 | −105 | 162 | −355 | C |
| ATOM | 1951 | SG | CYS | B | 83 | 3.607 | −9.644 | −5.477 | 1.00 | 14.61 | | S |
| ANISOU | 1951 | SG | CYS | B | 83 | 1067 | 2633 | 1850 | −424 | 63 | −501 | S |
| ATOM | 1952 | N | LEU | B | 84 | 0.609 | −8.235 | −6.898 | 1.00 | 9.53 | | N |
| ANISOU | 1952 | N | LEU | B | 84 | 1184 | 1560 | 876 | −311 | 105 | −507 | N |
| ATOM | 1953 | CA | LEU | B | 84 | 0.565 | −7.546 | −8.181 | 1.00 | 9.86 | | C |
| ANISOU | 1953 | CA | LEU | B | 84 | 1117 | 1613 | 1016 | −344 | 137 | −353 | C |
| ATOM | 1954 | C | LEU | B | 84 | 1.959 | −7.036 | −8.517 | 1.00 | 9.68 | | C |
| ANISOU | 1954 | C | LEU | B | 84 | 1089 | 1567 | 1022 | −378 | 65 | −444 | C |
| ATOM | 1955 | O | LEU | B | 84 | 2.455 | −6.085 | −7.956 | 1.00 | 13.97 | | O |
| ANISOU | 1955 | O | LEU | B | 84 | 1393 | 2155 | 1762 | −596 | 95 | −1124 | O |
| ATOM | 1956 | CB | LEU | B | 84 | −0.433 | −6.387 | −8.234 | 1.00 | 11.15 | | C |
| ANISOU | 1956 | CB | LEU | B | 84 | 1102 | 2025 | 1111 | −136 | −341 | −461 | C |
| ATOM | 1957 | CG | LEU | B | 84 | −1.909 | −6.831 | −8.499 | 1.00 | 15.19 | | C |
| ANISOU | 1957 | CG | LEU | B | 84 | 996 | 2316 | 2459 | −374 | 277 | −629 | C |
| ATOM | 1958 | CD1 | LEU | B | 84 | −2.305 | −7.812 | −7.466 | 1.00 | 28.84 | | C |
| ANISOU | 1958 | CD1 | LEU | B | 84 | 1278 | 4754 | 4928 | −481 | 53 | 1865 | C |
| ATOM | 1959 | CD2 | LEU | B | 84 | −2.784 | −5.623 | −8.561 | 1.00 | 22.63 | | C |
| ANISOU | 1959 | CD2 | LEU | B | 84 | 1224 | 3956 | 3419 | 806 | 432 | 219 | C |
| ATOM | 1960 | N | LEU | B | 85 | 2.603 | −7.728 | −9.443 | 1.00 | 9.14 | | N |
| ANISOU | 1960 | N | LEU | B | 85 | 1054 | 1528 | 890 | −226 | 53 | −276 | N |
| ATOM | 1961 | CA | LEU | B | 85 | 3.913 | −7.350 | −9.933 | 1.00 | 8.41 | | C |
| ANISOU | 1961 | CA | LEU | B | 85 | 800 | 1417 | 979 | −168 | −204 | −96 | C |
| ATOM | 1962 | C | LEU | B | 85 | 3.748 | −6.363 | −11.062 | 1.00 | 9.66 | | C |
| ANISOU | 1962 | C | LEU | B | 85 | 928 | 1507 | 1234 | −422 | −403 | 120 | C |
| ATOM | 1963 | O | LEU | B | 85 | 3.077 | −6.691 | −12.057 | 1.00 | 9.65 | | O |
| ANISOU | 1963 | O | LEU | B | 85 | 864 | 1663 | 1140 | −559 | −339 | 122 | O |
| ATOM | 1964 | CB | LEU | B | 85 | 4.671 | −8.590 | −10.408 | 1.00 | 10.89 | | C |
| ANISOU | 1964 | CB | LEU | B | 85 | 1016 | 1863 | 1259 | 268 | −126 | −63 | C |
| ATOM | 1965 | CG | LEU | B | 85 | 4.941 | −9.676 | −9.361 | 1.00 | 10.52 | | C |
| ANISOU | 1965 | CG | LEU | B | 85 | 1322 | 1338 | 1336 | −111 | −284 | −259 | C |
| ATOM | 1966 | CD1 | LEU | B | 85 | 5.635 | −10.827 | −10.076 | 1.00 | 22.07 | | C |
| ANISOU | 1966 | CD1 | LEU | B | 85 | 3875 | 2524 | 1986 | 1769 | 80 | −174 | C |
| ATOM | 1967 | CD2 | LEU | B | 85 | 5.836 | −9.136 | −8.281 | 1.00 | 17.36 | | C |
| ANISOU | 1967 | CD2 | LEU | B | 85 | 2043 | 2999 | 1554 | −904 | −915 | 257 | C |
| ATOM | 1968 | N | THR | B | 86 | 4.318 | −5.173 | −10.892 | 1.00 | 9.09 | | N |
| ANISOU | 1968 | N | THR | B | 86 | 1004 | 1339 | 1112 | −245 | −123 | −156 | N |
| ATOM | 1969 | CA | THR | B | 86 | 4.056 | −4.070 | −11.786 | 1.00 | 8.68 | | C |
| ANISOU | 1969 | CA | THR | B | 86 | 761 | 1282 | 1255 | −151 | −6 | −143 | C |
| ATOM | 1970 | C | THR | B | 86 | 5.312 | −3.611 | −12.491 | 1.00 | 8.58 | | C |
| ANISOU | 1970 | C | THR | B | 86 | 825 | 1315 | 1121 | −457 | −62 | −271 | C |
| ATOM | 1971 | O | THR | B | 86 | 6.345 | −3.484 | −11.823 | 1.00 | 10.07 | | O |
| ANISOU | 1971 | O | THR | B | 86 | 868 | 1664 | 1295 | −356 | −234 | −189 | O |
| ATOM | 1972 | CB | THR | B | 86 | 3.495 | −2.881 | −10.989 | 1.00 | 10.54 | | C |
| ANISOU | 1972 | CB | THR | B | 86 | 1069 | 1481 | 1453 | −104 | 85 | −428 | C |
| ATOM | 1973 | OG1 | THR | B | 86 | 2.375 | −3.332 | −10.218 | 1.00 | 13.66 | | O |
| ANISOU | 1973 | OG1 | THR | B | 86 | 1505 | 1932 | 1752 | −332 | 514 | −732 | O |
| ATOM | 1974 | CG2 | THR | B | 86 | 2.975 | −1.762 | −11.889 | 1.00 | 12.23 | | C |
| ANISOU | 1974 | CG2 | THR | B | 86 | 1264 | 1326 | 2059 | 34 | −159 | −517 | C |
| ATOM | 1975 | N | PHE | B | 87 | 5.223 | −3.357 | −13.789 | 1.00 | 8.93 | | N |
| ANISOU | 1975 | N | PHE | B | 87 | 607 | 1593 | 1193 | −129 | −46 | −138 | N |
| ATOM | 1976 | CA | PHE | B | 87 | 6.298 | −2.740 | −14.544 | 1.00 | 8.41 | | C |
| ANISOU | 1976 | CA | PHE | B | 87 | 891 | 1243 | 1061 | −242 | −137 | −40 | C |
| ATOM | 1977 | C | PHE | B | 87 | 5.672 | −1.626 | −15.366 | 1.00 | 9.18 | | C |
| ANISOU | 1977 | C | PHE | B | 87 | 871 | 1057 | 1560 | −319 | −388 | −157 | C |
| ATOM | 1978 | O | PHE | B | 87 | 4.454 | −1.525 | −15.385 | 1.00 | 10.77 | | O |
| ANISOU | 1978 | O | PHE | B | 87 | 916 | 1744 | 1434 | −36 | −300 | −4 | O |
| ATOM | 1979 | CB | PHE | B | 87 | 7.055 | −3.764 | −15.394 | 1.00 | 8.65 | | C |
| ANISOU | 1979 | CB | PHE | B | 87 | 499 | 1532 | 1255 | −137 | −120 | −137 | C |
| ATOM | 1980 | CG | PHE | B | 87 | 6.312 | −4.326 | −16.577 | 1.00 | 9.43 | | C |
| ANISOU | 1980 | CG | PHE | B | 87 | 1044 | 1454 | 1085 | −298 | −194 | −99 | C |
| ATOM | 1981 | CD1 | PHE | B | 87 | 5.420 | −5.388 | −16.366 | 1.00 | 9.01 | | C |
| ANISOU | 1981 | CD1 | PHE | B | 87 | 933 | 1423 | 1067 | −215 | 31 | −289 | C |
| ATOM | 1982 | CD2 | PHE | B | 87 | 6.500 | −3.813 | −17.844 | 1.00 | 9.88 | | C |
| ANISOU | 1982 | CD2 | PHE | B | 87 | 1171 | 1372 | 1212 | −97 | −42 | −51 | C |
| ATOM | 1983 | CE1 | PHE | B | 87 | 4.739 | −5.897 | −17.457 | 1.00 | 9.78 | | C |
| ANISOU | 1983 | CE1 | PHE | B | 87 | 843 | 1706 | 1167 | −405 | 14 | −241 | C |
| ATOM | 1984 | CE2 | PHE | B | 87 | 5.808 | −4.299 | −18.919 | 1.00 | 9.32 | | C |
| ANISOU | 1984 | CE2 | PHE | B | 87 | 1093 | 1548 | 900 | 83 | 106 | −31 | C |
| ATOM | 1985 | CZ | PHE | B | 87 | 4.921 | −5.336 | −18.711 | 1.00 | 9.62 | | C |
| ANISOU | 1985 | CZ | PHE | B | 87 | 894 | 1669 | 1093 | −5 | −101 | −210 | C |
| ATOM | 1986 | N | SER | B | 88 | 6.506 | −0.832 | −16.014 | 1.00 | 9.19 | | N |
| ANISOU | 1986 | N | SER | B | 88 | 1064 | 1184 | 1243 | −31 | −164 | 33 | N |
| ATOM | 1987 | CA | SER | B | 88 | 6.033 | 0.176 | −16.931 | 1.00 | 11.15 | | C |
| ANISOU | 1987 | CA | SER | B | 88 | 1308 | 1635 | 1294 | 182 | −217 | 151 | C |
| ATOM | 1988 | C | SER | B | 88 | 6.434 | −0.214 | −18.351 | 1.00 | 10.12 | | C |
| ANISOU | 1988 | C | SER | B | 88 | 1212 | 1352 | 1280 | −274 | −127 | 59 | C |
| ATOM | 1989 | O | SER | B | 88 | 7.585 | −0.609 | −18.587 | 1.00 | 11.21 | | O |
| ANISOU | 1989 | O | SER | B | 88 | 1198 | 1552 | 1509 | −312 | 35 | 104 | O |
| ATOM | 1990 | CB | SER | B | 88 | 6.600 | 1.570 | −16.542 | 1.00 | 18.80 | | C |
| ANISOU | 1990 | CB | SER | B | 88 | 4542 | 1055 | 1546 | 174 | 74 | −31 | C |
| ATOM | 1991 | OG | SER | B | 88 | 6.134 | 2.523 | −17.495 | 1.00 | 36.72 | | O |
| ANISOU | 1991 | OG | SER | B | 88 | 7358 | 2092 | 4503 | −507 | −2622 | 1220 | O |
| ATOM | 1992 | N | VAL | B | 89 | 5.539 | −0.107 | −19.336 | 1.00 | 11.59 | | N |
| ANISOU | 1992 | N | VAL | B | 89 | 1355 | 1728 | 1323 | −428 | −166 | 232 | N |
| ATOM | 1993 | CA | VAL | B | 89 | 5.821 | −0.514 | −20.707 | 1.00 | 11.59 | | C |
| ANISOU | 1993 | CA | VAL | B | 89 | 1163 | 2067 | 1172 | −252 | −142 | 433 | C |
| ATOM | 1994 | C | VAL | B | 89 | 6.849 | 0.358 | −21.421 | 1.00 | 13.39 | | C |
| ANISOU | 1994 | C | VAL | B | 89 | 1221 | 2242 | 1625 | −344 | 62 | 310 | C |
| ATOM | 1995 | O | VAL | B | 89 | 7.402 | −0.055 | −22.416 | 1.00 | 15.10 | | O |
| ANISOU | 1995 | O | VAL | B | 89 | 1682 | 2445 | 1612 | −439 | 312 | 331 | O |
| ATOM | 1996 | CB | VAL | B | 89 | 4.545 | −0.634 | −21.576 | 1.00 | 12.47 | | C |
| ANISOU | 1996 | CB | VAL | B | 89 | 1321 | 1851 | 1565 | −177 | −353 | 46 | C |
| ATOM | 1997 | CG1 | VAL | B | 89 | 3.607 | −1.676 | −20.970 | 1.00 | 12.65 | | C |
| ANISOU | 1997 | CG1 | VAL | B | 89 | 1320 | 1798 | 1687 | −333 | 19 | −592 | C |
| ATOM | 1998 | CG2 | VAL | B | 89 | 3.837 | 0.696 | −21.768 | 1.00 | 12.95 | | C |
| ANISOU | 1998 | CG2 | VAL | B | 89 | 1458 | 2069 | 1393 | 89 | −388 | −100 | C |
| ATOM | 1999 | N | ASP | B | 90 | 7.153 | 1.524 | −20.888 | 1.00 | 14.58 | | N |
| ANISOU | 1999 | N | ASP | B | 90 | 1681 | 2217 | 1642 | −474 | 138 | 393 | N |
| ATOM | 2000 | CA | ASP | B | 90 | 8.244 | 2.347 | −21.362 | 1.00 | 16.69 | | C |
| ANISOU | 2000 | CA | ASP | B | 90 | 1959 | 2036 | 2345 | −528 | −19 | 887 | C |
| ATOM | 2001 | C | ASP | B | 90 | 9.551 | 2.119 | −20.600 | 1.00 | 15.41 | | C |
| ANISOU | 2001 | C | ASP | B | 90 | 1521 | 1901 | 2435 | −255 | 355 | 521 | C |
| ATOM | 2002 | O | ASP | B | 90 | 10.473 | 2.927 | −20.803 | 1.00 | 21.32 | | O |
| ANISOU | 2002 | O | ASP | B | 90 | 2055 | 2455 | 3590 | −846 | −108 | 903 | O |
| ATOM | 2003 | CB | ASP | B | 90 | 7.933 | 3.827 | −21.270 | 1.00 | 18.22 | | C |
| ANISOU | 2003 | CB | ASP | B | 90 | 1845 | 2155 | 2924 | −297 | −496 | 675 | C |
| ATOM | 2004 | CG | ASP | B | 90 | 7.928 | 4.373 | −19.851 | 1.00 | 20.54 | | C |
| ANISOU | 2004 | CG | ASP | B | 90 | 1968 | 2724 | 3111 | −31 | 321 | 301 | C |
| ATOM | 2005 | OD1 | ASP | B | 90 | 7.666 | 3.631 | −18.887 | 1.00 | 28.34 | | O |
| ANISOU | 2005 | OD1 | ASP | B | 90 | 4418 | 3294 | 3054 | 709 | 363 | 793 | O |
| ATOM | 2006 | OD2 | ASP | B | 90 | 8.168 | 5.607 | −19.717 | 1.00 | 26.41 | | O |
| ANISOU | 2006 | OD2 | ASP | B | 90 | 3351 | 2883 | 3801 | −331 | −165 | −138 | O |
| ATOM | 2007 | N | ASP | B | 91 | 9.628 | 1.069 | −19.770 | 1.00 | 15.94 | | N |
| ANISOU | 2007 | N | ASP | B | 91 | 1475 | 2047 | 2535 | −128 | 104 | 618 | N |
| ATOM | 2008 | CA | ASP | B | 91 | 10.846 | 0.827 | −18.990 | 1.00 | 15.93 | | C |
| ANISOU | 2008 | CA | ASP | B | 91 | 1069 | 2295 | 2691 | −460 | 228 | 681 | C |
| ATOM | 2009 | C | ASP | B | 91 | 11.200 | −0.650 | −19.058 | 1.00 | 14.58 | | C |
| ANISOU | 2009 | C | ASP | B | 91 | 1124 | 2414 | 2001 | −225 | −104 | 586 | C |
| ATOM | 2010 | O | ASP | B | 91 | 10.708 | −1.414 | −18.253 | 1.00 | 13.81 | | O |
| ANISOU | 2010 | O | ASP | B | 91 | 1202 | 2099 | 1947 | −433 | −5 | 252 | O |
| ATOM | 2011 | CB | ASP | B | 91 | 10.648 | 1.302 | −17.568 | 1.00 | 19.80 | | C |
| ANISOU | 2011 | CB | ASP | B | 91 | 2146 | 2417 | 2960 | −299 | −324 | 59 | C |
| ATOM | 2012 | CG | ASP | B | 91 | 11.713 | 1.112 | −16.528 | 1.00 | 22.52 | | C |
| ANISOU | 2012 | CG | ASP | B | 91 | 2175 | 2697 | 3687 | −933 | −926 | −822 | C |
| ATOM | 2013 | OD1 | ASP | B | 91 | 12.715 | 0.476 | −16.866 | 1.00 | 20.93 | | O |
| ANISOU | 2013 | OD1 | ASP | B | 91 | 2034 | 2710 | 3208 | −823 | −793 | 139 | O |
| ATOM | 2014 | OD2 | ASP | B | 91 | 11.535 | 1.567 | −15.373 | 1.00 | 27.94 | | O |
| ANISOU | 2014 | OD2 | ASP | B | 91 | 3006 | 3881 | 3730 | −734 | −955 | −1139 | O |
| ATOM | 2015 | N | SER | B | 92 | 12.032 | −1.038 | −20.002 | 1.00 | 15.06 | | N |
| ANISOU | 2015 | N | SER | B | 92 | 1091 | 2678 | 1954 | −299 | −90 | 497 | N |
| ATOM | 2016 | CA | SER | B | 92 | 12.521 | −2.386 | −20.177 | 1.00 | 16.32 | | C |
| ANISOU | 2016 | CA | SER | B | 92 | 1428 | 2914 | 1857 | 162 | −82 | 660 | C |
| ATOM | 2017 | C | SER | B | 92 | 13.104 | −2.897 | −18.878 | 1.00 | 13.56 | | C |
| ANISOU | 2017 | C | SER | B | 92 | 1085 | 2665 | 1402 | −232 | 272 | 479 | C |
| ATOM | 2018 | O | SER | B | 92 | 12.921 | −4.045 | −18.491 | 1.00 | 14.33 | | O |
| ANISOU | 2018 | O | SER | B | 92 | 1213 | 2742 | 1491 | −343 | 74 | 516 | O |
| ATOM | 2019 | CB | SER | B | 92 | 13.614 | −2.409 | −21.296 | 1.00 | 25.64 | | C |
| ANISOU | 2019 | CB | SER | B | 92 | 3443 | 5151 | 1147 | 1165 | 473 | 383 | C |
| ATOM | 2020 | OG | SER | B | 92 | 14.305 | −3.638 | −21.238 | 1.00 | 37.38 | | O |
| ANISOU | 2020 | OG | SER | B | 92 | 4344 | 5720 | 4141 | 1892 | 1159 | −605 | O |
| ATOM | 2021 | N | GLN | B | 93 | 13.891 | −2.092 | −18.181 | 1.00 | 14.52 | | N |
| ANISOU | 2021 | N | GLN | B | 93 | 826 | 2586 | 2105 | −90 | 92 | 383 | N |
| ATOM | 2022 | CA | GLN | B | 93 | 14.504 | −2.574 | −16.964 | 1.00 | 14.37 | | C |
| ANISOU | 2022 | CA | GLN | B | 93 | 1101 | 2333 | 2025 | −247 | −90 | 99 | C |
| ATOM | 2023 | C | GLN | B | 93 | 13.469 | −2.993 | −15.940 | 1.00 | 13.28 | | C |
| ANISOU | 2023 | C | GLN | B | 93 | 1375 | 1967 | 1705 | −221 | −141 | −122 | C |
| ATOM | 2024 | O | GLN | B | 93 | 13.624 | −4.027 | −15.281 | 1.00 | 12.54 | | O |
| ANISOU | 2024 | O | GLN | B | 93 | 985 | 2173 | 1607 | −419 | −530 | 8 | O |
| ATOM | 2025 | CB | GLN | B | 93 | 15.427 | −1.496 | −16.375 | 1.00 | 19.69 | | C |
| ANISOU | 2025 | CB | GLN | B | 93 | 2090 | 2812 | 2581 | −979 | −261 | 59 | C |
| ATOM | 2026 | CG | GLN | B | 93 | 16.227 | −2.038 | −15.218 | 1.00 | 24.42 | | C |
| ANISOU | 2026 | CG | GLN | B | 93 | 1906 | 3895 | 3477 | −1250 | −1014 | 204 | C |
| ATOM | 2027 | CD | GLN | B | 93 | 17.026 | −3.271 | −15.601 | 1.00 | 26.17 | | C |
| ANISOU | 2027 | CD | GLN | B | 93 | 1577 | 4122 | 4245 | −900 | −838 | 680 | C |
| ATOM | 2028 | OE1 | GLN | B | 93 | 18.105 | −3.107 | −16.203 | 1.00 | 32.28 | | O |
| ANISOU | 2028 | OE1 | GLN | B | 93 | 2961 | 5405 | 3899 | −453 | 341 | 1331 | O |
| ATOM | 2029 | NE2 | GLN | B | 93 | 16.537 | −4.462 | −15.273 | 1.00 | 27.78 | | N |
| ANISOU | 2029 | NE2 | GLN | B | 93 | 2953 | 3855 | 3748 | −1529 | −780 | −368 | N |
| ATOM | 2030 | N | SER | B | 94 | 12.392 | −2.230 | −15.762 | 1.00 | 11.85 | | N |
| ANISOU | 2030 | N | SER | B | 94 | 1106 | 1769 | 1628 | −468 | −172 | −343 | N |
| ATOM | 2031 | CA | SER | B | 94 | 11.352 | −2.640 | −14.816 | 1.00 | 11.37 | | C |
| ANISOU | 2031 | CA | SER | B | 94 | 1369 | 1696 | 1255 | −319 | −279 | 25 | C |
| ATOM | 2032 | C | SER | B | 94 | 10.701 | −3.964 | −15.190 | 1.00 | 9.92 | | C |
| ANISOU | 2032 | C | SER | B | 94 | 766 | 1806 | 1195 | −222 | 9 | −174 | C |
| ATOM | 2033 | O | SER | B | 94 | 10.325 | −4.731 | −14.269 | 1.00 | 10.00 | | O |
| ANISOU | 2033 | O | SER | B | 94 | 869 | 1866 | 1064 | −386 | −130 | −238 | O |
| ATOM | 2034 | CB | SER | B | 94 | 10.292 | −1.561 | −14.666 | 1.00 | 11.27 | | C |
| ANISOU | 2034 | CB | SER | B | 94 | 1211 | 1774 | 1296 | −372 | −112 | −220 | C |
| ATOM | 2035 | OG | SER | B | 94 | 9.414 | −1.411 | −15.793 | 1.00 | 11.51 | | O |
| ANISOU | 2035 | OG | SER | B | 94 | 1214 | 1672 | 1487 | −382 | −213 | 0 | O |
| ATOM | 2036 | N | PHE | B | 95 | 10.580 | −4.227 | −16.484 | 1.00 | 10.61 | | N |
| ANISOU | 2036 | N | PHE | B | 95 | 1016 | 1834 | 1182 | −245 | −105 | −43 | N |
| ATOM | 2037 | CA | PHE | B | 95 | 10.052 | −5.511 | −16.917 | 1.00 | 9.47 | | C |
| ANISOU | 2037 | CA | PHE | B | 95 | 905 | 1789 | 905 | −95 | −35 | −58 | C |
| ATOM | 2038 | C | PHE | B | 95 | 11.025 | −6.638 | −16.631 | 1.00 | 10.49 | | C |
| ANISOU | 2038 | C | PHE | B | 95 | 845 | 1949 | 1192 | −19 | −39 | −66 | C |
| ATOM | 2039 | O | PHE | B | 95 | 10.649 | −7.713 | −16.145 | 1.00 | 9.65 | | O |
| ANISOU | 2039 | O | PHE | B | 95 | 884 | 1831 | 950 | −44 | −184 | −65 | O |
| ATOM | 2040 | CB | PHE | B | 95 | 9.746 | −5.437 | −18.389 | 1.00 | 9.91 | | C |
| ANISOU | 2040 | CB | PHE | B | 95 | 1172 | 1685 | 910 | −402 | 38 | 129 | C |
| ATOM | 2041 | CG | PHE | B | 95 | 9.288 | −6.710 | −19.058 | 1.00 | 9.48 | | C |
| ANISOU | 2041 | CG | PHE | B | 95 | 786 | 1723 | 1093 | 33 | −396 | −64 | C |
| ATOM | 2042 | CD1 | PHE | B | 95 | 8.046 | −7.225 | −18.690 | 1.00 | 9.55 | | C |
| ANISOU | 2042 | CD1 | PHE | B | 95 | 931 | 1537 | 1159 | −98 | −432 | 4 | C |
| ATOM | 2043 | CD2 | PHE | B | 95 | 10.104 | −7.317 | −19.981 | 1.00 | 10.60 | | C |
| ANISOU | 2043 | CD2 | PHE | B | 95 | 1188 | 1740 | 1098 | 84 | −266 | 13 | C |
| ATOM | 2044 | CE1 | PHE | B | 95 | 7.625 | −8.386 | −19.275 | 1.00 | 10.54 | | C |
| ANISOU | 2044 | CE1 | PHE | B | 95 | 1132 | 1756 | 1116 | −158 | −120 | −230 | C |
| ATOM | 2045 | CE2 | PHE | B | 95 | 9.656 | −8.455 | −20.614 | 1.00 | 11.37 | | C |
| ANISOU | 2045 | CE2 | PHE | B | 95 | 862 | 2164 | 1294 | 116 | −243 | −430 | C |
| ATOM | 2046 | CZ2 | PHE | B | 95 | 8.426 | −8.944 | −20.263 | 1.00 | 10.83 | | C |
| ANISOU | 2046 | CZ2 | PHE | B | 95 | 859 | 1893 | 1362 | 186 | −240 | −250 | C |
| ATOM | 2047 | N | GLN | B | 96 | 12.299 | −6.431 | −16.922 | 1.00 | 10.19 | | N |
| ANISOU | 2047 | N | GLN | B | 96 | 830 | 2115 | 928 | −117 | −99 | −198 | N |
| ATOM | 2048 | CA | GLN | B | 96 | 13.319 | −7.432 | −16.680 | 1.00 | 11.41 | | C |
| ANISOU | 2048 | CA | GLN | B | 96 | 931 | 2320 | 1082 | 185 | 28 | −456 | C |
| ATOM | 2049 | C | GLN | B | 96 | 13.410 | −7.802 | −15.204 | 1.00 | 10.10 | | C |
| ANISOU | 2049 | C | GLN | B | 96 | 853 | 1849 | 1134 | 126 | −128 | −339 | C |
| ATOM | 2050 | O | GLN | B | 96 | 13.716 | −8.938 | −14.858 | 1.00 | 12.39 | | O |
| ANISOU | 2050 | O | GLN | B | 96 | 1238 | 1968 | 1502 | 538 | −433 | −534 | O |
| ATOM | 2051 | CB | GLN | B | 96 | 14.690 | −6.941 | −17.143 | 1.00 | 13.01 | | C |
| ANISOU | 2051 | CB | GLN | B | 96 | 903 | 2499 | 1541 | 58 | −35 | 132 | C |
| ATOM | 2052 | CG | GLN | B | 96 | 14.852 | −6.761 | −18.647 | 1.00 | 14.79 | | C |
| ANISOU | 2052 | CG | GLN | B | 96 | 1576 | 2355 | 1690 | 232 | 389 | 169 | C |
| ATOM | 2053 | CD | GLN | B | 96 | 14.652 | −8.128 | −19.265 | 1.00 | 17.98 | | C |
| ANISOU | 2053 | CD | GLN | B | 96 | 1897 | 2847 | 2090 | 458 | 263 | −500 | C |
| ATOM | 2054 | OE1 | GLN | B | 96 | 15.547 | −8.994 | −19.218 | 1.00 | 25.08 | | O |
| ANISOU | 2054 | OE1 | GLN | B | 96 | 3843 | 2928 | 2760 | 1426 | −999 | −417 | O |
| ATOM | 2055 | NE2 | GLN | B | 96 | 13.449 | −8.305 | −19.722 | 1.00 | 30.77 | | N |
| ANISOU | 2055 | NE2 | GLN | B | 96 | 2438 | 4210 | 5044 | 440 | −854 | −1925 | N |
| ATOM | 2056 | N | ASN | B | 97 | 13.092 | −6.833 | −14.346 | 1.00 | 8.64 | | N |
| ANISOU | 2056 | N | ASN | B | 97 | 899 | 1463 | 922 | −77 | −168 | −72 | N |
| ATOM | 2057 | CA | ASN | B | 97 | 13.082 | −7.130 | −12.919 | 1.00 | 9.41 | | C |
| ANISOU | 2057 | CA | ASN | B | 97 | 895 | 1751 | 929 | −233 | −185 | −79 | C |
| ATOM | 2058 | C | ASN | B | 97 | 11.944 | −7.876 | −12.333 | 1.00 | 9.76 | | C |
| ANISOU | 2058 | C | ASN | B | 97 | 870 | 1814 | 1024 | −252 | −21 | −123 | C |
| ATOM | 2059 | O | ASN | B | 97 | 11.990 | −8.123 | −11.129 | 1.00 | 9.91 | | O |
| ANISOU | 2059 | O | ASN | B | 97 | 862 | 1917 | 988 | −112 | 52 | −201 | O |
| ATOM | 2060 | CB | ASN | B | 97 | 13.289 | −5.768 | −12.171 | 1.00 | 10.78 | | C |
| ANISOU | 2060 | CB | ASN | B | 97 | 814 | 2154 | 1129 | −395 | −155 | −422 | C |
| ATOM | 2061 | CG | ASN | B | 97 | 14.689 | −5.177 | −12.288 | 1.00 | 12.21 | | C |
| ANISOU | 2061 | CG | ASN | B | 97 | 796 | 1980 | 1863 | −345 | −167 | −417 | C |
| ATOM | 2062 | OD1 | ASN | B | 97 | 14.899 | −3.935 | −12.195 | 1.00 | 15.16 | | O |
| ANISOU | 2062 | OD1 | ASN | B | 97 | 1362 | 1901 | 2496 | −407 | −186 | −417 | O |
| ATOM | 2063 | ND2 | ASN | B | 97 | 15.734 | −5.945 | −12.463 | 1.00 | 13.13 | | N |
| ANISOU | 2063 | ND2 | ASN | B | 97 | 815 | 2095 | 2078 | −217 | −256 | −484 | N |
| ATOM | 2064 | N | LEU | B | 98 | 10.952 | −8.256 | −13.139 | 1.00 | 8.78 | | N |
| ANISOU | 2064 | N | LEU | B | 98 | 796 | 1487 | 1051 | −54 | 6 | −356 | N |
| ATOM | 2065 | CA | LEU | B | 98 | 9.815 | −8.933 | −12.529 | 1.00 | 8.58 | | C |
| ANISOU | 2065 | CA | LEU | B | 98 | 738 | 1328 | 1195 | −49 | −74 | −301 | C |
| ATOM | 2066 | C | LEU | B | 98 | 10.206 | −10.232 | −11.830 | 1.00 | 8.68 | | C |
| ANISOU | 2066 | C | LEU | B | 98 | 872 | 1576 | 851 | 88 | −173 | −224 | C |
| ATOM | 2067 | O | LEU | B | 98 | 9.690 | −10.534 | −10.758 | 1.00 | 9.73 | | O |
| ANISOU | 2067 | O | LEU | B | 98 | 710 | 1969 | 1019 | −24 | −250 | −65 | O |
| ATOM | 2068 | CB | LEU | B | 98 | 8.770 | −9.291 | −13.598 | 1.00 | 10.66 | | C |
| ANISOU | 2068 | CB | LEU | B | 98 | 664 | 2071 | 1317 | 35 | −296 | 27 | C |
| ATOM | 2069 | CG | LEU | B | 98 | 8.018 | −8.108 | −14.183 | 1.00 | 10.13 | | C |
| ANISOU | 2069 | CG | LEU | B | 98 | 1074 | 1642 | 1132 | −85 | −152 | −24 | C |
| ATOM | 2070 | CD1 | LEU | B | 98 | 7.126 | −8.591 | −15.324 | 1.00 | 12.24 | | C |
| ANISOU | 2070 | CD1 | LEU | B | 98 | 1304 | 2033 | 1312 | 169 | −532 | −91 | C |
| ATOM | 2071 | CD2 | LEU | B | 98 | 7.199 | −7.394 | −13.131 | 1.00 | 13.25 | | C |
| ANISOU | 2071 | CD2 | LEU | B | 98 | 1564 | 2376 | 1096 | 468 | −66 | −29 | C |
| ATOM | 2072 | N | SER | B | 99 | 11.084 | −11.028 | −12.458 | 1.00 | 9.39 | | N |
| ANISOU | 2072 | N | SER | B | 99 | 819 | 1601 | 1148 | 103 | −214 | −189 | N |
| ATOM | 2073 | CA | SER | B | 99 | 11.456 | −12.295 | −11.823 | 1.00 | 10.21 | | C |
| ANISOU | 2073 | CA | SER | B | 99 | 846 | 1534 | 1499 | 52 | −366 | −131 | C |
| ATOM | 2074 | C | SER | B | 99 | 12.050 | −12.070 | −10.451 | 1.00 | 9.17 | | C |
| ANISOU | 2074 | C | SER | B | 99 | 867 | 1442 | 1174 | 84 | −59 | −99 | C |
| ATOM | 2075 | O | SER | B | 99 | 11.773 | −12.852 | −9.529 | 1.00 | 11.01 | | O |
| ANISOU | 2075 | O | SER | B | 99 | 945 | 1729 | 1510 | 8 | −191 | 143 | O |
| ATOM | 2076 | CB | SER | B | 99 | 12.471 | −13.052 | −12.726 | 1.00 | 13.80 | | C |
| ANISOU | 2076 | CB | SER | B | 99 | 1561 | 1844 | 1840 | 168 | −232 | −972 | C |
| ATOM | 2077 | OG | SER | B | 99 | 11.955 | −13.237 | −14.024 | 1.00 | 41.90 | | O |
| ANISOU | 2077 | OG | SER | B | 99 | 5203 | 8528 | 2190 | 1280 | −970 | −2555 | O |
| ATOM | 2078 | N | ASN | B | 100 | 12.930 | −11.104 | −10.307 | 1.00 | 9.20 | | N |
| ANISOU | 2078 | N | ASN | B | 100 | 764 | 1755 | 976 | 17 | −41 | −147 | N |
| ATOM | 2079 | CA | ASN | B | 100 | 13.545 | −10.843 | −9.002 | 1.00 | 10.29 | | C |
| ANISOU | 2079 | CA | ASN | B | 100 | 1001 | 1841 | 1067 | 58 | −225 | −38 | C |
| ATOM | 2080 | C | ASN | B | 100 | 12.558 | −10.253 | −8.016 | 1.00 | 9.06 | | C |
| ANISOU | 2080 | C | ASN | B | 100 | 1032 | 1277 | 1134 | −27 | −319 | −235 | C |
| ATOM | 2081 | O | ASN | B | 100 | 12.686 | −10.529 | −6.822 | 1.00 | 9.75 | | O |
| ANISOU | 2081 | O | ASN | B | 100 | 779 | 1813 | 1112 | 19 | −234 | −124 | O |
| ATOM | 2082 | CB | ASN | B | 100 | 14.782 | −9.976 | −9.152 | 1.00 | 13.15 | | C |
| ANISOU | 2082 | CB | ASN | B | 100 | 1017 | 2600 | 1378 | −275 | −360 | −142 | C |
| ATOM | 2083 | CG | ASN | B | 100 | 15.965 | −10.747 | −9.760 | 1.00 | 14.79 | | C |
| ANISOU | 2083 | CG | ASN | B | 100 | 1028 | 3316 | 1276 | −268 | −172 | −429 | C |
| ATOM | 2084 | OD1 | ASN | B | 100 | 15.962 | −11.971 | −9.692 | 1.00 | 20.53 | | O |
| ANISOU | 2084 | OD1 | ASN | B | 100 | 1049 | 3307 | 3446 | 35 | −119 | −774 | O |
| ATOM | 2085 | ND2 | ASN | B | 100 | 16.915 | −9.994 | −10.268 | 1.00 | 21.10 | | N |
| ANISOU | 2085 | ND2 | ASN | B | 100 | 1580 | 4624 | 1813 | −630 | 139 | 228 | N |
| ATOM | 2086 | N | TRP | B | 101 | 11.557 | −9.490 | −8.456 | 1.00 | 9.54 | | N |
| ANISOU | 2086 | N | TRP | B | 101 | 1144 | 1468 | 1010 | 92 | −223 | −45 | N |
| ATOM | 2087 | CA | TRP | B | 101 | 10.502 | −9.102 | −7.506 | 1.00 | 9.47 | | C |
| ANISOU | 2087 | CA | TRP | B | 101 | 1250 | 1154 | 1193 | 120 | −120 | 124 | C |
| ATOM | 2088 | C | TRP | B | 101 | 9.734 | −10.307 | −6.992 | 1.00 | 8.72 | | C |
| ANISOU | 2088 | C | TRP | B | 101 | 994 | 1271 | 1050 | 204 | −170 | 5 | C |
| ATOM | 2089 | O | TRP | B | 101 | 9.446 | −10.363 | −5.798 | 1.00 | 8.96 | | O |
| ANISOU | 2089 | O | TRP | B | 101 | 1021 | 1275 | 1109 | −65 | −51 | −40 | O |
| ATOM | 2090 | CB | TRP | B | 101 | 9.576 | −8.089 | −8.177 | 1.00 | 10.62 | | C |
| ANISOU | 2090 | CB | TRP | B | 101 | 1188 | 1348 | 1497 | 158 | −165 | 153 | C |
| ATOM | 2091 | CG | TRP | B | 101 | 10.120 | −6.697 | −8.181 | 1.00 | 9.11 | | C |
| ANISOU | 2091 | CG | TRP | B | 101 | 1108 | 1278 | 1073 | 303 | −130 | 196 | C |
| ATOM | 2092 | CD1 | TRP | B | 101 | 10.490 | −5.948 | −9.250 | 1.00 | 10.78 | | C |
| ANISOU | 2092 | CD1 | TRP | B | 101 | 1344 | 1615 | 1137 | 75 | −14 | 251 | C |
| ATOM | 2093 | CD2 | TRP | B | 101 | 10.330 | −5.890 | −7.008 | 1.00 | 9.37 | | C |
| ANISOU | 2093 | CD2 | TRP | B | 101 | 835 | 1577 | 1146 | 120 | −327 | 120 | C |
| ATOM | 2094 | NE1 | TRP | B | 101 | 10.935 | −4.724 | −8.830 | 1.00 | 11.44 | | N |
| ANISOU | 2094 | NE1 | TRP | B | 101 | 1166 | 1703 | 1477 | −104 | 174 | 206 | N |
| ATOM | 2095 | CE2 | TRP | B | 101 | 10.844 | −4.654 | −7.469 | 1.00 | 10.44 | | C |
| ANISOU | 2095 | CE2 | TRP | B | 101 | 1162 | 1342 | 1461 | 335 | −137 | 84 | C |
| ATOM | 2096 | CE3 | TRP | B | 101 | 10.133 | −6.085 | −5.654 | 1.00 | 11.08 | | C |
| ANISOU | 2096 | CE3 | TRP | B | 101 | 1433 | 1644 | 1134 | 193 | −583 | 163 | C |
| ATOM | 2097 | CZ2 | TRP | B | 101 | 11.168 | −3.628 | −6.585 | 1.00 | 13.24 | | C |
| ANISOU | 2097 | CZ2 | TRP | B | 101 | 1655 | 1646 | 1731 | −136 | −355 | 56 | C |
| ATOM | 2098 | CZ3 | TRP | B | 101 | 10.456 | −5.061 | −4.774 | 1.00 | 12.14 | | C |
| ANISOU | 2098 | CZ3 | TRP | B | 101 | 1257 | 1998 | 1355 | −14 | 128 | −288 | C |
| ATOM | 2099 | CH2 | TRP | B | 101 | 10.978 | −3.818 | −5.248 | 1.00 | 13.57 | | C |
| ANISOU | 2099 | CH2 | TRP | B | 101 | 1648 | 1948 | 1559 | −167 | −557 | −39 | C |
| ATOM | 2100 | N | LYS | B | 102 | 9.435 | −11.243 | −7.889 | 1.00 | 9.07 | | N |
| ANISOU | 2100 | N | LYS | B | 102 | 836 | 1440 | 1169 | −71 | −153 | −102 | N |
| ATOM | 2101 | CA | LYS | B | 102 | 8.748 | −12.444 | −7.454 | 1.00 | 8.83 | | C |
| ANISOU | 2101 | CA | LYS | B | 102 | 700 | 1484 | 1171 | 36 | 3 | −48 | C |
| ATOM | 2102 | C | LYS | B | 102 | 9.609 | −13.205 | −6.440 | 1.00 | 9.12 | | C |
| ANISOU | 2102 | C | LYS | B | 102 | 1008 | 1284 | 1174 | 142 | −101 | −184 | C |
| ATOM | 2103 | O | LYS | B | 102 | 9.101 | −13.664 | −5.435 | 1.00 | 10.42 | | O |
| ANISOU | 2103 | O | LYS | B | 102 | 1105 | 1575 | 1278 | −215 | −271 | −12 | O |
| ATOM | 2104 | CB | LYS | B | 102 | 8.418 | −13.308 | −8.657 | 1.00 | 9.74 | | C |
| ANISOU | 2104 | CB | LYS | B | 102 | 662 | 1500 | 1537 | −29 | −331 | −172 | C |
| ATOM | 2105 | CG | LYS | B | 102 | 7.668 | −14.572 | −8.307 | 1.00 | 12.33 | | C |
| ANISOU | 2105 | CG | LYS | B | 102 | 1173 | 1566 | 1946 | −217 | −490 | 57 | C |
| ATOM | 2106 | CD | LYS | B | 102 | 7.434 | −15.394 | −9.581 | 1.00 | 16.32 | | C |
| ANISOU | 2106 | CD | LYS | B | 102 | 1759 | 1834 | 2608 | −462 | −739 | −405 | C |
| ATOM | 2107 | CE | LYS | B | 102 | 7.006 | −16.821 | −9.343 | 1.00 | 28.24 | | C |
| ANISOU | 2107 | CE | LYS | B | 102 | 4492 | 1477 | 4759 | −433 | −2662 | −22 | C |
| ATOM | 2108 | NZ | LYS | B | 102 | 6.820 | −17.549 | −10.643 | 1.00 | 34.09 | | N |
| ANISOU | 2108 | NZ | LYS | B | 102 | 2521 | 3037 | 7395 | 499 | −2810 | −2864 | N |
| ATOM | 2109 | N | LYS | B | 103 | 10.909 | −13.302 | −6.694 | 1.00 | 9.46 | | N |
| ANISOU | 2109 | N | LYS | B | 103 | 943 | 1422 | 1229 | 86 | −253 | 8 | N |
| ATOM | 2110 | CA | LYS | B | 103 | 11.797 | −14.003 | −5.773 | 1.00 | 8.96 | | C |
| ANISOU | 2110 | CA | LYS | B | 103 | 1149 | 1081 | 1173 | 126 | −206 | −50 | C |
| ATOM | 2111 | C | LYS | B | 103 | 11.846 | −13.310 | −4.407 | 1.00 | 8.93 | | C |
| ANISOU | 2111 | C | LYS | B | 103 | 858 | 1374 | 1160 | −166 | −218 | −82 | C |
| ATOM | 2112 | O | LYS | B | 103 | 11.839 | −13.945 | −3.357 | 1.00 | 9.13 | | O |
| ANISOU | 2112 | O | LYS | B | 103 | 826 | 1488 | 1155 | 55 | −90 | −74 | O |
| ATOM | 2113 | CB | LYS | B | 103 | 13.207 | −14.144 | −6.335 | 1.00 | 8.93 | | C |
| ANISOU | 2113 | CB | LYS | B | 103 | 1003 | 1192 | 1198 | 47 | −382 | −133 | C |
| ATOM | 2114 | CG | LYS | B | 103 | 13.318 | −15.125 | −7.489 | 1.00 | 10.51 | | C |
| ANISOU | 2114 | CG | LYS | B | 103 | 1127 | 1571 | 1294 | 297 | −348 | −317 | C |
| ATOM | 2115 | CD | LYS | B | 103 | 14.725 | −15.249 | −8.033 | 1.00 | 12.67 | | C |
| ANISOU | 2115 | CD | LYS | B | 103 | 1225 | 2095 | 1492 | 140 | −199 | −407 | C |
| ATOM | 2116 | CE | LYS | B | 103 | 14.827 | −16.203 | −9.196 | 1.00 | 17.36 | | C |
| ANISOU | 2116 | CE | LYS | B | 103 | 1682 | 3190 | 1723 | 899 | −304 | −965 | C |
| ATOM | 2117 | NZ | LYS | B | 103 | 16.224 | −16.484 | −9.600 | 1.00 | 18.63 | | N |
| ANISOU | 2117 | NZ | LYS | B | 103 | 1674 | 3127 | 2277 | 244 | 275 | −882 | N |
| ATOM | 2118 | N | GLU | B | 104 | 11.912 | −11.992 | −4.410 | 1.00 | 8.14 | | N |
| ANISOU | 2118 | N | GLU | B | 104 | 725 | 1374 | 994 | −32 | −135 | −163 | N |
| ATOM | 2119 | CA | GLU | B | 104 | 11.946 | −11.213 | −3.183 | 1.00 | 8.31 | | C |
| ANISOU | 2119 | CA | GLU | B | 104 | 729 | 1401 | 1028 | 43 | −171 | −129 | C |
| ATOM | 2120 | C | GLU | B | 104 | 10.682 | −11.431 | −2.366 | 1.00 | 8.52 | | C |
| ANISOU | 2120 | C | GLU | B | 104 | 721 | 1361 | 1154 | 118 | −71 | −231 | C |
| ATOM | 2121 | O | GLU | B | 104 | 10.718 | −11.661 | −1.161 | 1.00 | 9.41 | | O |
| ANISOU | 2121 | O | GLU | B | 104 | 935 | 1447 | 1194 | −67 | −36 | −105 | O |
| ATOM | 2122 | CB | GLU | B | 104 | 12.135 | −9.717 | −3.487 | 1.00 | 9.38 | | C |
| ANISOU | 2122 | CB | GLU | B | 104 | 858 | 1396 | 1310 | −37 | −125 | −245 | C |
| ATOM | 2123 | CG | GLU | B | 104 | 12.235 | −8.868 | −2.251 | 1.00 | 9.90 | | C |
| ANISOU | 2123 | CG | GLU | B | 104 | 1127 | 1425 | 1209 | 136 | −71 | −231 | C |
| ATOM | 2124 | CD | GLU | B | 104 | 12.340 | −7.370 | −2.499 | 1.00 | 9.20 | | C |
| ANISOU | 2124 | CD | GLU | B | 104 | 786 | 1385 | 1324 | 134 | −118 | −190 | C |
| ATOM | 2125 | OE1 | GLU | B | 104 | 13.144 | −6.992 | −3.377 | 1.00 | 12.14 | | O |
| ANISOU | 2125 | OE1 | GLU | B | 104 | 1236 | 1791 | 1587 | −233 | 169 | −194 | O |
| ATOM | 2126 | OE2 | GLU | B | 104 | 11.606 | −6.602 | −1.843 | 1.00 | 12.16 | | O |
| ANISOU | 2126 | OE2 | GLU | B | 104 | 1274 | 1543 | 1804 | 180 | 106 | −464 | O |
| ATOM | 2127 | N | PHE | B | 105 | 9.529 | −11.332 | −3.016 | 1.00 | 8.12 | | N |
| ANISOU | 2127 | N | PHE | B | 105 | 715 | 1129 | 1243 | 33 | −35 | −147 | N |
| ATOM | 2128 | CA | PHE | B | 105 | 8.252 | −11.583 | −2.355 | 1.00 | 8.75 | | C |
| ANISOU | 2128 | CA | PHE | B | 105 | 672 | 1239 | 1415 | 170 | −64 | −76 | C |
| ATOM | 2129 | C | PHE | B | 105 | 8.236 | −12.991 | −1.752 | 1.00 | 8.97 | | C |
| ANISOU | 2129 | C | PHE | B | 105 | 722 | 1349 | 1339 | −50 | −94 | −9 | C |
| ATOM | 2130 | O | PHE | B | 105 | 7.922 | −13.170 | −0.574 | 1.00 | 9.95 | | O |
| ANISOU | 2130 | O | PHE | B | 105 | 813 | 1661 | 1307 | 233 | −127 | 91 | O |
| ATOM | 2131 | CB | PHE | B | 105 | 7.079 | −11.405 | −3.339 | 1.00 | 9.01 | | C |
| ANISOU | 2131 | CB | PHE | B | 105 | 757 | 1254 | 1413 | −4 | −199 | −138 | C |
| ATOM | 2132 | CG | PHE | B | 105 | 5.774 | −11.732 | −2.651 | 1.00 | 8.16 | | C |
| ANISOU | 2132 | CG | PHE | B | 105 | 737 | 1395 | 967 | −9 | −324 | −11 | C |
| ATOM | 2133 | CD1 | PHE | B | 105 | 5.240 | −10.860 | −1.722 | 1.00 | 9.47 | | C |
| ANISOU | 2133 | CD1 | PHE | B | 105 | 866 | 1096 | 1637 | 151 | −39 | 90 | C |
| ATOM | 2134 | CD2 | PHE | B | 105 | 5.115 | −12.919 | −2.902 | 1.00 | 9.93 | | C |
| ANISOU | 2134 | CD2 | PHE | B | 105 | 809 | 1626 | 1338 | −199 | −401 | −200 | C |
| ATOM | 2135 | CE1 | PHE | B | 105 | 4.076 | −11.187 | −1.048 | 1.00 | 10.72 | | C |
| ANISOU | 2135 | CE1 | PHE | B | 105 | 925 | 1740 | 1410 | −93 | −43 | −94 | C |
| ATOM | 2136 | CE2 | PHE | B | 105 | 3.935 | −13.220 | −2.241 | 1.00 | 11.24 | | C |
| ANISOU | 2136 | CE2 | PHE | B | 105 | 894 | 1566 | 1812 | −194 | −330 | 38 | C |
| ATOM | 2137 | CZ | PHE | B | 105 | 3.441 | −12.341 | −1.307 | 1.00 | 10.18 | | C |
| ANISOU | 2137 | CZ | PHE | B | 105 | 698 | 1678 | 1493 | −88 | −258 | 251 | C |
| ATOM | 2138 | N | ILE | B | 106 | 8.569 | −14.018 | −2.545 | 1.00 | 8.60 | | N |
| ANISOU | 2138 | N | ILE | B | 106 | 754 | 1192 | 1322 | 44 | −123 | 109 | N |
| ATOM | 2139 | CA | ILE | B | 106 | 8.443 | −15.392 | −2.015 | 1.00 | 9.61 | | C |
| ANISOU | 2139 | CA | ILE | B | 106 | 955 | 1307 | 1388 | −112 | −227 | 180 | C |
| ATOM | 2140 | C | ILE | B | 106 | 9.380 | −15.576 | −0.842 | 1.00 | 9.88 | | C |
| ANISOU | 2140 | C | ILE | B | 106 | 1029 | 1356 | 1369 | 78 | −188 | 216 | C |
| ATOM | 2141 | O | ILE | B | 106 | 9.009 | −16.231 | 0.130 | 1.00 | 11.66 | | O |
| ANISOU | 2141 | O | ILE | B | 106 | 1510 | 1591 | 1327 | −135 | −202 | 309 | O |
| ATOM | 2142 | CB | ILE | B | 106 | 8.698 | −16.381 | −3.151 | 1.00 | 11.01 | | C |
| ANISOU | 2142 | CB | ILE | B | 106 | 1228 | 1241 | 1714 | 64 | −457 | −11 | C |
| ATOM | 2143 | CG1 | ILE | B | 106 | 7.550 | −16.367 | −4.171 | 1.00 | 13.34 | | C |
| ANISOU | 2143 | CG1 | ILE | B | 106 | 1413 | 1710 | 1944 | −128 | −691 | −249 | C |
| ATOM | 2144 | CG2 | ILE | B | 106 | 9.002 | −17.781 | −2.630 | 1.00 | 12.96 | | C |
| ANISOU | 2144 | CG2 | ILE | B | 106 | 1865 | 1228 | 1832 | −232 | −360 | 137 | C |
| ATOM | 2145 | CD1 | ILE | B | 106 | 7.799 | −17.142 | −5.455 | 1.00 | 21.83 | | C |
| ANISOU | 2145 | CD1 | ILE | B | 106 | 2644 | 3519 | 2132 | −114 | −645 | −936 | C |
| ATOM | 2146 | N | TYR | B | 107 | 10.601 | −15.034 | −0.948 | 1.00 | 9.90 | | N |
| ANISOU | 2146 | N | TYR | B | 107 | 1031 | 1609 | 1120 | 38 | −221 | 187 | N |
| ATOM | 2147 | CA | TYR | B | 107 | 11.550 | −15.235 | 0.149 | 1.00 | 9.55 | | C |
| ANISOU | 2147 | CA | TYR | B | 107 | 1179 | 1329 | 1121 | 39 | −305 | 98 | C |
| ATOM | 2148 | C | TYR | B | 107 | 11.104 | −14.565 | 1.439 | 1.00 | 10.22 | | C |
| ANISOU | 2148 | C | TYR | B | 107 | 1145 | 1575 | 1161 | 17 | −176 | 26 | C |
| ATOM | 2149 | O | TYR | B | 107 | 11.134 | −15.182 | 2.500 | 1.00 | 11.87 | | O |
| ANISOU | 2149 | O | TYR | B | 107 | 1566 | 1726 | 1220 | 256 | −30 | 136 | O |
| ATOM | 2150 | CB | TYR | B | 107 | 12.958 | −14.754 | −0.261 | 1.00 | 10.09 | | C |
| ANISOU | 2150 | CB | TYR | B | 107 | 917 | 1535 | 1382 | 342 | −222 | −242 | C |
| ATOM | 2151 | CG | TYR | B | 107 | 13.926 | −15.018 | 0.888 | 1.00 | 10.15 | | C |
| ANISOU | 2151 | CG | TYR | B | 107 | 1006 | 1594 | 1258 | 317 | −171 | −231 | C |
| ATOM | 2152 | CD1 | TYR | B | 107 | 14.275 | −16.298 | 1.252 | 1.00 | 13.06 | | C |
| ANISOU | 2152 | CD1 | TYR | B | 107 | 1417 | 1614 | 1932 | 417 | −787 | −284 | C |
| ATOM | 2153 | CD2 | TYR | B | 107 | 14.419 | −13.974 | 1.669 | 1.00 | 11.31 | | C |
| ANISOU | 2153 | CD2 | TYR | B | 107 | 1335 | 1542 | 1420 | 92 | −424 | −15 | C |
| ATOM | 2154 | CE1 | TYR | B | 107 | 15.113 | −16.580 | 2.300 | 1.00 | 14.24 | | C |
| ANISOU | 2154 | CE1 | TYR | B | 107 | 2186 | 1786 | 1437 | 416 | −721 | −171 | C |
| ATOM | 2155 | CE2 | TYR | B | 107 | 15.288 | −14.252 | 2.737 | 1.00 | 11.84 | | C |
| ANISOU | 2155 | CE2 | TYR | B | 107 | 1350 | 1773 | 1374 | 110 | −441 | 37 | C |
| ATOM | 2156 | CZ | TYR | B | 107 | 15.622 | −15.548 | 3.024 | 1.00 | 12.27 | | C |
| ANISOU | 2156 | CZ | TYR | B | 107 | 1235 | 1797 | 1631 | 26 | −593 | 99 | C |
| ATOM | 2157 | OH | TYR | B | 107 | 16.466 | −15.812 | 4.080 | 1.00 | 15.75 | | O |
| ANISOU | 2157 | OH | TYR | B | 107 | 2030 | 2164 | 1791 | 79 | −980 | 215 | O |
| ATOM | 2158 | N | TYR | B | 108 | 10.702 | −13.314 | 1.330 | 1.00 | 9.82 | | N |
| ANISOU | 2158 | N | TYR | B | 108 | 1190 | 1334 | 1207 | −314 | −232 | −66 | N |
| ATOM | 2159 | CA | TYR | B | 108 | 10.414 | −12.581 | 2.544 | 1.00 | 9.46 | | C |
| ANISOU | 2159 | CA | TYR | B | 108 | 1069 | 1416 | 1108 | −26 | −119 | 120 | C |
| ATOM | 2160 | C | TYR | B | 108 | 8.996 | −12.784 | 3.065 | 1.00 | 11.03 | | C |
| ANISOU | 2160 | C | TYR | B | 108 | 1131 | 1747 | 1314 | −306 | −49 | 239 | C |
| ATOM | 2161 | O | TYR | B | 108 | 8.735 | −12.549 | 4.259 | 1.00 | 13.35 | | O |
| ANISOU | 2161 | O | TYR | B | 108 | 1226 | 2440 | 1407 | −288 | 39 | −224 | O |
| ATOM | 2162 | CB | TYR | B | 108 | 10.714 | −11.086 | 2.367 | 1.00 | 9.69 | | C |
| ANISOU | 2162 | CB | TYR | B | 108 | 921 | 1483 | 1277 | −157 | 144 | −145 | C |
| ATOM | 2163 | CG | TYR | B | 108 | 12.201 | −10.847 | 2.300 | 1.00 | 9.44 | | C |
| ANISOU | 2163 | CG | TYR | B | 108 | 858 | 1791 | 937 | −142 | 49 | −274 | C |
| ATOM | 2164 | CD1 | TYR | B | 108 | 13.005 | −10.926 | 3.425 | 1.00 | 11.22 | | C |
| ANISOU | 2164 | CD1 | TYR | B | 108 | 1130 | 2204 | 927 | −158 | −29 | −461 | C |
| ATOM | 2165 | CD2 | TYR | B | 108 | 12.838 | −10.530 | 1.114 | 1.00 | 9.73 | | C |
| ANISOU | 2165 | CD2 | TYR | B | 108 | 1125 | 1471 | 1099 | −525 | 81 | −52 | C |
| ATOM | 2166 | CE1 | TYR | B | 108 | 14.368 | −10.696 | 3.354 | 1.00 | 11.33 | | C |
| ANISOU | 2166 | CE1 | TYR | B | 108 | 1157 | 2022 | 1127 | −455 | −249 | 56 | C |
| ATOM | 2167 | CE2 | TYR | B | 108 | 14.203 | −10.303 | 1.032 | 1.00 | 11.77 | | C |
| ANISOU | 2167 | CE2 | TYR | B | 108 | 1136 | 2328 | 1008 | −512 | 113 | 19 | C |
| ATOM | 2168 | CZ | TYR | B | 108 | 14.965 | −10.387 | 2.175 | 1.00 | 11.26 | | C |
| ANISOU | 2168 | CZ | TYR | B | 108 | 1281 | 1768 | 1230 | −617 | −124 | −156 | C |
| ATOM | 2169 | OH | TYR | B | 108 | 16.320 | −10.192 | 2.082 | 1.00 | 12.69 | | O |
| ANISOU | 2169 | OH | TYR | B | 108 | 1197 | 2160 | 1463 | −471 | 32 | −177 | O |
| ATOM | 2170 | N | ALA | B | 109 | 8.056 | −13.186 | 2.225 | 1.00 | 10.18 | | N |
| ANISOU | 2170 | N | ALA | B | 109 | 1021 | 1541 | 1304 | −8 | 113 | −119 | N |
| ATOM | 2171 | CA | ALA | B | 109 | 6.674 | −13.348 | 2.648 | 1.00 | 12.05 | | C |
| ANISOU | 2171 | CA | ALA | B | 109 | 1113 | 2252 | 1212 | −485 | 56 | 144 | C |
| ATOM | 2172 | C | ALA | B | 109 | 6.564 | −14.771 | 3.163 | 1.00 | 16.39 | | C |
| ANISOU | 2172 | C | ALA | B | 109 | 2233 | 2546 | 1449 | −898 | −141 | 485 | C |
| ATOM | 2173 | O | ALA | B | 109 | 7.391 | −15.649 | 2.850 | 1.00 | 20.59 | | O |
| ANISOU | 2173 | O | ALA | B | 109 | 2229 | 2290 | 3304 | −561 | −1188 | 500 | O |
| ATOM | 2174 | CB | ALA | B | 109 | 5.710 | −13.055 | 1.521 | 1.00 | 13.07 | | C |
| ANISOU | 2174 | CB | ALA | B | 109 | 1031 | 2271 | 1663 | −188 | −106 | −81 | C |
| ATOM | 2175 | N | ASP | B | 110 | 5.572 | −15.184 | 3.900 | 1.00 | 22.46 | | N |
| ANISOU | 2175 | N | ASP | B | 110 | 3095 | 3792 | 1646 | −1878 | −118 | 887 | N |
| ATOM | 2176 | CA | ASP | B | 110 | 5.842 | −16.649 | 4.130 | 1.00 | 34.65 | | C |
| ANISOU | 2176 | CA | ASP | B | 110 | 5217 | 3963 | 3987 | −2593 | −2381 | 2522 | C |
| ATOM | 2177 | C | ASP | B | 110 | 4.581 | −17.479 | 4.460 | 1.00 | 29.59 | | C |
| ANISOU | 2177 | C | ASP | B | 110 | 3322 | 3246 | 4676 | −1239 | −3052 | 2622 | C |
| ATOM | 2178 | O | ASP | B | 110 | 3.721 | −16.844 | 5.093 | 1.00 | 48.82 | | O |
| ANISOU | 2178 | O | ASP | B | 110 | 4824 | 7179 | 6546 | −1313 | −1725 | 74 | O |
| ATOM | 2179 | CB | ASP | B | 110 | 6.808 | −16.965 | 5.236 | 1.00 | 33.73 | | C |
| ANISOU | 2179 | CB | ASP | B | 110 | 3757 | 4486 | 4573 | −2888 | −2419 | 1922 | C |
| ATOM | 2180 | CG | ASP | B | 110 | 8.304 | −16.976 | 5.085 | 1.00 | 28.48 | | C |
| ANISOU | 2180 | CG | ASP | B | 110 | 4270 | 4713 | 1840 | −1814 | −669 | 1634 | C |
| ATOM | 2181 | OD1 | ASP | B | 110 | 8.781 | −17.360 | 3.997 | 1.00 | 65.01 | | O |
| ANISOU | 2181 | OD1 | ASP | B | 110 | 11894 | 7515 | 5291 | −4706 | 4503 | −638 | O |
| ATOM | 2182 | OD2 | ASP | B | 110 | 8.921 | −16.456 | 5.988 | 1.00 | 27.80 | | O |
| ANISOU | 2182 | OD2 | ASP | B | 110 | 3258 | 3245 | 4061 | −1612 | −2699 | 2676 | O |
| ATOM | 2183 | N | GLU | B | 115 | 4.144 | −20.544 | −6.224 | 1.00 | 35.30 | | N |
| ANISOU | 2183 | N | GLU | B | 115 | 2277 | 3119 | 8014 | −868 | 785 | −833 | N |
| ATOM | 2184 | CA | GLU | B | 115 | 3.926 | −22.038 | −6.218 | 1.00 | 40.62 | | C |
| ANISOU | 2184 | CA | GLU | B | 115 | 3321 | 2807 | 9304 | 48 | −1010 | −196 | C |
| ATOM | 2185 | C | GLU | B | 115 | 2.518 | −22.341 | −6.710 | 1.00 | 32.06 | | C |
| ANISOU | 2185 | C | GLU | B | 115 | 3514 | 2122 | 6547 | −587 | −367 | −482 | C |
| ATOM | 2186 | O | GLU | B | 115 | 2.236 | −22.701 | −7.852 | 1.00 | 40.30 | | O |
| ANISOU | 2186 | O | GLU | B | 115 | 4943 | 3661 | 6708 | −562 | 117 | −1544 | O |
| ATOM | 2187 | CB | GLU | B | 115 | 4.187 | −22.661 | −4.864 | 1.00 | 41.70 | | C |
| ANISOU | 2187 | CB | GLU | B | 115 | 3122 | 3221 | 9501 | 818 | −1576 | −312 | C |
| ATOM | 2188 | CG | GLU | B | 115 | 4.429 | −21.767 | −3.662 | 1.00 | 48.19 | | C |
| ANISOU | 2188 | CG | GLU | B | 115 | 3622 | 4465 | 10222 | 2855 | −3127 | −1285 | C |
| ATOM | 2189 | CD | GLU | B | 115 | 3.268 | −21.637 | −2.739 | 1.00 | 48.79 | | C |
| ANISOU | 2189 | CD | GLU | B | 115 | 4156 | 4802 | 9578 | 3198 | −3120 | −1306 | C |
| ATOM | 2190 | OE1 | GLU | B | 115 | 2.129 | −21.507 | −3.267 | 1.00 | 34.61 | | O |
| ANISOU | 2190 | OE1 | GLU | B | 115 | 3063 | 1947 | 8142 | −977 | −2185 | 1590 | O |
| ATOM | 2191 | OE2 | GLU | B | 115 | 3.604 | −21.626 | −1.545 | 1.00 | 55.35 | | O |
| ANISOU | 2191 | OE2 | GLU | B | 115 | 3920 | 7116 | 9994 | 3264 | −3667 | −2621 | O |
| ATOM | 2192 | N | SER | B | 116 | 1.516 | −22.165 | −5.846 | 1.00 | 28.69 | | N |
| ANISOU | 2192 | N | SER | B | 116 | 3400 | 2009 | 5492 | 486 | −969 | 286 | N |
| ATOM | 2193 | CA | SER | B | 116 | 0.160 | −21.955 | −6.325 | 1.00 | 26.37 | | C |
| ANISOU | 2193 | CA | SER | B | 116 | 3450 | 1709 | 4861 | 18 | −1191 | −50 | C |
| ATOM | 2194 | C | SER | B | 116 | −0.273 | −20.541 | −5.928 | 1.00 | 20.80 | | C |
| ANISOU | 2194 | C | SER | B | 116 | 1881 | 2187 | 3837 | −205 | −411 | −465 | C |
| ATOM | 2195 | O | SER | B | 116 | −1.361 | −20.096 | −6.246 | 1.00 | 21.67 | | O |
| ANISOU | 2195 | O | SER | B | 116 | 2074 | 2233 | 3925 | −246 | −989 | −798 | O |
| ATOM | 2196 | CB | SER | B | 116 | −0.804 | −23.004 | −5.783 | 1.00 | 29.29 | | C |
| ANISOU | 2196 | CB | SER | B | 116 | 4054 | 2318 | 4755 | −469 | −1692 | 662 | C |
| ATOM | 2197 | OG | SER | B | 116 | −0.787 | −22.969 | −4.352 | 1.00 | 37.18 | | O |
| ANISOU | 2197 | OG | SER | B | 116 | 6510 | 2886 | 4730 | −2431 | −2304 | 662 | O |
| ATOM | 2198 | N | PHE | B | 117 | 0.569 | −19.790 | −5.238 | 1.00 | 20.71 | | N |
| ANISOU | 2198 | N | PHE | B | 117 | 1915 | 1830 | 4125 | 171 | −1170 | −24 | N |
| ATOM | 2199 | CA | PHE | B | 117 | 0.245 | −18.461 | −4.802 | 1.00 | 16.74 | | C |
| ANISOU | 2199 | CA | PHE | B | 117 | 1660 | 1943 | 2757 | 68 | −841 | 28 | C |
| ATOM | 2200 | C | PHE | B | 117 | −0.096 | −17.565 | −5.978 | 1.00 | 14.71 | | C |
| ANISOU | 2200 | C | PHE | B | 117 | 1459 | 1752 | 2379 | −65 | −751 | −161 | C |
| ATOM | 2201 | O | PHE | B | 117 | 0.583 | −17.586 | −6.996 | 1.00 | 16.91 | | O |
| ANISOU | 2201 | O | PHE | B | 117 | 1692 | 1951 | 2783 | −18 | −401 | −185 | O |
| ATOM | 2202 | CB | PHE | B | 117 | 1.432 | −17.883 | −4.007 | 1.00 | 16.56 | | C |
| ANISOU | 2202 | CB | PHE | B | 117 | 1513 | 1962 | 2818 | 303 | −952 | 121 | C |
| ATOM | 2203 | CG | PHE | B | 117 | 1.049 | −16.586 | −3.330 | 1.00 | 15.63 | | C |
| ANISOU | 2203 | CG | PHE | B | 117 | 979 | 2147 | 2812 | 16 | −658 | −100 | C |
| ATOM | 2204 | CD1 | PHE | B | 117 | 0.476 | −16.582 | −2.085 | 1.00 | 17.06 | | C |
| ANISOU | 2204 | CD1 | PHE | B | 117 | 1367 | 2409 | 2705 | −101 | −709 | 51 | C |
| ATOM | 2205 | CD2 | PHE | B | 117 | 1.280 | −15.371 | −3.962 | 1.00 | 15.64 | | C |
| ANISOU | 2205 | CD2 | PHE | B | 117 | 1635 | 1969 | 2336 | 125 | −608 | −321 | C |
| ATOM | 2206 | CE1 | PHE | B | 117 | 0.127 | −15.418 | −1.477 | 1.00 | 17.10 | | C |
| ANISOU | 2206 | CE1 | PHE | B | 117 | 1249 | 2650 | 2600 | 13 | −704 | −184 | C |
| ATOM | 2207 | CE2 | PHE | B | 117 | 0.892 | −14.166 | −3.382 | 1.00 | 14.65 | | C |
| ANISOU | 2207 | CE2 | PHE | B | 117 | 1005 | 2167 | 2393 | 159 | −662 | −509 | C |
| ATOM | 2208 | CZ | PHE | B | 117 | 0.306 | −14.213 | −2.139 | 1.00 | 15.80 | | C |
| ANISOU | 2208 | CZ | PHE | B | 117 | 884 | 2446 | 2673 | 85 | −391 | −411 | C |
| ATOM | 2209 | N | PRO | B | 118 | −1.155 | −16.756 | −5.876 | 1.00 | 13.11 | | N |
| ANISOU | 2209 | N | PRO | B | 118 | 1355 | 1538 | 2090 | −288 | −698 | −68 | N |
| ATOM | 2210 | CA | PRO | B | 118 | −1.550 | −15.925 | −7.007 | 1.00 | 13.73 | | C |
| ANISOU | 2210 | CA | PRO | B | 118 | 1303 | 1689 | 2225 | −242 | −716 | 65 | C |
| ATOM | 2211 | C | PRO | B | 118 | −0.751 | −14.636 | −7.157 | 1.00 | 11.94 | | C |
| ANISOU | 2211 | C | PRO | B | 118 | 1262 | 1553 | 1723 | −108 | −275 | −256 | C |
| ATOM | 2212 | O | PRO | B | 118 | −0.606 | −13.845 | −6.232 | 1.00 | 12.57 | | O |
| ANISOU | 2212 | O | PRO | B | 118 | 1470 | 1800 | 1505 | −161 | 4 | −349 | O |
| ATOM | 2213 | CB | PRO | B | 118 | −3.003 | −15.538 | −6.701 | 1.00 | 16.49 | | C |
| ANISOU | 2213 | CB | PRO | B | 118 | 1156 | 2714 | 2395 | −313 | −675 | 365 | C |
| ATOM | 2214 | CG | PRO | B | 118 | −3.182 | −15.743 | −5.263 | 1.00 | 17.85 | | C |
| ANISOU | 2214 | CG | PRO | B | 118 | 1643 | 3079 | 2062 | 365 | −776 | −423 | C |
| ATOM | 2215 | CD | PRO | B | 118 | −2.119 | −16.665 | −4.769 | 1.00 | 15.17 | | C |
| ANISOU | 2215 | CD | PRO | B | 118 | 1695 | 1933 | 2136 | −59 | −557 | −68 | C |
| ATOM | 2216 | N | PHE | B | 119 | −0.261 | −14.439 | −8.364 | 1.00 | 10.60 | | N |
| ANISOU | 2216 | N | PHE | B | 119 | 1281 | 1359 | 1387 | 152 | −506 | −344 | N |
| ATOM | 2217 | CA | PHE | B | 119 | 0.299 | −13.232 | −8.857 | 1.00 | 10.42 | | C |
| ANISOU | 2217 | CA | PHE | B | 119 | 1251 | 1465 | 1243 | 96 | −506 | −341 | C |
| ATOM | 2218 | C | PHE | B | 119 | −0.567 | −12.707 | −10.010 | 1.00 | 10.23 | | C |
| ANISOU | 2218 | C | PHE | B | 119 | 1589 | 1363 | 933 | 196 | −406 | −504 | C |
| ATOM | 2219 | O | PHE | B | 119 | −1.119 | −13.502 | −10.787 | 1.00 | 11.50 | | O |
| ANISOU | 2219 | O | PHE | B | 119 | 1437 | 1498 | 1437 | −1 | −709 | −449 | O |
| ATOM | 2220 | CB | PHE | B | 119 | 1.729 | −13.442 | −9.363 | 1.00 | 12.36 | | C |
| ANISOU | 2220 | CB | PHE | B | 119 | 1405 | 1939 | 1351 | 123 | −256 | −412 | C |
| ATOM | 2221 | CG | PHE | B | 119 | 2.706 | −13.868 | −8.281 | 1.00 | 12.43 | | C |
| ANISOU | 2221 | CG | PHE | B | 119 | 1168 | 2256 | 1301 | 434 | −135 | −539 | C |
| ATOM | 2222 | CD1 | PHE | B | 119 | 3.444 | −12.900 | −7.592 | 1.00 | 12.90 | | C |
| ANISOU | 2222 | CD1 | PHE | B | 119 | 1228 | 2327 | 1347 | 207 | −231 | −256 | C |
| ATOM | 2223 | CD2 | PHE | B | 119 | 2.902 | −15.184 | −7.935 | 1.00 | 14.66 | | C |
| ANISOU | 2223 | CD2 | PHE | B | 119 | 1700 | 2255 | 1614 | 482 | −606 | −584 | C |
| ATOM | 2224 | CE1 | PHE | B | 119 | 4.347 | −13.240 | −6.613 | 1.00 | 14.69 | | C |
| ANISOU | 2224 | CE1 | PHE | B | 119 | 1407 | 2506 | 1669 | 533 | −504 | −487 | C |
| ATOM | 2225 | CE2 | PHE | B | 119 | 3.790 | −15.516 | −6.919 | 1.00 | 15.23 | | C |
| ANISOU | 2225 | CE2 | PHE | B | 119 | 2043 | 2459 | 1285 | 719 | −552 | −712 | C |
| ATOM | 2226 | CZ | PHE | B | 119 | 4.498 | −14.547 | −6.274 | 1.00 | 13.73 | | C |
| ANISOU | 2226 | CZ | PHE | B | 119 | 1330 | 2451 | 1435 | 665 | −370 | −578 | C |
| ATOM | 2227 | N | VAL | B | 120 | −0.660 | −11.430 | −10.169 | 1.00 | 8.29 | | N |
| ANISOU | 2227 | N | VAL | B | 120 | 946 | 1367 | 837 | −135 | −126 | −243 | N |
| ATOM | 2228 | CA | VAL | B | 120 | −1.211 | −10.710 | −11.303 | 1.00 | 7.34 | | C |
| ANISOU | 2228 | CA | VAL | B | 120 | 924 | 1232 | 632 | −155 | −27 | −410 | C |
| ATOM | 2229 | C | VAL | B | 120 | −0.169 | −9.739 | −11.823 | 1.00 | 8.89 | | C |
| ANISOU | 2229 | C | VAL | B | 120 | 1086 | 1422 | 872 | −359 | −194 | −198 | C |
| ATOM | 2230 | O | VAL | B | 120 | 0.511 | −9.090 | −10.994 | 1.00 | 9.88 | | O |
| ANISOU | 2230 | O | VAL | B | 120 | 831 | 2011 | 913 | −451 | −11 | −528 | O |
| ATOM | 2231 | CB | VAL | B | 120 | −2.518 | −10.022 | −10.901 | 1.00 | 8.98 | | C |
| ANISOU | 2231 | CB | VAL | B | 120 | 1067 | 1426 | 919 | 65 | 38 | −253 | C |
| ATOM | 2232 | CG1 | VAL | B | 120 | −3.051 | −9.179 | −12.070 | 1.00 | 10.19 | | C |
| ANISOU | 2232 | CG1 | VAL | B | 120 | 1129 | 1732 | 1012 | −20 | −191 | −154 | C |
| ATOM | 2233 | CG2 | VAL | B | 120 | −3.567 | −11.029 | −10.448 | 1.00 | 10.75 | | C |
| ANISOU | 2233 | CG2 | VAL | B | 120 | 870 | 1888 | 1325 | 26 | 86 | −24 | C |
| ATOM | 2234 | N | ILE | B | 121 | 0.021 | −9.616 | −13.124 | 1.00 | 7.95 | | N |
| ANISOU | 2234 | N | ILE | B | 121 | 784 | 1379 | 858 | −243 | −215 | −73 | N |
| ATOM | 2235 | CA | ILE | B | 121 | 1.102 | −8.831 | −13.701 | 1.00 | 7.71 | | C |
| ANISOU | 2235 | CA | ILE | B | 121 | 746 | 1146 | 1037 | −190 | −173 | −217 | C |
| ATOM | 2236 | C | ILE | B | 121 | 0.477 | −7.621 | −14.388 | 1.00 | 7.93 | | C |
| ANISOU | 2236 | C | ILE | B | 121 | 937 | 1186 | 892 | −147 | −229 | −170 | C |
| ATOM | 2237 | O | ILE | B | 121 | −0.464 | −7.803 | −15.161 | 1.00 | 9.06 | | O |
| ANISOU | 2237 | O | ILE | B | 121 | 855 | 1626 | 960 | 19 | −227 | −421 | O |
| ATOM | 2238 | CB | ILE | B | 121 | 1.906 | −9.658 | −14.697 | 1.00 | 9.17 | | C |
| ANISOU | 2238 | CB | ILE | B | 121 | 816 | 1498 | 1171 | −43 | −112 | −256 | C |
| ATOM | 2239 | CG1 | ILE | B | 121 | 2.334 | −11.006 | −14.089 | 1.00 | 10.82 | | C |
| ANISOU | 2239 | CG1 | ILE | B | 121 | 1436 | 1769 | 905 | 511 | −193 | −254 | C |
| ATOM | 2240 | CG2 | ILE | B | 121 | 3.098 | −8.867 | −15.231 | 1.00 | 11.29 | | C |
| ANISOU | 2240 | CG2 | ILE | B | 121 | 1038 | 2081 | 1170 | −383 | 119 | −574 | C |
| ATOM | 2241 | CD1 | ILE | B | 121 | 3.238 | −10.929 | −12.919 | 1.00 | 13.08 | | C |
| ANISOU | 2241 | CD1 | ILE | B | 121 | 1440 | 2172 | 1356 | 255 | −489 | −357 | C |
| ATOM | 2242 | N | LEU | B | 122 | 1.002 | −6.459 | −14.059 | 1.00 | 7.80 | | N |
| ANISOU | 2242 | N | LEU | B | 122 | 695 | 1180 | 1091 | −161 | −5 | −113 | N |
| ATOM | 2243 | CA | LEU | B | 122 | 0.524 | −5.201 | −14.622 | 1.00 | 7.81 | | C |
| ANISOU | 2243 | CA | LEU | B | 122 | 798 | 1167 | 1002 | −104 | −96 | −182 | C |
| ATOM | 2244 | C | LEU | B | 122 | 1.601 | −4.539 | −15.449 | 1.00 | 8.87 | | C |
| ANISOU | 2244 | C | LEU | B | 122 | 742 | 1485 | 1144 | −173 | −258 | 183 | C |
| ATOM | 2245 | O | LEU | B | 122 | 2.690 | −4.271 | −14.926 | 1.00 | 10.46 | | O |
| ANISOU | 2245 | O | LEU | B | 122 | 807 | 1871 | 1296 | −239 | −345 | −55 | O |
| ATOM | 2246 | CB | LEU | B | 122 | 0.051 | −4.250 | −13.536 | 1.00 | 10.55 | | C |
| ANISOU | 2246 | CB | LEU | B | 122 | 914 | 1590 | 1504 | 172 | −145 | −579 | C |
| ATOM | 2247 | CG | LEU | B | 122 | −0.934 | −4.720 | −12.477 | 1.00 | 19.27 | | C |
| ANISOU | 2247 | CG | LEU | B | 122 | 2064 | 3180 | 2078 | −503 | 902 | −1301 | C |
| ATOM | 2248 | CD1 | LEU | B | 122 | −1.453 | −3.485 | −11.710 | 1.00 | 19.82 | | C |
| ANISOU | 2248 | CD1 | LEU | B | 122 | 2380 | 2683 | 2467 | −848 | 1360 | −1068 | C |
| ATOM | 2249 | CD2 | LEU | B | 122 | −2.044 | −5.515 | −13.019 | 1.00 | 24.69 | | C |
| ANISOU | 2249 | CD2 | LEU | B | 122 | 2292 | 3892 | 3196 | −1322 | 1596 | −1901 | C |
| ATOM | 2250 | N | GLY | B | 123 | 1.321 | −4.225 | −16.685 | 1.00 | 8.85 | | N |
| ANISOU | 2250 | N | GLY | B | 123 | 1002 | 1407 | 952 | −189 | −231 | −106 | N |
| ATOM | 2251 | CA | GLY | B | 123 | 2.170 | −3.425 | −17.569 | 1.00 | 8.95 | | C |
| ANISOU | 2251 | CA | GLY | B | 123 | 913 | 1547 | 941 | 35 | −157 | 35 | C |
| ATOM | 2252 | C | GLY | B | 123 | 1.543 | −2.039 | −17.670 | 1.00 | 9.23 | | C |
| ANISOU | 2252 | C | GLY | B | 123 | 721 | 1475 | 1310 | −37 | −83 | 98 | C |
| ATOM | 2253 | O | GLY | B | 123 | 0.577 | −1.884 | −18.397 | 1.00 | 10.63 | | O |
| ANISOU | 2253 | O | GLY | B | 123 | 898 | 1485 | 1657 | 12 | −279 | −28 | O |
| ATOM | 2254 | N | ASN | B | 124 | 2.059 | −1.104 | −16.883 | 1.00 | 9.77 | | N |
| ANISOU | 2254 | N | ASN | B | 124 | 996 | 1467 | 1248 | −44 | −125 | 108 | N |
| ATOM | 2255 | CA | ASN | B | 124 | 1.479 | 0.214 | −16.720 | 1.00 | 9.70 | | C |
| ANISOU | 2255 | CA | ASN | B | 124 | 797 | 1465 | 1423 | −41 | −345 | 70 | C |
| ATOM | 2256 | C | ASN | B | 124 | 2.030 | 1.233 | −17.722 | 1.00 | 9.98 | | C |
| ANISOU | 2256 | C | ASN | B | 124 | 769 | 1388 | 1635 | −229 | −455 | 83 | C |
| ATOM | 2257 | O | ASN | B | 124 | 3.046 | 1.064 | −18.385 | 1.00 | 10.60 | | O |
| ANISOU | 2257 | O | ASN | B | 124 | 1060 | 1541 | 1426 | −166 | −299 | 100 | O |
| ATOM | 2258 | CB | ASN | B | 124 | 1.625 | 0.693 | −15.280 | 1.00 | 10.51 | | C |
| ANISOU | 2258 | CB | ASN | B | 124 | 1159 | 1242 | 1592 | −265 | −390 | −34 | C |
| ATOM | 2259 | CG | ASN | B | 124 | 0.748 | 1.891 | −14.949 | 1.00 | 10.30 | | C |
| ANISOU | 2259 | CG | ASN | B | 124 | 1119 | 1466 | 1329 | −134 | −327 | 51 | C |
| ATOM | 2260 | OD1 | ASN | B | 124 | −0.414 | 1.900 | −15.337 | 1.00 | 11.80 | | O |
| ANISOU | 2260 | OD1 | ASN | B | 124 | 1125 | 1370 | 1988 | −234 | −421 | 395 | O |
| ATOM | 2261 | ND2 | ASN | B | 124 | 1.346 | 2.855 | −14.254 | 1.00 | 11.41 | | N |
| ANISOU | 2261 | ND2 | ASN | B | 124 | 1311 | 1500 | 1525 | 14 | −365 | −164 | N |
| ATOM | 2262 | N | LYS | B | 125 | 1.299 | 2.323 | −17.863 | 1.00 | 10.81 | | N |
| ANISOU | 2262 | N | LYS | B | 125 | 1122 | 1380 | 1605 | −129 | −313 | 36 | N |
| ATOM | 2263 | CA | LYS | B | 125 | 1.605 | 3.499 | −18.657 | 1.00 | 11.91 | | C |
| ANISOU | 2263 | CA | LYS | B | 125 | 1352 | 1488 | 1685 | −51 | −278 | 219 | C |
| ATOM | 2264 | C | LYS | B | 125 | 1.372 | 3.249 | −20.132 | 1.00 | 13.60 | | C |
| ANISOU | 2264 | C | LYS | B | 125 | 1252 | 2271 | 1646 | 44 | −303 | 216 | C |
| ATOM | 2265 | O | LYS | B | 125 | 2.093 | 3.749 | −21.002 | 1.00 | 14.74 | | O |
| ANISOU | 2265 | O | LYS | B | 125 | 2013 | 1923 | 1666 | −242 | −212 | 177 | O |
| ATOM | 2266 | CB | LYS | B | 125 | 3.047 | 4.046 | −18.442 | 1.00 | 10.89 | | C |
| ANISOU | 2266 | CB | LYS | B | 125 | 1297 | 1087 | 1756 | 16 | −116 | 137 | C |
| ATOM | 2267 | CG | LYS | B | 125 | 3.365 | 4.275 | −16.972 | 1.00 | 12.08 | | C |
| ANISOU | 2267 | CG | LYS | B | 125 | 1564 | 1186 | 1841 | −141 | −563 | 541 | C |
| ATOM | 2268 | CD | LYS | B | 125 | 4.559 | 5.197 | −16.788 | 1.00 | 12.35 | | C |
| ANISOU | 2268 | CD | LYS | B | 125 | 1458 | 1442 | 1792 | −200 | −362 | 413 | C |
| ATOM | 2269 | CE | LYS | B | 125 | 5.072 | 5.293 | −15.385 | 1.00 | 12.99 | | C |
| ANISOU | 2269 | CE | LYS | B | 125 | 1438 | 1711 | 1786 | −132 | −284 | 108 | C |
| ATOM | 2270 | NZ | LYS | B | 125 | 6.218 | 6.206 | −15.195 | 1.00 | 15.29 | | N |
| ANISOU | 2270 | NZ | LYS | B | 125 | 1799 | 1772 | 2238 | −346 | −699 | 388 | N |
| ATOM | 2271 | N | ILE | B | 126 | 0.326 | 2.497 | −20.463 | 1.00 | 13.42 | | N |
| ANISOU | 2271 | N | ILE | B | 126 | 1793 | 1608 | 1699 | −5 | −425 | 251 | N |
| ATOM | 2272 | CA | ILE | B | 126 | 0.149 | 2.180 | −21.884 | 1.00 | 13.79 | | C |
| ANISOU | 2272 | CA | ILE | B | 126 | 1811 | 1711 | 1717 | 203 | −472 | 165 | C |
| ATOM | 2273 | C | ILE | B | 126 | −0.365 | 3.352 | −22.722 | 1.00 | 15.19 | | C |
| ANISOU | 2273 | C | ILE | B | 126 | 2388 | 1849 | 1535 | 336 | −67 | 392 | C |
| ATOM | 2274 | O | ILE | B | 126 | −0.352 | 3.231 | −23.952 | 1.00 | 17.47 | | O |
| ANISOU | 2274 | O | ILE | B | 126 | 2642 | 2483 | 1511 | −146 | −462 | 241 | O |
| ATOM | 2275 | CB | ILE | B | 126 | −0.811 | 1.006 | −22.089 | 1.00 | 14.64 | | C |
| ANISOU | 2275 | CB | ILE | B | 126 | 2183 | 2018 | 1360 | −207 | −239 | 168 | C |
| ATOM | 2276 | CG1 | ILE | B | 126 | −2.215 | 1.228 | −21.546 | 1.00 | 18.46 | | C |
| ANISOU | 2276 | CG1 | ILE | B | 126 | 2032 | 3153 | 1827 | −297 | −273 | 126 | C |
| ATOM | 2277 | CG2 | ILE | B | 126 | −0.100 | −0.191 | −21.489 | 1.00 | 17.92 | | C |
| ANISOU | 2277 | CG2 | ILE | B | 126 | 2792 | 1796 | 2221 | −173 | −349 | 199 | C |
| ATOM | 2278 | CD1 | ILE | B | 126 | −3.206 | 0.155 | −21.963 | 1.00 | 34.91 | | C |
| ANISOU | 2278 | CD1 | ILE | B | 126 | 2879 | 5883 | 4502 | −2058 | −1959 | 823 | C |
| ATOM | 2279 | N | ASP | B | 127 | −0.741 | 4.408 | −22.016 | 1.00 | 15.57 | | N |
| ANISOU | 2279 | N | ASP | B | 127 | 2232 | 1944 | 1741 | 534 | −349 | 306 | N |
| ATOM | 2280 | CA | ASP | B | 127 | −1.051 | 5.660 | −22.711 | 1.00 | 19.16 | | C |
| ANISOU | 2280 | CA | ASP | B | 127 | 3057 | 1882 | 2340 | 579 | −626 | 252 | C |
| ATOM | 2281 | C | ASP | B | 127 | 0.147 | 6.329 | −23.374 | 1.00 | 23.03 | | C |
| ANISOU | 2281 | C | ASP | B | 127 | 3940 | 2450 | 2358 | 176 | −504 | 1026 | C |
| ATOM | 2282 | O | ASP | B | 127 | −0.106 | 7.216 | −24.231 | 1.00 | 32.50 | | O |
| ANISOU | 2282 | O | ASP | B | 127 | 6001 | 2564 | 3784 | 645 | −509 | 1773 | O |
| ATOM | 2283 | CB | ASP | B | 127 | −1.721 | 6.638 | −21.718 | 1.00 | 20.63 | | C |
| ANISOU | 2283 | CB | ASP | B | 127 | 3167 | 1961 | 2712 | 672 | −1103 | −260 | C |
| ATOM | 2284 | CG | ASP | B | 127 | −0.779 | 6.920 | −20.547 | 1.00 | 17.22 | | C |
| ANISOU | 2284 | CG | ASP | B | 127 | 2648 | 1696 | 2199 | 124 | −623 | 435 | C |
| ATOM | 2285 | OD1 | ASP | B | 127 | −0.707 | 6.116 | −19.605 | 1.00 | 17.70 | | O |
| ANISOU | 2285 | OD1 | ASP | B | 127 | 2559 | 1969 | 2196 | 402 | −131 | 556 | O |
| ATOM | 2286 | OD2 | ASP | B | 127 | −0.124 | 7.976 | −20.599 | 1.00 | 18.75 | | O |
| ANISOU | 2286 | OD2 | ASP | B | 127 | 2645 | 1774 | 2706 | 53 | −459 | 410 | O |
| ATOM | 2287 | N | ILE | B | 128 | 1.376 | 5.970 | −23.057 | 1.00 | 20.55 | | N |
| ANISOU | 2287 | N | ILE | B | 128 | 3418 | 2058 | 2330 | −546 | −304 | 673 | N |
| ATOM | 2288 | CA | ILE | B | 128 | 2.590 | 6.554 | −23.645 | 1.00 | 22.91 | | C |
| ANISOU | 2288 | CA | ILE | B | 128 | 4238 | 1720 | 2745 | −740 | 227 | 877 | C |
| ATOM | 2289 | C | ILE | B | 128 | 2.875 | 5.989 | −25.015 | 1.00 | 26.24 | | C |
| ANISOU | 2289 | C | ILE | B | 128 | 3657 | 3105 | 3208 | −1080 | 369 | 54 | C |
| ATOM | 2290 | O | ILE | B | 128 | 2.623 | 4.827 | −25.330 | 1.00 | 36.67 | | O |
| ANISOU | 2290 | O | ILE | B | 128 | 5994 | 4155 | 3782 | −2932 | 1699 | −874 | O |
| ATOM | 2291 | CB | ILE | B | 128 | 3.788 | 6.286 | −22.714 | 1.00 | 27.02 | | C |
| ANISOU | 2291 | CB | ILE | B | 128 | 3180 | 4037 | 3049 | −826 | 588 | 236 | C |
| ATOM | 2292 | CG1 | ILE | B | 128 | 3.616 | 6.973 | −21.359 | 1.00 | 23.36 | | C |
| ANISOU | 2292 | CG1 | ILE | B | 128 | 2640 | 3093 | 3141 | −707 | 239 | 337 | C |
| ATOM | 2293 | CG2 | ILE | B | 128 | 5.115 | 6.677 | −23.327 | 1.00 | 43.08 | | C |
| ANISOU | 2293 | CG2 | ILE | B | 128 | 3849 | 6673 | 5848 | −2171 | 2153 | −2697 | C |
| ATOM | 2294 | CD1 | ILE | B | 128 | 4.685 | 6.655 | −20.342 | 1.00 | 29.39 | | C |
| ANISOU | 2294 | CD1 | ILE | B | 128 | 3263 | 4532 | 3372 | −1032 | −162 | 1092 | C |
| ATOM | 2295 | N | SER | B | 129 | 3.438 | 6.772 | −25.908 | 1.00 | 23.12 | | N |
| ANISOU | 2295 | N | SER | B | 129 | 3573 | 2608 | 2603 | −210 | 139 | 136 | N |
| ATOM | 2296 | CA | SER | B | 129 | 3.648 | 6.328 | −27.271 | 1.00 | 28.19 | | C |
| ANISOU | 2296 | CA | SER | B | 129 | 5456 | 2515 | 2742 | 375 | 219 | 32 | C |
| ATOM | 2297 | C | SER | B | 129 | 4.935 | 5.541 | −27.421 | 1.00 | 27.98 | | C |
| ANISOU | 2297 | C | SER | B | 129 | 5804 | 2219 | 2608 | 543 | 341 | −96 | C |
| ATOM | 2298 | O | SER | B | 129 | 4.917 | 4.639 | −28.239 | 1.00 | 36.49 | | O |
| ANISOU | 2298 | O | SER | B | 129 | 6562 | 3606 | 3695 | 263 | 802 | −1384 | O |
| ATOM | 2299 | CB | SER | B | 129 | 3.647 | 7.540 | −28.218 | 1.00 | 30.11 | | C |
| ANISOU | 2299 | CB | SER | B | 129 | 5736 | 3124 | 2579 | 861 | 139 | 340 | C |
| ATOM | 2300 | OG | SER | B | 129 | 2.428 | 8.237 | −28.074 | 1.00 | 35.71 | | O |
| ANISOU | 2300 | OG | SER | B | 129 | 6222 | 4042 | 3305 | 1606 | 23 | 427 | O |
| ATOM | 2301 | N | GLU | B | 130 | 5.983 | 5.868 | −26.686 | 1.00 | 27.08 | | N |
| ANISOU | 2301 | N | GLU | B | 130 | 5766 | 2042 | 2479 | 889 | 199 | 301 | N |
| ATOM | 2302 | CA | GLU | B | 130 | 7.263 | 5.176 | −26.806 | 1.00 | 32.60 | | C |
| ANISOU | 2302 | CA | GLU | B | 130 | 5971 | 2416 | 4001 | 1154 | 186 | 472 | C |
| ATOM | 2303 | C | GLU | B | 130 | 7.369 | 3.993 | −25.851 | 1.00 | 30.31 | | C |
| ANISOU | 2303 | C | GLU | B | 130 | 6021 | 2625 | 2871 | 1400 | −41 | 159 | C |
| ATOM | 2304 | O | GLU | B | 130 | 7.701 | 4.188 | −24.674 | 1.00 | 39.51 | | O |
| ANISOU | 2304 | O | GLU | B | 130 | 8336 | 4039 | 2636 | −1512 | 489 | 136 | O |
| ATOM | 2305 | CB | GLU | B | 130 | 8.441 | 6.127 | −26.549 | 1.00 | 38.17 | | C |
| ANISOU | 2305 | CB | GLU | B | 130 | 5771 | 3585 | 5145 | 666 | 556 | 393 | C |
| ATOM | 2306 | CG | GLU | B | 130 | 8.757 | 7.166 | −27.586 | 1.00 | 40.30 | | C |
| ANISOU | 2306 | CG | GLU | B | 130 | 6423 | 3822 | 5069 | 279 | 1579 | 83 | C |
| ATOM | 2307 | CD | GLU | B | 130 | 9.237 | 6.702 | −28.946 | 1.00 | 48.84 | | C |
| ANISOU | 2307 | CD | GLU | B | 130 | 7821 | 5176 | 5561 | 139 | 2192 | −513 | C |
| ATOM | 2308 | OE1 | GLU | B | 130 | 8.423 | 6.286 | −29.798 | 1.00 | 65.04 | | O |
| ANISOU | 2308 | OE1 | GLU | B | 130 | 10149 | 7646 | 6916 | −1813 | 2307 | −3103 | O |
| ATOM | 2309 | OE2 | GLU | B | 130 | 10.467 | 6.770 | −29.175 | 1.00 | 57.83 | | O |
| ANISOU | 2309 | OE2 | GLU | B | 130 | 7500 | 7560 | 6912 | 3208 | 2263 | −477 | O |
| ATOM | 2310 | N | ARG | B | 131 | 7.154 | 2.778 | −26.365 | 1.00 | 20.15 | | N |
| ANISOU | 2310 | N | ARG | B | 131 | 2884 | 2825 | 1946 | −327 | 509 | 955 | N |
| ATOM | 2311 | CA | ARG | B | 131 | 7.285 | 1.582 | −25.536 | 1.00 | 17.93 | | C |
| ANISOU | 2311 | CA | ARG | B | 131 | 1892 | 2939 | 1982 | −399 | 481 | 1077 | C |
| ATOM | 2312 | C | ARG | B | 131 | 8.607 | 0.857 | −25.665 | 1.00 | 21.12 | | C |
| ANISOU | 2312 | C | ARG | B | 131 | 2100 | 3713 | 2210 | O | 449 | 870 | C |
| ATOM | 2313 | O | ARG | B | 131 | 9.149 | 0.843 | −26.762 | 1.00 | 25.43 | | O |
| ANISOU | 2313 | O | ARG | B | 131 | 3585 | 4073 | 2004 | 1417 | 626 | 706 | O |
| ATOM | 2314 | CB | ARG | B | 131 | 6.215 | 0.563 | −25.942 | 1.00 | 23.69 | | C |
| ANISOU | 2314 | CB | ARG | B | 131 | 2351 | 3025 | 3626 | −557 | −189 | 1142 | C |
| ATOM | 2315 | CG | ARG | B | 131 | 4.793 | 0.968 | −25.746 | 1.00 | 22.89 | | C |
| ANISOU | 2315 | CG | ARG | B | 131 | 2177 | 3257 | 3265 | −689 | −217 | 1528 | C |
| ATOM | 2316 | CD | ARG | B | 131 | 3.793 | −0.129 | −26.028 | 1.00 | 21.26 | | C |
| ANISOU | 2316 | CD | ARG | B | 131 | 2401 | 3380 | 2295 | −751 | −261 | 1327 | C |
| ATOM | 2317 | NE | ARG | B | 131 | 3.896 | −1.364 | −25.258 | 1.00 | 17.94 | | N |
| ANISOU | 2317 | NE | ARG | B | 131 | 1953 | 2775 | 2090 | −422 | 56 | 671 | N |
| ATOM | 2318 | CZ | ARG | B | 131 | 2.897 | −2.034 | −24.689 | 1.00 | 16.33 | | C |
| ANISOU | 2318 | CZ | ARG | B | 131 | 1737 | 2496 | 1973 | −226 | −20 | 735 | C |
| ATOM | 2319 | NH1 | ARG | B | 131 | 1.611 | −1.676 | −24.720 | 1.00 | 18.66 | | N |
| ANISOU | 2319 | NH1 | ARG | B | 131 | 1820 | 3196 | 2074 | 28 | 21 | 480 | N |
| ATOM | 2320 | NH2 | ARG | B | 131 | 3.166 | −3.148 | −24.035 | 1.00 | 17.03 | | N |
| ANISOU | 2320 | NH2 | ARG | B | 131 | 2639 | 1998 | 1834 | 23 | 170 | 254 | N |
| ATOM | 2321 | N | GLN | B | 132 | 9.104 | 0.256 | −24.589 | 1.00 | 16.16 | | N |
| ANISOU | 2321 | N | GLN | B | 132 | 1538 | 2486 | 2115 | −553 | 451 | 623 | N |
| ATOM | 2322 | CA | GLN | B | 132 | 10.303 | −0.591 | −24.666 | 1.00 | 17.76 | | C |
| ANISOU | 2322 | CA | GLN | B | 132 | 1611 | 2596 | 2541 | −521 | 129 | 240 | C |
| ATOM | 2323 | C | GLN | B | 132 | 9.986 | −2.060 | −24.498 | 1.00 | 17.16 | | C |
| ANISOU | 2323 | C | GLN | B | 132 | 1591 | 2582 | 2346 | −327 | 221 | 256 | C |
| ATOM | 2324 | O | GLN | B | 132 | 10.895 | −2.913 | −24.640 | 1.00 | 22.16 | | O |
| ANISOU | 2324 | O | GLN | B | 132 | 2378 | 2976 | 3066 | 317 | 741 | 975 | O |
| ATOM | 2325 | CB | GLN | B | 132 | 11.270 | −0.154 | −23.564 | 1.00 | 23.14 | | C |
| ANISOU | 2325 | CB | GLN | B | 132 | 2150 | 3263 | 3379 | −1603 | −771 | 1438 | C |
| ATOM | 2326 | CG | GLN | B | 132 | 11.728 | 1.282 | −23.688 | 1.00 | 25.56 | | C |
| ANISOU | 2326 | CG | GLN | B | 132 | 3218 | 3307 | 3188 | −1896 | −1101 | 1446 | C |
| ATOM | 2327 | CD | GLN | B | 132 | 12.772 | 1.678 | −22.653 | 1.00 | 20.08 | | C |
| ANISOU | 2327 | CD | GLN | B | 132 | 1737 | 2752 | 3142 | −884 | −447 | 841 | C |
| ATOM | 2328 | OE1 | GLN | B | 132 | 13.138 | 0.945 | −21.746 | 1.00 | 17.18 | | O |
| ANISOU | 2328 | OE1 | GLN | B | 132 | 1419 | 2522 | 2586 | −393 | −14 | 414 | O |
| ATOM | 2329 | NE2 | GLN | B | 132 | 13.279 | 2.894 | −22.812 | 1.00 | 28.30 | | N |
| ANISOU | 2329 | NE2 | GLN | B | 132 | 4099 | 3093 | 3562 | −1895 | −1325 | 1056 | N |
| ATOM | 2330 | N | VAL | B | 133 | 8.743 | −2.368 | −24.155 | 1.00 | 15.01 | | N |
| ANISOU | 2330 | N | VAL | B | 133 | 1459 | 2658 | 1585 | −566 | −457 | 676 | N |
| ATOM | 2331 | CA | VAL | B | 133 | 8.267 | −3.723 | −23.946 | 1.00 | 13.71 | | C |
| ANISOU | 2331 | CA | VAL | B | 133 | 1627 | 2403 | 1177 | −309 | −263 | 306 | C |
| ATOM | 2332 | C | VAL | B | 133 | 6.985 | −3.883 | −24.773 | 1.00 | 13.56 | | C |
| ANISOU | 2332 | C | VAL | B | 133 | 1767 | 1902 | 1483 | −389 | −478 | 397 | C |
| ATOM | 2333 | O | VAL | B | 133 | 6.055 | −3.133 | −24.507 | 1.00 | 14.89 | | O |
| ANISOU | 2333 | O | VAL | B | 133 | 1699 | 2688 | 1272 | −180 | −298 | 240 | O |
| ATOM | 2334 | CB | VAL | B | 133 | 7.978 | −4.035 | −22.469 | 1.00 | 11.45 | | C |
| ANISOU | 2334 | CB | VAL | B | 133 | 1053 | 2071 | 1227 | −118 | −49 | 245 | C |
| ATOM | 2335 | CG1 | VAL | B | 133 | 7.538 | −5.465 | −22.297 | 1.00 | 13.80 | | C |
| ANISOU | 2335 | CG1 | VAL | B | 133 | 1314 | 2074 | 1857 | −63 | 83 | 329 | C |
| ATOM | 2336 | CG2 | VAL | B | 133 | 9.221 | −3.683 | −21.665 | 1.00 | 15.29 | | C |
| ANISOU | 2336 | CG2 | VAL | B | 133 | 1777 | 2550 | 1483 | −375 | −681 | 624 | C |
| ATOM | 2337 | N | SER | B | 134 | 6.992 | −4.817 | −25.722 | 1.00 | 13.29 | | N |
| ANISOU | 2337 | N | SER | B | 134 | 1442 | 2296 | 1311 | −467 | −188 | 350 | N |
| ATOM | 2338 | CA | SER | B | 134 | 5.800 | −5.043 | −26.516 | 1.00 | 13.99 | | C |
| ANISOU | 2338 | CA | SER | B | 134 | 1561 | 2388 | 1367 | −574 | −232 | 155 | C |
| ATOM | 2339 | C | SER | B | 134 | 4.792 | −5.859 | −25.731 | 1.00 | 12.86 | | C |
| ANISOU | 2339 | C | SER | B | 134 | 1419 | 2291 | 1175 | −395 | −312 | 162 | C |
| ATOM | 2340 | O | SER | B | 134 | 5.084 | −6.615 | −24.805 | 1.00 | 12.47 | | O |
| ANISOU | 2340 | O | SER | B | 134 | 1419 | 2063 | 1256 | −296 | −196 | 76 | O |
| ATOM | 2341 | CB | SER | B | 134 | 6.152 | −5.782 | −27.805 | 1.00 | 16.24 | | C |
| ANISOU | 2341 | CB | SER | B | 134 | 2135 | 2459 | 1577 | −1234 | 186 | −98 | C |
| ATOM | 2342 | OG | SER | B | 134 | 6.533 | −7.130 | −27.487 | 1.00 | 18.32 | | O |
| ANISOU | 2342 | OG | SER | B | 134 | 2624 | 2490 | 1847 | −925 | 325 | −241 | O |
| ATOM | 2343 | N | THR | B | 135 | 3.530 | −5.768 | −26.157 | 1.00 | 13.78 | | N |
| ANISOU | 2343 | N | THR | B | 135 | 1518 | 2270 | 1449 | −621 | −490 | 209 | N |
| ATOM | 2344 | CA | THR | B | 135 | 2.480 | −6.546 | −25.513 | 1.00 | 12.89 | | C |
| ANISOU | 2344 | CA | THR | B | 135 | 1487 | 2013 | 1400 | −307 | −175 | 187 | C |
| ATOM | 2345 | C | THR | B | 135 | 2.821 | −8.019 | −25.653 | 1.00 | 11.93 | | C |
| ANISOU | 2345 | C | THR | B | 135 | 1296 | 2082 | 1156 | −340 | −58 | 42 | C |
| ATOM | 2346 | O | THR | B | 135 | 2.692 | −8.785 | −24.698 | 1.00 | 11.64 | | O |
| ANISOU | 2346 | O | THR | B | 135 | 1428 | 1939 | 1056 | −86 | −153 | −15 | O |
| ATOM | 2347 | CB | THR | B | 135 | 1.138 | −6.230 | −26.166 | 1.00 | 14.75 | | C |
| ANISOU | 2347 | CB | THR | B | 135 | 1344 | 2645 | 1615 | −6 | −41 | 176 | C |
| ATOM | 2348 | OG1 | THR | B | 135 | 0.801 | −4.842 | −26.021 | 1.00 | 16.57 | | O |
| ANISOU | 2348 | OG1 | THR | B | 135 | 1597 | 2614 | 2083 | 10 | −309 | 252 | O |
| ATOM | 2349 | CG2 | THR | B | 135 | −0.008 | −6.953 | −25.486 | 1.00 | 16.43 | | C |
| ANISOU | 2349 | CG2 | THR | B | 135 | 1556 | 2882 | 1802 | −384 | −143 | 126 | C |
| ATOM | 2350 | N | GLU | B | 136 | 3.292 | −8.437 | −26.835 | 1.00 | 12.35 | | N |
| ANISOU | 2350 | N | GLU | B | 136 | 1075 | 2452 | 1165 | −563 | 24 | −66 | N |
| ATOM | 2351 | CA | GLU | B | 136 | 3.607 | −9.819 | −27.073 | 1.00 | 14.62 | | C |
| ANISOU | 2351 | CA | GLU | B | 136 | 1577 | 2494 | 1483 | −479 | 169 | −302 | C |
| ATOM | 2352 | C | GLU | B | 136 | 4.684 | −10.347 | −26.143 | 1.00 | 13.04 | | C |
| ANISOU | 2352 | C | GLU | B | 136 | 1311 | 2235 | 1410 | −194 | 247 | −763 | C |
| ATOM | 2353 | O | GLU | B | 136 | 4.606 | −11.438 | −25.578 | 1.00 | 13.21 | | O |
| ANISOU | 2353 | O | GLU | B | 136 | 1177 | 2043 | 1799 | −161 | 27 | −784 | O |
| ATOM | 2354 | CB | GLU | B | 136 | 4.044 | −10.035 | −28.544 | 1.00 | 21.99 | | C |
| ANISOU | 2354 | CB | GLU | B | 136 | 3836 | 3280 | 1240 | −994 | −89 | −1022 | C |
| ATOM | 2355 | CG | GLU | B | 136 | 2.940 | −9.826 | −29.550 | 1.00 | 27.07 | | C |
| ANISOU | 2355 | CG | GLU | B | 136 | 3448 | 5229 | 1609 | −722 | −173 | −1693 | C |
| ATOM | 2356 | CD | GLU | B | 136 | 2.548 | −8.383 | −29.794 | 1.00 | 40.07 | | C |
| ANISOU | 2356 | CD | GLU | B | 136 | 5866 | 5640 | 3718 | −632 | −2873 | −499 | C |
| ATOM | 2357 | OE1 | GLU | B | 136 | 3.330 | −7.447 | −29.445 | 1.00 | 24.62 | | O |
| ANISOU | 2357 | OE1 | GLU | B | 136 | 2848 | 4935 | 1570 | −104 | −312 | 286 | O |
| ATOM | 2358 | OE2 | GLU | B | 136 | 1.421 | −8.220 | −30.348 | 1.00 | 61.44 | | O |
| ANISOU | 2358 | OE2 | GLU | B | 136 | 9234 | 5870 | 8240 | −2208 | −7514 | 1629 | O |
| ATOM | 2359 | N | GLU | B | 137 | 5.732 | −9.528 | −25.943 | 1.00 | 13.27 | | N |
| ANISOU | 2359 | N | GLU | B | 137 | 1398 | 2299 | 1344 | −271 | 347 | −372 | N |
| ATOM | 2360 | CA | GLU | B | 137 | 6.790 | −9.926 | −25.029 | 1.00 | 13.47 | | C |
| ANISOU | 2360 | CA | GLU | B | 137 | 1211 | 2318 | 1587 | −466 | 222 | −468 | C |
| ATOM | 2361 | C | GLU | B | 137 | 6.308 | −10.097 | −23.614 | 1.00 | 11.14 | | C |
| ANISOU | 2361 | C | GLU | B | 137 | 675 | 1984 | 1574 | −167 | 8 | −293 | C |
| ATOM | 2362 | O | GLU | B | 137 | 6.638 | −11.036 | −22.871 | 1.00 | 11.50 | | O |
| ANISOU | 2362 | O | GLU | B | 137 | 828 | 1788 | 1753 | −121 | 96 | −338 | O |
| ATOM | 2363 | CB | GLU | B | 137 | 7.888 | −8.839 | −25.094 | 1.00 | 16.19 | | C |
| ANISOU | 2363 | CB | GLU | B | 137 | 1630 | 2652 | 1870 | −805 | 455 | −389 | C |
| ATOM | 2364 | CG | GLU | B | 137 | 8.771 | −9.064 | −26.306 | 1.00 | 19.29 | | C |
| ANISOU | 2364 | CG | GLU | B | 137 | 1860 | 3307 | 2164 | −616 | 770 | −90 | C |
| ATOM | 2365 | CD | GLU | B | 137 | 9.755 | −7.903 | −26.412 | 1.00 | 23.02 | | C |
| ANISOU | 2365 | CD | GLU | B | 137 | 2318 | 3517 | 2910 | −926 | 1236 | −231 | C |
| ATOM | 2366 | OE1 | GLU | B | 137 | 9.561 | −6.824 | −25.795 | 1.00 | 28.79 | | O |
| ANISOU | 2366 | OE1 | GLU | B | 137 | 1672 | 4254 | 5014 | −1167 | 967 | −1604 | O |
| ATOM | 2367 | OE2 | GLU | B | 137 | 10.700 | −8.179 | −27.172 | 1.00 | 18.34 | | O |
| ANISOU | 2367 | OE2 | GLU | B | 137 | 1292 | 3121 | 2556 | −75 | 374 | 642 | O |
| ATOM | 2368 | N | ALA | B | 138 | 5.438 | −9.178 | −23.142 | 1.00 | 10.08 | | N |
| ANISOU | 2368 | N | ALA | B | 138 | 895 | 1643 | 1292 | −295 | −95 | −294 | N |
| ATOM | 2369 | CA | ALA | B | 138 | 4.907 | −9.267 | −21.799 | 1.00 | 8.85 | | C |
| ANISOU | 2369 | CA | ALA | B | 138 | 772 | 1400 | 1189 | −139 | −226 | −237 | C |
| ATOM | 2370 | C | ALA | B | 138 | 4.021 | −10.488 | −21.643 | 1.00 | 8.85 | | C |
| ANISOU | 2370 | C | ALA | B | 138 | 826 | 1395 | 1142 | −158 | −121 | −300 | C |
| ATOM | 2371 | O | ALA | B | 138 | 4.106 | −11.207 | −20.636 | 1.00 | 9.20 | | O |
| ANISOU | 2371 | O | ALA | B | 138 | 784 | 1374 | 1337 | −23 | −139 | −190 | O |
| ATOM | 2372 | CB | ALA | B | 138 | 4.125 | −8.033 | −21.415 | 1.00 | 10.47 | | C |
| ANISOU | 2372 | CB | ALA | B | 138 | 856 | 1435 | 1688 | −134 | −86 | −359 | C |
| ATOM | 2373 | N | GLN | B | 139 | 3.154 | −10.718 | −22.641 | 1.00 | 8.66 | | N |
| ANISOU | 2373 | N | GLN | B | 139 | 826 | 1461 | 1004 | −276 | −19 | −192 | N |
| ATOM | 2374 | CA | GLN | B | 139 | 2.299 | −11.869 | −22.586 | 1.00 | 9.06 | | C |
| ANISOU | 2374 | CA | GLN | B | 139 | 764 | 1449 | 1227 | −195 | −70 | −282 | C |
| ATOM | 2375 | C | GLN | B | 139 | 3.103 | −13.179 | −22.545 | 1.00 | 10.07 | | C |
| ANISOU | 2375 | C | GLN | B | 139 | 1032 | 1429 | 1365 | −113 | −108 | −531 | C |
| ATOM | 2376 | O | GLN | B | 139 | 2.776 | −14.112 | −21.823 | 1.00 | 10.60 | | O |
| ANISOU | 2376 | O | GLN | B | 139 | 789 | 1640 | 1601 | 67 | −191 | −175 | O |
| ATOM | 2377 | CB | GLN | B | 139 | 1.355 | −11.866 | −23.791 | 1.00 | 10.73 | | C |
| ANISOU | 2377 | CB | GLN | B | 139 | 812 | 1911 | 1356 | −171 | −224 | −570 | C |
| ATOM | 2378 | CG | GLN | B | 139 | 0.323 | −10.781 | −23.783 | 1.00 | 11.82 | | C |
| ANISOU | 2378 | CG | GLN | B | 139 | 919 | 1767 | 1805 | −219 | −494 | −458 | C |
| ATOM | 2379 | CD | GLN | B | 139 | −0.342 | −10.619 | −25.141 | 1.00 | 12.22 | | C |
| ANISOU | 2379 | CD | GLN | B | 139 | 790 | 2085 | 1768 | −220 | −470 | −568 | C |
| ATOM | 2380 | OE1 | GLN | B | 139 | 0.352 | −10.714 | −26.169 | 1.00 | 17.39 | | O |
| ANISOU | 2380 | OE1 | GLN | B | 139 | 1014 | 3729 | 1866 | −436 | −250 | −162 | O |
| ATOM | 2381 | NE2 | GLN | B | 139 | −1.612 | −10.315 | −25.129 | 1.00 | 15.72 | | N |
| ANISOU | 2381 | NE2 | GLN | B | 139 | 931 | 2534 | 2509 | 239 | −719 | −985 | N |
| ATOM | 2382 | N | ALA | B | 140 | 4.171 | −13.237 | −23.333 | 1.00 | 10.79 | | N |
| ANISOU | 2382 | N | ALA | B | 140 | 910 | 1317 | 1871 | −266 | 38 | −481 | N |
| ATOM | 2383 | CA | ALA | B | 140 | 5.013 | −14.429 | −23.344 | 1.00 | 11.89 | | C |
| ANISOU | 2383 | CA | ALA | B | 140 | 1156 | 1210 | 2151 | −281 | 295 | −539 | C |
| ATOM | 2384 | C | ALA | B | 140 | 5.650 | −14.698 | −21.993 | 1.00 | 12.23 | | C |
| ANISOU | 2384 | C | ALA | B | 140 | 705 | 1449 | 2492 | 18 | 84 | −474 | C |
| ATOM | 2385 | O | ALA | B | 140 | 5.676 | −15.846 | −21.507 | 1.00 | 12.63 | | O |
| ANISOU | 2385 | O | ALA | B | 140 | 739 | 1519 | 2541 | −85 | 222 | −321 | O |
| ATOM | 2386 | CB | ALA | B | 140 | 6.078 | −14.390 | −24.411 | 1.00 | 15.24 | | C |
| ANISOU | 2386 | CB | ALA | B | 140 | 1160 | 1918 | 2714 | −101 | 571 | −331 | C |
| ATOM | 2387 | N | TRP | B | 141 | 6.154 | −13.647 | −21.353 | 1.00 | 11.76 | | N |
| ANISOU | 2387 | N | TRP | B | 141 | 722 | 1430 | 2315 | 41 | −106 | −237 | N |
| ATOM | 2388 | CA | TRP | B | 141 | 6.774 | −13.824 | −20.063 | 1.00 | 12.40 | | C |
| ANISOU | 2388 | CA | TRP | B | 141 | 913 | 1251 | 2547 | 213 | −341 | −189 | C |
| ATOM | 2389 | C | TRP | B | 141 | 5.732 | −14.359 | −19.099 | 1.00 | 12.54 | | C |
| ANISOU | 2389 | C | TRP | B | 141 | 808 | 1910 | 2046 | 67 | −672 | −167 | C |
| ATOM | 2390 | O | TRP | B | 141 | 5.950 | −15.308 | −18.326 | 1.00 | 12.10 | | O |
| ANISOU | 2390 | O | TRP | B | 141 | 930 | 1544 | 2122 | 85 | −388 | −342 | O |
| ATOM | 2391 | CB | TRP | B | 141 | 7.406 | −12.496 | −19.580 | 1.00 | 13.38 | | C |
| ANISOU | 2391 | CB | TRP | B | 141 | 1320 | 1190 | 2574 | 228 | −496 | −278 | C |
| ATOM | 2392 | CG | TRP | B | 141 | 8.148 | −12.748 | −18.286 | 1.00 | 13.24 | | C |
| ANISOU | 2392 | CG | TRP | B | 141 | 866 | 1520 | 2644 | −100 | −418 | −66 | C |
| ATOM | 2393 | CD1 | TRP | B | 141 | 9.448 | −13.099 | −18.165 | 1.00 | 15.99 | | C |
| ANISOU | 2393 | CD1 | TRP | B | 141 | 914 | 2281 | 2883 | −170 | −537 | −246 | C |
| ATOM | 2394 | CD2 | TRP | B | 141 | 7.605 | −12.697 | −16.966 | 1.00 | 13.61 | | C |
| ANISOU | 2394 | CD2 | TRP | B | 141 | 975 | 1619 | 2576 | −289 | −531 | −279 | C |
| ATOM | 2395 | NE1 | TRP | B | 141 | 9.749 | −13.253 | −16.833 | 1.00 | 16.05 | | N |
| ANISOU | 2395 | NE1 | TRP | B | 141 | 1063 | 2046 | 2990 | −184 | −724 | 145 | N |
| ATOM | 2396 | CE2 | TRP | B | 141 | 8.640 | −13.020 | −16.077 | 1.00 | 15.47 | | C |
| ANISOU | 2396 | CE2 | TRP | B | 141 | 1352 | 1841 | 2687 | −190 | −764 | −384 | C |
| ATOM | 2397 | CE3 | TRP | B | 141 | 6.322 | −12.410 | −16.447 | 1.00 | 14.02 | | C |
| ANISOU | 2397 | CE3 | TRP | B | 141 | 1139 | 1818 | 2369 | −355 | −365 | −391 | C |
| ATOM | 2398 | CZ2 | TRP | B | 141 | 8.458 | −13.060 | −14.708 | 1.00 | 18.76 | | C |
| ANISOU | 2398 | CZ2 | TRP | B | 141 | 1750 | 2681 | 2699 | 273 | −824 | −505 | C |
| ATOM | 2399 | CZ3 | TRP | B | 141 | 6.155 | −12.456 | −15.074 | 1.00 | 16.42 | | C |
| ANISOU | 2399 | CZ3 | TRP | B | 141 | 1780 | 2131 | 2328 | 497 | −529 | −690 | C |
| ATOM | 2400 | CH2 | TRP | B | 141 | 7.214 | −12.778 | −14.221 | 1.00 | 17.14 | | C |
| ANISOU | 2400 | CH2 | TRP | B | 141 | 1878 | 2057 | 2577 | 394 | −733 | −592 | C |
| ATOM | 2401 | N | CYS | B | 142 | 4.522 | −13.798 | −19.120 | 1.00 | 11.01 | | N |
| ANISOU | 2401 | N | CYS | B | 142 | 797 | 1523 | 1863 | −50 | −497 | −352 | N |
| ATOM | 2402 | CA | CYS | B | 142 | 3.523 | −14.259 | −18.168 | 1.00 | 11.09 | | C |
| ANISOU | 2402 | CA | CYS | B | 142 | 1187 | 1532 | 1493 | 129 | −465 | −168 | C |
| ATOM | 2403 | C | CYS | B | 142 | 3.128 | −15.708 | −18.415 | 1.00 | 10.41 | | C |
| ANISOU | 2403 | C | CYS | B | 142 | 697 | 1587 | 1672 | −1 | −325 | −248 | C |
| ATOM | 2404 | O | CYS | B | 142 | 2.999 | −16.479 | −17.461 | 1.00 | 11.91 | | O |
| ANISOU | 2404 | O | CYS | B | 142 | 1073 | 1697 | 1756 | −236 | −626 | −115 | O |
| ATOM | 2405 | CB | CYS | B | 142 | 2.282 | −13.362 | −18.229 | 1.00 | 11.41 | | C |
| ANISOU | 2405 | CB | CYS | B | 142 | 1045 | 1731 | 1560 | 153 | −297 | −481 | C |
| ATOM | 2406 | SG | CYS | B | 142 | 2.567 | −11.635 | −17.687 | 1.00 | 11.58 | | S |
| ANISOU | 2406 | SG | CYS | B | 142 | 1419 | 1663 | 1319 | 186 | −108 | −405 | S |
| ATOM | 2407 | N | ARG | B | 143 | 2.972 | −16.067 | −19.703 | 1.00 | 10.07 | | N |
| ANISOU | 2407 | N | ARG | B | 143 | 730 | 1372 | 1725 | 42 | −91 | −189 | N |
| ATOM | 2408 | CA | ARG | B | 143 | 2.610 | −17.453 | −20.000 | 1.00 | 12.00 | | C |
| ANISOU | 2408 | CA | ARG | B | 143 | 1141 | 1468 | 1950 | −29 | −262 | −403 | C |
| ATOM | 2409 | C | ARG | B | 143 | 3.666 | −18.437 | −19.518 | 1.00 | 11.57 | | C |
| ANISOU | 2409 | C | ARG | B | 143 | 1128 | 1318 | 1949 | −18 | −45 | −313 | C |
| ATOM | 2410 | O | ARG | B | 143 | 3.373 | −19.535 | −19.099 | 1.00 | 12.56 | | O |
| ANISOU | 2410 | O | ARG | B | 143 | 1326 | 1655 | 1791 | −45 | −116 | −64 | O |
| ATOM | 2411 | CB | ARG | B | 143 | 2.479 | −17.720 | −21.493 | 1.00 | 13.10 | | C |
| ANISOU | 2411 | CB | ARG | B | 143 | 1369 | 1815 | 1794 | −254 | −98 | −301 | C |
| ATOM | 2412 | CG | ARG | B | 143 | 1.267 | −17.165 | −22.184 | 1.00 | 13.82 | | C |
| ANISOU | 2412 | CG | ARG | B | 143 | 1451 | 2294 | 1505 | 106 | 53 | −346 | C |
| ATOM | 2413 | CD | ARG | B | 143 | 1.057 | −17.824 | −23.553 | 1.00 | 12.78 | | C |
| ANISOU | 2413 | CD | ARG | B | 143 | 1260 | 1952 | 1642 | −56 | −37 | −267 | C |
| ATOM | 2414 | NE | ARG | B | 143 | 2.221 | −17.568 | −24.401 | 1.00 | 12.86 | | N |
| ANISOU | 2414 | NE | ARG | B | 143 | 1341 | 2100 | 1444 | 78 | −28 | −410 | N |
| ATOM | 2415 | CZ | ARG | B | 143 | 2.448 | −16.511 | −25.140 | 1.00 | 10.99 | | C |
| ANISOU | 2415 | CZ | ARG | B | 143 | 753 | 2049 | 1374 | −114 | −215 | −478 | C |
| ATOM | 2416 | NH1 | ARG | B | 143 | 1.584 | −15.516 | −25.187 | 1.00 | 13.22 | | N |
| ANISOU | 2416 | NH1 | ARG | B | 143 | 1157 | 2162 | 1704 | 111 | −364 | −418 | N |
| ATOM | 2417 | NH2 | ARG | B | 143 | 3.555 | −16.411 | −25.863 | 1.00 | 15.13 | | N |
| ANISOU | 2417 | NH2 | ARG | B | 143 | 1015 | 2952 | 1783 | −90 | 72 | −366 | N |
| ATOM | 2418 | N | ASP | B | 144 | 4.917 | −18.034 | −19.637 | 1.00 | 12.10 | | N |
| ANISOU | 2418 | N | ASP | B | 144 | 1090 | 1396 | 2112 | 110 | −137 | −148 | N |
| ATOM | 2419 | CA | ASP | B | 144 | 6.065 | −18.925 | −19.450 | 1.00 | 14.06 | | C |
| ANISOU | 2419 | CA | ASP | B | 144 | 1274 | 1449 | 2620 | 353 | 52 | −91 | C |
| ATOM | 2420 | C | ASP | B | 144 | 6.560 | −18.926 | −18.010 | 1.00 | 14.25 | | C |
| ANISOU | 2420 | C | ASP | B | 144 | 1304 | 1397 | 2715 | 271 | −195 | 165 | C |
| ATOM | 2421 | O | ASP | B | 144 | 7.414 | −19.748 | −17.637 | 1.00 | 19.39 | | O |
| ANISOU | 2421 | O | ASP | B | 144 | 1850 | 1948 | 3568 | 767 | −668 | −171 | O |
| ATOM | 2422 | CB | ASP | B | 144 | 7.200 | −18.556 | −20.388 | 1.00 | 15.44 | | C |
| ANISOU | 2422 | CB | ASP | B | 144 | 1510 | 1612 | 2747 | 483 | 346 | −476 | C |
| ATOM | 2423 | CG | ASP | B | 144 | 6.932 | −18.903 | −21.852 | 1.00 | 16.41 | | C |
| ANISOU | 2423 | CG | ASP | B | 144 | 1612 | 1801 | 2821 | 517 | 500 | −707 | C |
| ATOM | 2424 | OD1 | ASP | B | 144 | 5.961 | −19.643 | −22.105 | 1.00 | 21.93 | | O |
| ANISOU | 2424 | OD1 | ASP | B | 144 | 2011 | 2882 | 3439 | 13 | −158 | −681 | O |
| ATOM | 2425 | OD2 | ASP | B | 144 | 7.744 | −18.403 | −22.669 | 1.00 | 22.34 | | O |
| ANISOU | 2425 | OD2 | ASP | B | 144 | 1982 | 3499 | 3007 | 412 | 1027 | −594 | O |
| ATOM | 2426 | N | ASN | B | 145 | 6.081 | −18.051 | −17.151 | 1.00 | 14.62 | | N |
| ANISOU | 2426 | N | ASN | B | 145 | 1067 | 1828 | 2661 | 313 | −425 | −99 | N |
| ATOM | 2427 | CA | ASN | B | 145 | 6.544 | −17.957 | −15.768 | 1.00 | 14.59 | | C |
| ANISOU | 2427 | CA | ASN | B | 145 | 1047 | 1702 | 2796 | 56 | −511 | −65 | C |
| ATOM | 2428 | C | ASN | B | 145 | 5.443 | −18.077 | −14.741 | 1.00 | 16.32 | | C |
| ANISOU | 2428 | C | ASN | B | 145 | 864 | 2625 | 2714 | −80 | −464 | −950 | C |
| ATOM | 2429 | O | ASN | B | 145 | 5.445 | −17.423 | −13.708 | 1.00 | 23.90 | | O |
| ANISOU | 2429 | O | ASN | B | 145 | 1010 | 3795 | 4276 | −82 | −172 | −2451 | O |
| ATOM | 2430 | CB | ASN | B | 145 | 7.285 | −16.615 | −15.605 | 1.00 | 19.92 | | C |
| ANISOU | 2430 | CB | ASN | B | 145 | 1639 | 2021 | 3907 | −375 | −893 | −89 | C |
| ATOM | 2431 | CG | ASN | B | 145 | 8.601 | −16.679 | −16.359 | 1.00 | 19.25 | | C |
| ANISOU | 2431 | CG | ASN | B | 145 | 1189 | 1720 | 4406 | −147 | −1132 | 873 | C |
| ATOM | 2432 | OD1 | ASN | B | 145 | 9.541 | −17.108 | −15.720 | 1.00 | 27.88 | | O |
| ANISOU | 2432 | OD1 | ASN | B | 145 | 1467 | 3221 | 5904 | −340 | −1673 | 2067 | O |
| ATOM | 2433 | ND2 | ASN | B | 145 | 8.661 | −16.283 | −17.599 | 1.00 | 18.59 | | N |
| ANISOU | 2433 | ND2 | ASN | B | 145 | 1367 | 1501 | 4194 | −12 | −835 | 457 | N |
| ATOM | 2434 | N | GLY | B | 146 | 4.448 | −18.935 | −15.020 | 1.00 | 12.49 | | N |
| ANISOU | 2434 | N | GLY | B | 146 | 1210 | 1557 | 1980 | 42 | −557 | −112 | N |
| ATOM | 2435 | CA | GLY | B | 146 | 3.379 | −19.190 | −14.131 | 1.00 | 13.36 | | C |
| ANISOU | 2435 | CA | GLY | B | 146 | 1383 | 1865 | 1830 | −111 | −579 | −187 | C |
| ATOM | 2436 | C | GLY | B | 146 | 1.999 | −19.034 | −14.704 | 1.00 | 11.05 | | C |
| ANISOU | 2436 | C | GLY | B | 146 | 1282 | 1230 | 1685 | −216 | −468 | −243 | C |
| ATOM | 2437 | O | GLY | B | 146 | 1.023 | −19.329 | −14.015 | 1.00 | 13.94 | | O |
| ANISOU | 2437 | O | GLY | B | 146 | 1351 | 1719 | 2224 | −262 | −430 | 293 | O |
| ATOM | 2438 | N | ASP | B | 147 | 1.893 | −18.572 | −15.934 | 1.00 | 11.71 | | N |
| ANISOU | 2438 | N | ASP | B | 147 | 1309 | 1736 | 1403 | 329 | −309 | −528 | N |
| ATOM | 2439 | CA | ASP | B | 147 | 0.642 | −18.305 | −16.629 | 1.00 | 11.09 | | C |
| ANISOU | 2439 | CA | ASP | B | 147 | 1120 | 1605 | 1490 | −26 | −296 | −389 | C |
| ATOM | 2440 | C | ASP | B | 147 | −0.253 | −17.372 | −15.838 | 1.00 | 9.51 | | C |
| ANISOU | 2440 | C | ASP | B | 147 | 892 | 1703 | 1017 | −173 | −137 | −220 | C |
| ATOM | 2441 | O | ASP | B | 147 | −1.472 | −17.569 | −15.711 | 1.00 | 12.94 | | O |
| ANISOU | 2441 | O | ASP | B | 147 | 891 | 1727 | 2297 | −327 | −129 | −77 | O |
| ATOM | 2442 | CB | ASP | B | 147 | −0.118 | −19.598 | −16.955 | 1.00 | 13.57 | | C |
| ANISOU | 2442 | CB | ASP | B | 147 | 1803 | 1586 | 1765 | −212 | −440 | −346 | C |
| ATOM | 2443 | CG | ASP | B | 147 | −1.226 | −19.356 | −17.970 | 1.00 | 17.07 | | C |
| ANISOU | 2443 | CG | ASP | B | 147 | 1990 | 2439 | 2057 | −149 | −832 | −960 | C |
| ATOM | 2444 | OD1 | ASP | B | 147 | −1.124 | −18.419 | −18.805 | 1.00 | 19.16 | | O |
| ANISOU | 2444 | OD1 | ASP | B | 147 | 2578 | 2348 | 2354 | 503 | −1400 | −749 | O |
| ATOM | 2445 | OD2 | ASP | B | 147 | −2.182 | −20.159 | −17.918 | 1.00 | 23.32 | | O |
| ANISOU | 2445 | OD2 | ASP | B | 147 | 1896 | 3349 | 3616 | −479 | −727 | −1253 | O |
| ATOM | 2446 | N | TYR | B | 148 | 0.313 | −16.299 | −15.329 | 1.00 | 9.97 | | N |
| ANISOU | 2446 | N | TYR | B | 148 | 943 | 1511 | 1336 | −24 | −400 | −249 | N |
| ATOM | 2447 | CA | TYR | B | 148 | −0.447 | −15.343 | −14.554 | 1.00 | 10.32 | | C |
| ANISOU | 2447 | CA | TYR | B | 148 | 1102 | 1423 | 1397 | −225 | −86 | −167 | C |
| ATOM | 2448 | C | TYR | B | 148 | −1.281 | −14.448 | −15.441 | 1.00 | 10.66 | | C |
| ANISOU | 2448 | C | TYR | B | 148 | 1235 | 1328 | 1488 | −32 | −83 | −323 | C |
| ATOM | 2449 | O | TYR | B | 148 | −0.922 | −14.136 | −16.562 | 1.00 | 11.48 | | O |
| ANISOU | 2449 | O | TYR | B | 148 | 1055 | 1738 | 1570 | 159 | −119 | −78 | O |
| ATOM | 2450 | CB | TYR | B | 148 | 0.496 | −14.486 | −13.694 | 1.00 | 15.53 | | C |
| ANISOU | 2450 | CB | TYR | B | 148 | 1295 | 1637 | 2969 | 298 | −775 | −1075 | C |
| ATOM | 2451 | CG | TYR | B | 148 | 1.371 | −15.268 | −12.748 | 1.00 | 17.49 | | C |
| ANISOU | 2451 | CG | TYR | B | 148 | 1851 | 2038 | 2756 | 627 | −1185 | −1680 | C |
| ATOM | 2452 | CD1 | TYR | B | 148 | 0.893 | −16.232 | −11.876 | 1.00 | 20.03 | | C |
| ANISOU | 2452 | CD1 | TYR | B | 148 | 2816 | 2201 | 2593 | 1203 | −1330 | −1201 | C |
| ATOM | 2453 | CD2 | TYR | B | 148 | 2.733 | −15.003 | −12.777 | 1.00 | 22.48 | | C |
| ANISOU | 2453 | CD2 | TYR | B | 148 | 1498 | 3301 | 3743 | 1289 | −1049 | −2272 | C |
| ATOM | 2454 | CE1 | TYR | B | 148 | 1.709 | −16.943 | −11.019 | 1.00 | 23.72 | | C |
| ANISOU | 2454 | CE1 | TYR | B | 148 | 3321 | 3488 | 2203 | 1887 | −1592 | −1582 | C |
| ATOM | 2455 | CE2 | TYR | B | 148 | 3.545 | −15.714 | −11.921 | 1.00 | 25.90 | | C |
| ANISOU | 2455 | CE2 | TYR | B | 148 | 2152 | 4962 | 2727 | 1906 | −1212 | −2703 | C |
| ATOM | 2456 | CZ | TYR | B | 148 | 3.060 | −16.661 | −11.058 | 1.00 | 27.45 | | C |
| ANISOU | 2456 | CZ | TYR | B | 148 | 3183 | 4107 | 3141 | 2121 | −1769 | −2437 | C |
| ATOM | 2457 | OH | TYR | B | 148 | 3.915 | −17.345 | −10.231 | 1.00 | 32.60 | | O |
| ANISOU | 2457 | OH | TYR | B | 148 | 3748 | 5738 | 2900 | 2777 | −2139 | −2642 | O |
| ATOM | 2458 | N | PRO | B | 149 | −2.385 | −13.961 | −14.893 | 1.00 | 10.49 | | N |
| ANISOU | 2458 | N | PRO | B | 149 | 1064 | 1592 | 1331 | −118 | −201 | −176 | N |
| ATOM | 2459 | CA | PRO | B | 149 | −3.144 | −12.940 | −15.613 | 1.00 | 10.04 | | C |
| ANISOU | 2459 | CA | PRO | B | 149 | 1144 | 1275 | 1394 | −144 | −138 | −286 | C |
| ATOM | 2460 | C | PRO | B | 149 | −2.295 | −11.699 | −15.840 | 1.00 | 8.86 | | C |
| ANISOU | 2460 | C | PRO | B | 149 | 894 | 1486 | 986 | −191 | −53 | −338 | C |
| ATOM | 2461 | O | PRO | B | 149 | −1.545 | −11.285 | −14.944 | 1.00 | 9.88 | | O |
| ANISOU | 2461 | O | PRO | B | 149 | 1236 | 1378 | 1139 | −117 | −367 | −422 | O |
| ATOM | 2462 | CB | PRO | B | 149 | −4.324 | −12.644 | −14.710 | 1.00 | 12.00 | | C |
| ANISOU | 2462 | CB | PRO | B | 149 | 1048 | 1289 | 2221 | −198 | 217 | 64 | C |
| ATOM | 2463 | CG | PRO | B | 149 | −4.380 | −13.696 | −13.692 | 1.00 | 13.47 | | C |
| ANISOU | 2463 | CG | PRO | B | 149 | 1298 | 2092 | 1730 | 224 | 57 | 267 | C |
| ATOM | 2464 | CD | PRO | B | 149 | −3.005 | −14.319 | −13.618 | 1.00 | 11.25 | | C |
| ANISOU | 2464 | CD | PRO | B | 149 | 998 | 2198 | 1078 | −70 | −353 | −272 | C |
| ATOM | 2465 | N | TYR | B | 150 | −2.440 | −11.091 | −17.001 | 1.00 | 8.33 | | N |
| ANISOU | 2465 | N | TYR | B | 150 | 718 | 1443 | 1003 | −118 | −92 | −346 | N |
| ATOM | 2466 | CA | TYR | B | 150 | −1.702 | −9.925 | −17.435 | 1.00 | 8.52 | | C |
| ANISOU | 2466 | CA | TYR | B | 150 | 866 | 1274 | 1098 | −33 | 29 | −333 | C |
| ATOM | 2467 | C | TYR | B | 150 | −2.670 | −8.833 | −17.839 | 1.00 | 9.14 | | C |
| ANISOU | 2467 | C | TYR | B | 150 | 1070 | 1307 | 1095 | −86 | −349 | −409 | C |
| ATOM | 2468 | O | TYR | B | 150 | −3.591 | −9.132 | −18.611 | 1.00 | 10.79 | | O |
| ANISOU | 2468 | O | TYR | B | 150 | 1036 | 1825 | 1240 | −192 | −400 | −437 | O |
| ATOM | 2469 | CB | TYR | B | 150 | −0.753 | −10.275 | −18.599 | 1.00 | 10.14 | | C |
| ANISOU | 2469 | CB | TYR | B | 150 | 995 | 1708 | 1151 | −170 | 82 | −430 | C |
| ATOM | 2470 | CG | TYR | B | 150 | −0.051 | −9.070 | −19.185 | 1.00 | 10.40 | | C |
| ANISOU | 2470 | CG | TYR | B | 150 | 1105 | 1958 | 890 | −263 | 175 | −492 | C |
| ATOM | 2471 | CD1 | TYR | B | 150 | 0.751 | −8.280 | −18.364 | 1.00 | 9.32 | | C |
| ANISOU | 2471 | CD1 | TYR | B | 150 | 595 | 1687 | 1259 | −10 | 96 | −486 | C |
| ATOM | 2472 | CD2 | TYR | B | 150 | −0.180 | −8.673 | −20.500 | 1.00 | 11.35 | | C |
| ANISOU | 2472 | CD2 | TYR | B | 150 | 1174 | 1963 | 1174 | −117 | −125 | −207 | C |
| ATOM | 2473 | CE1 | TYR | B | 150 | 1.376 | −7.197 | −18.895 | 1.00 | 10.21 | | C |
| ANISOU | 2473 | CE1 | TYR | B | 150 | 733 | 1733 | 1415 | 40 | 110 | −362 | C |
| ATOM | 2474 | CE2 | TYR | B | 150 | 0.461 | −7.567 | −21.027 | 1.00 | 13.48 | | C |
| ANISOU | 2474 | CE2 | TYR | B | 150 | 1709 | 2041 | 1373 | −281 | −198 | −36 | C |
| ATOM | 2475 | CZ | TYR | B | 150 | 1.254 | −6.809 | −20.193 | 1.00 | 12.59 | | C |
| ANISOU | 2475 | CZ | TYR | B | 150 | 1230 | 2047 | 1507 | −198 | −22 | −168 | C |
| ATOM | 2476 | OH | TYR | B | 150 | 1.911 | −5.696 | −20.672 | 1.00 | 13.89 | | O |
| ANISOU | 2476 | OH | TYR | B | 150 | 1365 | 2172 | 1740 | −272 | 27 | −35 | O |
| ATOM | 2477 | N | PHE | B | 151 | −2.467 | −7.639 | −17.343 | 1.00 | 7.87 | | N |
| ANISOU | 2477 | N | PHE | B | 151 | 761 | 1244 | 984 | −42 | −205 | −286 | N |
| ATOM | 2478 | CA | PHE | B | 151 | −3.211 | −6.451 | −17.686 | 1.00 | 8.92 | | C |
| ANISOU | 2478 | CA | PHE | B | 151 | 843 | 1299 | 1246 | 63 | −12 | −226 | C |
| ATOM | 2479 | C | PHE | B | 151 | −2.303 | −5.308 | −18.130 | 1.00 | 8.68 | | C |
| ANISOU | 2479 | C | PHE | B | 151 | 816 | 1290 | 1193 | 50 | −216 | −100 | C |
| ATOM | 2480 | O | PHE | B | 151 | −1.376 | −4.955 | −17.405 | 1.00 | 10.09 | | O |
| ANISOU | 2480 | O | PHE | B | 151 | 1015 | 1672 | 1146 | −86 | −237 | −166 | O |
| ATOM | 2481 | CB | PHE | B | 151 | −4.054 | −5.967 | −16.503 | 1.00 | 9.35 | | C |
| ANISOU | 2481 | CB | PHE | B | 151 | 1209 | 1131 | 1212 | −28 | 23 | −297 | C |
| ATOM | 2482 | CG | PHE | B | 151 | −5.082 | −6.976 | −16.079 | 1.00 | 8.43 | | C |
| ANISOU | 2482 | CG | PHE | B | 151 | 905 | 1263 | 1034 | 32 | −62 | −304 | C |
| ATOM | 2483 | CD1 | PHE | B | 151 | −4.772 | −7.940 | −15.161 | 1.00 | 8.81 | | C |
| ANISOU | 2483 | CD1 | PHE | B | 151 | 1042 | 1418 | 889 | 44 | 85 | −270 | C |
| ATOM | 2484 | CD2 | PHE | B | 151 | −6.350 | −6.993 | −16.644 | 1.00 | 11.59 | | C |
| ANISOU | 2484 | CD2 | PHE | B | 151 | 921 | 2182 | 1299 | 3 | −87 | −198 | C |
| ATOM | 2485 | CE1 | PHE | B | 151 | −5.675 | −8.897 | −14.775 | 1.00 | 9.88 | | C |
| ANISOU | 2485 | CE1 | PHE | B | 151 | 989 | 1527 | 1238 | −21 | −20 | −51 | C |
| ATOM | 2486 | CE2 | PHE | B | 151 | −7.264 | −7.929 | −16.241 | 1.00 | 10.64 | | C |
| ANISOU | 2486 | CE2 | PHE | B | 151 | 716 | 1908 | 1421 | 138 | −83 | −348 | C |
| ATOM | 2487 | CZ | PHE | B | 151 | −6.940 | −8.901 | −15.352 | 1.00 | 10.93 | | C |
| ANISOU | 2487 | CZ | PHE | B | 151 | 1150 | 1656 | 1345 | 6 | −233 | −492 | C |
| ATOM | 2488 | N | GLU | B | 152 | −2.640 | −4.730 | −19.260 | 1.00 | 10.20 | | N |
| ANISOU | 2488 | N | GLU | B | 152 | 1130 | 1610 | 1135 | −49 | −242 | −63 | N |
| ATOM | 2489 | CA | GLU | B | 152 | −2.032 | −3.490 | −19.735 | 1.00 | 11.45 | | C |
| ANISOU | 2489 | CA | GLU | B | 152 | 1318 | 1698 | 1336 | 125 | −296 | 231 | C |
| ATOM | 2490 | C | GLU | B | 152 | −2.849 | −2.332 | −19.194 | 1.00 | 10.72 | | C |
| ANISOU | 2490 | C | GLU | B | 152 | 986 | 1668 | 1419 | −12 | −253 | 119 | C |
| ATOM | 2491 | O | GLU | B | 152 | −4.042 | −2.199 | −19.519 | 1.00 | 14.12 | | O |
| ANISOU | 2491 | O | GLU | B | 152 | 1016 | 2171 | 2179 | 29 | −491 | −84 | O |
| ATOM | 2492 | CB | GLU | B | 152 | −2.012 | −3.545 | −21.261 | 1.00 | 14.11 | | C |
| ANISOU | 2492 | CB | GLU | B | 152 | 1719 | 2329 | 1315 | 148 | 109 | 208 | C |
| ATOM | 2493 | CG | GLU | B | 152 | −0.947 | −4.524 | −21.719 | 1.00 | 18.49 | | C |
| ANISOU | 2493 | CG | GLU | B | 152 | 2410 | 2296 | 2321 | 130 | 967 | 127 | C |
| ATOM | 2494 | CD | GLU | B | 152 | −0.086 | −4.168 | −22.891 | 1.00 | 14.54 | | C |
| ANISOU | 2494 | CD | GLU | B | 152 | 1630 | 2321 | 1573 | −353 | 209 | −232 | C |
| ATOM | 2495 | OE1 | GLU | B | 152 | −0.469 | −3.347 | −23.733 | 1.00 | 19.19 | | O |
| ANISOU | 2495 | OE1 | GLU | B | 152 | 2378 | 2951 | 1964 | −253 | 81 | 273 | O |
| ATOM | 2496 | OE2 | GLU | B | 152 | 1.004 | −4.779 | −22.937 | 1.00 | 18.15 | | O |
| ANISOU | 2496 | OE2 | GLU | B | 152 | 2068 | 2726 | 2101 | 59 | 568 | −207 | O |
| ATOM | 2497 | N | THR | B | 153 | −2.267 | −1.569 | −18.302 | 1.00 | 10.97 | | N |
| ANISOU | 2497 | N | THR | B | 153 | 920 | 1758 | 1492 | 78 | −284 | 83 | N |
| ATOM | 2498 | CA | THR | B | 153 | −2.954 | −0.578 | −17.527 | 1.00 | 11.24 | | C |
| ANISOU | 2498 | CA | THR | B | 153 | 1031 | 1743 | 1498 | 2 | −148 | 80 | C |
| ATOM | 2499 | C | THR | B | 153 | −2.463 | 0.838 | −17.792 | 1.00 | 11.93 | | C |
| ANISOU | 2499 | C | THR | B | 153 | 1179 | 1702 | 1650 | −50 | −314 | 59 | C |
| ATOM | 2500 | O | THR | B | 153 | −1.370 | 1.064 | −18.272 | 1.00 | 12.52 | | O |
| ANISOU | 2500 | O | THR | B | 153 | 1256 | 1866 | 1635 | −252 | −390 | 308 | O |
| ATOM | 2501 | CB | THR | B | 153 | −2.872 | −0.843 | −16.012 | 1.00 | 11.20 | | C |
| ANISOU | 2501 | CB | THR | B | 153 | 960 | 1820 | 1477 | −58 | −265 | 10 | C |
| ATOM | 2502 | OG1 | THR | B | 153 | −1.523 | −0.730 | −15.551 | 1.00 | 12.13 | | O |
| ANISOU | 2502 | OG1 | THR | B | 153 | 1035 | 1663 | 1912 | −214 | −468 | 223 | O |
| ATOM | 2503 | CG2 | THR | B | 153 | −3.301 | −2.243 | −15.649 | 1.00 | 12.61 | | C |
| ANISOU | 2503 | CG2 | THR | B | 153 | 1233 | 2098 | 1462 | −522 | −414 | 123 | C |
| ATOM | 2504 | N | SER | B | 154 | −3.357 | 1.761 | −17.461 | 1.00 | 13.96 | | N |
| ANISOU | 2504 | N | SER | B | 154 | 1565 | 1853 | 1887 | 236 | −640 | −294 | N |
| ATOM | 2505 | CA | SER | B | 154 | −2.995 | 3.163 | −17.399 | 1.00 | 12.54 | | C |
| ANISOU | 2505 | CA | SER | B | 154 | 1071 | 1858 | 1835 | 203 | −223 | −34 | C |
| ATOM | 2506 | C | SER | B | 154 | −3.616 | 3.789 | −16.174 | 1.00 | 11.38 | | C |
| ANISOU | 2506 | C | SER | B | 154 | 1146 | 1479 | 1699 | 286 | −362 | 109 | C |
| ATOM | 2507 | O | SER | B | 154 | −4.841 | 3.922 | −16.105 | 1.00 | 14.94 | | O |
| ANISOU | 2507 | O | SER | B | 154 | 1186 | 2127 | 2364 | 399 | −237 | −404 | O |
| ATOM | 2508 | CB | SER | B | 154 | −3.431 | 3.929 | −18.644 | 1.00 | 15.03 | | C |
| ANISOU | 2508 | CB | SER | B | 154 | 1802 | 2218 | 1689 | 185 | −157 | 24 | C |
| ATOM | 2509 | OG | SER | B | 154 | −3.132 | 5.326 | −18.482 | 1.00 | 16.25 | | O |
| ANISOU | 2509 | OG | SER | B | 154 | 1674 | 2169 | 2333 | 152 | −483 | 378 | O |
| ATOM | 2510 | N | ALA | B | 155 | −2.812 | 4.189 | −15.206 | 1.00 | 13.31 | | N |
| ANISOU | 2510 | N | ALA | B | 155 | 1522 | 1493 | 2044 | 236 | −516 | −184 | N |
| ATOM | 2511 | CA | ALA | B | 155 | −3.411 | 4.919 | −14.083 | 1.00 | 12.96 | | C |
| ANISOU | 2511 | CA | ALA | B | 155 | 1434 | 1422 | 2068 | −32 | −320 | −177 | C |
| ATOM | 2512 | C | ALA | B | 155 | −3.921 | 6.273 | −14.529 | 1.00 | 14.17 | | C |
| ANISOU | 2512 | C | ALA | B | 155 | 1759 | 1516 | 2108 | 146 | −158 | −175 | C |
| ATOM | 2513 | O | ALA | B | 155 | −4.862 | 6.842 | −13.970 | 1.00 | 15.80 | | O |
| ANISOU | 2513 | O | ALA | B | 155 | 2056 | 1666 | 2283 | 400 | 230 | 350 | O |
| ATOM | 2514 | CB | ALA | B | 155 | −2.398 | 5.091 | −12.990 | 1.00 | 14.79 | | C |
| ANISOU | 2514 | CB | ALA | B | 155 | 1736 | 1878 | 2005 | −21 | −390 | −269 | C |
| ATOM | 2515 | N | LYS | B | 156 | −3.228 | 6.799 | −15.552 | 1.00 | 13.74 | | N |
| ANISOU | 2515 | N | LYS | B | 156 | 1615 | 1636 | 1969 | 363 | −182 | −68 | N |
| ATOM | 2516 | CA | LYS | B | 156 | −3.631 | 8.116 | −16.042 | 1.00 | 15.83 | | C |
| ANISOU | 2516 | CA | LYS | B | 156 | 2102 | 1542 | 2369 | 408 | −241 | −20 | C |
| ATOM | 2517 | C | LYS | B | 156 | −5.012 | 8.108 | −16.651 | 1.00 | 17.28 | | C |
| ANISOU | 2517 | C | LYS | B | 156 | 2176 | 1660 | 2728 | 479 | −457 | 252 | C |
| ATOM | 2518 | O | LYS | B | 156 | −5.816 | 9.045 | −16.436 | 1.00 | 21.21 | | O |
| ANISOU | 2518 | O | LYS | B | 156 | 2359 | 2801 | 2901 | 1067 | −314 | −310 | O |
| ATOM | 2519 | CB | LYS | B | 156 | −2.590 | 8.611 | −17.047 | 1.00 | 16.63 | | C |
| ANISOU | 2519 | CB | LYS | B | 156 | 2365 | 1434 | 2517 | 161 | −165 | −29 | C |
| ATOM | 2520 | CG | LYS | B | 156 | −2.827 | 10.010 | −17.606 | 1.00 | 22.95 | | C |
| ANISOU | 2520 | CG | LYS | B | 156 | 3775 | 2041 | 2905 | 94 | −854 | 765 | C |
| ATOM | 2521 | CD | LYS | B | 156 | −1.611 | 10.593 | −18.292 | 1.00 | 32.28 | | C |
| ANISOU | 2521 | CD | LYS | B | 156 | 5469 | 3061 | 3735 | −909 | −71 | 1414 | C |
| ATOM | 2522 | CE | LYS | B | 156 | −1.687 | 12.107 | −18.486 | 1.00 | 37.35 | | C |
| ANISOU | 2522 | CE | LYS | B | 156 | 5647 | 3182 | 5363 | −604 | 41 | 1824 | C |
| ATOM | 2523 | NZ | LYS | B | 156 | −3.078 | 12.559 | −18.733 | 1.00 | 55.77 | | N |
| ANISOU | 2523 | NZ | LYS | B | 156 | 6637 | 5631 | 8920 | 1747 | 235 | 478 | N |
| ATOM | 2524 | N | ASP | B | 157 | −5.370 | 7.096 | −17.428 | 1.00 | 20.15 | | N |
| ANISOU | 2524 | N | ASP | B | 157 | 2707 | 1532 | 3417 | 360 | −1075 | 308 | N |
| ATOM | 2525 | CA | ASP | B | 157 | −6.700 | 7.140 | −18.049 | 1.00 | 22.07 | | C |
| ANISOU | 2525 | CA | ASP | B | 157 | 2915 | 1837 | 3633 | 561 | −1357 | 182 | C |
| ATOM | 2526 | C | ASP | B | 157 | −7.638 | 6.071 | −17.531 | 1.00 | 21.03 | | C |
| ANISOU | 2526 | C | ASP | B | 157 | 2524 | 1728 | 3737 | 607 | −1501 | −15 | C |
| ATOM | 2527 | O | ASP | B | 157 | −8.773 | 5.985 | −18.028 | 1.00 | 24.17 | | O |
| ANISOU | 2527 | O | ASP | B | 157 | 2271 | 3117 | 3794 | 788 | −1266 | 248 | O |
| ATOM | 2528 | CB | ASP | B | 157 | −6.592 | 7.105 | −19.556 | 1.00 | 26.85 | | C |
| ANISOU | 2528 | CB | ASP | B | 157 | 3374 | 3182 | 3645 | −92 | −1469 | 49 | C |
| ATOM | 2529 | CG | ASP | B | 157 | −6.083 | 5.861 | −20.219 | 1.00 | 25.42 | | C |
| ANISOU | 2529 | CG | ASP | B | 157 | 3341 | 3418 | 2902 | 98 | −989 | 367 | C |
| ATOM | 2530 | OD1 | ASP | B | 157 | −6.240 | 4.754 | −19.676 | 1.00 | 30.99 | | O |
| ANISOU | 2530 | OD1 | ASP | B | 157 | 4611 | 3400 | 3766 | 1032 | −1562 | 928 | O |
| ATOM | 2531 | OD2 | ASP | B | 157 | −5.506 | 6.064 | −21.331 | 1.00 | 40.27 | | O |
| ANISOU | 2531 | OD2 | ASP | B | 157 | 6585 | 6251 | 2467 | 1164 | −498 | 941 | O |
| ATOM | 2532 | N | ALA | B | 158 | −7.223 | 5.264 | −16.568 | 1.00 | 18.04 | | N |
| ANISOU | 2532 | N | ALA | B | 158 | 1922 | 1980 | 2952 | 436 | −965 | −183 | N |
| ATOM | 2533 | CA | ALA | B | 158 | −7.962 | 4.278 | −15.812 | 1.00 | 17.34 | | C |
| ANISOU | 2533 | CA | ALA | B | 158 | 1872 | 2411 | 2303 | 360 | −559 | −534 | C |
| ATOM | 2534 | C | ALA | B | 158 | −8.079 | 2.934 | −16.544 | 1.00 | 14.99 | | C |
| ANISOU | 2534 | C | ALA | B | 158 | 1400 | 2135 | 2161 | 302 | −519 | −212 | C |
| ATOM | 2535 | O | ALA | B | 158 | −8.639 | 1.976 | −16.019 | 1.00 | 18.25 | | O |
| ANISOU | 2535 | O | ALA | B | 158 | 1902 | 2412 | 2622 | 99 | −560 | 131 | O |
| ATOM | 2536 | CB | ALA | B | 158 | −9.330 | 4.796 | −15.420 | 1.00 | 21.22 | | C |
| ANISOU | 2536 | CB | ALA | B | 158 | 1617 | 2581 | 3865 | 140 | −800 | −1481 | C |
| ATOM | 2537 | N | THR | B | 159 | −7.513 | 2.811 | −17.720 | 1.00 | 15.89 | | N |
| ANISOU | 2537 | N | THR | B | 159 | 1799 | 2140 | 2100 | 314 | −560 | −280 | N |
| ATOM | 2538 | CA | THR | B | 159 | −7.591 | 1.565 | −18.486 | 1.00 | 14.00 | | C |
| ANISOU | 2538 | CA | THR | B | 159 | 1213 | 2130 | 1978 | −20 | −407 | −154 | C |
| ATOM | 2539 | C | THR | B | 159 | −7.150 | 0.339 | −17.696 | 1.00 | 12.84 | | C |
| ANISOU | 2539 | C | THR | B | 159 | 1155 | 2060 | 1662 | −11 | −369 | −297 | C |
| ATOM | 2540 | O | THR | B | 159 | −6.021 | 0.281 | −17.223 | 1.00 | 12.94 | | O |
| ANISOU | 2540 | O | THR | B | 159 | 885 | 2216 | 1816 | 149 | −159 | −430 | O |
| ATOM | 2541 | CB | THR | B | 159 | −6.720 | 1.655 | −19.750 | 1.00 | 16.68 | | C |
| ANISOU | 2541 | CB | THR | B | 159 | 1887 | 2695 | 1753 | 94 | −295 | −14 | C |
| ATOM | 2542 | OG1 | THR | B | 159 | −7.138 | 2.742 | −20.562 | 1.00 | 21.35 | | O |
| ANISOU | 2542 | OG1 | THR | B | 159 | 2353 | 3307 | 2454 | 215 | −223 | 651 | O |
| ATOM | 2543 | CG2 | THR | B | 159 | −6.872 | 0.420 | −20.615 | 1.00 | 18.79 | | C |
| ANISOU | 2543 | CG2 | THR | B | 159 | 1634 | 3337 | 2171 | −55 | −237 | −567 | C |
| ATOM | 2544 | N | ASN | B | 160 | −8.055 | −0.625 | −17.570 | 1.00 | 11.97 | | N |
| ANISOU | 2544 | N | ASN | B | 160 | 1017 | 2203 | 1327 | −1 | −262 | −268 | N |
| ATOM | 2545 | CA | ASN | B | 160 | −7.902 | −1.901 | −16.920 | 1.00 | 12.21 | | C |
| ANISOU | 2545 | CA | ASN | B | 160 | 1159 | 2210 | 1270 | −43 | −107 | −273 | C |
| ATOM | 2546 | C | ASN | B | 160 | −7.497 | −1.832 | −15.457 | 1.00 | 11.50 | | C |
| ANISOU | 2546 | C | ASN | B | 160 | 975 | 1987 | 1408 | −73 | −311 | −236 | C |
| ATOM | 2547 | O | ASN | B | 160 | −7.155 | −2.876 | −14.907 | 1.00 | 13.93 | | O |
| ANISOU | 2547 | O | ASN | B | 160 | 1254 | 2337 | 1701 | 261 | −287 | −64 | O |
| ATOM | 2548 | CB | ASN | B | 160 | −6.840 | −2.723 | −17.653 | 1.00 | 14.02 | | C |
| ANISOU | 2548 | CB | ASN | B | 160 | 1470 | 2060 | 1796 | −16 | −6 | −509 | C |
| ATOM | 2549 | CG | ASN | B | 160 | −7.363 | −3.288 | −18.931 | 1.00 | 13.50 | | C |
| ANISOU | 2549 | CG | ASN | B | 160 | 997 | 2404 | 1729 | −132 | 166 | −575 | C |
| ATOM | 2550 | OD1 | ASN | B | 160 | −8.568 | −3.534 | −19.069 | 1.00 | 22.65 | | O |
| ANISOU | 2550 | OD1 | ASN | B | 160 | 1155 | 4019 | 3433 | −838 | 235 | −1544 | O |
| ATOM | 2551 | ND2 | ASN | B | 160 | −6.454 | −3.534 | −19.831 | 1.00 | 18.60 | | N |
| ANISOU | 2551 | ND2 | ASN | B | 160 | 1516 | 3313 | 2239 | 77 | 547 | −991 | N |
| ATOM | 2552 | N | VAL | B | 161 | −7.569 | −0.668 | −14.844 | 1.00 | 12.70 | | N |
| ANISOU | 2552 | N | VAL | B | 161 | 1030 | 2283 | 1513 | 26 | −229 | −553 | N |
| ATOM | 2553 | CA | VAL | B | 161 | −7.110 | −0.635 | −13.444 | 1.00 | 12.69 | | C |
| ANISOU | 2553 | CA | VAL | B | 161 | 1413 | 1853 | 1556 | −72 | −305 | −369 | C |
| ATOM | 2554 | C | VAL | B | 161 | −8.048 | −1.414 | −12.553 | 1.00 | 12.87 | | C |
| ANISOU | 2554 | C | VAL | B | 161 | 1276 | 1990 | 1623 | −129 | −526 | −219 | C |
| ATOM | 2555 | O | VAL | B | 161 | −7.616 | −2.278 | −11.747 | 1.00 | 13.95 | | O |
| ANISOU | 2555 | O | VAL | B | 161 | 1531 | 2399 | 1371 | 217 | −227 | −138 | O |
| ATOM | 2556 | CB | VAL | B | 161 | −6.967 | 0.816 | −12.969 | 1.00 | 11.39 | | C |
| ANISOU | 2556 | CB | VAL | B | 161 | 1112 | 1795 | 1420 | 181 | −267 | −318 | C |
| ATOM | 2557 | CG1 | VAL | B | 161 | −6.628 | 0.923 | −11.495 | 1.00 | 17.02 | | C |
| ANISOU | 2557 | CG1 | VAL | B | 161 | 2766 | 2370 | 1333 | −834 | −116 | −350 | C |
| ATOM | 2558 | CG2 | VAL | B | 161 | −5.864 | 1.460 | −13.805 | 1.00 | 13.38 | | C |
| ANISOU | 2558 | CG2 | VAL | B | 161 | 1685 | 2008 | 1390 | −257 | −320 | −79 | C |
| ATOM | 2559 | N | ALA | B | 162 | −9.362 | −1.164 | −12.613 | 1.00 | 12.83 | | N |
| ANISOU | 2559 | N | ALA | B | 162 | 1351 | 2059 | 1465 | −1 | −340 | −362 | N |
| ATOM | 2560 | CA | ALA | B | 162 | −10.251 | −1.876 | −11.713 | 1.00 | 12.44 | | C |
| ANISOU | 2560 | CA | ALA | B | 162 | 1402 | 2002 | 1323 | 314 | −172 | −330 | C |
| ATOM | 2561 | C | ALA | B | 162 | −10.244 | −3.359 | −12.043 | 1.00 | 11.90 | | C |
| ANISOU | 2561 | C | ALA | B | 162 | 1313 | 1961 | 1248 | 143 | −520 | −266 | C |
| ATOM | 2562 | O | ALA | B | 162 | −10.271 | −4.203 | −11.145 | 1.00 | 11.98 | | O |
| ANISOU | 2562 | O | ALA | B | 162 | 1220 | 2035 | 1297 | 41 | −84 | −212 | O |
| ATOM | 2563 | CB | ALA | B | 162 | −11.669 | −1.353 | −11.822 | 1.00 | 14.38 | | C |
| ANISOU | 2563 | CB | ALA | B | 162 | 1325 | 2144 | 1994 | 258 | −321 | −247 | C |
| ATOM | 2564 | N | ALA | B | 163 | −10.167 | −3.673 | −13.339 | 1.00 | 11.29 | | N |
| ANISOU | 2564 | N | ALA | B | 163 | 1100 | 1919 | 1270 | −110 | −162 | −228 | N |
| ATOM | 2565 | CA | ALA | B | 163 | −10.162 | −5.060 | −13.780 | 1.00 | 12.11 | | C |
| ANISOU | 2565 | CA | ALA | B | 163 | 1146 | 2110 | 1344 | 115 | −173 | −411 | C |
| ATOM | 2566 | C | ALA | B | 163 | −8.995 | −5.835 | −13.169 | 1.00 | 11.11 | | C |
| ANISOU | 2566 | C | ALA | B | 163 | 1117 | 2085 | 1018 | 104 | 8 | −333 | C |
| ATOM | 2567 | O | ALA | B | 163 | −9.156 | −7.001 | −12.797 | 1.00 | 11.86 | | O |
| ANISOU | 2567 | O | ALA | B | 163 | 1251 | 2100 | 1156 | 32 | 8 | −283 | O |
| ATOM | 2568 | CB | ALA | B | 163 | −10.154 | −5.190 | −15.284 | 1.00 | 14.09 | | C |
| ANISOU | 2568 | CB | ALA | B | 163 | 1723 | 2332 | 1300 | 140 | −421 | −382 | C |
| ATOM | 2569 | N | ALA | B | 164 | −7.823 | −5.218 | −13.065 | 1.00 | 10.59 | | N |
| ANISOU | 2569 | N | ALA | B | 164 | 1044 | 1847 | 1131 | 233 | −14 | −325 | N |
| ATOM | 2570 | CA | ALA | B | 164 | −6.672 | −5.897 | −12.501 | 1.00 | 10.26 | | C |
| ANISOU | 2570 | CA | ALA | B | 164 | 1113 | 1448 | 1339 | 61 | −99 | −266 | C |
| ATOM | 2571 | C | ALA | B | 164 | −6.866 | −6.216 | −11.031 | 1.00 | 9.74 | | C |
| ANISOU | 2571 | C | ALA | B | 164 | 867 | 1551 | 1284 | −150 | −192 | −298 | C |
| ATOM | 2572 | O | ALA | B | 164 | −6.553 | −7.296 | −10.572 | 1.00 | 10.87 | | O |
| ANISOU | 2572 | O | ALA | B | 164 | 1197 | 1614 | 1321 | 5 | −151 | −275 | O |
| ATOM | 2573 | CB | ALA | B | 164 | −5.443 | −5.009 | −12.702 | 1.00 | 12.21 | | C |
| ANISOU | 2573 | CB | ALA | B | 164 | 1023 | 2186 | 1432 | −44 | 251 | −343 | C |
| ATOM | 2574 | N | PHE | B | 165 | −7.348 | −5.264 | −10.255 | 1.00 | 10.66 | | N |
| ANISOU | 2574 | N | PHE | B | 165 | 1129 | 1569 | 1351 | 18 | −215 | −266 | N |
| ATOM | 2575 | CA | PHE | B | 165 | −7.574 | −5.479 | −8.836 | 1.00 | 11.38 | | C |
| ANISOU | 2575 | CA | PHE | B | 165 | 1055 | 1961 | 1310 | 295 | −207 | −412 | C |
| ATOM | 2576 | C | PHE | B | 165 | −8.709 | −6.456 | −8.605 | 1.00 | 11.42 | | C |
| ANISOU | 2576 | C | PHE | B | 165 | 1240 | 2209 | 890 | 111 | −180 | −281 | C |
| ATOM | 2577 | O | PHE | B | 165 | −8.619 | −7.280 | −7.693 | 1.00 | 12.92 | | O |
| ANISOU | 2577 | O | PHE | B | 165 | 1600 | 2243 | 1066 | 160 | −342 | −180 | O |
| ATOM | 2578 | CB | PHE | B | 165 | −7.827 | −4.156 | −8.115 | 1.00 | 13.43 | | C |
| ANISOU | 2578 | CB | PHE | B | 165 | 1275 | 2145 | 1684 | 370 | −221 | −660 | C |
| ATOM | 2579 | CG | PHE | B | 165 | −6.574 | −3.316 | −7.953 | 1.00 | 12.20 | | C |
| ANISOU | 2579 | CG | PHE | B | 165 | 1360 | 2058 | 1215 | 386 | −265 | −648 | C |
| ATOM | 2580 | CD1 | PHE | B | 165 | −5.697 | −3.678 | −6.942 | 1.00 | 13.51 | | C |
| ANISOU | 2580 | CD1 | PHE | B | 165 | 1296 | 2296 | 1540 | 348 | −330 | −345 | C |
| ATOM | 2581 | CD2 | PHE | B | 165 | −6.269 | −2.246 | −8.737 | 1.00 | 12.99 | | C |
| ANISOU | 2581 | CD2 | PHE | B | 165 | 1265 | 1925 | 1746 | 438 | −595 | −492 | C |
| ATOM | 2582 | CB1 | PHE | B | 165 | −4.552 | −2.926 | −6.792 | 1.00 | 15.79 | | C |
| ANISOU | 2582 | CB1 | PHE | B | 165 | 1593 | 2789 | 1616 | −45 | −472 | −271 | C |
| ATOM | 2583 | CB2 | PHE | B | 165 | −5.124 | −1.488 | −8.595 | 1.00 | 14.93 | | C |
| ANISOU | 2583 | CB2 | PHE | B | 165 | 1469 | 2418 | 1787 | 86 | −624 | −392 | C |
| ATOM | 2584 | CZ | PHE | B | 165 | −4.260 | −1.829 | −7.578 | 1.00 | 14.70 | | C |
| ANISOU | 2584 | CZ | PHE | B | 165 | 1476 | 2607 | 1501 | −11 | −549 | −409 | C |
| ATOM | 2585 | N | GLU | B | 166 | −9.753 | −6.368 | −9.451 | 1.00 | 11.68 | | N |
| ANISOU | 2585 | N | GLU | B | 166 | 971 | 2160 | 1305 | 289 | −180 | −170 | N |
| ATOM | 2586 | CA | GLU | B | 166 | −10.841 | −7.338 | −9.295 | 1.00 | 11.81 | | C |
| ANISOU | 2586 | CA | GLU | B | 166 | 1073 | 2218 | 1195 | 185 | −39 | −319 | C |
| ATOM | 2587 | C | GLU | B | 166 | −10.370 | −8.754 | −9.600 | 1.00 | 11.03 | | C |
| ANISOU | 2587 | C | GLU | B | 166 | 678 | 2223 | 1291 | 172 | 13 | −242 | C |
| ATOM | 2588 | O | GLU | B | 166 | −10.793 | −9.740 | −8.991 | 1.00 | 12.74 | | O |
| ANISOU | 2588 | O | GLU | B | 166 | 1111 | 2234 | 1497 | 223 | 207 | −95 | O |
| ATOM | 2589 | CB | GLU | B | 166 | −12.055 | −6.949 | −10.160 | 1.00 | 14.77 | | C |
| ANISOU | 2589 | CB | GLU | B | 166 | 1018 | 2454 | 2139 | 446 | −414 | −682 | C |
| ATOM | 2590 | CG | GLU | B | 166 | −12.639 | −5.623 | −9.764 | 1.00 | 15.57 | | C |
| ANISOU | 2590 | CG | GLU | B | 166 | 1068 | 2136 | 2710 | 204 | 372 | −263 | C |
| ATOM | 2591 | CD | GLU | B | 166 | −13.483 | −4.842 | −10.727 | 1.00 | 22.26 | | C |
| ANISOU | 2591 | CD | GLU | B | 166 | 2566 | 2616 | 3275 | 949 | −345 | −369 | C |
| ATOM | 2592 | OE1 | GLU | B | 166 | −13.323 | −4.905 | −11.986 | 1.00 | 30.74 | | O |
| ANISOU | 2592 | OE1 | GLU | B | 166 | 2755 | 5793 | 3131 | 169 | −384 | 356 | O |
| ATOM | 2593 | OE2 | GLU | B | 166 | −14.348 | −4.100 | −10.204 | 1.00 | 32.41 | | O |
| ANISOU | 2593 | OE2 | GLU | B | 166 | 2770 | 4227 | 5318 | 1973 | −1120 | −1601 | O |
| ATOM | 2594 | N | GLU | B | 167 | −9.464 | −8.917 | −10.556 | 1.00 | 10.19 | | N |
| ANISOU | 2594 | N | GLU | B | 167 | 731 | 1945 | 1198 | 7 | −66 | −266 | N |
| ATOM | 2595 | CA | GLU | B | 167 | −8.888 | −10.226 | −10.884 | 1.00 | 10.46 | | C |
| ANISOU | 2595 | CA | GLU | B | 167 | 982 | 2083 | 908 | 146 | −5 | −256 | C |
| ATOM | 2596 | C | GLU | B | 167 | −8.088 | −10.740 | −9.700 | 1.00 | 11.23 | | C |
| ANISOU | 2596 | C | GLU | B | 167 | 1268 | 1980 | 1021 | 280 | −97 | −324 | C |
| ATOM | 2597 | O | GLU | B | 167 | −8.151 | −11.942 | −9.437 | 1.00 | 11.62 | | O |
| ANISOU | 2597 | O | GLU | B | 167 | 1259 | 1896 | 1260 | 274 | −84 | −295 | O |
| ATOM | 2598 | CB | GLU | B | 167 | −8.046 | −10.169 | −12.151 | 1.00 | 10.95 | | C |
| ANISOU | 2598 | CB | GLU | B | 167 | 902 | 2046 | 1212 | 120 | 135 | 19 | C |
| ATOM | 2599 | CG | GLU | B | 167 | −7.420 | −11.514 | −12.476 | 1.00 | 13.35 | | C |
| ANISOU | 2599 | CG | GLU | B | 167 | 1235 | 2339 | 1498 | 121 | −48 | −941 | C |
| ATOM | 2600 | CD | GLU | B | 167 | −8.346 | −12.653 | −12.843 | 1.00 | 15.35 | | C |
| ANISOU | 2600 | CD | GLU | B | 167 | 1702 | 2778 | 1350 | −206 | −157 | −1146 | C |
| ATOM | 2601 | OE1 | GLU | B | 167 | −9.566 | −12.505 | −12.808 | 1.00 | 21.40 | | O |
| ANISOU | 2601 | OE1 | GLU | B | 167 | 1623 | 3650 | 2859 | −461 | −294 | −1589 | O |
| ATOM | 2602 | OE2 | GLU | B | 167 | −7.822 | −13.733 | −13.175 | 1.00 | 23.59 | | O |
| ANISOU | 2602 | OE2 | GLU | B | 167 | 2665 | 2518 | 3778 | 184 | −1326 | −1386 | O |
| ATOM | 2603 | N | ALA | B | 168 | −7.439 | −9.816 | −8.988 | 1.00 | 11.57 | | N |
| ANISOU | 2603 | N | ALA | B | 168 | 1150 | 2017 | 1229 | 59 | −211 | −140 | N |
| ATOM | 2604 | CA | ALA | B | 168 | −6.644 | −10.290 | −7.862 | 1.00 | 12.16 | | C |
| ANISOU | 2604 | CA | ALA | B | 168 | 1093 | 2337 | 1192 | 319 | −196 | −426 | C |
| ATOM | 2605 | C | ALA | B | 168 | −7.526 | −11.009 | −6.858 | 1.00 | 12.37 | | C |
| ANISOU | 2605 | C | ALA | B | 168 | 1327 | 2497 | 877 | 389 | −329 | −298 | C |
| ATOM | 2606 | O | ALA | B | 168 | −7.231 | −12.070 | −6.344 | 1.00 | 14.18 | | O |
| ANISOU | 2606 | O | ALA | B | 168 | 1546 | 2216 | 1626 | 7 | −670 | −234 | O |
| ATOM | 2607 | CB | ALA | B | 168 | −5.916 | −9.149 | −7.169 | 1.00 | 15.47 | | C |
| ANISOU | 2607 | CB | ALA | B | 168 | 1665 | 2452 | 1762 | 207 | −692 | −464 | C |
| ATOM | 2608 | N | VAL | B | 169 | −8.683 | −10.406 | −6.577 | 1.00 | 13.80 | | N |
| ANISOU | 2608 | N | VAL | B | 169 | 1112 | 2431 | 1700 | 46 | 9 | −507 | N |
| ATOM | 2609 | CA | VAL | B | 169 | −9.527 | −11.061 | −5.584 | 1.00 | 16.44 | | C |
| ANISOU | 2609 | CA | VAL | B | 169 | 1459 | 3012 | 1776 | −200 | 24 | −404 | C |
| ATOM | 2610 | C | VAL | B | 169 | −10.062 | −12.391 | −6.105 | 1.00 | 15.56 | | C |
| ANISOU | 2610 | C | VAL | B | 169 | 1616 | 2983 | 1314 | −387 | −206 | −34 | C |
| ATOM | 2611 | O | VAL | B | 169 | −10.154 | −13.409 | −5.397 | 1.00 | 14.64 | | O |
| ANISOU | 2611 | O | VAL | B | 169 | 1109 | 3217 | 1238 | −384 | −85 | 91 | O |
| ATOM | 2612 | CB | VAL | B | 169 | −10.680 | −10.114 | −5.193 | 1.00 | 19.07 | | C |
| ANISOU | 2612 | CB | VAL | B | 169 | 1544 | 3667 | 2034 | −80 | 500 | −600 | C |
| ATOM | 2613 | CG1 | VAL | B | 169 | −11.669 | −10.909 | −4.356 | 1.00 | 26.39 | | C |
| ANISOU | 2613 | CG1 | VAL | B | 169 | 3172 | 4185 | 2671 | −422 | 1665 | −660 | C |
| ATOM | 2614 | CG2 | VAL | B | 169 | −10.142 | −8.890 | −4.503 | 1.00 | 23.16 | | C |
| ANISOU | 2614 | CG2 | VAL | B | 169 | 3147 | 3033 | 2622 | 235 | 138 | −506 | C |
| ATOM | 2615 | N | ARG | B | 170 | −10.426 | −12.428 | −7.392 | 1.00 | 14.23 | | N |
| ANISOU | 2615 | N | ARG | B | 170 | 1325 | 2784 | 1297 | −106 | −117 | 15 | N |
| ATOM | 2616 | CA | ARG | B | 170 | −10.866 | −13.700 | −7.978 | 1.00 | 14.07 | | C |
| ANISOU | 2616 | CA | ARG | B | 170 | 1113 | 3009 | 1225 | −212 | −111 | −41 | C |
| ATOM | 2617 | C | ARG | B | 170 | −9.782 | −14.756 | −7.851 | 1.00 | 12.59 | | C |
| ANISOU | 2617 | C | ARG | B | 170 | 1175 | 2580 | 1029 | −408 | 171 | 101 | C |
| ATOM | 2618 | O | ARG | B | 170 | −10.095 | −15.911 | −7.533 | 1.00 | 16.82 | | O |
| ANISOU | 2618 | O | ARG | B | 170 | 1594 | 2830 | 1967 | −987 | −994 | 587 | O |
| ATOM | 2619 | CB | ARG | B | 170 | −11.280 | −13.526 | −9.430 | 1.00 | 14.73 | | C |
| ANISOU | 2619 | CB | ARG | B | 170 | 1200 | 3240 | 1157 | −253 | −31 | −33 | C |
| ATOM | 2620 | CG | ARG | B | 170 | −11.700 | −14.791 | −10.155 | 1.00 | 17.83 | | C |
| ANISOU | 2620 | CG | ARG | B | 170 | 1687 | 3643 | 1444 | −718 | −108 | −262 | C |
| ATOM | 2621 | CD | ARG | B | 170 | −12.860 | −15.534 | −9.499 | 1.00 | 17.01 | | C |
| ANISOU | 2621 | CD | ARG | B | 170 | 1500 | 3365 | 1598 | −458 | −544 | 457 | C |
| ATOM | 2622 | NE | ARG | B | 170 | −14.112 | −14.856 | −9.575 | 1.00 | 18.42 | | N |
| ANISOU | 2622 | NE | ARG | B | 170 | 1569 | 3811 | 1620 | −186 | −279 | 404 | N |
| ATOM | 2623 | CZ | ARG | B | 170 | −15.257 | −15.081 | −8.961 | 1.00 | 18.74 | | C |
| ANISOU | 2623 | CZ | ARG | B | 170 | 2127 | 3628 | 1366 | 7 | 329 | −268 | C |
| ATOM | 2624 | NH1 | ARG | B | 170 | −15.347 | −16.044 | −8.112 | 1.00 | 19.47 | | N |
| ANISOU | 2624 | NH1 | ARG | B | 170 | 2388 | 3042 | 1967 | −555 | 403 | −385 | N |
| ATOM | 2625 | NH2 | ARG | B | 170 | −16.314 | −14.313 | −9.220 | 1.00 | 20.43 | | N |
| ANISOU | 2625 | NH2 | ARG | B | 170 | 1867 | 4024 | 1870 | 14 | 436 | −294 | N |
| ATOM | 2626 | N | ARG | B | 171 | −8.517 | −14.374 | −8.094 | 1.00 | 12.58 | | N |
| ANISOU | 2626 | N | ARG | B | 171 | 1073 | 2344 | 1362 | −317 | −87 | 157 | N |
| ATOM | 2627 | CA | ARG | B | 171 | −7.453 | −15.364 | −8.027 | 1.00 | 13.10 | | C |
| ANISOU | 2627 | CA | ARG | B | 171 | 1238 | 2079 | 1660 | −280 | −107 | −76 | C |
| ATOM | 2628 | C | ARG | B | 171 | −7.255 | −15.909 | −6.617 | 1.00 | 12.83 | | C |
| ANISOU | 2628 | C | ARG | B | 171 | 1056 | 1908 | 1911 | −405 | −213 | 112 | C |
| ATOM | 2629 | O | ARG | B | 171 | −6.896 | −17.067 | −6.424 | 1.00 | 15.63 | | O |
| ANISOU | 2629 | O | ARG | B | 171 | 1594 | 2054 | 2290 | −126 | −557 | 60 | O |
| ATOM | 2630 | CB | ARG | B | 171 | −6.149 | −14.768 | −8.582 | 1.00 | 12.07 | | C |
| ANISOU | 2630 | CB | ARG | B | 171 | 1178 | 1760 | 1649 | −119 | −83 | −87 | C |
| ATOM | 2631 | CG | ARG | B | 171 | −6.130 | −14.621 | −10.124 | 1.00 | 14.98 | | C |
| ANISOU | 2631 | CG | ARG | B | 171 | 1822 | 2068 | 1803 | 450 | −74 | 748 | C |
| ATOM | 2632 | CD | ARG | B | 171 | −5.829 | −15.953 | −10.775 | 1.00 | 26.95 | | C |
| ANISOU | 2632 | CD | ARG | B | 171 | 4330 | 3121 | 2788 | −2427 | 1475 | −1585 | C |
| ATOM | 2633 | NE | ARG | B | 171 | −6.071 | −16.393 | −12.086 | 1.00 | 49.36 | | N |
| ANISOU | 2633 | NE | ARG | B | 171 | 9179 | 5985 | 3589 | −2814 | −626 | −2054 | N |
| ATOM | 2634 | CZ | ARG | B | 171 | −6.765 | −17.391 | −12.555 | 1.00 | 51.02 | | C |
| ANISOU | 2634 | CZ | ARG | B | 171 | 10378 | 5689 | 3319 | −2866 | −961 | −2055 | C |
| ATOM | 2635 | NH1 | ARG | B | 171 | −7.445 | −18.245 | −11.789 | 1.00 | 56.49 | | N |
| ANISOU | 2635 | NH1 | ARG | B | 171 | 8282 | 6582 | 6600 | −2675 | 2727 | −3272 | N |
| ATOM | 2636 | NH2 | ARG | B | 171 | −6.782 | −17.533 | −13.876 | 1.00 | 69.77 | | N |
| ANISOU | 2636 | NH2 | ARG | B | 171 | 14995 | 8073 | 3442 | −2277 | −2026 | −2627 | N |
| ATOM | 2637 | N | VAL | B | 172 | −7.489 | −15.121 | −5.586 | 1.00 | 13.29 | | N |
| ANISOU | 2637 | N | VAL | B | 172 | 1327 | 2169 | 1555 | −151 | −454 | 179 | N |
| ATOM | 2638 | CA | VAL | B | 172 | −7.412 | −15.571 | −4.211 | 1.00 | 15.83 | | C |
| ANISOU | 2638 | CA | VAL | B | 172 | 1365 | 2903 | 1748 | −424 | −484 | 590 | C |
| ATOM | 2639 | C | VAL | B | 172 | −8.485 | −16.622 | −3.961 | 1.00 | 20.87 | | C |
| ANISOU | 2639 | C | VAL | B | 172 | 2099 | 3345 | 2485 | −1102 | −1068 | 1130 | C |
| ATOM | 2640 | O | VAL | B | 172 | −8.246 | −17.689 | −3.401 | 1.00 | 24.03 | | O |
| ANISOU | 2640 | O | VAL | B | 172 | 2521 | 3597 | 3012 | −1116 | −1081 | 1515 | O |
| ATOM | 2641 | CB | VAL | B | 172 | −7.550 | −14.386 | −3.236 | 1.00 | 14.92 | | C |
| ANISOU | 2641 | CB | VAL | B | 172 | 1007 | 3370 | 1292 | −543 | −64 | 464 | C |
| ATOM | 2642 | CG1 | VAL | B | 172 | −7.584 | −14.884 | −1.783 | 1.00 | 20.55 | | C |
| ANISOU | 2642 | CG1 | VAL | B | 172 | 1746 | 4652 | 1411 | −1432 | −435 | 904 | C |
| ATOM | 2643 | CG2 | VAL | B | 172 | −6.424 | −13.383 | −3.408 | 1.00 | 14.48 | | C |
| ANISOU | 2643 | CG2 | VAL | B | 172 | 1103 | 3036 | 1364 | −457 | −280 | 572 | C |
| ATOM | 2644 | N | LEU | B | 173 | −9.700 | −16.330 | −4.428 | 1.00 | 17.52 | | N |
| ANISOU | 2644 | N | LEU | B | 173 | 1747 | 2835 | 2074 | −997 | −502 | 549 | N |
| ATOM | 2645 | CA | LEU | B | 173 | −10.727 | −17.383 | −4.342 | 1.00 | 19.94 | | C |
| ANISOU | 2645 | CA | LEU | B | 173 | 1823 | 2839 | 2913 | −994 | −343 | 568 | C |
| ATOM | 2646 | C | LEU | B | 173 | −10.404 | −18.647 | −5.105 | 1.00 | 21.36 | | C |
| ANISOU | 2646 | C | LEU | B | 173 | 1906 | 2613 | 3595 | −813 | −848 | 522 | C |
| ATOM | 2647 | O | LEU | B | 173 | −10.690 | −19.764 | −4.643 | 1.00 | 33.16 | | O |
| ANISOU | 2647 | O | LEU | B | 173 | 4538 | 2696 | 5366 | −1667 | −1410 | 835 | O |
| ATOM | 2648 | CB | LEU | B | 173 | −12.027 | −16.757 | −4.850 | 1.00 | 20.47 | | C |
| ANISOU | 2648 | CB | LEU | B | 173 | 1435 | 3409 | 2932 | −744 | 48 | 14 | C |
| ATOM | 2649 | CG | LEU | B | 173 | −12.567 | −15.616 | −3.987 | 1.00 | 22.93 | | C |
| ANISOU | 2649 | CG | LEU | B | 173 | 2123 | 3472 | 3119 | −708 | −229 | −329 | C |
| ATOM | 2650 | CD1 | LEU | B | 173 | −13.865 | −15.125 | −4.631 | 1.00 | 33.67 | | C |
| ANISOU | 2650 | CD1 | LEU | B | 173 | 3330 | 5380 | 4083 | 1216 | −653 | −799 | C |
| ATOM | 2651 | CD2 | LEU | B | 173 | −12.769 | −16.018 | −2.542 | 1.00 | 28.74 | | C |
| ANISOU | 2651 | CD2 | LEU | B | 173 | 3348 | 4882 | 2690 | −1423 | −442 | −867 | C |
| ATOM | 2652 | N | ALA | B | 174 | −9.799 | −18.478 | −6.282 | 1.00 | 25.57 | | N |
| ANISOU | 2652 | N | ALA | B | 174 | 2049 | 3868 | 3797 | −872 | −324 | −473 | N |
| ATOM | 2653 | CA | ALA | B | 174 | −9.561 | −19.634 | −7.132 | 1.00 | 28.19 | | C |
| ANISOU | 2653 | CA | ALA | B | 174 | 2748 | 3055 | 4907 | −1018 | 219 | −271 | C |
| ATOM | 2654 | C | ALA | B | 174 | −8.458 | −20.513 | −6.566 | 1.00 | 35.01 | | C |
| ANISOU | 2654 | C | ALA | B | 174 | 4141 | 2728 | 6435 | −850 | −744 | 104 | C |
| ATOM | 2655 | O | ALA | B | 174 | −8.445 | −21.736 | −6.754 | 1.00 | 51.65 | | O |
| ANISOU | 2655 | O | ALA | B | 174 | 7741 | 2548 | 9338 | −719 | −1943 | 529 | O |
| ATOM | 2656 | CB | ALA | B | 174 | −9.231 | −19.112 | −8.520 | 1.00 | 34.35 | | C |
| ANISOU | 2656 | CB | ALA | B | 174 | 4710 | 3967 | 4374 | 510 | 639 | −599 | C |
| ATOM | 2657 | N | THR | B | 175 | −7.506 | −19.935 | −5.852 | 1.00 | 40.57 | | N |
| ANISOU | 2657 | N | THR | B | 175 | 4377 | 4208 | 6828 | −913 | −1597 | 439 | N |
| ATOM | 2658 | CA | THR | B | 175 | −6.272 | −20.574 | −5.426 | 1.00 | 39.34 | | C |
| ANISOU | 2658 | CA | THR | B | 175 | 5114 | 3961 | 5871 | −735 | −1459 | 1772 | C |
| ATOM | 2659 | C | THR | B | 175 | −6.340 | −21.181 | −4.027 | 1.00 | 40.87 | | C |
| ANISOU | 2659 | C | THR | B | 175 | 5217 | 4517 | 5797 | −2838 | −1130 | 1682 | C |
| ATOM | 2660 | O | THR | B | 175 | −7.470 | −21.284 | −3.513 | 1.00 | 51.29 | | O |
| ANISOU | 2660 | O | THR | B | 175 | 6182 | 7538 | 5768 | −3900 | 54 | −1633 | O |
| ATOM | 2661 | CB | THR | B | 175 | −5.121 | −19.543 | −5.473 | 1.00 | 35.36 | | C |
| ANISOU | 2661 | CB | THR | B | 175 | 3476 | 4794 | 5164 | −106 | 103 | 2415 | C |
| ATOM | 2662 | OG1 | THR | B | 175 | −4.863 | −19.266 | −6.852 | 1.00 | 40.79 | | O |
| ANISOU | 2662 | OG1 | THR | B | 175 | 5794 | 5084 | 4621 | 488 | 1093 | 364 | O |
| ATOM | 2663 | CG2 | THR | B | 175 | −3.854 | −20.138 | −4.900 | 1.00 | 47.92 | | C |
| ANISOU | 2663 | CG2 | THR | B | 175 | 4032 | 6555 | 7622 | 1105 | −809 | 1148 | C |
| TER | 2664 | | THR | B | 175 | | | | | | | |
| HETATM | 2665 | PB | GDP | | 1001 | −3.289 | 15.890 | 8.746 | 1.00 | 9.89 | | P |
| ANISOU | 2665 | PB | GDP | | 1001 | 880 | 1717 | 1159 | −81 | −190 | −279 | P |
| HETATM | 2666 | O1B | GDP | | 1001 | −4.674 | 15.715 | 8.204 | 1.00 | 11.50 | | O |
| ANISOU | 2666 | O1B | GDP | | 1001 | 903 | 2003 | 1464 | −41 | −349 | −244 | O |
| HETATM | 2667 | O2B | GDP | | 1001 | −3.292 | 16.060 | 10.235 | 1.00 | 8.68 | | O |
| ANISOU | 2667 | O2B | GDP | | 1001 | 725 | 1524 | 1047 | −60 | −20 | −131 | O |
| HETATM | 2668 | O3B | GDP | | 1001 | −2.294 | 14.892 | 8.241 | 1.00 | 10.16 | | O |
| ANISOU | 2668 | O3B | GDP | | 1001 | 851 | 1721 | 1289 | −156 | −151 | −608 | O |
| HETATM | 2669 | O3A | GDP | | 1001 | −2.813 | 17.371 | 8.199 | 1.00 | 10.11 | | O |
| ANISOU | 2669 | O3A | GDP | | 1001 | 1033 | 1793 | 1014 | −118 | −251 | −64 | O |
| HETATM | 2670 | PA | GDP | | 1001 | −1.849 | 17.715 | 7.004 | 1.00 | 10.62 | | P |
| ANISOU | 2670 | PA | GDP | | 1001 | 1016 | 1818 | 1201 | 80 | −156 | −161 | P |
| HETATM | 2671 | O1A | GDP | | 1001 | −0.424 | 17.465 | 7.333 | 1.00 | 11.37 | | O |
| ANISOU | 2671 | O1A | GDP | | 1001 | 1015 | 1857 | 1447 | 65 | −78 | −147 | O |
| HETATM | 2672 | O2A | GDP | | 1001 | −2.378 | 17.171 | 5.736 | 1.00 | 13.15 | | O |
| ANISOU | 2672 | O2A | GDP | | 1001 | 1571 | 2339 | 1087 | 18 | −157 | −281 | O |
| HETATM | 2673 | O5* | GDP | | 1001 | −1.956 | 19.287 | 6.965 | 1.00 | 11.08 | | O |
| ANISOU | 2673 | O5* | GDP | | 1001 | 877 | 1811 | 1521 | −71 | −257 | 108 | O |
| HETATM | 2674 | C5* | GDP | | 1001 | −3.319 | 19.921 | 6.875 | 1.00 | 10.60 | | C |
| ANISOU | 2674 | C5* | GDP | | 1001 | 861 | 1727 | 1440 | −48 | −79 | 172 | C |
| HETATM | 2675 | C4* | GDP | | 1001 | −3.066 | 21.227 | 6.201 | 1.00 | 10.65 | | C |
| ANISOU | 2675 | C4* | GDP | | 1001 | 1332 | 1622 | 1094 | 0 | −123 | −41 | C |
| HETATM | 2676 | O4* | GDP | | 1001 | −2.374 | 22.165 | 7.153 | 1.00 | 10.94 | | O |
| ANISOU | 2676 | O4* | GDP | | 1001 | 1128 | 1798 | 1231 | −238 | −255 | 171 | O |
| HETATM | 2677 | C3* | GDP | | 1001 | −2.186 | 21.230 | 4.912 | 1.00 | 11.95 | | C |
| ANISOU | 2677 | C3* | GDP | | 1001 | 1147 | 2361 | 1032 | −31 | −226 | 357 | C |
| HETATM | 2678 | O3* | GDP | | 1001 | −2.726 | 22.363 | 4.099 | 1.00 | 14.46 | | O |
| ANISOU | 2678 | O3* | GDP | | 1001 | 1785 | 2302 | 1405 | −116 | −539 | 459 | O |
| HETATM | 2679 | C2* | GDP | | 1001 | −0.802 | 21.597 | 5.497 | 1.00 | 11.89 | | C |
| ANISOU | 2679 | C2* | GDP | | 1001 | 1353 | 2072 | 1094 | −235 | −221 | 181 | C |
| HETATM | 2680 | O2* | GDP | | 1001 | 0.034 | 22.229 | 4.511 | 1.00 | 12.49 | | O |
| ANISOU | 2680 | O2* | GDP | | 1001 | 1261 | 2087 | 1396 | −136 | −61 | 156 | O |
| HETATM | 2681 | C1* | GDP | | 1001 | −1.167 | 22.553 | 6.574 | 1.00 | 11.76 | | C |
| ANISOU | 2681 | C1* | GDP | | 1001 | 1053 | 2162 | 1253 | −32 | −206 | 191 | C |
| HETATM | 2682 | N9 | GDP | | 1001 | −0.093 | 22.623 | 7.640 | 1.00 | 9.53 | | N |
| ANISOU | 2682 | N9 | GDP | | 1001 | 1036 | 1456 | 1130 | 0 | −77 | 105 | N |
| HETATM | 2683 | C8 | GDP | | 1001 | 0.438 | 21.414 | 8.222 | 1.00 | 9.91 | | C |
| ANISOU | 2683 | C8 | GDP | | 1001 | 1119 | 1495 | 1152 | 91 | −148 | 12 | C |
| HETATM | 2684 | N7 | GDP | | 1001 | 1.305 | 21.870 | 9.202 | 1.00 | 10.41 | | N |
| ANISOU | 2684 | N7 | GDP | | 1001 | 977 | 1428 | 1551 | 80 | −198 | 26 | N |
| HETATM | 2685 | C5 | GDP | | 1001 | 1.268 | 23.214 | 9.190 | 1.00 | 9.44 | | C |
| ANISOU | 2685 | C5 | GDP | | 1001 | 1083 | 1381 | 1124 | 5 | 11 | 108 | C |
| HETATM | 2686 | C6 | GDP | | 1001 | 1.937 | 24.212 | 9.878 | 1.00 | 10.11 | | C |
| ANISOU | 2686 | C6 | GDP | | 1001 | 1237 | 1477 | 1126 | 172 | −162 | −74 | C |
| HETATM | 2687 | O6 | GDP | | 1001 | 2.835 | 23.905 | 10.813 | 1.00 | 11.17 | | O |
| ANISOU | 2687 | O6 | GDP | | 1001 | 1173 | 1872 | 1198 | 227 | −153 | 77 | O |
| HETATM | 2688 | N1 | GDP | | 1001 | 1.656 | 25.502 | 9.629 | 1.00 | 9.42 | | N |
| ANISOU | 2688 | N1 | GDP | | 1001 | 764 | 1443 | 1371 | −48 | 6 | 195 | N |
| HETATM | 2689 | C2 | GDP | | 1001 | 0.706 | 25.789 | 8.728 | 1.00 | 9.75 | | C |
| ANISOU | 2689 | C2 | GDP | | 1001 | 871 | 1442 | 1391 | −69 | −91 | 156 | C |
| HETATM | 2690 | N2 | GDP | | 1001 | 0.572 | 27.111 | 8.576 | 1.00 | 11.20 | | N |
| ANISOU | 2690 | N2 | GDP | | 1001 | 1349 | 1449 | 1458 | 72 | −311 | 100 | N |
| HETATM | 2691 | N3 | GDP | | 1001 | 0.049 | 24.971 | 7.969 | 1.00 | 9.74 | | N |
| ANISOU | 2691 | N3 | GDP | | 1001 | 1254 | 1404 | 1043 | −90 | −71 | 196 | N |
| HETATM | 2692 | C4 | GDP | | 1001 | 0.339 | 23.763 | 8.209 | 1.00 | 9.27 | | C |
| ANISOU | 2692 | C4 | GDP | | 1001 | 863 | 1438 | 1222 | −115 | 45 | 248 | C |
| HETATM | 2693 | PB | GDP | | 1002 | 5.686 | 4.378 | −8.009 | 1.00 | 11.83 | | P |
| ANISOU | 2693 | PB | GDP | | 1002 | 1183 | 1631 | 1682 | −86 | −200 | −297 | P |
| HETATM | 2694 | O1B | GDP | | 1002 | 7.048 | 4.795 | −7.654 | 1.00 | 12.96 | | O |
| ANISOU | 2694 | O1B | GDP | | 1002 | 1296 | 1634 | 1994 | −204 | −304 | −286 | O |
| HETATM | 2695 | O2B | GDP | | 1002 | 5.720 | 3.030 | −8.697 | 1.00 | 10.58 | | O |
| ANISOU | 2695 | O2B | GDP | | 1002 | 1065 | 1551 | 1404 | −99 | −228 | −156 | O |
| HETATM | 2696 | O3B | GDP | | 1002 | 4.735 | 4.522 | −6.924 | 1.00 | 12.30 | | O |
| ANISOU | 2696 | O3B | GDP | | 1002 | 1333 | 1704 | 1635 | 72 | −158 | −223 | O |
| HETATM | 2697 | O3A | GDP | | 1002 | 5.158 | 5.405 | −9.224 | 1.00 | 11.73 | | O |
| ANISOU | 2697 | O3A | GDP | | 1002 | 1182 | 1479 | 1796 | −220 | −105 | −143 | O |
| HETATM | 2698 | PA | GDP | | 1002 | 4.225 | 6.660 | −9.161 | 1.00 | 13.27 | | P |
| ANISOU | 2698 | PA | GDP | | 1002 | 1476 | 1468 | 2099 | −133 | −131 | −157 | P |
| HETATM | 2699 | O1A | GDP | | 1002 | 2.851 | 6.229 | −9.050 | 1.00 | 13.18 | | O |
| ANISOU | 2699 | O1A | GDP | | 1002 | 1388 | 1729 | 1891 | 195 | 42 | −99 | O |
| HETATM | 2700 | O2A | GDP | | 1002 | 4.734 | 7.696 | −8.115 | 1.00 | 17.05 | | O |
| ANISOU | 2700 | O2A | GDP | | 1002 | 2429 | 1589 | 2459 | −50 | −423 | −409 | O |
| HETATM | 2701 | O5* | GDP | | 1002 | 4.331 | 7.293 | −10.653 | 1.00 | 14.51 | | O |
| ANISOU | 2701 | O5* | GDP | | 1002 | 1289 | 1960 | 2264 | −274 | −425 | 237 | O |
| HETATM | 2702 | C5* | GDP | | 1002 | 5.740 | 7.638 | −11.247 | 1.00 | 14.44 | | C |
| ANISOU | 2702 | C5* | GDP | | 1002 | 1473 | 1836 | 2178 | −415 | −392 | 290 | C |
| HETATM | 2703 | C4* | GDP | | 1002 | 5.499 | 8.684 | −12.287 | 1.00 | 13.74 | | C |
| ANISOU | 2703 | C4* | GDP | | 1002 | 1768 | 1753 | 1698 | −45 | −214 | −13 | C |
| HETATM | 2704 | O4* | GDP | | 1002 | 4.766 | 8.020 | −13.309 | 1.00 | 14.01 | | O |
| ANISOU | 2704 | O4* | GDP | | 1002 | 1760 | 1342 | 2220 | −117 | −529 | 89 | O |
| HETATM | 2705 | C3* | GDP | | 1002 | 4.605 | 9.783 | −11.775 | 1.00 | 15.76 | | C |
| ANISOU | 2705 | C3* | GDP | | 1002 | 1698 | 1668 | 2624 | −180 | −91 | −232 | C |
| HETATM | 2706 | O3* | GDP | | 1002 | 5.036 | 10.982 | −12.608 | 1.00 | 21.20 | | O |
| ANISOU | 2706 | O3* | GDP | | 1002 | 3163 | 1543 | 3348 | −632 | −1137 | 149 | O |
| HETATM | 2707 | C2* | GDP | | 1002 | 3.123 | 9.402 | −12.253 | 1.00 | 17.36 | | C |
| ANISOU | 2707 | C2* | GDP | | 1002 | 1893 | 1578 | 3124 | 156 | −718 | −355 | C |
| HETATM | 2708 | O2* | GDP | | 1002 | 2.354 | 10.500 | −12.631 | 1.00 | 21.51 | | O |
| ANISOU | 2708 | O2* | GDP | | 1002 | 2624 | 1691 | 3856 | 366 | −1054 | −269 | O |
| HETATM | 2709 | C1* | GDP | | 1002 | 3.497 | 8.676 | −13.568 | 1.00 | 15.02 | | C |
| ANISOU | 2709 | C1* | GDP | | 1002 | 1746 | 1424 | 2535 | −219 | −472 | 293 | C |
| HETATM | 2710 | N9 | GDP | | 1002 | 2.496 | 7.751 | −13.965 | 1.00 | 13.15 | | N |
| ANISOU | 2710 | N9 | GDP | | 1002 | 1600 | 1311 | 2088 | −116 | −278 | 276 | N |
| HETATM | 2711 | C8 | GDP | | 1002 | 2.016 | 6.748 | −13.032 | 1.00 | 13.06 | | C |
| ANISOU | 2711 | C8 | GDP | | 1002 | 1818 | 1138 | 2006 | −73 | −466 | 286 | C |
| HETATM | 2712 | N7 | GDP | | 1002 | 1.097 | 5.989 | −13.818 | 1.00 | 13.10 | | N |
| ANISOU | 2712 | N7 | GDP | | 1002 | 1680 | 1695 | 1604 | −307 | −49 | 129 | N |
| HETATM | 2713 | C5 | GDP | | 1002 | 1.113 | 6.483 | −15.161 | 1.00 | 12.52 | | C |
| ANISOU | 2713 | C5 | GDP | | 1002 | 1343 | 1691 | 1722 | −169 | −95 | 369 | C |
| HETATM | 2714 | C6 | GDP | | 1002 | 0.438 | 6.094 | −16.307 | 1.00 | 15.25 | | C |
| ANISOU | 2714 | C6 | GDP | | 1002 | 1870 | 2119 | 1805 | −415 | −386 | 405 | C |
| HETATM | 2715 | O6 | GDP | | 1002 | −0.444 | 5.264 | −16.298 | 1.00 | 13.57 | | O |
| ANISOU | 2715 | O6 | GDP | | 1002 | 1662 | 1666 | 1830 | −55 | −278 | 198 | O |
| HETATM | 2716 | N1 | GDP | | 1002 | 0.657 | 6.827 | −17.360 | 1.00 | 12.99 | | N |
| ANISOU | 2716 | N1 | GDP | | 1002 | 1715 | 1500 | 1720 | 132 | −160 | 187 | N |
| HETATM | 2717 | C2 | GDP | | 1002 | 1.547 | 7.892 | −17.370 | 1.00 | 14.09 | | C |
| ANISOU | 2717 | C2 | GDP | | 1002 | 1691 | 1668 | 1993 | 108 | −130 | 409 | C |
| HETATM | 2718 | N2 | GDP | | 1002 | 1.793 | 8.518 | −18.589 | 1.00 | 16.69 | | N |
| ANISOU | 2718 | N2 | GDP | | 1002 | 2105 | 1958 | 2277 | 75 | −210 | 785 | N |
| HETATM | 2719 | N3 | GDP | | 1002 | 2.324 | 8.381 | −16.313 | 1.00 | 14.52 | | N |
| ANISOU | 2719 | N3 | GDP | | 1002 | 1706 | 1576 | 2235 | −249 | −209 | 573 | N |
| HETATM | 2720 | C4 | GDP | | 1002 | 2.039 | 7.641 | −15.243 | 1.00 | 13.94 | | C |
| ANISOU | 2720 | C4 | GDP | | 1002 | 1413 | 1823 | 2060 | −273 | −266 | 510 | C |
| HETATM | 2721 | O | HOH | | 1 | −3.169 | 9.524 | 12.530 | 1.00 | 13.32 | | O |
| ANISOU | 2721 | O | HOH | | 1 | 1438 | 1771 | 1850 | 166 | −578 | −677 | O |
| HETATM | 2722 | O | HOH | | 2 | −6.776 | 16.878 | 17.504 | 1.00 | 9.79 | | O |
| ANISOU | 2722 | O | HOH | | 2 | 593 | 1836 | 1290 | −42 | −318 | −431 | O |
| HETATM | 2723 | O | HOH | | 3 | 11.873 | −11.081 | −15.279 | 1.00 | 13.15 | | O |
| ANISOU | 2723 | O | HOH | | 3 | 1325 | 2425 | 1248 | 169 | −286 | −191 | O |
| HETATM | 2724 | O | HOH | | 4 | 5.513 | −1.463 | −3.423 | 1.00 | 12.58 | | O |
| ANISOU | 2724 | O | HOH | | 4 | 1581 | 2012 | 1185 | −503 | −333 | −346 | O |
| HETATM | 2725 | O | HOH | | 5 | 9.167 | −3.525 | −11.945 | 1.00 | 11.07 | | O |
| ANISOU | 2725 | O | HOH | | 5 | 1056 | 1749 | 1401 | −389 | −307 | −236 | O |
| HETATM | 2726 | O | HOH | | 6 | 12.660 | −16.673 | −3.584 | 1.00 | 14.18 | | O |
| ANISOU | 2726 | O | HOH | | 6 | 1445 | 1801 | 2140 | 138 | −72 | −507 | O |
| HETATM | 2727 | O | HOH | | 7 | −2.851 | 25.345 | 7.694 | 1.00 | 12.57 | | O |
| ANISOU | 2727 | O | HOH | | 7 | 1415 | 1801 | 1559 | 81 | −351 | 445 | O |
| HETATM | 2728 | O | HOH | | 8 | 8.418 | −3.496 | −1.619 | 1.00 | 14.27 | | O |
| ANISOU | 2728 | O | HOH | | 8 | 1439 | 1708 | 2276 | −39 | −204 | −145 | O |
| HETATM | 2729 | O | HOH | | 9 | −12.082 | 15.100 | 23.983 | 1.00 | 13.79 | | O |
| ANISOU | 2729 | O | HOH | | 9 | 1299 | 1957 | 1986 | −112 | 59 | −513 | O |
| HETATM | 2730 | O | HOH | | 10 | 10.195 | −10.138 | −17.309 | 1.00 | 14.54 | | O |
| ANISOU | 2730 | O | HOH | | 10 | 1593 | 2030 | 1903 | 5 | −728 | −413 | O |
| HETATM | 2731 | O | HOH | | 11 | 14.470 | −10.223 | −12.620 | 1.00 | 12.73 | | O |
| ANISOU | 2731 | O | HOH | | 11 | 1273 | 2272 | 1292 | 93 | −146 | −409 | O |
| HETATM | 2732 | O | HOH | | 12 | −7.743 | 19.470 | 25.563 | 1.00 | 14.25 | | O |
| ANISOU | 2732 | O | HOH | | 12 | 1714 | 2175 | 1525 | −636 | −199 | −20 | O |
| HETATM | 2733 | O | HOH | | 13 | 5.997 | 18.625 | 5.755 | 1.00 | 15.64 | | O |
| ANISOU | 2733 | O | HOH | | 13 | 1203 | 2849 | 1890 | −8 | −262 | 632 | O |
| HETATM | 2734 | O | HOH | | 14 | 9.999 | −0.504 | −5.297 | 1.00 | 16.62 | | O |
| ANISOU | 2734 | O | HOH | | 14 | 2862 | 1630 | 1822 | −328 | −346 | −439 | O |
| HETATM | 2735 | O | HOH | | 15 | −10.195 | 4.562 | 26.774 | 1.00 | 14.21 | | O |
| ANISOU | 2735 | O | HOH | | 15 | 1423 | 2195 | 1780 | −112 | 12 | −12 | O |
| HETATM | 2736 | O | HOH | | 16 | 9.131 | −2.781 | 10.536 | 1.00 | 14.96 | | O |
| ANISOU | 2736 | O | HOH | | 16 | 1892 | 1945 | 1847 | −216 | 49 | −486 | O |
| HETATM | 2737 | O | HOH | | 17 | 7.001 | 24.540 | 22.988 | 1.00 | 14.12 | | O |
| ANISOU | 2737 | O | HOH | | 17 | 1821 | 1872 | 1670 | −484 | −544 | −348 | O |
| HETATM | 2738 | O | HOH | | 18 | −14.799 | 1.539 | −0.800 | 1.00 | 16.80 | | O |
| ANISOU | 2738 | O | HOH | | 18 | 1596 | 1990 | 2795 | 132 | −565 | −224 | O |
| HETATM | 2739 | O | HOH | | 19 | −9.451 | 17.328 | 25.621 | 1.00 | 14.23 | | O |
| ANISOU | 2739 | O | HOH | | 19 | 1243 | 2054 | 2109 | −73 | 154 | −193 | O |
| HETATM | 2740 | O | HOH | | 20 | 3.570 | −2.395 | 13.731 | 1.00 | 16.91 | | O |
| ANISOU | 2740 | O | HOH | | 20 | 2187 | 1723 | 2516 | 295 | 80 | −635 | O |
| HETATM | 2741 | O | HOH | | 21 | −11.248 | −1.903 | −15.496 | 1.00 | 16.21 | | O |
| ANISOU | 2741 | O | HOH | | 21 | 1958 | 2590 | 1612 | 358 | −365 | −149 | O |
| HETATM | 2742 | O | HOH | | 22 | −0.989 | −15.041 | −24.084 | 1.00 | 15.28 | | O |
| ANISOU | 2742 | O | HOH | | 22 | 981 | 2048 | 2778 | 72 | −255 | −339 | O |
| HETATM | 2743 | O | HOH | | 23 | 2.679 | 29.818 | 29.389 | 1.00 | 18.26 | | O |
| ANISOU | 2743 | O | HOH | | 23 | 1396 | 3402 | 2140 | −178 | −764 | −618 | O |
| HETATM | 2744 | O | HOH | | 24 | 12.748 | 20.565 | 13.783 | 1.00 | 15.79 | | O |
| ANISOU | 2744 | O | HOH | | 24 | 1513 | 2302 | 2186 | 150 | 315 | 492 | O |
| HETATM | 2745 | O | HOH | | 25 | −7.491 | 11.569 | 12.284 | 1.00 | 19.44 | | O |
| ANISOU | 2745 | O | HOH | | 25 | 2456 | 3085 | 1844 | 284 | −243 | −192 | O |
| HETATM | 2746 | O | HOH | | 26 | −0.250 | −9.883 | −28.571 | 1.00 | 15.63 | | O |
| ANISOU | 2746 | O | HOH | | 26 | 1504 | 2210 | 2224 | −222 | −487 | −140 | O |
| HETATM | 2747 | O | HOH | | 27 | −6.730 | −3.922 | 8.767 | 1.00 | 17.26 | | O |
| ANISOU | 2747 | O | HOH | | 27 | 2028 | 2851 | 1677 | 707 | −71 | −773 | O |
| HETATM | 2748 | O | HOH | | 28 | 2.912 | 18.856 | 31.381 | 1.00 | 15.55 | | O |
| ANISOU | 2748 | O | HOH | | 28 | 1793 | 1938 | 2178 | −167 | 98 | −247 | O |
| HETATM | 2749 | O | HOH | | 29 | 1.715 | 20.020 | 4.016 | 1.00 | 24.29 | | O |
| ANISOU | 2749 | O | HOH | | 29 | 1569 | 4846 | 2813 | −86 | −11 | 503 | O |
| HETATM | 2750 | O | HOH | | 30 | 17.175 | 8.189 | 8.809 | 1.00 | 16.85 | | O |
| ANISOU | 2750 | O | HOH | | 30 | 1804 | 2445 | 2152 | −480 | 68 | 230 | O |
| HETATM | 2751 | O | HOH | | 31 | 3.369 | 23.809 | 32.656 | 1.00 | 15.85 | | O |
| ANISOU | 2751 | O | HOH | | 31 | 1237 | 2759 | 2027 | 9 | −269 | −156 | O |
| HETATM | 2752 | O | HOH | | 32 | −4.482 | −5.965 | −21.198 | 1.00 | 15.12 | | O |
| ANISOU | 2752 | O | HOH | | 32 | 1706 | 2288 | 1753 | −553 | −525 | −17 | O |
| HETATM | 2753 | O | HOH | | 33 | 3.113 | −3.710 | −28.167 | 1.00 | 19.74 | | O |
| ANISOU | 2753 | O | HOH | | 33 | 2509 | 3358 | 1633 | −117 | −447 | 766 | O |
| HETATM | 2754 | O | HOH | | 34 | 4.153 | −21.084 | −16.974 | 1.00 | 17.95 | | O |
| ANISOU | 2754 | O | HOH | | 34 | 2219 | 1791 | 2811 | −256 | −585 | −97 | O |
| HETATM | 2755 | O | HOH | | 35 | 13.615 | 20.727 | 17.133 | 1.00 | 14.25 | | O |
| ANISOU | 2755 | O | HOH | | 35 | 1742 | 1413 | 2259 | −78 | −50 | 17 | O |
| HETATM | 2756 | O | HOH | | 36 | −0.604 | 31.826 | 23.438 | 1.00 | 18.96 | | O |
| ANISOU | 2756 | O | HOH | | 36 | 2066 | 1767 | 3370 | −225 | −365 | 273 | O |
| HETATM | 2757 | O | HOH | | 37 | 11.319 | 0.373 | 2.590 | 1.00 | 20.39 | | O |
| ANISOU | 2757 | O | HOH | | 37 | 1966 | 2302 | 3479 | 8 | 210 | −350 | O |
| HETATM | 2758 | O | HOH | | 38 | −14.910 | 0.926 | 9.910 | 1.00 | 17.57 | | O |
| ANISOU | 2758 | O | HOH | | 38 | 1300 | 2502 | 2872 | −372 | −403 | 42 | O |
| HETATM | 2759 | O | HOH | | 39 | −1.833 | 15.121 | 35.670 | 1.00 | 16.43 | | O |
| ANISOU | 2759 | O | HOH | | 39 | 2253 | 2430 | 1560 | −314 | −99 | −213 | O |
| HETATM | 2760 | O | HOH | | 40 | −21.063 | −9.763 | −6.818 | 1.00 | 15.64 | | O |
| ANISOU | 2760 | O | HOH | | 40 | 1351 | 2955 | 1639 | −112 | −402 | −234 | O |
| HETATM | 2761 | O | HOH | | 41 | −5.984 | 7.061 | 13.788 | 1.00 | 16.86 | | O |
| ANISOU | 2761 | O | HOH | | 41 | 2167 | 2203 | 2038 | 386 | 92 | −259 | O |
| HETATM | 2762 | O | HOH | | 42 | −0.489 | −15.669 | −18.884 | 1.00 | 16.44 | | O |
| ANISOU | 2762 | O | HOH | | 42 | 1883 | 2448 | 1916 | 254 | −44 | 159 | O |
| HETATM | 2763 | O | HOH | | 43 | 23.531 | 9.945 | 21.371 | 1.00 | 15.35 | | O |
| ANISOU | 2763 | O | HOH | | 43 | 1823 | 1682 | 2328 | −273 | −450 | −323 | O |
| HETATM | 2764 | O | HOH | | 44 | −0.481 | 26.908 | 32.216 | 1.00 | 18.68 | | O |
| ANISOU | 2764 | O | HOH | | 44 | 2660 | 2624 | 1813 | 568 | −1011 | −284 | O |
| HETATM | 2765 | O | HOH | | 45 | −10.390 | 1.117 | −14.189 | 1.00 | 17.80 | | O |
| ANISOU | 2765 | O | HOH | | 45 | 1662 | 2775 | 2325 | 484 | 12 | −32 | O |
| HETATM | 2766 | O | HOH | | 46 | 2.164 | 26.665 | 2.603 | 1.00 | 19.78 | | O |
| ANISOU | 2766 | O | HOH | | 46 | 2734 | 1987 | 2795 | 300 | −300 | 641 | O |
| HETATM | 2767 | O | HOH | | 47 | −6.653 | 24.266 | 29.685 | 1.00 | 17.21 | | O |
| ANISOU | 2767 | O | HOH | | 47 | 1629 | 2868 | 2041 | 485 | 143 | 830 | O |
| HETATM | 2768 | O | HOH | | 48 | −4.027 | 24.444 | 5.337 | 1.00 | 15.03 | | O |
| ANISOU | 2768 | O | HOH | | 48 | 1467 | 1961 | 2284 | −73 | −413 | 431 | O |
| HETATM | 2769 | O | HOH | | 49 | 16.261 | 13.320 | 15.193 | 1.00 | 18.49 | | O |
| ANISOU | 2769 | O | HOH | | 49 | 1124 | 2731 | 3173 | −261 | −164 | −198 | O |
| HETATM | 2770 | O | HOH | | 50 | −2.838 | 12.383 | 8.459 | 1.00 | 20.06 | | O |
| ANISOU | 2770 | O | HOH | | 50 | 1976 | 1678 | 3968 | −265 | −125 | 481 | O |
| HETATM | 2771 | O | HOH | | 51 | −1.035 | −6.930 | 7.341 | 1.00 | 18.55 | | O |
| ANISOU | 2771 | O | HOH | | 51 | 2150 | 3136 | 1761 | −28 | 405 | −210 | O |
| HETATM | 2772 | O | HOH | | 52 | −12.100 | 2.004 | −9.492 | 1.00 | 24.25 | | O |
| ANISOU | 2772 | O | HOH | | 52 | 2754 | 3693 | 2767 | 387 | −400 | −581 | O |
| HETATM | 2773 | O | HOH | | 53 | −12.421 | 25.351 | 16.547 | 1.00 | 20.50 | | O |
| ANISOU | 2773 | O | HOH | | 53 | 2124 | 2766 | 2900 | −5 | −386 | 489 | O |
| HETATM | 2774 | O | HOH | | 54 | 5.169 | 3.457 | −4.670 | 1.00 | 21.06 | | O |
| ANISOU | 2774 | O | HOH | | 54 | 2275 | 3392 | 2337 | −497 | −425 | 1263 | O |
| HETATM | 2775 | O | HOH | | 55 | 9.141 | −12.138 | −23.341 | 1.00 | 17.54 | | O |
| ANISOU | 2775 | O | HOH | | 55 | 1438 | 2073 | 3151 | −42 | 238 | −110 | O |
| HETATM | 2776 | O | HOH | | 56 | 10.866 | 3.461 | 4.728 | 1.00 | 16.38 | | O |
| ANISOU | 2776 | O | HOH | | 56 | 1950 | 2323 | 1951 | 9 | −164 | −351 | O |
| HETATM | 2777 | O | HOH | | 57 | 7.398 | −21.370 | −23.752 | 1.00 | 23.94 | | O |
| ANISOU | 2777 | O | HOH | | 57 | 3938 | 2412 | 2744 | 1689 | −167 | −376 | O |
| HETATM | 2778 | O | HOH | | 58 | −0.758 | −8.430 | −31.143 | 1.00 | 25.12 | | O |
| ANISOU | 2778 | O | HOH | | 58 | 1539 | 3920 | 4086 | −446 | −304 | 928 | O |
| HETATM | 2779 | O | HOH | | 59 | −5.675 | −8.176 | −20.235 | 1.00 | 15.37 | | O |
| ANISOU | 2779 | O | HOH | | 59 | 1232 | 2519 | 2089 | −95 | −289 | −614 | O |
| HETATM | 2780 | O | HOH | | 60 | 14.907 | 0.372 | −19.451 | 1.00 | 23.65 | | O |
| ANISOU | 2780 | O | HOH | | 60 | 2033 | 3110 | 3842 | −225 | −24 | 753 | O |
| HETATM | 2781 | O | HOH | | 61 | 17.293 | −2.016 | 5.581 | 1.00 | 19.06 | | O |
| ANISOU | 2781 | O | HOH | | 61 | 1505 | 2609 | 3127 | −65 | −985 | −603 | O |
| HETATM | 2782 | O | HOH | | 62 | −13.834 | −2.499 | −7.963 | 1.00 | 21.14 | | O |
| ANISOU | 2782 | O | HOH | | 62 | 1505 | 3938 | 2588 | −51 | −488 | −756 | O |
| HETATM | 2783 | O | HOH | | 63 | −10.790 | 19.836 | 14.107 | 1.00 | 22.08 | | O |
| ANISOU | 2783 | O | HOH | | 63 | 2425 | 3469 | 2494 | 1350 | −238 | 37 | O |
| HETATM | 2784 | O | HOH | | 64 | 16.350 | −3.360 | −19.909 | 1.00 | 23.17 | | O |
| ANISOU | 2784 | O | HOH | | 64 | 2115 | 3407 | 3280 | 216 | 1113 | 171 | O |
| HETATM | 2785 | O | HOH | | 65 | 14.332 | 16.551 | 11.082 | 1.00 | 24.65 | | O |
| ANISOU | 2785 | O | HOH | | 65 | 1876 | 3022 | 4468 | 238 | −249 | −1032 | O |
| HETATM | 2786 | O | HOH | | 66 | −3.086 | −10.168 | −22.616 | 1.00 | 19.84 | | O |
| ANISOU | 2786 | O | HOH | | 66 | 1475 | 3230 | 2834 | −324 | −15 | −637 | O |
| HETATM | 2787 | O | HOH | | 67 | 13.316 | 0.581 | −13.373 | 1.00 | 20.33 | | O |
| ANISOU | 2787 | O | HOH | | 67 | 2384 | 2603 | 2737 | −722 | 232 | 105 | O |
| HETATM | 2788 | O | HOH | | 68 | −15.595 | 19.427 | 21.612 | 1.00 | 21.55 | | O |
| ANISOU | 2788 | O | HOH | | 68 | 2154 | 3194 | 2839 | 205 | 92 | −904 | O |
| HETATM | 2789 | O | HOH | | 69 | 7.185 | −10.887 | 5.562 | 1.00 | 22.08 | | O |
| ANISOU | 2789 | O | HOH | | 69 | 2095 | 3684 | 2609 | −602 | 106 | −569 | O |
| HETATM | 2790 | O | HOH | | 70 | 2.579 | −14.856 | 4.225 | 1.00 | 22.79 | | O |
| ANISOU | 2790 | O | HOH | | 70 | 2308 | 2864 | 3486 | −582 | −173 | 398 | O |
| HETATM | 2791 | O | HOH | | 71 | −4.077 | −12.484 | −19.244 | 1.00 | 32.67 | | O |
| ANISOU | 2791 | O | HOH | | 71 | 4684 | 4111 | 3619 | −1252 | −237 | −2036 | O |
| HETATM | 2792 | O | HOH | | 72 | 11.093 | −15.710 | −10.280 | 1.00 | 20.37 | | O |
| ANISOU | 2792 | O | HOH | | 72 | 1872 | 2635 | 3232 | −499 | −785 | 482 | O |
| HETATM | 2793 | O | HOH | | 73 | 18.025 | 11.927 | 13.626 | 1.00 | 22.82 | | O |
| ANISOU | 2793 | O | HOH | | 73 | 2716 | 2335 | 3620 | −737 | 23 | −651 | O |
| HETATM | 2794 | O | HOH | | 74 | 6.250 | −2.147 | 13.195 | 1.00 | 16.39 | | O |
| ANISOU | 2794 | O | HOH | | 74 | 1978 | 1917 | 2331 | 131 | 141 | 132 | O |
| HETATM | 2795 | O | HOH | | 75 | 6.016 | 2.298 | 25.972 | 1.00 | 24.81 | | O |
| ANISOU | 2795 | O | HOH | | 75 | 3604 | 3020 | 2804 | 913 | −264 | 960 | O |
| HETATM | 2796 | O | HOH | | 76 | 2.839 | −13.437 | −26.745 | 1.00 | 21.07 | | O |
| ANISOU | 2796 | O | HOH | | 76 | 2927 | 2884 | 2194 | −1138 | 177 | −797 | O |
| HETATM | 2797 | O | HOH | | 77 | 4.431 | 18.972 | 3.550 | 1.00 | 23.38 | | O |
| ANISOU | 2797 | O | HOH | | 77 | 2787 | 3217 | 2881 | −1165 | 349 | 463 | O |
| HETATM | 2798 | O | HOH | | 78 | −20.608 | −14.075 | −10.025 | 1.00 | 22.94 | | O |
| ANISOU | 2798 | O | HOH | | 78 | 1860 | 4214 | 2643 | 159 | 655 | −467 | O |
| HETATM | 2799 | O | HOH | | 79 | −3.637 | 8.081 | −9.527 | 1.00 | 20.98 | | O |
| ANISOU | 2799 | O | HOH | | 79 | 1906 | 2300 | 3766 | 153 | −715 | 188 | O |
| HETATM | 2800 | O | HOH | | 80 | 25.392 | 5.053 | 26.986 | 1.00 | 18.60 | | O |
| ANISOU | 2800 | O | HOH | | 80 | 1746 | 3002 | 2319 | −201 | 67 | 52 | O |
| HETATM | 2801 | O | HOH | | 81 | −3.811 | −6.245 | 7.313 | 1.00 | 17.57 | | O |
| ANISOU | 2801 | O | HOH | | 81 | 1909 | 2797 | 1971 | −105 | 251 | 30 | O |
| HETATM | 2802 | O | HOH | | 82 | 8.066 | 22.858 | 24.779 | 1.00 | 17.54 | | O |
| ANISOU | 2802 | O | HOH | | 82 | 1199 | 2246 | 3220 | −266 | −630 | 26 | O |
| HETATM | 2803 | O | HOH | | 83 | −2.578 | 14.161 | 5.094 | 1.00 | 28.82 | | O |
| ANISOU | 2803 | O | HOH | | 83 | 3412 | 3722 | 3816 | −14 | −1292 | 40 | O |
| HETATM | 2804 | O | HOH | | 84 | −3.653 | −16.601 | −1.468 | 1.00 | 19.52 | | O |
| ANISOU | 2804 | O | HOH | | 84 | 2706 | 1866 | 2844 | −579 | 78 | 521 | O |
| HETATM | 2805 | O | HOH | | 85 | −5.517 | 2.329 | 5.451 | 1.00 | 28.91 | | O |
| ANISOU | 2805 | O | HOH | | 85 | 2319 | 5416 | 3250 | 536 | 1008 | −1322 | O |
| HETATM | 2806 | O | HOH | | 86 | 13.631 | −7.083 | −6.372 | 1.00 | 29.87 | | O |
| ANISOU | 2806 | O | HOH | | 86 | 2265 | 6053 | 3033 | −255 | 272 | −65 | O |
| HETATM | 2807 | O | HOH | | 87 | 5.367 | 8.648 | −16.546 | 1.00 | 21.78 | | O |
| ANISOU | 2807 | O | HOH | | 87 | 2916 | 2451 | 2909 | −323 | −261 | 668 | O |
| HETATM | 2808 | O | HOH | | 88 | −9.312 | 27.415 | 23.479 | 1.00 | 27.15 | | O |
| ANISOU | 2808 | O | HOH | | 88 | 3017 | 3528 | 3770 | −216 | 208 | 329 | O |
| HETATM | 2809 | O | HOH | | 89 | 17.207 | −18.417 | 4.547 | 1.00 | 18.54 | | O |
| ANISOU | 2809 | O | HOH | | 89 | 1920 | 2749 | 2375 | 251 | −65 | 786 | O |
| HETATM | 2810 | O | HOH | | 90 | 7.892 | 2.675 | 5.904 | 1.00 | 25.01 | | O |
| ANISOU | 2810 | O | HOH | | 90 | 1567 | 3991 | 3944 | −694 | 480 | −2175 | O |
| HETATM | 2811 | O | HOH | | 91 | 4.914 | 11.012 | 28.442 | 1.00 | 18.97 | | O |
| ANISOU | 2811 | O | HOH | | 91 | 2353 | 2644 | 2210 | −436 | −894 | 198 | O |
| HETATM | 2812 | O | HOH | | 92 | −2.263 | 22.927 | 36.654 | 1.00 | 25.65 | | O |
| ANISOU | 2812 | O | HOH | | 92 | 2720 | 4187 | 2840 | −31 | 122 | −1125 | O |
| HETATM | 2813 | O | HOH | | 93 | −10.938 | 14.269 | 12.202 | 1.00 | 27.36 | | O |
| ANISOU | 2813 | O | HOH | | 93 | 1644 | 4892 | 3858 | −1184 | −514 | −240 | O |
| HETATM | 2814 | O | HOH | | 94 | −7.470 | 21.500 | 33.892 | 1.00 | 27.56 | | O |
| ANISOU | 2814 | O | HOH | | 94 | 2033 | 6169 | 2270 | 1919 | −6 | −1208 | O |
| HETATM | 2815 | O | HOH | | 95 | −5.997 | 24.245 | 11.064 | 1.00 | 21.84 | | O |
| ANISOU | 2815 | O | HOH | | 95 | 1747 | 3468 | 3084 | −190 | 758 | −314 | O |
| HETATM | 2816 | O | HOH | | 96 | 1.288 | 31.330 | 17.124 | 1.00 | 30.00 | | O |
| ANISOU | 2816 | O | HOH | | 96 | 5110 | 2189 | 4099 | −336 | 484 | −595 | O |
| HETATM | 2817 | O | HOH | | 97 | −5.810 | 12.973 | 9.359 | 1.00 | 26.00 | | O |
| ANISOU | 2817 | O | HOH | | 97 | 3106 | 2945 | 3827 | 630 | −2335 | −1572 | O |
| HETATM | 2818 | O | HOH | | 98 | 15.604 | 11.256 | 7.663 | 1.00 | 23.46 | | O |
| ANISOU | 2818 | O | HOH | | 98 | 2918 | 3075 | 2919 | 273 | 195 | −326 | O |
| HETATM | 2819 | O | HOH | | 99 | 10.512 | 9.839 | −29.325 | 1.00 | 26.81 | | O |
| ANISOU | 2819 | O | HOH | | 99 | 2486 | 3657 | 4044 | −465 | −1013 | −540 | O |
| HETATM | 2820 | O | HOH | | 100 | −13.652 | −3.276 | −14.265 | 1.00 | 23.60 | | O |
| ANISOU | 2820 | O | HOH | | 100 | 2649 | 3147 | 3169 | 13 | −629 | −711 | O |
| HETATM | 2821 | O | HOH | | 101 | 6.479 | 24.867 | 0.973 | 1.00 | 26.06 | | O |
| ANISOU | 2821 | O | HOH | | 101 | 4159 | 2712 | 3032 | 419 | 217 | −387 | O |
| HETATM | 2822 | O | HOH | | 102 | 5.566 | 24.333 | 27.524 | 1.00 | 22.78 | | O |
| ANISOU | 2822 | O | HOH | | 102 | 1387 | 5319 | 1949 | 69 | −69 | −158 | O |
| HETATM | 2823 | O | HOH | | 103 | −17.240 | −5.163 | −6.960 | 1.00 | 26.53 | | O |
| ANISOU | 2823 | O | HOH | | 103 | 4261 | 3299 | 2520 | −918 | −712 | 915 | O |
| HETATM | 2824 | O | HOH | | 104 | 4.695 | −19.132 | −24.426 | 1.00 | 25.47 | | O |
| ANISOU | 2824 | O | HOH | | 104 | 2840 | 3101 | 3737 | 402 | −86 | −110 | O |
| HETATM | 2825 | O | HOH | | 105 | −8.743 | 11.174 | 28.450 | 1.00 | 23.35 | | O |
| ANISOU | 2825 | O | HOH | | 105 | 2221 | 3449 | 3203 | −633 | −867 | 872 | O |
| HETATM | 2826 | O | HOH | | 106 | 5.234 | 28.798 | 20.929 | 1.00 | 29.06 | | O |
| ANISOU | 2826 | O | HOH | | 106 | 3944 | 2678 | 4419 | −347 | 693 | −540 | O |
| HETATM | 2827 | O | HOH | | 107 | 6.350 | 20.375 | 28.446 | 1.00 | 24.01 | | O |
| ANISOU | 2827 | O | HOH | | 107 | 2842 | 3883 | 2395 | −1259 | −1233 | −653 | O |
| HETATM | 2828 | O | HOH | | 108 | 19.732 | 11.700 | 17.147 | 1.00 | 27.60 | | O |
| ANISOU | 2828 | O | HOH | | 108 | 4596 | 1929 | 3963 | 755 | −1827 | −526 | O |
| HETATM | 2829 | O | HOH | | 109 | −6.592 | 0.285 | 7.538 | 1.00 | 28.17 | | O |
| ANISOU | 2829 | O | HOH | | 109 | 2310 | 4141 | 4253 | −587 | −720 | −256 | O |
| HETATM | 2830 | O | HOH | | 110 | 17.522 | −7.648 | −13.586 | 1.00 | 27.42 | | O |
| ANISOU | 2830 | O | HOH | | 110 | 3100 | 3543 | 3774 | 1069 | 1328 | 286 | O |
| HETATM | 2831 | O | HOH | | 111 | 29.382 | −5.440 | 15.607 | 1.00 | 30.64 | | O |
| ANISOU | 2831 | O | HOH | | 111 | 3533 | 4132 | 3976 | 783 | −122 | 369 | O |
| HETATM | 2832 | O | HOH | | 112 | 14.830 | −11.730 | −20.641 | 1.00 | 23.79 | | O |
| ANISOU | 2832 | O | HOH | | 112 | 2690 | 2890 | 3459 | 190 | 465 | 300 | O |
| HETATM | 2833 | O | HOH | | 113 | 17.693 | −3.008 | −12.077 | 1.00 | 27.82 | | O |
| ANISOU | 2833 | O | HOH | | 113 | 2096 | 5037 | 3437 | −724 | 492 | −1148 | O |
| HETATM | 2834 | O | HOH | | 114 | 4.986 | 7.622 | −4.402 | 1.00 | 46.16 | | O |
| ANISOU | 2834 | O | HOH | | 114 | 5081 | 5557 | 6902 | −1276 | −194 | 453 | O |
| HETATM | 2835 | O | HOH | | 115 | 10.188 | 2.689 | −13.884 | 1.00 | 44.86 | | O |
| ANISOU | 2835 | O | HOH | | 115 | 4800 | 5041 | 7204 | −2687 | 1288 | 85 | O |
| HETATM | 2836 | O | HOH | | 116 | −15.177 | 17.227 | 16.806 | 1.00 | 31.99 | | O |
| ANISOU | 2836 | O | HOH | | 116 | 2926 | 4004 | 5223 | 1141 | −2371 | −1614 | O |
| HETATM | 2837 | O | HOH | | 117 | 2.454 | −8.568 | 7.910 | 1.00 | 30.62 | | O |
| ANISOU | 2837 | O | HOH | | 117 | 2914 | 5280 | 3439 | −2092 | 168 | 362 | O |
| HETATM | 2838 | O | HOH | | 118 | −0.021 | −13.881 | −20.935 | 1.00 | 23.10 | | O |
| ANISOU | 2838 | O | HOH | | 118 | 2202 | 3992 | 2584 | −349 | 498 | 99 | O |
| HETATM | 2839 | O | HOH | | 119 | −18.626 | −11.390 | −8.903 | 1.00 | 20.94 | | O |
| ANISOU | 2839 | O | HOH | | 119 | 2908 | 3636 | 1414 | −744 | 20 | −370 | O |
| HETATM | 2840 | O | HOH | | 120 | −5.857 | 23.100 | 7.416 | 1.00 | 31.41 | | O |
| ANISOU | 2840 | O | HOH | | 120 | 2691 | 5660 | 3584 | 525 | −1294 | −1260 | O |
| HETATM | 2841 | O | HOH | | 121 | −2.655 | −2.348 | −24.596 | 1.00 | 31.83 | | O |
| ANISOU | 2841 | O | HOH | | 121 | 4217 | 5691 | 2187 | −86 | −541 | 627 | O |
| HETATM | 2842 | O | HOH | | 122 | −18.526 | −17.197 | −10.078 | 1.00 | 27.24 | | O |
| ANISOU | 2842 | O | HOH | | 122 | 4524 | 3401 | 2424 | −213 | 728 | 520 | O |
| HETATM | 2843 | O | HOH | | 123 | −12.046 | 13.976 | 16.467 | 1.00 | 26.28 | | O |
| ANISOU | 2843 | O | HOH | | 123 | 1987 | 3651 | 4346 | −79 | 203 | −334 | O |
| HETATM | 2844 | O | HOH | | 124 | −22.934 | −16.612 | −4.201 | 1.00 | 19.46 | | O |
| ANISOU | 2844 | O | HOH | | 124 | 2073 | 1863 | 3457 | −61 | −243 | 242 | O |
| HETATM | 2845 | O | HOH | | 125 | 14.884 | 8.724 | 7.401 | 1.00 | 23.65 | | O |
| ANISOU | 2845 | O | HOH | | 125 | 2899 | 3177 | 2909 | 914 | −513 | 378 | O |
| HETATM | 2846 | O | HOH | | 126 | 17.967 | −6.290 | −16.222 | 1.00 | 38.30 | | O |
| ANISOU | 2846 | O | HOH | | 126 | 2044 | 6505 | 6002 | −932 | 897 | −2776 | O |
| HETATM | 2847 | O | HOH | | 127 | 15.521 | −13.936 | −12.033 | 1.00 | 28.63 | | O |
| ANISOU | 2847 | O | HOH | | 127 | 2939 | 3689 | 4249 | 964 | −640 | 156 | O |
| HETATM | 2848 | O | HOH | | 128 | −16.559 | 14.939 | 22.502 | 1.00 | 31.69 | | O |
| ANISOU | 2848 | O | HOH | | 128 | 2058 | 4935 | 5047 | 1614 | 490 | −348 | O |
| HETATM | 2849 | O | HOH | | 129 | 5.551 | 2.828 | −0.540 | 1.00 | 22.83 | | O |
| ANISOU | 2849 | O | HOH | | 129 | 2995 | 2713 | 2967 | −640 | −449 | 214 | O |
| HETATM | 2850 | O | HOH | | 130 | −21.554 | −3.638 | −2.547 | 1.00 | 28.41 | | O |
| ANISOU | 2850 | O | HOH | | 130 | 4057 | 4318 | 2419 | −1185 | 299 | 364 | O |
| HETATM | 2851 | O | HOH | | 131 | 17.055 | 11.852 | 24.355 | 1.00 | 30.49 | | O |
| ANISOU | 2851 | O | HOH | | 131 | 2823 | 3435 | 5326 | 203 | 661 | −2009 | O |
| HETATM | 2852 | O | HOH | | 132 | −10.787 | 18.232 | 11.556 | 1.00 | 25.96 | | O |
| ANISOU | 2852 | O | HOH | | 132 | 1946 | 4413 | 3503 | 524 | 102 | 382 | O |
| HETATM | 2853 | O | HOH | | 133 | −2.679 | −15.804 | −10.344 | 1.00 | 18.22 | | O |
| ANISOU | 2853 | O | HOH | | 133 | 2144 | 2491 | 2287 | −420 | −872 | −572 | O |
| HETATM | 2854 | O | HOH | | 134 | 15.865 | 11.104 | 22.278 | 1.00 | 28.82 | | O |
| ANISOU | 2854 | O | HOH | | 134 | 1463 | 4570 | 4916 | 1227 | −143 | 134 | O |
| HETATM | 2855 | O | HOH | | 135 | −3.949 | 8.821 | 32.302 | 1.00 | 36.85 | | O |
| ANISOU | 2855 | O | HOH | | 135 | 5464 | 4648 | 3891 | 548 | −652 | 1016 | O |
| HETATM | 2856 | O | HOH | | 136 | 10.661 | 0.544 | 5.065 | 1.00 | 29.75 | | O |
| ANISOU | 2856 | O | HOH | | 136 | 2431 | 4101 | 4770 | −835 | 808 | −544 | O |
| HETATM | 2857 | O | HOH | | 137 | −12.495 | 3.023 | −0.778 | 1.00 | 22.94 | | O |
| ANISOU | 2857 | O | HOH | | 137 | 2730 | 3284 | 2703 | −569 | −377 | 222 | O |
| HETATM | 2858 | O | HOH | | 138 | 0.571 | 10.166 | −9.912 | 1.00 | 52.04 | | O |
| ANISOU | 2858 | O | HOH | | 138 | 5989 | 5897 | 7887 | 3495 | −867 | −1840 | O |
| HETATM | 2859 | O | HOH | | 139 | 2.384 | 21.580 | 30.274 | 1.00 | 25.64 | | O |
| ANISOU | 2859 | O | HOH | | 139 | 2719 | 3158 | 3866 | −50 | −237 | −28 | O |
| HETATM | 2860 | O | HOH | | 140 | 7.355 | 0.222 | 24.925 | 1.00 | 30.69 | | O |
| ANISOU | 2860 | O | HOH | | 140 | 4868 | 3587 | 3206 | 575 | 196 | 856 | O |
| HETATM | 2861 | O | HOH | | 141 | 22.983 | 11.070 | 26.778 | 1.00 | 31.02 | | O |
| ANISOU | 2861 | O | HOH | | 141 | 2459 | 4193 | 5135 | 1301 | 112 | −548 | O |
| HETATM | 2862 | O | HOH | | 142 | 16.108 | 19.029 | 18.039 | 1.00 | 22.91 | | O |
| ANISOU | 2862 | O | HOH | | 142 | 2094 | 3873 | 2736 | −777 | −122 | −1043 | O |
| HETATM | 2863 | O | HOH | | 143 | 2.833 | 30.925 | 8.128 | 1.00 | 36.53 | | O |
| ANISOU | 2863 | O | HOH | | 143 | 6034 | 4416 | 3428 | −1659 | −1242 | 1883 | O |
| HETATM | 2864 | O | HOH | | 144 | −9.525 | 9.532 | 9.222 | 1.00 | 31.12 | | O |
| ANISOU | 2864 | O | HOH | | 144 | 3303 | 3556 | 4966 | −155 | −409 | −1921 | O |
| HETATM | 2865 | O | HOH | | 145 | 0.183 | 34.181 | 10.411 | 1.00 | 48.59 | | O |
| ANISOU | 2865 | O | HOH | | 145 | 7987 | 3568 | 6905 | −1242 | −339 | 2882 | O |
| HETATM | 2866 | O | HOH | | 146 | −9.731 | −11.349 | −15.789 | 1.00 | 32.31 | | O |
| ANISOU | 2866 | O | HOH | | 146 | 3513 | 3629 | 5135 | 329 | −917 | −741 | O |
| HETATM | 2867 | O | HOH | | 147 | 11.970 | −10.646 | −19.183 | 1.00 | 23.87 | | O |
| ANISOU | 2867 | O | HOH | | 147 | 2174 | 3694 | 3200 | 220 | 635 | 257 | O |
| HETATM | 2868 | O | HOH | | 148 | −0.255 | −2.271 | 22.408 | 1.00 | 38.24 | | O |
| ANISOU | 2868 | O | HOH | | 148 | 4764 | 5086 | 4681 | −1031 | 1380 | −215 | O |
| HETATM | 2869 | O | HOH | | 149 | 8.911 | 0.034 | 6.997 | 1.00 | 26.14 | | O |
| ANISOU | 2869 | O | HOH | | 149 | 2702 | 3607 | 3623 | −166 | 99 | −540 | O |
| HETATM | 2870 | O | HOH | | 150 | 3.699 | 25.165 | 0.913 | 1.00 | 21.53 | | O |
| ANISOU | 2870 | O | HOH | | 150 | 3301 | 2067 | 2813 | 352 | −85 | 122 | O |
| HETATM | 2871 | O | HOH | | 151 | 13.592 | −6.774 | −8.775 | 1.00 | 24.94 | | O |
| ANISOU | 2871 | O | HOH | | 151 | 2747 | 3506 | 3224 | 104 | −109 | −80 | O |
| HETATM | 2872 | O | HOH | | 152 | 16.549 | −7.361 | −10.098 | 1.00 | 24.51 | | O |
| ANISOU | 2872 | O | HOH | | 152 | 3173 | 2529 | 3608 | −485 | −597 | 322 | O |
| HETATM | 2873 | O | HOH | | 153 | −11.050 | 12.675 | 19.402 | 1.00 | 28.86 | | O |
| ANISOU | 2873 | O | HOH | | 153 | 3813 | 3513 | 3640 | −555 | 367 | −616 | O |
| HETATM | 2874 | O | HOH | | 154 | 14.333 | −3.582 | −8.917 | 1.00 | 27.73 | | O |
| ANISOU | 2874 | O | HOH | | 154 | 1881 | 6030 | 2624 | −657 | 104 | 201 | O |
| HETATM | 2875 | O | HOH | | 155 | 19.398 | −4.896 | 8.666 | 1.00 | 68.86 | | O |
| ANISOU | 2875 | O | HOH | | 155 | 8395 | 8879 | 8891 | 91 | 52 | −266 | O |
| HETATM | 2876 | O | HOH | | 156 | 13.944 | −4.520 | −3.950 | 1.00 | 27.85 | | O |
| ANISOU | 2876 | O | HOH | | 156 | 3260 | 3488 | 3833 | 375 | −170 | 248 | O |
| HETATM | 2877 | O | HOH | | 157 | −11.066 | 10.303 | 18.727 | 1.00 | 32.51 | | O |
| ANISOU | 2877 | O | HOH | | 157 | 2321 | 4084 | 5947 | −480 | 395 | −744 | O |
| HETATM | 2878 | O | HOH | | 158 | −2.296 | 21.295 | 1.498 | 1.00 | 33.21 | | O |
| ANISOU | 2878 | O | HOH | | 158 | 3965 | 6061 | 2593 | −425 | 118 | −457 | O |
| HETATM | 2879 | O | HOH | | 159 | −0.505 | 8.159 | 30.508 | 1.00 | 28.77 | | O |
| ANISOU | 2879 | O | HOH | | 159 | 4019 | 3554 | 3358 | −1637 | −432 | 773 | O |
| HETATM | 2880 | O | HOH | | 160 | −8.365 | 2.127 | 7.748 | 1.00 | 29.86 | | O |
| ANISOU | 2880 | O | HOH | | 160 | 3105 | 3989 | 4251 | −23 | −1125 | 436 | O |
| HETATM | 2881 | O | HOH | | 161 | 13.275 | 0.468 | −7.591 | 1.00 | 35.94 | | O |
| ANISOU | 2881 | O | HOH | | 161 | 1671 | 5109 | 6877 | 557 | −1929 | −2078 | O |
| HETATM | 2882 | O | HOH | | 162 | 6.727 | −17.443 | 0.735 | 1.00 | 30.67 | | O |
| ANISOU | 2882 | O | HOH | | 162 | 2124 | 4487 | 5043 | −347 | −2073 | 932 | O |
| HETATM | 2883 | O | HOH | | 163 | −2.086 | 10.057 | −9.121 | 1.00 | 33.09 | | O |
| ANISOU | 2883 | O | HOH | | 163 | 4077 | 3611 | 4886 | 443 | 22 | −551 | O |
| HETATM | 2884 | O | HOH | | 164 | 4.468 | 13.060 | −10.655 | 1.00 | 45.12 | | O |
| ANISOU | 2884 | O | HOH | | 164 | 6169 | 5187 | 5787 | 859 | 680 | −1588 | O |
| HETATM | 2885 | O | HOH | | 165 | 12.066 | −5.445 | −24.053 | 1.00 | 24.64 | | O |
| ANISOU | 2885 | O | HOH | | 165 | 3351 | 3221 | 2790 | 656 | −199 | 47 | O |
| HETATM | 2886 | O | HOH | | 166 | 19.341 | −0.477 | −16.807 | 1.00 | 38.17 | | O |
| ANISOU | 2886 | O | HOH | | 166 | 3932 | 5601 | 4970 | −819 | 717 | 596 | O |
| HETATM | 2887 | O | HOH | | 167 | −22.527 | −18.245 | −1.442 | 1.00 | 32.21 | | O |
| ANISOU | 2887 | O | HOH | | 167 | 4597 | 4054 | 3587 | −377 | −452 | −768 | O |
| HETATM | 2888 | O | HOH | | 168 | 20.874 | 9.974 | 29.508 | 1.00 | 26.50 | | O |
| ANISOU | 2888 | O | HOH | | 168 | 4941 | 2222 | 2904 | 623 | −773 | 126 | O |
| HETATM | 2889 | O | HOH | | 169 | −4.475 | 35.026 | 18.875 | 1.00 | 30.06 | | O |
| ANISOU | 2889 | O | HOH | | 169 | 4988 | 1960 | 4472 | 329 | 1346 | −23 | O |
| HETATM | 2890 | O | HOH | | 170 | −0.787 | 10.092 | −21.877 | 1.00 | 31.24 | | O |
| ANISOU | 2890 | O | HOH | | 170 | 4276 | 3709 | 3884 | −838 | −597 | 1591 | O |
| HETATM | 2891 | O | HOH | | 171 | 10.373 | 22.480 | 1.995 | 1.00 | 27.65 | | O |
| ANISOU | 2891 | O | HOH | | 171 | 2862 | 3928 | 3713 | 9 | 1044 | 1280 | O |
| HETATM | 2892 | O | HOH | | 172 | 18.442 | 5.116 | −0.772 | 1.00 | 57.07 | | O |
| ANISOU | 2892 | O | HOH | | 172 | 7556 | 7104 | 7023 | −617 | 693 | 84 | O |
| HETATM | 2893 | O | HOH | | 173 | −10.889 | −0.452 | −18.914 | 1.00 | 34.73 | | O |
| ANISOU | 2893 | O | HOH | | 173 | 2509 | 5933 | 4756 | 1160 | 514 | 1842 | O |
| HETATM | 2894 | O | HOH | | 174 | −9.211 | −1.983 | 26.266 | 1.00 | 31.11 | | O |
| ANISOU | 2894 | O | HOH | | 174 | 4873 | 2759 | 4187 | −285 | −835 | −48 | O |
| HETATM | 2895 | O | HOH | | 175 | 2.630 | −18.976 | −8.130 | 1.00 | 30.42 | | O |
| ANISOU | 2895 | O | HOH | | 175 | 3643 | 4245 | 3671 | 1615 | −759 | −1329 | O |
| HETATM | 2896 | O | HOH | | 176 | −15.604 | −1.901 | −6.076 | 1.00 | 37.16 | | O |
| ANISOU | 2896 | O | HOH | | 176 | 4897 | 4457 | 4766 | 732 | −1597 | 324 | O |
| HETATM | 2897 | O | HOH | | 177 | 8.326 | 2.665 | −5.538 | 1.00 | 28.13 | | O |
| ANISOU | 2897 | O | HOH | | 177 | 3692 | 4482 | 2513 | −2555 | −106 | −299 | O |
| HETATM | 2898 | O | HOH | | 178 | 9.814 | −17.000 | −22.434 | 1.00 | 33.28 | | O |
| ANISOU | 2898 | O | HOH | | 178 | 3647 | 3692 | 5306 | −1347 | 1965 | −1786 | O |
| HETATM | 2899 | O | HOH | | 179 | 17.533 | −10.496 | 4.438 | 1.00 | 28.93 | | O |
| ANISOU | 2899 | O | HOH | | 179 | 2140 | 6198 | 2653 | −903 | −474 | 325 | O |
| HETATM | 2900 | O | HOH | | 180 | −16.030 | 0.882 | −3.090 | 1.00 | 28.36 | | O |
| ANISOU | 2900 | O | HOH | | 180 | 3043 | 4690 | 3041 | −303 | −724 | 409 | O |
| HETATM | 2901 | O | HOH | | 181 | −12.321 | 29.033 | 19.835 | 1.00 | 27.87 | | O |
| ANISOU | 2901 | O | HOH | | 181 | 2774 | 2881 | 4933 | 1110 | 524 | −301 | O |
| HETATM | 2902 | O | HOH | | 182 | −14.711 | −11.839 | −9.764 | 1.00 | 30.38 | | O |
| ANISOU | 2902 | O | HOH | | 182 | 3463 | 5383 | 2698 | 484 | 810 | −486 | O |
| HETATM | 2903 | O | HOH | | 183 | −15.453 | −8.172 | −8.084 | 1.00 | 33.43 | | O |
| ANISOU | 2903 | O | HOH | | 183 | 4434 | 4892 | 3375 | −1158 | 1818 | −311 | O |
| HETATM | 2904 | O | HOH | | 184 | 2.195 | 36.674 | 11.652 | 1.00 | 42.25 | | O |
| ANISOU | 2904 | O | HOH | | 184 | 6132 | 5070 | 4851 | −1060 | −1093 | 1751 | O |
| HETATM | 2905 | O | HOH | | 185 | 15.024 | 22.116 | 10.091 | 1.00 | 35.06 | | O |
| ANISOU | 2905 | O | HOH | | 185 | 2120 | 4619 | 6581 | 88 | −144 | −590 | O |
| HETATM | 2906 | O | HOH | | 186 | 12.274 | 5.475 | −24.863 | 1.00 | 53.16 | | O |
| ANISOU | 2906 | O | HOH | | 186 | 8363 | 7104 | 4732 | −658 | −172 | 1304 | O |
| HETATM | 2907 | O | HOH | | 187 | −3.720 | −16.801 | −17.337 | 1.00 | 33.36 | | O |
| ANISOU | 2907 | O | HOH | | 187 | 2094 | 5375 | 5206 | 320 | −727 | −1310 | O |
| HETATM | 2908 | O | HOH | | 188 | 7.102 | 29.319 | 14.048 | 1.00 | 31.22 | | O |
| ANISOU | 2908 | O | HOH | | 188 | 3237 | 5307 | 3320 | −193 | 647 | 423 | O |
| HETATM | 2909 | O | HOH | | 189 | 4.221 | 9.457 | −25.306 | 1.00 | 29.38 | | O |
| ANISOU | 2909 | O | HOH | | 189 | 3732 | 3529 | 3901 | −1254 | −1095 | −167 | O |
| HETATM | 2910 | O | HOH | | 190 | −13.213 | 3.566 | −3.420 | 1.00 | 25.53 | | O |
| ANISOU | 2910 | O | HOH | | 190 | 2787 | 3816 | 3096 | 428 | −410 | 170 | O |
| HETATM | 2911 | O | HOH | | 191 | −5.008 | −16.339 | 0.843 | 1.00 | 30.17 | | O |
| ANISOU | 2911 | O | HOH | | 191 | 4726 | 2864 | 3871 | −211 | 1139 | −342 | O |
| HETATM | 2912 | O | HOH | | 192 | −7.806 | −15.653 | −15.662 | 1.00 | 35.86 | | O |
| ANISOU | 2912 | O | HOH | | 192 | 2143 | 5376 | 6105 | −733 | −205 | −483 | O |
| HETATM | 2913 | O | HOH | | 193 | 18.364 | 10.088 | 10.288 | 1.00 | 23.65 | | O |
| ANISOU | 2913 | O | HOH | | 193 | 1844 | 3399 | 3741 | 25 | −187 | −125 | O |
| HETATM | 2914 | O | HOH | | 194 | −17.364 | 0.396 | 4.267 | 1.00 | 39.23 | | O |
| ANISOU | 2914 | O | HOH | | 194 | 4639 | 5107 | 5160 | 1278 | 801 | 173 | O |
| HETATM | 2915 | O | HOH | | 195 | 7.715 | 4.773 | 29.121 | 1.00 | 43.37 | | O |
| ANISOU | 2915 | O | HOH | | 195 | 2462 | 6975 | 7041 | 289 | −738 | 1810 | O |
| HETATM | 2916 | O | HOH | | 196 | 9.341 | −19.557 | 3.809 | 1.00 | 33.40 | | O |
| ANISOU | 2916 | O | HOH | | 196 | 4318 | 4235 | 4135 | 889 | −416 | 1434 | O |
| HETATM | 2917 | O | HOH | | 197 | 5.625 | −20.008 | 0.577 | 1.00 | 35.11 | | O |
| ANISOU | 2917 | O | HOH | | 197 | 4908 | 4776 | 3658 | −752 | −1311 | 41 | O |
| HETATM | 2918 | O | HOH | | 198 | −5.915 | 16.736 | 6.049 | 1.00 | 35.11 | | O |
| ANISOU | 2918 | O | HOH | | 198 | 4979 | 5908 | 2453 | 1277 | −1239 | −1137 | O |
| HETATM | 2919 | O | HOH | | 199 | −18.901 | −8.605 | −8.592 | 1.00 | 32.64 | | O |
| ANISOU | 2919 | O | HOH | | 199 | 5131 | 4485 | 2785 | 406 | 283 | −971 | O |
| HETATM | 2920 | O | HOH | | 200 | 0.155 | −3.457 | 15.069 | 1.00 | 35.60 | | O |
| ANISOU | 2920 | O | HOH | | 200 | 4803 | 3146 | 5577 | 699 | −1984 | −1664 | O |
| HETATM | 2921 | O | HOH | | 201 | −5.728 | −15.373 | −17.135 | 1.00 | 39.25 | | O |
| ANISOU | 2921 | O | HOH | | 201 | 4041 | 5834 | 5038 | −261 | −907 | −1384 | O |
| HETATM | 2922 | O | HOH | | 202 | 12.296 | 25.675 | 12.749 | 1.00 | 50.75 | | O |
| ANISOU | 2922 | O | HOH | | 202 | 6187 | 8277 | 4818 | 1662 | −455 | −1397 | O |
| HETATM | 2923 | O | HOH | | 203 | −16.721 | 1.790 | 12.152 | 1.00 | 32.44 | | O |
| ANISOU | 2923 | O | HOH | | 203 | 3019 | 5979 | 3329 | −658 | 340 | −1405 | O |
| HETATM | 2924 | O | HOH | | 204 | −16.660 | 22.446 | 16.153 | 1.00 | 33.75 | | O |
| ANISOU | 2924 | O | HOH | | 204 | 3315 | 4881 | 4628 | −131 | 482 | −664 | O |
| HETATM | 2925 | O | HOH | | 205 | −13.018 | 13.148 | 27.137 | 1.00 | 28.22 | | O |
| ANISOU | 2925 | O | HOH | | 205 | 2470 | 4559 | 3695 | −973 | 700 | 978 | O |
| HETATM | 2926 | O | HOH | | 206 | −14.231 | 12.370 | 30.268 | 1.00 | 34.44 | | O |
| ANISOU | 2926 | O | HOH | | 206 | 2807 | 4695 | 5582 | 1286 | −145 | −1656 | O |
| HETATM | 2927 | O | HOH | | 207 | −15.046 | 16.947 | 22.039 | 1.00 | 28.33 | | O |
| ANISOU | 2927 | O | HOH | | 207 | 2501 | 3599 | 4664 | 428 | 1632 | −186 | O |
| HETATM | 2928 | O | HOH | | 208 | 15.896 | −18.406 | −5.974 | 1.00 | 55.08 | | O |
| ANISOU | 2928 | O | HOH | | 208 | 6963 | 6639 | 7327 | 802 | 499 | 453 | O |
| HETATM | 2929 | O | HOH | | 209 | −5.177 | 31.475 | 28.440 | 1.00 | 32.66 | | O |
| ANISOU | 2929 | O | HOH | | 209 | 2765 | 5080 | 4564 | 1446 | 544 | −1198 | O |
| HETATM | 2930 | O | HOH | | 210 | −12.181 | 5.074 | −13.560 | 1.00 | 54.11 | | O |
| ANISOU | 2930 | O | HOH | | 210 | 5414 | 7078 | 8068 | 605 | 1218 | −1459 | O |
| HETATM | 2931 | O | HOH | | 211 | 1.412 | 19.526 | −2.512 | 1.00 | 39.50 | | O |
| ANISOU | 2931 | O | HOH | | 211 | 4074 | 6986 | 3948 | −1378 | 1418 | −61 | O |
| HETATM | 2932 | O | HOH | | 212 | 5.202 | 10.541 | 2.710 | 1.00 | 31.81 | | O |
| ANISOU | 2932 | O | HOH | | 212 | 4349 | 3454 | 4282 | −330 | 136 | 193 | O |
| HETATM | 2933 | O | HOH | | 213 | 19.035 | −0.193 | 2.638 | 1.00 | 70.08 | | O |
| ANISOU | 2933 | O | HOH | | 213 | 8237 | 9115 | 9275 | −422 | 130 | −823 | O |
| HETATM | 2934 | O | HOH | | 214 | −6.007 | −11.488 | 4.534 | 1.00 | 44.23 | | O |
| ANISOU | 2934 | O | HOH | | 214 | 5357 | 6936 | 4512 | 255 | −843 | 1128 | O |
| HETATM | 2935 | O | HOH | | 215 | −7.475 | −4.285 | −22.591 | 1.00 | 38.89 | | O |
| ANISOU | 2935 | O | HOH | | 215 | 4539 | 6469 | 3770 | −878 | −166 | −3025 | O |
| HETATM | 2936 | O | HOH | | 216 | 12.581 | −6.302 | 9.719 | 1.00 | 30.47 | | O |
| ANISOU | 2936 | O | HOH | | 216 | 4463 | 3437 | 3678 | 256 | 35 | 583 | O |
| HETATM | 2937 | O | HOH | | 217 | −1.794 | −5.613 | −28.310 | 1.00 | 49.56 | | O |
| ANISOU | 2937 | O | HOH | | 217 | 5952 | 9067 | 3811 | −427 | 1095 | 882 | O |
| HETATM | 2938 | O | HOH | | 218 | −11.242 | 13.106 | 5.831 | 1.00 | 45.60 | | O |
| ANISOU | 2938 | O | HOH | | 218 | 6276 | 4955 | 6095 | 302 | −1937 | −663 | O |
| HETATM | 2939 | O | HOH | | 219 | 4.603 | −22.061 | 0.460 | 1.00 | 53.96 | | O |
| ANISOU | 2939 | O | HOH | | 219 | 5514 | 7256 | 7732 | 2032 | −1336 | −251 | O |
| HETATM | 2940 | O | HOH | | 220 | 10.827 | −12.634 | 6.062 | 1.00 | 36.24 | | O |
| ANISOU | 2940 | O | HOH | | 220 | 4009 | 4964 | 4798 | 381 | 768 | 733 | O |
| HETATM | 2941 | O | HOH | | 221 | 3.604 | 31.446 | 23.751 | 1.00 | 46.85 | | O |
| ANISOU | 2941 | O | HOH | | 221 | 6678 | 5186 | 5936 | −2552 | 534 | 963 | O |
| HETATM | 2942 | O | HOH | | 222 | 0.515 | 13.682 | −0.599 | 1.00 | 68.62 | | O |
| ANISOU | 2942 | O | HOH | | 222 | 7850 | 9166 | 9056 | 510 | −452 | −1262 | O |
| HETATM | 2943 | O | HOH | | 223 | −12.650 | 4.013 | −10.449 | 1.00 | 49.70 | | O |
| ANISOU | 2943 | O | HOH | | 223 | 6515 | 5696 | 6672 | −1091 | −118 | 1170 | O |
| HETATM | 2944 | O | HOH | | 224 | 13.399 | −0.908 | −27.021 | 1.00 | 49.06 | | O |
| ANISOU | 2944 | O | HOH | | 224 | 3278 | 8270 | 7093 | 686 | 63 | −1803 | O |
| HETATM | 2945 | O | HOH | | 225 | −13.469 | −18.400 | −7.964 | 1.00 | 28.99 | | O |
| ANISOU | 2945 | O | HOH | | 225 | 3323 | 3864 | 3826 | −1193 | −477 | −421 | O |
| HETATM | 2946 | O | HOH | | 226 | 7.339 | −23.236 | −7.809 | 1.00 | 55.00 | | O |
| ANISOU | 2946 | O | HOH | | 226 | 6761 | 7437 | 6701 | 439 | 1061 | −1392 | O |
| HETATM | 2947 | O | HOH | | 227 | −14.169 | 7.887 | 14.283 | 1.00 | 35.79 | | O |
| ANISOU | 2947 | O | HOH | | 227 | 6533 | 3209 | 3856 | −515 | −2270 | −356 | O |
| HETATM | 2948 | O | HOH | | 228 | 13.185 | 2.612 | −11.315 | 1.00 | 26.65 | | O |
| ANISOU | 2948 | O | HOH | | 228 | 1973 | 3800 | 4353 | −497 | −224 | 508 | O |
| HETATM | 2949 | O | HOH | | 229 | 19.074 | −3.614 | 4.071 | 1.00 | 36.14 | | O |
| ANISOU | 2949 | O | HOH | | 229 | 2332 | 7516 | 3883 | 820 | 296 | −1523 | O |
| HETATM | 2950 | O | HOH | | 230 | −1.211 | 32.861 | 35.670 | 1.00 | 41.67 | | O |
| ANISOU | 2950 | O | HOH | | 230 | 5916 | 4627 | 5292 | 1535 | −2089 | −514 | O |
| HETATM | 2951 | O | HOH | | 231 | 2.312 | −21.900 | 1.259 | 1.00 | 48.91 | | O |
| ANISOU | 2951 | O | HOH | | 231 | 5363 | 4853 | 8366 | −2112 | 36 | 399 | O |
| HETATM | 2952 | O | HOH | | 232 | 21.643 | 11.750 | 20.878 | 1.00 | 30.90 | | O |
| ANISOU | 2952 | O | HOH | | 232 | 3909 | 3808 | 4024 | −666 | −444 | 364 | O |
| HETATM | 2953 | O | HOH | | 233 | 13.284 | 9.583 | 3.293 | 1.00 | 43.32 | | O |
| ANISOU | 2953 | O | HOH | | 233 | 5022 | 5685 | 5752 | −1708 | 2845 | −2175 | O |
| HETATM | 2954 | O | HOH | | 234 | −16.077 | −5.923 | −9.145 | 1.00 | 43.37 | | O |
| ANISOU | 2954 | O | HOH | | 234 | 6634 | 5589 | 4257 | −372 | 485 | 1025 | O |
| HETATM | 2955 | O | HOH | | 235 | −13.467 | −10.341 | −8.354 | 1.00 | 29.04 | | O |
| ANISOU | 2955 | O | HOH | | 235 | 2215 | 4825 | 3996 | 468 | 956 | 184 | O |
| HETATM | 2956 | O | HOH | | 236 | 16.524 | −13.999 | 5.910 | 1.00 | 35.62 | | O |
| ANISOU | 2956 | O | HOH | | 236 | 4436 | 6081 | 3019 | −458 | −750 | −391 | O |
| HETATM | 2957 | O | HOH | | 237 | −7.001 | 12.498 | 30.378 | 1.00 | 26.49 | | O |
| ANISOU | 2957 | O | HOH | | 237 | 2067 | 2853 | 5143 | −271 | 570 | −1097 | O |
| HETATM | 2958 | O | HOH | | 238 | −17.095 | −0.199 | 21.017 | 1.00 | 51.35 | | O |
| ANISOU | 2958 | O | HOH | | 238 | 6080 | 7469 | 5962 | −2016 | 1888 | −377 | O |
| HETATM | 2959 | O | HOH | | 239 | −10.801 | −12.077 | 6.659 | 1.00 | 41.99 | | O |
| ANISOU | 2959 | O | HOH | | 239 | 4334 | 6360 | 5260 | −1162 | 312 | 825 | O |
| HETATM | 2960 | O | HOH | | 240 | −8.489 | 9.228 | 3.543 | 1.00 | 34.82 | | O |
| ANISOU | 2960 | O | HOH | | 240 | 4488 | 3632 | 5109 | 730 | 387 | 738 | O |
| HETATM | 2961 | O | HOH | | 241 | −14.707 | 7.384 | 29.158 | 1.00 | 34.80 | | O |
| ANISOU | 2961 | O | HOH | | 241 | 5090 | 4770 | 3363 | −1164 | 921 | 354 | O |
| HETATM | 2962 | O | HOH | | 242 | −8.344 | 21.000 | 10.801 | 1.00 | 40.19 | | O |
| ANISOU | 2962 | O | HOH | | 242 | 3507 | 4562 | 7202 | −461 | −74 | −613 | O |
| HETATM | 2963 | O | HOH | | 243 | −13.128 | 24.123 | 14.260 | 1.00 | 42.82 | | O |
| ANISOU | 2963 | O | HOH | | 243 | 5998 | 5488 | 4785 | 1513 | −1338 | 1530 | O |
| HETATM | 2964 | O | HOH | | 244 | −17.476 | −14.871 | −2.757 | 1.00 | 37.57 | | O |
| ANISOU | 2964 | O | HOH | | 244 | 3325 | 6402 | 4547 | 709 | 406 | −784 | O |
| HETATM | 2965 | O | HOH | | 245 | 19.618 | 4.729 | 26.668 | 1.00 | 36.31 | | O |
| ANISOU | 2965 | O | HOH | | 245 | 3041 | 4682 | 6073 | 311 | 846 | −698 | O |
| HETATM | 2966 | O | HOH | | 246 | 13.980 | 8.850 | 29.941 | 1.00 | 56.14 | | O |
| ANISOU | 2966 | O | HOH | | 246 | 5369 | 8102 | 7860 | −443 | 1309 | −42 | O |
| HETATM | 2967 | O | HOH | | 247 | −8.987 | −19.895 | −1.244 | 1.00 | 42.89 | | O |
| ANISOU | 2967 | O | HOH | | 247 | 7116 | 5005 | 4177 | −42 | −639 | 2016 | O |
| HETATM | 2968 | O | HOH | | 248 | 1.566 | 9.016 | −6.652 | 1.00 | 38.46 | | O |
| ANISOU | 2968 | O | HOH | | 248 | 6938 | 2980 | 4696 | 846 | −1004 | 61 | O |
| HETATM | 2969 | O | HOH | | 249 | 13.662 | −16.427 | −14.268 | 1.00 | 54.90 | | O |
| ANISOU | 2969 | O | HOH | | 249 | 8241 | 6041 | 6576 | −1285 | 1548 | −630 | O |
| HETATM | 2970 | O | HOH | | 250 | −8.601 | −2.820 | 16.757 | 1.00 | 36.63 | | O |
| ANISOU | 2970 | O | HOH | | 250 | 5461 | 3027 | 5430 | 716 | 994 | 225 | O |
| HETATM | 2971 | O | HOH | | 251 | 10.577 | 0.348 | 31.893 | 1.00 | 49.06 | | O |
| ANISOU | 2971 | O | HOH | | 251 | 5979 | 6971 | 5688 | −399 | 154 | −318 | O |
| HETATM | 2972 | O | HOH | | 252 | −1.155 | 13.220 | 3.529 | 1.00 | 50.39 | | O |
| ANISOU | 2972 | O | HOH | | 252 | 8077 | 5707 | 5361 | 1425 | −1874 | 1 | O |
| HETATM | 2973 | O | HOH | | 253 | 11.331 | 0.873 | −2.001 | 1.00 | 64.09 | | O |
| ANISOU | 2973 | O | HOH | | 253 | 7806 | 9116 | 7430 | 686 | 379 | −640 | O |
| HETATM | 2974 | O | HOH | | 254 | 7.598 | −8.019 | −29.795 | 1.00 | 34.92 | | O |
| ANISOU | 2974 | O | HOH | | 254 | 3970 | 5816 | 3482 | −616 | 1662 | −1245 | O |
| HETATM | 2975 | O | HOH | | 255 | −11.022 | 6.528 | 0.345 | 1.00 | 40.11 | | O |
| ANISOU | 2975 | O | HOH | | 255 | 3363 | 6641 | 5235 | 1608 | −565 | −1531 | O |
| HETATM | 2976 | O | HOH | | 256 | 0.550 | 10.190 | 34.297 | 1.00 | 57.32 | | O |
| ANISOU | 2976 | O | HOH | | 256 | 7210 | 6055 | 8513 | 2146 | −1682 | 685 | O |
| HETATM | 2977 | O | HOH | | 257 | 8.109 | 7.355 | −8.064 | 1.00 | 37.35 | | O |
| ANISOU | 2977 | O | HOH | | 257 | 3766 | 5104 | 5322 | −2670 | 913 | −2333 | O |
| HETATM | 2978 | O | HOH | | 258 | −14.018 | 4.189 | 29.852 | 1.00 | 68.62 | | O |
| ANISOU | 2978 | O | HOH | | 258 | 8269 | 8737 | 9067 | −57 | 649 | 276 | O |
| HETATM | 2979 | O | HOH | | 259 | 3.404 | 10.704 | −19.008 | 1.00 | 36.02 | | O |
| ANISOU | 2979 | O | HOH | | 259 | 6108 | 3000 | 4577 | −1350 | −9 | 574 | O |
| HETATM | 2980 | O | HOH | | 260 | −17.262 | −16.815 | −3.963 | 1.00 | 39.44 | | O |
| ANISOU | 2980 | O | HOH | | 260 | 3536 | 5323 | 6127 | 195 | 81 | −410 | O |
| HETATM | 2981 | O | HOH | | 261 | 16.905 | −3.914 | 10.084 | 1.00 | 46.87 | | O |
| ANISOU | 2981 | O | HOH | | 261 | 5923 | 6007 | 5879 | 1413 | −1464 | 334 | O |
| HETATM | 2982 | O | HOH | | 262 | 5.063 | 12.664 | 32.963 | 1.00 | 45.71 | | O |
| ANISOU | 2982 | O | HOH | | 262 | 5310 | 5257 | 6801 | 2920 | −647 | −451 | O |
| HETATM | 2983 | O | HOH | | 263 | 1.933 | 1.493 | 30.076 | 1.00 | 37.19 | | O |
| ANISOU | 2983 | O | HOH | | 263 | 5110 | 4396 | 4625 | 166 | −596 | 589 | O |
| HETATM | 2984 | O | HOH | | 264 | 4.646 | −17.590 | 1.678 | 1.00 | 42.50 | | O |
| ANISOU | 2984 | O | HOH | | 264 | 7323 | 3081 | 5744 | 126 | −2175 | 2571 | O |
| HETATM | 2985 | O | HOH | | 265 | −17.717 | 19.821 | 19.766 | 1.00 | 30.42 | | O |
| ANISOU | 2985 | O | HOH | | 265 | 2559 | 5373 | 3625 | −25 | 1307 | −1204 | O |
| HETATM | 2986 | O | HOH | | 266 | 7.981 | −2.515 | −4.392 | 1.00 | 13.86 | | O |
| ANISOU | 2986 | O | HOH | | 266 | 1675 | 1561 | 2030 | −61 | −709 | 71 | O |
| HETATM | 2987 | O | HOH | | 267 | 5.336 | 25.713 | 24.978 | 1.00 | 23.36 | | O |
| ANISOU | 2987 | O | HOH | | 267 | 2361 | 4007 | 2508 | −95 | −211 | −437 | O |
| HETATM | 2988 | O | HOH | | 268 | −5.529 | 10.016 | 13.886 | 1.00 | 16.02 | | O |
| ANISOU | 2988 | O | HOH | | 268 | 1752 | 2117 | 2216 | −142 | −191 | −119 | O |
| HETATM | 2989 | O | HOH | | 269 | −2.818 | −7.452 | −22.959 | 1.00 | 21.40 | | O |
| ANISOU | 2989 | O | HOH | | 269 | 2066 | 3523 | 2542 | −274 | 0 | −497 | O |
| HETATM | 2990 | O | HOH | | 270 | −8.286 | 3.710 | 5.166 | 1.00 | 21.86 | | O |
| ANISOU | 2990 | O | HOH | | 270 | 3968 | 2056 | 2280 | −96 | 556 | −322 | O |
| HETATM | 2991 | O | HOH | | 271 | −8.231 | 8.629 | 29.192 | 1.00 | 26.12 | | O |
| ANISOU | 2991 | O | HOH | | 271 | 3606 | 2727 | 3593 | −883 | −1652 | 545 | O |
| HETATM | 2992 | O | HOH | | 272 | −9.280 | 6.038 | 28.935 | 1.00 | 20.74 | | O |
| ANISOU | 2992 | O | HOH | | 272 | 3006 | 2638 | 2235 | 213 | −723 | −101 | O |
| HETATM | 2993 | O | HOH | | 273 | 10.460 | −17.302 | −8.172 | 1.00 | 26.16 | | O |
| ANISOU | 2993 | O | HOH | | 273 | 3296 | 2652 | 3992 | −515 | −1434 | 541 | O |
| HETATM | 2994 | O | HOH | | 274 | 12.712 | 18.769 | 11.334 | 1.00 | 22.38 | | O |
| ANISOU | 2994 | O | HOH | | 274 | 2794 | 2955 | 2755 | 651 | −899 | −728 | O |
| HETATM | 2995 | O | HOH | | 275 | 5.543 | −2.292 | −28.884 | 1.00 | 24.82 | | O |
| ANISOU | 2995 | O | HOH | | 275 | 2103 | 3777 | 3551 | −27 | −227 | 1216 | O |
| HETATM | 2996 | O | HOH | | 276 | −8.873 | 4.577 | 8.781 | 1.00 | 25.16 | | O |
| ANISOU | 2996 | O | HOH | | 276 | 2925 | 3477 | 3158 | −501 | −617 | −771 | O |
| HETATM | 2997 | O | HOR | | 277 | 12.857 | −18.523 | −1.753 | 1.00 | 21.81 | | O |
| ANISOU | 2997 | O | HOH | | 277 | 3288 | 2666 | 2332 | 736 | −64 | −355 | O |
| HETATM | 2998 | O | HOH | | 278 | −6.986 | 22.048 | 12.425 | 1.00 | 25.72 | | O |
| ANISOU | 2998 | O | HOH | | 278 | 3384 | 3101 | 3288 | 658 | −1102 | 11 | O |
| HETATM | 2999 | O | HOH | | 279 | 7.379 | −4.477 | 11.926 | 1.00 | 23.87 | | O |
| ANISOU | 2999 | O | HOH | | 279 | 3564 | 2679 | 2825 | 181 | 235 | 506 | O |
| HETATM | 3000 | O | HOH | | 280 | 16.428 | 15.567 | 13.106 | 1.00 | 29.55 | | O |
| ANISOU | 3000 | O | HOH | | 280 | 1955 | 4311 | 4963 | 28 | −222 | −1919 | O |
| HETATM | 3001 | O | HOH | | 281 | 11.675 | −18.050 | −5.817 | 1.00 | 22.16 | | O |
| ANISOU | 3001 | O | HOH | | 281 | 3210 | 2427 | 2782 | −722 | 63 | −274 | O |
| HETATM | 3002 | O | HOH | | 282 | −4.856 | −5.830 | 9.661 | 1.00 | 28.02 | | O |
| ANISOU | 3002 | O | HOH | | 282 | 2584 | 4394 | 3670 | 1033 | 232 | −38 | O |
| HETATM | 3003 | O | HOH | | 283 | −2.136 | −12.075 | −21.417 | 1.00 | 31.75 | | O |
| ANISOU | 3003 | O | HOH | | 283 | 3602 | 5292 | 3171 | 1187 | −1490 | −1254 | O |
| HETATM | 3004 | O | HOH | | 284 | −13.485 | 10.927 | 19.179 | 1.00 | 28.40 | | O |
| ANISOU | 3004 | O | HOH | | 284 | 3528 | 3130 | 4135 | −538 | −649 | −788 | O |
| HETATM | 3005 | O | HOH | | 285 | −2.960 | 32.984 | 22.526 | 1.00 | 27.15 | | O |
| ANISOU | 3005 | O | HOH | | 285 | 3852 | 3572 | 2890 | 355 | −173 | 548 | O |
| HETATM | 3006 | O | HOH | | 286 | 16.054 | −7.196 | −6.921 | 1.00 | 31.59 | | O |
| ANISOU | 3006 | O | HOH | | 286 | 4694 | 3946 | 3364 | 1310 | −946 | −698 | O |
| HETATM | 3007 | O | HOH | | 287 | 15.906 | −13.505 | −15.047 | 1.00 | 31.95 | | O |
| ANISOU | 3007 | O | HOH | | 287 | 2001 | 4902 | 5238 | 526 | 118 | −572 | O |
| HETATM | 3008 | O | HOH | | 288 | 6.503 | −18.855 | 2.559 | 1.00 | 41.03 | | O |
| ANISOU | 3008 | O | HOH | | 288 | 5496 | 6566 | 3526 | −854 | −1919 | 2035 | O |
| HETATM | 3009 | O | HOH | | 289 | −14.785 | 26.337 | 17.507 | 1.00 | 31.93 | | O |
| ANISOU | 3009 | O | HOH | | 289 | 3322 | 4883 | 3927 | −31 | 4 | −215 | O |
| HETATM | 3010 | O | HOH | | 290 | −10.209 | 2.674 | −11.508 | 1.00 | 29.98 | | O |
| ANISOU | 3010 | O | HOH | | 290 | 4422 | 3896 | 3073 | −1567 | −963 | 54 | O |
| HETATM | 3011 | O | HOH | | 291 | −10.310 | 2.087 | 27.817 | 1.00 | 27.83 | | O |
| ANISOU | 3011 | O | HOH | | 291 | 4036 | 2939 | 3599 | −796 | −358 | 1218 | O |
| HETATM | 3012 | O | HOH | | 292 | −13.938 | 0.077 | −9.170 | 1.00 | 23.14 | | O |
| ANISOU | 3012 | O | HOH | | 292 | 2185 | 3830 | 2776 | 484 | −113 | −1072 | O |
| HETATM | 3013 | O | HOH | | 293 | 15.922 | 7.651 | 29.745 | 1.00 | 29.52 | | O |
| ANISOU | 3013 | O | HOH | | 293 | 3697 | 3218 | 4299 | 254 | −1582 | 84 | O |
| HETATM | 3014 | O | HOH | | 294 | −18.548 | 2.992 | 7.969 | 1.00 | 33.09 | | O |
| ANISOU | 3014 | O | HOH | | 294 | 3696 | 5386 | 3488 | −206 | 1137 | 167 | O |
| HETATM | 3015 | O | HOH | | 295 | −14.773 | 27.902 | 19.662 | 1.00 | 32.70 | | O |
| ANISOU | 3015 | O | HOH | | 295 | 2442 | 4679 | 5303 | 157 | 607 | −1627 | O |
| HETATM | 3016 | O | HOH | | 296 | 11.056 | −4.462 | 11.584 | 1.00 | 30.24 | | O |
| ANISOU | 3016 | O | HOH | | 296 | 4892 | 3459 | 3139 | −922 | −1295 | 169 | O |
| HETATM | 3017 | O | HOH | | 297 | −6.876 | −7.277 | 30.747 | 1.00 | 30.55 | | O |
| ANISOU | 3017 | O | HOH | | 297 | 2223 | 4699 | 4687 | 352 | −359 | −803 | O |
| HETATM | 3018 | O | HOR | | 298 | −5.039 | −8.906 | 6.727 | 1.00 | 36.83 | | O |
| ANISOU | 3018 | O | HOH | | 298 | 4829 | 3637 | 5526 | 297 | −233 | 1070 | O |
| HETATM | 3019 | O | HOH | | 299 | 14.746 | −3.124 | −6.082 | 1.00 | 27.52 | | O |
| ANISOU | 3019 | O | HOH | | 299 | 2213 | 5191 | 3051 | −642 | 30 | 655 | O |
| HETATM | 3020 | O | HOH | | 300 | 30.695 | −7.700 | 16.333 | 1.00 | 47.03 | | O |
| ANISOU | 3020 | O | HOH | | 300 | 6629 | 7059 | 4181 | −845 | 880 | −558 | O |
| HETATM | 3021 | O | HOH | | 301 | 0.155 | −15.241 | 2.730 | 1.00 | 28.31 | | O |
| ANISOU | 3021 | O | HOH | | 301 | 3105 | 3787 | 3867 | 1024 | −373 | 147 | O |
| HETATM | 3022 | O | HOH | | 302 | 10.631 | −16.441 | −19.989 | 1.00 | 34.45 | | O |
| ANISOU | 3022 | O | HOH | | 302 | 3313 | 4275 | 5502 | −687 | 1482 | −579 | O |
| HETATM | 3023 | O | HOH | | 303 | −0.167 | 16.659 | 3.885 | 1.00 | 42.68 | | O |
| ANISOU | 3023 | O | HOH | | 303 | 6393 | 6697 | 3126 | −2275 | 1353 | 3 | O |
| HETATM | 3024 | O | HOH | | 304 | 6.339 | 19.817 | 30.866 | 1.00 | 39.68 | | O |
| ANISOU | 3024 | O | HOH | | 304 | 3769 | 5842 | 5465 | −87 | −699 | −1555 | O |
| HETATM | 3025 | O | HOH | | 305 | 6.229 | 10.900 | −15.319 | 1.00 | 37.53 | | O |
| ANISOU | 3025 | O | HOH | | 305 | 5876 | 3723 | 4659 | −1691 | −1231 | 965 | O |
| HETATM | 3026 | O | HOH | | 306 | −0.730 | −5.402 | −30.337 | 1.00 | 32.38 | | O |
| ANISOU | 3026 | O | HOH | | 306 | 4057 | 4572 | 3676 | −491 | −1204 | 992 | O |
| HETATM | 3027 | O | HOH | | 307 | 2.353 | −22.650 | −15.690 | 1.00 | 33.53 | | O |
| ANISOU | 3027 | O | HOH | | 307 | 4978 | 3467 | 4296 | −215 | 172 | 1008 | O |
| HETATM | 3028 | O | HOH | | 308 | 7.370 | −2.270 | 15.814 | 1.00 | 35.12 | | O |
| ANISOU | 3028 | O | HOH | | 308 | 4175 | 4654 | 4516 | 1423 | 1379 | −961 | O |
| HETATM | 3029 | O | HOH | | 309 | 3.930 | −17.939 | −0.789 | 1.00 | 41.13 | | O |
| ANISOU | 3029 | O | HOH | | 309 | 5833 | 3031 | 6763 | 464 | −2502 | 1865 | O |
| HETATM | 3030 | O | HOH | | 310 | 19.084 | 17.545 | 17.041 | 1.00 | 60.42 | | O |
| ANISOU | 3030 | O | HOH | | 310 | 7478 | 9334 | 6143 | −89 | 1446 | −1412 | O |
| HETATM | 3031 | O | HOH | | 311 | −2.499 | 34.897 | 20.731 | 1.00 | 33.27 | | O |
| ANISOU | 3031 | O | HOH | | 311 | 5139 | 3072 | 4428 | 1075 | −510 | 660 | O |
| HETATM | 3032 | O | HOH | | 312 | 17.545 | −0.536 | −20.340 | 1.00 | 36.46 | | O |
| ANISOU | 3032 | O | HOH | | 312 | 3409 | 5612 | 4831 | −1232 | 1592 | −1969 | O |
| HETATM | 3033 | O | HOH | | 313 | 0.132 | 13.535 | 36.788 | 1.00 | 32.39 | | O |
| ANISOU | 3033 | O | HOH | | 313 | 3492 | 3983 | 4830 | 618 | 111 | 460 | O |
| HETATM | 3034 | O | HOH | | 314 | −3.167 | 10.063 | 34.071 | 1.00 | 52.03 | | O |
| ANISOU | 3034 | O | HOH | | 314 | 6779 | 6405 | 6585 | −1270 | −971 | −474 | O |
| HETATM | 3035 | O | HOH | | 315 | −5.835 | 6.711 | 32.183 | 1.00 | 50.58 | | O |
| ANISOU | 3035 | O | HOH | | 315 | 6776 | 6337 | 6106 | −1449 | 1145 | 920 | O |
| HETATM | 3036 | O | HOH | | 316 | −12.323 | 11.582 | 15.206 | 1.00 | 33.89 | | O |
| ANISOU | 3036 | O | HOH | | 316 | 2539 | 4318 | 6019 | 1439 | −916 | −757 | O |
| HETATM | 3037 | O | HOH | | 317 | 12.345 | −14.326 | −16.341 | 1.00 | 40.36 | | O |
| ANISOU | 3037 | O | HOH | | 317 | 2127 | 7355 | 5851 | 1343 | −999 | −60 | O |
| HETATM | 3038 | O | HOH | | 318 | 15.027 | 21.647 | 14.930 | 1.00 | 29.76 | | O |
| ANISOU | 3038 | O | HOH | | 318 | 3725 | 4182 | 3399 | −1344 | −1029 | 1265 | O |
| HETATM | 3039 | O | HOH | | 319 | 0.118 | −18.966 | 0.143 | 1.00 | 76.15 | | O |
| ANISOU | 3039 | O | HOH | | 319 | 10784 | 9705 | 8446 | −296 | −100 | 653 | O |
| HETATM | 3040 | O | HOH | | 320 | 6.084 | −20.620 | −9.538 | 1.00 | 44.66 | | O |
| ANISOU | 3040 | O | HOH | | 320 | 5924 | 4853 | 6192 | −436 | −693 | 262 | O |
| HETATM | 3041 | O | HOH | | 321 | 0.290 | −21.172 | 4.391 | 1.00 | 49.49 | | O |
| ANISOU | 3041 | O | HOH | | 321 | 6242 | 5793 | 6771 | 830 | −846 | 608 | O |
| HETATM | 3042 | O | HOH | | 322 | 0.328 | −6.488 | 9.128 | 1.00 | 47.57 | | O |
| ANISOU | 3042 | O | HOH | | 322 | 5704 | 6357 | 6012 | −358 | 563 | 372 | O |
| HETATM | 3043 | O | HOH | | 323 | −0.825 | 31.675 | 27.331 | 1.00 | 25.61 | | O |
| ANISOU | 3043 | O | HOH | | 323 | 3192 | 2206 | 4335 | −92 | −998 | −743 | O |
| HETATM | 3044 | O | HOH | | 324 | 1.400 | −20.762 | −11.980 | 1.00 | 47.31 | | O |
| ANISOU | 3044 | O | HOH | | 324 | 6253 | 5896 | 5825 | −1948 | 314 | 1843 | O |
| HETATM | 3045 | O | HOH | | 325 | 14.751 | −1.374 | −24.062 | 1.00 | 33.20 | | O |
| ANISOU | 3045 | O | HOH | | 325 | 3067 | 6213 | 3335 | −560 | 836 | 1348 | O |
| HETATM | 3046 | O | HOH | | 326 | 11.928 | 28.091 | 14.458 | 1.00 | 50.63 | | O |
| ANISOU | 3046 | O | HOH | | 326 | 6286 | 6706 | 6245 | 545 | 992 | 357 | O |
| HETATM | 3047 | O | HOH | | 327 | 0.287 | 1.316 | −25.503 | 1.00 | 35.02 | | O |
| ANISOU | 3047 | O | HOH | | 327 | 5233 | 5158 | 2916 | −115 | −541 | 991 | O |
| HETATM | 3048 | O | HOH | | 328 | 7.457 | 27.297 | 19.894 | 1.00 | 37.86 | | O |
| ANISOU | 3048 | O | HOH | | 328 | 4646 | 5724 | 4015 | −463 | 1562 | −105 | O |
| HETATM | 3049 | O | HOH | | 329 | 0.443 | −0.294 | 29.702 | 1.00 | 35.93 | | O |
| ANISOU | 3049 | O | HOH | | 329 | 3742 | 4576 | 5334 | −284 | −1732 | −531 | O |
| HETATM | 3050 | O | HOH | | 330 | 12.673 | −5.610 | −21.820 | 1.00 | 42.98 | | O |
| ANISOU | 3050 | O | HOH | | 330 | 5765 | 5967 | 4597 | 1007 | −829 | −1679 | O |
| HETATM | 3051 | O | HOH | | 331 | −10.922 | 0.273 | −21.618 | 1.00 | 33.04 | | O |
| ANISOU | 3051 | O | HOH | | 331 | 3837 | 4756 | 3961 | −49 | −263 | 317 | O |
| HETATM | 3052 | O | HOH | | 332 | −17.656 | −6.798 | −11.401 | 1.00 | 32.60 | | O |
| ANISOU | 3052 | O | HOH | | 332 | 2642 | 5366 | 4378 | −732 | 1038 | −446 | O |
| HETATM | 3053 | O | HOH | | 333 | −2.922 | −18.902 | −1.821 | 1.00 | 47.83 | | O |
| ANISOU | 3053 | O | HOH | | 333 | 7191 | 3692 | 7293 | −391 | −173 | 967 | O |
| HETATM | 3054 | O | HOH | | 334 | 14.858 | 11.493 | 4.013 | 1.00 | 42.96 | | O |
| ANISOU | 3054 | O | HOH | | 334 | 4665 | 6768 | 4892 | 2120 | −95 | −2036 | O |
| HETATM | 3055 | O | HOH | | 335 | −18.070 | −11.981 | 2.673 | 1.00 | 37.42 | | O |
| ANISOU | 3055 | O | HOH | | 335 | 5675 | 4119 | 4423 | −593 | −2006 | 1438 | O |
| HETATM | 3056 | O | HOH | | 336 | 16.967 | −2.985 | −9.547 | 1.00 | 36.86 | | O |
| ANISOU | 3056 | O | HOH | | 336 | 2788 | 6523 | 4695 | −817 | −635 | −536 | O |
| HETATM | 3057 | O | HOH | | 337 | 13.559 | −2.914 | 19.204 | 1.00 | 45.19 | | O |
| ANISOU | 3057 | O | HOH | | 337 | 7523 | 3813 | 5834 | 238 | −2095 | 2061 | O |
| HETATM | 3058 | O | HOH | | 338 | 20.220 | −4.404 | −15.662 | 1.00 | 27.55 | | O |
| ANISOU | 3058 | O | HOH | | 338 | 2833 | 3951 | 3683 | 551 | 560 | 270 | O |
| HETATM | 3059 | O | HOH | | 339 | 2.246 | 17.421 | −1.451 | 1.00 | 39.52 | | O |
| ANISOU | 3059 | O | HOH | | 339 | 4271 | 5145 | 5601 | 96 | 1473 | −1383 | O |
| HETATM | 3060 | O | HOH | | 340 | 9.297 | 24.617 | 2.842 | 1.00 | 45.82 | | O |
| ANISOU | 3060 | O | HOH | | 340 | 5706 | 7447 | 4256 | −1977 | 2443 | −1246 | O |
| HETATM | 3061 | O | HOH | | 341 | −5.238 | 24.002 | 35.705 | 1.00 | 31.96 | | O |
| ANISOU | 3061 | O | HOH | | 341 | 3505 | 4659 | 3978 | −214 | 1644 | −1186 | O |
| HETATM | 3062 | O | HOH | | 342 | −9.182 | −22.534 | −2.411 | 1.00 | 49.92 | | O |
| ANISOU | 3062 | O | HOH | | 342 | 6944 | 6871 | 5155 | −2355 | 1145 | −120 | O |
| HETATM | 3063 | O | HOH | | 343 | −3.732 | 13.781 | −16.653 | 1.00 | 38.43 | | O |
| ANISOU | 3063 | O | HOH | | 343 | 5548 | 3942 | 5112 | 1113 | 47 | 572 | O |
| HETATM | 3064 | O | HOH | | 344 | 9.138 | 4.795 | −2.367 | 1.00 | 49.54 | | O |
| ANISOU | 3064 | O | HOH | | 344 | 7188 | 5500 | 6136 | −1590 | −887 | 753 | O |
| HETATM | 3065 | O | HOH | | 345 | 19.818 | 14.775 | 20.361 | 1.00 | 37.08 | | O |
| ANISOU | 3065 | O | HOH | | 345 | 2799 | 5404 | 5888 | 2358 | −408 | −1666 | O |
| HETATM | 3066 | O | HOH | | 346 | 4.789 | 33.229 | 11.275 | 1.00 | 36.27 | | O |
| ANISOU | 3066 | O | HOH | | 346 | 4926 | 4622 | 4235 | −1986 | 1092 | −83 | O |
| HETATM | 3067 | O | HOH | | 347 | 3.364 | 3.480 | −0.308 | 1.00 | 65.05 | | O |
| ANISOU | 3067 | O | HOH | | 347 | 9774 | 6778 | 8164 | 708 | −1073 | 451 | O |
| HETATM | 3068 | O | HOH | | 348 | 31.059 | −7.050 | 13.935 | 1.00 | 44.37 | | O |
| ANISOU | 3068 | O | HOH | | 348 | 5755 | 5378 | 5728 | −459 | −223 | 1096 | O |
| HETATM | 3069 | O | HOH | | 349 | 1.844 | 2.784 | −26.535 | 1.00 | 42.81 | | O |
| ANISOU | 3069 | O | HOH | | 349 | 3680 | 5562 | 7025 | 1060 | −169 | −934 | O |
| HETATM | 3070 | O | HOH | | 350 | −2.791 | 8.203 | −0.537 | 1.00 | 47.88 | | O |
| ANISOU | 3070 | O | HOH | | 350 | 4360 | 7610 | 6224 | 298 | 1620 | −975 | O |
| HETATM | 3071 | O | HOH | | 351 | 4.513 | 22.359 | 29.078 | 1.00 | 35.35 | | O |
| ANISOU | 3071 | O | HOH | | 351 | 3956 | 5328 | 4147 | −560 | 524 | −2308 | O |
| HETATM | 3072 | O | HOH | | 352 | −10.748 | −17.194 | 0.212 | 1.00 | 58.37 | | O |
| ANISOU | 3072 | O | HOH | | 352 | 9720 | 7089 | 5371 | −1802 | −183 | 2952 | O |
| HETATM | 3073 | O | HOH | | 353 | 1.206 | 4.628 | −1.928 | 1.00 | 42.03 | | O |
| ANISOU | 3073 | O | HOH | | 353 | 7294 | 3907 | 4768 | −417 | −715 | 1236 | O |
| HETATM | 3074 | O | HOH | | 354 | 16.847 | −8.026 | 5.913 | 1.00 | 32.17 | | O |
| ANISOU | 3074 | O | HOH | | 354 | 3793 | 5026 | 3403 | 515 | −731 | −1090 | O |
| HETATM | 3075 | O | HOH | | 355 | −8.324 | −5.416 | 9.955 | 1.00 | 40.44 | | O |
| ANISOU | 3075 | O | HOH | | 355 | 5539 | 4971 | 4856 | 834 | 87 | −1466 | O |
| HETATM | 3076 | O | HOH | | 356 | −11.571 | 9.208 | 15.902 | 1.00 | 41.42 | | O |
| ANISOU | 3076 | O | HOH | | 356 | 3714 | 5764 | 6261 | 294 | 666 | 665 | O |
| HETATM | 3077 | O | HOH | | 357 | −19.160 | −13.986 | 2.555 | 1.00 | 42.80 | | O |
| ANISOU | 3077 | O | HOH | | 357 | 7005 | 6185 | 3071 | −1084 | −382 | 1463 | O |
| HETATM | 3078 | O | HOH | | 358 | −12.572 | 7.102 | −2.228 | 1.00 | 41.25 | | O |
| ANISOU | 3078 | O | HOH | | 358 | 4487 | 5901 | 5286 | −985 | 1456 | −1352 | O |
| HETATM | 3079 | O | HOH | | 359 | 9.488 | −10.805 | 5.785 | 1.00 | 45.32 | | O |
| ANISOU | 3079 | O | HOH | | 359 | 5987 | 5325 | 5906 | 1446 | 439 | −311 | O |
| HETATM | 3080 | O | HOH | | 360 | 17.167 | 16.958 | 3.750 | 1.00 | 47.29 | | O |
| ANISOU | 3080 | O | HOH | | 360 | 3314 | 8424 | 6229 | 561 | 2032 | −1539 | O |
| HETATM | 3081 | O | HOH | | 361 | 6.524 | −2.315 | 18.886 | 1.00 | 45.69 | | O |
| ANISOU | 3081 | O | HOH | | 361 | 4688 | 5723 | 6948 | 1701 | 122 | −659 | O |
| HETATM | 3082 | O | HOH | | 362 | −0.123 | −5.460 | 13.639 | 1.00 | 59.72 | | O |
| ANISOU | 3082 | O | HOH | | 362 | 7209 | 8001 | 7481 | 891 | −92 | −140 | O |
| HETATM | 3083 | O | HOH | | 363 | −1.786 | 14.140 | 1.159 | 1.00 | 48.03 | | O |
| ANISOU | 3083 | O | HOH | | 363 | 5094 | 6473 | 6681 | −659 | 230 | −316 | O |
| HETATM | 3084 | O | HOH | | 364 | −2.248 | 10.723 | −3.268 | 1.00 | 50.44 | | O |
| ANISOU | 3084 | O | HOH | | 364 | 6608 | 5179 | 7379 | 215 | −713 | −2184 | O |
| HETATM | 3085 | O | HOH | | 365 | 0.789 | −8.737 | −33.525 | 1.00 | 50.21 | | O |
| ANISOU | 3085 | O | HOH | | 365 | 6518 | 5370 | 7189 | −3157 | 426 | 613 | O |
| HETATM | 3086 | O | HOH | | 366 | −13.523 | 10.959 | −9.483 | 1.00 | 53.97 | | O |
| ANISOU | 3086 | O | HOH | | 366 | 6635 | 6820 | 7052 | 1072 | −1573 | 194 | O |
| HETATM | 3087 | O | HOH | | 367 | −6.604 | 28.809 | 10.423 | 1.00 | 51.63 | | O |
| ANISOU | 3087 | O | HOH | | 367 | 7022 | 7008 | 5589 | 1702 | −3667 | 928 | O |
| HETATM | 3088 | O | HOH | | 368 | 9.351 | −17.188 | −12.471 | 1.00 | 33.64 | | O |
| ANISOU | 3088 | O | HOH | | 368 | 1692 | 7865 | 3223 | 547 | −657 | 316 | O |
| HETATM | 3089 | O | HOH | | 369 | 1.675 | −1.037 | 22.490 | 1.00 | 46.76 | | O |
| ANISOU | 3089 | O | HOH | | 369 | 7274 | 5018 | 5475 | −2081 | 915 | 1318 | O |
| HETATM | 3090 | O | HOH | | 370 | −13.676 | 30.870 | 18.494 | 1.00 | 44.37 | | O |
| ANISOU | 3090 | O | HOH | | 370 | 5357 | 6553 | 4948 | −395 | 810 | 526 | O |
| HETATM | 3091 | O | HOH | | 371 | −7.126 | 22.441 | 31.472 | 1.00 | 44.14 | | O |
| ANISOU | 3091 | O | HOH | | 371 | 2564 | 7151 | 7057 | 761 | 83 | −192 | O |
| HETATM | 3092 | O | HOH | | 372 | 18.660 | 13.367 | 18.963 | 1.00 | 35.38 | | O |
| ANISOU | 3092 | O | HOH | | 372 | 4092 | 4358 | 4991 | 950 | 62 | −1016 | O |
| HETATM | 3093 | O | HOH | | 373 | −5.294 | 10.450 | −20.141 | 1.00 | 64.06 | | O |
| ANISOU | 3093 | O | HOH | | 373 | 9820 | 6866 | 7655 | 569 | −3305 | 2169 | O |
| HETATM | 3094 | O | HOH | | 374 | −5.947 | 9.639 | −5.338 | 1.00 | 59.16 | | O |
| ANISOU | 3094 | O | HOH | | 374 | 5022 | 8657 | 8801 | −2345 | 1225 | 47 | O |
| HETATM | 3095 | O | HOH | | 375 | −1.150 | 31.258 | 33.864 | 1.00 | 50.21 | | O |
| ANISOU | 3095 | O | HOH | | 375 | 4837 | 8484 | 5755 | 98 | 619 | −2159 | O |
| HETATM | 3096 | O | HOH | | 376 | −8.834 | 33.731 | 17.108 | 1.00 | 48.68 | | O |
| ANISOU | 3096 | O | HOH | | 376 | 6574 | 4595 | 7326 | −110 | −710 | 1517 | O |
| HETATM | 3097 | O | HOH | | 377 | −14.693 | 25.287 | 15.474 | 1.00 | 63.36 | | O |
| ANISOU | 3097 | O | HOH | | 377 | 8077 | 8143 | 7854 | 857 | 1189 | −244 | O |
| HETATM | 3098 | O | HOH | | 378 | −12.635 | −13.658 | 7.088 | 1.00 | 54.89 | | O |
| ANISOU | 3098 | O | HOH | | 378 | 7929 | 6135 | 6791 | −771 | −289 | 501 | O |
| HETATM | 3099 | O | HOH | | 379 | 7.216 | 29.176 | 8.106 | 1.00 | 60.20 | | O |
| ANISOU | 3099 | O | HOH | | 379 | 7724 | 7535 | 7616 | −955 | −304 | −567 | O |
| HETATM | 3100 | O | HOH | | 380 | −1.367 | −15.597 | −21.887 | 1.00 | 44.16 | | O |
| ANISOU | 3100 | O | HOH | | 380 | 5823 | 6187 | 4767 | −358 | −740 | −42 | O |
| HETATM | 3101 | O | HOH | | 381 | −3.570 | −4.830 | −26.678 | 1.00 | 44.66 | | O |
| ANISOU | 3101 | O | HOH | | 381 | 5681 | 6241 | 5048 | 1066 | −1786 | −599 | O |
| HETATM | 3102 | O | HOH | | 382 | 19.193 | −4.694 | −12.487 | 1.00 | 41.74 | | O |
| ANISOU | 3102 | O | HOH | | 382 | 4471 | 6494 | 4893 | −867 | −422 | −1163 | O |
| HETATM | 3103 | O | HOH | | 383 | −8.215 | 12.566 | −12.290 | 1.00 | 41.68 | | O |
| ANISOU | 3103 | O | HOH | | 383 | 4172 | 5125 | 6538 | 2185 | 361 | 195 | O |
| HETATM | 3104 | O | HOH | | 384 | −16.593 | 30.451 | 19.731 | 1.00 | 32.58 | | O |
| ANISOU | 3104 | O | HOH | | 384 | 5757 | 4257 | 2367 | −1355 | −343 | 857 | O |
| HETATM | 3105 | O | HOH | | 385 | 30.515 | −9.985 | 17.929 | 1.00 | 25.46 | | O |
| ANISOU | 3105 | O | HOH | | 385 | 3539 | 3435 | 2701 | 1057 | 389 | −100 | O |
| HETATM | 3106 | O | HOH | | 386 | −7.785 | 5.007 | 30.901 | 1.00 | 27.24 | | O |
| ANISOU | 3106 | O | HOH | | 386 | 3858 | 4491 | 2003 | 1061 | −227 | −38 | O |
| HETATM | 3107 | O | HOH | | 387 | 10.599 | −20.423 | −5.255 | 1.00 | 33.13 | | O |
| ANISOU | 3107 | O | HOH | | 387 | 6277 | 2704 | 3608 | −652 | 287 | 117 | O |
| HETATM | 3108 | O | HOH | | 388 | 5.341 | −6.298 | 11.755 | 1.00 | 31.03 | | O |
| ANISOU | 3108 | O | HOH | | 388 | 3859 | 4163 | 3767 | −439 | 212 | 80 | O |
| HETATM | 3109 | O | HOH | | 389 | −5.799 | 32.003 | 21.991 | 1.00 | 27.97 | | O |
| ANISOU | 3109 | O | HOH | | 389 | 3825 | 3695 | 3107 | 853 | 226 | 459 | O |
| HETATM | 3110 | O | HOH | | 390 | 17.234 | −1.654 | −23.331 | 1.00 | 34.84 | | O |
| ANISOU | 3110 | O | HOH | | 390 | 2085 | 5971 | 5182 | −178 | 1183 | −1100 | O |
| HETATM | 3111 | O | HOH | | 391 | 18.467 | 2.232 | 23.587 | 1.00 | 53.35 | | O |
| ANISOU | 3111 | O | HOH | | 391 | 6179 | 7045 | 7046 | −1955 | 2851 | −846 | O |
| HETATM | 3112 | O | HOH | | 392 | 29.545 | −9.331 | 11.188 | 1.00 | 44.65 | | O |
| ANISOU | 3112 | O | HOH | | 392 | 5235 | 6572 | 5158 | 1538 | 747 | −1846 | O |
| HETATM | 3113 | O | HOH | | 393 | −2.873 | −6.354 | 11.737 | 1.00 | 36.36 | | O |
| ANISOU | 3113 | O | HOH | | 393 | 6311 | 3418 | 4087 | 836 | −96 | −31 | O |
| HETATM | 3114 | O | HOH | | 394 | −16.180 | −14.472 | −0.421 | 1.00 | 45.02 | | O |
| ANISOU | 3114 | O | HOH | | 394 | 5230 | 5934 | 5941 | 2418 | −1096 | −417 | O |
| HETATM | 3115 | O | HOH | | 395 | 18.400 | 11.854 | 20.674 | 1.00 | 33.67 | | O |
| ANISOU | 3115 | O | HOH | | 395 | 5286 | 3750 | 3758 | 1935 | 218 | −442 | O |
| HETATM | 3116 | O | HOH | | 396 | 13.025 | 0.236 | 1.070 | 1.00 | 43.38 | | O |
| ANISOU | 3116 | O | HOH | | 396 | 8384 | 5033 | 3066 | 522 | −280 | −677 | O |
| HETATM | 3117 | O | HOH | | 397 | −9.118 | −14.291 | 2.196 | 1.00 | 50.74 | | O |
| ANISOU | 3117 | O | HOH | | 397 | 8563 | 6495 | 4222 | −1270 | 626 | 1741 | O |
| HETATM | 3118 | O | HOH | | 398 | 9.023 | 19.781 | −1.378 | 1.00 | 53.14 | | O |
| ANISOU | 3118 | O | HOH | | 398 | 4590 | 8565 | 7038 | 139 | 1252 | −618 | O |
| HETATM | 3119 | O | HOH | | 399 | −8.145 | 16.579 | 34.749 | 1.00 | 46.43 | | O |
| ANISOU | 3119 | O | HOH | | 399 | 4934 | 7985 | 4720 | −1622 | 2855 | −856 | O |
| HETATM | 3120 | O | HOH | | 400 | −13.420 | 28.278 | 14.333 | 1.00 | 40.29 | | O |
| ANISOU | 3120 | O | HOH | | 400 | 6009 | 3784 | 5517 | 70 | −2554 | 835 | O |
| HETATM | 3121 | O | HOH | | 401 | −11.864 | 8.269 | 10.823 | 1.00 | 42.29 | | O |
| ANISOU | 3121 | O | HOH | | 401 | 5025 | 6314 | 4731 | −59 | −1226 | −792 | O |
| HETATM | 3122 | O | HOH | | 402 | 15.279 | 6.508 | 31.634 | 1.00 | 62.81 | | O |
| ANISOU | 3122 | O | HOH | | 402 | 8316 | 7738 | 7810 | 107 | −1320 | 128 | O |
| HETATM | 3123 | O | HOH | | 403 | 7.910 | 26.981 | 22.122 | 1.00 | 39.66 | | O |
| ANISOU | 3123 | O | HOH | | 403 | 4058 | 6323 | 4688 | −749 | 677 | −1152 | O |
| HETATM | 3124 | O | HOH | | 404 | 19.299 | −3.982 | −10.315 | 1.00 | 56.28 | | O |
| ANISOU | 3124 | O | HOH | | 404 | 6271 | 7804 | 7308 | −1363 | 1092 | −154 | O |
| HETATM | 3125 | O | HOH | | 405 | −3.340 | −6.709 | −25.242 | 1.00 | 39.74 | | O |
| ANISOU | 3125 | O | HOH | | 405 | 5853 | 4612 | 4635 | −1130 | −684 | −2051 | O |
| HETATM | 3126 | O | HOH | | 406 | −10.231 | 11.297 | −0.254 | 1.00 | 49.44 | | O |
| ANISOU | 3126 | O | HOH | | 406 | 5226 | 6565 | 6995 | 1696 | −1410 | 139 | O |
| HETATM | 3127 | O | HOH | | 407 | 2.342 | 11.700 | −8.912 | 1.00 | 45.67 | | O |
| ANISOU | 3127 | O | HOH | | 407 | 4737 | 6224 | 6390 | 378 | 2103 | −998 | O |
| HETATM | 3128 | O | HOH | | 408 | −0.153 | −17.720 | 2.787 | 1.00 | 41.38 | | O |
| ANISOU | 3128 | O | HOH | | 408 | 5449 | 4946 | 5326 | −147 | −188 | 35 | O |
| HETATM | 3129 | O | HOH | | 409 | −4.730 | −3.412 | −28.549 | 1.00 | 51.08 | | O |
| ANISOU | 3129 | O | HOH | | 409 | 7969 | 5323 | 6115 | −735 | −732 | −260 | O |
| HETATM | 3130 | O | HOH | | 410 | −16.968 | 9.501 | 14.197 | 1.00 | 30.50 | | O |
| ANISOU | 3130 | O | HOH | | 410 | 2664 | 4402 | 4524 | 221 | −326 | 485 | O |
| HETATM | 3131 | O | HOH | | 411 | −17.650 | −3.635 | 22.378 | 1.00 | 36.30 | | O |
| ANISOU | 3131 | O | HOH | | 411 | 4043 | 5016 | 4733 | 999 | 304 | 596 | O |
| HETATM | 3132 | O | HOH | | 412 | 5.455 | −4.979 | 19.371 | 1.00 | 55.21 | | O |
| ANISOU | 3132 | O | HOH | | 412 | 7675 | 6376 | 6926 | −870 | 452 | 127 | O |
| HETATM | 3133 | O | HOH | | 413 | 19.460 | 5.454 | 23.891 | 1.00 | 52.06 | | O |
| ANISOU | 3133 | O | HOH | | 413 | 5291 | 7913 | 6577 | −630 | 2395 | −2523 | O |
| HETATM | 3134 | O | HOH | | 414 | −7.317 | 8.727 | 31.937 | 1.00 | 43.29 | | O |
| ANISOU | 3134 | O | HOH | | 414 | 5664 | 5436 | 5347 | −497 | −1726 | −115 | O |
| HETATM | 3135 | O | HOH | | 415 | −12.831 | 33.308 | 15.929 | 1.00 | 66.28 | | O |
| ANISOU | 3135 | O | HOH | | 415 | 6426 | 8555 | 10201 | 3290 | −784 | −916 | O |
| HETATM | 3136 | O | HOH | | 416 | 2.415 | 11.855 | 3.404 | 1.00 | 45.89 | | O |
| ANISOU | 3136 | O | HOH | | 416 | 6950 | 4784 | 5701 | −2541 | 443 | 723 | O |
| HETATM | 3137 | O | HOH | | 417 | 15.945 | −5.999 | 10.047 | 1.00 | 49.83 | | O |
| ANISOU | 3137 | O | HOH | | 417 | 8066 | 5308 | 5557 | −1158 | −2343 | 874 | O |
| HETATM | 3138 | O | HOH | | 418 | 4.369 | 10.635 | 30.789 | 1.00 | 36.41 | | O |
| ANISOU | 3138 | O | HOH | | 418 | 3531 | 6616 | 3688 | 589 | 741 | 403 | O |
| HETATM | 3139 | O | HOH | | 419 | −4.587 | 12.224 | 34.534 | 1.00 | 50.62 | | O |
| ANISOU | 3139 | O | HOH | | 419 | 6111 | 6665 | 6457 | −892 | 926 | 204 | O |
| HETATM | 3140 | O | HOH | | 420 | 16.003 | 1.464 | −14.437 | 1.00 | 44.14 | | O |
| ANISOU | 3140 | O | HOH | | 420 | 4267 | 5266 | 7238 | −1836 | −19 | −2672 | O |
| HETATM | 3141 | O | HOH | | 421 | −17.340 | −5.032 | 3.292 | 1.00 | 50.23 | | O |
| ANISOU | 3141 | O | HOH | | 421 | 6011 | 7300 | 5773 | −1781 | 701 | 184 | O |
| HETATM | 3142 | O | HOH | | 422 | −11.365 | 6.798 | 30.340 | 1.00 | 48.76 | | O |
| ANISOU | 3142 | O | HOH | | 422 | 6581 | 6538 | 5406 | 51 | 910 | 752 | O |
| HETATM | 3143 | O | HOH | | 423 | 2.379 | 11.804 | −15.558 | 1.00 | 39.12 | | O |
| ANISOU | 3143 | O | HOH | | 423 | 5223 | 3701 | 5938 | 26 | 647 | 779 | O |
| HETATM | 3144 | O | HOH | | 424 | −19.196 | −3.380 | −3.793 | 1.00 | 31.28 | | O |
| ANISOU | 3144 | O | HOH | | 424 | 3731 | 4189 | 3966 | 1241 | 300 | −693 | O |
| HETATM | 3145 | O | HOH | | 425 | −0.361 | 34.754 | 18.427 | 1.00 | 47.31 | | O |
| ANISOU | 3145 | O | HOH | | 425 | 6353 | 5162 | 6462 | −2698 | 30 | 1652 | O |
| HETATM | 3146 | O | HOH | | 426 | 14.944 | −18.083 | −12.099 | 1.00 | 50.06 | | O |
| ANISOU | 3146 | O | HOH | | 426 | 5667 | 6846 | 6507 | −875 | 909 | −1657 | O |
| HETATM | 3147 | O | HOH | | 427 | −4.968 | 3.935 | −22.481 | 1.00 | 38.00 | | O |
| ANISOU | 3147 | O | HOH | | 427 | 4270 | 4618 | 5550 | −67 | 140 | −550 | O |
| HETATM | 3148 | O | HOH | | 428 | −13.223 | 4.282 | −6.220 | 1.00 | 40.38 | | O |
| ANISOU | 3148 | O | HOH | | 428 | 3648 | 4952 | 6742 | 1005 | −61 | −565 | O |
| HETATM | 3149 | O | HOH | | 429 | −15.214 | 10.554 | −5.282 | 1.00 | 95.31 | | O |
| ANISOU | 3149 | O | HOH | | 429 | 13222 | 10938 | 12054 | −424 | −664 | 270 | O |
| HETATM | 3150 | O | HOH | | 430 | 7.708 | 6.181 | −5.415 | 1.00 | 45.59 | | O |
| ANISOU | 3150 | O | HOH | | 430 | 4679 | 6400 | 6244 | −928 | −1871 | −1569 | O |
| HETATM | 3151 | O | HOH | | 431 | 3.068 | −1.696 | 24.940 | 1.00 | 59.04 | | O |
| ANISOU | 3151 | O | HOH | | 431 | 7480 | 7540 | 7411 | 544 | −133 | −738 | O |
| HETATM | 3152 | O | HOH | | 432 | 21.423 | −5.130 | −12.829 | 1.00 | 50.90 | | O |
| ANISOU | 3152 | O | HOH | | 432 | 7108 | 6254 | 5978 | −485 | −635 | −588 | O |
| HETATM | 3153 | O | HOH | | 433 | 23.833 | −4.409 | 10.392 | 1.00 | 57.64 | | O |
| ANISOU | 3153 | O | HOH | | 433 | 9408 | 6479 | 6014 | −734 | −1477 | 2660 | O |
| HETATM | 3154 | O | HOH | | 434 | 17.853 | 16.734 | 15.277 | 1.00 | 55.00 | | O |
| ANISOU | 3154 | O | HOH | | 434 | 6325 | 6836 | 7736 | 1178 | −11 | 88 | O |
| HETATM | 3155 | O | HOH | | 435 | −14.450 | −2.829 | 4.998 | 1.00 | 83.29 | | O |
| ANISOU | 3155 | O | HOH | | 435 | 9970 | 10758 | 10920 | 60 | 355 | −447 | O |
| HETATM | 3156 | O | HOH | | 436 | 19.154 | 17.051 | 19.347 | 1.00 | 46.49 | | O |
| ANISOU | 3156 | O | HOH | | 436 | 6656 | 6155 | 4854 | 143 | −1403 | 529 | O |
| HETATM | 3157 | O | HOH | | 437 | −9.142 | 17.529 | 7.268 | 1.00 | 39.70 | | O |
| ANISOU | 3157 | O | HOH | | 437 | 5187 | 5659 | 4237 | −777 | −1633 | −60 | O |
| HETATM | 3158 | O | HOH | | 438 | −5.754 | 11.777 | −16.318 | 1.00 | 60.09 | | O |
| ANISOU | 3158 | O | HOH | | 438 | 7542 | 7086 | 8203 | −1513 | 1535 | 495 | O |
| HETATM | 3159 | O | HOH | | 439 | 15.894 | 5.682 | 1.252 | 1.00 | 37.98 | | O |
| ANISOU | 3159 | O | HOH | | 439 | 4915 | 5156 | 4359 | −947 | 296 | 232 | O |
| HETATM | 3160 | O | HOH | | 440 | 4.551 | −4.007 | 22.986 | 1.00 | 55.32 | | O |
| ANISOU | 3160 | O | HOH | | 440 | 7145 | 6941 | 6932 | −765 | −1068 | −460 | O |
| HETATM | 3161 | O | HOH | | 441 | 13.117 | −1.846 | −4.384 | 1.00 | 76.87 | | O |
| ANISOU | 3161 | O | HOH | | 441 | 9521 | 10408 | 9279 | −1004 | 808 | −465 | O |
| HETATM | 3162 | O | HOH | | 442 | 12.297 | −14.822 | −20.418 | 1.00 | 45.29 | | O |
| ANISOU | 3162 | O | HOH | | 442 | 4958 | 5959 | 6290 | 1023 | 1692 | 21 | O |
| HETATM | 3163 | O | HOH | | 443 | 0.495 | −20.372 | −9.657 | 1.00 | 48.85 | | O |
| ANISOU | 3163 | O | HOH | | 443 | 7419 | 6359 | 4781 | −2610 | −590 | 1135 | O |
| HETATM | 3164 | O | HOH | | 444 | 1.873 | −19.020 | 2.877 | 1.00 | 51.34 | | O |
| ANISOU | 3164 | O | HOH | | 444 | 3214 | 8784 | 7509 | −8 | 492 | −586 | O |
| HETATM | 3165 | O | HOH | | 445 | −8.128 | 20.966 | 4.924 | 1.00 | 75.56 | | O |
| ANISOU | 3165 | O | HOH | | 445 | 9109 | 10203 | 9397 | −228 | 733 | −325 | O |
| HETATM | 3166 | O | HOH | | 446 | −0.438 | 34.830 | 21.966 | 1.00 | 76.69 | | O |
| ANISOU | 3166 | O | HOH | | 446 | 11316 | 7627 | 10198 | 762 | −1465 | 911 | O |
| HETATM | 3167 | O | HOH | | 447 | −7.505 | 12.096 | 32.851 | 1.00 | 62.92 | | O |
| ANISOU | 3167 | O | HOH | | 447 | 8162 | 8656 | 7087 | −1524 | 1162 | 200 | O |
| HETATM | 3168 | O | HOH | | 448 | 18.699 | −5.610 | −8.087 | 1.00 | 61.07 | | O |
| ANISOU | 3168 | O | HOH | | 448 | 8547 | 6458 | 8199 | −44 | −1549 | 1091 | O |
| HETATM | 3169 | O | HOH | | 449 | 4.418 | 1.224 | 27.880 | 1.00 | 48.87 | | O |
| ANISOU | 3169 | O | HOH | | 449 | 7308 | 5380 | 5881 | −777 | −1100 | 2366 | O |
| HETATM | 3170 | O | HOH | | 450 | −4.699 | 27.479 | 8.319 | 1.00 | 45.92 | | O |
| ANISOU | 3170 | O | HOH | | 450 | 5567 | 5491 | 6391 | 764 | −1472 | −1137 | O |
| HETATM | 3171 | O | HOH | | 451 | 29.301 | −10.194 | 8.620 | 1.00 | 53.48 | | O |
| ANISOU | 3171 | O | HOH | | 451 | 7766 | 7580 | 4974 | 293 | −111 | 37 | O |
| HETATM | 3172 | O | HOH | | 452 | 10.823 | 3.130 | −29.102 | 1.00 | 54.34 | | O |
| ANISOU | 3172 | O | HOH | | 452 | 6506 | 6609 | 7533 | −690 | 213 | −401 | O |
| HETATM | 3173 | O | HOH | | 453 | −11.291 | 10.423 | −12.654 | 1.00 | 52.78 | | O |
| ANISOU | 3173 | O | HOH | | 453 | 6250 | 7011 | 6792 | 1859 | −803 | −208 | O |
| HETATM | 3174 | O | HOH | | 454 | 0.219 | 8.119 | −2.571 | 1.00 | 39.27 | | O |
| ANISOU | 3174 | O | HOH | | 454 | 4184 | 6633 | 4102 | −1058 | 43 | −113 | O |
| HETATM | 3175 | O | HOH | | 455 | 14.114 | −12.640 | −16.647 | 1.00 | 59.48 | | O |
| ANISOU | 3175 | O | HOH | | 455 | 3981 | 9044 | 9575 | 3317 | 759 | −1954 | O |
| HETATM | 3176 | O | HOH | | 456 | 2.884 | −16.174 | 0.442 | 1.00 | 42.30 | | O |
| ANISOU | 3176 | O | HOH | | 456 | 3034 | 7892 | 5145 | 611 | −404 | −763 | O |
| HETATM | 3177 | O | HOH | | 457 | 3.232 | 14.034 | −7.198 | 1.00 | 56.91 | | O |
| ANISOU | 3177 | O | HOH | | 457 | 7527 | 6661 | 7436 | 1953 | −1040 | −747 | O |
| HETATM | 3178 | O | HOH | | 458 | 10.108 | 28.250 | 6.631 | 1.00 | 42.33 | | O |
| ANISOU | 3178 | O | HOH | | 458 | 6124 | 5605 | 4353 | −121 | 165 | 1990 | O |
| HETATM | 3179 | O | HOH | | 459 | 5.062 | 13.436 | 1.308 | 1.00 | 60.01 | | O |
| ANISOU | 3179 | O | HOH | | 459 | 8222 | 8617 | 5964 | −233 | −641 | 498 | O |
| HETATM | 3180 | O | HOH | | 460 | −6.860 | 33.817 | 20.822 | 1.00 | 48.41 | | O |
| ANISOU | 3180 | O | HOH | | 460 | 6445 | 6837 | 5112 | 567 | −363 | −675 | O |
| HETATM | 3181 | O | HOH | | 461 | 10.476 | 31.743 | 13.651 | 1.00 | 53.99 | | O |
| ANISOU | 3181 | O | HOH | | 461 | 7302 | 6552 | 6659 | 263 | −620 | 1506 | O |
| HETATM | 3182 | O | HOH | | 462 | 17.087 | 18.185 | 13.678 | 1.00 | 51.70 | | O |
| ANISOU | 3182 | O | HOH | | 462 | 5989 | 7619 | 6036 | −1221 | 1753 | −846 | O |
| HETATM | 3183 | O | HOH | | 463 | 0.391 | −11.590 | 9.118 | 1.00 | 63.23 | | O |
| ANISOU | 3183 | O | HOH | | 463 | 8630 | 7863 | 7531 | −33 | −956 | 1290 | O |
| HETATM | 3184 | O | HOH | | 464 | 8.547 | 7.667 | −13.893 | 1.00 | 30.27 | | O |
| ANISOU | 3184 | O | HOH | | 464 | 3585 | 3364 | 4554 | −1012 | 151 | −475 | O |
| HETATM | 3185 | O | HOH | | 465 | 8.902 | −5.008 | 14.281 | 1.00 | 59.23 | | O |
| ANISOU | 3185 | O | HOH | | 465 | 8662 | 4746 | 9096 | −2177 | −877 | 2551 | O |
| HETATM | 3186 | O | HOH | | 466 | 14.306 | −7.941 | 5.666 | 1.00 | 37.02 | | O |
| ANISOU | 3186 | O | HOH | | 466 | 4981 | 3013 | 6071 | 247 | −1495 | −1110 | O |
| HETATM | 3187 | O | HOH | | 467 | 12.586 | −1.159 | 21.256 | 1.00 | 66.10 | | O |
| ANISOU | 3187 | O | HOH | | 467 | 8063 | 9055 | 7997 | 21 | 652 | 387 | O |
| HETATM | 3188 | O | HOH | | 468 | 1.602 | 14.965 | −5.429 | 1.00 | 55.49 | | O |
| ANISOU | 3188 | O | HOH | | 468 | 7372 | 6249 | 7462 | −2610 | 928 | 447 | O |
| HETATM | 3189 | O | HOH | | 469 | 10.679 | 7.344 | 0.076 | 1.00 | 41.12 | | O |
| ANISOU | 3189 | O | HOH | | 469 | 6341 | 4400 | 4882 | −983 | −147 | 1538 | O |
| HETATM | 3190 | O | HOH | | 470 | −6.420 | −0.762 | −24.528 | 1.00 | 54.85 | | O |
| ANISOU | 3190 | O | HOH | | 470 | 6931 | 6390 | 7518 | 2094 | −1083 | 51 | O |
| HETATM | 3191 | O | HOH | | 471 | −5.265 | −2.344 | 33.048 | 1.00 | 54.17 | | O |
| ANISOU | 3191 | O | HOH | | 471 | 6562 | 7074 | 6945 | −1857 | 1064 | −58 | O |
| HETATM | 3192 | O | HOH | | 472 | −0.216 | 2.349 | −27.459 | 1.00 | 99.44 | | O |
| ANISOU | 3192 | O | HOH | | 472 | 13656 | 11536 | 12592 | 182 | −836 | 573 | O |
| HETATM | 3193 | O | HOH | | 473 | −3.852 | 10.724 | 5.829 | 1.00 | 58.14 | | O |
| ANISOU | 3193 | O | HOH | | 473 | 7774 | 6637 | 7679 | 1951 | −1501 | −72 | O |
| HETATM | 3194 | O | HOH | | 474 | 11.248 | 25.775 | 5.974 | 1.00 | 37.46 | | O |
| ANISOU | 3194 | O | HOH | | 474 | 5387 | 4317 | 4529 | −1566 | 918 | 1337 | O |
| HETATM | 3195 | O | HOH | | 475 | 2.683 | −5.338 | −30.078 | 1.00 | 56.88 | | O |
| ANISOU | 3195 | O | HOH | | 475 | 7228 | 7538 | 6848 | −686 | 371 | −1065 | O |
| HETATM | 3196 | O | HOH | | 476 | 10.341 | −1.043 | 22.919 | 1.00 | 59.26 | | O |
| ANISOU | 3196 | O | HOH | | 476 | 5904 | 8018 | 8594 | 2703 | −1695 | −149 | O |
| HETATM | 3197 | O | HOH | | 477 | 19.955 | 15.073 | 14.430 | 1.00 | 43.85 | | O |
| ANISOU | 3197 | O | HOH | | 477 | 3578 | 6514 | 6568 | −1412 | 457 | −637 | O |
| HETATM | 3198 | O | HOH | | 478 | −16.506 | −2.143 | 4.327 | 1.00 | 60.41 | | O |
| ANISOU | 3198 | O | HOH | | 478 | 8078 | 7285 | 7588 | −1410 | −375 | 301 | O |
| HETATM | 3199 | O | HOH | | 479 | 21.410 | 16.815 | 18.994 | 1.00 | 45.99 | | O |
| ANISOU | 3199 | O | HOH | | 479 | 5380 | 5117 | 6978 | 37 | 480 | −845 | O |
| HETATM | 3200 | O | HOH | | 480 | 12.054 | 13.110 | −0.051 | 1.00 | 60.04 | | O |
| ANISOU | 3200 | O | HOH | | 480 | 7719 | 9764 | 5330 | −1779 | 2397 | 263 | O |
| HETATM | 3201 | O | HOH | | 481 | −8.771 | 12.340 | −16.358 | 1.00 | 61.06 | | O |
| ANISOU | 3201 | O | HOH | | 481 | 8519 | 6753 | 7929 | −2467 | −66 | 2481 | O |
| HETATM | 3202 | O | HOH | | 482 | 14.503 | 8.281 | 1.629 | 1.00 | 61.56 | | O |
| ANISOU | 3202 | O | HOH | | 482 | 7910 | 7606 | 7872 | 217 | 299 | −1016 | O |
| HETATM | 3203 | O | HOH | | 483 | 5.729 | −2.330 | 21.945 | 1.00 | 44.83 | | O |
| ANISOU | 3203 | O | HOH | | 483 | 6239 | 5276 | 5517 | −1561 | −2389 | 1087 | O |
| HETATM | 3204 | O | HOH | | 484 | 9.522 | −14.720 | −22.450 | 1.00 | 46.14 | | O |
| ANISOU | 3204 | O | HOH | | 484 | 3757 | 6212 | 7563 | 1387 | 967 | −1537 | O |
| HETATM | 3205 | O | HOH | | 485 | 4.009 | −19.739 | 2.171 | 1.00 | 36.76 | | O |
| ANISOU | 3205 | O | HOH | | 485 | 4519 | 4408 | 5038 | −2616 | −996 | 2724 | O |
| HETATM | 3206 | O | HOH | | 486 | −5.699 | 20.492 | 4.169 | 1.00 | 44.40 | | O |
| ANISOU | 3206 | O | HOH | | 486 | 6484 | 5347 | 5038 | −301 | −403 | 384 | O |
| HETATM | 3207 | O | HOH | | 487 | 1.448 | 32.934 | 21.812 | 1.00 | 40.79 | | O |
| ANISOU | 3207 | O | HOH | | 487 | 4288 | 5196 | 6015 | −1991 | 846 | −800 | O |
| HETATM | 3208 | O | HOH | | 488 | 17.143 | −4.111 | −6.334 | 1.00 | 46.96 | | O |
| ANISOU | 3208 | O | HOH | | 488 | 6516 | 5151 | 6176 | 79 | −1796 | 583 | O |
| HETATM | 3209 | O | HOH | | 489 | 20.972 | −1.778 | −9.715 | 1.00 | 42.99 | | O |
| ANISOU | 3209 | O | HOH | | 489 | 4571 | 6651 | 5112 | 1382 | 331 | −1693 | O |
| HETATM | 3210 | O | HOH | | 490 | 6.343 | 8.508 | −25.572 | 1.00 | 45.20 | | O |
| ANISOU | 3210 | O | HOH | | 490 | 6182 | 4951 | 6039 | 906 | −1601 | −1351 | O |
| HETATM | 3211 | O | HOH | | 491 | 18.187 | 18.600 | 5.207 | 1.00 | 52.97 | | O |
| ANISOU | 3211 | O | HOH | | 491 | 5268 | 7345 | 7514 | 561 | 1507 | −1809 | O |
| HETATM | 3212 | O | HOH | | 492 | 1.335 | 35.203 | 30.487 | 1.00 | 81.27 | | O |
| ANISOU | 3212 | O | HOH | | 492 | 11056 | 9143 | 10679 | 449 | −972 | 463 | O |
| HETATM | 3213 | O | HOH | | 493 | 25.882 | −7.093 | 16.686 | 1.00 | 48.68 | | O |
| ANISOU | 3213 | O | HOH | | 493 | 7388 | 5020 | 6090 | −1490 | 1937 | 722 | O |
| HETATM | 3214 | O | HOH | | 494 | −15.605 | 19.157 | 14.795 | 1.00 | 45.95 | | O |
| ANISOU | 3214 | O | HOH | | 494 | 6307 | 6024 | 5129 | 813 | −1118 | 916 | O |
| HETATM | 3215 | O | HOH | | 495 | −4.015 | 17.981 | 3.705 | 1.00 | 44.21 | | O |
| ANISOU | 3215 | O | HOH | | 495 | 6661 | 5092 | 5044 | 416 | −2629 | −701 | O |
| HETATM | 3216 | O | HOH | | 496 | −2.421 | 0.088 | −26.159 | 1.00 | 41.62 | | O |
| ANISOU | 3216 | O | HOH | | 496 | 6834 | 5246 | 3733 | −1274 | −641 | 318 | O |
| HETATM | 3217 | O | HOH | | 497 | −15.840 | 1.240 | −7.595 | 1.00 | 44.82 | | O |
| ANISOU | 3217 | O | HOH | | 497 | 4518 | 6735 | 5778 | 2052 | 370 | −712 | O |
| HETATM | 3218 | O | HOH | | 498 | −14.382 | −15.093 | 0.886 | 1.00 | 53.69 | | O |
| ANISOU | 3218 | O | HOH | | 498 | 6895 | 7516 | 5991 | 433 | 1162 | −1713 | O |
| HETATM | 3219 | O | HOH | | 499 | 5.036 | 32.031 | 6.735 | 1.00 | 43.03 | | O |
| ANISOU | 3219 | O | HOH | | 499 | 6485 | 5429 | 4436 | 1215 | −1343 | −658 | O |
| HETATM | 3220 | O | HOH | | 500 | −19.290 | 19.516 | 15.863 | 1.00 | 97.26 | | O |
| ANISOU | 3220 | O | HOH | | 500 | 11991 | 12598 | 12366 | 1085 | −134 | −943 | O |
| HETATM | 3221 | O | HOH | | 501 | 10.737 | 30.909 | 15.843 | 1.00 | 79.16 | | O |
| ANISOU | 3221 | O | HOH | | 501 | 9731 | 10210 | 10134 | 825 | 247 | −913 | O |
| HETATM | 3222 | O | HOH | | 502 | 8.190 | −2.352 | 33.238 | 1.00 | 64.86 | | O |
| ANISOU | 3222 | O | HOH | | 502 | 9570 | 7649 | 7427 | −687 | −340 | 496 | O |
| HETATM | 3223 | O | HOH | | 503 | −11.525 | 10.611 | 13.283 | 1.00 | 60.18 | | O |
| ANISOU | 3223 | O | HOH | | 503 | 7961 | 7727 | 7176 | 272 | −1670 | −955 | O |
| HETATM | 3224 | O | HOH | | 504 | 3.276 | 2.608 | −29.291 | 1.00 | 38.50 | | O |
| ANISOU | 3224 | O | HOH | | 504 | 4020 | 5067 | 5542 | −1156 | −1006 | 574 | O |
| HETATM | 3225 | O | HOH | | 505 | −10.044 | 16.909 | 28.948 | 1.00 | 50.10 | | O |
| ANISOU | 3225 | O | HOH | | 505 | 4407 | 6995 | 7632 | −2876 | 2424 | −406 | O |
| HETATM | 3226 | O | HOH | | 506 | −14.380 | 14.763 | 16.189 | 1.00 | 40.18 | | O |
| ANISOU | 3226 | O | HOH | | 506 | 3825 | 5221 | 6218 | 675 | −377 | −773 | O |
| HETATM | 3227 | O | HOH | | 507 | 1.305 | −1.278 | −28.757 | 1.00 | 47.79 | | O |
| ANISOU | 3227 | O | HOH | | 507 | 3933 | 7995 | 6228 | 1049 | −1245 | −1325 | O |
| HETATM | 3228 | O | HOH | | 508 | 17.731 | 10.087 | 4.475 | 1.00 | 58.59 | | O |
| ANISOU | 3228 | O | HOH | | 508 | 7587 | 7755 | 6921 | 43 | 111 | 787 | O |
| END | | | | | | | | | | | | |

TABLE 5
Summary of Data Collection, Phasing and Refinement
for the Rab9-GppNHp Complex

| | |
|---|---|
| Data and Phasing Statistics | |
| Space group | P2₁2₁2₁ |
| Unit cell: a, b, c (Å) | 56.24, 76.60, 174.35 |
| Wavelength (Å) | 1.00 |
| Resolution (Å) | 1.73 |
| Reflections (total/unique) | 217,263/75,144 |
| Completeness (%)^{a} | 94.5 (69.1) |
| I/σ^{a} | 10.1 (2.6) |
| R(I)_{merge} (%)^{a} | 7.0 (21.7) |
| Molecular replacement model | Rab9-GDP dimer |
| Refinement Statistics | |
| Resolution range (Å) | 20-1.73 |
| Number of reflections | 65,461 |
| Number of atoms | protein/GppNHp/Mg²⁺/ |
| | water, 5,428/128/4/495 |
| R factors (%) | R_{work}/R_{free}, 19.4/22.3 |
| R.m.s. deviation of bonds | length/angle, 0.005 Å/1.2° |

^{a}Values in parentheses are for the highest resolution shell.

TABLE 6
Atomic Coordinate and Structure Factor Data for the Rab9-GppNHp Complex

CRYST1 56.239 76.600 174.354 90.00 90.00 90.00 P 21 21 21 16

| | | | | | | | | | | | |
|---|---|---|---|---|---|---|---|---|---|---|---|
| ATOM | 1 | CB | SER | A | 6 | 40.163 | 25.978 | 60.319 | 1.00 | 24.65 | A |
| ATOM | 2 | OG | SER | A | 6 | 39.630 | 24.786 | 60.882 | 1.00 | 30.01 | A |
| ATOM | 3 | C | SER | A | 6 | 38.292 | 27.486 | 61.036 | 1.00 | 18.50 | A |
| ATOM | 4 | O | SER | A | 6 | 38.608 | 28.581 | 61.513 | 1.00 | 16.36 | A |
| ATOM | 5 | N | SER | A | 6 | 39.610 | 28.000 | 59.000 | 1.00 | 21.73 | A |
| ATOM | 6 | CA | SER | A | 6 | 39.044 | 26.905 | 59.837 | 1.00 | 20.85 | A |
| ATOM | 7 | N | LEU | A | 7 | 37.300 | 26.743 | 61.516 | 1.00 | 15.25 | A |
| ATOM | 8 | CA | LEU | A | 7 | 36.482 | 27.177 | 62.645 | 1.00 | 13.56 | A |
| ATOM | 9 | CB | LEU | A | 7 | 35.031 | 26.734 | 62.434 | 1.00 | 12.76 | A |
| ATOM | 10 | CG | LEU | A | 7 | 33.991 | 27.205 | 63.456 | 1.00 | 12.09 | A |
| ATOM | 11 | CD1 | LEU | A | 7 | 33.806 | 28.709 | 63.336 | 1.00 | 10.42 | A |
| ATOM | 12 | CD2 | LEU | A | 7 | 32.669 | 26.492 | 63.202 | 1.00 | 12.23 | A |
| ATOM | 13 | C | LEU | A | 7 | 36.999 | 26.605 | 63.961 | 1.00 | 12.93 | A |
| ATOM | 14 | O | LEU | A | 7 | 36.939 | 25.399 | 64.187 | 1.00 | 14.63 | A |
| ATOM | 15 | N | PHE | A | 8 | 37.506 | 27.481 | 64.821 | 1.00 | 12.21 | A |
| ATOM | 16 | CA | PHE | A | 8 | 38.036 | 27.081 | 66.124 | 1.00 | 10.95 | A |
| ATOM | 17 | CB | PHE | A | 8 | 39.394 | 27.741 | 66.378 | 1.00 | 10.84 | A |
| ATOM | 18 | CG | PHE | A | 8 | 40.518 | 27.182 | 65.551 | 1.00 | 11.42 | A |
| ATOM | 19 | CD1 | PHE | A | 8 | 41.810 | 27.672 | 65.706 | 1.00 | 11.20 | A |
| ATOM | 20 | CD2 | PHE | A | 8 | 40.296 | 26.159 | 64.634 | 1.00 | 11.73 | A |
| ATOM | 21 | CE1 | PHE | A | 8 | 42.870 | 27.152 | 64.959 | 1.00 | 12.09 | A |
| ATOM | 22 | CE2 | PHE | A | 8 | 41.351 | 25.633 | 63.883 | 1.00 | 13.75 | A |
| ATOM | 23 | CZ | PHE | A | 8 | 42.636 | 26.131 | 64.047 | 1.00 | 11.16 | A |
| ATOM | 24 | C | PHE | A | 8 | 37.078 | 27.508 | 67.227 | 1.00 | 10.29 | A |
| ATOM | 25 | O | PHE | A | 8 | 36.862 | 28.698 | 67.442 | 1.00 | 11.07 | A |
| ATOM | 26 | N | LYS | A | 9 | 36.516 | 26.538 | 67.936 | 1.00 | 8.66 | A |
| ATOM | 27 | CA | LYS | A | 9 | 35.581 | 26.841 | 69.011 | 1.00 | 8.15 | A |
| ATOM | 28 | CB | LYS | A | 9 | 34.556 | 25.716 | 69.142 | 1.00 | 7.59 | A |
| ATOM | 29 | CG | LYS | A | 9 | 33.550 | 25.912 | 70.259 | 1.00 | 5.67 | A |
| ATOM | 30 | CD | LYS | A | 9 | 32.521 | 24.796 | 70.246 | 1.00 | 8.59 | A |
| ATOM | 31 | CE | LYS | A | 9 | 31.566 | 24.893 | 71.425 | 1.00 | 11.11 | A |
| ATOM | 32 | NZ | LYS | A | 9 | 30.582 | 23.772 | 71.399 | 1.00 | 12.43 | A |
| ATOM | 33 | C | LYS | A | 9 | 36.328 | 27.025 | 70.325 | 1.00 | 7.61 | A |
| ATOM | 34 | O | LYS | A | 9 | 37.019 | 26.114 | 70.791 | 1.00 | 7.67 | A |
| ATOM | 35 | N | VAL | A | 10 | 36.186 | 28.209 | 70.911 | 1.00 | 7.40 | A |
| ATOM | 36 | CA | VAL | A | 10 | 36.849 | 28.535 | 72.167 | 1.00 | 8.17 | A |
| ATOM | 37 | CB | VAL | A | 10 | 37.794 | 29.735 | 71.992 | 1.00 | 9.75 | A |
| ATOM | 38 | CG1 | VAL | A | 10 | 38.496 | 30.042 | 73.307 | 1.00 | 10.73 | A |
| ATOM | 39 | CG2 | VAL | A | 10 | 38.815 | 29.429 | 70.903 | 1.00 | 11.04 | A |
| ATOM | 40 | C | VAL | A | 10 | 35.788 | 28.867 | 73.202 | 1.00 | 7.60 | A |
| ATOM | 41 | O | VAL | A | 10 | 35.011 | 29.803 | 73.031 | 1.00 | 8.56 | A |
| ATOM | 42 | N | ILE | A | 11 | 35.760 | 28.098 | 74.281 | 1.00 | 6.61 | A |
| ATOM | 43 | CA | ILE | A | 11 | 34.754 | 28.307 | 75.310 | 1.00 | 7.85 | A |
| ATOM | 44 | CB | ILE | A | 11 | 34.072 | 26.955 | 75.642 | 1.00 | 6.65 | A |
| ATOM | 45 | CG2 | ILE | A | 11 | 35.051 | 26.036 | 76.350 | 1.00 | 10.37 | A |
| ATOM | 46 | CG1 | ILE | A | 11 | 32.822 | 27.175 | 76.494 | 1.00 | 10.64 | A |
| ATOM | 47 | CD1 | ILE | A | 11 | 31.949 | 25.933 | 76.603 | 1.00 | 12.16 | A |
| ATOM | 48 | C | ILE | A | 11 | 35.316 | 28.961 | 76.576 | 1.00 | 7.54 | A |
| ATOM | 49 | O | ILE | A | 11 | 36.394 | 28.610 | 77.052 | 1.00 | 7.61 | A |
| ATOM | 50 | N | LEU | A | 12 | 34.576 | 29.933 | 77.100 | 1.00 | 6.86 | A |
| ATOM | 51 | CA | LEU | A | 12 | 34.964 | 30.645 | 78.311 | 1.00 | 8.17 | A |
| ATOM | 52 | CB | LEU | A | 12 | 34.574 | 32.119 | 78.208 | 1.00 | 9.45 | A |
| ATOM | 53 | CG | LEU | A | 12 | 35.255 | 32.962 | 77.133 | 1.00 | 11.11 | A |
| ATOM | 54 | CD1 | LEU | A | 12 | 34.498 | 34.275 | 76.947 | 1.00 | 11.52 | A |
| ATOM | 55 | CD2 | LEU | A | 12 | 36.687 | 33.216 | 77.542 | 1.00 | 13.77 | A |
| ATOM | 56 | C | LEU | A | 12 | 34.255 | 30.045 | 79.512 | 1.00 | 7.37 | A |
| ATOM | 57 | O | LEU | A | 12 | 33.024 | 30.053 | 79.577 | 1.00 | 7.57 | A |
| ATOM | 58 | N | LEU | A | 13 | 35.029 | 29.521 | 80.456 | 1.00 | 6.99 | A |
| ATOM | 59 | CA | LEU | A | 13 | 34.459 | 28.944 | 81.668 | 1.00 | 8.64 | A |
| ATOM | 60 | CB | LEU | A | 13 | 34.798 | 27.455 | 81.772 | 1.00 | 8.29 | A |
| ATOM | 61 | CG | LEU | A | 13 | 34.239 | 26.547 | 80.674 | 1.00 | 11.64 | A |
| ATOM | 62 | CD1 | LEU | A | 13 | 34.528 | 25.094 | 81.033 | 1.00 | 11.58 | A |
| ATOM | 63 | CD2 | LEU | A | 13 | 32.738 | 26.756 | 80.534 | 1.00 | 11.13 | A |
| ATOM | 64 | C | LEU | A | 13 | 35.013 | 29.691 | 82.875 | 1.00 | 7.04 | A |
| ATOM | 65 | O | LEU | A | 13 | 36.049 | 30.346 | 82.775 | 1.00 | 8.93 | A |
| ATOM | 66 | N | GLY | A | 14 | 34.319 | 29.595 | 84.006 | 1.00 | 6.85 | A |
| ATOM | 67 | CA | GLY | A | 14 | 34.757 | 30.280 | 85.212 | 1.00 | 5.87 | A |
| ATOM | 68 | C | GLY | A | 14 | 33.574 | 30.753 | 86.043 | 1.00 | 6.09 | A |
| ATOM | 69 | O | GLY | A | 14 | 32.472 | 30.931 | 85.519 | 1.00 | 6.68 | A |
| ATOM | 70 | N | ASP | A | 15 | 33.799 | 30.971 | 87.337 | 1.00 | 6.23 | A |
| ATOM | 71 | CA | ASP | A | 15 | 32.736 | 31.398 | 88.244 | 1.00 | 6.95 | A |
| ATOM | 72 | CB | ASP | A | 15 | 33.296 | 31.657 | 89.641 | 1.00 | 7.59 | A |
| ATOM | 73 | CG | ASP | A | 15 | 33.664 | 30.383 | 90.378 | 1.00 | 10.19 | A |
| ATOM | 74 | OD1 | ASP | A | 15 | 33.472 | 29.277 | 89.824 | 1.00 | 9.57 | A |
| ATOM | 75 | OD2 | ASP | A | 15 | 34.142 | 30.497 | 91.526 | 1.00 | 11.75 | A |
| ATOM | 76 | C | ASP | A | 15 | 31.990 | 32.646 | 87.799 | 1.00 | 5.99 | A |
| ATOM | 77 | O | ASP | A | 15 | 32.543 | 33.516 | 87.124 | 1.00 | 7.75 | A |
| ATOM | 78 | N | GLY | A | 16 | 30.728 | 32.733 | 88.195 | 1.00 | 4.79 | A |
| ATOM | 79 | CA | GLY | A | 16 | 29.956 | 33.907 | 87.847 | 1.00 | 5.74 | A |
| ATOM | 80 | C | GLY | A | 16 | 30.657 | 35.106 | 88.452 | 1.00 | 6.93 | A |
| ATOM | 81 | O | GLY | A | 16 | 31.160 | 35.034 | 89.580 | 1.00 | 8.28 | A |
| ATOM | 82 | N | GLY | A | 17 | 30.733 | 36.192 | 87.688 | 1.00 | 6.86 | A |
| ATOM | 83 | CA | GLY | A | 17 | 31.362 | 37.406 | 88.178 | 1.00 | 5.80 | A |
| ATOM | 84 | C | GLY | A | 17 | 32.836 | 37.633 | 87.884 | 1.00 | 6.54 | A |
| ATOM | 85 | O | GLY | A | 17 | 33.344 | 38.720 | 88.156 | 1.00 | 8.66 | A |
| ATOM | 86 | N | VAL | A | 18 | 33.532 | 36.642 | 87.328 | 1.00 | 5.27 | A |
| ATOM | 87 | CA | VAL | A | 18 | 34.955 | 36.813 | 87.048 | 1.00 | 5.42 | A |
| ATOM | 88 | CB | VAL | A | 18 | 35.668 | 35.457 | 86.851 | 1.00 | 5.50 | A |
| ATOM | 89 | CG1 | VAL | A | 18 | 35.479 | 34.595 | 88.093 | 1.00 | 8.28 | A |
| ATOM | 90 | CG2 | VAL | A | 18 | 35.126 | 34.744 | 85.614 | 1.00 | 5.69 | A |
| ATOM | 91 | C | VAL | A | 18 | 35.243 | 37.708 | 85.851 | 1.00 | 4.20 | A |
| ATOM | 92 | O | VAL | A | 18 | 36.360 | 38.206 | 85.707 | 1.00 | 5.93 | A |
| ATOM | 93 | N | GLY | A | 19 | 34.243 | 37.912 | 84.996 | 1.00 | 4.66 | A |
| ATOM | 94 | CA | GLY | A | 19 | 34.431 | 38.786 | 83.845 | 1.00 | 3.87 | A |
| ATOM | 95 | C | GLY | A | 19 | 34.423 | 38.148 | 82.466 | 1.00 | 4.83 | A |
| ATOM | 96 | O | GLY | A | 19 | 34.919 | 38.745 | 81.510 | 1.00 | 4.90 | A |
| ATOM | 97 | N | LYS | A | 20 | 33.861 | 36.948 | 82.344 | 1.00 | 4.21 | A |
| ATOM | 98 | CA | LYS | A | 20 | 33.813 | 36.270 | 81.045 | 1.00 | 3.82 | A |
| ATOM | 99 | CB | LYS | A | 20 | 33.126 | 34.910 | 81.185 | 1.00 | 4.40 | A |
| ATOM | 100 | CG | LYS | A | 20 | 33.890 | 33.950 | 82.087 | 1.00 | 3.50 | A |
| ATOM | 101 | CD | LYS | A | 20 | 33.175 | 32.608 | 82.254 | 1.00 | 3.57 | A |
| ATOM | 102 | CE | LYS | A | 20 | 31.766 | 32.772 | 82.826 | 1.00 | 5.62 | A |
| ATOM | 103 | NZ | LYS | A | 20 | 31.723 | 33.479 | 84.132 | 1.00 | 5.41 | A |
| ATOM | 104 | C | LYS | A | 20 | 33.098 | 37.100 | 79.983 | 1.00 | 4.27 | A |
| ATOM | 105 | O | LYS | A | 20 | 33.627 | 37.310 | 78.887 | 1.00 | 7.10 | A |
| ATOM | 106 | N | SER | A | 21 | 31.898 | 37.574 | 80.296 | 1.00 | 4.93 | A |
| ATOM | 107 | CA | SER | A | 21 | 31.160 | 38.377 | 79.322 | 1.00 | 5.21 | A |
| ATOM | 108 | CB | SER | A | 21 | 29.771 | 38.738 | 79.848 | 1.00 | 6.34 | A |
| ATOM | 109 | OG | SER | A | 21 | 28.971 | 37.579 | 79.964 | 1.00 | 4.11 | A |
| ATOM | 110 | C | SER | A | 21 | 31.911 | 39.654 | 78.975 | 1.00 | 5.13 | A |
| ATOM | 111 | O | SER | A | 21 | 31.925 | 40.079 | 77.813 | 1.00 | 5.61 | A |
| ATOM | 112 | N | SER | A | 22 | 32.526 | 40.272 | 79.979 | 1.00 | 3.56 | A |
| ATOM | 113 | CA | SER | A | 22 | 33.269 | 41.506 | 79.751 | 1.00 | 4.48 | A |
| ATOM | 114 | CB | SER | A | 22 | 33.695 | 42.127 | 81.083 | 1.00 | 5.48 | A |
| ATOM | 115 | OG | SER | A | 22 | 32.564 | 42.516 | 81.848 | 1.00 | 5.66 | A |
| ATOM | 116 | C | SER | A | 22 | 34.493 | 41.231 | 78.873 | 1.00 | 5.04 | A |
| ATOM | 117 | O | SER | A | 22 | 34.831 | 42.028 | 78.000 | 1.00 | 4.62 | A |
| ATOM | 118 | N | LEU | A | 23 | 35.165 | 40.105 | 79.100 | 1.00 | 4.80 | A |
| ATOM | 119 | CA | LEU | A | 23 | 36.331 | 39.783 | 78.282 | 1.00 | 5.52 | A |
| ATOM | 120 | CB | LEU | A | 23 | 37.030 | 38.520 | 78.806 | 1.00 | 5.32 | A |
| ATOM | 121 | CG | LEU | A | 23 | 37.779 | 38.756 | 80.122 | 1.00 | 6.74 | A |
| ATOM | 122 | CD1 | LEU | A | 23 | 38.153 | 37.427 | 80.773 | 1.00 | 6.78 | A |
| ATOM | 123 | CD2 | LEU | A | 23 | 39.023 | 39.591 | 79.841 | 1.00 | 7.78 | A |
| ATOM | 124 | C | LEU | A | 23 | 35.932 | 39.592 | 76.819 | 1.00 | 6.18 | A |
| ATOM | 125 | O | LEU | A | 23 | 36.601 | 40.097 | 75.912 | 1.00 | 4.12 | A |
| ATOM | 126 | N | MET | A | 24 | 34.837 | 38.874 | 76.592 | 1.00 | 6.07 | A |
| ATOM | 127 | CA | MET | A | 24 | 34.367 | 38.620 | 75.237 | 1.00 | 8.29 | A |
| ATOM | 128 | CB | MET | A | 24 | 33.154 | 37.686 | 75.259 | 1.00 | 8.61 | A |
| ATOM | 129 | CG | MET | A | 24 | 32.576 | 37.394 | 73.884 | 1.00 | 12.84 | A |
| ATOM | 130 | SD | MET | A | 24 | 31.273 | 36.150 | 73.944 | 1.00 | 16.80 | A |
| ATOM | 131 | CE | MET | A | 24 | 29.881 | 37.106 | 74.439 | 1.00 | 18.74 | A |
| ATOM | 132 | C | MET | A | 24 | 33.983 | 39.922 | 74.549 | 1.00 | 7.81 | A |
| ATOM | 133 | O | MET | A | 24 | 34.373 | 40.173 | 73.409 | 1.00 | 8.04 | A |
| ATOM | 134 | N | ASN | A | 25 | 33.223 | 40.756 | 75.251 | 1.00 | 8.44 | A |
| ATOM | 135 | CA | ASN | A | 25 | 32.785 | 42.017 | 74.674 | 1.00 | 8.23 | A |
| ATOM | 136 | CB | ASN | A | 25 | 31.755 | 42.684 | 75.591 | 1.00 | 9.61 | A |
| ATOM | 137 | CG | ASN | A | 25 | 31.032 | 43.827 | 74.912 | 1.00 | 13.46 | A |
| ATOM | 138 | OD1 | ASN | A | 25 | 30.739 | 43.764 | 73.716 | 1.00 | 14.08 | A |
| ATOM | 139 | ND2 | ASN | A | 25 | 30.728 | 44.873 | 75.671 | 1.00 | 15.24 | A |
| ATOM | 140 | C | ASN | A | 25 | 33.956 | 42.961 | 74.408 | 1.00 | 7.23 | A |
| ATOM | 141 | O | ASN | A | 25 | 33.966 | 43.679 | 73.404 | 1.00 | 7.22 | A |
| ATOM | 142 | N | ARG | A | 26 | 34.947 | 42.954 | 75.292 | 1.00 | 5.84 | A |
| ATOM | 143 | CA | ARG | A | 26 | 36.107 | 43.818 | 75.110 | 1.00 | 5.47 | A |
| ATOM | 144 | CB | ARG | A | 26 | 37.026 | 43.764 | 76.337 | 1.00 | 5.54 | A |
| ATOM | 145 | CG | ARG | A | 26 | 38.134 | 44.801 | 76.283 | 1.00 | 10.19 | A |
| ATOM | 146 | CD | ARG | A | 26 | 37.527 | 46.190 | 76.452 | 1.00 | 9.87 | A |
| ATOM | 147 | NE | ARG | A | 26 | 38.388 | 47.256 | 75.954 | 1.00 | 14.85 | A |
| ATOM | 148 | CZ | ARG | A | 26 | 38.772 | 48.301 | 76.676 | 1.00 | 15.39 | A |
| ATOM | 149 | NH1 | ARG | A | 26 | 38.379 | 48.420 | 77.940 | 1.00 | 15.31 | A |
| ATOM | 150 | NH2 | ARG | A | 26 | 39.529 | 49.240 | 76.127 | 1.00 | 17.26 | A |
| ATOM | 151 | C | ARG | A | 26 | 36.890 | 43.386 | 73.874 | 1.00 | 7.04 | A |
| ATOM | 152 | O | ARG | A | 26 | 37.357 | 44.219 | 73.097 | 1.00 | 6.47 | A |
| ATOM | 153 | N | TYR | A | 27 | 37.031 | 42.079 | 73.683 | 1.00 | 5.01 | A |
| ATOM | 154 | CA | TYR | A | 27 | 37.772 | 41.584 | 72.528 | 1.00 | 6.92 | A |
| ATOM | 155 | CB | TYR | A | 27 | 37.998 | 40.081 | 72.671 | 1.00 | 5.75 | A |
| ATOM | 156 | CG | TYR | A | 27 | 38.900 | 39.476 | 71.615 | 1.00 | 6.66 | A |
| ATOM | 157 | CD1 | TYR | A | 27 | 40.160 | 40.019 | 71.345 | 1.00 | 6.32 | A |
| ATOM | 158 | CE1 | TYR | A | 27 | 41.025 | 39.412 | 70.426 | 1.00 | 7.80 | A |
| ATOM | 159 | CD2 | TYR | A | 27 | 38.525 | 38.316 | 70.938 | 1.00 | 7.94 | A |
| ATOM | 160 | CE2 | TYR | A | 27 | 39.379 | 37.704 | 70.022 | 1.00 | 8.81 | A |
| ATOM | 161 | CZ | TYR | A | 27 | 40.624 | 38.253 | 69.774 | 1.00 | 8.74 | A |
| ATOM | 162 | OH | TYR | A | 27 | 41.478 | 37.626 | 68.896 | 1.00 | 9.19 | A |
| ATOM | 163 | C | TYR | A | 27 | 37.044 | 41.873 | 71.216 | 1.00 | 8.37 | A |
| ATOM | 164 | O | TYR | A | 27 | 37.660 | 42.249 | 70.215 | 1.00 | 8.48 | A |
| ATOM | 165 | N | VAL | A | 28 | 35.727 | 41.718 | 71.235 | 1.00 | 5.90 | A |
| ATOM | 166 | CA | VAL | A | 28 | 34.909 | 41.911 | 70.047 | 1.00 | 8.51 | A |
| ATOM | 167 | CB | VAL | A | 28 | 33.598 | 41.105 | 70.177 | 1.00 | 5.91 | A |
| ATOM | 168 | CG1 | VAL | A | 28 | 32.639 | 41.456 | 69.048 | 1.00 | 7.76 | A |
| ATOM | 169 | CG2 | VAL | A | 28 | 33.912 | 39.616 | 70.162 | 1.00 | 7.37 | A |
| ATOM | 170 | C | VAL | A | 28 | 34.566 | 43.346 | 69.645 | 1.00 | 8.64 | A |
| ATOM | 171 | O | VAL | A | 28 | 34.576 | 43.666 | 68.454 | 1.00 | 9.91 | A |
| ATOM | 172 | N | THR | A | 29 | 34.263 | 44.204 | 70.616 | 1.00 | 8.56 | A |
| ATOM | 173 | CA | THR | A | 29 | 33.886 | 45.591 | 70.316 | 1.00 | 9.32 | A |
| ATOM | 174 | CB | THR | A | 29 | 32.444 | 45.881 | 70.767 | 1.00 | 10.71 | A |
| ATOM | 175 | OG1 | THR | A | 29 | 32.368 | 45.765 | 72.196 | 1.00 | 9.30 | A |
| ATOM | 176 | CG2 | THR | A | 29 | 31.471 | 44.901 | 70.134 | 1.00 | 13.06 | A |
| ATOM | 177 | C | THR | A | 29 | 34.765 | 46.642 | 70.990 | 1.00 | 10.10 | A |
| ATOM | 178 | O | THR | A | 29 | 34.574 | 47.844 | 70.782 | 1.00 | 9.56 | A |
| ATOM | 179 | N | ASN | A | 30 | 35.716 | 46.185 | 71.800 | 1.00 | 8.88 | A |
| ATOM | 180 | CA | ASN | A | 30 | 36.607 | 47.073 | 72.541 | 1.00 | 8.25 | A |
| ATOM | 181 | CB | ASN | A | 30 | 37.399 | 47.986 | 71.598 | 1.00 | 9.25 | A |
| ATOM | 182 | CG | ASN | A | 30 | 38.575 | 48.659 | 72.296 | 1.00 | 12.01 | A |
| ATOM | 183 | OD1 | ASN | A | 30 | 39.009 | 49.749 | 71.916 | 1.00 | 12.14 | A |
| ATOM | 184 | ND2 | ASN | A | 30 | 39.105 | 47.998 | 73.315 | 1.00 | 7.38 | A |
| ATOM | 185 | C | ASN | A | 30 | 35.806 | 47.939 | 73.516 | 1.00 | 8.22 | A |
| ATOM | 186 | O | ASN | A | 30 | 36.211 | 49.054 | 73.837 | 1.00 | 11.01 | A |
| ATOM | 187 | N | LYS | A | 31 | 34.664 | 47.432 | 73.974 | 1.00 | 9.33 | A |
| ATOM | 188 | CA | LYS | A | 31 | 33.827 | 48.168 | 74.923 | 1.00 | 9.57 | A |
| ATOM | 189 | CB | LYS | A | 31 | 32.391 | 48.280 | 74.403 | 1.00 | 10.37 | A |
| ATOM | 190 | CG | LYS | A | 31 | 32.241 | 49.198 | 73.197 | 1.00 | 10.72 | A |
| ATOM | 191 | CD | LYS | A | 31 | 30.793 | 49.302 | 72.763 | 1.00 | 11.43 | A |
| ATOM | 192 | CE | LYS | A | 31 | 30.643 | 50.305 | 71.636 | 1.00 | 13.28 | A |
| ATOM | 193 | NZ | LYS | A | 31 | 29.222 | 50.476 | 71.249 | 1.00 | 14.65 | A |
| ATOM | 194 | C | LYS | A | 31 | 33.817 | 47.483 | 76.290 | 1.00 | 10.19 | A |
| ATOM | 195 | O | LYS | A | 31 | 34.088 | 46.290 | 76.393 | 1.00 | 6.91 | A |
| ATOM | 196 | N | PHE | A | 32 | 33.496 | 48.249 | 77.331 | 1.00 | 11.11 | A |
| ATOM | 197 | CA | PHE | A | 32 | 33.446 | 47.727 | 78.695 | 1.00 | 9.90 | A |
| ATOM | 198 | CB | PHE | A | 32 | 34.793 | 47.949 | 79.393 | 1.00 | 10.23 | A |
| ATOM | 199 | CG | PHE | A | 32 | 34.800 | 47.550 | 80.845 | 1.00 | 8.36 | A |
| ATOM | 200 | CD1 | PHE | A | 32 | 34.821 | 46.207 | 81.210 | 1.00 | 8.88 | A |
| ATOM | 201 | CD2 | PHE | A | 32 | 34.769 | 48.518 | 81.848 | 1.00 | 10.04 | A |
| ATOM | 202 | CE1 | PHE | A | 32 | 34.812 | 45.830 | 82.558 | 1.00 | 8.21 | A |
| ATOM | 203 | CE2 | PHE | A | 32 | 34.759 | 48.152 | 83.200 | 1.00 | 11.57 | A |
| ATOM | 204 | CZ | PHE | A | 32 | 34.781 | 46.804 | 83.555 | 1.00 | 9.67 | A |
| ATOM | 205 | C | PHE | A | 32 | 32.349 | 48.407 | 79.512 | 1.00 | 12.49 | A |
| ATOM | 206 | O | PHE | A | 32 | 32.197 | 49.626 | 79.468 | 1.00 | 13.37 | A |
| ATOM | 207 | N | ASP | A | 33 | 31.585 | 47.615 | 80.257 | 1.00 | 13.61 | A |
| ATOM | 208 | CA | ASP | A | 33 | 30.524 | 48.156 | 81.100 | 1.00 | 15.70 | A |
| ATOM | 209 | CB | ASP | A | 33 | 29.169 | 47.553 | 80.723 | 1.00 | 19.01 | A |
| ATOM | 210 | CG | ASP | A | 33 | 28.787 | 47.832 | 79.282 | 1.00 | 23.57 | A |
| ATOM | 211 | OD1 | ASP | A | 33 | 29.393 | 47.227 | 78.373 | 1.00 | 29.17 | A |
| ATOM | 212 | OD2 | ASP | A | 33 | 27.883 | 48.662 | 79.055 | 1.00 | 27.35 | A |
| ATOM | 213 | C | ASP | A | 33 | 30.853 | 47.821 | 82.550 | 1.00 | 14.83 | A |
| ATOM | 214 | O | ASP | A | 33 | 31.173 | 46.679 | 82.869 | 1.00 | 12.67 | A |
| ATOM | 215 | N | THR | A | 34 | 30.778 | 48.817 | 83.426 | 1.00 | 14.91 | A |
| ATOM | 216 | CA | THR | A | 34 | 31.089 | 48.606 | 84.837 | 1.00 | 16.68 | A |
| ATOM | 217 | CB | THR | A | 34 | 31.408 | 49.923 | 85.545 | 1.00 | 17.04 | A |
| ATOM | 218 | OG1 | THR | A | 34 | 30.240 | 50.751 | 85.548 | 1.00 | 19.20 | A |
| ATOM | 219 | CG2 | THR | A | 34 | 32.538 | 50.638 | 84.842 | 1.00 | 18.49 | A |
| ATOM | 220 | C | THR | A | 34 | 29.957 | 47.947 | 85.602 | 1.00 | 17.04 | A |
| ATOM | 221 | O | THR | A | 34 | 30.195 | 47.266 | 86.597 | 1.00 | 17.92 | A |
| ATOM | 222 | N | GLN | A | 35 | 28.726 | 48.161 | 85.155 | 1.00 | 16.75 | A |
| ATOM | 223 | CA | GLN | A | 35 | 27.586 | 47.568 | 85.834 | 1.00 | 18.60 | A |
| ATOM | 224 | CB | GLN | A | 35 | 26.634 | 48.659 | 86.330 | 1.00 | 20.47 | A |
| ATOM | 225 | CG | GLN | A | 35 | 27.272 | 49.694 | 87.258 | 1.00 | 21.17 | A |
| ATOM | 226 | CD | GLN | A | 35 | 27.691 | 49.138 | 88.613 | 1.00 | 24.38 | A |
| ATOM | 227 | OE1 | GLN | A | 35 | 28.089 | 49.894 | 89.502 | 1.00 | 25.58 | A |
| ATOM | 228 | NE2 | GLN | A | 35 | 27.605 | 47.821 | 88.779 | 1.00 | 21.06 | A |
| ATOM | 229 | C | GLN | A | 35 | 26.846 | 46.601 | 84.923 | 1.00 | 17.95 | A |
| ATOM | 230 | O | GLN | A | 35 | 26.242 | 46.997 | 83.929 | 1.00 | 19.50 | A |
| ATOM | 231 | N | LEU | A | 36 | 26.910 | 45.322 | 85.275 | 1.00 | 17.18 | A |
| ATOM | 232 | CA | LEU | A | 36 | 26.255 | 44.270 | 84.512 | 1.00 | 16.20 | A |
| ATOM | 233 | CB | LEU | A | 36 | 27.287 | 43.433 | 83.748 | 1.00 | 17.22 | A |
| ATOM | 234 | CG | LEU | A | 36 | 28.074 | 44.042 | 82.587 | 1.00 | 18.60 | A |
| ATOM | 235 | CD1 | LEU | A | 36 | 27.114 | 44.508 | 81.493 | 1.00 | 18.57 | A |
| ATOM | 236 | CD2 | LEU | A | 36 | 28.913 | 45.188 | 83.094 | 1.00 | 21.87 | A |
| ATOM | 237 | C | LEU | A | 36 | 25.502 | 43.355 | 85.463 | 1.00 | 15.14 | A |
| ATOM | 238 | O | LEU | A | 36 | 25.497 | 43.563 | 86.679 | 1.00 | 14.61 | A |
| ATOM | 239 | N | PHE | A | 37 | 24.862 | 42.338 | 84.900 | 1.00 | 13.87 | A |
| ATOM | 240 | CA | PHE | A | 37 | 24.136 | 41.372 | 85.704 | 1.00 | 13.27 | A |
| ATOM | 241 | CB | PHE | A | 37 | 22.622 | 41.559 | 85.536 | 1.00 | 17.43 | A |
| ATOM | 242 | CG | PHE | A | 37 | 22.073 | 40.985 | 84.258 | 1.00 | 22.76 | A |
| ATOM | 243 | CD1 | PHE | A | 37 | 21.518 | 39.706 | 84.234 | 1.00 | 25.90 | A |
| ATOM | 244 | CD2 | PHE | A | 37 | 22.127 | 41.710 | 83.074 | 1.00 | 25.72 | A |
| ATOM | 245 | CE1 | PHE | A | 37 | 21.024 | 39.156 | 83.046 | 1.00 | 26.50 | A |
| ATOM | 246 | CE2 | PHE | A | 37 | 21.638 | 41.172 | 81.886 | 1.00 | 24.77 | A |
| ATOM | 247 | CZ | PHE | A | 37 | 21.086 | 39.894 | 81.872 | 1.00 | 28.92 | A |
| ATOM | 248 | C | PHE | A | 37 | 24.564 | 39.992 | 85.219 | 1.00 | 10.88 | A |
| ATOM | 249 | O | PHE | A | 37 | 24.994 | 39.832 | 84.074 | 1.00 | 10.13 | A |
| ATOM | 250 | N | HIS | A | 38 | 24.450 | 39.000 | 86.094 | 1.00 | 8.84 | A |
| ATOM | 251 | CA | HIS | A | 38 | 24.825 | 37.633 | 85.752 | 1.00 | 8.00 | A |
| ATOM | 252 | CB | HIS | A | 38 | 24.449 | 36.677 | 86.889 | 1.00 | 8.42 | A |
| ATOM | 253 | CG | HIS | A | 38 | 25.296 | 36.821 | 88.113 | 1.00 | 10.03 | A |
| ATOM | 254 | CD2 | HIS | A | 38 | 24.985 | 37.216 | 89.370 | 1.00 | 10.43 | A |
| ATOM | 255 | ND1 | HIS | A | 38 | 26.642 | 36.526 | 88.125 | 1.00 | 9.28 | A |
| ATOM | 256 | CE1 | HIS | A | 38 | 27.125 | 36.734 | 89.337 | 1.00 | 10.90 | A |
| ATOM | 257 | NE2 | HIS | A | 38 | 26.140 | 37.154 | 90.111 | 1.00 | 11.36 | A |
| ATOM | 258 | C | HIS | A | 38 | 24.136 | 37.149 | 84.483 | 1.00 | 8.41 | A |
| ATOM | 259 | O | HIS | A | 38 | 22.940 | 37.360 | 84.298 | 1.00 | 8.90 | A |
| ATOM | 260 | N | THR | A | 39 | 24.895 | 36.497 | 83.612 | 1.00 | 6.55 | A |
| ATOM | 261 | CA | THR | A | 39 | 24.335 | 35.939 | 82.385 | 1.00 | 7.97 | A |
| ATOM | 262 | CB | THR | A | 39 | 25.459 | 35.520 | 81.425 | 1.00 | 7.24 | A |
| ATOM | 263 | OG1 | THR | A | 39 | 26.192 | 36.683 | 81.036 | 1.00 | 5.54 | A |
| ATOM | 264 | CG2 | THR | A | 39 | 24.897 | 34.842 | 80.179 | 1.00 | 7.55 | A |
| ATOM | 265 | C | THR | A | 39 | 23.519 | 34.712 | 82.808 | 1.00 | 8.03 | A |
| ATOM | 266 | O | THR | A | 39 | 23.924 | 33.984 | 83.715 | 1.00 | 8.02 | A |
| ATOM | 267 | N | ILE | A | 40 | 22.371 | 34.496 | 82.169 | 1.00 | 7.72 | A |
| ATOM | 268 | CA | ILE | A | 40 | 21.503 | 33.365 | 82.515 | 1.00 | 9.99 | A |
| ATOM | 269 | CB | ILE | A | 40 | 20.093 | 33.874 | 82.911 | 1.00 | 11.99 | A |
| ATOM | 270 | CG2 | ILE | A | 40 | 19.179 | 32.715 | 83.274 | 1.00 | 18.33 | A |
| ATOM | 271 | CG1 | ILE | A | 40 | 20.207 | 34.810 | 84.112 | 1.00 | 16.11 | A |
| ATOM | 272 | CD1 | ILE | A | 40 | 18.940 | 35.589 | 84.379 | 1.00 | 19.11 | A |
| ATOM | 273 | C | ILE | A | 40 | 21.382 | 32.361 | 81.369 | 1.00 | 9.32 | A |
| ATOM | 274 | O | ILE | A | 40 | 20.542 | 31.454 | 81.393 | 1.00 | 8.85 | A |
| ATOM | 275 | N | GLY | A | 41 | 22.227 | 32.525 | 80.360 | 1.00 | 8.31 | A |
| ATOM | 276 | CA | GLY | A | 41 | 22.187 | 31.613 | 79.238 | 1.00 | 6.00 | A |
| ATOM | 277 | C | GLY | A | 41 | 23.565 | 31.438 | 78.639 | 1.00 | 7.09 | A |
| ATOM | 278 | O | GLY | A | 41 | 24.578 | 31.541 | 79.334 | 1.00 | 7.10 | A |
| ATOM | 279 | N | VAL | A | 42 | 23.594 | 31.174 | 77.339 | 1.00 | 6.98 | A |
| ATOM | 280 | CA | VAL | A | 42 | 24.841 | 30.990 | 76.611 | 1.00 | 6.92 | A |
| ATOM | 281 | CB | VAL | A | 42 | 25.007 | 29.528 | 76.151 | 1.00 | 4.81 | A |
| ATOM | 282 | CG1 | VAL | A | 42 | 26.221 | 29.392 | 75.225 | 1.00 | 7.05 | A |
| ATOM | 283 | CG2 | VAL | A | 42 | 25.165 | 28.627 | 77.367 | 1.00 | 6.21 | A |
| ATOM | 284 | C | VAL | A | 42 | 24.808 | 31.909 | 75.403 | 1.00 | 7.84 | A |
| ATOM | 285 | O | VAL | A | 42 | 23.762 | 32.102 | 74.778 | 1.00 | 7.04 | A |
| ATOM | 286 | N | GLU | A | 43 | 25.957 | 32.482 | 75.071 | 1.00 | 8.73 | A |
| ATOM | 287 | CA | GLU | A | 43 | 26.021 | 33.387 | 73.943 | 1.00 | 7.66 | A |
| ATOM | 288 | CB | GLU | A | 43 | 25.892 | 34.828 | 74.450 | 1.00 | 11.66 | A |
| ATOM | 289 | CG | GLU | A | 43 | 25.976 | 35.881 | 73.378 | 1.00 | 19.37 | A |
| ATOM | 290 | CD | GLU | A | 43 | 25.556 | 37.249 | 73.880 | 1.00 | 23.93 | A |
| ATOM | 291 | OE1 | GLU | A | 43 | 26.113 | 37.718 | 74.899 | 1.00 | 23.25 | A |
| ATOM | 292 | OE2 | GLU | A | 43 | 24.668 | 37.857 | 73.246 | 1.00 | 27.90 | A |
| ATOM | 293 | C | GLU | A | 43 | 27.323 | 33.187 | 73.189 | 1.00 | 7.28 | A |
| ATOM | 294 | O | GLU | A | 43 | 28.384 | 33.081 | 73.795 | 1.00 | 6.98 | A |
| ATOM | 295 | N | PHE | A | 44 | 27.239 | 33.093 | 71.868 | 1.00 | 6.23 | A |
| ATOM | 296 | CA | PHE | A | 44 | 28.445 | 32.922 | 71.080 | 1.00 | 7.46 | A |
| ATOM | 297 | CB | PHE | A | 44 | 28.743 | 31.431 | 70.830 | 1.00 | 6.39 | A |
| ATOM | 298 | CG | PHE | A | 44 | 27.688 | 30.699 | 70.034 | 1.00 | 5.36 | A |
| ATOM | 299 | CD1 | PHE | A | 44 | 26.589 | 30.125 | 70.667 | 1.00 | 5.62 | A |
| ATOM | 300 | CD2 | PHE | A | 44 | 27.826 | 30.540 | 68.655 | 1.00 | 6.00 | A |
| ATOM | 301 | CE1 | PHE | A | 44 | 25.640 | 29.396 | 69.945 | 1.00 | 4.28 | A |
| ATOM | 302 | CE2 | PHE | A | 44 | 26.886 | 29.814 | 67.921 | 1.00 | 5.90 | A |
| ATOM | 303 | CZ | PHE | A | 44 | 25.789 | 29.239 | 68.570 | 1.00 | 4.26 | A |
| ATOM | 304 | C | PHE | A | 44 | 28.393 | 33.693 | 69.771 | 1.00 | 7.79 | A |
| ATOM | 305 | O | PHE | A | 44 | 27.340 | 34.193 | 69.366 | 1.00 | 6.57 | A |
| ATOM | 306 | N | LEU | A | 45 | 29.552 | 33.823 | 69.135 | 1.00 | 7.42 | A |
| ATOM | 307 | CA | LEU | A | 45 | 29.661 | 34.545 | 67.876 | 1.00 | 8.13 | A |
| ATOM | 308 | CB | LEU | A | 45 | 29.570 | 36.054 | 68.138 | 1.00 | 8.36 | A |
| ATOM | 309 | CG | LEU | A | 45 | 30.407 | 36.590 | 69.307 | 1.00 | 10.28 | A |
| ATOM | 310 | CD1 | LEU | A | 45 | 31.885 | 36.476 | 68.986 | 1.00 | 12.35 | A |
| ATOM | 311 | CD2 | LEU | A | 45 | 30.040 | 38.035 | 69.576 | 1.00 | 15.28 | A |
| ATOM | 312 | C | LEU | A | 45 | 30.982 | 34.192 | 67.200 | 1.00 | 6.72 | A |
| ATOM | 313 | O | LEU | A | 45 | 31.818 | 33.516 | 67.795 | 1.00 | 4.96 | A |
| ATOM | 314 | N | ASN | A | 46 | 31.161 | 34.632 | 65.958 | 1.00 | 7.24 | A |
| ATOM | 315 | CA | ASN | A | 46 | 32.396 | 34.350 | 65.228 | 1.00 | 6.86 | A |
| ATOM | 316 | CB | ASN | A | 46 | 32.109 | 33.815 | 63.815 | 1.00 | 7.39 | A |
| ATOM | 317 | CG | ASN | A | 46 | 31.575 | 32.395 | 63.801 | 1.00 | 5.99 | A |
| ATOM | 318 | OD1 | ASN | A | 46 | 31.641 | 31.671 | 64.791 | 1.00 | 5.95 | A |
| ATOM | 319 | ND2 | ASN | A | 46 | 31.054 | 31.985 | 62.650 | 1.00 | 6.59 | A |
| ATOM | 320 | C | ASN | A | 46 | 33.225 | 35.620 | 65.073 | 1.00 | 7.51 | A |
| ATOM | 321 | O | ASN | A | 46 | 32.679 | 36.698 | 64.844 | 1.00 | 7.51 | A |
| ATOM | 322 | N | LYS | A | 47 | 34.543 | 35.489 | 65.212 | 1.00 | 6.91 | A |
| ATOM | 323 | CA | LYS | A | 47 | 35.448 | 36.621 | 65.024 | 1.00 | 7.75 | A |
| ATOM | 324 | CB | LYS | A | 47 | 35.843 | 37.266 | 66.359 | 1.00 | 8.75 | A |
| ATOM | 325 | CG | LYS | A | 47 | 36.829 | 38.435 | 66.198 | 1.00 | 8.17 | A |
| ATOM | 326 | CD | LYS | A | 47 | 37.026 | 39.196 | 67.506 | 1.00 | 9.00 | A |
| ATOM | 327 | CE | LYS | A | 47 | 38.191 | 40.183 | 67.423 | 1.00 | 10.90 | A |
| ATOM | 328 | NZ | LYS | A | 47 | 38.000 | 41.240 | 66.384 | 1.00 | 11.38 | A |
| ATOM | 329 | C | LYS | A | 47 | 36.687 | 36.104 | 64.307 | 1.00 | 8.30 | A |
| ATOM | 330 | O | LYS | A | 47 | 37.323 | 35.148 | 64.755 | 1.00 | 7.55 | A |
| ATOM | 331 | N | ASP | A | 48 | 37.022 | 36.730 | 63.183 | 1.00 | 8.48 | A |
| ATOM | 332 | CA | ASP | A | 48 | 38.180 | 36.302 | 62.410 | 1.00 | 8.90 | A |
| ATOM | 333 | CB | ASP | A | 48 | 38.014 | 36.677 | 60.932 | 1.00 | 11.08 | A |
| ATOM | 334 | CG | ASP | A | 48 | 36.790 | 36.050 | 60.297 | 1.00 | 14.69 | A |
| ATOM | 335 | OD1 | ASP | A | 48 | 36.346 | 34.976 | 60.756 | 1.00 | 13.69 | A |
| ATOM | 336 | OD2 | ASP | A | 48 | 36.279 | 36.630 | 59.316 | 1.00 | 18.84 | A |
| ATOM | 337 | C | ASP | A | 48 | 39.497 | 36.889 | 62.905 | 1.00 | 8.15 | A |
| ATOM | 338 | O | ASP | A | 48 | 39.543 | 37.991 | 63.453 | 1.00 | 8.51 | A |
| ATOM | 339 | N | LEU | A | 49 | 40.569 | 36.131 | 62.707 | 1.00 | 6.89 | A |
| ATOM | 340 | CA | LEU | A | 49 | 41.901 | 36.581 | 63.078 | 1.00 | 7.26 | A |
| ATOM | 341 | CB | LEU | A | 49 | 42.137 | 36.473 | 64.591 | 1.00 | 7.57 | A |
| ATOM | 342 | CG | LEU | A | 49 | 42.421 | 35.103 | 65.210 | 1.00 | 8.18 | A |
| ATOM | 343 | CD1 | LEU | A | 49 | 42.987 | 35.294 | 66.619 | 1.00 | 8.46 | A |
| ATOM | 344 | CD2 | LEU | A | 49 | 41.146 | 34.266 | 65.240 | 1.00 | 8.38 | A |
| ATOM | 345 | C | LEU | A | 49 | 42.903 | 35.717 | 62.343 | 1.00 | 7.32 | A |
| ATOM | 346 | O | LEU | A | 49 | 42.549 | 34.662 | 61.814 | 1.00 | 9.38 | A |
| ATOM | 347 | N | GLU | A | 50 | 44.151 | 36.170 | 62.299 | 1.00 | 8.09 | A |
| ATOM | 348 | CA | GLU | A | 50 | 45.207 | 35.421 | 61.633 | 1.00 | 8.78 | A |
| ATOM | 349 | CB | GLU | A | 50 | 45.707 | 36.165 | 60.388 | 1.00 | 9.51 | A |
| ATOM | 350 | CG | GLU | A | 50 | 46.786 | 35.380 | 59.649 | 1.00 | 11.60 | A |
| ATOM | 351 | CD | GLU | A | 50 | 47.359 | 36.097 | 58.440 | 1.00 | 14.08 | A |
| ATOM | 352 | OE1 | GLU | A | 50 | 47.079 | 37.299 | 58.251 | 1.00 | 15.59 | A |
| ATOM | 353 | OE2 | GLU | A | 50 | 48.109 | 35.447 | 57.683 | 1.00 | 13.72 | A |
| ATOM | 354 | C | GLU | A | 50 | 46.370 | 35.201 | 62.593 | 1.00 | 8.34 | A |
| ATOM | 355 | O | GLU | A | 50 | 46.838 | 36.134 | 63.241 | 1.00 | 8.19 | A |
| ATOM | 356 | N | VAL | A | 51 | 46.826 | 33.958 | 62.682 | 1.00 | 8.80 | A |
| ATOM | 357 | CA | VAL | A | 51 | 47.938 | 33.598 | 63.554 | 1.00 | 8.28 | A |
| ATOM | 358 | CB | VAL | A | 51 | 47.441 | 32.968 | 64.862 | 1.00 | 8.80 | A |
| ATOM | 359 | CG1 | VAL | A | 51 | 46.619 | 33.970 | 65.661 | 1.00 | 7.51 | A |
| ATOM | 360 | CG2 | VAL | A | 51 | 46.618 | 31.755 | 64.545 | 1.00 | 13.00 | A |
| ATOM | 361 | C | VAL | A | 51 | 48.800 | 32.576 | 62.824 | 1.00 | 7.24 | A |
| ATOM | 362 | O | VAL | A | 51 | 48.282 | 31.610 | 62.261 | 1.00 | 7.96 | A |
| ATOM | 363 | N | ASP | A | 52 | 50.114 | 32.781 | 62.852 | 1.00 | 8.14 | A |
| ATOM | 364 | CA | ASP | A | 52 | 51.047 | 31.880 | 62.179 | 1.00 | 9.08 | A |
| ATOM | 365 | CB | ASP | A | 52 | 51.090 | 30.514 | 62.879 | 1.00 | 9.41 | A |
| ATOM | 366 | CG | ASP | A | 52 | 51.859 | 30.541 | 64.189 | 1.00 | 10.22 | A |
| ATOM | 367 | OD1 | ASP | A | 52 | 52.483 | 31.574 | 64.511 | 1.00 | 10.39 | A |
| ATOM | 368 | OD2 | ASP | A | 52 | 51.846 | 29.511 | 64.898 | 1.00 | 13.53 | A |
| ATOM | 369 | C | ASP | A | 52 | 50.685 | 31.669 | 60.707 | 1.00 | 8.35 | A |
| ATOM | 370 | O | ASP | A | 52 | 50.966 | 30.615 | 60.140 | 1.00 | 11.29 | A |
| ATOM | 371 | N | GLY | A | 53 | 50.055 | 32.660 | 60.089 | 1.00 | 7.50 | A |
| ATOM | 372 | CA | GLY | A | 53 | 49.695 | 32.518 | 58.690 | 1.00 | 9.04 | A |
| ATOM | 373 | C | GLY | A | 53 | 48.427 | 31.721 | 58.458 | 1.00 | 9.48 | A |
| ATOM | 374 | O | GLY | A | 53 | 48.071 | 31.420 | 57.311 | 1.00 | 9.43 | A |
| ATOM | 375 | N | HIS | A | 54 | 47.746 | 31.360 | 59.540 | 1.00 | 8.31 | A |
| ATOM | 376 | CA | HIS | A | 54 | 46.494 | 30.625 | 59.415 | 1.00 | 8.13 | A |
| ATOM | 377 | CB | HIS | A | 54 | 46.383 | 29.527 | 60.479 | 1.00 | 8.93 | A |
| ATOM | 378 | CG | HIS | A | 54 | 47.255 | 28.338 | 60.227 | 1.00 | 9.69 | A |
| ATOM | 379 | CD2 | HIS | A | 54 | 46.958 | 27.102 | 59.760 | 1.00 | 10.30 | A |
| ATOM | 380 | ND1 | HIS | A | 54 | 48.612 | 28.338 | 60.473 | 1.00 | 11.41 | A |
| ATOM | 381 | CE1 | HIS | A | 54 | 49.112 | 27.153 | 60.171 | 1.00 | 9.94 | A |
| ATOM | 382 | NE2 | HIS | A | 54 | 48.130 | 26.385 | 59.735 | 1.00 | 13.73 | A |
| ATOM | 383 | C | HIS | A | 54 | 45.324 | 31.580 | 59.591 | 1.00 | 9.01 | A |
| ATOM | 384 | O | HIS | A | 54 | 45.245 | 32.286 | 60.597 | 1.00 | 9.42 | A |
| ATOM | 385 | N | PHE | A | 55 | 44.425 | 31.618 | 58.611 | 1.00 | 9.45 | A |
| ATOM | 386 | CA | PHE | A | 55 | 43.250 | 32.473 | 58.711 | 1.00 | 10.34 | A |
| ATOM | 387 | CB | PHE | A | 55 | 42.789 | 32.931 | 57.325 | 1.00 | 12.55 | A |
| ATOM | 388 | CG | PHE | A | 55 | 43.741 | 33.890 | 56.673 | 1.00 | 14.47 | A |
| ATOM | 389 | CD1 | PHE | A | 55 | 44.838 | 33.424 | 55.958 | 1.00 | 16.74 | A |
| ATOM | 390 | CD2 | PHE | A | 55 | 43.571 | 35.262 | 56.821 | 1.00 | 17.13 | A |
| ATOM | 391 | CE1 | PHE | A | 55 | 45.756 | 34.313 | 55.400 | 1.00 | 16.83 | A |
| ATOM | 392 | CE2 | PHE | A | 55 | 44.483 | 36.160 | 56.269 | 1.00 | 16.96 | A |
| ATOM | 393 | CZ | PHE | A | 55 | 45.577 | 35.683 | 55.558 | 1.00 | 17.92 | A |
| ATOM | 394 | C | PHE | A | 55 | 42.182 | 31.646 | 59.407 | 1.00 | 10.74 | A |
| ATOM | 395 | O | PHE | A | 55 | 41.665 | 30.671 | 58.862 | 1.00 | 12.26 | A |
| ATOM | 396 | N | VAL | A | 56 | 41.865 | 32.043 | 60.630 | 1.00 | 8.99 | A |
| ATOM | 397 | CA | VAL | A | 56 | 40.912 | 31.310 | 61.441 | 1.00 | 10.74 | A |
| ATOM | 398 | CB | VAL | A | 56 | 41.600 | 30.835 | 62.748 | 1.00 | 14.68 | A |
| ATOM | 399 | CG1 | VAL | A | 56 | 40.620 | 30.077 | 63.627 | 1.00 | 18.84 | A |
| ATOM | 400 | CG2 | VAL | A | 56 | 42.802 | 29.971 | 62.411 | 1.00 | 16.44 | A |
| ATOM | 401 | C | VAL | A | 56 | 39.689 | 32.125 | 61.819 | 1.00 | 10.07 | A |
| ATOM | 402 | O | VAL | A | 56 | 39.739 | 33.350 | 61.877 | 1.00 | 7.41 | A |
| ATOM | 403 | N | THR | A | 57 | 38.582 | 31.427 | 62.036 | 1.00 | 6.30 | A |
| ATOM | 404 | CA | THR | A | 57 | 37.361 | 32.061 | 62.490 | 1.00 | 6.65 | A |
| ATOM | 405 | CB | THR | A | 57 | 36.137 | 31.670 | 61.634 | 1.00 | 5.91 | A |
| ATOM | 406 | OG1 | THR | A | 57 | 36.300 | 32.180 | 60.303 | 1.00 | 7.32 | A |
| ATOM | 407 | CG2 | THR | A | 57 | 34.862 | 32.252 | 62.237 | 1.00 | 8.86 | A |
| ATOM | 408 | C | THR | A | 57 | 37.203 | 31.476 | 63.886 | 1.00 | 5.83 | A |
| ATOM | 409 | O | THR | A | 57 | 36.957 | 30.283 | 64.037 | 1.00 | 8.06 | A |
| ATOM | 410 | N | MET | A | 58 | 37.392 | 32.305 | 64.906 | 1.00 | 7.18 | A |
| ATOM | 411 | CA | MET | A | 58 | 37.256 | 31.844 | 66.279 | 1.00 | 6.31 | A |
| ATOM | 412 | CB | MET | A | 58 | 38.189 | 32.622 | 67.213 | 1.00 | 8.44 | A |
| ATOM | 413 | CG | MET | A | 58 | 38.082 | 32.187 | 68.678 | 1.00 | 10.17 | A |
| ATOM | 414 | SD | MET | A | 58 | 38.991 | 33.278 | 69.790 | 1.00 | 12.03 | A |
| ATOM | 415 | CE | MET | A | 58 | 40.633 | 32.927 | 69.290 | 1.00 | 13.90 | A |
| ATOM | 416 | C | MET | A | 58 | 35.821 | 32.041 | 66.739 | 1.00 | 7.83 | A |
| ATOM | 417 | O | MET | A | 58 | 35.298 | 33.154 | 66.703 | 1.00 | 7.74 | A |
| ATOM | 418 | N | GLN | A | 59 | 35.181 | 30.954 | 67.157 | 1.00 | 6.26 | A |
| ATOM | 419 | CA | GLN | A | 59 | 33.818 | 31.033 | 67.647 | 1.00 | 6.62 | A |
| ATOM | 420 | CB | GLN | A | 59 | 33.007 | 29.836 | 67.165 | 1.00 | 7.00 | A |
| ATOM | 421 | CG | GLN | A | 59 | 31.532 | 29.935 | 67.491 | 1.00 | 6.31 | A |
| ATOM | 422 | CD | GLN | A | 59 | 30.714 | 28.914 | 66.731 | 1.00 | 5.47 | A |
| ATOM | 423 | OE1 | GLN | A | 59 | 30.332 | 29.133 | 65.570 | 1.00 | 9.16 | A |
| ATOM | 424 | NE2 | GLN | A | 59 | 30.455 | 27.785 | 67.366 | 1.00 | 4.49 | A |
| ATOM | 425 | C | GLN | A | 59 | 33.927 | 31.044 | 69.163 | 1.00 | 5.91 | A |
| ATOM | 426 | O | GLN | A | 59 | 34.268 | 30.038 | 69.790 | 1.00 | 5.92 | A |
| ATOM | 427 | N | ILE | A | 60 | 33.653 | 32.204 | 69.740 | 1.00 | 6.63 | A |
| ATOM | 428 | CA | ILE | A | 60 | 33.749 | 32.400 | 71.176 | 1.00 | 7.03 | A |
| ATOM | 429 | CB | ILE | A | 60 | 34.166 | 33.847 | 71.491 | 1.00 | 8.49 | A |
| ATOM | 430 | CG2 | ILE | A | 60 | 34.463 | 33.998 | 72.977 | 1.00 | 9.60 | A |
| ATOM | 431 | CG1 | ILE | A | 60 | 35.400 | 34.217 | 70.664 | 1.00 | 9.66 | A |
| ATOM | 432 | CD1 | ILE | A | 60 | 35.790 | 35.678 | 70.777 | 1.00 | 8.16 | A |
| ATOM | 433 | C | ILE | A | 60 | 32.433 | 32.121 | 71.871 | 1.00 | 6.63 | A |
| ATOM | 434 | O | ILE | A | 60 | 31.416 | 32.749 | 71.564 | 1.00 | 8.29 | A |
| ATOM | 435 | N | TRP | A | 61 | 32.466 | 31.184 | 72.813 | 1.00 | 5.15 | A |
| ATOM | 436 | CA | TRP | A | 61 | 31.288 | 30.812 | 73.588 | 1.00 | 5.48 | A |
| ATOM | 437 | CB | TRP | A | 61 | 31.131 | 29.290 | 73.620 | 1.00 | 3.75 | A |
| ATOM | 438 | CG | TRP | A | 61 | 30.647 | 28.685 | 72.339 | 1.00 | 4.93 | A |
| ATOM | 439 | CD2 | TRP | A | 61 | 29.440 | 27.933 | 72.157 | 1.00 | 4.76 | A |
| ATOM | 440 | CE2 | TRP | A | 61 | 29.375 | 27.570 | 70.794 | 1.00 | 6.00 | A |
| ATOM | 441 | CE3 | TRP | A | 61 | 28.405 | 27.533 | 73.015 | 1.00 | 7.98 | A |
| ATOM | 442 | CD1 | TRP | A | 61 | 31.250 | 28.744 | 71.121 | 1.00 | 5.36 | A |
| ATOM | 443 | NE1 | TRP | A | 61 | 30.490 | 28.076 | 70.180 | 1.00 | 3.63 | A |
| ATOM | 444 | CZ2 | TRP | A | 61 | 28.313 | 26.824 | 70.264 | 1.00 | 5.06 | A |
| ATOM | 445 | CZ3 | TRP | A | 61 | 27.344 | 26.787 | 72.484 | 1.00 | 6.13 | A |
| ATOM | 446 | CH2 | TRP | A | 61 | 27.311 | 26.444 | 71.122 | 1.00 | 6.59 | A |
| ATOM | 447 | C | TRP | A | 61 | 31.383 | 31.324 | 75.026 | 1.00 | 6.51 | A |
| ATOM | 448 | O | TRP | A | 61 | 32.256 | 30.903 | 75.785 | 1.00 | 7.41 | A |
| ATOM | 449 | N | ASP | A | 62 | 30.479 | 32.228 | 75.389 | 1.00 | 6.09 | A |
| ATOM | 450 | CA | ASP | A | 62 | 30.422 | 32.786 | 76.739 | 1.00 | 5.83 | A |
| ATOM | 451 | CB | ASP | A | 62 | 30.134 | 34.289 | 76.665 | 1.00 | 6.61 | A |
| ATOM | 452 | CG | ASP | A | 62 | 29.792 | 34.903 | 78.020 | 1.00 | 6.69 | A |
| ATOM | 453 | OD1 | ASP | A | 62 | 30.236 | 34.370 | 79.061 | 1.00 | 6.95 | A |
| ATOM | 454 | OD2 | ASP | A | 62 | 29.084 | 35.937 | 78.034 | 1.00 | 8.01 | A |
| ATOM | 455 | C | ASP | A | 62 | 29.288 | 32.043 | 77.435 | 1.00 | 7.57 | A |
| ATOM | 456 | O | ASP | A | 62 | 28.168 | 32.007 | 76.935 | 1.00 | 7.66 | A |
| ATOM | 457 | N | THR | A | 63 | 29.581 | 31.432 | 78.577 | 1.00 | 8.39 | A |
| ATOM | 458 | CA | THR | A | 63 | 28.563 | 30.677 | 79.302 | 1.00 | 9.19 | A |
| ATOM | 459 | CB | THR | A | 63 | 29.006 | 29.219 | 79.523 | 1.00 | 9.87 | A |
| ATOM | 460 | OG1 | THR | A | 63 | 30.168 | 29.207 | 80.363 | 1.00 | 9.72 | A |
| ATOM | 461 | CG2 | THR | A | 63 | 29.336 | 28.545 | 78.199 | 1.00 | 11.03 | A |
| ATOM | 462 | C | THR | A | 63 | 28.287 | 31.265 | 80.675 | 1.00 | 7.76 | A |
| ATOM | 463 | O | THR | A | 63 | 29.168 | 31.864 | 81.289 | 1.00 | 8.83 | A |
| ATOM | 464 | N | ALA | A | 64 | 27.059 | 31.089 | 81.153 | 1.00 | 7.97 | A |
| ATOM | 465 | CA | ALA | A | 64 | 26.696 | 31.566 | 82.483 | 1.00 | 7.49 | A |
| ATOM | 466 | CB | ALA | A | 64 | 25.223 | 31.301 | 82.747 | 1.00 | 6.65 | A |
| ATOM | 467 | C | ALA | A | 64 | 27.562 | 30.754 | 83.446 | 1.00 | 6.51 | A |
| ATOM | 468 | O | ALA | A | 64 | 27.566 | 29.525 | 83.384 | 1.00 | 9.35 | A |
| ATOM | 469 | N | GLY | A | 65 | 28.293 | 31.437 | 84.325 | 1.00 | 6.53 | A |
| ATOM | 470 | CA | GLY | A | 65 | 29.177 | 30.745 | 85.249 | 1.00 | 5.87 | A |
| ATOM | 471 | C | GLY | A | 65 | 28.590 | 30.201 | 86.541 | 1.00 | 6.29 | A |
| ATOM | 472 | O | GLY | A | 65 | 29.234 | 29.389 | 87.219 | 1.00 | 7.83 | A |
| ATOM | 473 | N | GLN | A | 66 | 27.382 | 30.625 | 86.901 | 1.00 | 5.69 | A |
| ATOM | 474 | CA | GLN | A | 66 | 26.781 | 30.132 | 88.139 | 1.00 | 7.18 | A |
| ATOM | 475 | CB | GLN | A | 66 | 25.541 | 30.952 | 88.497 | 1.00 | 7.37 | A |
| ATOM | 476 | CG | GLN | A | 66 | 25.889 | 32.335 | 89.016 | 1.00 | 9.92 | A |
| ATOM | 477 | CD | GLN | A | 66 | 24.662 | 33.189 | 89.262 | 1.00 | 10.10 | A |
| ATOM | 478 | OE1 | GLN | A | 66 | 24.027 | 33.682 | 88.321 | 1.00 | 11.79 | A |
| ATOM | 479 | NE2 | GLN | A | 66 | 24.310 | 33.358 | 90.530 | 1.00 | 9.58 | A |
| ATOM | 480 | C | GLN | A | 66 | 26.449 | 28.645 | 88.070 | 1.00 | 6.14 | A |
| ATOM | 481 | O | GLN | A | 66 | 26.121 | 28.116 | 87.012 | 1.00 | 6.06 | A |
| ATOM | 482 | N | GLU | A | 67 | 26.532 | 27.980 | 89.219 | 1.00 | 8.00 | A |
| ATOM | 483 | CA | GLU | A | 67 | 26.296 | 26.544 | 89.305 | 1.00 | 7.42 | A |
| ATOM | 484 | CB | GLU | A | 67 | 26.464 | 26.075 | 90.755 | 1.00 | 11.32 | A |
| ATOM | 485 | CG | GLU | A | 67 | 27.850 | 26.317 | 91.330 | 0.00 | 11.52 | A |
| ATOM | 486 | CD | GLU | A | 67 | 27.970 | 25.862 | 92.771 | 0.00 | 12.39 | A |
| ATOM | 487 | OE1 | GLU | A | 67 | 27.755 | 24.660 | 93.035 | 0.00 | 12.65 | A |
| ATOM | 488 | OE2 | GLU | A | 67 | 28.280 | 26.705 | 93.639 | 0.00 | 12.65 | A |
| ATOM | 489 | C | GLU | A | 67 | 24.961 | 26.042 | 88.770 | 1.00 | 7.67 | A |
| ATOM | 490 | O | GLU | A | 67 | 24.905 | 24.981 | 88.163 | 1.00 | 7.31 | A |
| ATOM | 491 | N | ARG | A | 68 | 23.884 | 26.792 | 88.972 | 1.00 | 7.88 | A |
| ATOM | 492 | CA | ARG | A | 68 | 22.590 | 26.312 | 88.493 | 1.00 | 9.18 | A |
| ATOM | 493 | CB | ARG | A | 68 | 21.449 | 27.150 | 89.078 | 1.00 | 10.38 | A |
| ATOM | 494 | CG | ARG | A | 68 | 21.404 | 28.588 | 88.630 | 1.00 | 12.77 | A |
| ATOM | 495 | CD | ARG | A | 68 | 20.151 | 29.251 | 89.194 | 1.00 | 15.36 | A |
| ATOM | 496 | NE | ARG | A | 68 | 19.956 | 30.607 | 88.697 | 1.00 | 16.31 | A |
| ATOM | 497 | CZ | ARG | A | 68 | 20.716 | 31.645 | 89.028 | 1.00 | 16.59 | A |
| ATOM | 498 | NH1 | ARG | A | 68 | 21.735 | 31.488 | 89.864 | 1.00 | 16.11 | A |
| ATOM | 499 | NH2 | ARG | A | 68 | 20.450 | 32.842 | 88.523 | 1.00 | 17.25 | A |
| ATOM | 500 | C | ARG | A | 68 | 22.470 | 26.253 | 86.972 | 1.00 | 9.10 | A |
| ATOM | 501 | O | ARG | A | 68 | 21.478 | 25.743 | 86.442 | 1.00 | 9.38 | A |
| ATOM | 502 | N | PHE | A | 69 | 23.472 | 26.768 | 86.265 | 1.00 | 7.20 | A |
| ATOM | 503 | CA | PHE | A | 69 | 23.442 | 26.734 | 84.811 | 1.00 | 6.75 | A |
| ATOM | 504 | CB | PHE | A | 69 | 23.848 | 28.095 | 84.238 | 1.00 | 6.08 | A |
| ATOM | 505 | CG | PHE | A | 69 | 22.903 | 29.193 | 84.613 | 1.00 | 7.44 | A |
| ATOM | 506 | CD1 | PHE | A | 69 | 23.283 | 30.186 | 85.508 | 1.00 | 7.13 | A |
| ATOM | 507 | CD2 | PHE | A | 69 | 21.601 | 29.190 | 84.130 | 1.00 | 9.53 | A |
| ATOM | 508 | CE1 | PHE | A | 69 | 22.378 | 31.158 | 85.923 | 1.00 | 8.69 | A |
| ATOM | 509 | CE2 | PHE | A | 69 | 20.689 | 30.154 | 84.538 | 1.00 | 9.63 | A |
| ATOM | 510 | CZ | PHE | A | 69 | 21.076 | 31.142 | 85.439 | 1.00 | 8.24 | A |
| ATOM | 511 | C | PHE | A | 69 | 24.310 | 25.627 | 84.220 | 1.00 | 7.27 | A |
| ATOM | 512 | O | PHE | A | 69 | 24.520 | 25.572 | 83.005 | 1.00 | 7.36 | A |
| ATOM | 513 | N | ARG | A | 70 | 24.794 | 24.727 | 85.070 | 1.00 | 6.98 | A |
| ATOM | 514 | CA | ARG | A | 70 | 25.624 | 23.634 | 84.583 | 1.00 | 5.91 | A |
| ATOM | 515 | CB | ARG | A | 70 | 26.137 | 22.784 | 85.750 | 1.00 | 7.51 | A |
| ATOM | 516 | CG | ARG | A | 70 | 27.052 | 21.652 | 85.302 | 1.00 | 7.55 | A |
| ATOM | 517 | CD | ARG | A | 70 | 27.425 | 20.754 | 86.469 | 1.00 | 8.22 | A |
| ATOM | 518 | NE | ARG | A | 70 | 28.282 | 21.439 | 87.434 | 1.00 | 10.35 | A |
| ATOM | 519 | CZ | ARG | A | 70 | 28.532 | 20.977 | 88.654 | 1.00 | 13.82 | A |
| ATOM | 520 | NH1 | ARG | A | 70 | 27.983 | 19.835 | 89.047 | 1.00 | 13.09 | A |
| ATOM | 521 | NH2 | ARG | A | 70 | 29.333 | 21.650 | 89.476 | 1.00 | 14.67 | A |
| ATOM | 522 | C | ARG | A | 70 | 24.893 | 22.737 | 83.583 | 1.00 | 6.82 | A |
| ATOM | 523 | O | ARG | A | 70 | 25.444 | 22.395 | 82.536 | 1.00 | 6.19 | A |
| ATOM | 524 | N | SER | A | 71 | 23.654 | 22.356 | 83.892 | 1.00 | 8.11 | A |
| ATOM | 525 | CA | SER | A | 71 | 22.914 | 21.486 | 82.986 | 1.00 | 8.45 | A |
| ATOM | 526 | CB | SER | A | 71 | 21.575 | 21.050 | 83.608 | 1.00 | 11.15 | A |
| ATOM | 527 | OG | SER | A | 71 | 20.711 | 22.149 | 83.835 | 1.00 | 17.11 | A |
| ATOM | 528 | C | SER | A | 71 | 22.681 | 22.150 | 81.632 | 1.00 | 7.62 | A |
| ATOM | 529 | O | SER | A | 71 | 22.580 | 21.469 | 80.617 | 1.00 | 6.74 | A |
| ATOM | 530 | N | LEU | A | 72 | 22.609 | 23.477 | 81.617 | 1.00 | 6.32 | A |
| ATOM | 531 | CA | LEU | A | 72 | 22.406 | 24.203 | 80.369 | 1.00 | 6.96 | A |
| ATOM | 532 | CB | LEU | A | 72 | 22.066 | 25.676 | 80.647 | 1.00 | 4.94 | A |
| ATOM | 533 | CG | LEU | A | 72 | 22.051 | 26.588 | 79.411 | 1.00 | 6.62 | A |
| ATOM | 534 | CD1 | LEU | A | 72 | 20.841 | 26.258 | 78.549 | 1.00 | 9.17 | A |
| ATOM | 535 | CD2 | LEU | A | 72 | 22.000 | 28.052 | 79.836 | 1.00 | 8.56 | A |
| ATOM | 536 | C | LEU | A | 72 | 23.637 | 24.165 | 79.471 | 1.00 | 7.32 | A |
| ATOM | 537 | O | LEU | A | 72 | 23.551 | 23.839 | 78.287 | 1.00 | 7.01 | A |
| ATOM | 538 | N | ARG | A | 73 | 24.788 | 24.501 | 80.042 | 1.00 | 7.43 | A |
| ATOM | 539 | CA | ARG | A | 73 | 26.023 | 24.585 | 79.272 | 1.00 | 6.40 | A |
| ATOM | 540 | CB | ARG | A | 73 | 26.953 | 25.618 | 79.934 | 1.00 | 6.38 | A |
| ATOM | 541 | CG | ARG | A | 73 | 27.330 | 25.255 | 81.360 | 1.00 | 6.74 | A |
| ATOM | 542 | CD | ARG | A | 73 | 28.293 | 26.235 | 82.043 | 1.00 | 6.69 | A |
| ATOM | 543 | NE | ARG | A | 73 | 28.795 | 25.598 | 83.259 | 1.00 | 5.69 | A |
| ATOM | 544 | CZ | ARG | A | 73 | 28.476 | 25.946 | 84.501 | 1.00 | 6.76 | A |
| ATOM | 545 | NH1 | ARG | A | 73 | 27.656 | 26.960 | 84.738 | 1.00 | 3.17 | A |
| ATOM | 546 | NH2 | ARG | A | 73 | 28.945 | 25.230 | 85.515 | 1.00 | 6.50 | A |
| ATOM | 547 | C | ARG | A | 73 | 26.820 | 23.307 | 78.987 | 1.00 | 7.08 | A |
| ATOM | 548 | O | ARG | A | 73 | 27.450 | 23.203 | 77.927 | 1.00 | 7.04 | A |
| ATOM | 549 | N | THR | A | 74 | 26.797 | 22.333 | 79.895 | 1.00 | 8.63 | A |
| ATOM | 550 | CA | THR | A | 74 | 27.605 | 21.130 | 79.687 | 1.00 | 6.86 | A |
| ATOM | 551 | CB | THR | A | 74 | 27.452 | 20.117 | 80.857 | 1.00 | 8.59 | A |
| ATOM | 552 | OG1 | THR | A | 74 | 26.068 | 19.861 | 81.113 | 1.00 | 12.06 | A |
| ATOM | 553 | CG2 | THR | A | 74 | 28.112 | 20.667 | 82.117 | 1.00 | 9.00 | A |
| ATOM | 554 | C | THR | A | 74 | 27.434 | 20.408 | 78.353 | 1.00 | 8.68 | A |
| ATOM | 555 | O | THR | A | 74 | 28.412 | 19.939 | 77.773 | 1.00 | 9.70 | A |
| ATOM | 556 | N | PRO | A | 75 | 26.201 | 20.300 | 77.841 | 1.00 | 10.00 | A |
| ATOM | 557 | CD | PRO | A | 75 | 24.879 | 20.632 | 78.399 | 1.00 | 10.89 | A |
| ATOM | 558 | CA | PRO | A | 75 | 26.075 | 19.609 | 76.557 | 1.00 | 9.92 | A |
| ATOM | 559 | CB | PRO | A | 75 | 24.559 | 19.544 | 76.339 | 1.00 | 11.11 | A |
| ATOM | 560 | CG | PRO | A | 75 | 24.025 | 20.672 | 77.165 | 1.00 | 16.18 | A |
| ATOM | 561 | C | PRO | A | 75 | 26.810 | 20.314 | 75.412 | 1.00 | 9.11 | A |
| ATOM | 562 | O | PRO | A | 75 | 27.024 | 19.728 | 74.347 | 1.00 | 8.34 | A |
| ATOM | 563 | N | PHE | A | 76 | 27.219 | 21.561 | 75.636 | 1.00 | 5.84 | A |
| ATOM | 564 | CA | PHE | A | 76 | 27.908 | 22.301 | 74.587 | 1.00 | 7.39 | A |
| ATOM | 565 | CB | PHE | A | 76 | 27.304 | 23.701 | 74.448 | 1.00 | 7.58 | A |
| ATOM | 566 | CG | PHE | A | 76 | 25.858 | 23.679 | 74.052 | 1.00 | 8.14 | A |
| ATOM | 567 | CD1 | PHE | A | 76 | 24.857 | 23.649 | 75.014 | 1.00 | 9.90 | A |
| ATOM | 568 | CD2 | PHE | A | 76 | 25.499 | 23.595 | 72.712 | 1.00 | 8.52 | A |
| ATOM | 569 | CE1 | PHE | A | 76 | 23.516 | 23.529 | 74.647 | 1.00 | 8.95 | A |
| ATOM | 570 | CE2 | PHE | A | 76 | 24.162 | 23.472 | 72.331 | 1.00 | 7.79 | A |
| ATOM | 571 | CZ | PHE | A | 76 | 23.167 | 23.438 | 73.302 | 1.00 | 8.76 | A |
| ATOM | 572 | C | PHE | A | 76 | 29.418 | 22.376 | 74.748 | 1.00 | 7.79 | A |
| ATOM | 573 | O | PHE | A | 76 | 30.100 | 23.098 | 74.013 | 1.00 | 9.00 | A |
| ATOM | 574 | N | TYR | A | 77 | 29.945 | 21.616 | 75.700 | 1.00 | 7.33 | A |
| ATOM | 575 | CA | TYR | A | 77 | 31.389 | 21.576 | 75.913 | 1.00 | 7.72 | A |
| ATOM | 576 | CB | TYR | A | 77 | 31.724 | 20.905 | 77.249 | 1.00 | 7.26 | A |
| ATOM | 577 | CG | TYR | A | 77 | 31.455 | 21.716 | 78.499 | 1.00 | 5.81 | A |
| ATOM | 578 | CD1 | TYR | A | 77 | 30.896 | 22.992 | 78.440 | 1.00 | 5.79 | A |
| ATOM | 579 | CE1 | TYR | A | 77 | 30.650 | 23.727 | 79.614 | 1.00 | 8.02 | A |
| ATOM | 580 | CD2 | TYR | A | 77 | 31.765 | 21.190 | 79.755 | 1.00 | 5.80 | A |
| ATOM | 581 | CE2 | TYR | A | 77 | 31.525 | 21.910 | 80.926 | 1.00 | 6.27 | A |
| ATOM | 582 | CZ | TYR | A | 77 | 30.970 | 23.172 | 80.852 | 1.00 | 6.18 | A |
| ATOM | 583 | OH | TYR | A | 77 | 30.745 | 23.872 | 82.016 | 1.00 | 6.11 | A |
| ATOM | 584 | C | TYR | A | 77 | 32.025 | 20.764 | 74.783 | 1.00 | 8.63 | A |
| ATOM | 585 | O | TYR | A | 77 | 33.116 | 21.083 | 74.315 | 1.00 | 9.57 | A |
| ATOM | 586 | N | ARG | A | 78 | 31.343 | 19.710 | 74.345 | 1.00 | 9.28 | A |
| ATOM | 587 | CA | ARG | A | 78 | 31.877 | 18.867 | 73.276 | 1.00 | 12.08 | A |
| ATOM | 588 | CB | ARG | A | 78 | 30.932 | 17.695 | 72.984 | 1.00 | 15.49 | A |
| ATOM | 589 | CG | ARG | A | 78 | 31.417 | 16.799 | 71.849 | 1.00 | 22.57 | A |
| ATOM | 590 | CD | ARG | A | 78 | 30.512 | 15.600 | 71.659 | 1.00 | 27.13 | A |
| ATOM | 591 | NE | ARG | A | 78 | 30.445 | 14.792 | 72.870 | 1.00 | 31.31 | A |
| ATOM | 592 | CZ | ARG | A | 78 | 29.768 | 13.655 | 72.974 | 1.00 | 32.82 | A |
| ATOM | 593 | NH1 | ARG | A | 78 | 29.094 | 13.182 | 71.932 | 1.00 | 33.90 | A |
| ATOM | 594 | NH2 | ARG | A | 78 | 29.766 | 12.991 | 74.123 | 1.00 | 33.60 | A |
| ATOM | 595 | C | ARG | A | 78 | 32.092 | 19.674 | 72.004 | 1.00 | 10.78 | A |
| ATOM | 596 | O | ARG | A | 78 | 31.282 | 20.537 | 71.661 | 1.00 | 8.68 | A |
| ATOM | 597 | N | GLY | A | 79 | 33.197 | 19.400 | 71.317 | 1.00 | 11.29 | A |
| ATOM | 598 | CA | GLY | A | 79 | 33.499 | 20.109 | 70.086 | 1.00 | 11.30 | A |
| ATOM | 599 | C | GLY | A | 79 | 34.394 | 21.315 | 70.298 | 1.00 | 10.78 | A |
| ATOM | 600 | O | GLY | A | 79 | 34.890 | 21.905 | 69.337 | 1.00 | 11.85 | A |
| ATOM | 601 | N | SER | A | 80 | 34.591 | 21.691 | 71.559 | 1.00 | 9.57 | A |
| ATOM | 602 | CA | SER | A | 80 | 35.439 | 22.829 | 71.897 | 1.00 | 9.30 | A |
| ATOM | 603 | CB | SER | A | 80 | 35.340 | 23.154 | 73.395 | 1.00 | 7.79 | A |
| ATOM | 604 | OG | SER | A | 80 | 34.023 | 23.539 | 73.773 | 1.00 | 9.21 | A |
| ATOM | 605 | C | SER | A | 80 | 36.876 | 22.466 | 71.546 | 1.00 | 9.31 | A |
| ATOM | 606 | O | SER | A | 80 | 37.320 | 21.344 | 71.794 | 1.00 | 10.34 | A |
| ATOM | 607 | N | ASP | A | 81 | 37.601 | 23.417 | 70.966 | 1.00 | 9.29 | A |
| ATOM | 608 | CA | ASP | A | 81 | 38.984 | 23.195 | 70.575 | 1.00 | 8.50 | A |
| ATOM | 609 | CB | ASP | A | 81 | 39.251 | 23.879 | 69.233 | 1.00 | 8.43 | A |
| ATOM | 610 | CG | ASP | A | 81 | 38.395 | 23.303 | 68.121 | 1.00 | 10.96 | A |
| ATOM | 611 | OD1 | ASP | A | 81 | 38.570 | 22.107 | 67.814 | 1.00 | 11.76 | A |
| ATOM | 612 | OD2 | ASP | A | 81 | 37.542 | 24.033 | 67.568 | 1.00 | 10.77 | A |
| ATOM | 613 | C | ASP | A | 81 | 39.942 | 23.717 | 71.634 | 1.00 | 8.57 | A |
| ATOM | 614 | O | ASP | A | 81 | 41.090 | 23.271 | 71.725 | 1.00 | 9.22 | A |
| ATOM | 615 | N | CYS | A | 82 | 39.466 | 24.667 | 72.430 | 1.00 | 8.13 | A |
| ATOM | 616 | CA | CYS | A | 82 | 40.265 | 25.240 | 73.500 | 1.00 | 8.64 | A |
| ATOM | 617 | CB | CYS | A | 82 | 41.177 | 26.353 | 72.976 | 1.00 | 9.61 | A |
| ATOM | 618 | SG | CYS | A | 82 | 42.304 | 27.048 | 74.240 | 1.00 | 12.93 | A |
| ATOM | 619 | C | CYS | A | 82 | 39.329 | 25.810 | 74.546 | 1.00 | 10.29 | A |
| ATOM | 620 | O | CYS | A | 82 | 38.219 | 26.243 | 74.229 | 1.00 | 7.59 | A |
| ATOM | 621 | N | CYS | A | 83 | 39.773 | 25.789 | 75.795 | 1.00 | 10.63 | A |
| ATOM | 622 | CA | CYS | A | 83 | 38.975 | 26.324 | 76.886 | 1.00 | 11.81 | A |
| ATOM | 623 | CB | CYS | A | 83 | 38.623 | 25.219 | 77.887 | 1.00 | 13.61 | A |
| ATOM | 624 | SG | CYS | A | 83 | 37.813 | 25.817 | 79.404 | 1.00 | 20.11 | A |
| ATOM | 625 | C | CYS | A | 83 | 39.751 | 27.422 | 77.594 | 1.00 | 12.97 | A |
| ATOM | 626 | O | CYS | A | 83 | 40.905 | 27.232 | 77.981 | 1.00 | 9.87 | A |
| ATOM | 627 | N | LEU | A | 84 | 39.118 | 28.581 | 77.735 | 1.00 | 11.80 | A |
| ATOM | 628 | CA | LEU | A | 84 | 39.728 | 29.704 | 78.427 | 1.00 | 14.00 | A |
| ATOM | 629 | CB | LEU | A | 84 | 39.404 | 31.028 | 77.731 | 1.00 | 15.59 | A |
| ATOM | 630 | CG | LEU | A | 84 | 39.956 | 31.294 | 76.330 | 1.00 | 20.45 | A |
| ATOM | 631 | CD1 | LEU | A | 84 | 39.520 | 32.679 | 75.880 | 1.00 | 20.50 | A |
| ATOM | 632 | CD2 | LEU | A | 84 | 41.468 | 31.199 | 76.327 | 1.00 | 19.66 | A |
| ATOM | 633 | C | LEU | A | 84 | 39.113 | 29.701 | 79.815 | 1.00 | 13.67 | A |
| ATOM | 634 | O | LEU | A | 84 | 37.980 | 30.142 | 80.000 | 1.00 | 14.19 | A |
| ATOM | 635 | N | LEU | A | 85 | 39.855 | 29.176 | 80.781 | 1.00 | 11.60 | A |
| ATOM | 636 | CA | LEU | A | 85 | 39.383 | 29.106 | 82.151 | 1.00 | 10.31 | A |
| ATOM | 637 | CB | LEU | A | 85 | 40.010 | 27.899 | 82.850 | 1.00 | 9.76 | A |
| ATOM | 638 | CG | LEU | A | 85 | 39.513 | 27.586 | 84.256 | 1.00 | 13.44 | A |
| ATOM | 639 | CD1 | LEU | A | 85 | 37.993 | 27.648 | 84.303 | 1.00 | 14.52 | A |
| ATOM | 640 | CD2 | LEU | A | 85 | 40.020 | 26.212 | 84.664 | 1.00 | 14.57 | A |
| ATOM | 641 | C | LEU | A | 85 | 39.774 | 30.406 | 82.840 | 1.00 | 9.29 | A |
| ATOM | 642 | O | LEU | A | 85 | 40.955 | 30.744 | 82.921 | 1.00 | 6.24 | A |
| ATOM | 643 | N | THR | A | 86 | 38.776 | 31.133 | 83.332 | 1.00 | 8.36 | A |
| ATOM | 644 | CA | THR | A | 86 | 39.013 | 32.420 | 83.967 | 1.00 | 8.75 | A |
| ATOM | 645 | CB | THR | A | 86 | 38.229 | 33.538 | 83.228 | 1.00 | 10.54 | A |
| ATOM | 646 | OG1 | THR | A | 86 | 38.608 | 33.563 | 81.848 | 1.00 | 12.78 | A |
| ATOM | 647 | CG2 | THR | A | 86 | 38.517 | 34.893 | 83.843 | 1.00 | 10.99 | A |
| ATOM | 648 | C | THR | A | 86 | 38.640 | 32.516 | 85.443 | 1.00 | 8.35 | A |
| ATOM | 649 | O | THR | A | 86 | 37.653 | 31.926 | 85.895 | 1.00 | 5.92 | A |
| ATOM | 650 | N | PHE | A | 87 | 39.454 | 33.257 | 86.188 | 1.00 | 8.15 | A |
| ATOM | 651 | CA | PHE | A | 87 | 39.185 | 33.525 | 87.595 | 1.00 | 7.66 | A |
| ATOM | 652 | CB | PHE | A | 87 | 40.053 | 32.669 | 88.534 | 1.00 | 7.35 | A |
| ATOM | 653 | CG | PHE | A | 87 | 41.514 | 33.029 | 88.533 | 1.00 | 6.28 | A |
| ATOM | 654 | CD1 | PHE | A | 87 | 42.375 | 32.500 | 87.578 | 1.00 | 6.23 | A |
| ATOM | 655 | CD2 | PHE | A | 87 | 42.034 | 33.890 | 89.504 | 1.00 | 5.83 | A |
| ATOM | 656 | CE1 | PHE | A | 87 | 43.738 | 32.818 | 87.582 | 1.00 | 5.13 | A |
| ATOM | 657 | CE2 | PHE | A | 87 | 43.392 | 34.215 | 89.517 | 1.00 | 4.88 | A |
| ATOM | 658 | CZ | PHE | A | 87 | 44.248 | 33.676 | 88.551 | 1.00 | 4.44 | A |
| ATOM | 659 | C | PHE | A | 87 | 39.496 | 35.002 | 87.778 | 1.00 | 8.21 | A |
| ATOM | 660 | O | PHE | A | 87 | 40.091 | 35.629 | 86.893 | 1.00 | 8.99 | A |
| ATOM | 661 | N | SER | A | 88 | 39.070 | 35.567 | 88.900 | 1.00 | 6.56 | A |
| ATOM | 662 | CA | SER | A | 88 | 39.319 | 36.972 | 89.191 | 1.00 | 8.91 | A |
| ATOM | 663 | CB | SER | A | 88 | 38.029 | 37.644 | 89.673 | 1.00 | 9.54 | A |
| ATOM | 664 | OG | SER | A | 88 | 38.305 | 38.939 | 90.176 | 1.00 | 12.32 | A |
| ATOM | 665 | C | SER | A | 88 | 40.387 | 37.055 | 90.276 | 1.00 | 7.36 | A |
| ATOM | 666 | O | SER | A | 88 | 40.280 | 36.393 | 91.302 | 1.00 | 7.03 | A |
| ATOM | 667 | N | VAL | A | 89 | 41.415 | 37.868 | 90.052 | 1.00 | 9.18 | A |
| ATOM | 668 | CA | VAL | A | 89 | 42.492 | 37.992 | 91.030 | 1.00 | 10.18 | A |
| ATOM | 669 | CB | VAL | A | 89 | 43.679 | 38.803 | 90.464 | 1.00 | 9.74 | A |
| ATOM | 670 | CG1 | VAL | A | 89 | 44.224 | 38.107 | 89.218 | 1.00 | 9.11 | A |
| ATOM | 671 | CG2 | VAL | A | 89 | 43.250 | 40.220 | 90.159 | 1.00 | 8.85 | A |
| ATOM | 672 | C | VAL | A | 89 | 42.062 | 38.597 | 92.365 | 1.00 | 11.18 | A |
| ATOM | 673 | O | VAL | A | 89 | 42.821 | 38.566 | 93.334 | 1.00 | 11.04 | A |
| ATOM | 674 | N | ASP | A | 90 | 40.855 | 39.149 | 92.427 | 1.00 | 11.47 | A |
| ATOM | 675 | CA | ASP | A | 90 | 40.381 | 39.707 | 93.687 | 1.00 | 13.90 | A |
| ATOM | 676 | CB | ASP | A | 90 | 39.800 | 41.117 | 93.480 | 1.00 | 16.59 | A |
| ATOM | 677 | CG | ASP | A | 90 | 38.354 | 41.104 | 93.035 | 1.00 | 21.54 | A |
| ATOM | 678 | OD1 | ASP | A | 90 | 37.988 | 40.248 | 92.207 | 1.00 | 23.51 | A |
| ATOM | 679 | OD2 | ASP | A | 90 | 37.579 | 41.967 | 93.509 | 1.00 | 24.35 | A |
| ATOM | 680 | C | ASP | A | 90 | 39.342 | 38.762 | 94.299 | 1.00 | 13.44 | A |
| ATOM | 681 | O | ASP | A | 90 | 38.606 | 39.135 | 95.214 | 1.00 | 12.66 | A |
| ATOM | 682 | N | ASP | A | 91 | 39.300 | 37.528 | 93.796 | 1.00 | 12.27 | A |
| ATOM | 683 | CA | ASP | A | 91 | 38.359 | 36.529 | 94.299 | 1.00 | 12.01 | A |
| ATOM | 684 | CB | ASP | A | 91 | 37.127 | 36.463 | 93.389 | 1.00 | 14.88 | A |
| ATOM | 685 | CG | ASP | A | 91 | 35.999 | 35.652 | 93.998 | 1.00 | 18.25 | A |
| ATOM | 686 | OD1 | ASP | A | 91 | 36.177 | 34.433 | 94.201 | 1.00 | 18.76 | A |
| ATOM | 687 | OD2 | ASP | A | 91 | 34.935 | 36.240 | 94.284 | 1.00 | 23.64 | A |
| ATOM | 688 | C | ASP | A | 91 | 39.017 | 35.152 | 94.386 | 1.00 | 10.93 | A |
| ATOM | 689 | O | ASP | A | 91 | 38.997 | 34.379 | 93.426 | 1.00 | 8.79 | A |
| ATOM | 690 | N | SER | A | 92 | 39.598 | 34.846 | 95.544 | 1.00 | 10.34 | A |
| ATOM | 691 | CA | SER | A | 92 | 40.275 | 33.569 | 95.739 | 1.00 | 10.40 | A |
| ATOM | 692 | CB | SER | A | 92 | 40.926 | 33.516 | 97.129 | 1.00 | 11.90 | A |
| ATOM | 693 | OG | SER | A | 92 | 39.970 | 33.673 | 98.156 | 1.00 | 16.18 | A |
| ATOM | 694 | C | SER | A | 92 | 39.374 | 32.355 | 95.535 | 1.00 | 9.68 | A |
| ATOM | 695 | O | SER | A | 92 | 39.851 | 31.285 | 95.158 | 1.00 | 9.35 | A |
| ATOM | 696 | N | GLN | A | 93 | 38.074 | 32.512 | 95.767 | 1.00 | 9.53 | A |
| ATOM | 697 | CA | GLN | A | 93 | 37.153 | 31.398 | 95.577 | 1.00 | 9.69 | A |
| ATOM | 698 | CB | GLN | A | 93 | 35.740 | 31.787 | 96.011 | 1.00 | 13.40 | A |
| ATOM | 699 | CG | GLN | A | 93 | 34.731 | 30.662 | 95.858 | 1.00 | 18.35 | A |
| ATOM | 700 | CD | GLN | A | 93 | 33.323 | 31.093 | 96.217 | 1.00 | 20.38 | A |
| ATOM | 701 | OE1 | GLN | A | 93 | 32.780 | 32.030 | 95.630 | 1.00 | 25.11 | A |
| ATOM | 702 | NE2 | GLN | A | 93 | 32.724 | 30.411 | 97.186 | 1.00 | 24.05 | A |
| ATOM | 703 | C | GLN | A | 93 | 37.138 | 30.988 | 94.108 | 1.00 | 8.52 | A |
| ATOM | 704 | O | GLN | A | 93 | 37.180 | 29.802 | 93.786 | 1.00 | 6.46 | A |
| ATOM | 705 | N | SER | A | 94 | 37.086 | 31.974 | 93.217 | 1.00 | 8.02 | A |
| ATOM | 706 | CA | SER | A | 94 | 37.068 | 31.692 | 91.784 | 1.00 | 5.55 | A |
| ATOM | 707 | CB | SER | A | 94 | 36.860 | 32.989 | 90.985 | 1.00 | 4.92 | A |
| ATOM | 708 | OG | SER | A | 94 | 38.006 | 33.824 | 91.017 | 1.00 | 5.72 | A |
| ATOM | 709 | C | SER | A | 94 | 38.367 | 31.007 | 91.362 | 1.00 | 5.38 | A |
| ATOM | 710 | O | SER | A | 94 | 38.380 | 30.218 | 90.418 | 1.00 | 6.20 | A |
| ATOM | 711 | N | PHE | A | 95 | 39.456 | 31.311 | 92.064 | 1.00 | 4.64 | A |
| ATOM | 712 | CA | PHE | A | 95 | 40.754 | 30.699 | 91.778 | 1.00 | 6.60 | A |
| ATOM | 713 | CB | PHE | A | 95 | 41.875 | 31.515 | 92.438 | 1.00 | 6.85 | A |
| ATOM | 714 | CG | PHE | A | 95 | 43.232 | 30.885 | 92.331 | 1.00 | 7.79 | A |
| ATOM | 715 | CD1 | PHE | A | 95 | 43.855 | 30.740 | 91.093 | 1.00 | 9.08 | A |
| ATOM | 716 | CD2 | PHE | A | 95 | 43.888 | 30.424 | 93.468 | 1.00 | 9.93 | A |
| ATOM | 717 | CE1 | PHE | A | 95 | 45.117 | 30.143 | 90.991 | 1.00 | 10.78 | A |
| ATOM | 718 | CE2 | PHE | A | 95 | 45.153 | 29.825 | 93.375 | 1.00 | 11.05 | A |
| ATOM | 719 | CZ | PHE | A | 95 | 45.764 | 29.686 | 92.134 | 1.00 | 10.14 | A |
| ATOM | 720 | C | PHE | A | 95 | 40.754 | 29.263 | 92.320 | 1.00 | 6.44 | A |
| ATOM | 721 | O | PHE | A | 95 | 41.220 | 28.337 | 91.655 | 1.00 | 4.89 | A |
| ATOM | 722 | N | GLN | A | 96 | 40.228 | 29.085 | 93.528 | 1.00 | 5.38 | A |
| ATOM | 723 | CA | GLN | A | 96 | 40.165 | 27.760 | 94.142 | 1.00 | 8.24 | A |
| ATOM | 724 | CB | GLN | A | 96 | 39.580 | 27.856 | 95.556 | 1.00 | 9.01 | A |
| ATOM | 725 | CG | GLN | A | 96 | 40.522 | 28.473 | 96.581 | 1.00 | 13.20 | A |
| ATOM | 726 | CD | GLN | A | 96 | 39.783 | 29.090 | 97.753 | 1.00 | 16.63 | A |
| ATOM | 727 | OE1 | GLN | A | 96 | 38.711 | 28.621 | 98.148 | 1.00 | 17.27 | A |
| ATOM | 728 | NE2 | GLN | A | 96 | 40.360 | 30.141 | 98.327 | 1.00 | 18.42 | A |
| ATOM | 729 | C | GLN | A | 96 | 39.312 | 26.812 | 93.296 | 1.00 | 7.54 | A |
| ATOM | 730 | O | GLN | A | 96 | 39.538 | 25.604 | 93.288 | 1.00 | 8.95 | A |
| ATOM | 731 | N | ASN | A | 97 | 38.342 | 27.370 | 92.574 | 1.00 | 5.72 | A |
| ATOM | 732 | CA | ASN | A | 97 | 37.453 | 26.573 | 91.737 | 1.00 | 5.45 | A |
| ATOM | 733 | CB | ASN | A | 97 | 36.127 | 27.317 | 91.531 | 1.00 | 6.64 | A |
| ATOM | 734 | CG | ASN | A | 97 | 35.201 | 27.203 | 92.730 | 1.00 | 8.78 | A |
| ATOM | 735 | OD1 | ASN | A | 97 | 34.287 | 28.010 | 92.905 | 1.00 | 11.45 | A |
| ATOM | 736 | ND2 | ASN | A | 97 | 35.429 | 26.190 | 93.561 | 1.00 | 9.35 | A |
| ATOM | 737 | C | ASN | A | 97 | 38.043 | 26.182 | 90.384 | 1.00 | 5.94 | A |
| ATOM | 738 | O | ASN | A | 97 | 37.393 | 25.491 | 89.599 | 1.00 | 5.21 | A |
| ATOM | 739 | N | LEU | A | 98 | 39.269 | 26.608 | 90.101 | 1.00 | 5.46 | A |
| ATOM | 740 | CA | LEU | A | 98 | 39.878 | 26.257 | 88.826 | 1.00 | 5.83 | A |
| ATOM | 741 | CB | LEU | A | 98 | 41.290 | 26.838 | 88.716 | 1.00 | 6.89 | A |
| ATOM | 742 | CG | LEU | A | 98 | 41.392 | 28.339 | 88.459 | 1.00 | 6.41 | A |
| ATOM | 743 | CD1 | LEU | A | 98 | 42.860 | 28.731 | 88.391 | 1.00 | 8.56 | A |
| ATOM | 744 | CD2 | LEU | A | 98 | 40.689 | 28.699 | 87.149 | 1.00 | 9.19 | A |
| ATOM | 745 | C | LEU | A | 98 | 39.937 | 24.747 | 88.640 | 1.00 | 5.95 | A |
| ATOM | 746 | O | LEU | A | 98 | 39.640 | 24.241 | 87.560 | 1.00 | 6.55 | A |
| ATOM | 747 | N | SER | A | 99 | 40.322 | 24.026 | 89.690 | 1.00 | 6.66 | A |
| ATOM | 748 | CA | SER | A | 99 | 40.411 | 22.574 | 89.598 | 1.00 | 8.64 | A |
| ATOM | 749 | CB | SER | A | 99 | 40.948 | 21.980 | 90.902 | 1.00 | 11.10 | A |
| ATOM | 750 | OG | SER | A | 99 | 40.030 | 22.159 | 91.964 | 1.00 | 13.75 | A |
| ATOM | 751 | C | SER | A | 99 | 39.037 | 21.989 | 89.292 | 1.00 | 8.06 | A |
| ATOM | 752 | O | SER | A | 99 | 38.916 | 21.066 | 88.484 | 1.00 | 7.52 | A |
| ATOM | 753 | N | ASN | A | 100 | 38.009 | 22.533 | 89.942 | 1.00 | 8.17 | A |
| ATOM | 754 | CA | ASN | A | 100 | 36.639 | 22.072 | 89.739 | 1.00 | 8.84 | A |
| ATOM | 755 | CB | ASN | A | 100 | 35.657 | 22.845 | 90.631 | 1.00 | 9.97 | A |
| ATOM | 756 | CG | ASN | A | 100 | 35.919 | 22.645 | 92.117 | 1.00 | 13.07 | A |
| ATOM | 757 | OD1 | ASN | A | 100 | 35.022 | 22.251 | 92.872 | 1.00 | 15.25 | A |
| ATOM | 758 | ND2 | ASN | A | 100 | 37.141 | 22.927 | 92.546 | 1.00 | 13.40 | A |
| ATOM | 759 | C | ASN | A | 100 | 36.234 | 22.270 | 88.282 | 1.00 | 7.78 | A |
| ATOM | 760 | O | ASN | A | 100 | 35.655 | 21.376 | 87.663 | 1.00 | 6.56 | A |
| ATOM | 761 | N | TRP | A | 101 | 36.534 | 23.448 | 87.739 | 1.00 | 6.12 | A |
| ATOM | 762 | CA | TRP | A | 101 | 36.186 | 23.744 | 86.353 | 1.00 | 5.29 | A |
| ATOM | 763 | CB | TRP | A | 101 | 36.522 | 25.196 | 86.004 | 1.00 | 5.82 | A |
| ATOM | 764 | CG | TRP | A | 101 | 35.499 | 26.178 | 86.485 | 1.00 | 4.69 | A |
| ATOM | 765 | CD2 | TRP | A | 101 | 34.165 | 26.348 | 85.981 | 1.00 | 7.09 | A |
| ATOM | 766 | CE2 | TRP | A | 101 | 33.562 | 27.379 | 86.736 | 1.00 | 5.99 | A |
| ATOM | 767 | CE3 | TRP | A | 101 | 33.423 | 25.730 | 84.967 | 1.00 | 7.68 | A |
| ATOM | 768 | CD1 | TRP | A | 101 | 35.644 | 27.085 | 87.492 | 1.00 | 6.25 | A |
| ATOM | 769 | NE1 | TRP | A | 101 | 34.488 | 27.810 | 87.649 | 1.00 | 6.24 | A |
| ATOM | 770 | CZ2 | TRP | A | 101 | 32.250 | 27.808 | 86.509 | 1.00 | 6.85 | A |
| ATOM | 771 | CZ3 | TRP | A | 101 | 32.114 | 26.159 | 84.740 | 1.00 | 8.00 | A |
| ATOM | 772 | CH2 | TRP | A | 101 | 31.545 | 27.187 | 85.508 | 1.00 | 6.74 | A |
| ATOM | 773 | C | TRP | A | 101 | 36.878 | 22.817 | 85.367 | 1.00 | 5.85 | A |
| ATOM | 774 | O | TRP | A | 101 | 36.266 | 22.368 | 84.399 | 1.00 | 4.23 | A |
| ATOM | 775 | N | LYS | A | 102 | 38.158 | 22.541 | 85.597 | 1.00 | 5.20 | A |
| ATOM | 776 | CA | LYS | A | 102 | 38.887 | 21.657 | 84.699 | 1.00 | 6.11 | A |
| ATOM | 777 | CB | LYS | A | 102 | 40.348 | 21.535 | 85.133 | 1.00 | 6.32 | A |
| ATOM | 778 | CG | LYS | A | 102 | 41.125 | 20.478 | 84.350 | 1.00 | 6.75 | A |
| ATOM | 779 | CD | LYS | A | 102 | 42.613 | 20.532 | 84.645 | 1.00 | 9.97 | A |
| ATOM | 780 | CE | LYS | A | 102 | 43.335 | 19.369 | 83.981 | 1.00 | 9.75 | A |
| ATOM | 781 | NZ | LYS | A | 102 | 44.794 | 19.402 | 84.253 | 1.00 | 11.24 | A |
| ATOM | 782 | C | LYS | A | 102 | 38.237 | 20.276 | 84.667 | 1.00 | 6.21 | A |
| ATOM | 783 | O | LYS | A | 102 | 37.995 | 19.721 | 83.592 | 1.00 | 5.97 | A |
| ATOM | 784 | N | LYS | A | 103 | 37.945 | 19.729 | 85.843 | 1.00 | 7.57 | A |
| ATOM | 785 | CA | LYS | A | 103 | 37.325 | 18.412 | 85.935 | 1.00 | 7.56 | A |
| ATOM | 786 | CB | LYS | A | 103 | 37.235 | 17.964 | 87.398 | 1.00 | 8.92 | A |
| ATOM | 787 | CG | LYS | A | 103 | 38.584 | 17.521 | 87.967 | 1.00 | 12.81 | A |
| ATOM | 788 | CD | LYS | A | 103 | 38.431 | 16.758 | 89.277 | 1.00 | 19.28 | A |
| ATOM | 789 | CE | LYS | A | 103 | 38.600 | 17.659 | 90.487 | 1.00 | 21.76 | A |
| ATOM | 790 | NZ | LYS | A | 103 | 40.010 | 18.124 | 90.637 | 1.00 | 20.12 | A |
| ATOM | 791 | C | LYS | A | 103 | 35.948 | 18.375 | 85.280 | 1.00 | 6.98 | A |
| ATOM | 792 | O | LYS | A | 103 | 35.617 | 17.415 | 84.580 | 1.00 | 6.82 | A |
| ATOM | 793 | N | GLU | A | 104 | 35.157 | 19.422 | 85.496 | 1.00 | 5.59 | A |
| ATOM | 794 | CA | GLU | A | 104 | 33.824 | 19.509 | 84.909 | 1.00 | 6.06 | A |
| ATOM | 795 | CB | GLU | A | 104 | 33.105 | 20.786 | 85.375 | 1.00 | 7.01 | A |
| ATOM | 796 | CG | GLU | A | 104 | 31.695 | 20.933 | 84.797 | 1.00 | 7.92 | A |
| ATOM | 797 | CD | GLU | A | 104 | 31.004 | 22.224 | 85.206 | 1.00 | 10.80 | A |
| ATOM | 798 | OE1 | GLU | A | 104 | 30.954 | 22.526 | 86.420 | 1.00 | 11.56 | A |
| ATOM | 799 | OE2 | GLU | A | 104 | 30.497 | 22.936 | 84.313 | 1.00 | 9.80 | A |
| ATOM | 800 | C | GLU | A | 104 | 33.920 | 19.513 | 83.386 | 1.00 | 6.43 | A |
| ATOM | 801 | O | GLU | A | 104 | 33.193 | 18.783 | 82.706 | 1.00 | 6.35 | A |
| ATOM | 802 | N | PHE | A | 105 | 34.816 | 20.334 | 82.845 | 1.00 | 5.49 | A |
| ATOM | 803 | CA | PHE | A | 105 | 34.970 | 20.401 | 81.395 | 1.00 | 4.74 | A |
| ATOM | 804 | CB | PHE | A | 105 | 36.016 | 21.446 | 80.981 | 1.00 | 3.79 | A |
| ATOM | 805 | CG | PHE | A | 105 | 36.206 | 21.533 | 79.491 | 1.00 | 4.14 | A |
| ATOM | 806 | CD1 | PHE | A | 105 | 35.272 | 22.196 | 78.696 | 1.00 | 5.78 | A |
| ATOM | 807 | CD2 | PHE | A | 105 | 37.278 | 20.897 | 78.872 | 1.00 | 7.46 | A |
| ATOM | 808 | CE1 | PHE | A | 105 | 35.401 | 22.224 | 77.310 | 1.00 | 5.94 | A |
| ATOM | 809 | CE2 | PHE | A | 105 | 37.415 | 20.918 | 77.485 | 1.00 | 7.97 | A |
| ATOM | 810 | CZ | PHE | A | 105 | 36.470 | 21.584 | 76.703 | 1.00 | 6.65 | A |
| ATOM | 811 | C | PHE | A | 105 | 35.392 | 19.059 | 80.816 | 1.00 | 6.18 | A |
| ATOM | 812 | O | PHE | A | 105 | 34.786 | 18.566 | 79.863 | 1.00 | 5.11 | A |
| ATOM | 813 | N | ILE | A | 106 | 36.440 | 18.474 | 81.391 | 1.00 | 6.29 | A |
| ATOM | 814 | CA | ILE | A | 106 | 36.943 | 17.193 | 80.915 | 1.00 | 7.42 | A |
| ATOM | 815 | CB | ILE | A | 106 | 38.193 | 16.760 | 81.727 | 1.00 | 8.46 | A |
| ATOM | 816 | CG2 | ILE | A | 106 | 38.539 | 15.297 | 81.441 | 1.00 | 8.88 | A |
| ATOM | 817 | CG1 | ILE | A | 106 | 39.369 | 17.676 | 81.372 | 1.00 | 10.62 | A |
| ATOM | 818 | CD1 | ILE | A | 106 | 40.575 | 17.508 | 82.268 | 1.00 | 13.35 | A |
| ATOM | 819 | C | ILE | A | 106 | 35.880 | 16.103 | 80.986 | 1.00 | 8.19 | A |
| ATOM | 820 | O | ILE | A | 106 | 35.729 | 15.303 | 80.056 | 1.00 | 7.67 | A |
| ATOM | 821 | N | TYR | A | 107 | 35.131 | 16.077 | 82.079 | 1.00 | 7.20 | A |
| ATOM | 822 | CA | TYR | A | 107 | 34.098 | 15.065 | 82.247 | 1.00 | 7.51 | A |
| ATOM | 823 | CB | TYR | A | 107 | 33.445 | 15.196 | 83.624 | 1.00 | 9.59 | A |
| ATOM | 824 | CG | TYR | A | 107 | 32.515 | 14.047 | 83.913 | 1.00 | 10.97 | A |
| ATOM | 825 | CD1 | TYR | A | 107 | 33.022 | 12.771 | 84.146 | 1.00 | 15.01 | A |
| ATOM | 826 | CE1 | TYR | A | 107 | 32.176 | 11.685 | 84.346 | 1.00 | 15.15 | A |
| ATOM | 827 | CD2 | TYR | A | 107 | 31.132 | 14.216 | 83.892 | 1.00 | 12.44 | A |
| ATOM | 828 | CE2 | TYR | A | 107 | 30.274 | 13.132 | 84.091 | 1.00 | 14.95 | A |
| ATOM | 829 | CZ | TYR | A | 107 | 30.809 | 11.872 | 84.315 | 1.00 | 15.17 | A |
| ATOM | 830 | OH | TYR | A | 107 | 29.980 | 10.789 | 84.503 | 1.00 | 16.94 | A |
| ATOM | 831 | C | TYR | A | 107 | 33.005 | 15.127 | 81.180 | 1.00 | 8.28 | A |
| ATOM | 832 | O | TYR | A | 107 | 32.591 | 14.101 | 80.633 | 1.00 | 7.93 | A |
| ATOM | 833 | N | TYR | A | 108 | 32.539 | 16.332 | 80.883 | 1.00 | 8.03 | A |
| ATOM | 834 | CA | TYR | A | 108 | 31.465 | 16.516 | 79.915 | 1.00 | 8.20 | A |
| ATOM | 835 | CB | TYR | A | 108 | 30.574 | 17.677 | 80.368 | 1.00 | 8.76 | A |
| ATOM | 836 | CG | TYR | A | 108 | 29.730 | 17.358 | 81.587 | 1.00 | 6.76 | A |
| ATOM | 837 | CD1 | TYR | A | 108 | 28.640 | 16.494 | 81.495 | 1.00 | 10.71 | A |
| ATOM | 838 | CE1 | TYR | A | 108 | 27.849 | 16.205 | 82.610 | 1.00 | 10.86 | A |
| ATOM | 839 | CD2 | TYR | A | 108 | 30.016 | 17.926 | 82.828 | 1.00 | 8.16 | A |
| ATOM | 840 | CE2 | TYR | A | 108 | 29.236 | 17.644 | 83.948 | 1.00 | 8.62 | A |
| ATOM | 841 | CZ | TYR | A | 108 | 28.152 | 16.785 | 83.831 | 1.00 | 10.99 | A |
| ATOM | 842 | OH | TYR | A | 108 | 27.358 | 16.521 | 84.926 | 1.00 | 12.71 | A |
| ATOM | 843 | C | TYR | A | 108 | 31.874 | 16.734 | 78.460 | 1.00 | 9.49 | A |
| ATOM | 844 | O | TYR | A | 108 | 31.067 | 16.518 | 77.556 | 1.00 | 8.85 | A |
| ATOM | 845 | N | ALA | A | 109 | 33.111 | 17.156 | 78.230 | 1.00 | 10.91 | A |
| ATOM | 846 | CA | ALA | A | 109 | 33.578 | 17.426 | 76.872 | 1.00 | 15.15 | A |
| ATOM | 847 | CB | ALA | A | 109 | 34.807 | 18.331 | 76.913 | 1.00 | 15.49 | A |
| ATOM | 848 | C | ALA | A | 109 | 33.881 | 16.181 | 76.047 | 1.00 | 18.54 | A |
| ATOM | 849 | O | ALA | A | 109 | 33.718 | 16.191 | 74.829 | 1.00 | 18.46 | A |
| ATOM | 850 | N | ASP | A | 110 | 34.329 | 15.121 | 76.708 | 1.00 | 22.15 | A |
| ATOM | 851 | CA | ASP | A | 110 | 34.662 | 13.877 | 76.023 | 1.00 | 27.39 | A |
| ATOM | 852 | CB | ASP | A | 110 | 33.386 | 13.200 | 75.512 | 1.00 | 29.86 | A |
| ATOM | 853 | CG | ASP | A | 110 | 33.642 | 11.804 | 74.974 | 1.00 | 33.17 | A |
| ATOM | 854 | OD1 | ASP | A | 110 | 34.260 | 11.678 | 73.894 | 1.00 | 34.48 | A |
| ATOM | 855 | OD2 | ASP | A | 110 | 33.231 | 10.827 | 75.639 | 1.00 | 35.28 | A |
| ATOM | 856 | C | ASP | A | 110 | 35.622 | 14.139 | 74.861 | 1.00 | 29.44 | A |
| ATOM | 857 | O | ASP | A | 110 | 35.284 | 13.938 | 73.693 | 1.00 | 30.05 | A |
| ATOM | 858 | N | VAL | A | 111 | 36.823 | 14.598 | 75.194 | 1.00 | 31.11 | A |
| ATOM | 859 | CA | VAL | A | 111 | 37.840 | 14.882 | 74.191 | 1.00 | 33.32 | A |
| ATOM | 860 | CB | VAL | A | 111 | 38.744 | 16.052 | 74.635 | 1.00 | 33.82 | A |
| ATOM | 861 | CG1 | VAL | A | 111 | 39.856 | 16.273 | 73.622 | 1.00 | 35.71 | A |
| ATOM | 862 | CG2 | VAL | A | 111 | 37.910 | 17.315 | 74.790 | 1.00 | 33.26 | A |
| ATOM | 863 | C | VAL | A | 111 | 38.696 | 13.637 | 73.976 | 1.00 | 34.48 | A |
| ATOM | 864 | O | VAL | A | 111 | 38.878 | 12.836 | 74.894 | 1.00 | 33.92 | A |
| ATOM | 865 | N | LYS | A | 112 | 39.214 | 13.480 | 72.760 | 1.00 | 36.84 | A |
| ATOM | 866 | CA | LYS | A | 112 | 40.048 | 12.332 | 72.418 | 1.00 | 38.77 | A |
| ATOM | 867 | CB | LYS | A | 112 | 40.542 | 12.446 | 70.971 | 1.00 | 39.52 | A |
| ATOM | 868 | CG | LYS | A | 112 | 39.473 | 12.160 | 69.926 | 1.00 | 40.28 | A |
| ATOM | 869 | CD | LYS | A | 112 | 38.981 | 10.721 | 70.029 | 1.00 | 40.27 | A |
| ATOM | 870 | CE | LYS | A | 112 | 37.922 | 10.416 | 68.982 | 1.00 | 39.96 | A |
| ATOM | 871 | NZ | LYS | A | 112 | 37.446 | 9.007 | 69.072 | 1.00 | 40.41 | A |
| ATOM | 872 | C | LYS | A | 112 | 41.239 | 12.168 | 73.355 | 1.00 | 39.37 | A |
| ATOM | 873 | O | LYS | A | 112 | 41.574 | 11.050 | 73.749 | 1.00 | 40.01 | A |
| ATOM | 874 | N | GLU | A | 113 | 41.878 | 13.278 | 73.710 | 1.00 | 39.64 | A |
| ATOM | 875 | CA | GLU | A | 113 | 43.025 | 13.233 | 74.609 | 1.00 | 40.51 | A |
| ATOM | 876 | CB | GLU | A | 113 | 44.326 | 13.376 | 73.817 | 1.00 | 41.23 | A |
| ATOM | 877 | CG | GLU | A | 113 | 45.582 | 13.244 | 74.665 | 1.00 | 43.89 | A |
| ATOM | 878 | CD | GLU | A | 113 | 45.608 | 11.960 | 75.476 | 1.00 | 45.56 | A |
| ATOM | 879 | OE1 | GLU | A | 113 | 44.792 | 11.831 | 76.414 | 1.00 | 46.10 | A |
| ATOM | 880 | OE2 | GLU | A | 113 | 46.442 | 11.078 | 75.172 | 1.00 | 46.43 | A |
| ATOM | 881 | C | GLU | A | 113 | 42.941 | 14.329 | 75.667 | 1.00 | 40.06 | A |
| ATOM | 882 | O | GLU | A | 113 | 43.484 | 15.421 | 75.491 | 1.00 | 40.84 | A |
| ATOM | 883 | N | PRO | A | 114 | 42.263 | 14.041 | 76.791 | 1.00 | 39.18 | A |
| ATOM | 884 | CD | PRO | A | 114 | 41.657 | 12.739 | 77.123 | 1.00 | 38.11 | A |
| ATOM | 885 | CA | PRO | A | 114 | 42.091 | 14.989 | 77.897 | 1.00 | 38.19 | A |
| ATOM | 886 | CB | PRO | A | 114 | 41.183 | 14.229 | 78.861 | 1.00 | 38.04 | A |
| ATOM | 887 | CG | PRO | A | 114 | 41.572 | 12.804 | 78.627 | 1.00 | 38.50 | A |
| ATOM | 888 | C | PRO | A | 114 | 43.393 | 15.434 | 78.551 | 1.00 | 37.31 | A |
| ATOM | 889 | O | PRO | A | 114 | 43.510 | 16.569 | 79.014 | 1.00 | 36.85 | A |
| ATOM | 890 | N | GLU | A | 115 | 44.372 | 14.538 | 78.587 | 1.00 | 37.36 | A |
| ATOM | 891 | CA | GLU | A | 115 | 45.662 | 14.849 | 79.189 | 1.00 | 36.30 | A |
| ATOM | 892 | CB | GLU | A | 115 | 46.561 | 13.608 | 79.185 | 1.00 | 39.14 | A |
| ATOM | 893 | CG | GLU | A | 115 | 46.182 | 12.546 | 80.209 | 1.00 | 42.09 | A |
| ATOM | 894 | CD | GLU | A | 115 | 44.742 | 12.088 | 80.078 | 1.00 | 44.46 | A |
| ATOM | 895 | OE1 | GLU | A | 115 | 44.353 | 11.653 | 78.972 | 1.00 | 46.53 | A |
| ATOM | 896 | OE2 | GLU | A | 115 | 44.000 | 12.160 | 81.082 | 1.00 | 45.36 | A |
| ATOM | 897 | C | GLU | A | 115 | 46.378 | 15.994 | 78.478 | 1.00 | 34.13 | A |
| ATOM | 898 | O | GLU | A | 115 | 47.114 | 16.755 | 79.107 | 1.00 | 35.12 | A |
| ATOM | 899 | N | SER | A | 116 | 46.158 | 16.123 | 77.173 | 1.00 | 31.01 | A |
| ATOM | 900 | CA | SER | A | 116 | 46.818 | 17.172 | 76.403 | 1.00 | 27.41 | A |
| ATOM | 901 | CB | SER | A | 116 | 47.627 | 16.548 | 75.264 | 1.00 | 27.14 | A |
| ATOM | 902 | OG | SER | A | 116 | 46.778 | 15.885 | 74.348 | 1.00 | 29.53 | A |
| ATOM | 903 | C | SER | A | 116 | 45.890 | 18.239 | 75.826 | 1.00 | 24.04 | A |
| ATOM | 904 | O | SER | A | 116 | 46.306 | 19.021 | 74.972 | 1.00 | 23.61 | A |
| ATOM | 905 | N | PHE | A | 117 | 44.642 | 18.278 | 76.280 | 1.00 | 20.03 | A |
| ATOM | 906 | CA | PHE | A | 117 | 43.704 | 19.277 | 75.775 | 1.00 | 17.14 | A |
| ATOM | 907 | CB | PHE | A | 117 | 42.293 | 19.007 | 76.304 | 1.00 | 16.11 | A |
| ATOM | 908 | CG | PHE | A | 117 | 41.251 | 19.926 | 75.734 | 1.00 | 15.32 | A |
| ATOM | 909 | CD1 | PHE | A | 117 | 41.021 | 21.175 | 76.299 | 1.00 | 15.46 | A |
| ATOM | 910 | CD2 | PHE | A | 117 | 40.530 | 19.561 | 74.603 | 1.00 | 15.31 | A |
| ATOM | 911 | CE1 | PHE | A | 117 | 40.089 | 22.049 | 75.743 | 1.00 | 14.43 | A |
| ATOM | 912 | CE2 | PHE | A | 117 | 39.597 | 20.426 | 74.041 | 1.00 | 13.53 | A |
| ATOM | 913 | CZ | PHE | A | 117 | 39.377 | 21.673 | 74.611 | 1.00 | 12.18 | A |
| ATOM | 914 | C | PHE | A | 117 | 44.182 | 20.668 | 76.199 | 1.00 | 16.22 | A |
| ATOM | 915 | O | PHE | A | 117 | 44.557 | 20.882 | 77.352 | 1.00 | 15.07 | A |
| ATOM | 916 | N | PRO | A | 118 | 44.168 | 21.635 | 75.267 | 1.00 | 16.10 | A |
| ATOM | 917 | CD | PRO | A | 118 | 43.841 | 21.506 | 73.836 | 1.00 | 14.40 | A |
| ATOM | 918 | CA | PRO | A | 118 | 44.617 | 22.994 | 75.573 | 1.00 | 14.53 | A |
| ATOM | 919 | CB | PRO | A | 118 | 44.872 | 23.584 | 74.192 | 1.00 | 16.18 | A |
| ATOM | 920 | CG | PRO | A | 118 | 43.798 | 22.954 | 73.379 | 1.00 | 15.51 | A |
| ATOM | 921 | C | PRO | A | 118 | 43.707 | 23.882 | 76.411 | 1.00 | 13.50 | A |
| ATOM | 922 | O | PRO | A | 118 | 42.557 | 24.143 | 76.060 | 1.00 | 13.84 | A |
| ATOM | 923 | N | PHE | A | 119 | 44.254 | 24.342 | 77.525 | 1.00 | 12.96 | A |
| ATOM | 924 | CA | PHE | A | 119 | 43.564 | 25.248 | 78.429 | 1.00 | 11.73 | A |
| ATOM | 925 | CB | PHE | A | 119 | 43.490 | 24.667 | 79.847 | 1.00 | 12.51 | A |
| ATOM | 926 | CG | PHE | A | 119 | 42.357 | 23.708 | 80.067 | 1.00 | 12.99 | A |
| ATOM | 927 | CD1 | PHE | A | 119 | 41.075 | 24.175 | 80.349 | 1.00 | 14.16 | A |
| ATOM | 928 | CD2 | PHE | A | 119 | 42.577 | 22.337 | 80.029 | 1.00 | 14.42 | A |
| ATOM | 929 | CE1 | PHE | A | 119 | 40.029 | 23.285 | 80.594 | 1.00 | 14.48 | A |
| ATOM | 930 | CE2 | PHE | A | 119 | 41.538 | 21.439 | 80.271 | 1.00 | 15.83 | A |
| ATOM | 931 | CZ | PHE | A | 119 | 40.261 | 21.916 | 80.555 | 1.00 | 13.45 | A |
| ATOM | 932 | C | PHE | A | 119 | 44.429 | 26.499 | 78.479 | 1.00 | 9.40 | A |
| ATOM | 933 | O | PHE | A | 119 | 45.655 | 26.406 | 78.432 | 1.00 | 10.17 | A |
| ATOM | 934 | N | VAL | A | 120 | 43.794 | 27.662 | 78.535 | 1.00 | 6.97 | A |
| ATOM | 935 | CA | VAL | A | 120 | 44.512 | 28.925 | 78.673 | 1.00 | 7.33 | A |
| ATOM | 936 | CB | VAL | A | 120 | 44.281 | 29.869 | 77.473 | 1.00 | 6.77 | A |
| ATOM | 937 | CG1 | VAL | A | 120 | 44.914 | 31.229 | 77.752 | 1.00 | 7.12 | A |
| ATOM | 938 | CG2 | VAL | A | 120 | 44.882 | 29.261 | 76.216 | 1.00 | 4.20 | A |
| ATOM | 939 | C | VAL | A | 120 | 43.880 | 29.509 | 79.929 | 1.00 | 6.89 | A |
| ATOM | 940 | O | VAL | A | 120 | 42.659 | 29.630 | 80.009 | 1.00 | 7.98 | A |
| ATOM | 941 | N | ILE | A | 121 | 44.705 | 29.856 | 80.909 | 1.00 | 4.83 | A |
| ATOM | 942 | CA | ILE | A | 121 | 44.207 | 30.373 | 82.180 | 1.00 | 4.93 | A |
| ATOM | 943 | CB | ILE | A | 121 | 44.992 | 29.755 | 83.361 | 1.00 | 4.87 | A |
| ATOM | 944 | CG2 | ILE | A | 121 | 44.326 | 30.134 | 84.690 | 1.00 | 2.73 | A |
| ATOM | 945 | CG1 | ILE | A | 121 | 45.068 | 28.232 | 83.200 | 1.00 | 6.93 | A |
| ATOM | 946 | CD1 | ILE | A | 121 | 43.715 | 27.526 | 83.121 | 1.00 | 9.94 | A |
| ATOM | 947 | C | ILE | A | 121 | 44.292 | 31.890 | 82.280 | 1.00 | 5.76 | A |
| ATOM | 948 | O | ILE | A | 121 | 45.349 | 32.483 | 82.051 | 1.00 | 6.58 | A |
| ATOM | 949 | N | LEU | A | 122 | 43.169 | 32.509 | 82.633 | 1.00 | 6.54 | A |
| ATOM | 950 | CA | LEU | A | 122 | 43.108 | 33.956 | 82.762 | 1.00 | 6.10 | A |
| ATOM | 951 | CB | LEU | A | 122 | 42.007 | 34.521 | 81.852 | 1.00 | 9.88 | A |
| ATOM | 952 | CG | LEU | A | 122 | 42.079 | 34.155 | 80.367 | 1.00 | 12.66 | A |
| ATOM | 953 | CD1 | LEU | A | 122 | 40.900 | 34.780 | 79.622 | 1.00 | 13.01 | A |
| ATOM | 954 | CD2 | LEU | A | 122 | 43.393 | 34.637 | 79.791 | 1.00 | 16.53 | A |
| ATOM | 955 | C | LEU | A | 122 | 42.845 | 34.419 | 84.191 | 1.00 | 7.29 | A |
| ATOM | 956 | O | LEU | A | 122 | 41.900 | 33.968 | 84.840 | 1.00 | 6.50 | A |
| ATOM | 957 | N | GLY | A | 123 | 43.706 | 35.311 | 84.667 | 1.00 | 5.23 | A |
| ATOM | 958 | CA | GLY | A | 123 | 43.558 | 35.902 | 85.980 | 1.00 | 5.23 | A |
| ATOM | 959 | C | GLY | A | 123 | 43.098 | 37.299 | 85.621 | 1.00 | 5.61 | A |
| ATOM | 960 | O | GLY | A | 123 | 43.906 | 38.161 | 85.288 | 1.00 | 7.07 | A |
| ATOM | 961 | N | ASN | A | 124 | 41.789 | 37.512 | 85.668 | 1.00 | 4.05 | A |
| ATOM | 962 | CA | ASN | A | 124 | 41.195 | 38.786 | 85.294 | 1.00 | 5.49 | A |
| ATOM | 963 | CB | ASN | A | 124 | 39.819 | 38.515 | 84.677 | 1.00 | 4.13 | A |
| ATOM | 964 | CG | ASN | A | 124 | 39.263 | 39.709 | 83.934 | 1.00 | 5.41 | A |
| ATOM | 965 | OD1 | ASN | A | 124 | 39.977 | 40.376 | 83.184 | 1.00 | 4.52 | A |
| ATOM | 966 | ND2 | ASN | A | 124 | 37.979 | 39.975 | 84.126 | 1.00 | 4.69 | A |
| ATOM | 967 | C | ASN | A | 124 | 41.083 | 39.817 | 86.416 | 1.00 | 6.89 | A |
| ATOM | 968 | O | ASN | A | 124 | 41.202 | 39.490 | 87.601 | 1.00 | 7.11 | A |
| ATOM | 969 | N | LYS | A | 125 | 40.865 | 41.068 | 86.009 | 1.00 | 5.73 | A |
| ATOM | 970 | CA | LYS | A | 125 | 40.718 | 42.218 | 86.902 | 1.00 | 6.62 | A |
| ATOM | 971 | CB | LYS | A | 125 | 39.681 | 41.921 | 87.991 | 1.00 | 6.64 | A |
| ATOM | 972 | CG | LYS | A | 125 | 38.366 | 41.367 | 87.451 | 1.00 | 5.75 | A |
| ATOM | 973 | CD | LYS | A | 125 | 37.253 | 41.492 | 88.472 | 1.00 | 9.03 | A |
| ATOM | 974 | CE | LYS | A | 125 | 35.953 | 40.898 | 87.936 | 1.00 | 8.19 | A |
| ATOM | 975 | NZ | LYS | A | 125 | 34.843 | 41.012 | 88.922 | 1.00 | 9.91 | A |
| ATOM | 976 | C | LYS | A | 125 | 42.026 | 42.676 | 87.547 | 1.00 | 5.65 | A |
| ATOM | 977 | O | LYS | A | 125 | 42.027 | 43.177 | 88.672 | 1.00 | 7.88 | A |
| ATOM | 978 | N | ILE | A | 126 | 43.137 | 42.530 | 86.831 | 1.00 | 9.57 | A |
| ATOM | 979 | CA | ILE | A | 126 | 44.430 | 42.934 | 87.382 | 1.00 | 10.77 | A |
| ATOM | 980 | CB | ILE | A | 126 | 45.602 | 42.438 | 86.515 | 1.00 | 12.87 | A |
| ATOM | 981 | CG2 | ILE | A | 126 | 45.631 | 40.917 | 86.521 | 1.00 | 16.70 | A |
| ATOM | 982 | CG1 | ILE | A | 126 | 45.476 | 42.995 | 85.097 | 1.00 | 13.50 | A |
| ATOM | 983 | CD1 | ILE | A | 126 | 46.675 | 42.715 | 84.226 | 1.00 | 18.38 | A |
| ATOM | 984 | C | ILE | A | 126 | 44.574 | 44.439 | 87.579 | 1.00 | 10.94 | A |
| ATOM | 985 | O | ILE | A | 126 | 45.569 | 44.901 | 88.139 | 1.00 | 11.80 | A |
| ATOM | 986 | N | ASP | A | 127 | 43.587 | 45.205 | 87.125 | 1.00 | 12.12 | A |
| ATOM | 987 | CA | ASP | A | 127 | 43.621 | 46.651 | 87.299 | 1.00 | 12.66 | A |
| ATOM | 988 | CB | ASP | A | 127 | 42.557 | 47.325 | 86.434 | 1.00 | 10.13 | A |
| ATOM | 989 | CG | ASP | A | 127 | 41.194 | 46.695 | 86.599 | 1.00 | 9.86 | A |
| ATOM | 990 | OD1 | ASP | A | 127 | 40.974 | 45.605 | 86.032 | 1.00 | 8.07 | A |
| ATOM | 991 | OD2 | ASP | A | 127 | 40.347 | 47.281 | 87.307 | 1.00 | 9.79 | A |
| ATOM | 992 | C | ASP | A | 127 | 43.364 | 46.985 | 88.769 | 1.00 | 13.12 | A |
| ATOM | 993 | O | ASP | A | 127 | 43.646 | 48.096 | 89.216 | 1.00 | 13.67 | A |
| ATOM | 994 | N | ILE | A | 128 | 42.819 | 46.020 | 89.509 | 1.00 | 13.94 | A |
| ATOM | 995 | CA | ILE | A | 128 | 42.534 | 46.197 | 90.936 | 1.00 | 14.20 | A |
| ATOM | 996 | CB | ILE | A | 128 | 41.432 | 45.218 | 91.416 | 1.00 | 14.94 | A |
| ATOM | 997 | CG2 | ILE | A | 128 | 41.190 | 45.390 | 92.922 | 1.00 | 14.46 | A |
| ATOM | 998 | CG1 | ILE | A | 128 | 40.138 | 45.477 | 90.641 | 1.00 | 15.26 | A |
| ATOM | 999 | CD1 | ILE | A | 128 | 39.057 | 44.433 | 90.886 | 1.00 | 16.73 | A |
| ATOM | 1000 | C | ILE | A | 128 | 43.808 | 45.942 | 91.739 | 1.00 | 14.37 | A |
| ATOM | 1001 | O | ILE | A | 128 | 44.399 | 44.866 | 91.655 | 1.00 | 14.91 | A |
| ATOM | 1002 | N | SER | A | 129 | 44.221 | 46.935 | 92.519 | 1.00 | 12.93 | A |
| ATOM | 1003 | CA | SER | A | 129 | 45.439 | 46.838 | 93.320 | 1.00 | 14.76 | A |
| ATOM | 1004 | CB | SER | A | 129 | 45.745 | 48.193 | 93.966 | 1.00 | 16.73 | A |
| ATOM | 1005 | OG | SER | A | 129 | 45.975 | 49.186 | 92.982 | 1.00 | 22.27 | A |
| ATOM | 1006 | C | SER | A | 129 | 45.444 | 45.761 | 94.404 | 1.00 | 12.23 | A |
| ATOM | 1007 | O | SER | A | 129 | 46.401 | 44.988 | 94.510 | 1.00 | 13.34 | A |
| ATOM | 1008 | N | GLU | A | 130 | 44.391 | 45.712 | 95.215 | 1.00 | 10.99 | A |
| ATOM | 1009 | CA | GLU | A | 130 | 44.324 | 44.726 | 96.294 | 1.00 | 9.98 | A |
| ATOM | 1010 | CB | GLU | A | 130 | 43.424 | 45.235 | 97.417 | 1.00 | 9.91 | A |
| ATOM | 1011 | CG | GLU | A | 130 | 43.663 | 44.537 | 98.738 | 1.00 | 9.74 | A |
| ATOM | 1012 | CD | GLU | A | 130 | 42.885 | 45.171 | 99.862 | 1.00 | 10.96 | A |
| ATOM | 1013 | OE1 | GLU | A | 130 | 42.900 | 46.418 | 99.952 | 1.00 | 11.06 | A |
| ATOM | 1014 | OE2 | GLU | A | 130 | 42.269 | 44.427 | 100.652 | 1.00 | 11.06 | A |
| ATOM | 1015 | C | GLU | A | 130 | 43.824 | 43.371 | 95.805 | 1.00 | 9.57 | A |
| ATOM | 1016 | O | GLU | A | 130 | 42.634 | 43.193 | 95.537 | 1.00 | 11.77 | A |
| ATOM | 1017 | N | ARG | A | 131 | 44.743 | 42.414 | 95.719 | 1.00 | 9.48 | A |
| ATOM | 1018 | CA | ARG | A | 131 | 44.437 | 41.071 | 95.229 | 1.00 | 9.73 | A |
| ATOM | 1019 | CB | ARG | A | 131 | 45.531 | 40.639 | 94.258 | 1.00 | 10.03 | A |
| ATOM | 1020 | CG | ARG | A | 131 | 45.811 | 41.642 | 93.157 | 1.00 | 12.09 | A |
| ATOM | 1021 | CD | ARG | A | 131 | 47.065 | 41.276 | 92.379 | 1.00 | 15.49 | A |
| ATOM | 1022 | NE | ARG | A | 131 | 47.023 | 39.897 | 91.902 | 1.00 | 17.58 | A |
| ATOM | 1023 | CZ | ARG | A | 131 | 47.738 | 39.433 | 90.881 | 1.00 | 19.52 | A |
| ATOM | 1024 | NH1 | ARG | A | 131 | 48.557 | 40.240 | 90.218 | 1.00 | 19.81 | A |
| ATOM | 1025 | NH2 | ARG | A | 131 | 47.634 | 38.162 | 90.519 | 1.00 | 15.72 | A |
| ATOM | 1026 | C | ARG | A | 131 | 44.306 | 39.998 | 96.303 | 1.00 | 11.52 | A |
| ATOM | 1027 | O | ARG | A | 131 | 44.920 | 40.091 | 97.365 | 1.00 | 11.82 | A |
| ATOM | 1028 | N | GLN | A | 132 | 43.510 | 38.971 | 96.010 | 1.00 | 10.00 | A |
| ATOM | 1029 | CA | GLN | A | 132 | 43.335 | 37.843 | 96.926 | 1.00 | 11.15 | A |
| ATOM | 1030 | CB | GLN | A | 132 | 41.874 | 37.386 | 96.998 | 1.00 | 12.75 | A |
| ATOM | 1031 | CG | GLN | A | 132 | 40.927 | 38.301 | 97.763 | 1.00 | 18.05 | A |
| ATOM | 1032 | CD | GLN | A | 132 | 39.681 | 37.564 | 98.243 | 1.00 | 19.39 | A |
| ATOM | 1033 | OE1 | GLN | A | 132 | 39.108 | 36.748 | 97.518 | 1.00 | 21.08 | A |
| ATOM | 1034 | NE2 | GLN | A | 132 | 39.252 | 37.856 | 99.467 | 1.00 | 20.78 | A |
| ATOM | 1035 | C | GLN | A | 132 | 44.180 | 36.679 | 96.412 | 1.00 | 11.24 | A |
| ATOM | 1036 | O | GLN | A | 132 | 44.443 | 35.721 | 97.141 | 1.00 | 11.90 | A |
| ATOM | 1037 | N | VAL | A | 133 | 44.589 | 36.763 | 95.147 | 1.00 | 9.02 | A |
| ATOM | 1038 | CA | VAL | A | 133 | 45.405 | 35.723 | 94.532 | 1.00 | 8.36 | A |
| ATOM | 1039 | CB | VAL | A | 133 | 44.635 | 34.999 | 93.396 | 1.00 | 7.28 | A |
| ATOM | 1040 | CG1 | VAL | A | 133 | 45.481 | 33.870 | 92.822 | 1.00 | 9.29 | A |
| ATOM | 1041 | CG2 | VAL | A | 133 | 43.314 | 34.460 | 93.924 | 1.00 | 7.33 | A |
| ATOM | 1042 | C | VAL | A | 133 | 46.665 | 36.347 | 93.948 | 1.00 | 9.02 | A |
| ATOM | 1043 | O | VAL | A | 133 | 46.588 | 37.267 | 93.134 | 1.00 | 8.95 | A |
| ATOM | 1044 | N | SER | A | 134 | 47.827 | 35.852 | 94.359 | 1.00 | 9.56 | A |
| ATOM | 1045 | CA | SER | A | 134 | 49.082 | 36.394 | 93.849 | 1.00 | 12.61 | A |
| ATOM | 1046 | CB | SER | A | 134 | 50.215 | 36.142 | 94.844 | 1.00 | 14.97 | A |
| ATOM | 1047 | OG | SER | A | 134 | 50.519 | 34.761 | 94.919 | 1.00 | 17.24 | A |
| ATOM | 1048 | C | SER | A | 134 | 49.461 | 35.786 | 92.499 | 1.00 | 13.23 | A |
| ATOM | 1049 | O | SER | A | 134 | 48.984 | 34.717 | 92.125 | 1.00 | 12.34 | A |
| ATOM | 1050 | N | THR | A | 135 | 50.323 | 36.483 | 91.770 | 1.00 | 13.64 | A |
| ATOM | 1051 | CA | THR | A | 135 | 50.783 | 36.000 | 90.477 | 1.00 | 14.51 | A |
| ATOM | 1052 | CB | THR | A | 135 | 51.746 | 37.007 | 89.820 | 1.00 | 15.52 | A |
| ATOM | 1053 | OG1 | THR | A | 135 | 51.039 | 38.220 | 89.540 | 1.00 | 14.99 | A |
| ATOM | 1054 | CG2 | THR | A | 135 | 52.315 | 36.443 | 88.523 | 1.00 | 15.94 | A |
| ATOM | 1055 | C | THR | A | 135 | 51.516 | 34.684 | 90.712 | 1.00 | 13.63 | A |
| ATOM | 1056 | O | THR | A | 135 | 51.378 | 33.737 | 89.944 | 1.00 | 10.64 | A |
| ATOM | 1057 | N | GLU | A | 136 | 52.285 | 34.632 | 91.793 | 1.00 | 14.58 | A |
| ATOM | 1058 | CA | GLU | A | 136 | 53.037 | 33.431 | 92.133 | 1.00 | 16.54 | A |
| ATOM | 1059 | CB | GLU | A | 136 | 53.855 | 33.670 | 93.403 | 1.00 | 19.73 | A |
| ATOM | 1060 | CG | GLU | A | 136 | 54.666 | 32.467 | 93.837 | 1.00 | 25.53 | A |
| ATOM | 1061 | CD | GLU | A | 136 | 55.689 | 32.057 | 92.796 | 1.00 | 28.78 | A |
| ATOM | 1062 | OE1 | GLU | A | 136 | 56.724 | 32.751 | 92.675 | 1.00 | 31.70 | A |
| ATOM | 1063 | OE2 | GLU | A | 136 | 55.454 | 31.045 | 92.093 | 1.00 | 31.57 | A |
| ATOM | 1064 | C | GLU | A | 136 | 52.131 | 32.219 | 92.340 | 1.00 | 15.24 | A |
| ATOM | 1065 | O | GLU | A | 136 | 52.412 | 31.120 | 91.844 | 1.00 | 13.71 | A |
| ATOM | 1066 | N | GLU | A | 137 | 51.048 | 32.420 | 93.081 | 1.00 | 12.81 | A |
| ATOM | 1067 | CA | GLU | A | 137 | 50.117 | 31.341 | 93.367 | 1.00 | 13.07 | A |
| ATOM | 1068 | CB | GLU | A | 137 | 49.063 | 31.814 | 94.375 | 1.00 | 15.88 | A |
| ATOM | 1069 | CG | GLU | A | 137 | 48.339 | 30.681 | 95.083 | 1.00 | 24.09 | A |
| ATOM | 1070 | CD | GLU | A | 137 | 47.470 | 31.165 | 96.231 | 1.00 | 27.71 | A |
| ATOM | 1071 | OE1 | GLU | A | 137 | 47.969 | 31.947 | 97.070 | 1.00 | 30.04 | A |
| ATOM | 1072 | OE2 | GLU | A | 137 | 46.291 | 30.756 | 96.299 | 1.00 | 31.82 | A |
| ATOM | 1073 | C | GLU | A | 137 | 49.441 | 30.856 | 92.090 | 1.00 | 11.32 | A |
| ATOM | 1074 | O | GLU | A | 137 | 49.234 | 29.655 | 91.905 | 1.00 | 11.46 | A |
| ATOM | 1075 | N | ALA | A | 138 | 49.107 | 31.795 | 91.211 | 1.00 | 9.23 | A |
| ATOM | 1076 | CA | ALA | A | 138 | 48.449 | 31.473 | 89.950 | 1.00 | 10.27 | A |
| ATOM | 1077 | CB | ALA | A | 138 | 47.977 | 32.750 | 89.275 | 1.00 | 10.39 | A |
| ATOM | 1078 | C | ALA | A | 138 | 49.373 | 30.697 | 89.018 | 1.00 | 10.01 | A |
| ATOM | 1079 | O | ALA | A | 138 | 48.958 | 29.727 | 88.391 | 1.00 | 11.98 | A |
| ATOM | 1080 | N | GLN | A | 139 | 50.626 | 31.127 | 88.920 | 1.00 | 12.76 | A |
| ATOM | 1081 | CA | GLN | A | 139 | 51.587 | 30.443 | 88.059 | 1.00 | 11.40 | A |
| ATOM | 1082 | CB | GLN | A | 139 | 52.901 | 31.228 | 88.002 | 1.00 | 14.16 | A |
| ATOM | 1083 | CG | GLN | A | 139 | 52.743 | 32.623 | 87.413 | 1.00 | 16.47 | A |
| ATOM | 1084 | CD | GLN | A | 139 | 54.041 | 33.393 | 87.378 | 1.00 | 17.23 | A |
| ATOM | 1085 | OE1 | GLN | A | 139 | 54.765 | 33.447 | 88.367 | 1.00 | 18.40 | A |
| ATOM | 1086 | NE2 | GLN | A | 139 | 54.335 | 34.009 | 86.239 | 1.00 | 20.67 | A |
| ATOM | 1087 | C | GLN | A | 139 | 51.841 | 29.037 | 88.581 | 1.00 | 11.86 | A |
| ATOM | 1088 | O | GLN | A | 139 | 52.082 | 28.115 | 87.809 | 1.00 | 13.89 | A |
| ATOM | 1089 | N | ALA | A | 140 | 51.784 | 28.877 | 89.899 | 1.00 | 12.78 | A |
| ATOM | 1090 | CA | ALA | A | 140 | 51.999 | 27.575 | 90.518 | 1.00 | 11.25 | A |
| ATOM | 1091 | CB | ALA | A | 140 | 52.050 | 27.714 | 92.038 | 1.00 | 11.78 | A |
| ATOM | 1092 | C | ALA | A | 140 | 50.881 | 26.620 | 90.120 | 1.00 | 11.07 | A |
| ATOM | 1093 | O | ALA | A | 140 | 51.132 | 25.460 | 89.782 | 1.00 | 11.09 | A |
| ATOM | 1094 | N | TRP | A | 141 | 49.643 | 27.099 | 90.154 | 1.00 | 9.67 | A |
| ATOM | 1095 | CA | TRP | A | 141 | 48.534 | 26.238 | 89.789 | 1.00 | 11.30 | A |
| ATOM | 1096 | CB | TRP | A | 141 | 47.187 | 26.933 | 89.998 | 1.00 | 12.23 | A |
| ATOM | 1097 | CG | TRP | A | 141 | 46.042 | 25.975 | 89.800 | 1.00 | 12.97 | A |
| ATOM | 1098 | CD2 | TRP | A | 141 | 45.361 | 25.697 | 88.572 | 1.00 | 11.07 | A |
| ATOM | 1099 | CE2 | TRP | A | 141 | 44.425 | 24.670 | 88.835 | 1.00 | 11.56 | A |
| ATOM | 1100 | CE3 | TRP | A | 141 | 45.453 | 26.210 | 87.273 | 1.00 | 10.03 | A |
| ATOM | 1101 | CD1 | TRP | A | 141 | 45.504 | 25.129 | 90.735 | 1.00 | 13.13 | A |
| ATOM | 1102 | NE1 | TRP | A | 141 | 44.533 | 24.344 | 90.160 | 1.00 | 11.46 | A |
| ATOM | 1103 | CZ2 | TRP | A | 141 | 43.587 | 24.150 | 87.845 | 1.00 | 11.69 | A |
| ATOM | 1104 | CZ3 | TRP | A | 141 | 44.621 | 25.692 | 86.289 | 1.00 | 11.21 | A |
| ATOM | 1105 | CH2 | TRP | A | 141 | 43.700 | 24.673 | 86.582 | 1.00 | 12.72 | A |
| ATOM | 1106 | C | TRP | A | 141 | 48.658 | 25.817 | 88.332 | 1.00 | 9.64 | A |
| ATOM | 1107 | O | TRP | A | 141 | 48.501 | 24.645 | 88.008 | 1.00 | 10.21 | A |
| ATOM | 1108 | N | CYS | A | 142 | 48.938 | 26.776 | 87.454 | 1.00 | 9.77 | A |
| ATOM | 1109 | CA | CYS | A | 142 | 49.067 | 26.475 | 86.038 | 1.00 | 9.24 | A |
| ATOM | 1110 | CB | CYS | A | 142 | 49.308 | 27.760 | 85.238 | 1.00 | 9.78 | A |
| ATOM | 1111 | SG | CYS | A | 142 | 47.909 | 28.915 | 85.261 | 1.00 | 9.50 | A |
| ATOM | 1112 | C | CYS | A | 142 | 50.185 | 25.473 | 85.770 | 1.00 | 11.03 | A |
| ATOM | 1113 | O | CYS | A | 142 | 50.001 | 24.531 | 85.000 | 1.00 | 11.70 | A |
| ATOM | 1114 | N | ARG | A | 143 | 51.339 | 25.663 | 86.405 | 1.00 | 11.62 | A |
| ATOM | 1115 | CA | ARG | A | 143 | 52.458 | 24.740 | 86.201 | 1.00 | 14.52 | A |
| ATOM | 1116 | CB | ARG | A | 143 | 53.734 | 25.254 | 86.884 | 1.00 | 17.59 | A |
| ATOM | 1117 | CG | ARG | A | 143 | 54.398 | 26.450 | 86.222 | 1.00 | 21.91 | A |
| ATOM | 1118 | CD | ARG | A | 143 | 55.835 | 26.602 | 86.721 | 1.00 | 24.48 | A |
| ATOM | 1119 | NE | ARG | A | 143 | 55.900 | 26.663 | 88.178 | 1.00 | 27.05 | A |
| ATOM | 1120 | CZ | ARG | A | 143 | 55.523 | 27.713 | 88.902 | 1.00 | 27.11 | A |
| ATOM | 1121 | NH1 | ARG | A | 143 | 55.058 | 28.802 | 88.306 | 1.00 | 29.55 | A |
| ATOM | 1122 | NH2 | ARG | A | 143 | 55.604 | 27.670 | 90.225 | 1.00 | 27.75 | A |
| ATOM | 1123 | C | ARG | A | 143 | 52.164 | 23.340 | 86.735 | 1.00 | 14.34 | A |
| ATOM | 1124 | O | ARG | A | 143 | 52.572 | 22.341 | 86.140 | 1.00 | 13.45 | A |
| ATOM | 1125 | N | ASP | A | 144 | 51.453 | 23.272 | 87.855 | 1.00 | 14.54 | A |
| ATOM | 1126 | CA | ASP | A | 144 | 51.141 | 21.994 | 88.491 | 1.00 | 14.82 | A |
| ATOM | 1127 | CB | ASP | A | 144 | 50.941 | 22.201 | 89.993 | 1.00 | 15.07 | A |
| ATOM | 1128 | CG | ASP | A | 144 | 52.161 | 22.791 | 90.665 | 1.00 | 18.85 | A |
| ATOM | 1129 | OD1 | ASP | A | 144 | 53.197 | 22.949 | 89.986 | 1.00 | 20.84 | A |
| ATOM | 1130 | OD2 | ASP | A | 144 | 52.087 | 23.096 | 91.874 | 1.00 | 18.27 | A |
| ATOM | 1131 | C | ASP | A | 144 | 49.937 | 21.240 | 87.935 | 1.00 | 15.55 | A |
| ATOM | 1132 | O | ASP | A | 144 | 49.725 | 20.073 | 88.274 | 1.00 | 14.68 | A |
| ATOM | 1133 | N | ASN | A | 145 | 49.150 | 21.890 | 87.086 | 1.00 | 14.18 | A |
| ATOM | 1134 | CA | ASN | A | 145 | 47.972 | 21.236 | 86.539 | 1.00 | 13.71 | A |
| ATOM | 1135 | CB | ASN | A | 145 | 46.706 | 21.941 | 87.038 | 1.00 | 14.63 | A |
| ATOM | 1136 | CG | ASN | A | 145 | 46.514 | 21.788 | 88.532 | 1.00 | 15.94 | A |
| ATOM | 1137 | OD1 | ASN | A | 145 | 47.050 | 22.564 | 89.328 | 1.00 | 17.10 | A |
| ATOM | 1138 | ND2 | ASN | A | 145 | 45.764 | 20.769 | 88.924 | 1.00 | 16.60 | A |
| ATOM | 1139 | C | ASN | A | 145 | 47.929 | 21.115 | 85.026 | 1.00 | 14.42 | A |
| ATOM | 1140 | O | ASN | A | 145 | 46.852 | 21.125 | 84.432 | 1.00 | 15.04 | A |
| ATOM | 1141 | N | GLY | A | 146 | 49.097 | 20.982 | 84.406 | 1.00 | 13.56 | A |
| ATOM | 1142 | CA | GLY | A | 146 | 49.147 | 20.843 | 82.962 | 1.00 | 13.32 | A |
| ATOM | 1143 | C | GLY | A | 146 | 50.090 | 21.828 | 82.306 | 1.00 | 11.99 | A |
| ATOM | 1144 | O | GLY | A | 146 | 50.294 | 21.789 | 81.091 | 1.00 | 11.99 | A |
| ATOM | 1145 | N | ASP | A | 147 | 50.664 | 22.716 | 83.114 | 1.00 | 11.25 | A |
| ATOM | 1146 | CA | ASP | A | 147 | 51.596 | 23.725 | 82.624 | 1.00 | 10.12 | A |
| ATOM | 1147 | CB | ASP | A | 147 | 52.886 | 23.060 | 82.125 | 1.00 | 11.95 | A |
| ATOM | 1148 | CG | ASP | A | 147 | 54.054 | 24.030 | 82.070 | 1.00 | 14.86 | A |
| ATOM | 1149 | OD1 | ASP | A | 147 | 53.947 | 25.124 | 82.670 | 1.00 | 13.09 | A |
| ATOM | 1150 | OD2 | ASP | A | 147 | 55.080 | 23.698 | 81.440 | 1.00 | 15.10 | A |
| ATOM | 1151 | C | ASP | A | 147 | 50.962 | 24.555 | 81.507 | 1.00 | 10.08 | A |
| ATOM | 1152 | O | ASP | A | 147 | 51.530 | 24.715 | 80.429 | 1.00 | 9.48 | A |
| ATOM | 1153 | N | TYR | A | 148 | 49.780 | 25.091 | 81.781 | 1.00 | 7.57 | A |
| ATOM | 1154 | CA | TYR | A | 148 | 49.067 | 25.898 | 80.798 | 1.00 | 8.12 | A |
| ATOM | 1155 | CB | TYR | A | 148 | 47.577 | 25.955 | 81.128 | 1.00 | 9.17 | A |
| ATOM | 1156 | CG | TYR | A | 148 | 46.909 | 24.622 | 81.354 | 1.00 | 8.50 | A |
| ATOM | 1157 | CD1 | TYR | A | 148 | 46.976 | 23.611 | 80.396 | 1.00 | 7.91 | A |
| ATOM | 1158 | CE1 | TYR | A | 148 | 46.315 | 22.398 | 80.585 | 1.00 | 10.29 | A |
| ATOM | 1159 | CD2 | TYR | A | 148 | 46.165 | 24.390 | 82.513 | 1.00 | 9.49 | A |
| ATOM | 1160 | CE2 | TYR | A | 148 | 45.499 | 23.183 | 82.710 | 1.00 | 10.04 | A |
| ATOM | 1161 | CZ | TYR | A | 148 | 45.578 | 22.195 | 81.742 | 1.00 | 10.28 | A |
| ATOM | 1162 | OH | TYR | A | 148 | 44.904 | 21.009 | 81.933 | 1.00 | 12.96 | A |
| ATOM | 1163 | C | TYR | A | 148 | 49.568 | 27.333 | 80.753 | 1.00 | 7.81 | A |
| ATOM | 1164 | O | TYR | A | 148 | 50.152 | 27.833 | 81.720 | 1.00 | 7.86 | A |
| ATOM | 1165 | N | PRO | A | 149 | 49.340 | 28.018 | 79.619 | 1.00 | 7.80 | A |
| ATOM | 1166 | CD | PRO | A | 149 | 48.839 | 27.476 | 78.344 | 1.00 | 8.82 | A |
| ATOM | 1167 | CA | PRO | A | 149 | 49.760 | 29.411 | 79.464 | 1.00 | 8.49 | A |
| ATOM | 1168 | CB | PRO | A | 149 | 49.397 | 29.728 | 78.012 | 1.00 | 9.96 | A |
| ATOM | 1169 | CG | PRO | A | 149 | 49.478 | 28.393 | 77.336 | 1.00 | 9.18 | A |
| ATOM | 1170 | C | PRO | A | 149 | 48.892 | 30.199 | 80.448 | 1.00 | 8.46 | A |
| ATOM | 1171 | O | PRO | A | 149 | 47.720 | 29.861 | 80.651 | 1.00 | 8.03 | A |
| ATOM | 1172 | N | TYR | A | 150 | 49.468 | 31.224 | 81.064 | 1.00 | 5.76 | A |
| ATOM | 1173 | CA | TYR | A | 150 | 48.747 | 32.044 | 82.029 | 1.00 | 7.15 | A |
| ATOM | 1174 | CB | TYR | A | 150 | 49.342 | 31.842 | 83.430 | 1.00 | 6.92 | A |
| ATOM | 1175 | CG | TYR | A | 150 | 48.770 | 32.754 | 84.494 | 1.00 | 8.03 | A |
| ATOM | 1176 | CD1 | TYR | A | 150 | 47.404 | 32.766 | 84.773 | 1.00 | 9.19 | A |
| ATOM | 1177 | CE1 | TYR | A | 150 | 46.874 | 33.617 | 85.748 | 1.00 | 6.26 | A |
| ATOM | 1178 | CD2 | TYR | A | 150 | 49.597 | 33.614 | 85.217 | 1.00 | 7.85 | A |
| ATOM | 1179 | CE2 | TYR | A | 150 | 49.077 | 34.471 | 86.195 | 1.00 | 9.05 | A |
| ATOM | 1180 | CZ | TYR | A | 150 | 47.718 | 34.466 | 86.449 | 1.00 | 8.95 | A |
| ATOM | 1181 | OH | TYR | A | 150 | 47.197 | 35.329 | 87.386 | 1.00 | 7.73 | A |
| ATOM | 1182 | C | TYR | A | 150 | 48.821 | 33.517 | 81.640 | 1.00 | 7.47 | A |
| ATOM | 1183 | O | TYR | A | 150 | 49.892 | 34.032 | 81.303 | 1.00 | 5.24 | A |
| ATOM | 1184 | N | PHE | A | 151 | 47.677 | 34.189 | 81.679 | 1.00 | 5.78 | A |
| ATOM | 1185 | CA | PHE | A | 151 | 47.618 | 35.607 | 81.356 | 1.00 | 5.92 | A |
| ATOM | 1186 | CB | PHE | A | 151 | 46.878 | 35.848 | 80.040 | 1.00 | 7.57 | A |
| ATOM | 1187 | CG | PHE | A | 151 | 47.584 | 35.313 | 78.839 | 1.00 | 7.45 | A |
| ATOM | 1188 | CD1 | PHE | A | 151 | 47.391 | 34.004 | 78.432 | 1.00 | 8.68 | A |
| ATOM | 1189 | CD2 | PHE | A | 151 | 48.454 | 36.125 | 78.115 | 1.00 | 10.12 | A |
| ATOM | 1190 | CE1 | PHE | A | 151 | 48.052 | 33.503 | 77.316 | 1.00 | 11.48 | A |
| ATOM | 1191 | CE2 | PHE | A | 151 | 49.121 | 35.634 | 76.998 | 1.00 | 10.81 | A |
| ATOM | 1192 | CZ | PHE | A | 151 | 48.917 | 34.319 | 76.598 | 1.00 | 10.05 | A |
| ATOM | 1193 | C | PHE | A | 151 | 46.895 | 36.396 | 82.431 | 1.00 | 7.37 | A |
| ATOM | 1194 | O | PHE | A | 151 | 45.806 | 36.010 | 82.870 | 1.00 | 7.51 | A |
| ATOM | 1195 | N | GLU | A | 152 | 47.500 | 37.498 | 82.858 | 1.00 | 7.36 | A |
| ATOM | 1196 | CA | GLU | A | 152 | 46.856 | 38.372 | 83.830 | 1.00 | 7.82 | A |
| ATOM | 1197 | CB | GLU | A | 152 | 47.883 | 39.002 | 84.769 | 1.00 | 10.40 | A |
| ATOM | 1198 | CG | GLU | A | 152 | 48.651 | 37.967 | 85.578 | 1.00 | 13.98 | A |
| ATOM | 1199 | CD | GLU | A | 152 | 48.882 | 38.390 | 87.014 | 1.00 | 14.80 | A |
| ATOM | 1200 | OE1 | GLU | A | 152 | 49.249 | 39.562 | 87.235 | 1.00 | 15.66 | A |
| ATOM | 1201 | OE2 | GLU | A | 152 | 48.703 | 37.546 | 87.920 | 1.00 | 15.61 | A |
| ATOM | 1202 | C | GLU | A | 152 | 46.206 | 39.409 | 82.923 | 1.00 | 6.92 | A |
| ATOM | 1203 | O | GLU | A | 152 | 46.891 | 40.128 | 82.193 | 1.00 | 7.75 | A |
| ATOM | 1204 | N | THR | A | 153 | 44.880 | 39.462 | 82.961 | 1.00 | 6.97 | A |
| ATOM | 1205 | CA | THR | A | 153 | 44.115 | 40.340 | 82.085 | 1.00 | 7.04 | A |
| ATOM | 1206 | CB | THR | A | 153 | 43.171 | 39.517 | 81.211 | 1.00 | 8.18 | A |
| ATOM | 1207 | OG1 | THR | A | 153 | 42.178 | 38.908 | 82.051 | 1.00 | 5.89 | A |
| ATOM | 1208 | CG2 | THR | A | 153 | 43.937 | 38.421 | 80.468 | 1.00 | 9.74 | A |
| ATOM | 1209 | C | THR | A | 153 | 43.230 | 41.349 | 82.788 | 1.00 | 7.44 | A |
| ATOM | 1210 | O | THR | A | 153 | 43.021 | 41.285 | 83.991 | 1.00 | 5.80 | A |
| ATOM | 1211 | N | SER | A | 154 | 42.701 | 42.277 | 81.998 | 1.00 | 7.69 | A |
| ATOM | 1212 | CA | SER | A | 154 | 41.768 | 43.277 | 82.491 | 1.00 | 7.51 | A |
| ATOM | 1213 | CB | SER | A | 154 | 42.474 | 44.525 | 83.017 | 1.00 | 8.86 | A |
| ATOM | 1214 | OG | SER | A | 154 | 41.498 | 45.518 | 83.316 | 1.00 | 7.08 | A |
| ATOM | 1215 | C | SER | A | 154 | 40.852 | 43.680 | 81.355 | 1.00 | 7.26 | A |
| ATOM | 1216 | O | SER | A | 154 | 41.288 | 44.292 | 80.383 | 1.00 | 8.19 | A |
| ATOM | 1217 | N | ALA | A | 155 | 39.580 | 43.317 | 81.465 | 1.00 | 7.09 | A |
| ATOM | 1218 | CA | ALA | A | 155 | 38.618 | 43.700 | 80.439 | 1.00 | 7.00 | A |
| ATOM | 1219 | CB | ALA | A | 155 | 37.297 | 42.971 | 80.658 | 1.00 | 7.62 | A |
| ATOM | 1220 | C | ALA | A | 155 | 38.411 | 45.209 | 80.554 | 1.00 | 7.50 | A |
| ATOM | 1221 | O | ALA | A | 155 | 38.060 | 45.880 | 79.577 | 1.00 | 8.15 | A |
| ATOM | 1222 | N | LYS | A | 156 | 38.651 | 45.740 | 81.751 | 1.00 | 8.82 | A |
| ATOM | 1223 | CA | LYS | A | 156 | 38.466 | 47.165 | 82.007 | 1.00 | 9.17 | A |
| ATOM | 1224 | CB | LYS | A | 156 | 38.548 | 47.457 | 83.508 | 1.00 | 10.17 | A |
| ATOM | 1225 | CG | LYS | A | 156 | 38.179 | 48.896 | 83.854 | 1.00 | 11.64 | A |
| ATOM | 1226 | CD | LYS | A | 156 | 38.113 | 49.120 | 85.352 | 1.00 | 16.49 | A |
| ATOM | 1227 | CE | LYS | A | 156 | 37.722 | 50.560 | 85.672 | 1.00 | 18.09 | A |
| ATOM | 1228 | NZ | LYS | A | 156 | 37.676 | 50.809 | 87.140 | 1.00 | 23.83 | A |
| ATOM | 1229 | C | LYS | A | 156 | 39.449 | 48.056 | 81.255 | 1.00 | 11.35 | A |
| ATOM | 1230 | O | LYS | A | 156 | 39.065 | 49.118 | 80.762 | 1.00 | 10.55 | A |
| ATOM | 1231 | N | ASP | A | 157 | 40.712 | 47.646 | 81.173 | 1.00 | 10.95 | A |
| ATOM | 1232 | CA | ASP | A | 157 | 41.698 | 48.448 | 80.449 | 1.00 | 14.23 | A |
| ATOM | 1233 | CB | ASP | A | 157 | 42.844 | 48.885 | 81.372 | 1.00 | 17.85 | A |
| ATOM | 1234 | CG | ASP | A | 157 | 43.555 | 47.722 | 82.023 | 1.00 | 22.92 | A |
| ATOM | 1235 | OD1 | ASP | A | 157 | 43.741 | 46.683 | 81.361 | 1.00 | 24.46 | A |
| ATOM | 1236 | OD2 | ASP | A | 157 | 43.947 | 47.858 | 83.203 | 1.00 | 29.36 | A |
| ATOM | 1237 | C | ASP | A | 157 | 42.258 | 47.729 | 79.222 | 1.00 | 14.07 | A |
| ATOM | 1238 | O | ASP | A | 157 | 43.146 | 48.250 | 78.540 | 1.00 | 15.61 | A |
| ATOM | 1239 | N | ALA | A | 158 | 41.728 | 46.534 | 78.958 | 1.00 | 11.68 | A |
| ATOM | 1240 | CA | ALA | A | 158 | 42.102 | 45.697 | 77.812 | 1.00 | 11.97 | A |
| ATOM | 1241 | CB | ALA | A | 158 | 42.096 | 46.534 | 76.526 | 1.00 | 12.13 | A |
| ATOM | 1242 | C | ALA | A | 158 | 43.425 | 44.939 | 77.925 | 1.00 | 10.12 | A |
| ATOM | 1243 | O | ALA | A | 158 | 43.804 | 44.211 | 77.007 | 1.00 | 10.95 | A |
| ATOM | 1244 | N | THR | A | 159 | 44.116 | 45.095 | 79.048 | 1.00 | 9.83 | A |
| ATOM | 1245 | CA | THR | A | 159 | 45.394 | 44.426 | 79.257 | 1.00 | 9.64 | A |
| ATOM | 1246 | CB | THR | A | 159 | 45.888 | 44.612 | 80.707 | 1.00 | 12.74 | A |
| ATOM | 1247 | OG1 | THR | A | 159 | 45.999 | 46.008 | 81.005 | 1.00 | 14.77 | A |
| ATOM | 1248 | CG2 | THR | A | 159 | 47.246 | 43.965 | 80.892 | 1.00 | 13.03 | A |
| ATOM | 1249 | C | THR | A | 159 | 45.350 | 42.926 | 78.956 | 1.00 | 10.27 | A |
| ATOM | 1250 | O | THR | A | 159 | 44.540 | 42.191 | 79.523 | 1.00 | 8.82 | A |
| ATOM | 1251 | N | ASN | A | 160 | 46.223 | 42.497 | 78.048 | 1.00 | 8.41 | A |
| ATOM | 1252 | CA | ASN | A | 160 | 46.365 | 41.095 | 77.648 | 1.00 | 8.76 | A |
| ATOM | 1253 | CB | ASN | A | 160 | 46.976 | 40.276 | 78.789 | 1.00 | 9.61 | A |
| ATOM | 1254 | CG | ASN | A | 160 | 48.440 | 40.573 | 78.993 | 1.00 | 14.25 | A |
| ATOM | 1255 | OD1 | ASN | A | 160 | 49.178 | 40.760 | 78.029 | 1.00 | 13.62 | A |
| ATOM | 1256 | ND2 | ASN | A | 160 | 48.875 | 40.606 | 80.249 | 1.00 | 12.27 | A |
| ATOM | 1257 | C | ASN | A | 160 | 45.141 | 40.351 | 77.132 | 1.00 | 7.94 | A |
| ATOM | 1258 | O | ASN | A | 160 | 45.182 | 39.128 | 77.001 | 1.00 | 7.38 | A |
| ATOM | 1259 | N | VAL | A | 161 | 44.062 | 41.058 | 76.832 | 1.00 | 6.94 | A |
| ATOM | 1260 | CA | VAL | A | 161 | 42.872 | 40.380 | 76.326 | 1.00 | 6.49 | A |
| ATOM | 1261 | CB | VAL | A | 161 | 41.669 | 41.337 | 76.259 | 1.00 | 7.13 | A |
| ATOM | 1262 | CG1 | VAL | A | 161 | 40.463 | 40.625 | 75.638 | 1.00 | 8.90 | A |
| ATOM | 1263 | CG2 | VAL | A | 161 | 41.332 | 41.819 | 77.664 | 1.00 | 7.05 | A |
| ATOM | 1264 | C | VAL | A | 161 | 43.122 | 39.759 | 74.945 | 1.00 | 6.57 | A |
| ATOM | 1265 | O | VAL | A | 161 | 42.907 | 38.564 | 74.760 | 1.00 | 7.56 | A |
| ATOM | 1266 | N | ALA | A | 162 | 43.580 | 40.556 | 73.981 | 1.00 | 5.01 | A |
| ATOM | 1267 | CA | ALA | A | 162 | 43.851 | 40.029 | 72.642 | 1.00 | 6.79 | A |
| ATOM | 1268 | CB | ALA | A | 162 | 44.281 | 41.153 | 71.702 | 1.00 | 6.69 | A |
| ATOM | 1269 | C | ALA | A | 162 | 44.930 | 38.944 | 72.685 | 1.00 | 6.82 | A |
| ATOM | 1270 | O | ALA | A | 162 | 44.856 | 37.955 | 71.955 | 1.00 | 5.96 | A |
| ATOM | 1271 | N | ALA | A | 163 | 45.930 | 39.131 | 73.542 | 1.00 | 7.06 | A |
| ATOM | 1272 | CA | ALA | A | 163 | 47.015 | 38.160 | 73.674 | 1.00 | 7.58 | A |
| ATOM | 1273 | CB | ALA | A | 163 | 48.070 | 38.676 | 74.652 | 1.00 | 6.60 | A |
| ATOM | 1274 | C | ALA | A | 163 | 46.512 | 36.793 | 74.137 | 1.00 | 7.05 | A |
| ATOM | 1275 | O | ALA | A | 163 | 46.952 | 35.761 | 73.632 | 1.00 | 7.32 | A |
| ATOM | 1276 | N | ALA | A | 164 | 45.595 | 36.787 | 75.099 | 1.00 | 5.16 | A |
| ATOM | 1277 | CA | ALA | A | 164 | 45.053 | 35.532 | 75.613 | 1.00 | 6.05 | A |
| ATOM | 1278 | CB | ALA | A | 164 | 44.165 | 35.802 | 76.817 | 1.00 | 7.19 | A |
| ATOM | 1279 | C | ALA | A | 164 | 44.268 | 34.787 | 74.538 | 1.00 | 7.48 | A |
| ATOM | 1280 | O | ALA | A | 164 | 44.441 | 33.585 | 74.354 | 1.00 | 7.36 | A |
| ATOM | 1281 | N | PHE | A | 165 | 43.402 | 35.501 | 73.827 | 1.00 | 7.41 | A |
| ATOM | 1282 | CA | PHE | A | 165 | 42.612 | 34.875 | 72.770 | 1.00 | 8.84 | A |
| ATOM | 1283 | CB | PHE | A | 165 | 41.591 | 35.868 | 72.213 | 1.00 | 9.11 | A |
| ATOM | 1284 | CG | PHE | A | 165 | 40.340 | 35.973 | 73.038 | 1.00 | 10.05 | A |
| ATOM | 1285 | CD1 | PHE | A | 165 | 39.394 | 34.952 | 73.019 | 1.00 | 10.31 | A |
| ATOM | 1286 | CD2 | PHE | A | 165 | 40.113 | 37.082 | 73.847 | 1.00 | 8.40 | A |
| ATOM | 1287 | CE1 | PHE | A | 165 | 38.237 | 35.038 | 73.797 | 1.00 | 10.67 | A |
| ATOM | 1288 | CE2 | PHE | A | 165 | 38.961 | 37.176 | 74.628 | 1.00 | 11.52 | A |
| ATOM | 1289 | CZ | PHE | A | 165 | 38.023 | 36.152 | 74.601 | 1.00 | 9.30 | A |
| ATOM | 1290 | C | PHE | A | 165 | 43.506 | 34.349 | 71.656 | 1.00 | 10.24 | A |
| ATOM | 1291 | O | PHE | A | 165 | 43.268 | 33.263 | 71.125 | 1.00 | 10.76 | A |
| ATOM | 1292 | N | GLU | A | 166 | 44.540 | 35.116 | 71.313 | 1.00 | 9.97 | A |
| ATOM | 1293 | CA | GLU | A | 166 | 45.483 | 34.720 | 70.273 | 1.00 | 9.74 | A |
| ATOM | 1294 | CB | GLU | A | 166 | 46.448 | 35.876 | 69.989 | 1.00 | 10.73 | A |
| ATOM | 1295 | CG | GLU | A | 166 | 45.832 | 36.963 | 69.113 | 1.00 | 11.76 | A |
| ATOM | 1296 | CD | GLU | A | 166 | 46.454 | 38.332 | 69.327 | 1.00 | 13.89 | A |
| ATOM | 1297 | OE1 | GLU | A | 166 | 47.545 | 38.414 | 69.931 | 1.00 | 14.52 | A |
| ATOM | 1298 | OE2 | GLU | A | 166 | 45.850 | 39.334 | 68.881 | 1.00 | 13.38 | A |
| ATOM | 1299 | C | GLU | A | 166 | 46.255 | 33.458 | 70.670 | 1.00 | 10.36 | A |
| ATOM | 1300 | O | GLU | A | 166 | 46.555 | 32.610 | 69.829 | 1.00 | 9.15 | A |
| ATOM | 1301 | N | GLU | A | 167 | 46.569 | 33.336 | 71.956 | 1.00 | 10.04 | A |
| ATOM | 1302 | CA | GLU | A | 167 | 47.288 | 32.170 | 72.461 | 1.00 | 10.64 | A |
| ATOM | 1303 | CB | GLU | A | 167 | 47.650 | 32.370 | 73.938 | 1.00 | 12.43 | A |
| ATOM | 1304 | CG | GLU | A | 167 | 48.392 | 31.197 | 74.591 | 1.00 | 14.29 | A |
| ATOM | 1305 | CD | GLU | A | 167 | 49.685 | 30.837 | 73.879 | 1.00 | 16.86 | A |
| ATOM | 1306 | OE1 | GLU | A | 167 | 50.393 | 31.756 | 73.417 | 1.00 | 18.15 | A |
| ATOM | 1307 | OE2 | GLU | A | 167 | 50.002 | 29.632 | 73.792 | 1.00 | 18.43 | A |
| ATOM | 1308 | C | GLU | A | 167 | 46.408 | 30.935 | 72.301 | 1.00 | 8.99 | A |
| ATOM | 1309 | O | GLU | A | 167 | 46.902 | 29.843 | 72.024 | 1.00 | 9.52 | A |
| ATOM | 1310 | N | ALA | A | 168 | 45.102 | 31.110 | 72.485 | 1.00 | 9.67 | A |
| ATOM | 1311 | CA | ALA | A | 168 | 44.172 | 29.999 | 72.328 | 1.00 | 8.43 | A |
| ATOM | 1312 | CB | ALA | A | 168 | 42.732 | 30.475 | 72.532 | 1.00 | 10.29 | A |
| ATOM | 1313 | C | ALA | A | 168 | 44.341 | 29.420 | 70.928 | 1.00 | 8.01 | A |
| ATOM | 1314 | O | ALA | A | 168 | 44.396 | 28.200 | 70.753 | 1.00 | 6.98 | A |
| ATOM | 1315 | N | VAL | A | 169 | 44.433 | 30.292 | 69.925 | 1.00 | 7.90 | A |
| ATOM | 1316 | CA | VAL | A | 169 | 44.603 | 29.811 | 68.563 | 1.00 | 7.99 | A |
| ATOM | 1317 | CB | VAL | A | 169 | 44.541 | 30.953 | 67.525 | 1.00 | 7.93 | A |
| ATOM | 1318 | CG1 | VAL | A | 169 | 44.605 | 30.365 | 66.133 | 1.00 | 9.88 | A |
| ATOM | 1319 | CG2 | VAL | A | 169 | 43.256 | 31.740 | 67.684 | 1.00 | 8.52 | A |
| ATOM | 1320 | C | VAL | A | 169 | 45.925 | 29.066 | 68.395 | 1.00 | 8.88 | A |
| ATOM | 1321 | O | VAL | A | 169 | 45.962 | 28.014 | 67.756 | 1.00 | 8.16 | A |
| ATOM | 1322 | N | ARG | A | 170 | 47.008 | 29.601 | 68.963 | 1.00 | 8.25 | A |
| ATOM | 1323 | CA | ARG | A | 170 | 48.306 | 28.932 | 68.868 | 1.00 | 9.31 | A |
| ATOM | 1324 | CB | ARG | A | 170 | 49.395 | 29.722 | 69.601 | 1.00 | 10.10 | A |
| ATOM | 1325 | CG | ARG | A | 170 | 49.845 | 30.991 | 68.907 | 1.00 | 10.42 | A |
| ATOM | 1326 | CD | ARG | A | 170 | 51.003 | 31.648 | 69.668 | 1.00 | 11.49 | A |
| ATOM | 1327 | NE | ARG | A | 170 | 51.387 | 32.939 | 69.091 | 1.00 | 10.32 | A |
| ATOM | 1328 | CZ | ARG | A | 170 | 52.136 | 33.092 | 68.000 | 1.00 | 13.81 | A |
| ATOM | 1329 | NH1 | ARG | A | 170 | 52.603 | 32.033 | 67.346 | 1.00 | 12.47 | A |
| ATOM | 1330 | NH2 | ARG | A | 170 | 52.415 | 34.313 | 67.557 | 1.00 | 12.77 | A |
| ATOM | 1331 | C | ARG | A | 170 | 48.226 | 27.535 | 69.482 | 1.00 | 9.33 | A |
| ATOM | 1332 | O | ARG | A | 170 | 48.797 | 26.581 | 68.951 | 1.00 | 10.73 | A |
| ATOM | 1333 | N | ARG | A | 171 | 47.519 | 27.422 | 70.603 | 1.00 | 8.82 | A |
| ATOM | 1334 | CA | ARG | A | 171 | 47.375 | 26.138 | 71.281 | 1.00 | 9.95 | A |
| ATOM | 1335 | CB | ARG | A | 171 | 46.713 | 26.331 | 72.649 | 1.00 | 11.53 | A |
| ATOM | 1336 | CG | ARG | A | 171 | 47.592 | 27.085 | 73.649 | 1.00 | 12.66 | A |
| ATOM | 1337 | CD | ARG | A | 171 | 48.930 | 26.372 | 73.841 | 1.00 | 16.04 | A |
| ATOM | 1338 | NE | ARG | A | 171 | 48.755 | 25.042 | 74.418 | 1.00 | 18.94 | A |
| ATOM | 1339 | CZ | ARG | A | 171 | 49.531 | 23.994 | 74.149 | 1.00 | 21.84 | A |
| ATOM | 1340 | NH1 | ARG | A | 171 | 50.549 | 24.109 | 73.301 | 1.00 | 20.29 | A |
| ATOM | 1341 | NH2 | ARG | A | 171 | 49.289 | 22.828 | 74.733 | 1.00 | 22.16 | A |
| ATOM | 1342 | C | ARG | A | 171 | 46.577 | 25.139 | 70.442 | 1.00 | 10.95 | A |
| ATOM | 1343 | O | ARG | A | 171 | 46.891 | 23.950 | 70.421 | 1.00 | 9.94 | A |
| ATOM | 1344 | N | VAL | A | 172 | 45.548 | 25.616 | 69.748 | 1.00 | 9.08 | A |
| ATOM | 1345 | CA | VAL | A | 172 | 44.760 | 24.720 | 68.913 | 1.00 | 10.59 | A |
| ATOM | 1346 | CB | VAL | A | 172 | 43.484 | 25.410 | 68.379 | 1.00 | 7.87 | A |
| ATOM | 1347 | CG1 | VAL | A | 172 | 42.750 | 24.487 | 67.393 | 1.00 | 9.83 | A |
| ATOM | 1348 | CG2 | VAL | A | 172 | 42.571 | 25.762 | 69.538 | 1.00 | 8.00 | A |
| ATOM | 1349 | C | VAL | A | 172 | 45.618 | 24.256 | 67.740 | 1.00 | 11.82 | A |
| ATOM | 1350 | O | VAL | A | 172 | 45.604 | 23.080 | 67.374 | 1.00 | 13.22 | A |
| ATOM | 1351 | N | LEU | A | 173 | 46.380 | 25.178 | 67.163 | 1.00 | 12.82 | A |
| ATOM | 1352 | CA | LEU | A | 173 | 47.238 | 24.844 | 66.032 | 1.00 | 14.06 | A |
| ATOM | 1353 | CB | LEU | A | 173 | 47.946 | 26.099 | 65.512 | 1.00 | 13.05 | A |
| ATOM | 1354 | CG | LEU | A | 173 | 47.072 | 27.158 | 64.836 | 1.00 | 13.09 | A |
| ATOM | 1355 | CD1 | LEU | A | 173 | 47.922 | 28.383 | 64.532 | 1.00 | 14.62 | A |
| ATOM | 1356 | CD2 | LEU | A | 173 | 46.462 | 26.607 | 63.552 | 1.00 | 15.93 | A |
| ATOM | 1357 | C | LEU | A | 173 | 48.271 | 23.787 | 66.412 | 1.00 | 15.53 | A |
| ATOM | 1358 | O | LEU | A | 173 | 48.625 | 22.939 | 65.595 | 1.00 | 15.94 | A |
| ATOM | 1359 | N | ALA | A | 174 | 48.746 | 23.833 | 67.652 | 1.00 | 15.74 | A |
| ATOM | 1360 | CA | ALA | A | 174 | 49.734 | 22.867 | 68.125 | 1.00 | 18.63 | A |
| ATOM | 1361 | CB | ALA | A | 174 | 50.421 | 23.392 | 69.393 | 1.00 | 16.69 | A |
| ATOM | 1362 | C | ALA | A | 174 | 49.055 | 21.529 | 68.404 | 1.00 | 21.61 | A |
| ATOM | 1363 | O | ALA | A | 174 | 49.699 | 20.477 | 68.414 | 1.00 | 21.44 | A |
| ATOM | 1364 | N | THR | A | 175 | 47.746 | 21.583 | 68.628 | 1.00 | 23.37 | A |
| ATOM | 1365 | CA | THR | A | 175 | 46.950 | 20.393 | 68.900 | 1.00 | 26.99 | A |
| ATOM | 1366 | CB | THR | A | 175 | 45.855 | 20.683 | 69.953 | 1.00 | 26.86 | A |
| ATOM | 1367 | OG1 | THR | A | 175 | 46.456 | 21.224 | 71.136 | 1.00 | 29.85 | A |
| ATOM | 1368 | CG2 | THR | A | 175 | 45.115 | 19.406 | 70.318 | 1.00 | 28.99 | A |
| ATOM | 1369 | C | THR | A | 175 | 46.284 | 19.927 | 67.609 | 1.00 | 28.49 | A |
| ATOM | 1370 | O | THR | A | 175 | 45.075 | 20.196 | 67.442 | 1.00 | 30.38 | A |
| ATOM | 1371 | OXT | THR | A | 175 | 46.983 | 19.319 | 66.768 | 1.00 | 31.89 | A |
| TER | | | | | | | | | | | |
| ATOM | 1372 | CB | LYS | B | 4 | 10.515 | 14.129 | 75.392 | 1.00 | 39.31 | B |
| ATOM | 1373 | CG | LYS | B | 4 | 10.185 | 15.485 | 74.760 | 1.00 | 40.02 | B |
| ATOM | 1374 | CD | LYS | B | 4 | 11.398 | 16.404 | 74.709 | 1.00 | 40.45 | B |
| ATOM | 1375 | CE | LYS | B | 4 | 11.122 | 17.646 | 73.877 | 1.00 | 42.05 | B |
| ATOM | 1376 | NZ | LYS | B | 4 | 10.858 | 17.321 | 72.449 | 1.00 | 42.02 | B |
| ATOM | 1377 | C | LYS | B | 4 | 12.927 | 13.480 | 75.210 | 1.00 | 36.91 | B |
| ATOM | 1378 | O | LYS | B | 4 | 13.403 | 12.637 | 75.969 | 1.00 | 37.82 | B |
| ATOM | 1379 | N | LYS | B | 4 | 11.169 | 11.850 | 74.674 | 1.00 | 39.43 | B |
| ATOM | 1380 | CA | LYS | B | 4 | 11.537 | 13.293 | 74.611 | 1.00 | 37.68 | B |
| ATOM | 1381 | N | SER | B | 5 | 13.581 | 14.579 | 74.853 | 1.00 | 34.24 | B |
| ATOM | 1382 | CA | SER | B | 5 | 14.909 | 14.876 | 75.374 | 1.00 | 31.72 | B |
| ATOM | 1383 | CB | SER | B | 5 | 15.738 | 15.617 | 74.325 | 1.00 | 32.47 | B |
| ATOM | 1384 | OG | SER | B | 5 | 15.101 | 16.824 | 73.943 | 1.00 | 32.57 | B |
| ATOM | 1385 | C | SER | B | 5 | 14.741 | 15.752 | 76.608 | 1.00 | 29.67 | B |
| ATOM | 1386 | O | SER | B | 5 | 15.719 | 16.242 | 77.172 | 1.00 | 29.16 | B |
| ATOM | 1387 | N | SER | B | 6 | 13.487 | 15.935 | 77.013 | 1.00 | 26.94 | B |
| ATOM | 1388 | CA | SER | B | 6 | 13.138 | 16.753 | 78.170 | 1.00 | 25.14 | B |
| ATOM | 1389 | CB | SER | B | 6 | 13.765 | 16.176 | 79.440 | 1.00 | 24.53 | B |
| ATOM | 1390 | OG | SER | B | 6 | 13.199 | 14.910 | 79.745 | 1.00 | 25.36 | B |
| ATOM | 1391 | C | SER | B | 6 | 13.587 | 18.195 | 77.964 | 1.00 | 23.83 | B |
| ATOM | 1392 | O | SER | B | 6 | 13.901 | 18.911 | 78.915 | 1.00 | 25.73 | B |
| ATOM | 1393 | N | LEU | B | 7 | 13.608 | 18.612 | 76.707 | 1.00 | 21.20 | B |
| ATOM | 1394 | CA | LEU | B | 7 | 14.011 | 19.964 | 76.354 | 1.00 | 18.14 | B |
| ATOM | 1395 | CB | LEU | B | 7 | 15.533 | 20.029 | 76.211 | 1.00 | 21.24 | B |
| ATOM | 1396 | CG | LEU | B | 7 | 16.211 | 21.397 | 76.190 | 1.00 | 22.95 | B |
| ATOM | 1397 | CD1 | LEU | B | 7 | 15.947 | 22.126 | 77.500 | 1.00 | 21.85 | B |
| ATOM | 1398 | CD2 | LEU | B | 7 | 17.711 | 21.207 | 75.984 | 1.00 | 24.71 | B |
| ATOM | 1399 | C | LEU | B | 7 | 13.345 | 20.302 | 75.028 | 1.00 | 14.83 | B |
| ATOM | 1400 | O | LEU | B | 7 | 13.533 | 19.592 | 74.039 | 1.00 | 14.50 | B |
| ATOM | 1401 | N | PHE | B | 8 | 12.548 | 21.365 | 75.016 | 1.00 | 10.36 | B |
| ATOM | 1402 | CA | PHE | B | 8 | 11.866 | 21.787 | 73.798 | 1.00 | 8.45 | B |
| ATOM | 1403 | CB | PHE | B | 8 | 10.426 | 22.220 | 74.101 | 1.00 | 7.43 | B |
| ATOM | 1404 | CG | PHE | B | 8 | 9.507 | 21.077 | 74.459 | 1.00 | 9.64 | B |
| ATOM | 1405 | CD1 | PHE | B | 8 | 9.701 | 20.345 | 75.625 | 1.00 | 11.29 | B |
| ATOM | 1406 | CD2 | PHE | B | 8 | 8.444 | 20.740 | 73.627 | 1.00 | 10.54 | B |
| ATOM | 1407 | CE1 | PHE | B | 8 | 8.846 | 19.292 | 75.958 | 1.00 | 12.93 | B |
| ATOM | 1408 | CE2 | PHE | B | 8 | 7.586 | 19.690 | 73.951 | 1.00 | 10.69 | B |
| ATOM | 1409 | CZ | PHE | B | 8 | 7.789 | 18.966 | 75.118 | 1.00 | 11.50 | B |
| ATOM | 1410 | C | PHE | B | 8 | 12.637 | 22.950 | 73.194 | 1.00 | 7.21 | B |
| ATOM | 1411 | O | PHE | B | 8 | 12.540 | 24.075 | 73.667 | 1.00 | 7.86 | B |
| ATOM | 1412 | N | LYS | B | 9 | 13.411 | 22.665 | 72.153 | 1.00 | 7.12 | B |
| ATOM | 1413 | CA | LYS | B | 9 | 14.210 | 23.690 | 71.496 | 1.00 | 5.21 | B |
| ATOM | 1414 | CB | LYS | B | 9 | 15.391 | 23.041 | 70.774 | 1.00 | 4.04 | B |
| ATOM | 1415 | CG | LYS | B | 9 | 16.258 | 24.028 | 70.000 | 1.00 | 3.99 | B |
| ATOM | 1416 | CD | LYS | B | 9 | 17.480 | 23.332 | 69.428 | 1.00 | 5.86 | B |
| ATOM | 1417 | CE | LYS | B | 9 | 18.356 | 24.302 | 68.642 | 1.00 | 7.04 | B |
| ATOM | 1418 | NZ | LYS | B | 9 | 19.543 | 23.607 | 68.076 | 1.00 | 8.71 | B |
| ATOM | 1419 | C | LYS | B | 9 | 13.369 | 24.493 | 70.513 | 1.00 | 4.76 | B |
| ATOM | 1420 | O | LYS | B | 9 | 12.856 | 23.959 | 69.526 | 1.00 | 5.88 | B |
| ATOM | 1421 | N | VAL | B | 10 | 13.233 | 25.784 | 70.797 | 1.00 | 6.25 | B |
| ATOM | 1422 | CA | VAL | B | 10 | 12.455 | 26.683 | 69.960 | 1.00 | 7.54 | B |
| ATOM | 1423 | CB | VAL | B | 10 | 11.292 | 27.294 | 70.766 | 1.00 | 7.75 | B |
| ATOM | 1424 | CG1 | VAL | B | 10 | 10.471 | 28.220 | 69.888 | 1.00 | 9.02 | B |
| ATOM | 1425 | CG2 | VAL | B | 10 | 10.412 | 26.168 | 71.318 | 1.00 | 11.39 | B |
| ATOM | 1426 | C | VAL | B | 10 | 13.388 | 27.778 | 69.454 | 1.00 | 7.20 | B |
| ATOM | 1427 | O | VAL | B | 10 | 14.019 | 28.480 | 70.246 | 1.00 | 9.06 | B |
| ATOM | 1428 | N | ILE | B | 11 | 13.468 | 27.917 | 68.134 | 1.00 | 6.43 | B |
| ATOM | 1429 | CA | ILE | B | 11 | 14.362 | 28.892 | 67.525 | 1.00 | 7.00 | B |
| ATOM | 1430 | CB | ILE | B | 11 | 15.294 | 28.191 | 66.498 | 1.00 | 7.60 | B |
| ATOM | 1431 | CG2 | ILE | B | 11 | 14.475 | 27.609 | 65.351 | 1.00 | 7.21 | B |
| ATOM | 1432 | CG1 | ILE | B | 11 | 16.334 | 29.174 | 65.964 | 1.00 | 10.00 | B |
| ATOM | 1433 | CD1 | ILE | B | 11 | 17.448 | 28.493 | 65.183 | 1.00 | 14.68 | B |
| ATOM | 1434 | C | ILE | B | 11 | 13.673 | 30.077 | 66.861 | 1.00 | 6.59 | B |
| ATOM | 1435 | O | ILE | B | 11 | 12.688 | 29.918 | 66.134 | 1.00 | 6.04 | B |
| ATOM | 1436 | N | LEU | B | 12 | 14.200 | 31.268 | 67.132 | 1.00 | 5.58 | B |
| ATOM | 1437 | CA | LEU | B | 12 | 13.669 | 32.509 | 66.564 | 1.00 | 7.26 | B |
| ATOM | 1438 | CB | LEU | B | 12 | 13.836 | 33.661 | 67.561 | 1.00 | 9.82 | B |
| ATOM | 1439 | CG | LEU | B | 12 | 12.914 | 33.692 | 68.783 | 1.00 | 13.45 | B |
| ATOM | 1440 | CD1 | LEU | B | 12 | 13.533 | 34.548 | 69.885 | 1.00 | 13.54 | B |
| ATOM | 1441 | CD2 | LEU | B | 12 | 11.553 | 34.237 | 68.364 | 1.00 | 12.76 | B |
| ATOM | 1442 | C | LEU | B | 12 | 14.410 | 32.855 | 65.274 | 1.00 | 7.87 | B |
| ATOM | 1443 | O | LEU | B | 12 | 15.632 | 33.018 | 65.282 | 1.00 | 7.19 | B |
| ATOM | 1444 | N | LEU | B | 13 | 13.670 | 32.957 | 64.172 | 1.00 | 6.70 | B |
| ATOM | 1445 | CA | LEU | B | 13 | 14.247 | 33.313 | 62.875 | 1.00 | 7.53 | B |
| ATOM | 1446 | CB | LEU | B | 13 | 14.192 | 32.136 | 61.891 | 1.00 | 7.41 | B |
| ATOM | 1447 | CG | LEU | B | 13 | 15.003 | 30.875 | 62.209 | 1.00 | 7.60 | B |
| ATOM | 1448 | CD1 | LEU | B | 13 | 14.848 | 29.885 | 61.062 | 1.00 | 10.29 | B |
| ATOM | 1449 | CD2 | LEU | B | 13 | 16.468 | 31.222 | 62.416 | 1.00 | 8.47 | B |
| ATOM | 1450 | C | LEU | B | 13 | 13.445 | 34.472 | 62.303 | 1.00 | 5.47 | B |
| ATOM | 1451 | O | LEU | B | 13 | 12.252 | 34.594 | 62.574 | 1.00 | 6.51 | B |
| ATOM | 1452 | N | GLY | B | 14 | 14.099 | 35.312 | 61.508 | 1.00 | 6.46 | B |
| ATOM | 1453 | CA | GLY | B | 14 | 13.424 | 36.450 | 60.910 | 1.00 | 5.61 | B |
| ATOM | 1454 | C | GLY | B | 14 | 14.401 | 37.565 | 60.595 | 1.00 | 7.62 | B |
| ATOM | 1455 | O | GLY | B | 14 | 15.484 | 37.623 | 61.177 | 1.00 | 7.00 | B |
| ATOM | 1456 | N | ASP | B | 15 | 14.023 | 38.451 | 59.676 | 1.00 | 8.24 | B |
| ATOM | 1457 | CA | ASP | B | 15 | 14.886 | 39.563 | 59.272 | 1.00 | 7.19 | B |
| ATOM | 1458 | CB | ASP | B | 15 | 14.127 | 40.521 | 58.356 | 1.00 | 7.69 | B |
| ATOM | 1459 | CG | ASP | B | 15 | 13.982 | 39.998 | 56.944 | 1.00 | 9.45 | B |
| ATOM | 1460 | OD1 | ASP | B | 15 | 14.333 | 38.829 | 56.690 | 1.00 | 8.96 | B |
| ATOM | 1461 | OD2 | ASP | B | 15 | 13.506 | 40.772 | 56.084 | 1.00 | 10.34 | B |
| ATOM | 1462 | C | ASP | B | 15 | 15.449 | 40.373 | 60.431 | 1.00 | 7.23 | B |
| ATOM | 1463 | O | ASP | B | 15 | 14.858 | 40.445 | 61.504 | 1.00 | 7.84 | B |
| ATOM | 1464 | N | GLY | B | 16 | 16.595 | 41.003 | 60.201 | 1.00 | 8.03 | B |
| ATOM | 1465 | CA | GLY | B | 16 | 17.181 | 41.819 | 61.245 | 1.00 | 8.90 | B |
| ATOM | 1466 | C | GLY | B | 16 | 16.246 | 42.967 | 61.583 | 1.00 | 9.61 | B |
| ATOM | 1467 | O | GLY | B | 16 | 15.622 | 43.542 | 60.691 | 1.00 | 12.19 | B |
| ATOM | 1468 | N | GLY | B | 17 | 16.122 | 43.287 | 62.868 | 1.00 | 8.45 | B |
| ATOM | 1469 | CA | GLY | B | 17 | 15.272 | 44.394 | 63.275 | 1.00 | 7.78 | B |
| ATOM | 1470 | C | GLY | B | 17 | 13.799 | 44.121 | 63.529 | 1.00 | 8.07 | B |
| ATOM | 1471 | O | GLY | B | 17 | 13.085 | 45.020 | 63.987 | 1.00 | 7.90 | B |
| ATOM | 1472 | N | VAL | B | 18 | 13.327 | 42.904 | 63.253 | 1.00 | 6.46 | B |
| ATOM | 1473 | CA | VAL | B | 18 | 11.911 | 42.601 | 63.468 | 1.00 | 6.02 | B |
| ATOM | 1474 | CB | VAL | B | 18 | 11.481 | 41.294 | 62.744 | 1.00 | 4.56 | B |
| ATOM | 1475 | CG1 | VAL | B | 18 | 11.683 | 41.461 | 61.246 | 1.00 | 5.44 | B |
| ATOM | 1476 | CG2 | VAL | B | 18 | 12.267 | 40.096 | 63.273 | 1.00 | 5.67 | B |
| ATOM | 1477 | C | VAL | B | 18 | 11.522 | 42.511 | 64.938 | 1.00 | 6.04 | B |
| ATOM | 1478 | O | VAL | B | 18 | 10.339 | 42.571 | 65.264 | 1.00 | 5.94 | B |
| ATOM | 1479 | N | GLY | B | 19 | 12.513 | 42.366 | 65.817 | 1.00 | 7.93 | B |
| ATOM | 1480 | CA | GLY | B | 19 | 12.235 | 42.311 | 67.243 | 1.00 | 6.62 | B |
| ATOM | 1481 | C | GLY | B | 19 | 12.427 | 40.980 | 67.957 | 1.00 | 6.93 | B |
| ATOM | 1482 | O | GLY | B | 19 | 11.910 | 40.798 | 69.056 | 1.00 | 6.42 | B |
| ATOM | 1483 | N | LYS | B | 20 | 13.172 | 40.060 | 67.352 | 1.00 | 6.25 | B |
| ATOM | 1484 | CA | LYS | B | 20 | 13.404 | 38.746 | 67.956 | 1.00 | 6.59 | B |
| ATOM | 1485 | CB | LYS | B | 20 | 14.309 | 37.903 | 67.049 | 1.00 | 4.61 | B |
| ATOM | 1486 | CG | LYS | B | 20 | 13.738 | 37.658 | 65.654 | 1.00 | 6.26 | B |
| ATOM | 1487 | CD | LYS | B | 20 | 14.657 | 36.779 | 64.803 | 1.00 | 7.59 | B |
| ATOM | 1488 | CE | LYS | B | 20 | 16.000 | 37.453 | 64.516 | 1.00 | 9.06 | B |
| ATOM | 1489 | NZ | LYS | B | 20 | 15.854 | 38.712 | 63.716 | 1.00 | 11.35 | B |
| ATOM | 1490 | C | LYS | B | 20 | 14.011 | 38.802 | 69.359 | 1.00 | 5.71 | B |
| ATOM | 1491 | O | LYS | B | 20 | 13.538 | 38.119 | 70.276 | 1.00 | 6.11 | B |
| ATOM | 1492 | N | SER | B | 21 | 15.060 | 39.603 | 69.530 | 1.00 | 4.63 | B |
| ATOM | 1493 | CA | SER | B | 21 | 15.711 | 39.705 | 70.833 | 1.00 | 6.21 | B |
| ATOM | 1494 | CB | SER | B | 21 | 17.005 | 40.521 | 70.733 | 1.00 | 9.69 | B |
| ATOM | 1495 | OG | SER | B | 21 | 17.959 | 39.858 | 69.921 | 1.00 | 13.65 | B |
| ATOM | 1496 | C | SER | B | 21 | 14.783 | 40.338 | 71.855 | 1.00 | 5.63 | B |
| ATOM | 1497 | O | SER | B | 21 | 14.705 | 39.884 | 73.000 | 1.00 | 4.68 | B |
| ATOM | 1498 | N | SER | B | 22 | 14.081 | 41.386 | 71.434 | 1.00 | 5.12 | B |
| ATOM | 1499 | CA | SER | B | 22 | 13.147 | 42.082 | 72.304 | 1.00 | 6.23 | B |
| ATOM | 1500 | CB | SER | B | 22 | 12.521 | 43.274 | 71.572 | 1.00 | 8.07 | B |
| ATOM | 1501 | OG | SER | B | 22 | 13.493 | 44.274 | 71.302 | 1.00 | 7.39 | B |
| ATOM | 1502 | C | SER | B | 22 | 12.050 | 41.128 | 72.768 | 1.00 | 6.00 | B |
| ATOM | 1503 | O | SER | B | 22 | 11.626 | 41.173 | 73.925 | 1.00 | 4.88 | B |
| ATOM | 1504 | N | LEU | B | 23 | 11.594 | 40.263 | 71.864 | 1.00 | 4.99 | B |
| ATOM | 1505 | CA | LEU | B | 23 | 10.557 | 39.295 | 72.200 | 1.00 | 4.10 | B |
| ATOM | 1506 | CB | LEU | B | 23 | 10.093 | 38.558 | 70.940 | 1.00 | 5.00 | B |
| ATOM | 1507 | CG | LEU | B | 23 | 9.228 | 39.392 | 69.987 | 1.00 | 4.84 | B |
| ATOM | 1508 | CD1 | LEU | B | 23 | 9.039 | 38.651 | 68.667 | 1.00 | 7.62 | B |
| ATOM | 1509 | CD2 | LEU | B | 23 | 7.880 | 39.664 | 70.647 | 1.00 | 6.85 | B |
| ATOM | 1510 | C | LEU | B | 23 | 11.057 | 38.294 | 73.238 | 1.00 | 6.54 | B |
| ATOM | 1511 | O | LEU | B | 23 | 10.374 | 38.016 | 74.225 | 1.00 | 5.94 | B |
| ATOM | 1512 | N | MET | B | 24 | 12.249 | 37.752 | 73.019 | 1.00 | 6.13 | B |
| ATOM | 1513 | CA | MET | B | 24 | 12.817 | 36.793 | 73.959 | 1.00 | 6.39 | B |
| ATOM | 1514 | CB | MET | B | 24 | 14.178 | 36.291 | 73.460 | 1.00 | 7.43 | B |
| ATOM | 1515 | CG | MET | B | 24 | 14.802 | 35.260 | 74.378 | 1.00 | 12.20 | B |
| ATOM | 1516 | SD | MET | B | 24 | 16.499 | 34.845 | 73.950 | 1.00 | 15.43 | B |
| ATOM | 1517 | CE | MET | B | 24 | 16.285 | 33.481 | 72.877 | 1.00 | 19.97 | B |
| ATOM | 1518 | C | MET | B | 24 | 12.989 | 37.443 | 75.334 | 1.00 | 6.85 | B |
| ATOM | 1519 | O | MET | B | 24 | 12.648 | 36.852 | 76.360 | 1.00 | 5.34 | B |
| ATOM | 1520 | N | ASN | B | 25 | 13.523 | 38.658 | 75.356 | 1.00 | 7.17 | B |
| ATOM | 1521 | CA | ASN | B | 25 | 13.727 | 39.363 | 76.617 | 1.00 | 8.21 | B |
| ATOM | 1522 | CB | ASN | B | 25 | 14.441 | 40.694 | 76.377 | 1.00 | 9.52 | B |
| ATOM | 1523 | CG | ASN | B | 25 | 15.947 | 40.547 | 76.350 | 1.00 | 10.48 | B |
| ATOM | 1524 | OD1 | ASN | B | 25 | 16.622 | 40.758 | 77.361 | 1.00 | 11.79 | B |
| ATOM | 1525 | ND2 | ASN | B | 25 | 16.483 | 40.163 | 75.195 | 1.00 | 9.29 | B |
| ATOM | 1526 | C | ASN | B | 25 | 12.405 | 39.618 | 77.326 | 1.00 | 9.33 | B |
| ATOM | 1527 | O | ASN | B | 25 | 12.288 | 39.403 | 78.535 | 1.00 | 10.43 | B |
| ATOM | 1528 | N | ARG | B | 26 | 11.406 | 40.066 | 76.571 | 1.00 | 6.70 | B |
| ATOM | 1529 | CA | ARG | B | 26 | 10.103 | 40.350 | 77.154 | 1.00 | 8.47 | B |
| ATOM | 1530 | CB | ARG | B | 26 | 9.144 | 40.889 | 76.090 | 1.00 | 9.37 | B |
| ATOM | 1531 | CG | ARG | B | 26 | 7.809 | 41.324 | 76.657 | 1.00 | 15.14 | B |
| ATOM | 1532 | CD | ARG | B | 26 | 7.954 | 42.621 | 77.435 | 1.00 | 19.91 | B |
| ATOM | 1533 | NE | ARG | B | 26 | 6.946 | 42.753 | 78.479 | 1.00 | 26.24 | B |
| ATOM | 1534 | CZ | ARG | B | 26 | 6.584 | 43.908 | 79.026 | 1.00 | 25.49 | B |
| ATOM | 1535 | NH1 | ARG | B | 26 | 7.144 | 45.039 | 78.621 | 1.00 | 26.78 | B |
| ATOM | 1536 | NH2 | ARG | B | 26 | 5.673 | 43.929 | 79.987 | 1.00 | 28.55 | B |
| ATOM | 1537 | C | ARG | B | 26 | 9.503 | 39.104 | 77.793 | 1.00 | 9.08 | B |
| ATOM | 1538 | O | ARG | B | 26 | 8.944 | 39.165 | 78.890 | 1.00 | 10.25 | B |
| ATOM | 1539 | N | TYR | B | 27 | 9.626 | 37.970 | 77.110 | 1.00 | 8.49 | B |
| ATOM | 1540 | CA | TYR | B | 27 | 9.077 | 36.725 | 77.626 | 1.00 | 7.97 | B |
| ATOM | 1541 | CB | TYR | B | 27 | 9.105 | 35.649 | 76.539 | 1.00 | 8.01 | B |
| ATOM | 1542 | CG | TYR | B | 27 | 8.563 | 34.307 | 76.982 | 1.00 | 8.48 | B |
| ATOM | 1543 | CD1 | TYR | B | 27 | 7.302 | 34.201 | 77.570 | 1.00 | 9.35 | B |
| ATOM | 1544 | CE1 | TYR | B | 27 | 6.807 | 32.966 | 77.997 | 1.00 | 10.35 | B |
| ATOM | 1545 | CD2 | TYR | B | 27 | 9.317 | 33.143 | 76.826 | 1.00 | 8.89 | B |
| ATOM | 1546 | CE2 | TYR | B | 27 | 8.832 | 31.906 | 77.246 | 1.00 | 9.06 | B |
| ATOM | 1547 | CZ | TYR | B | 27 | 7.579 | 31.826 | 77.832 | 1.00 | 10.10 | B |
| ATOM | 1548 | OH | TYR | B | 27 | 7.103 | 30.605 | 78.261 | 1.00 | 11.44 | B |
| ATOM | 1549 | C | TYR | B | 27 | 9.810 | 36.218 | 78.867 | 1.00 | 10.10 | B |
| ATOM | 1550 | O | TYR | B | 27 | 9.183 | 35.780 | 79.835 | 1.00 | 9.66 | B |
| ATOM | 1551 | N | VAL | B | 28 | 11.135 | 36.294 | 78.845 | 1.00 | 9.42 | B |
| ATOM | 1552 | CA | VAL | B | 28 | 11.945 | 35.809 | 79.958 | 1.00 | 10.69 | B |
| ATOM | 1553 | CB | VAL | B | 28 | 13.368 | 35.447 | 79.463 | 1.00 | 10.08 | B |
| ATOM | 1554 | CG1 | VAL | B | 28 | 14.197 | 34.865 | 80.604 | 1.00 | 12.42 | B |
| ATOM | 1555 | CG2 | VAL | B | 28 | 13.275 | 34.442 | 78.323 | 1.00 | 9.71 | B |
| ATOM | 1556 | C | VAL | B | 28 | 12.063 | 36.730 | 81.180 | 1.00 | 12.43 | B |
| ATOM | 1557 | O | VAL | B | 28 | 11.927 | 36.271 | 82.315 | 1.00 | 14.82 | B |
| ATOM | 1558 | N | THR | B | 29 | 12.299 | 38.020 | 80.959 | 1.00 | 12.58 | B |
| ATOM | 1559 | CA | THR | B | 29 | 12.468 | 38.964 | 82.067 | 1.00 | 13.29 | B |
| ATOM | 1560 | CB | THR | B | 29 | 13.727 | 39.813 | 81.877 | 1.00 | 13.36 | B |
| ATOM | 1561 | OG1 | THR | B | 29 | 13.513 | 40.727 | 80.795 | 1.00 | 11.89 | B |
| ATOM | 1562 | CG2 | THR | B | 29 | 14.925 | 38.931 | 81.561 | 1.00 | 12.40 | B |
| ATOM | 1563 | C | THR | B | 29 | 11.318 | 39.946 | 82.258 | 1.00 | 14.15 | B |
| ATOM | 1564 | O | THR | B | 29 | 11.327 | 40.738 | 83.203 | 1.00 | 12.56 | B |
| ATOM | 1565 | N | ASN | B | 30 | 10.345 | 39.899 | 81.357 | 1.00 | 15.06 | B |
| ATOM | 1566 | CA | ASN | B | 30 | 9.197 | 40.797 | 81.398 | 1.00 | 16.71 | B |
| ATOM | 1567 | CB | ASN | B | 30 | 8.420 | 40.632 | 82.710 | 1.00 | 18.60 | B |
| ATOM | 1568 | CG | ASN | B | 30 | 7.016 | 41.210 | 82.628 | 1.00 | 23.48 | B |
| ATOM | 1569 | OD1 | ASN | B | 30 | 6.264 | 41.194 | 83.603 | 1.00 | 26.62 | B |
| ATOM | 1570 | ND2 | ASN | B | 30 | 6.654 | 41.718 | 81.454 | 1.00 | 23.64 | B |
| ATOM | 1571 | C | ASN | B | 30 | 9.640 | 42.251 | 81.229 | 1.00 | 15.89 | B |
| ATOM | 1572 | O | ASN | B | 30 | 8.962 | 43.178 | 81.668 | 1.00 | 16.40 | B |
| ATOM | 1573 | N | LYS | B | 31 | 10.783 | 42.438 | 80.577 | 1.00 | 15.31 | B |
| ATOM | 1574 | CA | LYS | B | 31 | 11.332 | 43.768 | 80.320 | 1.00 | 16.59 | B |
| ATOM | 1575 | CB | LYS | B | 31 | 12.736 | 43.894 | 80.915 | 1.00 | 17.29 | B |
| ATOM | 1576 | CG | LYS | B | 31 | 12.798 | 43.970 | 82.429 | 1.00 | 21.89 | B |
| ATOM | 1577 | CD | LYS | B | 31 | 14.222 | 43.705 | 82.907 | 1.00 | 23.53 | B |
| ATOM | 1578 | CE | LYS | B | 31 | 14.446 | 44.217 | 84.320 | 1.00 | 25.01 | B |
| ATOM | 1579 | NZ | LYS | B | 31 | 14.467 | 45.708 | 84.352 | 1.00 | 26.58 | B |
| ATOM | 1580 | C | LYS | B | 31 | 11.433 | 43.994 | 78.817 | 1.00 | 15.07 | B |
| ATOM | 1581 | O | LYS | B | 31 | 11.811 | 43.084 | 78.079 | 1.00 | 13.56 | B |
| ATOM | 1582 | N | PHE | B | 32 | 11.098 | 45.200 | 78.365 | 1.00 | 14.87 | B |
| ATOM | 1583 | CA | PHE | B | 32 | 11.197 | 45.517 | 76.942 | 1.00 | 13.51 | B |
| ATOM | 1584 | CB | PHE | B | 32 | 10.087 | 46.484 | 76.503 | 1.00 | 13.29 | B |
| ATOM | 1585 | CG | PHE | B | 32 | 10.253 | 47.001 | 75.094 | 1.00 | 12.57 | B |
| ATOM | 1586 | CD1 | PHE | B | 32 | 10.507 | 46.126 | 74.040 | 1.00 | 11.92 | B |
| ATOM | 1587 | CD2 | PHE | B | 32 | 10.156 | 48.362 | 74.822 | 1.00 | 12.35 | B |
| ATOM | 1588 | CE1 | PHE | B | 32 | 10.664 | 46.598 | 72.736 | 1.00 | 11.48 | B |
| ATOM | 1589 | CE2 | PHE | B | 32 | 10.311 | 48.849 | 73.517 | 1.00 | 10.64 | B |
| ATOM | 1590 | CZ | PHE | B | 32 | 10.566 | 47.964 | 72.476 | 1.00 | 12.25 | B |
| ATOM | 1591 | C | PHE | B | 32 | 12.563 | 46.128 | 76.639 | 1.00 | 14.79 | B |
| ATOM | 1592 | O | PHE | B | 32 | 12.734 | 47.350 | 76.623 | 1.00 | 14.30 | B |
| ATOM | 1593 | N | ASP | B | 33 | 13.536 | 45.257 | 76.411 | 1.00 | 14.47 | B |
| ATOM | 1594 | CA | ASP | B | 33 | 14.896 | 45.666 | 76.089 | 1.00 | 15.45 | B |
| ATOM | 1595 | CB | ASP | B | 33 | 15.595 | 46.269 | 77.318 | 1.00 | 17.65 | B |
| ATOM | 1596 | CG | ASP | B | 33 | 15.704 | 45.294 | 78.481 | 1.00 | 20.72 | B |
| ATOM | 1597 | OD1 | ASP | B | 33 | 16.135 | 44.142 | 78.266 | 1.00 | 19.45 | B |
| ATOM | 1598 | OD2 | ASP | B | 33 | 15.376 | 45.689 | 79.622 | 1.00 | 24.19 | B |
| ATOM | 1599 | C | ASP | B | 33 | 15.635 | 44.424 | 75.610 | 1.00 | 14.48 | B |
| ATOM | 1600 | O | ASP | B | 33 | 15.022 | 43.374 | 75.425 | 1.00 | 14.31 | B |
| ATOM | 1601 | N | THR | B | 34 | 16.941 | 44.537 | 75.400 | 1.00 | 14.26 | B |
| ATOM | 1602 | CA | THR | B | 34 | 17.729 | 43.392 | 74.950 | 1.00 | 14.06 | B |
| ATOM | 1603 | CB | THR | B | 34 | 18.198 | 43.578 | 73.492 | 1.00 | 16.24 | B |
| ATOM | 1604 | OG1 | THR | B | 34 | 19.015 | 44.751 | 73.397 | 1.00 | 17.67 | B |
| ATOM | 1605 | CG2 | THR | B | 34 | 16.995 | 43.727 | 72.567 | 1.00 | 12.46 | B |
| ATOM | 1606 | C | THR | B | 34 | 18.941 | 43.231 | 75.855 | 1.00 | 14.77 | B |
| ATOM | 1607 | O | THR | B | 34 | 20.055 | 42.961 | 75.394 | 1.00 | 13.60 | B |
| ATOM | 1608 | N | GLN | B | 35 | 18.698 | 43.379 | 77.153 | 1.00 | 14.04 | B |
| ATOM | 1609 | CA | GLN | B | 35 | 19.746 | 43.297 | 78.160 | 1.00 | 13.22 | B |
| ATOM | 1610 | CB | GLN | B | 35 | 19.323 | 44.097 | 79.394 | 1.00 | 14.04 | B |
| ATOM | 1611 | CG | GLN | B | 35 | 19.321 | 45.601 | 79.172 | 1.00 | 15.12 | B |
| ATOM | 1612 | CD | GLN | B | 35 | 20.715 | 46.148 | 78.928 | 1.00 | 17.16 | B |
| ATOM | 1613 | OE1 | GLN | B | 35 | 21.581 | 46.083 | 79.802 | 1.00 | 20.06 | B |
| ATOM | 1614 | NE2 | GLN | B | 35 | 20.941 | 46.685 | 77.734 | 1.00 | 18.46 | B |
| ATOM | 1615 | C | GLN | B | 35 | 20.207 | 41.907 | 78.592 | 1.00 | 12.44 | B |
| ATOM | 1616 | O | GLN | B | 35 | 21.239 | 41.792 | 79.249 | 1.00 | 10.73 | B |
| ATOM | 1617 | N | LEU | B | 36 | 19.472 | 40.853 | 78.237 | 1.00 | 11.53 | B |
| ATOM | 1618 | CA | LEU | B | 36 | 19.889 | 39.504 | 78.635 | 1.00 | 13.11 | B |
| ATOM | 1619 | CB | LEU | B | 36 | 18.924 | 38.445 | 78.094 | 1.00 | 14.15 | B |
| ATOM | 1620 | CG | LEU | B | 36 | 17.621 | 38.179 | 78.849 | 1.00 | 13.63 | B |
| ATOM | 1621 | CD1 | LEU | B | 36 | 16.765 | 37.221 | 78.031 | 1.00 | 15.69 | B |
| ATOM | 1622 | CD2 | LEU | B | 36 | 17.923 | 37.583 | 80.227 | 1.00 | 15.30 | B |
| ATOM | 1623 | C | LEU | B | 36 | 21.294 | 39.179 | 78.140 | 1.00 | 12.53 | B |
| ATOM | 1624 | O | LEU | B | 36 | 22.114 | 38.631 | 78.880 | 1.00 | 13.66 | B |
| ATOM | 1625 | N | PHE | B | 37 | 21.558 | 39.513 | 76.882 | 1.00 | 12.80 | B |
| ATOM | 1626 | CA | PHE | B | 37 | 22.853 | 39.254 | 76.265 | 1.00 | 11.53 | B |
| ATOM | 1627 | CB | PHE | B | 37 | 22.728 | 38.133 | 75.234 | 1.00 | 11.57 | B |
| ATOM | 1628 | CG | PHE | B | 37 | 22.142 | 36.864 | 75.785 | 1.00 | 10.29 | B |
| ATOM | 1629 | CD1 | PHE | B | 37 | 20.804 | 36.551 | 75.569 | 1.00 | 10.16 | B |
| ATOM | 1630 | CD2 | PHE | B | 37 | 22.928 | 35.980 | 76.519 | 1.00 | 10.18 | B |
| ATOM | 1631 | CE1 | PHE | B | 37 | 20.254 | 35.377 | 76.072 | 1.00 | 9.27 | B |
| ATOM | 1632 | CE2 | PHE | B | 37 | 22.389 | 34.799 | 77.029 | 1.00 | 8.50 | B |
| ATOM | 1633 | CZ | PHE | B | 37 | 21.043 | 34.497 | 76.802 | 1.00 | 7.93 | B |
| ATOM | 1634 | C | PHE | B | 37 | 23.348 | 40.523 | 75.586 | 1.00 | 12.23 | B |
| ATOM | 1635 | O | PHE | B | 37 | 22.585 | 41.193 | 74.887 | 1.00 | 12.52 | B |
| ATOM | 1636 | N | HIS | B | 38 | 24.627 | 40.836 | 75.775 | 1.00 | 12.75 | B |
| ATOM | 1637 | CA | HIS | B | 38 | 25.203 | 42.053 | 75.210 | 1.00 | 16.16 | B |
| ATOM | 1638 | CB | HIS | B | 38 | 26.064 | 42.744 | 76.267 | 1.00 | 17.31 | B |
| ATOM | 1639 | CG | HIS | B | 38 | 25.345 | 42.976 | 77.558 | 1.00 | 21.40 | B |
| ATOM | 1640 | CD2 | HIS | B | 38 | 25.565 | 42.487 | 78.802 | 1.00 | 23.39 | B |
| ATOM | 1641 | ND1 | HIS | B | 38 | 24.217 | 43.762 | 77.649 | 1.00 | 22.89 | B |
| ATOM | 1642 | CE1 | HIS | B | 38 | 23.770 | 43.746 | 78.892 | 1.00 | 23.84 | B |
| ATOM | 1643 | NE2 | HIS | B | 38 | 24.569 | 42.980 | 79.612 | 1.00 | 25.39 | B |
| ATOM | 1644 | C | HIS | B | 38 | 25.997 | 41.916 | 73.916 | 1.00 | 16.87 | B |
| ATOM | 1645 | O | HIS | B | 38 | 26.427 | 42.921 | 73.350 | 1.00 | 16.10 | B |
| ATOM | 1646 | N | THR | B | 39 | 26.206 | 40.692 | 73.444 | 1.00 | 17.18 | B |
| ATOM | 1647 | CA | THR | B | 39 | 26.935 | 40.510 | 72.192 | 1.00 | 17.61 | B |
| ATOM | 1648 | CB | THR | B | 39 | 28.115 | 39.520 | 72.344 | 1.00 | 18.78 | B |
| ATOM | 1649 | OG1 | THR | B | 39 | 27.623 | 38.233 | 72.731 | 1.00 | 18.46 | B |
| ATOM | 1650 | CG2 | THR | B | 39 | 29.101 | 40.024 | 73.393 | 1.00 | 17.56 | B |
| ATOM | 1651 | C | THR | B | 39 | 25.972 | 40.005 | 71.124 | 1.00 | 18.02 | B |
| ATOM | 1652 | O | THR | B | 39 | 24.951 | 39.389 | 71.440 | 1.00 | 18.77 | B |
| ATOM | 1653 | N | ILE | B | 40 | 26.293 | 40.277 | 69.864 | 1.00 | 17.37 | B |
| ATOM | 1654 | CA | ILE | B | 40 | 25.448 | 39.872 | 68.745 | 1.00 | 17.96 | B |
| ATOM | 1655 | CB | ILE | B | 40 | 25.376 | 40.999 | 67.693 | 1.00 | 19.53 | B |
| ATOM | 1656 | CG2 | ILE | B | 40 | 24.347 | 40.655 | 66.628 | 1.00 | 21.96 | B |
| ATOM | 1657 | CG1 | ILE | B | 40 | 25.011 | 42.318 | 68.379 | 1.00 | 21.02 | B |
| ATOM | 1658 | CD1 | ILE | B | 40 | 25.033 | 43.518 | 67.460 | 1.00 | 24.39 | B |
| ATOM | 1659 | C | ILE | B | 40 | 25.956 | 38.597 | 68.066 | 1.00 | 16.36 | B |
| ATOM | 1660 | O | ILE | B | 40 | 27.131 | 38.489 | 67.713 | 1.00 | 16.61 | B |
| ATOM | 1661 | N | GLY | B | 41 | 25.056 | 37.637 | 67.888 | 1.00 | 14.25 | B |
| ATOM | 1662 | CA | GLY | B | 41 | 25.411 | 36.381 | 67.253 | 1.00 | 10.38 | B |
| ATOM | 1663 | C | GLY | B | 41 | 24.270 | 35.416 | 67.479 | 1.00 | 9.00 | B |
| ATOM | 1664 | O | GLY | B | 41 | 23.200 | 35.577 | 66.896 | 1.00 | 7.25 | B |
| ATOM | 1665 | N | VAL | B | 42 | 24.500 | 34.418 | 68.329 | 1.00 | 6.72 | B |
| ATOM | 1666 | CA | VAL | B | 42 | 23.484 | 33.432 | 68.677 | 1.00 | 6.46 | B |
| ATOM | 1667 | CB | VAL | B | 42 | 23.779 | 32.040 | 68.047 | 1.00 | 5.79 | B |
| ATOM | 1668 | CG1 | VAL | B | 42 | 22.772 | 31.005 | 68.570 | 1.00 | 8.22 | B |
| ATOM | 1669 | CG2 | VAL | B | 42 | 23.696 | 32.123 | 66.526 | 1.00 | 6.69 | B |
| ATOM | 1670 | C | VAL | B | 42 | 23.491 | 33.300 | 70.198 | 1.00 | 7.42 | B |
| ATOM | 1671 | O | VAL | B | 42 | 24.549 | 33.238 | 70.823 | 1.00 | 6.33 | B |
| ATOM | 1672 | N | GLU | B | 43 | 22.310 | 33.276 | 70.801 | 1.00 | 7.99 | B |
| ATOM | 1673 | CA | GLU | B | 43 | 22.223 | 33.144 | 72.244 | 1.00 | 7.30 | B |
| ATOM | 1674 | CB | GLU | B | 43 | 22.014 | 34.517 | 72.895 | 1.00 | 10.77 | B |
| ATOM | 1675 | CG | GLU | B | 43 | 20.650 | 35.141 | 72.628 | 1.00 | 18.12 | B |
| ATOM | 1676 | CD | GLU | B | 43 | 20.738 | 36.408 | 71.795 | 1.00 | 20.47 | B |
| ATOM | 1677 | OE1 | GLU | B | 43 | 21.230 | 36.321 | 70.652 | 1.00 | 15.40 | B |
| ATOM | 1678 | OE2 | GLU | B | 43 | 20.317 | 37.487 | 72.287 | 1.00 | 24.48 | B |
| ATOM | 1679 | C | GLU | B | 43 | 21.055 | 32.236 | 72.599 | 1.00 | 5.91 | B |
| ATOM | 1680 | O | GLU | B | 43 | 20.081 | 32.168 | 71.863 | 1.00 | 5.98 | B |
| ATOM | 1681 | N | PHE | B | 44 | 21.167 | 31.523 | 73.712 | 1.00 | 4.69 | B |
| ATOM | 1682 | CA | PHE | B | 44 | 20.076 | 30.663 | 74.149 | 1.00 | 4.92 | B |
| ATOM | 1683 | CB | PHE | B | 44 | 20.122 | 29.286 | 73.461 | 1.00 | 5.31 | B |
| ATOM | 1684 | CG | PHE | B | 44 | 21.381 | 28.504 | 73.710 | 1.00 | 6.29 | B |
| ATOM | 1685 | CD1 | PHE | B | 44 | 22.522 | 28.727 | 72.943 | 1.00 | 7.27 | B |
| ATOM | 1686 | CD2 | PHE | B | 44 | 21.417 | 27.523 | 74.693 | 1.00 | 6.71 | B |
| ATOM | 1687 | CE1 | PHE | B | 44 | 23.683 | 27.976 | 73.154 | 1.00 | 6.95 | B |
| ATOM | 1688 | CE2 | PHE | B | 44 | 22.570 | 26.771 | 74.910 | 1.00 | 7.87 | B |
| ATOM | 1689 | CZ | PHE | B | 44 | 23.704 | 26.998 | 74.138 | 1.00 | 6.29 | B |
| ATOM | 1690 | C | PHE | B | 44 | 20.029 | 30.493 | 75.662 | 1.00 | 6.80 | B |
| ATOM | 1691 | O | PHE | B | 44 | 21.012 | 30.738 | 76.367 | 1.00 | 5.10 | B |
| ATOM | 1692 | N | LEU | B | 45 | 18.860 | 30.104 | 76.153 | 1.00 | 6.31 | B |
| ATOM | 1693 | CA | LEU | B | 45 | 18.658 | 29.887 | 77.580 | 1.00 | 7.29 | B |
| ATOM | 1694 | CB | LEU | B | 45 | 18.428 | 31.225 | 78.304 | 1.00 | 7.57 | B |
| ATOM | 1695 | CG | LEU | B | 45 | 17.159 | 32.051 | 78.039 | 1.00 | 10.44 | B |
| ATOM | 1696 | CD1 | LEU | B | 45 | 17.248 | 33.367 | 78.804 | 1.00 | 16.63 | B |
| ATOM | 1697 | CD2 | LEU | B | 45 | 17.009 | 32.339 | 76.565 | 1.00 | 14.97 | B |
| ATOM | 1698 | C | LEU | B | 45 | 17.466 | 28.963 | 77.790 | 1.00 | 7.50 | B |
| ATOM | 1699 | O | LEU | B | 45 | 16.705 | 28.683 | 76.853 | 1.00 | 7.55 | B |
| ATOM | 1700 | N | ASN | B | 46 | 17.320 | 28.478 | 79.018 | 1.00 | 8.02 | B |
| ATOM | 1701 | CA | ASN | B | 46 | 16.215 | 27.591 | 79.366 | 1.00 | 8.54 | B |
| ATOM | 1702 | CB | ASN | B | 46 | 16.710 | 26.392 | 80.185 | 1.00 | 8.98 | B |
| ATOM | 1703 | CG | ASN | B | 46 | 17.540 | 25.426 | 79.374 | 1.00 | 9.15 | B |
| ATOM | 1704 | OD1 | ASN | B | 46 | 17.572 | 25.494 | 78.144 | 1.00 | 9.31 | B |
| ATOM | 1705 | ND2 | ASN | B | 46 | 18.211 | 24.503 | 80.063 | 1.00 | 10.73 | B |
| ATOM | 1706 | C | ASN | B | 46 | 15.169 | 28.323 | 80.193 | 1.00 | 8.93 | B |
| ATOM | 1707 | O | ASN | B | 46 | 15.496 | 29.213 | 80.985 | 1.00 | 9.42 | B |
| ATOM | 1708 | N | LYS | B | 47 | 13.909 | 27.943 | 80.006 | 1.00 | 7.67 | B |
| ATOM | 1709 | CA | LYS | B | 47 | 12.818 | 28.516 | 80.781 | 1.00 | 8.42 | B |
| ATOM | 1710 | CB | LYS | B | 47 | 12.155 | 29.687 | 80.057 | 1.00 | 8.12 | B |
| ATOM | 1711 | CG | LYS | B | 47 | 11.013 | 30.306 | 80.873 | 1.00 | 12.48 | B |
| ATOM | 1712 | CD | LYS | B | 47 | 10.505 | 31.608 | 80.275 | 1.00 | 13.32 | B |
| ATOM | 1713 | CE | LYS | B | 47 | 9.242 | 32.083 | 80.990 | 1.00 | 13.72 | B |
| ATOM | 1714 | NZ | LYS | B | 47 | 9.453 | 32.288 | 82.454 | 1.00 | 12.61 | B |
| ATOM | 1715 | C | LYS | B | 47 | 11.792 | 27.423 | 81.026 | 1.00 | 8.87 | B |
| ATOM | 1716 | O | LYS | B | 47 | 11.328 | 26.775 | 80.089 | 1.00 | 7.56 | B |
| ATOM | 1717 | N | ASP | B | 48 | 11.452 | 27.209 | 82.291 | 1.00 | 9.81 | B |
| ATOM | 1718 | CA | ASP | B | 48 | 10.474 | 26.190 | 82.632 | 1.00 | 11.29 | B |
| ATOM | 1719 | CB | ASP | B | 48 | 10.724 | 25.636 | 84.038 | 1.00 | 12.82 | B |
| ATOM | 1720 | CG | ASP | B | 48 | 12.099 | 25.014 | 84.187 | 1.00 | 18.15 | B |
| ATOM | 1721 | OD1 | ASP | B | 48 | 12.644 | 24.513 | 83.180 | 1.00 | 17.06 | B |
| ATOM | 1722 | OD2 | ASP | B | 48 | 12.630 | 25.011 | 85.319 | 1.00 | 21.48 | B |
| ATOM | 1723 | C | ASP | B | 48 | 9.063 | 26.755 | 82.564 | 1.00 | 10.72 | B |
| ATOM | 1724 | O | ASP | B | 48 | 8.833 | 27.936 | 82.834 | 1.00 | 12.02 | B |
| ATOM | 1725 | N | LEU | B | 49 | 8.121 | 25.899 | 82.197 | 1.00 | 9.80 | B |
| ATOM | 1726 | CA | LEU | B | 49 | 6.727 | 26.290 | 82.107 | 1.00 | 11.39 | B |
| ATOM | 1727 | CB | LEU | B | 49 | 6.480 | 27.163 | 80.869 | 1.00 | 15.62 | B |
| ATOM | 1728 | CG | LEU | B | 49 | 7.030 | 26.794 | 79.488 | 1.00 | 19.43 | B |
| ATOM | 1729 | CD1 | LEU | B | 49 | 6.783 | 25.342 | 79.165 | 1.00 | 22.49 | B |
| ATOM | 1730 | CD2 | LEU | B | 49 | 6.353 | 27.683 | 78.451 | 1.00 | 21.74 | B |
| ATOM | 1731 | C | LEU | B | 49 | 5.849 | 25.054 | 82.047 | 1.00 | 10.49 | B |
| ATOM | 1732 | O | LEU | B | 49 | 6.347 | 23.930 | 81.967 | 1.00 | 10.38 | B |
| ATOM | 1733 | N | GLU | B | 50 | 4.541 | 25.268 | 82.105 | 1.00 | 10.25 | B |
| ATOM | 1734 | CA | GLU | B | 50 | 3.587 | 24.173 | 82.028 | 1.00 | 9.95 | B |
| ATOM | 1735 | CB | GLU | B | 50 | 2.809 | 24.016 | 83.335 | 1.00 | 11.15 | B |
| ATOM | 1736 | CG | GLU | B | 50 | 1.718 | 22.954 | 83.215 | 1.00 | 11.15 | B |
| ATOM | 1737 | CD | GLU | B | 50 | 0.972 | 22.686 | 84.502 | 1.00 | 13.23 | B |
| ATOM | 1738 | OE1 | GLU | B | 50 | 1.064 | 23.505 | 85.442 | 1.00 | 14.70 | B |
| ATOM | 1739 | OE2 | GLU | B | 50 | 0.278 | 21.651 | 84.560 | 1.00 | 11.45 | B |
| ATOM | 1740 | C | GLU | B | 50 | 2.620 | 24.453 | 80.891 | 1.00 | 10.98 | B |
| ATOM | 1741 | O | GLU | B | 50 | 1.948 | 25.482 | 80.878 | 1.00 | 10.34 | B |
| ATOM | 1742 | N | VAL | B | 51 | 2.566 | 23.539 | 79.929 | 1.00 | 10.84 | B |
| ATOM | 1743 | CA | VAL | B | 51 | 1.686 | 23.688 | 78.778 | 1.00 | 11.83 | B |
| ATOM | 1744 | CB | VAL | B | 51 | 2.497 | 23.890 | 77.472 | 1.00 | 12.05 | B |
| ATOM | 1745 | CG1 | VAL | B | 51 | 1.550 | 23.998 | 76.280 | 1.00 | 13.90 | B |
| ATOM | 1746 | CG2 | VAL | B | 51 | 3.366 | 25.135 | 77.582 | 1.00 | 11.99 | B |
| ATOM | 1747 | C | VAL | B | 51 | 0.833 | 22.433 | 78.640 | 1.00 | 11.95 | B |
| ATOM | 1748 | O | VAL | B | 51 | 1.354 | 21.322 | 78.613 | 1.00 | 11.03 | B |
| ATOM | 1749 | N | ASP | B | 52 | −0.479 | 22.621 | 78.563 | 1.00 | 12.47 | B |
| ATOM | 1750 | CA | ASP | B | 52 | −1.410 | 21.507 | 78.434 | 1.00 | 14.67 | B |
| ATOM | 1751 | CB | ASP | B | 52 | −1.253 | 20.846 | 77.064 | 1.00 | 18.44 | B |
| ATOM | 1752 | CG | ASP | B | 52 | −2.394 | 19.900 | 76.738 | 1.00 | 25.40 | B |
| ATOM | 1753 | OD1 | ASP | B | 52 | −3.559 | 20.361 | 76.698 | 1.00 | 28.74 | B |
| ATOM | 1754 | OD2 | ASP | B | 52 | −2.131 | 18.696 | 76.521 | 1.00 | 29.48 | B |
| ATOM | 1755 | C | ASP | B | 52 | −1.187 | 20.466 | 79.530 | 1.00 | 11.83 | B |
| ATOM | 1756 | O | ASP | B | 52 | −1.333 | 19.268 | 79.295 | 1.00 | 13.23 | B |
| ATOM | 1757 | N | GLY | B | 53 | −0.820 | 20.932 | 80.720 | 1.00 | 11.45 | B |
| ATOM | 1758 | CA | GLY | B | 53 | −0.593 | 20.031 | 81.838 | 1.00 | 10.66 | B |
| ATOM | 1759 | C | GLY | B | 53 | 0.780 | 19.392 | 81.868 | 1.00 | 11.42 | B |
| ATOM | 1760 | O | GLY | B | 53 | 1.072 | 18.571 | 82.736 | 1.00 | 12.82 | B |
| ATOM | 1761 | N | HIS | B | 54 | 1.629 | 19.771 | 80.920 | 1.00 | 11.15 | B |
| ATOM | 1762 | CA | HIS | B | 54 | 2.975 | 19.225 | 80.833 | 1.00 | 10.55 | B |
| ATOM | 1763 | CB | HIS | B | 54 | 3.342 | 18.995 | 79.364 | 1.00 | 12.51 | B |
| ATOM | 1764 | CG | HIS | B | 54 | 2.466 | 18.001 | 78.662 | 1.00 | 13.20 | B |
| ATOM | 1765 | CD2 | HIS | B | 54 | 1.372 | 18.172 | 77.882 | 1.00 | 15.61 | B |
| ATOM | 1766 | ND1 | HIS | B | 54 | 2.688 | 16.642 | 78.716 | 1.00 | 17.30 | B |
| ATOM | 1767 | CE1 | HIS | B | 54 | 1.771 | 16.018 | 77.997 | 1.00 | 18.12 | B |
| ATOM | 1768 | NE2 | HIS | B | 54 | 0.960 | 16.924 | 77.481 | 1.00 | 17.83 | B |
| ATOM | 1769 | C | HIS | B | 54 | 3.998 | 20.171 | 81.461 | 1.00 | 10.01 | B |
| ATOM | 1770 | O | HIS | B | 54 | 4.109 | 21.327 | 81.051 | 1.00 | 9.52 | B |
| ATOM | 1771 | N | PHE | B | 55 | 4.726 | 19.679 | 82.463 | 1.00 | 9.45 | B |
| ATOM | 1772 | CA | PHE | B | 55 | 5.772 | 20.457 | 83.129 | 1.00 | 9.65 | B |
| ATOM | 1773 | CB | PHE | B | 55 | 5.990 | 19.942 | 84.556 | 1.00 | 9.72 | B |
| ATOM | 1774 | CG | PHE | B | 55 | 4.991 | 20.463 | 85.552 | 1.00 | 11.47 | B |
| ATOM | 1775 | CD1 | PHE | B | 55 | 5.192 | 21.691 | 86.180 | 1.00 | 11.78 | B |
| ATOM | 1776 | CD2 | PHE | B | 55 | 3.836 | 19.745 | 85.841 | 1.00 | 10.02 | B |
| ATOM | 1777 | CE1 | PHE | B | 55 | 4.254 | 22.194 | 87.081 | 1.00 | 13.02 | B |
| ATOM | 1778 | CE2 | PHE | B | 55 | 2.892 | 20.240 | 86.740 | 1.00 | 12.14 | B |
| ATOM | 1779 | CZ | PHE | B | 55 | 3.101 | 21.468 | 87.361 | 1.00 | 11.73 | B |
| ATOM | 1780 | C | PHE | B | 55 | 7.036 | 20.243 | 82.301 | 1.00 | 9.59 | B |
| ATOM | 1781 | O | PHE | B | 55 | 7.678 | 19.197 | 82.399 | 1.00 | 8.50 | B |
| ATOM | 1782 | N | VAL | B | 56 | 7.394 | 21.231 | 81.489 | 1.00 | 9.68 | B |
| ATOM | 1783 | CA | VAL | B | 56 | 8.556 | 21.095 | 80.621 | 1.00 | 9.20 | B |
| ATOM | 1784 | CB | VAL | B | 56 | 8.105 | 20.897 | 79.158 | 1.00 | 11.50 | B |
| ATOM | 1785 | CG1 | VAL | B | 56 | 7.204 | 19.678 | 79.050 | 1.00 | 10.25 | B |
| ATOM | 1786 | CG2 | VAL | B | 56 | 7.365 | 22.128 | 78.675 | 1.00 | 11.05 | B |
| ATOM | 1787 | C | VAL | B | 56 | 9.541 | 22.259 | 80.652 | 1.00 | 9.06 | B |
| ATOM | 1788 | O | VAL | B | 56 | 9.319 | 23.277 | 81.306 | 1.00 | 7.13 | B |
| ATOM | 1789 | N | THR | B | 57 | 10.640 | 22.086 | 79.929 | 1.00 | 8.65 | B |
| ATOM | 1790 | CA | THR | B | 57 | 11.662 | 23.116 | 79.837 | 1.00 | 9.26 | B |
| ATOM | 1791 | CB | THR | B | 57 | 13.025 | 22.632 | 80.363 | 1.00 | 11.18 | B |
| ATOM | 1792 | OG1 | THR | B | 57 | 12.920 | 22.318 | 81.757 | 1.00 | 12.59 | B |
| ATOM | 1793 | CG2 | THR | B | 57 | 14.082 | 23.721 | 80.176 | 1.00 | 11.19 | B |
| ATOM | 1794 | C | THR | B | 57 | 11.821 | 23.494 | 78.375 | 1.00 | 8.87 | B |
| ATOM | 1795 | O | THR | B | 57 | 12.059 | 22.636 | 77.523 | 1.00 | 9.46 | B |
| ATOM | 1796 | N | MET | B | 58 | 11.663 | 24.780 | 78.095 | 1.00 | 9.38 | B |
| ATOM | 1797 | CA | MET | B | 58 | 11.809 | 25.299 | 76.745 | 1.00 | 9.48 | B |
| ATOM | 1798 | CB | MET | B | 58 | 10.757 | 26.380 | 76.478 | 1.00 | 12.30 | B |
| ATOM | 1799 | CG | MET | B | 58 | 10.806 | 26.977 | 75.079 | 1.00 | 16.84 | B |
| ATOM | 1800 | SD | MET | B | 58 | 9.827 | 28.499 | 74.915 | 1.00 | 19.85 | B |
| ATOM | 1801 | CE | MET | B | 58 | 8.316 | 28.032 | 75.714 | 1.00 | 20.50 | B |
| ATOM | 1802 | C | MET | B | 58 | 13.195 | 25.920 | 76.667 | 1.00 | 8.59 | B |
| ATOM | 1803 | O | MET | B | 58 | 13.633 | 26.572 | 77.610 | 1.00 | 9.84 | B |
| ATOM | 1804 | N | GLN | B | 59 | 13.895 | 25.696 | 75.561 | 1.00 | 7.73 | B |
| ATOM | 1805 | CA | GLN | B | 59 | 15.211 | 26.290 | 75.375 | 1.00 | 6.42 | B |
| ATOM | 1806 | CB | GLN | B | 59 | 16.277 | 25.221 | 75.141 | 1.00 | 6.97 | B |
| ATOM | 1807 | CG | GLN | B | 59 | 17.676 | 25.790 | 75.011 | 1.00 | 5.74 | B |
| ATOM | 1808 | CD | GLN | B | 59 | 18.730 | 24.713 | 74.983 | 1.00 | 5.39 | B |
| ATOM | 1809 | OE1 | GLN | B | 59 | 19.177 | 24.229 | 76.032 | 1.00 | 8.74 | B |
| ATOM | 1810 | NE2 | GLN | B | 59 | 19.125 | 24.311 | 73.783 | 1.00 | 1.81 | B |
| ATOM | 1811 | C | GLN | B | 59 | 15.054 | 27.171 | 74.150 | 1.00 | 6.13 | B |
| ATOM | 1812 | O | GLN | B | 59 | 14.874 | 26.685 | 73.029 | 1.00 | 6.49 | B |
| ATOM | 1813 | N | ILE | B | 60 | 15.084 | 28.475 | 74.384 | 1.00 | 6.55 | B |
| ATOM | 1814 | CA | ILE | B | 60 | 14.901 | 29.450 | 73.319 | 1.00 | 6.29 | B |
| ATOM | 1815 | CB | ILE | B | 60 | 14.191 | 30.708 | 73.856 | 1.00 | 7.37 | B |
| ATOM | 1816 | CG2 | ILE | B | 60 | 13.860 | 31.651 | 72.709 | 1.00 | 8.59 | B |
| ATOM | 1817 | CG1 | ILE | B | 60 | 12.900 | 30.313 | 74.574 | 1.00 | 7.79 | B |
| ATOM | 1818 | CD1 | ILE | B | 60 | 12.188 | 31.493 | 75.234 | 1.00 | 7.89 | B |
| ATOM | 1819 | C | ILE | B | 60 | 16.220 | 29.874 | 72.699 | 1.00 | 6.48 | B |
| ATOM | 1820 | O | ILE | B | 60 | 17.156 | 30.251 | 73.409 | 1.00 | 5.89 | B |
| ATOM | 1821 | N | TRP | B | 61 | 16.282 | 29.813 | 71.373 | 1.00 | 6.41 | B |
| ATOM | 1822 | CA | TRP | B | 61 | 17.476 | 30.207 | 70.636 | 1.00 | 6.50 | B |
| ATOM | 1823 | CB | TRP | B | 61 | 17.909 | 29.092 | 69.677 | 1.00 | 6.93 | B |
| ATOM | 1824 | CG | TRP | B | 61 | 18.555 | 27.928 | 70.365 | 1.00 | 5.57 | B |
| ATOM | 1825 | CD2 | TRP | B | 61 | 19.909 | 27.482 | 70.203 | 1.00 | 4.82 | B |
| ATOM | 1826 | CE2 | TRP | B | 61 | 20.090 | 26.371 | 71.056 | 1.00 | 5.46 | B |
| ATOM | 1827 | CE3 | TRP | B | 61 | 20.987 | 27.917 | 69.417 | 1.00 | 7.07 | B |
| ATOM | 1828 | CD1 | TRP | B | 61 | 17.984 | 27.092 | 71.284 | 1.00 | 5.37 | B |
| ATOM | 1829 | NE1 | TRP | B | 61 | 18.902 | 26.151 | 71.703 | 1.00 | 6.39 | B |
| ATOM | 1830 | CZ2 | TRP | B | 61 | 21.311 | 25.686 | 71.148 | 1.00 | 5.98 | B |
| ATOM | 1831 | CZ3 | TRP | B | 61 | 22.199 | 27.237 | 69.506 | 1.00 | 4.46 | B |
| ATOM | 1832 | CH2 | TRP | B | 61 | 22.350 | 26.132 | 70.367 | 1.00 | 5.28 | B |
| ATOM | 1833 | C | TRP | B | 61 | 17.211 | 31.483 | 69.846 | 1.00 | 8.56 | B |
| ATOM | 1834 | O | TRP | B | 61 | 16.315 | 31.533 | 69.006 | 1.00 | 9.40 | B |
| ATOM | 1835 | N | ASP | B | 62 | 17.993 | 32.516 | 70.132 | 1.00 | 6.78 | B |
| ATOM | 1836 | CA | ASP | B | 62 | 17.866 | 33.794 | 69.446 | 1.00 | 6.87 | B |
| ATOM | 1837 | CB | ASP | B | 62 | 17.980 | 34.938 | 70.462 | 1.00 | 7.48 | B |
| ATOM | 1838 | CG | ASP | B | 62 | 17.726 | 36.308 | 69.850 | 1.00 | 9.70 | B |
| ATOM | 1839 | OD1 | ASP | B | 62 | 17.118 | 36.382 | 68.764 | 1.00 | 8.94 | B |
| ATOM | 1840 | OD2 | ASP | B | 62 | 18.130 | 37.318 | 70.472 | 1.00 | 11.92 | B |
| ATOM | 1841 | C | ASP | B | 62 | 19.013 | 33.850 | 68.440 | 1.00 | 7.36 | B |
| ATOM | 1842 | O | ASP | B | 62 | 20.154 | 33.531 | 68.779 | 1.00 | 7.04 | B |
| ATOM | 1843 | N | THR | B | 63 | 18.705 | 34.243 | 67.208 | 1.00 | 7.16 | B |
| ATOM | 1844 | CA | THR | B | 63 | 19.717 | 34.334 | 66.159 | 1.00 | 8.21 | B |
| ATOM | 1845 | CB | THR | B | 63 | 19.486 | 33.265 | 65.065 | 1.00 | 9.29 | B |
| ATOM | 1846 | OG1 | THR | B | 63 | 18.230 | 33.506 | 64.412 | 1.00 | 9.90 | B |
| ATOM | 1847 | CG2 | THR | B | 63 | 19.465 | 31.869 | 65.675 | 1.00 | 10.50 | B |
| ATOM | 1848 | C | THR | B | 63 | 19.681 | 35.708 | 65.495 | 1.00 | 9.65 | B |
| ATOM | 1849 | O | THR | B | 63 | 18.626 | 36.332 | 65.413 | 1.00 | 10.71 | B |
| ATOM | 1850 | N | ALA | B | 64 | 20.838 | 36.189 | 65.049 | 1.00 | 8.91 | B |
| ATOM | 1851 | CA | ALA | B | 64 | 20.907 | 37.476 | 64.366 | 1.00 | 9.45 | B |
| ATOM | 1852 | CB | ALA | B | 64 | 22.352 | 37.961 | 64.302 | 1.00 | 10.92 | B |
| ATOM | 1853 | C | ALA | B | 64 | 20.364 | 37.198 | 62.966 | 1.00 | 9.12 | B |
| ATOM | 1854 | O | ALA | B | 64 | 20.739 | 36.206 | 62.344 | 1.00 | 10.33 | B |
| ATOM | 1855 | N | GLY | B | 65 | 19.491 | 38.072 | 62.472 | 1.00 | 8.77 | B |
| ATOM | 1856 | CA | GLY | B | 65 | 18.883 | 37.842 | 61.172 | 1.00 | 11.97 | B |
| ATOM | 1857 | C | GLY | B | 65 | 19.574 | 38.305 | 59.903 | 1.00 | 11.57 | B |
| ATOM | 1858 | O | GLY | B | 65 | 19.184 | 37.886 | 58.814 | 1.00 | 12.75 | B |
| ATOM | 1859 | N | GLN | B | 66 | 20.585 | 39.160 | 60.014 | 1.00 | 14.11 | B |
| ATOM | 1860 | CA | GLN | B | 66 | 21.278 | 39.638 | 58.819 | 1.00 | 16.19 | B |
| ATOM | 1861 | CB | GLN | B | 66 | 22.274 | 40.734 | 59.191 | 1.00 | 18.30 | B |
| ATOM | 1862 | CG | GLN | B | 66 | 21.610 | 42.054 | 59.536 | 1.00 | 24.24 | B |
| ATOM | 1863 | CD | GLN | B | 66 | 22.599 | 43.080 | 60.045 | 1.00 | 27.75 | B |
| ATOM | 1864 | OE1 | GLN | B | 66 | 23.141 | 42.943 | 61.141 | 1.00 | 31.33 | B |
| ATOM | 1865 | NE2 | GLN | B | 66 | 22.846 | 44.115 | 59.247 | 1.00 | 30.60 | B |
| ATOM | 1866 | C | GLN | B | 66 | 21.988 | 38.522 | 58.056 | 1.00 | 15.70 | B |
| ATOM | 1867 | O | GLN | B | 66 | 22.499 | 37.570 | 58.648 | 1.00 | 13.46 | B |
| ATOM | 1868 | N | GLU | B | 67 | 22.013 | 38.655 | 56.734 | 1.00 | 15.37 | B |
| ATOM | 1869 | CA | GLU | B | 67 | 22.635 | 37.672 | 55.855 | 1.00 | 16.22 | B |
| ATOM | 1870 | CB | GLU | B | 67 | 22.646 | 38.197 | 54.414 | 1.00 | 18.30 | B |
| ATOM | 1871 | CG | GLU | B | 67 | 21.263 | 38.475 | 53.847 | 0.00 | 17.57 | B |
| ATOM | 1872 | CD | GLU | B | 67 | 21.308 | 38.987 | 52.421 | 0.00 | 17.79 | B |
| ATOM | 1873 | OE1 | GLU | B | 67 | 21.822 | 38.262 | 51.544 | 0.00 | 17.74 | B |
| ATOM | 1874 | OE2 | GLU | B | 67 | 20.831 | 40.115 | 52.178 | 0.00 | 17.74 | B |
| ATOM | 1875 | C | GLU | B | 67 | 24.053 | 37.268 | 56.253 | 1.00 | 16.49 | B |
| ATOM | 1876 | O | GLU | B | 67 | 24.449 | 36.119 | 56.061 | 1.00 | 18.53 | B |
| ATOM | 1877 | N | ARG | B | 68 | 24.814 | 38.205 | 56.805 | 1.00 | 16.90 | B |
| ATOM | 1878 | CA | ARG | B | 68 | 26.188 | 37.921 | 57.196 | 1.00 | 18.59 | B |
| ATOM | 1879 | CB | ARG | B | 68 | 26.919 | 39.223 | 57.540 | 1.00 | 20.28 | B |
| ATOM | 1880 | CG | ARG | B | 68 | 26.399 | 39.917 | 58.780 | 1.00 | 25.43 | B |
| ATOM | 1881 | CD | ARG | B | 68 | 26.866 | 41.360 | 58.836 | 1.00 | 28.66 | B |
| ATOM | 1882 | NE | ARG | B | 68 | 26.310 | 42.059 | 59.990 | 1.00 | 32.42 | B |
| ATOM | 1883 | CZ | ARG | B | 68 | 26.165 | 43.379 | 60.067 | 1.00 | 34.56 | B |
| ATOM | 1884 | NH1 | ARG | B | 68 | 26.534 | 44.149 | 59.051 | 1.00 | 34.21 | B |
| ATOM | 1885 | NH2 | ARG | B | 68 | 25.657 | 43.929 | 61.163 | 1.00 | 34.03 | B |
| ATOM | 1886 | C | ARG | B | 68 | 26.297 | 36.942 | 58.365 | 1.00 | 16.41 | B |
| ATOM | 1887 | O | ARG | B | 68 | 27.383 | 36.441 | 58.653 | 1.00 | 18.03 | B |
| ATOM | 1888 | N | PHE | B | 69 | 25.185 | 36.667 | 59.039 | 1.00 | 14.00 | B |
| ATOM | 1889 | CA | PHE | B | 69 | 25.216 | 35.733 | 60.161 | 1.00 | 13.42 | B |
| ATOM | 1890 | CB | PHE | B | 69 | 24.391 | 36.261 | 61.341 | 1.00 | 13.42 | B |
| ATOM | 1891 | CG | PHE | B | 69 | 24.937 | 37.522 | 61.950 | 1.00 | 15.27 | B |
| ATOM | 1892 | CD1 | PHE | B | 69 | 24.482 | 38.767 | 61.531 | 1.00 | 15.34 | B |
| ATOM | 1893 | CD2 | PHE | B | 69 | 25.919 | 37.464 | 62.935 | 1.00 | 16.80 | B |
| ATOM | 1894 | CE1 | PHE | B | 69 | 24.997 | 39.939 | 62.085 | 1.00 | 17.58 | B |
| ATOM | 1895 | CE2 | PHE | B | 69 | 26.441 | 38.628 | 63.494 | 1.00 | 18.47 | B |
| ATOM | 1896 | CZ | PHE | B | 69 | 25.980 | 39.867 | 63.070 | 1.00 | 17.83 | B |
| ATOM | 1897 | C | PHE | B | 69 | 24.714 | 34.343 | 59.784 | 1.00 | 13.03 | B |
| ATOM | 1898 | O | PHE | B | 69 | 24.585 | 33.471 | 60.641 | 1.00 | 11.83 | B |
| ATOM | 1899 | N | ARG | B | 70 | 24.446 | 34.124 | 58.503 | 1.00 | 13.48 | B |
| ATOM | 1900 | CA | ARG | B | 70 | 23.947 | 32.823 | 58.069 | 1.00 | 14.33 | B |
| ATOM | 1901 | CB | ARG | B | 70 | 23.600 | 32.849 | 56.576 | 1.00 | 18.33 | B |
| ATOM | 1902 | CG | ARG | B | 70 | 24.767 | 33.120 | 55.651 | 1.00 | 24.14 | B |
| ATOM | 1903 | CD | ARG | B | 70 | 24.352 | 33.098 | 54.178 | 1.00 | 28.42 | B |
| ATOM | 1904 | NE | ARG | B | 70 | 23.793 | 31.811 | 53.766 | 1.00 | 31.29 | B |
| ATOM | 1905 | CZ | ARG | B | 70 | 22.519 | 31.459 | 53.915 | 1.00 | 32.10 | B |
| ATOM | 1906 | NH1 | ARG | B | 70 | 21.655 | 32.298 | 54.469 | 1.00 | 34.01 | B |
| ATOM | 1907 | NH2 | ARG | B | 70 | 22.107 | 30.265 | 53.506 | 1.00 | 32.58 | B |
| ATOM | 1908 | C | ARG | B | 70 | 24.914 | 31.675 | 58.363 | 1.00 | 13.08 | B |
| ATOM | 1909 | O | ARG | B | 70 | 24.499 | 30.632 | 58.861 | 1.00 | 12.28 | B |
| ATOM | 1910 | N | SER | B | 71 | 26.200 | 31.856 | 58.072 | 1.00 | 13.08 | B |
| ATOM | 1911 | CA | SER | B | 71 | 27.164 | 30.787 | 58.328 | 1.00 | 12.77 | B |
| ATOM | 1912 | CB | SER | B | 71 | 28.544 | 31.152 | 57.769 | 1.00 | 13.67 | B |
| ATOM | 1913 | OG | SER | B | 71 | 29.009 | 32.373 | 58.312 | 1.00 | 18.96 | B |
| ATOM | 1914 | C | SER | B | 71 | 27.273 | 30.467 | 59.817 | 1.00 | 9.94 | B |
| ATOM | 1915 | O | SER | B | 71 | 27.497 | 29.320 | 60.195 | 1.00 | 11.44 | B |
| ATOM | 1916 | N | LEU | B | 72 | 27.106 | 31.480 | 60.661 | 1.00 | 9.65 | B |
| ATOM | 1917 | CA | LEU | B | 72 | 27.179 | 31.286 | 62.104 | 1.00 | 6.52 | B |
| ATOM | 1918 | CB | LEU | B | 72 | 27.201 | 32.644 | 62.825 | 1.00 | 8.23 | B |
| ATOM | 1919 | CG | LEU | B | 72 | 26.995 | 32.629 | 64.346 | 1.00 | 8.48 | B |
| ATOM | 1920 | CD1 | LEU | B | 72 | 28.190 | 31.974 | 65.021 | 1.00 | 9.31 | B |
| ATOM | 1921 | CD2 | LEU | B | 72 | 26.806 | 34.056 | 64.864 | 1.00 | 9.20 | B |
| ATOM | 1922 | C | LEU | B | 72 | 26.011 | 30.473 | 62.658 | 1.00 | 7.45 | B |
| ATOM | 1923 | O | LEU | B | 72 | 26.210 | 29.473 | 63.348 | 1.00 | 7.62 | B |
| ATOM | 1924 | N | ARG | B | 73 | 24.795 | 30.908 | 62.336 | 1.00 | 7.83 | B |
| ATOM | 1925 | CA | ARG | B | 73 | 23.583 | 30.284 | 62.860 | 1.00 | 7.78 | B |
| ATOM | 1926 | CB | ARG | B | 73 | 22.440 | 31.314 | 62.860 | 1.00 | 6.95 | B |
| ATOM | 1927 | CG | ARG | B | 73 | 21.985 | 31.715 | 61.460 | 1.00 | 7.95 | B |
| ATOM | 1928 | CD | ARG | B | 73 | 20.800 | 32.694 | 61.436 | 1.00 | 8.84 | B |
| ATOM | 1929 | NE | ARG | B | 73 | 20.347 | 32.866 | 60.054 | 1.00 | 9.05 | B |
| ATOM | 1930 | CZ | ARG | B | 73 | 20.548 | 33.951 | 59.314 | 1.00 | 9.84 | B |
| ATOM | 1931 | NH1 | ARG | B | 73 | 21.184 | 35.001 | 59.816 | 1.00 | 9.70 | B |
| ATOM | 1932 | NH2 | ARG | B | 73 | 20.154 | 33.964 | 58.047 | 1.00 | 11.63 | B |
| ATOM | 1933 | C | ARG | B | 73 | 23.059 | 28.990 | 62.236 | 1.00 | 7.24 | B |
| ATOM | 1934 | O | ARG | B | 73 | 22.537 | 28.138 | 62.953 | 1.00 | 6.22 | B |
| ATOM | 1935 | N | THR | B | 74 | 23.192 | 28.823 | 60.922 | 1.00 | 8.33 | B |
| ATOM | 1936 | CA | THR | B | 74 | 22.629 | 27.632 | 60.291 | 1.00 | 7.36 | B |
| ATOM | 1937 | CB | THR | B | 74 | 22.817 | 27.653 | 58.746 | 1.00 | 7.30 | B |
| ATOM | 1938 | OG1 | THR | B | 74 | 24.196 | 27.822 | 58.406 | 1.00 | 9.67 | B |
| ATOM | 1939 | CG2 | THR | B | 74 | 22.006 | 28.793 | 58.142 | 1.00 | 6.65 | B |
| ATOM | 1940 | C | THR | B | 74 | 23.017 | 26.261 | 60.852 | 1.00 | 8.03 | B |
| ATOM | 1941 | O | THR | B | 74 | 22.197 | 25.345 | 60.847 | 1.00 | 8.58 | B |
| ATOM | 1942 | N | PRO | B | 75 | 24.253 | 26.092 | 61.349 | 1.00 | 8.69 | B |
| ATOM | 1943 | CD | PRO | B | 75 | 25.453 | 26.942 | 61.260 | 1.00 | 10.77 | B |
| ATOM | 1944 | CA | PRO | B | 75 | 24.582 | 24.764 | 61.885 | 1.00 | 9.04 | B |
| ATOM | 1945 | CB | PRO | B | 75 | 26.101 | 24.826 | 62.069 | 1.00 | 11.67 | B |
| ATOM | 1946 | CG | PRO | B | 75 | 26.372 | 26.288 | 62.250 | 1.00 | 15.12 | B |
| ATOM | 1947 | C | PRO | B | 75 | 23.844 | 24.438 | 63.189 | 1.00 | 8.51 | B |
| ATOM | 1948 | O | PRO | B | 75 | 23.887 | 23.306 | 63.677 | 1.00 | 9.12 | B |
| ATOM | 1949 | N | PHE | B | 76 | 23.161 | 25.428 | 63.750 | 1.00 | 5.50 | B |
| ATOM | 1950 | CA | PHE | B | 76 | 22.437 | 25.223 | 64.992 | 1.00 | 5.82 | B |
| ATOM | 1951 | CB | PHE | B | 76 | 22.776 | 26.358 | 65.963 | 1.00 | 5.21 | B |
| ATOM | 1952 | CG | PHE | B | 76 | 24.215 | 26.353 | 66.375 | 1.00 | 6.52 | B |
| ATOM | 1953 | CD1 | PHE | B | 76 | 24.665 | 25.477 | 67.357 | 1.00 | 8.03 | B |
| ATOM | 1954 | CD2 | PHE | B | 76 | 25.147 | 27.139 | 65.700 | 1.00 | 4.46 | B |
| ATOM | 1955 | CE1 | PHE | B | 76 | 26.023 | 25.375 | 67.659 | 1.00 | 8.36 | B |
| ATOM | 1956 | CE2 | PHE | B | 76 | 26.506 | 27.047 | 65.992 | 1.00 | 4.94 | B |
| ATOM | 1957 | CZ | PHE | B | 76 | 26.946 | 26.163 | 66.971 | 1.00 | 6.94 | B |
| ATOM | 1958 | C | PHE | B | 76 | 20.931 | 25.056 | 64.801 | 1.00 | 7.31 | B |
| ATOM | 1959 | O | PHE | B | 76 | 20.186 | 24.939 | 65.773 | 1.00 | 5.83 | B |
| ATOM | 1960 | N | TYR | B | 77 | 20.491 | 25.021 | 63.544 | 1.00 | 6.65 | B |
| ATOM | 1961 | CA | TYR | B | 77 | 19.073 | 24.810 | 63.246 | 1.00 | 6.86 | B |
| ATOM | 1962 | CB | TYR | B | 77 | 18.774 | 25.032 | 61.758 | 1.00 | 7.04 | B |
| ATOM | 1963 | CG | TYR | B | 77 | 18.766 | 26.468 | 61.287 | 1.00 | 5.09 | B |
| ATOM | 1964 | CD1 | TYR | B | 77 | 19.129 | 27.515 | 62.134 | 1.00 | 4.37 | B |
| ATOM | 1965 | CE1 | TYR | B | 77 | 19.143 | 28.838 | 61.675 | 1.00 | 5.78 | B |
| ATOM | 1966 | CD2 | TYR | B | 77 | 18.414 | 26.777 | 59.973 | 1.00 | 5.44 | B |
| ATOM | 1967 | CE2 | TYR | B | 77 | 18.425 | 28.091 | 59.509 | 1.00 | 5.63 | B |
| ATOM | 1968 | CZ | TYR | B | 77 | 18.791 | 29.115 | 60.363 | 1.00 | 6.44 | B |
| ATOM | 1969 | OH | TYR | B | 77 | 18.798 | 30.418 | 59.901 | 1.00 | 7.43 | B |
| ATOM | 1970 | C | TYR | B | 77 | 18.706 | 23.370 | 63.589 | 1.00 | 8.42 | B |
| ATOM | 1971 | O | TYR | B | 77 | 17.612 | 23.104 | 64.088 | 1.00 | 7.44 | B |
| ATOM | 1972 | N | ARG | B | 78 | 19.618 | 22.441 | 63.298 | 1.00 | 8.89 | B |
| ATOM | 1973 | CA | ARG | B | 78 | 19.383 | 21.026 | 63.577 | 1.00 | 11.38 | B |
| ATOM | 1974 | CB | ARG | B | 78 | 20.618 | 20.183 | 63.228 | 1.00 | 14.60 | B |
| ATOM | 1975 | CG | ARG | B | 78 | 20.702 | 19.761 | 61.770 | 1.00 | 21.44 | B |
| ATOM | 1976 | CD | ARG | B | 78 | 21.722 | 18.638 | 61.587 | 1.00 | 26.52 | B |
| ATOM | 1977 | NE | ARG | B | 78 | 21.646 | 18.036 | 60.256 | 1.00 | 30.70 | B |
| ATOM | 1978 | CZ | ARG | B | 78 | 21.929 | 18.676 | 59.125 | 1.00 | 32.41 | B |
| ATOM | 1979 | NH1 | ARG | B | 78 | 22.313 | 19.947 | 59.153 | 1.00 | 34.57 | B |
| ATOM | 1980 | NH2 | ARG | B | 78 | 21.823 | 18.046 | 57.962 | 1.00 | 33.61 | B |
| ATOM | 1981 | C | ARG | B | 78 | 19.036 | 20.812 | 65.041 | 1.00 | 9.90 | B |
| ATOM | 1982 | O | ARG | B | 78 | 19.635 | 21.424 | 65.925 | 1.00 | 9.91 | B |
| ATOM | 1983 | N | GLY | B | 79 | 18.062 | 19.944 | 65.289 | 1.00 | 10.52 | B |
| ATOM | 1984 | CA | GLY | B | 79 | 17.657 | 19.666 | 66.655 | 1.00 | 9.54 | B |
| ATOM | 1985 | C | GLY | B | 79 | 16.512 | 20.530 | 67.149 | 1.00 | 9.09 | B |
| ATOM | 1986 | O | GLY | B | 79 | 15.973 | 20.286 | 68.228 | 1.00 | 9.44 | B |
| ATOM | 1987 | N | SER | B | 80 | 16.136 | 21.543 | 66.375 | 1.00 | 8.44 | B |
| ATOM | 1988 | CA | SER | B | 80 | 15.035 | 22.415 | 66.773 | 1.00 | 7.60 | B |
| ATOM | 1989 | CB | SER | B | 80 | 14.953 | 23.640 | 65.855 | 1.00 | 8.78 | B |
| ATOM | 1990 | OG | SER | B | 80 | 16.123 | 24.435 | 65.945 | 1.00 | 8.66 | B |
| ATOM | 1991 | C | SER | B | 80 | 13.724 | 21.645 | 66.704 | 1.00 | 8.19 | B |
| ATOM | 1992 | O | SER | B | 80 | 13.514 | 20.834 | 65.796 | 1.00 | 8.08 | B |
| ATOM | 1993 | N | ASP | B | 81 | 12.845 | 21.896 | 67.667 | 1.00 | 7.00 | B |
| ATOM | 1994 | CA | ASP | B | 81 | 11.549 | 21.230 | 67.707 | 1.00 | 8.35 | B |
| ATOM | 1995 | CB | ASP | B | 81 | 11.201 | 20.869 | 69.152 | 1.00 | 7.18 | B |
| ATOM | 1996 | CG | ASP | B | 81 | 12.184 | 19.883 | 69.747 | 1.00 | 11.53 | B |
| ATOM | 1997 | OD1 | ASP | B | 81 | 12.331 | 18.783 | 69.174 | 1.00 | 12.46 | B |
| ATOM | 1998 | OD2 | ASP | B | 81 | 12.811 | 20.203 | 70.777 | 1.00 | 11.66 | B |
| ATOM | 1999 | C | ASP | B | 81 | 10.457 | 22.106 | 67.101 | 1.00 | 7.65 | B |
| ATOM | 2000 | O | ASP | B | 81 | 9.434 | 21.605 | 66.629 | 1.00 | 8.34 | B |
| ATOM | 2001 | N | CYS | B | 82 | 10.675 | 23.418 | 67.128 | 1.00 | 7.28 | B |
| ATOM | 2002 | CA | CYS | B | 82 | 9.723 | 24.371 | 66.563 | 1.00 | 6.61 | B |
| ATOM | 2003 | CB | CYS | B | 82 | 8.598 | 24.680 | 67.558 | 1.00 | 7.96 | B |
| ATOM | 2004 | SG | CYS | B | 82 | 7.365 | 25.888 | 66.965 | 1.00 | 11.25 | B |
| ATOM | 2005 | C | CYS | B | 82 | 10.451 | 25.656 | 66.213 | 1.00 | 7.37 | B |
| ATOM | 2006 | O | CYS | B | 82 | 11.442 | 26.016 | 66.850 | 1.00 | 8.72 | B |
| ATOM | 2007 | N | CYS | B | 83 | 9.961 | 26.342 | 65.191 | 1.00 | 7.34 | B |
| ATOM | 2008 | CA | CYS | B | 83 | 10.568 | 27.592 | 64.771 | 1.00 | 9.08 | B |
| ATOM | 2009 | CB | CYS | B | 83 | 11.071 | 27.481 | 63.329 | 1.00 | 11.46 | B |
| ATOM | 2010 | SG | CYS | B | 83 | 11.630 | 29.062 | 62.640 | 1.00 | 16.32 | B |
| ATOM | 2011 | C | CYS | B | 83 | 9.559 | 28.726 | 64.870 | 1.00 | 10.29 | B |
| ATOM | 2012 | O | CYS | B | 83 | 8.440 | 28.617 | 64.362 | 1.00 | 11.66 | B |
| ATOM | 2013 | N | LEU | B | 84 | 9.955 | 29.797 | 65.551 | 1.00 | 8.68 | B |
| ATOM | 2014 | CA | LEU | B | 84 | 9.113 | 30.979 | 65.702 | 1.00 | 9.35 | B |
| ATOM | 2015 | CB | LEU | B | 84 | 9.283 | 31.605 | 67.092 | 1.00 | 11.54 | B |
| ATOM | 2016 | CG | LEU | B | 84 | 8.558 | 30.982 | 68.287 | 1.00 | 15.04 | B |
| ATOM | 2017 | CD1 | LEU | B | 84 | 9.148 | 31.523 | 69.587 | 1.00 | 15.97 | B |
| ATOM | 2018 | CD2 | LEU | B | 84 | 7.072 | 31.298 | 68.206 | 1.00 | 15.12 | B |
| ATOM | 2019 | C | LEU | B | 84 | 9.580 | 31.967 | 64.646 | 1.00 | 11.08 | B |
| ATOM | 2020 | O | LEU | B | 84 | 10.519 | 32.732 | 64.881 | 1.00 | 10.94 | B |
| ATOM | 2021 | N | LEU | B | 85 | 8.946 | 31.915 | 63.476 | 1.00 | 10.22 | B |
| ATOM | 2022 | CA | LEU | B | 85 | 9.277 | 32.808 | 62.373 | 1.00 | 10.08 | B |
| ATOM | 2023 | CB | LEU | B | 85 | 8.747 | 32.271 | 61.043 | 1.00 | 14.37 | B |
| ATOM | 2024 | CG | LEU | B | 85 | 9.629 | 31.305 | 60.252 | 1.00 | 19.76 | B |
| ATOM | 2025 | CD1 | LEU | B | 85 | 8.993 | 31.052 | 58.892 | 1.00 | 19.46 | B |
| ATOM | 2026 | CD2 | LEU | B | 85 | 11.023 | 31.905 | 60.079 | 1.00 | 21.28 | B |
| ATOM | 2027 | C | LEU | B | 85 | 8.635 | 34.147 | 62.658 | 1.00 | 8.53 | B |
| ATOM | 2028 | O | LEU | B | 85 | 7.416 | 34.243 | 62.789 | 1.00 | 8.48 | B |
| ATOM | 2029 | N | THR | B | 86 | 9.463 | 35.181 | 62.721 | 1.00 | 7.46 | B |
| ATOM | 2030 | CA | THR | B | 86 | 8.990 | 36.514 | 63.047 | 1.00 | 5.23 | B |
| ATOM | 2031 | CB | THR | B | 86 | 9.712 | 37.016 | 64.322 | 1.00 | 7.83 | B |
| ATOM | 2032 | OG1 | THR | B | 86 | 9.532 | 36.060 | 65.379 | 1.00 | 11.29 | B |
| ATOM | 2033 | CG2 | THR | B | 86 | 9.169 | 38.363 | 64.762 | 1.00 | 6.22 | B |
| ATOM | 2034 | C | THR | B | 86 | 9.198 | 37.552 | 61.951 | 1.00 | 5.84 | B |
| ATOM | 2035 | O | THR | B | 86 | 10.207 | 37.542 | 61.257 | 1.00 | 4.73 | B |
| ATOM | 2036 | N | PHE | B | 87 | 8.221 | 38.434 | 61.788 | 1.00 | 4.77 | B |
| ATOM | 2037 | CA | PHE | B | 87 | 8.349 | 39.539 | 60.843 | 1.00 | 5.63 | B |
| ATOM | 2038 | CB | PHE | B | 87 | 7.596 | 39.281 | 59.521 | 1.00 | 5.10 | B |
| ATOM | 2039 | CG | PHE | B | 87 | 6.093 | 39.286 | 59.642 | 1.00 | 4.64 | B |
| ATOM | 2040 | CD1 | PHE | B | 87 | 5.412 | 38.134 | 60.019 | 1.00 | 4.58 | B |
| ATOM | 2041 | CD2 | PHE | B | 87 | 5.360 | 40.435 | 59.357 | 1.00 | 3.53 | B |
| ATOM | 2042 | CE1 | PHE | B | 87 | 4.022 | 38.116 | 60.110 | 1.00 | 5.11 | B |
| ATOM | 2043 | CE2 | PHE | B | 87 | 3.962 | 40.434 | 59.444 | 1.00 | 6.33 | B |
| ATOM | 2044 | CZ | PHE | B | 87 | 3.292 | 39.270 | 59.822 | 1.00 | 6.05 | B |
| ATOM | 2045 | C | PHE | B | 87 | 7.779 | 40.749 | 61.573 | 1.00 | 7.65 | B |
| ATOM | 2046 | O | PHE | B | 87 | 7.208 | 40.607 | 62.656 | 1.00 | 7.41 | B |
| ATOM | 2047 | N | SER | B | 88 | 7.970 | 41.939 | 61.014 | 1.00 | 7.58 | B |
| ATOM | 2048 | CA | SER | B | 88 | 7.443 | 43.150 | 61.629 | 1.00 | 7.69 | B |
| ATOM | 2049 | CB | SER | B | 88 | 8.513 | 44.241 | 61.677 | 1.00 | 8.36 | B |
| ATOM | 2050 | OG | SER | B | 88 | 7.924 | 45.492 | 62.020 | 1.00 | 9.96 | B |
| ATOM | 2051 | C | SER | B | 88 | 6.275 | 43.627 | 60.784 | 1.00 | 7.48 | B |
| ATOM | 2052 | O | SER | B | 88 | 6.404 | 43.750 | 59.566 | 1.00 | 6.35 | B |
| ATOM | 2053 | N | VAL | B | 89 | 5.135 | 43.901 | 61.414 | 1.00 | 7.01 | B |
| ATOM | 2054 | CA | VAL | B | 89 | 3.981 | 44.357 | 60.647 | 1.00 | 9.57 | B |
| ATOM | 2055 | CB | VAL | B | 89 | 2.690 | 44.387 | 61.504 | 1.00 | 8.09 | B |
| ATOM | 2056 | CG1 | VAL | B | 89 | 2.356 | 42.979 | 61.983 | 1.00 | 6.92 | B |
| ATOM | 2057 | CG2 | VAL | B | 89 | 2.849 | 45.340 | 62.678 | 1.00 | 7.28 | B |
| ATOM | 2058 | C | VAL | B | 89 | 4.209 | 45.730 | 60.018 | 1.00 | 10.67 | B |
| ATOM | 2059 | O | VAL | B | 89 | 3.424 | 46.169 | 59.180 | 1.00 | 10.26 | B |
| ATOM | 2060 | N | ASP | B | 90 | 5.281 | 46.413 | 60.406 | 1.00 | 12.31 | B |
| ATOM | 2061 | CA | ASP | B | 90 | 5.543 | 47.720 | 59.812 | 1.00 | 15.45 | B |
| ATOM | 2062 | CB | ASP | B | 90 | 5.946 | 48.742 | 60.887 | 1.00 | 19.71 | B |
| ATOM | 2063 | CG | ASP | B | 90 | 7.370 | 48.567 | 61.368 | 1.00 | 25.60 | B |
| ATOM | 2064 | OD1 | ASP | B | 90 | 7.832 | 49.410 | 62.172 | 1.00 | 30.21 | B |
| ATOM | 2065 | OD2 | ASP | B | 90 | 8.032 | 47.596 | 60.948 | 1.00 | 29.42 | B |
| ATOM | 2066 | C | ASP | B | 90 | 6.619 | 47.639 | 58.729 | 1.00 | 15.27 | B |
| ATOM | 2067 | O | ASP | B | 90 | 7.044 | 48.664 | 58.187 | 1.00 | 14.75 | B |
| ATOM | 2068 | N | ASP | B | 91 | 7.045 | 46.419 | 58.409 | 1.00 | 13.49 | B |
| ATOM | 2069 | CA | ASP | B | 91 | 8.077 | 46.204 | 57.398 | 1.00 | 14.61 | B |
| ATOM | 2070 | CB | ASP | B | 91 | 9.408 | 45.855 | 58.084 | 1.00 | 16.36 | B |
| ATOM | 2071 | CG | ASP | B | 91 | 10.515 | 45.514 | 57.094 | 1.00 | 19.56 | B |
| ATOM | 2072 | OD1 | ASP | B | 91 | 10.379 | 45.836 | 55.895 | 1.00 | 23.49 | B |
| ATOM | 2073 | OD2 | ASP | B | 91 | 11.534 | 44.931 | 57.525 | 1.00 | 22.67 | B |
| ATOM | 2074 | C | ASP | B | 91 | 7.672 | 45.101 | 56.427 | 1.00 | 12.97 | B |
| ATOM | 2075 | O | ASP | B | 91 | 7.918 | 43.921 | 56.675 | 1.00 | 12.53 | B |
| ATOM | 2076 | N | SER | B | 92 | 7.054 | 45.497 | 55.317 | 1.00 | 10.58 | B |
| ATOM | 2077 | CA | SER | B | 92 | 6.597 | 44.548 | 54.306 | 1.00 | 11.95 | B |
| ATOM | 2078 | CB | SER | B | 92 | 6.001 | 45.299 | 53.111 | 1.00 | 14.96 | B |
| ATOM | 2079 | OG | SER | B | 92 | 4.932 | 46.130 | 53.530 | 1.00 | 20.34 | B |
| ATOM | 2080 | C | SER | B | 92 | 7.700 | 43.615 | 53.818 | 1.00 | 11.69 | B |
| ATOM | 2081 | O | SER | B | 92 | 7.446 | 42.453 | 53.524 | 1.00 | 12.34 | B |
| ATOM | 2082 | N | GLN | B | 93 | 8.921 | 44.128 | 53.725 | 1.00 | 11.71 | B |
| ATOM | 2083 | CA | GLN | B | 93 | 10.042 | 43.318 | 53.269 | 1.00 | 12.68 | B |
| ATOM | 2084 | CB | GLN | B | 93 | 11.327 | 44.144 | 53.283 | 1.00 | 16.34 | B |
| ATOM | 2085 | CG | GLN | B | 93 | 12.476 | 43.515 | 52.520 | 1.00 | 20.21 | B |
| ATOM | 2086 | CD | GLN | B | 93 | 12.851 | 44.327 | 51.298 | 1.00 | 24.41 | B |
| ATOM | 2087 | OE1 | GLN | B | 93 | 13.316 | 45.465 | 51.413 | 1.00 | 25.75 | B |
| ATOM | 2088 | NE2 | GLN | B | 93 | 12.640 | 43.753 | 50.118 | 1.00 | 25.02 | B |
| ATOM | 2089 | C | GLN | B | 93 | 10.221 | 42.100 | 54.166 | 1.00 | 11.27 | B |
| ATOM | 2090 | O | GLN | B | 93 | 10.430 | 40.988 | 53.680 | 1.00 | 9.78 | B |
| ATOM | 2091 | N | SER | B | 94 | 10.135 | 42.312 | 55.478 | 1.00 | 8.48 | B |
| ATOM | 2092 | CA | SER | B | 94 | 10.309 | 41.218 | 56.431 | 1.00 | 7.86 | B |
| ATOM | 2093 | CB | SER | B | 94 | 10.320 | 41.743 | 57.877 | 1.00 | 4.11 | B |
| ATOM | 2094 | OG | SER | B | 94 | 9.064 | 42.270 | 58.279 | 1.00 | 4.54 | B |
| ATOM | 2095 | C | SER | B | 94 | 9.242 | 40.143 | 56.271 | 1.00 | 7.00 | B |
| ATOM | 2096 | O | SER | B | 94 | 9.502 | 38.966 | 56.520 | 1.00 | 8.81 | B |
| ATOM | 2097 | N | PHE | B | 95 | 8.048 | 40.552 | 55.850 | 1.00 | 7.57 | B |
| ATOM | 2098 | CA | PHE | B | 95 | 6.942 | 39.616 | 55.643 | 1.00 | 8.41 | B |
| ATOM | 2099 | CB | PHE | B | 95 | 5.616 | 40.384 | 55.567 | 1.00 | 6.48 | B |
| ATOM | 2100 | CG | PHE | B | 95 | 4.436 | 39.526 | 55.208 | 1.00 | 7.40 | B |
| ATOM | 2101 | CD1 | PHE | B | 95 | 3.987 | 38.539 | 56.078 | 1.00 | 6.80 | B |
| ATOM | 2102 | CD2 | PHE | B | 95 | 3.787 | 39.693 | 53.988 | 1.00 | 7.20 | B |
| ATOM | 2103 | CE1 | PHE | B | 95 | 2.905 | 37.725 | 55.741 | 1.00 | 6.15 | B |
| ATOM | 2104 | CE2 | PHE | B | 95 | 2.706 | 38.888 | 53.640 | 1.00 | 7.51 | B |
| ATOM | 2105 | CZ | PHE | B | 95 | 2.265 | 37.901 | 54.518 | 1.00 | 7.23 | B |
| ATOM | 2106 | C | PHE | B | 95 | 7.164 | 38.838 | 54.345 | 1.00 | 8.62 | B |
| ATOM | 2107 | O | PHE | B | 95 | 6.975 | 37.624 | 54.293 | 1.00 | 8.15 | B |
| ATOM | 2108 | N | GLN | B | 96 | 7.564 | 39.547 | 53.297 | 1.00 | 9.39 | B |
| ATOM | 2109 | CA | GLN | B | 96 | 7.812 | 38.924 | 51.994 | 1.00 | 10.21 | B |
| ATOM | 2110 | CB | GLN | B | 96 | 8.211 | 39.996 | 50.977 | 1.00 | 11.63 | B |
| ATOM | 2111 | CG | GLN | B | 96 | 7.120 | 41.021 | 50.701 | 1.00 | 17.92 | B |
| ATOM | 2112 | CD | GLN | B | 96 | 7.650 | 42.243 | 49.975 | 1.00 | 21.20 | B |
| ATOM | 2113 | OE1 | GLN | B | 96 | 8.348 | 42.124 | 48.968 | 1.00 | 25.73 | B |
| ATOM | 2114 | NE2 | GLN | B | 96 | 7.316 | 43.425 | 50.480 | 1.00 | 24.42 | B |
| ATOM | 2115 | C | GLN | B | 96 | 8.915 | 37.873 | 52.073 | 1.00 | 9.97 | B |
| ATOM | 2116 | O | GLN | B | 96 | 8.963 | 36.942 | 51.267 | 1.00 | 10.20 | B |
| ATOM | 2117 | N | ASN | B | 97 | 9.797 | 38.028 | 53.052 | 1.00 | 7.64 | B |
| ATOM | 2118 | CA | ASN | B | 97 | 10.907 | 37.106 | 53.232 | 1.00 | 8.76 | B |
| ATOM | 2119 | CB | ASN | B | 97 | 12.073 | 37.835 | 53.908 | 1.00 | 10.35 | B |
| ATOM | 2120 | CG | ASN | B | 97 | 12.848 | 38.719 | 52.943 | 1.00 | 13.60 | B |
| ATOM | 2121 | OD1 | ASN | B | 97 | 13.515 | 39.674 | 53.352 | 1.00 | 15.48 | B |
| ATOM | 2122 | ND2 | ASN | B | 97 | 12.775 | 38.395 | 51.657 | 1.00 | 14.14 | B |
| ATOM | 2123 | C | ASN | B | 97 | 10.561 | 35.846 | 54.026 | 1.00 | 7.33 | B |
| ATOM | 2124 | O | ASN | B | 97 | 11.411 | 34.981 | 54.193 | 1.00 | 8.94 | B |
| ATOM | 2125 | N | LEU | B | 98 | 9.325 | 35.728 | 54.505 | 1.00 | 8.54 | B |
| ATOM | 2126 | CA | LEU | B | 98 | 8.948 | 34.551 | 55.287 | 1.00 | 8.61 | B |
| ATOM | 2127 | CB | LEU | B | 98 | 7.475 | 34.615 | 55.702 | 1.00 | 7.58 | B |
| ATOM | 2128 | CG | LEU | B | 98 | 7.123 | 35.653 | 56.769 | 1.00 | 10.85 | B |
| ATOM | 2129 | CD1 | LEU | B | 98 | 5.634 | 35.571 | 57.091 | 1.00 | 11.38 | B |
| ATOM | 2130 | CD2 | LEU | B | 98 | 7.954 | 35.407 | 58.017 | 1.00 | 11.78 | B |
| ATOM | 2131 | C | LEU | B | 98 | 9.205 | 33.243 | 54.554 | 1.00 | 9.08 | B |
| ATOM | 2132 | O | LEU | B | 98 | 9.746 | 32.299 | 55.133 | 1.00 | 10.52 | B |
| ATOM | 2133 | N | SER | B | 99 | 8.811 | 33.179 | 53.286 | 1.00 | 9.39 | B |
| ATOM | 2134 | CA | SER | B | 99 | 9.010 | 31.966 | 52.503 | 1.00 | 10.54 | B |
| ATOM | 2135 | CB | SER | B | 99 | 8.490 | 32.172 | 51.078 | 1.00 | 14.72 | B |
| ATOM | 2136 | OG | SER | B | 99 | 9.095 | 33.306 | 50.480 | 1.00 | 20.15 | B |
| ATOM | 2137 | C | SER | B | 99 | 10.495 | 31.595 | 52.483 | 1.00 | 10.15 | B |
| ATOM | 2138 | O | SER | B | 99 | 10.855 | 30.430 | 52.655 | 1.00 | 9.27 | B |
| ATOM | 2139 | N | ASN | B | 100 | 11.356 | 32.592 | 52.292 | 1.00 | 9.62 | B |
| ATOM | 2140 | CA | ASN | B | 100 | 12.794 | 32.348 | 52.262 | 1.00 | 11.17 | B |
| ATOM | 2141 | CB | ASN | B | 100 | 13.553 | 33.623 | 51.894 | 1.00 | 14.50 | B |
| ATOM | 2142 | CG | ASN | B | 100 | 13.332 | 34.033 | 50.453 | 1.00 | 18.87 | B |
| ATOM | 2143 | OD1 | ASN | B | 100 | 13.272 | 33.187 | 49.560 | 1.00 | 20.45 | B |
| ATOM | 2144 | ND2 | ASN | B | 100 | 13.222 | 35.335 | 50.216 | 1.00 | 21.75 | B |
| ATOM | 2145 | C | ASN | B | 100 | 13.311 | 31.813 | 53.591 | 1.00 | 9.93 | B |
| ATOM | 2146 | O | ASN | B | 100 | 14.185 | 30.947 | 53.619 | 1.00 | 6.64 | B |
| ATOM | 2147 | N | TRP | B | 101 | 12.781 | 32.329 | 54.694 | 1.00 | 8.83 | B |
| ATOM | 2148 | CA | TRP | B | 101 | 13.213 | 31.851 | 56.000 | 1.00 | 7.82 | B |
| ATOM | 2149 | CB | TRP | B | 101 | 12.642 | 32.723 | 57.125 | 1.00 | 6.58 | B |
| ATOM | 2150 | CG | TRP | B | 101 | 13.434 | 33.972 | 57.352 | 1.00 | 7.18 | B |
| ATOM | 2151 | CD2 | TRP | B | 101 | 14.742 | 34.063 | 57.934 | 1.00 | 7.46 | B |
| ATOM | 2152 | CE2 | TRP | B | 101 | 15.100 | 35.428 | 57.941 | 1.00 | 5.97 | B |
| ATOM | 2153 | CE3 | TRP | B | 101 | 15.646 | 33.123 | 58.449 | 1.00 | 6.55 | B |
| ATOM | 2154 | CD1 | TRP | B | 101 | 13.065 | 35.246 | 57.037 | 1.00 | 6.45 | B |
| ATOM | 2155 | NE1 | TRP | B | 101 | 14.059 | 36.127 | 57.388 | 1.00 | 8.69 | B |
| ATOM | 2156 | CZ2 | TRP | B | 101 | 16.327 | 35.881 | 58.446 | 1.00 | 7.67 | B |
| ATOM | 2157 | CZ3 | TRP | B | 101 | 16.869 | 33.574 | 58.952 | 1.00 | 8.34 | B |
| ATOM | 2158 | CH2 | TRP | B | 101 | 17.195 | 34.943 | 58.946 | 1.00 | 7.76 | B |
| ATOM | 2159 | C | TRP | B | 101 | 12.790 | 30.405 | 56.198 | 1.00 | 6.74 | B |
| ATOM | 2160 | O | TRP | B | 101 | 13.563 | 29.589 | 56.698 | 1.00 | 7.01 | B |
| ATOM | 2161 | N | LYS | B | 102 | 11.563 | 30.079 | 55.803 | 1.00 | 7.72 | B |
| ATOM | 2162 | CA | LYS | B | 102 | 11.087 | 28.709 | 55.947 | 1.00 | 8.58 | B |
| ATOM | 2163 | CB | LYS | B | 102 | 9.656 | 28.564 | 55.421 | 1.00 | 8.21 | B |
| ATOM | 2164 | CG | LYS | B | 102 | 9.049 | 27.199 | 55.741 | 1.00 | 12.52 | B |
| ATOM | 2165 | CD | LYS | B | 102 | 7.828 | 26.849 | 54.896 | 1.00 | 17.34 | B |
| ATOM | 2166 | CE | LYS | B | 102 | 6.868 | 28.014 | 54.729 | 1.00 | 20.31 | B |
| ATOM | 2167 | NZ | LYS | B | 102 | 7.369 | 29.020 | 53.744 | 1.00 | 25.01 | B |
| ATOM | 2168 | C | LYS | B | 102 | 11.995 | 27.761 | 55.165 | 1.00 | 8.86 | B |
| ATOM | 2169 | O | LYS | B | 102 | 12.429 | 26.731 | 55.683 | 1.00 | 6.91 | B |
| ATOM | 2170 | N | LYS | B | 103 | 12.280 | 28.108 | 53.913 | 1.00 | 7.24 | B |
| ATOM | 2171 | CA | LYS | B | 103 | 13.136 | 27.264 | 53.084 | 1.00 | 8.19 | B |
| ATOM | 2172 | CB | LYS | B | 103 | 13.235 | 27.824 | 51.661 | 1.00 | 11.67 | B |
| ATOM | 2173 | CG | LYS | B | 103 | 11.970 | 27.620 | 50.842 | 1.00 | 17.03 | B |
| ATOM | 2174 | CD | LYS | B | 103 | 12.168 | 28.034 | 49.391 | 1.00 | 21.54 | B |
| ATOM | 2175 | CE | LYS | B | 103 | 10.933 | 27.727 | 48.557 | 1.00 | 24.03 | B |
| ATOM | 2176 | NZ | LYS | B | 103 | 11.144 | 28.072 | 47.121 | 1.00 | 25.25 | B |
| ATOM | 2177 | C | LYS | B | 103 | 14.531 | 27.087 | 53.667 | 1.00 | 6.81 | B |
| ATOM | 2178 | O | LYS | B | 103 | 15.093 | 25.985 | 53.624 | 1.00 | 7.73 | B |
| ATOM | 2179 | N | GLU | B | 104 | 15.090 | 28.161 | 54.217 | 1.00 | 5.17 | B |
| ATOM | 2180 | CA | GLU | B | 104 | 16.424 | 28.100 | 54.803 | 1.00 | 6.00 | B |
| ATOM | 2181 | CB | GLU | B | 104 | 16.903 | 29.500 | 55.205 | 1.00 | 7.56 | B |
| ATOM | 2182 | CG | GLU | B | 104 | 18.315 | 29.525 | 55.790 | 1.00 | 9.99 | B |
| ATOM | 2183 | CD | GLU | B | 104 | 18.748 | 30.920 | 56.210 | 1.00 | 11.57 | B |
| ATOM | 2184 | OE1 | GLU | B | 104 | 18.768 | 31.824 | 55.346 | 1.00 | 14.95 | B |
| ATOM | 2185 | OE2 | GLU | B | 104 | 19.066 | 31.116 | 57.402 | 1.00 | 11.18 | B |
| ATOM | 2186 | C | GLU | B | 104 | 16.420 | 27.189 | 56.027 | 1.00 | 6.61 | B |
| ATOM | 2187 | O | GLU | B | 104 | 17.325 | 26.373 | 56.209 | 1.00 | 7.28 | B |
| ATOM | 2188 | N | PHE | B | 105 | 15.396 | 27.313 | 56.865 | 1.00 | 7.59 | B |
| ATOM | 2189 | CA | PHE | B | 105 | 15.338 | 26.472 | 58.049 | 1.00 | 7.18 | B |
| ATOM | 2190 | CB | PHE | B | 105 | 14.135 | 26.814 | 58.928 | 1.00 | 7.61 | B |
| ATOM | 2191 | CG | PHE | B | 105 | 13.985 | 25.887 | 60.098 | 1.00 | 9.75 | B |
| ATOM | 2192 | CD1 | PHE | B | 105 | 14.823 | 26.005 | 61.205 | 1.00 | 9.93 | B |
| ATOM | 2193 | CD2 | PHE | B | 105 | 13.065 | 24.841 | 60.061 | 1.00 | 10.60 | B |
| ATOM | 2194 | CE1 | PHE | B | 105 | 14.752 | 25.093 | 62.258 | 1.00 | 10.49 | B |
| ATOM | 2195 | CE2 | PHE | B | 105 | 12.984 | 23.918 | 61.110 | 1.00 | 12.88 | B |
| ATOM | 2196 | CZ | PHE | B | 105 | 13.830 | 24.044 | 62.210 | 1.00 | 12.38 | B |
| ATOM | 2197 | C | PHE | B | 105 | 15.266 | 24.994 | 57.685 | 1.00 | 7.81 | B |
| ATOM | 2198 | O | PHE | B | 105 | 16.075 | 24.193 | 58.155 | 1.00 | 8.77 | B |
| ATOM | 2199 | N | ILE | B | 106 | 14.300 | 24.639 | 56.842 | 1.00 | 8.48 | B |
| ATOM | 2200 | CA | ILE | B | 106 | 14.121 | 23.252 | 56.444 | 1.00 | 9.50 | B |
| ATOM | 2201 | CB | ILE | B | 106 | 12.944 | 23.091 | 55.450 | 1.00 | 10.84 | B |
| ATOM | 2202 | CG2 | ILE | B | 106 | 11.627 | 23.429 | 56.145 | 1.00 | 11.31 | B |
| ATOM | 2203 | CG1 | ILE | B | 106 | 13.165 | 23.974 | 54.224 | 1.00 | 16.12 | B |
| ATOM | 2204 | CD1 | ILE | B | 106 | 12.086 | 23.824 | 53.157 | 1.00 | 20.06 | B |
| ATOM | 2205 | C | ILE | B | 106 | 15.380 | 22.646 | 55.837 | 1.00 | 8.94 | B |
| ATOM | 2206 | O | ILE | B | 106 | 15.735 | 21.510 | 56.155 | 1.00 | 9.65 | B |
| ATOM | 2207 | N | TYR | B | 107 | 16.063 | 23.400 | 54.981 | 1.00 | 8.48 | B |
| ATOM | 2208 | CA | TYR | B | 107 | 17.287 | 22.905 | 54.347 | 1.00 | 9.35 | B |
| ATOM | 2209 | CB | TYR | B | 107 | 17.856 | 23.947 | 53.375 | 1.00 | 9.30 | B |
| ATOM | 2210 | CG | TYR | B | 107 | 19.081 | 23.464 | 52.627 | 1.00 | 10.31 | B |
| ATOM | 2211 | CD1 | TYR | B | 107 | 18.959 | 22.579 | 51.556 | 1.00 | 11.18 | B |
| ATOM | 2212 | CE1 | TYR | B | 107 | 20.084 | 22.100 | 50.882 | 1.00 | 12.16 | B |
| ATOM | 2213 | CD2 | TYR | B | 107 | 20.361 | 23.861 | 53.009 | 1.00 | 10.94 | B |
| ATOM | 2214 | CE2 | TYR | B | 107 | 21.497 | 23.386 | 52.343 | 1.00 | 9.79 | B |
| ATOM | 2215 | CZ | TYR | B | 107 | 21.348 | 22.504 | 51.280 | 1.00 | 13.58 | B |
| ATOM | 2216 | OH | TYR | B | 107 | 22.456 | 22.016 | 50.617 | 1.00 | 12.30 | B |
| ATOM | 2217 | C | TYR | B | 107 | 18.374 | 22.541 | 55.361 | 1.00 | 9.85 | B |
| ATOM | 2218 | O | TYR | B | 107 | 18.886 | 21.420 | 55.362 | 1.00 | 9.80 | B |
| ATOM | 2219 | N | TYR | B | 108 | 18.726 | 23.492 | 56.221 | 1.00 | 9.50 | B |
| ATOM | 2220 | CA | TYR | B | 108 | 19.782 | 23.274 | 57.211 | 1.00 | 10.55 | B |
| ATOM | 2221 | CB | TYR | B | 108 | 20.339 | 24.620 | 57.677 | 1.00 | 10.16 | B |
| ATOM | 2222 | CG | TYR | B | 108 | 21.124 | 25.342 | 56.609 | 1.00 | 9.90 | B |
| ATOM | 2223 | CD1 | TYR | B | 108 | 22.413 | 24.934 | 56.263 | 1.00 | 9.34 | B |
| ATOM | 2224 | CE1 | TYR | B | 108 | 23.126 | 25.588 | 55.262 | 1.00 | 9.15 | B |
| ATOM | 2225 | CD2 | TYR | B | 108 | 20.571 | 26.421 | 55.925 | 1.00 | 9.44 | B |
| ATOM | 2226 | CE2 | TYR | B | 108 | 21.274 | 27.077 | 54.925 | 1.00 | 10.47 | B |
| ATOM | 2227 | CZ | TYR | B | 108 | 22.548 | 26.657 | 54.597 | 1.00 | 11.11 | B |
| ATOM | 2228 | OH | TYR | B | 108 | 23.236 | 27.302 | 53.595 | 1.00 | 11.75 | B |
| ATOM | 2229 | C | TYR | B | 108 | 19.399 | 22.438 | 58.420 | 1.00 | 11.16 | B |
| ATOM | 2230 | O | TYR | B | 108 | 20.259 | 21.807 | 59.036 | 1.00 | 10.34 | B |
| ATOM | 2231 | N | ALA | B | 109 | 18.118 | 22.427 | 58.771 | 1.00 | 11.58 | B |
| ATOM | 2232 | CA | ALA | B | 109 | 17.680 | 21.636 | 59.916 | 1.00 | 15.10 | B |
| ATOM | 2233 | CB | ALA | B | 109 | 16.374 | 22.189 | 60.472 | 1.00 | 15.12 | B |
| ATOM | 2234 | C | ALA | B | 109 | 17.496 | 20.182 | 59.496 | 1.00 | 17.82 | B |
| ATOM | 2235 | O | ALA | B | 109 | 17.444 | 19.290 | 60.338 | 1.00 | 19.18 | B |
| ATOM | 2236 | N | ASP | B | 110 | 17.407 | 19.964 | 58.187 | 1.00 | 20.88 | B |
| ATOM | 2237 | CA | ASP | B | 110 | 17.215 | 18.638 | 57.607 | 1.00 | 24.47 | B |
| ATOM | 2238 | CB | ASP | B | 110 | 18.340 | 17.691 | 58.037 | 1.00 | 26.43 | B |
| ATOM | 2239 | CG | ASP | B | 110 | 18.292 | 16.361 | 57.303 | 1.00 | 29.83 | B |
| ATOM | 2240 | OD1 | ASP | B | 110 | 18.360 | 16.367 | 56.054 | 1.00 | 30.52 | B |
| ATOM | 2241 | OD2 | ASP | B | 110 | 18.185 | 15.311 | 57.974 | 1.00 | 31.55 | B |
| ATOM | 2242 | C | ASP | B | 110 | 15.862 | 18.056 | 58.013 | 1.00 | 26.13 | B |
| ATOM | 2243 | O | ASP | B | 110 | 14.812 | 18.640 | 57.719 | 1.00 | 27.08 | B |
| ATOM | 2244 | N | GLU | B | 115 | 8.295 | 19.442 | 55.359 | 1.00 | 33.03 | B |
| ATOM | 2245 | CA | GLU | B | 115 | 7.322 | 18.429 | 55.749 | 1.00 | 32.28 | B |
| ATOM | 2246 | CB | GLU | B | 115 | 8.002 | 17.350 | 56.590 | 1.00 | 32.52 | B |
| ATOM | 2247 | CG | GLU | B | 115 | 8.879 | 17.902 | 57.697 | 1.00 | 34.45 | B |
| ATOM | 2248 | CD | GLU | B | 115 | 9.102 | 16.903 | 58.813 | 1.00 | 35.29 | B |
| ATOM | 2249 | OE1 | GLU | B | 115 | 8.137 | 16.624 | 59.555 | 1.00 | 34.52 | B |
| ATOM | 2250 | OE2 | GLU | B | 115 | 10.237 | 16.394 | 58.945 | 1.00 | 36.20 | B |
| ATOM | 2251 | C | GLU | B | 115 | 6.163 | 19.038 | 56.536 | 1.00 | 31.43 | B |
| ATOM | 2252 | O | GLU | B | 115 | 5.496 | 19.959 | 56.061 | 1.00 | 32.96 | B |
| ATOM | 2253 | N | SER | B | 116 | 5.926 | 18.511 | 57.736 | 1.00 | 28.89 | B |
| ATOM | 2254 | CA | SER | B | 116 | 4.860 | 18.994 | 58.610 | 1.00 | 26.00 | B |
| ATOM | 2255 | CB | SER | B | 116 | 3.909 | 17.853 | 58.967 | 1.00 | 27.63 | B |
| ATOM | 2256 | OG | SER | B | 116 | 4.574 | 16.875 | 59.748 | 1.00 | 29.97 | B |
| ATOM | 2257 | C | SER | B | 116 | 5.476 | 19.551 | 59.890 | 1.00 | 23.25 | B |
| ATOM | 2258 | O | SER | B | 116 | 4.886 | 19.469 | 60.968 | 1.00 | 23.09 | B |
| ATOM | 2259 | N | PHE | B | 117 | 6.674 | 20.106 | 59.764 | 1.00 | 19.91 | B |
| ATOM | 2260 | CA | PHE | B | 117 | 7.376 | 20.675 | 60.905 | 1.00 | 17.92 | B |
| ATOM | 2261 | CB | PHE | B | 117 | 8.751 | 21.179 | 60.480 | 1.00 | 15.78 | B |
| ATOM | 2262 | CG | PHE | B | 117 | 9.616 | 21.578 | 61.627 | 1.00 | 14.00 | B |
| ATOM | 2263 | CD1 | PHE | B | 117 | 10.368 | 20.625 | 62.309 | 1.00 | 13.81 | B |
| ATOM | 2264 | CD2 | PHE | B | 117 | 9.649 | 22.897 | 62.060 | 1.00 | 14.38 | B |
| ATOM | 2265 | CE1 | PHE | B | 117 | 11.140 | 20.982 | 63.408 | 1.00 | 15.34 | B |
| ATOM | 2266 | CE2 | PHE | B | 117 | 10.416 | 23.264 | 63.157 | 1.00 | 14.21 | B |
| ATOM | 2267 | CZ | PHE | B | 117 | 11.163 | 22.305 | 63.833 | 1.00 | 14.33 | B |
| ATOM | 2268 | C | PHE | B | 117 | 6.582 | 21.840 | 61.485 | 1.00 | 16.09 | B |
| ATOM | 2269 | O | PHE | B | 117 | 6.022 | 22.648 | 60.745 | 1.00 | 18.07 | B |
| ATOM | 2270 | N | PRO | B | 118 | 6.536 | 21.953 | 62.820 | 1.00 | 14.95 | B |
| ATOM | 2271 | CD | PRO | B | 118 | 7.011 | 21.012 | 63.850 | 1.00 | 14.55 | B |
| ATOM | 2272 | CA | PRO | B | 118 | 5.785 | 23.053 | 63.428 | 1.00 | 13.85 | B |
| ATOM | 2273 | CB | PRO | B | 118 | 5.567 | 22.571 | 64.859 | 1.00 | 14.86 | B |
| ATOM | 2274 | CG | PRO | B | 118 | 6.801 | 21.798 | 65.132 | 1.00 | 14.98 | B |
| ATOM | 2275 | C | PRO | B | 118 | 6.450 | 24.427 | 63.376 | 1.00 | 12.14 | B |
| ATOM | 2276 | O | PRO | B | 118 | 7.597 | 24.605 | 63.774 | 1.00 | 11.17 | B |
| ATOM | 2277 | N | PHE | B | 119 | 5.698 | 25.389 | 62.864 | 1.00 | 11.54 | B |
| ATOM | 2278 | CA | PHE | B | 119 | 6.127 | 26.775 | 62.766 | 1.00 | 8.50 | B |
| ATOM | 2279 | CB | PHE | B | 119 | 6.206 | 27.225 | 61.304 | 1.00 | 11.00 | B |
| ATOM | 2280 | CG | PHE | B | 119 | 7.460 | 26.810 | 60.600 | 1.00 | 12.44 | B |
| ATOM | 2281 | CD1 | PHE | B | 119 | 8.606 | 27.591 | 60.682 | 1.00 | 12.21 | B |
| ATOM | 2282 | CD2 | PHE | B | 119 | 7.490 | 25.648 | 59.840 | 1.00 | 11.30 | B |
| ATOM | 2283 | CE1 | PHE | B | 119 | 9.768 | 27.223 | 60.012 | 1.00 | 14.47 | B |
| ATOM | 2284 | CE2 | PHE | B | 119 | 8.649 | 25.270 | 59.164 | 1.00 | 12.65 | B |
| ATOM | 2285 | CZ | PHE | B | 119 | 9.790 | 26.062 | 59.252 | 1.00 | 11.82 | B |
| ATOM | 2286 | C | PHE | B | 119 | 5.041 | 27.594 | 63.438 | 1.00 | 7.95 | B |
| ATOM | 2287 | O | PHE | B | 119 | 3.862 | 27.253 | 63.348 | 1.00 | 7.57 | B |
| ATOM | 2288 | N | VAL | B | 120 | 5.438 | 28.653 | 64.131 | 1.00 | 7.57 | B |
| ATOM | 2289 | CA | VAL | B | 120 | 4.480 | 29.559 | 64.749 | 1.00 | 6.76 | B |
| ATOM | 2290 | CB | VAL | B | 120 | 4.573 | 29.567 | 66.284 | 1.00 | 6.90 | B |
| ATOM | 2291 | CG1 | VAL | B | 120 | 3.709 | 30.695 | 66.849 | 1.00 | 6.39 | B |
| ATOM | 2292 | CG2 | VAL | B | 120 | 4.091 | 28.232 | 66.832 | 1.00 | 6.24 | B |
| ATOM | 2293 | C | VAL | B | 120 | 4.922 | 30.895 | 64.183 | 1.00 | 6.55 | B |
| ATOM | 2294 | O | VAL | B | 120 | 6.101 | 31.240 | 64.260 | 1.00 | 8.15 | B |
| ATOM | 2295 | N | ILE | B | 121 | 3.982 | 31.631 | 63.600 | 1.00 | 5.31 | B |
| ATOM | 2296 | CA | ILE | B | 121 | 4.292 | 32.907 | 62.967 | 1.00 | 4.60 | B |
| ATOM | 2297 | CB | ILE | B | 121 | 3.559 | 33.030 | 61.609 | 1.00 | 5.56 | B |
| ATOM | 2298 | CG2 | ILE | B | 121 | 4.058 | 34.259 | 60.850 | 1.00 | 4.58 | B |
| ATOM | 2299 | CG1 | ILE | B | 121 | 3.801 | 31.773 | 60.767 | 1.00 | 7.02 | B |
| ATOM | 2300 | CD1 | ILE | B | 121 | 5.279 | 31.456 | 60.527 | 1.00 | 10.92 | B |
| ATOM | 2301 | C | ILE | B | 121 | 3.927 | 34.108 | 63.827 | 1.00 | 4.76 | B |
| ATOM | 2302 | O | ILE | B | 121 | 2.805 | 34.210 | 64.324 | 1.00 | 4.44 | B |
| ATOM | 2303 | N | LEU | B | 122 | 4.884 | 35.023 | 63.973 | 1.00 | 4.63 | B |
| ATOM | 2304 | CA | LEU | B | 122 | 4.699 | 36.226 | 64.774 | 1.00 | 6.03 | B |
| ATOM | 2305 | CB | LEU | B | 122 | 5.766 | 36.307 | 65.871 | 1.00 | 6.85 | B |
| ATOM | 2306 | CG | LEU | B | 122 | 5.914 | 35.130 | 66.834 | 1.00 | 10.04 | B |
| ATOM | 2307 | CD1 | LEU | B | 122 | 6.897 | 35.517 | 67.941 | 1.00 | 10.00 | B |
| ATOM | 2308 | CD2 | LEU | B | 122 | 4.563 | 34.768 | 67.421 | 1.00 | 13.19 | B |
| ATOM | 2309 | C | LEU | B | 122 | 4.782 | 37.507 | 63.956 | 1.00 | 6.17 | B |
| ATOM | 2310 | O | LEU | B | 122 | 5.790 | 37.760 | 63.290 | 1.00 | 6.53 | B |
| ATOM | 2311 | N | GLY | B | 123 | 3.715 | 38.300 | 64.009 | 1.00 | 5.21 | B |
| ATOM | 2312 | CA | GLY | B | 123 | 3.694 | 39.587 | 63.336 | 1.00 | 5.46 | B |
| ATOM | 2313 | C | GLY | B | 123 | 3.937 | 40.561 | 64.477 | 1.00 | 4.79 | B |
| ATOM | 2314 | O | GLY | B | 123 | 3.008 | 40.903 | 65.203 | 1.00 | 6.75 | B |
| ATOM | 2315 | N | ASN | B | 124 | 5.184 | 41.005 | 64.635 | 1.00 | 2.88 | B |
| ATOM | 2316 | CA | ASN | B | 124 | 5.560 | 41.886 | 65.736 | 1.00 | 3.59 | B |
| ATOM | 2317 | CB | ASN | B | 124 | 6.986 | 41.534 | 66.167 | 1.00 | 5.39 | B |
| ATOM | 2318 | CG | ASN | B | 124 | 7.348 | 42.111 | 67.516 | 1.00 | 5.84 | B |
| ATOM | 2319 | OD1 | ASN | B | 124 | 6.552 | 42.066 | 68.450 | 1.00 | 5.97 | B |
| ATOM | 2320 | ND2 | ASN | B | 124 | 8.562 | 42.632 | 67.633 | 1.00 | 5.99 | B |
| ATOM | 2321 | C | ASN | B | 124 | 5.446 | 43.395 | 65.488 | 1.00 | 5.81 | B |
| ATOM | 2322 | O | ASN | B | 124 | 5.281 | 43.840 | 64.355 | 1.00 | 6.14 | B |
| ATOM | 2323 | N | LYS | B | 125 | 5.536 | 44.164 | 66.575 | 1.00 | 6.02 | B |
| ATOM | 2324 | CA | LYS | B | 125 | 5.453 | 45.624 | 66.546 | 1.00 | 6.58 | B |
| ATOM | 2325 | CB | LYS | B | 125 | 6.470 | 46.198 | 65.555 | 1.00 | 6.91 | B |
| ATOM | 2326 | CG | LYS | B | 125 | 7.904 | 45.751 | 65.801 | 1.00 | 6.84 | B |
| ATOM | 2327 | CD | LYS | B | 125 | 8.881 | 46.646 | 65.056 | 1.00 | 8.56 | B |
| ATOM | 2328 | CE | LYS | B | 125 | 10.313 | 46.186 | 65.242 | 1.00 | 9.77 | B |
| ATOM | 2329 | NZ | LYS | B | 125 | 11.255 | 47.125 | 64.577 | 1.00 | 11.36 | B |
| ATOM | 2330 | C | LYS | B | 125 | 4.058 | 46.137 | 66.194 | 1.00 | 5.75 | B |
| ATOM | 2331 | O | LYS | B | 125 | 3.923 | 47.170 | 65.537 | 1.00 | 6.71 | B |
| ATOM | 2332 | N | ILE | B | 126 | 3.024 | 45.433 | 66.647 | 1.00 | 5.81 | B |
| ATOM | 2333 | CA | ILE | B | 126 | 1.660 | 45.846 | 66.346 | 1.00 | 6.36 | B |
| ATOM | 2334 | CB | ILE | B | 126 | 0.613 | 44.794 | 66.796 | 1.00 | 8.67 | B |
| ATOM | 2335 | CG2 | ILE | B | 126 | 0.831 | 43.485 | 66.057 | 1.00 | 11.76 | B |
| ATOM | 2336 | CG1 | ILE | B | 126 | 0.688 | 44.596 | 68.311 | 1.00 | 9.06 | B |
| ATOM | 2337 | CD1 | ILE | B | 126 | −0.377 | 43.678 | 68.855 | 1.00 | 14.19 | B |
| ATOM | 2338 | C | ILE | B | 126 | 1.292 | 47.174 | 67.001 | 1.00 | 7.50 | B |
| ATOM | 2339 | O | ILE | B | 126 | 0.299 | 47.794 | 66.622 | 1.00 | 8.99 | B |
| ATOM | 2340 | N | ASP | B | 127 | 2.085 | 47.605 | 67.979 | 1.00 | 8.04 | B |
| ATOM | 2341 | CA | ASP | B | 127 | 1.818 | 48.866 | 68.679 | 1.00 | 8.87 | B |
| ATOM | 2342 | CB | ASP | B | 127 | 2.554 | 48.901 | 70.018 | 1.00 | 8.04 | B |
| ATOM | 2343 | CG | ASP | B | 127 | 4.049 | 48.712 | 69.865 | 1.00 | 8.01 | B |
| ATOM | 2344 | OD1 | ASP | B | 127 | 4.472 | 47.595 | 69.495 | 1.00 | 8.67 | B |
| ATOM | 2345 | OD2 | ASP | B | 127 | 4.801 | 49.678 | 70.112 | 1.00 | 9.13 | B |
| ATOM | 2346 | C | ASP | B | 127 | 2.195 | 50.111 | 67.874 | 1.00 | 9.82 | B |
| ATOM | 2347 | O | ASP | B | 127 | 1.793 | 51.227 | 68.220 | 1.00 | 10.25 | B |
| ATOM | 2348 | N | ILE | B | 128 | 2.967 | 49.923 | 66.811 | 1.00 | 8.63 | B |
| ATOM | 2349 | CA | ILE | B | 128 | 3.384 | 51.039 | 65.966 | 1.00 | 8.75 | B |
| ATOM | 2350 | CB | ILE | B | 128 | 4.706 | 50.713 | 65.231 | 1.00 | 7.86 | B |
| ATOM | 2351 | CG2 | ILE | B | 128 | 5.098 | 51.866 | 64.306 | 1.00 | 7.94 | B |
| ATOM | 2352 | CG1 | ILE | B | 128 | 5.806 | 50.458 | 66.264 | 1.00 | 7.68 | B |
| ATOM | 2353 | CD1 | ILE | B | 128 | 7.169 | 50.198 | 65.672 | 1.00 | 6.72 | B |
| ATOM | 2354 | C | ILE | B | 128 | 2.302 | 51.381 | 64.948 | 1.00 | 10.28 | B |
| ATOM | 2355 | O | ILE | B | 128 | 1.689 | 50.491 | 64.351 | 1.00 | 8.67 | B |
| ATOM | 2356 | N | SER | B | 129 | 2.068 | 52.679 | 64.766 | 1.00 | 9.95 | B |
| ATOM | 2357 | CA | SER | B | 129 | 1.056 | 53.166 | 63.836 | 1.00 | 10.04 | B |
| ATOM | 2358 | CB | SER | B | 129 | 0.834 | 54.667 | 64.044 | 1.00 | 12.82 | B |
| ATOM | 2359 | OG | SER | B | 129 | 0.431 | 54.938 | 65.376 | 1.00 | 15.30 | B |
| ATOM | 2360 | C | SER | B | 129 | 1.400 | 52.918 | 62.373 | 1.00 | 8.85 | B |
| ATOM | 2361 | O | SER | B | 129 | 2.568 | 52.777 | 62.004 | 1.00 | 7.32 | B |
| ATOM | 2362 | N | GLU | B | 130 | 0.353 | 52.871 | 61.555 | 1.00 | 8.30 | B |
| ATOM | 2363 | CA | GLU | B | 130 | 0.458 | 52.665 | 60.113 | 1.00 | 8.34 | B |
| ATOM | 2364 | CB | GLU | B | 130 | 1.253 | 53.809 | 59.468 | 1.00 | 9.04 | B |
| ATOM | 2365 | CG | GLU | B | 130 | 0.950 | 55.218 | 60.003 | 1.00 | 10.52 | B |
| ATOM | 2366 | CD | GLU | B | 130 | −0.513 | 55.628 | 59.912 | 1.00 | 12.55 | B |
| ATOM | 2367 | OE1 | GLU | B | 130 | −1.323 | 54.914 | 59.282 | 1.00 | 12.72 | B |
| ATOM | 2368 | OE2 | GLU | B | 130 | −0.851 | 56.692 | 60.477 | 1.00 | 14.14 | B |
| ATOM | 2369 | C | GLU | B | 130 | 1.076 | 51.327 | 59.695 | 1.00 | 8.40 | B |
| ATOM | 2370 | O | GLU | B | 130 | 2.076 | 51.304 | 58.982 | 1.00 | 9.04 | B |
| ATOM | 2371 | N | ARG | B | 131 | 0.475 | 50.221 | 60.131 | 1.00 | 8.92 | B |
| ATOM | 2372 | CA | ARG | B | 131 | 0.955 | 48.882 | 59.776 | 1.00 | 10.67 | B |
| ATOM | 2373 | CB | ARG | B | 131 | −0.048 | 47.811 | 60.186 | 1.00 | 12.93 | B |
| ATOM | 2374 | CG | ARG | B | 131 | −0.499 | 47.822 | 61.607 | 1.00 | 19.23 | B |
| ATOM | 2375 | CD | ARG | B | 131 | −1.585 | 46.774 | 61.769 | 1.00 | 19.84 | B |
| ATOM | 2376 | NE | ARG | B | 131 | −1.240 | 45.526 | 61.089 | 1.00 | 17.67 | B |
| ATOM | 2377 | CZ | ARG | B | 131 | −1.684 | 44.332 | 61.469 | 1.00 | 16.06 | B |
| ATOM | 2378 | NH1 | ARG | B | 131 | −2.485 | 44.237 | 62.520 | 1.00 | 14.89 | B |
| ATOM | 2379 | NH2 | ARG | B | 131 | −1.321 | 43.237 | 60.812 | 1.00 | 11.54 | B |
| ATOM | 2380 | C | ARG | B | 131 | 1.080 | 48.775 | 58.268 | 1.00 | 10.24 | B |
| ATOM | 2381 | O | ARG | B | 131 | 0.258 | 49.332 | 57.543 | 1.00 | 10.22 | B |
| ATOM | 2382 | N | GLN | B | 132 | 2.070 | 48.020 | 57.797 | 1.00 | 9.13 | B |
| ATOM | 2383 | CA | GLN | B | 132 | 2.267 | 47.846 | 56.360 | 1.00 | 9.84 | B |
| ATOM | 2384 | CB | GLN | B | 132 | 3.728 | 48.117 | 55.998 | 1.00 | 10.77 | B |
| ATOM | 2385 | CG | GLN | B | 132 | 4.078 | 49.589 | 56.136 | 1.00 | 12.91 | B |
| ATOM | 2386 | CD | GLN | B | 132 | 3.206 | 50.458 | 55.251 | 1.00 | 13.45 | B |
| ATOM | 2387 | OE1 | GLN | B | 132 | 3.317 | 50.416 | 54.026 | 1.00 | 15.70 | B |
| ATOM | 2388 | NE2 | GLN | B | 132 | 2.319 | 51.238 | 55.864 | 1.00 | 14.74 | B |
| ATOM | 2389 | C | GLN | B | 132 | 1.844 | 46.472 | 55.850 | 1.00 | 9.20 | B |
| ATOM | 2390 | O | GLN | B | 132 | 1.736 | 46.256 | 54.641 | 1.00 | 10.56 | B |
| ATOM | 2391 | N | VAL | B | 133 | 1.603 | 45.550 | 56.775 | 1.00 | 8.06 | B |
| ATOM | 2392 | CA | VAL | B | 133 | 1.173 | 44.200 | 56.428 | 1.00 | 7.78 | B |
| ATOM | 2393 | CB | VAL | B | 133 | 2.150 | 43.138 | 56.974 | 1.00 | 8.29 | B |
| ATOM | 2394 | CG1 | VAL | B | 133 | 1.681 | 41.749 | 56.564 | 1.00 | 8.55 | B |
| ATOM | 2395 | CG2 | VAL | B | 133 | 3.561 | 43.403 | 56.454 | 1.00 | 9.29 | B |
| ATOM | 2396 | C | VAL | B | 133 | −0.188 | 43.988 | 57.081 | 1.00 | 7.06 | B |
| ATOM | 2397 | O | VAL | B | 133 | −0.299 | 44.029 | 58.305 | 1.00 | 7.88 | B |
| ATOM | 2398 | N | SER | B | 134 | −1.223 | 43.765 | 56.276 | 1.00 | 8.19 | B |
| ATOM | 2399 | CA | SER | B | 134 | −2.562 | 43.573 | 56.835 | 1.00 | 8.44 | B |
| ATOM | 2400 | CB | SER | B | 134 | −3.621 | 43.609 | 55.731 | 1.00 | 9.17 | B |
| ATOM | 2401 | OG | SER | B | 134 | −3.554 | 42.449 | 54.924 | 1.00 | 12.63 | B |
| ATOM | 2402 | C | SER | B | 134 | −2.659 | 42.251 | 57.580 | 1.00 | 8.37 | B |
| ATOM | 2403 | O | SER | B | 134 | −1.941 | 41.299 | 57.275 | 1.00 | 7.00 | B |
| ATOM | 2404 | N | THR | B | 135 | −3.553 | 42.191 | 58.561 | 1.00 | 8.33 | B |
| ATOM | 2405 | CA | THR | B | 135 | −3.713 | 40.970 | 59.328 | 1.00 | 7.98 | B |
| ATOM | 2406 | CB | THR | B | 135 | −4.609 | 41.202 | 60.571 | 1.00 | 9.16 | B |
| ATOM | 2407 | OG1 | THR | B | 135 | −4.487 | 40.088 | 61.460 | 1.00 | 7.46 | B |
| ATOM | 2408 | CG2 | THR | B | 135 | −6.066 | 41.367 | 60.168 | 1.00 | 8.04 | B |
| ATOM | 2409 | C | THR | B | 135 | −4.301 | 39.875 | 58.436 | 1.00 | 8.48 | B |
| ATOM | 2410 | O | THR | B | 135 | −4.011 | 38.693 | 58.625 | 1.00 | 7.08 | B |
| ATOM | 2411 | N | GLU | B | 136 | −5.113 | 40.264 | 57.453 | 1.00 | 8.43 | B |
| ATOM | 2412 | CA | GLU | B | 136 | −5.705 | 39.286 | 56.542 | 1.00 | 10.22 | B |
| ATOM | 2413 | CB | GLU | B | 136 | −6.744 | 39.936 | 55.623 | 1.00 | 11.99 | B |
| ATOM | 2414 | CG | GLU | B | 136 | −8.015 | 40.425 | 56.300 | 1.00 | 15.27 | B |
| ATOM | 2415 | CD | GLU | B | 136 | −7.793 | 41.642 | 57.177 | 1.00 | 17.78 | B |
| ATOM | 2416 | OE1 | GLU | B | 136 | −6.822 | 42.395 | 56.932 | 1.00 | 15.88 | B |
| ATOM | 2417 | OE2 | GLU | B | 136 | −8.607 | 41.854 | 58.102 | 1.00 | 18.45 | B |
| ATOM | 2418 | C | GLU | B | 136 | −4.632 | 38.638 | 55.670 | 1.00 | 10.69 | B |
| ATOM | 2419 | O | GLU | B | 136 | −4.633 | 37.425 | 55.464 | 1.00 | 9.31 | B |
| ATOM | 2420 | N | GLU | B | 137 | −3.723 | 39.457 | 55.152 | 1.00 | 10.42 | B |
| ATOM | 2421 | CA | GLU | B | 137 | −2.652 | 38.964 | 54.293 | 1.00 | 11.26 | B |
| ATOM | 2422 | CB | GLU | B | 137 | −1.854 | 40.145 | 53.733 | 1.00 | 15.63 | B |
| ATOM | 2423 | CG | GLU | B | 137 | −0.854 | 39.772 | 52.659 | 1.00 | 22.38 | B |
| ATOM | 2424 | CD | GLU | B | 137 | −0.113 | 40.982 | 52.124 | 1.00 | 23.85 | B |
| ATOM | 2425 | OE1 | GLU | B | 137 | −0.779 | 41.974 | 51.775 | 1.00 | 26.87 | B |
| ATOM | 2426 | OE2 | GLU | B | 137 | 1.133 | 40.940 | 52.050 | 1.00 | 27.97 | B |
| ATOM | 2427 | C | GLU | B | 137 | −1.720 | 38.042 | 55.071 | 1.00 | 9.50 | B |
| ATOM | 2428 | O | GLU | B | 137 | −1.288 | 37.009 | 54.566 | 1.00 | 6.93 | B |
| ATOM | 2429 | N | ALA | B | 138 | −1.411 | 38.427 | 56.304 | 1.00 | 7.93 | B |
| ATOM | 2430 | CA | ALA | B | 138 | −0.525 | 37.632 | 57.144 | 1.00 | 8.01 | B |
| ATOM | 2431 | CB | ALA | B | 138 | −0.230 | 38.373 | 58.449 | 1.00 | 9.01 | B |
| ATOM | 2432 | C | ALA | B | 138 | −1.158 | 36.283 | 57.447 | 1.00 | 7.08 | B |
| ATOM | 2433 | O | ALA | B | 138 | −0.516 | 35.243 | 57.307 | 1.00 | 8.43 | B |
| ATOM | 2434 | N | GLN | B | 139 | −2.419 | 36.310 | 57.867 | 1.00 | 5.89 | B |
| ATOM | 2435 | CA | GLN | B | 139 | −3.140 | 35.085 | 58.205 | 1.00 | 6.63 | B |
| ATOM | 2436 | CB | GLN | B | 139 | −4.515 | 35.425 | 58.782 | 1.00 | 5.57 | B |
| ATOM | 2437 | CG | GLN | B | 139 | −4.449 | 35.953 | 60.210 | 1.00 | 8.07 | B |
| ATOM | 2438 | CD | GLN | B | 139 | −5.800 | 36.366 | 60.750 | 1.00 | 8.48 | B |
| ATOM | 2439 | OE1 | GLN | B | 139 | −6.046 | 37.550 | 61.003 | 1.00 | 12.46 | B |
| ATOM | 2440 | NE2 | GLN | B | 139 | −6.686 | 35.397 | 60.924 | 1.00 | 7.08 | B |
| ATOM | 2441 | C | GLN | B | 139 | −3.288 | 34.168 | 57.000 | 1.00 | 7.46 | B |
| ATOM | 2442 | O | GLN | B | 139 | −3.227 | 32.944 | 57.132 | 1.00 | 8.41 | B |
| ATOM | 2443 | N | ALA | B | 140 | −3.485 | 34.759 | 55.827 | 1.00 | 7.63 | B |
| ATOM | 2444 | CA | ALA | B | 140 | −3.629 | 33.982 | 54.604 | 1.00 | 7.82 | B |
| ATOM | 2445 | CB | ALA | B | 140 | −3.978 | 34.895 | 53.445 | 1.00 | 9.09 | B |
| ATOM | 2446 | C | ALA | B | 140 | −2.348 | 33.210 | 54.297 | 1.00 | 9.51 | B |
| ATOM | 2447 | O | ALA | B | 140 | −2.402 | 32.058 | 53.859 | 1.00 | 10.52 | B |
| ATOM | 2448 | N | TRP | B | 141 | −1.199 | 33.844 | 54.513 | 1.00 | 8.84 | B |
| ATOM | 2449 | CA | TRP | B | 141 | 0.083 | 33.188 | 54.263 | 1.00 | 9.42 | B |
| ATOM | 2450 | CB | TRP | B | 141 | 1.249 | 34.147 | 54.520 | 1.00 | 10.72 | B |
| ATOM | 2451 | CG | TRP | B | 141 | 2.582 | 33.577 | 54.112 | 1.00 | 11.44 | B |
| ATOM | 2452 | CD2 | TRP | B | 141 | 3.453 | 32.769 | 54.913 | 1.00 | 11.26 | B |
| ATOM | 2453 | CE2 | TRP | B | 141 | 4.546 | 32.395 | 54.097 | 1.00 | 12.66 | B |
| ATOM | 2454 | CE3 | TRP | B | 141 | 3.414 | 32.319 | 56.241 | 1.00 | 11.67 | B |
| ATOM | 2455 | CD1 | TRP | B | 141 | 3.169 | 33.662 | 52.876 | 1.00 | 14.07 | B |
| ATOM | 2456 | NE1 | TRP | B | 141 | 4.348 | 32.953 | 52.861 | 1.00 | 12.65 | B |
| ATOM | 2457 | CZ2 | TRP | B | 141 | 5.592 | 31.592 | 54.566 | 1.00 | 12.99 | B |
| ATOM | 2458 | CZ3 | TRP | B | 141 | 4.454 | 31.519 | 56.708 | 1.00 | 11.79 | B |
| ATOM | 2459 | CH2 | TRP | B | 141 | 5.528 | 31.164 | 55.869 | 1.00 | 11.62 | B |
| ATOM | 2460 | C | TRP | B | 141 | 0.215 | 31.990 | 55.196 | 1.00 | 10.07 | B |
| ATOM | 2461 | O | TRP | B | 141 | 0.601 | 30.896 | 54.775 | 1.00 | 9.94 | B |
| ATOM | 2462 | N | CYS | B | 142 | −0.113 | 32.204 | 56.467 | 1.00 | 9.16 | B |
| ATOM | 2463 | CA | CYS | B | 142 | −0.024 | 31.146 | 57.463 | 1.00 | 9.99 | B |
| ATOM | 2464 | CB | CYS | B | 142 | −0.422 | 31.674 | 58.844 | 1.00 | 11.04 | B |
| ATOM | 2465 | SG | CYS | B | 142 | 0.721 | 32.880 | 59.538 | 1.00 | 10.54 | B |
| ATOM | 2466 | C | CYS | B | 142 | −0.920 | 29.978 | 57.099 | 1.00 | 11.08 | B |
| ATOM | 2467 | O | CYS | B | 142 | −0.538 | 28.817 | 57.252 | 1.00 | 8.82 | B |
| ATOM | 2468 | N | ARG | B | 143 | −2.110 | 30.294 | 56.604 | 1.00 | 10.98 | B |
| ATOM | 2469 | CA | ARG | B | 143 | −3.076 | 29.271 | 56.236 | 1.00 | 15.56 | B |
| ATOM | 2470 | CB | ARG | B | 143 | −4.418 | 29.932 | 55.888 | 1.00 | 16.02 | B |
| ATOM | 2471 | CG | ARG | B | 143 | −5.565 | 28.962 | 55.647 | 1.00 | 18.84 | B |
| ATOM | 2472 | CD | ARG | B | 143 | −5.963 | 28.231 | 56.919 | 1.00 | 18.92 | B |
| ATOM | 2473 | NE | ARG | B | 143 | −7.012 | 27.247 | 56.673 | 1.00 | 20.86 | B |
| ATOM | 2474 | CZ | ARG | B | 143 | −8.280 | 27.546 | 56.408 | 1.00 | 22.11 | B |
| ATOM | 2475 | NH1 | ARG | B | 143 | −8.673 | 28.812 | 56.356 | 1.00 | 21.91 | B |
| ATOM | 2476 | NH2 | ARG | B | 143 | −9.157 | 26.574 | 56.181 | 1.00 | 24.11 | B |
| ATOM | 2477 | C | ARG | B | 143 | −2.606 | 28.394 | 55.075 | 1.00 | 16.72 | B |
| ATOM | 2478 | O | ARG | B | 143 | −2.577 | 27.171 | 55.195 | 1.00 | 17.72 | B |
| ATOM | 2479 | N | ASP | B | 144 | −2.214 | 29.004 | 53.960 | 1.00 | 18.51 | B |
| ATOM | 2480 | CA | ASP | B | 144 | −1.798 | 28.213 | 52.809 | 1.00 | 19.40 | B |
| ATOM | 2481 | CB | ASP | B | 144 | −2.225 | 28.902 | 51.506 | 1.00 | 24.74 | B |
| ATOM | 2482 | CG | ASP | B | 144 | −1.924 | 30.380 | 51.493 | 1.00 | 26.32 | B |
| ATOM | 2483 | OD1 | ASP | B | 144 | −0.737 | 30.747 | 51.609 | 1.00 | 31.68 | B |
| ATOM | 2484 | OD2 | ASP | B | 144 | −2.881 | 31.176 | 51.359 | 1.00 | 29.28 | B |
| ATOM | 2485 | C | ASP | B | 144 | −0.336 | 27.792 | 52.717 | 1.00 | 18.84 | B |
| ATOM | 2486 | O | ASP | B | 144 | 0.104 | 27.308 | 51.675 | 1.00 | 20.43 | B |
| ATOM | 2487 | N | ASN | B | 145 | 0.420 | 27.956 | 53.795 | 1.00 | 16.09 | B |
| ATOM | 2488 | CA | ASN | B | 145 | 1.817 | 27.537 | 53.781 | 1.00 | 15.85 | B |
| ATOM | 2489 | CB | ASN | B | 145 | 2.756 | 28.743 | 53.884 | 1.00 | 16.18 | B |
| ATOM | 2490 | CG | ASN | B | 145 | 2.792 | 29.557 | 52.609 | 1.00 | 17.07 | B |
| ATOM | 2491 | OD1 | ASN | B | 145 | 1.965 | 30.444 | 52.398 | 1.00 | 18.85 | B |
| ATOM | 2492 | ND2 | ASN | B | 145 | 3.743 | 29.240 | 51.734 | 1.00 | 18.68 | B |
| ATOM | 2493 | C | ASN | B | 145 | 2.111 | 26.548 | 54.897 | 1.00 | 15.32 | B |
| ATOM | 2494 | O | ASN | B | 145 | 3.264 | 26.349 | 55.269 | 1.00 | 16.99 | B |
| ATOM | 2495 | N | GLY | B | 146 | 1.060 | 25.926 | 55.426 | 1.00 | 15.04 | B |
| ATOM | 2496 | CA | GLY | B | 146 | 1.238 | 24.951 | 56.485 | 1.00 | 13.71 | B |
| ATOM | 2497 | C | GLY | B | 146 | 0.223 | 25.015 | 57.612 | 1.00 | 13.99 | B |
| ATOM | 2498 | O | GLY | B | 146 | 0.302 | 24.228 | 58.555 | 1.00 | 14.51 | B |
| ATOM | 2499 | N | ASP | B | 147 | −0.728 | 25.942 | 57.520 | 1.00 | 13.75 | B |
| ATOM | 2500 | CA | ASP | B | 147 | −1.754 | 26.106 | 58.553 | 1.00 | 13.67 | B |
| ATOM | 2501 | CB | ASP | B | 147 | −2.657 | 24.874 | 58.612 | 1.00 | 17.77 | B |
| ATOM | 2502 | CG | ASP | B | 147 | −3.562 | 24.764 | 57.405 | 1.00 | 21.81 | B |
| ATOM | 2503 | OD1 | ASP | B | 147 | −4.364 | 25.695 | 57.182 | 1.00 | 23.77 | B |
| ATOM | 2504 | OD2 | ASP | B | 147 | −3.470 | 23.751 | 56.680 | 1.00 | 26.77 | B |
| ATOM | 2505 | C | ASP | B | 147 | −1.111 | 26.342 | 59.914 | 1.00 | 12.71 | B |
| ATOM | 2506 | O | ASP | B | 147 | −1.428 | 25.678 | 60.903 | 1.00 | 11.48 | B |
| ATOM | 2507 | N | TYR | B | 148 | −0.208 | 27.310 | 59.955 | 1.00 | 9.11 | B |
| ATOM | 2508 | CA | TYR | B | 148 | 0.498 | 27.631 | 61.180 | 1.00 | 9.00 | B |
| ATOM | 2509 | CB | TYR | B | 148 | 1.853 | 28.268 | 60.864 | 1.00 | 8.98 | B |
| ATOM | 2510 | CG | TYR | B | 148 | 2.731 | 27.511 | 59.894 | 1.00 | 8.31 | B |
| ATOM | 2511 | CD1 | TYR | B | 148 | 3.019 | 26.162 | 60.084 | 1.00 | 8.65 | B |
| ATOM | 2512 | CE1 | TYR | B | 148 | 3.900 | 25.485 | 59.235 | 1.00 | 8.99 | B |
| ATOM | 2513 | CD2 | TYR | B | 148 | 3.337 | 28.173 | 58.826 | 1.00 | 9.48 | B |
| ATOM | 2514 | CE2 | TYR | B | 148 | 4.221 | 27.508 | 57.973 | 1.00 | 9.61 | B |
| ATOM | 2515 | CZ | TYR | B | 148 | 4.498 | 26.170 | 58.186 | 1.00 | 10.42 | B |
| ATOM | 2516 | OH | TYR | B | 148 | 5.395 | 25.528 | 57.362 | 1.00 | 8.97 | B |
| ATOM | 2517 | C | TYR | B | 148 | −0.260 | 28.614 | 62.053 | 1.00 | 8.48 | B |
| ATOM | 2518 | O | TYR | B | 148 | −0.998 | 29.469 | 61.556 | 1.00 | 8.16 | B |
| ATOM | 2519 | N | PRO | B | 149 | −0.099 | 28.497 | 63.377 | 1.00 | 6.86 | B |
| ATOM | 2520 | CD | PRO | B | 149 | 0.457 | 27.381 | 64.161 | 1.00 | 7.29 | B |
| ATOM | 2521 | CA | PRO | B | 149 | −0.799 | 29.453 | 64.237 | 1.00 | 6.32 | B |
| ATOM | 2522 | CB | PRO | B | 149 | −0.581 | 28.893 | 65.643 | 1.00 | 8.96 | B |
| ATOM | 2523 | CG | PRO | B | 149 | 0.635 | 28.011 | 65.507 | 1.00 | 11.23 | B |
| ATOM | 2524 | C | PRO | B | 149 | −0.117 | 30.806 | 64.020 | 1.00 | 7.91 | B |
| ATOM | 2525 | O | PRO | B | 149 | 1.093 | 30.868 | 63.751 | 1.00 | 6.29 | B |
| ATOM | 2526 | N | TYR | B | 150 | −0.892 | 31.878 | 64.132 | 1.00 | 5.65 | B |
| ATOM | 2527 | CA | TYR | B | 150 | −0.385 | 33.227 | 63.916 | 1.00 | 5.51 | B |
| ATOM | 2528 | CB | TYR | B | 150 | −1.003 | 33.806 | 62.639 | 1.00 | 6.14 | B |
| ATOM | 2529 | CG | TYR | B | 150 | −0.677 | 35.264 | 62.382 | 1.00 | 7.06 | B |
| ATOM | 2530 | CD1 | TYR | B | 150 | 0.630 | 35.670 | 62.113 | 1.00 | 6.44 | B |
| ATOM | 2531 | CE1 | TYR | B | 150 | 0.934 | 37.011 | 61.868 | 1.00 | 7.42 | B |
| ATOM | 2532 | CD2 | TYR | B | 150 | −1.679 | 36.238 | 62.403 | 1.00 | 6.82 | B |
| ATOM | 2533 | CE2 | TYR | B | 150 | −1.388 | 37.581 | 62.158 | 1.00 | 6.96 | B |
| ATOM | 2534 | CZ | TYR | B | 150 | −0.081 | 37.959 | 61.892 | 1.00 | 8.41 | B |
| ATOM | 2535 | OH | TYR | B | 150 | 0.213 | 39.282 | 61.654 | 1.00 | 7.55 | B |
| ATOM | 2536 | C | TYR | B | 150 | −0.710 | 34.146 | 65.085 | 1.00 | 5.97 | B |
| ATOM | 2537 | O | TYR | B | 150 | −1.839 | 34.158 | 65.573 | 1.00 | 6.31 | B |
| ATOM | 2538 | N | PHE | B | 151 | 0.280 | 34.919 | 65.524 | 1.00 | 5.09 | B |
| ATOM | 2539 | CA | PHE | B | 151 | 0.081 | 35.861 | 66.625 | 1.00 | 5.63 | B |
| ATOM | 2540 | CB | PHE | B | 151 | 0.813 | 35.406 | 67.890 | 1.00 | 5.01 | B |
| ATOM | 2541 | CG | PHE | B | 151 | 0.311 | 34.113 | 68.454 | 1.00 | 5.22 | B |
| ATOM | 2542 | CD1 | PHE | B | 151 | 0.814 | 32.902 | 68.000 | 1.00 | 7.78 | B |
| ATOM | 2543 | CD2 | PHE | B | 151 | −0.677 | 34.107 | 69.433 | 1.00 | 7.81 | B |
| ATOM | 2544 | CE1 | PHE | B | 151 | 0.344 | 31.697 | 68.511 | 1.00 | 8.40 | B |
| ATOM | 2545 | CE2 | PHE | B | 151 | −1.156 | 32.906 | 69.952 | 1.00 | 9.02 | B |
| ATOM | 2546 | CZ | PHE | B | 151 | −0.642 | 31.699 | 69.488 | 1.00 | 8.18 | B |
| ATOM | 2547 | C | PHE | B | 151 | 0.594 | 37.249 | 66.275 | 1.00 | 5.03 | B |
| ATOM | 2548 | O | PHE | B | 151 | 1.695 | 37.394 | 65.745 | 1.00 | 6.88 | B |
| ATOM | 2549 | N | GLU | B | 152 | −0.212 | 38.264 | 66.567 | 1.00 | 7.30 | B |
| ATOM | 2550 | CA | GLU | B | 152 | 0.190 | 39.646 | 66.344 | 1.00 | 7.16 | B |
| ATOM | 2551 | CB | GLU | B | 152 | −1.012 | 40.500 | 65.936 | 1.00 | 10.30 | B |
| ATOM | 2552 | CG | GLU | B | 152 | −1.724 | 39.964 | 64.702 | 1.00 | 11.36 | B |
| ATOM | 2553 | CD | GLU | B | 152 | −2.117 | 41.047 | 63.719 | 1.00 | 11.11 | B |
| ATOM | 2554 | OE1 | GLU | B | 152 | −2.699 | 42.064 | 64.152 | 1.00 | 12.10 | B |
| ATOM | 2555 | OE2 | GLU | B | 152 | −1.855 | 40.872 | 62.508 | 1.00 | 8.85 | B |
| ATOM | 2556 | C | GLU | B | 152 | 0.712 | 40.040 | 67.719 | 1.00 | 8.33 | B |
| ATOM | 2557 | O | GLU | B | 152 | −0.041 | 40.107 | 68.687 | 1.00 | 8.61 | B |
| ATOM | 2558 | N | THR | B | 153 | 2.012 | 40.288 | 67.796 | 1.00 | 7.73 | B |
| ATOM | 2559 | CA | THR | B | 153 | 2.656 | 40.586 | 69.063 | 1.00 | 7.67 | B |
| ATOM | 2560 | CB | THR | B | 153 | 3.805 | 39.604 | 69.309 | 1.00 | 8.96 | B |
| ATOM | 2561 | OG1 | THR | B | 153 | 4.818 | 39.816 | 68.314 | 1.00 | 8.48 | B |
| ATOM | 2562 | CG2 | THR | B | 153 | 3.311 | 38.162 | 69.220 | 1.00 | 7.61 | B |
| ATOM | 2563 | C | THR | B | 153 | 3.256 | 41.971 | 69.183 | 1.00 | 6.77 | B |
| ATOM | 2564 | O | THR | B | 153 | 3.366 | 42.709 | 68.207 | 1.00 | 7.22 | B |
| ATOM | 2565 | N | SER | B | 154 | 3.648 | 42.299 | 70.408 | 1.00 | 6.17 | B |
| ATOM | 2566 | CA | SER | B | 154 | 4.314 | 43.554 | 70.716 | 1.00 | 7.23 | B |
| ATOM | 2567 | CB | SER | B | 154 | 3.322 | 44.650 | 71.096 | 1.00 | 7.82 | B |
| ATOM | 2568 | OG | SER | B | 154 | 4.026 | 45.791 | 71.563 | 1.00 | 9.05 | B |
| ATOM | 2569 | C | SER | B | 154 | 5.237 | 43.311 | 71.897 | 1.00 | 7.83 | B |
| ATOM | 2570 | O | SER | B | 154 | 4.770 | 43.070 | 73.015 | 1.00 | 8.83 | B |
| ATOM | 2571 | N | ALA | B | 155 | 6.542 | 43.359 | 71.653 | 1.00 | 8.49 | B |
| ATOM | 2572 | CA | ALA | B | 155 | 7.506 | 43.166 | 72.728 | 1.00 | 8.89 | B |
| ATOM | 2573 | CB | ALA | B | 155 | 8.922 | 43.101 | 72.165 | 1.00 | 7.54 | B |
| ATOM | 2574 | C | ALA | B | 155 | 7.374 | 44.350 | 73.685 | 1.00 | 9.26 | B |
| ATOM | 2575 | O | ALA | B | 155 | 7.630 | 44.227 | 74.879 | 1.00 | 8.40 | B |
| ATOM | 2576 | N | LYS | B | 156 | 6.959 | 45.496 | 73.152 | 1.00 | 9.55 | B |
| ATOM | 2577 | CA | LYS | B | 156 | 6.797 | 46.700 | 73.962 | 1.00 | 10.89 | B |
| ATOM | 2578 | CB | LYS | B | 156 | 6.492 | 47.906 | 73.059 | 1.00 | 11.96 | B |
| ATOM | 2579 | CG | LYS | B | 156 | 6.224 | 49.217 | 73.806 | 1.00 | 15.93 | B |
| ATOM | 2580 | CD | LYS | B | 156 | 4.728 | 49.453 | 73.985 | 1.00 | 21.82 | B |
| ATOM | 2581 | CE | LYS | B | 156 | 4.432 | 50.846 | 74.536 | 1.00 | 24.22 | B |
| ATOM | 2582 | NZ | LYS | B | 156 | 2.970 | 51.159 | 74.516 | 1.00 | 23.74 | B |
| ATOM | 2583 | C | LYS | B | 156 | 5.729 | 46.578 | 75.050 | 1.00 | 11.02 | B |
| ATOM | 2584 | O | LYS | B | 156 | 6.007 | 46.847 | 76.220 | 1.00 | 10.62 | B |
| ATOM | 2585 | N | ASP | B | 157 | 4.512 | 46.186 | 74.685 | 1.00 | 12.49 | B |
| ATOM | 2586 | CA | ASP | B | 157 | 3.464 | 46.060 | 75.697 | 1.00 | 14.53 | B |
| ATOM | 2587 | CB | ASP | B | 157 | 2.189 | 46.795 | 75.271 | 1.00 | 19.72 | B |
| ATOM | 2588 | CG | ASP | B | 157 | 1.720 | 46.401 | 73.901 | 1.00 | 25.21 | B |
| ATOM | 2589 | OD1 | ASP | B | 157 | 1.746 | 45.192 | 73.595 | 1.00 | 28.51 | B |
| ATOM | 2590 | OD2 | ASP | B | 157 | 1.313 | 47.304 | 73.133 | 1.00 | 29.90 | B |
| ATOM | 2591 | C | ASP | B | 157 | 3.126 | 44.627 | 76.091 | 1.00 | 12.91 | B |
| ATOM | 2592 | O | ASP | B | 157 | 2.171 | 44.399 | 76.829 | 1.00 | 14.47 | B |
| ATOM | 2593 | N | ALA | B | 158 | 3.917 | 43.677 | 75.594 | 1.00 | 12.04 | B |
| ATOM | 2594 | CA | ALA | B | 158 | 3.771 | 42.250 | 75.898 | 1.00 | 11.66 | B |
| ATOM | 2595 | CB | ALA | B | 158 | 3.723 | 42.046 | 77.421 | 1.00 | 13.66 | B |
| ATOM | 2596 | C | ALA | B | 158 | 2.601 | 41.519 | 75.248 | 1.00 | 10.64 | B |
| ATOM | 2597 | O | ALA | B | 158 | 2.392 | 40.336 | 75.506 | 1.00 | 10.51 | B |
| ATOM | 2598 | N | THR | B | 159 | 1.852 | 42.209 | 74.398 | 1.00 | 8.81 | B |
| ATOM | 2599 | CA | THR | B | 159 | 0.705 | 41.601 | 73.742 | 1.00 | 9.95 | B |
| ATOM | 2600 | CB | THR | B | 159 | 0.079 | 42.577 | 72.731 | 1.00 | 11.71 | B |
| ATOM | 2601 | OG1 | THR | B | 159 | −0.227 | 43.812 | 73.391 | 1.00 | 13.20 | B |
| ATOM | 2602 | CG2 | THR | B | 159 | −1.198 | 41.993 | 72.152 | 1.00 | 10.36 | B |
| ATOM | 2603 | C | THR | B | 159 | 1.026 | 40.290 | 73.020 | 1.00 | 9.21 | B |
| ATOM | 2604 | O | THR | B | 159 | 1.877 | 40.248 | 72.126 | 1.00 | 7.63 | B |
| ATOM | 2605 | N | ASN | B | 160 | 0.345 | 39.222 | 73.432 | 1.00 | 8.82 | B |
| ATOM | 2606 | CA | ASN | B | 160 | 0.500 | 37.900 | 72.828 | 1.00 | 8.36 | B |
| ATOM | 2607 | CB | ASN | B | 160 | 0.067 | 37.955 | 71.363 | 1.00 | 10.63 | B |
| ATOM | 2608 | CG | ASN | B | 160 | −1.434 | 38.066 | 71.201 | 1.00 | 14.04 | B |
| ATOM | 2609 | OD1 | ASN | B | 160 | −1.922 | 38.565 | 70.186 | 1.00 | 13.44 | B |
| ATOM | 2610 | ND2 | ASN | B | 160 | −2.179 | 37.586 | 72.196 | 1.00 | 11.28 | B |
| ATOM | 2611 | C | ASN | B | 160 | 1.887 | 37.267 | 72.905 | 1.00 | 8.14 | B |
| ATOM | 2612 | O | ASN | B | 160 | 2.121 | 36.233 | 72.286 | 1.00 | 8.24 | B |
| ATOM | 2613 | N | VAL | B | 161 | 2.801 | 37.862 | 73.663 | 1.00 | 7.78 | B |
| ATOM | 2614 | CA | VAL | B | 161 | 4.147 | 37.306 | 73.764 | 1.00 | 8.73 | B |
| ATOM | 2615 | CB | VAL | B | 161 | 5.099 | 38.269 | 74.502 | 1.00 | 9.57 | B |
| ATOM | 2616 | CG1 | VAL | B | 161 | 6.472 | 37.622 | 74.660 | 1.00 | 10.54 | B |
| ATOM | 2617 | CG2 | VAL | B | 161 | 5.226 | 39.572 | 73.711 | 1.00 | 11.47 | B |
| ATOM | 2618 | C | VAL | B | 161 | 4.191 | 35.937 | 74.445 | 1.00 | 7.62 | B |
| ATOM | 2619 | O | VAL | B | 161 | 4.728 | 34.981 | 73.888 | 1.00 | 6.80 | B |
| ATOM | 2620 | N | ALA | B | 162 | 3.636 | 35.837 | 75.647 | 1.00 | 8.65 | B |
| ATOM | 2621 | CA | ALA | B | 162 | 3.633 | 34.561 | 76.352 | 1.00 | 9.39 | B |
| ATOM | 2622 | CB | ALA | B | 162 | 3.036 | 34.728 | 77.745 | 1.00 | 11.45 | B |
| ATOM | 2623 | C | ALA | B | 162 | 2.831 | 33.531 | 75.563 | 1.00 | 8.80 | B |
| ATOM | 2624 | O | ALA | B | 162 | 3.211 | 32.362 | 75.469 | 1.00 | 7.77 | B |
| ATOM | 2625 | N | ALA | B | 163 | 1.718 | 33.971 | 74.988 | 1.00 | 8.53 | B |
| ATOM | 2626 | CA | ALA | B | 163 | 0.868 | 33.074 | 74.224 | 1.00 | 8.01 | B |
| ATOM | 2627 | CB | ALA | B | 163 | −0.366 | 33.819 | 73.732 | 1.00 | 10.04 | B |
| ATOM | 2628 | C | ALA | B | 163 | 1.605 | 32.450 | 73.046 | 1.00 | 7.80 | B |
| ATOM | 2629 | O | ALA | B | 163 | 1.439 | 31.265 | 72.766 | 1.00 | 8.00 | B |
| ATOM | 2630 | N | ALA | B | 164 | 2.420 | 33.247 | 72.359 | 1.00 | 7.59 | B |
| ATOM | 2631 | CA | ALA | B | 164 | 3.167 | 32.761 | 71.200 | 1.00 | 7.26 | B |
| ATOM | 2632 | CB | ALA | B | 164 | 3.833 | 33.925 | 70.491 | 1.00 | 7.62 | B |
| ATOM | 2633 | C | ALA | B | 164 | 4.214 | 31.715 | 71.565 | 1.00 | 6.80 | B |
| ATOM | 2634 | O | ALA | B | 164 | 4.334 | 30.685 | 70.896 | 1.00 | 7.89 | B |
| ATOM | 2635 | N | PHE | B | 165 | 4.980 | 31.981 | 72.616 | 1.00 | 5.40 | B |
| ATOM | 2636 | CA | PHE | B | 165 | 6.007 | 31.045 | 73.048 | 1.00 | 8.07 | B |
| ATOM | 2637 | CB | PHE | B | 165 | 6.864 | 31.682 | 74.140 | 1.00 | 9.62 | B |
| ATOM | 2638 | CG | PHE | B | 165 | 7.962 | 32.550 | 73.610 | 1.00 | 10.93 | B |
| ATOM | 2639 | CD1 | PHE | B | 165 | 9.132 | 31.982 | 73.120 | 1.00 | 10.70 | B |
| ATOM | 2640 | CD2 | PHE | B | 165 | 7.831 | 33.934 | 73.601 | 1.00 | 11.47 | B |
| ATOM | 2641 | CE1 | PHE | B | 165 | 10.162 | 32.783 | 72.630 | 1.00 | 11.69 | B |
| ATOM | 2642 | CE2 | PHE | B | 165 | 8.856 | 34.744 | 73.111 | 1.00 | 13.90 | B |
| ATOM | 2643 | CZ | PHE | B | 165 | 10.025 | 34.164 | 72.626 | 1.00 | 13.02 | B |
| ATOM | 2644 | C | PHE | B | 165 | 5.382 | 29.752 | 73.553 | 1.00 | 8.58 | B |
| ATOM | 2645 | O | PHE | B | 165 | 5.893 | 28.659 | 73.298 | 1.00 | 8.30 | B |
| ATOM | 2646 | N | GLU | B | 166 | 4.269 | 29.884 | 74.264 | 1.00 | 10.98 | B |
| ATOM | 2647 | CA | GLU | B | 166 | 3.564 | 28.728 | 74.797 | 1.00 | 11.68 | B |
| ATOM | 2648 | CB | GLU | B | 166 | 2.446 | 29.190 | 75.730 | 1.00 | 14.22 | B |
| ATOM | 2649 | CG | GLU | B | 166 | 2.952 | 29.767 | 77.042 | 1.00 | 17.64 | B |
| ATOM | 2650 | CD | GLU | B | 166 | 1.832 | 30.305 | 77.910 | 1.00 | 21.43 | B |
| ATOM | 2651 | OE1 | GLU | B | 166 | 0.819 | 29.597 | 78.077 | 1.00 | 25.16 | B |
| ATOM | 2652 | OE2 | GLU | B | 166 | 1.966 | 31.431 | 78.431 | 1.00 | 24.67 | B |
| ATOM | 2653 | C | GLU | B | 166 | 2.995 | 27.871 | 73.671 | 1.00 | 10.41 | B |
| ATOM | 2654 | O | GLU | B | 166 | 3.016 | 26.640 | 73.739 | 1.00 | 9.67 | B |
| ATOM | 2655 | N | GLU | B | 167 | 2.491 | 28.524 | 72.632 | 1.00 | 8.99 | B |
| ATOM | 2656 | CA | GLU | B | 167 | 1.930 | 27.809 | 71.492 | 1.00 | 8.32 | B |
| ATOM | 2657 | CB | GLU | B | 167 | 1.317 | 28.800 | 70.498 | 1.00 | 8.63 | B |
| ATOM | 2658 | CG | GLU | B | 167 | 0.696 | 28.163 | 69.258 | 1.00 | 10.89 | B |
| ATOM | 2659 | CD | GLU | B | 167 | −0.346 | 27.109 | 69.582 | 1.00 | 15.29 | B |
| ATOM | 2660 | OE1 | GLU | B | 167 | −0.971 | 27.190 | 70.660 | 1.00 | 15.75 | B |
| ATOM | 2661 | OE2 | GLU | B | 167 | −0.551 | 26.205 | 68.743 | 1.00 | 16.38 | B |
| ATOM | 2662 | C | GLU | B | 167 | 3.016 | 26.991 | 70.808 | 1.00 | 8.35 | B |
| ATOM | 2663 | O | GLU | B | 167 | 2.753 | 25.896 | 70.312 | 1.00 | 7.38 | B |
| ATOM | 2664 | N | ALA | B | 168 | 4.237 | 27.522 | 70.787 | 1.00 | 7.32 | B |
| ATOM | 2665 | CA | ALA | B | 168 | 5.355 | 26.820 | 70.163 | 1.00 | 8.91 | B |
| ATOM | 2666 | CB | ALA | B | 168 | 6.647 | 27.630 | 70.315 | 1.00 | 10.30 | B |
| ATOM | 2667 | C | ALA | B | 168 | 5.510 | 25.446 | 70.808 | 1.00 | 9.23 | B |
| ATOM | 2668 | O | ALA | B | 168 | 5.647 | 24.433 | 70.113 | 1.00 | 7.36 | B |
| ATOM | 2669 | N | VAL | B | 169 | 5.471 | 25.413 | 72.138 | 1.00 | 8.71 | B |
| ATOM | 2670 | CA | VAL | B | 169 | 5.590 | 24.157 | 72.872 | 1.00 | 10.23 | B |
| ATOM | 2671 | CB | VAL | B | 169 | 5.714 | 24.412 | 74.398 | 1.00 | 10.38 | B |
| ATOM | 2672 | CG1 | VAL | B | 169 | 5.691 | 23.096 | 75.155 | 1.00 | 11.92 | B |
| ATOM | 2673 | CG2 | VAL | B | 169 | 7.016 | 25.146 | 74.692 | 1.00 | 11.34 | B |
| ATOM | 2674 | C | VAL | B | 169 | 4.374 | 23.271 | 72.597 | 1.00 | 10.31 | B |
| ATOM | 2675 | O | VAL | B | 169 | 4.501 | 22.059 | 72.446 | 1.00 | 10.84 | B |
| ATOM | 2676 | N | ARG | B | 170 | 3.198 | 23.886 | 72.517 | 1.00 | 9.61 | B |
| ATOM | 2677 | CA | ARG | B | 170 | 1.965 | 23.154 | 72.251 | 1.00 | 9.91 | B |
| ATOM | 2678 | CB | ARG | B | 170 | 0.765 | 24.116 | 72.282 | 1.00 | 12.33 | B |
| ATOM | 2679 | CG | ARG | B | 170 | −0.587 | 23.453 | 72.042 | 1.00 | 13.81 | B |
| ATOM | 2680 | CD | ARG | B | 170 | −1.752 | 24.384 | 72.407 | 1.00 | 13.77 | B |
| ATOM | 2681 | NE | ARG | B | 170 | −1.773 | 24.695 | 73.834 | 1.00 | 13.20 | B |
| ATOM | 2682 | CZ | ARG | B | 170 | −1.519 | 25.897 | 74.345 | 1.00 | 15.48 | B |
| ATOM | 2683 | NH1 | ARG | B | 170 | −1.230 | 26.918 | 73.545 | 1.00 | 14.15 | B |
| ATOM | 2684 | NH2 | ARG | B | 170 | −1.529 | 26.075 | 75.660 | 1.00 | 16.86 | B |
| ATOM | 2685 | C | ARG | B | 170 | 2.043 | 22.435 | 70.906 | 1.00 | 10.46 | B |
| ATOM | 2686 | O | ARG | B | 170 | 1.638 | 21.280 | 70.789 | 1.00 | 11.15 | B |
| ATOM | 2687 | N | ARG | B | 171 | 2.568 | 23.117 | 69.892 | 1.00 | 10.12 | B |
| ATOM | 2688 | CA | ARG | B | 171 | 2.703 | 22.519 | 68.567 | 1.00 | 10.89 | B |
| ATOM | 2689 | CB | ARG | B | 171 | 3.168 | 23.572 | 67.554 | 1.00 | 10.45 | B |
| ATOM | 2690 | CG | ARG | B | 171 | 2.036 | 24.430 | 67.012 | 1.00 | 15.15 | B |
| ATOM | 2691 | CD | ARG | B | 171 | 1.128 | 23.590 | 66.117 | 1.00 | 18.84 | B |
| ATOM | 2692 | NE | ARG | B | 171 | −0.057 | 24.314 | 65.675 | 1.00 | 26.06 | B |
| ATOM | 2693 | CZ | ARG | B | 171 | −0.942 | 23.824 | 64.812 | 1.00 | 28.92 | B |
| ATOM | 2694 | NH1 | ARG | B | 171 | −0.766 | 22.610 | 64.304 | 1.00 | 31.79 | B |
| ATOM | 2695 | NH2 | ARG | B | 171 | −1.998 | 24.545 | 64.451 | 1.00 | 30.65 | B |
| ATOM | 2696 | C | ARG | B | 171 | 3.670 | 21.337 | 68.580 | 1.00 | 10.99 | B |
| ATOM | 2697 | O | ARG | B | 171 | 3.463 | 20.354 | 67.870 | 1.00 | 11.14 | B |
| ATOM | 2698 | N | VAL | B | 172 | 4.722 | 21.425 | 69.388 | 1.00 | 12.41 | B |
| ATOM | 2699 | CA | VAL | B | 172 | 5.678 | 20.324 | 69.476 | 1.00 | 14.04 | B |
| ATOM | 2700 | CB | VAL | B | 172 | 6.877 | 20.682 | 70.386 | 1.00 | 14.10 | B |
| ATOM | 2701 | CG1 | VAL | B | 172 | 7.812 | 19.484 | 70.516 | 1.00 | 16.28 | B |
| ATOM | 2702 | CG2 | VAL | B | 172 | 7.636 | 21.863 | 69.797 | 1.00 | 15.04 | B |
| ATOM | 2703 | C | VAL | B | 172 | 4.958 | 19.105 | 70.043 | 1.00 | 14.71 | B |
| ATOM | 2704 | O | VAL | B | 172 | 5.033 | 18.013 | 69.483 | 1.00 | 16.77 | B |
| ATOM | 2705 | N | LEU | B | 173 | 4.253 | 19.303 | 71.152 | 1.00 | 16.14 | B |
| ATOM | 2706 | CA | LEU | B | 173 | 3.504 | 18.223 | 71.787 | 1.00 | 18.99 | B |
| ATOM | 2707 | CB | LEU | B | 173 | 2.772 | 18.745 | 73.024 | 1.00 | 18.44 | B |
| ATOM | 2708 | CG | LEU | B | 173 | 3.626 | 19.180 | 74.215 | 1.00 | 19.30 | B |
| ATOM | 2709 | CD1 | LEU | B | 173 | 2.766 | 19.938 | 75.209 | 1.00 | 21.31 | B |
| ATOM | 2710 | CD2 | LEU | B | 173 | 4.261 | 17.957 | 74.867 | 1.00 | 18.38 | B |
| ATOM | 2711 | C | LEU | B | 173 | 2.494 | 17.630 | 70.810 | 1.00 | 21.05 | B |
| ATOM | 2712 | O | LEU | B | 173 | 2.305 | 16.414 | 70.762 | 1.00 | 22.42 | B |
| ATOM | 2713 | N | ALA | B | 174 | 1.847 | 18.494 | 70.032 | 1.00 | 22.14 | B |
| ATOM | 2714 | CA | ALA | B | 174 | 0.852 | 18.054 | 69.058 | 1.00 | 24.87 | B |
| ATOM | 2715 | CB | ALA | B | 174 | 0.286 | 19.252 | 68.305 | 1.00 | 24.14 | B |
| ATOM | 2716 | C | ALA | B | 174 | 1.466 | 17.066 | 68.077 | 1.00 | 26.30 | B |
| ATOM | 2717 | O | ALA | B | 174 | 0.772 | 16.220 | 67.515 | 1.00 | 27.70 | B |
| ATOM | 2718 | N | THR | B | 175 | 2.772 | 17.182 | 67.869 | 1.00 | 28.35 | B |
| ATOM | 2719 | CA | THR | B | 175 | 3.478 | 16.284 | 66.968 | 1.00 | 30.98 | B |
| ATOM | 2720 | CB | THR | B | 175 | 4.301 | 17.062 | 65.928 | 1.00 | 31.51 | B |
| ATOM | 2721 | OG1 | THR | B | 175 | 3.476 | 18.054 | 65.305 | 1.00 | 33.07 | B |
| ATOM | 2722 | CG2 | THR | B | 175 | 4.827 | 16.109 | 64.858 | 1.00 | 32.24 | B |
| ATOM | 2723 | C | THR | B | 175 | 4.416 | 15.398 | 67.783 | 1.00 | 32.12 | B |
| ATOM | 2724 | O | THR | B | 175 | 5.647 | 15.608 | 67.708 | 1.00 | 33.24 | B |
| ATOM | 2725 | OXT | THR | B | 175 | 3.905 | 14.513 | 68.504 | 1.00 | 34.63 | B |
| TER | | | | | | | | | | | |
| ATOM | 2726 | CB | SER | C | 6 | 32.523 | 80.234 | 53.759 | 1.00 | 31.46 | C |
| ATOM | 2727 | OG | SER | C | 6 | 33.236 | 81.268 | 53.102 | 1.00 | 34.48 | C |
| ATOM | 2728 | C | SER | C | 6 | 30.962 | 78.304 | 53.496 | 1.00 | 28.43 | C |
| ATOM | 2729 | O | SER | C | 6 | 31.339 | 77.968 | 54.618 | 1.00 | 27.45 | C |
| ATOM | 2730 | N | SER | C | 6 | 30.556 | 80.422 | 52.259 | 1.00 | 31.79 | C |
| ATOM | 2731 | CA | SER | C | 6 | 31.598 | 79.495 | 52.787 | 1.00 | 30.27 | C |
| ATOM | 2732 | N | LEU | C | 7 | 29.993 | 77.679 | 52.833 | 1.00 | 26.33 | C |
| ATOM | 2733 | CA | LEU | C | 7 | 29.298 | 76.515 | 53.377 | 1.00 | 23.33 | C |
| ATOM | 2734 | CB | LEU | C | 7 | 27.783 | 76.701 | 53.256 | 1.00 | 24.50 | C |
| ATOM | 2735 | CG | LEU | C | 7 | 26.893 | 75.586 | 53.813 | 1.00 | 23.74 | C |
| ATOM | 2736 | CD1 | LEU | C | 7 | 27.197 | 75.366 | 55.286 | 1.00 | 23.30 | C |
| ATOM | 2737 | CD2 | LEU | C | 7 | 25.428 | 75.966 | 53.623 | 1.00 | 25.35 | C |
| ATOM | 2738 | C | LEU | C | 7 | 29.737 | 75.286 | 52.592 | 1.00 | 21.63 | C |
| ATOM | 2739 | O | LEU | C | 7 | 29.413 | 75.142 | 51.411 | 1.00 | 20.47 | C |
| ATOM | 2740 | N | PHE | C | 8 | 30.471 | 74.399 | 53.255 | 1.00 | 18.16 | C |
| ATOM | 2741 | CA | PHE | C | 8 | 30.989 | 73.192 | 52.619 | 1.00 | 18.49 | C |
| ATOM | 2742 | CB | PHE | C | 8 | 32.458 | 73.001 | 53.005 | 1.00 | 20.75 | C |
| ATOM | 2743 | CG | PHE | C | 8 | 33.361 | 74.102 | 52.525 | 1.00 | 26.13 | C |
| ATOM | 2744 | CD1 | PHE | C | 8 | 33.080 | 75.434 | 52.820 | 1.00 | 27.43 | C |
| ATOM | 2745 | CD2 | PHE | C | 8 | 34.488 | 73.810 | 51.770 | 1.00 | 27.61 | C |
| ATOM | 2746 | CE1 | PHE | C | 8 | 33.910 | 76.460 | 52.365 | 1.00 | 29.63 | C |
| ATOM | 2747 | CE2 | PHE | C | 8 | 35.325 | 74.828 | 51.310 | 1.00 | 28.84 | C |
| ATOM | 2748 | CZ | PHE | C | 8 | 35.034 | 76.155 | 51.607 | 1.00 | 28.95 | C |
| ATOM | 2749 | C | PHE | C | 8 | 30.207 | 71.945 | 53.002 | 1.00 | 15.83 | C |
| ATOM | 2750 | O | PHE | C | 8 | 30.271 | 71.500 | 54.142 | 1.00 | 15.57 | C |
| ATOM | 2751 | N | LYS | C | 9 | 29.482 | 71.374 | 52.043 | 1.00 | 14.34 | C |
| ATOM | 2752 | CA | LYS | C | 9 | 28.699 | 70.171 | 52.308 | 1.00 | 12.90 | C |
| ATOM | 2753 | CB | LYS | C | 9 | 27.506 | 70.090 | 51.358 | 1.00 | 12.56 | C |
| ATOM | 2754 | CG | LYS | C | 9 | 26.656 | 68.840 | 51.547 | 1.00 | 12.77 | C |
| ATOM | 2755 | CD | LYS | C | 9 | 25.418 | 68.864 | 50.675 | 1.00 | 13.46 | C |
| ATOM | 2756 | CE | LYS | C | 9 | 24.609 | 67.586 | 50.848 | 1.00 | 13.32 | C |
| ATOM | 2757 | NZ | LYS | C | 9 | 23.403 | 67.563 | 49.973 | 1.00 | 13.77 | C |
| ATOM | 2758 | C | LYS | C | 9 | 29.549 | 68.910 | 52.173 | 1.00 | 11.31 | C |
| ATOM | 2759 | O | LYS | C | 9 | 30.096 | 68.633 | 51.110 | 1.00 | 11.77 | C |
| ATOM | 2760 | N | VAL | C | 10 | 29.661 | 68.157 | 53.263 | 1.00 | 9.62 | C |
| ATOM | 2761 | CA | VAL | C | 10 | 30.437 | 66.923 | 53.284 | 1.00 | 9.56 | C |
| ATOM | 2762 | CB | VAL | C | 10 | 31.550 | 66.986 | 54.356 | 1.00 | 11.04 | C |
| ATOM | 2763 | CG1 | VAL | C | 10 | 32.375 | 65.708 | 54.338 | 1.00 | 12.82 | C |
| ATOM | 2764 | CG2 | VAL | C | 10 | 32.447 | 68.193 | 54.099 | 1.00 | 13.55 | C |
| ATOM | 2765 | C | VAL | C | 10 | 29.470 | 65.791 | 53.619 | 1.00 | 9.21 | C |
| ATOM | 2766 | O | VAL | C | 10 | 28.744 | 65.866 | 54.610 | 1.00 | 10.05 | C |
| ATOM | 2767 | N | ILE | C | 11 | 29.458 | 64.747 | 52.799 | 1.00 | 8.80 | C |
| ATOM | 2768 | CA | ILE | C | 11 | 28.545 | 63.635 | 53.023 | 1.00 | 8.66 | C |
| ATOM | 2769 | CB | ILE | C | 11 | 27.636 | 63.414 | 51.782 | 1.00 | 10.42 | C |
| ATOM | 2770 | CG2 | ILE | C | 11 | 28.480 | 63.079 | 50.561 | 1.00 | 8.26 | C |
| ATOM | 2771 | CG1 | ILE | C | 11 | 26.616 | 62.311 | 52.070 | 1.00 | 12.21 | C |
| ATOM | 2772 | CD1 | ILE | C | 11 | 25.513 | 62.208 | 51.019 | 1.00 | 13.38 | C |
| ATOM | 2773 | C | ILE | C | 11 | 29.256 | 62.334 | 53.392 | 1.00 | 9.56 | C |
| ATOM | 2774 | O | ILE | C | 11 | 30.269 | 61.966 | 52.798 | 1.00 | 10.57 | C |
| ATOM | 2775 | N | LEU | C | 12 | 28.721 | 61.663 | 54.406 | 1.00 | 7.95 | C |
| ATOM | 2776 | CA | LEU | C | 12 | 29.266 | 60.398 | 54.886 | 1.00 | 8.49 | C |
| ATOM | 2777 | CB | LEU | C | 12 | 29.097 | 60.301 | 56.404 | 1.00 | 8.77 | C |
| ATOM | 2778 | CG | LEU | C | 12 | 30.052 | 61.163 | 57.236 | 1.00 | 11.66 | C |
| ATOM | 2779 | CD1 | LEU | C | 12 | 29.459 | 61.463 | 58.594 | 1.00 | 11.58 | C |
| ATOM | 2780 | CD2 | LEU | C | 12 | 31.382 | 60.439 | 57.360 | 1.00 | 13.94 | C |
| ATOM | 2781 | C | LEU | C | 12 | 28.550 | 59.232 | 54.225 | 1.00 | 8.40 | C |
| ATOM | 2782 | O | LEU | C | 12 | 27.329 | 59.115 | 54.310 | 1.00 | 9.12 | C |
| ATOM | 2783 | N | LEU | C | 13 | 29.313 | 58.379 | 53.552 | 1.00 | 9.40 | C |
| ATOM | 2784 | CA | LEU | C | 13 | 28.749 | 57.210 | 52.892 | 1.00 | 9.79 | C |
| ATOM | 2785 | CB | LEU | C | 13 | 28.865 | 57.340 | 51.371 | 1.00 | 9.61 | C |
| ATOM | 2786 | CG | LEU | C | 13 | 28.078 | 58.480 | 50.722 | 1.00 | 11.67 | C |
| ATOM | 2787 | CD1 | LEU | C | 13 | 28.191 | 58.369 | 49.205 | 1.00 | 12.59 | C |
| ATOM | 2788 | CD2 | LEU | C | 13 | 26.620 | 58.416 | 51.152 | 1.00 | 13.78 | C |
| ATOM | 2789 | C | LEU | C | 13 | 29.499 | 55.971 | 53.356 | 1.00 | 9.59 | C |
| ATOM | 2790 | O | LEU | C | 13 | 30.662 | 56.053 | 53.746 | 1.00 | 11.02 | C |
| ATOM | 2791 | N | GLY | C | 14 | 28.832 | 54.825 | 53.322 | 1.00 | 8.86 | C |
| ATOM | 2792 | CA | GLY | C | 14 | 29.483 | 53.597 | 53.746 | 1.00 | 7.08 | C |
| ATOM | 2793 | C | GLY | C | 14 | 28.476 | 52.589 | 54.252 | 1.00 | 7.40 | C |
| ATOM | 2794 | O | GLY | C | 14 | 27.345 | 52.951 | 54.593 | 1.00 | 8.76 | C |
| ATOM | 2795 | N | ASP | C | 15 | 28.891 | 51.326 | 54.306 | 1.00 | 8.49 | C |
| ATOM | 2796 | CA | ASP | C | 15 | 28.030 | 50.239 | 54.759 | 1.00 | 8.48 | C |
| ATOM | 2797 | CB | ASP | C | 15 | 28.808 | 48.919 | 54.802 | 1.00 | 8.78 | C |
| ATOM | 2798 | CG | ASP | C | 15 | 29.030 | 48.319 | 53.430 | 1.00 | 12.16 | C |
| ATOM | 2799 | OD1 | ASP | C | 15 | 28.679 | 48.968 | 52.423 | 1.00 | 12.95 | C |
| ATOM | 2800 | OD2 | ASP | C | 15 | 29.563 | 47.189 | 53.364 | 1.00 | 11.10 | C |
| ATOM | 2801 | C | ASP | C | 15 | 27.422 | 50.483 | 56.128 | 1.00 | 8.22 | C |
| ATOM | 2802 | O | ASP | C | 15 | 27.978 | 51.197 | 56.959 | 1.00 | 6.97 | C |
| ATOM | 2803 | N | GLY | C | 16 | 26.271 | 49.871 | 56.367 | 1.00 | 7.87 | C |
| ATOM | 2804 | CA | GLY | C | 16 | 25.647 | 50.031 | 57.663 | 1.00 | 10.36 | C |
| ATOM | 2805 | C | GLY | C | 16 | 26.588 | 49.494 | 58.725 | 1.00 | 11.09 | C |
| ATOM | 2806 | O | GLY | C | 16 | 27.248 | 48.471 | 58.515 | 1.00 | 12.60 | C |
| ATOM | 2807 | N | GLY | C | 17 | 26.679 | 50.197 | 59.850 | 1.00 | 10.45 | C |
| ATOM | 2808 | CA | GLY | C | 17 | 27.519 | 49.736 | 60.941 | 1.00 | 9.79 | C |
| ATOM | 2809 | C | GLY | C | 17 | 29.002 | 50.060 | 60.942 | 1.00 | 9.57 | C |
| ATOM | 2810 | O | GLY | C | 17 | 29.693 | 49.698 | 61.897 | 1.00 | 9.88 | C |
| ATOM | 2811 | N | VAL | C | 18 | 29.509 | 50.725 | 59.906 | 1.00 | 8.43 | C |
| ATOM | 2812 | CA | VAL | C | 18 | 30.932 | 51.050 | 59.878 | 1.00 | 6.91 | C |
| ATOM | 2813 | CB | VAL | C | 18 | 31.414 | 51.460 | 58.457 | 1.00 | 6.37 | C |
| ATOM | 2814 | CG1 | VAL | C | 18 | 31.256 | 50.280 | 57.495 | 1.00 | 8.92 | C |
| ATOM | 2815 | CG2 | VAL | C | 18 | 30.646 | 52.671 | 57.961 | 1.00 | 8.74 | C |
| ATOM | 2816 | C | VAL | C | 18 | 31.309 | 52.152 | 60.876 | 1.00 | 7.17 | C |
| ATOM | 2817 | O | VAL | C | 18 | 32.480 | 52.308 | 61.222 | 1.00 | 7.39 | C |
| ATOM | 2818 | N | GLY | C | 19 | 30.320 | 52.916 | 61.337 | 1.00 | 7.17 | C |
| ATOM | 2819 | CA | GLY | C | 19 | 30.591 | 53.960 | 62.311 | 1.00 | 6.24 | C |
| ATOM | 2820 | C | GLY | C | 19 | 30.410 | 55.401 | 61.859 | 1.00 | 7.66 | C |
| ATOM | 2821 | O | GLY | C | 19 | 30.941 | 56.316 | 62.488 | 1.00 | 8.73 | C |
| ATOM | 2822 | N | LYS | C | 20 | 29.659 | 55.617 | 60.786 | 1.00 | 7.64 | C |
| ATOM | 2823 | CA | LYS | C | 20 | 29.445 | 56.972 | 60.273 | 1.00 | 7.16 | C |
| ATOM | 2824 | CB | LYS | C | 20 | 28.560 | 56.931 | 59.027 | 1.00 | 5.55 | C |
| ATOM | 2825 | CG | LYS | C | 20 | 29.157 | 56.133 | 57.877 | 1.00 | 6.70 | C |
| ATOM | 2826 | CD | LYS | C | 20 | 28.242 | 56.132 | 56.656 | 1.00 | 7.43 | C |
| ATOM | 2827 | CE | LYS | C | 20 | 26.932 | 55.395 | 56.921 | 1.00 | 9.37 | C |
| ATOM | 2828 | NZ | LYS | C | 20 | 27.153 | 53.992 | 57.385 | 1.00 | 10.86 | C |
| ATOM | 2829 | C | LYS | C | 20 | 28.831 | 57.931 | 61.291 | 1.00 | 8.46 | C |
| ATOM | 2830 | O | LYS | C | 20 | 29.332 | 59.045 | 61.482 | 1.00 | 7.69 | C |
| ATOM | 2831 | N | SER | C | 21 | 27.749 | 57.510 | 61.940 | 1.00 | 8.97 | C |
| ATOM | 2832 | CA | SER | C | 21 | 27.091 | 58.363 | 62.929 | 1.00 | 10.30 | C |
| ATOM | 2833 | CB | SER | C | 21 | 25.789 | 57.718 | 63.414 | 1.00 | 13.28 | C |
| ATOM | 2834 | OG | SER | C | 21 | 24.870 | 57.583 | 62.346 | 1.00 | 20.22 | C |
| ATOM | 2835 | C | SER | C | 21 | 28.002 | 58.631 | 64.117 | 1.00 | 9.74 | C |
| ATOM | 2836 | O | SER | C | 21 | 28.056 | 59.757 | 64.622 | 1.00 | 10.45 | C |
| ATOM | 2837 | N | SER | C | 22 | 28.721 | 57.602 | 64.559 | 1.00 | 8.10 | C |
| ATOM | 2838 | CA | SER | C | 22 | 29.632 | 57.742 | 65.692 | 1.00 | 7.65 | C |
| ATOM | 2839 | CB | SER | C | 22 | 30.229 | 56.380 | 66.076 | 1.00 | 9.61 | C |
| ATOM | 2840 | OG | SER | C | 22 | 29.224 | 55.490 | 66.540 | 1.00 | 10.04 | C |
| ATOM | 2841 | C | SER | C | 22 | 30.754 | 58.728 | 65.379 | 1.00 | 8.26 | C |
| ATOM | 2842 | O | SER | C | 22 | 31.152 | 59.520 | 66.230 | 1.00 | 7.14 | C |
| ATOM | 2843 | N | LEU | C | 23 | 31.262 | 58.674 | 64.153 | 1.00 | 7.74 | C |
| ATOM | 2844 | CA | LEU | C | 23 | 32.323 | 59.574 | 63.730 | 1.00 | 7.46 | C |
| ATOM | 2845 | CB | LEU | C | 23 | 32.843 | 59.159 | 62.351 | 1.00 | 5.50 | C |
| ATOM | 2846 | CG | LEU | C | 23 | 33.741 | 57.922 | 62.334 | 1.00 | 5.06 | C |
| ATOM | 2847 | CD1 | LEU | C | 23 | 33.805 | 57.355 | 60.927 | 1.00 | 5.37 | C |
| ATOM | 2848 | CD2 | LEU | C | 23 | 35.124 | 58.291 | 62.846 | 1.00 | 4.80 | C |
| ATOM | 2849 | C | LEU | C | 23 | 31.817 | 61.015 | 63.689 | 1.00 | 7.93 | C |
| ATOM | 2850 | O | LEU | C | 23 | 32.509 | 61.932 | 64.123 | 1.00 | 9.49 | C |
| ATOM | 2851 | N | MET | C | 24 | 30.611 | 61.214 | 63.166 | 1.00 | 8.25 | C |
| ATOM | 2852 | CA | MET | C | 24 | 30.047 | 62.561 | 63.102 | 1.00 | 10.06 | C |
| ATOM | 2853 | CB | MET | C | 24 | 28.710 | 62.567 | 62.352 | 1.00 | 10.66 | C |
| ATOM | 2854 | CG | MET | C | 24 | 27.995 | 63.922 | 62.398 | 1.00 | 14.08 | C |
| ATOM | 2855 | SD | MET | C | 24 | 26.458 | 63.998 | 61.442 | 1.00 | 18.03 | C |
| ATOM | 2856 | CE | MET | C | 24 | 25.366 | 63.027 | 62.497 | 1.00 | 16.19 | C |
| ATOM | 2857 | C | MET | C | 24 | 29.850 | 63.117 | 64.506 | 1.00 | 10.04 | C |
| ATOM | 2858 | O | MET | C | 24 | 30.203 | 64.263 | 64.782 | 1.00 | 9.75 | C |
| ATOM | 2859 | N | ASN | C | 25 | 29.282 | 62.302 | 65.390 | 1.00 | 10.17 | C |
| ATOM | 2860 | CA | ASN | C | 25 | 29.045 | 62.717 | 66.771 | 1.00 | 11.22 | C |
| ATOM | 2861 | CB | ASN | C | 25 | 28.299 | 61.617 | 67.528 | 1.00 | 12.39 | C |
| ATOM | 2862 | CG | ASN | C | 25 | 26.796 | 61.697 | 67.345 | 1.00 | 15.49 | C |
| ATOM | 2863 | OD1 | ASN | C | 25 | 26.089 | 62.274 | 68.176 | 1.00 | 17.75 | C |
| ATOM | 2864 | ND2 | ASN | C | 25 | 26.299 | 61.129 | 66.251 | 1.00 | 14.01 | C |
| ATOM | 2865 | C | ASN | C | 25 | 30.345 | 63.041 | 67.494 | 1.00 | 12.58 | C |
| ATOM | 2866 | O | ASN | C | 25 | 30.429 | 64.025 | 68.229 | 1.00 | 12.71 | C |
| ATOM | 2867 | N | ARG | C | 26 | 31.356 | 62.203 | 67.288 | 1.00 | 13.19 | C |
| ATOM | 2868 | CA | ARG | C | 26 | 32.653 | 62.401 | 67.924 | 1.00 | 12.65 | C |
| ATOM | 2869 | CB | ARG | C | 26 | 33.575 | 61.225 | 67.591 | 1.00 | 13.72 | C |
| ATOM | 2870 | CG | ARG | C | 26 | 34.933 | 61.258 | 68.265 | 1.00 | 18.09 | C |
| ATOM | 2871 | CD | ARG | C | 26 | 34.796 | 61.406 | 69.772 | 1.00 | 19.71 | C |
| ATOM | 2872 | NE | ARG | C | 26 | 35.726 | 60.546 | 70.495 | 1.00 | 21.60 | C |
| ATOM | 2873 | CZ | ARG | C | 26 | 36.132 | 60.770 | 71.739 | 1.00 | 23.09 | C |
| ATOM | 2874 | NH1 | ARG | C | 26 | 35.689 | 61.833 | 72.397 | 1.00 | 24.21 | C |
| ATOM | 2875 | NH2 | ARG | C | 26 | 36.979 | 59.933 | 72.323 | 1.00 | 22.36 | C |
| ATOM | 2876 | C | ARG | C | 26 | 33.285 | 63.718 | 67.470 | 1.00 | 12.95 | C |
| ATOM | 2877 | O | ARG | C | 26 | 33.865 | 64.451 | 68.273 | 1.00 | 11.51 | C |
| ATOM | 2878 | N | TYR | C | 27 | 33.169 | 64.024 | 66.184 | 1.00 | 12.25 | C |
| ATOM | 2879 | CA | TYR | C | 27 | 33.742 | 65.258 | 65.670 | 1.00 | 13.84 | C |
| ATOM | 2880 | CB | TYR | C | 27 | 33.754 | 65.240 | 64.140 | 1.00 | 12.33 | C |
| ATOM | 2881 | CG | TYR | C | 27 | 34.303 | 66.504 | 63.513 | 1.00 | 12.55 | C |
| ATOM | 2882 | CD1 | TYR | C | 27 | 35.562 | 66.993 | 63.866 | 1.00 | 13.80 | C |
| ATOM | 2883 | CE1 | TYR | C | 27 | 36.071 | 68.162 | 63.295 | 1.00 | 13.90 | C |
| ATOM | 2884 | CD2 | TYR | C | 27 | 33.562 | 67.213 | 62.567 | 1.00 | 12.14 | C |
| ATOM | 2885 | CE2 | TYR | C | 27 | 34.062 | 68.382 | 61.986 | 1.00 | 12.13 | C |
| ATOM | 2886 | CZ | TYR | C | 27 | 35.314 | 68.849 | 62.356 | 1.00 | 14.26 | C |
| ATOM | 2887 | OH | TYR | C | 27 | 35.809 | 70.001 | 61.790 | 1.00 | 15.22 | C |
| ATOM | 2888 | C | TYR | C | 27 | 32.999 | 66.498 | 66.165 | 1.00 | 14.09 | C |
| ATOM | 2889 | O | TYR | C | 27 | 33.619 | 67.484 | 66.551 | 1.00 | 15.29 | C |
| ATOM | 2890 | N | VAL | C | 28 | 31.672 | 66.442 | 66.176 | 1.00 | 16.42 | C |
| ATOM | 2891 | CA | VAL | C | 28 | 30.867 | 67.588 | 66.597 | 1.00 | 18.01 | C |
| ATOM | 2892 | CB | VAL | C | 28 | 29.455 | 67.516 | 65.967 | 1.00 | 18.06 | C |
| ATOM | 2893 | CG1 | VAL | C | 28 | 28.612 | 68.697 | 66.429 | 1.00 | 17.87 | C |
| ATOM | 2894 | CG2 | VAL | C | 28 | 29.570 | 67.512 | 64.446 | 1.00 | 19.43 | C |
| ATOM | 2895 | C | VAL | C | 28 | 30.715 | 67.824 | 68.104 | 1.00 | 19.27 | C |
| ATOM | 2896 | O | VAL | C | 28 | 30.860 | 68.953 | 68.571 | 1.00 | 20.83 | C |
| ATOM | 2897 | N | THR | C | 29 | 30.426 | 66.774 | 68.865 | 1.00 | 20.73 | C |
| ATOM | 2898 | CA | THR | C | 29 | 30.237 | 66.916 | 70.308 | 1.00 | 22.56 | C |
| ATOM | 2899 | CB | THR | C | 29 | 28.972 | 66.179 | 70.781 | 1.00 | 23.50 | C |
| ATOM | 2900 | OG1 | THR | C | 29 | 29.184 | 64.764 | 70.686 | 1.00 | 24.28 | C |
| ATOM | 2901 | CG2 | THR | C | 29 | 27.776 | 66.567 | 69.924 | 1.00 | 23.34 | C |
| ATOM | 2902 | C | THR | C | 29 | 31.397 | 66.367 | 71.121 | 1.00 | 23.41 | C |
| ATOM | 2903 | O | THR | C | 29 | 31.468 | 66.578 | 72.332 | 1.00 | 22.81 | C |
| ATOM | 2904 | N | ASN | C | 30 | 32.297 | 65.655 | 70.453 | 1.00 | 25.11 | C |
| ATOM | 2905 | CA | ASN | C | 30 | 33.443 | 65.051 | 71.112 | 1.00 | 26.08 | C |
| ATOM | 2906 | CB | ASN | C | 30 | 34.274 | 66.110 | 71.840 | 1.00 | 27.75 | C |
| ATOM | 2907 | CG | ASN | C | 30 | 35.613 | 65.573 | 72.294 | 1.00 | 28.65 | C |
| ATOM | 2908 | OD1 | ASN | C | 30 | 36.285 | 64.857 | 71.550 | 1.00 | 29.31 | C |
| ATOM | 2909 | ND2 | ASN | C | 30 | 36.014 | 65.919 | 73.511 | 1.00 | 29.77 | C |
| ATOM | 2910 | C | ASN | C | 30 | 32.956 | 63.996 | 72.097 | 1.00 | 26.56 | C |
| ATOM | 2911 | O | ASN | C | 30 | 33.492 | 63.848 | 73.195 | 1.00 | 26.71 | C |
| ATOM | 2912 | N | LYS | C | 31 | 31.926 | 63.266 | 71.685 | 1.00 | 27.47 | C |
| ATOM | 2913 | CA | LYS | C | 31 | 31.337 | 62.213 | 72.500 | 1.00 | 28.31 | C |
| ATOM | 2914 | CB | LYS | C | 31 | 29.953 | 62.642 | 72.987 | 1.00 | 29.28 | C |
| ATOM | 2915 | CG | LYS | C | 31 | 29.920 | 64.002 | 73.663 | 1.00 | 32.61 | C |
| ATOM | 2916 | CD | LYS | C | 31 | 28.490 | 64.499 | 73.825 | 1.00 | 34.33 | C |
| ATOM | 2917 | CE | LYS | C | 31 | 28.455 | 65.913 | 74.394 | 1.00 | 36.33 | C |
| ATOM | 2918 | NZ | LYS | C | 31 | 27.077 | 66.483 | 74.398 | 1.00 | 34.85 | C |
| ATOM | 2919 | C | LYS | C | 31 | 31.201 | 60.968 | 71.633 | 1.00 | 28.01 | C |
| ATOM | 2920 | O | LYS | C | 31 | 30.798 | 61.057 | 70.473 | 1.00 | 26.02 | C |
| ATOM | 2921 | N | PHE | C | 32 | 31.544 | 59.810 | 72.186 | 1.00 | 28.05 | C |
| ATOM | 2922 | CA | PHE | C | 32 | 31.429 | 58.577 | 71.425 | 1.00 | 28.71 | C |
| ATOM | 2923 | CB | PHE | C | 32 | 32.509 | 57.576 | 71.842 | 1.00 | 27.74 | C |
| ATOM | 2924 | CG | PHE | C | 32 | 32.375 | 56.234 | 71.178 | 1.00 | 26.38 | C |
| ATOM | 2925 | CD1 | PHE | C | 32 | 32.160 | 56.140 | 69.805 | 1.00 | 24.47 | C |
| ATOM | 2926 | CD2 | PHE | C | 32 | 32.457 | 55.064 | 71.926 | 1.00 | 25.67 | C |
| ATOM | 2927 | CE1 | PHE | C | 32 | 32.026 | 54.905 | 69.189 | 1.00 | 23.59 | C |
| ATOM | 2928 | CE2 | PHE | C | 32 | 32.325 | 53.820 | 71.317 | 1.00 | 25.14 | C |
| ATOM | 2929 | CZ | PHE | C | 32 | 32.108 | 53.741 | 69.945 | 1.00 | 24.00 | C |
| ATOM | 2930 | C | PHE | C | 32 | 30.046 | 57.957 | 71.595 | 1.00 | 30.02 | C |
| ATOM | 2931 | O | PHE | C | 32 | 29.834 | 57.089 | 72.439 | 1.00 | 30.17 | C |
| ATOM | 2932 | N | ASP | C | 33 | 29.107 | 58.429 | 70.786 | 1.00 | 30.97 | C |
| ATOM | 2933 | CA | ASP | C | 33 | 27.740 | 57.931 | 70.806 | 1.00 | 33.82 | C |
| ATOM | 2934 | CB | ASP | C | 33 | 27.022 | 58.352 | 72.095 | 1.00 | 36.18 | C |
| ATOM | 2935 | CG | ASP | C | 33 | 26.936 | 59.858 | 72.256 | 1.00 | 38.61 | C |
| ATOM | 2936 | OD1 | ASP | C | 33 | 26.457 | 60.533 | 71.320 | 1.00 | 38.06 | C |
| ATOM | 2937 | OD2 | ASP | C | 33 | 27.338 | 60.365 | 73.327 | 1.00 | 40.50 | C |
| ATOM | 2938 | C | ASP | C | 33 | 26.996 | 58.472 | 69.592 | 1.00 | 32.56 | C |
| ATOM | 2939 | O | ASP | C | 33 | 27.589 | 59.113 | 68.727 | 1.00 | 33.19 | C |
| ATOM | 2940 | N | THR | C | 34 | 25.698 | 58.205 | 69.526 | 1.00 | 30.85 | C |
| ATOM | 2941 | CA | THR | C | 34 | 24.880 | 58.667 | 68.414 | 1.00 | 28.98 | C |
| ATOM | 2942 | CB | THR | C | 34 | 24.408 | 57.477 | 67.555 | 1.00 | 30.25 | C |
| ATOM | 2943 | OG1 | THR | C | 34 | 23.700 | 56.540 | 68.379 | 1.00 | 28.13 | C |
| ATOM | 2944 | CG2 | THR | C | 34 | 25.605 | 56.775 | 66.923 | 1.00 | 29.37 | C |
| ATOM | 2945 | C | THR | C | 34 | 23.672 | 59.410 | 68.972 | 1.00 | 28.15 | C |
| ATOM | 2946 | O | THR | C | 34 | 22.545 | 59.224 | 68.516 | 1.00 | 25.81 | C |
| ATOM | 2947 | N | GLN | C | 35 | 23.933 | 60.264 | 69.959 | 1.00 | 26.70 | C |
| ATOM | 2948 | CA | GLN | C | 35 | 22.891 | 61.034 | 70.628 | 1.00 | 25.89 | C |
| ATOM | 2949 | CB | GLN | C | 35 | 23.359 | 61.419 | 72.035 | 1.00 | 26.74 | C |
| ATOM | 2950 | CG | GLN | C | 35 | 23.399 | 60.249 | 73.003 | 1.00 | 25.94 | C |
| ATOM | 2951 | CD | GLN | C | 35 | 22.035 | 59.610 | 73.182 | 1.00 | 27.49 | C |
| ATOM | 2952 | OE1 | GLN | C | 35 | 21.073 | 60.273 | 73.575 | 1.00 | 28.14 | C |
| ATOM | 2953 | NE2 | GLN | C | 35 | 21.943 | 58.316 | 72.894 | 1.00 | 28.65 | C |
| ATOM | 2954 | C | GLN | C | 35 | 22.383 | 62.279 | 69.906 | 1.00 | 25.35 | C |
| ATOM | 2955 | O | GLN | C | 35 | 21.263 | 62.721 | 70.164 | 1.00 | 25.47 | C |
| ATOM | 2956 | N | LEU | C | 36 | 23.187 | 62.848 | 69.010 | 1.00 | 24.05 | C |
| ATOM | 2957 | CA | LEU | C | 36 | 22.756 | 64.045 | 68.287 | 1.00 | 24.02 | C |
| ATOM | 2958 | CB | LEU | C | 36 | 23.741 | 64.391 | 67.164 | 1.00 | 22.82 | C |
| ATOM | 2959 | CG | LEU | C | 36 | 25.019 | 65.121 | 67.593 | 1.00 | 22.38 | C |
| ATOM | 2960 | CD1 | LEU | C | 36 | 25.964 | 65.247 | 66.409 | 1.00 | 20.92 | C |
| ATOM | 2961 | CD2 | LEU | C | 36 | 24.665 | 66.501 | 68.138 | 1.00 | 20.97 | C |
| ATOM | 2962 | C | LEU | C | 36 | 21.356 | 63.876 | 67.706 | 1.00 | 24.07 | C |
| ATOM | 2963 | O | LEU | C | 36 | 20.506 | 64.762 | 67.838 | 1.00 | 24.00 | C |
| ATOM | 2964 | N | PHE | C | 37 | 21.121 | 62.737 | 67.063 | 1.00 | 23.99 | C |
| ATOM | 2965 | CA | PHE | C | 37 | 19.821 | 62.454 | 66.469 | 1.00 | 24.59 | C |
| ATOM | 2966 | CB | PHE | C | 37 | 19.914 | 62.522 | 64.944 | 1.00 | 23.02 | C |
| ATOM | 2967 | CG | PHE | C | 37 | 20.537 | 63.791 | 64.434 | 1.00 | 20.91 | C |
| ATOM | 2968 | CD1 | PHE | C | 37 | 21.915 | 63.881 | 64.254 | 1.00 | 20.48 | C |
| ATOM | 2969 | CD2 | PHE | C | 37 | 19.749 | 64.905 | 64.157 | 1.00 | 19.14 | C |
| ATOM | 2970 | CE1 | PHE | C | 37 | 22.501 | 65.061 | 63.805 | 1.00 | 18.81 | C |
| ATOM | 2971 | CE2 | PHE | C | 37 | 20.325 | 66.094 | 63.706 | 1.00 | 19.17 | C |
| ATOM | 2972 | CZ | PHE | C | 37 | 21.704 | 66.173 | 63.529 | 1.00 | 19.75 | C |
| ATOM | 2973 | C | PHE | C | 37 | 19.345 | 61.075 | 66.911 | 1.00 | 25.20 | C |
| ATOM | 2974 | O | PHE | C | 37 | 20.116 | 60.113 | 66.916 | 1.00 | 25.41 | C |
| ATOM | 2975 | N | HIS | C | 38 | 18.070 | 60.982 | 67.275 | 1.00 | 26.20 | C |
| ATOM | 2976 | CA | HIS | C | 38 | 17.507 | 59.724 | 67.751 | 1.00 | 27.27 | C |
| ATOM | 2977 | CB | HIS | C | 38 | 16.589 | 59.996 | 68.945 | 1.00 | 27.79 | C |
| ATOM | 2978 | CG | HIS | C | 38 | 17.252 | 60.753 | 70.054 | 1.00 | 28.55 | C |
| ATOM | 2979 | CD2 | HIS | C | 38 | 16.987 | 61.971 | 70.583 | 1.00 | 28.43 | C |
| ATOM | 2980 | ND1 | HIS | C | 38 | 18.341 | 60.265 | 70.743 | 1.00 | 29.42 | C |
| ATOM | 2981 | CE1 | HIS | C | 38 | 18.720 | 61.149 | 71.648 | 1.00 | 29.15 | C |
| ATOM | 2982 | NE2 | HIS | C | 38 | 17.915 | 62.194 | 71.572 | 1.00 | 29.27 | C |
| ATOM | 2983 | C | HIS | C | 38 | 16.756 | 58.898 | 66.711 | 1.00 | 28.19 | C |
| ATOM | 2984 | O | HIS | C | 38 | 16.158 | 57.879 | 67.051 | 1.00 | 28.34 | C |
| ATOM | 2985 | N | THR | C | 39 | 16.773 | 59.325 | 65.451 | 1.00 | 28.90 | C |
| ATOM | 2986 | CA | THR | C | 39 | 16.083 | 58.569 | 64.407 | 1.00 | 29.47 | C |
| ATOM | 2987 | CB | THR | C | 39 | 14.895 | 59.360 | 63.812 | 1.00 | 28.92 | C |
| ATOM | 2988 | OG1 | THR | C | 39 | 15.356 | 60.619 | 63.303 | 1.00 | 29.56 | C |
| ATOM | 2989 | CG2 | THR | C | 39 | 13.830 | 59.594 | 64.872 | 1.00 | 30.19 | C |
| ATOM | 2990 | C | THR | C | 39 | 17.017 | 58.170 | 63.272 | 1.00 | 28.99 | C |
| ATOM | 2991 | O | THR | C | 39 | 18.052 | 58.804 | 63.052 | 1.00 | 29.98 | C |
| ATOM | 2992 | N | ILE | C | 40 | 16.642 | 57.110 | 62.560 | 1.00 | 28.11 | C |
| ATOM | 2993 | CA | ILE | C | 40 | 17.426 | 56.607 | 61.436 | 1.00 | 26.65 | C |
| ATOM | 2994 | CB | ILE | C | 40 | 17.333 | 55.063 | 61.332 | 1.00 | 28.76 | C |
| ATOM | 2995 | CG2 | ILE | C | 40 | 18.093 | 54.419 | 62.480 | 1.00 | 30.22 | C |
| ATOM | 2996 | CG1 | ILE | C | 40 | 15.866 | 54.612 | 61.344 | 1.00 | 30.20 | C |
| ATOM | 2997 | CD1 | ILE | C | 40 | 15.155 | 54.712 | 60.005 | 1.00 | 29.09 | C |
| ATOM | 2998 | C | ILE | C | 40 | 16.947 | 57.226 | 60.129 | 1.00 | 24.69 | C |
| ATOM | 2999 | O | ILE | C | 40 | 15.757 | 57.498 | 59.954 | 1.00 | 26.32 | C |
| ATOM | 3000 | N | GLY | C | 41 | 17.881 | 57.439 | 59.211 | 1.00 | 20.61 | C |
| ATOM | 3001 | CA | GLY | C | 41 | 17.549 | 58.042 | 57.934 | 1.00 | 14.46 | C |
| ATOM | 3002 | C | GLY | C | 41 | 18.696 | 58.939 | 57.518 | 1.00 | 12.02 | C |
| ATOM | 3003 | O | GLY | C | 41 | 19.813 | 58.469 | 57.340 | 1.00 | 11.54 | C |
| ATOM | 3004 | N | VAL | C | 42 | 18.422 | 60.231 | 57.375 | 1.00 | 9.12 | C |
| ATOM | 3005 | CA | VAL | C | 42 | 19.440 | 61.197 | 56.989 | 1.00 | 8.64 | C |
| ATOM | 3006 | CB | VAL | C | 42 | 19.176 | 61.768 | 55.576 | 1.00 | 7.98 | C |
| ATOM | 3007 | CG1 | VAL | C | 42 | 20.160 | 62.896 | 55.278 | 1.00 | 9.92 | C |
| ATOM | 3008 | CG2 | VAL | C | 42 | 19.296 | 60.659 | 54.532 | 1.00 | 9.44 | C |
| ATOM | 3009 | C | VAL | C | 42 | 19.427 | 62.351 | 57.978 | 1.00 | 8.45 | C |
| ATOM | 3010 | O | VAL | C | 42 | 18.365 | 62.864 | 58.327 | 1.00 | 7.03 | C |
| ATOM | 3011 | N | GLU | C | 43 | 20.610 | 62.750 | 58.439 | 1.00 | 8.60 | C |
| ATOM | 3012 | CA | GLU | C | 43 | 20.723 | 63.859 | 59.377 | 1.00 | 9.38 | C |
| ATOM | 3013 | CB | GLU | C | 43 | 21.020 | 63.351 | 60.794 | 1.00 | 10.85 | C |
| ATOM | 3014 | CG | GLU | C | 43 | 20.121 | 62.219 | 61.295 | 1.00 | 10.83 | C |
| ATOM | 3015 | CD | GLU | C | 43 | 20.457 | 60.870 | 60.679 | 1.00 | 12.30 | C |
| ATOM | 3016 | OE1 | GLU | C | 43 | 21.658 | 60.554 | 60.545 | 1.00 | 15.42 | C |
| ATOM | 3017 | OE2 | GLU | C | 43 | 19.523 | 60.113 | 60.347 | 1.00 | 12.69 | C |
| ATOM | 3018 | C | GLU | C | 43 | 21.864 | 64.773 | 58.945 | 1.00 | 9.03 | C |
| ATOM | 3019 | O | GLU | C | 43 | 22.812 | 64.330 | 58.301 | 1.00 | 10.58 | C |
| ATOM | 3020 | N | PHE | C | 44 | 21.770 | 66.053 | 59.285 | 1.00 | 9.50 | C |
| ATOM | 3021 | CA | PHE | C | 44 | 22.842 | 66.980 | 58.955 | 1.00 | 9.38 | C |
| ATOM | 3022 | CB | PHE | C | 44 | 22.758 | 67.461 | 57.494 | 1.00 | 9.27 | C |
| ATOM | 3023 | CG | PHE | C | 44 | 21.524 | 68.266 | 57.167 | 1.00 | 10.12 | C |
| ATOM | 3024 | CD1 | PHE | C | 44 | 20.351 | 67.637 | 56.765 | 1.00 | 9.73 | C |
| ATOM | 3025 | CD2 | PHE | C | 44 | 21.552 | 69.655 | 57.227 | 1.00 | 11.40 | C |
| ATOM | 3026 | CE1 | PHE | C | 44 | 19.221 | 68.383 | 56.421 | 1.00 | 8.28 | C |
| ATOM | 3027 | CE2 | PHE | C | 44 | 20.430 | 70.410 | 56.888 | 1.00 | 12.12 | C |
| ATOM | 3028 | CZ | PHE | C | 44 | 19.263 | 69.772 | 56.482 | 1.00 | 10.74 | C |
| ATOM | 3029 | C | PHE | C | 44 | 22.875 | 68.168 | 59.905 | 1.00 | 10.69 | C |
| ATOM | 3030 | O | PHE | C | 44 | 21.867 | 68.522 | 60.525 | 1.00 | 9.21 | C |
| ATOM | 3031 | N | LEU | C | 45 | 24.052 | 68.767 | 60.029 | 1.00 | 10.80 | C |
| ATOM | 3032 | CA | LEU | C | 45 | 24.230 | 69.907 | 60.910 | 1.00 | 13.10 | C |
| ATOM | 3033 | CB | LEU | C | 45 | 24.405 | 69.417 | 62.350 | 1.00 | 13.29 | C |
| ATOM | 3034 | CG | LEU | C | 45 | 25.574 | 68.470 | 62.641 | 1.00 | 15.64 | C |
| ATOM | 3035 | CD1 | LEU | C | 45 | 26.877 | 69.248 | 62.655 | 1.00 | 17.63 | C |
| ATOM | 3036 | CD2 | LEU | C | 45 | 25.358 | 67.802 | 63.987 | 1.00 | 17.19 | C |
| ATOM | 3037 | C | LEU | C | 45 | 25.434 | 70.731 | 60.477 | 1.00 | 14.30 | C |
| ATOM | 3038 | O | LEU | C | 45 | 26.276 | 70.251 | 59.713 | 1.00 | 14.07 | C |
| ATOM | 3039 | N | ASN | C | 46 | 25.512 | 71.963 | 60.975 | 1.00 | 14.97 | C |
| ATOM | 3040 | CA | ASN | C | 46 | 26.606 | 72.870 | 60.645 | 1.00 | 16.08 | C |
| ATOM | 3041 | CB | ASN | C | 46 | 26.082 | 74.286 | 60.372 | 1.00 | 15.82 | C |
| ATOM | 3042 | CG | ASN | C | 46 | 25.235 | 74.380 | 59.120 | 1.00 | 15.79 | C |
| ATOM | 3043 | OD1 | ASN | C | 46 | 25.319 | 73.538 | 58.227 | 1.00 | 14.92 | C |
| ATOM | 3044 | ND2 | ASN | C | 46 | 24.428 | 75.431 | 59.037 | 1.00 | 17.76 | C |
| ATOM | 3045 | C | ASN | C | 46 | 27.641 | 72.972 | 61.761 | 1.00 | 17.07 | C |
| ATOM | 3046 | O | ASN | C | 46 | 27.298 | 72.943 | 62.943 | 1.00 | 17.01 | C |
| ATOM | 3047 | N | LYS | C | 47 | 28.908 | 73.096 | 61.376 | 1.00 | 16.63 | C |
| ATOM | 3048 | CA | LYS | C | 47 | 29.990 | 73.254 | 62.337 | 1.00 | 17.12 | C |
| ATOM | 3049 | CB | LYS | C | 47 | 30.720 | 71.936 | 62.584 | 1.00 | 17.73 | C |
| ATOM | 3050 | CG | LYS | C | 47 | 31.884 | 72.079 | 63.559 | 1.00 | 17.75 | C |
| ATOM | 3051 | CD | LYS | C | 47 | 32.492 | 70.738 | 63.922 | 1.00 | 20.66 | C |
| ATOM | 3052 | CE | LYS | C | 47 | 33.730 | 70.915 | 64.784 | 1.00 | 20.69 | C |
| ATOM | 3053 | NZ | LYS | C | 47 | 33.447 | 71.716 | 66.010 | 1.00 | 23.44 | C |
| ATOM | 3054 | C | LYS | C | 47 | 30.967 | 74.276 | 61.776 | 1.00 | 17.98 | C |
| ATOM | 3055 | O | LYS | C | 47 | 31.491 | 74.094 | 60.679 | 1.00 | 16.05 | C |
| ATOM | 3056 | N | ASP | C | 48 | 31.200 | 75.355 | 62.519 | 1.00 | 18.86 | C |
| ATOM | 3057 | CA | ASP | C | 48 | 32.117 | 76.394 | 62.064 | 1.00 | 22.36 | C |
| ATOM | 3058 | CB | ASP | C | 48 | 31.748 | 77.751 | 62.670 | 1.00 | 24.65 | C |
| ATOM | 3059 | CG | ASP | C | 48 | 30.339 | 78.181 | 62.324 | 1.00 | 27.43 | C |
| ATOM | 3060 | OD1 | ASP | C | 48 | 29.928 | 77.992 | 61.160 | 1.00 | 29.06 | C |
| ATOM | 3061 | OD2 | ASP | C | 48 | 29.646 | 78.718 | 63.213 | 1.00 | 29.58 | C |
| ATOM | 3062 | C | ASP | C | 48 | 33.554 | 76.059 | 62.431 | 1.00 | 23.62 | C |
| ATOM | 3063 | O | ASP | C | 48 | 33.828 | 75.570 | 63.527 | 1.00 | 22.88 | C |
| ATOM | 3064 | N | LEU | C | 49 | 34.469 | 76.325 | 61.506 | 1.00 | 25.07 | C |
| ATOM | 3065 | CA | LEU | C | 49 | 35.879 | 76.058 | 61.743 | 1.00 | 28.62 | C |
| ATOM | 3066 | CB | LEU | C | 49 | 36.236 | 74.625 | 61.328 | 1.00 | 29.31 | C |
| ATOM | 3067 | CG | LEU | C | 49 | 36.120 | 74.195 | 59.863 | 1.00 | 30.39 | C |
| ATOM | 3068 | CD1 | LEU | C | 49 | 36.764 | 72.827 | 59.695 | 1.00 | 32.22 | C |
| ATOM | 3069 | CD2 | LEU | C | 49 | 34.666 | 74.155 | 59.431 | 1.00 | 30.59 | C |
| ATOM | 3070 | C | LEU | C | 49 | 36.748 | 77.053 | 60.991 | 1.00 | 30.76 | C |
| ATOM | 3071 | O | LEU | C | 49 | 36.325 | 77.635 | 59.992 | 1.00 | 29.72 | C |
| ATOM | 3072 | N | GLU | C | 50 | 37.963 | 77.251 | 61.492 | 1.00 | 33.70 | C |
| ATOM | 3073 | CA | GLU | C | 50 | 38.913 | 78.174 | 60.887 | 1.00 | 36.61 | C |
| ATOM | 3074 | CB | GLU | C | 50 | 39.725 | 78.878 | 61.980 | 1.00 | 38.21 | C |
| ATOM | 3075 | CG | GLU | C | 50 | 40.692 | 79.947 | 61.476 | 1.00 | 41.21 | C |
| ATOM | 3076 | CD | GLU | C | 50 | 39.994 | 81.242 | 61.100 | 1.00 | 42.45 | C |
| ATOM | 3077 | OE1 | GLU | C | 50 | 39.115 | 81.214 | 60.212 | 1.00 | 43.98 | C |
| ATOM | 3078 | OE2 | GLU | C | 50 | 40.326 | 82.291 | 61.694 | 1.00 | 43.66 | C |
| ATOM | 3079 | C | GLU | C | 50 | 39.853 | 77.404 | 59.969 | 1.00 | 37.45 | C |
| ATOM | 3080 | O | GLU | C | 50 | 40.452 | 76.406 | 60.371 | 1.00 | 37.39 | C |
| ATOM | 3081 | N | VAL | C | 51 | 39.971 | 77.863 | 58.730 | 1.00 | 38.02 | C |
| ATOM | 3082 | CA | VAL | C | 51 | 40.851 | 77.219 | 57.769 | 1.00 | 38.42 | C |
| ATOM | 3083 | CB | VAL | C | 51 | 40.071 | 76.276 | 56.835 | 1.00 | 38.53 | C |
| ATOM | 3084 | CG1 | VAL | C | 51 | 41.040 | 75.507 | 55.954 | 1.00 | 38.56 | C |
| ATOM | 3085 | CG2 | VAL | C | 51 | 39.221 | 75.318 | 57.653 | 1.00 | 38.79 | C |
| ATOM | 3086 | C | VAL | C | 51 | 41.547 | 78.288 | 56.935 | 1.00 | 38.56 | C |
| ATOM | 3087 | O | VAL | C | 51 | 40.920 | 78.953 | 56.106 | 1.00 | 37.89 | C |
| ATOM | 3088 | N | ASP | C | 52 | 42.844 | 78.457 | 57.170 | 1.00 | 38.79 | C |
| ATOM | 3089 | CA | ASP | C | 52 | 43.630 | 79.449 | 56.451 | 1.00 | 39.47 | C |
| ATOM | 3090 | CB | ASP | C | 52 | 43.710 | 79.088 | 54.964 | 1.00 | 39.77 | C |
| ATOM | 3091 | CG | ASP | C | 52 | 44.154 | 77.656 | 54.736 | 1.00 | 41.10 | C |
| ATOM | 3092 | OD1 | ASP | C | 52 | 45.202 | 77.260 | 55.286 | 1.00 | 41.75 | C |
| ATOM | 3093 | OD2 | ASP | C | 52 | 43.457 | 76.923 | 54.003 | 1.00 | 42.52 | C |
| ATOM | 3094 | C | ASP | C | 52 | 43.011 | 80.836 | 56.612 | 1.00 | 39.60 | C |
| ATOM | 3095 | O | ASP | C | 52 | 42.683 | 81.500 | 55.629 | 1.00 | 40.43 | C |
| ATOM | 3096 | N | GLY | C | 53 | 42.847 | 81.264 | 57.859 | 1.00 | 39.56 | C |
| ATOM | 3097 | CA | GLY | C | 53 | 42.278 | 82.573 | 58.129 | 1.00 | 39.66 | C |
| ATOM | 3098 | C | GLY | C | 53 | 40.889 | 82.791 | 57.553 | 1.00 | 39.57 | C |
| ATOM | 3099 | O | GLY | C | 53 | 40.353 | 83.898 | 57.621 | 1.00 | 40.04 | C |
| ATOM | 3100 | N | HIS | C | 54 | 40.303 | 81.741 | 56.985 | 1.00 | 38.78 | C |
| ATOM | 3101 | CA | HIS | C | 54 | 38.969 | 81.838 | 56.402 | 1.00 | 37.66 | C |
| ATOM | 3102 | CB | HIS | C | 54 | 38.971 | 81.288 | 54.971 | 1.00 | 39.00 | C |
| ATOM | 3103 | CG | HIS | C | 54 | 39.832 | 82.068 | 54.027 | 1.00 | 40.62 | C |
| ATOM | 3104 | CD2 | HIS | C | 54 | 40.863 | 81.685 | 53.236 | 1.00 | 40.72 | C |
| ATOM | 3105 | ND1 | HIS | C | 54 | 39.675 | 83.422 | 53.823 | 1.00 | 40.94 | C |
| ATOM | 3106 | CE1 | HIS | C | 54 | 40.574 | 83.840 | 52.949 | 1.00 | 41.50 | C |
| ATOM | 3107 | NE2 | HIS | C | 54 | 41.307 | 82.806 | 52.577 | 1.00 | 41.20 | C |
| ATOM | 3108 | C | HIS | C | 54 | 37.927 | 81.094 | 57.230 | 1.00 | 35.31 | C |
| ATOM | 3109 | O | HIS | C | 54 | 38.018 | 79.880 | 57.420 | 1.00 | 34.67 | C |
| ATOM | 3110 | N | PHE | C | 55 | 36.942 | 81.834 | 57.729 | 1.00 | 33.52 | C |
| ATOM | 3111 | CA | PHE | C | 55 | 35.876 | 81.243 | 58.523 | 1.00 | 31.41 | C |
| ATOM | 3112 | CB | PHE | C | 55 | 35.115 | 82.326 | 59.292 | 1.00 | 33.72 | C |
| ATOM | 3113 | CG | PHE | C | 55 | 35.969 | 83.108 | 60.251 | 1.00 | 35.50 | C |
| ATOM | 3114 | CD1 | PHE | C | 55 | 36.605 | 82.474 | 61.315 | 1.00 | 35.64 | C |
| ATOM | 3115 | CD2 | PHE | C | 55 | 36.140 | 84.481 | 60.090 | 1.00 | 36.18 | C |
| ATOM | 3116 | CE1 | PHE | C | 55 | 37.400 | 83.197 | 62.206 | 1.00 | 37.24 | C |
| ATOM | 3117 | CE2 | PHE | C | 55 | 36.934 | 85.214 | 60.975 | 1.00 | 36.98 | C |
| ATOM | 3118 | CZ | PHE | C | 55 | 37.565 | 84.571 | 62.035 | 1.00 | 36.88 | C |
| ATOM | 3119 | C | PHE | C | 55 | 34.916 | 80.522 | 57.585 | 1.00 | 29.49 | C |
| ATOM | 3120 | O | PHE | C | 55 | 34.281 | 81.148 | 56.736 | 1.00 | 28.91 | C |
| ATOM | 3121 | N | VAL | C | 56 | 34.820 | 79.206 | 57.735 | 1.00 | 26.31 | C |
| ATOM | 3122 | CA | VAL | C | 56 | 33.925 | 78.413 | 56.902 | 1.00 | 23.75 | C |
| ATOM | 3123 | CB | VAL | C | 56 | 34.708 | 77.458 | 55.972 | 1.00 | 24.50 | C |
| ATOM | 3124 | CG1 | VAL | C | 56 | 35.680 | 78.250 | 55.109 | 1.00 | 24.89 | C |
| ATOM | 3125 | CG2 | VAL | C | 56 | 35.453 | 76.417 | 56.797 | 1.00 | 25.95 | C |
| ATOM | 3126 | C | VAL | C | 56 | 32.992 | 77.576 | 57.768 | 1.00 | 21.01 | C |
| ATOM | 3127 | O | VAL | C | 56 | 33.282 | 77.307 | 58.932 | 1.00 | 19.97 | C |
| ATOM | 3128 | N | THR | C | 57 | 31.863 | 77.181 | 57.196 | 1.00 | 19.59 | C |
| ATOM | 3129 | CA | THR | C | 57 | 30.909 | 76.351 | 57.909 | 1.00 | 16.35 | C |
| ATOM | 3130 | CB | THR | C | 57 | 29.505 | 76.979 | 57.949 | 1.00 | 16.93 | C |
| ATOM | 3131 | OG1 | THR | C | 57 | 29.556 | 78.219 | 58.662 | 1.00 | 17.42 | C |
| ATOM | 3132 | CG2 | THR | C | 57 | 28.528 | 76.042 | 58.653 | 1.00 | 14.54 | C |
| ATOM | 3133 | C | THR | C | 57 | 30.824 | 75.027 | 57.179 | 1.00 | 16.55 | C |
| ATOM | 3134 | O | THR | C | 57 | 30.510 | 74.978 | 55.988 | 1.00 | 17.08 | C |
| ATOM | 3135 | N | MET | C | 58 | 31.134 | 73.955 | 57.895 | 1.00 | 15.19 | C |
| ATOM | 3136 | CA | MET | C | 58 | 31.078 | 72.625 | 57.322 | 1.00 | 14.48 | C |
| ATOM | 3137 | CB | MET | C | 58 | 32.158 | 71.732 | 57.934 | 1.00 | 16.84 | C |
| ATOM | 3138 | CG | MET | C | 58 | 32.260 | 70.363 | 57.284 | 1.00 | 21.18 | C |
| ATOM | 3139 | SD | MET | C | 58 | 33.200 | 69.191 | 58.280 | 1.00 | 25.81 | C |
| ATOM | 3140 | CE | MET | C | 58 | 34.775 | 70.040 | 58.415 | 1.00 | 25.78 | C |
| ATOM | 3141 | C | MET | C | 58 | 29.713 | 72.050 | 57.656 | 1.00 | 12.64 | C |
| ATOM | 3142 | O | MET | C | 58 | 29.300 | 72.054 | 58.815 | 1.00 | 11.89 | C |
| ATOM | 3143 | N | GLN | C | 59 | 29.007 | 71.572 | 56.640 | 1.00 | 10.32 | C |
| ATOM | 3144 | CA | GLN | C | 59 | 27.701 | 70.980 | 56.867 | 1.00 | 10.37 | C |
| ATOM | 3145 | CB | GLN | C | 59 | 26.661 | 71.576 | 55.921 | 1.00 | 9.30 | C |
| ATOM | 3146 | CG | GLN | C | 59 | 25.240 | 71.080 | 56.180 | 1.00 | 10.21 | C |
| ATOM | 3147 | CD | GLN | C | 59 | 24.204 | 71.905 | 55.451 | 1.00 | 11.49 | C |
| ATOM | 3148 | OE1 | GLN | C | 59 | 23.803 | 72.981 | 55.915 | 1.00 | 14.94 | C |
| ATOM | 3149 | NE2 | GLN | C | 59 | 23.775 | 71.421 | 54.298 | 1.00 | 8.34 | C |
| ATOM | 3150 | C | GLN | C | 59 | 27.869 | 69.490 | 56.625 | 1.00 | 9.47 | C |
| ATOM | 3151 | O | GLN | C | 59 | 28.042 | 69.043 | 55.492 | 1.00 | 9.47 | C |
| ATOM | 3152 | N | ILE | C | 60 | 27.838 | 68.735 | 57.716 | 1.00 | 9.56 | C |
| ATOM | 3153 | CA | ILE | C | 60 | 28.020 | 67.295 | 57.674 | 1.00 | 8.02 | C |
| ATOM | 3154 | CB | ILE | C | 60 | 28.690 | 66.803 | 58.973 | 1.00 | 7.90 | C |
| ATOM | 3155 | CG2 | ILE | C | 60 | 28.913 | 65.300 | 58.912 | 1.00 | 7.20 | C |
| ATOM | 3156 | CG1 | ILE | C | 60 | 30.012 | 67.550 | 59.181 | 1.00 | 9.86 | C |
| ATOM | 3157 | CD1 | ILE | C | 60 | 30.639 | 67.301 | 60.534 | 1.00 | 11.15 | C |
| ATOM | 3158 | C | ILE | C | 60 | 26.696 | 66.574 | 57.504 | 1.00 | 8.48 | C |
| ATOM | 3159 | O | ILE | C | 60 | 25.755 | 66.807 | 58.261 | 1.00 | 8.82 | C |
| ATOM | 3160 | N | TRP | C | 61 | 26.638 | 65.701 | 56.504 | 1.00 | 8.13 | C |
| ATOM | 3161 | CA | TRP | C | 61 | 25.443 | 64.912 | 56.217 | 1.00 | 8.40 | C |
| ATOM | 3162 | CB | TRP | C | 61 | 25.042 | 65.051 | 54.744 | 1.00 | 7.13 | C |
| ATOM | 3163 | CG | TRP | C | 61 | 24.380 | 66.349 | 54.417 | 1.00 | 7.30 | C |
| ATOM | 3164 | CD2 | TRP | C | 61 | 23.029 | 66.531 | 53.978 | 1.00 | 7.95 | C |
| ATOM | 3165 | CE2 | TRP | C | 61 | 22.827 | 67.919 | 53.813 | 1.00 | 8.17 | C |
| ATOM | 3166 | CE3 | TRP | C | 61 | 21.969 | 65.655 | 53.707 | 1.00 | 8.30 | C |
| ATOM | 3167 | CD1 | TRP | C | 61 | 24.929 | 67.594 | 54.499 | 1.00 | 8.73 | C |
| ATOM | 3168 | NE1 | TRP | C | 61 | 24.001 | 68.544 | 54.137 | 1.00 | 7.87 | C |
| ATOM | 3169 | CZ2 | TRP | C | 61 | 21.603 | 68.457 | 53.389 | 1.00 | 8.65 | C |
| ATOM | 3170 | CZ3 | TRP | C | 61 | 20.749 | 66.189 | 53.285 | 1.00 | 9.09 | C |
| ATOM | 3171 | CH2 | TRP | C | 61 | 20.581 | 67.580 | 53.131 | 1.00 | 9.32 | C |
| ATOM | 3172 | C | TRP | C | 61 | 25.702 | 63.442 | 56.517 | 1.00 | 9.89 | C |
| ATOM | 3173 | O | TRP | C | 61 | 26.593 | 62.827 | 55.929 | 1.00 | 9.58 | C |
| ATOM | 3174 | N | ASP | C | 62 | 24.920 | 62.885 | 57.435 | 1.00 | 9.03 | C |
| ATOM | 3175 | CA | ASP | C | 62 | 25.050 | 61.481 | 57.808 | 1.00 | 10.58 | C |
| ATOM | 3176 | CB | ASP | C | 62 | 24.945 | 61.335 | 59.333 | 1.00 | 9.78 | C |
| ATOM | 3177 | CG | ASP | C | 62 | 25.093 | 59.892 | 59.804 | 1.00 | 13.61 | C |
| ATOM | 3178 | OD1 | ASP | C | 62 | 25.747 | 59.087 | 59.107 | 1.00 | 12.65 | C |
| ATOM | 3179 | OD2 | ASP | C | 62 | 24.561 | 59.572 | 60.891 | 1.00 | 14.63 | C |
| ATOM | 3180 | C | ASP | C | 62 | 23.910 | 60.746 | 57.119 | 1.00 | 9.56 | C |
| ATOM | 3181 | O | ASP | C | 62 | 22.784 | 61.241 | 57.092 | 1.00 | 8.97 | C |
| ATOM | 3182 | N | THR | C | 63 | 24.202 | 59.578 | 56.554 | 1.00 | 10.44 | C |
| ATOM | 3183 | CA | THR | C | 63 | 23.183 | 58.804 | 55.859 | 1.00 | 11.33 | C |
| ATOM | 3184 | CB | THR | C | 63 | 23.414 | 58.816 | 54.328 | 1.00 | 12.10 | C |
| ATOM | 3185 | OG1 | THR | C | 63 | 24.640 | 58.135 | 54.018 | 1.00 | 12.84 | C |
| ATOM | 3186 | CG2 | THR | C | 63 | 23.500 | 60.245 | 53.814 | 1.00 | 12.77 | C |
| ATOM | 3187 | C | THR | C | 63 | 23.163 | 57.354 | 56.318 | 1.00 | 11.20 | C |
| ATOM | 3188 | O | THR | C | 63 | 24.183 | 56.807 | 56.735 | 1.00 | 12.66 | C |
| ATOM | 3189 | N | ALA | C | 64 | 21.989 | 56.739 | 56.253 | 1.00 | 10.70 | C |
| ATOM | 3190 | CA | ALA | C | 64 | 21.842 | 55.338 | 56.627 | 1.00 | 11.32 | C |
| ATOM | 3191 | CB | ALA | C | 64 | 20.368 | 54.991 | 56.769 | 1.00 | 12.84 | C |
| ATOM | 3192 | C | ALA | C | 64 | 22.478 | 54.537 | 55.488 | 1.00 | 11.47 | C |
| ATOM | 3193 | O | ALA | C | 64 | 22.123 | 54.716 | 54.323 | 1.00 | 12.95 | C |
| ATOM | 3194 | N | GLY | C | 65 | 23.405 | 53.649 | 55.830 | 1.00 | 11.19 | C |
| ATOM | 3195 | CA | GLY | C | 65 | 24.110 | 52.882 | 54.816 | 1.00 | 11.41 | C |
| ATOM | 3196 | C | GLY | C | 65 | 23.475 | 51.642 | 54.219 | 1.00 | 12.37 | C |
| ATOM | 3197 | O | GLY | C | 65 | 23.896 | 51.200 | 53.148 | 1.00 | 11.09 | C |
| ATOM | 3198 | N | GLN | C | 66 | 22.475 | 51.072 | 54.884 | 1.00 | 13.21 | C |
| ATOM | 3199 | CA | GLN | C | 66 | 21.837 | 49.871 | 54.358 | 1.00 | 14.67 | C |
| ATOM | 3200 | CB | GLN | C | 66 | 20.828 | 49.319 | 55.367 | 1.00 | 17.30 | C |
| ATOM | 3201 | CG | GLN | C | 66 | 21.487 | 48.812 | 56.644 | 1.00 | 19.83 | C |
| ATOM | 3202 | CD | GLN | C | 66 | 20.501 | 48.166 | 57.599 | 1.00 | 22.73 | C |
| ATOM | 3203 | OE1 | GLN | C | 66 | 19.573 | 48.813 | 58.085 | 1.00 | 25.04 | C |
| ATOM | 3204 | NE2 | GLN | C | 66 | 20.697 | 46.883 | 57.870 | 1.00 | 26.41 | C |
| ATOM | 3205 | C | GLN | C | 66 | 21.173 | 50.116 | 53.009 | 1.00 | 15.50 | C |
| ATOM | 3206 | O | GLN | C | 66 | 20.684 | 51.213 | 52.725 | 1.00 | 14.33 | C |
| ATOM | 3207 | N | GLU | C | 67 | 21.159 | 49.076 | 52.182 | 1.00 | 15.14 | C |
| ATOM | 3208 | CA | GLU | C | 67 | 20.590 | 49.145 | 50.841 | 1.00 | 16.14 | C |
| ATOM | 3209 | CB | GLU | C | 67 | 20.618 | 47.754 | 50.195 | 1.00 | 16.94 | C |
| ATOM | 3210 | CG | GLU | C | 67 | 22.013 | 47.173 | 50.034 | 0.00 | 17.54 | C |
| ATOM | 3211 | CD | GLU | C | 67 | 22.003 | 45.807 | 49.376 | 0.00 | 18.04 | C |
| ATOM | 3212 | OE1 | GLU | C | 67 | 21.525 | 45.702 | 48.227 | 0.00 | 18.36 | C |
| ATOM | 3213 | OE2 | GLU | C | 67 | 22.472 | 44.837 | 50.009 | 0.00 | 18.36 | C |
| ATOM | 3214 | C | GLU | C | 67 | 19.177 | 49.716 | 50.743 | 1.00 | 16.64 | C |
| ATOM | 3215 | O | GLU | C | 67 | 18.882 | 50.486 | 49.832 | 1.00 | 16.06 | C |
| ATOM | 3216 | N | ARG | C | 68 | 18.301 | 49.342 | 51.669 | 1.00 | 17.55 | C |
| ATOM | 3217 | CA | ARG | C | 68 | 16.924 | 49.822 | 51.619 | 1.00 | 19.64 | C |
| ATOM | 3218 | CB | ARG | C | 68 | 16.056 | 49.079 | 52.636 | 1.00 | 21.57 | C |
| ATOM | 3219 | CG | ARG | C | 68 | 16.507 | 49.213 | 54.074 | 1.00 | 24.19 | C |
| ATOM | 3220 | CD | ARG | C | 68 | 15.334 | 48.972 | 55.011 | 1.00 | 27.14 | C |
| ATOM | 3221 | NE | ARG | C | 68 | 15.729 | 48.286 | 56.237 | 1.00 | 30.20 | C |
| ATOM | 3222 | CZ | ARG | C | 68 | 16.140 | 47.023 | 56.280 | 1.00 | 31.79 | C |
| ATOM | 3223 | NH1 | ARG | C | 68 | 16.209 | 46.310 | 55.162 | 1.00 | 32.97 | C |
| ATOM | 3224 | NH2 | ARG | C | 68 | 16.477 | 46.469 | 57.437 | 1.00 | 30.76 | C |
| ATOM | 3225 | C | ARG | C | 68 | 16.757 | 51.325 | 51.820 | 1.00 | 20.76 | C |
| ATOM | 3226 | O | ARG | C | 68 | 15.652 | 51.850 | 51.669 | 1.00 | 21.43 | C |
| ATOM | 3227 | N | PHE | C | 69 | 17.839 | 52.020 | 52.157 | 1.00 | 19.81 | C |
| ATOM | 3228 | CA | PHE | C | 69 | 17.763 | 53.466 | 52.357 | 1.00 | 18.99 | C |
| ATOM | 3229 | CB | PHE | C | 69 | 18.543 | 53.886 | 53.608 | 1.00 | 21.26 | C |
| ATOM | 3230 | CG | PHE | C | 69 | 17.952 | 53.384 | 54.894 | 1.00 | 24.37 | C |
| ATOM | 3231 | CD1 | PHE | C | 69 | 18.054 | 52.044 | 55.247 | 1.00 | 24.96 | C |
| ATOM | 3232 | CD2 | PHE | C | 69 | 17.289 | 54.256 | 55.755 | 1.00 | 27.16 | C |
| ATOM | 3233 | CE1 | PHE | C | 69 | 17.506 | 51.575 | 56.438 | 1.00 | 26.28 | C |
| ATOM | 3234 | CE2 | PHE | C | 69 | 16.737 | 53.798 | 56.951 | 1.00 | 27.78 | C |
| ATOM | 3235 | CZ | PHE | C | 69 | 16.847 | 52.455 | 57.293 | 1.00 | 28.60 | C |
| ATOM | 3236 | C | PHE | C | 69 | 18.306 | 54.233 | 51.157 | 1.00 | 17.72 | C |
| ATOM | 3237 | O | PHE | C | 69 | 18.475 | 55.448 | 51.216 | 1.00 | 17.51 | C |
| ATOM | 3238 | N | ARG | C | 70 | 18.574 | 53.525 | 50.066 | 1.00 | 17.69 | C |
| ATOM | 3239 | CA | ARG | C | 70 | 19.115 | 54.166 | 48.873 | 1.00 | 15.96 | C |
| ATOM | 3240 | CB | ARG | C | 70 | 19.426 | 53.106 | 47.814 | 1.00 | 17.55 | C |
| ATOM | 3241 | CG | ARG | C | 70 | 20.102 | 53.631 | 46.562 | 1.00 | 19.74 | C |
| ATOM | 3242 | CD | ARG | C | 70 | 20.508 | 52.461 | 45.685 | 1.00 | 23.63 | C |
| ATOM | 3243 | NE | ARG | C | 70 | 21.424 | 51.575 | 46.399 | 1.00 | 26.26 | C |
| ATOM | 3244 | CZ | ARG | C | 70 | 21.435 | 50.251 | 46.281 | 1.00 | 27.09 | C |
| ATOM | 3245 | NH1 | ARG | C | 70 | 20.574 | 49.647 | 45.473 | 1.00 | 29.10 | C |
| ATOM | 3246 | NH2 | ARG | C | 70 | 22.306 | 49.532 | 46.978 | 1.00 | 26.71 | C |
| ATOM | 3247 | C | ARG | C | 70 | 18.192 | 55.238 | 48.290 | 1.00 | 14.77 | C |
| ATOM | 3248 | O | ARG | C | 70 | 18.638 | 56.340 | 47.982 | 1.00 | 12.69 | C |
| ATOM | 3249 | N | SER | C | 71 | 16.906 | 54.927 | 48.145 | 1.00 | 15.37 | C |
| ATOM | 3250 | CA | SER | C | 71 | 15.974 | 55.899 | 47.586 | 1.00 | 15.74 | C |
| ATOM | 3251 | CB | SER | C | 71 | 14.607 | 55.252 | 47.327 | 1.00 | 17.82 | C |
| ATOM | 3252 | OG | SER | C | 71 | 14.058 | 54.716 | 48.516 | 1.00 | 23.54 | C |
| ATOM | 3253 | C | SER | C | 71 | 15.813 | 57.121 | 48.485 | 1.00 | 14.87 | C |
| ATOM | 3254 | O | SER | C | 71 | 15.508 | 58.209 | 48.012 | 1.00 | 16.04 | C |
| ATOM | 3255 | N | LEU | C | 72 | 16.025 | 56.945 | 49.783 | 1.00 | 13.03 | C |
| ATOM | 3256 | CA | LEU | C | 72 | 15.900 | 58.059 | 50.713 | 1.00 | 10.23 | C |
| ATOM | 3257 | CB | LEU | C | 72 | 15.822 | 57.552 | 52.156 | 1.00 | 9.46 | C |
| ATOM | 3258 | CG | LEU | C | 72 | 15.959 | 58.600 | 53.271 | 1.00 | 9.31 | C |
| ATOM | 3259 | CD1 | LEU | C | 72 | 14.771 | 59.541 | 53.240 | 1.00 | 11.15 | C |
| ATOM | 3260 | CD2 | LEU | C | 72 | 16.043 | 57.907 | 54.629 | 1.00 | 11.40 | C |
| ATOM | 3261 | C | LEU | C | 72 | 17.072 | 59.019 | 50.599 | 1.00 | 10.37 | C |
| ATOM | 3262 | O | LEU | C | 72 | 16.880 | 60.232 | 50.502 | 1.00 | 11.65 | C |
| ATOM | 3263 | N | ARG | C | 73 | 18.284 | 58.468 | 50.593 | 1.00 | 8.21 | C |
| ATOM | 3264 | CA | ARG | C | 73 | 19.489 | 59.289 | 50.558 | 1.00 | 9.32 | C |
| ATOM | 3265 | CB | ARG | C | 73 | 20.619 | 58.570 | 51.310 | 1.00 | 7.45 | C |
| ATOM | 3266 | CG | ARG | C | 73 | 21.038 | 57.254 | 50.665 | 1.00 | 8.56 | C |
| ATOM | 3267 | CD | ARG | C | 73 | 22.298 | 56.631 | 51.277 | 1.00 | 9.09 | C |
| ATOM | 3268 | NE | ARG | C | 73 | 22.765 | 55.555 | 50.404 | 1.00 | 8.95 | C |
| ATOM | 3269 | CZ | ARG | C | 73 | 22.534 | 54.260 | 50.598 | 1.00 | 9.07 | C |
| ATOM | 3270 | NH1 | ARG | C | 73 | 21.858 | 53.842 | 51.663 | 1.00 | 7.76 | C |
| ATOM | 3271 | NH2 | ARG | C | 73 | 22.925 | 53.383 | 49.681 | 1.00 | 7.60 | C |
| ATOM | 3272 | C | ARG | C | 73 | 20.043 | 59.790 | 49.222 | 1.00 | 7.72 | C |
| ATOM | 3273 | O | ARG | C | 73 | 20.587 | 60.888 | 49.179 | 1.00 | 9.84 | C |
| ATOM | 3274 | N | THR | C | 74 | 19.913 | 59.022 | 48.139 | 1.00 | 9.41 | C |
| ATOM | 3275 | CA | THR | C | 74 | 20.506 | 59.454 | 46.864 | 1.00 | 9.39 | C |
| ATOM | 3276 | CB | THR | C | 74 | 20.309 | 58.401 | 45.727 | 1.00 | 10.56 | C |
| ATOM | 3277 | OG1 | THR | C | 74 | 18.920 | 58.112 | 45.546 | 1.00 | 12.61 | C |
| ATOM | 3278 | CG2 | THR | C | 74 | 21.055 | 57.117 | 46.064 | 1.00 | 11.41 | C |
| ATOM | 3279 | C | THR | C | 74 | 20.083 | 60.829 | 46.359 | 1.00 | 10.15 | C |
| ATOM | 3280 | O | THR | C | 74 | 20.894 | 61.565 | 45.797 | 1.00 | 9.64 | C |
| ATOM | 3281 | N | PRO | C | 75 | 18.813 | 61.205 | 46.549 | 1.00 | 11.62 | C |
| ATOM | 3282 | CD | PRO | C | 75 | 17.628 | 60.449 | 46.985 | 1.00 | 11.54 | C |
| ATOM | 3283 | CA | PRO | C | 75 | 18.446 | 62.535 | 46.058 | 1.00 | 12.14 | C |
| ATOM | 3284 | CB | PRO | C | 75 | 16.924 | 62.578 | 46.259 | 1.00 | 12.76 | C |
| ATOM | 3285 | CG | PRO | C | 75 | 16.662 | 61.548 | 47.311 | 1.00 | 16.80 | C |
| ATOM | 3286 | C | PRO | C | 75 | 19.181 | 63.672 | 46.779 | 1.00 | 11.31 | C |
| ATOM | 3287 | O | PRO | C | 75 | 19.163 | 64.814 | 46.324 | 1.00 | 10.87 | C |
| ATOM | 3288 | N | PHE | C | 76 | 19.841 | 63.363 | 47.892 | 1.00 | 9.71 | C |
| ATOM | 3289 | CA | PHE | C | 76 | 20.561 | 64.392 | 48.631 | 1.00 | 10.82 | C |
| ATOM | 3290 | CB | PHE | C | 76 | 20.197 | 64.319 | 50.115 | 1.00 | 12.46 | C |
| ATOM | 3291 | CG | PHE | C | 76 | 18.737 | 64.515 | 50.368 | 1.00 | 14.58 | C |
| ATOM | 3292 | CD1 | PHE | C | 76 | 17.861 | 63.434 | 50.346 | 1.00 | 14.84 | C |
| ATOM | 3293 | CD2 | PHE | C | 76 | 18.220 | 65.796 | 50.534 | 1.00 | 15.39 | C |
| ATOM | 3294 | CE1 | PHE | C | 76 | 16.486 | 63.628 | 50.481 | 1.00 | 15.10 | C |
| ATOM | 3295 | CE2 | PHE | C | 76 | 16.849 | 66.000 | 50.669 | 1.00 | 16.06 | C |
| ATOM | 3296 | CZ | PHE | C | 76 | 15.982 | 64.913 | 50.641 | 1.00 | 15.38 | C |
| ATOM | 3297 | C | PHE | C | 76 | 22.074 | 64.377 | 48.445 | 1.00 | 11.00 | C |
| ATOM | 3298 | O | PHE | C | 76 | 22.802 | 65.069 | 49.158 | 1.00 | 10.59 | C |
| ATOM | 3299 | N | TYR | C | 77 | 22.543 | 63.589 | 47.483 | 1.00 | 10.19 | C |
| ATOM | 3300 | CA | TYR | C | 77 | 23.967 | 63.526 | 47.177 | 1.00 | 11.32 | C |
| ATOM | 3301 | CB | TYR | C | 77 | 24.265 | 62.345 | 46.246 | 1.00 | 10.65 | C |
| ATOM | 3302 | CG | TYR | C | 77 | 24.279 | 60.979 | 46.895 | 1.00 | 10.95 | C |
| ATOM | 3303 | CD1 | TYR | C | 77 | 23.802 | 60.781 | 48.194 | 1.00 | 9.45 | C |
| ATOM | 3304 | CE1 | TYR | C | 77 | 23.789 | 59.505 | 48.772 | 1.00 | 9.62 | C |
| ATOM | 3305 | CD2 | TYR | C | 77 | 24.745 | 59.868 | 46.192 | 1.00 | 10.59 | C |
| ATOM | 3306 | CE2 | TYR | C | 77 | 24.735 | 58.597 | 46.757 | 1.00 | 12.60 | C |
| ATOM | 3307 | CZ | TYR | C | 77 | 24.257 | 58.419 | 48.045 | 1.00 | 10.61 | C |
| ATOM | 3308 | OH | TYR | C | 77 | 24.244 | 57.152 | 48.591 | 1.00 | 11.79 | C |
| ATOM | 3309 | C | TYR | C | 77 | 24.353 | 64.822 | 46.468 | 1.00 | 12.51 | C |
| ATOM | 3310 | O | TYR | C | 77 | 25.440 | 65.368 | 46.675 | 1.00 | 11.93 | C |
| ATOM | 3311 | N | ARG | C | 78 | 23.451 | 65.307 | 45.621 | 1.00 | 13.96 | C |
| ATOM | 3312 | CA | ARG | C | 78 | 23.684 | 66.535 | 44.872 | 1.00 | 15.46 | C |
| ATOM | 3313 | CB | ARG | C | 78 | 22.453 | 66.857 | 44.018 | 1.00 | 17.94 | C |
| ATOM | 3314 | CG | ARG | C | 78 | 22.603 | 68.083 | 43.134 | 1.00 | 22.41 | C |
| ATOM | 3315 | CD | ARG | C | 78 | 21.554 | 68.091 | 42.033 | 1.00 | 27.67 | C |
| ATOM | 3316 | NE | ARG | C | 78 | 20.205 | 67.918 | 42.562 | 1.00 | 31.14 | C |
| ATOM | 3317 | CZ | ARG | C | 78 | 19.618 | 68.761 | 43.407 | 1.00 | 34.24 | C |
| ATOM | 3318 | NH1 | ARG | C | 78 | 20.262 | 69.845 | 43.824 | 1.00 | 34.80 | C |
| ATOM | 3319 | NH2 | ARG | C | 78 | 18.385 | 68.521 | 43.835 | 1.00 | 33.41 | C |
| ATOM | 3320 | C | ARG | C | 78 | 24.002 | 67.700 | 45.806 | 1.00 | 15.15 | C |
| ATOM | 3321 | O | ARG | C | 78 | 23.399 | 67.835 | 46.872 | 1.00 | 15.43 | C |
| ATOM | 3322 | N | GLY | C | 79 | 24.966 | 68.528 | 45.407 | 1.00 | 14.40 | C |
| ATOM | 3323 | CA | GLY | C | 79 | 25.350 | 69.673 | 46.214 | 1.00 | 14.00 | C |
| ATOM | 3324 | C | GLY | C | 79 | 26.539 | 69.408 | 47.123 | 1.00 | 12.61 | C |
| ATOM | 3325 | O | GLY | C | 79 | 27.142 | 70.338 | 47.669 | 1.00 | 12.61 | C |
| ATOM | 3326 | N | SER | C | 80 | 26.879 | 68.136 | 47.291 | 1.00 | 11.04 | C |
| ATOM | 3327 | CA | SER | C | 80 | 28.000 | 67.763 | 48.144 | 1.00 | 11.61 | C |
| ATOM | 3328 | CB | SER | C | 80 | 28.079 | 66.240 | 48.280 | 1.00 | 11.45 | C |
| ATOM | 3329 | OG | SER | C | 80 | 26.888 | 65.707 | 48.836 | 1.00 | 11.60 | C |
| ATOM | 3330 | C | SER | C | 80 | 29.307 | 68.293 | 47.568 | 1.00 | 11.71 | C |
| ATOM | 3331 | O | SER | C | 80 | 29.515 | 68.279 | 46.353 | 1.00 | 10.79 | C |
| ATOM | 3332 | N | ASP | C | 81 | 30.187 | 68.759 | 48.444 | 1.00 | 11.80 | C |
| ATOM | 3333 | CA | ASP | C | 81 | 31.475 | 69.289 | 48.019 | 1.00 | 13.88 | C |
| ATOM | 3334 | CB | ASP | C | 81 | 31.779 | 70.586 | 48.769 | 1.00 | 15.29 | C |
| ATOM | 3335 | CG | ASP | C | 81 | 30.836 | 71.709 | 48.387 | 1.00 | 18.79 | C |
| ATOM | 3336 | OD1 | ASP | C | 81 | 30.816 | 72.078 | 47.193 | 1.00 | 18.78 | C |
| ATOM | 3337 | OD2 | ASP | C | 81 | 30.112 | 72.218 | 49.270 | 1.00 | 18.63 | C |
| ATOM | 3338 | C | ASP | C | 81 | 32.575 | 68.265 | 48.264 | 1.00 | 14.23 | C |
| ATOM | 3339 | O | ASP | C | 81 | 33.654 | 68.335 | 47.674 | 1.00 | 13.93 | C |
| ATOM | 3340 | N | CYS | C | 82 | 32.295 | 67.313 | 49.146 | 1.00 | 13.10 | C |
| ATOM | 3341 | CA | CYS | C | 82 | 33.253 | 66.264 | 49.463 | 1.00 | 13.77 | C |
| ATOM | 3342 | CB | CYS | C | 82 | 34.273 | 66.748 | 50.490 | 1.00 | 14.67 | C |
| ATOM | 3343 | SG | CYS | C | 82 | 35.522 | 65.500 | 50.909 | 1.00 | 19.14 | C |
| ATOM | 3344 | C | CYS | C | 82 | 32.515 | 65.063 | 50.023 | 1.00 | 13.88 | C |
| ATOM | 3345 | O | CYS | C | 82 | 31.470 | 65.207 | 50.659 | 1.00 | 12.05 | C |
| ATOM | 3346 | N | CYS | C | 83 | 33.057 | 63.879 | 49.777 | 1.00 | 12.46 | C |
| ATOM | 3347 | CA | CYS | C | 83 | 32.443 | 62.657 | 50.270 | 1.00 | 12.43 | C |
| ATOM | 3348 | CB | CYS | C | 83 | 31.988 | 61.784 | 49.100 | 1.00 | 12.40 | C |
| ATOM | 3349 | SG | CYS | C | 83 | 31.401 | 60.138 | 49.580 | 1.00 | 16.95 | C |
| ATOM | 3350 | C | CYS | C | 83 | 33.421 | 61.884 | 51.138 | 1.00 | 13.40 | C |
| ATOM | 3351 | O | CYS | C | 83 | 34.522 | 61.540 | 50.700 | 1.00 | 12.83 | C |
| ATOM | 3352 | N | LEU | C | 84 | 33.021 | 61.632 | 52.379 | 1.00 | 12.30 | C |
| ATOM | 3353 | CA | LEU | C | 84 | 33.843 | 60.872 | 53.308 | 1.00 | 12.59 | C |
| ATOM | 3354 | CB | LEU | C | 84 | 33.653 | 61.384 | 54.736 | 1.00 | 15.11 | C |
| ATOM | 3355 | CG | LEU | C | 84 | 34.325 | 62.713 | 55.078 | 1.00 | 14.27 | C |
| ATOM | 3356 | CD1 | LEU | C | 84 | 33.866 | 63.177 | 56.446 | 1.00 | 19.65 | C |
| ATOM | 3357 | CD2 | LEU | C | 84 | 35.834 | 62.546 | 55.056 | 1.00 | 18.18 | C |
| ATOM | 3358 | C | LEU | C | 84 | 33.386 | 59.428 | 53.207 | 1.00 | 13.83 | C |
| ATOM | 3359 | O | LEU | C | 84 | 32.428 | 59.025 | 53.870 | 1.00 | 15.98 | C |
| ATOM | 3360 | N | LEU | C | 85 | 34.060 | 58.663 | 52.354 | 1.00 | 12.89 | C |
| ATOM | 3361 | CA | LEU | C | 85 | 33.729 | 57.261 | 52.144 | 1.00 | 11.49 | C |
| ATOM | 3362 | CB | LEU | C | 85 | 34.298 | 56.789 | 50.810 | 1.00 | 15.49 | C |
| ATOM | 3363 | CG | LEU | C | 85 | 33.545 | 55.636 | 50.152 | 1.00 | 20.38 | C |
| ATOM | 3364 | CD1 | LEU | C | 85 | 32.063 | 55.990 | 50.047 | 1.00 | 21.42 | C |
| ATOM | 3365 | CD2 | LEU | C | 85 | 34.128 | 55.368 | 48.774 | 1.00 | 20.59 | C |
| ATOM | 3366 | C | LEU | C | 85 | 34.322 | 56.468 | 53.298 | 1.00 | 9.44 | C |
| ATOM | 3367 | O | LEU | C | 85 | 35.535 | 56.426 | 53.480 | 1.00 | 9.45 | C |
| ATOM | 3368 | N | THR | C | 86 | 33.457 | 55.828 | 54.074 | 1.00 | 7.99 | C |
| ATOM | 3369 | CA | THR | C | 86 | 33.912 | 55.095 | 55.246 | 1.00 | 7.97 | C |
| ATOM | 3370 | CB | THR | C | 86 | 33.219 | 55.650 | 56.508 | 1.00 | 10.75 | C |
| ATOM | 3371 | OG1 | THR | C | 86 | 33.423 | 57.065 | 56.579 | 1.00 | 13.81 | C |
| ATOM | 3372 | CG2 | THR | C | 86 | 33.766 | 54.989 | 57.763 | 1.00 | 10.47 | C |
| ATOM | 3373 | C | THR | C | 86 | 33.686 | 53.592 | 55.235 | 1.00 | 7.08 | C |
| ATOM | 3374 | O | THR | C | 86 | 32.656 | 53.112 | 54.769 | 1.00 | 6.31 | C |
| ATOM | 3375 | N | PHE | C | 87 | 34.667 | 52.856 | 55.750 | 1.00 | 6.52 | C |
| ATOM | 3376 | CA | PHE | C | 87 | 34.552 | 51.408 | 55.889 | 1.00 | 6.93 | C |
| ATOM | 3377 | CB | PHE | C | 87 | 35.332 | 50.647 | 54.795 | 1.00 | 6.92 | C |
| ATOM | 3378 | CG | PHE | C | 87 | 36.836 | 50.741 | 54.911 | 1.00 | 8.43 | C |
| ATOM | 3379 | CD1 | PHE | C | 87 | 37.530 | 51.816 | 54.363 | 1.00 | 9.25 | C |
| ATOM | 3380 | CD2 | PHE | C | 87 | 37.561 | 49.732 | 55.544 | 1.00 | 7.48 | C |
| ATOM | 3381 | CE1 | PHE | C | 87 | 38.928 | 51.886 | 54.438 | 1.00 | 8.53 | C |
| ATOM | 3382 | CE2 | PHE | C | 87 | 38.956 | 49.794 | 55.626 | 1.00 | 7.63 | C |
| ATOM | 3383 | CZ | PHE | C | 87 | 39.639 | 50.873 | 55.069 | 1.00 | 8.76 | C |
| ATOM | 3384 | C | PHE | C | 87 | 35.105 | 51.079 | 57.271 | 1.00 | 7.44 | C |
| ATOM | 3385 | O | PHE | C | 87 | 35.670 | 51.950 | 57.941 | 1.00 | 8.23 | C |
| ATOM | 3386 | N | SER | C | 88 | 34.916 | 49.839 | 57.715 | 1.00 | 6.97 | C |
| ATOM | 3387 | CA | SER | C | 88 | 35.415 | 49.407 | 59.015 | 1.00 | 7.82 | C |
| ATOM | 3388 | CB | SER | C | 88 | 34.313 | 48.686 | 59.798 | 1.00 | 9.43 | C |
| ATOM | 3389 | OG | SER | C | 88 | 34.860 | 47.944 | 60.876 | 1.00 | 12.86 | C |
| ATOM | 3390 | C | SER | C | 88 | 36.573 | 48.452 | 58.764 | 1.00 | 6.79 | C |
| ATOM | 3391 | O | SER | C | 88 | 36.436 | 47.517 | 57.985 | 1.00 | 9.21 | C |
| ATOM | 3392 | N | VAL | C | 89 | 37.710 | 48.682 | 59.412 | 1.00 | 8.12 | C |
| ATOM | 3393 | CA | VAL | C | 89 | 38.867 | 47.811 | 59.205 | 1.00 | 8.73 | C |
| ATOM | 3394 | CB | VAL | C | 89 | 40.154 | 48.408 | 59.825 | 1.00 | 9.71 | C |
| ATOM | 3395 | CG1 | VAL | C | 89 | 40.404 | 49.793 | 59.260 | 1.00 | 10.58 | C |
| ATOM | 3396 | CG2 | VAL | C | 89 | 40.043 | 48.451 | 61.347 | 1.00 | 9.50 | C |
| ATOM | 3397 | C | VAL | C | 89 | 38.635 | 46.424 | 59.788 | 1.00 | 10.57 | C |
| ATOM | 3398 | O | VAL | C | 89 | 39.420 | 45.502 | 59.554 | 1.00 | 9.66 | C |
| ATOM | 3399 | N | ASP | C | 90 | 37.548 | 46.282 | 60.538 | 1.00 | 11.03 | C |
| ATOM | 3400 | CA | ASP | C | 90 | 37.190 | 45.009 | 61.159 | 1.00 | 14.82 | C |
| ATOM | 3401 | CB | ASP | C | 90 | 36.636 | 45.276 | 62.564 | 1.00 | 17.98 | C |
| ATOM | 3402 | CG | ASP | C | 90 | 36.539 | 44.020 | 63.413 | 1.00 | 23.21 | C |
| ATOM | 3403 | OD1 | ASP | C | 90 | 37.562 | 43.315 | 63.564 | 1.00 | 24.60 | C |
| ATOM | 3404 | OD2 | ASP | C | 90 | 35.439 | 43.745 | 63.940 | 1.00 | 25.43 | C |
| ATOM | 3405 | C | ASP | C | 90 | 36.148 | 44.268 | 60.309 | 1.00 | 14.90 | C |
| ATOM | 3406 | O | ASP | C | 90 | 35.702 | 43.177 | 60.674 | 1.00 | 15.13 | C |
| ATOM | 3407 | N | ASP | C | 91 | 35.771 | 44.862 | 59.177 | 1.00 | 14.38 | C |
| ATOM | 3408 | CA | ASP | C | 91 | 34.767 | 44.282 | 58.280 | 1.00 | 15.68 | C |
| ATOM | 3409 | CB | ASP | C | 91 | 33.435 | 45.024 | 58.457 | 1.00 | 17.43 | C |
| ATOM | 3410 | CG | ASP | C | 91 | 32.362 | 44.557 | 57.487 | 1.00 | 19.35 | C |
| ATOM | 3411 | OD1 | ASP | C | 91 | 32.542 | 43.503 | 56.850 | 1.00 | 22.71 | C |
| ATOM | 3412 | OD2 | ASP | C | 91 | 31.325 | 45.248 | 57.371 | 1.00 | 24.90 | C |
| ATOM | 3413 | C | ASP | C | 91 | 35.215 | 44.349 | 56.822 | 1.00 | 14.04 | C |
| ATOM | 3414 | O | ASP | C | 91 | 35.018 | 45.360 | 56.146 | 1.00 | 13.18 | C |
| ATOM | 3415 | N | SER | C | 92 | 35.805 | 43.262 | 56.333 | 1.00 | 13.49 | C |
| ATOM | 3416 | CA | SER | C | 92 | 36.302 | 43.222 | 54.961 | 1.00 | 13.98 | C |
| ATOM | 3417 | CB | SER | C | 92 | 36.950 | 41.862 | 54.669 | 1.00 | 15.56 | C |
| ATOM | 3418 | OG | SER | C | 92 | 36.030 | 40.809 | 54.875 | 1.00 | 20.76 | C |
| ATOM | 3419 | C | SER | C | 92 | 35.249 | 43.526 | 53.900 | 1.00 | 13.28 | C |
| ATOM | 3420 | O | SER | C | 92 | 35.561 | 44.134 | 52.875 | 1.00 | 12.70 | C |
| ATOM | 3421 | N | GLN | C | 93 | 34.006 | 43.109 | 54.133 | 1.00 | 13.11 | C |
| ATOM | 3422 | CA | GLN | C | 93 | 32.952 | 43.369 | 53.157 | 1.00 | 14.77 | C |
| ATOM | 3423 | CB | GLN | C | 93 | 31.634 | 42.722 | 53.592 | 1.00 | 18.52 | C |
| ATOM | 3424 | CG | GLN | C | 93 | 30.543 | 42.809 | 52.534 | 1.00 | 24.45 | C |
| ATOM | 3425 | CD | GLN | C | 93 | 29.262 | 42.117 | 52.951 | 1.00 | 28.96 | C |
| ATOM | 3426 | OE1 | GLN | C | 93 | 28.564 | 42.564 | 53.867 | 1.00 | 31.16 | C |
| ATOM | 3427 | NE2 | GLN | C | 93 | 28.945 | 41.012 | 52.283 | 1.00 | 30.94 | C |
| ATOM | 3428 | C | GLN | C | 93 | 32.738 | 44.865 | 52.953 | 1.00 | 13.12 | C |
| ATOM | 3429 | O | GLN | C | 93 | 32.531 | 45.319 | 51.822 | 1.00 | 11.07 | C |
| ATOM | 3430 | N | SER | C | 94 | 32.793 | 45.632 | 54.039 | 1.00 | 10.43 | C |
| ATOM | 3431 | CA | SER | C | 94 | 32.593 | 47.074 | 53.944 | 1.00 | 9.20 | C |
| ATOM | 3432 | CB | SER | C | 94 | 32.575 | 47.718 | 55.339 | 1.00 | 9.43 | C |
| ATOM | 3433 | OG | SER | C | 94 | 33.831 | 47.629 | 55.996 | 1.00 | 9.23 | C |
| ATOM | 3434 | C | SER | C | 94 | 33.671 | 47.717 | 53.085 | 1.00 | 8.29 | C |
| ATOM | 3435 | O | SER | C | 94 | 33.416 | 48.699 | 52.394 | 1.00 | 7.78 | C |
| ATOM | 3436 | N | PHE | C | 95 | 34.876 | 47.157 | 53.129 | 1.00 | 7.29 | C |
| ATOM | 3437 | CA | PHE | C | 95 | 35.983 | 47.671 | 52.336 | 1.00 | 7.14 | C |
| ATOM | 3438 | CB | PHE | C | 95 | 37.311 | 47.090 | 52.831 | 1.00 | 6.61 | C |
| ATOM | 3439 | CG | PHE | C | 95 | 38.479 | 47.403 | 51.936 | 1.00 | 8.33 | C |
| ATOM | 3440 | CD1 | PHE | C | 95 | 38.905 | 48.715 | 51.762 | 1.00 | 7.62 | C |
| ATOM | 3441 | CD2 | PHE | C | 95 | 39.140 | 46.386 | 51.251 | 1.00 | 10.93 | C |
| ATOM | 3442 | CE1 | PHE | C | 95 | 39.979 | 49.016 | 50.914 | 1.00 | 9.75 | C |
| ATOM | 3443 | CE2 | PHE | C | 95 | 40.211 | 46.676 | 50.404 | 1.00 | 10.54 | C |
| ATOM | 3444 | CZ | PHE | C | 95 | 40.631 | 47.992 | 50.235 | 1.00 | 8.53 | C |
| ATOM | 3445 | C | PHE | C | 95 | 35.782 | 47.293 | 50.869 | 1.00 | 7.63 | C |
| ATOM | 3446 | O | PHE | C | 95 | 35.965 | 48.114 | 49.982 | 1.00 | 7.67 | C |
| ATOM | 3447 | N | GLN | C | 96 | 35.415 | 46.039 | 50.625 | 1.00 | 8.71 | C |
| ATOM | 3448 | CA | GLN | C | 96 | 35.189 | 45.555 | 49.269 | 1.00 | 9.84 | C |
| ATOM | 3449 | CB | GLN | C | 96 | 34.804 | 44.073 | 49.302 | 1.00 | 10.37 | C |
| ATOM | 3450 | CG | GLN | C | 96 | 35.959 | 43.130 | 49.613 | 1.00 | 14.28 | C |
| ATOM | 3451 | CD | GLN | C | 96 | 35.492 | 41.708 | 49.888 | 1.00 | 18.28 | C |
| ATOM | 3452 | OE1 | GLN | C | 96 | 34.646 | 41.169 | 49.175 | 1.00 | 21.42 | C |
| ATOM | 3453 | NE2 | GLN | C | 96 | 36.053 | 41.092 | 50.923 | 1.00 | 21.42 | C |
| ATOM | 3454 | C | GLN | C | 96 | 34.094 | 46.354 | 48.561 | 1.00 | 10.95 | C |
| ATOM | 3455 | O | GLN | C | 96 | 34.052 | 46.407 | 47.334 | 1.00 | 12.38 | C |
| ATOM | 3456 | N | ASN | C | 97 | 33.216 | 46.978 | 49.338 | 1.00 | 9.46 | C |
| ATOM | 3457 | CA | ASN | C | 97 | 32.117 | 47.761 | 48.778 | 1.00 | 10.74 | C |
| ATOM | 3458 | CB | ASN | C | 97 | 30.916 | 47.724 | 49.734 | 1.00 | 11.55 | C |
| ATOM | 3459 | CG | ASN | C | 97 | 30.197 | 46.378 | 49.733 | 1.00 | 15.19 | C |
| ATOM | 3460 | OD1 | ASN | C | 97 | 29.474 | 46.047 | 50.677 | 1.00 | 15.72 | C |
| ATOM | 3461 | ND2 | ASN | C | 97 | 30.376 | 45.605 | 48.665 | 1.00 | 12.77 | C |
| ATOM | 3462 | C | ASN | C | 97 | 32.466 | 49.222 | 48.455 | 1.00 | 10.75 | C |
| ATOM | 3463 | O | ASN | C | 97 | 31.605 | 49.973 | 47.998 | 1.00 | 9.03 | C |
| ATOM | 3464 | N | LEU | C | 98 | 33.715 | 49.630 | 48.670 | 1.00 | 10.89 | C |
| ATOM | 3465 | CA | LEU | C | 98 | 34.089 | 51.018 | 48.391 | 1.00 | 11.12 | C |
| ATOM | 3466 | CB | LEU | C | 98 | 35.543 | 51.289 | 48.804 | 1.00 | 10.52 | C |
| ATOM | 3467 | CG | LEU | C | 98 | 35.849 | 51.353 | 50.304 | 1.00 | 12.16 | C |
| ATOM | 3468 | CD1 | LEU | C | 98 | 37.329 | 51.655 | 50.509 | 1.00 | 11.37 | C |
| ATOM | 3469 | CD2 | LEU | C | 98 | 34.997 | 52.430 | 50.965 | 1.00 | 11.72 | C |
| ATOM | 3470 | C | LEU | C | 98 | 33.883 | 51.426 | 46.930 | 1.00 | 11.74 | C |
| ATOM | 3471 | O | LEU | C | 98 | 33.364 | 52.511 | 46.651 | 1.00 | 10.84 | C |
| ATOM | 3472 | N | SER | C | 99 | 34.291 | 50.572 | 45.996 | 1.00 | 12.12 | C |
| ATOM | 3473 | CA | SER | C | 99 | 34.125 | 50.895 | 44.585 | 1.00 | 11.07 | C |
| ATOM | 3474 | CB | SER | C | 99 | 34.681 | 49.777 | 43.699 | 1.00 | 14.17 | C |
| ATOM | 3475 | OG | SER | C | 99 | 36.102 | 49.775 | 43.717 | 1.00 | 16.87 | C |
| ATOM | 3476 | C | SER | C | 99 | 32.653 | 51.131 | 44.265 | 1.00 | 11.86 | C |
| ATOM | 3477 | O | SER | C | 99 | 32.321 | 52.061 | 43.533 | 1.00 | 10.98 | C |
| ATOM | 3478 | N | ASN | C | 100 | 31.773 | 50.298 | 44.817 | 1.00 | 12.44 | C |
| ATOM | 3479 | CA | ASN | C | 100 | 30.342 | 50.451 | 44.574 | 1.00 | 12.36 | C |
| ATOM | 3480 | CB | ASN | C | 100 | 29.552 | 49.264 | 45.134 | 1.00 | 14.14 | C |
| ATOM | 3481 | CG | ASN | C | 100 | 29.720 | 48.005 | 44.299 | 1.00 | 16.89 | C |
| ATOM | 3482 | OD1 | ASN | C | 100 | 29.921 | 48.074 | 43.084 | 1.00 | 19.63 | C |
| ATOM | 3483 | ND2 | ASN | C | 100 | 29.622 | 46.850 | 44.943 | 1.00 | 18.64 | C |
| ATOM | 3484 | C | ASN | C | 100 | 29.817 | 51.750 | 45.176 | 1.00 | 11.51 | C |
| ATOM | 3485 | O | ASN | C | 100 | 28.956 | 52.403 | 44.590 | 1.00 | 10.70 | C |
| ATOM | 3486 | N | TRP | C | 101 | 30.340 | 52.130 | 46.340 | 1.00 | 10.83 | C |
| ATOM | 3487 | CA | TRP | C | 101 | 29.907 | 53.370 | 46.973 | 1.00 | 8.74 | C |
| ATOM | 3488 | CB | TRP | C | 101 | 30.475 | 53.484 | 48.388 | 1.00 | 9.92 | C |
| ATOM | 3489 | CG | TRP | C | 101 | 29.633 | 52.776 | 49.402 | 1.00 | 10.13 | C |
| ATOM | 3490 | CD2 | TRP | C | 101 | 28.321 | 53.157 | 49.836 | 1.00 | 8.79 | C |
| ATOM | 3491 | CE2 | TRP | C | 101 | 27.894 | 52.193 | 50.776 | 1.00 | 9.15 | C |
| ATOM | 3492 | CE3 | TRP | C | 101 | 27.463 | 54.222 | 49.521 | 1.00 | 9.28 | C |
| ATOM | 3493 | CD1 | TRP | C | 101 | 29.942 | 51.628 | 50.077 | 1.00 | 9.75 | C |
| ATOM | 3494 | NE1 | TRP | C | 101 | 28.901 | 51.271 | 50.904 | 1.00 | 10.32 | C |
| ATOM | 3495 | CZ2 | TRP | C | 101 | 26.648 | 52.262 | 51.405 | 1.00 | 7.64 | C |
| ATOM | 3496 | CZ3 | TRP | C | 101 | 26.224 | 54.290 | 50.147 | 1.00 | 8.02 | C |
| ATOM | 3497 | CH2 | TRP | C | 101 | 25.830 | 53.315 | 51.079 | 1.00 | 8.97 | C |
| ATOM | 3498 | C | TRP | C | 101 | 30.325 | 54.575 | 46.141 | 1.00 | 8.82 | C |
| ATOM | 3499 | O | TRP | C | 101 | 29.540 | 55.504 | 45.946 | 1.00 | 7.50 | C |
| ATOM | 3500 | N | LYS | C | 102 | 31.562 | 54.564 | 45.649 | 1.00 | 7.37 | C |
| ATOM | 3501 | CA | LYS | C | 102 | 32.031 | 55.667 | 44.820 | 1.00 | 8.96 | C |
| ATOM | 3502 | CB | LYS | C | 102 | 33.483 | 55.448 | 44.385 | 1.00 | 9.22 | C |
| ATOM | 3503 | CG | LYS | C | 102 | 33.947 | 56.465 | 43.346 | 1.00 | 8.42 | C |
| ATOM | 3504 | CD | LYS | C | 102 | 35.402 | 56.287 | 42.944 | 1.00 | 9.89 | C |
| ATOM | 3505 | CE | LYS | C | 102 | 35.801 | 57.356 | 41.930 | 1.00 | 10.79 | C |
| ATOM | 3506 | NZ | LYS | C | 102 | 37.232 | 57.269 | 41.511 | 1.00 | 11.47 | C |
| ATOM | 3507 | C | LYS | C | 102 | 31.143 | 55.786 | 43.582 | 1.00 | 7.80 | C |
| ATOM | 3508 | O | LYS | C | 102 | 30.699 | 56.879 | 43.223 | 1.00 | 9.40 | C |
| ATOM | 3509 | N | LYS | C | 103 | 30.886 | 54.655 | 42.930 | 1.00 | 10.57 | C |
| ATOM | 3510 | CA | LYS | C | 103 | 30.051 | 54.639 | 41.732 | 1.00 | 11.20 | C |
| ATOM | 3511 | CB | LYS | C | 103 | 29.942 | 53.220 | 41.165 | 1.00 | 12.25 | C |
| ATOM | 3512 | CG | LYS | C | 103 | 31.246 | 52.675 | 40.598 | 1.00 | 17.46 | C |
| ATOM | 3513 | CD | LYS | C | 103 | 31.032 | 51.334 | 39.911 | 1.00 | 21.06 | C |
| ATOM | 3514 | CE | LYS | C | 103 | 32.357 | 50.697 | 39.502 | 1.00 | 24.04 | C |
| ATOM | 3515 | NZ | LYS | C | 103 | 33.144 | 51.580 | 38.598 | 1.00 | 28.25 | C |
| ATOM | 3516 | C | LYS | C | 103 | 28.657 | 55.175 | 42.015 | 1.00 | 11.94 | C |
| ATOM | 3517 | O | LYS | C | 103 | 28.117 | 55.954 | 41.230 | 1.00 | 10.95 | C |
| ATOM | 3518 | N | GLU | C | 104 | 28.069 | 54.758 | 43.133 | 1.00 | 10.99 | C |
| ATOM | 3519 | CA | GLU | C | 104 | 26.737 | 55.227 | 43.471 | 1.00 | 10.44 | C |
| ATOM | 3520 | CB | GLU | C | 104 | 26.208 | 54.530 | 44.727 | 1.00 | 9.77 | C |
| ATOM | 3521 | CG | GLU | C | 104 | 24.814 | 54.999 | 45.109 | 1.00 | 12.61 | C |
| ATOM | 3522 | CD | GLU | C | 104 | 24.222 | 54.222 | 46.263 | 1.00 | 13.76 | C |
| ATOM | 3523 | OE1 | GLU | C | 104 | 23.986 | 53.005 | 46.101 | 1.00 | 14.48 | C |
| ATOM | 3524 | OE2 | GLU | C | 104 | 23.990 | 54.831 | 47.329 | 1.00 | 15.00 | C |
| ATOM | 3525 | C | GLU | C | 104 | 26.737 | 56.735 | 43.677 | 1.00 | 10.08 | C |
| ATOM | 3526 | O | GLU | C | 104 | 25.826 | 57.426 | 43.224 | 1.00 | 10.91 | C |
| ATOM | 3527 | N | PHE | C | 105 | 27.758 | 57.259 | 44.350 | 1.00 | 10.66 | C |
| ATOM | 3528 | CA | PHE | C | 105 | 27.802 | 58.698 | 44.572 | 1.00 | 10.94 | C |
| ATOM | 3529 | CB | PHE | C | 105 | 29.026 | 59.113 | 45.387 | 1.00 | 9.96 | C |
| ATOM | 3530 | CG | PHE | C | 105 | 29.105 | 60.594 | 45.612 | 1.00 | 10.55 | C |
| ATOM | 3531 | CD1 | PHE | C | 105 | 28.263 | 61.219 | 46.532 | 1.00 | 10.44 | C |
| ATOM | 3532 | CD2 | PHE | C | 105 | 29.969 | 61.378 | 44.855 | 1.00 | 11.35 | C |
| ATOM | 3533 | CE1 | PHE | C | 105 | 28.279 | 62.604 | 46.692 | 1.00 | 11.48 | C |
| ATOM | 3534 | CE2 | PHE | C | 105 | 29.994 | 62.762 | 45.006 | 1.00 | 12.36 | C |
| ATOM | 3535 | CZ | PHE | C | 105 | 29.148 | 63.378 | 45.925 | 1.00 | 13.09 | C |
| ATOM | 3536 | C | PHE | C | 105 | 27.828 | 59.468 | 43.255 | 1.00 | 12.18 | C |
| ATOM | 3537 | O | PHE | C | 105 | 26.986 | 60.332 | 43.010 | 1.00 | 10.43 | C |
| ATOM | 3538 | N | ILE | C | 106 | 28.811 | 59.158 | 42.418 | 1.00 | 12.01 | C |
| ATOM | 3539 | CA | ILE | C | 106 | 28.960 | 59.829 | 41.133 | 1.00 | 12.22 | C |
| ATOM | 3540 | CB | ILE | C | 106 | 30.123 | 59.214 | 40.327 | 1.00 | 12.04 | C |
| ATOM | 3541 | CG2 | ILE | C | 106 | 30.157 | 59.797 | 38.917 | 1.00 | 12.30 | C |
| ATOM | 3542 | CG1 | ILE | C | 106 | 31.450 | 59.471 | 41.052 | 1.00 | 12.82 | C |
| ATOM | 3543 | CD1 | ILE | C | 106 | 31.784 | 60.945 | 41.252 | 1.00 | 13.05 | C |
| ATOM | 3544 | C | ILE | C | 106 | 27.681 | 59.755 | 40.310 | 1.00 | 12.26 | C |
| ATOM | 3545 | O | ILE | C | 106 | 27.269 | 60.743 | 39.701 | 1.00 | 12.63 | C |
| ATOM | 3546 | N | TYR | C | 107 | 27.044 | 58.590 | 40.310 | 1.00 | 12.76 | C |
| ATOM | 3547 | CA | TYR | C | 107 | 25.817 | 58.399 | 39.546 | 1.00 | 15.12 | C |
| ATOM | 3548 | CB | TYR | C | 107 | 25.309 | 56.965 | 39.699 | 1.00 | 17.46 | C |
| ATOM | 3549 | CG | TYR | C | 107 | 24.169 | 56.639 | 38.759 | 1.00 | 20.57 | C |
| ATOM | 3550 | CD1 | TYR | C | 107 | 24.396 | 56.470 | 37.393 | 1.00 | 22.38 | C |
| ATOM | 3551 | CE1 | TYR | C | 107 | 23.347 | 56.204 | 36.516 | 1.00 | 24.66 | C |
| ATOM | 3552 | CD2 | TYR | C | 107 | 22.859 | 56.533 | 39.229 | 1.00 | 22.88 | C |
| ATOM | 3553 | CE2 | TYR | C | 107 | 21.803 | 56.267 | 38.360 | 1.00 | 24.78 | C |
| ATOM | 3554 | CZ | TYR | C | 107 | 22.055 | 56.104 | 37.006 | 1.00 | 24.91 | C |
| ATOM | 3555 | OH | TYR | C | 107 | 21.017 | 55.844 | 36.142 | 1.00 | 27.80 | C |
| ATOM | 3556 | C | TYR | C | 107 | 24.697 | 59.360 | 39.938 | 1.00 | 15.37 | C |
| ATOM | 3557 | O | TYR | C | 107 | 24.058 | 59.957 | 39.074 | 1.00 | 14.77 | C |
| ATOM | 3558 | N | TYR | C | 108 | 24.469 | 59.513 | 41.239 | 1.00 | 14.31 | C |
| ATOM | 3559 | CA | TYR | C | 108 | 23.397 | 60.373 | 41.736 | 1.00 | 16.40 | C |
| ATOM | 3560 | CB | TYR | C | 108 | 22.750 | 59.724 | 42.959 | 1.00 | 15.75 | C |
| ATOM | 3561 | CG | TYR | C | 108 | 22.006 | 58.449 | 42.649 | 1.00 | 18.07 | C |
| ATOM | 3562 | CD1 | TYR | C | 108 | 20.757 | 58.486 | 42.026 | 1.00 | 18.66 | C |
| ATOM | 3563 | CE1 | TYR | C | 108 | 20.069 | 57.320 | 41.733 | 1.00 | 18.08 | C |
| ATOM | 3564 | CD2 | TYR | C | 108 | 22.549 | 57.207 | 42.969 | 1.00 | 16.69 | C |
| ATOM | 3565 | CE2 | TYR | C | 108 | 21.868 | 56.031 | 42.678 | 1.00 | 18.32 | C |
| ATOM | 3566 | CZ | TYR | C | 108 | 20.629 | 56.097 | 42.061 | 1.00 | 19.41 | C |
| ATOM | 3567 | OH | TYR | C | 108 | 19.949 | 54.941 | 41.773 | 1.00 | 20.10 | C |
| ATOM | 3568 | C | TYR | C | 108 | 23.765 | 61.807 | 42.089 | 1.00 | 15.58 | C |
| ATOM | 3569 | O | TYR | C | 108 | 22.892 | 62.672 | 42.127 | 1.00 | 17.56 | C |
| ATOM | 3570 | N | ALA | C | 109 | 25.040 | 62.066 | 42.354 | 1.00 | 16.53 | C |
| ATOM | 3571 | CA | ALA | C | 109 | 25.480 | 63.407 | 42.731 | 1.00 | 18.96 | C |
| ATOM | 3572 | CB | ALA | C | 109 | 26.857 | 63.340 | 43.389 | 1.00 | 17.50 | C |
| ATOM | 3573 | C | ALA | C | 109 | 25.515 | 64.386 | 41.566 | 1.00 | 21.48 | C |
| ATOM | 3574 | O | ALA | C | 109 | 25.302 | 65.583 | 41.750 | 1.00 | 20.60 | C |
| ATOM | 3575 | N | ASP | C | 110 | 25.793 | 63.872 | 40.372 | 1.00 | 24.47 | C |
| ATOM | 3576 | CA | ASP | C | 110 | 25.870 | 64.697 | 39.172 | 1.00 | 27.07 | C |
| ATOM | 3577 | CB | ASP | C | 110 | 24.464 | 65.106 | 38.723 | 1.00 | 29.92 | C |
| ATOM | 3578 | CG | ASP | C | 110 | 24.420 | 65.550 | 37.270 | 1.00 | 32.85 | C |
| ATOM | 3579 | OD1 | ASP | C | 110 | 25.135 | 66.511 | 36.912 | 1.00 | 34.29 | C |
| ATOM | 3580 | OD2 | ASP | C | 110 | 23.669 | 64.932 | 36.486 | 1.00 | 35.24 | C |
| ATOM | 3581 | C | ASP | C | 110 | 26.713 | 65.942 | 39.440 | 1.00 | 27.04 | C |
| ATOM | 3582 | O | ASP | C | 110 | 26.307 | 67.061 | 39.126 | 1.00 | 28.13 | C |
| ATOM | 3583 | N | VAL | C | 111 | 27.890 | 65.742 | 40.025 | 1.00 | 26.59 | C |
| ATOM | 3584 | CA | VAL | C | 111 | 28.781 | 66.853 | 40.336 | 1.00 | 26.68 | C |
| ATOM | 3585 | CB | VAL | C | 111 | 29.971 | 66.387 | 41.202 | 1.00 | 27.88 | C |
| ATOM | 3586 | CG1 | VAL | C | 111 | 29.455 | 65.744 | 42.486 | 1.00 | 26.40 | C |
| ATOM | 3587 | CG2 | VAL | C | 111 | 30.838 | 65.409 | 40.421 | 1.00 | 26.79 | C |
| ATOM | 3588 | C | VAL | C | 111 | 29.318 | 67.511 | 39.064 | 1.00 | 27.54 | C |
| ATOM | 3589 | O | VAL | C | 111 | 29.336 | 66.899 | 37.997 | 1.00 | 24.91 | C |
| ATOM | 3590 | N | LYS | C | 112 | 29.754 | 68.761 | 39.190 | 1.00 | 28.06 | C |
| ATOM | 3591 | CA | LYS | C | 112 | 30.283 | 69.513 | 38.058 | 1.00 | 29.54 | C |
| ATOM | 3592 | CB | LYS | C | 112 | 30.468 | 70.984 | 38.441 | 1.00 | 31.43 | C |
| ATOM | 3593 | CG | LYS | C | 112 | 29.188 | 71.806 | 38.418 | 1.00 | 33.66 | C |
| ATOM | 3594 | CD | LYS | C | 112 | 28.639 | 71.926 | 37.001 | 1.00 | 35.69 | C |
| ATOM | 3595 | CE | LYS | C | 112 | 27.423 | 72.837 | 36.950 | 1.00 | 36.22 | C |
| ATOM | 3596 | NZ | LYS | C | 112 | 26.883 | 72.964 | 35.567 | 1.00 | 37.24 | C |
| ATOM | 3597 | C | LYS | C | 112 | 31.601 | 68.967 | 37.521 | 1.00 | 29.27 | C |
| ATOM | 3598 | O | LYS | C | 112 | 31.738 | 68.735 | 36.321 | 1.00 | 28.83 | C |
| ATOM | 3599 | N | GLU | C | 113 | 32.571 | 68.776 | 38.410 | 1.00 | 28.93 | C |
| ATOM | 3600 | CA | GLU | C | 113 | 33.881 | 68.271 | 38.015 | 1.00 | 29.21 | C |
| ATOM | 3601 | CB | GLU | C | 113 | 34.986 | 69.234 | 38.468 | 1.00 | 32.90 | C |
| ATOM | 3602 | CG | GLU | C | 113 | 35.435 | 70.250 | 37.426 | 1.00 | 36.30 | C |
| ATOM | 3603 | CD | GLU | C | 113 | 34.383 | 71.292 | 37.115 | 1.00 | 38.79 | C |
| ATOM | 3604 | OE1 | GLU | C | 113 | 33.307 | 70.922 | 36.599 | 1.00 | 40.97 | C |
| ATOM | 3605 | OE2 | GLU | C | 113 | 34.635 | 72.486 | 37.387 | 1.00 | 40.46 | C |
| ATOM | 3606 | C | GLU | C | 113 | 34.166 | 66.891 | 38.592 | 1.00 | 27.87 | C |
| ATOM | 3607 | O | GLU | C | 113 | 34.921 | 66.756 | 39.551 | 1.00 | 27.04 | C |
| ATOM | 3608 | N | PRO | C | 114 | 33.571 | 65.844 | 38.007 | 1.00 | 25.95 | C |
| ATOM | 3609 | CD | PRO | C | 114 | 32.660 | 65.810 | 36.850 | 1.00 | 25.93 | C |
| ATOM | 3610 | CA | PRO | C | 114 | 33.814 | 64.496 | 38.525 | 1.00 | 25.41 | C |
| ATOM | 3611 | CB | PRO | C | 114 | 32.884 | 63.627 | 37.678 | 1.00 | 24.62 | C |
| ATOM | 3612 | CG | PRO | C | 114 | 32.799 | 64.383 | 36.385 | 1.00 | 26.29 | C |
| ATOM | 3613 | C | PRO | C | 114 | 35.276 | 64.058 | 38.443 | 1.00 | 24.88 | C |
| ATOM | 3614 | O | PRO | C | 114 | 35.703 | 63.176 | 39.189 | 1.00 | 23.14 | C |
| ATOM | 3615 | N | GLU | C | 115 | 36.047 | 64.679 | 37.554 | 1.00 | 24.67 | C |
| ATOM | 3616 | CA | GLU | C | 115 | 37.457 | 64.315 | 37.402 | 1.00 | 25.90 | C |
| ATOM | 3617 | CB | GLU | C | 115 | 38.050 | 64.934 | 36.134 | 1.00 | 26.95 | C |
| ATOM | 3618 | CG | GLU | C | 115 | 37.033 | 65.405 | 35.118 | 1.00 | 32.21 | C |
| ATOM | 3619 | CD | GLU | C | 115 | 36.446 | 66.759 | 35.468 | 1.00 | 32.66 | C |
| ATOM | 3620 | OE1 | GLU | C | 115 | 37.222 | 67.729 | 35.596 | 1.00 | 34.70 | C |
| ATOM | 3621 | OE2 | GLU | C | 115 | 35.209 | 66.854 | 35.607 | 1.00 | 35.20 | C |
| ATOM | 3622 | C | GLU | C | 115 | 38.307 | 64.745 | 38.591 | 1.00 | 25.10 | C |
| ATOM | 3623 | O | GLU | C | 115 | 39.403 | 64.226 | 38.798 | 1.00 | 26.06 | C |
| ATOM | 3624 | N | SER | C | 116 | 37.808 | 65.696 | 39.372 | 1.00 | 24.18 | C |
| ATOM | 3625 | CA | SER | C | 116 | 38.560 | 66.187 | 40.519 | 1.00 | 23.46 | C |
| ATOM | 3626 | CB | SER | C | 116 | 39.073 | 67.597 | 40.229 | 1.00 | 25.47 | C |
| ATOM | 3627 | OG | SER | C | 116 | 38.001 | 68.451 | 39.859 | 1.00 | 27.86 | C |
| ATOM | 3628 | C | SER | C | 116 | 37.753 | 66.206 | 41.808 | 1.00 | 21.97 | C |
| ATOM | 3629 | O | SER | C | 116 | 38.190 | 66.781 | 42.805 | 1.00 | 21.03 | C |
| ATOM | 3630 | N | PHE | C | 117 | 36.582 | 65.580 | 41.801 | 1.00 | 18.76 | C |
| ATOM | 3631 | CA | PHE | C | 117 | 35.762 | 65.581 | 43.002 | 1.00 | 17.59 | C |
| ATOM | 3632 | CB | PHE | C | 117 | 34.435 | 64.869 | 42.763 | 1.00 | 16.09 | C |
| ATOM | 3633 | CG | PHE | C | 117 | 33.469 | 65.044 | 43.890 | 1.00 | 15.95 | C |
| ATOM | 3634 | CD1 | PHE | C | 117 | 33.500 | 64.192 | 44.985 | 1.00 | 14.96 | C |
| ATOM | 3635 | CD2 | PHE | C | 117 | 32.584 | 66.114 | 43.895 | 1.00 | 15.75 | C |
| ATOM | 3636 | CE1 | PHE | C | 117 | 32.663 | 64.408 | 46.074 | 1.00 | 14.43 | C |
| ATOM | 3637 | CE2 | PHE | C | 117 | 31.744 | 66.339 | 44.980 | 1.00 | 15.84 | C |
| ATOM | 3638 | CZ | PHE | C | 117 | 31.785 | 65.484 | 46.070 | 1.00 | 13.92 | C |
| ATOM | 3639 | C | PHE | C | 117 | 36.513 | 64.934 | 44.160 | 1.00 | 16.90 | C |
| ATOM | 3640 | O | PHE | C | 117 | 37.003 | 63.809 | 44.051 | 1.00 | 17.86 | C |
| ATOM | 3641 | N | PRO | C | 118 | 36.610 | 65.645 | 45.291 | 1.00 | 15.79 | C |
| ATOM | 3642 | CD | PRO | C | 118 | 36.176 | 67.037 | 45.512 | 1.00 | 15.05 | C |
| ATOM | 3643 | CA | PRO | C | 118 | 37.318 | 65.129 | 46.462 | 1.00 | 14.57 | C |
| ATOM | 3644 | CB | PRO | C | 118 | 37.616 | 66.392 | 47.261 | 1.00 | 15.16 | C |
| ATOM | 3645 | CG | PRO | C | 118 | 36.423 | 67.232 | 46.993 | 1.00 | 14.73 | C |
| ATOM | 3646 | C | PRO | C | 118 | 36.625 | 64.070 | 47.305 | 1.00 | 14.64 | C |
| ATOM | 3647 | O | PRO | C | 118 | 35.504 | 64.256 | 47.783 | 1.00 | 13.53 | C |
| ATOM | 3648 | N | PHE | C | 119 | 37.321 | 62.954 | 47.470 | 1.00 | 12.45 | C |
| ATOM | 3649 | CA | PHE | C | 119 | 36.862 | 61.848 | 48.294 | 1.00 | 12.14 | C |
| ATOM | 3650 | CB | PHE | C | 119 | 36.835 | 60.540 | 47.499 | 1.00 | 14.09 | C |
| ATOM | 3651 | CG | PHE | C | 119 | 35.561 | 60.305 | 46.746 | 1.00 | 15.05 | C |
| ATOM | 3652 | CD1 | PHE | C | 119 | 34.463 | 59.735 | 47.376 | 1.00 | 16.53 | C |
| ATOM | 3653 | CD2 | PHE | C | 119 | 35.462 | 60.645 | 45.402 | 1.00 | 16.62 | C |
| ATOM | 3654 | CE1 | PHE | C | 119 | 33.276 | 59.502 | 46.677 | 1.00 | 18.80 | C |
| ATOM | 3655 | CE2 | PHE | C | 119 | 34.280 | 60.418 | 44.695 | 1.00 | 18.09 | C |
| ATOM | 3656 | CZ | PHE | C | 119 | 33.188 | 59.845 | 45.337 | 1.00 | 16.33 | C |
| ATOM | 3657 | C | PHE | C | 119 | 37.925 | 61.723 | 49.373 | 1.00 | 10.93 | C |
| ATOM | 3658 | O | PHE | C | 119 | 39.107 | 61.942 | 49.111 | 1.00 | 11.40 | C |
| ATOM | 3659 | N | VAL | C | 120 | 37.507 | 61.406 | 50.589 | 1.00 | 9.28 | C |
| ATOM | 3660 | CA | VAL | C | 120 | 38.448 | 61.191 | 51.680 | 1.00 | 9.48 | C |
| ATOM | 3661 | CB | VAL | C | 120 | 38.374 | 62.294 | 52.763 | 1.00 | 8.23 | C |
| ATOM | 3662 | CG1 | VAL | C | 120 | 39.245 | 61.903 | 53.958 | 1.00 | 7.90 | C |
| ATOM | 3663 | CG2 | VAL | C | 120 | 38.851 | 63.623 | 52.189 | 1.00 | 8.02 | C |
| ATOM | 3664 | C | VAL | C | 120 | 37.997 | 59.851 | 52.246 | 1.00 | 9.70 | C |
| ATOM | 3665 | O | VAL | C | 120 | 36.837 | 59.692 | 52.643 | 1.00 | 10.50 | C |
| ATOM | 3666 | N | ILE | C | 121 | 38.910 | 58.885 | 52.250 | 1.00 | 10.59 | C |
| ATOM | 3667 | CA | ILE | C | 121 | 38.607 | 57.537 | 52.713 | 1.00 | 9.78 | C |
| ATOM | 3668 | CB | ILE | C | 121 | 39.361 | 56.490 | 51.864 | 1.00 | 10.65 | C |
| ATOM | 3669 | CG2 | ILE | C | 121 | 38.812 | 55.101 | 52.133 | 1.00 | 10.97 | C |
| ATOM | 3670 | CG1 | ILE | C | 121 | 39.236 | 56.838 | 50.375 | 1.00 | 14.56 | C |
| ATOM | 3671 | CD1 | ILE | C | 121 | 37.805 | 57.043 | 49.898 | 1.00 | 14.34 | C |
| ATOM | 3672 | C | ILE | C | 121 | 38.969 | 57.325 | 54.168 | 1.00 | 8.72 | C |
| ATOM | 3673 | O | ILE | C | 121 | 40.074 | 57.665 | 54.601 | 1.00 | 7.91 | C |
| ATOM | 3674 | N | LEU | C | 122 | 38.034 | 56.756 | 54.923 | 1.00 | 7.94 | C |
| ATOM | 3675 | CA | LEU | C | 122 | 38.264 | 56.493 | 56.335 | 1.00 | 8.77 | C |
| ATOM | 3676 | CB | LEU | C | 122 | 37.283 | 57.288 | 57.193 | 1.00 | 9.27 | C |
| ATOM | 3677 | CG | LEU | C | 122 | 37.150 | 58.790 | 56.953 | 1.00 | 12.23 | C |
| ATOM | 3678 | CD1 | LEU | C | 122 | 36.336 | 59.387 | 58.095 | 1.00 | 17.58 | C |
| ATOM | 3679 | CD2 | LEU | C | 122 | 38.521 | 59.441 | 56.881 | 1.00 | 15.05 | C |
| ATOM | 3680 | C | LEU | C | 122 | 38.125 | 55.018 | 56.698 | 1.00 | 8.62 | C |
| ATOM | 3681 | O | LEU | C | 122 | 37.073 | 54.413 | 56.465 | 1.00 | 9.88 | C |
| ATOM | 3682 | N | GLY | C | 123 | 39.198 | 54.457 | 57.254 | 1.00 | 6.59 | C |
| ATOM | 3683 | CA | GLY | C | 123 | 39.203 | 53.078 | 57.720 | 1.00 | 5.82 | C |
| ATOM | 3684 | C | GLY | C | 123 | 38.962 | 53.210 | 59.213 | 1.00 | 5.53 | C |
| ATOM | 3685 | O | GLY | C | 123 | 39.877 | 53.496 | 59.987 | 1.00 | 7.11 | C |
| ATOM | 3686 | N | ASN | C | 124 | 37.716 | 53.000 | 59.622 | 1.00 | 5.65 | C |
| ATOM | 3687 | CA | ASN | C | 124 | 37.323 | 53.174 | 61.008 | 1.00 | 6.07 | C |
| ATOM | 3688 | CB | ASN | C | 124 | 35.898 | 53.735 | 61.037 | 1.00 | 7.18 | C |
| ATOM | 3689 | CG | ASN | C | 124 | 35.511 | 54.274 | 62.390 | 1.00 | 6.45 | C |
| ATOM | 3690 | OD1 | ASN | C | 124 | 36.271 | 55.010 | 63.012 | 1.00 | 5.46 | C |
| ATOM | 3691 | ND2 | ASN | C | 124 | 34.311 | 53.924 | 62.850 | 1.00 | 6.62 | C |
| ATOM | 3692 | C | ASN | C | 124 | 37.426 | 51.943 | 61.899 | 1.00 | 5.95 | C |
| ATOM | 3693 | O | ASN | C | 124 | 37.604 | 50.822 | 61.422 | 1.00 | 7.04 | C |
| ATOM | 3694 | N | LYS | C | 125 | 37.307 | 52.182 | 63.201 | 1.00 | 7.80 | C |
| ATOM | 3695 | CA | LYS | C | 125 | 37.369 | 51.146 | 64.227 | 1.00 | 7.51 | C |
| ATOM | 3696 | CB | LYS | C | 125 | 36.342 | 50.041 | 63.936 | 1.00 | 8.05 | C |
| ATOM | 3697 | CG | LYS | C | 125 | 34.898 | 50.525 | 63.840 | 1.00 | 8.66 | C |
| ATOM | 3698 | CD | LYS | C | 125 | 33.907 | 49.355 | 63.907 | 1.00 | 9.43 | C |
| ATOM | 3699 | CE | LYS | C | 125 | 32.459 | 49.817 | 63.745 | 1.00 | 9.67 | C |
| ATOM | 3700 | NZ | LYS | C | 125 | 31.494 | 48.665 | 63.791 | 1.00 | 7.86 | C |
| ATOM | 3701 | C | LYS | C | 125 | 38.758 | 50.526 | 64.392 | 1.00 | 8.41 | C |
| ATOM | 3702 | O | LYS | C | 125 | 38.879 | 49.349 | 64.719 | 1.00 | 7.38 | C |
| ATOM | 3703 | N | ILE | C | 126 | 39.804 | 51.323 | 64.195 | 1.00 | 7.28 | C |
| ATOM | 3704 | CA | ILE | C | 126 | 41.167 | 50.817 | 64.325 | 1.00 | 9.24 | C |
| ATOM | 3705 | CB | ILE | C | 126 | 42.193 | 51.877 | 63.898 | 1.00 | 10.67 | C |
| ATOM | 3706 | CG2 | ILE | C | 126 | 42.040 | 52.171 | 62.411 | 1.00 | 14.03 | C |
| ATOM | 3707 | CG1 | ILE | C | 126 | 42.003 | 53.144 | 64.735 | 1.00 | 10.81 | C |
| ATOM | 3708 | CD1 | ILE | C | 126 | 42.971 | 54.255 | 64.407 | 1.00 | 17.44 | C |
| ATOM | 3709 | C | ILE | C | 126 | 41.506 | 50.362 | 65.742 | 1.00 | 9.45 | C |
| ATOM | 3710 | O | ILE | C | 126 | 42.510 | 49.684 | 65.955 | 1.00 | 10.21 | C |
| ATOM | 3711 | N | ASP | C | 127 | 40.669 | 50.723 | 66.708 | 1.00 | 9.30 | C |
| ATOM | 3712 | CA | ASP | C | 127 | 40.916 | 50.342 | 68.093 | 1.00 | 9.35 | C |
| ATOM | 3713 | CB | ASP | C | 127 | 40.185 | 51.293 | 69.045 | 1.00 | 10.10 | C |
| ATOM | 3714 | CG | ASP | C | 127 | 38.693 | 51.338 | 68.792 | 1.00 | 8.28 | C |
| ATOM | 3715 | OD1 | ASP | C | 127 | 38.277 | 51.929 | 67.769 | 1.00 | 9.96 | C |
| ATOM | 3716 | OD2 | ASP | C | 127 | 37.936 | 50.773 | 69.608 | 1.00 | 9.91 | C |
| ATOM | 3717 | C | ASP | C | 127 | 40.506 | 48.897 | 68.391 | 1.00 | 8.66 | C |
| ATOM | 3718 | O | ASP | C | 127 | 40.867 | 48.348 | 69.434 | 1.00 | 11.14 | C |
| ATOM | 3719 | N | ILE | C | 128 | 39.749 | 48.284 | 67.486 | 1.00 | 8.28 | C |
| ATOM | 3720 | CA | ILE | C | 128 | 39.325 | 46.900 | 67.681 | 1.00 | 8.44 | C |
| ATOM | 3721 | CB | ILE | C | 128 | 38.034 | 46.590 | 66.888 | 1.00 | 7.83 | C |
| ATOM | 3722 | CG2 | ILE | C | 128 | 37.710 | 45.101 | 66.963 | 1.00 | 7.20 | C |
| ATOM | 3723 | CG1 | ILE | C | 128 | 36.875 | 47.414 | 67.454 | 1.00 | 9.15 | C |
| ATOM | 3724 | CD1 | ILE | C | 128 | 35.561 | 47.209 | 66.714 | 1.00 | 9.77 | C |
| ATOM | 3725 | C | ILE | C | 128 | 40.441 | 45.961 | 67.230 | 1.00 | 9.75 | C |
| ATOM | 3726 | O | ILE | C | 128 | 41.018 | 46.139 | 66.164 | 1.00 | 9.90 | C |
| ATOM | 3727 | N | SER | C | 129 | 40.747 | 44.963 | 68.052 | 1.00 | 10.55 | C |
| ATOM | 3728 | CA | SER | C | 129 | 41.808 | 44.018 | 67.728 | 1.00 | 11.73 | C |
| ATOM | 3729 | CB | SER | C | 129 | 42.103 | 43.132 | 68.941 | 1.00 | 14.64 | C |
| ATOM | 3730 | OG | SER | C | 129 | 41.021 | 42.243 | 69.176 | 1.00 | 20.77 | C |
| ATOM | 3731 | C | SER | C | 129 | 41.476 | 43.130 | 66.528 | 1.00 | 10.15 | C |
| ATOM | 3732 | O | SER | C | 129 | 40.308 | 42.940 | 66.175 | 1.00 | 11.21 | C |
| ATOM | 3733 | N | GLU | C | 130 | 42.527 | 42.596 | 65.913 | 1.00 | 8.79 | C |
| ATOM | 3734 | CA | GLU | C | 130 | 42.433 | 41.703 | 64.760 | 1.00 | 9.32 | C |
| ATOM | 3735 | CB | GLU | C | 130 | 41.659 | 40.430 | 65.132 | 1.00 | 11.28 | C |
| ATOM | 3736 | CG | GLU | C | 130 | 41.898 | 39.908 | 66.549 | 1.00 | 13.21 | C |
| ATOM | 3737 | CD | GLU | C | 130 | 43.329 | 39.486 | 66.833 | 1.00 | 12.72 | C |
| ATOM | 3738 | OE1 | GLU | C | 130 | 44.194 | 39.567 | 65.931 | 1.00 | 11.74 | C |
| ATOM | 3739 | OE2 | GLU | C | 130 | 43.587 | 39.061 | 67.981 | 1.00 | 12.07 | C |
| ATOM | 3740 | C | GLU | C | 130 | 41.801 | 42.327 | 63.513 | 1.00 | 8.80 | C |
| ATOM | 3741 | O | GLU | C | 130 | 40.783 | 41.837 | 63.017 | 1.00 | 10.16 | C |
| ATOM | 3742 | N | ARG | C | 131 | 42.415 | 43.388 | 62.995 | 1.00 | 10.10 | C |
| ATOM | 3743 | CA | ARG | C | 131 | 41.908 | 44.068 | 61.797 | 1.00 | 12.44 | C |
| ATOM | 3744 | CB | ARG | C | 131 | 42.904 | 45.091 | 61.256 | 1.00 | 14.01 | C |
| ATOM | 3745 | CG | ARG | C | 131 | 43.672 | 45.899 | 62.250 | 1.00 | 17.16 | C |
| ATOM | 3746 | CD | ARG | C | 131 | 44.766 | 46.660 | 61.514 | 1.00 | 14.61 | C |
| ATOM | 3747 | NE | ARG | C | 131 | 44.298 | 47.186 | 60.228 | 1.00 | 14.47 | C |
| ATOM | 3748 | CZ | ARG | C | 131 | 44.623 | 48.385 | 59.752 | 1.00 | 12.92 | C |
| ATOM | 3749 | NH1 | ARG | C | 131 | 45.412 | 49.184 | 60.458 | 1.00 | 13.03 | C |
| ATOM | 3750 | NH2 | ARG | C | 131 | 44.172 | 48.785 | 58.567 | 1.00 | 9.43 | C |
| ATOM | 3751 | C | ARG | C | 131 | 41.745 | 43.046 | 60.691 | 1.00 | 12.19 | C |
| ATOM | 3752 | O | ARG | C | 131 | 42.537 | 42.112 | 60.598 | 1.00 | 14.00 | C |
| ATOM | 3753 | N | GLN | C | 132 | 40.757 | 43.252 | 59.826 | 1.00 | 11.66 | C |
| ATOM | 3754 | CA | GLN | C | 132 | 40.531 | 42.346 | 58.709 | 1.00 | 11.13 | C |
| ATOM | 3755 | CB | GLN | C | 132 | 39.069 | 41.907 | 58.673 | 1.00 | 13.70 | C |
| ATOM | 3756 | CG | GLN | C | 132 | 38.733 | 40.946 | 59.797 | 1.00 | 16.18 | C |
| ATOM | 3757 | CD | GLN | C | 132 | 39.634 | 39.726 | 59.786 | 1.00 | 17.42 | C |
| ATOM | 3758 | OE1 | GLN | C | 132 | 39.571 | 38.907 | 58.871 | 1.00 | 19.90 | C |
| ATOM | 3759 | NE2 | GLN | C | 132 | 40.490 | 39.607 | 60.798 | 1.00 | 15.96 | C |
| ATOM | 3760 | C | GLN | C | 132 | 40.940 | 42.968 | 57.376 | 1.00 | 11.19 | C |
| ATOM | 3761 | O | GLN | C | 132 | 41.007 | 42.279 | 56.354 | 1.00 | 11.50 | C |
| ATOM | 3762 | N | VAL | C | 133 | 41.218 | 44.270 | 57.396 | 1.00 | 9.39 | C |
| ATOM | 3763 | CA | VAL | C | 133 | 41.651 | 44.987 | 56.199 | 1.00 | 8.85 | C |
| ATOM | 3764 | CB | VAL | C | 133 | 40.667 | 46.133 | 55.833 | 1.00 | 7.03 | C |
| ATOM | 3765 | CG1 | VAL | C | 133 | 41.143 | 46.850 | 54.568 | 1.00 | 9.44 | C |
| ATOM | 3766 | CG2 | VAL | C | 133 | 39.265 | 45.575 | 55.633 | 1.00 | 8.24 | C |
| ATOM | 3767 | C | VAL | C | 133 | 43.019 | 45.578 | 56.527 | 1.00 | 7.38 | C |
| ATOM | 3768 | O | VAL | C | 133 | 43.149 | 46.372 | 57.460 | 1.00 | 6.70 | C |
| ATOM | 3769 | N | SER | C | 134 | 44.041 | 45.174 | 55.781 | 1.00 | 9.69 | C |
| ATOM | 3770 | CA | SER | C | 134 | 45.388 | 45.670 | 56.035 | 1.00 | 8.91 | C |
| ATOM | 3771 | CB | SER | C | 134 | 46.432 | 44.810 | 55.314 | 1.00 | 10.03 | C |
| ATOM | 3772 | OG | SER | C | 134 | 46.368 | 44.978 | 53.908 | 1.00 | 12.64 | C |
| ATOM | 3773 | C | SER | C | 134 | 45.508 | 47.113 | 55.579 | 1.00 | 8.74 | C |
| ATOM | 3774 | O | SER | C | 134 | 44.799 | 47.552 | 54.674 | 1.00 | 8.42 | C |
| ATOM | 3775 | N | THR | C | 135 | 46.406 | 47.854 | 56.212 | 1.00 | 9.20 | C |
| ATOM | 3776 | CA | THR | C | 135 | 46.583 | 49.245 | 55.851 | 1.00 | 8.96 | C |
| ATOM | 3777 | CB | THR | C | 135 | 47.494 | 49.971 | 56.868 | 1.00 | 10.05 | C |
| ATOM | 3778 | OG1 | THR | C | 135 | 47.395 | 51.383 | 56.662 | 1.00 | 7.32 | C |
| ATOM | 3779 | CG2 | THR | C | 135 | 48.946 | 49.532 | 56.715 | 1.00 | 8.78 | C |
| ATOM | 3780 | C | THR | C | 135 | 47.146 | 49.356 | 54.438 | 1.00 | 10.10 | C |
| ATOM | 3781 | O | THR | C | 135 | 46.813 | 50.287 | 53.703 | 1.00 | 10.30 | C |
| ATOM | 3782 | N | GLU | C | 136 | 47.979 | 48.395 | 54.044 | 1.00 | 11.44 | C |
| ATOM | 3783 | CA | GLU | C | 136 | 48.554 | 48.403 | 52.700 | 1.00 | 12.34 | C |
| ATOM | 3784 | CB | GLU | C | 136 | 49.550 | 47.247 | 52.526 | 1.00 | 14.03 | C |
| ATOM | 3785 | CG | GLU | C | 136 | 50.848 | 47.372 | 53.311 | 1.00 | 16.49 | C |
| ATOM | 3786 | CD | GLU | C | 136 | 50.676 | 47.116 | 54.795 | 1.00 | 17.93 | C |
| ATOM | 3787 | OE1 | GLU | C | 136 | 49.786 | 46.317 | 55.163 | 1.00 | 17.01 | C |
| ATOM | 3788 | OE2 | GLU | C | 136 | 51.446 | 47.700 | 55.589 | 1.00 | 18.60 | C |
| ATOM | 3789 | C | GLU | C | 136 | 47.466 | 48.278 | 51.631 | 1.00 | 11.70 | C |
| ATOM | 3790 | O | GLU | C | 136 | 47.431 | 49.047 | 50.670 | 1.00 | 10.43 | C |
| ATOM | 3791 | N | GLU | C | 137 | 46.577 | 47.305 | 51.809 | 1.00 | 11.74 | C |
| ATOM | 3792 | CA | GLU | C | 137 | 45.497 | 47.061 | 50.859 | 1.00 | 12.59 | C |
| ATOM | 3793 | CB | GLU | C | 137 | 44.721 | 45.809 | 51.277 | 1.00 | 16.05 | C |
| ATOM | 3794 | CG | GLU | C | 137 | 43.789 | 45.259 | 50.215 | 1.00 | 22.30 | C |
| ATOM | 3795 | CD | GLU | C | 137 | 43.107 | 43.973 | 50.654 | 1.00 | 26.09 | C |
| ATOM | 3796 | OE1 | GLU | C | 137 | 43.819 | 43.013 | 51.018 | 1.00 | 28.24 | C |
| ATOM | 3797 | OE2 | GLU | C | 137 | 41.860 | 43.919 | 50.631 | 1.00 | 30.06 | C |
| ATOM | 3798 | C | GLU | C | 137 | 44.548 | 48.253 | 50.761 | 1.00 | 10.70 | C |
| ATOM | 3799 | O | GLU | C | 137 | 44.072 | 48.601 | 49.677 | 1.00 | 8.35 | C |
| ATOM | 3800 | N | ALA | C | 138 | 44.273 | 48.882 | 51.897 | 1.00 | 8.89 | C |
| ATOM | 3801 | CA | ALA | C | 138 | 43.385 | 50.033 | 51.917 | 1.00 | 10.51 | C |
| ATOM | 3802 | CB | ALA | C | 138 | 43.074 | 50.419 | 53.355 | 1.00 | 10.54 | C |
| ATOM | 3803 | C | ALA | C | 138 | 44.025 | 51.209 | 51.178 | 1.00 | 9.02 | C |
| ATOM | 3804 | O | ALA | C | 138 | 43.385 | 51.855 | 50.347 | 1.00 | 9.72 | C |
| ATOM | 3805 | N | GLN | C | 139 | 45.290 | 51.478 | 51.484 | 1.00 | 8.53 | C |
| ATOM | 3806 | CA | GLN | C | 139 | 46.009 | 52.577 | 50.851 | 1.00 | 9.26 | C |
| ATOM | 3807 | CB | GLN | C | 139 | 47.390 | 52.750 | 51.494 | 1.00 | 9.10 | C |
| ATOM | 3808 | CG | GLN | C | 139 | 47.328 | 53.337 | 52.894 | 1.00 | 8.64 | C |
| ATOM | 3809 | CD | GLN | C | 139 | 48.698 | 53.565 | 53.493 | 1.00 | 10.86 | C |
| ATOM | 3810 | OE1 | GLN | C | 139 | 49.517 | 54.292 | 52.933 | 1.00 | 9.66 | C |
| ATOM | 3811 | NE2 | GLN | C | 139 | 48.956 | 52.945 | 54.646 | 1.00 | 8.87 | C |
| ATOM | 3812 | C | GLN | C | 139 | 46.149 | 52.350 | 49.355 | 1.00 | 9.63 | C |
| ATOM | 3813 | O | GLN | C | 139 | 46.079 | 53.296 | 48.571 | 1.00 | 11.89 | C |
| ATOM | 3814 | N | ALA | C | 140 | 46.344 | 51.095 | 48.960 | 1.00 | 10.47 | C |
| ATOM | 3815 | CA | ALA | C | 140 | 46.476 | 50.757 | 47.551 | 1.00 | 11.48 | C |
| ATOM | 3816 | CB | ALA | C | 140 | 46.774 | 49.270 | 47.392 | 1.00 | 12.78 | C |
| ATOM | 3817 | C | ALA | C | 140 | 45.194 | 51.123 | 46.806 | 1.00 | 12.84 | C |
| ATOM | 3818 | O | ALA | C | 140 | 45.240 | 51.612 | 45.676 | 1.00 | 12.78 | C |
| ATOM | 3819 | N | TRP | C | 141 | 44.046 | 50.897 | 47.438 | 1.00 | 12.80 | C |
| ATOM | 3820 | CA | TRP | C | 141 | 42.777 | 51.227 | 46.800 | 1.00 | 13.20 | C |
| ATOM | 3821 | CB | TRP | C | 141 | 41.593 | 50.739 | 47.646 | 1.00 | 13.22 | C |
| ATOM | 3822 | CG | TRP | C | 141 | 40.269 | 50.914 | 46.951 | 1.00 | 12.07 | C |
| ATOM | 3823 | CD2 | TRP | C | 141 | 39.444 | 52.084 | 46.959 | 1.00 | 13.20 | C |
| ATOM | 3824 | CE2 | TRP | C | 141 | 38.345 | 51.828 | 46.106 | 1.00 | 13.94 | C |
| ATOM | 3825 | CE3 | TRP | C | 141 | 39.526 | 53.327 | 47.600 | 1.00 | 10.68 | C |
| ATOM | 3826 | CD1 | TRP | C | 141 | 39.652 | 50.018 | 46.120 | 1.00 | 13.61 | C |
| ATOM | 3827 | NE1 | TRP | C | 141 | 38.499 | 50.560 | 45.609 | 1.00 | 12.86 | C |
| ATOM | 3828 | CZ2 | TRP | C | 141 | 37.336 | 52.772 | 45.878 | 1.00 | 14.20 | C |
| ATOM | 3829 | CZ3 | TRP | C | 141 | 38.524 | 54.266 | 47.373 | 1.00 | 13.65 | C |
| ATOM | 3830 | CH2 | TRP | C | 141 | 37.443 | 53.980 | 46.518 | 1.00 | 13.29 | C |
| ATOM | 3831 | C | TRP | C | 141 | 42.674 | 52.737 | 46.608 | 1.00 | 13.48 | C |
| ATOM | 3832 | O | TRP | C | 141 | 42.240 | 53.214 | 45.555 | 1.00 | 12.69 | C |
| ATOM | 3833 | N | CYS | C | 142 | 43.073 | 53.493 | 47.629 | 1.00 | 13.23 | C |
| ATOM | 3834 | CA | CYS | C | 142 | 43.012 | 54.945 | 47.557 | 1.00 | 14.02 | C |
| ATOM | 3835 | CB | CYS | C | 142 | 43.405 | 55.554 | 48.904 | 1.00 | 13.84 | C |
| ATOM | 3836 | SG | CYS | C | 142 | 42.288 | 55.081 | 50.243 | 1.00 | 12.97 | C |
| ATOM | 3837 | C | CYS | C | 142 | 43.913 | 55.484 | 46.451 | 1.00 | 15.18 | C |
| ATOM | 3838 | O | CYS | C | 142 | 43.553 | 56.426 | 45.748 | 1.00 | 15.17 | C |
| ATOM | 3839 | N | ARG | C | 143 | 45.085 | 54.881 | 46.293 | 1.00 | 15.18 | C |
| ATOM | 3840 | CA | ARG | C | 143 | 46.013 | 55.315 | 45.259 | 1.00 | 17.44 | C |
| ATOM | 3841 | CB | ARG | C | 143 | 47.392 | 54.681 | 45.487 | 1.00 | 18.61 | C |
| ATOM | 3842 | CG | ARG | C | 143 | 48.369 | 55.589 | 46.212 | 1.00 | 22.81 | C |
| ATOM | 3843 | CD | ARG | C | 143 | 48.975 | 56.616 | 45.262 | 1.00 | 22.21 | C |
| ATOM | 3844 | NE | ARG | C | 143 | 50.244 | 56.158 | 44.705 | 1.00 | 26.91 | C |
| ATOM | 3845 | CZ | ARG | C | 143 | 50.928 | 56.801 | 43.764 | 1.00 | 28.39 | C |
| ATOM | 3846 | NH1 | ARG | C | 143 | 50.463 | 57.937 | 43.263 | 1.00 | 30.51 | C |
| ATOM | 3847 | NH2 | ARG | C | 143 | 52.086 | 56.316 | 43.332 | 1.00 | 29.85 | C |
| ATOM | 3848 | C | ARG | C | 143 | 45.514 | 54.965 | 43.861 | 1.00 | 17.93 | C |
| ATOM | 3849 | O | ARG | C | 143 | 45.668 | 55.754 | 42.929 | 1.00 | 18.72 | C |
| ATOM | 3850 | N | ASP | C | 144 | 44.899 | 53.795 | 43.724 | 1.00 | 16.62 | C |
| ATOM | 3851 | CA | ASP | C | 144 | 44.411 | 53.336 | 42.429 | 1.00 | 19.50 | C |
| ATOM | 3852 | CB | ASP | C | 144 | 44.371 | 51.807 | 42.393 | 1.00 | 22.26 | C |
| ATOM | 3853 | CG | ASP | C | 144 | 45.714 | 51.180 | 42.692 | 1.00 | 26.08 | C |
| ATOM | 3854 | OD1 | ASP | C | 144 | 46.732 | 51.652 | 42.141 | 1.00 | 27.93 | C |
| ATOM | 3855 | OD2 | ASP | C | 144 | 45.749 | 50.206 | 43.472 | 1.00 | 28.35 | C |
| ATOM | 3856 | C | ASP | C | 144 | 43.049 | 53.863 | 41.992 | 1.00 | 18.94 | C |
| ATOM | 3857 | O | ASP | C | 144 | 42.657 | 53.670 | 40.841 | 1.00 | 19.88 | C |
| ATOM | 3858 | N | ASN | C | 145 | 42.321 | 54.518 | 42.888 | 1.00 | 18.22 | C |
| ATOM | 3859 | CA | ASN | C | 145 | 41.006 | 55.028 | 42.515 | 1.00 | 17.77 | C |
| ATOM | 3860 | CB | ASN | C | 145 | 39.923 | 54.394 | 43.391 | 1.00 | 19.59 | C |
| ATOM | 3861 | CG | ASN | C | 145 | 39.763 | 52.913 | 43.128 | 1.00 | 20.12 | C |
| ATOM | 3862 | OD1 | ASN | C | 145 | 40.588 | 52.102 | 43.545 | 1.00 | 21.63 | C |
| ATOM | 3863 | ND2 | ASN | C | 145 | 38.706 | 52.552 | 42.414 | 1.00 | 21.65 | C |
| ATOM | 3864 | C | ASN | C | 145 | 40.865 | 56.541 | 42.544 | 1.00 | 17.84 | C |
| ATOM | 3865 | O | ASN | C | 145 | 39.757 | 57.058 | 42.656 | 1.00 | 18.44 | C |
| ATOM | 3866 | N | GLY | C | 146 | 41.981 | 57.251 | 42.432 | 1.00 | 17.16 | C |
| ATOM | 3867 | CA | GLY | C | 146 | 41.918 | 58.702 | 42.436 | 1.00 | 17.00 | C |
| ATOM | 3868 | C | GLY | C | 146 | 42.942 | 59.373 | 43.327 | 1.00 | 17.36 | C |
| ATOM | 3869 | O | GLY | C | 146 | 42.962 | 60.602 | 43.438 | 1.00 | 16.22 | C |
| ATOM | 3870 | N | ASP | C | 147 | 43.792 | 58.570 | 43.963 | 1.00 | 18.12 | C |
| ATOM | 3871 | CA | ASP | C | 147 | 44.823 | 59.087 | 44.853 | 1.00 | 18.75 | C |
| ATOM | 3872 | CB | ASP | C | 147 | 45.817 | 59.946 | 44.065 | 1.00 | 25.39 | C |
| ATOM | 3873 | CG | ASP | C | 147 | 47.010 | 60.369 | 44.900 | 1.00 | 29.42 | C |
| ATOM | 3874 | OD1 | ASP | C | 147 | 47.707 | 59.479 | 45.435 | 1.00 | 30.91 | C |
| ATOM | 3875 | OD2 | ASP | C | 147 | 47.252 | 61.591 | 45.022 | 1.00 | 34.28 | C |
| ATOM | 3876 | C | ASP | C | 147 | 44.195 | 59.911 | 45.976 | 1.00 | 17.44 | C |
| ATOM | 3877 | O | ASP | C | 147 | 44.580 | 61.059 | 46.215 | 1.00 | 17.13 | C |
| ATOM | 3878 | N | TYR | C | 148 | 43.228 | 59.319 | 46.670 | 1.00 | 14.54 | C |
| ATOM | 3879 | CA | TYR | C | 148 | 42.547 | 60.009 | 47.761 | 1.00 | 13.15 | C |
| ATOM | 3880 | CB | TYR | C | 148 | 41.151 | 59.431 | 47.994 | 1.00 | 13.69 | C |
| ATOM | 3881 | CG | TYR | C | 148 | 40.267 | 59.346 | 46.776 | 1.00 | 14.31 | C |
| ATOM | 3882 | CD1 | TYR | C | 148 | 40.023 | 60.467 | 45.981 | 1.00 | 14.44 | C |
| ATOM | 3883 | CE1 | TYR | C | 148 | 39.161 | 60.398 | 44.891 | 1.00 | 16.92 | C |
| ATOM | 3884 | CD2 | TYR | C | 148 | 39.629 | 58.153 | 46.446 | 1.00 | 13.73 | C |
| ATOM | 3885 | CE2 | TYR | C | 148 | 38.769 | 58.074 | 45.366 | 1.00 | 15.52 | C |
| ATOM | 3886 | CZ | TYR | C | 148 | 38.536 | 59.198 | 44.594 | 1.00 | 16.00 | C |
| ATOM | 3887 | OH | TYR | C | 148 | 37.660 | 59.117 | 43.540 | 1.00 | 18.47 | C |
| ATOM | 3888 | C | TYR | C | 148 | 43.288 | 59.914 | 49.084 | 1.00 | 11.56 | C |
| ATOM | 3889 | O | TYR | C | 148 | 44.050 | 58.974 | 49.321 | 1.00 | 11.24 | C |
| ATOM | 3890 | N | PRO | C | 149 | 43.070 | 60.897 | 49.967 | 1.00 | 11.67 | C |
| ATOM | 3891 | CD | PRO | C | 149 | 42.399 | 62.185 | 49.721 | 1.00 | 12.23 | C |
| ATOM | 3892 | CA | PRO | C | 149 | 43.720 | 60.890 | 51.278 | 1.00 | 10.52 | C |
| ATOM | 3893 | CB | PRO | C | 149 | 43.338 | 62.247 | 51.861 | 1.00 | 11.72 | C |
| ATOM | 3894 | CG | PRO | C | 149 | 43.161 | 63.111 | 50.632 | 1.00 | 12.97 | C |
| ATOM | 3895 | C | PRO | C | 149 | 43.069 | 59.741 | 52.050 | 1.00 | 10.39 | C |
| ATOM | 3896 | O | PRO | C | 149 | 41.869 | 59.499 | 51.902 | 1.00 | 10.30 | C |
| ATOM | 3897 | N | TYR | C | 150 | 43.855 | 59.039 | 52.859 | 1.00 | 9.39 | C |
| ATOM | 3898 | CA | TYR | C | 150 | 43.353 | 57.914 | 53.643 | 1.00 | 9.87 | C |
| ATOM | 3899 | CB | TYR | C | 150 | 43.990 | 56.612 | 53.147 | 1.00 | 8.52 | C |
| ATOM | 3900 | CG | TYR | C | 150 | 43.670 | 55.411 | 54.001 | 1.00 | 8.25 | C |
| ATOM | 3901 | CD1 | TYR | C | 150 | 42.357 | 54.964 | 54.136 | 1.00 | 9.28 | C |
| ATOM | 3902 | CE1 | TYR | C | 150 | 42.048 | 53.868 | 54.936 | 1.00 | 9.14 | C |
| ATOM | 3903 | CD2 | TYR | C | 150 | 44.677 | 54.729 | 54.685 | 1.00 | 8.46 | C |
| ATOM | 3904 | CE2 | TYR | C | 150 | 44.382 | 53.626 | 55.486 | 1.00 | 8.48 | C |
| ATOM | 3905 | CZ | TYR | C | 150 | 43.063 | 53.204 | 55.606 | 1.00 | 8.91 | C |
| ATOM | 3906 | OH | TYR | C | 150 | 42.751 | 52.129 | 56.400 | 1.00 | 9.56 | C |
| ATOM | 3907 | C | TYR | C | 150 | 43.661 | 58.097 | 55.125 | 1.00 | 9.56 | C |
| ATOM | 3908 | O | TYR | C | 150 | 44.787 | 58.427 | 55.494 | 1.00 | 11.31 | C |
| ATOM | 3909 | N | PHE | C | 151 | 42.661 | 57.879 | 55.975 | 1.00 | 8.09 | C |
| ATOM | 3910 | CA | PHE | C | 151 | 42.846 | 58.024 | 57.416 | 1.00 | 7.72 | C |
| ATOM | 3911 | CB | PHE | C | 151 | 42.107 | 59.256 | 57.955 | 1.00 | 10.51 | C |
| ATOM | 3912 | CG | PHE | C | 151 | 42.620 | 60.562 | 57.427 | 1.00 | 7.93 | C |
| ATOM | 3913 | CD1 | PHE | C | 151 | 42.188 | 61.048 | 56.199 | 1.00 | 12.24 | C |
| ATOM | 3914 | CD2 | PHE | C | 151 | 43.541 | 61.302 | 58.159 | 1.00 | 11.67 | C |
| ATOM | 3915 | CE1 | PHE | C | 151 | 42.667 | 62.263 | 55.704 | 1.00 | 11.66 | C |
| ATOM | 3916 | CE2 | PHE | C | 151 | 44.029 | 62.517 | 57.674 | 1.00 | 11.37 | C |
| ATOM | 3917 | CZ | PHE | C | 151 | 43.589 | 62.996 | 56.443 | 1.00 | 11.54 | C |
| ATOM | 3918 | C | PHE | C | 151 | 42.296 | 56.818 | 58.148 | 1.00 | 7.47 | C |
| ATOM | 3919 | O | PHE | C | 151 | 41.204 | 56.355 | 57.827 | 1.00 | 9.56 | C |
| ATOM | 3920 | N | GLU | C | 152 | 43.050 | 56.316 | 59.121 | 1.00 | 7.46 | C |
| ATOM | 3921 | CA | GLU | C | 152 | 42.596 | 55.196 | 59.940 | 1.00 | 8.35 | C |
| ATOM | 3922 | CB | GLU | C | 152 | 43.733 | 54.211 | 60.210 | 1.00 | 8.43 | C |
| ATOM | 3923 | CG | GLU | C | 152 | 44.356 | 53.681 | 58.923 | 1.00 | 13.46 | C |
| ATOM | 3924 | CD | GLU | C | 152 | 44.919 | 52.274 | 59.036 | 1.00 | 11.31 | C |
| ATOM | 3925 | OE1 | GLU | C | 152 | 45.532 | 51.945 | 60.071 | 1.00 | 12.04 | C |
| ATOM | 3926 | OE2 | GLU | C | 152 | 44.762 | 51.498 | 58.068 | 1.00 | 10.49 | C |
| ATOM | 3927 | C | GLU | C | 152 | 42.113 | 55.865 | 61.219 | 1.00 | 8.54 | C |
| ATOM | 3928 | O | GLU | C | 152 | 42.891 | 56.479 | 61.960 | 1.00 | 9.91 | C |
| ATOM | 3929 | N | THR | C | 153 | 40.815 | 55.754 | 61.459 | 1.00 | 6.99 | C |
| ATOM | 3930 | CA | THR | C | 153 | 40.181 | 56.412 | 62.583 | 1.00 | 8.72 | C |
| ATOM | 3931 | CB | THR | C | 153 | 39.063 | 57.347 | 62.077 | 1.00 | 7.01 | C |
| ATOM | 3932 | OG1 | THR | C | 153 | 38.025 | 56.557 | 61.479 | 1.00 | 8.39 | C |
| ATOM | 3933 | CG2 | THR | C | 153 | 39.604 | 58.305 | 61.012 | 1.00 | 8.76 | C |
| ATOM | 3934 | C | THR | C | 153 | 39.550 | 55.518 | 63.630 | 1.00 | 7.85 | C |
| ATOM | 3935 | O | THR | C | 153 | 39.417 | 54.305 | 63.458 | 1.00 | 7.27 | C |
| ATOM | 3936 | N | SER | C | 154 | 39.156 | 56.166 | 64.719 | 1.00 | 7.37 | C |
| ATOM | 3937 | CA | SER | C | 154 | 38.467 | 55.531 | 65.829 | 1.00 | 8.06 | C |
| ATOM | 3938 | CB | SER | C | 154 | 39.441 | 55.043 | 66.903 | 1.00 | 8.87 | C |
| ATOM | 3939 | OG | SER | C | 154 | 38.718 | 54.529 | 68.016 | 1.00 | 9.50 | C |
| ATOM | 3940 | C | SER | C | 154 | 37.543 | 56.581 | 66.438 | 1.00 | 7.68 | C |
| ATOM | 3941 | O | SER | C | 154 | 38.002 | 57.550 | 67.046 | 1.00 | 9.02 | C |
| ATOM | 3942 | N | ALA | C | 155 | 36.242 | 56.404 | 66.254 | 1.00 | 8.74 | C |
| ATOM | 3943 | CA | ALA | C | 155 | 35.283 | 57.337 | 66.828 | 1.00 | 8.21 | C |
| ATOM | 3944 | CB | ALA | C | 155 | 33.885 | 57.051 | 66.296 | 1.00 | 8.47 | C |
| ATOM | 3945 | C | ALA | C | 155 | 35.322 | 57.157 | 68.343 | 1.00 | 10.66 | C |
| ATOM | 3946 | O | ALA | C | 155 | 35.002 | 58.076 | 69.101 | 1.00 | 11.03 | C |
| ATOM | 3947 | N | LYS | C | 156 | 35.728 | 55.969 | 68.786 | 1.00 | 10.50 | C |
| ATOM | 3948 | CA | LYS | C | 156 | 35.807 | 55.695 | 70.216 | 1.00 | 13.69 | C |
| ATOM | 3949 | CB | LYS | C | 156 | 35.999 | 54.196 | 70.472 | 1.00 | 15.86 | C |
| ATOM | 3950 | CG | LYS | C | 156 | 36.174 | 53.847 | 71.951 | 1.00 | 20.30 | C |
| ATOM | 3951 | CD | LYS | C | 156 | 36.586 | 52.391 | 72.142 | 1.00 | 22.56 | C |
| ATOM | 3952 | CE | LYS | C | 156 | 36.857 | 52.071 | 73.606 | 1.00 | 24.80 | C |
| ATOM | 3953 | NZ | LYS | C | 156 | 35.629 | 52.161 | 74.437 | 1.00 | 25.21 | C |
| ATOM | 3954 | C | LYS | C | 156 | 36.942 | 56.471 | 70.878 | 1.00 | 13.97 | C |
| ATOM | 3955 | O | LYS | C | 156 | 36.734 | 57.126 | 71.903 | 1.00 | 15.24 | C |
| ATOM | 3956 | N | ASP | C | 157 | 38.137 | 56.404 | 70.291 | 1.00 | 14.65 | C |
| ATOM | 3957 | CA | ASP | C | 157 | 39.304 | 57.095 | 70.844 | 1.00 | 15.98 | C |
| ATOM | 3958 | CB | ASP | C | 157 | 40.573 | 56.251 | 70.670 | 1.00 | 18.83 | C |
| ATOM | 3959 | CG | ASP | C | 157 | 40.455 | 54.872 | 71.283 | 1.00 | 22.17 | C |
| ATOM | 3960 | OD1 | ASP | C | 157 | 39.681 | 54.708 | 72.251 | 1.00 | 25.86 | C |
| ATOM | 3961 | OD2 | ASP | C | 157 | 41.154 | 53.954 | 70.803 | 1.00 | 24.39 | C |
| ATOM | 3962 | C | ASP | C | 157 | 39.561 | 58.466 | 70.221 | 1.00 | 15.42 | C |
| ATOM | 3963 | O | ASP | C | 157 | 40.468 | 59.176 | 70.649 | 1.00 | 16.12 | C |
| ATOM | 3964 | N | ALA | C | 158 | 38.775 | 58.818 | 69.208 | 1.00 | 14.97 | C |
| ATOM | 3965 | CA | ALA | C | 158 | 38.895 | 60.093 | 68.495 | 1.00 | 14.59 | C |
| ATOM | 3966 | CB | ALA | C | 158 | 38.938 | 61.258 | 69.490 | 1.00 | 16.20 | C |
| ATOM | 3967 | C | ALA | C | 158 | 40.110 | 60.155 | 67.566 | 1.00 | 14.58 | C |
| ATOM | 3968 | O | ALA | C | 158 | 40.377 | 61.188 | 66.956 | 1.00 | 15.40 | C |
| ATOM | 3969 | N | THR | C | 159 | 40.832 | 59.046 | 67.448 | 1.00 | 13.26 | C |
| ATOM | 3970 | CA | THR | C | 159 | 42.014 | 58.989 | 66.598 | 1.00 | 12.03 | C |
| ATOM | 3971 | CB | THR | C | 159 | 42.586 | 57.555 | 66.544 | 1.00 | 12.45 | C |
| ATOM | 3972 | OG1 | THR | C | 159 | 42.763 | 57.057 | 67.876 | 1.00 | 13.56 | C |
| ATOM | 3973 | CG2 | THR | C | 159 | 43.926 | 57.543 | 65.832 | 1.00 | 13.12 | C |
| ATOM | 3974 | C | THR | C | 159 | 41.738 | 59.447 | 65.158 | 1.00 | 12.04 | C |
| ATOM | 3975 | O | THR | C | 159 | 40.880 | 58.886 | 64.480 | 1.00 | 10.84 | C |
| ATOM | 3976 | N | ASN | C | 160 | 42.465 | 60.470 | 64.712 | 1.00 | 10.63 | C |
| ATOM | 3977 | CA | ASN | C | 160 | 42.346 | 61.003 | 63.351 | 1.00 | 11.35 | C |
| ATOM | 3978 | CB | ASN | C | 160 | 42.840 | 59.967 | 62.329 | 1.00 | 13.04 | C |
| ATOM | 3979 | CG | ASN | C | 160 | 44.351 | 59.811 | 62.330 | 1.00 | 15.32 | C |
| ATOM | 3980 | OD1 | ASN | C | 160 | 45.087 | 60.798 | 62.401 | 1.00 | 16.07 | C |
| ATOM | 3981 | ND2 | ASN | C | 160 | 44.821 | 58.571 | 62.231 | 1.00 | 14.55 | C |
| ATOM | 3982 | C | ASN | C | 160 | 40.965 | 61.486 | 62.905 | 1.00 | 10.69 | C |
| ATOM | 3983 | O | ASN | C | 160 | 40.760 | 61.760 | 61.721 | 1.00 | 11.22 | C |
| ATOM | 3984 | N | VAL | C | 161 | 40.021 | 61.603 | 63.831 | 1.00 | 10.61 | C |
| ATOM | 3985 | CA | VAL | C | 161 | 38.682 | 62.043 | 63.464 | 1.00 | 11.24 | C |
| ATOM | 3986 | CB | VAL | C | 161 | 37.709 | 61.875 | 64.642 | 1.00 | 10.84 | C |
| ATOM | 3987 | CG1 | VAL | C | 161 | 36.333 | 62.415 | 64.273 | 1.00 | 11.65 | C |
| ATOM | 3988 | CG2 | VAL | C | 161 | 37.614 | 60.401 | 65.008 | 1.00 | 10.60 | C |
| ATOM | 3989 | C | VAL | C | 161 | 38.645 | 63.483 | 62.962 | 1.00 | 11.66 | C |
| ATOM | 3990 | O | VAL | C | 161 | 38.165 | 63.747 | 61.856 | 1.00 | 11.18 | C |
| ATOM | 3991 | N | ALA | C | 162 | 39.152 | 64.415 | 63.764 | 1.00 | 11.55 | C |
| ATOM | 3992 | CA | ALA | C | 162 | 39.168 | 65.816 | 63.356 | 1.00 | 12.36 | C |
| ATOM | 3993 | CB | ALA | C | 162 | 39.734 | 66.691 | 64.480 | 1.00 | 11.40 | C |
| ATOM | 3994 | C | ALA | C | 162 | 40.008 | 65.971 | 62.090 | 1.00 | 12.34 | C |
| ATOM | 3995 | O | ALA | C | 162 | 39.645 | 66.714 | 61.178 | 1.00 | 12.02 | C |
| ATOM | 3996 | N | ALA | C | 163 | 41.129 | 65.259 | 62.033 | 1.00 | 12.52 | C |
| ATOM | 3997 | CA | ALA | C | 163 | 42.010 | 65.328 | 60.871 | 1.00 | 13.51 | C |
| ATOM | 3998 | CB | ALA | C | 163 | 43.203 | 64.401 | 61.063 | 1.00 | 14.58 | C |
| ATOM | 3999 | C | ALA | C | 163 | 41.270 | 64.960 | 59.588 | 1.00 | 13.79 | C |
| ATOM | 4000 | O | ALA | C | 163 | 41.394 | 65.645 | 58.570 | 1.00 | 12.63 | C |
| ATOM | 4001 | N | ALA | C | 164 | 40.495 | 63.879 | 59.646 | 1.00 | 12.39 | C |
| ATOM | 4002 | CA | ALA | C | 164 | 39.741 | 63.404 | 58.487 | 1.00 | 13.53 | C |
| ATOM | 4003 | CB | ALA | C | 164 | 39.060 | 62.084 | 58.816 | 1.00 | 12.16 | C |
| ATOM | 4004 | C | ALA | C | 164 | 38.706 | 64.411 | 57.999 | 1.00 | 13.82 | C |
| ATOM | 4005 | O | ALA | C | 164 | 38.607 | 64.673 | 56.799 | 1.00 | 13.24 | C |
| ATOM | 4006 | N | PHE | C | 165 | 37.931 | 64.959 | 58.930 | 1.00 | 13.35 | C |
| ATOM | 4007 | CA | PHE | C | 165 | 36.907 | 65.939 | 58.594 | 1.00 | 15.63 | C |
| ATOM | 4008 | CB | PHE | C | 165 | 36.029 | 66.234 | 59.814 | 1.00 | 16.76 | C |
| ATOM | 4009 | CG | PHE | C | 165 | 34.973 | 65.192 | 60.069 | 1.00 | 18.55 | C |
| ATOM | 4010 | CD1 | PHE | C | 165 | 33.796 | 65.182 | 59.329 | 1.00 | 19.01 | C |
| ATOM | 4011 | CD2 | PHE | C | 165 | 35.161 | 64.214 | 61.039 | 1.00 | 18.66 | C |
| ATOM | 4012 | CE1 | PHE | C | 165 | 32.820 | 64.214 | 59.551 | 1.00 | 20.32 | C |
| ATOM | 4013 | CE2 | PHE | C | 165 | 34.194 | 63.240 | 61.269 | 1.00 | 21.11 | C |
| ATOM | 4014 | CZ | PHE | C | 165 | 33.019 | 63.239 | 60.524 | 1.00 | 21.48 | C |
| ATOM | 4015 | C | PHE | C | 165 | 37.528 | 67.233 | 58.077 | 1.00 | 16.34 | C |
| ATOM | 4016 | O | PHE | C | 165 | 37.039 | 67.814 | 57.110 | 1.00 | 17.31 | C |
| ATOM | 4017 | N | GLU | C | 166 | 38.600 | 67.683 | 58.722 | 1.00 | 17.17 | C |
| ATOM | 4018 | CA | GLU | C | 166 | 39.273 | 68.908 | 58.300 | 1.00 | 19.47 | C |
| ATOM | 4019 | CB | GLU | C | 166 | 40.397 | 69.266 | 59.274 | 1.00 | 21.29 | C |
| ATOM | 4020 | CG | GLU | C | 166 | 39.925 | 69.638 | 60.672 | 1.00 | 26.75 | C |
| ATOM | 4021 | CD | GLU | C | 166 | 41.078 | 69.863 | 61.635 | 1.00 | 29.28 | C |
| ATOM | 4022 | OE1 | GLU | C | 166 | 40.819 | 70.194 | 62.811 | 1.00 | 31.69 | C |
| ATOM | 4023 | OE2 | GLU | C | 166 | 42.245 | 69.704 | 61.220 | 1.00 | 32.72 | C |
| ATOM | 4024 | C | GLU | C | 166 | 39.853 | 68.720 | 56.900 | 1.00 | 18.87 | C |
| ATOM | 4025 | O | GLU | C | 166 | 39.801 | 69.625 | 56.065 | 1.00 | 16.34 | C |
| ATOM | 4026 | N | GLU | C | 167 | 40.415 | 67.542 | 56.651 | 1.00 | 17.33 | C |
| ATOM | 4027 | CA | GLU | C | 167 | 40.999 | 67.249 | 55.352 | 1.00 | 18.66 | C |
| ATOM | 4028 | CB | GLU | C | 167 | 41.583 | 65.834 | 55.337 | 1.00 | 18.32 | C |
| ATOM | 4029 | CG | GLU | C | 167 | 42.174 | 65.391 | 54.000 | 1.00 | 19.04 | C |
| ATOM | 4030 | CD | GLU | C | 167 | 43.239 | 66.334 | 53.466 | 1.00 | 19.96 | C |
| ATOM | 4031 | OE1 | GLU | C | 167 | 43.880 | 67.040 | 54.274 | 1.00 | 21.01 | C |
| ATOM | 4032 | OE2 | GLU | C | 167 | 43.443 | 66.358 | 52.232 | 1.00 | 19.77 | C |
| ATOM | 4033 | C | GLU | C | 167 | 39.928 | 67.388 | 54.285 | 1.00 | 19.53 | C |
| ATOM | 4034 | O | GLU | C | 167 | 40.202 | 67.839 | 53.177 | 1.00 | 18.58 | C |
| ATOM | 4035 | N | ALA | C | 168 | 38.703 | 67.005 | 54.629 | 1.00 | 21.20 | C |
| ATOM | 4036 | CA | ALA | C | 168 | 37.594 | 67.099 | 53.688 | 1.00 | 22.96 | C |
| ATOM | 4037 | CB | ALA | C | 168 | 36.297 | 66.676 | 54.359 | 1.00 | 23.82 | C |
| ATOM | 4038 | C | ALA | C | 168 | 37.488 | 68.534 | 53.185 | 1.00 | 24.15 | C |
| ATOM | 4039 | O | ALA | C | 168 | 37.505 | 68.774 | 51.978 | 1.00 | 23.72 | C |
| ATOM | 4040 | N | VAL | C | 169 | 37.389 | 69.484 | 54.114 | 1.00 | 25.80 | C |
| ATOM | 4041 | CA | VAL | C | 169 | 37.292 | 70.900 | 53.760 | 1.00 | 26.44 | C |
| ATOM | 4042 | CB | VAL | C | 169 | 37.107 | 71.792 | 55.012 | 1.00 | 26.83 | C |
| ATOM | 4043 | CG1 | VAL | C | 169 | 37.137 | 73.265 | 54.616 | 1.00 | 26.48 | C |
| ATOM | 4044 | CG2 | VAL | C | 169 | 35.787 | 71.465 | 55.689 | 1.00 | 28.08 | C |
| ATOM | 4045 | C | VAL | C | 169 | 38.544 | 71.359 | 53.022 | 1.00 | 27.04 | C |
| ATOM | 4046 | O | VAL | C | 169 | 38.477 | 72.203 | 52.130 | 1.00 | 26.03 | C |
| ATOM | 4047 | N | ARG | C | 170 | 39.688 | 70.803 | 53.406 | 1.00 | 28.26 | C |
| ATOM | 4048 | CA | ARG | C | 170 | 40.950 | 71.146 | 52.767 | 1.00 | 28.96 | C |
| ATOM | 4049 | CB | ARG | C | 170 | 42.102 | 70.396 | 53.439 | 1.00 | 30.55 | C |
| ATOM | 4050 | CG | ARG | C | 170 | 43.456 | 70.622 | 52.786 | 1.00 | 33.22 | C |
| ATOM | 4051 | CD | ARG | C | 170 | 43.911 | 72.063 | 52.934 | 1.00 | 36.59 | C |
| ATOM | 4052 | NE | ARG | C | 170 | 44.225 | 72.405 | 54.320 | 1.00 | 37.71 | C |
| ATOM | 4053 | CZ | ARG | C | 170 | 44.610 | 73.615 | 54.717 | 1.00 | 37.76 | C |
| ATOM | 4054 | NH1 | ARG | C | 170 | 44.724 | 74.597 | 53.832 | 1.00 | 37.12 | C |
| ATOM | 4055 | NH2 | ARG | C | 170 | 44.884 | 73.844 | 55.997 | 1.00 | 36.75 | C |
| ATOM | 4056 | C | ARG | C | 170 | 40.881 | 70.771 | 51.288 | 1.00 | 28.85 | C |
| ATOM | 4057 | O | ARG | C | 170 | 41.238 | 71.567 | 50.416 | 1.00 | 28.51 | C |
| ATOM | 4058 | N | ARG | C | 171 | 40.421 | 69.553 | 51.016 | 1.00 | 27.96 | C |
| ATOM | 4059 | CA | ARG | C | 171 | 40.295 | 69.064 | 49.646 | 1.00 | 28.63 | C |
| ATOM | 4060 | CB | ARG | C | 171 | 39.778 | 67.621 | 49.628 | 1.00 | 28.97 | C |
| ATOM | 4061 | CG | ARG | C | 171 | 40.776 | 66.562 | 50.079 | 1.00 | 28.80 | C |
| ATOM | 4062 | CD | ARG | C | 171 | 41.999 | 66.506 | 49.172 | 1.00 | 30.09 | C |
| ATOM | 4063 | NE | ARG | C | 171 | 41.650 | 66.418 | 47.755 | 1.00 | 31.80 | C |
| ATOM | 4064 | CZ | ARG | C | 171 | 41.013 | 65.395 | 47.187 | 1.00 | 33.34 | C |
| ATOM | 4065 | NH1 | ARG | C | 171 | 40.639 | 64.341 | 47.906 | 1.00 | 28.26 | C |
| ATOM | 4066 | NH2 | ARG | C | 171 | 40.744 | 65.431 | 45.886 | 1.00 | 34.50 | C |
| ATOM | 4067 | C | ARG | C | 171 | 39.345 | 69.945 | 48.846 | 1.00 | 29.27 | C |
| ATOM | 4068 | O | ARG | C | 171 | 39.562 | 70.179 | 47.657 | 1.00 | 29.19 | C |
| ATOM | 4069 | N | VAL | C | 172 | 38.287 | 70.426 | 49.494 | 1.00 | 29.98 | C |
| ATOM | 4070 | CA | VAL | C | 172 | 37.325 | 71.287 | 48.814 | 1.00 | 31.19 | C |
| ATOM | 4071 | CB | VAL | C | 172 | 36.122 | 71.639 | 49.719 | 1.00 | 31.40 | C |
| ATOM | 4072 | CG1 | VAL | C | 172 | 35.126 | 72.482 | 48.936 | 1.00 | 32.02 | C |
| ATOM | 4073 | CG2 | VAL | C | 172 | 35.457 | 70.372 | 50.230 | 1.00 | 31.49 | C |
| ATOM | 4074 | C | VAL | C | 172 | 38.007 | 72.586 | 48.395 | 1.00 | 31.44 | C |
| ATOM | 4075 | O | VAL | C | 172 | 37.895 | 73.012 | 47.245 | 1.00 | 32.14 | C |
| ATOM | 4076 | N | LEU | C | 173 | 38.714 | 73.206 | 49.335 | 1.00 | 31.46 | C |
| ATOM | 4077 | CA | LEU | C | 173 | 39.421 | 74.456 | 49.074 | 1.00 | 32.50 | C |
| ATOM | 4078 | CB | LEU | C | 173 | 40.123 | 74.944 | 50.344 | 1.00 | 32.76 | C |
| ATOM | 4079 | CG | LEU | C | 173 | 39.233 | 75.403 | 51.501 | 1.00 | 33.84 | C |
| ATOM | 4080 | CD1 | LEU | C | 173 | 40.100 | 75.810 | 52.680 | 1.00 | 34.92 | C |
| ATOM | 4081 | CD2 | LEU | C | 173 | 38.371 | 76.571 | 51.054 | 1.00 | 34.84 | C |
| ATOM | 4082 | C | LEU | C | 173 | 40.448 | 74.318 | 47.957 | 1.00 | 33.07 | C |
| ATOM | 4083 | O | LEU | C | 173 | 40.757 | 75.289 | 47.266 | 1.00 | 32.93 | C |
| ATOM | 4084 | N | ALA | C | 174 | 40.980 | 73.112 | 47.787 | 1.00 | 33.35 | C |
| ATOM | 4085 | CA | ALA | C | 174 | 41.976 | 72.856 | 46.752 | 1.00 | 33.98 | C |
| ATOM | 4086 | CB | ALA | C | 174 | 42.895 | 71.719 | 47.183 | 1.00 | 33.16 | C |
| ATOM | 4087 | C | ALA | C | 174 | 41.320 | 72.519 | 45.417 | 1.00 | 34.39 | C |
| ATOM | 4088 | O | ALA | C | 174 | 40.074 | 72.434 | 45.373 | 1.00 | 35.41 | C |
| ATOM | 4089 | OXT | ALA | C | 174 | 42.064 | 72.339 | 44.430 | 1.00 | 35.60 | C |
| TER | | | | | | | | | | | |
| ATOM | 4090 | CB | SER | D | 6 | 3.433 | 58.150 | 46.248 | 1.00 | 17.85 | D |
| ATOM | 4091 | OG | SER | D | 6 | 4.653 | 57.452 | 46.077 | 1.00 | 21.36 | D |
| ATOM | 4092 | C | SER | D | 6 | 4.563 | 59.972 | 47.518 | 1.00 | 16.69 | D |
| ATOM | 4093 | O | SER | D | 6 | 4.377 | 59.415 | 48.604 | 1.00 | 15.36 | D |
| ATOM | 4094 | N | SER | D | 6 | 4.279 | 60.145 | 45.045 | 1.00 | 18.67 | D |
| ATOM | 4095 | CA | SER | D | 6 | 3.674 | 59.659 | 46.319 | 1.00 | 17.61 | D |
| ATOM | 4096 | N | LEU | D | 7 | 5.533 | 60.857 | 47.315 | 1.00 | 15.61 | D |
| ATOM | 4097 | CA | LEU | D | 7 | 6.440 | 61.251 | 48.387 | 1.00 | 14.95 | D |
| ATOM | 4098 | CB | LEU | D | 7 | 7.878 | 61.352 | 47.866 | 1.00 | 15.23 | D |
| ATOM | 4099 | CG | LEU | D | 7 | 8.962 | 61.638 | 48.913 | 1.00 | 14.07 | D |
| ATOM | 4100 | CD1 | LEU | D | 7 | 8.984 | 60.523 | 49.955 | 1.00 | 13.87 | D |
| ATOM | 4101 | CD2 | LEU | D | 7 | 10.319 | 61.747 | 48.227 | 1.00 | 16.29 | D |
| ATOM | 4102 | C | LEU | D | 7 | 5.995 | 62.605 | 48.927 | 1.00 | 14.96 | D |
| ATOM | 4103 | O | LEU | D | 7 | 6.162 | 63.629 | 48.265 | 1.00 | 15.92 | D |
| ATOM | 4104 | N | PHE | D | 8 | 5.412 | 62.609 | 50.121 | 1.00 | 12.64 | D |
| ATOM | 4105 | CA | PHE | D | 8 | 4.958 | 63.859 | 50.720 | 1.00 | 11.94 | D |
| ATOM | 4106 | CB | PHE | D | 8 | 3.566 | 63.702 | 51.339 | 1.00 | 12.12 | D |
| ATOM | 4107 | CG | PHE | D | 8 | 2.471 | 63.420 | 50.344 | 1.00 | 12.80 | D |
| ATOM | 4108 | CD1 | PHE | D | 8 | 2.700 | 63.505 | 48.974 | 1.00 | 15.61 | D |
| ATOM | 4109 | CD2 | PHE | D | 8 | 1.198 | 63.079 | 50.789 | 1.00 | 12.62 | D |
| ATOM | 4110 | CE1 | PHE | D | 8 | 1.674 | 63.253 | 48.059 | 1.00 | 15.74 | D |
| ATOM | 4111 | CE2 | PHE | D | 8 | 0.167 | 62.826 | 49.890 | 1.00 | 13.19 | D |
| ATOM | 4112 | CZ | PHE | D | 8 | 0.404 | 62.912 | 48.519 | 1.00 | 15.20 | D |
| ATOM | 4113 | C | PHE | D | 8 | 5.923 | 64.308 | 51.803 | 1.00 | 12.17 | D |
| ATOM | 4114 | O | PHE | D | 8 | 6.103 | 63.625 | 52.811 | 1.00 | 12.02 | D |
| ATOM | 4115 | N | LYS | D | 9 | 6.542 | 65.461 | 51.594 | 1.00 | 10.34 | D |
| ATOM | 4116 | CA | LYS | D | 9 | 7.472 | 66.001 | 52.573 | 1.00 | 10.35 | D |
| ATOM | 4117 | CB | LYS | D | 9 | 8.504 | 66.887 | 51.884 | 1.00 | 10.01 | D |
| ATOM | 4118 | CG | LYS | D | 9 | 9.407 | 67.638 | 52.841 | 1.00 | 10.05 | D |
| ATOM | 4119 | CD | LYS | D | 9 | 10.518 | 68.325 | 52.075 | 1.00 | 12.14 | D |
| ATOM | 4120 | CE | LYS | D | 9 | 11.344 | 69.226 | 52.971 | 1.00 | 13.68 | D |
| ATOM | 4121 | NZ | LYS | D | 9 | 12.409 | 69.905 | 52.177 | 1.00 | 16.60 | D |
| ATOM | 4122 | C | LYS | D | 9 | 6.697 | 66.809 | 53.605 | 1.00 | 10.46 | D |
| ATOM | 4123 | O | LYS | D | 9 | 6.015 | 67.778 | 53.267 | 1.00 | 11.28 | D |
| ATOM | 4124 | N | VAL | D | 10 | 6.794 | 66.392 | 54.863 | 1.00 | 10.35 | D |
| ATOM | 4125 | CA | VAL | D | 10 | 6.100 | 67.066 | 55.952 | 1.00 | 10.77 | D |
| ATOM | 4126 | CB | VAL | D | 10 | 5.127 | 66.103 | 56.664 | 1.00 | 11.61 | D |
| ATOM | 4127 | CG1 | VAL | D | 10 | 4.407 | 66.824 | 57.803 | 1.00 | 11.98 | D |
| ATOM | 4128 | CG2 | VAL | D | 10 | 4.120 | 65.562 | 55.654 | 1.00 | 11.61 | D |
| ATOM | 4129 | C | VAL | D | 10 | 7.142 | 67.571 | 56.933 | 1.00 | 10.54 | D |
| ATOM | 4130 | O | VAL | D | 10 | 7.955 | 66.804 | 57.444 | 1.00 | 10.86 | D |
| ATOM | 4131 | N | ILE | D | 11 | 7.125 | 68.869 | 57.195 | 1.00 | 11.11 | D |
| ATOM | 4132 | CA | ILE | D | 11 | 8.115 | 69.442 | 58.089 | 1.00 | 11.38 | D |
| ATOM | 4133 | CB | ILE | D | 11 | 8.866 | 70.609 | 57.384 | 1.00 | 12.31 | D |
| ATOM | 4134 | CG2 | ILE | D | 11 | 7.896 | 71.726 | 57.058 | 1.00 | 11.97 | D |
| ATOM | 4135 | CG1 | ILE | D | 11 | 10.012 | 71.114 | 58.262 | 1.00 | 12.55 | D |
| ATOM | 4136 | CD1 | ILE | D | 11 | 10.914 | 72.141 | 57.568 | 1.00 | 14.55 | D |
| ATOM | 4137 | C | ILE | D | 11 | 7.506 | 69.921 | 59.399 | 1.00 | 11.82 | D |
| ATOM | 4138 | O | ILE | D | 11 | 6.426 | 70.515 | 59.420 | 1.00 | 11.78 | D |
| ATOM | 4139 | N | LEU | D | 12 | 8.209 | 69.631 | 60.488 | 1.00 | 11.10 | D |
| ATOM | 4140 | CA | LEU | D | 12 | 7.796 | 70.017 | 61.833 | 1.00 | 11.40 | D |
| ATOM | 4141 | CB | LEU | D | 12 | 8.167 | 68.909 | 62.825 | 1.00 | 11.76 | D |
| ATOM | 4142 | CG | LEU | D | 12 | 7.421 | 67.578 | 62.689 | 1.00 | 13.22 | D |
| ATOM | 4143 | CD1 | LEU | D | 12 | 8.229 | 66.450 | 63.314 | 1.00 | 14.56 | D |
| ATOM | 4144 | CD2 | LEU | D | 12 | 6.062 | 67.701 | 63.353 | 1.00 | 15.96 | D |
| ATOM | 4145 | C | LEU | D | 12 | 8.497 | 71.314 | 62.239 | 1.00 | 11.63 | D |
| ATOM | 4146 | O | LEU | D | 12 | 9.724 | 71.364 | 62.303 | 1.00 | 11.06 | D |
| ATOM | 4147 | N | LEU | D | 13 | 7.718 | 72.359 | 62.507 | 1.00 | 10.86 | D |
| ATOM | 4148 | CA | LEU | D | 13 | 8.274 | 73.646 | 62.923 | 1.00 | 12.30 | D |
| ATOM | 4149 | CB | LEU | D | 13 | 7.983 | 74.730 | 61.877 | 1.00 | 13.39 | D |
| ATOM | 4150 | CG | LEU | D | 13 | 8.629 | 74.559 | 60.499 | 1.00 | 15.94 | D |
| ATOM | 4151 | CD1 | LEU | D | 13 | 8.243 | 75.729 | 59.601 | 1.00 | 16.99 | D |
| ATOM | 4152 | CD2 | LEU | D | 13 | 10.138 | 74.482 | 60.646 | 1.00 | 16.37 | D |
| ATOM | 4153 | C | LEU | D | 13 | 7.657 | 74.046 | 64.258 | 1.00 | 11.25 | D |
| ATOM | 4154 | O | LEU | D | 13 | 6.524 | 73.670 | 64.557 | 1.00 | 12.05 | D |
| ATOM | 4155 | N | GLY | D | 14 | 8.409 | 74.800 | 65.056 | 1.00 | 9.77 | D |
| ATOM | 4156 | CA | GLY | D | 14 | 7.923 | 75.236 | 66.354 | 1.00 | 8.40 | D |
| ATOM | 4157 | C | GLY | D | 14 | 9.083 | 75.516 | 67.292 | 1.00 | 9.34 | D |
| ATOM | 4158 | O | GLY | D | 14 | 10.189 | 75.016 | 67.072 | 1.00 | 8.87 | D |
| ATOM | 4159 | N | ASP | D | 15 | 8.842 | 76.310 | 68.333 | 1.00 | 8.78 | D |
| ATOM | 4160 | CA | ASP | D | 15 | 9.888 | 76.663 | 69.292 | 1.00 | 10.42 | D |
| ATOM | 4161 | CB | ASP | D | 15 | 9.304 | 77.471 | 70.459 | 1.00 | 9.30 | D |
| ATOM | 4162 | CG | ASP | D | 15 | 8.931 | 78.894 | 70.068 | 1.00 | 12.08 | D |
| ATOM | 4163 | OD1 | ASP | D | 15 | 9.201 | 79.305 | 68.918 | 1.00 | 12.73 | D |
| ATOM | 4164 | OD2 | ASP | D | 15 | 8.368 | 79.606 | 70.925 | 1.00 | 13.05 | D |
| ATOM | 4165 | C | ASP | D | 15 | 10.614 | 75.450 | 69.863 | 1.00 | 11.03 | D |
| ATOM | 4166 | O | ASP | D | 15 | 10.062 | 74.351 | 69.943 | 1.00 | 11.51 | D |
| ATOM | 4167 | N | GLY | D | 16 | 11.861 | 75.655 | 70.266 | 1.00 | 11.79 | D |
| ATOM | 4168 | CA | GLY | D | 16 | 12.611 | 74.563 | 70.850 | 1.00 | 11.59 | D |
| ATOM | 4169 | C | GLY | D | 16 | 11.932 | 74.131 | 72.139 | 1.00 | 12.14 | D |
| ATOM | 4170 | O | GLY | D | 16 | 11.512 | 74.975 | 72.936 | 1.00 | 11.77 | D |
| ATOM | 4171 | N | GLY | D | 17 | 11.795 | 72.822 | 72.332 | 1.00 | 11.37 | D |
| ATOM | 4172 | CA | GLY | D | 17 | 11.181 | 72.309 | 73.546 | 1.00 | 9.49 | D |
| ATOM | 4173 | C | GLY | D | 17 | 9.707 | 71.934 | 73.506 | 1.00 | 10.15 | D |
| ATOM | 4174 | O | GLY | D | 17 | 9.198 | 71.342 | 74.461 | 1.00 | 9.37 | D |
| ATOM | 4175 | N | VAL | D | 18 | 9.015 | 72.246 | 72.415 | 1.00 | 8.71 | D |
| ATOM | 4176 | CA | VAL | D | 18 | 7.593 | 71.940 | 72.334 | 1.00 | 8.97 | D |
| ATOM | 4177 | CB | VAL | D | 18 | 6.912 | 72.757 | 71.216 | 1.00 | 9.25 | D |
| ATOM | 4178 | CG1 | VAL | D | 18 | 7.088 | 74.253 | 71.500 | 1.00 | 8.64 | D |
| ATOM | 4179 | CG2 | VAL | D | 18 | 7.500 | 72.396 | 69.862 | 1.00 | 11.00 | D |
| ATOM | 4180 | C | VAL | D | 18 | 7.301 | 70.456 | 72.143 | 1.00 | 8.42 | D |
| ATOM | 4181 | O | VAL | D | 18 | 6.194 | 70.000 | 72.422 | 1.00 | 8.69 | D |
| ATOM | 4182 | N | GLY | D | 19 | 8.290 | 69.705 | 71.665 | 1.00 | 8.51 | D |
| ATOM | 4183 | CA | GLY | D | 19 | 8.098 | 68.275 | 71.484 | 1.00 | 8.72 | D |
| ATOM | 4184 | C | GLY | D | 19 | 8.122 | 67.753 | 70.062 | 1.00 | 8.10 | D |
| ATOM | 4185 | O | GLY | D | 19 | 7.587 | 66.675 | 69.804 | 1.00 | 7.64 | D |
| ATOM | 4186 | N | LYS | D | 20 | 8.744 | 68.493 | 69.144 | 1.00 | 6.01 | D |
| ATOM | 4187 | CA | LYS | D | 20 | 8.812 | 68.077 | 67.743 | 1.00 | 7.66 | D |
| ATOM | 4188 | CB | LYS | D | 20 | 9.557 | 69.129 | 66.914 | 1.00 | 7.84 | D |
| ATOM | 4189 | CG | LYS | D | 20 | 8.852 | 70.483 | 66.859 | 1.00 | 10.02 | D |
| ATOM | 4190 | CD | LYS | D | 20 | 9.607 | 71.482 | 65.973 | 1.00 | 9.82 | D |
| ATOM | 4191 | CE | LYS | D | 20 | 11.008 | 71.750 | 66.507 | 1.00 | 11.51 | D |
| ATOM | 4192 | NZ | LYS | D | 20 | 10.970 | 72.254 | 67.908 | 1.00 | 7.85 | D |
| ATOM | 4193 | C | LYS | D | 20 | 9.494 | 66.719 | 67.572 | 1.00 | 8.21 | D |
| ATOM | 4194 | O | LYS | D | 20 | 8.944 | 65.810 | 66.945 | 1.00 | 8.26 | D |
| ATOM | 4195 | N | SER | D | 21 | 10.691 | 66.583 | 68.131 | 1.00 | 7.50 | D |
| ATOM | 4196 | CA | SER | D | 21 | 11.428 | 65.327 | 68.031 | 1.00 | 7.46 | D |
| ATOM | 4197 | CB | SER | D | 21 | 12.814 | 65.474 | 68.654 | 1.00 | 9.16 | D |
| ATOM | 4198 | OG | SER | D | 21 | 13.633 | 66.289 | 67.840 | 1.00 | 9.16 | D |
| ATOM | 4199 | C | SER | D | 21 | 10.686 | 64.181 | 68.700 | 1.00 | 8.04 | D |
| ATOM | 4200 | O | SER | D | 21 | 10.695 | 63.053 | 68.197 | 1.00 | 8.37 | D |
| ATOM | 4201 | N | SER | D | 22 | 10.044 | 64.463 | 69.828 | 1.00 | 7.27 | D |
| ATOM | 4202 | CA | SER | D | 22 | 9.298 | 63.435 | 70.546 | 1.00 | 7.84 | D |
| ATOM | 4203 | CB | SER | D | 22 | 8.830 | 63.960 | 71.909 | 1.00 | 7.02 | D |
| ATOM | 4204 | OG | SER | D | 22 | 9.935 | 64.269 | 72.742 | 1.00 | 7.56 | D |
| ATOM | 4205 | C | SER | D | 22 | 8.094 | 62.976 | 69.727 | 1.00 | 7.65 | D |
| ATOM | 4206 | O | SER | D | 22 | 7.786 | 61.787 | 69.689 | 1.00 | 9.44 | D |
| ATOM | 4207 | N | LEU | D | 23 | 7.413 | 63.915 | 69.073 | 1.00 | 7.71 | D |
| ATOM | 4208 | CA | LEU | D | 23 | 6.259 | 63.566 | 68.257 | 1.00 | 7.13 | D |
| ATOM | 4209 | CB | LEU | D | 23 | 5.568 | 64.830 | 67.724 | 1.00 | 6.67 | D |
| ATOM | 4210 | CG | LEU | D | 23 | 4.707 | 65.573 | 68.751 | 1.00 | 7.88 | D |
| ATOM | 4211 | CD1 | LEU | D | 23 | 4.391 | 66.974 | 68.254 | 1.00 | 8.09 | D |
| ATOM | 4212 | CD2 | LEU | D | 23 | 3.431 | 64.773 | 69.005 | 1.00 | 8.28 | D |
| ATOM | 4213 | C | LEU | D | 23 | 6.694 | 62.682 | 67.096 | 1.00 | 6.63 | D |
| ATOM | 4214 | O | LEU | D | 23 | 6.040 | 61.687 | 66.788 | 1.00 | 5.79 | D |
| ATOM | 4215 | N | MET | D | 24 | 7.798 | 63.043 | 66.451 | 1.00 | 7.18 | D |
| ATOM | 4216 | CA | MET | D | 24 | 8.285 | 62.253 | 65.329 | 1.00 | 9.16 | D |
| ATOM | 4217 | CB | MET | D | 24 | 9.496 | 62.925 | 64.672 | 1.00 | 10.53 | D |
| ATOM | 4218 | CG | MET | D | 24 | 10.097 | 62.120 | 63.519 | 1.00 | 14.30 | D |
| ATOM | 4219 | SD | MET | D | 24 | 11.480 | 62.967 | 62.716 | 1.00 | 19.01 | D |
| ATOM | 4220 | CE | MET | D | 24 | 12.801 | 62.589 | 63.857 | 1.00 | 21.90 | D |
| ATOM | 4221 | C | MET | D | 24 | 8.667 | 60.857 | 65.795 | 1.00 | 8.06 | D |
| ATOM | 4222 | O | MET | D | 24 | 8.300 | 59.859 | 65.171 | 1.00 | 8.63 | D |
| ATOM | 4223 | N | ASN | D | 25 | 9.398 | 60.782 | 66.898 | 1.00 | 9.26 | D |
| ATOM | 4224 | CA | ASN | D | 25 | 9.825 | 59.484 | 67.401 | 1.00 | 9.22 | D |
| ATOM | 4225 | CB | ASN | D | 25 | 10.844 | 59.647 | 68.524 | 1.00 | 12.43 | D |
| ATOM | 4226 | CG | ASN | D | 25 | 11.511 | 58.337 | 68.880 | 1.00 | 16.32 | D |
| ATOM | 4227 | OD1 | ASN | D | 25 | 11.865 | 57.554 | 67.995 | 1.00 | 18.75 | D |
| ATOM | 4228 | ND2 | ASN | D | 25 | 11.694 | 58.092 | 70.172 | 1.00 | 19.44 | D |
| ATOM | 4229 | C | ASN | D | 25 | 8.669 | 58.620 | 67.884 | 1.00 | 8.58 | D |
| ATOM | 4230 | O | ASN | D | 25 | 8.699 | 57.399 | 67.728 | 1.00 | 8.68 | D |
| ATOM | 4231 | N | ARG | D | 26 | 7.649 | 59.241 | 68.467 | 1.00 | 7.04 | D |
| ATOM | 4232 | CA | ARG | D | 26 | 6.504 | 58.479 | 68.951 | 1.00 | 7.00 | D |
| ATOM | 4233 | CB | ARG | D | 26 | 5.556 | 59.372 | 69.763 | 1.00 | 7.65 | D |
| ATOM | 4234 | CG | ARG | D | 26 | 4.441 | 58.596 | 70.460 | 1.00 | 13.85 | D |
| ATOM | 4235 | CD | ARG | D | 26 | 5.031 | 57.683 | 71.531 | 1.00 | 14.93 | D |
| ATOM | 4236 | NE | ARG | D | 26 | 4.138 | 56.594 | 71.908 | 1.00 | 17.58 | D |
| ATOM | 4237 | CZ | ARG | D | 26 | 3.638 | 56.424 | 73.126 | 1.00 | 17.51 | D |
| ATOM | 4238 | NH1 | ARG | D | 26 | 3.938 | 57.278 | 74.096 | 1.00 | 17.94 | D |
| ATOM | 4239 | NH2 | ARG | D | 26 | 2.845 | 55.391 | 73.376 | 1.00 | 21.03 | D |
| ATOM | 4240 | C | ARG | D | 26 | 5.747 | 57.867 | 67.772 | 1.00 | 5.73 | D |
| ATOM | 4241 | O | ARG | D | 26 | 5.266 | 56.738 | 67.848 | 1.00 | 6.32 | D |
| ATOM | 4242 | N | TYR | D | 27 | 5.649 | 58.614 | 66.680 | 1.00 | 6.03 | D |
| ATOM | 4243 | CA | TYR | D | 27 | 4.941 | 58.139 | 65.492 | 1.00 | 5.15 | D |
| ATOM | 4244 | CB | TYR | D | 27 | 4.742 | 59.303 | 64.516 | 1.00 | 6.12 | D |
| ATOM | 4245 | CG | TYR | D | 27 | 3.851 | 58.998 | 63.331 | 1.00 | 6.67 | D |
| ATOM | 4246 | CD1 | TYR | D | 27 | 2.608 | 58.384 | 63.502 | 1.00 | 8.63 | D |
| ATOM | 4247 | CE1 | TYR | D | 27 | 1.752 | 58.174 | 62.420 | 1.00 | 9.90 | D |
| ATOM | 4248 | CD2 | TYR | D | 27 | 4.220 | 59.387 | 62.042 | 1.00 | 8.57 | D |
| ATOM | 4249 | CE2 | TYR | D | 27 | 3.371 | 59.185 | 60.957 | 1.00 | 7.71 | D |
| ATOM | 4250 | CZ | TYR | D | 27 | 2.142 | 58.584 | 61.153 | 1.00 | 8.10 | D |
| ATOM | 4251 | OH | TYR | D | 27 | 1.289 | 58.423 | 60.085 | 1.00 | 10.60 | D |
| ATOM | 4252 | C | TYR | D | 27 | 5.704 | 57.015 | 64.791 | 1.00 | 6.82 | D |
| ATOM | 4253 | O | TYR | D | 27 | 5.115 | 56.037 | 64.330 | 1.00 | 5.73 | D |
| ATOM | 4254 | N | VAL | D | 28 | 7.023 | 57.154 | 64.732 | 1.00 | 6.80 | D |
| ATOM | 4255 | CA | VAL | D | 28 | 7.870 | 56.180 | 64.049 | 1.00 | 8.12 | D |
| ATOM | 4256 | CB | VAL | D | 28 | 9.200 | 56.850 | 63.619 | 1.00 | 8.42 | D |
| ATOM | 4257 | CG1 | VAL | D | 28 | 10.109 | 55.838 | 62.931 | 1.00 | 8.85 | D |
| ATOM | 4258 | CG2 | VAL | D | 28 | 8.909 | 58.018 | 62.692 | 1.00 | 9.71 | D |
| ATOM | 4259 | C | VAL | D | 28 | 8.191 | 54.890 | 64.810 | 1.00 | 8.58 | D |
| ATOM | 4260 | O | VAL | D | 28 | 8.186 | 53.805 | 64.221 | 1.00 | 11.34 | D |
| ATOM | 4261 | N | THR | D | 29 | 8.459 | 54.995 | 66.106 | 1.00 | 8.97 | D |
| ATOM | 4262 | CA | THR | D | 29 | 8.815 | 53.820 | 66.904 | 1.00 | 10.27 | D |
| ATOM | 4263 | CB | THR | D | 29 | 10.241 | 53.952 | 67.455 | 1.00 | 12.25 | D |
| ATOM | 4264 | OG1 | THR | D | 29 | 10.272 | 55.004 | 68.429 | 1.00 | 11.99 | D |
| ATOM | 4265 | CG2 | THR | D | 29 | 11.214 | 54.292 | 66.342 | 1.00 | 14.94 | D |
| ATOM | 4266 | C | THR | D | 29 | 7.911 | 53.571 | 68.102 | 1.00 | 9.68 | D |
| ATOM | 4267 | O | THR | D | 29 | 8.056 | 52.562 | 68.791 | 1.00 | 8.40 | D |
| ATOM | 4268 | N | ASN | D | 30 | 6.986 | 54.491 | 68.345 | 1.00 | 8.80 | D |
| ATOM | 4269 | CA | ASN | D | 30 | 6.072 | 54.420 | 69.481 | 1.00 | 10.32 | D |
| ATOM | 4270 | CB | ASN | D | 30 | 5.188 | 53.169 | 69.417 | 1.00 | 10.91 | D |
| ATOM | 4271 | CG | ASN | D | 30 | 4.016 | 53.237 | 70.389 | 1.00 | 11.33 | D |
| ATOM | 4272 | OD1 | ASN | D | 30 | 3.368 | 54.278 | 70.520 | 1.00 | 10.48 | D |
| ATOM | 4273 | ND2 | ASN | D | 30 | 3.730 | 52.127 | 71.064 | 1.00 | 11.56 | D |
| ATOM | 4274 | C | ASN | D | 30 | 6.830 | 54.455 | 70.804 | 1.00 | 9.93 | D |
| ATOM | 4275 | O | ASN | D | 30 | 6.398 | 53.871 | 71.798 | 1.00 | 11.40 | D |
| ATOM | 4276 | N | LYS | D | 31 | 7.971 | 55.139 | 70.811 | 1.00 | 10.58 | D |
| ATOM | 4277 | CA | LYS | D | 31 | 8.767 | 55.272 | 72.028 | 1.00 | 11.45 | D |
| ATOM | 4278 | CB | LYS | D | 31 | 10.210 | 54.826 | 71.789 | 1.00 | 11.37 | D |
| ATOM | 4279 | CG | LYS | D | 31 | 10.360 | 53.342 | 71.496 | 1.00 | 10.79 | D |
| ATOM | 4280 | CD | LYS | D | 31 | 11.803 | 52.988 | 71.211 | 1.00 | 10.80 | D |
| ATOM | 4281 | CE | LYS | D | 31 | 11.964 | 51.495 | 70.969 | 1.00 | 11.03 | D |
| ATOM | 4282 | NZ | LYS | D | 31 | 13.383 | 51.139 | 70.693 | 1.00 | 13.86 | D |
| ATOM | 4283 | C | LYS | D | 31 | 8.748 | 56.729 | 72.473 | 1.00 | 12.51 | D |
| ATOM | 4284 | O | LYS | D | 31 | 8.482 | 57.625 | 71.671 | 1.00 | 9.22 | D |
| ATOM | 4285 | N | PHE | D | 32 | 9.031 | 56.953 | 73.753 | 1.00 | 12.46 | D |
| ATOM | 4286 | CA | PHE | D | 32 | 9.050 | 58.297 | 74.323 | 1.00 | 12.65 | D |
| ATOM | 4287 | CB | PHE | D | 32 | 7.693 | 58.621 | 74.950 | 1.00 | 12.12 | D |
| ATOM | 4288 | CG | PHE | D | 32 | 7.657 | 59.945 | 75.658 | 1.00 | 11.97 | D |
| ATOM | 4289 | CD1 | PHE | D | 32 | 7.615 | 61.132 | 74.934 | 1.00 | 10.55 | D |
| ATOM | 4290 | CD2 | PHE | D | 32 | 7.680 | 60.004 | 77.050 | 1.00 | 11.62 | D |
| ATOM | 4291 | CE1 | PHE | D | 32 | 7.595 | 62.363 | 75.586 | 1.00 | 12.87 | D |
| ATOM | 4292 | CE2 | PHE | D | 32 | 7.662 | 61.233 | 77.715 | 1.00 | 12.40 | D |
| ATOM | 4293 | CZ | PHE | D | 32 | 7.619 | 62.414 | 76.978 | 1.00 | 13.04 | D |
| ATOM | 4294 | C | PHE | D | 32 | 10.134 | 58.406 | 75.392 | 1.00 | 14.54 | D |
| ATOM | 4295 | O | PHE | D | 32 | 10.296 | 57.498 | 76.213 | 1.00 | 13.80 | D |
| ATOM | 4296 | N | ASP | D | 33 | 10.867 | 59.517 | 75.369 | 1.00 | 14.35 | D |
| ATOM | 4297 | CA | ASP | D | 33 | 11.935 | 59.781 | 76.331 | 1.00 | 16.09 | D |
| ATOM | 4298 | CB | ASP | D | 33 | 13.269 | 60.012 | 75.617 | 1.00 | 17.70 | D |
| ATOM | 4299 | CG | ASP | D | 33 | 13.714 | 58.819 | 74.797 | 1.00 | 20.60 | D |
| ATOM | 4300 | OD1 | ASP | D | 33 | 13.815 | 57.708 | 75.362 | 1.00 | 21.42 | D |
| ATOM | 4301 | OD2 | ASP | D | 33 | 13.974 | 58.999 | 73.588 | 1.00 | 23.29 | D |
| ATOM | 4302 | C | ASP | D | 33 | 11.596 | 61.035 | 77.130 | 1.00 | 16.16 | D |
| ATOM | 4303 | O | ASP | D | 33 | 11.341 | 62.092 | 76.548 | 1.00 | 16.12 | D |
| ATOM | 4304 | N | THR | D | 34 | 11.599 | 60.914 | 78.454 | 1.00 | 15.96 | D |
| ATOM | 4305 | CA | THR | D | 34 | 11.304 | 62.045 | 79.335 | 1.00 | 17.67 | D |
| ATOM | 4306 | CB | THR | D | 34 | 11.075 | 61.587 | 80.788 | 1.00 | 17.92 | D |
| ATOM | 4307 | OG1 | THR | D | 34 | 12.222 | 60.859 | 81.238 | 1.00 | 19.73 | D |
| ATOM | 4308 | CG2 | THR | D | 34 | 9.842 | 60.698 | 80.890 | 1.00 | 19.56 | D |
| ATOM | 4309 | C | THR | D | 34 | 12.451 | 63.058 | 79.345 | 1.00 | 18.03 | D |
| ATOM | 4310 | O | THR | D | 34 | 12.233 | 64.255 | 79.547 | 1.00 | 18.10 | D |
| ATOM | 4311 | N | GLN | D | 35 | 13.674 | 62.579 | 79.146 | 1.00 | 17.85 | D |
| ATOM | 4312 | CA | GLN | D | 35 | 14.827 | 63.472 | 79.135 | 1.00 | 19.13 | D |
| ATOM | 4313 | CB | GLN | D | 35 | 15.833 | 63.072 | 80.220 | 1.00 | 19.89 | D |
| ATOM | 4314 | CG | GLN | D | 35 | 15.286 | 63.072 | 81.650 | 1.00 | 21.39 | D |
| ATOM | 4315 | CD | GLN | D | 35 | 14.940 | 64.459 | 82.188 | 1.00 | 23.22 | D |
| ATOM | 4316 | OE1 | GLN | D | 35 | 14.667 | 64.615 | 83.378 | 1.00 | 24.97 | D |
| ATOM | 4317 | NE2 | GLN | D | 35 | 14.946 | 65.465 | 81.319 | 1.00 | 21.78 | D |
| ATOM | 4318 | C | GLN | D | 35 | 15.513 | 63.462 | 77.775 | 1.00 | 19.36 | D |
| ATOM | 4319 | O | GLN | D | 35 | 15.948 | 62.419 | 77.292 | 1.00 | 20.09 | D |
| ATOM | 4320 | N | LEU | D | 36 | 15.601 | 64.636 | 77.163 | 1.00 | 18.11 | D |
| ATOM | 4321 | CA | LEU | D | 36 | 16.234 | 64.786 | 75.862 | 1.00 | 18.42 | D |
| ATOM | 4322 | CB | LEU | D | 36 | 15.174 | 64.854 | 74.757 | 1.00 | 18.10 | D |
| ATOM | 4323 | CG | LEU | D | 36 | 14.400 | 63.578 | 74.416 | 1.00 | 18.22 | D |
| ATOM | 4324 | CD1 | LEU | D | 36 | 13.245 | 63.901 | 73.473 | 1.00 | 16.23 | D |
| ATOM | 4325 | CD2 | LEU | D | 36 | 15.343 | 62.574 | 73.777 | 1.00 | 17.79 | D |
| ATOM | 4326 | C | LEU | D | 36 | 17.038 | 66.074 | 75.854 | 1.00 | 18.18 | D |
| ATOM | 4327 | O | LEU | D | 36 | 17.089 | 66.796 | 76.847 | 1.00 | 17.59 | D |
| ATOM | 4328 | N | PHE | D | 37 | 17.682 | 66.349 | 74.730 | 1.00 | 17.97 | D |
| ATOM | 4329 | CA | PHE | D | 37 | 18.439 | 67.578 | 74.586 | 1.00 | 17.25 | D |
| ATOM | 4330 | CB | PHE | D | 37 | 19.942 | 67.301 | 74.582 | 1.00 | 20.63 | D |
| ATOM | 4331 | CG | PHE | D | 37 | 20.466 | 66.838 | 75.909 | 1.00 | 25.22 | D |
| ATOM | 4332 | CD1 | PHE | D | 37 | 20.392 | 65.498 | 76.274 | 1.00 | 26.75 | D |
| ATOM | 4333 | CD2 | PHE | D | 37 | 20.996 | 67.753 | 76.815 | 1.00 | 26.12 | D |
| ATOM | 4334 | CE1 | PHE | D | 37 | 20.838 | 65.073 | 77.526 | 1.00 | 28.75 | D |
| ATOM | 4335 | CE2 | PHE | D | 37 | 21.444 | 67.341 | 78.069 | 1.00 | 28.86 | D |
| ATOM | 4336 | CZ | PHE | D | 37 | 21.366 | 66.000 | 78.426 | 1.00 | 28.89 | D |
| ATOM | 4337 | C | PHE | D | 37 | 18.013 | 68.225 | 73.282 | 1.00 | 15.52 | D |
| ATOM | 4338 | O | PHE | D | 37 | 17.466 | 67.562 | 72.403 | 1.00 | 14.30 | D |
| ATOM | 4339 | N | HIS | D | 38 | 18.242 | 69.525 | 73.171 | 1.00 | 13.34 | D |
| ATOM | 4340 | CA | HIS | D | 38 | 17.883 | 70.256 | 71.966 | 1.00 | 14.07 | D |
| ATOM | 4341 | CB | HIS | D | 38 | 18.339 | 71.710 | 72.075 | 1.00 | 11.74 | D |
| ATOM | 4342 | CG | HIS | D | 38 | 17.551 | 72.522 | 73.052 | 1.00 | 13.26 | D |
| ATOM | 4343 | CD2 | HIS | D | 38 | 17.918 | 73.145 | 74.197 | 1.00 | 12.15 | D |
| ATOM | 4344 | ND1 | HIS | D | 38 | 16.211 | 72.793 | 72.881 | 1.00 | 12.95 | D |
| ATOM | 4345 | CE1 | HIS | D | 38 | 15.786 | 73.548 | 73.878 | 1.00 | 12.03 | D |
| ATOM | 4346 | NE2 | HIS | D | 38 | 16.802 | 73.776 | 74.690 | 1.00 | 12.60 | D |
| ATOM | 4347 | C | HIS | D | 38 | 18.525 | 69.652 | 70.728 | 1.00 | 13.63 | D |
| ATOM | 4348 | O | HIS | D | 38 | 19.689 | 69.256 | 70.754 | 1.00 | 14.21 | D |
| ATOM | 4349 | N | THR | D | 39 | 17.763 | 69.586 | 69.644 | 1.00 | 12.45 | D |
| ATOM | 4350 | CA | THR | D | 39 | 18.291 | 69.079 | 68.380 | 1.00 | 11.87 | D |
| ATOM | 4351 | CB | THR | D | 39 | 17.155 | 68.763 | 67.396 | 1.00 | 9.67 | D |
| ATOM | 4352 | OG1 | THR | D | 39 | 16.312 | 67.750 | 67.956 | 1.00 | 6.41 | D |
| ATOM | 4353 | CG2 | THR | D | 39 | 17.712 | 68.267 | 66.067 | 1.00 | 10.35 | D |
| ATOM | 4354 | C | THR | D | 39 | 19.122 | 70.244 | 67.833 | 1.00 | 12.09 | D |
| ATOM | 4355 | O | THR | D | 39 | 18.689 | 71.395 | 67.918 | 1.00 | 13.51 | D |
| ATOM | 4356 | N | ILE | D | 40 | 20.310 | 69.966 | 67.295 | 1.00 | 12.95 | D |
| ATOM | 4357 | CA | ILE | D | 40 | 21.154 | 71.035 | 66.758 | 1.00 | 13.05 | D |
| ATOM | 4358 | CB | ILE | D | 40 | 22.536 | 71.087 | 67.455 | 1.00 | 13.45 | D |
| ATOM | 4359 | CG2 | ILE | D | 40 | 22.352 | 71.325 | 68.950 | 1.00 | 13.23 | D |
| ATOM | 4360 | CG1 | ILE | D | 40 | 23.304 | 69.788 | 67.205 | 1.00 | 13.68 | D |
| ATOM | 4361 | CD1 | ILE | D | 40 | 24.761 | 69.852 | 67.635 | 1.00 | 15.23 | D |
| ATOM | 4362 | C | ILE | D | 40 | 21.374 | 70.925 | 65.252 | 1.00 | 13.91 | D |
| ATOM | 4363 | O | ILE | D | 40 | 22.267 | 71.565 | 64.692 | 1.00 | 14.25 | D |
| ATOM | 4364 | N | GLY | D | 41 | 20.556 | 70.106 | 64.604 | 1.00 | 13.40 | D |
| ATOM | 4365 | CA | GLY | D | 41 | 20.659 | 69.945 | 63.170 | 1.00 | 11.86 | D |
| ATOM | 4366 | C | GLY | D | 41 | 19.289 | 69.675 | 62.591 | 1.00 | 12.15 | D |
| ATOM | 4367 | O | GLY | D | 41 | 18.278 | 70.190 | 63.072 | 1.00 | 12.15 | D |
| ATOM | 4368 | N | VAL | D | 42 | 19.263 | 68.861 | 61.546 | 1.00 | 9.58 | D |
| ATOM | 4369 | CA | VAL | D | 42 | 18.031 | 68.487 | 60.887 | 1.00 | 8.78 | D |
| ATOM | 4370 | CB | VAL | D | 42 | 17.903 | 69.182 | 59.511 | 1.00 | 8.11 | D |
| ATOM | 4371 | CG1 | VAL | D | 42 | 16.720 | 68.609 | 58.740 | 1.00 | 8.75 | D |
| ATOM | 4372 | CG2 | VAL | D | 42 | 17.734 | 70.688 | 59.706 | 1.00 | 8.48 | D |
| ATOM | 4373 | C | VAL | D | 42 | 18.073 | 66.981 | 60.691 | 1.00 | 8.18 | D |
| ATOM | 4374 | O | VAL | D | 42 | 19.119 | 66.420 | 60.371 | 1.00 | 9.02 | D |
| ATOM | 4375 | N | GLU | D | 43 | 16.938 | 66.326 | 60.899 | 1.00 | 9.94 | D |
| ATOM | 4376 | CA | GLU | D | 43 | 16.865 | 64.885 | 60.725 | 1.00 | 10.78 | D |
| ATOM | 4377 | CB | GLU | D | 43 | 16.973 | 64.173 | 62.080 | 1.00 | 14.91 | D |
| ATOM | 4378 | CG | GLU | D | 43 | 15.920 | 64.592 | 63.102 | 1.00 | 19.98 | D |
| ATOM | 4379 | CD | GLU | D | 43 | 16.040 | 63.838 | 64.421 | 1.00 | 23.33 | D |
| ATOM | 4380 | OE1 | GLU | D | 43 | 15.266 | 64.142 | 65.358 | 1.00 | 25.77 | D |
| ATOM | 4381 | OE2 | GLU | D | 43 | 16.905 | 62.939 | 64.523 | 1.00 | 24.09 | D |
| ATOM | 4382 | C | GLU | D | 43 | 15.558 | 64.508 | 60.046 | 1.00 | 10.99 | D |
| ATOM | 4383 | O | GLU | D | 43 | 14.488 | 64.984 | 60.430 | 1.00 | 11.62 | D |
| ATOM | 4384 | N | PHE | D | 44 | 15.645 | 63.676 | 59.016 | 1.00 | 9.77 | D |
| ATOM | 4385 | CA | PHE | D | 44 | 14.443 | 63.238 | 58.330 | 1.00 | 8.83 | D |
| ATOM | 4386 | CB | PHE | D | 44 | 14.164 | 64.074 | 57.066 | 1.00 | 8.80 | D |
| ATOM | 4387 | CG | PHE | D | 44 | 15.261 | 64.045 | 56.032 | 1.00 | 9.57 | D |
| ATOM | 4388 | CD1 | PHE | D | 44 | 16.375 | 64.872 | 56.152 | 1.00 | 9.83 | D |
| ATOM | 4389 | CD2 | PHE | D | 44 | 15.149 | 63.228 | 54.906 | 1.00 | 8.36 | D |
| ATOM | 4390 | CE1 | PHE | D | 44 | 17.360 | 64.890 | 55.163 | 1.00 | 9.67 | D |
| ATOM | 4391 | CE2 | PHE | D | 44 | 16.129 | 63.238 | 53.912 | 1.00 | 9.29 | D |
| ATOM | 4392 | CZ | PHE | D | 44 | 17.234 | 64.071 | 54.041 | 1.00 | 8.52 | D |
| ATOM | 4393 | C | PHE | D | 44 | 14.489 | 61.751 | 58.004 | 1.00 | 8.41 | D |
| ATOM | 4394 | O | PHE | D | 44 | 15.537 | 61.108 | 58.078 | 1.00 | 8.04 | D |
| ATOM | 4395 | N | LEU | D | 45 | 13.333 | 61.204 | 57.657 | 1.00 | 8.26 | D |
| ATOM | 4396 | CA | LEU | D | 45 | 13.221 | 59.788 | 57.353 | 1.00 | 8.54 | D |
| ATOM | 4397 | CB | LEU | D | 45 | 13.226 | 58.987 | 58.663 | 1.00 | 9.75 | D |
| ATOM | 4398 | CG | LEU | D | 45 | 12.124 | 59.309 | 59.684 | 1.00 | 10.83 | D |
| ATOM | 4399 | CD1 | LEU | D | 45 | 10.844 | 58.566 | 59.310 | 1.00 | 10.99 | D |
| ATOM | 4400 | CD2 | LEU | D | 45 | 12.564 | 58.887 | 61.077 | 1.00 | 12.24 | D |
| ATOM | 4401 | C | LEU | D | 45 | 11.919 | 59.564 | 56.604 | 1.00 | 7.77 | D |
| ATOM | 4402 | O | LEU | D | 45 | 11.081 | 60.467 | 56.535 | 1.00 | 6.45 | D |
| ATOM | 4403 | N | ASN | D | 46 | 11.757 | 58.364 | 56.047 | 1.00 | 6.80 | D |
| ATOM | 4404 | CA | ASN | D | 46 | 10.547 | 58.008 | 55.304 | 1.00 | 8.38 | D |
| ATOM | 4405 | CB | ASN | D | 46 | 10.902 | 57.336 | 53.966 | 1.00 | 8.86 | D |
| ATOM | 4406 | CG | ASN | D | 46 | 11.401 | 58.313 | 52.914 | 1.00 | 10.64 | D |
| ATOM | 4407 | OD1 | ASN | D | 46 | 11.884 | 57.897 | 51.856 | 1.00 | 15.12 | D |
| ATOM | 4408 | ND2 | ASN | D | 46 | 11.277 | 59.605 | 53.184 | 1.00 | 8.31 | D |
| ATOM | 4409 | C | ASN | D | 46 | 9.690 | 57.028 | 56.100 | 1.00 | 8.19 | D |
| ATOM | 4410 | O | ASN | D | 46 | 10.211 | 56.121 | 56.746 | 1.00 | 9.17 | D |
| ATOM | 4411 | N | LYS | D | 47 | 8.375 | 57.211 | 56.048 | 1.00 | 9.35 | D |
| ATOM | 4412 | CA | LYS | D | 47 | 7.447 | 56.306 | 56.724 | 1.00 | 9.87 | D |
| ATOM | 4413 | CB | LYS | D | 47 | 7.016 | 56.852 | 58.092 | 1.00 | 11.46 | D |
| ATOM | 4414 | CG | LYS | D | 47 | 6.004 | 55.949 | 58.819 | 1.00 | 13.38 | D |
| ATOM | 4415 | CD | LYS | D | 47 | 5.687 | 56.462 | 60.222 | 1.00 | 11.72 | D |
| ATOM | 4416 | CE | LYS | D | 47 | 4.519 | 55.710 | 60.851 | 1.00 | 12.17 | D |
| ATOM | 4417 | NZ | LYS | D | 47 | 4.778 | 54.253 | 60.979 | 1.00 | 14.11 | D |
| ATOM | 4418 | C | LYS | D | 47 | 6.233 | 56.169 | 55.821 | 1.00 | 10.61 | D |
| ATOM | 4419 | O | LYS | D | 47 | 5.613 | 57.166 | 55.455 | 1.00 | 10.81 | D |
| ATOM | 4420 | N | ASP | D | 48 | 5.903 | 54.937 | 55.447 | 1.00 | 9.43 | D |
| ATOM | 4421 | CA | ASP | D | 48 | 4.762 | 54.702 | 54.575 | 1.00 | 11.14 | D |
| ATOM | 4422 | CB | ASP | D | 48 | 4.942 | 53.405 | 53.770 | 1.00 | 13.36 | D |
| ATOM | 4423 | CG | ASP | D | 48 | 6.232 | 53.376 | 52.983 | 1.00 | 17.44 | D |
| ATOM | 4424 | OD1 | ASP | D | 48 | 6.646 | 54.436 | 52.477 | 1.00 | 18.73 | D |
| ATOM | 4425 | OD2 | ASP | D | 48 | 6.829 | 52.283 | 52.862 | 1.00 | 20.68 | D |
| ATOM | 4426 | C | ASP | D | 48 | 3.447 | 54.611 | 55.333 | 1.00 | 11.08 | D |
| ATOM | 4427 | O | ASP | D | 48 | 3.409 | 54.234 | 56.507 | 1.00 | 11.65 | D |
| ATOM | 4428 | N | LEU | D | 49 | 2.368 | 54.968 | 54.645 | 1.00 | 9.86 | D |
| ATOM | 4429 | CA | LEU | D | 49 | 1.030 | 54.888 | 55.214 | 1.00 | 9.93 | D |
| ATOM | 4430 | CB | LEU | D | 49 | 0.775 | 56.021 | 56.217 | 1.00 | 9.97 | D |
| ATOM | 4431 | CG | LEU | D | 49 | 0.528 | 57.447 | 55.709 | 1.00 | 8.78 | D |
| ATOM | 4432 | CD1 | LEU | D | 49 | −0.096 | 58.281 | 56.836 | 1.00 | 9.18 | D |
| ATOM | 4433 | CD2 | LEU | D | 49 | 1.831 | 58.070 | 55.231 | 1.00 | 10.50 | D |
| ATOM | 4434 | C | LEU | D | 49 | 0.012 | 54.973 | 54.088 | 1.00 | 10.21 | D |
| ATOM | 4435 | O | LEU | D | 49 | 0.352 | 55.331 | 52.954 | 1.00 | 10.62 | D |
| ATOM | 4436 | N | GLU | D | 50 | −1.232 | 54.625 | 54.396 | 1.00 | 8.91 | D |
| ATOM | 4437 | CA | GLU | D | 50 | −2.297 | 54.707 | 53.406 | 1.00 | 10.38 | D |
| ATOM | 4438 | CB | GLU | D | 50 | −2.862 | 53.320 | 53.076 | 1.00 | 10.42 | D |
| ATOM | 4439 | CG | GLU | D | 50 | −3.984 | 53.377 | 52.040 | 1.00 | 13.21 | D |
| ATOM | 4440 | CD | GLU | D | 50 | −4.604 | 52.027 | 51.727 | 1.00 | 15.06 | D |
| ATOM | 4441 | OE1 | GLU | D | 50 | −4.457 | 51.081 | 52.533 | 1.00 | 15.80 | D |
| ATOM | 4442 | OE2 | GLU | D | 50 | −5.264 | 51.920 | 50.673 | 1.00 | 14.86 | D |
| ATOM | 4443 | C | GLU | D | 50 | −3.402 | 55.592 | 53.960 | 1.00 | 9.48 | D |
| ATOM | 4444 | O | GLU | D | 50 | −3.828 | 55.429 | 55.106 | 1.00 | 10.66 | D |
| ATOM | 4445 | N | VAL | D | 51 | −3.846 | 56.546 | 53.149 | 1.00 | 8.79 | D |
| ATOM | 4446 | CA | VAL | D | 51 | −4.912 | 57.461 | 53.536 | 1.00 | 8.25 | D |
| ATOM | 4447 | CB | VAL | D | 51 | −4.363 | 58.855 | 53.913 | 1.00 | 9.74 | D |
| ATOM | 4448 | CG1 | VAL | D | 51 | −5.519 | 59.803 | 54.201 | 1.00 | 10.33 | D |
| ATOM | 4449 | CG2 | VAL | D | 51 | −3.442 | 58.753 | 55.113 | 1.00 | 10.29 | D |
| ATOM | 4450 | C | VAL | D | 51 | −5.820 | 57.617 | 52.326 | 1.00 | 8.01 | D |
| ATOM | 4451 | O | VAL | D | 51 | −5.340 | 57.859 | 51.220 | 1.00 | 8.81 | D |
| ATOM | 4452 | N | ASP | D | 52 | −7.126 | 57.485 | 52.534 | 1.00 | 7.55 | D |
| ATOM | 4453 | CA | ASP | D | 52 | −8.085 | 57.593 | 51.438 | 1.00 | 9.43 | D |
| ATOM | 4454 | CB | ASP | D | 52 | −8.214 | 59.044 | 50.953 | 1.00 | 11.02 | D |
| ATOM | 4455 | CG | ASP | D | 52 | −8.825 | 59.960 | 51.991 | 1.00 | 13.94 | D |
| ATOM | 4456 | OD1 | ASP | D | 52 | −9.586 | 59.474 | 52.853 | 1.00 | 14.42 | D |
| ATOM | 4457 | OD2 | ASP | D | 52 | −8.553 | 61.178 | 51.934 | 1.00 | 17.02 | D |
| ATOM | 4458 | C | ASP | D | 52 | −7.701 | 56.711 | 50.248 | 1.00 | 9.11 | D |
| ATOM | 4459 | O | ASP | D | 52 | −7.906 | 57.094 | 49.099 | 1.00 | 11.32 | D |
| ATOM | 4460 | N | GLY | D | 53 | −7.132 | 55.541 | 50.523 | 1.00 | 8.51 | D |
| ATOM | 4461 | CA | GLY | D | 53 | −6.755 | 54.632 | 49.454 | 1.00 | 7.71 | D |
| ATOM | 4462 | C | GLY | D | 53 | −5.506 | 55.003 | 48.680 | 1.00 | 10.00 | D |
| ATOM | 4463 | O | GLY | D | 53 | −5.198 | 54.386 | 47.658 | 1.00 | 11.45 | D |
| ATOM | 4464 | N | HIS | D | 54 | −4.796 | 56.015 | 49.159 | 1.00 | 9.72 | D |
| ATOM | 4465 | CA | HIS | D | 54 | −3.563 | 56.463 | 48.527 | 1.00 | 10.86 | D |
| ATOM | 4466 | CB | HIS | D | 54 | −3.500 | 57.992 | 48.515 | 1.00 | 12.06 | D |
| ATOM | 4467 | CG | HIS | D | 54 | −4.428 | 58.631 | 47.532 | 1.00 | 12.94 | D |
| ATOM | 4468 | CD2 | HIS | D | 54 | −5.748 | 58.919 | 47.611 | 1.00 | 13.27 | D |
| ATOM | 4469 | ND1 | HIS | D | 54 | −4.018 | 59.044 | 46.282 | 1.00 | 12.85 | D |
| ATOM | 4470 | CE1 | HIS | D | 54 | −5.047 | 59.561 | 45.634 | 1.00 | 15.45 | D |
| ATOM | 4471 | NE2 | HIS | D | 54 | −6.108 | 59.497 | 46.418 | 1.00 | 14.04 | D |
| ATOM | 4472 | C | HIS | D | 54 | −2.390 | 55.924 | 49.329 | 1.00 | 10.66 | D |
| ATOM | 4473 | O | HIS | D | 54 | −2.286 | 56.186 | 50.527 | 1.00 | 11.16 | D |
| ATOM | 4474 | N | PHE | D | 55 | −1.523 | 55.155 | 48.679 | 1.00 | 11.80 | D |
| ATOM | 4475 | CA | PHE | D | 55 | −0.349 | 54.615 | 49.351 | 1.00 | 13.18 | D |
| ATOM | 4476 | CB | PHE | D | 55 | 0.075 | 53.297 | 48.703 | 1.00 | 14.84 | D |
| ATOM | 4477 | CG | PHE | D | 55 | −0.858 | 52.158 | 49.005 | 1.00 | 18.45 | D |
| ATOM | 4478 | CD1 | PHE | D | 55 | −0.801 | 51.506 | 50.233 | 1.00 | 20.12 | D |
| ATOM | 4479 | CD2 | PHE | D | 55 | −1.826 | 51.770 | 48.084 | 1.00 | 19.47 | D |
| ATOM | 4480 | CE1 | PHE | D | 55 | −1.699 | 50.481 | 50.545 | 1.00 | 20.25 | D |
| ATOM | 4481 | CE2 | PHE | D | 55 | −2.730 | 50.747 | 48.385 | 1.00 | 20.68 | D |
| ATOM | 4482 | CZ | PHE | D | 55 | −2.665 | 50.104 | 49.618 | 1.00 | 19.68 | D |
| ATOM | 4483 | C | PHE | D | 55 | 0.729 | 55.676 | 49.208 | 1.00 | 13.98 | D |
| ATOM | 4484 | O | PHE | D | 55 | 1.253 | 55.911 | 48.118 | 1.00 | 13.79 | D |
| ATOM | 4485 | N | VAL | D | 56 | 1.038 | 56.330 | 50.321 | 1.00 | 13.18 | D |
| ATOM | 4486 | CA | VAL | D | 56 | 2.009 | 57.408 | 50.324 | 1.00 | 13.81 | D |
| ATOM | 4487 | CB | VAL | D | 56 | 1.332 | 58.719 | 50.799 | 1.00 | 16.90 | D |
| ATOM | 4488 | CG1 | VAL | D | 56 | 2.347 | 59.848 | 50.875 | 1.00 | 20.14 | D |
| ATOM | 4489 | CG2 | VAL | D | 56 | 0.196 | 59.085 | 49.851 | 1.00 | 16.61 | D |
| ATOM | 4490 | C | VAL | D | 56 | 3.222 | 57.139 | 51.198 | 1.00 | 12.35 | D |
| ATOM | 4491 | O | VAL | D | 56 | 3.185 | 56.320 | 52.112 | 1.00 | 12.55 | D |
| ATOM | 4492 | N | THR | D | 57 | 4.314 | 57.819 | 50.883 | 1.00 | 11.58 | D |
| ATOM | 4493 | CA | THR | D | 57 | 5.531 | 57.711 | 51.667 | 1.00 | 9.87 | D |
| ATOM | 4494 | CB | THR | D | 57 | 6.764 | 57.377 | 50.800 | 1.00 | 10.34 | D |
| ATOM | 4495 | OG1 | THR | D | 57 | 6.651 | 56.038 | 50.300 | 1.00 | 11.81 | D |
| ATOM | 4496 | CG2 | THR | D | 57 | 8.043 | 57.491 | 51.626 | 1.00 | 11.00 | D |
| ATOM | 4497 | C | THR | D | 57 | 5.696 | 59.097 | 52.256 | 1.00 | 9.93 | D |
| ATOM | 4498 | O | THR | D | 57 | 5.928 | 60.064 | 51.530 | 1.00 | 12.02 | D |
| ATOM | 4499 | N | MET | D | 58 | 5.531 | 59.206 | 53.566 | 1.00 | 9.23 | D |
| ATOM | 4500 | CA | MET | D | 58 | 5.681 | 60.497 | 54.213 | 1.00 | 8.90 | D |
| ATOM | 4501 | CB | MET | D | 58 | 4.766 | 60.613 | 55.431 | 1.00 | 9.92 | D |
| ATOM | 4502 | CG | MET | D | 58 | 4.903 | 61.950 | 56.142 | 1.00 | 13.36 | D |
| ATOM | 4503 | SD | MET | D | 58 | 3.990 | 62.040 | 57.695 | 1.00 | 16.16 | D |
| ATOM | 4504 | CE | MET | D | 58 | 2.323 | 62.126 | 57.100 | 1.00 | 16.18 | D |
| ATOM | 4505 | C | MET | D | 58 | 7.119 | 60.684 | 54.660 | 1.00 | 9.57 | D |
| ATOM | 4506 | O | MET | D | 58 | 7.670 | 59.851 | 55.378 | 1.00 | 9.41 | D |
| ATOM | 4507 | N | GLN | D | 59 | 7.733 | 61.774 | 54.220 | 1.00 | 8.34 | D |
| ATOM | 4508 | CA | GLN | D | 59 | 9.099 | 62.062 | 54.617 | 1.00 | 8.09 | D |
| ATOM | 4509 | CB | GLN | D | 59 | 9.916 | 62.542 | 53.419 | 1.00 | 8.26 | D |
| ATOM | 4510 | CG | GLN | D | 59 | 11.387 | 62.759 | 53.721 | 1.00 | 8.89 | D |
| ATOM | 4511 | CD | GLN | D | 59 | 12.204 | 62.891 | 52.459 | 1.00 | 9.52 | D |
| ATOM | 4512 | OE1 | GLN | D | 59 | 12.581 | 61.893 | 51.836 | 1.00 | 11.31 | D |
| ATOM | 4513 | NE2 | GLN | D | 59 | 12.464 | 64.122 | 52.060 | 1.00 | 6.91 | D |
| ATOM | 4514 | C | GLN | D | 59 | 8.992 | 63.148 | 55.676 | 1.00 | 8.42 | D |
| ATOM | 4515 | O | GLN | D | 59 | 8.703 | 64.308 | 55.373 | 1.00 | 7.47 | D |
| ATOM | 4516 | N | ILE | D | 60 | 9.188 | 62.743 | 56.926 | 1.00 | 8.12 | D |
| ATOM | 4517 | CA | ILE | D | 60 | 9.095 | 63.654 | 58.059 | 1.00 | 8.42 | D |
| ATOM | 4518 | CB | ILE | D | 60 | 8.643 | 62.911 | 59.334 | 1.00 | 8.25 | D |
| ATOM | 4519 | CG2 | ILE | D | 60 | 8.421 | 63.914 | 60.473 | 1.00 | 7.49 | D |
| ATOM | 4520 | CG1 | ILE | D | 60 | 7.336 | 62.163 | 59.056 | 1.00 | 7.25 | D |
| ATOM | 4521 | CD1 | ILE | D | 60 | 6.924 | 61.202 | 60.154 | 1.00 | 8.51 | D |
| ATOM | 4522 | C | ILE | D | 60 | 10.422 | 64.332 | 58.343 | 1.00 | 8.28 | D |
| ATOM | 4523 | O | ILE | D | 60 | 11.443 | 63.664 | 58.557 | 1.00 | 8.84 | D |
| ATOM | 4524 | N | TRP | D | 61 | 10.386 | 65.662 | 58.346 | 1.00 | 8.66 | D |
| ATOM | 4525 | CA | TRP | D | 61 | 11.554 | 66.491 | 58.612 | 1.00 | 8.79 | D |
| ATOM | 4526 | CB | TRP | D | 61 | 11.687 | 67.588 | 57.551 | 1.00 | 8.39 | D |
| ATOM | 4527 | CG | TRP | D | 61 | 12.207 | 67.100 | 56.244 | 1.00 | 6.63 | D |
| ATOM | 4528 | CD2 | TRP | D | 61 | 13.424 | 67.501 | 55.606 | 1.00 | 7.95 | D |
| ATOM | 4529 | CE2 | TRP | D | 61 | 13.518 | 66.774 | 54.399 | 1.00 | 8.43 | D |
| ATOM | 4530 | CE3 | TRP | D | 61 | 14.445 | 68.403 | 55.936 | 1.00 | 7.39 | D |
| ATOM | 4531 | CD1 | TRP | D | 61 | 11.629 | 66.177 | 55.427 | 1.00 | 8.37 | D |
| ATOM | 4532 | NE1 | TRP | D | 61 | 12.409 | 65.975 | 54.315 | 1.00 | 6.27 | D |
| ATOM | 4533 | CZ2 | TRP | D | 61 | 14.596 | 66.920 | 53.516 | 1.00 | 7.80 | D |
| ATOM | 4534 | CZ3 | TRP | D | 61 | 15.520 | 68.549 | 55.056 | 1.00 | 9.86 | D |
| ATOM | 4535 | CH2 | TRP | D | 61 | 15.584 | 67.809 | 53.860 | 1.00 | 8.80 | D |
| ATOM | 4536 | C | TRP | D | 61 | 11.456 | 67.150 | 59.984 | 1.00 | 10.00 | D |
| ATOM | 4537 | O | TRP | D | 61 | 10.537 | 67.929 | 60.246 | 1.00 | 10.25 | D |
| ATOM | 4538 | N | ASP | D | 62 | 12.405 | 66.826 | 60.852 | 1.00 | 10.41 | D |
| ATOM | 4539 | CA | ASP | D | 62 | 12.457 | 67.399 | 62.191 | 1.00 | 12.54 | D |
| ATOM | 4540 | CB | ASP | D | 62 | 12.702 | 66.297 | 63.225 | 1.00 | 11.14 | D |
| ATOM | 4541 | CG | ASP | D | 62 | 12.912 | 66.840 | 64.625 | 1.00 | 13.67 | D |
| ATOM | 4542 | OD1 | ASP | D | 62 | 12.355 | 67.912 | 64.954 | 1.00 | 11.80 | D |
| ATOM | 4543 | OD2 | ASP | D | 62 | 13.627 | 66.175 | 65.404 | 1.00 | 14.45 | D |
| ATOM | 4544 | C | ASP | D | 62 | 13.595 | 68.407 | 62.200 | 1.00 | 13.80 | D |
| ATOM | 4545 | O | ASP | D | 62 | 14.736 | 68.071 | 61.881 | 1.00 | 15.87 | D |
| ATOM | 4546 | N | THR | D | 63 | 13.283 | 69.646 | 62.553 | 1.00 | 16.13 | D |
| ATOM | 4547 | CA | THR | D | 63 | 14.293 | 70.692 | 62.575 | 1.00 | 17.30 | D |
| ATOM | 4548 | CB | THR | D | 63 | 13.901 | 71.855 | 61.632 | 1.00 | 18.61 | D |
| ATOM | 4549 | OG1 | THR | D | 63 | 12.744 | 72.524 | 62.151 | 1.00 | 19.82 | D |
| ATOM | 4550 | CG2 | THR | D | 63 | 13.580 | 71.325 | 60.239 | 1.00 | 19.81 | D |
| ATOM | 4551 | C | THR | D | 63 | 14.525 | 71.264 | 63.970 | 1.00 | 15.83 | D |
| ATOM | 4552 | O | THR | D | 63 | 13.660 | 71.184 | 64.847 | 1.00 | 16.31 | D |
| ATOM | 4553 | N | ALA | D | 64 | 15.706 | 71.838 | 64.170 | 1.00 | 15.74 | D |
| ATOM | 4554 | CA | ALA | D | 64 | 16.048 | 72.453 | 65.445 | 1.00 | 14.86 | D |
| ATOM | 4555 | CB | ALA | D | 64 | 17.524 | 72.810 | 65.467 | 1.00 | 15.62 | D |
| ATOM | 4556 | C | ALA | D | 64 | 15.192 | 73.713 | 65.557 | 1.00 | 14.13 | D |
| ATOM | 4557 | O | ALA | D | 64 | 15.214 | 74.557 | 64.665 | 1.00 | 15.57 | D |
| ATOM | 4558 | N | GLY | D | 65 | 14.444 | 73.838 | 66.649 | 1.00 | 14.71 | D |
| ATOM | 4559 | CA | GLY | D | 65 | 13.574 | 74.990 | 66.819 | 1.00 | 14.01 | D |
| ATOM | 4560 | C | GLY | D | 65 | 14.197 | 76.291 | 67.287 | 1.00 | 14.57 | D |
| ATOM | 4561 | O | GLY | D | 65 | 13.629 | 77.362 | 67.065 | 1.00 | 12.29 | D |
| ATOM | 4562 | N | GLN | D | 66 | 15.357 | 76.215 | 67.932 | 1.00 | 14.17 | D |
| ATOM | 4563 | CA | GLN | D | 66 | 16.016 | 77.416 | 68.426 | 1.00 | 13.36 | D |
| ATOM | 4564 | CB | GLN | D | 66 | 17.240 | 77.033 | 69.254 | 1.00 | 15.50 | D |
| ATOM | 4565 | CG | GLN | D | 66 | 16.865 | 76.346 | 70.559 | 1.00 | 15.47 | D |
| ATOM | 4566 | CD | GLN | D | 66 | 18.066 | 75.868 | 71.351 | 1.00 | 18.18 | D |
| ATOM | 4567 | OE1 | GLN | D | 66 | 18.702 | 74.870 | 71.001 | 1.00 | 17.01 | D |
| ATOM | 4568 | NE2 | GLN | D | 66 | 18.387 | 76.582 | 72.423 | 1.00 | 18.15 | D |
| ATOM | 4569 | C | GLN | D | 66 | 16.391 | 78.394 | 67.315 | 1.00 | 14.79 | D |
| ATOM | 4570 | O | GLN | D | 66 | 16.764 | 77.996 | 66.212 | 1.00 | 12.77 | D |
| ATOM | 4571 | N | GLU | D | 67 | 16.285 | 79.682 | 67.630 | 1.00 | 14.60 | D |
| ATOM | 4572 | CA | GLU | D | 67 | 16.568 | 80.757 | 66.684 | 1.00 | 16.68 | D |
| ATOM | 4573 | CB | GLU | D | 67 | 16.430 | 82.114 | 67.387 | 1.00 | 16.75 | D |
| ATOM | 4574 | CG | GLU | D | 67 | 15.016 | 82.444 | 67.847 | 0.00 | 18.05 | D |
| ATOM | 4575 | CD | GLU | D | 67 | 14.485 | 81.457 | 68.868 | 0.00 | 18.51 | D |
| ATOM | 4576 | OE1 | GLU | D | 67 | 15.104 | 81.321 | 69.944 | 0.00 | 18.83 | D |
| ATOM | 4577 | OE2 | GLU | D | 67 | 13.448 | 80.817 | 68.594 | 0.00 | 18.83 | D |
| ATOM | 4578 | C | GLU | D | 67 | 17.918 | 80.700 | 65.973 | 1.00 | 17.30 | D |
| ATOM | 4579 | O | GLU | D | 67 | 18.015 | 81.053 | 64.796 | 1.00 | 17.72 | D |
| ATOM | 4580 | N | ARG | D | 68 | 18.961 | 80.260 | 66.671 | 1.00 | 18.89 | D |
| ATOM | 4581 | CA | ARG | D | 68 | 20.284 | 80.216 | 66.057 | 1.00 | 20.39 | D |
| ATOM | 4582 | CB | ARG | D | 68 | 21.366 | 79.979 | 67.117 | 1.00 | 22.50 | D |
| ATOM | 4583 | CG | ARG | D | 68 | 21.352 | 78.608 | 67.774 | 1.00 | 23.96 | D |
| ATOM | 4584 | CD | ARG | D | 68 | 22.637 | 78.409 | 68.572 | 1.00 | 27.33 | D |
| ATOM | 4585 | NE | ARG | D | 68 | 22.699 | 77.114 | 69.240 | 1.00 | 29.90 | D |
| ATOM | 4586 | CZ | ARG | D | 68 | 21.903 | 76.747 | 70.239 | 1.00 | 30.19 | D |
| ATOM | 4587 | NH1 | ARG | D | 68 | 20.974 | 77.579 | 70.693 | 1.00 | 30.66 | D |
| ATOM | 4588 | NH2 | ARG | D | 68 | 22.041 | 75.548 | 70.787 | 1.00 | 31.54 | D |
| ATOM | 4589 | C | ARG | D | 68 | 20.432 | 79.190 | 64.937 | 1.00 | 20.03 | D |
| ATOM | 4590 | O | ARG | D | 68 | 21.454 | 79.166 | 64.251 | 1.00 | 20.29 | D |
| ATOM | 4591 | N | PHE | D | 69 | 19.418 | 78.352 | 64.739 | 1.00 | 19.43 | D |
| ATOM | 4592 | CA | PHE | D | 69 | 19.478 | 77.343 | 63.687 | 1.00 | 18.89 | D |
| ATOM | 4593 | CB | PHE | D | 69 | 19.039 | 75.980 | 64.228 | 1.00 | 20.15 | D |
| ATOM | 4594 | CG | PHE | D | 69 | 19.911 | 75.463 | 65.331 | 1.00 | 19.35 | D |
| ATOM | 4595 | CD1 | PHE | D | 69 | 19.465 | 75.463 | 66.647 | 1.00 | 19.94 | D |
| ATOM | 4596 | CD2 | PHE | D | 69 | 21.191 | 74.997 | 65.058 | 1.00 | 20.14 | D |
| ATOM | 4597 | CE1 | PHE | D | 69 | 20.282 | 75.008 | 67.676 | 1.00 | 19.38 | D |
| ATOM | 4598 | CE2 | PHE | D | 69 | 22.017 | 74.539 | 66.081 | 1.00 | 21.49 | D |
| ATOM | 4599 | CZ | PHE | D | 69 | 21.560 | 74.545 | 67.393 | 1.00 | 21.21 | D |
| ATOM | 4600 | C | PHE | D | 69 | 18.618 | 77.713 | 62.487 | 1.00 | 18.76 | D |
| ATOM | 4601 | O | PHE | D | 69 | 18.350 | 76.880 | 61.623 | 1.00 | 18.30 | D |
| ATOM | 4602 | N | ARG | D | 70 | 18.197 | 78.970 | 62.436 | 1.00 | 18.76 | D |
| ATOM | 4603 | CA | ARG | D | 70 | 17.358 | 79.456 | 61.350 | 1.00 | 19.13 | D |
| ATOM | 4604 | CB | ARG | D | 70 | 16.947 | 80.906 | 61.626 | 1.00 | 19.75 | D |
| ATOM | 4605 | CG | ARG | D | 70 | 15.937 | 81.478 | 60.647 | 1.00 | 22.17 | D |
| ATOM | 4606 | CD | ARG | D | 70 | 15.410 | 82.816 | 61.153 | 1.00 | 26.11 | D |
| ATOM | 4607 | NE | ARG | D | 70 | 14.923 | 82.700 | 62.527 | 1.00 | 30.38 | D |
| ATOM | 4608 | CZ | ARG | D | 70 | 14.389 | 83.696 | 63.229 | 1.00 | 32.63 | D |
| ATOM | 4609 | NH1 | ARG | D | 70 | 14.264 | 84.903 | 62.689 | 1.00 | 34.24 | D |
| ATOM | 4610 | NH2 | ARG | D | 70 | 13.984 | 83.485 | 64.475 | 1.00 | 31.71 | D |
| ATOM | 4611 | C | ARG | D | 70 | 18.034 | 79.365 | 59.984 | 1.00 | 18.96 | D |
| ATOM | 4612 | O | ARG | D | 70 | 17.455 | 78.835 | 59.035 | 1.00 | 18.88 | D |
| ATOM | 4613 | N | SER | D | 71 | 19.261 | 79.871 | 59.885 | 1.00 | 18.90 | D |
| ATOM | 4614 | CA | SER | D | 71 | 19.981 | 79.857 | 58.614 | 1.00 | 17.24 | D |
| ATOM | 4615 | CB | SER | D | 71 | 21.303 | 80.617 | 58.742 | 1.00 | 17.88 | D |
| ATOM | 4616 | OG | SER | D | 71 | 22.189 | 79.966 | 59.634 | 1.00 | 21.61 | D |
| ATOM | 4617 | C | SER | D | 71 | 20.243 | 78.448 | 58.092 | 1.00 | 15.98 | D |
| ATOM | 4618 | O | SER | D | 71 | 20.360 | 78.240 | 56.882 | 1.00 | 15.60 | D |
| ATOM | 4619 | N | LEU | D | 72 | 20.327 | 77.483 | 59.002 | 1.00 | 13.79 | D |
| ATOM | 4620 | CA | LEU | D | 72 | 20.571 | 76.091 | 58.620 | 1.00 | 13.32 | D |
| ATOM | 4621 | CB | LEU | D | 72 | 21.041 | 75.287 | 59.837 | 1.00 | 15.72 | D |
| ATOM | 4622 | CG | LEU | D | 72 | 20.935 | 73.760 | 59.743 | 1.00 | 16.30 | D |
| ATOM | 4623 | CD1 | LEU | D | 72 | 21.905 | 73.226 | 58.700 | 1.00 | 18.55 | D |
| ATOM | 4624 | CD2 | LEU | D | 72 | 21.227 | 73.147 | 61.099 | 1.00 | 17.35 | D |
| ATOM | 4625 | C | LEU | D | 72 | 19.346 | 75.396 | 58.031 | 1.00 | 13.35 | D |
| ATOM | 4626 | O | LEU | D | 72 | 19.450 | 74.665 | 57.044 | 1.00 | 13.48 | D |
| ATOM | 4627 | N | ARG | D | 73 | 18.188 | 75.629 | 58.639 | 1.00 | 12.32 | D |
| ATOM | 4628 | CA | ARG | D | 73 | 16.951 | 74.976 | 58.215 | 1.00 | 12.54 | D |
| ATOM | 4629 | CB | ARG | D | 73 | 16.061 | 74.733 | 59.441 | 1.00 | 11.84 | D |
| ATOM | 4630 | CG | ARG | D | 73 | 15.612 | 76.019 | 60.120 | 1.00 | 12.18 | D |
| ATOM | 4631 | CD | ARG | D | 73 | 14.650 | 75.773 | 61.280 | 1.00 | 11.27 | D |
| ATOM | 4632 | NE | ARG | D | 73 | 14.058 | 77.040 | 61.700 | 1.00 | 12.08 | D |
| ATOM | 4633 | CZ | ARG | D | 73 | 14.334 | 77.681 | 62.830 | 1.00 | 13.66 | D |
| ATOM | 4634 | NH1 | ARG | D | 73 | 15.199 | 77.179 | 63.703 | 1.00 | 13.31 | D |
| ATOM | 4635 | NH2 | ARG | D | 73 | 13.758 | 78.851 | 63.070 | 1.00 | 15.46 | D |
| ATOM | 4636 | C | ARG | D | 73 | 16.118 | 75.662 | 57.136 | 1.00 | 11.98 | D |
| ATOM | 4637 | O | ARG | D | 73 | 15.480 | 74.992 | 56.327 | 1.00 | 11.92 | D |
| ATOM | 4638 | N | THR | D | 74 | 16.116 | 76.988 | 57.107 | 1.00 | 14.08 | D |
| ATOM | 4639 | CA | THR | D | 74 | 15.304 | 77.690 | 56.121 | 1.00 | 14.01 | D |
| ATOM | 4640 | CB | THR | D | 74 | 15.425 | 79.227 | 56.276 | 1.00 | 13.88 | D |
| ATOM | 4641 | OG1 | THR | D | 74 | 16.803 | 79.609 | 56.353 | 1.00 | 13.94 | D |
| ATOM | 4642 | CG2 | THR | D | 74 | 14.701 | 79.684 | 57.538 | 1.00 | 14.62 | D |
| ATOM | 4643 | C | THR | D | 74 | 15.526 | 77.298 | 54.658 | 1.00 | 14.61 | D |
| ATOM | 4644 | O | THR | D | 74 | 14.580 | 77.280 | 53.873 | 1.00 | 15.90 | D |
| ATOM | 4645 | N | PRO | D | 75 | 16.767 | 76.972 | 54.265 | 1.00 | 14.28 | D |
| ATOM | 4646 | CD | PRO | D | 75 | 18.072 | 77.075 | 54.937 | 1.00 | 14.73 | D |
| ATOM | 4647 | CA | PRO | D | 75 | 16.923 | 76.602 | 52.855 | 1.00 | 15.25 | D |
| ATOM | 4648 | CB | PRO | D | 75 | 18.444 | 76.553 | 52.669 | 1.00 | 14.18 | D |
| ATOM | 4649 | CG | PRO | D | 75 | 18.956 | 76.255 | 54.036 | 1.00 | 18.05 | D |
| ATOM | 4650 | C | PRO | D | 75 | 16.234 | 75.282 | 52.488 | 1.00 | 14.44 | D |
| ATOM | 4651 | O | PRO | D | 75 | 16.115 | 74.939 | 51.310 | 1.00 | 14.42 | D |
| ATOM | 4652 | N | PHE | D | 76 | 15.764 | 74.557 | 53.497 | 1.00 | 12.81 | D |
| ATOM | 4653 | CA | PHE | D | 76 | 15.103 | 73.277 | 53.267 | 1.00 | 14.04 | D |
| ATOM | 4654 | CB | PHE | D | 76 | 15.697 | 72.230 | 54.212 | 1.00 | 13.34 | D |
| ATOM | 4655 | CG | PHE | D | 76 | 17.156 | 71.998 | 53.978 | 1.00 | 15.07 | D |
| ATOM | 4656 | CD1 | PHE | D | 76 | 17.582 | 71.194 | 52.925 | 1.00 | 13.89 | D |
| ATOM | 4657 | CD2 | PHE | D | 76 | 18.108 | 72.661 | 54.744 | 1.00 | 15.43 | D |
| ATOM | 4658 | CE1 | PHE | D | 76 | 18.933 | 71.063 | 52.633 | 1.00 | 15.03 | D |
| ATOM | 4659 | CE2 | PHE | D | 76 | 19.462 | 72.537 | 54.460 | 1.00 | 15.49 | D |
| ATOM | 4660 | CZ | PHE | D | 76 | 19.875 | 71.739 | 53.403 | 1.00 | 15.20 | D |
| ATOM | 4661 | C | PHE | D | 76 | 13.583 | 73.322 | 53.388 | 1.00 | 15.55 | D |
| ATOM | 4662 | O | PHE | D | 76 | 12.917 | 72.289 | 53.299 | 1.00 | 16.41 | D |
| ATOM | 4663 | N | TYR | D | 77 | 13.033 | 74.516 | 53.590 | 1.00 | 15.66 | D |
| ATOM | 4664 | CA | TYR | D | 77 | 11.588 | 74.660 | 53.689 | 1.00 | 15.34 | D |
| ATOM | 4665 | CB | TYR | D | 77 | 11.209 | 76.082 | 54.115 | 1.00 | 13.77 | D |
| ATOM | 4666 | CG | TYR | D | 77 | 11.469 | 76.425 | 55.573 | 1.00 | 11.96 | D |
| ATOM | 4667 | CD1 | TYR | D | 77 | 12.049 | 75.506 | 56.450 | 1.00 | 11.40 | D |
| ATOM | 4668 | CE1 | TYR | D | 77 | 12.272 | 75.833 | 57.792 | 1.00 | 10.64 | D |
| ATOM | 4669 | CD2 | TYR | D | 77 | 11.121 | 77.678 | 56.074 | 1.00 | 11.32 | D |
| ATOM | 4670 | CE2 | TYR | D | 77 | 11.336 | 78.011 | 57.404 | 1.00 | 12.46 | D |
| ATOM | 4671 | CZ | TYR | D | 77 | 11.910 | 77.090 | 58.259 | 1.00 | 12.16 | D |
| ATOM | 4672 | OH | TYR | D | 77 | 12.105 | 77.435 | 59.581 | 1.00 | 11.68 | D |
| ATOM | 4673 | C | TYR | D | 77 | 11.000 | 74.367 | 52.311 | 1.00 | 15.81 | D |
| ATOM | 4674 | O | TYR | D | 77 | 9.968 | 73.710 | 52.192 | 1.00 | 15.64 | D |
| ATOM | 4675 | N | ARG | D | 78 | 11.669 | 74.857 | 51.270 | 1.00 | 16.34 | D |
| ATOM | 4676 | CA | ARG | D | 78 | 11.214 | 74.646 | 49.900 | 1.00 | 17.28 | D |
| ATOM | 4677 | CB | ARG | D | 78 | 12.244 | 75.206 | 48.915 | 1.00 | 22.42 | D |
| ATOM | 4678 | CG | ARG | D | 78 | 12.530 | 76.682 | 49.122 | 1.00 | 27.97 | D |
| ATOM | 4679 | CD | ARG | D | 78 | 13.753 | 77.148 | 48.340 | 1.00 | 32.95 | D |
| ATOM | 4680 | NE | ARG | D | 78 | 14.101 | 78.530 | 48.665 | 1.00 | 36.46 | D |
| ATOM | 4681 | CZ | ARG | D | 78 | 14.415 | 78.954 | 49.887 | 1.00 | 38.72 | D |
| ATOM | 4682 | NH1 | ARG | D | 78 | 14.427 | 78.101 | 50.906 | 1.00 | 40.39 | D |
| ATOM | 4683 | NH2 | ARG | D | 78 | 14.709 | 80.233 | 50.095 | 1.00 | 38.62 | D |
| ATOM | 4684 | C | ARG | D | 78 | 10.988 | 73.162 | 49.624 | 1.00 | 16.18 | D |
| ATOM | 4685 | O | ARG | D | 78 | 11.772 | 72.309 | 50.047 | 1.00 | 14.52 | D |
| ATOM | 4686 | N | GLY | D | 79 | 9.903 | 72.860 | 48.920 | 1.00 | 15.61 | D |
| ATOM | 4687 | CA | GLY | D | 79 | 9.595 | 71.481 | 48.598 | 1.00 | 13.56 | D |
| ATOM | 4688 | C | GLY | D | 79 | 8.668 | 70.808 | 49.594 | 1.00 | 13.24 | D |
| ATOM | 4689 | O | GLY | D | 79 | 8.170 | 69.716 | 49.331 | 1.00 | 12.14 | D |
| ATOM | 4690 | N | SER | D | 80 | 8.437 | 71.441 | 50.740 | 1.00 | 12.85 | D |
| ATOM | 4691 | CA | SER | D | 80 | 7.548 | 70.864 | 51.743 | 1.00 | 12.05 | D |
| ATOM | 4692 | CB | SER | D | 80 | 7.582 | 71.683 | 53.037 | 1.00 | 11.18 | D |
| ATOM | 4693 | OG | SER | D | 80 | 8.881 | 71.706 | 53.598 | 1.00 | 11.89 | D |
| ATOM | 4694 | C | SER | D | 80 | 6.130 | 70.857 | 51.189 | 1.00 | 12.57 | D |
| ATOM | 4695 | O | SER | D | 80 | 5.708 | 71.816 | 50.545 | 1.00 | 13.21 | D |
| ATOM | 4696 | N | ASP | D | 81 | 5.399 | 69.776 | 51.437 | 1.00 | 11.39 | D |
| ATOM | 4697 | CA | ASP | D | 81 | 4.027 | 69.669 | 50.970 | 1.00 | 11.69 | D |
| ATOM | 4698 | CB | ASP | D | 81 | 3.747 | 68.249 | 50.492 | 1.00 | 13.14 | D |
| ATOM | 4699 | CG | ASP | D | 81 | 4.588 | 67.873 | 49.288 | 1.00 | 16.51 | D |
| ATOM | 4700 | OD1 | ASP | D | 81 | 4.451 | 68.543 | 48.245 | 1.00 | 17.64 | D |
| ATOM | 4701 | OD2 | ASP | D | 81 | 5.387 | 66.918 | 49.385 | 1.00 | 16.27 | D |
| ATOM | 4702 | C | ASP | D | 81 | 3.055 | 70.066 | 52.074 | 1.00 | 11.19 | D |
| ATOM | 4703 | O | ASP | D | 81 | 1.943 | 70.504 | 51.802 | 1.00 | 10.78 | D |
| ATOM | 4704 | N | CYS | D | 82 | 3.490 | 69.919 | 53.320 | 1.00 | 11.01 | D |
| ATOM | 4705 | CA | CYS | D | 82 | 2.671 | 70.287 | 54.469 | 1.00 | 11.37 | D |
| ATOM | 4706 | CB | CYS | D | 82 | 1.786 | 69.117 | 54.899 | 1.00 | 10.98 | D |
| ATOM | 4707 | SG | CYS | D | 82 | 0.633 | 69.547 | 56.218 | 1.00 | 16.35 | D |
| ATOM | 4708 | C | CYS | D | 82 | 3.573 | 70.696 | 55.625 | 1.00 | 11.39 | D |
| ATOM | 4709 | O | CYS | D | 82 | 4.676 | 70.170 | 55.782 | 1.00 | 10.82 | D |
| ATOM | 4710 | N | CYS | D | 83 | 3.100 | 71.640 | 56.427 | 1.00 | 10.95 | D |
| ATOM | 4711 | CA | CYS | D | 83 | 3.859 | 72.129 | 57.570 | 1.00 | 13.60 | D |
| ATOM | 4712 | CB | CYS | D | 83 | 4.144 | 73.623 | 57.390 | 1.00 | 14.70 | D |
| ATOM | 4713 | SG | CYS | D | 83 | 5.057 | 74.412 | 58.744 | 1.00 | 19.32 | D |
| ATOM | 4714 | C | CYS | D | 83 | 3.086 | 71.902 | 58.868 | 1.00 | 14.07 | D |
| ATOM | 4715 | O | CYS | D | 83 | 1.932 | 72.314 | 58.989 | 1.00 | 12.91 | D |
| ATOM | 4716 | N | LEU | D | 84 | 3.714 | 71.233 | 59.830 | 1.00 | 14.37 | D |
| ATOM | 4717 | CA | LEU | D | 84 | 3.070 | 70.991 | 61.115 | 1.00 | 15.98 | D |
| ATOM | 4718 | CB | LEU | D | 84 | 3.362 | 69.576 | 61.627 | 1.00 | 17.55 | D |
| ATOM | 4719 | CG | LEU | D | 84 | 2.925 | 68.372 | 60.790 | 1.00 | 20.60 | D |
| ATOM | 4720 | CD1 | LEU | D | 84 | 3.124 | 67.103 | 61.599 | 1.00 | 21.79 | D |
| ATOM | 4721 | CD2 | LEU | D | 84 | 1.467 | 68.504 | 60.396 | 1.00 | 19.79 | D |
| ATOM | 4722 | C | LEU | D | 84 | 3.605 | 72.006 | 62.118 | 1.00 | 16.49 | D |
| ATOM | 4723 | O | LEU | D | 84 | 4.683 | 71.817 | 62.683 | 1.00 | 17.99 | D |
| ATOM | 4724 | N | LEU | D | 85 | 2.864 | 73.091 | 62.319 | 1.00 | 14.37 | D |
| ATOM | 4725 | CA | LEU | D | 85 | 3.268 | 74.124 | 63.266 | 1.00 | 11.01 | D |
| ATOM | 4726 | CB | LEU | D | 85 | 2.518 | 75.429 | 62.985 | 1.00 | 14.11 | D |
| ATOM | 4727 | CG | LEU | D | 85 | 2.780 | 76.120 | 61.640 | 1.00 | 14.99 | D |
| ATOM | 4728 | CD1 | LEU | D | 85 | 1.816 | 77.273 | 61.460 | 1.00 | 15.38 | D |
| ATOM | 4729 | CD2 | LEU | D | 85 | 4.217 | 76.618 | 61.576 | 1.00 | 13.61 | D |
| ATOM | 4730 | C | LEU | D | 85 | 2.918 | 73.591 | 64.649 | 1.00 | 10.23 | D |
| ATOM | 4731 | O | LEU | D | 85 | 1.749 | 73.364 | 64.957 | 1.00 | 8.60 | D |
| ATOM | 4732 | N | THR | D | 86 | 3.935 | 73.386 | 65.476 | 1.00 | 9.81 | D |
| ATOM | 4733 | CA | THR | D | 86 | 3.732 | 72.831 | 66.807 | 1.00 | 9.41 | D |
| ATOM | 4734 | CB | THR | D | 86 | 4.610 | 71.568 | 67.014 | 1.00 | 12.20 | D |
| ATOM | 4735 | OG1 | THR | D | 86 | 4.300 | 70.591 | 66.014 | 1.00 | 14.92 | D |
| ATOM | 4736 | CG2 | THR | D | 86 | 4.377 | 70.974 | 68.389 | 1.00 | 12.94 | D |
| ATOM | 4737 | C | THR | D | 86 | 4.060 | 73.786 | 67.945 | 1.00 | 9.29 | D |
| ATOM | 4738 | O | THR | D | 86 | 5.024 | 74.543 | 67.870 | 1.00 | 6.55 | D |
| ATOM | 4739 | N | PHE | D | 87 | 3.239 | 73.750 | 68.990 | 1.00 | 9.26 | D |
| ATOM | 4740 | CA | PHE | D | 87 | 3.478 | 74.550 | 70.186 | 1.00 | 10.61 | D |
| ATOM | 4741 | CB | PHE | D | 87 | 2.606 | 75.820 | 70.226 | 1.00 | 9.65 | D |
| ATOM | 4742 | CG | PHE | D | 87 | 1.138 | 75.564 | 70.445 | 1.00 | 10.51 | D |
| ATOM | 4743 | CD1 | PHE | D | 87 | 0.305 | 75.237 | 69.381 | 1.00 | 11.34 | D |
| ATOM | 4744 | CD2 | PHE | D | 87 | 0.580 | 75.697 | 71.718 | 1.00 | 10.51 | D |
| ATOM | 4745 | CE1 | PHE | D | 87 | −1.066 | 75.051 | 69.576 | 1.00 | 8.25 | D |
| ATOM | 4746 | CE2 | PHE | D | 87 | −0.782 | 75.515 | 71.927 | 1.00 | 10.08 | D |
| ATOM | 4747 | CZ | PHE | D | 87 | −1.613 | 75.191 | 70.851 | 1.00 | 9.63 | D |
| ATOM | 4748 | C | PHE | D | 87 | 3.161 | 73.650 | 71.372 | 1.00 | 9.79 | D |
| ATOM | 4749 | O | PHE | D | 87 | 2.618 | 72.559 | 71.192 | 1.00 | 11.36 | D |
| ATOM | 4750 | N | SER | D | 88 | 3.524 | 74.091 | 72.573 | 1.00 | 8.94 | D |
| ATOM | 4751 | CA | SER | D | 88 | 3.265 | 73.334 | 73.795 | 1.00 | 10.32 | D |
| ATOM | 4752 | CB | SER | D | 88 | 4.537 | 73.255 | 74.649 | 1.00 | 11.02 | D |
| ATOM | 4753 | OG | SER | D | 88 | 4.258 | 72.688 | 75.924 | 1.00 | 11.53 | D |
| ATOM | 4754 | C | SER | D | 88 | 2.157 | 74.028 | 74.587 | 1.00 | 10.55 | D |
| ATOM | 4755 | O | SER | D | 88 | 2.212 | 75.238 | 74.800 | 1.00 | 8.30 | D |
| ATOM | 4756 | N | VAL | D | 89 | 1.154 | 73.274 | 75.029 | 1.00 | 10.69 | D |
| ATOM | 4757 | CA | VAL | D | 89 | 0.064 | 73.885 | 75.784 | 1.00 | 12.73 | D |
| ATOM | 4758 | CB | VAL | D | 89 | −1.106 | 72.904 | 75.998 | 1.00 | 14.00 | D |
| ATOM | 4759 | CG1 | VAL | D | 89 | −1.711 | 72.533 | 74.650 | 1.00 | 14.88 | D |
| ATOM | 4760 | CG2 | VAL | D | 89 | −0.632 | 71.663 | 76.740 | 1.00 | 15.51 | D |
| ATOM | 4761 | C | VAL | D | 89 | 0.532 | 74.428 | 77.130 | 1.00 | 13.72 | D |
| ATOM | 4762 | O | VAL | D | 89 | −0.208 | 75.133 | 77.815 | 1.00 | 13.52 | D |
| ATOM | 4763 | N | ASP | D | 90 | 1.771 | 74.114 | 77.499 | 1.00 | 15.19 | D |
| ATOM | 4764 | CA | ASP | D | 90 | 2.330 | 74.598 | 78.755 | 1.00 | 18.02 | D |
| ATOM | 4765 | CB | ASP | D | 90 | 2.970 | 73.441 | 79.522 | 1.00 | 21.55 | D |
| ATOM | 4766 | CG | ASP | D | 90 | 2.020 | 72.279 | 79.705 | 1.00 | 26.34 | D |
| ATOM | 4767 | OD1 | ASP | D | 90 | 0.857 | 72.520 | 80.098 | 1.00 | 29.34 | D |
| ATOM | 4768 | OD2 | ASP | D | 90 | 2.433 | 71.126 | 79.458 | 1.00 | 31.40 | D |
| ATOM | 4769 | C | ASP | D | 90 | 3.363 | 75.699 | 78.507 | 1.00 | 17.28 | D |
| ATOM | 4770 | O | ASP | D | 90 | 4.127 | 76.064 | 79.402 | 1.00 | 17.32 | D |
| ATOM | 4771 | N | ASP | D | 91 | 3.372 | 76.228 | 77.286 | 1.00 | 16.96 | D |
| ATOM | 4772 | CA | ASP | D | 91 | 4.297 | 77.287 | 76.893 | 1.00 | 15.43 | D |
| ATOM | 4773 | CB | ASP | D | 91 | 5.446 | 76.704 | 76.062 | 1.00 | 18.08 | D |
| ATOM | 4774 | CG | ASP | D | 91 | 6.533 | 77.726 | 75.749 | 1.00 | 20.51 | D |
| ATOM | 4775 | OD1 | ASP | D | 91 | 6.241 | 78.940 | 75.678 | 1.00 | 22.57 | D |
| ATOM | 4776 | OD2 | ASP | D | 91 | 7.691 | 77.306 | 75.551 | 1.00 | 23.78 | D |
| ATOM | 4777 | C | ASP | D | 91 | 3.523 | 78.297 | 76.051 | 1.00 | 14.87 | D |
| ATOM | 4778 | O | ASP | D | 91 | 3.468 | 78.181 | 74.824 | 1.00 | 12.14 | D |
| ATOM | 4779 | N | SER | D | 92 | 2.921 | 79.284 | 76.705 | 1.00 | 12.74 | D |
| ATOM | 4780 | CA | SER | D | 92 | 2.151 | 80.282 | 75.977 | 1.00 | 12.79 | D |
| ATOM | 4781 | CB | SER | D | 92 | 1.510 | 81.283 | 76.949 | 1.00 | 13.47 | D |
| ATOM | 4782 | OG | SER | D | 92 | 2.490 | 81.986 | 77.683 | 1.00 | 17.31 | D |
| ATOM | 4783 | C | SER | D | 92 | 3.011 | 81.017 | 74.948 | 1.00 | 11.86 | D |
| ATOM | 4784 | O | SER | D | 92 | 2.492 | 81.497 | 73.942 | 1.00 | 11.21 | D |
| ATOM | 4785 | N | GLN | D | 93 | 4.317 | 81.097 | 75.188 | 1.00 | 11.23 | D |
| ATOM | 4786 | CA | GLN | D | 93 | 5.204 | 81.779 | 74.248 | 1.00 | 12.74 | D |
| ATOM | 4787 | CB | GLN | D | 93 | 6.616 | 81.908 | 74.826 | 1.00 | 15.98 | D |
| ATOM | 4788 | CG | GLN | D | 93 | 7.542 | 82.773 | 73.976 | 1.00 | 20.49 | D |
| ATOM | 4789 | CD | GLN | D | 93 | 8.858 | 83.093 | 74.668 | 1.00 | 24.29 | D |
| ATOM | 4790 | OE1 | GLN | D | 93 | 9.698 | 82.216 | 74.878 | 1.00 | 26.13 | D |
| ATOM | 4791 | NE2 | GLN | D | 93 | 9.039 | 84.357 | 75.033 | 1.00 | 26.58 | D |
| ATOM | 4792 | C | GLN | D | 93 | 5.260 | 81.024 | 72.920 | 1.00 | 11.75 | D |
| ATOM | 4793 | O | GLN | D | 93 | 5.176 | 81.630 | 71.848 | 1.00 | 10.83 | D |
| ATOM | 4794 | N | SER | D | 94 | 5.402 | 79.702 | 72.996 | 1.00 | 11.56 | D |
| ATOM | 4795 | CA | SER | D | 94 | 5.456 | 78.880 | 71.792 | 1.00 | 9.78 | D |
| ATOM | 4796 | CB | SER | D | 94 | 5.657 | 77.398 | 72.157 | 1.00 | 9.20 | D |
| ATOM | 4797 | OG | SER | D | 94 | 4.537 | 76.865 | 72.842 | 1.00 | 9.49 | D |
| ATOM | 4798 | C | SER | D | 94 | 4.169 | 79.057 | 70.981 | 1.00 | 9.30 | D |
| ATOM | 4799 | O | SER | D | 94 | 4.188 | 78.997 | 69.753 | 1.00 | 9.16 | D |
| ATOM | 4800 | N | PHE | D | 95 | 3.057 | 79.283 | 71.679 | 1.00 | 9.14 | D |
| ATOM | 4801 | CA | PHE | D | 95 | 1.755 | 79.479 | 71.041 | 1.00 | 9.20 | D |
| ATOM | 4802 | CB | PHE | D | 95 | 0.635 | 79.362 | 72.083 | 1.00 | 10.66 | D |
| ATOM | 4803 | CG | PHE | D | 95 | −0.736 | 79.689 | 71.547 | 1.00 | 10.68 | D |
| ATOM | 4804 | CD1 | PHE | D | 95 | −1.343 | 78.873 | 70.598 | 1.00 | 10.76 | D |
| ATOM | 4805 | CD2 | PHE | D | 95 | −1.417 | 80.819 | 71.992 | 1.00 | 12.21 | D |
| ATOM | 4806 | CE1 | PHE | D | 95 | −2.612 | 79.178 | 70.098 | 1.00 | 12.17 | D |
| ATOM | 4807 | CE2 | PHE | D | 95 | −2.681 | 81.136 | 71.502 | 1.00 | 11.29 | D |
| ATOM | 4808 | CZ | PHE | D | 95 | −3.282 | 80.317 | 70.554 | 1.00 | 13.84 | D |
| ATOM | 4809 | C | PHE | D | 95 | 1.680 | 80.846 | 70.364 | 1.00 | 8.88 | D |
| ATOM | 4810 | O | PHE | D | 95 | 1.190 | 80.967 | 69.247 | 1.00 | 7.93 | D |
| ATOM | 4811 | N | GLN | D | 96 | 2.160 | 81.878 | 71.048 | 1.00 | 9.04 | D |
| ATOM | 4812 | CA | GLN | D | 96 | 2.138 | 83.224 | 70.491 | 1.00 | 10.40 | D |
| ATOM | 4813 | CB | GLN | D | 96 | 2.525 | 84.244 | 71.565 | 1.00 | 13.49 | D |
| ATOM | 4814 | CG | GLN | D | 96 | 1.545 | 84.287 | 72.727 | 1.00 | 19.07 | D |
| ATOM | 4815 | CD | GLN | D | 96 | 0.143 | 84.680 | 72.292 | 1.00 | 25.02 | D |
| ATOM | 4816 | OE1 | GLN | D | 96 | −0.815 | 84.569 | 73.062 | 1.00 | 30.41 | D |
| ATOM | 4817 | NE2 | GLN | D | 96 | 0.016 | 85.154 | 71.054 | 1.00 | 29.01 | D |
| ATOM | 4818 | C | GLN | D | 96 | 3.073 | 83.345 | 69.289 | 1.00 | 10.52 | D |
| ATOM | 4819 | O | GLN | D | 96 | 2.891 | 84.211 | 68.436 | 1.00 | 11.74 | D |
| ATOM | 4820 | N | ASN | D | 97 | 4.067 | 82.468 | 69.226 | 1.00 | 8.38 | D |
| ATOM | 4821 | CA | ASN | D | 97 | 5.019 | 82.473 | 68.126 | 1.00 | 8.50 | D |
| ATOM | 4822 | CB | ASN | D | 97 | 6.333 | 81.825 | 68.569 | 1.00 | 9.40 | D |
| ATOM | 4823 | CG | ASN | D | 97 | 7.200 | 82.760 | 69.379 | 1.00 | 13.78 | D |
| ATOM | 4824 | OD1 | ASN | D | 97 | 8.047 | 82.320 | 70.159 | 1.00 | 15.16 | D |
| ATOM | 4825 | ND2 | ASN | D | 97 | 7.006 | 84.061 | 69.188 | 1.00 | 15.01 | D |
| ATOM | 4826 | C | ASN | D | 97 | 4.517 | 81.770 | 66.870 | 1.00 | 8.35 | D |
| ATOM | 4827 | O | ASN | D | 97 | 5.211 | 81.764 | 65.860 | 1.00 | 9.09 | D |
| ATOM | 4828 | N | LEU | D | 98 | 3.324 | 81.179 | 66.917 | 1.00 | 8.30 | D |
| ATOM | 4829 | CA | LEU | D | 98 | 2.801 | 80.482 | 65.741 | 1.00 | 9.58 | D |
| ATOM | 4830 | CB | LEU | D | 98 | 1.391 | 79.946 | 66.007 | 1.00 | 9.30 | D |
| ATOM | 4831 | CG | LEU | D | 98 | 1.255 | 78.773 | 66.980 | 1.00 | 12.16 | D |
| ATOM | 4832 | CD1 | LEU | D | 98 | −0.214 | 78.406 | 67.104 | 1.00 | 12.06 | D |
| ATOM | 4833 | CD2 | LEU | D | 98 | 2.065 | 77.577 | 66.483 | 1.00 | 11.11 | D |
| ATOM | 4834 | C | LEU | D | 98 | 2.779 | 81.381 | 64.503 | 1.00 | 10.90 | D |
| ATOM | 4835 | O | LEU | D | 98 | 3.220 | 80.976 | 63.422 | 1.00 | 10.73 | D |
| ATOM | 4836 | N | SER | D | 99 | 2.267 | 82.599 | 64.658 | 1.00 | 11.06 | D |
| ATOM | 4837 | CA | SER | D | 99 | 2.214 | 83.526 | 63.535 | 1.00 | 12.60 | D |
| ATOM | 4838 | CB | SER | D | 99 | 1.601 | 84.862 | 63.964 | 1.00 | 14.68 | D |
| ATOM | 4839 | OG | SER | D | 99 | 0.212 | 84.718 | 64.214 | 1.00 | 21.02 | D |
| ATOM | 4840 | C | SER | D | 99 | 3.604 | 83.757 | 62.952 | 1.00 | 11.91 | D |
| ATOM | 4841 | O | SER | D | 99 | 3.769 | 83.758 | 61.731 | 1.00 | 12.65 | D |
| ATOM | 4842 | N | ASN | D | 100 | 4.599 | 83.945 | 63.818 | 1.00 | 12.20 | D |
| ATOM | 4843 | CA | ASN | D | 100 | 5.971 | 84.173 | 63.358 | 1.00 | 11.85 | D |
| ATOM | 4844 | CB | ASN | D | 100 | 6.904 | 84.502 | 64.530 | 1.00 | 15.26 | D |
| ATOM | 4845 | CG | ASN | D | 100 | 6.608 | 85.852 | 65.156 | 1.00 | 18.16 | D |
| ATOM | 4846 | OD1 | ASN | D | 100 | 6.337 | 86.828 | 64.455 | 1.00 | 18.70 | D |
| ATOM | 4847 | ND2 | ASN | D | 100 | 6.668 | 85.916 | 66.482 | 1.00 | 17.46 | D |
| ATOM | 4848 | C | ASN | D | 100 | 6.522 | 82.969 | 62.601 | 1.00 | 10.62 | D |
| ATOM | 4849 | O | ASN | D | 100 | 7.259 | 83.131 | 61.627 | 1.00 | 9.13 | D |
| ATOM | 4850 | N | TRP | D | 101 | 6.176 | 81.763 | 63.050 | 1.00 | 9.99 | D |
| ATOM | 4851 | CA | TRP | D | 101 | 6.643 | 80.556 | 62.377 | 1.00 | 8.63 | D |
| ATOM | 4852 | CB | TRP | D | 101 | 6.314 | 79.305 | 63.195 | 1.00 | 8.70 | D |
| ATOM | 4853 | CG | TRP | D | 101 | 7.281 | 79.046 | 64.301 | 1.00 | 6.87 | D |
| ATOM | 4854 | CD2 | TRP | D | 101 | 8.616 | 78.545 | 64.164 | 1.00 | 9.53 | D |
| ATOM | 4855 | CE2 | TRP | D | 101 | 9.173 | 78.482 | 65.458 | 1.00 | 9.74 | D |
| ATOM | 4856 | CE3 | TRP | D | 101 | 9.396 | 78.142 | 63.069 | 1.00 | 12.15 | D |
| ATOM | 4857 | CD1 | TRP | D | 101 | 7.086 | 79.260 | 65.635 | 1.00 | 6.79 | D |
| ATOM | 4858 | NE1 | TRP | D | 101 | 8.218 | 78.924 | 66.337 | 1.00 | 9.57 | D |
| ATOM | 4859 | CZ2 | TRP | D | 101 | 10.480 | 78.031 | 65.692 | 1.00 | 10.92 | D |
| ATOM | 4860 | CZ3 | TRP | D | 101 | 10.697 | 77.692 | 63.303 | 1.00 | 12.37 | D |
| ATOM | 4861 | CH2 | TRP | D | 101 | 11.223 | 77.642 | 64.606 | 1.00 | 12.85 | D |
| ATOM | 4862 | C | TRP | D | 101 | 6.012 | 80.439 | 60.995 | 1.00 | 9.06 | D |
| ATOM | 4863 | O | TRP | D | 101 | 6.680 | 80.064 | 60.033 | 1.00 | 10.14 | D |
| ATOM | 4864 | N | LYS | D | 102 | 4.725 | 80.758 | 60.900 | 1.00 | 9.28 | D |
| ATOM | 4865 | CA | LYS | D | 102 | 4.029 | 80.689 | 59.624 | 1.00 | 10.50 | D |
| ATOM | 4866 | CB | LYS | D | 102 | 2.553 | 81.066 | 59.794 | 1.00 | 10.11 | D |
| ATOM | 4867 | CG | LYS | D | 102 | 1.752 | 81.010 | 58.501 | 1.00 | 13.96 | D |
| ATOM | 4868 | CD | LYS | D | 102 | 0.307 | 81.445 | 58.717 | 1.00 | 15.46 | D |
| ATOM | 4869 | CE | LYS | D | 102 | −0.477 | 81.398 | 57.411 | 1.00 | 17.36 | D |
| ATOM | 4870 | NZ | LYS | D | 102 | −1.899 | 81.825 | 57.584 | 1.00 | 15.80 | D |
| ATOM | 4871 | C | LYS | D | 102 | 4.708 | 81.646 | 58.645 | 1.00 | 9.72 | D |
| ATOM | 4872 | O | LYS | D | 102 | 5.042 | 81.270 | 57.524 | 1.00 | 9.45 | D |
| ATOM | 4873 | N | LYS | D | 103 | 4.935 | 82.877 | 59.087 | 1.00 | 11.02 | D |
| ATOM | 4874 | CA | LYS | D | 103 | 5.579 | 83.876 | 58.243 | 1.00 | 11.08 | D |
| ATOM | 4875 | CB | LYS | D | 103 | 5.697 | 85.205 | 58.991 | 1.00 | 13.25 | D |
| ATOM | 4876 | CG | LYS | D | 103 | 4.362 | 85.856 | 59.292 | 1.00 | 16.21 | D |
| ATOM | 4877 | CD | LYS | D | 103 | 4.554 | 87.187 | 59.998 | 1.00 | 18.95 | D |
| ATOM | 4878 | CE | LYS | D | 103 | 3.219 | 87.854 | 60.275 | 1.00 | 20.66 | D |
| ATOM | 4879 | NZ | LYS | D | 103 | 3.401 | 89.138 | 61.010 | 1.00 | 22.18 | D |
| ATOM | 4880 | C | LYS | D | 103 | 6.963 | 83.429 | 57.789 | 1.00 | 10.96 | D |
| ATOM | 4881 | O | LYS | D | 103 | 7.342 | 83.630 | 56.630 | 1.00 | 11.96 | D |
| ATOM | 4882 | N | GLU | D | 104 | 7.722 | 82.826 | 58.699 | 1.00 | 9.31 | D |
| ATOM | 4883 | CA | GLU | D | 104 | 9.062 | 82.375 | 58.350 | 1.00 | 10.01 | D |
| ATOM | 4884 | CB | GLU | D | 104 | 9.806 | 81.851 | 59.583 | 1.00 | 10.27 | D |
| ATOM | 4885 | CG | GLU | D | 104 | 11.247 | 81.456 | 59.284 | 1.00 | 10.04 | D |
| ATOM | 4886 | CD | GLU | D | 104 | 12.008 | 81.021 | 60.518 | 1.00 | 12.88 | D |
| ATOM | 4887 | OE1 | GLU | D | 104 | 12.165 | 81.845 | 61.445 | 1.00 | 12.66 | D |
| ATOM | 4888 | OE2 | GLU | D | 104 | 12.447 | 79.856 | 60.558 | 1.00 | 11.66 | D |
| ATOM | 4889 | C | GLU | D | 104 | 9.004 | 81.292 | 57.279 | 1.00 | 10.26 | D |
| ATOM | 4890 | O | GLU | D | 104 | 9.749 | 81.337 | 56.302 | 1.00 | 11.06 | D |
| ATOM | 4891 | N | PHE | D | 105 | 8.120 | 80.319 | 57.460 | 1.00 | 10.68 | D |
| ATOM | 4892 | CA | PHE | D | 105 | 7.991 | 79.246 | 56.483 | 1.00 | 11.27 | D |
| ATOM | 4893 | CB | PHE | D | 105 | 6.886 | 78.266 | 56.885 | 1.00 | 11.62 | D |
| ATOM | 4894 | CG | PHE | D | 105 | 6.671 | 77.172 | 55.878 | 1.00 | 10.23 | D |
| ATOM | 4895 | CD1 | PHE | D | 105 | 7.584 | 76.129 | 55.765 | 1.00 | 12.63 | D |
| ATOM | 4896 | CD2 | PHE | D | 105 | 5.596 | 77.220 | 54.996 | 1.00 | 11.53 | D |
| ATOM | 4897 | CE1 | PHE | D | 105 | 7.433 | 75.149 | 54.786 | 1.00 | 12.37 | D |
| ATOM | 4898 | CE2 | PHE | D | 105 | 5.436 | 76.248 | 54.014 | 1.00 | 11.36 | D |
| ATOM | 4899 | CZ | PHE | D | 105 | 6.358 | 75.210 | 53.908 | 1.00 | 11.85 | D |
| ATOM | 4900 | C | PHE | D | 105 | 7.671 | 79.801 | 55.094 | 1.00 | 11.15 | D |
| ATOM | 4901 | O | PHE | D | 105 | 8.372 | 79.518 | 54.129 | 1.00 | 11.03 | D |
| ATOM | 4902 | N | ILE | D | 106 | 6.607 | 80.593 | 55.010 | 1.00 | 12.39 | D |
| ATOM | 4903 | CA | ILE | D | 106 | 6.164 | 81.185 | 53.749 | 1.00 | 13.14 | D |
| ATOM | 4904 | CB | ILE | D | 106 | 4.938 | 82.085 | 53.974 | 1.00 | 16.40 | D |
| ATOM | 4905 | CG2 | ILE | D | 106 | 4.547 | 82.772 | 52.675 | 1.00 | 16.52 | D |
| ATOM | 4906 | CG1 | ILE | D | 106 | 3.776 | 81.250 | 54.512 | 1.00 | 16.02 | D |
| ATOM | 4907 | CD1 | ILE | D | 106 | 2.615 | 82.086 | 55.007 | 1.00 | 20.02 | D |
| ATOM | 4908 | C | ILE | D | 106 | 7.243 | 82.013 | 53.061 | 1.00 | 14.71 | D |
| ATOM | 4909 | O | ILE | D | 106 | 7.399 | 81.965 | 51.836 | 1.00 | 13.74 | D |
| ATOM | 4910 | N | TYR | D | 107 | 7.980 | 82.778 | 53.854 | 1.00 | 13.87 | D |
| ATOM | 4911 | CA | TYR | D | 107 | 9.038 | 83.623 | 53.324 | 1.00 | 15.53 | D |
| ATOM | 4912 | CB | TYR | D | 107 | 9.698 | 84.409 | 54.456 | 1.00 | 16.50 | D |
| ATOM | 4913 | CG | TYR | D | 107 | 10.699 | 85.426 | 53.968 | 1.00 | 20.44 | D |
| ATOM | 4914 | CD1 | TYR | D | 107 | 10.274 | 86.615 | 53.371 | 1.00 | 22.14 | D |
| ATOM | 4915 | CE1 | TYR | D | 107 | 11.190 | 87.553 | 52.901 | 1.00 | 22.66 | D |
| ATOM | 4916 | CD2 | TYR | D | 107 | 12.072 | 85.197 | 54.080 | 1.00 | 20.58 | D |
| ATOM | 4917 | CE2 | TYR | D | 107 | 12.998 | 86.129 | 53.610 | 1.00 | 22.35 | D |
| ATOM | 4918 | CZ | TYR | D | 107 | 12.546 | 87.306 | 53.022 | 1.00 | 23.23 | D |
| ATOM | 4919 | OH | TYR | D | 107 | 13.444 | 88.240 | 52.558 | 1.00 | 26.34 | D |
| ATOM | 4920 | C | TYR | D | 107 | 10.108 | 82.812 | 52.596 | 1.00 | 15.82 | D |
| ATOM | 4921 | O | TYR | D | 107 | 10.505 | 83.149 | 51.482 | 1.00 | 15.26 | D |
| ATOM | 4922 | N | TYR | D | 108 | 10.559 | 81.733 | 53.226 | 1.00 | 14.70 | D |
| ATOM | 4923 | CA | TYR | D | 108 | 11.612 | 80.899 | 52.653 | 1.00 | 14.95 | D |
| ATOM | 4924 | CB | TYR | D | 108 | 12.521 | 80.392 | 53.777 | 1.00 | 14.77 | D |
| ATOM | 4925 | CG | TYR | D | 108 | 13.286 | 81.489 | 54.484 | 1.00 | 17.77 | D |
| ATOM | 4926 | CD1 | TYR | D | 108 | 14.364 | 82.125 | 53.866 | 1.00 | 17.96 | D |
| ATOM | 4927 | CE1 | TYR | D | 108 | 15.067 | 83.143 | 54.512 | 1.00 | 19.29 | D |
| ATOM | 4928 | CD2 | TYR | D | 108 | 12.928 | 81.898 | 55.767 | 1.00 | 17.32 | D |
| ATOM | 4929 | CE2 | TYR | D | 108 | 13.625 | 82.915 | 56.421 | 1.00 | 19.64 | D |
| ATOM | 4930 | CZ | TYR | D | 108 | 14.692 | 83.530 | 55.788 | 1.00 | 20.57 | D |
| ATOM | 4931 | OH | TYR | D | 108 | 15.385 | 84.527 | 56.438 | 1.00 | 22.64 | D |
| ATOM | 4932 | C | TYR | D | 108 | 11.157 | 79.715 | 51.803 | 1.00 | 14.70 | D |
| ATOM | 4933 | O | TYR | D | 108 | 11.939 | 79.189 | 51.017 | 1.00 | 14.45 | D |
| ATOM | 4934 | N | ALA | D | 109 | 9.901 | 79.303 | 51.945 | 1.00 | 16.22 | D |
| ATOM | 4935 | CA | ALA | D | 109 | 9.391 | 78.155 | 51.193 | 1.00 | 18.95 | D |
| ATOM | 4936 | CB | ALA | D | 109 | 8.139 | 77.607 | 51.874 | 1.00 | 18.01 | D |
| ATOM | 4937 | C | ALA | D | 109 | 9.100 | 78.421 | 49.717 | 1.00 | 20.51 | D |
| ATOM | 4938 | O | ALA | D | 109 | 9.050 | 77.485 | 48.917 | 1.00 | 21.75 | D |
| ATOM | 4939 | N | ASP | D | 110 | 8.904 | 79.684 | 49.357 | 1.00 | 23.69 | D |
| ATOM | 4940 | CA | ASP | D | 110 | 8.608 | 80.042 | 47.971 | 1.00 | 26.90 | D |
| ATOM | 4941 | CB | ASP | D | 110 | 9.847 | 79.854 | 47.089 | 1.00 | 30.57 | D |
| ATOM | 4942 | CG | ASP | D | 110 | 10.890 | 80.934 | 47.308 | 1.00 | 34.30 | D |
| ATOM | 4943 | OD1 | ASP | D | 110 | 10.557 | 82.126 | 47.137 | 1.00 | 37.77 | D |
| ATOM | 4944 | OD2 | ASP | D | 110 | 12.043 | 80.594 | 47.648 | 1.00 | 36.62 | D |
| ATOM | 4945 | C | ASP | D | 110 | 7.459 | 79.207 | 47.413 | 1.00 | 26.93 | D |
| ATOM | 4946 | O | ASP | D | 110 | 7.654 | 78.387 | 46.514 | 1.00 | 27.71 | D |
| ATOM | 4947 | N | GLU | D | 115 | −1.901 | 81.699 | 52.078 | 1.00 | 33.46 | D |
| ATOM | 4948 | CA | GLU | D | 115 | −2.662 | 81.406 | 50.870 | 1.00 | 31.64 | D |
| ATOM | 4949 | CB | GLU | D | 115 | −1.720 | 81.306 | 49.671 | 1.00 | 31.86 | D |
| ATOM | 4950 | CG | GLU | D | 115 | −0.440 | 80.547 | 49.943 | 1.00 | 32.52 | D |
| ATOM | 4951 | CD | GLU | D | 115 | 0.317 | 80.241 | 48.669 | 1.00 | 32.99 | D |
| ATOM | 4952 | OE1 | GLU | D | 115 | −0.123 | 79.340 | 47.923 | 1.00 | 31.82 | D |
| ATOM | 4953 | OE2 | GLU | D | 115 | 1.341 | 80.909 | 48.408 | 1.00 | 34.85 | D |
| ATOM | 4954 | C | GLU | D | 115 | −3.478 | 80.124 | 50.999 | 1.00 | 30.57 | D |
| ATOM | 4955 | O | GLU | D | 115 | −4.436 | 80.066 | 51.767 | 1.00 | 30.76 | D |
| ATOM | 4956 | N | SER | D | 116 | −3.104 | 79.098 | 50.243 | 1.00 | 29.09 | D |
| ATOM | 4957 | CA | SER | D | 116 | −3.825 | 77.834 | 50.300 | 1.00 | 28.53 | D |
| ATOM | 4958 | CB | SER | D | 116 | −4.460 | 77.524 | 48.944 | 1.00 | 29.08 | D |
| ATOM | 4959 | OG | SER | D | 116 | −3.470 | 77.321 | 47.950 | 1.00 | 33.14 | D |
| ATOM | 4960 | C | SER | D | 116 | −2.908 | 76.687 | 50.716 | 1.00 | 26.79 | D |
| ATOM | 4961 | O | SER | D | 116 | −3.285 | 75.518 | 50.619 | 1.00 | 27.49 | D |
| ATOM | 4962 | N | PHE | D | 117 | −1.708 | 77.021 | 51.181 | 1.00 | 23.47 | D |
| ATOM | 4963 | CA | PHE | D | 117 | −0.759 | 75.998 | 51.607 | 1.00 | 21.91 | D |
| ATOM | 4964 | CB | PHE | D | 117 | 0.601 | 76.613 | 51.925 | 1.00 | 20.24 | D |
| ATOM | 4965 | CG | PHE | D | 117 | 1.640 | 75.598 | 52.297 | 1.00 | 17.49 | D |
| ATOM | 4966 | CD1 | PHE | D | 117 | 2.306 | 74.873 | 51.314 | 1.00 | 18.05 | D |
| ATOM | 4967 | CD2 | PHE | D | 117 | 1.924 | 75.334 | 53.631 | 1.00 | 17.15 | D |
| ATOM | 4968 | CE1 | PHE | D | 117 | 3.241 | 73.897 | 51.655 | 1.00 | 16.78 | D |
| ATOM | 4969 | CE2 | PHE | D | 117 | 2.856 | 74.361 | 53.981 | 1.00 | 16.66 | D |
| ATOM | 4970 | CZ | PHE | D | 117 | 3.515 | 73.641 | 52.988 | 1.00 | 14.88 | D |
| ATOM | 4971 | C | PHE | D | 117 | −1.274 | 75.278 | 52.849 | 1.00 | 20.60 | D |
| ATOM | 4972 | O | PHE | D | 117 | −1.753 | 75.911 | 53.788 | 1.00 | 20.08 | D |
| ATOM | 4973 | N | PRO | D | 118 | −1.164 | 73.942 | 52.875 | 1.00 | 19.71 | D |
| ATOM | 4974 | CD | PRO | D | 118 | −0.706 | 73.061 | 51.786 | 1.00 | 20.19 | D |
| ATOM | 4975 | CA | PRO | D | 118 | −1.628 | 73.151 | 54.016 | 1.00 | 19.30 | D |
| ATOM | 4976 | CB | PRO | D | 118 | −1.660 | 71.732 | 53.457 | 1.00 | 20.49 | D |
| ATOM | 4977 | CG | PRO | D | 118 | −0.534 | 71.737 | 52.498 | 1.00 | 21.69 | D |
| ATOM | 4978 | C | PRO | D | 118 | −0.784 | 73.252 | 55.280 | 1.00 | 18.17 | D |
| ATOM | 4979 | O | PRO | D | 118 | 0.406 | 72.939 | 55.286 | 1.00 | 18.16 | D |
| ATOM | 4980 | N | PHE | D | 119 | −1.426 | 73.703 | 56.349 | 1.00 | 16.58 | D |
| ATOM | 4981 | CA | PHE | D | 119 | −0.800 | 73.831 | 57.655 | 1.00 | 16.00 | D |
| ATOM | 4982 | CB | PHE | D | 119 | −0.803 | 75.291 | 58.115 | 1.00 | 16.43 | D |
| ATOM | 4983 | CG | PHE | D | 119 | 0.273 | 76.132 | 57.499 | 1.00 | 17.56 | D |
| ATOM | 4984 | CD1 | PHE | D | 119 | 1.582 | 76.053 | 57.959 | 1.00 | 17.59 | D |
| ATOM | 4985 | CD2 | PHE | D | 119 | −0.029 | 77.026 | 56.477 | 1.00 | 18.41 | D |
| ATOM | 4986 | CE1 | PHE | D | 119 | 2.580 | 76.860 | 57.411 | 1.00 | 17.89 | D |
| ATOM | 4987 | CE2 | PHE | D | 119 | 0.960 | 77.837 | 55.922 | 1.00 | 18.53 | D |
| ATOM | 4988 | CZ | PHE | D | 119 | 2.267 | 77.753 | 56.392 | 1.00 | 17.95 | D |
| ATOM | 4989 | C | PHE | D | 119 | −1.655 | 73.022 | 58.622 | 1.00 | 13.94 | D |
| ATOM | 4990 | O | PHE | D | 119 | −2.881 | 73.014 | 58.509 | 1.00 | 14.47 | D |
| ATOM | 4991 | N | VAL | D | 120 | −1.005 | 72.331 | 59.551 | 1.00 | 12.88 | D |
| ATOM | 4992 | CA | VAL | D | 120 | −1.701 | 71.561 | 60.571 | 1.00 | 11.25 | D |
| ATOM | 4993 | CB | VAL | D | 120 | −1.480 | 70.048 | 60.402 | 1.00 | 12.04 | D |
| ATOM | 4994 | CG1 | VAL | D | 120 | −2.158 | 69.294 | 61.534 | 1.00 | 11.67 | D |
| ATOM | 4995 | CG2 | VAL | D | 120 | −2.045 | 69.596 | 59.068 | 1.00 | 11.80 | D |
| ATOM | 4996 | C | VAL | D | 120 | −1.104 | 72.035 | 61.891 | 1.00 | 10.98 | D |
| ATOM | 4997 | O | VAL | D | 120 | 0.116 | 72.010 | 62.076 | 1.00 | 10.55 | D |
| ATOM | 4998 | N | ILE | D | 121 | −1.962 | 72.481 | 62.799 | 1.00 | 8.61 | D |
| ATOM | 4999 | CA | ILE | D | 121 | −1.508 | 72.999 | 64.083 | 1.00 | 7.25 | D |
| ATOM | 5000 | CB | ILE | D | 121 | −2.365 | 74.204 | 64.527 | 1.00 | 8.06 | D |
| ATOM | 5001 | CG2 | ILE | D | 121 | −1.717 | 74.888 | 65.728 | 1.00 | 6.21 | D |
| ATOM | 5002 | CG1 | ILE | D | 121 | −2.526 | 75.189 | 63.364 | 1.00 | 11.01 | D |
| ATOM | 5003 | CD1 | ILE | D | 121 | −1.214 | 75.661 | 62.769 | 1.00 | 11.26 | D |
| ATOM | 5004 | C | ILE | D | 121 | −1.575 | 71.954 | 65.178 | 1.00 | 7.53 | D |
| ATOM | 5005 | O | ILE | D | 121 | −2.609 | 71.312 | 65.370 | 1.00 | 5.65 | D |
| ATOM | 5006 | N | LEU | D | 122 | −0.478 | 71.802 | 65.911 | 1.00 | 7.77 | D |
| ATOM | 5007 | CA | LEU | D | 122 | −0.426 | 70.824 | 66.989 | 1.00 | 9.50 | D |
| ATOM | 5008 | CB | LEU | D | 122 | 0.658 | 69.784 | 66.707 | 1.00 | 11.91 | D |
| ATOM | 5009 | CG | LEU | D | 122 | 0.669 | 69.173 | 65.303 | 1.00 | 14.97 | D |
| ATOM | 5010 | CD1 | LEU | D | 122 | 1.786 | 68.137 | 65.224 | 1.00 | 19.54 | D |
| ATOM | 5011 | CD2 | LEU | D | 122 | −0.675 | 68.544 | 64.995 | 1.00 | 17.93 | D |
| ATOM | 5012 | C | LEU | D | 122 | −0.153 | 71.466 | 68.344 | 1.00 | 9.08 | D |
| ATOM | 5013 | O | LEU | D | 122 | 0.842 | 72.172 | 68.517 | 1.00 | 9.33 | D |
| ATOM | 5014 | N | GLY | D | 123 | −1.058 | 71.225 | 69.290 | 1.00 | 8.13 | D |
| ATOM | 5015 | CA | GLY | D | 123 | −0.905 | 71.733 | 70.642 | 1.00 | 7.28 | D |
| ATOM | 5016 | C | GLY | D | 123 | −0.480 | 70.522 | 71.448 | 1.00 | 8.43 | D |
| ATOM | 5017 | O | GLY | D | 123 | −1.315 | 69.705 | 71.841 | 1.00 | 8.50 | D |
| ATOM | 5018 | N | ASN | D | 124 | 0.823 | 70.411 | 71.695 | 1.00 | 8.32 | D |
| ATOM | 5019 | CA | ASN | D | 124 | 1.385 | 69.259 | 72.391 | 1.00 | 7.89 | D |
| ATOM | 5020 | CB | ASN | D | 124 | 2.772 | 68.974 | 71.793 | 1.00 | 8.58 | D |
| ATOM | 5021 | CG | ASN | D | 124 | 3.305 | 67.611 | 72.168 | 1.00 | 8.13 | D |
| ATOM | 5022 | OD1 | ASN | D | 124 | 2.593 | 66.613 | 72.086 | 1.00 | 8.00 | D |
| ATOM | 5023 | ND2 | ASN | D | 124 | 4.571 | 67.559 | 72.565 | 1.00 | 7.43 | D |
| ATOM | 5024 | C | ASN | D | 124 | 1.470 | 69.326 | 73.922 | 1.00 | 8.31 | D |
| ATOM | 5025 | O | ASN | D | 124 | 1.356 | 70.398 | 74.524 | 1.00 | 7.75 | D |
| ATOM | 5026 | N | LYS | D | 125 | 1.668 | 68.154 | 74.527 | 1.00 | 7.16 | D |
| ATOM | 5027 | CA | LYS | D | 125 | 1.796 | 67.980 | 75.979 | 1.00 | 7.97 | D |
| ATOM | 5028 | CB | LYS | D | 125 | 2.832 | 68.966 | 76.549 | 1.00 | 7.73 | D |
| ATOM | 5029 | CG | LYS | D | 125 | 4.212 | 68.881 | 75.892 | 1.00 | 8.76 | D |
| ATOM | 5030 | CD | LYS | D | 125 | 5.271 | 69.635 | 76.688 | 1.00 | 9.98 | D |
| ATOM | 5031 | CE | LYS | D | 125 | 6.626 | 69.576 | 76.003 | 1.00 | 10.07 | D |
| ATOM | 5032 | NZ | LYS | D | 125 | 7.691 | 70.252 | 76.810 | 1.00 | 10.20 | D |
| ATOM | 5033 | C | LYS | D | 125 | 0.483 | 68.126 | 76.751 | 1.00 | 9.09 | D |
| ATOM | 5034 | O | LYS | D | 125 | 0.483 | 68.568 | 77.907 | 1.00 | 9.05 | D |
| ATOM | 5035 | N | ILE | D | 126 | −0.631 | 67.733 | 76.139 | 1.00 | 11.21 | D |
| ATOM | 5036 | CA | ILE | D | 126 | −1.922 | 67.873 | 76.812 | 1.00 | 11.69 | D |
| ATOM | 5037 | CB | ILE | D | 126 | −3.109 | 67.632 | 75.854 | 1.00 | 12.78 | D |
| ATOM | 5038 | CG2 | ILE | D | 126 | −3.035 | 68.612 | 74.691 | 1.00 | 15.35 | D |
| ATOM | 5039 | CG1 | ILE | D | 126 | −3.106 | 66.185 | 75.361 | 1.00 | 12.59 | D |
| ATOM | 5040 | CD1 | ILE | D | 126 | −4.372 | 65.793 | 74.628 | 1.00 | 14.79 | D |
| ATOM | 5041 | C | ILE | D | 126 | −2.089 | 66.960 | 78.021 | 1.00 | 12.05 | D |
| ATOM | 5042 | O | ILE | D | 126 | −3.075 | 67.064 | 78.745 | 1.00 | 10.82 | D |
| ATOM | 5043 | N | ASP | D | 127 | −1.129 | 66.068 | 78.239 | 1.00 | 12.29 | D |
| ATOM | 5044 | CA | ASP | D | 127 | −1.189 | 65.168 | 79.383 | 1.00 | 12.87 | D |
| ATOM | 5045 | CB | ASP | D | 127 | −0.127 | 64.074 | 79.257 | 1.00 | 13.54 | D |
| ATOM | 5046 | CG | ASP | D | 127 | 1.242 | 64.628 | 78.916 | 1.00 | 15.38 | D |
| ATOM | 5047 | OD1 | ASP | D | 127 | 1.459 | 64.979 | 77.737 | 1.00 | 13.63 | D |
| ATOM | 5048 | OD2 | ASP | D | 127 | 2.097 | 64.720 | 79.827 | 1.00 | 16.23 | D |
| ATOM | 5049 | C | ASP | D | 127 | −0.970 | 65.951 | 80.678 | 1.00 | 12.13 | D |
| ATOM | 5050 | O | ASP | D | 127 | −1.338 | 65.492 | 81.760 | 1.00 | 12.58 | D |
| ATOM | 5051 | N | ILE | D | 128 | −0.368 | 67.132 | 80.564 | 1.00 | 12.27 | D |
| ATOM | 5052 | CA | ILE | D | 128 | −0.106 | 67.974 | 81.731 | 1.00 | 13.36 | D |
| ATOM | 5053 | CB | ILE | D | 128 | 1.086 | 68.925 | 81.468 | 1.00 | 13.64 | D |
| ATOM | 5054 | CG2 | ILE | D | 128 | 1.255 | 69.895 | 82.635 | 1.00 | 16.40 | D |
| ATOM | 5055 | CG1 | ILE | D | 128 | 2.364 | 68.101 | 81.272 | 1.00 | 15.48 | D |
| ATOM | 5056 | CD1 | ILE | D | 128 | 3.587 | 68.923 | 80.914 | 1.00 | 18.20 | D |
| ATOM | 5057 | C | ILE | D | 128 | −1.344 | 68.786 | 82.103 | 1.00 | 13.72 | D |
| ATOM | 5058 | O | ILE | D | 128 | −1.902 | 69.501 | 81.272 | 1.00 | 15.72 | D |
| ATOM | 5059 | N | SER | D | 129 | −1.765 | 68.674 | 83.361 | 1.00 | 13.56 | D |
| ATOM | 5060 | CA | SER | D | 129 | −2.960 | 69.369 | 83.842 | 1.00 | 13.83 | D |
| ATOM | 5061 | CB | SER | D | 129 | −3.354 | 68.829 | 85.220 | 1.00 | 16.03 | D |
| ATOM | 5062 | OG | SER | D | 129 | −2.317 | 69.037 | 86.161 | 1.00 | 22.58 | D |
| ATOM | 5063 | C | SER | D | 129 | −2.889 | 70.896 | 83.901 | 1.00 | 11.69 | D |
| ATOM | 5064 | O | SER | D | 129 | −3.848 | 71.573 | 83.530 | 1.00 | 12.94 | D |
| ATOM | 5065 | N | GLU | D | 130 | −1.774 | 71.450 | 84.366 | 1.00 | 10.71 | D |
| ATOM | 5066 | CA | GLU | D | 130 | −1.671 | 72.905 | 84.452 | 1.00 | 10.29 | D |
| ATOM | 5067 | CB | GLU | D | 130 | −0.748 | 73.314 | 85.605 | 1.00 | 8.78 | D |
| ATOM | 5068 | CG | GLU | D | 130 | −0.857 | 74.790 | 85.963 | 1.00 | 7.54 | D |
| ATOM | 5069 | CD | GLU | D | 130 | −0.210 | 75.121 | 87.297 | 1.00 | 7.52 | D |
| ATOM | 5070 | OE1 | GLU | D | 130 | −0.332 | 74.304 | 88.239 | 1.00 | 7.34 | D |
| ATOM | 5071 | OE2 | GLU | D | 130 | 0.406 | 76.203 | 87.410 | 1.00 | 7.29 | D |
| ATOM | 5072 | C | GLU | D | 130 | −1.169 | 73.481 | 83.131 | 1.00 | 10.79 | D |
| ATOM | 5073 | O | GLU | D | 130 | 0.020 | 73.412 | 82.822 | 1.00 | 11.41 | D |
| ATOM | 5074 | N | ARG | D | 131 | −2.090 | 74.059 | 82.365 | 1.00 | 12.80 | D |
| ATOM | 5075 | CA | ARG | D | 131 | −1.788 | 74.623 | 81.049 | 1.00 | 13.55 | D |
| ATOM | 5076 | CB | ARG | D | 131 | −2.842 | 74.142 | 80.049 | 1.00 | 13.72 | D |
| ATOM | 5077 | CG | ARG | D | 131 | −2.924 | 72.637 | 79.914 | 1.00 | 14.91 | D |
| ATOM | 5078 | CD | ARG | D | 131 | −4.172 | 72.213 | 79.149 | 1.00 | 16.43 | D |
| ATOM | 5079 | NE | ARG | D | 131 | −4.329 | 72.941 | 77.893 | 1.00 | 16.62 | D |
| ATOM | 5080 | CZ | ARG | D | 131 | −4.884 | 72.425 | 76.801 | 1.00 | 16.60 | D |
| ATOM | 5081 | NH1 | ARG | D | 131 | −5.330 | 71.174 | 76.813 | 1.00 | 16.90 | D |
| ATOM | 5082 | NH2 | ARG | D | 131 | −5.000 | 73.157 | 75.700 | 1.00 | 13.65 | D |
| ATOM | 5083 | C | ARG | D | 131 | −1.716 | 76.146 | 80.983 | 1.00 | 13.92 | D |
| ATOM | 5084 | O | ARG | D | 131 | −2.354 | 76.847 | 81.768 | 1.00 | 12.95 | D |
| ATOM | 5085 | N | GLN | D | 132 | −0.939 | 76.653 | 80.028 | 1.00 | 14.56 | D |
| ATOM | 5086 | CA | GLN | D | 132 | −0.802 | 78.094 | 79.840 | 1.00 | 14.52 | D |
| ATOM | 5087 | CB | GLN | D | 132 | 0.657 | 78.479 | 79.597 | 1.00 | 16.31 | D |
| ATOM | 5088 | CG | GLN | D | 132 | 1.569 | 78.277 | 80.788 | 1.00 | 21.92 | D |
| ATOM | 5089 | CD | GLN | D | 132 | 2.765 | 79.202 | 80.748 | 1.00 | 23.57 | D |
| ATOM | 5090 | OE1 | GLN | D | 132 | 3.505 | 79.245 | 79.760 | 1.00 | 27.00 | D |
| ATOM | 5091 | NE2 | GLN | D | 132 | 2.960 | 79.959 | 81.821 | 1.00 | 23.80 | D |
| ATOM | 5092 | C | GLN | D | 132 | −1.637 | 78.542 | 78.651 | 1.00 | 14.02 | D |
| ATOM | 5093 | O | GLN | D | 132 | −1.864 | 79.737 | 78.457 | 1.00 | 14.03 | D |
| ATOM | 5094 | N | VAL | D | 133 | −2.077 | 77.573 | 77.851 | 1.00 | 13.42 | D |
| ATOM | 5095 | CA | VAL | D | 133 | −2.896 | 77.835 | 76.671 | 1.00 | 14.33 | D |
| ATOM | 5096 | CB | VAL | D | 133 | −2.109 | 77.557 | 75.367 | 1.00 | 12.86 | D |
| ATOM | 5097 | CG1 | VAL | D | 133 | −2.980 | 77.877 | 74.153 | 1.00 | 11.04 | D |
| ATOM | 5098 | CG2 | VAL | D | 133 | −0.836 | 78.381 | 75.348 | 1.00 | 13.66 | D |
| ATOM | 5099 | C | VAL | D | 133 | −4.113 | 76.919 | 76.703 | 1.00 | 14.18 | D |
| ATOM | 5100 | O | VAL | D | 133 | −3.972 | 75.700 | 76.736 | 1.00 | 13.70 | D |
| ATOM | 5101 | N | SER | D | 134 | −5.308 | 77.500 | 76.679 | 1.00 | 14.54 | D |
| ATOM | 5102 | CA | SER | D | 134 | −6.523 | 76.692 | 76.726 | 1.00 | 17.02 | D |
| ATOM | 5103 | CB | SER | D | 134 | −7.680 | 77.492 | 77.326 | 1.00 | 17.46 | D |
| ATOM | 5104 | OG | SER | D | 134 | −8.174 | 78.435 | 76.392 | 1.00 | 20.27 | D |
| ATOM | 5105 | C | SER | D | 134 | −6.942 | 76.180 | 75.357 | 1.00 | 17.16 | D |
| ATOM | 5106 | O | SER | D | 134 | −6.573 | 76.739 | 74.326 | 1.00 | 16.62 | D |
| ATOM | 5107 | N | THR | D | 135 | −7.725 | 75.108 | 75.365 | 1.00 | 19.39 | D |
| ATOM | 5108 | CA | THR | D | 135 | −8.226 | 74.507 | 74.139 | 1.00 | 21.20 | D |
| ATOM | 5109 | CB | THR | D | 135 | −9.127 | 73.305 | 74.458 | 1.00 | 20.97 | D |
| ATOM | 5110 | OG1 | THR | D | 135 | −8.377 | 72.337 | 75.198 | 1.00 | 20.47 | D |
| ATOM | 5111 | CG2 | THR | D | 135 | −9.649 | 72.665 | 73.178 | 1.00 | 22.66 | D |
| ATOM | 5112 | C | THR | D | 135 | −9.029 | 75.541 | 73.355 | 1.00 | 22.15 | D |
| ATOM | 5113 | O | THR | D | 135 | −8.943 | 75.608 | 72.130 | 1.00 | 22.15 | D |
| ATOM | 5114 | N | GLU | D | 136 | −9.802 | 76.350 | 74.074 | 1.00 | 22.78 | D |
| ATOM | 5115 | CA | GLU | D | 136 | −10.625 | 77.388 | 73.461 | 1.00 | 24.52 | D |
| ATOM | 5116 | CB | GLU | D | 136 | −11.479 | 78.083 | 74.528 | 1.00 | 26.99 | D |
| ATOM | 5117 | CG | GLU | D | 136 | −12.548 | 77.200 | 75.153 | 1.00 | 28.45 | D |
| ATOM | 5118 | CD | GLU | D | 136 | −11.990 | 75.911 | 75.734 | 1.00 | 30.98 | D |
| ATOM | 5119 | OE1 | GLU | D | 136 | −11.056 | 75.976 | 76.566 | 1.00 | 29.04 | D |
| ATOM | 5120 | OE2 | GLU | D | 136 | −12.492 | 74.829 | 75.358 | 1.00 | 32.09 | D |
| ATOM | 5121 | C | GLU | D | 136 | −9.788 | 78.431 | 72.721 | 1.00 | 24.43 | D |
| ATOM | 5122 | O | GLU | D | 136 | −10.135 | 78.839 | 71.614 | 1.00 | 24.01 | D |
| ATOM | 5123 | N | GLU | D | 137 | −8.693 | 78.865 | 73.338 | 1.00 | 24.89 | D |
| ATOM | 5124 | CA | GLU | D | 137 | −7.814 | 79.856 | 72.723 | 1.00 | 25.44 | D |
| ATOM | 5125 | CB | GLU | D | 137 | −6.639 | 80.188 | 73.644 | 1.00 | 28.39 | D |
| ATOM | 5126 | CG | GLU | D | 137 | −7.005 | 80.711 | 75.009 | 1.00 | 33.06 | D |
| ATOM | 5127 | CD | GLU | D | 137 | −5.774 | 81.014 | 75.841 | 1.00 | 35.28 | D |
| ATOM | 5128 | OE1 | GLU | D | 137 | −4.980 | 81.885 | 75.422 | 1.00 | 36.68 | D |
| ATOM | 5129 | OE2 | GLU | D | 137 | −5.598 | 80.381 | 76.905 | 1.00 | 35.77 | D |
| ATOM | 5130 | C | GLU | D | 137 | −7.246 | 79.311 | 71.421 | 1.00 | 24.05 | D |
| ATOM | 5131 | O | GLU | D | 137 | −7.340 | 79.943 | 70.368 | 1.00 | 22.74 | D |
| ATOM | 5132 | N | ALA | D | 138 | −6.640 | 78.133 | 71.515 | 1.00 | 22.91 | D |
| ATOM | 5133 | CA | ALA | D | 138 | −6.031 | 77.477 | 70.367 | 1.00 | 21.97 | D |
| ATOM | 5134 | CB | ALA | D | 138 | −5.486 | 76.116 | 70.780 | 1.00 | 20.01 | D |
| ATOM | 5135 | C | ALA | D | 138 | −7.002 | 77.318 | 69.205 | 1.00 | 21.34 | D |
| ATOM | 5136 | O | ALA | D | 138 | −6.683 | 77.669 | 68.071 | 1.00 | 21.99 | D |
| ATOM | 5137 | N | GLN | D | 139 | −8.181 | 76.777 | 69.487 | 1.00 | 21.99 | D |
| ATOM | 5138 | CA | GLN | D | 139 | −9.187 | 76.580 | 68.451 | 1.00 | 21.97 | D |
| ATOM | 5139 | CB | GLN | D | 139 | −10.429 | 75.912 | 69.043 | 1.00 | 23.28 | D |
| ATOM | 5140 | CG | GLN | D | 139 | −10.171 | 74.531 | 69.615 | 1.00 | 26.22 | D |
| ATOM | 5141 | CD | GLN | D | 139 | −11.397 | 73.941 | 70.280 | 1.00 | 27.96 | D |
| ATOM | 5142 | OE1 | GLN | D | 139 | −11.986 | 74.550 | 71.172 | 1.00 | 31.40 | D |
| ATOM | 5143 | NE2 | GLN | D | 139 | −11.786 | 72.748 | 69.853 | 1.00 | 30.22 | D |
| ATOM | 5144 | C | GLN | D | 139 | −9.571 | 77.909 | 67.810 | 1.00 | 21.84 | D |
| ATOM | 5145 | O | GLN | D | 139 | −9.745 | 77.995 | 66.596 | 1.00 | 21.05 | D |
| ATOM | 5146 | N | ALA | D | 140 | −9.700 | 78.945 | 68.630 | 1.00 | 22.04 | D |
| ATOM | 5147 | CA | ALA | D | 140 | −10.064 | 80.263 | 68.126 | 1.00 | 23.07 | D |
| ATOM | 5148 | CB | ALA | D | 140 | −10.227 | 81.239 | 69.285 | 1.00 | 23.60 | D |
| ATOM | 5149 | C | ALA | D | 140 | −9.002 | 80.768 | 67.156 | 1.00 | 23.91 | D |
| ATOM | 5150 | O | ALA | D | 140 | −9.315 | 81.171 | 66.033 | 1.00 | 25.05 | D |
| ATOM | 5151 | N | TRP | D | 141 | −7.744 | 80.737 | 67.588 | 1.00 | 23.63 | D |
| ATOM | 5152 | CA | TRP | D | 141 | −6.645 | 81.194 | 66.747 | 1.00 | 23.86 | D |
| ATOM | 5153 | CB | TRP | D | 141 | −5.298 | 80.955 | 67.436 | 1.00 | 24.31 | D |
| ATOM | 5154 | CG | TRP | D | 141 | −4.146 | 81.537 | 66.668 | 1.00 | 23.63 | D |
| ATOM | 5155 | CD2 | TRP | D | 141 | −3.360 | 80.877 | 65.667 | 1.00 | 23.42 | D |
| ATOM | 5156 | CE2 | TRP | D | 141 | −2.444 | 81.826 | 65.162 | 1.00 | 23.86 | D |
| ATOM | 5157 | CE3 | TRP | D | 141 | −3.343 | 79.576 | 65.145 | 1.00 | 23.24 | D |
| ATOM | 5158 | CD1 | TRP | D | 141 | −3.685 | 82.820 | 66.729 | 1.00 | 23.36 | D |
| ATOM | 5159 | NE1 | TRP | D | 141 | −2.665 | 83.002 | 65.829 | 1.00 | 22.98 | D |
| ATOM | 5160 | CZ2 | TRP | D | 141 | −1.519 | 81.517 | 64.160 | 1.00 | 22.64 | D |
| ATOM | 5161 | CZ3 | TRP | D | 141 | −2.422 | 79.268 | 64.147 | 1.00 | 22.14 | D |
| ATOM | 5162 | CH2 | TRP | D | 141 | −1.522 | 80.236 | 63.666 | 1.00 | 24.34 | D |
| ATOM | 5163 | C | TRP | D | 141 | −6.643 | 80.469 | 65.407 | 1.00 | 24.30 | D |
| ATOM | 5164 | O | TRP | D | 141 | −6.503 | 81.091 | 64.354 | 1.00 | 24.79 | D |
| ATOM | 5165 | N | CYS | D | 142 | −6.793 | 79.149 | 65.453 | 1.00 | 23.98 | D |
| ATOM | 5166 | CA | CYS | D | 142 | −6.795 | 78.345 | 64.240 | 1.00 | 24.13 | D |
| ATOM | 5167 | CB | CYS | D | 142 | −6.859 | 76.856 | 64.591 | 1.00 | 22.52 | D |
| ATOM | 5168 | SG | CYS | D | 142 | −5.365 | 76.216 | 65.402 | 1.00 | 19.33 | D |
| ATOM | 5169 | C | CYS | D | 142 | −7.955 | 78.715 | 63.321 | 1.00 | 25.25 | D |
| ATOM | 5170 | O | CYS | D | 142 | −7.795 | 78.750 | 62.100 | 1.00 | 24.50 | D |
| ATOM | 5171 | N | ARG | D | 143 | −9.116 | 78.992 | 63.908 | 1.00 | 26.63 | D |
| ATOM | 5172 | CA | ARG | D | 143 | −10.293 | 79.360 | 63.125 | 1.00 | 28.39 | D |
| ATOM | 5173 | CB | ARG | D | 143 | −11.555 | 79.369 | 63.996 | 1.00 | 29.10 | D |
| ATOM | 5174 | CG | ARG | D | 143 | −11.986 | 78.021 | 64.542 | 1.00 | 30.66 | D |
| ATOM | 5175 | CD | ARG | D | 143 | −13.442 | 78.079 | 64.995 | 1.00 | 32.14 | D |
| ATOM | 5176 | NE | ARG | D | 143 | −13.673 | 79.084 | 66.030 | 1.00 | 32.40 | D |
| ATOM | 5177 | CZ | ARG | D | 143 | −13.424 | 78.903 | 67.324 | 1.00 | 32.59 | D |
| ATOM | 5178 | NH1 | ARG | D | 143 | −12.935 | 77.748 | 67.753 | 1.00 | 33.31 | D |
| ATOM | 5179 | NH2 | ARG | D | 143 | −13.663 | 79.878 | 68.190 | 1.00 | 32.90 | D |
| ATOM | 5180 | C | ARG | D | 143 | −10.148 | 80.737 | 62.490 | 1.00 | 29.54 | D |
| ATOM | 5181 | O | ARG | D | 143 | −10.711 | 80.998 | 61.428 | 1.00 | 29.53 | D |
| ATOM | 5182 | N | ASP | D | 144 | −9.391 | 81.614 | 63.141 | 1.00 | 30.64 | D |
| ATOM | 5183 | CA | ASP | D | 144 | −9.214 | 82.972 | 62.644 | 1.00 | 31.84 | D |
| ATOM | 5184 | CB | ASP | D | 144 | −9.365 | 83.963 | 63.802 | 1.00 | 33.24 | D |
| ATOM | 5185 | CG | ASP | D | 144 | −10.644 | 83.747 | 64.586 | 1.00 | 34.48 | D |
| ATOM | 5186 | OD1 | ASP | D | 144 | −11.696 | 83.514 | 63.951 | 1.00 | 34.84 | D |
| ATOM | 5187 | OD2 | ASP | D | 144 | −10.602 | 83.817 | 65.835 | 1.00 | 34.91 | D |
| ATOM | 5188 | C | ASP | D | 144 | −7.903 | 83.251 | 61.912 | 1.00 | 32.04 | D |
| ATOM | 5189 | O | ASP | D | 144 | −7.600 | 84.405 | 61.604 | 1.00 | 33.38 | D |
| ATOM | 5190 | N | ASN | D | 145 | −7.130 | 82.209 | 61.621 | 1.00 | 30.87 | D |
| ATOM | 5191 | CA | ASN | D | 145 | −5.863 | 82.398 | 60.923 | 1.00 | 29.58 | D |
| ATOM | 5192 | CB | ASN | D | 145 | −4.706 | 82.316 | 61.917 | 1.00 | 30.49 | D |
| ATOM | 5193 | CG | ASN | D | 145 | −4.582 | 83.566 | 62.759 | 1.00 | 31.87 | D |
| ATOM | 5194 | OD1 | ASN | D | 145 | −4.017 | 84.567 | 62.318 | 1.00 | 34.05 | D |
| ATOM | 5195 | ND2 | ASN | D | 145 | −5.125 | 83.524 | 63.970 | 1.00 | 30.79 | D |
| ATOM | 5196 | C | ASN | D | 145 | −5.639 | 81.414 | 59.785 | 1.00 | 28.43 | D |
| ATOM | 5197 | O | ASN | D | 145 | −4.508 | 81.013 | 59.514 | 1.00 | 30.32 | D |
| ATOM | 5198 | N | GLY | D | 146 | −6.720 | 81.034 | 59.114 | 1.00 | 26.67 | D |
| ATOM | 5199 | CA | GLY | D | 146 | −6.610 | 80.102 | 58.007 | 1.00 | 24.38 | D |
| ATOM | 5200 | C | GLY | D | 146 | −7.485 | 78.880 | 58.201 | 1.00 | 23.67 | D |
| ATOM | 5201 | O | GLY | D | 146 | −7.615 | 78.055 | 57.301 | 1.00 | 23.50 | D |
| ATOM | 5202 | N | ASP | D | 147 | −8.090 | 78.779 | 59.380 | 1.00 | 23.16 | D |
| ATOM | 5203 | CA | ASP | D | 147 | −8.957 | 77.659 | 59.739 | 1.00 | 22.97 | D |
| ATOM | 5204 | CB | ASP | D | 147 | −10.262 | 77.705 | 58.937 | 1.00 | 25.45 | D |
| ATOM | 5205 | CG | ASP | D | 147 | −11.290 | 76.709 | 59.445 | 1.00 | 27.55 | D |
| ATOM | 5206 | OD1 | ASP | D | 147 | −11.494 | 76.642 | 60.678 | 1.00 | 28.85 | D |
| ATOM | 5207 | OD2 | ASP | D | 147 | −11.899 | 75.997 | 58.616 | 1.00 | 29.96 | D |
| ATOM | 5208 | C | ASP | D | 147 | −8.257 | 76.320 | 59.533 | 1.00 | 20.98 | D |
| ATOM | 5209 | O | ASP | D | 147 | −8.824 | 75.376 | 58.980 | 1.00 | 21.60 | D |
| ATOM | 5210 | N | TYR | D | 148 | −7.013 | 76.247 | 59.989 | 1.00 | 16.66 | D |
| ATOM | 5211 | CA | TYR | D | 148 | −6.231 | 75.026 | 59.871 | 1.00 | 15.96 | D |
| ATOM | 5212 | CB | TYR | D | 148 | −4.757 | 75.317 | 60.137 | 1.00 | 15.81 | D |
| ATOM | 5213 | CG | TYR | D | 148 | −4.181 | 76.403 | 59.264 | 1.00 | 16.23 | D |
| ATOM | 5214 | CD1 | TYR | D | 148 | −4.321 | 76.353 | 57.880 | 1.00 | 18.75 | D |
| ATOM | 5215 | CE1 | TYR | D | 148 | −3.767 | 77.336 | 57.064 | 1.00 | 20.02 | D |
| ATOM | 5216 | CD2 | TYR | D | 148 | −3.473 | 77.467 | 59.819 | 1.00 | 19.08 | D |
| ATOM | 5217 | CE2 | TYR | D | 148 | −2.913 | 78.457 | 59.013 | 1.00 | 20.05 | D |
| ATOM | 5218 | CZ | TYR | D | 148 | −3.065 | 78.382 | 57.637 | 1.00 | 19.77 | D |
| ATOM | 5219 | OH | TYR | D | 148 | −2.515 | 79.348 | 56.829 | 1.00 | 23.89 | D |
| ATOM | 5220 | C | TYR | D | 148 | −6.708 | 73.986 | 60.873 | 1.00 | 13.96 | D |
| ATOM | 5221 | O | TYR | D | 148 | −7.232 | 74.323 | 61.934 | 1.00 | 14.58 | D |
| ATOM | 5222 | N | PRO | D | 149 | −6.544 | 72.701 | 60.542 | 1.00 | 13.99 | D |
| ATOM | 5223 | CD | PRO | D | 149 | −6.026 | 72.099 | 59.302 | 1.00 | 13.52 | D |
| ATOM | 5224 | CA | PRO | D | 149 | −6.980 | 71.674 | 61.488 | 1.00 | 12.66 | D |
| ATOM | 5225 | CB | PRO | D | 149 | −6.796 | 70.374 | 60.702 | 1.00 | 13.82 | D |
| ATOM | 5226 | CG | PRO | D | 149 | −5.699 | 70.695 | 59.748 | 1.00 | 14.79 | D |
| ATOM | 5227 | C | PRO | D | 149 | −6.098 | 71.759 | 62.729 | 1.00 | 12.01 | D |
| ATOM | 5228 | O | PRO | D | 149 | −4.892 | 72.010 | 62.637 | 1.00 | 11.23 | D |
| ATOM | 5229 | N | TYR | D | 150 | −6.708 | 71.568 | 63.890 | 1.00 | 10.29 | D |
| ATOM | 5230 | CA | TYR | D | 150 | −5.987 | 71.646 | 65.147 | 1.00 | 9.57 | D |
| ATOM | 5231 | CB | TYR | D | 150 | −6.545 | 72.805 | 65.978 | 1.00 | 10.90 | D |
| ATOM | 5232 | CG | TYR | D | 150 | −6.013 | 72.860 | 67.387 | 1.00 | 11.71 | D |
| ATOM | 5233 | CD1 | TYR | D | 150 | −4.662 | 73.104 | 67.634 | 1.00 | 10.26 | D |
| ATOM | 5234 | CE1 | TYR | D | 150 | −4.168 | 73.149 | 68.931 | 1.00 | 12.31 | D |
| ATOM | 5235 | CD2 | TYR | D | 150 | −6.859 | 72.661 | 68.477 | 1.00 | 13.62 | D |
| ATOM | 5236 | CE2 | TYR | D | 150 | −6.375 | 72.705 | 69.778 | 1.00 | 14.32 | D |
| ATOM | 5237 | CZ | TYR | D | 150 | −5.028 | 72.949 | 69.996 | 1.00 | 14.80 | D |
| ATOM | 5238 | OH | TYR | D | 150 | −4.542 | 73.004 | 71.283 | 1.00 | 14.30 | D |
| ATOM | 5239 | C | TYR | D | 150 | −6.084 | 70.348 | 65.933 | 1.00 | 9.81 | D |
| ATOM | 5240 | O | TYR | D | 150 | −7.162 | 69.765 | 66.055 | 1.00 | 8.77 | D |
| ATOM | 5241 | N | PHE | D | 151 | −4.951 | 69.894 | 66.459 | 1.00 | 8.67 | D |
| ATOM | 5242 | CA | PHE | D | 151 | −4.916 | 68.669 | 67.248 | 1.00 | 9.10 | D |
| ATOM | 5243 | CB | PHE | D | 151 | −4.168 | 67.555 | 66.509 | 1.00 | 7.65 | D |
| ATOM | 5244 | CG | PHE | D | 151 | −4.837 | 67.108 | 65.248 | 1.00 | 8.14 | D |
| ATOM | 5245 | CD1 | PHE | D | 151 | −4.590 | 67.759 | 64.045 | 1.00 | 8.49 | D |
| ATOM | 5246 | CD2 | PHE | D | 151 | −5.738 | 66.053 | 65.269 | 1.00 | 8.93 | D |
| ATOM | 5247 | CE1 | PHE | D | 151 | −5.240 | 67.362 | 62.874 | 1.00 | 9.00 | D |
| ATOM | 5248 | CE2 | PHE | D | 151 | −6.392 | 65.647 | 64.107 | 1.00 | 10.94 | D |
| ATOM | 5249 | CZ | PHE | D | 151 | −6.142 | 66.306 | 62.906 | 1.00 | 11.64 | D |
| ATOM | 5250 | C | PHE | D | 151 | −4.233 | 68.868 | 68.588 | 1.00 | 8.28 | D |
| ATOM | 5251 | O | PHE | D | 151 | −3.159 | 69.460 | 68.661 | 1.00 | 10.37 | D |
| ATOM | 5252 | N | GLU | D | 152 | −4.866 | 68.379 | 69.648 | 1.00 | 8.77 | D |
| ATOM | 5253 | CA | GLU | D | 152 | −4.264 | 68.448 | 70.968 | 1.00 | 9.93 | D |
| ATOM | 5254 | CB | GLU | D | 152 | −5.330 | 68.621 | 72.056 | 1.00 | 10.16 | D |
| ATOM | 5255 | CG | GLU | D | 152 | −6.072 | 69.961 | 71.967 | 1.00 | 15.02 | D |
| ATOM | 5256 | CD | GLU | D | 152 | −6.080 | 70.734 | 73.282 | 1.00 | 14.79 | D |
| ATOM | 5257 | OE1 | GLU | D | 152 | −6.403 | 70.134 | 74.327 | 1.00 | 16.00 | D |
| ATOM | 5258 | OE2 | GLU | D | 152 | −5.772 | 71.947 | 73.269 | 1.00 | 16.84 | D |
| ATOM | 5259 | C | GLU | D | 152 | −3.577 | 67.090 | 71.060 | 1.00 | 8.49 | D |
| ATOM | 5260 | O | GLU | D | 152 | −4.228 | 66.042 | 71.053 | 1.00 | 10.18 | D |
| ATOM | 5261 | N | THR | D | 153 | −2.254 | 67.119 | 71.118 | 1.00 | 8.92 | D |
| ATOM | 5262 | CA | THR | D | 153 | −1.477 | 65.895 | 71.141 | 1.00 | 7.66 | D |
| ATOM | 5263 | CB | THR | D | 153 | −0.512 | 65.858 | 69.944 | 1.00 | 11.09 | D |
| ATOM | 5264 | OG1 | THR | D | 153 | 0.497 | 66.860 | 70.119 | 1.00 | 9.31 | D |
| ATOM | 5265 | CG2 | THR | D | 153 | −1.260 | 66.138 | 68.645 | 1.00 | 6.87 | D |
| ATOM | 5266 | C | THR | D | 153 | −0.646 | 65.696 | 72.389 | 1.00 | 9.77 | D |
| ATOM | 5267 | O | THR | D | 153 | −0.468 | 66.606 | 73.200 | 1.00 | 8.01 | D |
| ATOM | 5268 | N | SER | D | 154 | −0.139 | 64.478 | 72.524 | 1.00 | 9.08 | D |
| ATOM | 5269 | CA | SER | D | 154 | 0.723 | 64.115 | 73.626 | 1.00 | 8.64 | D |
| ATOM | 5270 | CB | SER | D | 154 | −0.077 | 63.610 | 74.827 | 1.00 | 9.78 | D |
| ATOM | 5271 | OG | SER | D | 154 | 0.810 | 63.117 | 75.826 | 1.00 | 9.18 | D |
| ATOM | 5272 | C | SER | D | 154 | 1.649 | 63.012 | 73.143 | 1.00 | 9.30 | D |
| ATOM | 5273 | O | SER | D | 154 | 1.211 | 61.890 | 72.877 | 1.00 | 10.82 | D |
| ATOM | 5274 | N | ALA | D | 155 | 2.927 | 63.341 | 73.006 | 1.00 | 9.09 | D |
| ATOM | 5275 | CA | ALA | D | 155 | 3.911 | 62.357 | 72.585 | 1.00 | 9.10 | D |
| ATOM | 5276 | CB | ALA | D | 155 | 5.229 | 63.044 | 72.269 | 1.00 | 10.16 | D |
| ATOM | 5277 | C | ALA | D | 155 | 4.087 | 61.388 | 73.754 | 1.00 | 9.84 | D |
| ATOM | 5278 | O | ALA | D | 155 | 4.398 | 60.208 | 73.566 | 1.00 | 9.78 | D |
| ATOM | 5279 | N | LYS | D | 156 | 3.865 | 61.891 | 74.966 | 1.00 | 10.00 | D |
| ATOM | 5280 | CA | LYS | D | 156 | 4.015 | 61.067 | 76.157 | 1.00 | 11.41 | D |
| ATOM | 5281 | CB | LYS | D | 156 | 3.905 | 61.918 | 77.425 | 1.00 | 12.77 | D |
| ATOM | 5282 | CG | LYS | D | 156 | 4.241 | 61.128 | 78.686 | 1.00 | 15.30 | D |
| ATOM | 5283 | CD | LYS | D | 156 | 4.244 | 61.996 | 79.928 | 1.00 | 17.76 | D |
| ATOM | 5284 | CE | LYS | D | 156 | 4.546 | 61.160 | 81.168 | 1.00 | 19.49 | D |
| ATOM | 5285 | NZ | LYS | D | 156 | 4.476 | 61.970 | 82.413 | 1.00 | 23.92 | D |
| ATOM | 5286 | C | LYS | D | 156 | 3.023 | 59.907 | 76.225 | 1.00 | 13.22 | D |
| ATOM | 5287 | O | LYS | D | 156 | 3.396 | 58.804 | 76.616 | 1.00 | 13.78 | D |
| ATOM | 5288 | N | ASP | D | 157 | 1.764 | 60.144 | 75.864 | 1.00 | 13.97 | D |
| ATOM | 5289 | CA | ASP | D | 157 | 0.782 | 59.063 | 75.883 | 1.00 | 16.32 | D |
| ATOM | 5290 | CB | ASP | D | 157 | −0.350 | 59.350 | 76.876 | 1.00 | 18.33 | D |
| ATOM | 5291 | CG | ASP | D | 157 | −1.057 | 60.655 | 76.604 | 1.00 | 23.26 | D |
| ATOM | 5292 | OD1 | ASP | D | 157 | −1.345 | 60.948 | 75.425 | 1.00 | 24.08 | D |
| ATOM | 5293 | OD2 | ASP | D | 157 | −1.339 | 61.384 | 77.580 | 1.00 | 26.65 | D |
| ATOM | 5294 | C | ASP | D | 157 | 0.205 | 58.749 | 74.506 | 1.00 | 15.01 | D |
| ATOM | 5295 | O | ASP | D | 157 | −0.766 | 58.000 | 74.391 | 1.00 | 17.21 | D |
| ATOM | 5296 | N | ALA | D | 158 | 0.812 | 59.332 | 73.473 | 1.00 | 13.77 | D |
| ATOM | 5297 | CA | ALA | D | 158 | 0.428 | 59.116 | 72.077 | 1.00 | 14.16 | D |
| ATOM | 5298 | CB | ALA | D | 158 | 0.455 | 57.616 | 71.765 | 1.00 | 14.03 | D |
| ATOM | 5299 | C | ALA | D | 158 | −0.903 | 59.709 | 71.613 | 1.00 | 13.09 | D |
| ATOM | 5300 | O | ALA | D | 158 | −1.313 | 59.492 | 70.472 | 1.00 | 15.25 | D |
| ATOM | 5301 | N | THR | D | 159 | −1.574 | 60.462 | 72.475 | 1.00 | 12.17 | D |
| ATOM | 5302 | CA | THR | D | 159 | −2.859 | 61.036 | 72.102 | 1.00 | 12.17 | D |
| ATOM | 5303 | CB | THR | D | 159 | −3.423 | 61.924 | 73.225 | 1.00 | 14.38 | D |
| ATOM | 5304 | OG1 | THR | D | 159 | −3.564 | 61.149 | 74.421 | 1.00 | 15.60 | D |
| ATOM | 5305 | CG2 | THR | D | 159 | −4.789 | 62.478 | 72.824 | 1.00 | 16.16 | D |
| ATOM | 5306 | C | THR | D | 159 | −2.796 | 61.863 | 70.821 | 1.00 | 10.89 | D |
| ATOM | 5307 | O | THR | D | 159 | −2.006 | 62.799 | 70.711 | 1.00 | 10.68 | D |
| ATOM | 5308 | N | ASN | D | 160 | −3.635 | 61.493 | 69.858 | 1.00 | 11.49 | D |
| ATOM | 5309 | CA | ASN | D | 160 | −3.736 | 62.178 | 68.569 | 1.00 | 11.34 | D |
| ATOM | 5310 | CB | ASN | D | 160 | −4.365 | 63.566 | 68.753 | 1.00 | 11.62 | D |
| ATOM | 5311 | CG | ASN | D | 160 | −5.835 | 63.493 | 69.101 | 1.00 | 13.79 | D |
| ATOM | 5312 | OD1 | ASN | D | 160 | −6.569 | 62.677 | 68.546 | 1.00 | 14.05 | D |
| ATOM | 5313 | ND2 | ASN | D | 160 | −6.280 | 64.355 | 70.012 | 1.00 | 13.51 | D |
| ATOM | 5314 | C | ASN | D | 160 | −2.472 | 62.335 | 67.729 | 1.00 | 10.15 | D |
| ATOM | 5315 | O | ASN | D | 160 | −2.466 | 63.117 | 66.779 | 1.00 | 8.60 | D |
| ATOM | 5316 | N | VAL | D | 161 | −1.409 | 61.607 | 68.043 | 1.00 | 8.91 | D |
| ATOM | 5317 | CA | VAL | D | 161 | −0.193 | 61.751 | 67.251 | 1.00 | 8.51 | D |
| ATOM | 5318 | CB | VAL | D | 161 | 0.995 | 61.022 | 67.902 | 1.00 | 9.80 | D |
| ATOM | 5319 | CG1 | VAL | D | 161 | 2.237 | 61.160 | 67.019 | 1.00 | 8.87 | D |
| ATOM | 5320 | CG2 | VAL | D | 161 | 1.259 | 61.609 | 69.291 | 1.00 | 9.95 | D |
| ATOM | 5321 | C | VAL | D | 161 | −0.394 | 61.232 | 65.828 | 1.00 | 9.29 | D |
| ATOM | 5322 | O | VAL | D | 161 | −0.124 | 61.940 | 64.856 | 1.00 | 8.99 | D |
| ATOM | 5323 | N | ALA | D | 162 | −0.877 | 60.002 | 65.706 | 1.00 | 8.41 | D |
| ATOM | 5324 | CA | ALA | D | 162 | −1.118 | 59.413 | 64.396 | 1.00 | 8.29 | D |
| ATOM | 5325 | CB | ALA | D | 162 | −1.548 | 57.960 | 64.553 | 1.00 | 9.26 | D |
| ATOM | 5326 | C | ALA | D | 162 | −2.190 | 60.198 | 63.635 | 1.00 | 9.20 | D |
| ATOM | 5327 | O | ALA | D | 162 | −2.094 | 60.387 | 62.424 | 1.00 | 7.93 | D |
| ATOM | 5328 | N | ALA | D | 163 | −3.208 | 60.662 | 64.353 | 1.00 | 9.19 | D |
| ATOM | 5329 | CA | ALA | D | 163 | −4.291 | 61.415 | 63.731 | 1.00 | 9.53 | D |
| ATOM | 5330 | CB | ALA | D | 163 | −5.348 | 61.759 | 64.772 | 1.00 | 11.15 | D |
| ATOM | 5331 | C | ALA | D | 163 | −3.766 | 62.691 | 63.080 | 1.00 | 9.06 | D |
| ATOM | 5332 | O | ALA | D | 163 | −4.182 | 63.059 | 61.977 | 1.00 | 9.07 | D |
| ATOM | 5333 | N | ALA | D | 164 | −2.849 | 63.361 | 63.770 | 1.00 | 7.83 | D |
| ATOM | 5334 | CA | ALA | D | 164 | −2.276 | 64.601 | 63.264 | 1.00 | 9.50 | D |
| ATOM | 5335 | CB | ALA | D | 164 | −1.396 | 65.243 | 64.337 | 1.00 | 8.60 | D |
| ATOM | 5336 | C | ALA | D | 164 | −1.474 | 64.370 | 61.983 | 1.00 | 9.25 | D |
| ATOM | 5337 | O | ALA | D | 164 | −1.638 | 65.096 | 61.002 | 1.00 | 9.02 | D |
| ATOM | 5338 | N | PHE | D | 165 | −0.608 | 63.361 | 61.985 | 1.00 | 9.56 | D |
| ATOM | 5339 | CA | PHE | D | 165 | 0.197 | 63.072 | 60.802 | 1.00 | 9.68 | D |
| ATOM | 5340 | CB | PHE | D | 165 | 1.246 | 61.996 | 61.119 | 1.00 | 9.29 | D |
| ATOM | 5341 | CG | PHE | D | 165 | 2.483 | 62.538 | 61.792 | 1.00 | 9.12 | D |
| ATOM | 5342 | CD1 | PHE | D | 165 | 3.411 | 63.283 | 61.068 | 1.00 | 9.67 | D |
| ATOM | 5343 | CD2 | PHE | D | 165 | 2.705 | 62.330 | 63.149 | 1.00 | 7.94 | D |
| ATOM | 5344 | CE1 | PHE | D | 165 | 4.544 | 63.816 | 61.689 | 1.00 | 11.34 | D |
| ATOM | 5345 | CE2 | PHE | D | 165 | 3.833 | 62.856 | 63.783 | 1.00 | 10.54 | D |
| ATOM | 5346 | CZ | PHE | D | 165 | 4.753 | 63.600 | 63.052 | 1.00 | 11.76 | D |
| ATOM | 5347 | C | PHE | D | 165 | −0.663 | 62.656 | 59.614 | 1.00 | 9.88 | D |
| ATOM | 5348 | O | PHE | D | 165 | −0.396 | 63.053 | 58.478 | 1.00 | 10.31 | D |
| ATOM | 5349 | N | GLU | D | 166 | −1.705 | 61.874 | 59.878 | 1.00 | 9.44 | D |
| ATOM | 5350 | CA | GLU | D | 166 | −2.603 | 61.415 | 58.826 | 1.00 | 10.47 | D |
| ATOM | 5351 | CB | GLU | D | 166 | −3.572 | 60.376 | 59.398 | 1.00 | 10.39 | D |
| ATOM | 5352 | CG | GLU | D | 166 | −2.851 | 59.127 | 59.891 | 1.00 | 13.46 | D |
| ATOM | 5353 | CD | GLU | D | 166 | −3.683 | 58.282 | 60.847 | 1.00 | 14.20 | D |
| ATOM | 5354 | OE1 | GLU | D | 166 | −4.782 | 58.718 | 61.239 | 1.00 | 15.18 | D |
| ATOM | 5355 | OE2 | GLU | D | 166 | −3.223 | 57.181 | 61.214 | 1.00 | 14.63 | D |
| ATOM | 5356 | C | GLU | D | 166 | −3.366 | 62.585 | 58.222 | 1.00 | 9.28 | D |
| ATOM | 5357 | O | GLU | D | 166 | −3.631 | 62.618 | 57.019 | 1.00 | 9.57 | D |
| ATOM | 5358 | N | GLU | D | 167 | −3.717 | 63.549 | 59.062 | 1.00 | 9.63 | D |
| ATOM | 5359 | CA | GLU | D | 167 | −4.441 | 64.724 | 58.596 | 1.00 | 9.50 | D |
| ATOM | 5360 | CB | GLU | D | 167 | −4.802 | 65.625 | 59.775 | 1.00 | 11.96 | D |
| ATOM | 5361 | CG | GLU | D | 167 | −5.588 | 66.877 | 59.397 | 1.00 | 13.93 | D |
| ATOM | 5362 | CD | GLU | D | 167 | −6.873 | 66.559 | 58.656 | 1.00 | 16.22 | D |
| ATOM | 5363 | OE1 | GLU | D | 167 | −7.505 | 65.528 | 58.974 | 1.00 | 18.72 | D |
| ATOM | 5364 | OE2 | GLU | D | 167 | −7.260 | 67.345 | 57.765 | 1.00 | 18.82 | D |
| ATOM | 5365 | C | GLU | D | 167 | −3.570 | 65.490 | 57.608 | 1.00 | 9.29 | D |
| ATOM | 5366 | O | GLU | D | 167 | −4.069 | 66.066 | 56.646 | 1.00 | 8.28 | D |
| ATOM | 5367 | N | ALA | D | 168 | −2.262 | 65.487 | 57.850 | 1.00 | 9.17 | D |
| ATOM | 5368 | CA | ALA | D | 168 | −1.328 | 66.171 | 56.966 | 1.00 | 9.32 | D |
| ATOM | 5369 | CB | ALA | D | 168 | 0.100 | 66.001 | 57.471 | 1.00 | 10.42 | D |
| ATOM | 5370 | C | ALA | D | 168 | −1.461 | 65.594 | 55.561 | 1.00 | 10.19 | D |
| ATOM | 5371 | O | ALA | D | 168 | −1.518 | 66.335 | 54.578 | 1.00 | 9.56 | D |
| ATOM | 5372 | N | VAL | D | 169 | −1.510 | 64.268 | 55.470 | 1.00 | 10.36 | D |
| ATOM | 5373 | CA | VAL | D | 169 | −1.646 | 63.607 | 54.180 | 1.00 | 9.51 | D |
| ATOM | 5374 | CB | VAL | D | 169 | −1.569 | 62.067 | 54.320 | 1.00 | 9.80 | D |
| ATOM | 5375 | CG1 | VAL | D | 169 | −1.842 | 61.402 | 52.977 | 1.00 | 9.62 | D |
| ATOM | 5376 | CG2 | VAL | D | 169 | −0.192 | 61.661 | 54.834 | 1.00 | 9.45 | D |
| ATOM | 5377 | C | VAL | D | 169 | −2.968 | 63.998 | 53.532 | 1.00 | 10.07 | D |
| ATOM | 5378 | O | VAL | D | 169 | −3.011 | 64.285 | 52.336 | 1.00 | 9.97 | D |
| ATOM | 5379 | N | ARG | D | 170 | −4.046 | 64.011 | 54.315 | 1.00 | 9.38 | D |
| ATOM | 5380 | CA | ARG | D | 170 | −5.352 | 64.384 | 53.782 | 1.00 | 11.52 | D |
| ATOM | 5381 | CB | ARG | D | 170 | −6.421 | 64.372 | 54.884 | 1.00 | 12.49 | D |
| ATOM | 5382 | CG | ARG | D | 170 | −6.830 | 62.986 | 55.375 | 1.00 | 13.76 | D |
| ATOM | 5383 | CD | ARG | D | 170 | −7.914 | 63.093 | 56.457 | 1.00 | 16.99 | D |
| ATOM | 5384 | NE | ARG | D | 170 | −8.334 | 61.790 | 56.971 | 1.00 | 17.01 | D |
| ATOM | 5385 | CZ | ARG | D | 170 | −9.113 | 60.934 | 56.315 | 1.00 | 19.04 | D |
| ATOM | 5386 | NH1 | ARG | D | 170 | −9.572 | 61.232 | 55.106 | 1.00 | 19.15 | D |
| ATOM | 5387 | NH2 | ARG | D | 170 | −9.435 | 59.773 | 56.870 | 1.00 | 19.84 | D |
| ATOM | 5388 | C | ARG | D | 170 | −5.286 | 65.776 | 53.158 | 1.00 | 10.30 | D |
| ATOM | 5389 | O | ARG | D | 170 | −5.774 | 65.987 | 52.051 | 1.00 | 13.14 | D |
| ATOM | 5390 | N | ARG | D | 171 | −4.677 | 66.720 | 53.872 | 1.00 | 11.85 | D |
| ATOM | 5391 | CA | ARG | D | 171 | −4.554 | 68.095 | 53.387 | 1.00 | 11.09 | D |
| ATOM | 5392 | CB | ARG | D | 171 | −3.913 | 68.974 | 54.465 | 1.00 | 11.95 | D |
| ATOM | 5393 | CG | ARG | D | 171 | −4.790 | 69.150 | 55.700 | 1.00 | 14.45 | D |
| ATOM | 5394 | CD | ARG | D | 171 | −5.957 | 70.093 | 55.426 | 1.00 | 17.38 | D |
| ATOM | 5395 | NE | ARG | D | 171 | −7.038 | 69.926 | 56.394 | 1.00 | 22.92 | D |
| ATOM | 5396 | CZ | ARG | D | 171 | −8.007 | 70.815 | 56.600 | 1.00 | 25.76 | D |
| ATOM | 5397 | NH1 | ARG | D | 171 | −8.033 | 71.948 | 55.910 | 1.00 | 28.31 | D |
| ATOM | 5398 | NH2 | ARG | D | 171 | −8.962 | 70.565 | 57.488 | 1.00 | 27.03 | D |
| ATOM | 5399 | C | ARG | D | 171 | −3.750 | 68.184 | 52.091 | 1.00 | 11.43 | D |
| ATOM | 5400 | O | ARG | D | 171 | −4.072 | 68.984 | 51.212 | 1.00 | 10.60 | D |
| ATOM | 5401 | N | VAL | D | 172 | −2.704 | 67.372 | 51.966 | 1.00 | 11.33 | D |
| ATOM | 5402 | CA | VAL | D | 172 | −1.901 | 67.386 | 50.747 | 1.00 | 12.40 | D |
| ATOM | 5403 | CB | VAL | D | 172 | −0.592 | 66.585 | 50.917 | 1.00 | 11.93 | D |
| ATOM | 5404 | CG1 | VAL | D | 172 | 0.185 | 66.569 | 49.601 | 1.00 | 12.71 | D |
| ATOM | 5405 | CG2 | VAL | D | 172 | 0.258 | 67.213 | 52.017 | 1.00 | 12.48 | D |
| ATOM | 5406 | C | VAL | D | 172 | −2.707 | 66.800 | 49.588 | 1.00 | 12.50 | D |
| ATOM | 5407 | O | VAL | D | 172 | −2.751 | 67.374 | 48.499 | 1.00 | 11.70 | D |
| ATOM | 5408 | N | LEU | D | 173 | −3.363 | 65.666 | 49.823 | 1.00 | 12.90 | D |
| ATOM | 5409 | CA | LEU | D | 173 | −4.167 | 65.045 | 48.773 | 1.00 | 15.66 | D |
| ATOM | 5410 | CB | LEU | D | 173 | −4.844 | 63.773 | 49.296 | 1.00 | 15.42 | D |
| ATOM | 5411 | CG | LEU | D | 173 | −3.916 | 62.603 | 49.635 | 1.00 | 15.11 | D |
| ATOM | 5412 | CD1 | LEU | D | 173 | −4.722 | 61.481 | 50.267 | 1.00 | 15.58 | D |
| ATOM | 5413 | CD2 | LEU | D | 173 | −3.211 | 62.110 | 48.366 | 1.00 | 15.15 | D |
| ATOM | 5414 | C | LEU | D | 173 | −5.226 | 66.023 | 48.267 | 1.00 | 16.75 | D |
| ATOM | 5415 | O | LEU | D | 173 | −5.587 | 66.010 | 47.090 | 1.00 | 17.67 | D |
| ATOM | 5416 | N | ALA | D | 174 | −5.713 | 66.875 | 49.163 | 1.00 | 19.06 | D |
| ATOM | 5417 | CA | ALA | D | 174 | −6.730 | 67.858 | 48.812 | 1.00 | 21.53 | D |
| ATOM | 5418 | CB | ALA | D | 174 | −7.250 | 68.540 | 50.067 | 1.00 | 21.72 | D |
| ATOM | 5419 | C | ALA | D | 174 | −6.202 | 68.904 | 47.832 | 1.00 | 23.86 | D |
| ATOM | 5420 | O | ALA | D | 174 | −6.982 | 69.618 | 47.208 | 1.00 | 25.56 | D |
| ATOM | 5421 | N | THR | D | 175 | −4.881 | 69.000 | 47.699 | 1.00 | 25.22 | D |
| ATOM | 5422 | CA | THR | D | 175 | −4.290 | 69.972 | 46.780 | 1.00 | 26.37 | D |
| ATOM | 5423 | CB | THR | D | 175 | −2.921 | 70.473 | 47.283 | 1.00 | 26.25 | D |
| ATOM | 5424 | OG1 | THR | D | 175 | −1.966 | 69.407 | 47.217 | 1.00 | 26.91 | D |
| ATOM | 5425 | CG2 | THR | D | 175 | −3.029 | 70.968 | 48.719 | 1.00 | 24.46 | D |
| ATOM | 5426 | C | THR | D | 175 | −4.097 | 69.381 | 45.386 | 1.00 | 27.94 | D |
| ATOM | 5427 | O | THR | D | 175 | −4.470 | 68.205 | 45.176 | 1.00 | 29.42 | D |
| ATOM | 5428 | OXT | THR | D | 175 | −3.572 | 70.106 | 44.514 | 1.00 | 28.27 | D |
| TER | | | | | | | | | | | |
| HETATM | 5429 | O | HOH | | 1 | 28.829 | 34.657 | 81.286 | 1.00 | 31.30 | S |
| HETATM | 5430 | O | HOH | | 2 | 14.894 | 67.125 | 70.589 | 1.00 | 7.34 | S |
| HETATM | 5431 | O | HOH | | 3 | 36.548 | 30.734 | 88.188 | 1.00 | 3.58 | S |
| HETATM | 5432 | O | HOH | | 4 | 21.698 | 22.734 | 61.349 | 1.00 | 8.81 | S |
| HETATM | 5433 | O | HOH | | 5 | 5.987 | 77.110 | 68.606 | 1.00 | 7.53 | S |
| HETATM | 5434 | O | HOH | | 6 | 44.470 | 38.904 | 63.101 | 1.00 | 10.60 | S |
| HETATM | 5435 | O | HOH | | 7 | 25.723 | 33.215 | 85.690 | 1.00 | 6.33 | S |
| HETATM | 5436 | O | HOH | | 8 | 31.682 | 50.769 | 53.487 | 1.00 | 6.91 | S |
| HETATM | 5437 | O | HOH | | 9 | 44.009 | 43.605 | 74.252 | 1.00 | 9.63 | S |
| HETATM | 5438 | O | HOH | | 10 | 14.333 | 61.267 | 49.527 | 1.00 | 10.62 | S |
| HETATM | 5439 | O | HOH | | 11 | 0.339 | 36.416 | 75.862 | 1.00 | 12.44 | S |
| HETATM | 5440 | O | HOH | | 12 | 35.459 | 42.132 | 66.276 | 1.00 | 9.67 | S |
| HETATM | 5441 | O | HOH | | 13 | 10.219 | 47.294 | 61.872 | 1.00 | 12.42 | S |
| HETATM | 5442 | O | HOH | | 14 | 32.647 | 47.579 | 45.390 | 1.00 | 12.23 | S |
| HETATM | 5443 | O | HOH | | 15 | 4.542 | 52.698 | 58.454 | 1.00 | 12.17 | S |
| HETATM | 5444 | O | HOH | | 16 | 21.474 | 63.619 | 44.245 | 1.00 | 13.52 | S |
| HETATM | 5445 | O | HOH | | 17 | 10.514 | 61.454 | 73.037 | 1.00 | 9.68 | S |
| HETATM | 5446 | O | HOH | | 18 | 18.985 | 29.069 | 81.260 | 1.00 | 8.78 | S |
| HETATM | 5447 | O | HOH | | 19 | 19.345 | 40.090 | 74.792 | 1.00 | 12.18 | S |
| HETATM | 5448 | O | HOH | | 20 | 21.505 | 40.817 | 62.225 | 1.00 | 12.24 | S |
| HETATM | 5449 | O | HOH | | 21 | 51.353 | 53.037 | 56.261 | 1.00 | 13.85 | S |
| HETATM | 5450 | O | HOH | | 22 | 38.397 | 40.372 | 63.676 | 1.00 | 8.43 | S |
| HETATM | 5451 | O | HOH | | 23 | −1.453 | 58.099 | 68.088 | 1.00 | 13.25 | S |
| HETATM | 5452 | O | HOH | | 24 | −4.724 | 44.723 | 59.374 | 1.00 | 10.43 | S |
| HETATM | 5453 | O | HOH | | 25 | −6.386 | 50.009 | 49.584 | 1.00 | 11.22 | S |
| HETATM | 5454 | O | HOH | | 26 | 22.870 | 39.619 | 88.417 | 1.00 | 11.46 | S |
| HETATM | 5455 | O | HOH | | 27 | 21.096 | 24.654 | 83.967 | 1.00 | 18.58 | S |
| HETATM | 5456 | O | HOH | | 28 | 11.344 | 38.322 | 58.789 | 1.00 | 10.20 | S |
| HETATM | 5457 | O | HOH | | 29 | 31.815 | 44.654 | 80.076 | 1.00 | 11.18 | S |
| HETATM | 5458 | O | HOH | | 30 | 20.963 | 36.852 | 68.307 | 1.00 | 10.28 | S |
| HETATM | 5459 | O | HOH | | 31 | 2.640 | 24.609 | 63.498 | 1.00 | 9.41 | S |
| HETATM | 5460 | O | HOH | | 32 | 32.706 | 22.590 | 88.725 | 1.00 | 14.87 | S |
| HETATM | 5461 | O | HOH | | 33 | 29.368 | 18.162 | 76.026 | 1.00 | 9.70 | S |
| HETATM | 5462 | O | HOH | | 34 | 7.047 | 34.873 | 51.934 | 1.00 | 14.16 | S |
| HETATM | 5463 | O | HOH | | 35 | 13.749 | 69.257 | 66.766 | 1.00 | 13.08 | S |
| HETATM | 5464 | O | HOH | | 36 | 47.271 | 24.730 | 76.629 | 1.00 | 10.97 | S |
| HETATM | 5465 | O | HOH | | 37 | 49.306 | 36.022 | 55.516 | 1.00 | 10.71 | S |
| HETATM | 5466 | O | HOH | | 38 | −3.948 | 59.333 | 66.777 | 1.00 | 13.52 | S |
| HETATM | 5467 | O | HOH | | 39 | −3.019 | 37.748 | 67.233 | 1.00 | 12.77 | S |
| HETATM | 5468 | O | HOH | | 40 | −1.360 | 53.471 | 56.934 | 1.00 | 11.64 | S |
| HETATM | 5469 | O | HOH | | 41 | −4.718 | 74.501 | 83.245 | 1.00 | 14.60 | S |
| HETATM | 5470 | O | HOH | | 42 | 47.683 | 44.275 | 76.479 | 1.00 | 16.95 | S |
| HETATM | 5471 | O | HOH | | 43 | 19.601 | 70.489 | 75.446 | 1.00 | 14.41 | S |
| HETATM | 5472 | O | HOH | | 44 | 24.710 | 56.662 | 59.601 | 1.00 | 14.82 | S |
| HETATM | 5473 | O | HOH | | 45 | 52.609 | 29.128 | 67.488 | 1.00 | 11.92 | S |
| HETATM | 5474 | O | HOH | | 46 | 20.872 | 37.364 | 86.440 | 1.00 | 15.95 | S |
| HETATM | 5475 | O | HOH | | 47 | 7.387 | 53.386 | 61.662 | 1.00 | 10.89 | S |
| HETATM | 5476 | O | HOH | | 48 | 3.632 | 54.489 | 66.424 | 1.00 | 12.39 | S |
| HETATM | 5477 | O | HOH | | 49 | 19.705 | 21.839 | 72.990 | 1.00 | 9.68 | S |
| HETATM | 5478 | O | HOH | | 50 | 27.756 | 38.965 | 82.459 | 1.00 | 3.93 | S |
| HETATM | 5479 | O | HOH | | 51 | 46.719 | 41.933 | 74.394 | 1.00 | 10.55 | S |
| HETATM | 5480 | O | HOH | | 52 | 46.202 | 58.072 | 47.597 | 1.00 | 14.17 | S |
| HETATM | 5481 | O | HOH | | 53 | 21.419 | 51.572 | 57.609 | 1.00 | 12.59 | S |
| HETATM | 5482 | O | HOH | | 54 | 18.239 | 45.012 | 65.668 | 1.00 | 16.62 | S |
| HETATM | 5483 | O | HOH | | 55 | 36.016 | 48.305 | 46.276 | 1.00 | 14.54 | S |
| HETATM | 5484 | O | HOH | | 56 | 23.518 | 28.995 | 91.003 | 1.00 | 13.96 | S |
| HETATM | 5485 | O | HOH | | 57 | 49.266 | 32.938 | 55.212 | 1.00 | 13.25 | S |
| HETATM | 5486 | O | HOH | | 58 | 29.442 | 36.428 | 64.400 | 1.00 | 13.09 | S |
| HETATM | 5487 | O | HOH | | 59 | 45.929 | 57.104 | 59.216 | 1.00 | 11.20 | S |
| HETATM | 5488 | O | HOH | | 60 | 20.936 | 22.219 | 70.185 | 1.00 | 14.80 | S |
| HETATM | 5489 | O | HOH | | 61 | 21.309 | 22.398 | 77.082 | 1.00 | 12.12 | S |
| HETATM | 5490 | O | HOH | | 62 | −7.689 | 67.726 | 69.378 | 1.00 | 13.90 | S |
| HETATM | 5491 | O | HOH | | 63 | 51.109 | 26.395 | 67.562 | 1.00 | 14.59 | S |
| HETATM | 5492 | O | HOH | | 64 | 17.001 | 73.498 | 68.772 | 1.00 | 13.86 | S |
| HETATM | 5493 | O | HOH | | 65 | 21.720 | 74.963 | 55.506 | 1.00 | 12.35 | S |
| HETATM | 5494 | O | HOH | | 66 | 13.515 | 56.158 | 56.134 | 1.00 | 10.33 | S |
| HETATM | 5495 | O | HOH | | 67 | −3.084 | 28.842 | 59.831 | 1.00 | 9.99 | S |
| HETATM | 5496 | O | HOH | | 68 | 49.517 | 45.136 | 57.458 | 1.00 | 14.13 | S |
| HETATM | 5497 | O | HOH | | 69 | 33.535 | 32.927 | 92.926 | 1.00 | 14.88 | S |
| HETATM | 5498 | O | HOH | | 70 | 22.009 | 58.436 | 59.007 | 1.00 | 12.29 | S |
| HETATM | 5499 | O | HOH | | 71 | 28.738 | 41.582 | 69.679 | 1.00 | 15.23 | S |
| HETATM | 5500 | O | HOH | | 72 | 2.532 | 38.103 | 77.095 | 1.00 | 14.65 | S |
| HETATM | 5501 | O | HOH | | 73 | 47.462 | 46.590 | 58.604 | 1.00 | 12.49 | S |
| HETATM | 5502 | O | HOH | | 74 | 21.655 | 35.183 | 88.623 | 1.00 | 14.94 | S |
| HETATM | 5503 | O | HOH | | 75 | 40.246 | 49.512 | 88.671 | 1.00 | 20.92 | S |
| HETATM | 5504 | O | HOH | | 76 | 29.160 | 45.477 | 55.512 | 1.00 | 16.45 | S |
| HETATM | 5505 | O | HOH | | 77 | 52.220 | 27.120 | 83.328 | 1.00 | 13.67 | S |
| HETATM | 5506 | O | HOH | | 78 | 18.002 | 38.562 | 67.318 | 1.00 | 16.76 | S |
| HETATM | 5507 | O | HOH | | 79 | 17.244 | 34.916 | 62.253 | 1.00 | 11.03 | S |
| HETATM | 5508 | O | HOH | | 80 | 51.709 | 34.788 | 64.068 | 1.00 | 13.77 | S |
| HETATM | 5509 | O | HOH | | 81 | 17.787 | 40.672 | 57.657 | 1.00 | 17.70 | S |
| HETATM | 5510 | O | HOH | | 82 | 36.988 | 15.110 | 85.043 | 1.00 | 13.31 | S |
| HETATM | 5511 | O | HOH | | 83 | 4.168 | 16.802 | 83.124 | 1.00 | 8.98 | S |
| HETATM | 5512 | O | HOH | | 84 | 50.210 | 38.055 | 82.136 | 1.00 | 14.93 | S |
| HETATM | 5513 | O | HOH | | 85 | 52.503 | 47.063 | 57.964 | 1.00 | 18.26 | S |
| HETATM | 5514 | O | HOH | | 86 | 30.447 | 25.368 | 66.410 | 1.00 | 13.78 | S |
| HETATM | 5515 | O | HOH | | 87 | 8.519 | 60.362 | 71.677 | 1.00 | 17.17 | S |
| HETATM | 5516 | O | HOH | | 88 | −2.631 | 23.246 | 76.472 | 1.00 | 22.14 | S |
| HETATM | 5517 | O | HOH | | 89 | 25.033 | 22.481 | 51.666 | 1.00 | 16.48 | S |
| HETATM | 5518 | O | HOH | | 90 | 28.688 | 28.052 | 63.418 | 1.00 | 14.04 | S |
| HETATM | 5519 | O | HOH | | 91 | 25.832 | 25.738 | 57.642 | 1.00 | 14.77 | S |
| HETATM | 5520 | O | HOH | | 92 | 13.654 | 43.420 | 56.402 | 1.00 | 15.24 | S |
| HETATM | 5521 | O | HOH | | 93 | 21.835 | 20.566 | 48.488 | 1.00 | 15.78 | S |
| HETATM | 5522 | O | HOH | | 94 | 48.755 | 27.801 | 93.630 | 1.00 | 28.75 | S |
| HETATM | 5523 | O | HOH | | 95 | 29.524 | 31.256 | 90.447 | 1.00 | 17.53 | S |
| HETATM | 5524 | O | HOH | | 96 | −1.219 | 20.866 | 86.704 | 1.00 | 14.72 | S |
| HETATM | 5525 | O | HOH | | 97 | 52.942 | 28.878 | 85.259 | 1.00 | 17.90 | S |
| HETATM | 5526 | O | HOH | | 98 | 33.733 | 44.487 | 78.244 | 1.00 | 14.51 | S |
| HETATM | 5527 | O | HOH | | 99 | 10.822 | 49.779 | 65.169 | 1.00 | 23.36 | S |
| HETATM | 5528 | O | HOH | | 100 | 19.920 | 36.281 | 56.591 | 1.00 | 18.49 | S |
| HETATM | 5529 | O | HOH | | 101 | 28.665 | 55.870 | 38.409 | 1.00 | 16.75 | S |
| HETATM | 5530 | O | HOH | | 102 | 28.187 | 27.210 | 58.584 | 1.00 | 13.50 | S |
| HETATM | 5531 | O | HOH | | 103 | 35.219 | 38.680 | 61.940 | 1.00 | 15.05 | S |
| HETATM | 5532 | O | HOH | | 104 | 30.325 | 29.296 | 61.394 | 1.00 | 17.05 | S |
| HETATM | 5533 | O | HOH | | 105 | 18.007 | 29.766 | 87.069 | 1.00 | 21.84 | S |
| HETATM | 5534 | O | HOH | | 106 | 24.862 | 38.549 | 79.595 | 1.00 | 14.15 | S |
| HETATM | 5535 | O | HOH | | 107 | 39.137 | 44.596 | 70.525 | 1.00 | 19.53 | S |
| HETATM | 5536 | O | HOH | | 108 | 12.420 | 65.431 | 49.806 | 1.00 | 18.72 | S |
| HETATM | 5537 | O | HOH | | 109 | 35.508 | 40.778 | 57.761 | 1.00 | 16.43 | S |
| HETATM | 5538 | O | HOH | | 110 | −1.129 | 82.097 | 68.378 | 1.00 | 15.44 | S |
| HETATM | 5539 | O | HOH | | 111 | 25.014 | 48.103 | 54.322 | 1.00 | 19.57 | S |
| HETATM | 5540 | O | HOH | | 112 | 45.066 | 19.504 | 79.646 | 1.00 | 23.40 | S |
| HETATM | 5541 | O | HOH | | 113 | 18.842 | 46.905 | 53.340 | 1.00 | 17.60 | S |
| HETATM | 5542 | O | HOH | | 114 | 27.366 | 34.090 | 56.443 | 1.00 | 20.06 | S |
| HETATM | 5543 | O | HOH | | 115 | 29.083 | 26.613 | 88.014 | 1.00 | 17.98 | S |
| HETATM | 5544 | O | HOH | | 116 | 23.497 | 50.679 | 50.366 | 1.00 | 21.62 | S |
| HETATM | 5545 | O | HOH | | 117 | 8.867 | 85.265 | 61.217 | 1.00 | 17.10 | S |
| HETATM | 5546 | O | HOH | | 118 | −1.071 | 36.203 | 51.903 | 1.00 | 23.64 | S |
| HETATM | 5547 | O | HOH | | 119 | 15.951 | 30.766 | 51.558 | 1.00 | 19.01 | S |
| HETATM | 5548 | O | HOH | | 120 | 22.394 | 18.805 | 80.727 | 1.00 | 11.54 | S |
| HETATM | 5549 | O | HOH | | 121 | 29.531 | 44.256 | 78.321 | 1.00 | 16.18 | S |
| HETATM | 5550 | O | HOH | | 122 | 19.738 | 21.700 | 79.314 | 1.00 | 17.14 | S |
| HETATM | 5551 | O | HOH | | 123 | 14.257 | 46.702 | 72.680 | 1.00 | 18.26 | S |
| HETATM | 5552 | O | HOH | | 124 | 24.026 | 72.882 | 63.077 | 1.00 | 19.77 | S |
| HETATM | 5553 | O | HOH | | 125 | 17.757 | 65.433 | 68.253 | 1.00 | 14.58 | S |
| HETATM | 5554 | O | HOH | | 126 | −1.269 | 39.312 | 75.760 | 1.00 | 21.97 | S |
| HETATM | 5555 | O | HOH | | 127 | −8.828 | 56.854 | 54.666 | 1.00 | 14.81 | S |
| HETATM | 5556 | O | HOH | | 128 | 22.027 | 39.778 | 71.208 | 1.00 | 16.95 | S |
| HETATM | 5557 | O | HOH | | 129 | 8.280 | 74.892 | 47.906 | 1.00 | 17.57 | S |
| HETATM | 5558 | O | HOH | | 130 | 19.001 | 80.255 | 69.828 | 1.00 | 18.56 | S |
| HETATM | 5559 | O | HOH | | 131 | 49.515 | 35.643 | 72.772 | 1.00 | 16.97 | S |
| HETATM | 5560 | O | HOH | | 132 | 36.933 | 24.893 | 95.516 | 1.00 | 20.00 | S |
| HETATM | 5561 | O | HOH | | 133 | 11.569 | 33.684 | 83.023 | 1.00 | 23.09 | S |
| HETATM | 5562 | O | HOH | | 134 | 6.399 | 62.280 | 44.852 | 1.00 | 25.36 | S |
| HETATM | 5563 | O | HOH | | 135 | −6.484 | 51.878 | 47.230 | 1.00 | 19.95 | S |
| HETATM | 5564 | O | HOH | | 136 | 41.900 | 24.969 | 92.173 | 1.00 | 14.97 | S |
| HETATM | 5565 | O | HOH | | 137 | 16.916 | 60.808 | 60.789 | 1.00 | 21.51 | S |
| HETATM | 5566 | O | HOH | | 138 | −0.070 | 46.145 | 71.399 | 1.00 | 23.13 | S |
| HETATM | 5567 | O | HOH | | 139 | −8.603 | 38.745 | 62.303 | 1.00 | 14.56 | S |
| HETATM | 5568 | O | HOH | | 140 | 27.509 | 29.500 | 91.565 | 1.00 | 16.31 | S |
| HETATM | 5569 | O | HOH | | 141 | 28.355 | 54.699 | 68.996 | 1.00 | 12.38 | S |
| HETATM | 5570 | O | HOH | | 142 | 41.558 | 41.533 | 98.083 | 1.00 | 23.54 | S |
| HETATM | 5571 | O | HOH | | 143 | −1.833 | 51.027 | 57.896 | 1.00 | 14.27 | S |
| HETATM | 5572 | O | HOH | | 144 | 36.666 | 23.727 | 60.947 | 1.00 | 25.59 | S |
| HETATM | 5573 | O | HOH | | 145 | 37.619 | 54.738 | 40.291 | 1.00 | 24.39 | S |
| HETATM | 5574 | O | HOH | | 146 | 22.446 | 22.687 | 86.512 | 1.00 | 13.31 | S |
| HETATM | 5575 | O | HOH | | 147 | 42.464 | 64.174 | 64.419 | 1.00 | 16.58 | S |
| HETATM | 5576 | O | HOH | | 148 | 44.676 | 29.617 | 56.466 | 1.00 | 12.58 | S |
| HETATM | 5577 | O | HOH | | 149 | 6.166 | 85.579 | 55.142 | 1.00 | 14.88 | S |
| HETATM | 5578 | O | HOH | | 150 | −0.696 | 29.886 | 73.715 | 1.00 | 16.43 | S |
| HETATM | 5579 | O | HOH | | 151 | 3.845 | 85.145 | 66.129 | 1.00 | 20.01 | S |
| HETATM | 5580 | O | HOH | | 152 | 32.726 | 46.645 | 61.990 | 1.00 | 14.96 | S |
| HETATM | 5581 | O | HOH | | 153 | −1.790 | 55.045 | 45.799 | 1.00 | 19.28 | S |
| HETATM | 5582 | O | HOH | | 154 | 5.586 | 49.747 | 52.976 | 1.00 | 17.40 | S |
| HETATM | 5583 | O | HOH | | 155 | 23.215 | 72.592 | 51.941 | 1.00 | 19.45 | S |
| HETATM | 5584 | O | HOH | | 156 | 46.563 | 43.947 | 90.394 | 1.00 | 20.92 | S |
| HETATM | 5585 | O | HOH | | 157 | 15.861 | 43.917 | 58.031 | 1.00 | 16.75 | S |
| HETATM | 5586 | O | HOH | | 158 | −3.600 | 42.875 | 66.510 | 1.00 | 18.14 | S |
| HETATM | 5587 | O | HOH | | 159 | 40.034 | 63.873 | 66.500 | 1.00 | 15.98 | S |
| HETATM | 5588 | O | HOH | | 160 | 28.284 | 34.158 | 60.004 | 1.00 | 16.77 | S |
| HETATM | 5589 | O | HOH | | 161 | 14.476 | 66.855 | 79.021 | 1.00 | 17.89 | S |
| HETATM | 5590 | O | HOH | | 162 | 30.280 | 47.156 | 59.458 | 1.00 | 27.11 | S |
| HETATM | 5591 | O | HOH | | 163 | −0.701 | 50.317 | 62.978 | 1.00 | 21.48 | S |
| HETATM | 5592 | O | HOH | | 164 | 42.364 | 37.331 | 59.769 | 1.00 | 23.14 | S |
| HETATM | 5593 | O | HOH | | 165 | 6.628 | 72.981 | 77.056 | 1.00 | 18.71 | S |
| HETATM | 5594 | O | HOH | | 166 | 38.440 | 20.001 | 92.283 | 1.00 | 18.54 | S |
| HETATM | 5595 | O | HOH | | 167 | 29.647 | 47.678 | 68.505 | 1.00 | 25.79 | S |
| HETATM | 5596 | O | HOH | | 168 | 29.185 | 23.604 | 68.874 | 1.00 | 23.88 | S |
| HETATM | 5597 | O | HOH | | 169 | 2.381 | 53.480 | 51.367 | 1.00 | 22.63 | S |
| HETATM | 5598 | O | HOH | | 170 | 23.304 | 77.069 | 56.487 | 1.00 | 23.60 | S |
| HETATM | 5599 | O | HOH | | 171 | 34.739 | 43.897 | 89.399 | 1.00 | 17.73 | S |
| HETATM | 5600 | O | HOH | | 172 | 12.766 | 58.979 | 49.205 | 1.00 | 21.12 | S |
| HETATM | 5601 | O | HOH | | 173 | 27.144 | 51.295 | 42.750 | 1.00 | 16.25 | S |
| HETATM | 5602 | O | HOH | | 174 | −4.449 | 72.966 | 56.154 | 1.00 | 19.73 | S |
| HETATM | 5603 | O | HOH | | 175 | 5.038 | 36.218 | 53.060 | 1.00 | 14.13 | S |
| HETATM | 5604 | O | HOH | | 176 | 3.676 | 27.902 | 82.717 | 1.00 | 15.03 | S |
| HETATM | 5605 | O | HOH | | 177 | 8.922 | 79.152 | 73.577 | 1.00 | 22.77 | S |
| HETATM | 5606 | O | HOH | | 178 | 0.610 | 83.571 | 66.844 | 1.00 | 16.37 | S |
| HETATM | 5607 | O | HOH | | 179 | 13.674 | 79.797 | 65.748 | 1.00 | 22.95 | S |
| HETATM | 5608 | O | HOH | | 180 | 33.505 | 51.128 | 76.851 | 1.00 | 18.14 | S |
| HETATM | 5609 | O | HOH | | 181 | 9.091 | 19.130 | 66.122 | 1.00 | 19.54 | S |
| HETATM | 5610 | O | HOH | | 182 | 21.029 | 67.447 | 67.390 | 1.00 | 21.43 | S |
| HETATM | 5611 | O | HOH | | 183 | 26.009 | 50.558 | 70.105 | 1.00 | 25.55 | S |
| HETATM | 5612 | O | HOH | | 184 | −1.363 | 47.145 | 56.428 | 1.00 | 19.88 | S |
| HETATM | 5613 | O | HOH | | 185 | 40.559 | 34.611 | 59.194 | 1.00 | 18.86 | S |
| HETATM | 5614 | O | HOH | | 186 | 12.950 | 60.604 | 71.667 | 1.00 | 25.48 | S |
| HETATM | 5615 | O | HOH | | 187 | 43.401 | 45.483 | 65.048 | 1.00 | 24.56 | S |
| HETATM | 5616 | O | HOH | | 188 | 8.267 | 74.630 | 75.000 | 1.00 | 25.27 | S |
| HETATM | 5617 | O | HOH | | 189 | 24.523 | 51.171 | 62.933 | 1.00 | 24.97 | S |
| HETATM | 5618 | O | HOH | | 190 | 5.019 | 30.932 | 79.896 | 1.00 | 18.54 | S |
| HETATM | 5619 | O | HOH | | 191 | 0.046 | 69.944 | 86.227 | 1.00 | 26.88 | S |
| HETATM | 5620 | O | HOH | | 192 | 28.512 | 15.341 | 77.931 | 1.00 | 22.51 | S |
| HETATM | 5621 | O | HOH | | 193 | 7.638 | 52.691 | 55.959 | 1.00 | 18.43 | S |
| HETATM | 5622 | O | HOH | | 194 | −1.970 | 84.174 | 69.763 | 1.00 | 36.46 | S |
| HETATM | 5623 | O | HOH | | 195 | 56.255 | 26.574 | 82.969 | 1.00 | 24.71 | S |
| HETATM | 5624 | O | HOH | | 196 | 20.609 | 80.181 | 54.968 | 1.00 | 18.58 | S |
| HETATM | 5625 | O | HOH | | 197 | 12.778 | 28.691 | 84.479 | 1.00 | 22.09 | S |
| HETATM | 5626 | O | HOH | | 198 | 15.857 | 41.922 | 79.710 | 1.00 | 15.99 | S |
| HETATM | 5627 | O | HOH | | 199 | −1.519 | 25.453 | 78.806 | 1.00 | 21.50 | S |
| HETATM | 5628 | O | HOH | | 200 | 5.381 | 26.733 | 52.098 | 1.00 | 24.23 | S |
| HETATM | 5629 | O | HOH | | 201 | 44.275 | 42.799 | 58.266 | 1.00 | 19.65 | S |
| HETATM | 5630 | O | HOH | | 202 | 14.030 | 48.110 | 63.753 | 1.00 | 20.70 | S |
| HETATM | 5631 | O | HOH | | 203 | 12.975 | 78.412 | 70.350 | 1.00 | 21.86 | S |
| HETATM | 5632 | O | HOH | | 204 | 21.954 | 78.291 | 61.631 | 1.00 | 17.21 | S |
| HETATM | 5633 | O | HOH | | 205 | 9.743 | 29.968 | 84.165 | 1.00 | 24.51 | S |
| HETATM | 5634 | O | HOH | | 206 | 24.434 | 41.048 | 56.105 | 1.00 | 21.00 | S |
| HETATM | 5635 | O | HOH | | 207 | 44.757 | 32.895 | 97.110 | 1.00 | 23.48 | S |
| HETATM | 5636 | O | HOH | | 208 | 37.311 | 38.765 | 57.704 | 1.00 | 27.21 | S |
| HETATM | 5637 | O | HOH | | 209 | 0.748 | 51.999 | 53.594 | 1.00 | 20.55 | S |
| HETATM | 5638 | O | HOH | | 210 | 35.703 | 39.476 | 91.233 | 1.00 | 24.06 | S |
| HETATM | 5639 | O | HOH | | 211 | 15.409 | 41.497 | 52.105 | 1.00 | 29.40 | S |
| HETATM | 5640 | O | HOH | | 212 | 6.497 | 36.123 | 80.042 | 1.00 | 20.82 | S |
| HETATM | 5641 | O | HOH | | 213 | 41.523 | 19.611 | 88.260 | 1.00 | 27.82 | S |
| HETATM | 5642 | O | HOH | | 214 | 39.723 | 45.731 | 63.695 | 1.00 | 27.84 | S |
| HETATM | 5643 | O | HOH | | 215 | 37.947 | 47.991 | 48.023 | 1.00 | 22.54 | S |
| HETATM | 5644 | O | HOH | | 216 | 42.726 | 54.269 | 68.186 | 1.00 | 23.43 | S |
| HETATM | 5645 | O | HOH | | 217 | 9.198 | 28.441 | 51.708 | 1.00 | 24.03 | S |
| HETATM | 5646 | O | HOH | | 218 | 2.143 | 64.057 | 82.410 | 1.00 | 20.93 | S |
| HETATM | 5647 | O | HOH | | 219 | −2.391 | 53.020 | 62.407 | 1.00 | 19.36 | S |
| HETATM | 5648 | O | HOH | | 220 | 16.874 | 18.569 | 63.035 | 1.00 | 22.49 | S |
| HETATM | 5649 | O | HOH | | 221 | −8.224 | 65.148 | 50.858 | 1.00 | 19.68 | S |
| HETATM | 5650 | O | HOH | | 222 | 17.628 | 59.426 | 43.673 | 1.00 | 23.45 | S |
| HETATM | 5651 | O | HOH | | 223 | 44.064 | 61.920 | 66.462 | 1.00 | 23.88 | S |
| HETATM | 5652 | O | HOH | | 224 | −5.161 | 59.162 | 69.777 | 1.00 | 24.83 | S |
| HETATM | 5653 | O | HOH | | 225 | 19.082 | 34.331 | 55.155 | 1.00 | 35.54 | S |
| HETATM | 5654 | O | HOH | | 226 | 21.546 | 36.590 | 80.426 | 1.00 | 16.16 | S |
| HETATM | 5655 | O | HOH | | 227 | 27.765 | 50.158 | 83.255 | 1.00 | 24.57 | S |
| HETATM | 5656 | O | HOH | | 228 | 52.768 | 32.251 | 72.743 | 1.00 | 25.55 | S |
| HETATM | 5657 | O | HOH | | 229 | 29.756 | 22.974 | 92.444 | 1.00 | 25.10 | S |
| HETATM | 5658 | O | HOH | | 230 | 44.138 | 56.711 | 40.909 | 1.00 | 27.13 | S |
| HETATM | 5659 | O | HOH | | 231 | 2.096 | 55.512 | 68.803 | 1.00 | 21.31 | S |
| HETATM | 5660 | O | HOH | | 232 | 26.080 | 67.886 | 42.788 | 1.00 | 20.62 | S |
| HETATM | 5661 | O | HOH | | 233 | 3.595 | 41.081 | 81.052 | 1.00 | 24.13 | S |
| HETATM | 5662 | O | HOH | | 234 | 23.136 | 60.475 | 63.225 | 1.00 | 19.55 | S |
| HETATM | 5663 | O | HOH | | 235 | 6.104 | 73.224 | 48.151 | 1.00 | 24.80 | S |
| HETATM | 5664 | O | HOH | | 236 | −9.690 | 63.411 | 52.866 | 1.00 | 21.97 | S |
| HETATM | 5665 | O | HOH | | 237 | 14.809 | 58.360 | 45.391 | 1.00 | 20.23 | S |
| HETATM | 5666 | O | HOH | | 238 | 25.892 | 27.244 | 53.371 | 1.00 | 24.21 | S |
| HETATM | 5667 | O | HOH | | 239 | 23.717 | 54.900 | 63.465 | 1.00 | 22.41 | S |
| HETATM | 5668 | O | HOH | | 240 | −9.775 | 44.047 | 59.390 | 1.00 | 23.32 | S |
| HETATM | 5669 | O | HOH | | 241 | 27.775 | 37.444 | 76.558 | 1.00 | 25.69 | S |
| HETATM | 5670 | O | HOH | | 242 | 7.772 | 68.659 | 79.084 | 1.00 | 31.77 | S |
| HETATM | 5671 | O | HOH | | 243 | 23.176 | 60.530 | 66.020 | 1.00 | 26.03 | S |
| HETATM | 5672 | O | HOH | | 244 | 28.938 | 47.174 | 63.906 | 1.00 | 25.35 | S |
| HETATM | 5673 | O | HOH | | 245 | 26.441 | 39.630 | 77.665 | 1.00 | 18.28 | S |
| HETATM | 5674 | O | HOH | | 246 | 20.342 | 81.406 | 62.138 | 1.00 | 22.59 | S |
| HETATM | 5675 | O | HOH | | 247 | 36.619 | 26.761 | 97.518 | 1.00 | 26.80 | S |
| HETATM | 5676 | O | HOH | | 248 | 50.254 | 41.421 | 85.825 | 1.00 | 38.33 | S |
| HETATM | 5677 | O | HOH | | 249 | 18.561 | 47.031 | 75.934 | 1.00 | 16.30 | S |
| HETATM | 5678 | O | HOH | | 250 | 47.370 | 42.777 | 96.665 | 1.00 | 19.68 | S |
| HETATM | 5679 | O | HOH | | 251 | 14.953 | 54.410 | 51.020 | 1.00 | 22.19 | S |
| HETATM | 5680 | O | HOH | | 252 | 38.752 | 23.539 | 94.546 | 1.00 | 35.70 | S |
| HETATM | 5681 | O | HOH | | 253 | 36.064 | 41.049 | 62.348 | 1.00 | 24.35 | S |
| HETATM | 5682 | O | HOH | | 254 | 50.876 | 39.105 | 92.957 | 1.00 | 20.69 | S |
| HETATM | 5683 | O | HOH | | 255 | 36.875 | 18.702 | 71.313 | 1.00 | 25.58 | S |
| HETATM | 5684 | O | HOH | | 256 | 56.303 | 31.767 | 89.740 | 1.00 | 32.25 | S |
| HETATM | 5685 | O | HOH | | 257 | 1.303 | 57.081 | 66.839 | 1.00 | 21.48 | S |
| HETATM | 5686 | O | HOH | | 258 | 13.580 | 50.846 | 49.870 | 1.00 | 25.67 | S |
| HETATM | 5687 | O | HOH | | 259 | 22.066 | 22.168 | 66.167 | 1.00 | 27.03 | S |
| HETATM | 5688 | O | HOH | | 260 | 37.831 | 21.326 | 95.426 | 1.00 | 28.14 | S |
| HETATM | 5689 | O | HOH | | 261 | 43.561 | 47.360 | 47.362 | 1.00 | 22.65 | S |
| HETATM | 5690 | O | HOH | | 262 | 0.763 | 82.340 | 79.922 | 1.00 | 28.20 | S |
| HETATM | 5691 | O | HOH | | 263 | 2.130 | 43.961 | 52.609 | 1.00 | 28.87 | S |
| HETATM | 5692 | O | HOH | | 264 | 44.868 | 41.236 | 61.947 | 1.00 | 15.21 | S |
| HETATM | 5693 | O | HOH | | 265 | 53.302 | 36.983 | 93.284 | 1.00 | 20.51 | S |
| HETATM | 5694 | O | HOH | | 266 | −1.750 | 24.987 | 54.049 | 1.00 | 23.40 | S |
| HETATM | 5695 | O | HOH | | 267 | 52.220 | 31.347 | 81.078 | 1.00 | 20.98 | S |
| HETATM | 5696 | O | HOH | | 268 | 48.410 | 51.313 | 60.017 | 1.00 | 26.46 | S |
| HETATM | 5697 | O | HOH | | 269 | 25.583 | 40.559 | 81.430 | 1.00 | 28.57 | S |
| HETATM | 5698 | O | HOH | | 270 | 34.451 | 70.116 | 45.724 | 1.00 | 27.39 | S |
| HETATM | 5699 | O | HOH | | 271 | 13.755 | 68.558 | 50.374 | 1.00 | 26.09 | S |
| HETATM | 5700 | O | HOH | | 272 | −9.651 | 39.954 | 60.007 | 1.00 | 24.09 | S |
| HETATM | 5701 | O | HOH | | 273 | 0.744 | 53.359 | 72.498 | 1.00 | 30.45 | S |
| HETATM | 5702 | O | HOH | | 274 | 10.523 | 50.642 | 67.725 | 1.00 | 25.01 | S |
| HETATM | 5703 | O | HOH | | 275 | 13.492 | 15.552 | 59.512 | 1.00 | 28.14 | S |
| HETATM | 5704 | O | HOH | | 276 | 51.558 | 20.179 | 85.096 | 1.00 | 20.83 | S |
| HETATM | 5705 | O | HOH | | 277 | 33.401 | 40.829 | 55.969 | 1.00 | 29.38 | S |
| HETATM | 5706 | O | HOH | | 278 | −2.598 | 41.607 | 68.817 | 1.00 | 24.21 | S |
| HETATM | 5707 | O | HOH | | 279 | 1.365 | 31.947 | 49.953 | 1.00 | 27.49 | S |
| HETATM | 5708 | O | HOH | | 280 | 34.930 | 17.183 | 72.328 | 1.00 | 20.72 | S |
| HETATM | 5709 | O | HOH | | 281 | 47.408 | 22.669 | 92.160 | 1.00 | 25.08 | S |
| HETATM | 5710 | O | HOH | | 282 | −9.644 | 71.497 | 63.846 | 1.00 | 27.67 | S |
| HETATM | 5711 | O | HOH | | 283 | 52.446 | 50.418 | 55.575 | 1.00 | 24.15 | S |
| HETATM | 5712 | O | HOH | | 284 | 11.711 | 54.879 | 51.563 | 1.00 | 23.56 | S |
| HETATM | 5713 | O | HOH | | 285 | 13.034 | 51.029 | 67.208 | 1.00 | 26.49 | S |
| HETATM | 5714 | O | HOH | | 286 | 31.872 | 28.557 | 94.720 | 1.00 | 33.35 | S |
| HETATM | 5715 | O | HOH | | 287 | 28.604 | 63.201 | 39.464 | 1.00 | 30.78 | S |
| HETATM | 5716 | O | HOH | | 288 | 30.193 | 75.330 | 65.299 | 1.00 | 28.11 | S |
| HETATM | 5717 | O | HOH | | 289 | 29.093 | 25.375 | 64.052 | 1.00 | 11.74 | S |
| HETATM | 5718 | O | HOH | | 290 | −1.896 | 21.706 | 74.493 | 1.00 | 32.19 | S |
| HETATM | 5719 | O | HOH | | 291 | 21.394 | 20.638 | 74.945 | 1.00 | 14.73 | S |
| HETATM | 5720 | O | HOH | | 292 | 14.012 | 63.656 | 48.034 | 1.00 | 15.45 | S |
| HETATM | 5721 | O | HOH | | 293 | 29.186 | 62.465 | 36.827 | 1.00 | 21.69 | S |
| HETATM | 5722 | O | HOH | | 294 | 21.703 | 74.908 | 52.461 | 1.00 | 21.48 | S |
| HETATM | 5723 | O | HOH | | 295 | 25.773 | 73.453 | 50.655 | 1.00 | 23.46 | S |
| HETATM | 5724 | O | HOH | | 296 | −3.330 | 35.069 | 71.964 | 1.00 | 21.77 | S |
| HETATM | 5725 | O | HOH | | 297 | 36.726 | 14.111 | 87.531 | 1.00 | 25.01 | S |
| HETATM | 5726 | O | HOH | | 298 | 16.628 | 47.788 | 64.328 | 1.00 | 23.38 | S |
| HETATM | 5727 | O | HOH | | 299 | 42.327 | 21.255 | 70.457 | 1.00 | 21.24 | S |
| HETATM | 5728 | O | HOH | | 300 | 51.552 | 37.785 | 79.551 | 1.00 | 23.00 | S |
| HETATM | 5729 | O | HOH | | 301 | −5.879 | 44.744 | 61.857 | 1.00 | 18.75 | S |
| HETATM | 5730 | O | HOH | | 302 | 6.407 | 86.029 | 52.387 | 1.00 | 22.10 | S |
| HETATM | 5731 | O | HOH | | 303 | 17.963 | 36.089 | 86.951 | 1.00 | 33.28 | S |
| HETATM | 5732 | O | HOH | | 304 | 28.126 | 33.999 | 91.114 | 1.00 | 21.98 | S |
| HETATM | 5733 | O | HOH | | 305 | 11.246 | 14.119 | 58.992 | 1.00 | 35.99 | S |
| HETATM | 5734 | O | HOH | | 306 | 19.936 | 18.154 | 81.797 | 1.00 | 23.66 | S |
| HETATM | 5735 | O | HOH | | 307 | 43.546 | 26.977 | 92.104 | 1.00 | 17.98 | S |
| HETATM | 5736 | O | HOH | | 308 | 32.803 | 23.753 | 65.934 | 1.00 | 20.17 | S |
| HETATM | 5737 | O | HOH | | 309 | 46.424 | 37.543 | 65.712 | 1.00 | 21.81 | S |
| HETATM | 5738 | O | HOH | | 310 | 11.745 | 41.198 | 50.688 | 1.00 | 28.03 | S |
| HETATM | 5739 | O | HOH | | 311 | 21.913 | 46.470 | 53.162 | 1.00 | 22.80 | S |
| HETATM | 5740 | O | HOH | | 312 | 50.903 | 39.014 | 77.153 | 1.00 | 25.11 | S |
| HETATM | 5741 | O | HOH | | 313 | 43.190 | 30.581 | 97.327 | 1.00 | 23.89 | S |
| HETATM | 5742 | O | HOH | | 314 | 0.982 | 70.818 | 49.368 | 1.00 | 21.77 | S |
| HETATM | 5743 | O | HOH | | 315 | 5.847 | 22.972 | 58.148 | 1.00 | 24.85 | S |
| HETATM | 5744 | O | HOH | | 316 | −1.147 | 82.904 | 75.040 | 1.00 | 35.65 | S |
| HETATM | 5745 | O | HOH | | 317 | 19.189 | 42.747 | 69.079 | 1.00 | 28.27 | S |
| HETATM | 5746 | O | HOH | | 318 | 2.299 | 69.812 | 47.378 | 1.00 | 29.76 | S |
| HETATM | 5747 | O | HOH | | 319 | 40.212 | 44.455 | 96.186 | 1.00 | 24.85 | S |
| HETATM | 5748 | O | HOH | | 320 | −0.568 | 33.690 | 50.845 | 1.00 | 22.12 | S |
| HETATM | 5749 | O | HOH | | 321 | 17.935 | 46.702 | 69.560 | 1.00 | 25.28 | S |
| HETATM | 5750 | O | HOH | | 322 | 14.389 | 13.032 | 78.443 | 1.00 | 26.73 | S |
| HETATM | 5751 | O | HOH | | 323 | 22.502 | 44.439 | 75.563 | 1.00 | 30.32 | S |
| HETATM | 5752 | O | HOH | | 324 | −3.540 | 56.142 | 57.995 | 1.00 | 21.37 | S |
| HETATM | 5753 | O | HOH | | 325 | 45.175 | 43.211 | 66.691 | 1.00 | 19.16 | S |
| HETATM | 5754 | O | HOH | | 326 | −7.808 | 67.937 | 74.427 | 1.00 | 28.15 | S |
| HETATM | 5755 | O | HOH | | 327 | 25.064 | 52.650 | 67.123 | 1.00 | 24.12 | S |
| HETATM | 5756 | O | HOH | | 328 | −0.743 | 19.897 | 71.602 | 1.00 | 22.10 | S |
| HETATM | 5757 | O | HOH | | 329 | 49.235 | 32.102 | 52.850 | 1.00 | 26.29 | S |
| HETATM | 5758 | O | HOH | | 330 | 23.115 | 22.342 | 68.724 | 1.00 | 28.32 | S |
| HETATM | 5759 | O | HOH | | 331 | −9.601 | 75.626 | 62.579 | 1.00 | 23.47 | S |
| HETATM | 5760 | O | HOH | | 332 | 25.119 | 22.630 | 54.347 | 1.00 | 29.27 | S |
| HETATM | 5761 | O | HOH | | 333 | −4.880 | 69.059 | 78.885 | 1.00 | 29.45 | S |
| HETATM | 5762 | O | HOH | | 334 | 6.884 | 51.220 | 57.935 | 1.00 | 23.35 | S |
| HETATM | 5763 | O | HOH | | 335 | 49.667 | 39.586 | 95.334 | 1.00 | 24.75 | S |
| HETATM | 5764 | O | HOH | | 336 | 12.045 | 81.677 | 64.086 | 1.00 | 29.15 | S |
| HETATM | 5765 | O | HOH | | 337 | 45.882 | 17.214 | 82.759 | 1.00 | 27.57 | S |
| HETATM | 5766 | O | HOH | | 338 | 24.978 | 23.319 | 58.978 | 1.00 | 25.06 | S |
| HETATM | 5767 | O | HOH | | 339 | −6.751 | 62.469 | 61.088 | 1.00 | 21.69 | S |
| HETATM | 5768 | O | HOH | | 340 | 40.334 | 45.738 | 72.689 | 1.00 | 27.32 | S |
| HETATM | 5769 | O | HOH | | 341 | 26.737 | 46.359 | 57.061 | 1.00 | 27.50 | S |
| HETATM | 5770 | O | HOH | | 342 | 37.021 | 14.773 | 77.748 | 1.00 | 29.40 | S |
| HETATM | 5771 | O | HOH | | 343 | −3.434 | 50.880 | 59.889 | 1.00 | 23.34 | S |
| HETATM | 5772 | O | HOH | | 344 | 43.143 | 43.618 | 53.649 | 1.00 | 34.81 | S |
| HETATM | 5773 | O | HOH | | 345 | 51.445 | 26.004 | 71.370 | 1.00 | 23.17 | S |
| HETATM | 5774 | O | HOH | | 346 | 53.189 | 29.774 | 71.353 | 1.00 | 30.50 | S |
| HETATM | 5775 | O | HOH | | 347 | 28.351 | 69.248 | 44.218 | 1.00 | 28.54 | S |
| HETATM | 5776 | O | HOH | | 348 | 23.282 | 18.475 | 47.796 | 1.00 | 29.02 | S |
| HETATM | 5777 | O | HOH | | 349 | 28.512 | 43.656 | 71.842 | 1.00 | 26.02 | S |
| HETATM | 5778 | O | HOH | | 350 | −3.608 | 46.479 | 64.026 | 1.00 | 26.28 | S |
| HETATM | 5779 | O | HOH | | 351 | 18.647 | 19.162 | 79.481 | 1.00 | 26.17 | S |
| HETATM | 5780 | O | HOH | | 352 | 12.855 | 17.307 | 56.658 | 1.00 | 32.65 | S |
| HETATM | 5781 | O | HOH | | 353 | 16.965 | 47.043 | 72.185 | 1.00 | 26.50 | S |
| HETATM | 5782 | O | HOH | | 354 | 46.472 | 53.211 | 62.085 | 1.00 | 22.70 | S |
| HETATM | 5783 | O | HOH | | 355 | 6.130 | 66.386 | 81.864 | 1.00 | 29.83 | S |
| HETATM | 5784 | O | HOH | | 356 | 17.023 | 31.539 | 84.996 | 1.00 | 29.14 | S |
| HETATM | 5785 | O | HOH | | 357 | 52.271 | 18.734 | 88.733 | 1.00 | 21.31 | S |
| HETATM | 5786 | O | HOH | | 358 | 3.431 | 85.917 | 55.488 | 1.00 | 30.69 | S |
| HETATM | 5787 | O | HOH | | 359 | 47.513 | 57.453 | 62.042 | 1.00 | 31.46 | S |
| HETATM | 5788 | O | HOH | | 360 | 49.812 | 43.839 | 53.980 | 1.00 | 30.62 | S |
| HETATM | 5789 | O | HOH | | 361 | 27.823 | 57.738 | 36.617 | 1.00 | 24.95 | S |
| HETATM | 5790 | O | HOH | | 362 | 19.405 | 44.543 | 62.636 | 1.00 | 30.93 | S |
| HETATM | 5791 | O | HOH | | 363 | 41.234 | 43.276 | 72.913 | 1.00 | 26.38 | S |
| HETATM | 5792 | O | HOH | | 364 | 26.833 | 62.994 | 70.680 | 1.00 | 28.52 | S |
| HETATM | 5793 | O | HOH | | 365 | 27.182 | 44.067 | 51.006 | 1.00 | 27.75 | S |
| HETATM | 5794 | O | HOH | | 366 | 24.149 | 20.822 | 61.954 | 1.00 | 31.51 | S |
| HETATM | 5795 | O | HOH | | 367 | −9.756 | 71.124 | 69.912 | 1.00 | 32.62 | S |
| HETATM | 5796 | O | HOH | | 368 | 45.858 | 44.757 | 72.492 | 1.00 | 29.91 | S |
| HETATM | 5797 | O | HOH | | 369 | 33.879 | 40.394 | 64.911 | 1.00 | 32.97 | S |
| HETATM | 5798 | O | HOH | | 370 | 13.310 | 46.852 | 55.730 | 1.00 | 44.28 | S |
| HETATM | 5799 | O | HOH | | 371 | −9.512 | 81.831 | 59.288 | 1.00 | 35.66 | S |
| HETATM | 5800 | O | HOH | | 372 | 16.054 | 62.984 | 67.746 | 1.00 | 26.55 | S |
| HETATM | 5801 | O | HOH | | 373 | 2.579 | 25.777 | 86.480 | 1.00 | 25.93 | S |
| HETATM | 5802 | O | HOH | | 374 | 37.690 | 47.424 | 44.004 | 1.00 | 31.19 | S |
| HETATM | 5803 | O | HOH | | 375 | 16.450 | 50.504 | 70.478 | 1.00 | 34.17 | S |
| HETATM | 5804 | O | HOH | | 376 | 35.458 | 22.344 | 96.236 | 1.00 | 25.63 | S |
| HETATM | 5805 | O | HOH | | 377 | 34.462 | 52.915 | 41.693 | 1.00 | 27.64 | S |
| HETATM | 5806 | O | HOH | | 378 | 32.107 | 48.371 | 68.650 | 1.00 | 28.03 | S |
| HETATM | 5807 | O | HOH | | 379 | 2.262 | 22.313 | 58.634 | 1.00 | 26.71 | S |
| HETATM | 5808 | O | HOH | | 380 | 46.663 | 48.532 | 62.948 | 1.00 | 26.84 | S |
| HETATM | 5809 | O | HOH | | 381 | 31.217 | 71.451 | 67.457 | 1.00 | 26.53 | S |
| HETATM | 5810 | O | HOH | | 382 | 9.527 | 86.141 | 58.542 | 1.00 | 32.63 | S |
| HETATM | 5811 | O | HOH | | 383 | −1.251 | 58.714 | 45.772 | 1.00 | 30.00 | S |
| HETATM | 5812 | O | HOH | | 384 | −12.782 | 83.184 | 61.530 | 1.00 | 26.20 | S |
| HETATM | 5813 | O | HOH | | 385 | −3.103 | 25.877 | 69.084 | 1.00 | 37.65 | S |
| HETATM | 5814 | O | HOH | | 386 | 31.264 | 29.174 | 82.944 | 1.00 | 18.02 | S |
| HETATM | 5815 | O | HOH | | 387 | 51.298 | 37.662 | 64.650 | 1.00 | 29.24 | S |
| HETATM | 5816 | O | HOH | | 388 | 52.347 | 19.105 | 82.444 | 1.00 | 31.87 | S |
| HETATM | 5817 | O | HOH | | 389 | 47.795 | 60.859 | 62.970 | 1.00 | 28.59 | S |
| HETATM | 5818 | O | HOH | | 390 | −2.415 | 78.690 | 54.335 | 1.00 | 42.17 | S |
| HETATM | 5819 | O | HOH | | 391 | −4.825 | 38.135 | 73.016 | 1.00 | 28.28 | S |
| HETATM | 5820 | O | HOH | | 392 | 30.091 | 24.435 | 88.487 | 1.00 | 33.18 | S |
| HETATM | 5821 | O | HOH | | 393 | 44.207 | 58.354 | 69.531 | 1.00 | 32.91 | S |
| HETATM | 5822 | O | HOH | | 394 | 38.691 | 75.528 | 63.377 | 1.00 | 30.69 | S |
| HETATM | 5823 | O | HOH | | 395 | 39.913 | 52.414 | 74.017 | 1.00 | 31.71 | S |
| HETATM | 5824 | O | HOH | | 396 | 38.314 | 49.306 | 90.407 | 1.00 | 34.32 | S |
| HETATM | 5825 | O | HOH | | 397 | 20.502 | 66.613 | 39.713 | 1.00 | 42.19 | S |
| HETATM | 5826 | O | HOH | | 398 | 25.147 | 18.755 | 83.341 | 1.00 | 29.10 | S |
| HETATM | 5827 | O | HOH | | 399 | −0.076 | 51.795 | 70.452 | 1.00 | 28.90 | S |
| HETATM | 5828 | O | HOH | | 400 | 49.363 | 42.049 | 83.213 | 1.00 | 29.62 | S |
| HETATM | 5829 | O | HOH | | 401 | 15.592 | 64.559 | 70.535 | 1.00 | 26.12 | S |
| HETATM | 5830 | O | HOH | | 402 | 26.578 | 24.947 | 55.112 | 1.00 | 25.97 | S |
| HETATM | 5831 | O | HOH | | 403 | −8.084 | 55.954 | 57.095 | 1.00 | 33.29 | S |
| HETATM | 5832 | O | HOH | | 404 | 36.820 | 61.329 | 42.488 | 1.00 | 30.05 | S |
| HETATM | 5833 | O | HOH | | 405 | 7.415 | 48.585 | 77.749 | 1.00 | 24.99 | S |
| HETATM | 5834 | O | HOH | | 406 | 25.689 | 55.666 | 53.424 | 1.00 | 20.01 | S |
| HETATM | 5835 | O | HOH | | 407 | 32.946 | 24.931 | 94.324 | 1.00 | 30.42 | S |
| HETATM | 5836 | O | HOH | | 408 | 41.539 | 50.261 | 74.134 | 1.00 | 33.54 | S |
| HETATM | 5837 | O | HOH | | 409 | 51.748 | 35.764 | 82.888 | 1.00 | 35.79 | S |
| HETATM | 5838 | O | HOH | | 410 | 7.943 | 23.356 | 84.400 | 1.00 | 32.57 | S |
| HETATM | 5839 | O | HOH | | 411 | 23.385 | 66.975 | 34.509 | 1.00 | 36.51 | S |
| HETATM | 5840 | O | HOH | | 412 | 7.358 | 87.866 | 55.944 | 1.00 | 30.25 | S |
| HETATM | 5841 | O | HOH | | 413 | 32.230 | 44.633 | 89.785 | 1.00 | 26.34 | S |
| HETATM | 5842 | O | HOH | | 414 | 37.062 | 50.798 | 80.344 | 1.00 | 23.81 | S |
| HETATM | 5843 | O | HOH | | 415 | 13.722 | 31.495 | 83.244 | 1.00 | 36.71 | S |
| HETATM | 5844 | O | HOH | | 416 | 48.333 | 37.125 | 53.258 | 1.00 | 27.43 | S |
| HETATM | 5845 | O | HOH | | 417 | 5.105 | 57.495 | 77.924 | 1.00 | 31.33 | S |
| HETATM | 5846 | O | HOH | | 418 | 14.083 | 56.374 | 63.702 | 1.00 | 22.16 | S |
| HETATM | 5847 | O | HOH | | 419 | 20.352 | 39.273 | 88.123 | 1.00 | 24.90 | S |
| HETATM | 5848 | O | HOH | | 420 | −5.518 | 47.712 | 48.626 | 1.00 | 24.68 | S |
| HETATM | 5849 | O | HOH | | 421 | 54.558 | 25.326 | 91.260 | 1.00 | 26.78 | S |
| HETATM | 5850 | O | HOH | | 422 | 48.814 | 48.270 | 60.240 | 1.00 | 29.58 | S |
| HETATM | 5851 | O | HOH | | 423 | 33.313 | 11.383 | 80.286 | 1.00 | 28.99 | S |
| HETATM | 5852 | O | HOH | | 424 | 28.261 | 45.283 | 68.053 | 1.00 | 25.46 | S |
| HETATM | 5853 | O | HOH | | 425 | 22.290 | 42.258 | 72.671 | 1.00 | 28.26 | S |
| HETATM | 5854 | O | HOH | | 426 | 52.438 | 27.074 | 63.707 | 1.00 | 34.40 | S |
| HETATM | 5855 | O | HOH | | 427 | 31.754 | 47.487 | 66.246 | 1.00 | 27.45 | S |
| HETATM | 5856 | O | HOH | | 428 | 12.111 | 84.452 | 61.056 | 1.00 | 33.36 | S |
| HETATM | 5857 | O | HOH | | 429 | 16.950 | 44.945 | 81.922 | 1.00 | 31.47 | S |
| HETATM | 5858 | O | HOH | | 430 | 14.816 | 18.662 | 64.710 | 1.00 | 23.83 | S |
| HETATM | 5859 | O | HOH | | 431 | 7.262 | 48.466 | 54.597 | 1.00 | 22.97 | S |
| HETATM | 5860 | O | HOH | | 432 | 24.448 | 56.419 | 71.491 | 1.00 | 31.05 | S |
| HETATM | 5861 | O | HOH | | 433 | 50.864 | 27.938 | 95.490 | 1.00 | 27.44 | S |
| HETATM | 5862 | O | HOH | | 434 | 22.209 | 70.006 | 74.880 | 1.00 | 30.49 | S |
| HETATM | 5863 | O | HOH | | 435 | 30.382 | 51.808 | 82.387 | 1.00 | 33.92 | S |
| HETATM | 5864 | O | HOH | | 436 | 10.891 | 17.472 | 67.500 | 1.00 | 32.30 | S |
| HETATM | 5865 | O | HOH | | 437 | −4.206 | 45.387 | 66.335 | 1.00 | 22.29 | S |
| HETATM | 5866 | O | HOH | | 438 | 4.363 | 65.211 | 83.545 | 1.00 | 29.91 | S |
| HETATM | 5867 | O | HOH | | 439 | 6.551 | 29.885 | 82.623 | 1.00 | 28.44 | S |
| HETATM | 5868 | O | HOH | | 440 | 34.706 | 49.048 | 87.383 | 1.00 | 31.56 | S |
| HETATM | 5869 | O | HOH | | 441 | 36.740 | 32.287 | 80.773 | 1.00 | 29.36 | S |
| HETATM | 5870 | O | HOH | | 442 | 9.146 | 25.692 | 51.437 | 1.00 | 26.98 | S |
| HETATM | 5871 | O | HOH | | 443 | 41.058 | 20.869 | 67.860 | 1.00 | 33.47 | S |
| HETATM | 5872 | O | HOH | | 444 | 36.930 | 34.754 | 97.436 | 1.00 | 29.99 | S |
| HETATM | 5873 | O | HOH | | 445 | 10.466 | 65.553 | 80.919 | 1.00 | 27.51 | S |
| HETATM | 5874 | O | HOH | | 446 | 10.247 | 68.199 | 79.963 | 1.00 | 22.26 | S |
| HETATM | 5875 | O | HOH | | 447 | 44.182 | 69.249 | 56.022 | 1.00 | 37.49 | S |
| HETATM | 5876 | O | HOH | | 448 | 42.394 | 76.514 | 62.114 | 1.00 | 42.53 | S |
| HETATM | 5877 | O | HOH | | 449 | 33.807 | 35.678 | 61.492 | 1.00 | 34.37 | S |
| HETATM | 5878 | O | HOH | | 450 | 31.125 | 56.075 | 37.831 | 1.00 | 34.39 | S |
| HETATM | 5879 | O | HOH | | 451 | 28.837 | 65.136 | 36.172 | 1.00 | 37.53 | S |
| HETATM | 5880 | O | HOH | | 452 | 13.841 | 48.682 | 74.456 | 1.00 | 27.24 | S |
| HETATM | 5881 | O | HOH | | 453 | 29.376 | 42.624 | 57.105 | 1.00 | 34.13 | S |
| HETATM | 5882 | O | HOH | | 454 | 18.828 | 27.068 | 82.949 | 1.00 | 31.73 | S |
| HETATM | 5883 | O | HOH | | 455 | −1.955 | 24.801 | 51.248 | 1.00 | 42.82 | S |
| HETATM | 5884 | O | HOH | | 456 | 13.393 | 54.549 | 53.825 | 1.00 | 26.75 | S |
| HETATM | 5885 | O | HOH | | 457 | 17.875 | 33.363 | 87.041 | 1.00 | 26.45 | S |
| HETATM | 5886 | O | HOH | | 458 | 6.210 | 17.099 | 62.182 | 1.00 | 32.00 | S |
| HETATM | 5887 | O | HOH | | 459 | 18.444 | 64.386 | 72.960 | 1.00 | 28.23 | S |
| HETATM | 5888 | O | HOH | | 460 | 10.531 | 54.911 | 59.220 | 1.00 | 29.49 | S |
| HETATM | 5889 | O | HOH | | 461 | 32.104 | 27.003 | 90.350 | 1.00 | 29.63 | S |
| HETATM | 5890 | O | HOH | | 462 | 20.301 | 82.850 | 69.667 | 1.00 | 33.03 | S |
| HETATM | 5891 | O | HOH | | 463 | 22.126 | 55.570 | 65.304 | 1.00 | 45.31 | S |
| HETATM | 5892 | O | HOH | | 464 | 45.638 | 67.376 | 51.406 | 1.00 | 32.10 | S |
| HETATM | 5893 | O | HOH | | 465 | 44.388 | 22.559 | 93.130 | 1.00 | 31.86 | S |
| HETATM | 5894 | O | HOH | | 466 | 20.588 | 57.732 | 66.080 | 1.00 | 21.54 | S |
| HETATM | 5895 | O | HOH | | 467 | 21.553 | 19.952 | 54.986 | 1.00 | 34.25 | S |
| HETATM | 5896 | O | HOH | | 468 | 32.802 | 52.785 | 74.279 | 1.00 | 25.10 | S |
| HETATM | 5897 | O | HOH | | 469 | 0.465 | 27.401 | 79.288 | 1.00 | 34.07 | S |
| HETATM | 5898 | O | HOH | | 470 | 23.603 | 26.510 | 92.583 | 1.00 | 29.83 | S |
| HETATM | 5899 | O | HOH | | 471 | 45.149 | 18.861 | 87.060 | 1.00 | 32.43 | S |
| HETATM | 5900 | O | HOH | | 472 | 20.682 | 67.559 | 47.684 | 1.00 | 28.73 | S |
| HETATM | 5901 | O | HOH | | 473 | 5.995 | 80.881 | 49.929 | 1.00 | 30.52 | S |
| HETATM | 5902 | O | HOH | | 474 | 54.013 | 49.956 | 53.642 | 1.00 | 30.93 | S |
| HETATM | 5903 | O | HOH | | 475 | 33.544 | 24.991 | 89.225 | 1.00 | 31.89 | S |
| HETATM | 5904 | O | HOH | | 476 | −4.568 | 24.904 | 65.011 | 1.00 | 43.95 | S |
| HETATM | 5905 | O | HOH | | 477 | 28.654 | 54.661 | 71.967 | 1.00 | 30.00 | S |
| HETATM | 5906 | O | HOH | | 478 | 25.831 | 20.082 | 51.685 | 1.00 | 40.99 | S |
| HETATM | 5907 | O | HOH | | 479 | 10.774 | 58.617 | 46.886 | 1.00 | 42.47 | S |
| HETATM | 5908 | O | HOH | | 480 | 37.711 | 71.026 | 63.766 | 1.00 | 30.13 | S |
| HETATM | 5909 | O | HOH | | 481 | 51.574 | 41.856 | 81.375 | 1.00 | 36.90 | S |
| HETATM | 5910 | O | HOH | | 482 | 16.572 | 34.355 | 54.732 | 1.00 | 35.41 | S |
| HETATM | 5911 | O | HOH | | 483 | 16.115 | 52.107 | 48.600 | 1.00 | 29.72 | S |
| HETATM | 5912 | O | HOH | | 484 | 5.382 | 37.949 | 78.382 | 1.00 | 29.96 | S |
| HETATM | 5913 | O | HOH | | 485 | 0.044 | 42.656 | 77.540 | 1.00 | 34.93 | S |
| HETATM | 5914 | O | HOH | | 486 | −10.026 | 72.676 | 59.288 | 1.00 | 28.25 | S |
| HETATM | 5915 | O | HOH | | 487 | 11.624 | 74.577 | 64.069 | 1.00 | 25.05 | S |
| HETATM | 5916 | O | HOH | | 488 | 11.394 | 37.712 | 49.432 | 1.00 | 41.76 | S |
| HETATM | 5917 | O | HOH | | 489 | 47.699 | 39.505 | 98.150 | 1.00 | 39.78 | S |
| HETATM | 5918 | O | HOH | | 490 | 35.086 | 60.965 | 40.390 | 1.00 | 24.15 | S |
| HETATM | 5919 | O | HOH | | 491 | 35.224 | 34.850 | 57.610 | 1.00 | 30.18 | S |
| HETATM | 5920 | O | HOH | | 492 | 42.828 | 49.181 | 71.416 | 1.00 | 34.75 | S |
| HETATM | 5921 | O | HOH | | 493 | −13.200 | 81.783 | 65.795 | 1.00 | 38.11 | S |
| HETATM | 5922 | O | HOH | | 494 | 40.081 | 51.109 | 78.232 | 1.00 | 36.62 | S |
| HETATM | 5923 | O | HOH | | 495 | 5.846 | 14.625 | 70.234 | 1.00 | 36.79 | S |
| HETATM | 5924 | PG | GNP | | 1 | 28.411 | 35.425 | 84.765 | 1.00 | 4.49 | L |
| HETATM | 5925 | O1G | GNP | | 1 | 27.601 | 35.217 | 86.007 | 1.00 | 7.20 | L |
| HETATM | 5926 | O2G | GNP | | 1 | 27.530 | 36.012 | 83.720 | 1.00 | 7.99 | L |
| HETATM | 5927 | O3G | GNP | | 1 | 29.071 | 34.175 | 84.272 | 1.00 | 7.36 | L |
| HETATM | 5928 | N3B | GNP | | 1 | 29.594 | 36.560 | 85.157 | 1.00 | 6.12 | L |
| HETATM | 5929 | PB | GNP | | 1 | 30.781 | 37.049 | 84.105 | 1.00 | 4.30 | L |
| HETATM | 5930 | O1B | GNP | | 1 | 31.966 | 36.184 | 84.208 | 1.00 | 4.07 | L |
| HETATM | 5931 | O2B | GNP | | 1 | 30.263 | 37.152 | 82.704 | 1.00 | 7.33 | L |
| HETATM | 5932 | O3A | GNP | | 1 | 31.211 | 38.508 | 84.652 | 1.00 | 4.24 | L |
| HETATM | 5933 | PA | GNP | | 1 | 30.861 | 39.961 | 83.999 | 1.00 | 5.02 | L |
| HETATM | 5934 | O1A | GNP | | 1 | 31.540 | 40.153 | 82.668 | 1.00 | 5.29 | L |
| HETATM | 5935 | O2A | GNP | | 1 | 29.356 | 40.076 | 84.053 | 1.00 | 3.21 | L |
| HETATM | 5936 | O5* | GNP | | 1 | 31.585 | 40.908 | 85.039 | 1.00 | 2.69 | L |
| HETATM | 5937 | C5* | GNP | | 1 | 31.204 | 40.985 | 86.437 | 1.00 | 5.96 | L |
| HETATM | 5938 | C4* | GNP | | 1 | 31.552 | 42.354 | 87.000 | 1.00 | 5.21 | L |
| HETATM | 5939 | O4* | GNP | | 1 | 33.015 | 42.515 | 86.977 | 1.00 | 4.02 | L |
| HETATM | 5940 | C3* | GNP | | 1 | 31.009 | 43.564 | 86.214 | 1.00 | 5.66 | L |
| HETATM | 5941 | O3* | GNP | | 1 | 30.629 | 44.577 | 87.188 | 1.00 | 7.83 | L |
| HETATM | 5942 | C2* | GNP | | 1 | 32.177 | 44.024 | 85.393 | 1.00 | 5.97 | L |
| HETATM | 5943 | O2* | GNP | | 1 | 32.171 | 45.407 | 85.024 | 1.00 | 7.80 | L |
| HETATM | 5944 | C1* | GNP | | 1 | 33.388 | 43.698 | 86.273 | 1.00 | 5.69 | L |
| HETATM | 5945 | N9 | GNP | | 1 | 34.611 | 43.409 | 85.545 | 1.00 | 4.59 | L |
| HETATM | 5946 | C8 | GNP | | 1 | 34.825 | 42.548 | 84.494 | 1.00 | 4.90 | L |
| HETATM | 5947 | N7 | GNP | | 1 | 36.062 | 42.516 | 84.058 | 1.00 | 6.09 | L |
| HETATM | 5948 | C5 | GNP | | 1 | 36.697 | 43.424 | 84.890 | 1.00 | 4.53 | L |
| HETATM | 5949 | C6 | GNP | | 1 | 38.091 | 43.838 | 84.916 | 1.00 | 5.88 | L |
| HETATM | 5950 | O6 | GNP | | 1 | 39.035 | 43.493 | 84.219 | 1.00 | 5.86 | L |
| HETATM | 5951 | N1 | GNP | | 1 | 38.337 | 44.800 | 85.938 | 1.00 | 4.01 | L |
| HETATM | 5952 | C2 | GNP | | 1 | 37.385 | 45.294 | 86.807 | 1.00 | 6.39 | L |
| HETATM | 5953 | N2 | GNP | | 1 | 37.803 | 46.196 | 87.707 | 1.00 | 7.85 | L |
| HETATM | 5954 | N3 | GNP | | 1 | 36.098 | 44.903 | 86.772 | 1.00 | 7.00 | L |
| HETATM | 5955 | C4 | GNP | | 1 | 35.831 | 43.970 | 85.792 | 1.00 | 5.54 | L |
| HETATM | 5956 | PG | GNP | | 2 | 18.730 | 41.230 | 64.849 | 1.00 | 16.15 | L |
| HETATM | 5957 | O1G | GNP | | 2 | 19.656 | 42.133 | 64.075 | 1.00 | 17.56 | L |
| HETATM | 5958 | O2G | GNP | | 2 | 19.398 | 40.684 | 66.100 | 1.00 | 18.23 | L |
| HETATM | 5959 | O3G | GNP | | 2 | 18.220 | 40.122 | 63.978 | 1.00 | 14.63 | L |
| HETATM | 5960 | N3B | GNP | | 2 | 17.452 | 42.241 | 65.360 | 1.00 | 11.79 | L |
| HETATM | 5961 | PB | GNP | | 2 | 16.141 | 41.732 | 66.258 | 1.00 | 10.90 | L |
| HETATM | 5962 | O1B | GNP | | 2 | 15.132 | 41.027 | 65.431 | 1.00 | 9.24 | L |
| HETATM | 5963 | O2B | GNP | | 2 | 16.632 | 40.939 | 67.454 | 1.00 | 6.65 | L |
| HETATM | 5964 | O3A | GNP | | 2 | 15.444 | 43.112 | 66.727 | 1.00 | 8.67 | L |
| HETATM | 5965 | PA | GNP | | 2 | 15.517 | 43.869 | 68.169 | 1.00 | 8.45 | L |
| HETATM | 5966 | O1A | GNP | | 2 | 14.849 | 43.074 | 69.249 | 1.00 | 6.06 | L |
| HETATM | 5967 | O2A | GNP | | 2 | 16.954 | 44.244 | 68.360 | 1.00 | 8.92 | L |
| HETATM | 5968 | O5* | GNP | | 2 | 14.591 | 45.115 | 67.847 | 1.00 | 8.51 | L |
| HETATM | 5969 | C5* | GNP | | 2 | 14.931 | 46.107 | 66.835 | 1.00 | 9.10 | L |
| HETATM | 5970 | C4* | GNP | | 2 | 14.343 | 47.457 | 67.222 | 1.00 | 7.50 | L |
| HETATM | 5971 | O4* | GNP | | 2 | 12.872 | 47.343 | 67.228 | 1.00 | 10.32 | L |
| HETATM | 5972 | C3* | GNP | | 2 | 14.703 | 47.953 | 68.623 | 1.00 | 9.09 | L |
| HETATM | 5973 | O3* | GNP | | 2 | 14.836 | 49.398 | 68.518 | 1.00 | 9.10 | L |
| HETATM | 5974 | C2* | GNP | | 2 | 13.522 | 47.576 | 69.461 | 1.00 | 7.45 | L |
| HETATM | 5975 | O2* | GNP | | 2 | 13.319 | 48.328 | 70.656 | 1.00 | 10.08 | L |
| HETATM | 5976 | C1* | GNP | | 2 | 12.334 | 47.718 | 68.498 | 1.00 | 7.93 | L |
| HETATM | 5977 | N9 | GNP | | 2 | 11.230 | 46.820 | 68.783 | 1.00 | 8.04 | L |
| HETATM | 5978 | C8 | GNP | | 2 | 11.234 | 45.450 | 68.895 | 1.00 | 5.24 | L |
| HETATM | 5979 | N7 | GNP | | 2 | 10.064 | 44.917 | 69.161 | 1.00 | 4.30 | L |
| HETATM | 5980 | C5 | GNP | | 2 | 9.235 | 46.032 | 69.224 | 1.00 | 6.01 | L |
| HETATM | 5981 | C6 | GNP | | 2 | 7.816 | 46.102 | 69.483 | 1.00 | 6.08 | L |
| HETATM | 5982 | O6 | GNP | | 2 | 7.010 | 45.206 | 69.705 | 1.00 | 5.32 | L |
| HETATM | 5983 | N1 | GNP | | 2 | 7.339 | 47.455 | 69.464 | 1.00 | 7.07 | L |
| HETATM | 5984 | C2 | GNP | | 2 | 8.125 | 48.572 | 69.230 | 1.00 | 5.52 | L |
| HETATM | 5985 | N2 | GNP | | 2 | 7.505 | 49.769 | 69.254 | 1.00 | 9.02 | L |
| HETATM | 5986 | N3 | GNP | | 2 | 9.446 | 48.489 | 68.985 | 1.00 | 7.38 | L |
| HETATM | 5987 | C4 | GNP | | 2 | 9.928 | 47.190 | 68.999 | 1.00 | 7.31 | L |
| HETATM | 5988 | PG | GNP | | 3 | 24.135 | 53.026 | 59.854 | 1.00 | 15.72 | L |
| HETATM | 5989 | O1G | GNP | | 3 | 23.257 | 51.800 | 59.888 | 1.00 | 15.22 | L |
| HETATM | 5990 | O2G | GNP | | 3 | 23.439 | 54.227 | 60.463 | 1.00 | 17.32 | L |
| HETATM | 5991 | O3G | GNP | | 3 | 24.598 | 53.312 | 58.461 | 1.00 | 15.61 | L |
| HETATM | 5992 | N3B | GNP | | 3 | 25.467 | 52.652 | 60.843 | 1.00 | 13.00 | L |
| HETATM | 5993 | PB | GNP | | 3 | 26.744 | 53.681 | 61.136 | 1.00 | 11.17 | L |
| HETATM | 5994 | O1B | GNP | | 3 | 27.766 | 53.628 | 60.074 | 1.00 | 8.59 | L |
| HETATM | 5995 | O2B | GNP | | 3 | 26.205 | 55.065 | 61.423 | 1.00 | 8.90 | L |
| HETATM | 5996 | O3A | GNP | | 3 | 27.443 | 53.033 | 62.452 | 1.00 | 9.02 | L |
| HETATM | 5997 | PA | GNP | | 3 | 27.292 | 53.494 | 64.004 | 1.00 | 9.39 | L |
| HETATM | 5998 | O1A | GNP | | 3 | 27.898 | 54.851 | 64.237 | 1.00 | 9.43 | L |
| HETATM | 5999 | O2A | GNP | | 3 | 25.842 | 53.288 | 64.321 | 1.00 | 8.81 | L |
| HETATM | 6000 | O5* | GNP | | 3 | 28.237 | 52.415 | 64.676 | 1.00 | 9.77 | L |
| HETATM | 6001 | C5* | GNP | | 3 | 27.936 | 50.994 | 64.636 | 1.00 | 9.80 | L |
| HETATM | 6002 | C4* | GNP | | 3 | 28.484 | 50.327 | 65.883 | 1.00 | 10.71 | L |
| HETATM | 6003 | O4* | GNP | | 3 | 29.950 | 50.425 | 65.833 | 1.00 | 9.44 | L |
| HETATM | 6004 | C3* | GNP | | 3 | 28.083 | 50.975 | 67.217 | 1.00 | 11.06 | L |
| HETATM | 6005 | O3* | GNP | | 3 | 27.905 | 49.887 | 68.178 | 1.00 | 11.00 | L |
| HETATM | 6006 | C2* | GNP | | 3 | 29.259 | 51.831 | 67.575 | 1.00 | 9.85 | L |
| HETATM | 6007 | O2* | GNP | | 3 | 29.441 | 52.111 | 68.964 | 1.00 | 10.34 | L |
| HETATM | 6008 | C1* | GNP | | 3 | 30.462 | 51.065 | 66.999 | 1.00 | 9.99 | L |
| HETATM | 6009 | N9 | GNP | | 3 | 31.573 | 51.898 | 66.573 | 1.00 | 10.98 | L |
| HETATM | 6010 | C8 | GNP | | 3 | 31.585 | 52.959 | 65.689 | 1.00 | 11.83 | L |
| HETATM | 6011 | N7 | GNP | | 3 | 32.762 | 53.513 | 65.509 | 1.00 | 12.76 | L |
| HETATM | 6012 | C5 | GNP | | 3 | 33.583 | 52.754 | 66.340 | 1.00 | 10.19 | L |
| HETATM | 6013 | C6 | GNP | | 3 | 35.009 | 52.872 | 66.581 | 1.00 | 9.48 | L |
| HETATM | 6014 | O6 | GNP | | 3 | 35.836 | 53.664 | 66.114 | 1.00 | 7.40 | L |
| HETATM | 6015 | N1 | GNP | | 3 | 35.466 | 51.890 | 67.513 | 1.00 | 8.27 | L |
| HETATM | 6016 | C2 | GNP | | 3 | 34.663 | 50.935 | 68.123 | 1.00 | 7.93 | L |
| HETATM | 6017 | N2 | GNP | | 3 | 35.266 | 50.092 | 68.974 | 1.00 | 5.59 | L |
| HETATM | 6018 | N3 | GNP | | 3 | 33.347 | 50.835 | 67.891 | 1.00 | 9.28 | L |
| HETATM | 6019 | C4 | GNP | | 3 | 32.875 | 51.776 | 66.990 | 1.00 | 9.88 | L |
| HETATM | 6020 | PG | GNP | | 4 | 14.238 | 71.290 | 69.823 | 1.00 | 9.86 | L |
| HETATM | 6021 | O1G | GNP | | 4 | 15.050 | 72.284 | 70.595 | 1.00 | 11.23 | L |
| HETATM | 6022 | O2G | GNP | | 4 | 15.101 | 70.112 | 69.488 | 1.00 | 8.66 | L |
| HETATM | 6023 | O3G | GNP | | 4 | 13.632 | 71.881 | 68.586 | 1.00 | 9.75 | L |
| HETATM | 6024 | N3B | GNP | | 4 | 13.015 | 70.777 | 70.870 | 1.00 | 9.21 | L |
| HETATM | 6025 | PB | GNP | | 4 | 11.809 | 69.702 | 70.460 | 1.00 | 8.64 | L |
| HETATM | 6026 | O1B | GNP | | 4 | 10.613 | 70.397 | 69.942 | 1.00 | 7.78 | L |
| HETATM | 6027 | O2B | GNP | | 4 | 12.334 | 68.656 | 69.518 | 1.00 | 8.53 | L |
| HETATM | 6028 | O3A | GNP | | 4 | 11.378 | 69.035 | 71.875 | 1.00 | 7.21 | L |
| HETATM | 6029 | PA | GNP | | 4 | 11.713 | 67.544 | 72.448 | 1.00 | 9.34 | L |
| HETATM | 6030 | O1A | GNP | | 4 | 11.048 | 66.480 | 71.605 | 1.00 | 7.88 | L |
| HETATM | 6031 | O2A | GNP | | 4 | 13.212 | 67.483 | 72.594 | 1.00 | 7.22 | L |
| HETATM | 6032 | O5* | GNP | | 4 | 10.973 | 67.604 | 73.834 | 1.00 | 6.31 | L |
| HETATM | 6033 | C5* | GNP | | 4 | 11.372 | 68.537 | 74.879 | 1.00 | 7.39 | L |
| HETATM | 6034 | C4* | GNP | | 4 | 10.995 | 67.993 | 76.243 | 1.00 | 9.26 | L |
| HETATM | 6035 | O4* | GNP | | 4 | 9.529 | 67.879 | 76.318 | 1.00 | 9.13 | L |
| HETATM | 6036 | C3* | GNP | | 4 | 11.526 | 66.589 | 76.580 | 1.00 | 10.20 | L |
| HETATM | 6037 | O3* | GNP | | 4 | 11.890 | 66.609 | 77.983 | 1.00 | 10.07 | L |
| HETATM | 6038 | C2* | GNP | | 4 | 10.361 | 65.684 | 76.343 | 1.00 | 10.15 | L |
| HETATM | 6039 | O2* | GNP | | 4 | 10.351 | 64.487 | 77.112 | 1.00 | 11.27 | L |
| HETATM | 6040 | C1* | GNP | | 4 | 9.143 | 66.555 | 76.677 | 1.00 | 9.22 | L |
| HETATM | 6041 | N9 | GNP | | 4 | 7.943 | 66.216 | 75.934 | 1.00 | 7.55 | L |
| HETATM | 6042 | C8 | GNP | | 4 | 7.763 | 66.060 | 74.576 | 1.00 | 7.76 | L |
| HETATM | 6043 | N7 | GNP | | 4 | 6.539 | 65.746 | 74.219 | 1.00 | 8.91 | L |
| HETATM | 6044 | C5 | GNP | | 4 | 5.874 | 65.695 | 75.439 | 1.00 | 7.65 | L |
| HETATM | 6045 | C6 | GNP | | 4 | 4.481 | 65.396 | 75.718 | 1.00 | 8.26 | L |
| HETATM | 6046 | O6 | GNP | | 4 | 3.566 | 65.119 | 74.952 | 1.00 | 9.13 | L |
| HETATM | 6047 | N1 | GNP | | 4 | 4.194 | 65.451 | 77.118 | 1.00 | 7.65 | L |
| HETATM | 6048 | C2 | GNP | | 4 | 5.118 | 65.750 | 78.101 | 1.00 | 9.61 | L |
| HETATM | 6049 | N2 | GNP | | 4 | 4.671 | 65.757 | 79.370 | 1.00 | 10.24 | L |
| HETATM | 6050 | N3 | GNP | | 4 | 6.410 | 66.024 | 77.826 | 1.00 | 8.63 | L |
| HETATM | 6051 | C4 | GNP | | 4 | 6.711 | 65.978 | 76.482 | 1.00 | 7.36 | L |
| HETATM | 6052 | MG | MG | | 1 | 28.260 | 36.887 | 81.935 | 1.00 | 0.92 | M |
| HETATM | 6053 | MG | MG | | 2 | 18.788 | 40.289 | 67.968 | 1.00 | 36.51 | M |
| HETATM | 6054 | MG | MG | | 3 | 24.194 | 55.833 | 61.409 | 1.00 | 38.14 | M |
| HETATM | 6055 | MG | MG | | 4 | 14.295 | 68.234 | 68.773 | 1.00 | 0.92 | M |
| END | | | | | | | | | | | |

That which is claimed is:

1. A crystal of a C-terminally truncated human Rab9 having the three dimensional atomic coordinates of Table 4 or Table 6, wherein said Rab9 comprises amino acids 1-177 of SEQ ID NO:1.

2. The crystal of claim 1, having the three dimensional atomic coordinates of Table 4.

3. The crystal of claim 1, having the three dimensional atomic coordinates of Table 6.

4. A crystal of a Rab9-GDP complex, wherein the crystal effectively diffracts X-rays for the determination of the atomic coordinates of the Rab9-GDP complex to a resolution of less than about 1.75 Å.

5. The crystal of claim 4, wherein said resolution is between about 1.25 Å and about 1.75 Å.

6. The crystal of claim 4, wherein said resolution is about 1.25 Å.

7. The crystal of claim 4 having a space group of P₁ and a unit cell of dimensions a=38.40 Å, b=45.62 Å, c=51.22 Å, α=99.8°, β=107.2°, and γ=101.8°.

8. The crystal of claim 4, wherein said Rab9 has secondary structural elements that include six β-sheets and five α-helices, wherein said β-sheets are designated B1-B6 and wherein said α-helices are designated H1-H5, and wherein said β-sheets and said α-helices connect with a B1-H1-B2-B3-H2-B4-H3-B5-H4-B6-H5 topology.

9. A method of screening for an agent that modulates Rab9 activity comprising the steps of:
a) selecting a candidate agent by performing structure based drug design with the three-dimensional structure determined for a crystal of Rab9 according to claim 4, wherein said selecting is performed in conjunction with computer modeling;
b) contacting said candidate agent with Rab9; and
c) detecting the ability of said candidate agent to modulate Rab9.

10. The method according to claim 9, wherein said candidate agent is selected from a database.

11. The method according to claim 9, wherein said candidate agent is designed de novo.

12. The method according to claim 9, wherein said candidate agent is designed from a known modulator of Rab9 activity.

13. The method according to claim 9, wherein said candidate agent is an inhibitor of Rab9.

14. The method according to claim 13, wherein said candidate agent is a competitive inhibitor of Rab9.

15. The method according to claim 13, wherein said candidate agent is a non-competitive or uncompetitive inhibitor of Rab9.

16. The method according to claim 9, wherein said step of selecting a candidate agent comprises the steps of:
a) screening for chemical entities or fragments capable of associating with Rab9; and
b) assembling said chemical entities or fragments into a single molecule to provide the structure of said candidate agent.

17. A crystal of a Rab9-GppNHp complex, wherein the crystal effectively diffracts X-rays for the determination of the atomic coordinates of the Rab9-GppNHp complex to a resolution of less than about 2 Å.

18. The crystal of claim 17, wherein said resolution is between about 1.25 Å and about 2 Å.

19. The crystal of claim 17, wherein said resolution is about 1.73 Å.

20. The crystal of claim 17 having a space group of P2₁2₁2, and a unit cell of dimensions a=56.24 Å, b=76.60 Å, and c=174.35 Å.

21. The crystal of claim 17, wherein said Rab9 has secondary structural elements that include six β-sheets and five α-helices, wherein said β-sheets are designated B1-B6 and wherein said α-helices are designated H1-H5, and wherein said β-sheets and said α-helices connect with a B1-H1-B2-B3-H2-B4-H3-B5-H4-B6-H5 topology.

22. A method of screening for an agent that modulates Rab9 activity comprising the steps of:
a) selecting a candidate agent by performing structure based drug design with the three-dimensional structure determined for the crystal of a Rab9-GppNHp complex according to claim 17, wherein said selecting is performed in conjunction with computer modeling;
b) contacting said candidate agent with Rab9; and
c) detecting the ability of said candidate agent to modulate Rab9.

23. The method according to claim 22, wherein said candidate agent is selected from a database.

24. The method according to claim 22, wherein said candidate agent is designed de novo.

25. The method according to claim 22, wherein said candidate agent is designed from a known modulator of Rab9 activity.

26. The method according to claim 22, wherein said candidate agent is an inhibitor of Rab9.

27. The method according to claim 26, wherein said candidate agent is a competitive inhibitor of Rab9.

28. The method according to claim 26, wherein said candidate agent is a non-competitive or uncompetitive inhibitor of Rab9.

29. The method according to claim 22, wherein said step of selecting a candidate agent comprises the steps of:
a) screening for chemical entities or fragments capable of associating with Rab9; and
b) assembling said chemical entities or fragments into a single molecule to provide the structure of said candidate agent.

30. A method of screening for an agent that modulates Rab9 activity comprising the steps of:
a) providing a model of Rab9, said model including at least one of the binding sites defined by at least one of Table 2, FIG. 4, FIG. 5, FIG. 6B, or FIG. 8;
b) providing the structure of a candidate agent; and
c) fitting the candidate agent to said target site, including determining the interactions between the candidate agent and at least one of the binding sites defined by at least one of Table 2, FIG. 4, FIG. 5, FIG. 6B, or FIG. 8.

31. The method according to claim 30, comprising the further step of:
d) selecting the fitted candidate agent.

32. The method according to claim 31, comprising the further step of:
e) contacting the candidate agent with Rab9 to determine the ability of the candidate agent to interact with Rab9.

33. The method according to claim 31, comprising the further steps of:
e) forming a complex of Rab9 and said candidate agent; and
f) analyzing said complex to determine the ability of said candidate agent to interact with Rab9.

34. The method according to claim 33, wherein said step of analyzing comprises use of X-ray crystallography or NMR spectroscopy.

35. The method according to claim 30, wherein said binding sites are selected from the group consisting of Region I, Region II, Region III, Region IV, Region V, Region VI, and Region VII of a C-terminally truncated human Rab9, wherein said Rab9 comprises amino acids 1-177 of SEQ ID NO:1, and wherein Region I consists of amino acids 1 to 7 of SEQ ID NO:1, Region II consists of amino acids 33 to 43 of SEQ ID NO:1, Region III consists of amino acids 50 to 54 of SEQ ID NO:1, Region IV consists of amino acids 63 to 79 of SEQ ID NO:1, Region V consists of amino acids 108 to 117 of SEQ ID NO:1, Region VI consists of amino acids 128 to 130 of SEQ ID NO:1, and Region VII consists of amino acids 168 to 177 of SEQ ID NO:1.

36. The method according to claim 35, wherein said agent modulates Rab9 activity by inhibiting the binding of a Rab9-associated protein with Rab9.

37. The method according to claim 36, wherein said Rab9-associated protein is TIP47 or P40.

38. A method of screening for an agent that modulates Rab9 activity comprising the steps of:
a) providing a three-dimensional structure of Rab9 as defined by the atomic coordinate data of Table 4 or Table 6, wherein said Rab9 comprises amino acids 1-177 of SEQ ID NO:1;
b) employing said three-dimensional structure to design or select a candidate agent;
c) synthesizing said candidate agent; and
d) contacting said candidate agent with said Rab9 in the presence of a substrate to test the ability of said candidate agent to modulate the activity of said Rab9.

39. The method according to claim 38, wherein said candidate agent is selected from a database.

40. The method according to claim 38, wherein said candidate agent is designed de novo.

41. The method according to claim 38, wherein said candidate agent is designed from a known modulator of Rab9 activity.

42. The method according to claim 38, wherein said step of employing said three-dimensional structure to design or select said candidate agent comprises the steps of:
a) screening for chemical entities or fragments capable of associating with Rab9; and
b) assembling said identified chemical entities or fragments into a single molecule to provide the structure of said candidate agent.

43. The method according to claim 38, wherein said candidate agent is an inhibitor of Rab9.

44. The method according to claim 43, wherein said candidate agent is a competitive inhibitor of Rab9.

45. The method according to claim 43, wherein said candidate agent is a non-competitive or uncompetitive inhibitor of Rab9.

46. A method of screening for an agent that modulates Rab9 activity, comprising:
a) selecting or designing a candidate agent by performing structure based drug design with a computer system encoded with computer readable data comprising atomic coordinate data or binding site data or both, wherein said atomic coordinate data is defined by Table 4 or Table 6, and wherein said binding site data is defined by at least one of Table 2, FIG. 4, FIG. 5, FIG. 6B, or FIG. 8, wherein said selecting is performed in conjunction with computer modeling;
b) contacting the candidate agent with Rab9; and
c) detecting the ability of the candidate agent to modulate Rab9.

47. A computer system encoded with computer readable data comprising atomic coordinate data or binding site data or both, wherein said atomic coordinate data is defined by Table 4 or Table 6, and wherein said binding site data is defined by at least one of Table 2, FIG. 4, FIG. 5, FIG. 6B, or FIG. 8.

48. The computer system according to claim 47, wherein said binding sites are selected from the group consisting of Region I, Region II, Region III, Region IV, Region V, Region VI, and Region VII of the C-terminally truncated human Rab9 defined by the amino acid sequence of SEQ ID NO:1, wherein Region I consists of amino acids 1 to 7 of SEQ ID NO:1, Region II consists of amino acids 33 to 43 of SEQ ID NO:1, Region III consists of amino acids 50 to 54 of SEQ ID NO:1, Region IV consists of amino acids 63 to 79 of SEQ ID NO:1, Region V consists of amino acids 108 to 117 of SEQ ID NO:1, Region VI consists of amino acids 128 to 130 of SEQ ID NO:1, and Region VII consists of amino acids 168 to 177 of SEQ ID NO:1.

49. The computer system according to claim 48, wherein said binding sites mediate the binding of a Rab9-associated protein with Rab9.

50. The computer system according to claim 49, wherein said Rab9-associated protein is TIP47 or P40.

51. A computer-readable medium encoded with atomic coordinate data or binding site data or both, wherein said atomic coordinate data is defined by Table 4 or Table 6, and wherein said binding site data is defined by at least one of Table 2, FIG. 4, FIG. 5, FIG. 6B, or FIG. 8.

52. The computer-readable medium according to claim 51, wherein said binding sites are selected from the group consisting of Region I, Region II, Region III, Region IV, Region V, Region VI, and Region VII of the C-terminally truncated human Rab9 defined by the amino acid sequence of SEQ ID NO:1, wherein Region I consists of amino acids 1 to 7 of SEQ ID NO:1, Region II consists of amino acids 33 to 43 of SEQ ID NO:1, Region III consists of amino acids 50 to 54 of SEQ ID NO:1, Region IV consists of amino acids 63 to 79 of SEQ ID NO:1, Region V consists of amino acids 108 to 117 of SEQ ID NO:1, Region VI consists of amino acids 128 to 130 of SEQ ID NO:1, and Region VII consists of amino acids 168 to 177 of SEQ ID NO:1.

53. The computer-readable medium according to claim 52, wherein said binding sites mediate the binding of a Rab9-associated protein with Rab9.

54. The computer-readable medium according to claim 53, wherein said Rab9-associated protein is TIP47 or P40.

55. A method of using the computer-readable medium according to claim 51, wherein a graphical display software program is used to create an electronic file using said atomic coordinate data or said binding site data, wherein said electronic file can be visualized on a computer capable of representing said electronic file as a three dimensional image.


SEQUENCE LISTING (1 sequence)

SEQ ID NO: 1 - protein, 201 aa, Homo sapiens
MAGKSSLFKVILLGDGGVGKSSLMNRYVTNKFDTQLFHTIGVEFLNKDLEVDGHFVTMQIWDTAGQERFRSLRTPFYRGSDCCLLTFSVDDSQSFQNLSNWKKEFIYYADVKEPESFPFVILGNKIDISERQVSTEEAQAWCRDNGDYPYFETSAKDATNVAAAFEEAVRRVLATEDRSDHLIQTDTVNLHRKPKPSSSCC